(19) United States
(12) Patent Application Publication
(10) Pub. No.: US 2010/0150943 A1
(43) Pub. Date: Jun. 17, 2010

(54) IMMUNOGENIC COMPOSITIONS FOR GRAM POSITIVE BACTERIA

(75) Inventors: Guido Grandi, Siena (IT); John Telford, Siena (IT); Marirosa Mora, Siena (IT); Cesira Galeotti, Siena (IT); Daniela Rinaudo, Siena (IT); Andrea Guido Oreste Manetti, Siena (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: NOVARTIS AG, Basel (CH)

(21) Appl. No.: 12/375,042

(22) PCT Filed: Jul. 26, 2007
(86) PCT No.: PCT/IB07/04695

§ 371 Date: Feb. 3, 2009

Related U.S. Application Data
(60) Provisional application No. 60/833,238, filed on Jul. 26, 2006.

(51) Int. Cl. A61K 39/40; C07K 14/195; A61K 39/116; C12N 1/21; C12P 21/00; A61P 31/04

(52) U.S. Cl. 424/165.1; 530/350; 424/203.1; 435/252.3; 435/69.3; 424/164.1

(57) ABSTRACT

The invention relates to the identification of a new adhesin islands within the genomes of several Gram positive Streptococcus serotypes and isolates. Adhesin island polypeptides of the invention may be used in immunogenic compositions for prophylactic or therapeutic immunization against GAS, GBS, and S. pneumococcal infections.

### FIELD OF THE INVENTION

The invention relates to the identification of adhesin islands within the genome Streptococcus agalactiae (“GBS”) and the use of adhesin island amino acid sequences encoded by these adhesin islands in compositions for the treatment or prevention of GBS infection. Similar sequences have been identified in other Gram positive bacteria. The invention further includes immunogenic compositions comprising adhesin island amino acid sequences of Gram positive bacteria for the treatment or prevention of infection of Gram positive bacteria. Preferred immunogenic compositions of the invention include an adhesin island surface protein which may be formulated or purified in an oligomeric or pilus form.

### BACKGROUND OF THE INVENTION

GBS has emerged in the last 20 years as the major cause of neonatal sepsis and meningitis that affects 0.5-3 per 1000 live births, and an important cause of morbidity among older age groups affecting 5-8 per 100,000 of the population. Current disease management strategies rely on intrapartum antibiotics and neonatal monitoring which have reduced neonatal case mortality from >50% in the 1970's to less than 10% in the 1990's. Nevertheless, there is still considerable morbidity and mortality and the management is expensive. 15-35% of pregnant women are asymptomatic carriers and at high risk of transmitting the disease to their babies. Risk of neonatal infection is associated with low serotype specific maternal antibodies and high titers are believed to be protective. In addition, invasive GBS disease is increasingly recognized in elderly adults with underlying disease such as diabetes and cancer.

The “B” in “GBS” refers to the Lancefield classification, which is based on the antigenicity of a carbohydrate which is soluble in dilute acid and called the C carbohydrate. Lancefield identified 13 types of C carbohydrate, designated A to O, that could be serologically differentiated. The organisms that most commonly infect humans are found in groups A, B, D, and G. Within group B, strains can be divided into at least 9 serotypes (Ia, Ib, Ia/c, II, III, IV, V, VI, VII and VIII) based on the structure of their polysaccharide capsule. In the past, serotypes Ia, Ib, II, and III were equally prevalent in normal vaginal carriage and early onset sepsis in newborns. Type V GBS has emerged as an important cause of GBS infection in the USA, however, and strains of types VI and VIII have become prevalent among Japanese women.

The genome sequence of a serotype V strain 2603 V/R has been published (See Tettelin et al. (2002) Proc. Natl. Acad. Sci. USA, 2002 Sep. 17; 99(19):12391-6) and various polypeptides for use a vaccine antigens have been identified (WO 02/34771). The vaccines currently in clinical trials, however, are based primarily on polysaccharide antigens. These suffer from serotype-specificity and poor immunogenicity, and so there is a need for effective vaccines against S. agalactiae infection.

S. agalactiae is classified as a gram positive bacterium, a collection of about 21 genera of bacteria that colonize humans, have a generally spherical shape, a positive Gram stain reaction and lack endospores. Gram positive bacteria are frequent human pathogens and include Staphylococcus (such as S. aureus), Streptococcus (such as S. agalactiae (GBS), S. pyogenes (GAS), S. pneumoniae, S. mutans), Enterococcus (such as E. faecalis and E. faecium), Clostridium (such as C. difficile), Listeria (such as L. monocytogenes) and Corynebacterium (such as C. diphtheria).

It is an object of the invention to provide further and improved compositions for providing immunity against disease and/or infection of Gram positive bacteria. The compositions are based on the identification of adhesin islands within Streptococcal genomes and the use of amino acid sequences encoded by these islands in therapeutic or prophylactic compositions. The invention further includes compositions comprising immunogenic adhesin island proteins within other Gram positive bacteria in therapeutic or prophylactic compositions.

### SUMMARY OF THE INVENTION

Applicants have identified a new adhesin island, “GBS Adhesin Island 1,” “AI-1,” “GBS AI-1,” or “PI-1” within the genomes of several Group B Streptococcus serotypes and isolates. This adhesin island is thought to encode surface proteins which are important in the bacteria's virulence. In addition, Applicants have discovered that surface proteins within GBS Adhesin Islands form a previously unseen pilus structure on the surface of GBS bacteria. Amino acid sequences encoded by such GBS Adhesin Islands may be used in immunogenic compositions for the treatment or prevention of GBS infection.

A preferred immunogenic composition of the invention comprises an AI-1 surface protein, such as GBS 80, which may be formulated or purified in an oligomeric (pilus) form. In a preferred embodiment, the oligomeric form is a hyperoligomer. Electron micrographs depicting some of the first visualizations of this pilus structure in a wild type GBS strain are shown in FIGS. 16, 17, 49, and 50. In addition, Applicants have transformed a GBS strain with a plasmid comprising the AI surface protein

GBS 80 which resulted in increased production of that AI surface protein. The electron micrographs of this mutant GBS strain in FIGS. 13-15 reveal long, hyper-oligomeric structures comprising GBS 80 which appear to cover portions of the surface of the bacteria and stretch far out into the supernatant. These hyper-oligomeric pilus structures comprising a GBS AI surface protein may be purified or otherwise formulated for use in immunogenic compositions.

GBS AI-1 comprises a series of approximately five open reading frames encoding for a collection of amino acid sequences comprising surface proteins and sortases (“AI-1 proteins”). Specifically, AI-1 includes polynucleotide sequences encoding for two or more of GBS 80, GBS 104, GBS 52, SAG0647 and SAG0648. One or more of the AI-1 polynucleotide sequences may be replaced by a polynucleotide sequence coding for a fragment of the replaced ORF. Alternatively, one or more of the AI-1 open reading frames may be replaced by a sequence having sequence homology (sequence identity) to the replaced ORF.

AI-1 typically resides on an approximately 16.1 kb transposon-like element frequently inserted into the open reading frame for trmA. One or more of the AI-1 surface protein sequences typically include an LPXTG motif (e.g., SEQ ID NO:122) or other sortase substrate motif. The AI surface proteins of the invention may affect the ability of the GBS bacteria to adhere to and invade epithelial cells. AI surface proteins may also affect the ability of GBS to translocate through an epithelial cell layer.

Preferably, one or more AI surface proteins are capable of binding to or otherwise associating with an epithelial cell surface. AI surface proteins may also be able to bind to or associate with fibrinogen, fibronectin, or collagen.

The sortase proteins are thought to be involved in the secretion and anchoring of the LPXTG containing surface proteins. AI-1 may encode at least one surface protein. Alternatively, AI-1 may encode at least two surface proteins and at least one sortase. Preferably, AI-1 encodes for at least three surface proteins and at least two sortases. One or more of the surface proteins may include an LPXTG motif or other sortase substrate motif.

The GBS AI-1 protein of the composition may be selected from the group consisting of GBS 80, GBS 104, GBS 52, SAG0647 and SAG0648. GBS AI-1 surface proteins GBS 80 and GBS 104 are preferred for use in the immunogenic compositions of the invention.

In addition to the open reading frames encoding the AI-1 proteins, AI-1 may also include a divergently transcribed transcriptional regulator such as araC (i.e., the transcriptional regulator is located near or adjacent to the AI protein open reading frames, but it transcribed in the opposite direction). It is believed that araC may regulate the expression of the GBS AI operon. (See Korbel et al., Nature Biotechnology (2004) 22(7): 911-917 for a discussion of divergently transcribed regulators in E. coli).

A second adhesin island, “Adhesin Island-2,” “AI-2,” “GBS AI-2,” or “PI-2” has also been identified in numerous GBS serotypes. Amino acid sequences encoded by the open reading frames of AI-2 may also be used in immunogenic compositions for the treatment or prevention of GBS infection.

GBS AI-2 comprises a series of approximately five open reading frames encoding for a collection of amino acid sequences comprising surface proteins and sortases. Specifically, AI-2 includes open reading frames encoding for two or more of GBS 67, GBS 59, GBS 150, SAG1405, SAG1406, 01520, 01521, 01522, 01523, 01523, 01524 and 01525. The GBS AI-2 sequences may be divided into two subgroups. In one embodiment, AI-2 includes open reading frames encoding for two or more of GBS 67, GBS 59, GBS 150, SAG1405, and SAG1406. This collection of open reading frames may be generally referred to as GBS AI-2 subgroup 1 (or PI-2a). Alternatively, AI-2 may include open reading frames encoding for two or more of 01520, 01521, 01522, 01523, 01523, 01524 and 01525. This collection of open reading frames may be generally referred to as GBS AI-2 subgroup 2 (or PI-2b).

One or more of the AI-2 open reading frame polynucleotide sequences may be replaced by a polynucleotide sequence coding for a fragment of the replaced ORF. Alternatively, one or more of the AI-2 open reading frames may be replaced by a sequence having sequence homology (sequence identity) to the replaced ORF.

One or more of the AI-2 surface proteins typically include an LPXTG motif (e.g., SEQ ID NO:122) or other sortase substrate motif. These sortase proteins are thought to be involved in the secretion and anchoring of the LPXTG containing surface proteins. AI-2 may encode for at least one surface protein. Alternatively, AI-2 may encode for at least two surface proteins and at least one sortase. Preferably, AI-2 encodes for at least three surface proteins and at least two sortases. One or more of the surface proteins may include an LPXTG motif. The AI-2 protein of the composition may be selected from the group consisting of GBS 67, GBS 59, GBS 150, SAG1405, SAG1406, 01520, 01521, 01522, 01523, 01523, 01524 and 01525. AI-2 surface proteins GBS 67, GBS 59, and 01524 are preferred AI-2 proteins for use in the immunogenic compositions of the invention. GBS 67 or GBS 59 is particularly preferred.

GBS AI-2 may also include a divergently transcribed transcriptional regulator such as a RofA like protein (for example rogB). As in AI-1, rogB is thought to regulate the expression of the AI-2 operon.

The GBS AI proteins of the invention may be used in immunogenic compositions for prophylactic or therapeutic immunization against GBS infection. For example, the invention may include an immunogenic composition comprising one or more GBS AI-1 proteins and one or more GBS AI-2 proteins.

The immunogenic compositions may also be selected to provide protection against an increased range of GBS serotypes and strain isolates. For example, the immunogenic composition may comprise a first and second GBS AI protein, wherein a full length polynucleotide sequence encoding for the first GBS AI protein is not present in a genome comprising a full length polynucleotide sequence encoding for the second GBS AI protein. In addition, each antigen selected for use in the immunogenic compositions will preferably be present in the genomes of multiple GBS serotypes and strain isolates. Preferably, each antigen is present in the genomes of at least two (i.e., 3, 4, 5, 6, 7, 8, 9, 10, or more) GBS strain isolates. More preferably, each antigen is present in the genomes of at least two (i.e., at least 3, 4, 5 or more) GBS serotypes.

Within GBS AI-1, Applicants have found that Group B Streptococcus surface exposure of GBS 104 is dependent on the concurrent expression of GBS 80. It is thought that GBS 80 is involved in the transport or localization of GBS 104 to the surface of the bacteria. The two proteins may be oligomerized or otherwise chemically or physically associated. It is possible that this association involves a conformational change in GBS 104 that facilitates its transition to the surface of the GBS bacteria. In addition, one or more AI sortases may also be involved in this surface localization and chemical or physical association. Similar relationships are thought to exist within GBS AI-2. The compositions of the invention may therefore include at least two AI proteins, wherein the two AI proteins are physically or chemically associated. Preferably, the two AI proteins form an oligomer. Preferably, one or more of the AI proteins are in a hyper-oligomeric form. In one embodiment, the associated AI proteins may be purified or isolated from a GBS bacteria or recombinant host cell.

It is also an object of the invention to provide further and improved compositions for providing prophylactic or therapeutic protection against disease and/or infection of Gram positive bacteria. The compositions are based on the identification of adhesin islands within Streptococcal genomes and the use of amino acid sequences encoded by these islands in therapeutic or prophylactic compositions. The invention further includes compositions comprising immunogenic adhesin island proteins within other Gram positive bacteria in therapeutic or prophylactic compositions. Preferred Gram positive adhesin island proteins for use in the invention may be derived from Staphylococcus (such as S. aureus), Streptococcus (such as S. agalactiae (GBS), S. pyogenes (GAS), S. pneumoniae, S. mutans), Enterococcus (such as E. faecalis and E. faecium), Clostridium (such as C. difficile), Listeria (such as L. monocytogenes) and Corynebacterium (such as C. diphtheria). Preferably, the Gram positive adhesin island surface proteins are in oligomeric or hyperologimeric form.

For example, Applicants have identified adhesin islands within the genomes of several Group A Streptococcus serotypes and isolates. These adhesion islands are thought to encode surface proteins which are important in the bacteria's virulence, and Applicants have obtained the first electron micrographs revealing the presence of these adhesin island proteins in hyperoligomeric pilus structures on the surface of Group A Streptococcus.

Group A Streptococcus is a human specific pathogen which causes a wide variety of diseases ranging from pharyngitis and impetigo through life threatening invasive disease and necrotizing fasciitis. In addition, post-streptococcal autoimmune responses are still a major cause of cardiac pathology in children.

Group A Streptococcal infection of its human host can generally occur in three phases. The first phase involves attachment and/or invasion of the bacteria into host tissue and multiplication of the bacteria within the extracellular spaces. Generally this attachment phase begins in the throat or the skin. The deeper the tissue level infected, the more severe the damage that can be caused. In the second stage of infection, the bacteria secretes a soluble toxin that diffuses into the surrounding tissue or even systemically through the vasculature. This toxin binds to susceptible host cell receptors and triggers inappropriate immune responses by these host cells, resulting in pathology. Because the toxin can diffuse throughout the host, the necrosis directly caused by the GAS toxins may be physically located in sites distant from the bacterial infection. The final phase of GAS infection can occur long after the original bacteria have been cleared from the host system. At this stage, the host's previous immune response to the GAS bacteria due to cross reactivity between epitopes of a GAS surface protein, M, and host tissues, such as the heart. A general review of GAS infection can be found in Principles of Bacterial Pathogenesis, Groisman ed., Chapter 15 (2001).

In order to prevent the pathogenic effects associated with the later stages of GAS infection, an effective vaccine against GAS will preferably facilitate host elimination of the bacteria during the initial attachment and invasion stage.

Isolates of Group A Streptococcus are historically classified according to the M surface protein described above. The M protein is surface exposed trypsin-sensitive protein generally comprising two polypeptide chains complexed in an alpha helical formation. The carboxyl terminus is anchored in the cytoplasmic membrane and is highly conserved among all group A streptococci. The amino terminus, which extend through the cell wall to the cell surface, is responsible for the antigenic variability observed among the 80 or more serotypes of M proteins.

A second layer of classification is based on a variable, trypsin-resistant surface antigen, commonly referred to as the T-antigen. Decades of epidemiology based on M and T serological typing have been central to studies on the biological diversity and disease causing potential of Group A Streptococci. While the M-protein component and its inherent variability have been extensively characterized, even after five decades of study, there is still very little known about the structure and variability of T-antigens. Antisera to define T types is commercially available from several sources, including Sevapharma (sevapharma.cz/en).

The gene coding for one form of T-antigen, T-type 6, from an M6 strain of GAS (D741) has been cloned and characterized and maps to an approximately 11 kb highly variable pathogenicity island. Schneewind et al., J Bacteriol. (1990) 172(6):3310-3317. This island is known as the Fibronectin-binding, Collagen-binding T-antigen (FCT) region because it contains, in addition to the T6 coding gene (tee6), members of a family of genes coding for Extra Cellular Matrix (ECM) binding proteins. Bessen et al., Infection & Immunity (2002) 70(3):1159-1167. Several of the protein products of this gene family have been shown to directly bind either fibronectin and/or collagen. See Hanski et al., Infection & Immunity (1992) 60(12):5119-5125; Talay et al., Infection & Immunity (1992(60(9):3837-3844; Jaffe et al. (1996) 21(2):373-384; Rocha et al., Adv Exp Med Biol. (1997) 418:737-739; Kreikemeyer et al., J Biol Chem (2004) 279(16):15850-15859; Podbielski et al., Mol. Microbiol. (1999) 31(4):1051-64; and Kreikemeyer et al., Int. J. Med Microbiol (2004) 294(2-3):177-88. In some cases direct evidence for a role of these proteins in adhesion and invasion has been obtained.

Applicants raised antiserum against a recombinant product of the tee6 gene and used it to explore the expression of T6 in M6 strain 2724. In immunoblot of mutanolysin extracts of this strain, the antiserum recognized, in addition to a band corresponding to the predicted molecular mass of the product, very high molecular weight ladders ranging in mobility from about 100 kDa to beyond the resolution of the 3-8% gradient gels used.

This pattern of high molecular weight products is similar to that observed in immunoblots of the protein components of the pili identified in Streptococcus agalactiae (described above) and previously in Corynebacterium diphtheriae. Electron microscopy of strain M6_{—}2724 with antisera specific for the product of tee6 revealed abundant surface staining and long pilus like structures extending up to 700 nanometers from the bacterial surface, revealing that the T6 protein, one of the antigens recognized in the original Lancefield serotyping system, is located within a GAS Adhesin Island (GAS AI-1) and forms long covalently linked pilus structures.

Applicants have identified at least six different Group A Streptococcus Adhesin Islands. While these GAS AI sequences can be identified in numerous M types, Applicants have surprisingly discovered a correlation between the four main pilus subunits from the four different GAS AI types and specific T classifications. While other trypsin-resistant surface exposed proteins are likely also implicated in the T classification designations, the discovery of the role of the GAS adhesin islands (and the associated hyper-oligomeric pilus like structures) in T classification and GAS serotype variance has important implications for prevention and treatment of GAS infections. Applicants have identified protein components within each of the GAS adhesin islands which are associated with the pilus formation. These proteins are believed to be involved in the bacteria's initial adherence mechanisms Immunological recognition of these proteins may allow the host immune response to slow or prevent the bacteria's transition into the more pathogenic later stages of infection.

In addition, Applicants have discovered that the GBS pili structures appear to be implicated in the formation of biofilms (populations of bacteria growing on a surface, often enclosed in an exopolysaccharide matrix). Biofilms are generally associated with bacterial resistance, as antibiotic treatments and host immune response are frequently unable to eradicate all of the bacteria components of the biofilm. Direction of a host immune response against surface proteins exposed during the first steps of bacterial attachment (i.e., before complete biofilm formation) is preferable.

The invention therefore provides for improved immunogenic compositions against GAS infection which may target GAS bacteria during their initial attachment efforts to the host epithelial cells and may provide protection against a wide range of GAS serotypes. The immunogenic compositions of the invention include GAS AI surface proteins which may be formulated in an oligomeric, or hyperoligomeric (pilus) form. The immunogenic compositions of the invention may include one or more GAS AI surface proteins. The invention also includes combinations of GAS AI surface proteins. Combinations of GAS AI surface proteins may be selected from the same adhesin island or they may be selected from different GAS adhesin islands.

Amino acid sequence encoded by such GAS Adhesin Islands may be used in immunogenic compositions for the treatment or prevention of GAS infection. Preferred immunogenic compositions of the invention comprise a GAS AI surface protein which has been formulated or purified in an oligomeric (pilus) form. In a preferred embodiment, the oligomeric form is a hyperoligomer.

GAS Adhesin Islands generally include a series of open reading frames within a GAS genome that encode for a collection of surface proteins and sortases. A GAS Adhesin Island may encode for an amino acid sequence comprising at least one surface protein. The Adhesin Island, therefore, may encode at least one surface protein. Alternatively, a GAS Adhesin Island may encode for at least two surface proteins and at least one sortase. Preferably, a GAS Adhesin Island encodes for at least three surface proteins and at least two sortases. One or more of the surface proteins may include an LPXTG motif (e.g., SEQ ID NO:122) or other sortase substrate motif. One or more GAS AI surface proteins may participate in the formation of a pilus structure on the surface of the Gram positive bacteria.

GAS Adhesin Islands of the invention preferably include a divergently transcribed transcriptional regulator. The transcriptional regulator may regulate the expression of the GAS AI operon. Examples of transcriptional regulators found in GAS AI sequences include RofA and Nra.

The GAS AI surface proteins may bind or otherwise adhere to fibrinogen, fibronectin, or collagen. One or more of the GAS AI surface proteins may comprise a fimbrial structural subunit.

One or more of the GAS AI surface proteins may include an LPXTG motif or other sortase substrate motif. The LPXTG motif may be followed by a hydrophobic region and a charged C terminus, which are thought to retard the protein in the cell membrane to facilitate recognition by the membrane-localized sortase. See Barnett, et al., J. Bacteriology (2004) 186 (17): 5865-5875.

GAS AI sequences may be generally categorized as Type 1, Type 2, Type 3, or Type 4, depending on the number and type of sortase sequences within the island and the percentage identity of other proteins (with the exception of RofA and cpa) within the island. Schematics of the GAS adhesin islands are set forth in FIG. 51A and FIG. 162. “GAS Adhesin Island-1 or “GAS AI-1” comprises a series of approximately five open reading frames encoding for a collection of amino acid sequences comprising surface proteins and sortases (“GAS AI-1 proteins”). GAS AI-1 preferably comprises surface proteins, a srtB sortase and a rofA divergently transcribed transcriptional regulator. GAS AI-1 surface proteins may include a fibronectin binding protein, a collagen adhesion protein and a fimbrial structural subunit. The fimbrial structural subunit (also known as tee6) is thought to form the shaft portion of the pilus like structure, while the collagen adhesion protein (Cpa) is thought to act as an accessory protein facilitating the formation of the pilus structure, exposed on the surface of the bacterial capsule.

Specifically, GAS AI-1 includes polynucleotide sequences encoding for two or more of M6_Spy0157, M6_Spy0158, M6_Spy0159, M6_Spy0160, M6_Spy0161. The GAS AI-1 may also include polynucleotide sequences encoding for any one of CDC SS 410_fimbrial, ISS3650_fimbrial, DSM2071_fimbrial.

A preferred immunogenic composition of the invention comprises a GAS AI-1 surface protein which may be formulated or purified in an oligomeric (pilus) form. In a preferred embodiment, the oligomeric form is a hyperoligomer. The immunogenic composition of the invention may alternatively comprise an isolated GAS AI-1 surface protein in oligomeric (pilus) form. The oligomer or hyperoligomeric pilus structures comprising GAS AI-1 surface proteins may be purified or otherwise formulated for use in immunogenic compositions.

One or more of the GAS AI-1 polynucleotide sequences may be replaced by a polynucleotide sequence coding for a fragment of the replaced ORF. Alternatively, one or more of the GAS AI-1 open reading frames may be replaced by a sequence having sequence homology (sequence identity) to the replaced ORF.

One or more of the GAS AI-1 surface proteins typically include an LPXTG motif (e.g., SEQ ID NO:122) or other sortase substrate motif. These sortase proteins are thought to be involved in the secretion and anchoring of the LPXTG containing surface proteins. GAS AI-1 may encode for at least one surface protein. Alternatively, GAS AI-1 may encode for at least two surface proteins and at least one sortase. Preferably, GAS AI-1 encodes for at least three surface proteins and at least two sortases. One or more of the surface proteins may include an LPXTG motif.

GAS AI-1 preferably includes a srtB sortase. GAS srtB sortases may preferably anchor surface proteins with an LPSTG motif (SEQ ID NO:166), particularly where the motif is followed by a serine.

The GAS AI-1 protein of the composition may be selected from the group consisting of M6_Spy0157, M6_Spy0158, M6_Spy0159, M6_Spy0160 M6_Spy0161, CDC SS 410_fimbrial, ISS3650_fimbrial, and DSM2071fimbrial. GAS AI-1 surface proteins M6_Spy0157 (a fibronectin binding protein), M6_Spy0159 (a collagen adhesion protein, Cpa), M6_Spy0160 (a fimbrial structural subunit, tee6), CDC SS 410_fimbrial (a fimbrial structural subunit), ISS3650_fimbrial (a fimbrial structural subunit), and DSM2071_fimbrial (a fimbrial structural subunit) are preferred GAS AI-1 proteins for use in the immunogenic compositions of the invention. The fimbrial structural subunit tee6 and the collagen adhesion protein Cpa are preferred GAS AI-1 surface proteins. Preferably, each of these GAS AI-1 surface proteins includes an LPXTG sortase substrate motif, such as LPXTG (SEQ ID NO:122) or LPXSG (SEQ ID NO:134) (conservative replacement of threonine with serine).

In addition to the open reading frames encoding the GAS AI-1 proteins, GAS AI-1 may also include a divergently transcribed transcriptional regulator such as rofA (i.e., the transcriptional regulator is located near or adjacent to the GAS AI protein open reading frames, but it transcribed in the opposite direction).

The GAS AI-1 surface proteins may be used alone, in combination with other GAS AI-1 surface proteins or in combination with other GAS AI surface proteins. Preferably, the immunogenic compositions of the invention include the GAS AI-1 fimbrial structural subunit (tee6) and the GAS AI-1 collagen binding protein. Still more preferably, the immunogenic compositions of the invention include the GAS AI-1 fimbrial structural subunit (tee6).

A second GAS adhesion island, “GAS Adhesin Island-2” or “GAS AI-2,” has also been identified in GAS serotypes. Amino acid sequences encoded by the open reading frames of GAS AI-2 may also be used in immunogenic compositions for the treatment or prevention of GAS infection.

A preferred immunogenic composition of the invention comprises a GAS AI-2 surface protein which may be formulated or purified in an oligomeric (pilus) form. In a preferred embodiment, the oligomeric form is a hyperoligomer. A preferred immunogenic composition of the invention alternatively comprises an isolated GAS AI-2 surface protein in oligomeric (pilus) form. The oligomer or hyperoligomeric pilus structures comprising GAS AI-2 surface proteins may be purified or otherwise formulated for use in immunogenic compositions.

GAS AI-2 comprises a series of approximately eight open reading frames encoding for a collection of amino acid sequences comprising surface proteins and sortases (“GAS AI-2 proteins”). GAS AI-2 preferably comprises surface proteins, a srtB sortase, a srtC1 sortase and a rofA divergently transcribed transcriptional regulator.

Specifically, GAS AI-2 includes polynucleotide sequences encoding for two or more of GAS15, Spy0127, GAS16, GAS17, GAS18, Spy0131, Spy0133, and GAS20.

One or more of the GAS AI-2 polynucleotide sequences may be replaced by a polynucleotide sequence coding for a fragment of the replaced ORF. Alternatively, one or more of the GAS AI-2 open reading frames may be replaced by a sequence having sequence homology (sequence identity) to the replaced ORF.

One or more of the GAS AI-2 surface proteins typically include an LPXTG motif (e.g., SEQ ID NO:122) or other sortase substrate motif. These sortase proteins are thought to be involved in the secretion and anchoring of the LPXTG containing surface proteins. GAS AI-2 may encode for at least one surface protein. Alternatively, GAS AI-2 may encode for at least two surface proteins and at least one sortase. Preferably, GAS AI-2 encodes for at least three surface proteins and at least two sortases. One or more of the surface proteins may include an LPXTG motif.

GAS AI-2 preferably includes a srtB sortase and a srtC1 sortase. As discussed above, GAS srtB sortases may preferably anchor surface proteins with an LPSTG motif (SEQ ID NO:166), particularly where the motif is followed by a serine. GAS srtC1 sortase may preferentially anchor surface proteins with a V(P/V)PTG (SEQ ID NO:167) motif. GAS srtC1 may be differentially regulated by rofA.

The GAS AI-2 protein of the composition may be selected from the group consisting of GAS15, Spy0127, GAS16, GAS17, GAS18, Spy0131, Spy0133, and GAS20. GAS AI-2 surface proteins GAS15 (Cpa), GAS16 (thought to be a fimbrial protein, M1_{—}128), GAS18 (M1_Spy0130), and GAS20 are preferred for use in the immunogenic compositions of the invention. GAS 16 is thought to form the shaft portion of the pilus like structure, while GAS 15 (the collagen adhesion protein Cpa) and GAS 18 are thought to act as accessory proteins facilitating the formation of the pilus structure, exposed on the surface of the bacterial capsule. Preferably, each of these GAS AI-2 surface proteins includes an LPXTG sortase substrate motif, such as LPXTG (SEQ ID NO:122), VVXTG (SEQ ID NO:135), or EVXTG (SEQ ID NO:136).

In addition to the open reading frames encoding the GAS AI-2 proteins, GAS AI-2 may also include a divergently transcribed transcriptional regulator such as rofA (i.e., the transcriptional regulator is located near or adjacent to the GAS AI protein open reading frames, but it transcribed in the opposite direction).The GAS AI-2 surface proteins may be used alone, in combination with other GAS AI-2 surface proteins or in combination with other GAS AI surface proteins. Preferably, the immunogenic compositions of the invention include the GAS AI-2 fimbrial protein (GAS 16), the GAS AI-2 collagen binding protein (GAS 15) and GAS 18 (M1_Spy0130). More preferably, the immunogenic compositions of the invention include the GAS AI-2 fimbrial protein (GAS 16).

A third GAS adhesion island, “GAS Adhesin Island-3” or “GAS AI-3,” has also been identified in numerous GAS serotypes. Amino acid sequences encoded by the open reading frames of GAS AI-3 may also be used in immunogenic compositions for the treatment or prevention of GAS infection.

A preferred immunogenic composition of the invention comprises a GAS AI-3 surface protein which may be formulated or purified in an oligomeric (pilus) form. In a preferred embodiment, the oligomeric form is a hyperoligomer. A preferred immunogenic composition of the invention alternatively comprises an isolated GAS AI-3 surface protein in oligomeric (pilus) form. The oligomer or hyperoligomeric pilus structures comprising GAS AI-3 surface proteins may be purified or otherwise formulated for use in immunogenic compositions. GAS AI-3 comprises a series of approximately seven open reading frames encoding for a collection of amino acid sequences comprising surface proteins and sortases (“GAS AI-3 proteins”). GAS AI-3 preferably comprises surface proteins, a srtC2 sortase, and a Negative transcriptional regulator (Nra) divergently transcribed transcriptional regulator. GAS AI-3 surface proteins may include a collagen binding protein, a fimbrial protein, and a F2 like fibronectin-binding protein. GAS AI-3 surface proteins may also include a hypothetical surface protein. The fimbrial protein is thought to form the shaft portion of the pilus like structure, while the collagen adhesion protein (Cpa) and the hypothetical surface protein are thought to act as accessory proteins facilitating the formation of the pilus structure, exposed on the surface of the bacterial capsule. Preferred AI-3 surface proteins include the fimbrial protein, the collagen binding protein and the hypothetical protein. Preferably, each of these GAS AI-3 surface proteins include an LPXTG sortase substrate motif, such as LPXTG (SEQ ID NO:122), VPXTG (SEQ ID NO:137), QVXTG (SEQ ID NO:138) or LPXAG (SEQ ID NO:139).

Specifically, GAS AI-3 includes polynucleotide sequences encoding for two or more of SpyM3_{—}0098, SpyM3_{—}0099, SpyM3_{—}0100, SpyM3_{—}0101, SpyM3_{—}0102, SpyM3_{—}0103, SpyM3_{—}0104, Sps0100, Sps0101, Sps0102, Sps0103, Sps0104, Sps0105, Sps0106, orf78, orf79, orf80, orf81, orf82, orf83, orf84, spyM18_{—}0126, spyM18_{—}0127, spyM18_{—}0128, spyM18_{—}0129, spyM18_{—}0130, spyM18_{—}0131, spyM18_{—}0132, SpyoM01000156, SpyoM01000155, SpyoM01000154, SpyoM01000153, SpyoM01000152, SpyoM01000151, SpyoM01000150, SpyoM01000149, ISS3040_fimbrial, ISS3776_fimbrial, and ISS4959_fimbrial. In one embodiment, GAS AI-3 may include open reading frames encoding for two or more of SpyM3_{—}0098, SpyM3_{—}0099, SpyM3_{—}0100, SpyM3_{—}0101, SpyM3_{—}0102, SpyM3_{—}0103, and SpyM3_{—}0104. Alternatively, GAS AI-3 may include open reading frames encoding for two or more of Sps0100, Sps0101, Sps0102, Sps0103, Sps0104, Sps0105, and Sps0106. Alternatively, GAS AI-3 may include open reading frames encoding for two or more of orf78, orf79, orf80, orf81, orf82, orf83, and orf84. Alternatively, GAS AI-3 may include open reading frames encoding for two or more of spyM18_{—}0126, spyM18_{—}0127, spyM18_{—}0128, spyM18_{—}0129, spyM18_{—}0130, spyM18_{—}0131, and spyM18_{—}0132. Alternatively, GAS AI-3 may include open reading frames encoding for two or more of SpyoM01000156, SpyoM01000155, SpyoM01000154, SpyoM01000153, SpyoM01000152, SpyoM01000151, SpyoM01000150, and SpyoM01000149. Alternatively, GAS AI-1 may also include polynucleotide sequences encoding for any one of ISS3040_fimbrial, ISS3776_fimbrial, and ISS4959_fimbrial.

One or more of the GAS AI-3 polynucleotide sequences may be replaced by a polynucleotide sequence coding for a fragment of the replaced ORF. Alternatively, one or more of the GAS AI-3 open reading frames may be replaced by a sequence having sequence homology (sequence identity) to the replaced ORF.

One or more of the GAS AI-3 surface proteins typically include an LPXTG motif (e.g., SEQ ID NO:122) or other sortase substrate motif. These sortase proteins are thought to be involved in the secretion and anchoring of the LPXTG containing surface proteins. GAS AI-3 may encode for at least one surface protein. Alternatively, GAS AI-3 may encode for at least two surface proteins and at least one sortase. Preferably, GAS AI-3 encodes for at least three surface proteins and at least two sortases. One or more of the surface proteins may include an LPXTG motif.

GAS AI-3 preferably includes a srtC2 type sortase. GAS srtC2 type sortases may preferably anchor surface proteins with a QVPTG (SEQ ID NO:140) motif, particularly when the motif is followed by a hydrophobic region and a charged C terminus tail. GAS SrtC2 may be differentially regulated by Nra.

The GAS AI-3 protein of the composition may be selected from the group consisting of SpyM3_{—}0098, SpyM3_{—}0099, SpyM3_{—}0100, SpyM3_{—}0101, SpyM3_{—}0102, SpyM3_{—}0103, SpyM3_{—}0104, Sps0100, Sps0101, Sps0102, Sps0103, Sps0104, Sps0105, Sps0106, orf78, orf79, orf80, orf81, orf82, orf83, orf84, spyM18_{—}0126, spyM18_{—}0127, spyM18_{—}0128, spyM18_{—}0129, spyM18_{—}0130, spyM18_{—}0131, spyM18_{—}0132, SpyoM01000156, SpyoM01000155, SpyoM01000154, SpyoM01000153, SpyoM01000152, SpyoM01000151, SpyoM01000150, SpyoM01000149, ISS3040_fimbrial, ISS3776_fimbrial, and ISS4959_fimbrial. GAS AI-3 surface proteins SpyM3_{—}0098, SpyM3_{—}0100, SpyM3_{—}0102, SpyM3_{—}0104, SPs0100, SPs0102, SPs0104, SPs0106, orf78, orf80, orf82, orf84, spyM18_{—}0126, spyM18_{—}0128, spyM18_{—}0130, spyM18_{—}0132, SpyoM01000155, SpyoM01000153, SpyoM01000151, SpyoM01000149, ISS3040_fimbrial, ISS3776_fimbrial, and ISS4959_fimbrial are preferred GAS AI-3 proteins for use in the immunogenic compositions of the invention.

In addition to the open reading frames encoding the GAS AI-3 proteins, GAS AI-3 may also include a transcriptional regulator such as Nra.

GAS AI-3 may also include a LepA putative signal peptidase I protein.

The GAS AI-3 surface proteins may be used alone, in combination with other GAS AI-3 surface proteins or in combination with other GAS AI surface proteins. Preferably, the immunogenic compositions of the invention include the GAS AI-3 fimbrial protein, the GAS AI-3 collagen binding protein, the GAS AI-3 surface protein (such as SpyM3_{—}0102, M3_Sps0104, M5_orf82, or spyM18_{—}0130), and fibronectin binding protein PrtF2. More preferably, the immunogenic compositions of the invention include the GAS AI-3 fimbrial protein, the GAS AI-3 collagen binding protein, and the GAS AI-3 surface protein. Still more preferably, the immunogenic compositions of the invention include the GAS AI-3 fimbrial protein.

Representative examples of the GAS AI-3 fimbrial protein include SpyM3_{—}0100, M3_Sps0102, M5_orf80, spyM18_{—}128, SpyoM01000153, ISS3040_fimbrial, ISS3776_fimbrial, ISS4959_fimbrial.

Representative examples of the GAS AI-3 collagen binding protein include SpyM30098, M3_Sps0100, M5_orf 78, spyM18_{—}0126, and SpyoM01000155.

Representative examples of the GAS AI-3 fibronectin binding protein PrtF2 include SpyM30104, M3_Sps0106, M5_orf84 and spyM180132, and SpyoM01000149.

A fourth GAS adhesion island, “GAS Adhesin Island-4” or “GAS AI-4,” has also been identified in GAS serotypes. Amino acid sequences encoded by the open reading frames of GAS AI-4 may also be used in immunogenic compositions for the treatment or prevention of GAS infection.

A preferred immunogenic composition of the invention comprises a GAS AI-4 surface protein which may be formulated or purified in an oligomeric (pilus) form. In a preferred embodiment, the oligomeric form is a hyperoligomer. A preferred immunogenic composition of the invention alternatively comprises an isolated GAS AI-4 surface protein in oligomeric (pilus) form. The oligomer or hyperoligomeric pilus structures comprising GAS AI-3 surface proteins may be purified or otherwise formulated for use in immunogenic compositions. The oligomeric or hyperoligomeric pilus structures comprising GAS AI-4 surface proteins may be purified or otherwise formulated for use in immunogenic compositions.

GAS AI-4 comprises a series of approximately eight open reading frames encoding for a collection of amino acid sequences comprising surface proteins and sortases (“GAS AI-4 proteins”). This GAS adhesin island 4 (“GAS AI-4”) comprises surface proteins, a srtC2 sortase, and a RofA regulatory protein. GAS AI-4 surface proteins within may include a fimbrial protein, F1 and F2 like fibronectin-binding proteins, and a capsular polysaccharide adhesion protein (cpa). GAS AI-4 surface proteins may also include a hypothetical surface protein in an open reading frame (orf).

The fimbrial protein (EftLSL) is thought to form the shaft portion of the pilus like structure, while the collagen adhesion protein (Cpa) and the hypothetical protein are thought to act as accessory proteins facilitating the formation of the pilus structure, exposed on the surface of the bacterial capsule. Preferably, each of these GAS AI-4 surface proteins include an LPXTG sortase substrate motif, such as LPXTG (SEQ ID NO:122), VPXTG (SEQ ID NO:137), QVXTG (SEQ ID NO:138) or LPXAG (SEQ ID NO:139).

Specifically, GAS AI-4 includes polynucleotide sequences encoding for two or more of 19224134, 19224135, 19224136, 19224137, 19224138, 19224139, 19224140, and 19224141. A GAS AI-4 polynucleotide may also include polynucleotide sequences encoding for any one of 20010296_fimbrial, 20020069 fimbrial, CDC SS 635 fimbrial, ISS4883fimbrial, ISS4538fimbrial. One or more of the GAS AI-4 polynucleotide sequences may be replaced by a polynucleotide sequence coding for a fragment of the replaced ORF. Alternatively, one or more of the GAS AI-4 open reading frames may be replaced by a sequence having sequence homology (sequence identity) to the replaced ORF.

One or more of the GAS AI-4 surface proteins typically include an LPXTG motif (e.g., SEQ ID NO:122) or other sortase substrate motif. These sortase proteins are thought to be involved in the secretion and anchoring of the LPXTG containing surface proteins. GAS AI-4 may encode for at least one surface protein. Alternatively, GAS AI-4 may encode for at least two surface proteins and at least one sortase. Preferably, GAS AI-4 encodes for at least three surface proteins and at least two sortases. One or more of the surface proteins may include an LPXTG motif.

GAS AI-4 includes a SrtC2 type sortase. GAS SrtC2 type sortases may preferably anchor surface proteins with a QVPTG (SEQ ID NO:140) motif, particularly when the motif is followed by a hydrophobic region and a charged C terminus tail.

The GAS AI-4 protein of the composition may be selected from the group consisting of 19224134, 19224135, 19224136, 19224137, 19224138, 19224139, 19224140, 19224141, 20010296_fimbrial, 20020069_fimbrial, CDC SS 635_fimbrial, ISS4883_fimbrial, and ISS4538_fimbrial. GAS AI-4 surface proteins 19224134, 19224135, 19224137, 19224139, 19224141, 20010296_fimbrial, 20020069_fimbrial, CDC SS 635_fimbrial, ISS4883_fimbrial, ISS4538_fimbrial are preferred proteins for use in the immunogenic compositions of the invention.

In addition to the open reading frames encoding the GAS AI-4 proteins, GAS AI-4 may also include a divergently transcribed transcriptional regulator such as RofA (i.e., the transcriptional regulator is located near or adjacent to the AI protein open reading frames, but it transcribed in the opposite direction.

GAS AI-4 may also include a LepA putative signal peptidase I protein and a MsmRL protein. The GAS AI-4 surface proteins may be used alone, in combination with other GAS AI-4 surface proteins or in combination with other GAS AI surface proteins. Preferably, the immunogenic compositions of the invention include the GAS AI-4 fimbrial protein (EftLSL or 20010296_fimbrial, 20020069_fimbrial, CDC SS 635_fimbrial, ISS4883_fimbrial, or ISS4538_fimbrial), the GAS AI-4 collagen binding protein, the GAS AI-4 surface protein (such as M12 isolate A735 orf 2), and fibronectin binding protein PrtF1 and PrtF2. More preferably, the immunogenic compositions of the invention include the GAS AI-4 fimbrial protein, the GAS AI-4 collagen binding protein, and the GAS AI-4 surface protein. Still more preferably, the immunogenic compositions of the invention include the GAS AI-4 fimbrial protein.

A fifth GAS adhesion island, “GAS Adhesin Island-5” or “GAS AI-5,” has also been identified in GAS serotypes. Amino acid sequences encoded by the open reading frames of GAS AI-5 may also be used in immunogenic compositions for the treatment or prevention of GAS infection.

A preferred immunogenic composition of the invention comprises a GAS AI-5 surface protein which may be formulated or purified in an oligomeric (pilus) form. In a preferred embodiment, the oligomeric form is a hyperoligomer. A preferred immunogenic composition of the invention alternatively comprises an isolated GAS AI-5 surface protein in oligomeric (pilus) form. The oligomer or hyperoligomeric pilus structures comprising GAS AI-5 surface proteins may be purified or otherwise formulated for use in immunogenic compositions.

GAS AI-5 comprises a series of approximately eight open reading frames encoding for a collection of amino acid sequences comprising surface proteins and sortases (“GAS AI-5 proteins”). GAS AI-5 preferably comprises surface proteins, a srtB sortase, a srtC1 sortase and a rofA divergently transcribed transcriptional regulator.

Specifically, GAS AI-5 includes polynucleotide sequences encoding for two or more of MGAS10270_Spy0108, MGAS10270_Spy0109, MGAS10270_Spy0110, MGAS10270_Spy0111, MGAS10270_Spy0112, MGAS10270_Spy0113, MGAS10270_Spy0114, MGAS10270_Spy0115, MGAS10270_Spy0116, and MGAS10270_Spy0117. One or more of the GAS AI-5 polynucleotide sequences may be replaced by a polynucleotide sequence coding for a fragment of the replaced ORF. Alternatively, one or more of the GAS AI-5 open reading frames may be replaced by a sequence having sequence homology (sequence identity) to the replaced ORF.

One or more of the GAS AI-5 surface proteins typically include an LPXTG motif (e.g., SEQ ID NO:122) or other sortase substrate motif. These sortase proteins are thought to be involved in the secretion and anchoring of the LPXTG containing surface proteins. GAS AI-5 may encode for at least one surface protein. Alternatively, GAS AI-5 may encode for at least two surface proteins and at least one sortase. Preferably, GAS AI-5 encodes for at least three surface proteins and at least two sortases. One or more of the surface proteins may include an LPXTG motif.

GAS AI-5 preferably includes a srtB sortase and a srtC1 sortase. As discussed above, GAS srtB sortases may preferably anchor surface proteins with an LPSTG motif (SEQ ID NO:166), particularly where the motif is followed by a serine. GAS srtC1 sortase may preferentially anchor surface proteins with a V(P/V)PTG (SEQ ID NO:167) motif. GAS srtC1 may be differentially regulated by rofA.

The GAS AI-5 protein of the composition may be selected from the group consisting of MGAS10270_Spy0108, MGAS10270_Spy0109, MGAS10270_Spy0110, MGAS10270_Spy0111, MGAS10270_Spy0112, MGAS10270_Spy0113, MGAS10270_Spy0114, MGAS10270_Spy0115, MGAS10270_Spy0116, and MGAS10270_Spy0117. GAS AI-5 surface proteins are preferred for use in the immunogenic compositions of the invention. Preferably, each of these GAS AI-5 surface proteins includes a sortase substrate motif.

In addition to the open reading frames encoding the GAS AI-5 proteins, GAS AI-5 may also include a divergently transcribed transcriptional regulator such as rofA (i.e., the transcriptional regulator is located near or adjacent to the GAS AI protein open reading frames, but it transcribed in the opposite direction).The GAS AI-5 surface proteins may be used alone, in combination with other GAS AI-5 surface proteins or in combination with other GAS AI surface proteins.

A sixth GAS adhesion island, “GAS Adhesin Island-6” or “GAS AI-6,” has also been identified in GAS serotypes. Amino acid sequences encoded by the open reading frames of GAS AI-6 may also be used in immunogenic compositions for the treatment or prevention of GAS infection.

A preferred immunogenic composition of the invention comprises a GAS AI-6 surface protein which may be formulated or purified in an oligomeric (pilus) form. In a preferred embodiment, the oligomeric form is a hyperoligomer. A preferred immunogenic composition of the invention alternatively comprises an isolated GAS AI-6 surface protein in oligomeric (pilus) form. The oligomer or hyperoligomeric pilus structures comprising GAS AI-6 surface proteins may be purified or otherwise formulated for use in immunogenic compositions.

GAS AI-6 comprises a series of approximately eight open reading frames encoding for a collection of amino acid sequences comprising surface proteins and sortases (“GAS AI-6 proteins”). GAS AI-6 preferably comprises surface proteins, a srtB sortase, a srtC1 sortase and a rofA divergently transcribed transcriptional regulator.

Specifically, GAS AI-6 includes polynucleotide sequences encoding for two or more of MGAS10750_Spy0113, MGAS10750_Spy0114, MGAS10750_Spy0115, MGAS10750_Spy0116, MGAS10750_Spy0117, MGAS10750_Spy0118, MGAS10750_Spy0119, and MGAS10750_Spy0120.

One or more of the GAS AI-6 polynucleotide sequences may be replaced by a polynucleotide sequence coding for a fragment of the replaced ORF. Alternatively, one or more of the GAS AI-6 open reading frames may be replaced by a sequence having sequence homology (sequence identity) to the replaced ORF.

One or more of the GAS AI-6 surface proteins typically include an LPXTG motif (e.g., SEQ ID NO:122) or other sortase substrate motif. These sortase proteins are thought to be involved in the secretion and anchoring of the LPXTG containing surface proteins. GAS AI-6 may encode for at least one surface protein. Alternatively, GAS AI-6 may encode for at least two surface proteins and at least one sortase. Preferably, GAS AI-6 encodes for at least three surface proteins and at least two sortases. One or more of the surface proteins may include an LPXTG motif.

GAS AI-6 preferably includes a srtB sortase and a srtC1 sortase. As discussed above, GAS srtB sortases may preferably anchor surface proteins with an LPSTG motif (SEQ ID NO:166), particularly where the motif is followed by a serine. GAS srtC1 sortase may preferentially anchor surface proteins with a V(P/V)PTG (SEQ ID NO:167) motif. GAS srtC1 may be differentially regulated by rofA.

The GAS AI-6 protein of the composition may be selected from the group consisting of Specifically, GAS AI-6 includes polynucleotide sequences encoding for two or more of MGAS10750_Spy0113, MGAS10750_Spy0114, MGAS10750_Spy0115, MGAS10750_Spy0116, MGAS10750_Spy0117, MGAS10750_Spy0118, MGAS10750_Spy0119, and MGAS10750_Spy0120. GAS AI-6 surface proteins are preferred for use in the immunogenic compositions of the invention. Preferably, each of these GAS AI-6 surface proteins includes a sortase substrate motif.

In addition to the open reading frames encoding the GAS AI-6 proteins, GAS AI-6 may also include a divergently transcribed transcriptional regulator such as rofA (i.e., the transcriptional regulator is located near or adjacent to the GAS AI protein open reading frames, but it transcribed in the opposite direction).The GAS AI-6 surface proteins may be used alone, in combination with other GAS AI-6 surface proteins or in combination with other GAS AI surface proteins.

The GAS AI proteins of the invention may be used in immunogenic compositions for prophylactic or therapeutic immunization against GAS infection. For example, the invention may include an immunogenic composition comprising one or more GAS AI-1 proteins and one or more of any of GAS AI-2, GAS AI-3, or GAS AI-4 proteins. For example, the invention includes an immunogenic composition comprising at least two GAS AI proteins where each protein is selected from a different GAS adhesin island. The two GAS AI proteins may be selected from one of the following GAS AI combinations: GAS AI-1 and GAS AI-2; GAS AI-1 and GAS AI-3; GAS AI-1 and GAS AI-4; GAS AI-2 and GAS AI-3; GAS AI-2 and GAS AI-4; and GAS AI 3 and GAS AI-4. Preferably the combination includes fimbrial proteins from one or more GAS adhesin islands.

The immunogenic compositions may also be selected to provide protection against an increased range of GAS serotypes and strain isolates. For example, the immunogenic composition may comprise a first and second GAS AI protein, wherein a full length polynucleotide sequence encoding for the first GAS AI protein is not present in a genome comprising a full length polynucleotide sequence encoding for the second GAS AI protein. In addition, each antigen selected for use in the immunogenic compositions will preferably be present in the genomes of multiple GAS serotypes and strain isolates. Preferably, each antigen is present in the genomes of at least two (i.e., 3, 4, 5, 6, 7, 8, 9, 10, or more) GAS strain isolates. More preferably, each antigen is present in the genomes of at least two (i.e., at least 3, 4, 5, or more) GAS serotypes.

Applicants have also identified adhesin islands within the genome of Streptococcus pneumoniae. These adhesion islands are thought to encode surface proteins which are important in the bacteria's virulence. Amino acid sequence encoded by such S. pneumoniae Adhesin Islands may be used in immunogenic compositions for the treatment or prevention of S. pneumoniae infection. Preferred immunogenic compositions of the invention comprise a S. pneumoniae AI surface protein which has been formulated or purified in an oligomeric (pilus) form. In a preferred embodiment, the oligomeric form is a hyperoligomer. A preferred immunogenic composition of the invention alternatively comprises an isolated S. pneumoniae surface protein in oligomeric (pilus) form. The oligomer or hyperoligomeric pilus structures comprising S. pneumoniae surface proteins may be purified or otherwise formulated for use in immunogenic compositions.

The S. pneumoniae Adhesin Islands generally include a series of open reading frames within a S. pneumoniae genome that encode for a collection of surface proteins and sortases. A S. pneumoniae Adhesin Island may encode for an amino acid sequence comprising at least one surface protein. Alternatively, the S. pneumoniae Adhesin Island may encode for at least two surface proteins and at least one sortase. Preferably, a S. pneumoniae Adhesin Island encodes for at least three surface proteins and at least two sortases. One or more of the surface proteins may include an LPTXG motif (e.g., SEQ ID NO:122) or other sortase substrate motif. One or more S. pneumoniae AI surface proteins may participate in the formation of a pilus structure on the surface of the S. pneumoniae bacteria.

The S. pneumoniae Adhesin Islands of the invention preferably include a divergently transcribed transcriptional regulator. The transcriptional regulator may regulate the expression of the S. pneumoniae AI operon. An example of a transcriptional regulator found in S. pneumoniae AI sequences is rlrA.

A schematic of the organization of a S. pneumoniae AI locus is provided in FIG. 137. The locus comprises open reading frames encoding a transcriptional regulator (rlrA), cell wall surface proteins (rrgA, rrgB, rrgC) and sortases (srt B, srtC, srtD).

S. pneumoniae AI sequences may be generally divided into two groups of homology, S. pneumoniae AI-a and AI-b. S. pneumoniae strains that comprise AI-a include 14 CSR 10, 19A Hungary 6, 23 F Poland 16, 670, 6B Finland 12, and 6B Spain 2. S. pneumoniae AI strains that comprise AI-b include 19F Taiwan 14, 9V Spain 3, 23F Taiwan 15 and TIGR 4.

S. pneumoniae AI from TIGR4 comprises a series of approximately seven open reading frames encoding for a collection of amino acid sequences comprising surface proteins and sortases (“S. pneumoniae AI proteins”). Specifically, S. pneumoniae AI from TIGR4 includes polynucleotide sequences encoding for two or more of SPO462, SPO463, SPO464, SPO465, SPO466, SPO467, and SPO468.

One or more of the S. pneumoniae AI from TIGR4 polynucleotide sequences may be replaced by a polynucleotide sequence coding for a fragment of the replaced ORF. Alternatively, one or more of the S. pneumoniae AI from TIGR4 open reading frames may be replaced by a sequence having sequence homology to the replaced ORF.

S. pneumoniae strain 670 AI comprises a series of approximately seven open reading frames encoding for a collection of amino acid sequences comprising surface proteins and sortases (“S. pneumoniae AI proteins”). Specifically, S. pneumoniae strain 670 AI includes polynucleotide sequences encoding for two or more of orf1_{—}670, orf3_{—}670, orf4_{—}670, orf5_{—}670, orf6_{—}670, orf7_{—}670, and orf8_{—}670.

One or more of the S. pneumoniae strain 670 AI polynucleotide sequences may be replaced by a polynucleotide sequence coding for a fragment of the replaced ORF. Alternatively, one or more of the S. pneumoniae strain 670 AI open reading frames may be replaced by a sequence having sequence homology to the replaced ORF.

S. pneumoniae AI from 14 CSR10 comprises a series of approximately seven open reading frames encoding for a collection of amino acid sequences comprising surface proteins and sortases (“S. pneumoniae AI proteins”). Specifically, S. pneumoniae AI from 14 CSR10 includes polynucleotide sequences encoding for two or more of ORF2_{—}14CSR, ORF3_{—}14CSR, ORF4_{—}14CSR, ORF5_{—}14CSR, ORF6_{—}14CSR, ORF7_{—}14CSR, and ORF8_{—}14CSR.

One or more of the S. pneumoniae AI from 14 CSR10 polynucleotide sequences may be replaced by a polynucleotide sequence coding for a fragment of the replaced ORF. Alternatively, one or more of the S. pneumoniae AI from 14 CSR10 open reading frames may be replaced by a sequence having sequence homology to the replaced ORF.

S. pneumoniae AI from 19A Hungary 6 comprises a series of approximately seven open reading frames encoding for a collection of amino acid sequences comprising surface proteins and sortases (“S. pneumoniae AI proteins”). Specifically, S. pneumoniae AI from 19A Hungary 6 includes polynucleotide sequences encoding for two or more of ORF2_{—}19AH, ORF3_{—}19AH, ORF4_{—}19AH, ORF5_{—}19AH, ORF6_{—}19AH, ORF7_{—}19AH, and ORF8_{—}19AH.

One or more of the S. pneumoniae AI from 19A Hungary 6 polynucleotide sequences may be replaced by a polynucleotide sequence coding for a fragment of the replaced ORF. Alternatively, one or more of the S. pneumoniae AI from 19A Hungary 6 open reading frames may be replaced by a sequence having sequence homology to the replaced ORF.

S. pneumoniae AI from 19F Taiwan 14 comprises a series of approximately seven open reading frames encoding for a collection of amino acid sequences comprising surface proteins and sortases (“S. pneumoniae AI proteins”). Specifically, S. pneumoniae AI from 19F Taiwan 14 includes polynucleotide sequences encoding for two or more of ORF2_{—}19FTW, ORF3_{—}19FTW, ORF4_{—}19FTW, ORF5_{—}19FTW, ORF6_{—}19FTW, ORF7_{—}19FTW, and ORF8_{—}19FTW.

One or more of the S. pneumoniae AI from 19F Taiwan 14 polynucleotide sequences may be replaced by a polynucleotide sequence coding for a fragment of the replaced ORF. Alternatively, one or more of the S. pneumoniae AI from 19F Taiwan 14 open reading frames may be replaced by a sequence having sequence homology to the replaced ORF.

S. pneumoniae AI from 23F Poland 16 comprises a series of approximately seven open reading frames encoding for a collection of amino acid sequences comprising surface proteins and sortases (“S. pneumoniae AI proteins”). Specifically, S. pneumoniae AI from 23F Poland 16 includes polynucleotide sequences encoding for two or more of ORF2_{—}23FP, ORF3_{—}23FP, ORF4_{—}23FP, ORF5_{—}23FP, ORF6_{—}23FP, ORF7_{—}23FP, and ORF8_{—}23FP.

One or more of the S. pneumoniae AI from 23F Poland 16 polynucleotide sequences may be replaced by a polynucleotide sequence coding for a fragment of the replaced ORF. Alternatively, one or more of the S. pneumoniae AI from 23F Poland 16 open reading frames may be replaced by a sequence having sequence homology to the replaced ORF.

S. pneumoniae AI from 23F Taiwan 15 comprises a series of approximately seven open reading frames encoding for a collection of amino acid sequences comprising surface proteins and sortases (“S. pneumoniae AI proteins”). Specifically, S. pneumoniae AI from 23F Taiwan 15 includes polynucleotide sequences encoding for two or more of ORF2_{—}23FTW, ORF3_{—}23FTW, ORF4_{—}23FTW, ORF5_{—}23FTW, ORF6_{—}23FTW, ORF7_{—}23FTW, and ORF8_{—}23FTW.

One or more of the S. pneumoniae AI from 23F Taiwan 15 polynucleotide sequences may be replaced by a polynucleotide sequence coding for a fragment of the replaced ORF. Alternatively, one or more of the S. pneumoniae AI from 23F Taiwan 15 open reading frames may be replaced by a sequence having sequence homology to the replaced ORF.

S. pneumoniae AI from 6B Finland 12 comprises a series of approximately seven open reading frames encoding for a collection of amino acid sequences comprising surface proteins and sortases (“S. pneumoniae AI proteins”). Specifically, S. pneumoniae AI from 6B Finland 12 includes polynucleotide sequences encoding for two or more of ORF2_{—}6BF, ORF3_{—}6BF, ORF4_{—}6BF, ORF5_{—}6BF, ORF6_{—}6BF, ORF7_{—}6BF, and ORF8_{—}6BF.

One or more of the S. pneumoniae AI from 6B Finland 12 polynucleotide sequences may be replaced by a polynucleotide sequence coding for a fragment of the replaced ORF. Alternatively, one or more of the S. pneumoniae AI from 6B Finland 12 open reading frames may be replaced by a sequence having sequence homology to the replaced ORF.

S. pneumoniae AI from 6B Spain 2 comprises a series of approximately seven open reading frames encoding for a collection of amino acid sequences comprising surface proteins and sortases (“S. pneumoniae AI proteins”). Specifically, S. pneumoniae AI from 6B Spain 2 includes polynucleotide sequences encoding for two or more of ORF2_{—}6BSP, ORF3_{—}6BSP, ORF4_{—}6BSP, ORF5_{—}6BSP, ORF6_{—}6BSP, ORF7_{—}6BSP, and ORF8_{—}6BSP.

One or more of the S. pneumoniae AI from 6B Spain 2 polynucleotide sequences may be replaced by a polynucleotide sequence coding for a fragment of the replaced ORF. Alternatively, one or more of the S. pneumoniae AI from 6B Spain 2 open reading frames may be replaced by a sequence having sequence homology to the replaced ORF.

S. pneumoniae AI from 9V Spain 3 comprises a series of approximately seven open reading frames encoding for a collection of amino acid sequences comprising surface proteins and sortases (“S. pneumoniae AI proteins”). Specifically, S. pneumoniae AI from 9V Spain 3 includes polynucleotide sequences encoding for two or more of ORF2_{—}9VSP, ORF3_{—}9VSP, ORF4_{—}9VSP, ORF5_{—}9VSP, ORF6_{—}9VSP, ORF7_{—}9VSP, and ORF8_{—}9VSP.

One or more of the S. pneumoniae AI from 9V Spain 3 polynucleotide sequences may be replaced by a polynucleotide sequence coding for a fragment of the replaced ORF. Alternatively, one or more of the S. pneumoniae AI from 9V Spain 3 open reading frames may be replaced by a sequence having sequence homology to the replaced ORF.

One or more of the S. pneumoniae AI surface proteins typically include an LPXTG motif (e.g., SEQ ID NO:122) or other sortase substrate motif. These sortase proteins are thought to be involved in the secretion and anchoring of the LPXTG containing surface proteins. S. pneumoniae AI may encode for at least one surface protein. The Adhesin Island, may encode at least one surface protein. Alternatively, S. pneumoniae AI may encode for at least two surface proteins and at least one sortase. Preferably, S. pneumoniae AI encodes for at least three surface proteins and at least two sortases. One or more of the surface proteins may include an LPXTG motif.

The S. pneumoniae AI protein of the composition may be selected from the group consisting of SPO462, SPO463, SPO464, SPO465, SPO466, SPO467, SPO468, orf1_{—}670, orf3_{—}670, orf4_{—}670, orf5_{—}670, orf6_{—}670, orf7_{—}670, orf8_{—}670, ORF2_{—}14CSR, ORF3_{—}14CSR, ORF4_{—}14CSR, ORF5_{—}14CSR, ORF6_{—}14CSR, ORF7_{—}14CSR, ORF8_{—}14CSR, ORF2_{—}19AH, ORF3_{—}19AH, ORF4_{—}19AH, ORF5_{—}19AH, ORF6_{—}19AH, ORF7_{—}19AH, ORF8_{—}19AH, ORF2_{—}19FTW, ORF3_{—}19FTW, ORF4_{—}19FTW, ORF5_{—}19FTW, ORF6_{—}19FTW, ORF7_{—}19FTW, ORF8_{—}19FTW, ORF2_{—}23FP, ORF3_{—}23FP, ORF4_{—}23FP, ORF5_{—}23FP, ORF6_{—}23FP, ORF7_{—}23FP, ORF8_{—}23FP, ORF2_{—}23FTW, ORF3_{—}23FTW, ORF4_{—}23FTW, ORF5_{—}23FTW, ORF6_{—}23FTW, ORF7_{—}23FTW, ORF8_{—}23FTW, ORF2_{—}6BF, ORF3_{—}6BF, ORF4_{—}6BF, ORF5_{—}6BF, ORF6_{—}6BF, ORF7_{—}6BF, ORF8_{—}6BF, ORF2_{—}6BSP, ORF3_{—}6BSP, ORF4_{—}6BSP, ORF5_{—}6BSP, ORF6_{—}6BSP, ORF7_{—}6BSP, ORF8_{—}6BSP, ORF2_{—}9VSP, ORF3_{—}9VSP, ORF4_{—}9VSP, ORF5_{—}9VSP, ORF6_{—}9VSP, ORF7_{—}9VSP and, ORF8_{—}9VSP.

S. pneumoniae AI surface proteins are preferred proteins for use in the immunogenic compositions of the invention. In one embodiment, the compositions of the invention comprise combinations of two or more S pneumoniae AI surface proteins. Preferably such combinations are selected from two or more of the group consisting of SPO462, SPO463, SPO464, orf3_{—}670, orf4_{—}670, orf5_{—}670, ORF3_{—}14CSR, ORF4_{—}14CSR, ORF5_{—}14CSR, ORF3_{—}19AH, ORF4_{—}19AH, ORF5_{—}19AH, ORF3_{—}19FTW, ORF4_{—}19FTW, ORF5_{—}19FTW, ORF3_{—}23FP, ORF4_{—}23FP, ORF5_{—}23FP, ORF3_{—}23FTW, ORF4_{—}23FTW, ORF5_{—}23FTW, ORF3_{—}6BF, ORF4_{—}6BF, ORF5_{—}6BF, ORF3_{—}6BSP, ORF4_{—}6BSP, ORF5_{—}6BSP, ORF3_{—}9VSP, ORF4_{—}9VSP, and ORF5_{—}9VSP.

In addition to the open reading frames encoding the S. pneumoniae AI proteins, S. pneumoniae AI may also include a transcriptional regulator.

The S. pneumoniae AI proteins of the invention may be used in immunogenic compositions for prophylactic or therapeutic immunization against S. pneumoniae infection. For example, the invention may include an immunogenic composition comprising one or more S. pneumoniae from TIGR4 AI proteins and one or more S. pneumoniae strain 670 proteins. The immunogenic composition may comprise one or more AI proteins from any one or more of S. pneumoniae strains TIGR4, 19A Hungary 6, 6B Finland 12, 6B Spain 2, 9V Spain 3, 14 CSR 10, 19F Taiwan 14, 23F Taiwan 15, 23F Poland 16, and 670.

The immunogenic compositions may also be selected to provide protection against an increased range of S. pneumoniae serotypes and strain isolates. For example, the immunogenic composition may comprise a first and second S. pneumoniae AI protein, wherein a full length polynucleotide sequence encoding for the first S. pneumoniae AI protein is not present in a genome comprising a full length polynucleotide sequence encoding for the second S. pneumoniae AI protein. In addition, each antigen selected for use in the immunogenic compositions will preferably be present in the genomes of multiple S. pneumoniae serotypes and strain isolates. Preferably, each antigen is present in the genomes of at least two (i.e., 3, 4, 5, 6, 7, 8, 9, 10, or more) S. pneumoniae strain isolates. More preferably, each antigen is present in the genomes of at least two (i.e., at least 3, 4, 5, or more) S. pneumoniae serotypes.

The immunogenic compositions may also be selected to provide protection against an increased range of serotypes and strain isolates of a Gram positive bacteria. For example, the immunogenic composition may comprise a first and second Gram positive bacteria AI protein, wherein a full length polynucleotide sequence encoding for the first Gram positive bacteria AI protein is not present in a genome comprising a full length polynucleotide sequence encoding for the second Gram positive bacteria AI protein. In addition, each antigen selected for use in the immunogenic compositions will preferably be present in the genomes of multiple serotypes and strain isolates of the Gram positive bacteria. Preferably, each antigen is present in the genomes of at least two (i.e., 3, 4, 5, 6, 7, 8, 9, 10, or more) Gram positive bacteria strain isolates. More preferably, each antigen is present in the genomes of at least two (i.e., at least 3, 4, 5, or more) Gram positive bacteria serotypes. One or both of the first and second AI proteins may preferably be in oligomeric or hyperoligomeric form.

Adhesin island surface proteins from two or more Gram positive bacterial genus or species may be combined to provide an immunogenic composition for prophylactic or therapeutic treatment of disease or infection of two more Gram positive bacterial genus or species. Optionally, the adhesin island surface proteins may be associated together in an oligomeric or hyperoligomeric structure.

In one embodiment, the invention comprises adhesin island surface proteins from two or more Streptococcus species. For example, the invention includes a composition comprising a GBS AI surface protein and a GAS adhesin island surface protein. As another example, the invention includes a composition comprising a GAS adhesin island surface protein and a S. pneumoniae adhesin island surface protein. One or both of the GAS AI surface protein and the S. pneumoniae AI surface protein may be in oligomeric or hyperoligomeric form. As a further example, the invention includes a composition comprising a GBS adhesin island surface protein and a S. pneumoniae adhesin island surface protein.

In one embodiment, the invention comprises an adhesin island surface protein from two or more Gram positive bacterial genus. For example, the invention includes a composition comprising a Streptococcus adhesin island protein and a Corynebacterium adhesin island protein. One or more of the Gram positive bacteria AI surface proteins may be in an oligomeric or hyperoligomeric form.

In addition, the AI polynucleotides and amino acid sequences of the invention may also be used in diagnostics to identify the presence or absence of GBS (or a Gram positive bacteria) in a biological sample. They may be used to generate antibodies which can be used to identify the presence of absence of an AI protein in a biological sample or in a prophylactic or therapeutic treatment for GBS (or a Gram positive bacterial) infection. Further, the AI polynucleotides and amino acid sequences of the invention may also be used to identify small molecule compounds which inhibit or decrease the virulence associated activity of the AI.

In certain preferred aspects, the invention comprises three antigens wherein each antigen is selected from a different adhesin island AI-1 (PI-1), AI2 subgroup 1 (PI-2a), and AI2 subgroup 2 (PI-2b). In preferred embodiments, the antigen from AI-1 is the backbone pilin antigen (GBS80 or variants thereof). In preferred embodiments, the antigen from AI-2 subgroup 1 is the anciliary pilin 1 antigen (GBS67 or variants thereof). In preferred embodiments, the antigen from AI-2 subgroup 2 is the backbone pilin antigen. In preferred embodiments, the three antigens are in a vaccine composition that may be used to provoke an antibody response in a mammal or for providing broad range protection against GBS infection in a mammal (in each case preferably a human). The antigens may be in any form as disclosed throughout this specification (e.g., full length, fragments that are antigenic, immunogenic or otherwise can be bound by an antibody that binds the naturally occurring full length antigen from which they are derived). The three antigens may also be used in the preparation of medicaments as disclosed throughout this specification. As discussed more fully below, the vaccine and medicaments may further comprise an adjuvant. The various compositions including these three antigens may be used in the methods and for the uses as disclosed further below (e.g., methods of administration).

### BRIEF DESCRIPTION OF THE FIGURES

FIG. 1 presents a schematic depiction of GBS Adhesin Island 1 (“AI-1”) comprising open reading frames for GBS 80, GBS 52, SAG0647, SAG0648 and GBS 104.

FIG. 2 illustrates the identification of AI-1 sequences in several GBS serotypes and strain isolates (GBS serotype V, strain isolate 2603; GBS serotype III, strain isolate nem316; GBS serotype II, strain isolate 18RS21; GBS serotype V, strain isolate CJB111; GBS serotype III, strain isolate COH1 and GBS serotype 1a, strain isolate A909). (An AI-1 was not identified in GBS serotype 1b, strain isolate H36B or GBS serotype 1a, strain isolate 515).

FIG. 3 presents a schematic depiction of the correlation between AI-1 and the Adhesin Island 2 (“AI-2”) within the GBS serotype V, strain isolate 2603 genome. (This AI-2 comprises open reading frames for GBS 67, GBS 59, SAG1406, SAG1405 and GBS 150).

FIG. 4 illustrates the identification of AI-2 comprising open reading frames encoding for GBS 67, GBS 59, SAG1406, SAG1404 and GBS 150 (or sequences having sequence homology thereto) in several GBS serotypes and strain isolates (GBS serotype V, strain isolate 2603; GBS serotype III, strain isolate NEM316; GBS serotype 1b, strain isolate H36B; GBS serotype V, strain isolate CJB111; GBS serotype II, strain isolate 18RS21; and GBS serotype 1a, strain isolate 515). FIG. 4 also illustrates the identification of AI-2 comprising open reading frames encoding for 01520 (a sortase), 01521, 01522 (a sortase), 01523 (spb1), 01524 and 01525 (or sequences having sequence homology thereto).

FIG. 5 presents data showing that GBS 80 binds to fibronectin and fibrinogen in ELISA.

FIG. 6 illustrates that all genes in AI-1 are co-transcribed as an operon.

FIG. 7 presents schematic depictions of in-frame deletion mutations within AI-1.

FIG. 8 presents FACS data showing that GBS 80 is required for surface localization of GBS 104.

FIG. 9 presents FACS data showing that sortases SAG0647 and SAG0648 play a semi-redundant role in surface exposure of GBS 80 and GBS 104.

FIG. 10 presents Western Blots of the in-frame deletion mutants probed with anti-GBS80 and anti-GBS 104 antisera.

FIG. 11: Electron micrograph of surface exposed pili structures in Streptococcus agalactiae containing GBS 80.

FIG. 12: PHD predicted secondary structure of GBS 067.

FIGS. 13, 14 and 15: Electron micrographs of surface exposed pili structures of strain isolate COH1 of Streptococcus agalactiae containing a plasmid insert encoding GBS 80.

FIGS. 16 and 17: Electron micrographs of surface exposed pili structure of wild type strain isolate COH1 of Streptococcus agalactiae.

FIG. 18: Alignment of polynucleotide sequences of AI-1 from serotype V, strain isolates 2603 and CJB111; serotype II, strain isolate 8 RS21; serotype strain isolates COH1 and NEM316; and serotype 1a, strain isolate A909.

FIG. 19: Alignment of polynucleotide sequences of AI-2 from serotype V, strain isolates 2603 and CJB111; serotype II, strain isolate 18RS21; serotype 1b, strain isolate H36B; and serotype 1a, strain isolate 515.

FIG. 20: Alignment of polynucleotide sequences of AI-2 from serotype V, strain isolate 2603 and serotype III, strain isolate NEM316.

FIG. 21: Alignment of polynucleotide sequences of AI-2 from serotype III, strain isolate COH1 and serotype Ia, strain isolate A909.

FIG. 22: Alignment of amino acid sequences of AI-1 surface protein GBS 80 from serotype V, strain isolates 2603 and CJB111; serotype 1a, strain isolate A909; serotype III, strain isolates COH1 and NEM316.

FIG. 23: Alignment of amino acid sequences of AI-1 surface protein GBS 104 from serotype V, strain isolates 2603 and CJB111; serotype III, strain isolates COH1 and NEM316; and serotype II, strain isolate 18RS21.

FIG. 24: Alignment of amino acid sequences of AI-2 surface protein GBS 067 from serotype V, strain isolates 2603 and CJB 111; serotype 1a, strain isolate 515; serotype II, strain isolate 18RS21; serotype Ib, strain isolate H36B; and serotype III, strain solate NEM316.

FIG. 25: Illustrates that GBS closely associates with tight junctions and cross the monolayer of ME180 cervical epithelial cells by a paracellular route.

FIG. 26: Illustrates GBS infection of ME180 cells.

FIG. 27: Illustrates that GBS 80 recombinant protein does not bind to epithelial cells.

FIG. 28: Illustrates that deletion of GBS 80 does not effect the capacity of GBS strain 2603 V/R to adhere and invade ME180 cervical epithelial cells.

FIG. 29: Illustrates binding of recombinant GBS 104 protein to epithelial cells.

FIG. 30: Illustrates that deletion of GBS 104 in the GBS strain COH1, reduces the capacity of GBS to adhere to ME180 cervical epithelial cells.

FIG. 31: Illustrates that GBS 80 knockout mutant strain partially loses the ability to translocate through an epithelial cell monolayer.

FIG. 32: Illustrates that deletion of GBS 104, but not GBS 80, reduces the capacity of GBS to invade J774 macrophage-like cell line.

FIG. 33: Illustrates that GBS 104 knockout mutant strain translocates through an epithelial monolayer less efficiently than the isogenic wild type.

FIG. 34: Negative stained electron micrographs of GBS serotype III, strain isolate COH1, containing a plasmid insert to over-express GBS 80.

FIG. 35: Electron micrographs of surface exposed pili structures on GBS serotype III, strain isolate COH1, containing a plasmid insert to over-express GBS 80, stained with anti-GBS 80 antibodies (visualized with 10 nm gold particles).

FIG. 36: Electron micrographs of surface exposed pili structures on GBS serotype III, strain isolate COH1, containing a plasmid insert to over-express GBS 80, stained with anti-GBS 80 antibodies (visualized with 10 nm gold particles).

FIG. 37: Electron micrographs of surface exposed pili structures on GBS serotype III, strain isolate COH1, containing a plasmid insert to over-express GBS 80, stained with anti-GBS 80 antibodies (visualized with 20 n m gold particles).

FIG. 38: Electron micrographs of surface exposed pili structures on GBS serotype III, strain isolate COH1, containing a plasmid insert to over-express GBS 80, stained with anti-GBS 104 antibodies or preimmune sera (visualized with 10 nm gold particles).

FIG. 39: Electron micrographs of surface exposed pili structures on GBS serotype III, strain isolate COH1, containing a plasmid insert to over-express GBS 80, stained with anti-GBS 80 antibodies (visualized with 20 nm gold particles) and anti-GBS 104 antibodies (visualized with 10 nm gold particles).

FIG. 40: Electron micrographs of surface exposed pili structures on GBS serotype III, strain isolate COH1, containing a plasmid insert to over-express GBS 80, stained with anti-GBS 80 antibodies (visualized with 20 nm gold particles) and anti-GBS 104 antibodies (visualized with 10 nm gold particles).

FIG. 41: Illustrates that GBS 80 is necessary for polymer formation and GBS104 and sortase SAG0648 are necessary for efficient assembly of pili.

FIG. 42: Illustrates that GBS 67 is part of a second pilus and that GBS 80 is polymerized in strain 515.

FIG. 43: Illustrates that two macro-molecules are visible in Cohl, one of which is the GBS 80 pilin.

FIG. 44: Illustrates pilin assembly.

FIG. 45: Illustrates that GBS 52 is a minor component of the GBS pilus.

FIG. 46: Illustrates that the pilus is found in the supernatant of a bacterial culture.

FIG. 47: Illustrates that the pilus is found in the supernatant of bacterial cultures in all phases.

FIG. 48: Illustrates that in Cohl, only the GBS 80 protein and one sortase (sag0647 or sag0648) is required for polymerization.

FIG. 49: IEM image of GBS 80 staining of a GBS serotype VIII strain JM9030013 that express pili.

FIG. 50: IEM image of GBS 104 staining of a GBS serotype VIII strain JM9030013 that express pili.

FIG. 51A: Schematic depiction of open reading frames comprising GAS AI-2 serotype M1 isolate, GAS AI-3 serotype M3, M5, M18, and M49 isolates, a GAS AI-4 serotype M12 isolate, and an GAS AI-1 serotype M6 isolate.

FIG. 51B: Amino acid alignment of SrtC1-type sortase of a GAS AI-2 serotype M1 isolate, SrtC2-type sortases of serotype M3, M5, M18, and M49 isolates, and a SrtC2-type sortase of a GAS AI-4 serotype M12 isolate.

FIG. 52: Amino acid alignment of the capsular polysacharide adhesion proteins of GAS AI-4 serotype M12 (A735), GAS AI-3 serotype M5 (Manfredo), S. pyogenes strain MGAS315 serotype M3, S. pyogenes strain SSI-1 serotype M3, S. pyogenes strain MGAS8232 serotype M3, and GAS AI-2 serotype M1.

FIG. 53: Amino acid alignment of F-like fibronectin-binding roteins of GAS AI-4 serotype M12 (A735) and S. pyogenes strain MGAS10394 serotype M6.

FIG. 54: Amino acid alignment of F2-like fibronectin-binding proteins of GAS AI-4 serotype M12 (A735), S. pyogenes strain MGAS8232 serotype M3, GAS AI-3 strain M5 (Manfredo), S. pyogenes strain SSI serotype M3, and S. pyogenes stain MGAS315 serotype M3.

FIG. 55: Amino acid alignment of fimbrial proteins of GAS AI-4 serotype M12 (A735), GAS AI-3 serotype M5 (Manfredo), S. pyogenes strain MGAS315 serotype M3, S. pyogenes strain SSI serotype M3, S. pyogenes strain MGAS8232 serotype M3, and S. pyogenes M1 GAS serotype M1.

FIG. 56: Amino acid alignment of hypothetical proteins of GAS AI-4 serotype M12 (A735), S. pyogenes strain MGAS315 serotype M3, S. pyogenes strain SSI-1 serotype M3, GAS AI-3 serotype M5 (Manfredo), and S. pyogenes strain MGAS8232 serotype M3.

FIG. 57: Results of FASTA homology search for amino acid sequences that align with the collagen adhesion protein of GAS AI-1 serotype M6 (MGAS10394).

FIG. 58: Results of FASTA homology search for amino acid sequences that align with the fimbrial structural subunit of GAS AI-1 serotype M6 (MGAS10394).

FIG. 59: Results of FASTA homology search for amino acid sequences that align with the hypothetical protein of GAS AI-2 serotype M1 (SF370).

FIG. 60: Specifies pilin and E box motifs present in GAS type 3 and 4 adhesin islands.

FIG. 61: Illustrates that surface expression of GBS 80 protein on GBS strains COH and JM9130013 correlates with formation of pili structures. Surface expression of GBS 80 was determined by FACS analysis using an antibody that cross-hybridizes with GBS 80. Formation of pili structures was determined by immunogold electron microscopy using gold-labelled anti-GBS 80 antibody.

FIG. 62: Illustrates that surface exposure is capsule-dependent for GBS 322 but not for GBS 80.

FIG. 63: Illustrates the amino acid sequence identity of GBS 59 proteins in GBS strains.

FIG. 64: Western blotting of whole GBS cell extracts with anti-GBS 59 antibodies.

FIG. 65: Western blotting of purified GBS 59 and whole GBS cell extracts with anti-GBS 59 antibodies.

FIG. 66: FACS analysis of GBS strains CJB111, 7357B, 515 using GBS 59 antiserum.

FIG. 67: Illustrates that anti-GBS 59 antibodies are opsonic for CJB111 GBS strain serotype V.

FIG. 68: Western blotting of GBS strain JM9130013 total extracts.

FIG. 69: Western blotting of GBS stain 515 total extracts shows that GBS 67 and GBS 150 are parts of a pilus.

FIG. 70: Western blotting of GBS strain 515 knocked out for GBS 67 expression

FIG. 71: FACS analysis of GBS strain 515 and GBS strain 515 knocked out for GBS 67 expression using GBS 67 and GBS 59 antiserum.

FIG. 72: Illustrates complementation of GBS 515 knocked out for GBS 67 expression with a construct overexpressing GBS 80.

FIG. 73: FACS analysis of GAS serotype M6 for spyM6_{—}0159 surface expression.

FIG. 74: FACS analysis of GAS serotype M6 for spyM6_{—}0160 surface expression.

FIG. 75: FACS analysis of GAS serotype M1 for GAS 15 surface expression.

FIG. 76: FACS analysis of GAS serotype M1 for GAS 16 surface expression using a first anti-GAS 16 antiserum.

FIG. 77: FACS analysis of GAS serotype M1 for GAS 18 surface expression using a first anti-GAS 18 antiserum.

FIG. 78: FACS analysis of GAS serotype M1 for GAS 18 surface expression using a second anti-GAS 18 antiserum.

FIG. 79: FACS analysis of GAS serotype M1 for GAS 16 surface expression using a second anti-GAS 16 antisera.

FIG. 80: FACS analysis of GAS serotype M3 for spyM3_{—}0098 surface expression.

FIG. 81: FACS analysis of GAS serotype M3 for spyM3_{—}0100 surface expression.

FIG. 82: FACS analysis of GAS serotype M3 for spyM3_{—}0102 surface expression.

FIG. 83: FACS analysis of GAS serotype M3 for spyM3_{—}0104 surface expression.

FIG. 84: FACS analysis of GAS serotype M3 for spyM3_{—}0106 surface expression.

FIG. 85: FACS analysis of GAS serotype M12 for 19224134 surface expression.

FIG. 86: FACS analysis of GAS serotype M12 for 19224135 surface expression.

FIG. 87: FACS analysis of GAS serotype M12 for 19224137 surface expression.

FIG. 88: FACS analysis of GAS serotype M12 for 19224141 surface expression.

FIG. 89: Western blot analysis of GAS 15 expression on GAS M1 bacteria.

FIG. 90: Western blot analysis of GAS 15 expression using GAS 15 immune sera.

FIG. 91: Western blot analysis of GAS 15 expression using GAS 15 pre-immune sera.

FIG. 92: Western blot analysis of GAS 16 expression on GAS M1 bacteria.

FIG. 93: Western blot analysis of GAS 16 expression using GAS 16 immune sera.

FIG. 94: Western blot analysis of GAS 16 expression using GAS 16 pre-immune sera.

FIG. 95: Western blot analysis of GAS 18 on GAS M1 bacteria.

FIG. 96: Western blot analysis of GAS 18 using GAS 18 immune sera.

FIG. 97: Western blot analysis of GAS 18 using GAS 18 pre-immune sera.

FIG. 98: Western blot analysis of M6_Spy0159 expression on GAS bacteria.

FIG. 99: Western blot analysis of 19224135 expression on M12 GAS bacteria.

FIG. 100: Western blot analysis of 19224137 expression on M12 GAS bacteria.

FIG. 101: Full length nucleotide sequence of an S. pneumoniae strain 670 AI.

FIG. 102: Western blot analysis of GAS 15, GAS 16, and GAS 18 in GAS M1 strain 2580.

FIG. 103: Western blot analysis of GAS 15, GAS 16, and GAS 18 in GAS M1 strain 2913.

FIG. 104: Western blot analysis of GAS 15, GAS 16, and GAS 18 in GAS M1 strain 3280.

FIG. 105: Western blot analysis of GAS 15, GAS 16, and GAS 18 in GAS M1 strain 3348.

FIG. 106: Western blot analysis of GAS 15, GAS 16, and GAS 18 in GAS M1 strain 2719.

FIG. 107: Western blot analysis of GAS 15, GAS 16, and GAS 18 in GAS M1 strain SF370.

FIG. 108: Western blot analysis of 19224135 and 19224137 in GAS M12 strain 2728.

FIG. 109: Western blot analysis of 19224139 in GAS M12 strain 2728 using antisera raised against SpyM3_{—}0102.

FIG. 110: Western blot analysis of M6_Spy0159 and M6_Spy0160 in GAS M6 strain 2724.

FIG. 111: Western blot analysis of M6_Spy0159 and M6_Spy0160 in GAS M6 strain SF370.

FIG. 112: Western blot analysis of M6_Spy160 in GAS M6 strain 2724.

FIGS. 113-115: Electron micrographs of surface exposed GAS 15 on GAS M1 strain SF370.

FIGS. 116-121: Electron micrographs of surface exposed GAS 16 on GAS M1 strain SF370.

FIGS. 122-125: Electron micrographs of surface exposed GAS 18 on GAS M1 strain SF370 detected using anti-GAS 18 antisera.

FIG. 126: IEM image of a hyperoligomer on GAS M1 strain SF370 detected using anti-GAS 18 antisera.

FIGS. 127-132: IEM images of oligomeric and hyperoligomeric structures containing M6_Spy0160 extending from the surface of GAS serotype M6 3650.

FIGS. 133A and B: Western blot analysis of L. lactis transformed to express GBS 80 with anti-GBS 80 antiserum.

FIGS. 134: Western blot analyses of L. lactis transformed to express GBS AI-1 with anti-GBS 80 antiserum.

FIG. 135: Ponceau staining of same acrylamide gel as used in FIG. 134.

FIG. 136A: Western blot analysis of sonicated pellets and supernatants of cultured L. lactis transformed to express GBS AI-1 polypeptides using anti-GBS 80 antiserum.

FIG. 136B: Polyacrylamide gel electrophoresis of sonicated pellets and supernatants of cultured L. lactis transformed to express GBS AI polypeptides.

FIG. 137: Depiction of an example S. pneumoniae AI locus.

FIG. 138: Schematic of primer hybridization sites within the S. pneumoniae AI locus of FIG. 137.

FIG. 139A: The set of amplicons produced from the S. pneumoniae strain TIGR4 AI locus.

FIG. 139B: Base pair lengths of amplicons produced from FIG. 139A primers in S. pneumoniae strain TIGR4.

FIG. 140: CGH analysis of S. pneumoniae strains for the AI locus.

FIG. 141: Amino acid sequence alignment of polypeptides encoded by AI orf 2 in S. pneumoniae AI-positive strain.

FIG. 142: Amino acid sequence alignment of polypeptides encoded by AI orf 3 in S. pneumoniae AI-positive strains.

FIG. 143: Amino acid sequence alignment of polypeptides encoded by AI orf 4 in S. pneumoniae Al-positive strains.

FIG. 144: Amino acid sequence alignment of polypeptides encoded by AI orf 5 in S. pneumoniae AI-positive strains.

FIG. 145: Amino acid sequence alignment of polypeptides encoded by AI orf 6 in S. pneumoniae AI-positive strains.

FIG. 146: Amino acid sequence alignment of polypeptides encoded by AI orf 7 in S. pneumoniae AI-positive strains.

FIG. 147: Amino acid sequence alignment of polypeptides encoded by AI orf 8 in S. pneumoniae Al-positive strains.

FIG. 148: Diagram comparing amino acid sequences of RrgA in S. pneumoniae strains.

FIG. 149: Amino acid sequence comparison of RrgB S. pneumoniae strains.

FIG. 150A: Sp0462 amino acid sequence.

FIG. 150B: Primers used to produce a clone encoding the Sp0462 polypepide.

FIG. 151A: Schematic depiction of recombinant Sp0462 polypeptide.

FIG. 151B: Schematic depiction of full-length Sp0462 polypeptide.

FIG. 152A: Western blot probed with serum obtained from S. pneumoniae-infected patients for Sp0462.

FIG. 152B: Western blot probed with GBS 80 serum for Sp0462.

FIG. 153A: Sp0463 amino acid sequence.

FIG. 153B: Primers used to produce a clone encoding the Sp0463 polypeptide.

FIG. 154A: Schematic depiction of recombinant Sp0463 polypeptide.

FIG. 154B: Schematic depiction of full-length Sp0463 polypeptide.

FIG. 155: Western blot detection of recombinant Sp0463 polypeptide.

FIG. 156: Western blot detection of high molecular weight Sp0463 polymers.

FIG. 157A: Sp0464 amino acid sequence.

FIG. 157B: Primers used to produce a clone encoding the Sp0464 polypeptide.

FIG. 158A: Schematic depiction of recombinant Sp0464 polypeptide.

FIG. 158B: Schematic depiction of full-length Sp0464 polypeptide.

FIG. 159: Western blot detection of recombinant Sp0464 polypeptide.

FIG. 160: Amplification products prepared for production of Sp0462, Sp0463, and Sp0464 clones.

FIG. 161: Opsonic killing by anti-sera raised against L. lactis expressing GBS AI

FIG. 162: Schematic depicting GAS adhesin islands GAS AI-1, GAS AI-2, GAS AI-3 and GAS AI-4.

FIGS. 163A-D: Immunoblots of cell-wall fractions of GAS strains with antisera specific for LPXTG proteins of M6_ISS3650 (A), M1_SF370 (B), M5_ISS4883 (C) and M12_{—}20010296 (D).

FIGS. 163 E-H: Immunoblots of cell-wall fractions of deletion mutants M1_SF370Δ128 (E) M1_SF370Δ130 (F) M1_SF370ΔSrtC1 (G) and the M1_{—}128 deletion strain complemented with plasmid pAM::128 which contains the M1_{—}128 gene (H) with antisera specific for the pilin components of M1_SF370.

FIGS. 163 I-N: Immunogold labelling and transmission electron microscopy of: T6 (I) and Cpa (J) in M6_ISS3650; M1_{—}128 in M1_SF370 (K) and deletion strain M1_SF370Δ128 (N); M5_orf80 in M5_ISS4883 (L); M12_EftLSEA in M12_{—}20010296 (M). The strains used are indicated below the panels. Bars=200 nm.

FIG. 164: Schematic representation of the FCT region from 7 GAS strains

FIGS. 165A-H: Flow cytometry of GAS bacteria treated or not with trypsin and stained with sera specific for the major pilus component. Preimmune staining; black lines, untreated bacteria; green lines and trypsin treated bacteria; blue lines. M6_{—}1553650 stained with sera which recognize the M6 protein (A) or anti-M6_T6 (B), M1_SF370 stained with anti-M1 (C) or anti-M1_{—}128 (D), M5_ISS4883 stained with anti-PrtF (E) or anti-M5_orf80 (F) and M12_{—}20010296 with anti-M12 (G) or anti-EftLSL.A (H)

FIGS. 166A-C: Immunoblots of recombinant pilin components with polyvalent Lancefield T-typing sera. The recombinant proteins are shown above the blot and the sera pool used is shown below the blot.

FIGS. 166 D-G: Immunoblots of pilin proteins with monovalent T-typing sera. The recombinant proteins are shown below the blot and the sera used above the blot.

FIG. 166 H and I Flow cytometry analysis of strain M1_SF370 (H) and the deletion strain M1_SF370Δ128 (I) with T-typing antisera pool T.

FIG. 167: Chart describing the number and type of sortase sequences identified within GAS AIs.

FIG. 168A: Immunogold-electronmicroscopy of L. lactis lacking an expression construct for GBS AI-1 using anti-GBS 80 antibodies.

FIGS. 168B and C: Immunogold-electronmicroscopy detects GBS 80 in oligomeric (pilus) structures on surface of L. lactis transformed to express GBS AI-1

FIG. 169: FACS analysis detects expression of GBS 80 and GBS 104 on the surface of L. lactis transformed to express GBS AI-1.

FIG. 170: Phase contrast microscopy and immuno-electronmicroscopy shows that expression of GBS AI-1 in L. lactis induces L. lactis aggregation.

FIG. 171: Purification of GBS pili from L. lactis transformed to express GBS AI-1.

FIG. 172: Schematic depiction of GAS M6 (AI-1), M1 (AI-2), and M12 (AI-4) adhesin islands and portions of the adhesin islands inserted in the pAM401 construct for expression in L. lactis.

FIG. 173A-C: Western blot analysis showing assembly of GAS pili in L. lactis expressing GAS AI-2 (M1) (A), GAS AI-4 (M12) (B), and GAS AI-1 (M6) (C).

FIG. 174: FACS analysis of GAS serotype M6 for M6_Spy0157 surface expression.

FIG. 175: FACS analysis of GAS serotype M12 for 19224139 surface expression.

FIG. 176A-E: Immunogold electron microscopy using antibodies against M6_Spy0160 detects pili on the surface of M6 strain 2724.

FIG. 176 F: Immunogold electron microscopy using antibodies against M6_Spy0159 detects M6_Spy0159 surface expression on M6 strain 2724.

FIG. 177A-C: Western blot analysis of M1 strain SF370 GAS bacteria individually deleted for M1_{—}130, SrtC1, or M1_{—}128 using anti-M1_{—}130 serum (A), anti-M1_{—}128 serum (B), and anti-M1_{—}126 serum (C).

FIG. 178A-C: Immunogold electron microscopy using antibodies against M1_{—}128 to detect surface expression on wildtype strain SF370 bacteria (A), M1_{—}128 deleted SF370 bacteria (B), and SrtC1 deleted SF370 bacteria (C).

FIG. 179A-C: FACS analysis to detect expression of M1_{—}126 (A), M1_{—}128 (B), and M1_{—}130 (C) on the surface of wildtype SF370 GAS bacteria.

FIG. 179 D-F: FACS analysis to detect expression of M1_{—}126 (D), M1_{—}128 (E), and M1_{—}130 (F) on the surface of M1_{—}128 deleted SF370 GAS bacteria.

FIG. 179 G-I: FACS analysis to detect expression of M1_{—}126 (G), M1_{—}128 (H), and M1_{—}130 (I) on the surface of SrtC1 deleted SF370 GAS bacteria.

FIG. 180A and B: FACS analysis of wildtype (A) and LepA deletion mutant (B) strains of SF370 bacteria for M1 surface expression.

FIG. 181: Western blot analysis detects high molecular weight polymers in S. pneumoniae TIGR4 using anti-RrgB antisera.

FIG. 182: Detection of high molecular weight polymers in S. pneumoniae rlrA positive strains.

FIG. 183: Detection of high molecular weight polymers in S. pneumoniae TIGR4 by silver staining and Western blot analysis using anti-RrgB antisera.

FIG. 184: Deletion of S. pneumoniae TIGR4 adhesin island sequences interferes with the ability of S. pneumoniae to adhere to A549 alveolar cells.

FIG. 185: Negative staining of S. pneumoniae strain TIGR4 showing abundant pili on the bacterial surface.

FIG. 186: Negative staining of strain TIGR4 deleted for rrgA-srtD adhesin island sequences showing no pili on the bacterial surface

FIG. 187: Negative staining of the TIGR4 mgrA mutant showing abundant pili on the bacterial surface.

FIG. 188: Negative staining of the negative control TIGR4 mgrA mutant deleted for adhesin island sequences rrgA-srtD showing no pili on the bacterial surface.

FIG. 189: Immuno-gold labelling of S. pneumoniae strain TIGR4 grown on blood agar solid medium using α-RrgB (5 nm) and α-RrgC (10 nm). Bar represents 200 nm.

FIGS. 190A and B: Detection of expression and purification of S. pneumoniae RrgA protein by SDS-PAGE (A) and Western blot analysis (B).

FIG. 191: Detection of RrgB by antibodies produced in mice.

FIG. 192: Detection of RrgC by antibodies produced in mice.

FIG. 193: Purification of S. pneumoniae TIGR 4 pili by a cultivation and digestion method and detection of the purified TIGR4 pili.

FIG. 194: Purification of S. pneumoniae TIGR 4 pili by a sucrose gradient centrifugation method and detection of the purified TIGR4 pili.

FIG. 195: Purification of S. pneumoniae TIGR 4 pili by a gel filtration method and detection of the purified TIGR4 pili.

FIG. 196: Alignment of full length S. pneumoniae adhesin island sequences S. pneumoniae strains.

FIG. 197A: Schematic of GBS AI-1 coding sequences.

FIG. 197B: Nucleotide sequence of intergenic region between AraC and GBS 80 (SEQ ID NO:273.

FIG. 197C: FACS analysis results for GBS 80 expression GBS strains having different length polyA tracts in the intergenic region between AraC and GBS 80.

FIG. 198: Table comparing the percent identity of surface proteins encoded by a serotype M6 (harbouring a GAS AI-1) adhesin island relative to other GAS serotypes harbouring an adhesin island.

FIG. 199: Table comparing the percent identity of surface proteins encoded by a serotype M1 (harbouring a GAS AI-2) adhesin island relative to other GAS serotypes harbouring an adhesin island.

FIG. 200: Table comparing the percent identity of surface proteins encoded by serotypes M3, M18, M5, and M49 (harbouring GAS AI-3) adhesin islands relative to other GAS serotypes harbouring an adhesin island.

FIG. 201: Table comparing the percent identity of surface proteins encoded by a serotype M12 (harbouring a GAS AI-1) adhesin island- relative to other GAS serotypes harbouring an adhesin island.

FIG. 202: GBS 80 recombinant protein does not bind to epithelial cells. Epithelial cells were incubated in the presence or absence of GBS80 protein and then a mouse a-GBS80 polyclonal antibody added. The cell were then stained with FITC-conjugated a-mouse IgG antibody. The violet area indicates cells treated with FITC-conjugated antibody alone. GBS80 binding, expressed as Dₑₘₑₐₙ channel values, was measured by FACScan cytometer as difference in fluorescence intensity between cell incubated with or without GBS80. The same protocol was used for GBS 104 protein binding to epithelial cells.

FIG. 203: Deletion of GBS 80 protein does not affect the ability of GBS to adhere and invade ME180 cervical epithelial cells. ME180 cervical carcinoma epithelial cells were infected with GBS 2603 wild type or 2603 D80 isogenic mutant. After 2 h infection, non-adherent bacteria were washed off and infection prolonged for further 2 h and 4 h. In invasion experiments, after each time point followed a 2 h antibiotic treatment. Cells were then lysed with 1% saponin and lysates plated on TSA plates.

FIG. 204: GBS 80 binds to extracellular matrix proteins. ELISA with purified ECM components and native GBS80 protein.

FIG. 205: Deletion of GBS 104 protein, but not GBS 80, reduces the capacity of GBS to invade J774 macrophage-like cells. J774 cells were infected with GBS COH1 wild type or COH1DGBS104/COH1DGBS80 isogenic mutants. After 1 h infection, non-adherent bacteria were washed off and intracellular bacteria recovered at 2 h, 4 h and 6 h post-antibiotic treatment. At each time point cells were lysed with 0.25% Triton X-100 and lysates plated on TSA plates.

FIG. 206: GBS 104 knockout mutant strains of bacteria translocate through an epithelial monolayer less efficiently that the isogenic wild type strain.

FIG. 207: GBS 80 knockout mutant strains of bacteria partially lose the ability to translocate through an epithelial monolayer. Epithelial cells monolayers were inoculated with each bacterium in the apical chamber of a transwell system for 2 h and then non-adherent bacteria washed off. Infection was prolonged for further 2 h and 4 h. Samples were taken from the media of the basolateral side and the number of colony forming units measured. Transepithelial electrical resistance measured prior and after infection gave comparable values, indicating the maintenance of the integrity of the monolayer.

FIG. 208: GBS adherence to HUVEC endothelial cells. HUVEC cells were infected with GBS COH1 wild type or COH1DGBS104/COH1DGBS80 isogenic mutants. After 1 h infection, non-adherent bacteria were washed off and cells lysed with 1% saponin and lysates plated on TSA plates.

FIG. 209: Strain growth rate of wildtype, GBS 80-deleted, or GBS 104 deleted COH1 GBS.

FIG. 210: Binding of recombinant GBS 104 protein to epithelial cells by FACS analysis.

FIG. 211: Deletion of GBS 104 protein in the GBS strain COH1 reduces the ability of GBS to adhere to ME180 cervical epithelial cells. ME180 cervical carcinoma epithelial cells were infected with GBS COH1 wild type or COH1DGBS104/COH1DGBS80 isogenic mutants. After 1 h infection, non-adherent bacteria were washed off and cells lysed with 1% saponin and lysates plated on TSA plates.

FIG. 212: COH1 strain GBS overexpressing GBS 80 protein has an impaired capacity to translocate through an epithelial monolayer.

FIG. 213: Scanning electron microscopy shows that overexpression of GBS 80 protein on COH1 strain GBS enhances the capacity of the COH1 bacteria to form microcolonies on epithelial cells.

FIG. 214: Confocal imaging shows that overexpression of GBS 80 proteins on COH1 strain GBS enhances the capacity of the COH1 bacteria to form microcolonies on epithelial cells.

FIG. 215: Detection of GBS 59 on the surface of GBS strain 515 by immuno-electron microscopy.

FIG. 216: Detection of GBS 67 on the surface of GBS strain 515 by immuno-electron microscopy.

FIG. 217: GBS 67 binds to fibronectin.

FIG. 218: Western blot analysis shows that deletion of both GBS AI-2 sortase genes abolishes assembly of the pilus.

FIG. 219: FACS analysis shows that deletion of both GBS AI-2 sortase genes abolishes assembly of the pilus.

FIG. 220A-C: Western blot analysis shows that GBS 59, GBS 67, and GBS 150 form high molecular weight complexes.

FIG. 221A-C: Western blot analysis shows that GBS 59 is required for polymer formation of GBS 67 and GBS 150.

FIG. 222: FACS analysis shows that GBS 59 is required for surface exposure of GBS 67.

FIG. 223: Summary Western blots for detection of GBS 59, GBS 67, or GBS 150 in GBS 515 and GBS 515 mutant strain.

FIG. 224: Description of GBS 59 allelic variants.

FIG. 225: GBS 59 is opsonic only against a strain of GBS expressing a homologous GBS 59.

FIGS. 226A and B: Results of FACS analysis for surface expression of GBS 59 using antibodies specific for different GBS 59 isoforms.

FIGS. 227A and B: Results of FACS analysis for surface expression of GBS 80, GBS 104, GBS 322, GBS 67, and GBS 59 on 41 various strains of GBS bacteria.

FIG. 228: Results of FACS analysis for surface expression of GBS 80, GBS 104, GBS 322, GBS 67, and GBS 59 on 41 strains of GBS bacteria obtained from the CDC.

FIG. 229: Expected immunogenicity coverage of different combinations of GBS 80, GBS 104, GBS 322, GBS 67, and GBS 59 across strains of GBS bacteria.

FIG. 230: GBS 59 opsonophagocytic activity is comparable to that of a mixture of GBS 80, GBS 104, GBS 322 and GBS 67.

FIG. 231A-C: Schematic presentation of example hybrid GBS AIs.

FIG. 232: Schematic presentation of an example hybrid GBS AI.

FIGS. 233A and B: Western blot and FACS analysis detect expression of GBS 80 and GBS 67 on the surface of L. lactis transformed with a hybrid GBS AI.

FIG. 234A-E Hybrid GBS AI cloning strategy.

FIG. 235: High magnification of S. pneumoniae strain TIGR4 pili double labeled with α-RrgB (5 nm) and α-RrgC (10 nm). Bar represents 100 nm.

FIG. 236: Immuno-gold labeling of the S. pneumoniae TIGR4 rrgA-srtD deletion mutant with no visible pili on the surface detectable by α-RrgB- and α-RrgC. Bar represents 200 nm.

FIG. 237: Variability in GBS 67 amino acid sequences between strains 2603 and H36B,

FIG. 238: Strain variability in GBS 67 amino acid sequences of allele I (2603).

FIG. 239: Strain variability in GBS 67 amino acid sequence of allele II (H36B).

FIG. 240: sequence identity dendrogram showing six GBS59 polypeptide allelic families.

FIG. 241. Immunogenicity of pilus subunits in humans. FIG. 241A, FACS analysis of ability of human sera to recognize whole S. pneumoniae TIGR4 cells. P=sera from patients with diagnosed pneumococcal diseases; H=serum from a healthy donor. FIG. 241B, Western blot detection of S. pneumoniae TIGR4 mutanolysin preparation by human sera. Representative results obtained with three sera are shown. The typical ladder constituted by polymers of pilus subunits, shown by silver staining (SS), is recognized by the patient's sera (P) but not by the healthy donor control (H). Electrophoretic migrations of relevant molecular mass markers are indicated on the left. FIG. 241C, ELISA quantification of specific IgG against recombinant RrgA, RrgB or RrgC in human sera from patients (P) with diagnosed pneumococcal diseases (N=9) or from a healthy donor (H) as indicated. Sera were diluted 1:500. For the sera from the patients: columns=mean of the 9 sera; bars=standard deviation.

FIG. 242. Immunogenicity of pilus subunits in mice. ELISA quantification of specific IgG titers against recombinant RrgA, RrgB or RrgC in sera of mice immunized as indicated. N=8 for each group with the exception of control group in which N=16. Specific IgG were undetectable in control group (adjuvant plus saline). Columns represent the mean of the group, with the exception of the S. pneumoniae TIGR4 vaccination group, in which sera were pooled. A+B+C=combination of RrgA+B+C ; bars=standard deviation.

FIG. 243. Protective efficacy of pilus subunits in mice. Protective efficacy against S. pneumoniae TIGR4 or 6B challenge of active vaccination with either recombinant pilus antigens or heat-inactivated S. pneumoniae TIGR4 with Freund's adjuvant or Al(OH)₃ as indicated, or passive transfer of antisera raised against the same antigens with Freund's adjuvant. N=8 for each group with the exception of control groups in the Freund's adjuvant and in the passive immunization panels, in which N=16. FIG. 243A, Bacteremia at 24 h (for S. pneumoniae TIGR4 challenge) or 5 h (for 6B challenge) post-challenge. Circles=values of CFU per ml of blood of single animals; horizontal bars=geometric mean of each group; dashed line=detection limit (i.e. no CFU were detected in blood samples below dashed line). FIG. 243B, Mortality course. Diamonds=survival days of single animals, horizontal bars=median of survival days of each group; dashed line=endpoint of observation (i.e. animals above the dashed line survived at the endpoint). ctrl=mice receiving only the corresponding adjuvant plus saline; A+B+C=combination of RrgA+B+C; *=P<0.05 and **=P<0.01, in comparison with the corresponding control group.

FIG. 244. Schematic of M2 (Adhesin-Island 5) andM4 (Adhesin-Island 6).

FIG. 245. Schematic of GAS M2 AI-5.

FIG. 246. Schematic of GAS M4 AI-6.

FIG. 247A-D. Schematic of sequence identity of AI proteins (see SEQ ID NOS:318-466).

FIG. 248 Immunoblots on cell-wall fractions and immuno-electron microscopy images of GAS strain SF370 wild type, Δspy0128, Δspy0129, Δspy0128/pAM::128, Δspy0129/pAM::129, and L. lactis strains MG1363/pAM, MG1363/pAM::pilM1. For immunoblots, sera against Cpa (GAS15), Backbone (GAS16/spy0128) and Spy0130 (GAS18) were used on cell-wall fractions of each strain. For immuno-electron microscopy bacteria were labeled with serum against Backbone. As a negative control pre-immune sera were used. Bars: 200 nm.

FIG. 249. FIGS. 249A-E, confocal microscopy images of GAS SF370 wild type, Δspy0128, Δspy0129, Δspy0128/pAM::128 and Δspy0129/pAM::129 grown to late exponential growth phase on polylysine-coated coverslips and stained with anti-GAS (blue) and anti-spy0128 (red) sera. FIGS. 249F-G, light microscopy analysis of L. lactis MG1363 strains transformed with pAM vector alone or with pAM::pilM1.

FIG. 250. Confocal microscopy images of bacterial aggregation on human pharynx cell line Detroit-562. Cell monolayers were incubated with bacteria grown to OD 0.4 at 37° C. in a 5% CO2 atmosphere; after 15 minutes of incubation wells were extensively washed 3 times with PBS to remove the unattached bacteria, and infection was let continue to 30, 60 and 120 minutes, then wells were washed again and stained with anti-phalloidin (blue) for eukaryotic cell staining and a polyclonal anti-GAS serum (green) for bacteria staining Panels A-D: SF370 wild type; panels E-H Δspy0128; panels I-L Δpy0129.

FIG. 251. FIGS. 251A-B, adherence assay with SF370 wild type, Δspy128 and Δspy0129 on Detroit-562 pharynx cell line. Confluent cell monolayers were infected with bacteria (MOI 100:1) for 5, 15, 30 and 120 minutes. The percentage of adherent bacteria was calculated as follows: (n. bacteria recovered after infection/n. inoculated bacteria)×100 and reported as real percentage (A) or as normalized percentage considering the wild type as 100% (B). FIG. 251C: Adherence assay with L. lactis transformed with pAM401 vector alone and pAM401 containing pilus region of M1 SF370 on Detroit-562 pharynx cell line. A MOI 10:1 was used to infect cells for 15 and 120 min. Each experiment was performed in triplicate and repeated three times. Means and standard deviations of three experiments are shown.

FIG. 252. Bio-film assay with SF370 wt, Δspy128, Δspy0129, Δspy128/pAM::128, Δspy0129/pAM::129 strains. Bacteria were incubated in C-medium at room temperature on 24-multi well plates and at the indicated time points supernatant were removed and adherent bacteria were stained with crystal violet 0.2%. Photograph (A) and quantification by measurement of OD at 540 nm (B) of bacterial adhesion after 24 h incubation. Bacterial growth in the same conditions for 24 h was also checked as a control. Experiments were performed in triplicate and repeated at least three times. Means and standard deviations of one representative experiment are shown.

FIG. 253. X-Z and X-Y panels and three dimensional views of 72 h bio-films formed by SF370 wild type (A), Δspy0128 (B), Δspy128/pAM::128 (C), Δspy0129 (D), Δsp\tilde{y}012\tilde{9}/pAM::129 (E). Bacteria were grown in C-medium for 72 hours at room temperature on polylysine-coated coverslips placed at the bottom of 50 ml tubes. Medium was changed every 24 hours. Coverslips were then recovered, fixed and stained with anti-GAS (blue) and anti-spy128 (red) sera, and with FITC conjugated-ConA (green). Thickness of bio-films was also measured in different points of each field, and the average of at least 6 measures with standard deviations are reported in the table.

FIG. 254. Graphs showing that passive transfer of antisera to S. pneumoniae TIGR4 native pilus protects against S. pneumoniae TIGR4 challenge.

FIG. 255. Correlation between Pilus Islands distribution and GBS capsular serotype. The different colors represent the pilus islands combination found in the clinical isolates, as shown in the legend. The number of isolates containing the different types of pilus islands is indicated inside each column.

FIG. 256. Schematic representation of sequence variability of pilus-coding genes among GBS clinical isolates. (A), (B) and (C) indicate gene conservation in isolates containing PI-1, PI-2a and PI-2b, respectively. The total number of strains containing the same PI allele and their serotype distribution are boxed at the right side of each allele. Grey arrows represent the sortases genes present in each island. Sequences with 100% identity are shown in the same color, while variants showing less than 90% sequence identity are indicated in different colors. Single mutations are represented with vertical bars and the number above each bar indicates the position/substitution of the mutated residue. (D) Phylogenetic trees inferred from the protein alignments by the neighbour-joining-distance-based method of variants of BP-2a and of AP1-2a. Numbers at the nodes indicate bootstrap values.

FIG. 257. Correlation between the presence of pilus islands (PCR positive, red columns) and surface exposure of pili structural components measured by flow cytometry as the difference in fluorescence between cells stained with immune sera versus pre-immune sera. Numbers inside each column represent the number of strains that belong to each group, whereas the numbers shown at the top of columns indicate the average values of fold increase in fluorescence and the corresponding standard deviation. Blue color columns represent the number of strains showing a greater than 2-fold increase in fluorescence. Yellow columns indicate the number of strains showing a greater than 5-fold increase in fluorescence.

### DETAILED DESCRIPTION OF THE INVENTION

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, Mack Publishing Company, Easton, Pa., 19th Edition (1995); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B. N. Raven Press, New York, N.Y.

All publications, patents and patent applications cited herein, are hereby incorporated by reference in their entireties.

As used herein, an “Adhesin Island” or “AI” refers to a series of open reading frames within a bacterial genome, such as the genome for Group A or Group B Streptococcus or other gram positive bacteria, that encodes for a collection of surface proteins and sortases. An Adhesin Island may encode for amino acid sequences comprising at least one surface protein. The Adhesin Island may encode at least one surface protein. Alternatively, an Adhesin Island may encode for at least two surface proteins and at least one sortase. Preferably, an Adhesin Island encodes for at least three surface proteins and at least two sortases. One or more of the surface proteins may include an LPXTG motif (e.g., SEQ ID NO:122) or other sortase substrate motif. One or more AI surface proteins may participate in the formation of a pilus structure on the surface of the gram positive bacteria.

Adhesin Islands of the invention preferably include a divergently transcribed transcriptional regulator (i.e., the transcriptional regulator is located near or adjacent to the AI protein open reading frames, but it transcribed in the opposite direction). The transcriptional regulator may regulate the expression of the AI operon.

GBS Adhesin Island 1

As discussed above, Applicants have identified a new adhesin island, “Adhesin Island 1,” “AI-1,” or “GBS AI-1,” within the genomes of several Group B Streptococcus serotypes and isolates. AI-1 comprises a series of approximately five open reading frames encoding for a collection of amino acid sequences comprising surface proteins and sortases (“AI-1 proteins”). Specifically, AI-1 includes open reading frames encoding for two or more (i.e., 2, 3, 4 or 5) of GBS 80, GBS 104, GBS 52, SAG0647 and SAG0648. One or more of the AI-1 open reading frame polynucleotide sequences may be replaced by a polynucleotide sequence coding for a fragment of the replaced ORF. Alternatively, one or more of the AI-1 open reading frames may be replaced by a sequence having sequence homology to the replaced ORF.

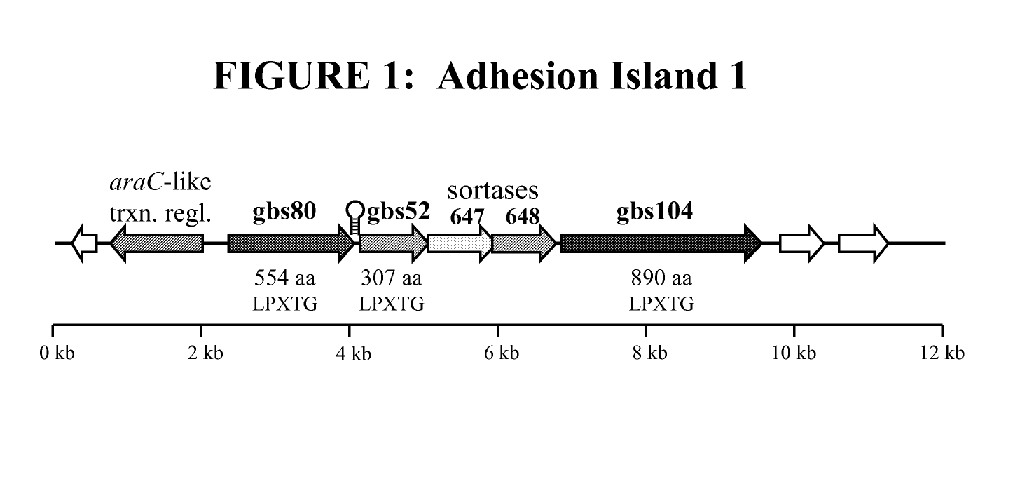

A schematic of AI-1 is presented in FIG. 1. AI-1 typically resides on an approximately 16.1 kb transposon-like element frequently inserted into the open reading frame for trmA. One or more of the AI-1 surface protein sequences typically include an LPXTG motif (e.g., SEQ ID NO:122) motif or other sortase substrate motif. The AI surface proteins of the invention may affect the ability of the GBS bacteria to adhere to and invade epithelial cells. AI surface proteins may also affect the ability of GBS to translocate through an epithelial cell layer. Preferably, one or more AI surface proteins are capable of binding to or otherwise associating with an epithelial cell surface. AI surface proteins may also be able to bind to or associate with fibrinogen, fibronectin, or collagen.

The AI-1 sortase proteins are predicted to be involved in the secretion and anchoring of the LPXTG containing surface proteins. AI-1 may encode for at least one surface protein. Alternatively, AI-1 may encode for at least two surface exposed proteins and at least one sortase. Preferably, AI-1 encodes for at least three surface exposed proteins and at least two sortases. The AI-1 protein preferably includes GBS 80 or a fragment thereof or a sequence having sequence identity thereto.

As used herein, an LPXTG motif represents an amino acid sequence comprising at least five amino acid residues. Preferably, the motif includes a leucine (L) in the first amino acid position, a proline (P) in the second amino acid position, a threonine (T) in the fourth amino acid position and a glycine (G) in the fifth amino acid position. The third position, represented by X, may be occupied by any amino acid residue. Preferably, the X is occupied by lysine (K), Glutamate (E), Asparagine (N), Glutamine (Q) or Alanine (A). Preferably, the X position is occupied by lysine (K). In some embodiments, one of the assigned LPXTG amino acid positions is replaced with another amino acid. Preferably, such replacements comprise conservative amino acid replacements, meaning that the replaced amino acid residue has similar physiological properties to the removed amino acid residue. Genetically encoded amino acids may be divided into four families based on physiological properties: (1) acidic (aspartate and glutamate), (2) basic (lysine, arginine, histidine), (3) non-polar (alanine, valine, leucine, isoleucine, proline, phenylalanine, methionine, tryptophan) and (4) uncharged polar (glycine, asparagines, glutamine, cysteine, serine, threonine, and tyrosine). Phenylalanine, tryptophan and tyrosine are sometimes classified jointly as aromatic amino acids. For example, it is reasonably predictable that an isolated replacement of a leucine with an isoleucine or valine, an aspartate with a glutamate, a threonine with a serine, or a similar conservative replacement of an amino acid with a structurally related amino acid will not have a major effect on the biological activity.

The first amino acid position of the LPXTG motif may be replaced with another amino acid residue. Preferably, the first amino acid residue (leucine) is replaced with an alanine (A), valine (V), isoleucine (I), proline (P), phenylalanine (F), methionine (M), glutamic acid (E), glutamine (Q), or tryptophan (Y) residue. In one preferred embodiment, the first amino acid residue is replaced with an isoleucine (I).

The second amino acid residue of the LPXTG motif may be replaced with another amino acid residue. Preferably, the second amino acid residue praline (P) is replaced with a valine (V) residue.

The fourth amino acid residue of the LPXTG motif may be replaced with another amino acid residue. Preferably, the fourth amino acid residue (threonine) is replaced with a serine (S) or an alanine (A).

In general, an LPXTG motif may be represented by the amino acid sequence XXXXG, in which X at amino acid position 1 is an L, a V, an E, an I, an F, or a Q; X at amino acid position 2 is a P if X at amino acid position 1 is an L, an I, or an F; X at amino acid position 2 is a V if X at amino acid position 1 is a E or a Q; X at amino acid position 2 is a V or a P if X at amino acid position 1 is a V; X at amino acid position 3 is any amino acid residue; X at amino acid position 4 is a T if X at amino acid position 1 is a V, E, I, F, or Q; and X at amino acid position 4 is a T, S, or A if X at amino acid position 1 is an L.

Generally, the LPXTG motif of a GBS AI protein may be represented by the amino acid sequence XPXTG, in which X at amino acid position 1 is L, I, or F, and X at amino acid position 3 is any amino acid residue. Specific examples of LPXTG motifs in GBS AI proteins may include LPXTG (SEQ ID NO:122) or IPXTG (SEQ ID NO:133).

As discussed further below, the threonine in the fourth amino acid position of the LPXTG motif may be involved in the formation of a bond between the LPXTG containing protein and a cell wall precursor. Accordingly, in preferred LPXTG motifs, the threonine in the fourth amino acid position is not replaced with another amino acid or, if the threonine is replaced, the replacement amino acid is preferably a conservative amino acid replacement, such as serine.

Instead of an LPXTG motif, the AI surface proteins of the invention may contain alternative sortase substrate motifs such as NPQTN (SEQ ID NO:142), NPKTN (SEQ ID NO:168), NPQTG (SEQ ID NO:169), NPKTG (SEQ ID NO:170), XPXTGG (SEQ ID NO:143), LPXTAX (SEQ ID NO:144), or LAXTGX (SEQ ID NO:145). (Similar conservative amino acid substitutions can also be made to these membrane motifs).

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

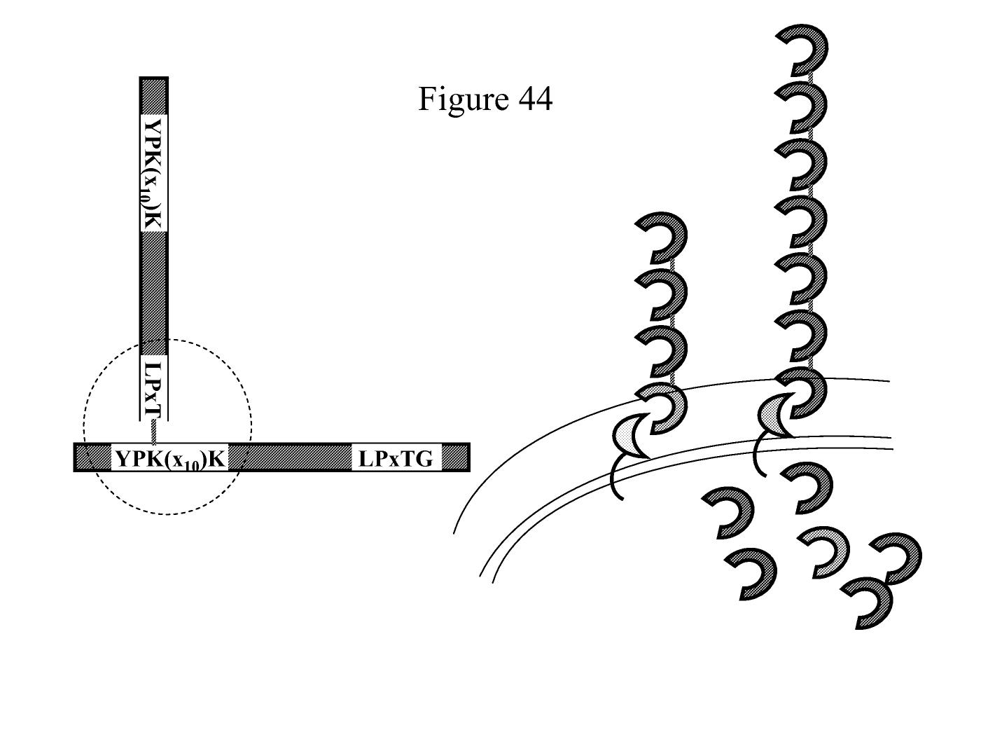

The AI surface proteins may be polymerized into pili by sortase-catalysed transpeptidation. (See FIG. 44.) Cleavage of AI surface proteins by sortase between the threonine and glycine residues of an LPXTG motif yields a thioester-linked acyl intermediate of sortase. Many AI surface proteins include a pilin motif amino acid sequence which interacts with the sortase and LPXTG amino acid sequence. The first lysine residue in a pilin motif can serve as an amino group acceptor of the cleaved LPXTG motif and thereby provide a covalent linkage between AI subunits to form pili. For example, the pilin motif can make a nucleophilic attack on the acyl enzyme providing a covalent linkage between AI subunits to form pili and regenerate the sortase enzyme. Examples of pilin motifs may include ((YPKN(X₁₀)K; SEQ ID NO:146), (YPKN(X₉)K; SEQ ID NO:147), (YPK(X₇)K; SEQ ID NO:148), (YPK(X₁₁)K; SEQ ID NO:149), or (PKN(X₉)K; SEQ ID NO:150)). Preferably, the AI surface proteins of the invention include a pilin motif amino acid sequence.

Typically, AI surface proteins of the invention will contain an N-terminal leader or secretion signal to facilitate translocation of the surface protein across the bacterial membrane.

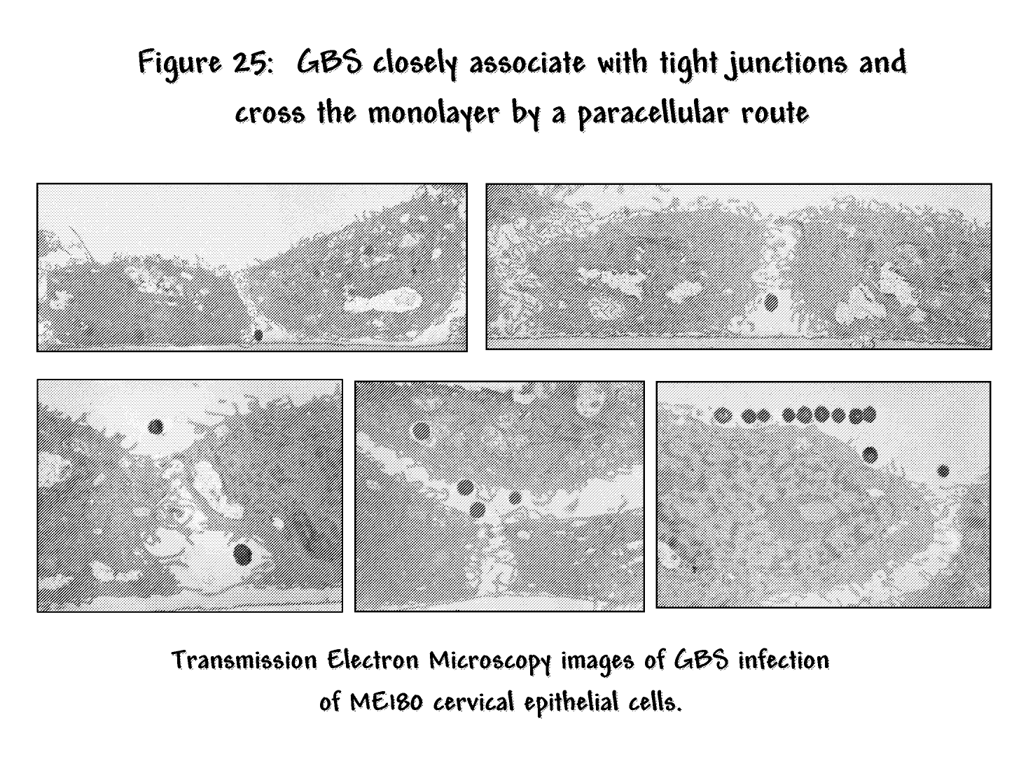

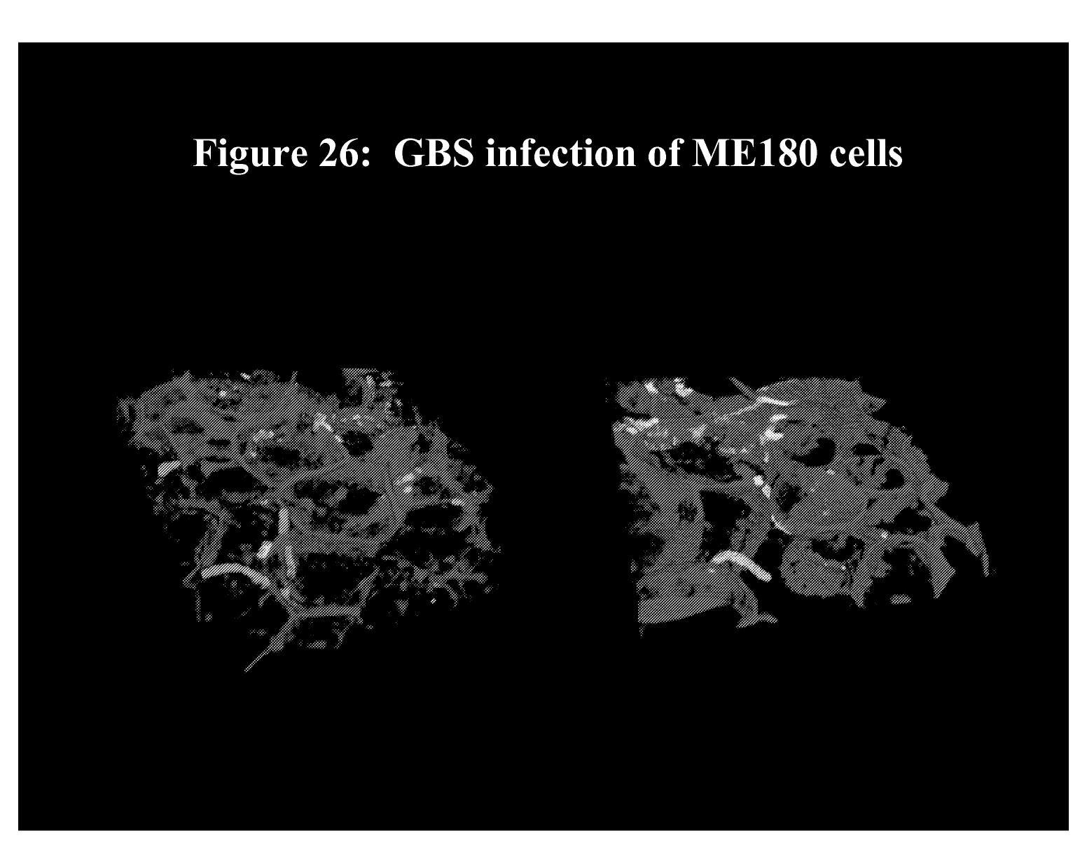

Group B Streptococci are known to colonize the urinary tract, the lower gastrointestinal tract and the upper respiratory tract in humans. Electron micrograph images of GBS infection of a cervical epithelial cell line (ME180) are presented in FIG. 25. As shown in these images, the bacteria closely associate with tight junctions between the cells and appear to cross the monolayer by a paracellular route. Similar paracellular invasion of ME180 cells is also shown in the contrast images in FIG. 26. The AI surface proteins of the invention may effect the ability of the GBS bacteria to adhere to and invade epithelial cells. AI surface proteins may also affect the ability of GBS to translocate through an epithelial cell layer. Preferably, one or more AI surface proteins are capable of binding to or otherwise associating with an epithelial cell surface.

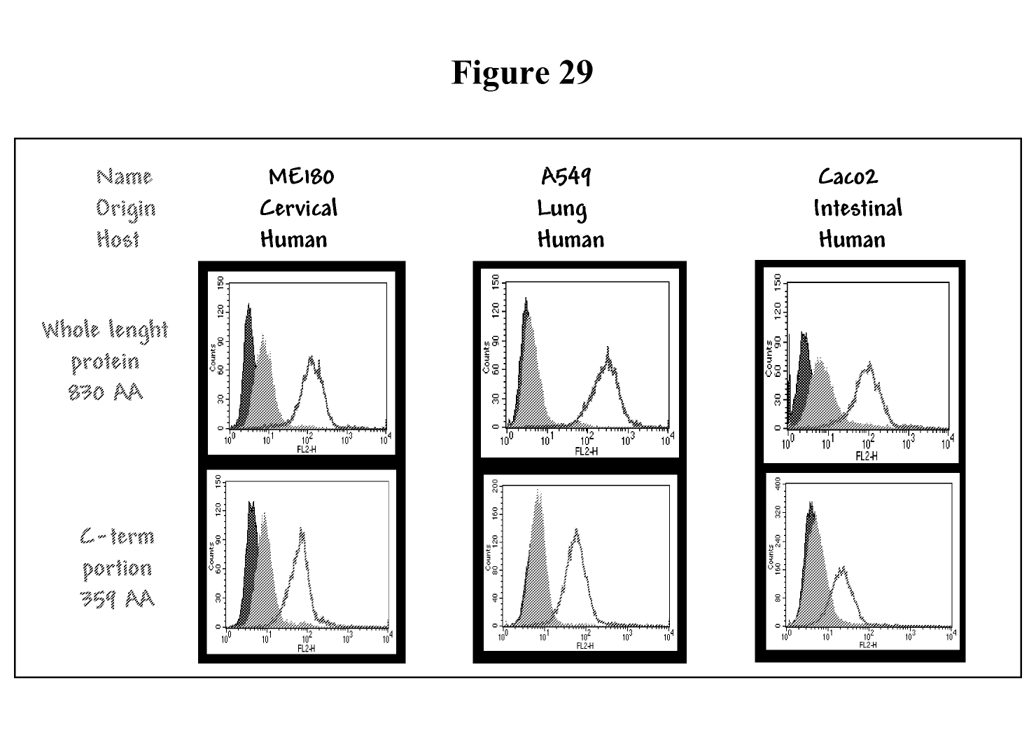

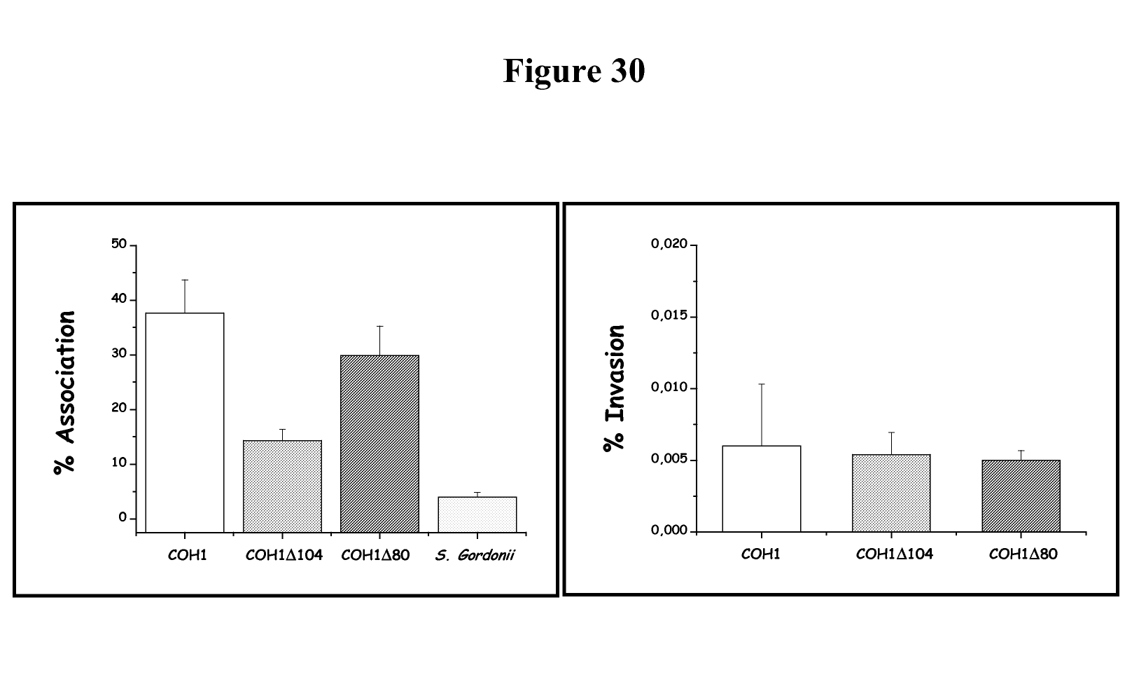

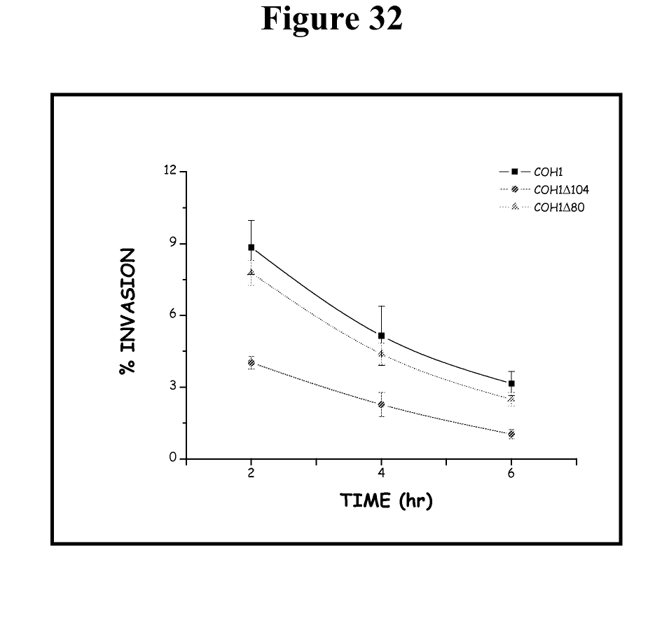

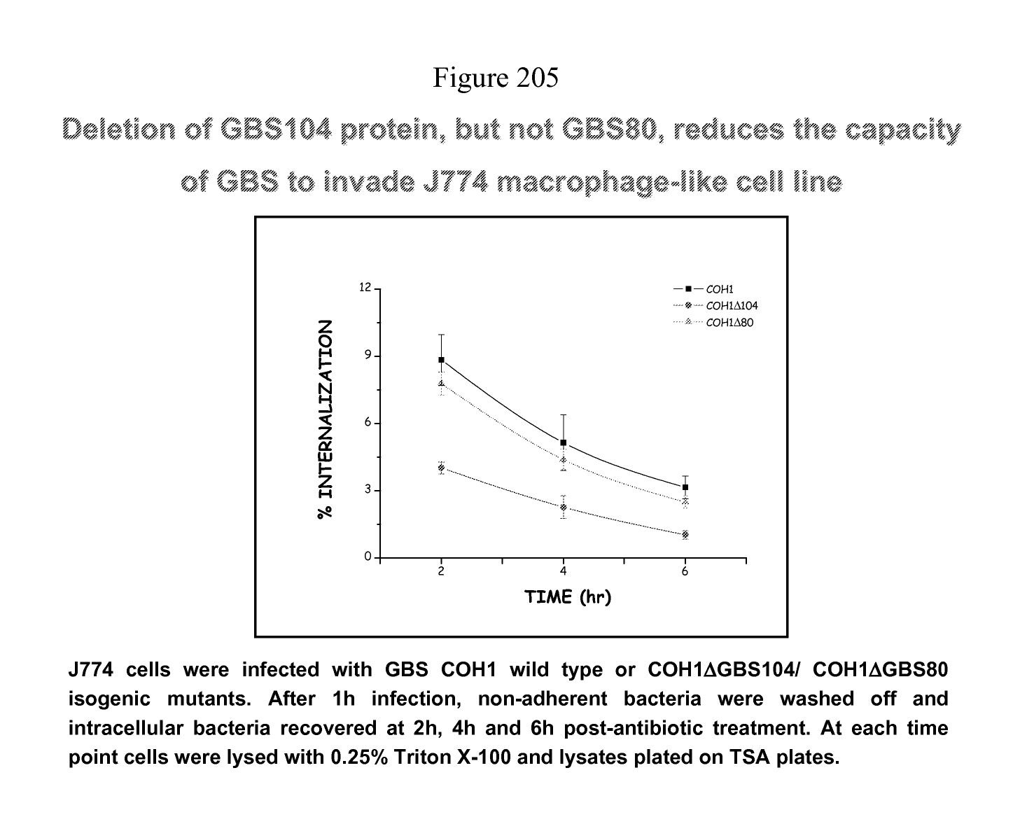

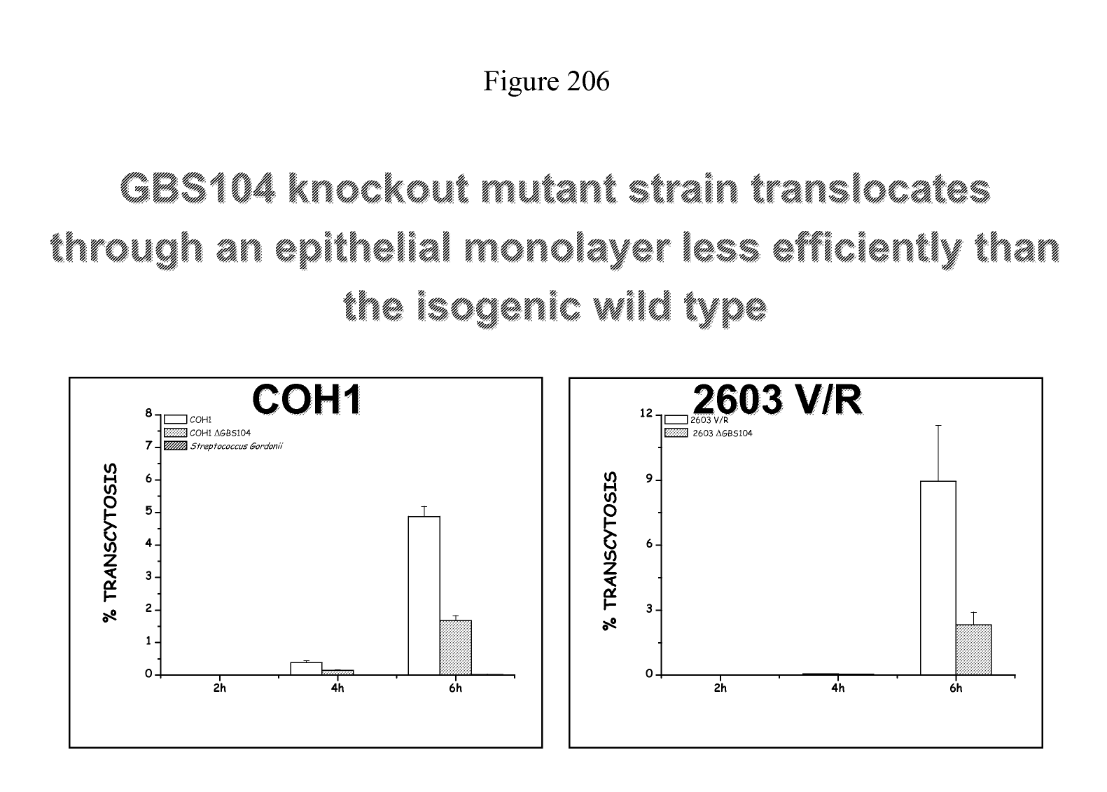

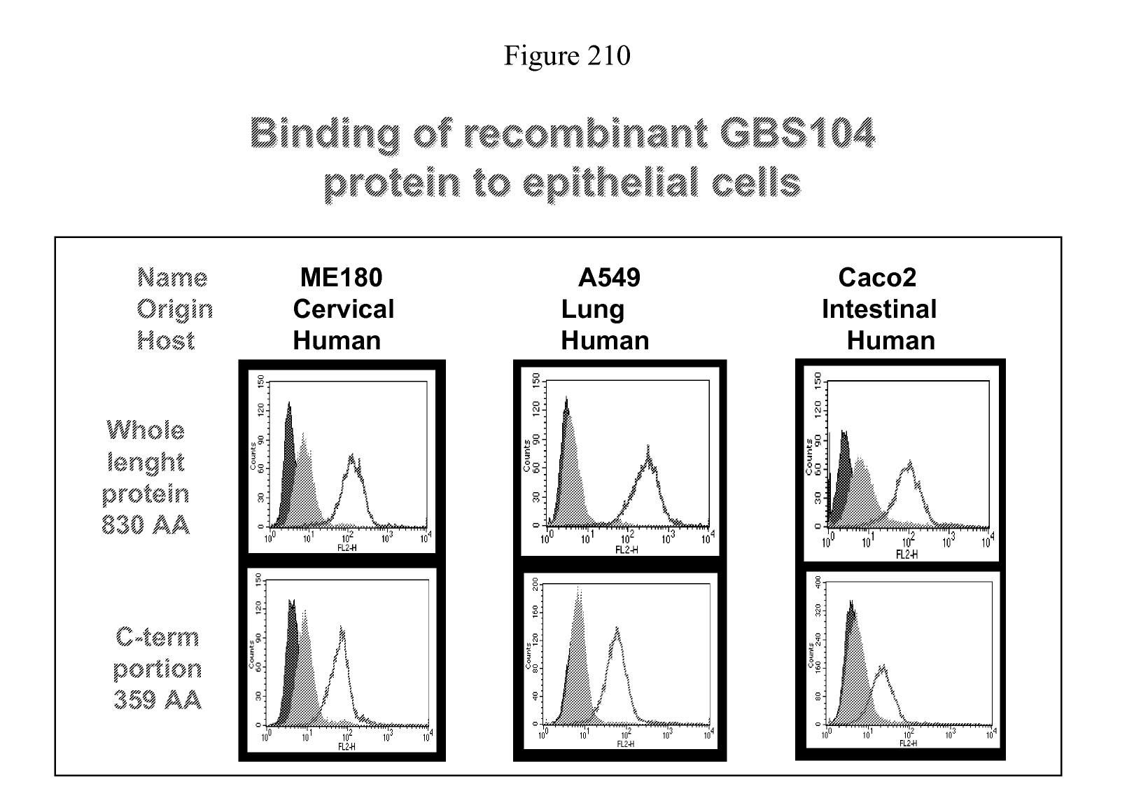

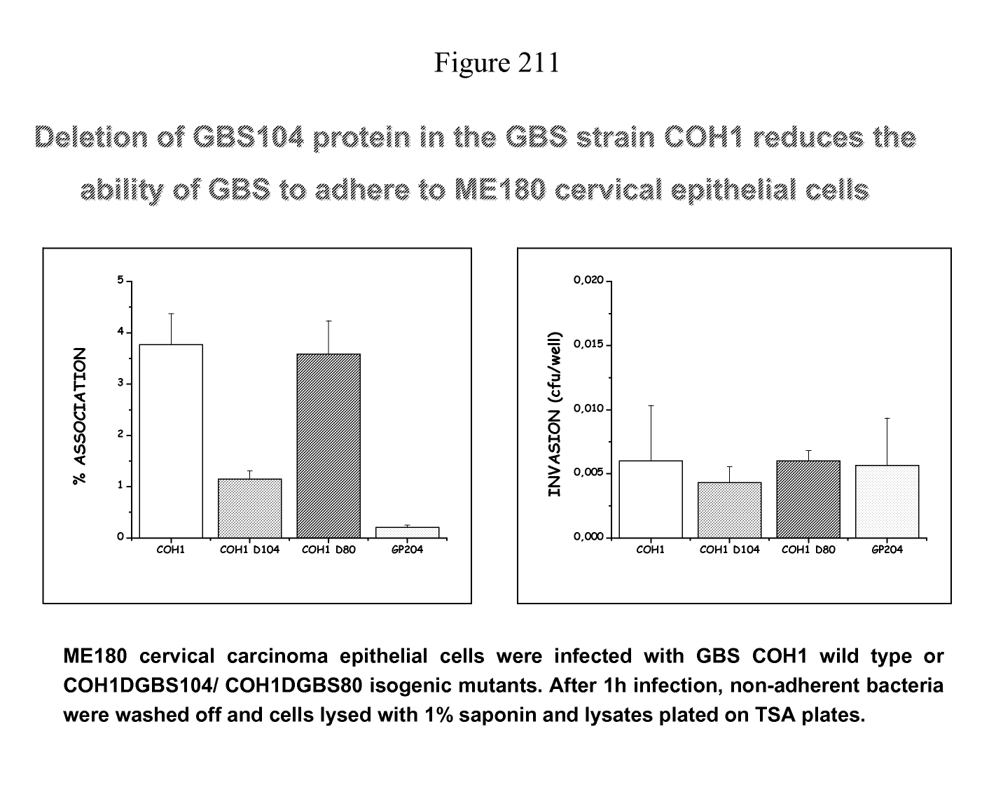

Applicants have discovered that AI-1 surface protein GBS 104 can bind epithelial cells such as ME180 human cervical cells, A549 human lung cells and Caco2 human intestinal cells (See FIGS. 29 and 210). Further, deletion of the GBS 104 sequence in a GBS strain reduces the capacity of GBS to adhere to ME180 cervical epithelial cells. (See FIGS. 30 and 211). Deletion of GBS 104 also reduces the capacity of GBS to invade J774 macrophage-like cells. (See FIGS. 32 and 205). Deletion of GBS 104 also causes GBS to translocate through epithelial monolayers less efficiently. See FIG. 206. GBS 104 protein therefore appears to bind to ME180 epithelial cells and to have a role in adhesion to epithelial cells and macrophage cell lines.

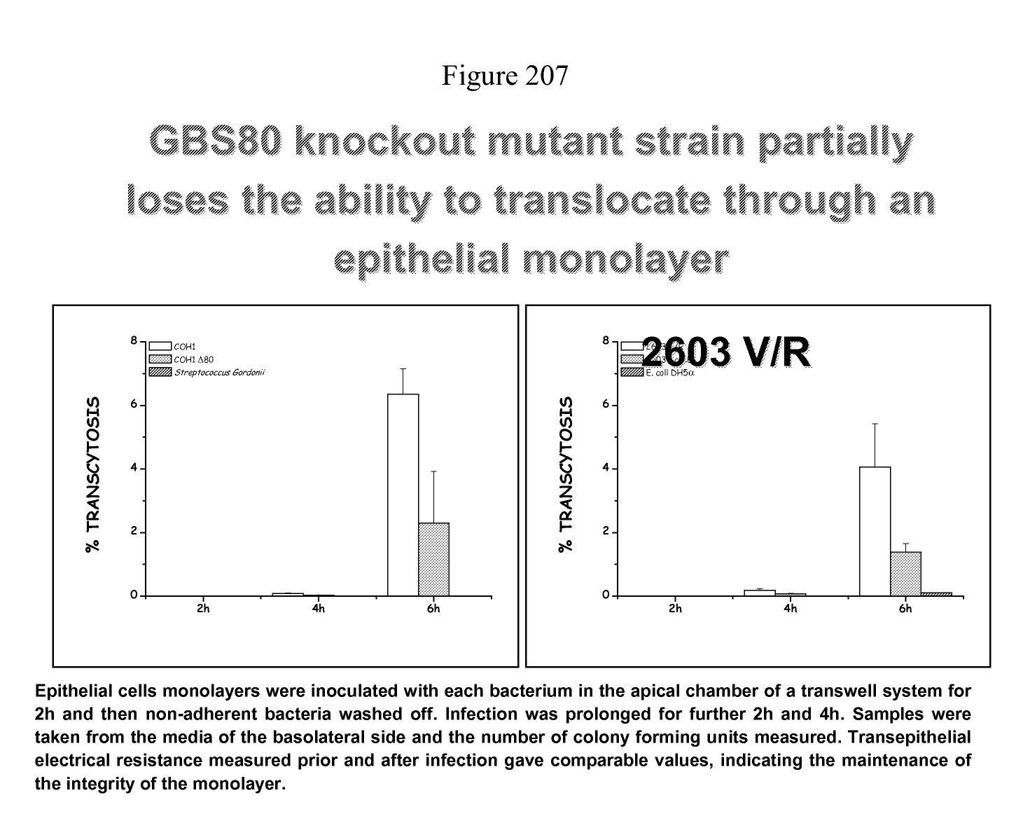

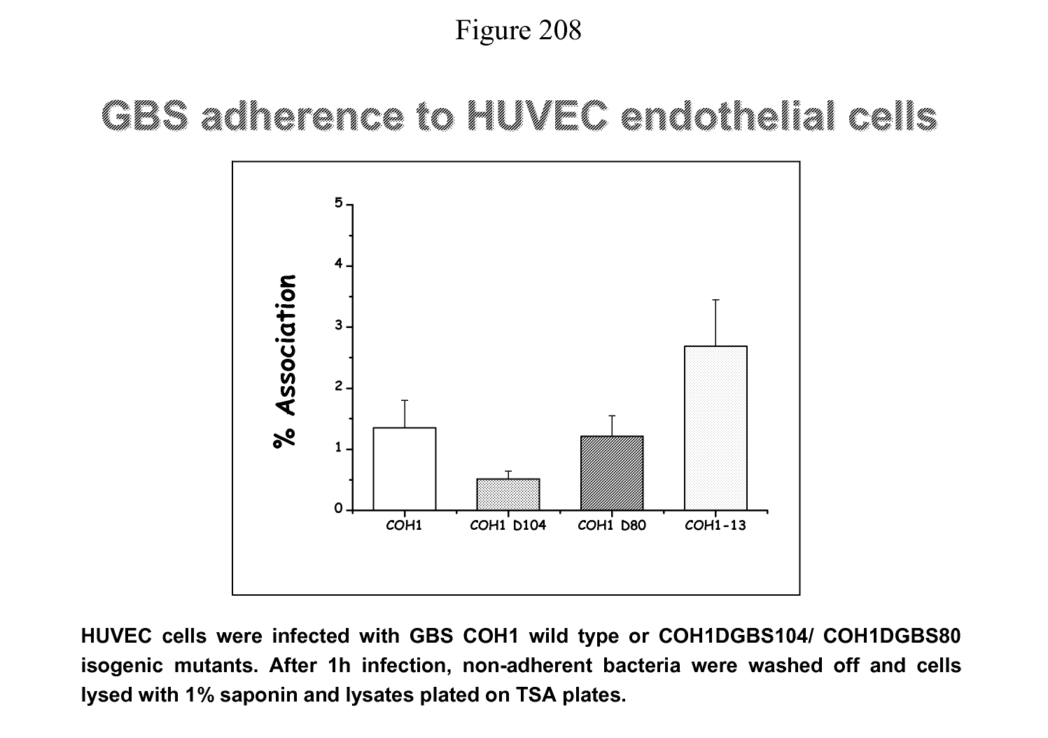

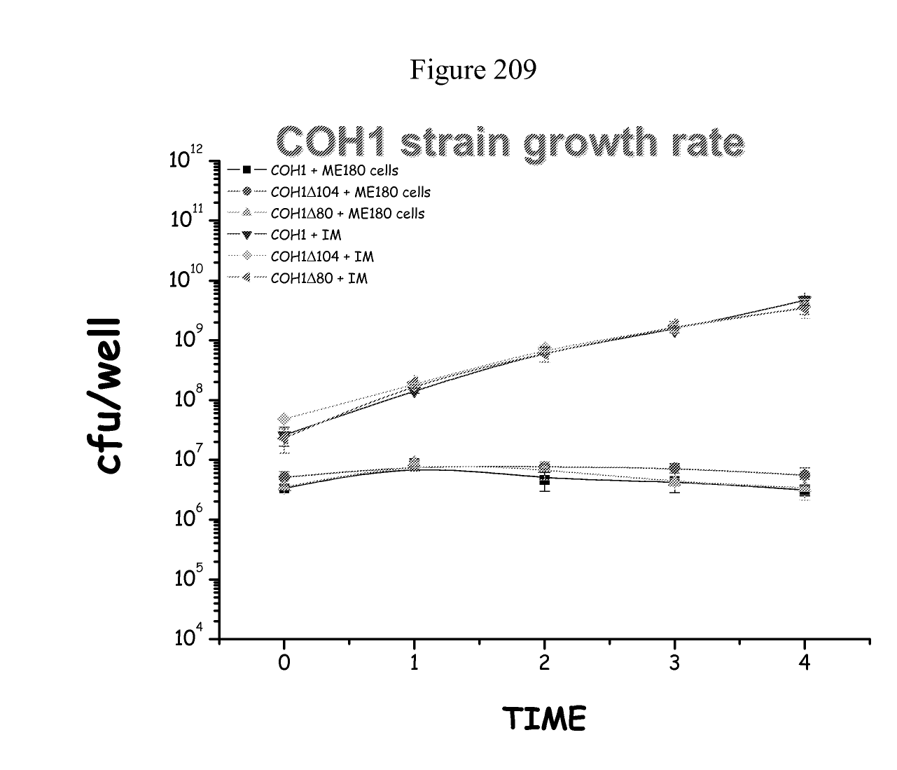

Similar to the GBS bacteria that are deletion mutants for GBS 104, GBS 80 knockout mutant strains also partially lose the ability to translocate through an epithelial monolayer. See FIG. 207. Deletion of either GBS 80 or GBS 104 in COH1 cells diminishes adherence to HUVEC endothelial cells. See FIG. 208. Deletion of GBS 80 or GBS 104 in COH1 does not, however, affect growth of COH1 either with ME180 cells or in incubation medium (IM). See FIG. 209. Both GBS 80 and GBS 104, therefore, appear to be involved in translocation of GBS through epithelial cells.

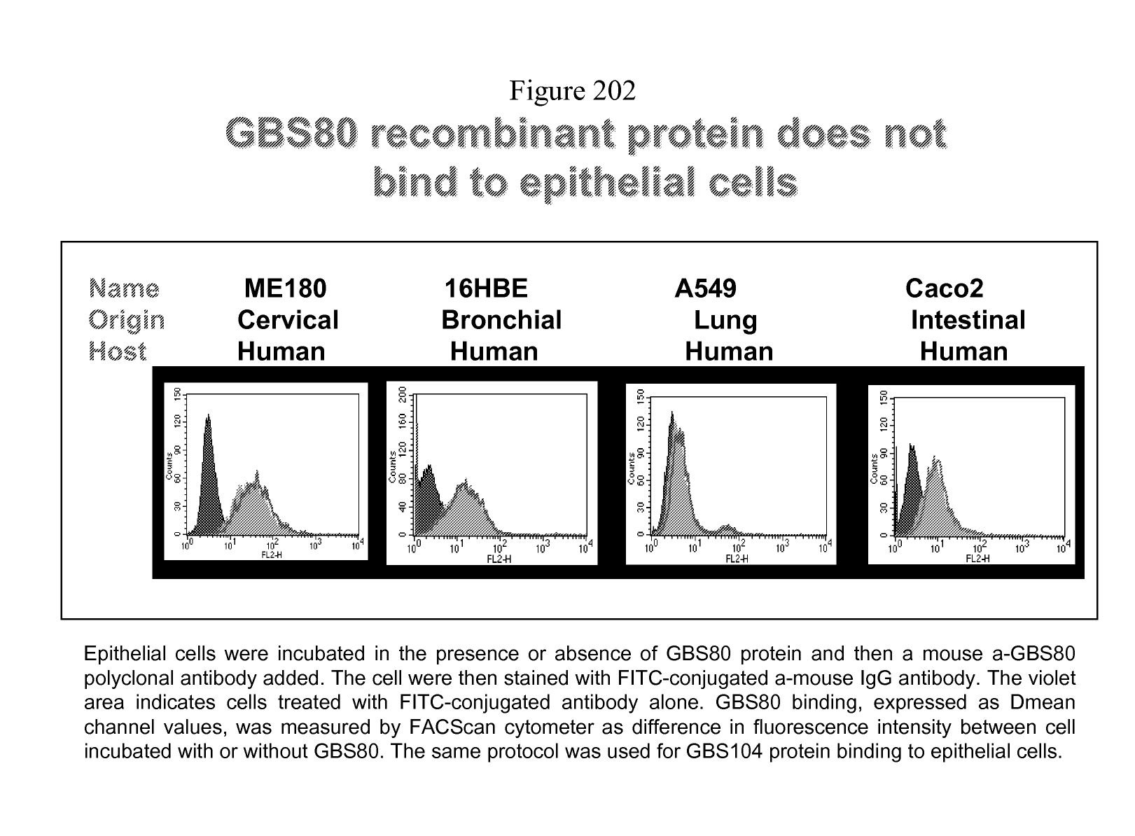

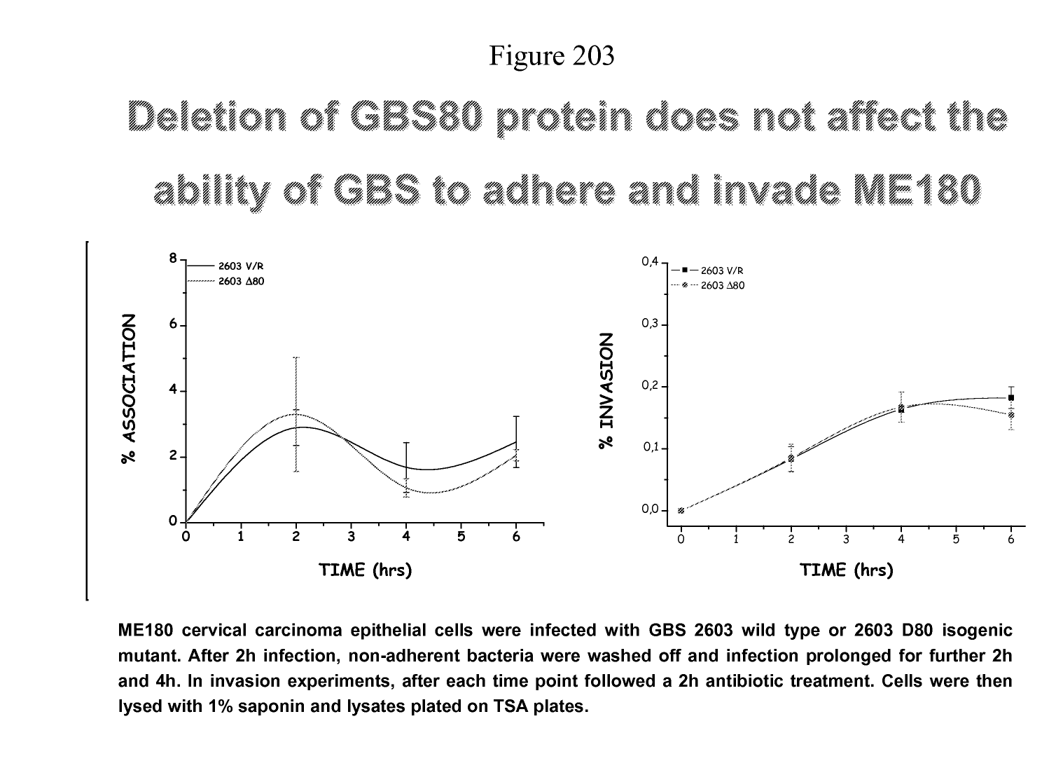

GBS 80 does not appear to bind to epithelial cells. Incubation of epithelial cells in the presence of GBS 80 protein followed by FACS analysis using an anti-GBS 80 polyclonal antibody did not detect GBS 80 binding to the epithelial cells. See FIG. 202. Furthermore, deletion of GBS 80 protein does not affect the ability of GBS to adhere and invade ME180 cervical epithelial cells. See FIG. 203

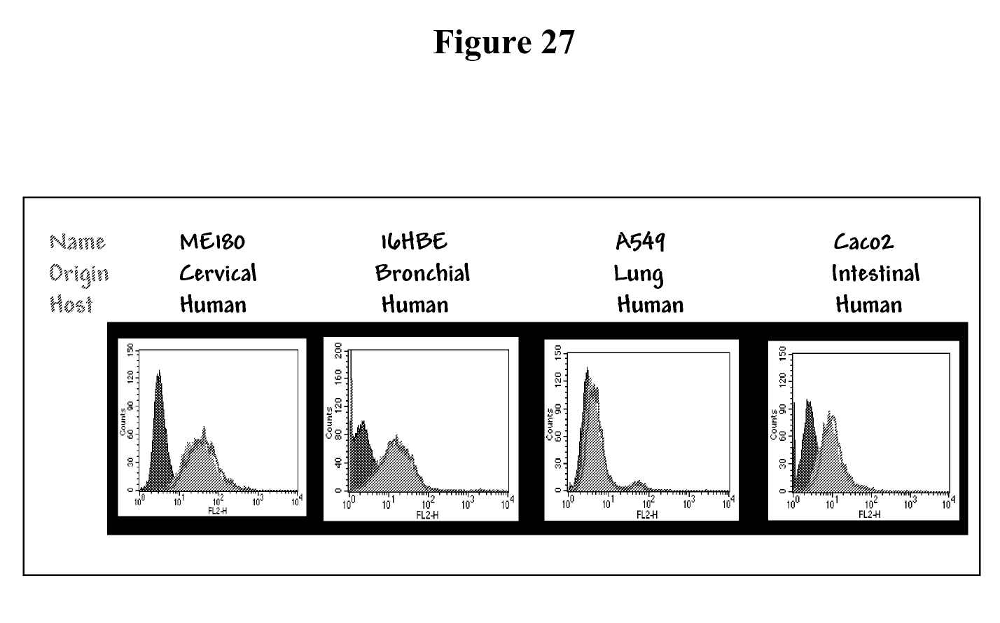

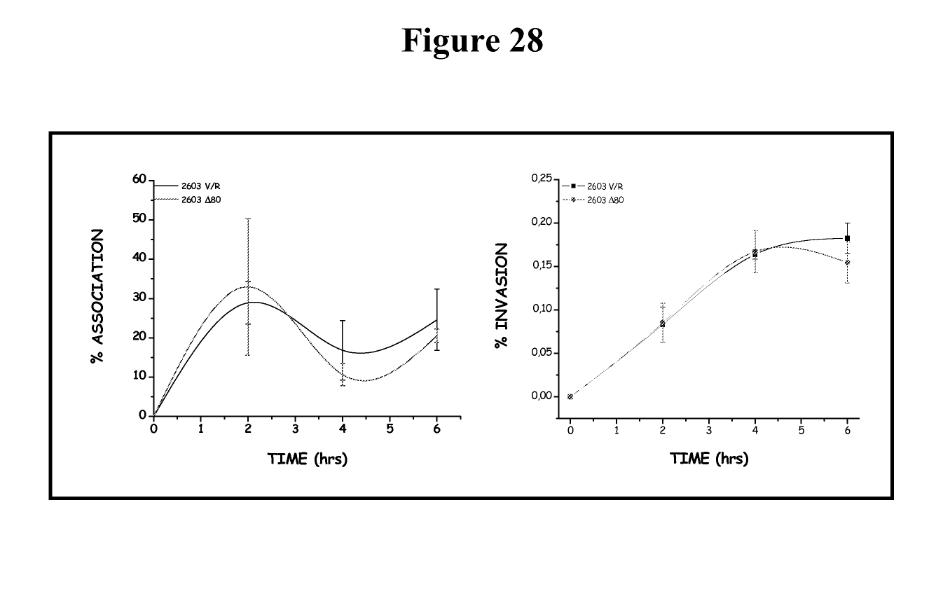

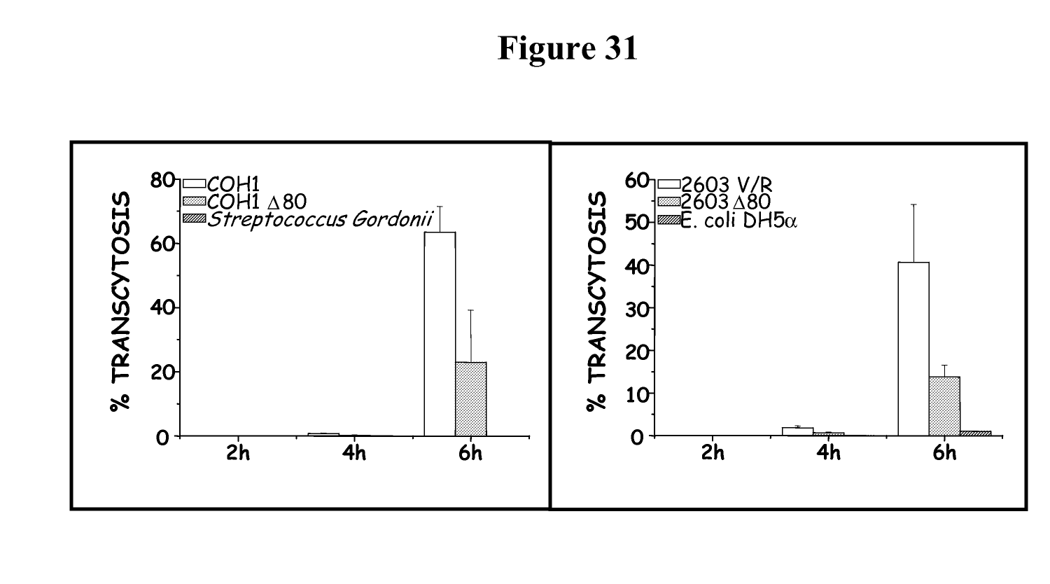

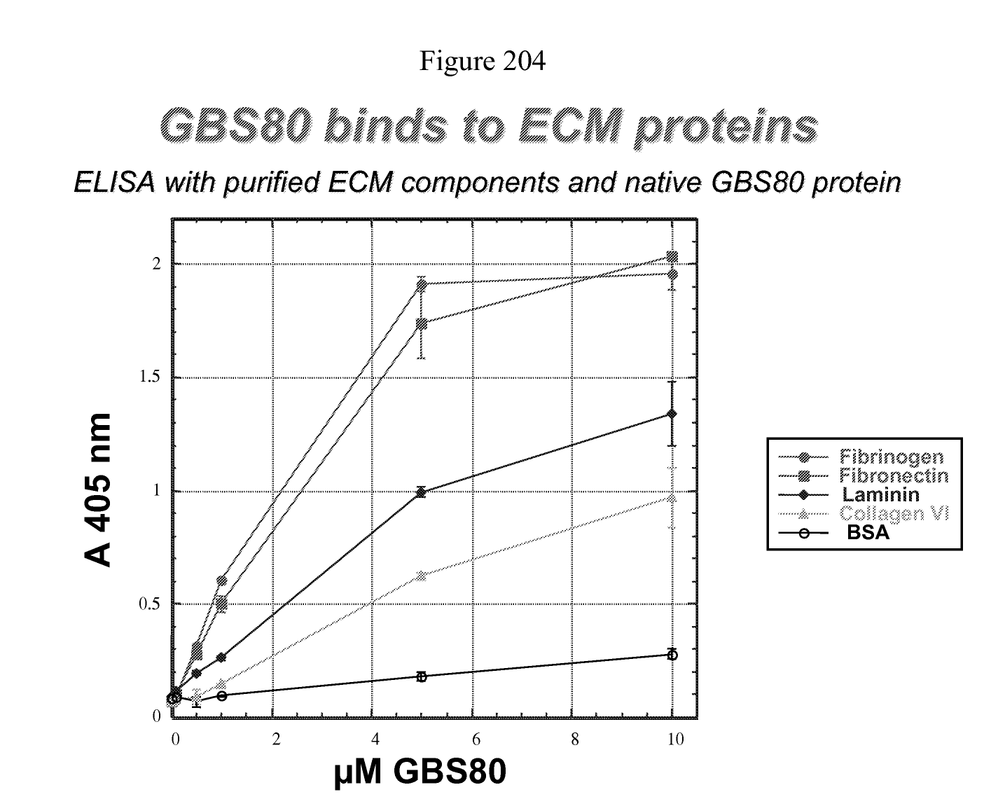

Preferably, one or more of the surface proteins may bind to one or more extracellular matrix (ECM) binding proteins, such as fibrinogen, fibronectin, or collagen. As shown in FIGS. 5 and 204, and Example 1, GBS 80, one of the AI-1 surface proteins, can bind to the extracellular matrix binding proteins fibronectin and fibrinogen. While GBS 80 protein apparently does not bind to certain epithelial cells or affect the capacity of a GBS bacteria to adhere to or invade cervical epithelial cells (See FIGS. 27 and 28), removal of GBS 80 from a wild type strain decreases the ability of that strain to translocate through an epithelial cell layer (see FIG. 31).

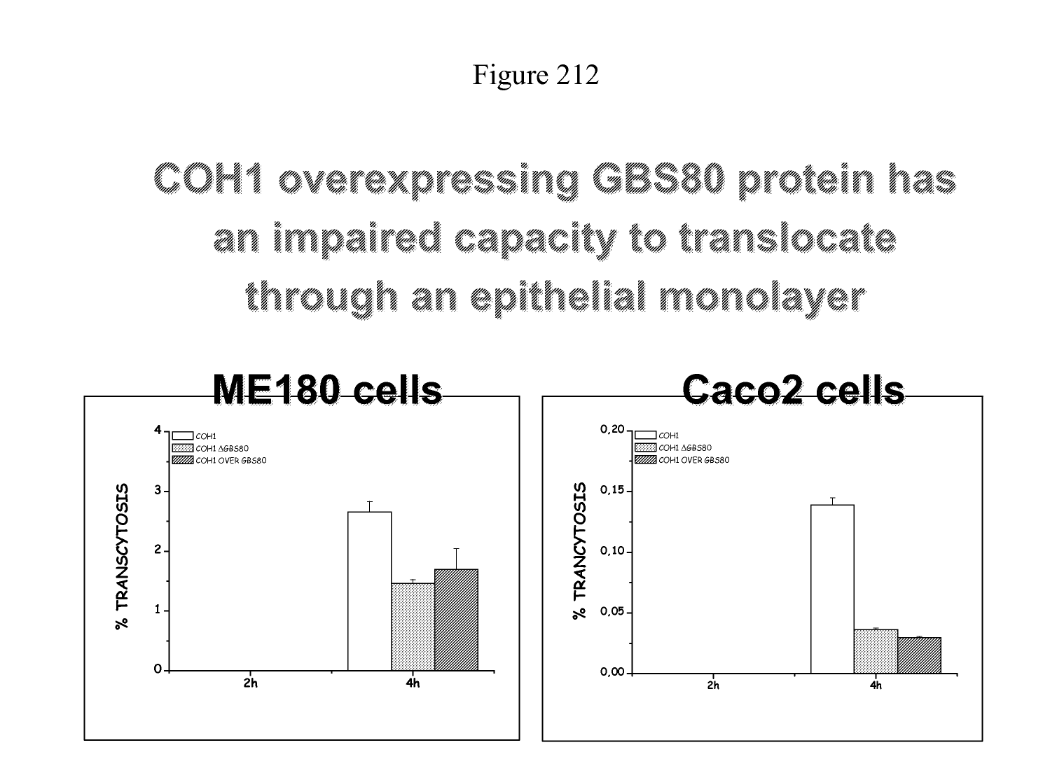

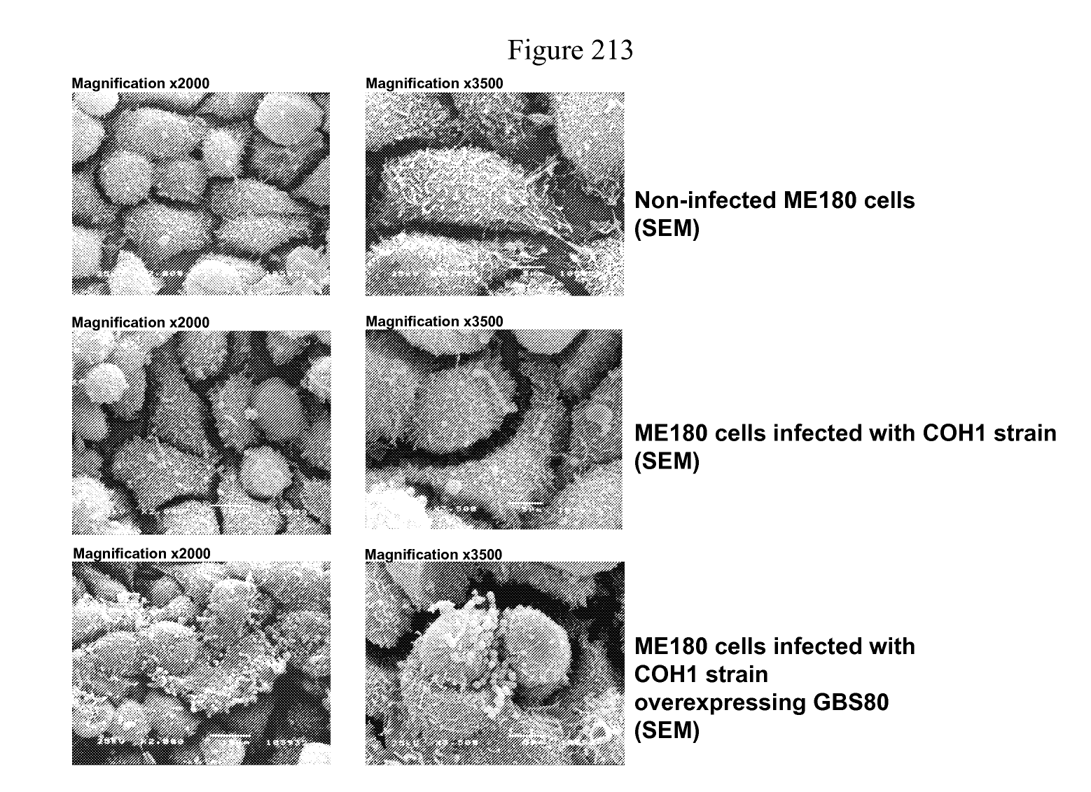

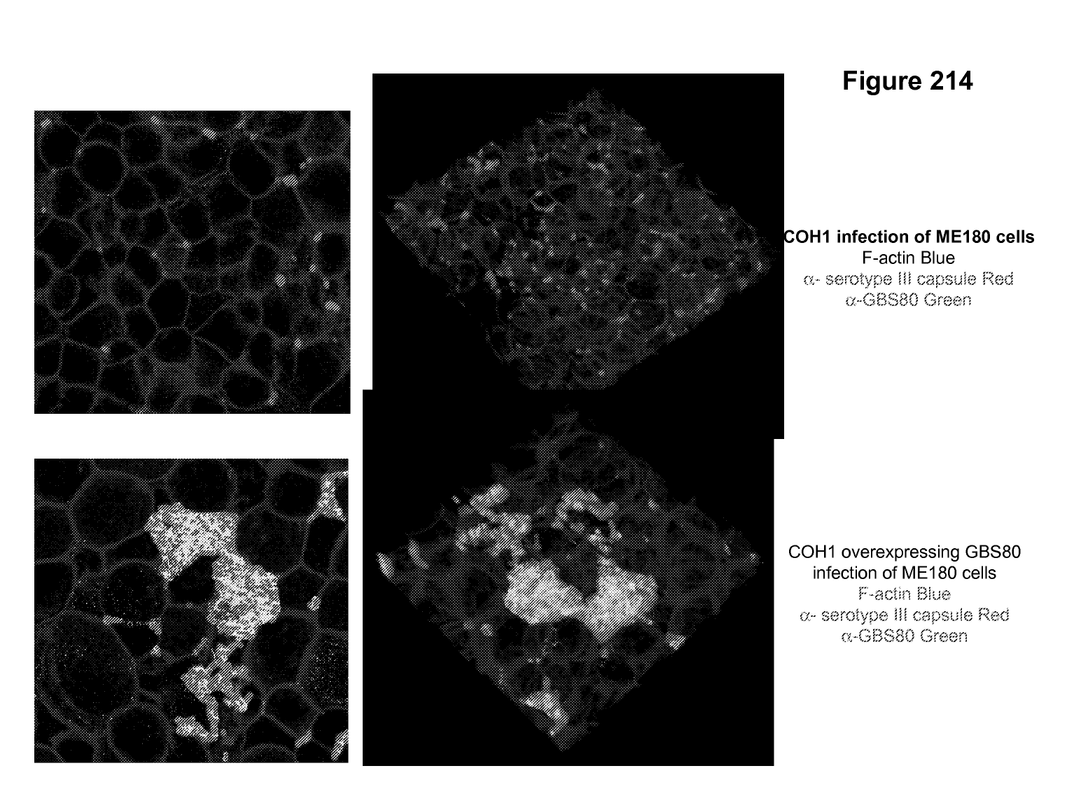

GBS 80 may also be involved in formation of biofilms. COH1 bacteria overexpressing GBS 80 protein have an impaired ability to translocate through an epithelial monolayer. See FIG. 212. These COH1 bacteria overexpressing GBS 80 form microcolonies on epithelial cells. See FIGS. 213 and 214. These microcolonies may be the initiation of biofilm development.

AI Surface proteins may also demonstrate functional homology to previously identified adhesion proteins or extracellular matrix (ECM) binding proteins. For example, GBS 80, a surface protein in AI-1, exhibits some functional homology to FimA, a major fimbrial subunit of a Gram positive bacteria A. naeslundii. FimA is thought to be involved in binding salivary proteins and may be a component in a fimbrae on the surface of A. naeslundii. See Yeung et al. (1997) Infection & Immunity 65:2629-2639; Yeunge et al (1998) J. Bacteriol 66:1482-1491; Yeung et al. (1988) J. Bacteriol 170:3803-3809; and Li et al. (2001) Infection & Immunity 69:7224-7233.

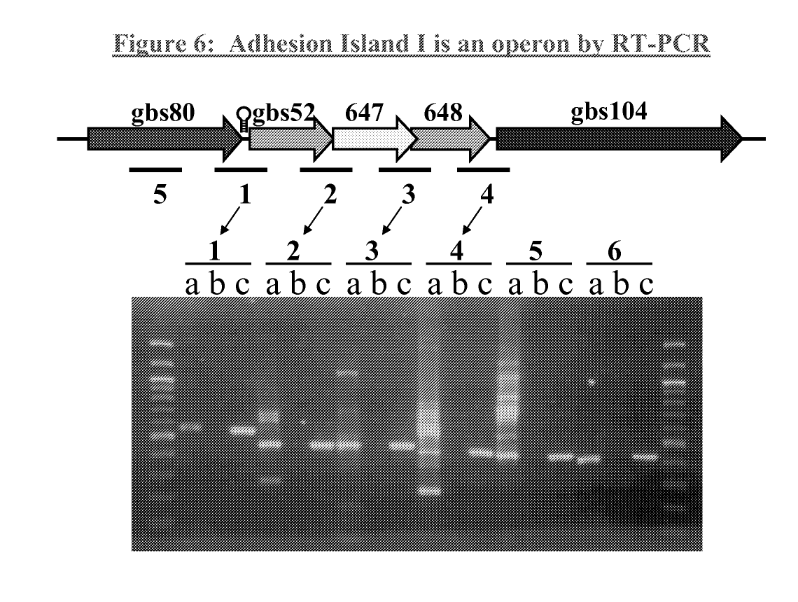

A similar functional homology has also been identified between GBS 80 and proteins involved in pili formation in the Gram positive bacteria Corynebacterium diphtheriae (SpaA, SpaD, and SpaH). See, Ton-That et al. (2003) Molecular Microbiology 50(4):1429-1438 and Ton-That et al. (2004) Molecular Microbiology 53(1):251-261. The C. diphtheriae proteins all included a pilin motif of WxxxVxVYPK (SEQ ID NO:151; where x indicates a varying amino acid residue). The lysine (K) residue is particularly conserved in the C. diphtheriae pilus proteins and is thought to be involved in sortase catalyzed oligomerization of the subunits involved in the C. diphtheriae pilus structure. (The C. diphtheriae pilin subunit SpaA is thought to occur by sortase-catalyzed amide bond cross-linking of adjacent pilin subunits. As the thioester-linked acyl intermediate of sortase requires nucleophilic attack for release, the conserved lysine within the SpaA pilin motif might function as an amino group acceptor of cleaved sorting signals, thereby providing for covalent linkages of the C. diphtheria pilin subunits. See FIG. 6(d) of Ton-That et al., Molecular Microbiology (2003) 50(4):1429-1438.)

In addition, an “E box” comprising a conserved glutamic acid residue has also been identified in the C. diphtheria pilin associated proteins as important in C. diphtheria pilin assembly. The E box motif generally comprises YxLxETxAPxGY (SEQ ID NO:152; where x indicates a varying amino acid residue). In particular, the conserved glutamic acid residue within the E box is thought necessary for C. diphtheria pilus formation.

Preferably, the AI-1 polypeptides of the immunogenic compositions comprise an E box motif. Some examples of E box motifs in the AI-1 polypeptides may include the amino acid sequences YxLxExxxxxGY (SEQ ID NO:153), YxLxExxxPxGY (SEQ ID NO:154), or YxLxETxAPxGY (SEQ ID NO:152). Specifically, the E box motif of the polypeptides may comprise the amino acid sequences YKLKETKAPEGY (SEQ ID NO:155), YVLKEIETQSGY (SEQ ID NO:156), or YKLYEISSPDGY (SEQ ID NO:157).

As discussed in more detail below, a pilin motif containing a conserved lysine residue and an E box motif containing a conserved glutamic acid residue have both been identified in GBS 80.

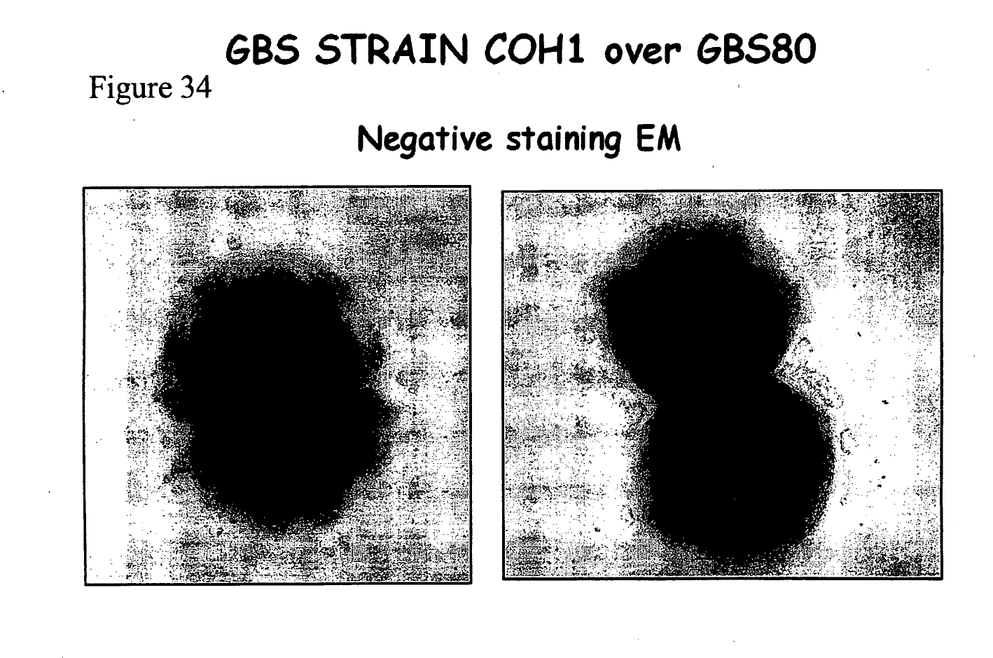

While previous publications have speculated that pilus-like structures might be formed on the surface of streptococci, (see, e.g., Ton-That et al., Molecular Microbiology (2003) 50(4): 1429-1438), these structures have not been previously visible in negative stain (non-specific) electron micrographs, throwing such speculations into doubt. For example, FIG. 34 presents electron micrographs of GBS serotype III, strain isolate COH1 with a plasmid insert to facilitate the overexpression of GBS 80. This EM photo was produced with a standard negative stain-no pilus structures are distinguishable. In addition, the use of such AI surface proteins in immunogenic compositions for the treatment or prevention of infection against a Gram positive bacteria has not been previously described.

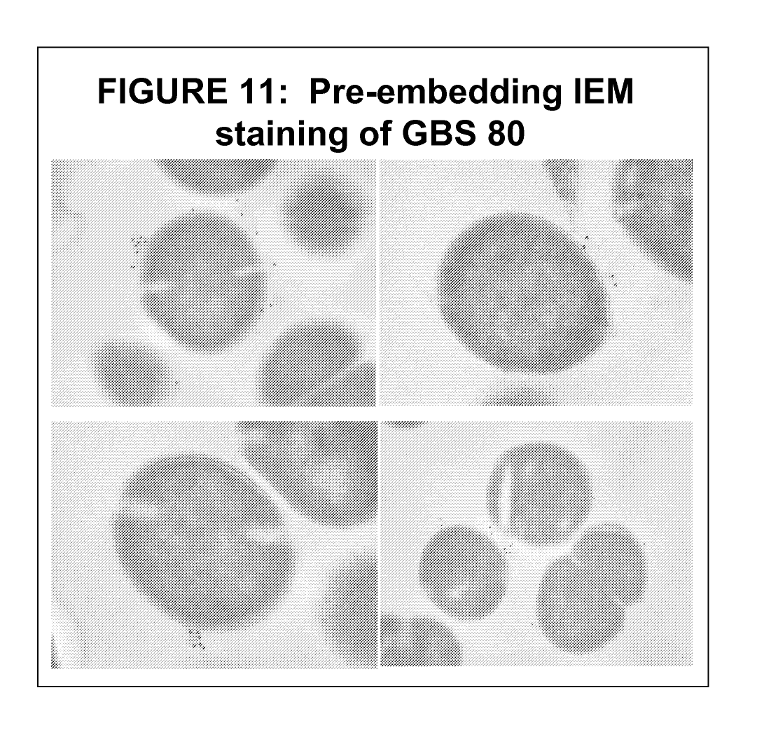

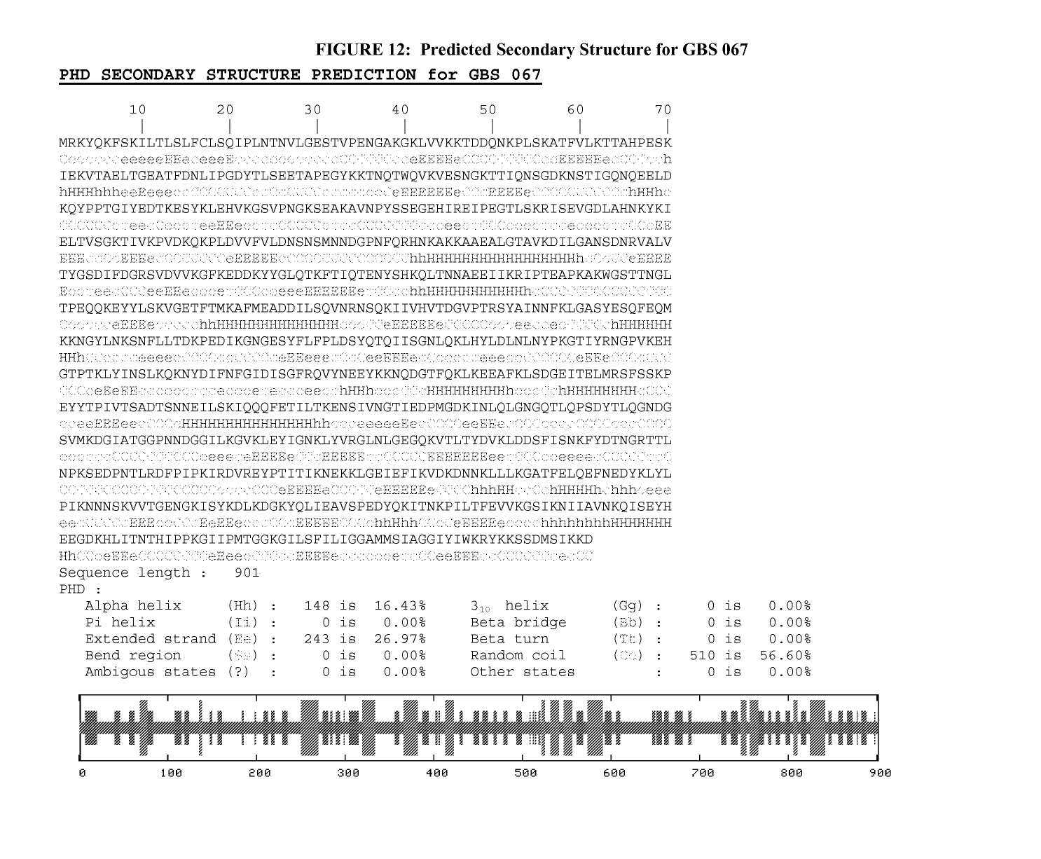

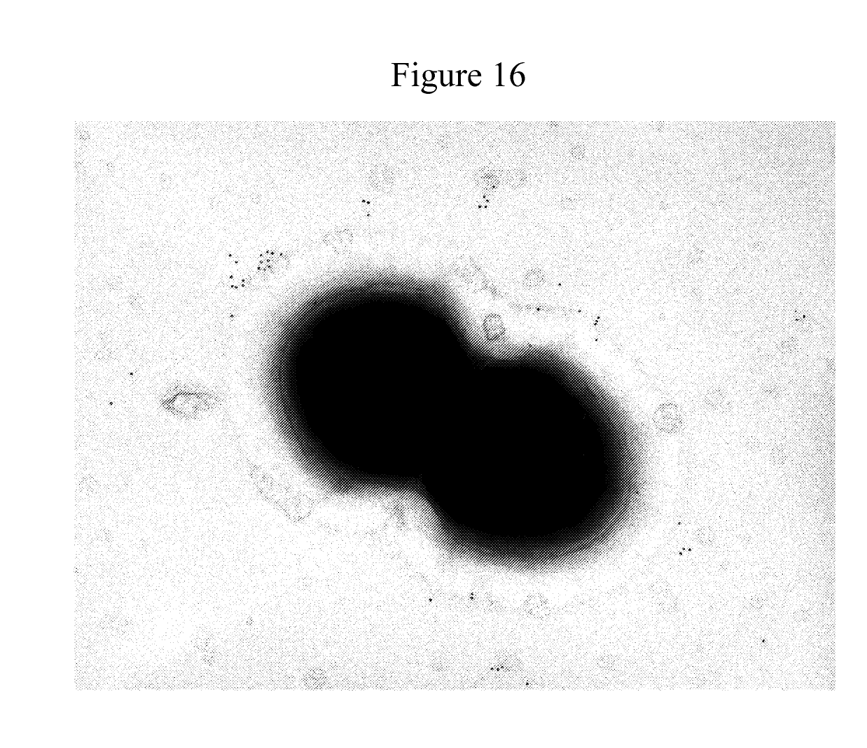

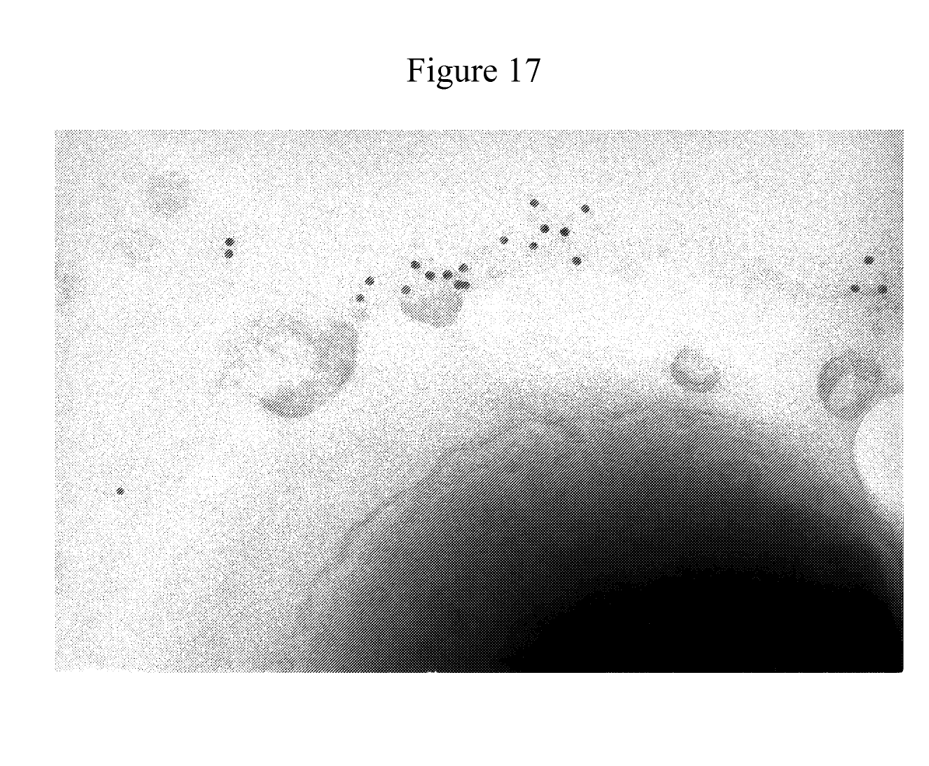

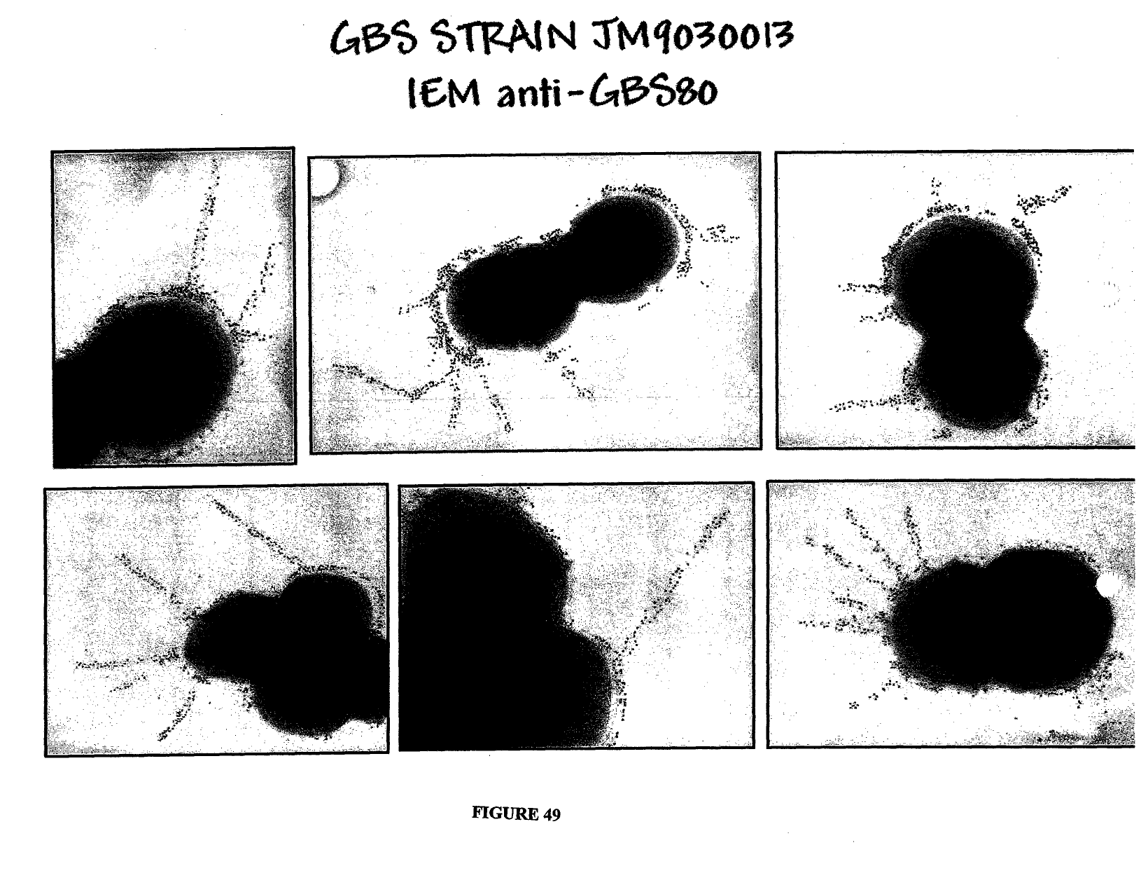

Surprisingly, Applicants have now identified the presence of GBS 80 in surface exposed pilus formations visible in electron micrographs. These structures are only visible when the electron micrographs are specifically stained against an AI surface protein such as GBS 80. Examples of these electron micrographs are shown in FIGS. 11, 16 and 17, which reveal the presence of pilus structures in wild type COH1 Streptococcus agalactiae. Other examples of these electron micrographs are shown in FIG. 49, which reveals that GBS 80 is associated with pili in a wild type clinical isolate of S. agalactiae, JM9030013. (See FIG. 49.)

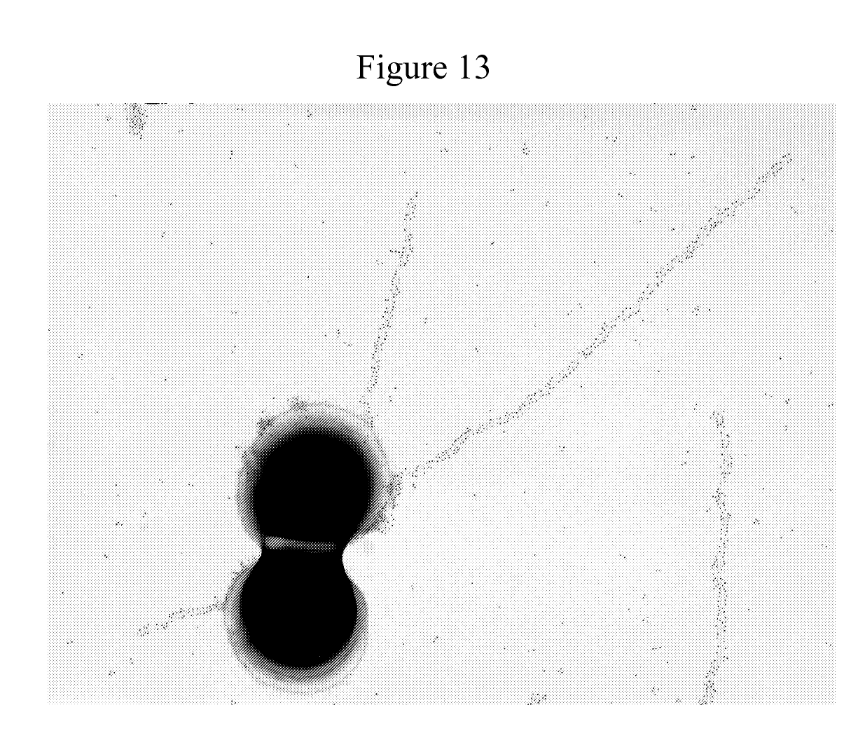

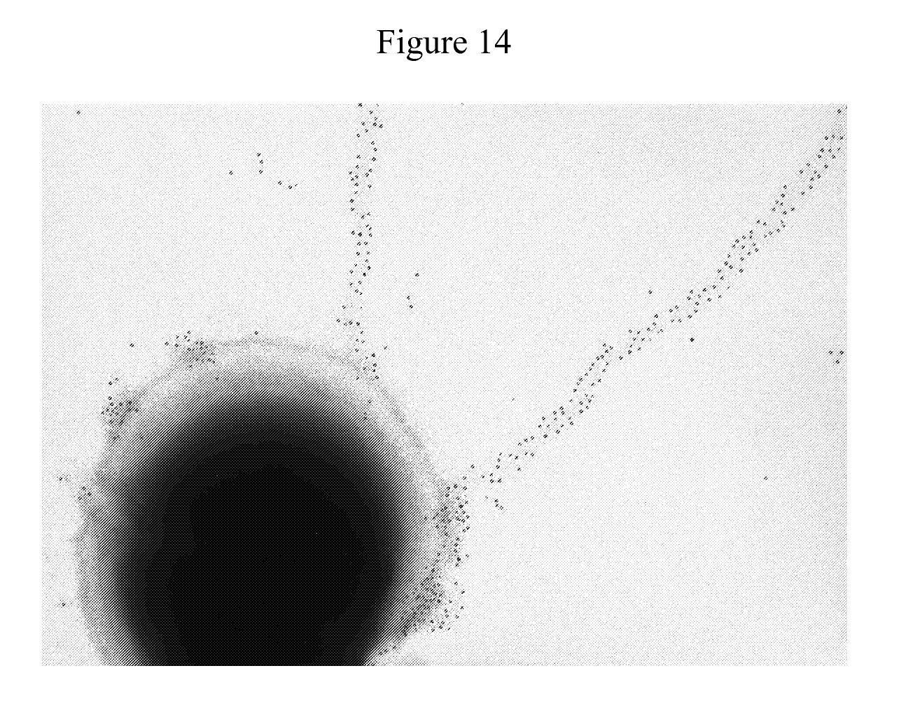

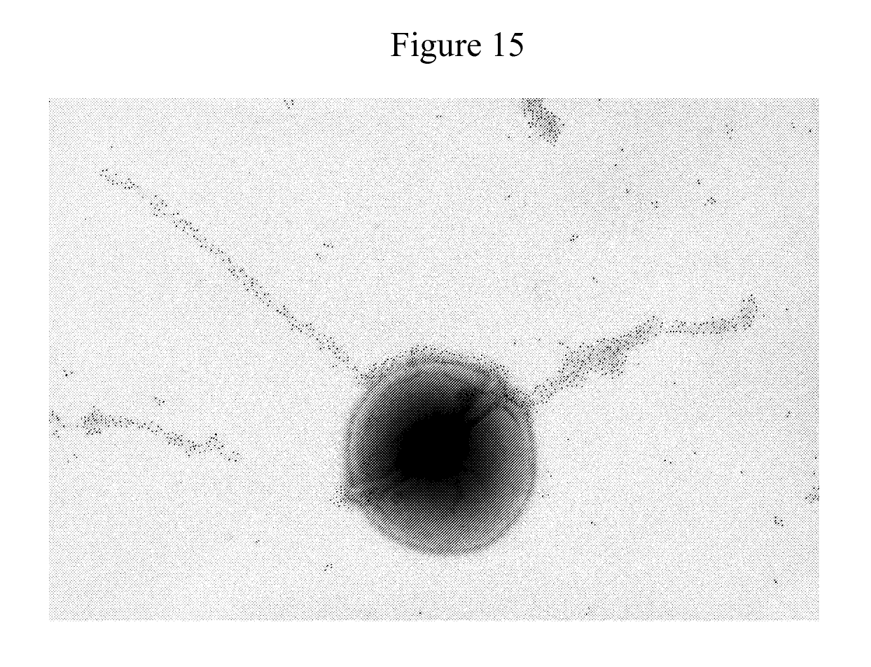

Applicants have also constructed mutant GBS strains containing a plasmid comprising the GBS 80 sequence resulting in the overexpression of GBS 80 within this mutant. The electron micrographs of FIGS. 13-15 are also stained against GBS 80 and reveal long, oligomeric structures containing GBS 80 which appear to cover portions of the surface of the bacteria and stretch far out into the supernatant.

In some instances, the formation of pili structures on GBS appears to be correlated to surface expression of GBS 80. FIG. 61 provides FAC analysis of GBS 80 surface levels on bacterial strains COH1 and JM9130013 using an anti-GBS 80 antisera Immunogold electron microscopy of the COH1 and JM9130013 bacteria using anti-GBS 80 antisera demonstrates that JM9130013 bacteria, which have higher values for GBS 80 surface expression, also form longer pili structures.

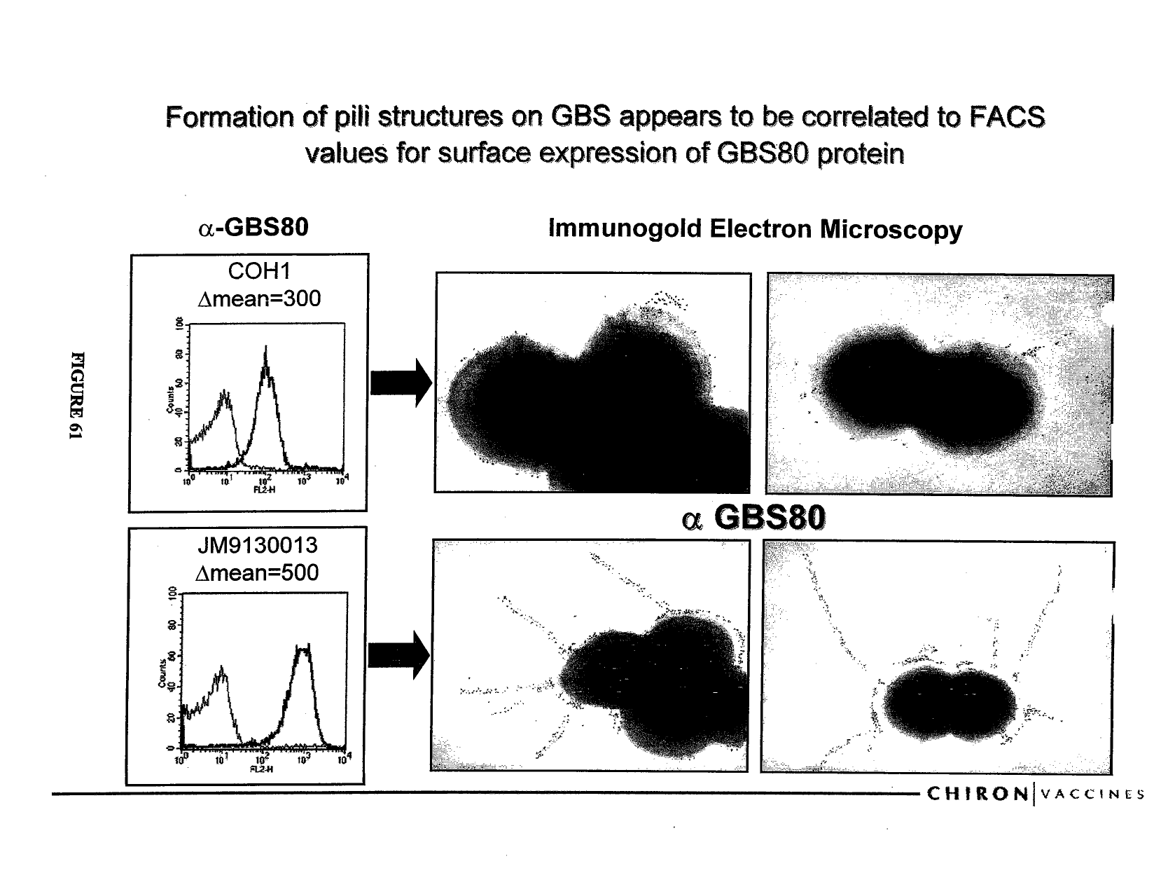

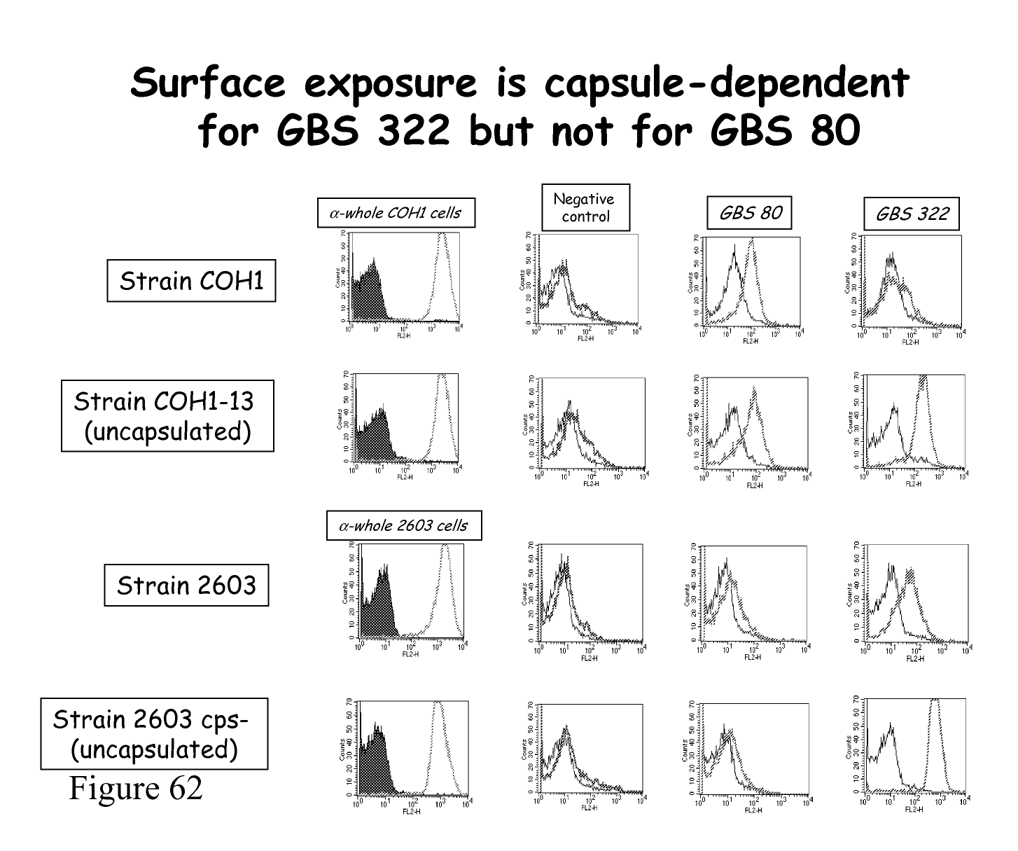

The surface exposure of GBS 80 on GBS is generally not capsule-dependent. FIG. 62 provides FACS analysis of capsulated and uncapsulated GBS analyzed with anti-GBS 80 and anti-GBS 322 antibodies. Surface exposure of GBS 80, unlike GBS 322, is not capsule dependent.

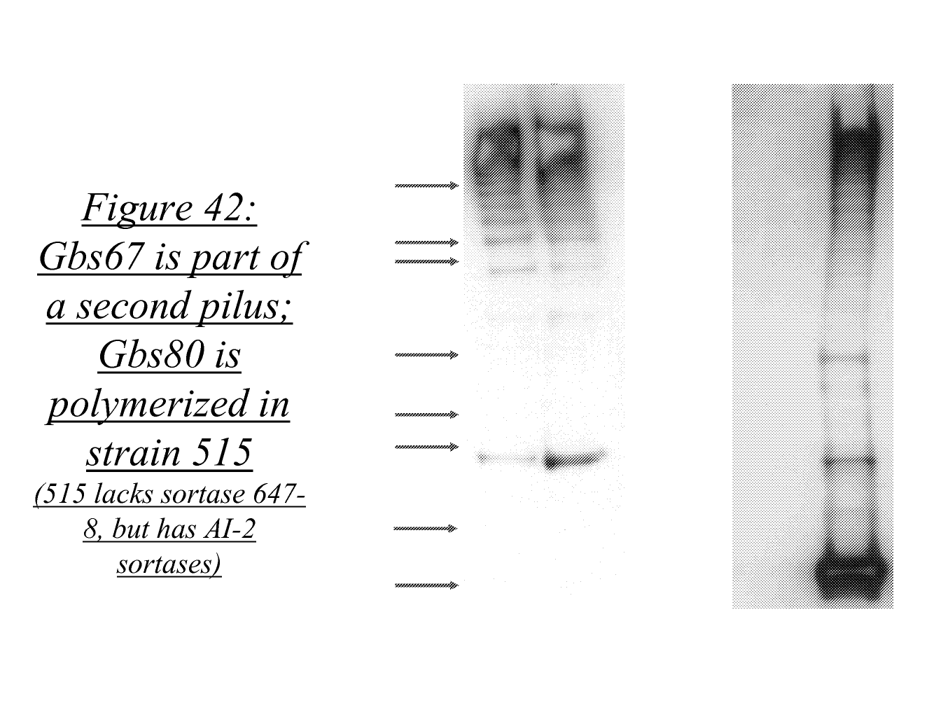

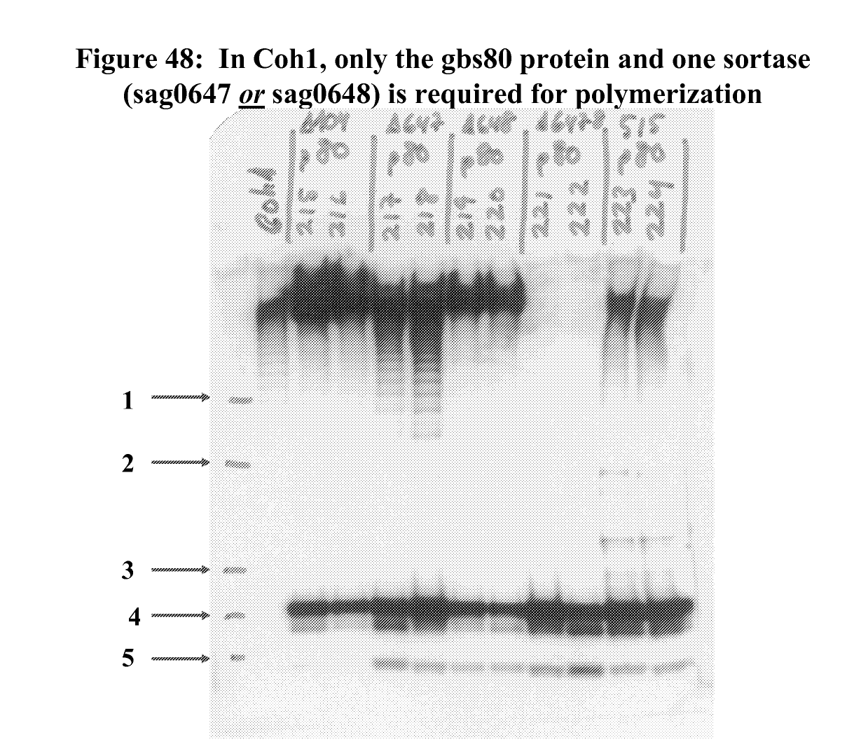

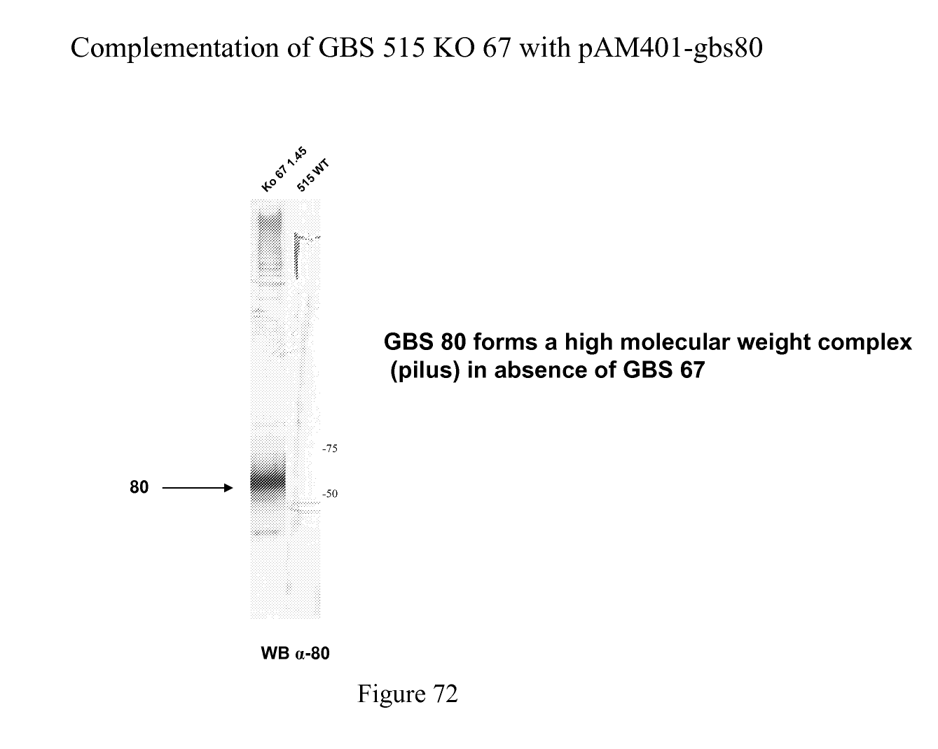

An Adhesin Island surface protein, such as GBS 80 appears to be required for pili formation, as well as an Adhesin Island sortase. Pili are formed in Cohl bacterial clones that overexpress GBS 80, but lack GBS 104, or one of the AI-1 sortases sag0647 or sag0648. However, pili are not formed in Cohl bacterial clones that overexpress GBS 80 and lack both sag0647 and sag0648. Thus, for example, it appears that at least GBS 80 and a sortase, sag0647 or sag0648, may be necessary for pili formation. (See FIG. 48.) Overexpression of GBS 80 in GBS strain 515, which lacks an AI-1, also assembles GBS 80 into pili. GBS strain 515 contains an AI-2, and thus AI-2 sortases. The AI-2 sortases in GBS strain 515 apparently polymerize GBS 80 into pili. (See FIG. 42.) Overexpression of GBS 80 in GBS strain 515 cell knocked out for GBS 67 expression also apparently polymerizes GBS 80 into pili. (See FIG. 72.)

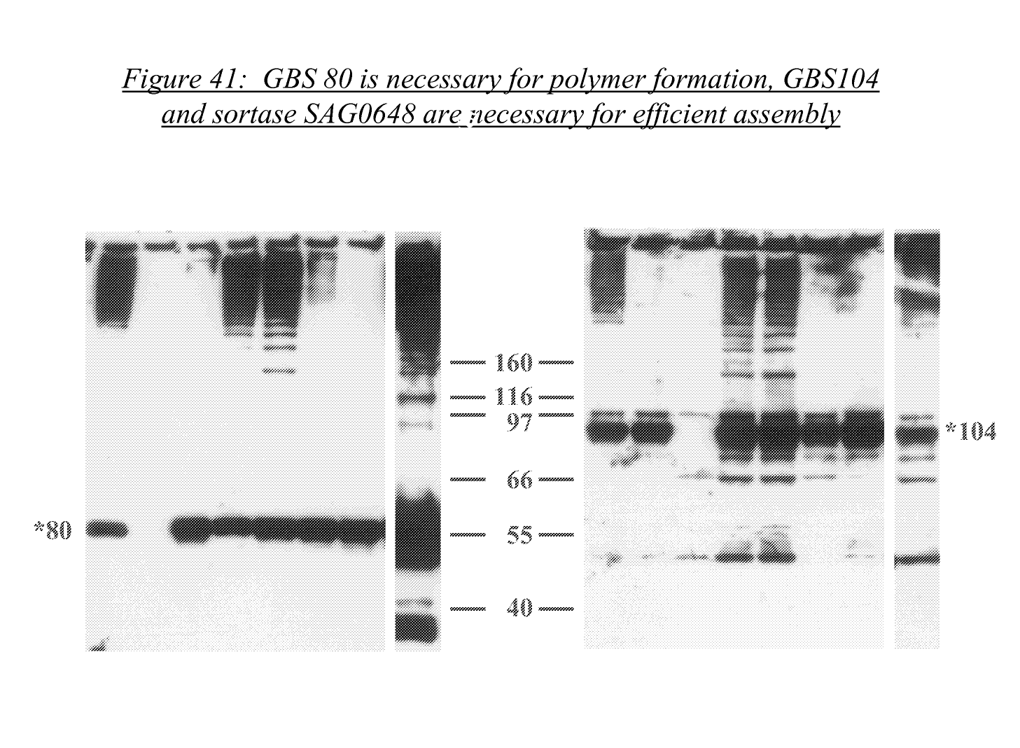

While GBS 80 appears to be required for GBS AI-1 pili formation, GBS 104 and sortase SAG0648 appears to be important for efficient AI-1 pili assembly. For example, high-molecular structures are not assembled in isogenic COH1 strains which lack expression of GBS 80 due to gene disruption and are less efficiently assembled in isogenic COH1 strains which lack the expression of GBS 104 (see FIG. 41). This GBS strain comprises high molecular weight pili structures composed of covalently linked GBS 80 and GBS 104 subunits. In addition, deleting SAG0648 in COH1 bacteria interferes with assembly of some of the high molecular weight pili structures. Thus, indicating that SAG0648 plays a role in assembly of these pilin species. (See FIG. 41).

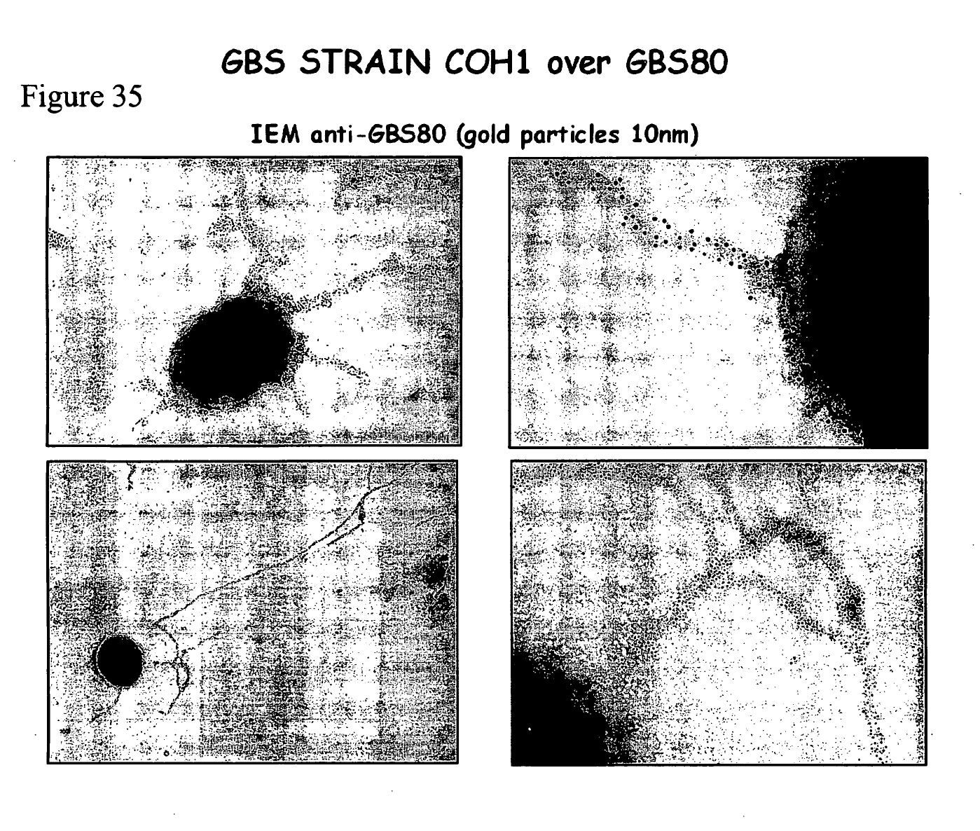

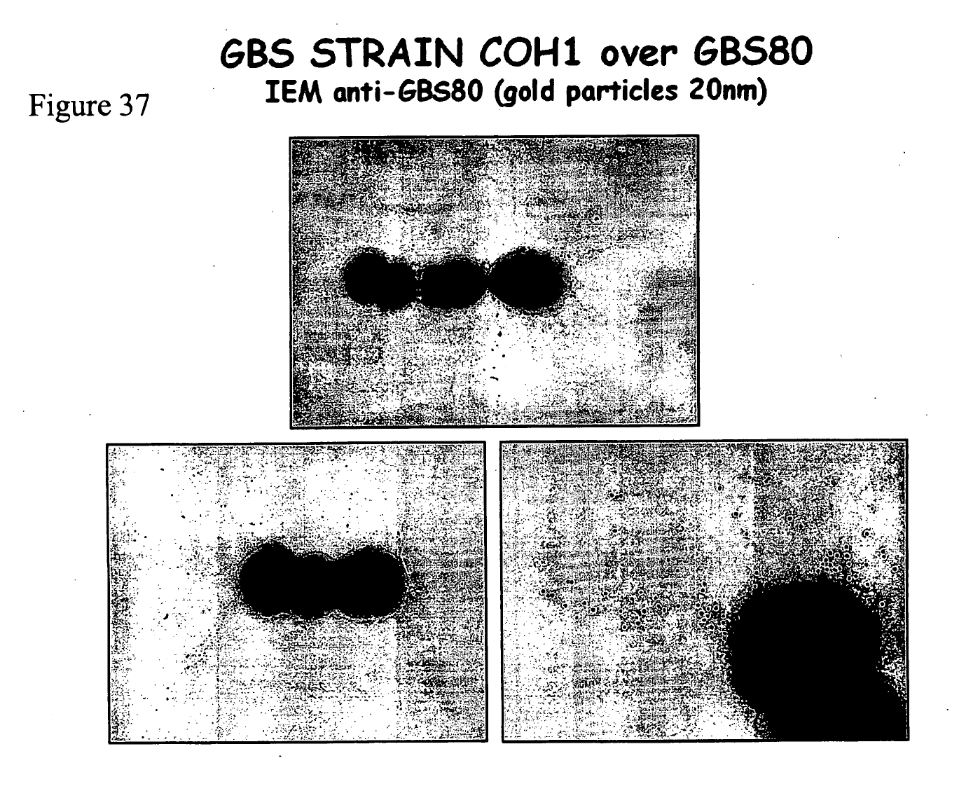

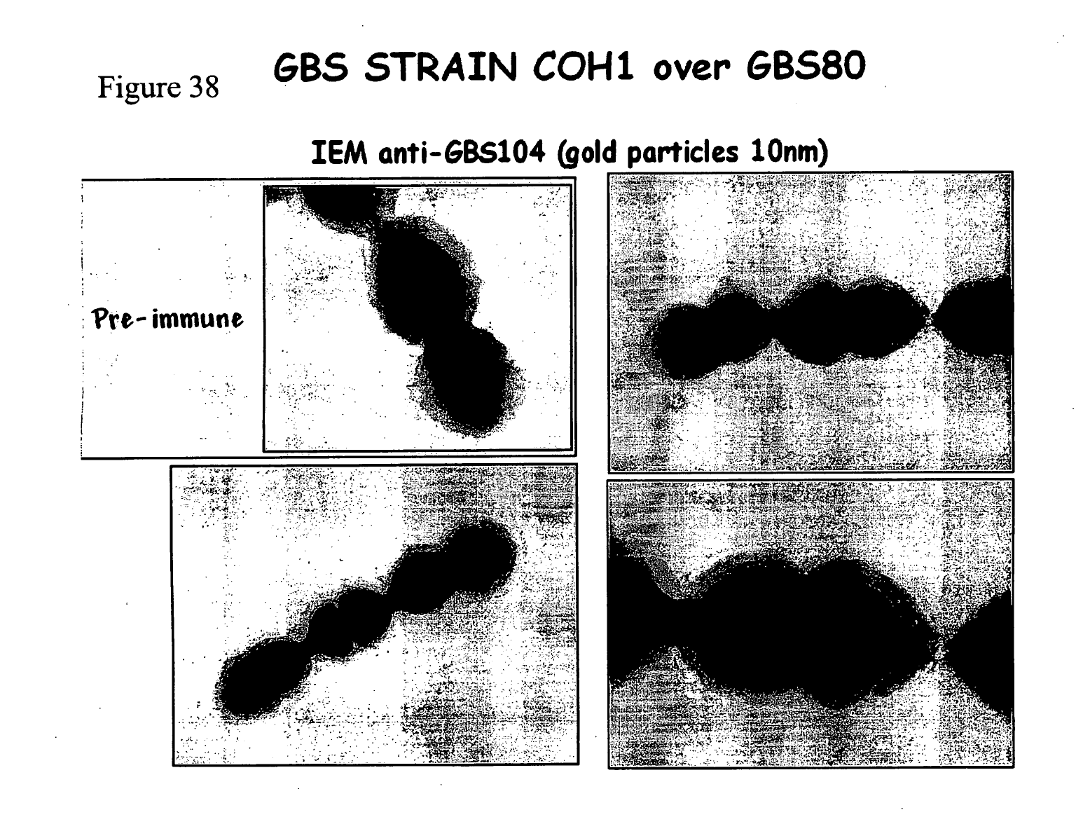

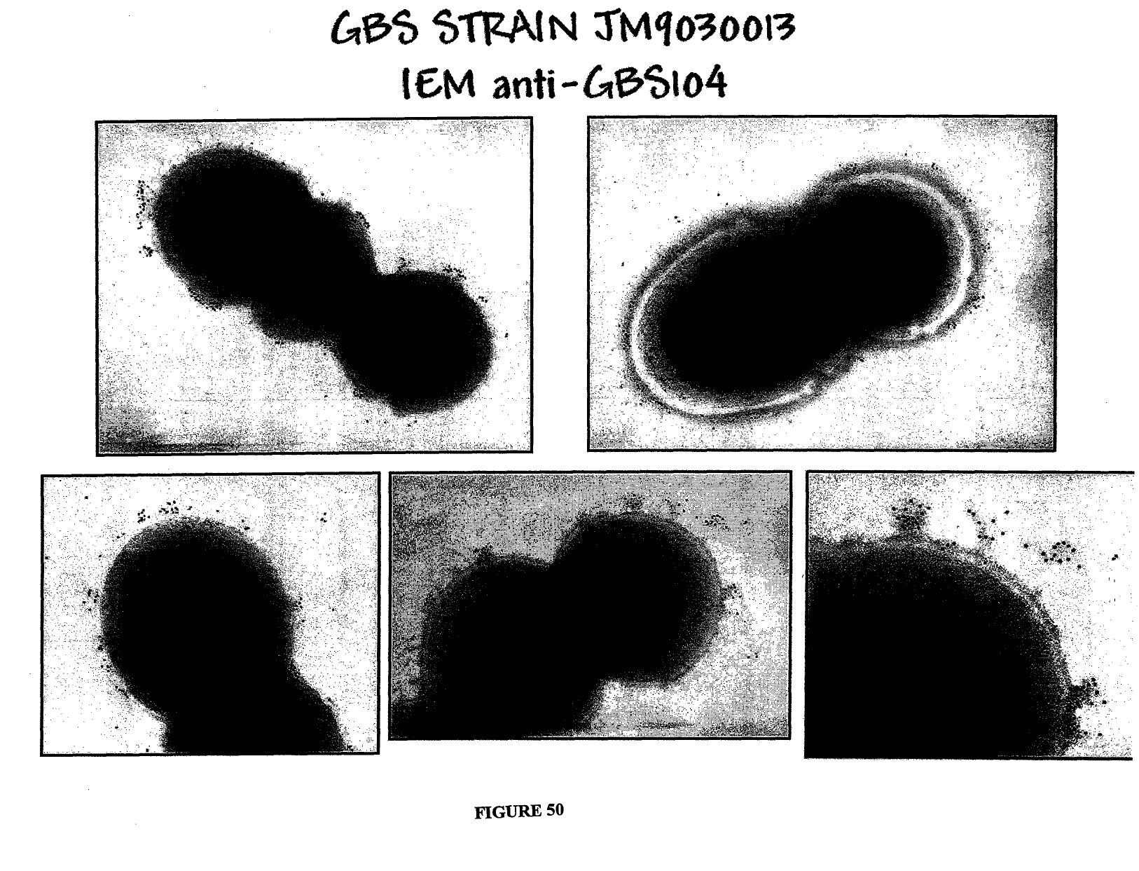

EM photos confirm the involvement of AI surface protein GBS 104 within the hyperoligomeric structures of a GBS strain adapted for increased GBS 80 expression. (See FIGS. 34-41 and Example 6). In a wild type serotype VIII GBS strain, strain JM9030013, IEM identifies GBS 104 as forming clusters on the bacterial surface. (See FIG. 50.)

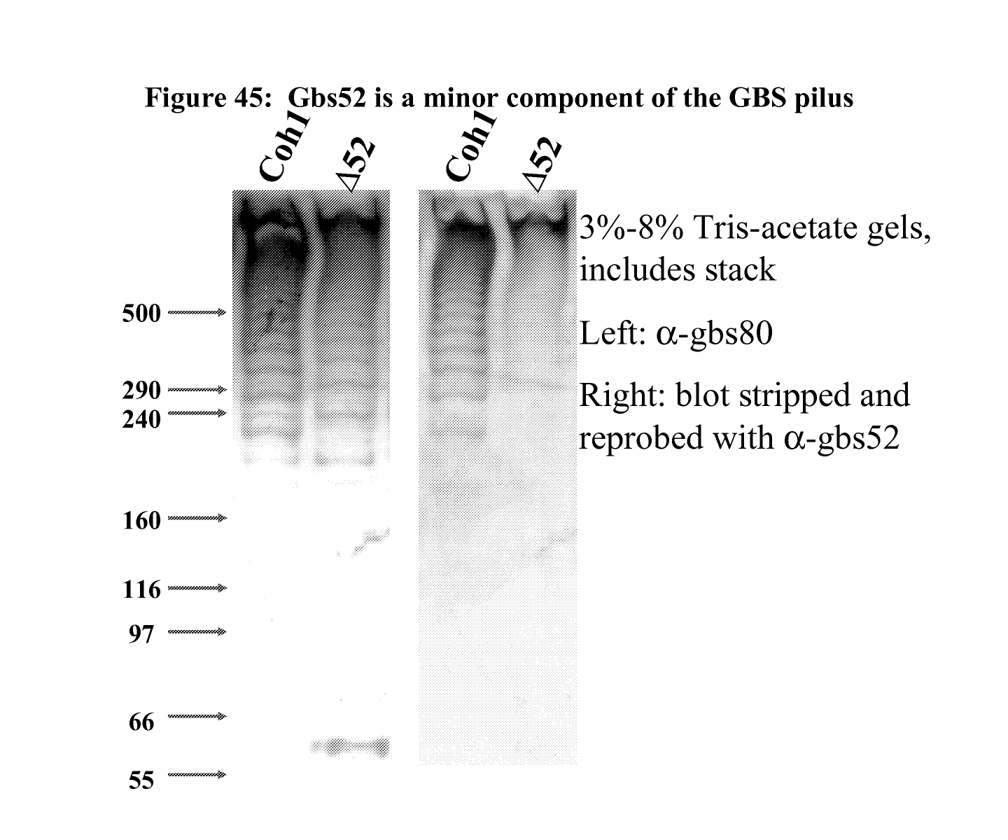

GBS 52 also appears to be a component of the GBS pili Immunoblots using an anti-GBS 80 antisera on total cell extracts of Cohl and a GBS 52 null mutant Cohl reveal a shift in detected proteins in the Cohl wild type strain relative to the GBS 52 null mutant Cohl strain. The shifted proteins were also detected in the wild type Cohl bacteria with an anti-GBS 52 antisera, indicating that the GBS 52 may be present in the pilus. (See FIG. 45.)

In one embodiment, the invention includes a composition comprising oligomeric, pilus-like structures comprising an AI surface protein such as GBS 80. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine amino acid residue.

AI surface proteins or fragments thereof to be incorporated into the oligomeric, pilus-like structures of the invention will preferably include one or both of a pilin motif comprising a conserved lysine residue and an E box motif comprising a conserved glutamic acid residue.

More than one AI surface protein may be present in the oligomeric, pilus-like structures of the invention. For example, GBS 80 and GBS 104 may be incorporated into an oligomeric structure. Alternatively, GBS 80 and GBS 52 may be incorporated into an oligomeric structure, or GBS 80, GBS 104 and GBS 52 may be incorporated into an oligomeric structure.

In another embodiment, the invention includes compositions comprising two or more AI surface proteins. The composition may include surface proteins from the same adhesin island. For example, the composition may include two or more GBS AI-1 surface proteins, such as GBS 80, GBS 104 and GBS 52. The surface proteins may be isolated from Gram positive bacteria or they may be produced recombinantly.

The oligomeric, pilus like structures may be used alone or in the combinations of the invention. In one embodiment, the invention comprises a GBS Adhesin Island protein in oligomeric form, preferably in a hyperoligomeric form. In one embodiment, the invention comprises a composition comprising one or more GBS Adhesin Island 1 (“AI-1”) proteins and one or more GBS Adhesin Island 2 (“AI-2”) proteins, wherein one or more of the Adhesin Island proteins is in the form of an oligomer, preferably in a hyperoligomeric form.

The oligomeric, pilus-like structures of the invention may be combined with one or more additional GBS proteins. In one embodiment, the oligomeric, pilus-like structures comprise one or more AI surface proteins in combination with a second GBS protein. The second GBS protein may be a known GBS antigen, such as GBS 322 (commonly referred to as “sip”) or GBS 276. Nucleotide and amino acid sequences of GBS 322 sequenced from serotype V isolated strain 2603 V/R are set forth in WO 02/35771 as SEQ ID 8539 and SEQ ID 8540 and in the present specification as SEQ ID NOS: 38 and 39. A particularly preferred GBS 322 polypeptide lacks the N-terminal signal peptide, amino acid residues 1-24. An example of a preferred GBS 322 polypeptide is a 407 amino acid fragment and is shown in SEQ ID NO:40. Examples of preferred GBS 322 polypeptides are further described in WO 2005/028618.

Additional GBS proteins which may be combined with the GBS AI surface proteins of the invention are also described in WO 2005/028618. These GBS proteins include GBS 91, GBS 184, GBS 305, GBS 330, GBS 338, GBS 361, GBS 404, GBS 690, and GBS 691.

Additional GBS proteins which may be combined with the GBS AI surface proteins of the invention are described in WO 02/34771. These GBS proteins include but are not limited to GBS293, GBS65, GBS97, GBS84, GBS147, and GBS325.

GBS polysaccharides which may be combined with the GBS AI surface proteins of the invention are described in WO 2004/041157. For example, the GBS AI surface proteins of the invention may be combined with a GBS polysaccharides selected from the group consisting of serotype Ia, Ib, Ia/c, II, III, IV, V, VI, VII and VIII.

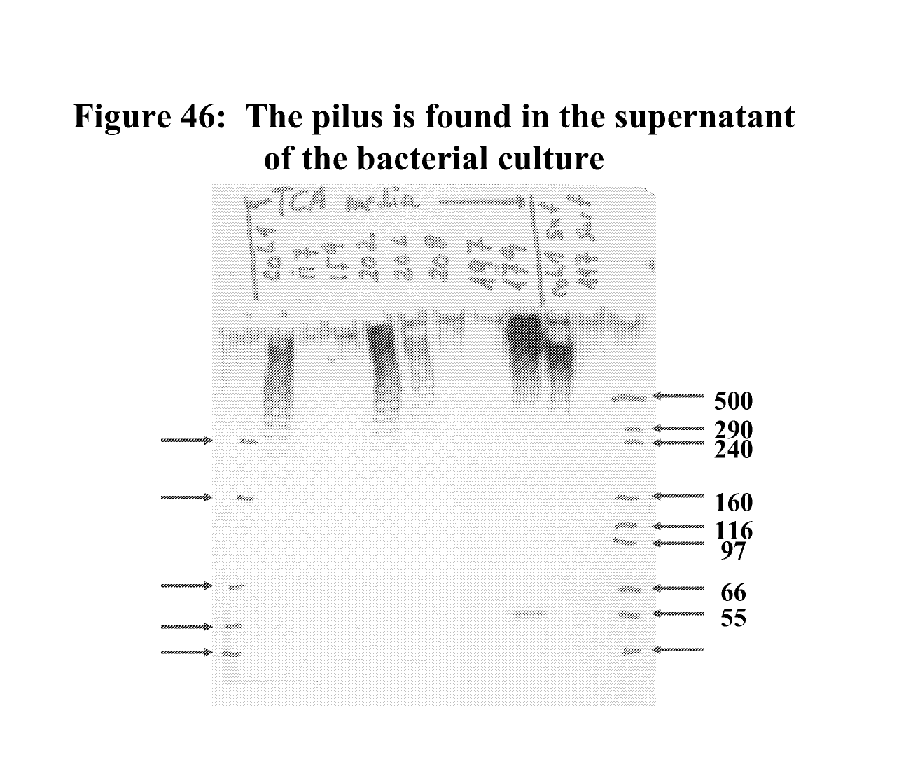

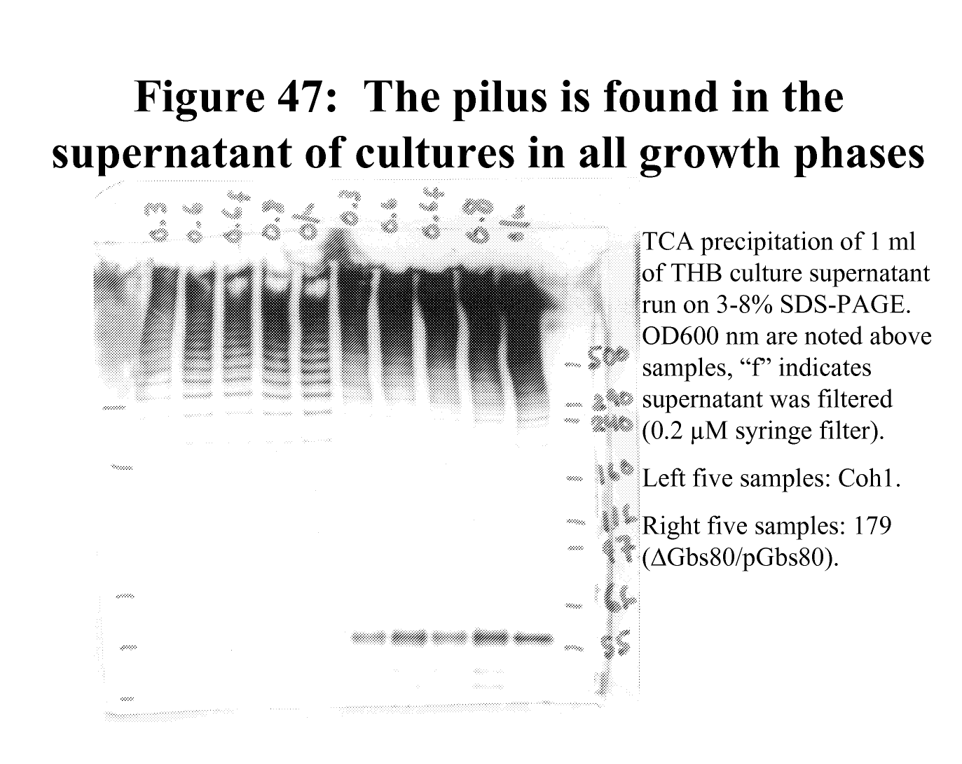

The oligomeric, pilus-like structures may be isolated or purified from bacterial cultures in which the bacteria express an AI surface protein. The invention therefore includes a method for manufacturing an oligomeric AI surface antigen comprising culturing a GBS bacterium that expresses the oligomeric AI protein and isolating the expressed oligomeric AI protein from the GBS bacteria. The AI protein may be collected from secretions into the supernatant or it may be purified from the bacterial surface. The method may further comprise purification of the expressed AI protein. Preferably, the AI protein is in a hyperoligomeric form. Macromolecular structures associated with oligomeric pili are observed in the supernatant of cultured GBS strain Cohl. (See FIG. 46.) These pili are found in the supernatant at all growth phases of the cultured Cohl bacteria. (See FIG. 47.)

The oligomeric, pilus-like structures may be isolated or purified from bacterial cultures overexpressing an AI surface protein. The invention therefore includes a method for manufacturing an oligomeric Adhesin Island surface antigen comprising culturing a GBS bacterium adapted for increased AI protein expression and isolation of the expressed oligomeric Adhesin Island protein from the GBS bacteria. The AI protein may be collected from secretions into the supernatant or it may be purified from the bacterial surface. The method may further comprise purification of the expressed Adhesin Island protein. Preferably, the Adhesin Island protein is in a hyperoligomeric form.

The GBS bacteria are preferably adapted to increase AI protein expression by at least two (e.g., 2, 3, 4, 5, 8, 10, 15, 20, 25, 30, 35, 40, 45, 50, 60, 70, 80, 90, 100, 125, 150 or 200) times wild type expression levels.

GBS bacteria may be adapted to increase AI protein expression by any means known in the art, including methods of increasing gene dosage and methods of gene upregulation. Such means include, for example, transformation of the GBS bacteria with a plasmid encoding the AI protein. The plasmid may include a strong promoter or it may include multiple copies of the sequence encoding the AI protein. Optionally, the sequence encoding the AI protein within the GBS bacterial genome may be deleted. Alternatively, or in addition, the promoter regulating the GBS Adhesin Island may be modified to increase expression.

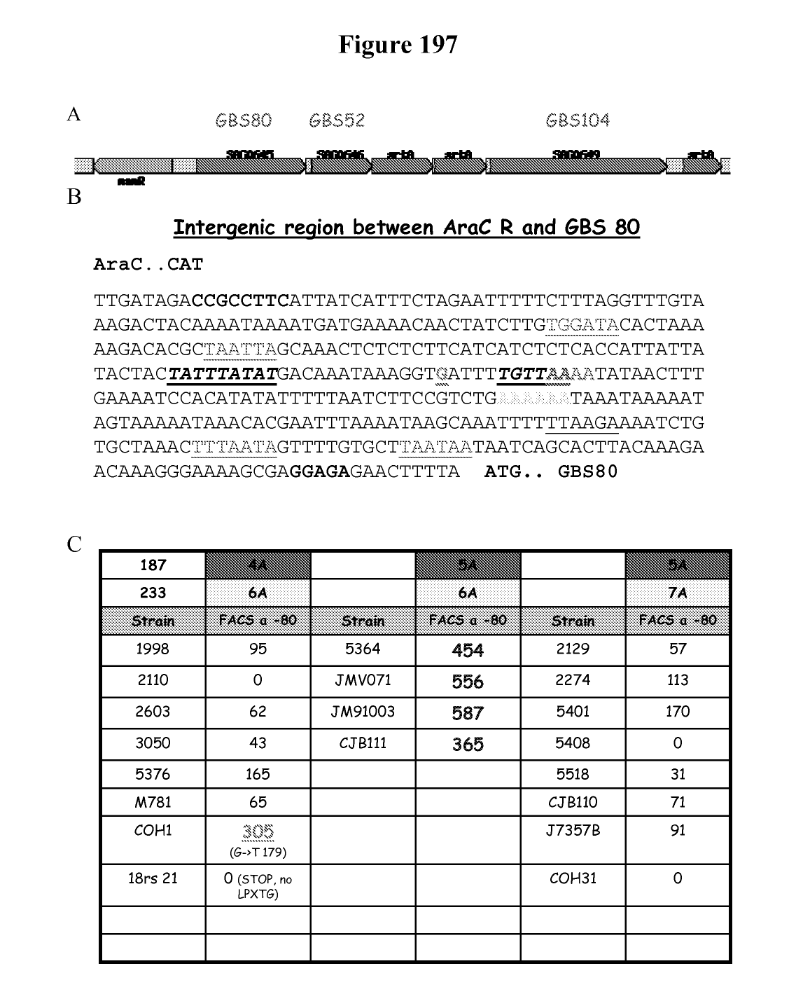

GBS bacteria harbouring a GBS AI-1 may also be adapted to increase AI protein expression by altering the number adenosine nucleotides present at two sites in the intergenic region between AraC and GBS 80. See FIG. 197A, which is a schematic showing the organization of GBS AI-1 and FIG. 197B, which provides the sequence of the intergenic region between AraC and GBS 80 in the AI. The adenosine tracts which applicants have identified as influencing GBS 80 surface expression are at nucleotide positions 187 and 233 of the sequence shown in FIG. 197B (SEQ ID NO:273). Applicants determined the influence of these adenosine tracts on GBS 80 surface expression in strains of GBS bacteria harboring four adenosines at position 187 and six adenosines at position 233, five adenosines at position 187 and six adenosines position 233, and five adenosines at position 187 and seven adenosines at position 233. FACS analysis of these strains using anti GBS 80 antiserum determined that an intergenic region with five adenosines at position 187 and six adenosines at position 233 had higher expression levels of GBS 80 on their surface than other stains. See FIG. 197C for results obtained from the FACS analysis. Therefore, manipulating the number of adenosines present at positions 187 and 233 of the AraC and GBS 80 intergenic region may further be used to adapt GBS to increase AI protein expression.

The invention further includes GBS bacteria which have been adapted to produce increased levels of AI surface protein. In particular, the invention includes GBS bacteria which have been adapted to produce oligomeric or hyperoligomeric AI surface protein, such as GBS 80. In one embodiment, the Gram positive bacteria of the invention are inactivated or attenuated to permit in vivo delivery of the whole bacteria, with the AI surface protein exposed on its surface.

The invention further includes GBS bacteria which have been adapted to have increased levels of expressed AI protein incorporated in pili on their surface. The GBS bacteria may be adapted to have increased exposure of oligomeric or hyperoligomeric AI proteins on its surface by increasing expression levels of a signal peptidase polypeptide. Increased levels of a local signal peptidase expression in Gram positive bacteria (such us LepA in GAS) are expected to result in increased exposure of pili proteins on the surface of Gram positive bacteria. Increased expression of a leader peptidase in GBS may be achieved by any means known in the art, such as increasing gene dosage and methods of gene upregulation. The GBS bacteria adapted to have increased levels of leader peptidase may additionally be adapted to express increased levels of at least one pili protein.

Alternatively, the AI proteins of the invention may be expressed on the surface of a non-pathogenic Gram positive bacteria, such as Streptococcus gordonii (See, e.g., Byrd et al., “Biological consequences of antigen and cytokine co-expression by recombinant Streptococcus gordonii vaccine vectors, ” Vaccine (2002) 20:2197-2205) or Lactococcus lactis (See, e.g., Mannam et al., “Mucosal Vaccine Made from Live, Recombinant Lactococcus lactis Protects Mice against Pharyngeal Infection with Streptococcus pyogenes” Infection and Immunity (2004) 72(6):3444-3450). As used herein, non-pathogenic Gram positive bacteria refer to Gram positive bacteria which are compatible with a human host subject and are not associated with human pathogenesis. Preferably, the non-pathogenic bacteria are modified to express the AI surface protein in oligomeric, or hyper-oligomeric form. Sequences encoding for an AI surface protein and, optionally, an AI sortase, may be integrated into the non-pathogenic Gram positive bacterial genome or inserted into a plasmid. The non-pathogenic Gram positive bacteria may be inactivated or attenuated to facilitate in vivo delivery of the whole bacteria, with the AI surface protein exposed on its surface. Alternatively, the AI surface protein may be isolated or purified from a bacterial culture of the non-pathogenic Gram positive bacteria. For example, the AI surface protein may be isolated from cell extracts or culture supernatants. Alternatively, the AI surface protein may be isolated or purified from the surface of the non-pathogenic Gram positive bacteria.

The non-pathogenic Gram positive bacteria may be used to express any of the Gram positive bacterial Adhesin Island proteins described herein, including proteins from a GBS Adhesin Island, a GAS Adhesin Island, or a S pneumo Adhesin Island. The non-pathogenic Gram positive bacteria are transformed to express an Adhesin Island surface protein. Preferably, the non-pathogenic Gram positive bacteria also express at least one Adhesin Island sortase. The AI transformed non-pathogenic Gram positive bacteria of the invention may be used to prevent or treat infection with a pathogenic Gram positive bacteria, such as GBS, GAS or Streptococcus pneumoniae. The non-pathogenic Gram positive bacteria may express the Gram positive bacterial Adhesin Island proteins in oligomeric forms that further comprise adhesin island proteins encoded within the genome of the non-pathogenic Gram positive bacteria.

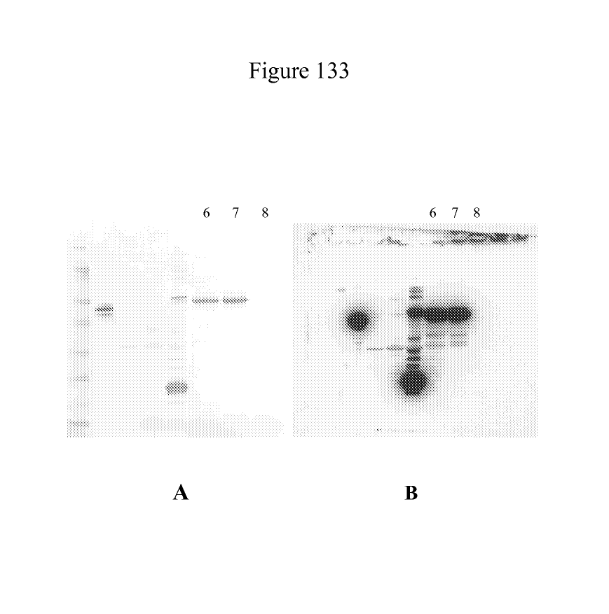

Applicants modified L. lactis to demonstrate that it can express GBS AI polypeptides. L. lactis was transformed with a construct encoding GBS 80 under its own promoter and terminator sequences. The transformed L. lactis appeared to express GBS 80 as shown by Western blot analysis using anti-GBS 80 antiserum. See lanes 6 and 7 of the Western Blots provided in FIGS. 133A and 133B (133A and 133B are two different exposures of the same Western blot). See also Example 13.

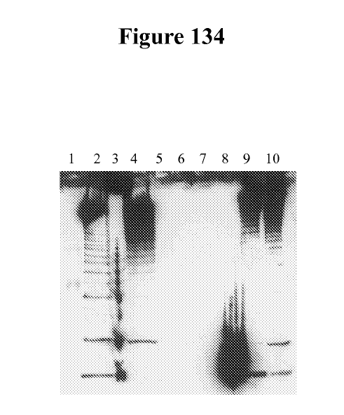

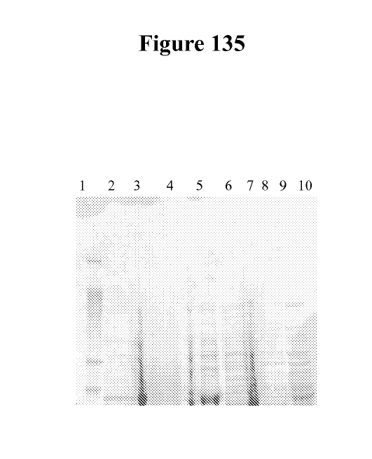

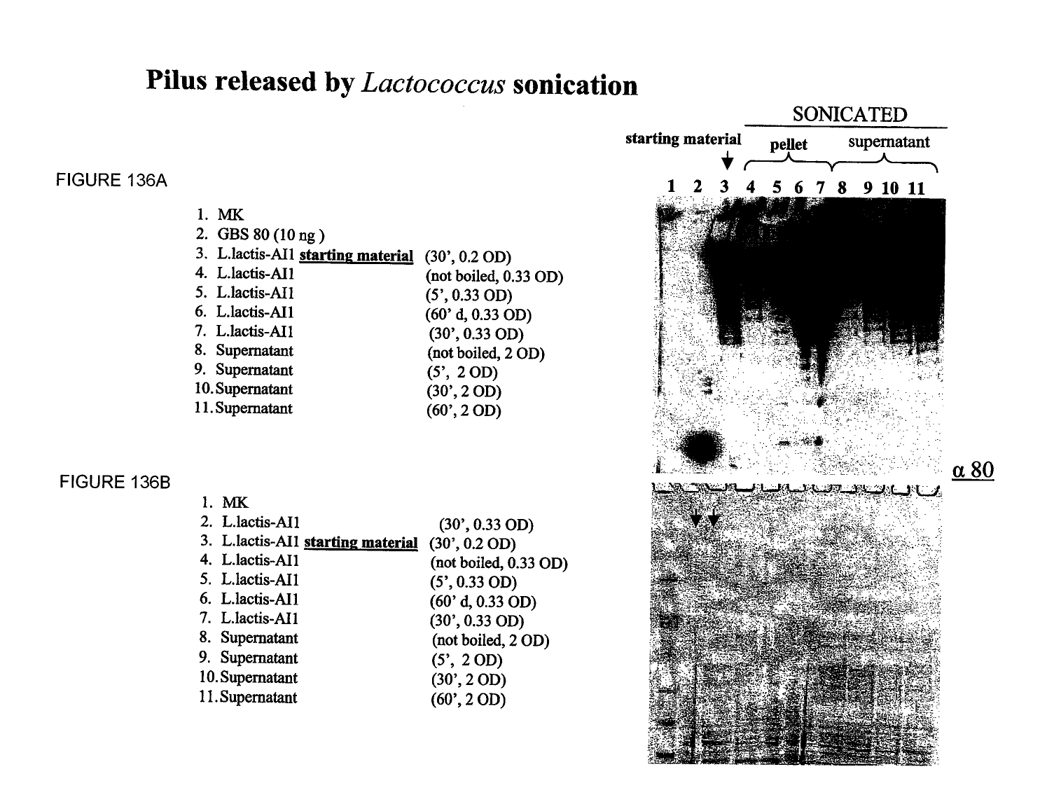

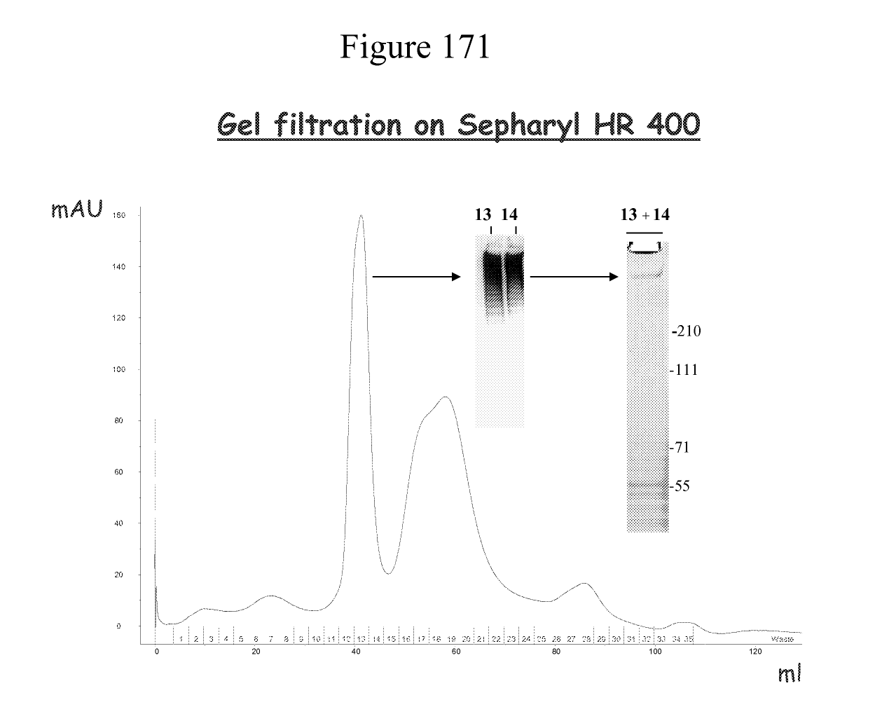

Applicants also transformed L. lactis with a construct encoding GBS AI-1 polypeptides GBS 80, GBS 52, SAG0647, SAG0648, and GBS 104 under the GBS 80 promoter and terminator sequences. These L. lactis expressed high molecular weight structures that were immunoreactive with anti-GBS 80 in immunoblots. See FIG. 134, lane 2, which shows detection of a GBS 80 monomer and higher molecular weight polymers in total transformed L. lactis extracts. Thus, it appeared that L. lactis is capable of expressing GBS 80 in oligomeric form. The high molecular weight polymers were not only detected in L. lactis extracts, but also in the culture supernatants. See FIG. 135 at lane 4. See also Example 14. Thus, the GBS AI polypeptides in oligomeric form can be isolated and purified from either L. lactis cell extracts or culture supernatants. These oligomeric forms can, for instance, be isolated from cell extracts or culture supernatants by release by sonication. See FIG. 136A and B. See also FIG. 171, which shows purification of GBS pili from whole extracts of L. lactis expressing the GBS AI-1 following sonication and gel filtration on a Sephacryl HR 400 column.

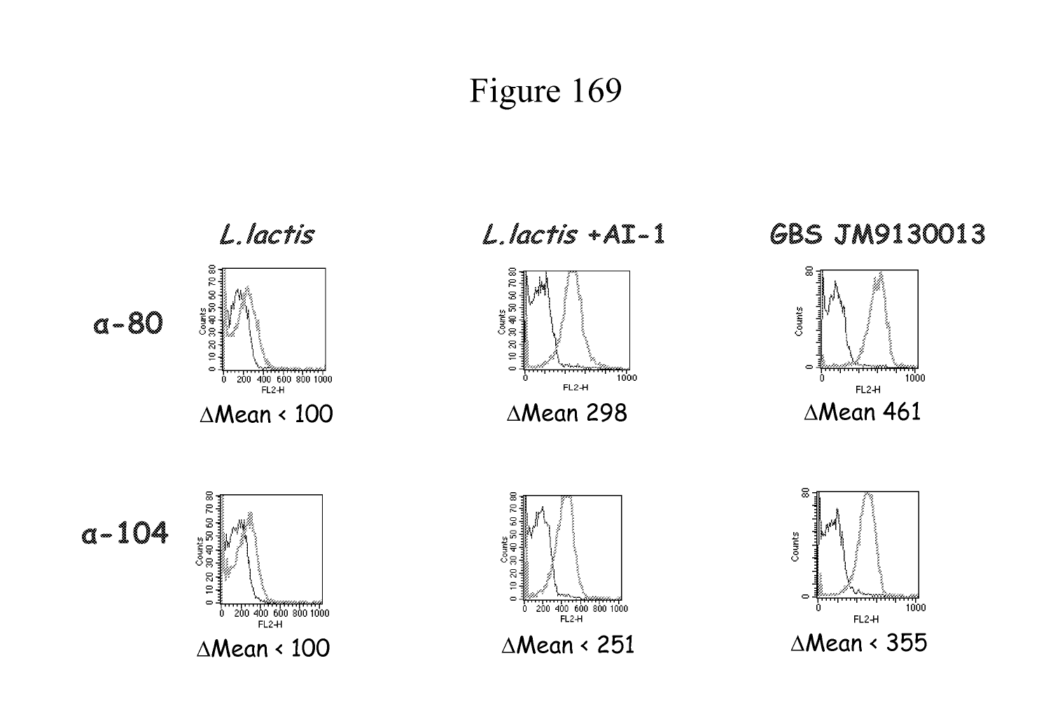

Furthermore, the L. lactis transformed with the construct encoding GBS AI-1 polypeptides GBS 80, GBS 52, SAG0647, SAG0648, and GBS 104 under the GBS 80 promoter and terminator sequences expressed the GBS AI-1 polypeptides on its surface. FACS analysis of these transformed L. lactis detected cell surface expression of both GBS 80 and GBS 104. The surface expression levels of GBS 80 and GBS 104 on the transformed L. lactis were similar to the surface expression levels of GBS 80 and GBS 104 on GBS strains COH1 and JM9130013, which naturally express GBS AI-1. See FIG. 169 for FACS analysis data for L. lactis transformed with GBS AI-1 and wildtype JM9130013 bacteria using anti-GBS 80 and GBS 104 antisera. Table 40 provides the results of FACS analysis of transformed L. lactis, COH1, and JM9130013 bacteria using anti-GBS 80 and anti-GBS 104 antisera. The numbers provided represent the mean fluorescence value difference calculated for immune versus pre-immune sera obtained for each bacterial strain.

TABLE 40
FACS analysis of L. lactis and GBS
bacteria strains expressing GBS AI-1

| | Anti-GBS 80 | Anti-GBS 104 |
|---|---|---|
| | antiserum | antiserum |
| GBS AI-1 transformed L. lactis | 298 | 251 |
| GBS COH1 | 305 | 305 |
| GBS JM9130013 | 461 | 355 |

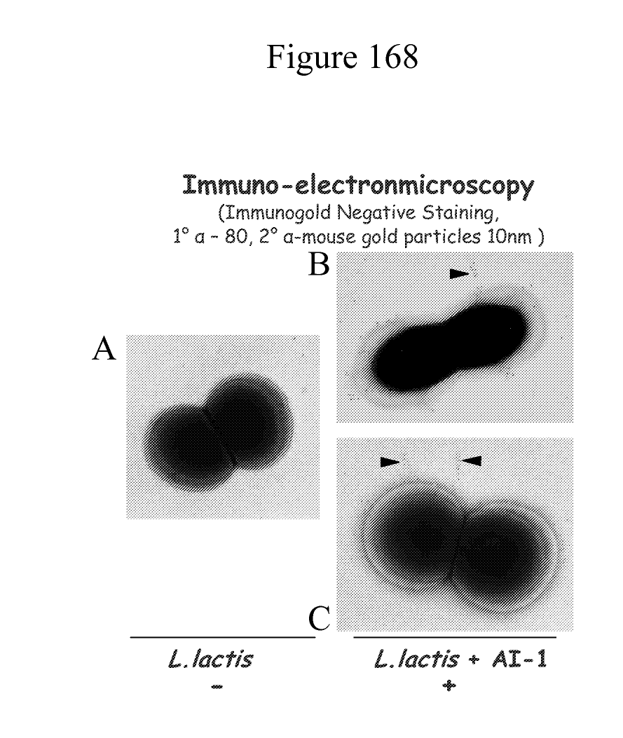

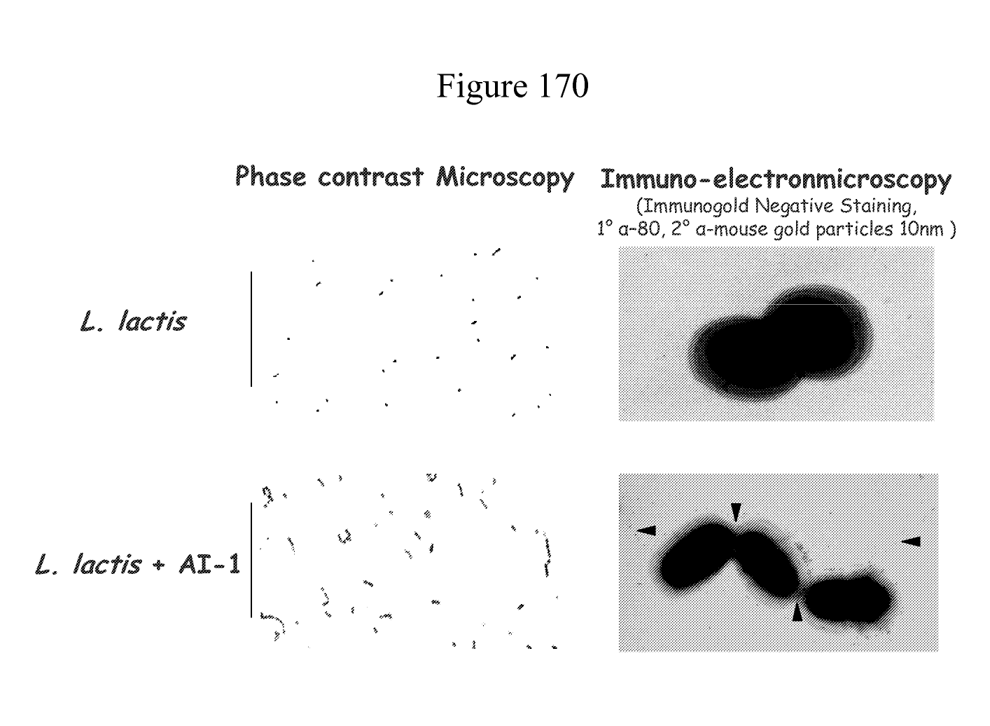

Immunogold-electronmicroscopy performed with anti-GBS 80 primary antibodies detected the presence of pilus structures on the surface of the L. lactis bacteria expressing GBS AI-1, confirming the results of the FACS analysis. See FIG. 168B and C. Interestingly, this expression of GBS pili on the surface of the L. lactis induced L. lactis aggregation. See FIG. 170. Thus, GBS AI polypeptides may also be isolated and purified from the surface of L. lactis. The ability of L. lactis to express GBS AI polypeptides on its surface also demonstrates that it may be useful as a host to deliver GBS AI antigens.

In fact, immunization of mice with L. lactis transformed with GBS AI-1 was protective in a subsequent challenge with GBS. Female mice were immunized with L. lactis transformed with GBS AI-1. The immunized female mice were bred and their pups were challenged with a dose of GBS sufficient to kill 90% of non-immunized pups. Detailed protocols for intranasal and subcutaneous immunization of mice with transformed L. lactis can be found in Examples 18 and 19, respectively. Table 43 provides data showing that immunization of the female mice with L. lactis expressing GBS AI-1 (LL-AI 1) greatly increased survival rate of challenged pups relative to both a negative PBS control (PBS) and a negative L. lactis control (LL 10 E9, which is wild type L. lactis not transformed to express GBS AI-1).

TABLE 43
Protection of Mice Immunized with L. lactis expressing GBS AI-1

| | | | | Survival | |
|---|---|---|---|---|---|
| Immunization | | Alive/ | Survival | % | |
| Route | Antigen | Treated | % | Range | p value |
| Intraperitoneum | Recombinant | 16/18 | 89 | 80-100 | <0.001 |
| | GBS 80 | | | | |
| Subcutaneous | LL-AI 1 10 E9 | 40/49 | 82 | 70-90 | <0.001 |
| | LL-AI 1 10 | 50/60 | 83 | 60-100 | <0.001 |
| | E10 | | | | |
| | PBS | 4/30 | 13 | 0-30 | |
| | LL 10 E9 | 3/57 | 5 | 0-20 | |
| Intranasal | LL-AI 1 10 E9 | 22/60 | 37 | 0-100 | 0.02 |
| | LL-AI 1 10 | 31/49 | 63 | 30-90 | <0.001 |
| | E10 | | | | |
| | LL 10 E9 | 2/27 | 7 | 0-20 | |

Table 51 provides further evidence that immunization of mice with L. lactis transformed with GBS AI-1 is protective against GBS.

TABLE 51
Further Protection of Mice Immunized with
L. lactis expressing GBS AI-1

| | Immunization | Alive/ | Survival % |
|---|---|---|---|
| Antigen | route | Treated | (p < 0.0000001) |
| recombinant GBS 80 | IP | 48/50 | 92 |
| Recombinant GBS 80 | SC | 21/30 | 70 |
| L. lactis + AI1 10⁶ cfu | SC | 6/66 | 9 |
| L. lactis + AI1 10⁷ cfu | SC | 47/70 | 73 |
| L. lactis + AI1 10⁸ cfu | SC | 116/153 | 76 |
| L. lactis + AI1 10⁹ cfu | SC | 98/118 | 83 |
| L. lactis + AI1 10¹⁰ cfu | SC | 107/129 | 83 |
| L. lactis 10¹⁰ cfu | SC | 4/83 | 5 |
| PBS | SC | 6/110 | 5 |
| L. lactis + AI1 10¹⁰ cfu | IN | 51/97 | 52 |
| L. lactis 10¹¹ cfu | IN | 1/40 | 7 |
| PBS | IN | 0/37 | 0 |

Protection of immunized mice with L. lactis expressing the GBS AI-1 is at least partly due to a newly raised antibody response. Table 46 provides anti-GBS 80 antibody titers detected in serum of the mice immunized with L. lactis expressing the GBS AI-1 as described above. Mice immunized with L. lactis expressing the GBS AI-1 have anti-GBS 80 antibody titres, which are not observed in mice immunized with L. lactis not transformed to express the GBS AI-1. Further, as expected from the survival data, mice subcutaneously immunized with L. lactis transformed to express the GBS AI-1 have significantly higher serum anti-GBS 80 antibody titers than mice intranasally immunized with L. lactis transformed to express the GBS AI-1.

TABLE 46
Antibody Responses against GBS 80 in Serum of Mice
Immunized with L. lactis Expressing GBS AI-1

| | Ab Titre Obtained Following | | |
|---|---|---|---|
| | Subcutaneous | Intranasal | Intraperitoneal |
| Antigen | Immunization | Immunization | Immunization |
| LL 10 E9 | 0 | 0 | |
| LL-AI 1 10 E9 | 14000 | 50 | |
| LL-AI 1 10 E10 | 25000 | 406 | |
| Recombinant GBS 80 | | | 120000 |

Anti-GBS 80 antibodies of the IgA isotype were specifically detected in various body fluids of the mice subcutaneously or intranasally immunized with L. lactis expressing the GBS AI-1.

TABLE 47
Anti-GBS 80 IgA Antibodies Detected in Mouse Tissues Following
Immunization with L. lactis Expressing GBS AI-1

| | Immunization | Anti-GBS 80 IgA Antibodies Detected in | | |
|---|---|---|---|---|
| Antigen | route | Serum | Vaginal Wash | Nasal Wash |
| LL 10 E9 | | 0 | 0 | 0 |
| LL-AI 1 | Subcutaneous | 0 | 25 | 20 |
| LL-AI 1 | Intranasal | 140 | 0 | 150 |
| GBS 80 | Intraperitoneal | 60 | 0 | |

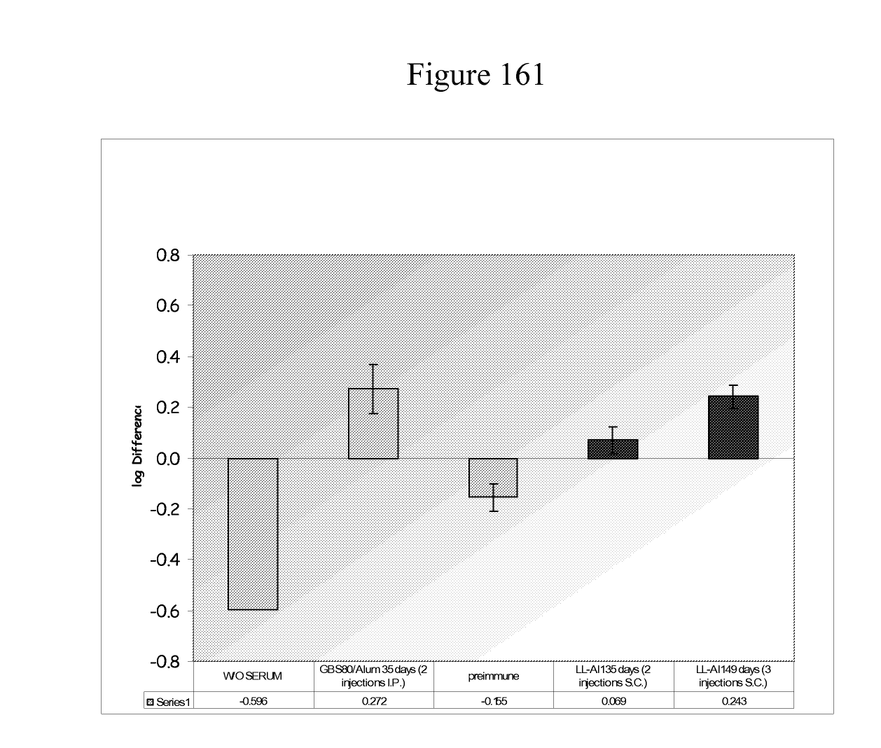

Furthermore, opsonophagocytosis assays also demonstrated that at least some of the antiserum produced against the L. lactis expressing GBS AI 1 is opsonic for GBS. See FIG. 161.

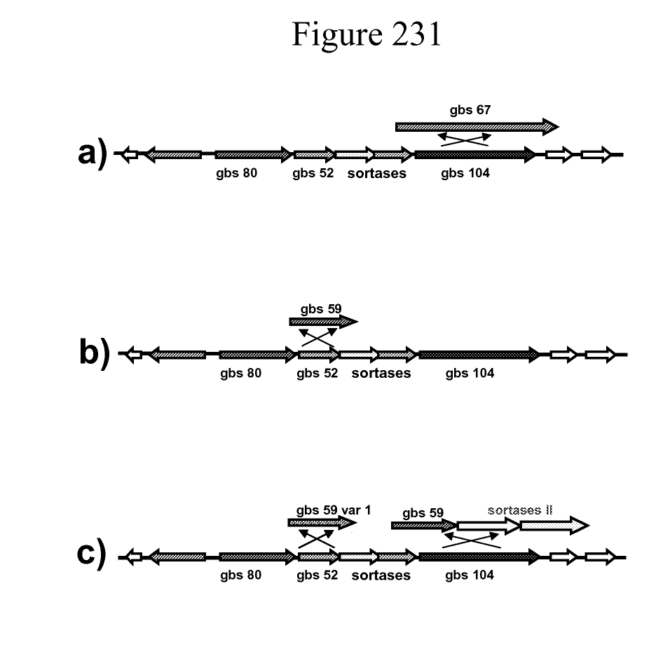

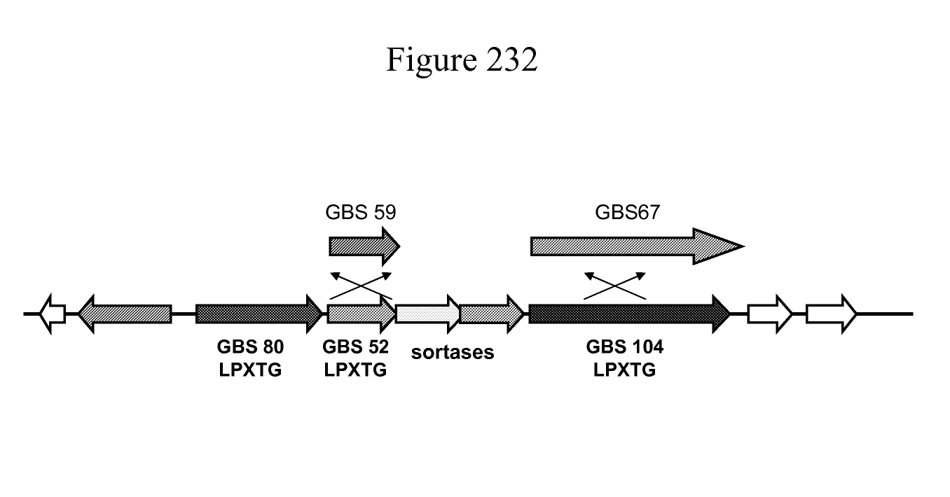

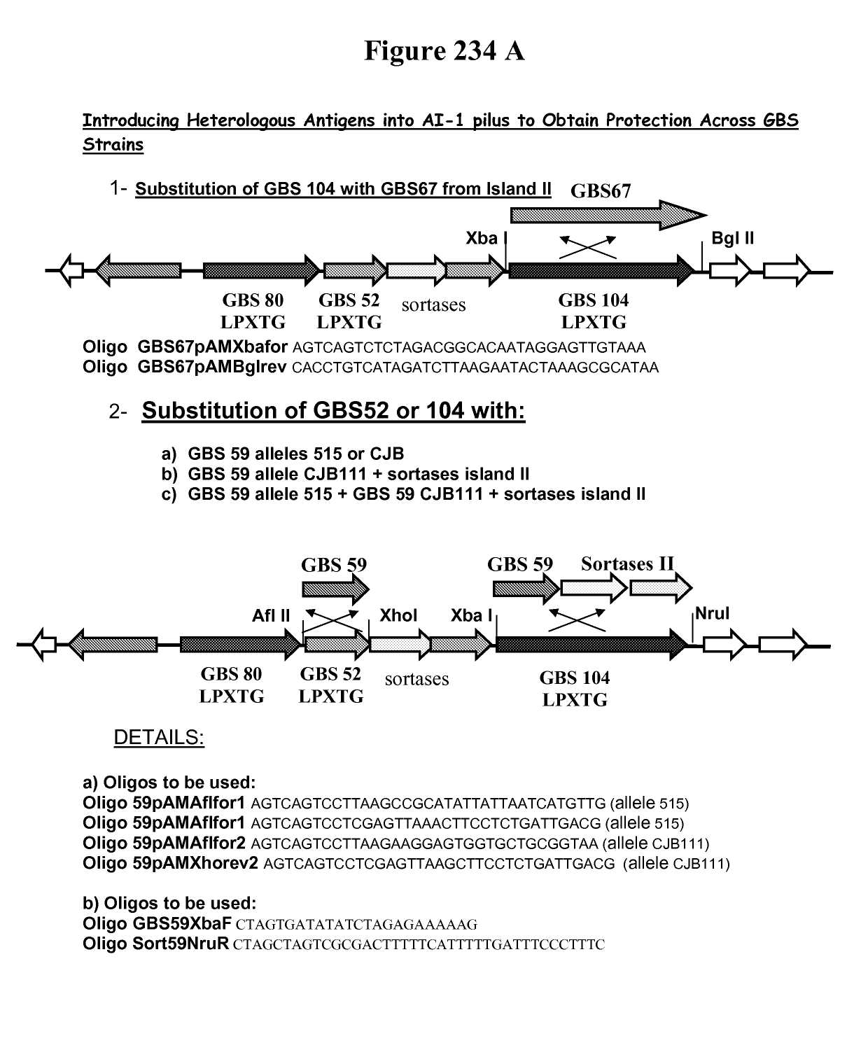

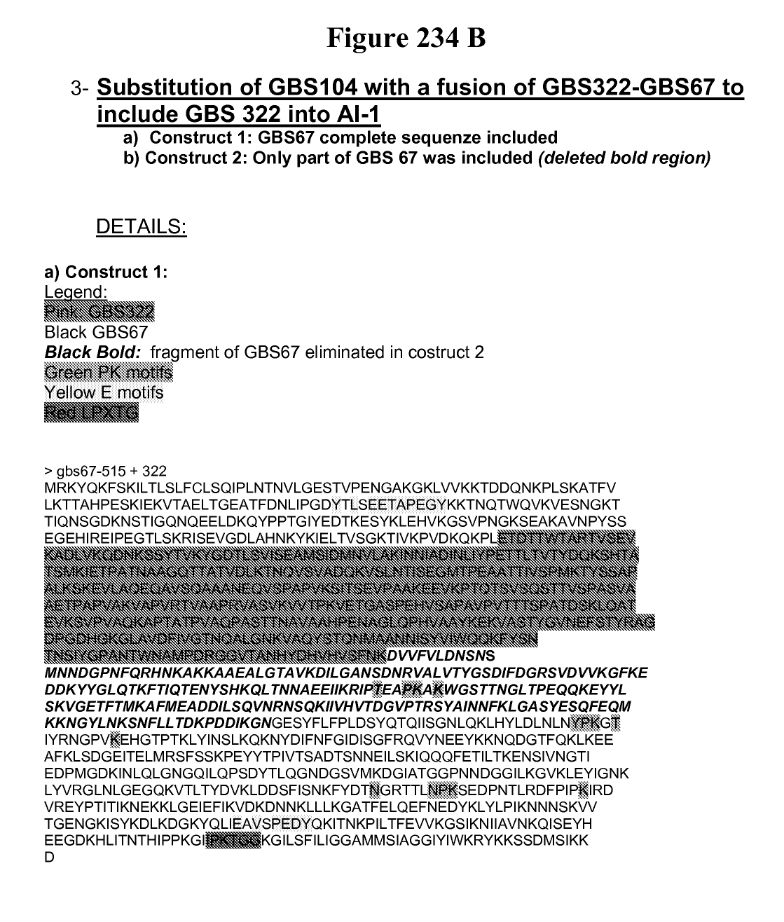

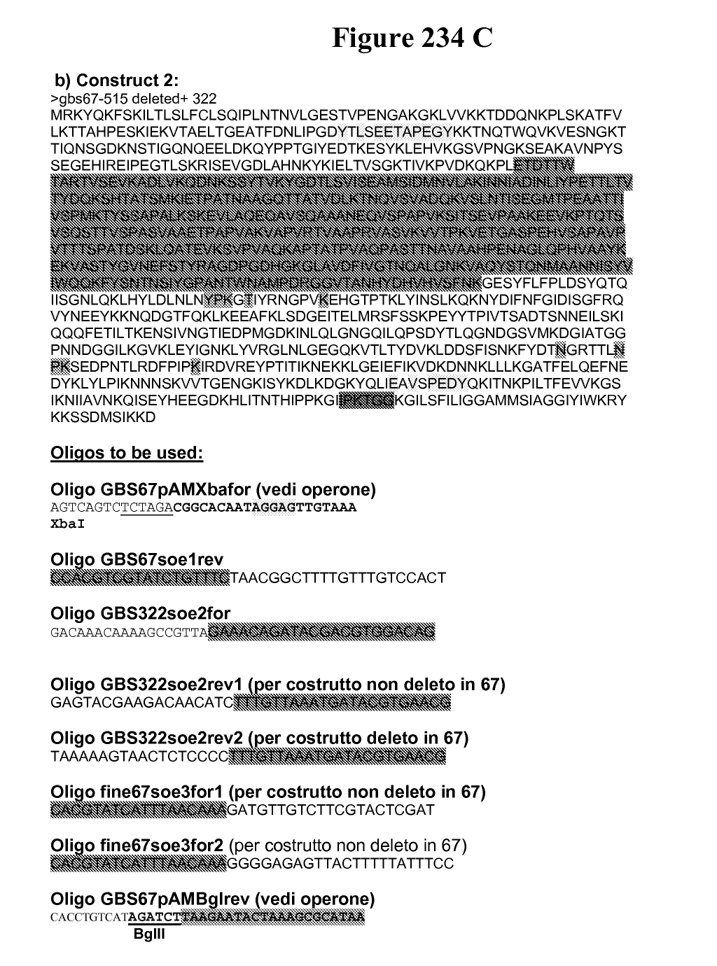

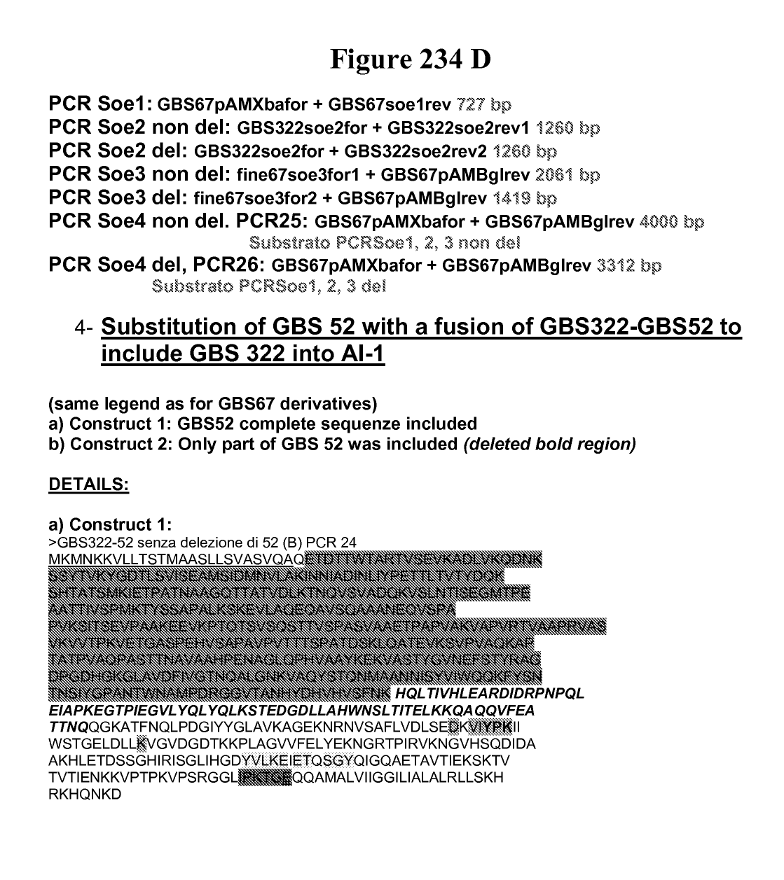

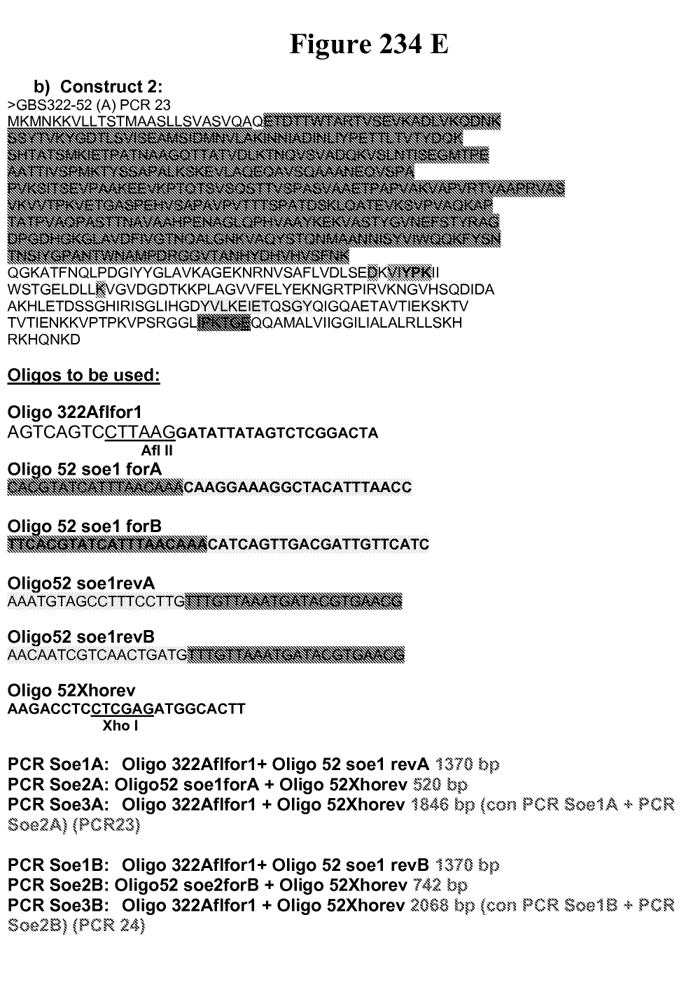

To obtain protection of against GBS across a greater number of strains and serotypes, it is possible to transform L. lactis with a recombinant GBS AI encoding both GBS AI-1 and AI-2, i.e., a hybrid GBS AI. By way of example, a hybrid GBS AI may be a GBS AI-1 with a replacement of the GBS 104 gene with a GBS 67 gene. A schematic of such a hybrid GBS AI is depicted in FIG. 231A. A hybrid GBS AI may alternatively be a GBS AI-1 with a replacement of the GBS 52 gene with a GBS 59 gene. See the schematic at FIG. 231B. Alternatively, a hybrid GBS AI may be a GBS AI-1 with a substitution of a GBS 59 polypeptide for the GBS 52 gene and a substitution of the GBS 104 gene for genes encoding GBS 59 and the two GBS AI-2 sortases. Another example of a hybrid GBS AI is a GBS AI-1 with the substitution of a GBS 59 gene for the GBS 52 gene and a GBS 67 for the GBS 104 gene. See the schematic at FIG. 232. A further example of a hybrid GBS AI is a GBS AI-1 having a GBS 59 gene and genes encoding the GBS AI-2 sortases in place of the GBS 52 gene. Yet another example of a hybrid GBS AI is a GBS AI-1 with a substitution of either GBS 52 or GBS 104 with a fusion protein comprising GBS 322 and one of GBS 59, GBS 67, or GBS 150. Some of these hybrid GBS AIs may be prepared as briefly outlined in FIG. 234A-F.

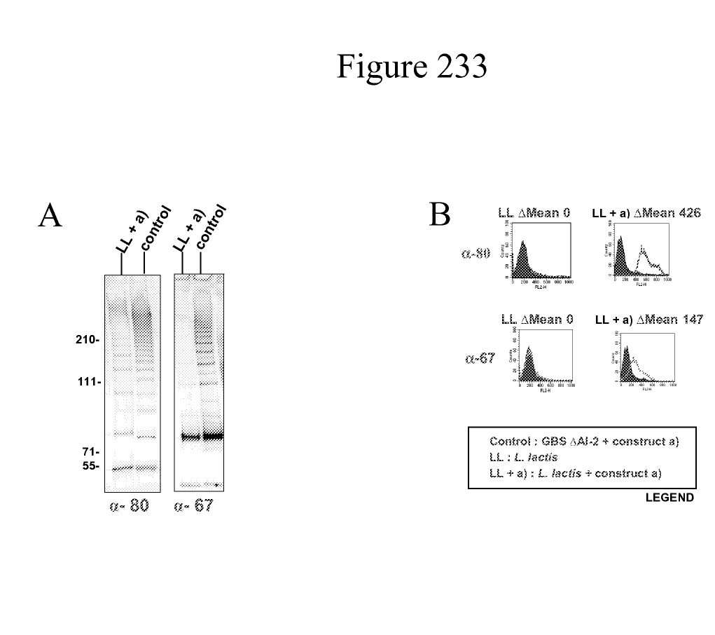

Applicants have prepared a hybrid GBS AI having a GBS AI-1 sequence with a substitution of a GBS 67 coding sequence for the GBS 104 gene as depicted in FIG. 231A. Transformation of L. lactis with the hybrid GBS AI-1 resulted in L. lactis expression of high molecular weight polymers containing the GBS 80 and GBS 67 proteins. See FIG. 233A, which provides Western blot analysis of L. lactis transformed with the hybrid GBS AI depicted in FIG. 231A. When L. lactis transformed with the hybrid GBS AI were probed with antibodies to GBS 80 or GBS 67, high molecular weight structures were detected. See lanes labelled LL+a) in both the α-80 and α-67 immunoblots. The GBS 80 and GBS 67 proteins were confirmed to be present on the surface of L. lactis by FACS analysis. See FIG. 233B, which shows a shift in fluorescence when GBS 80 and GBS 67 antibodies are used to detect GBS 80 and GBS 67 surface expression. The same shifts in fluorescence were not observed in L. lactis control cells, cells not transformed with the hybrid GBS AI.

Alternatively, the oligomeric, pilus-like structures may be produced recombinantly. If produced in a recombinant host cell system, the AI surface protein will preferably be expressed in coordination with the expression of one or more of the AI sortases of the invention. Such AI sortases will facilitate oligomeric or hyperoligomeric formation of the AI surface protein subunits.

AI Sortases of the invention will typically have a signal peptide sequence within the first 70 amino acid residues. They may also include a transmembrane sequence within 50 amino acid residues of the C terminus. The sortases may also include at least one basic amino acid residue within the last 8 amino acids. Preferably, the sortases have one or more active site residues, such as a catalytic cysteine and histidine.

As shown in FIG. 1, AI-1 includes the surface exposed proteins of GBS 80, GBS 52 and GBS 104 and the sortases SAG0647 and SAG0648. AI-1 typically appears as an insertion into the 3′ end of the trmA gene.

In addition to the open reading frames encoding the AI-1 proteins, AI-1 may also include a divergently transcribed transcriptional regulator such as araC (i.e., the transcriptional regulator is located near or adjacent to the AI protein open reading frames, but it transcribed in the opposite direction). It is believed that araC may regulate the expression of the AI operon. (See Korbel et al., Nature Biotechnology (2004) 22(7): 911-917 for a discussion of divergently transcribed regulators in E. coli).

AI-1 may also include a sequence encoding a rho independent transcriptional terminator (see hairpin structure in FIG. 1). The presence of this structure within the adhesin island is thought to interrupt transcription after the GBS 80 open reading frame, leading to increased expression of this surface protein.

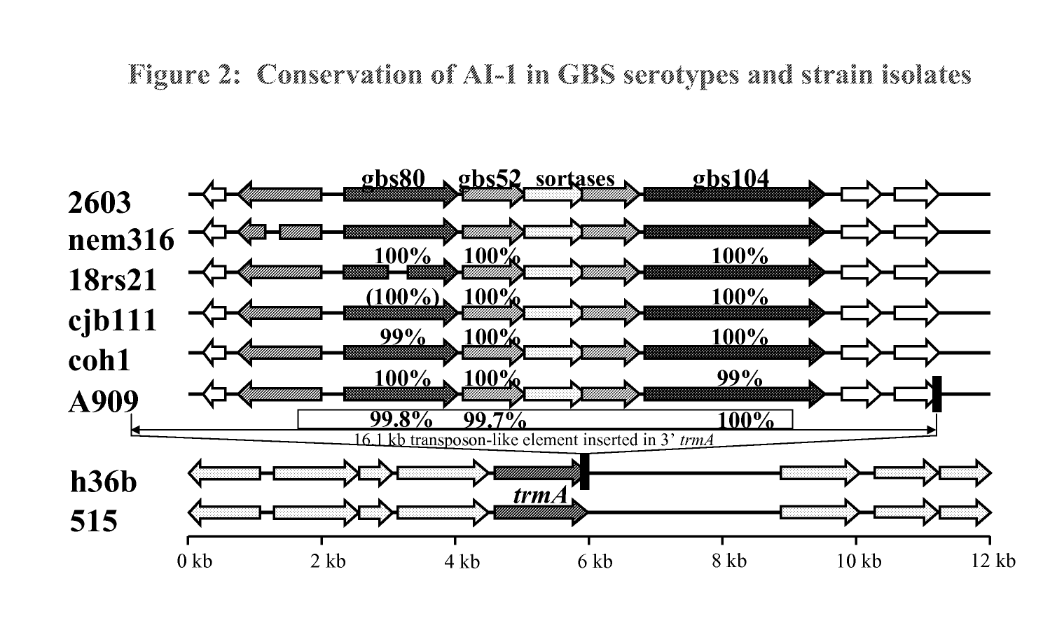

A schematic identifying AI-1 within several GBS serotypes is depicted in FIG. 2. AI-1 sequences were identified in GBS serotype V, strain isolate 2603; GBS serotype III, strain isolate NEM316; GBS serotype II, strain isolate 18RS21; GBS serotype V, strain isolate CJB111; GBS serotype III, strain isolate COH1 and GBS serotype 1a, strain isolate A909. (Percentages shown are amino acid identity to the 2603 sequence). (An AI-1 was not identified in GBS serotype 1b, strain isolate H36B or GBS serotype 1a, strain isolate 515).

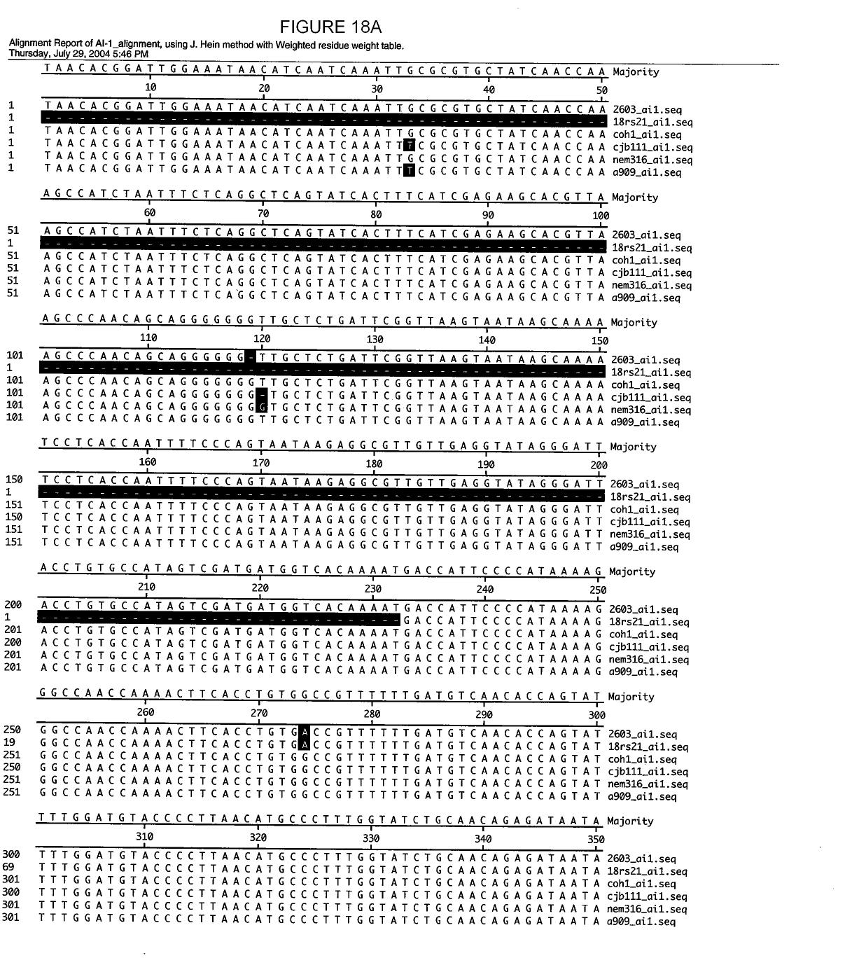

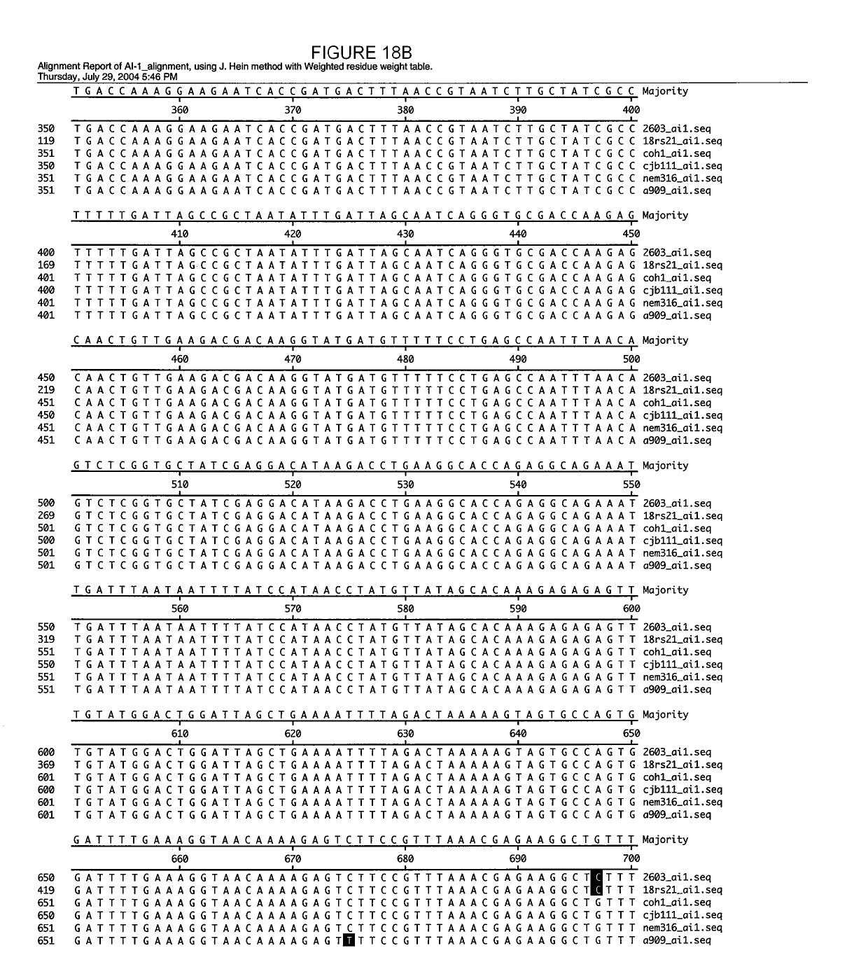

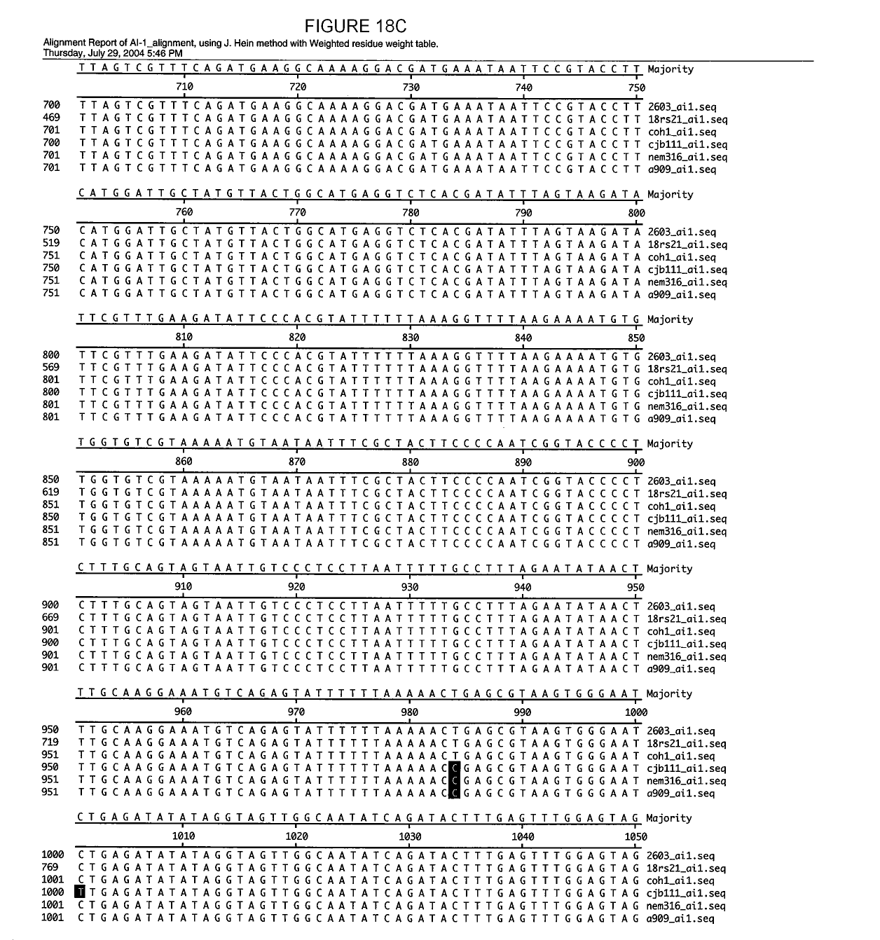

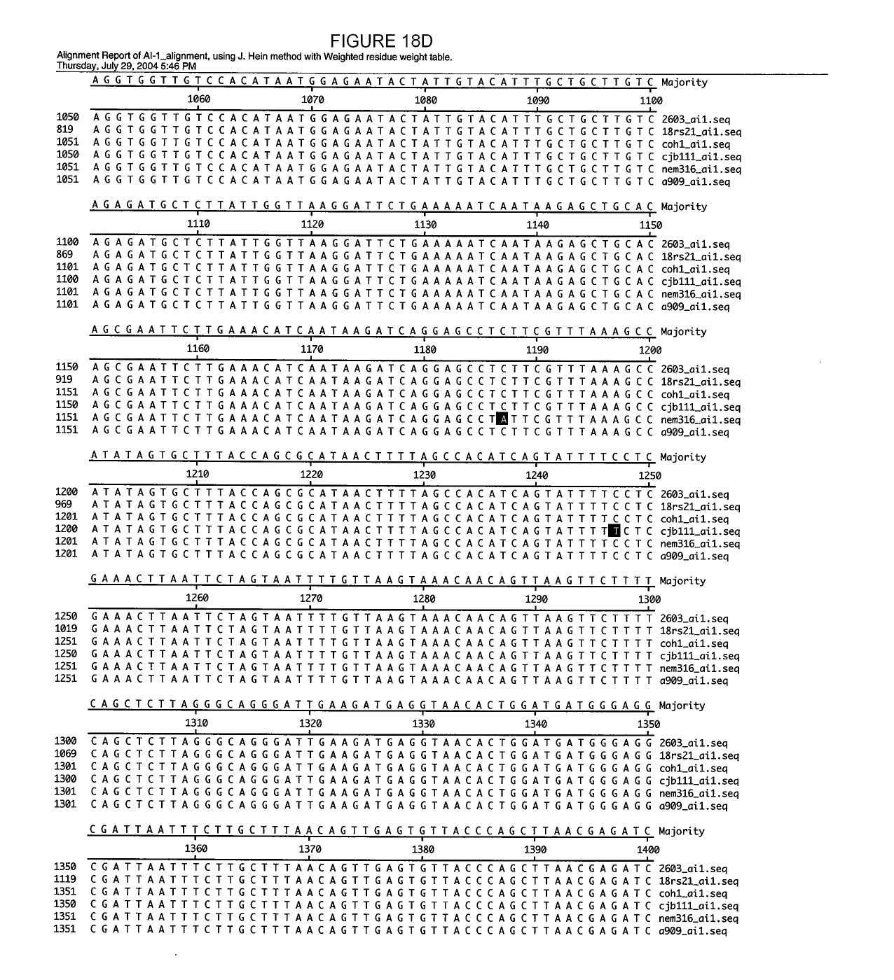

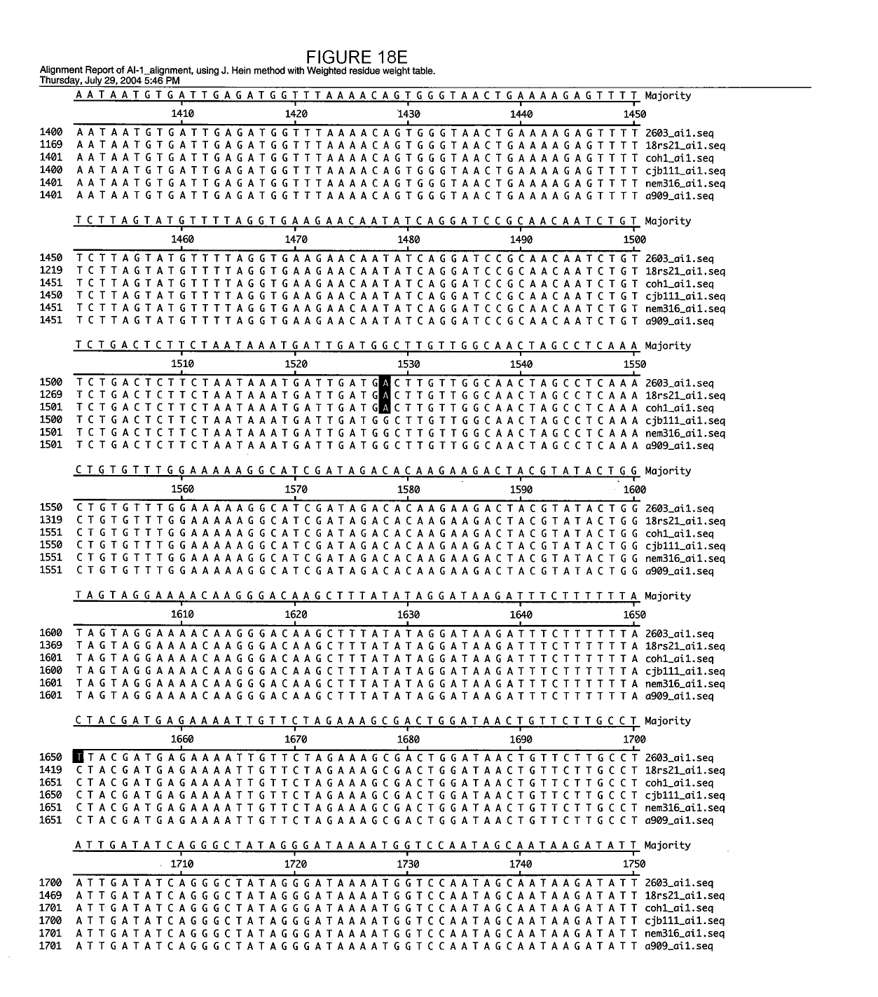

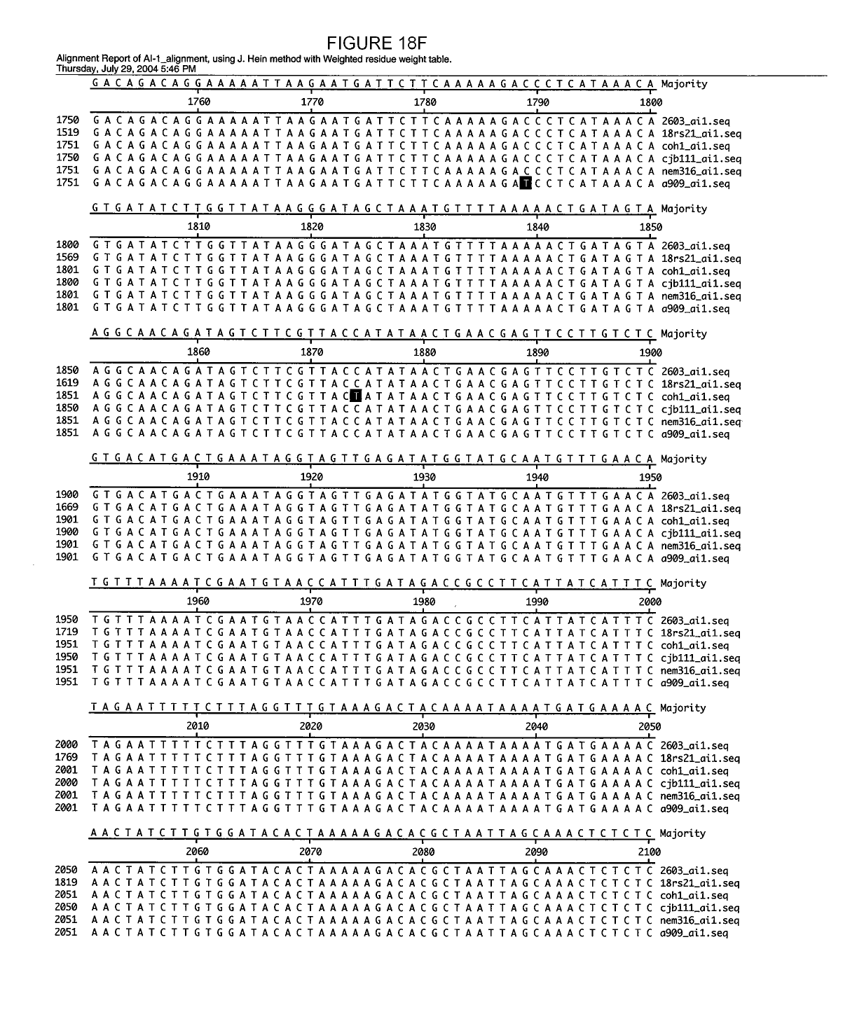

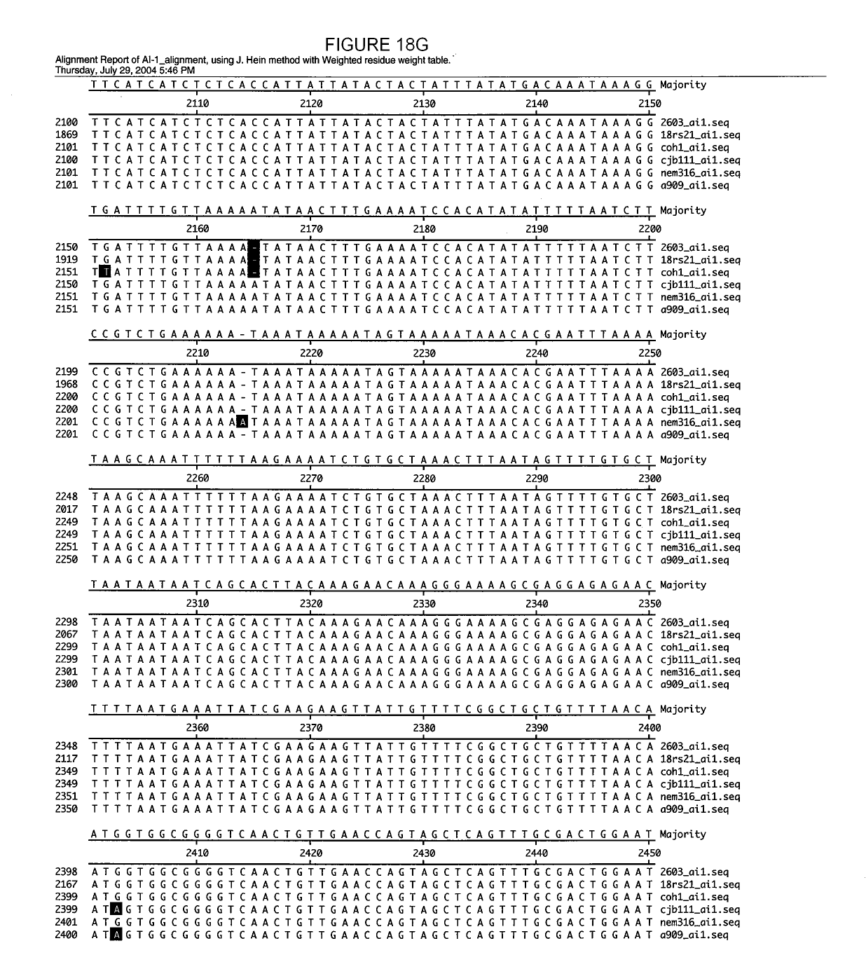

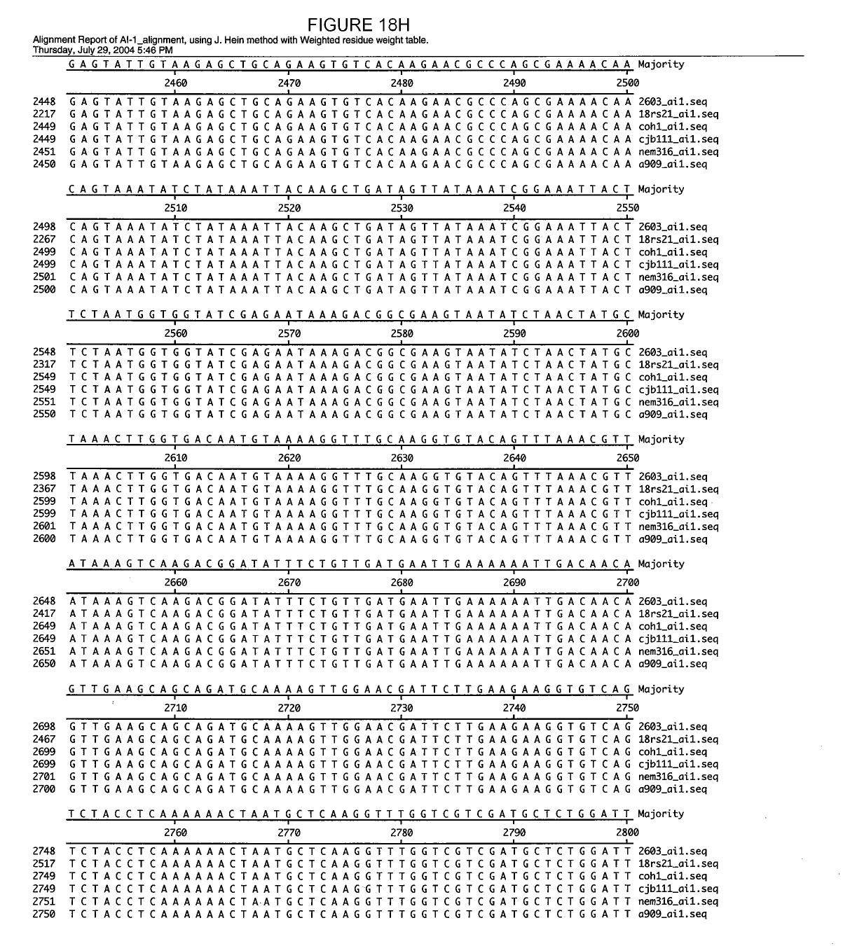

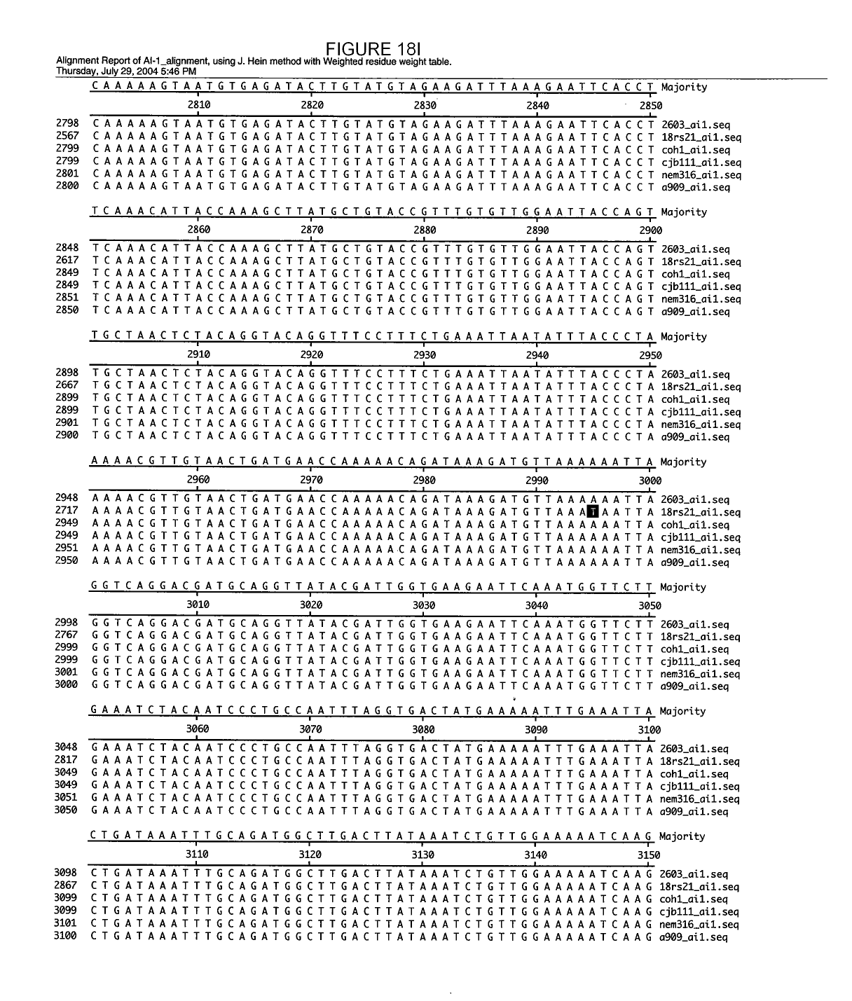

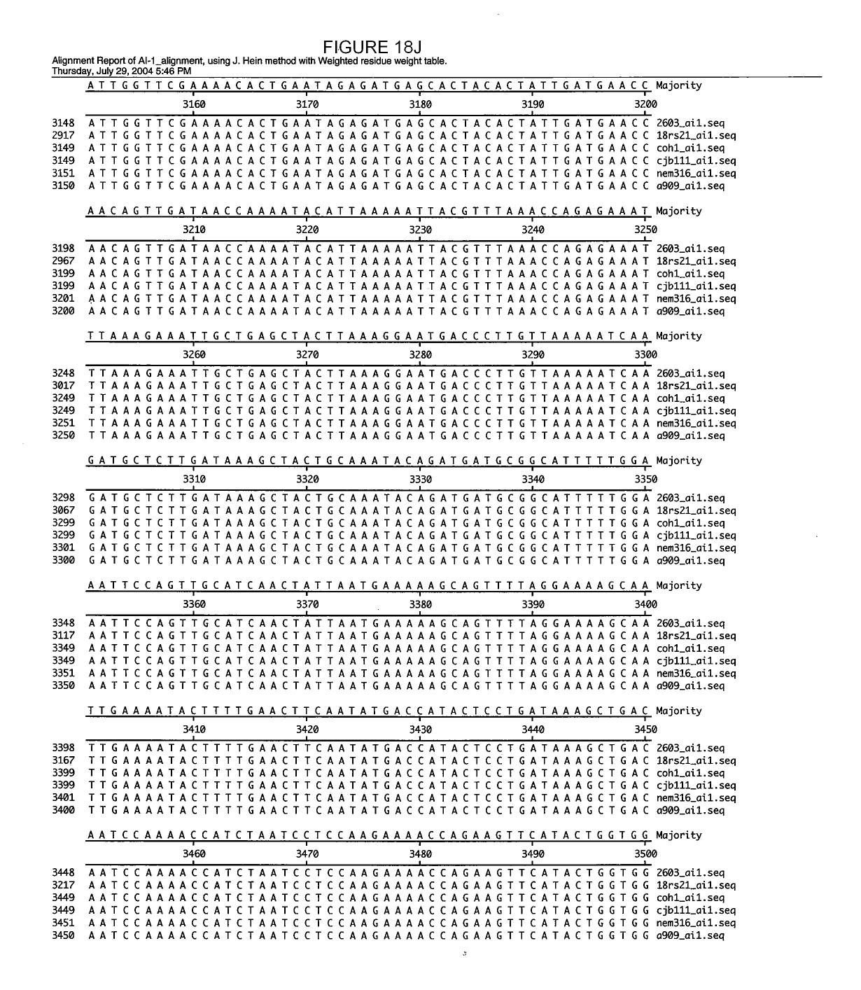

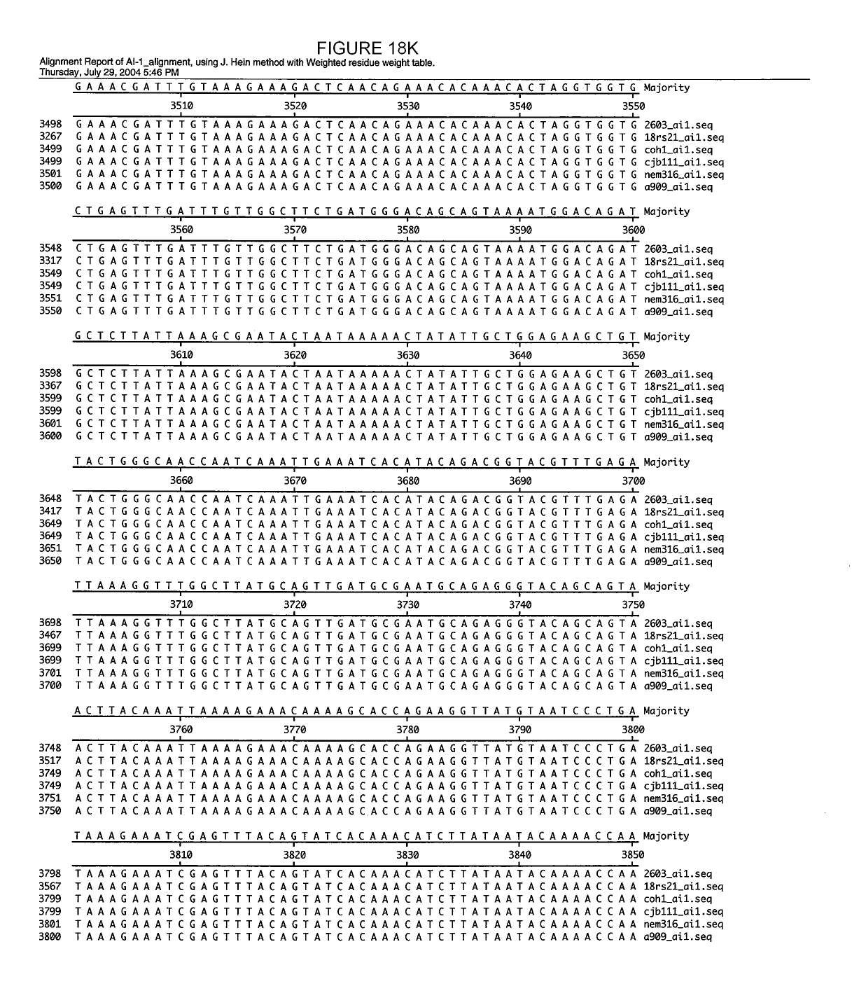

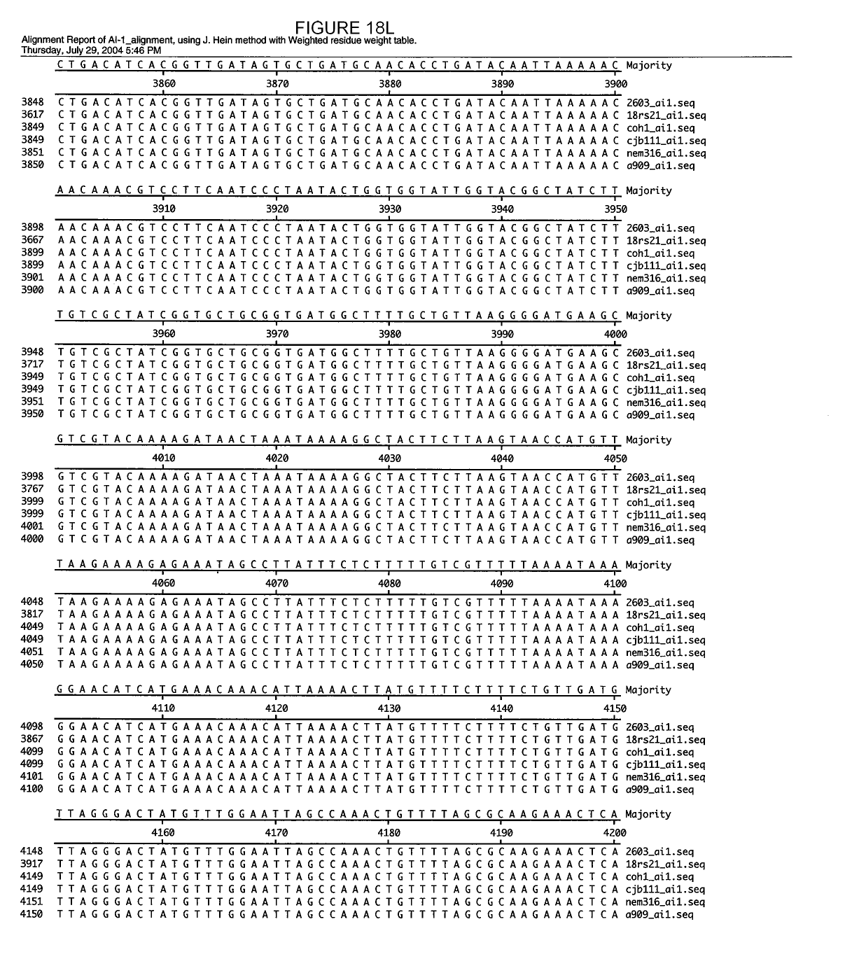

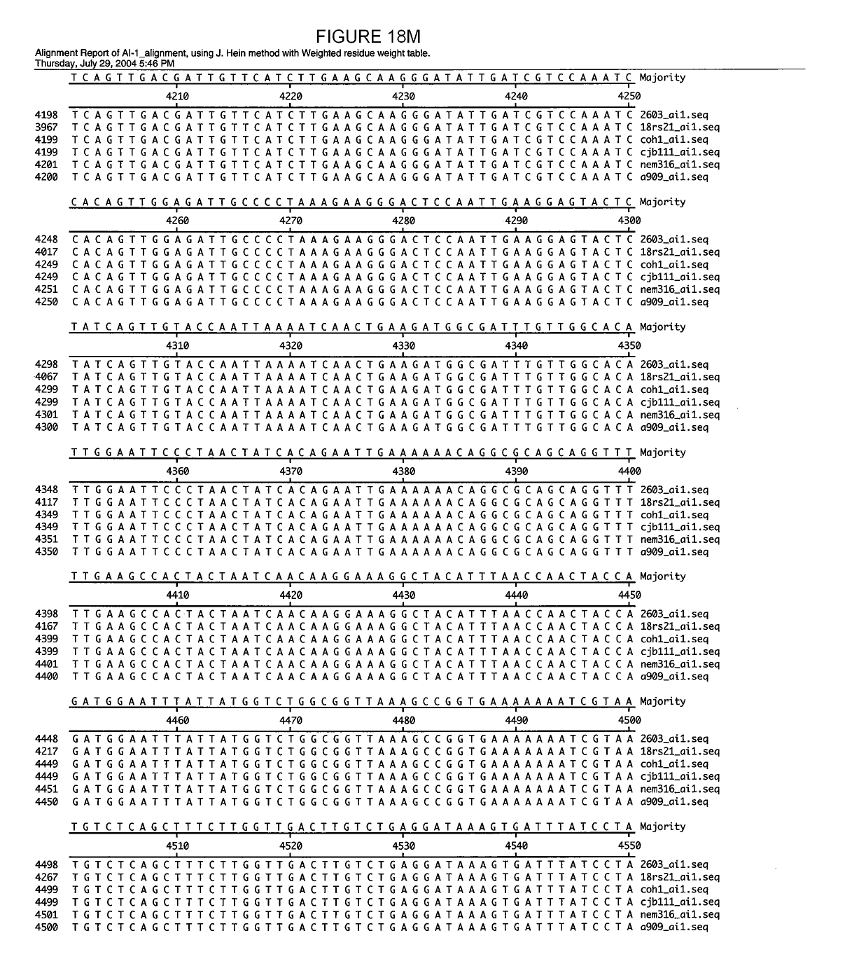

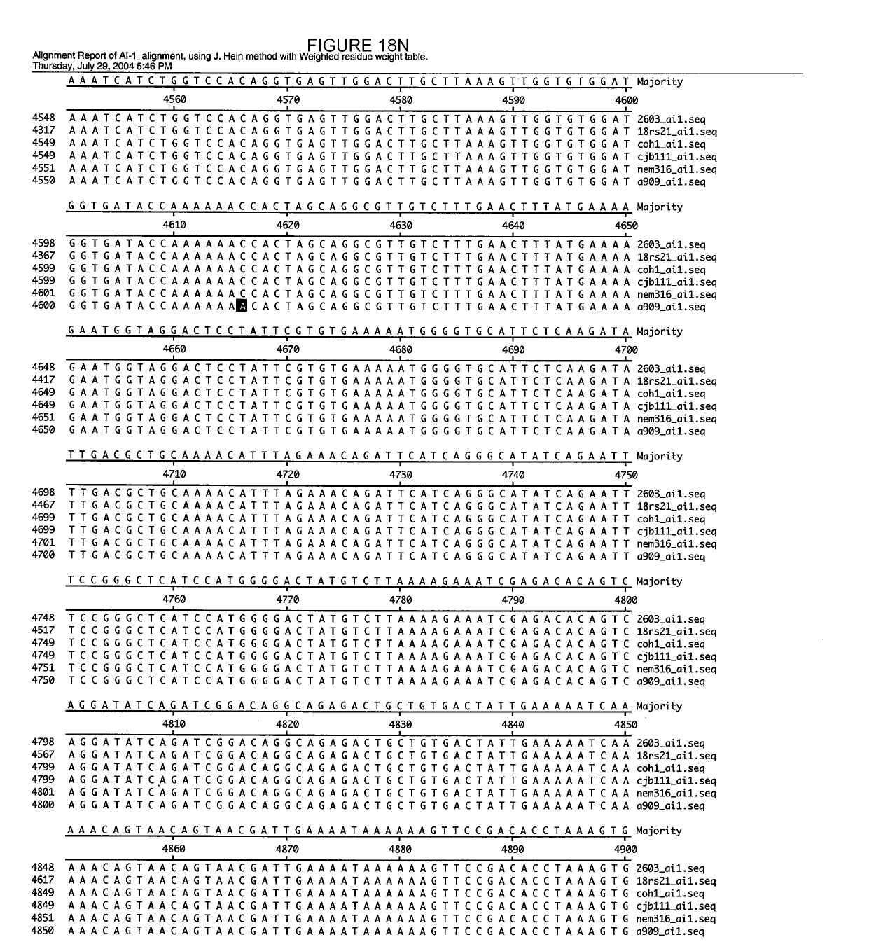

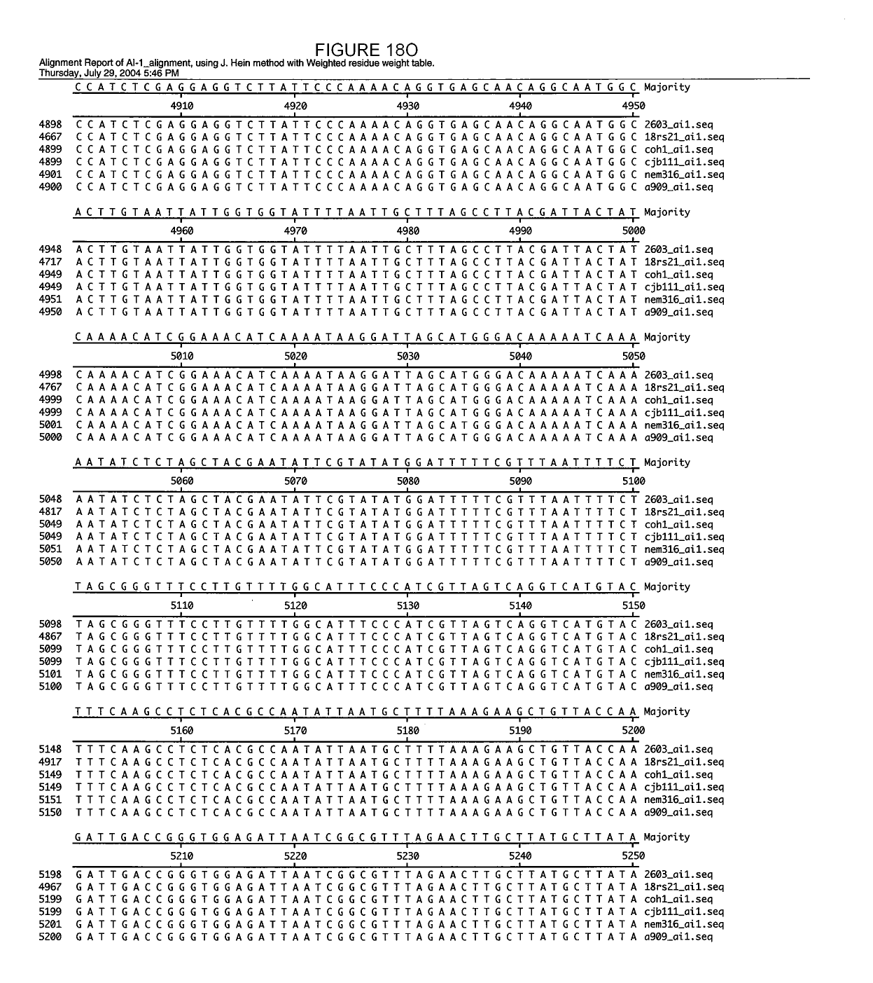

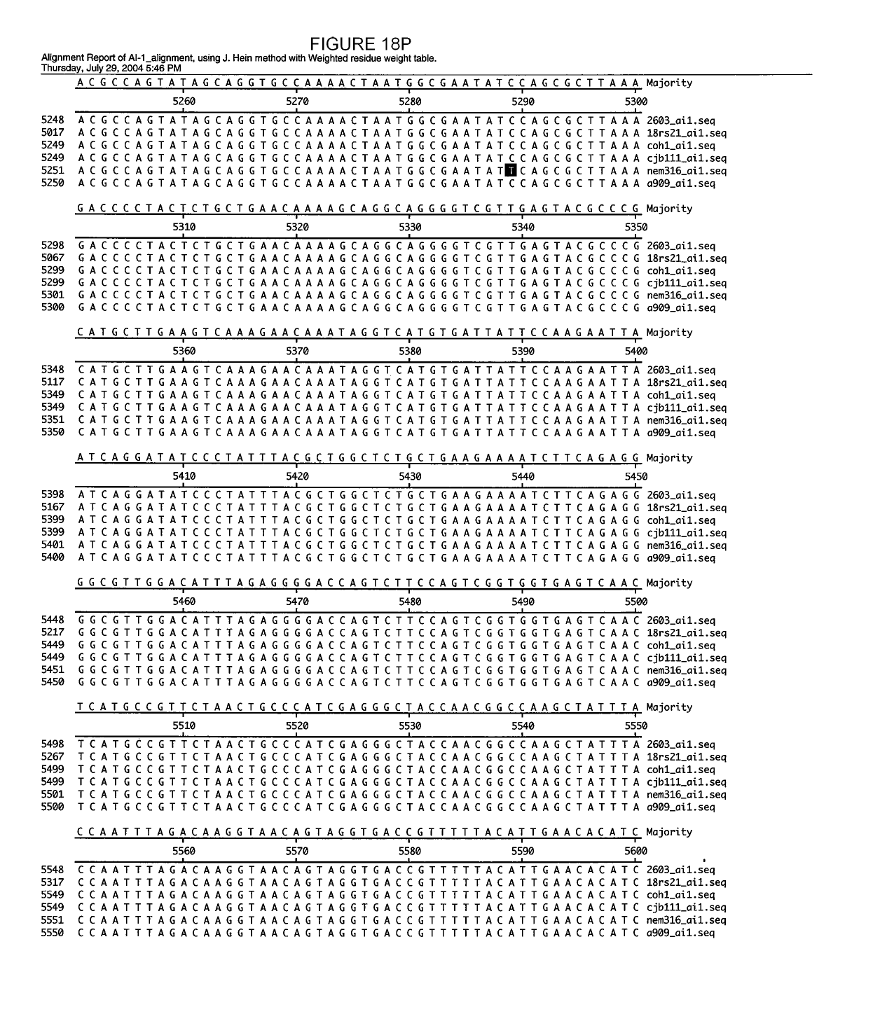

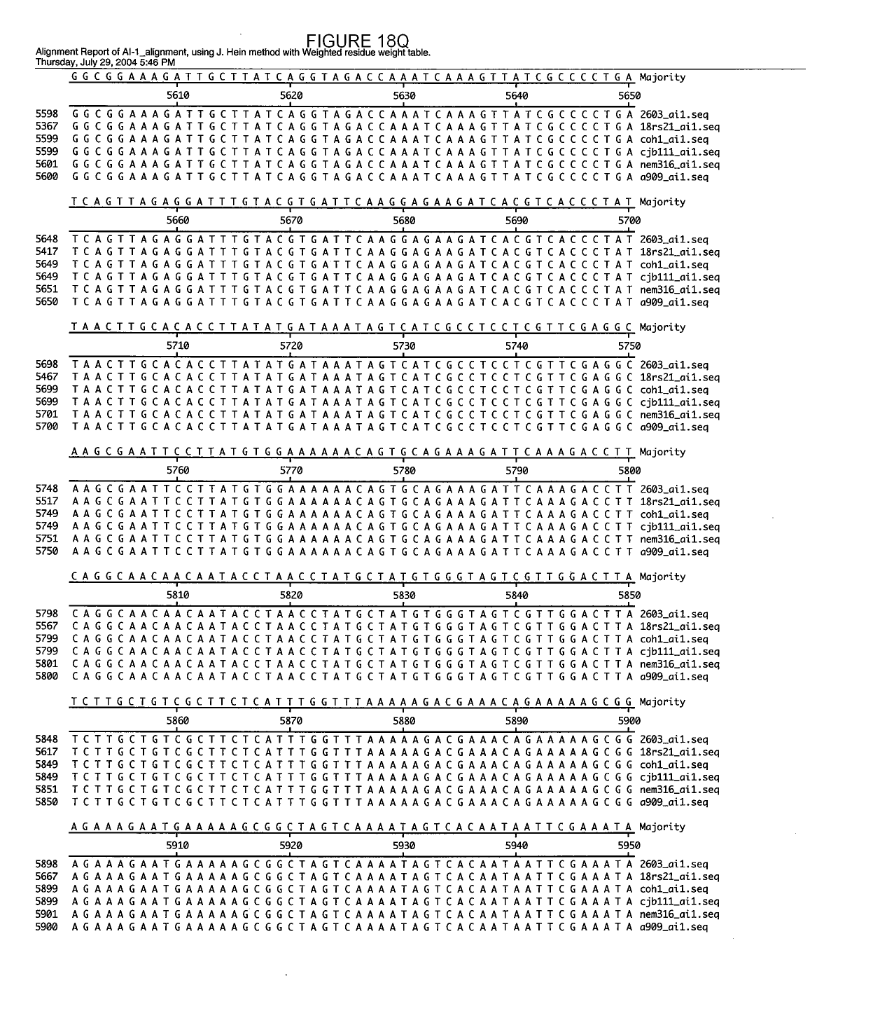

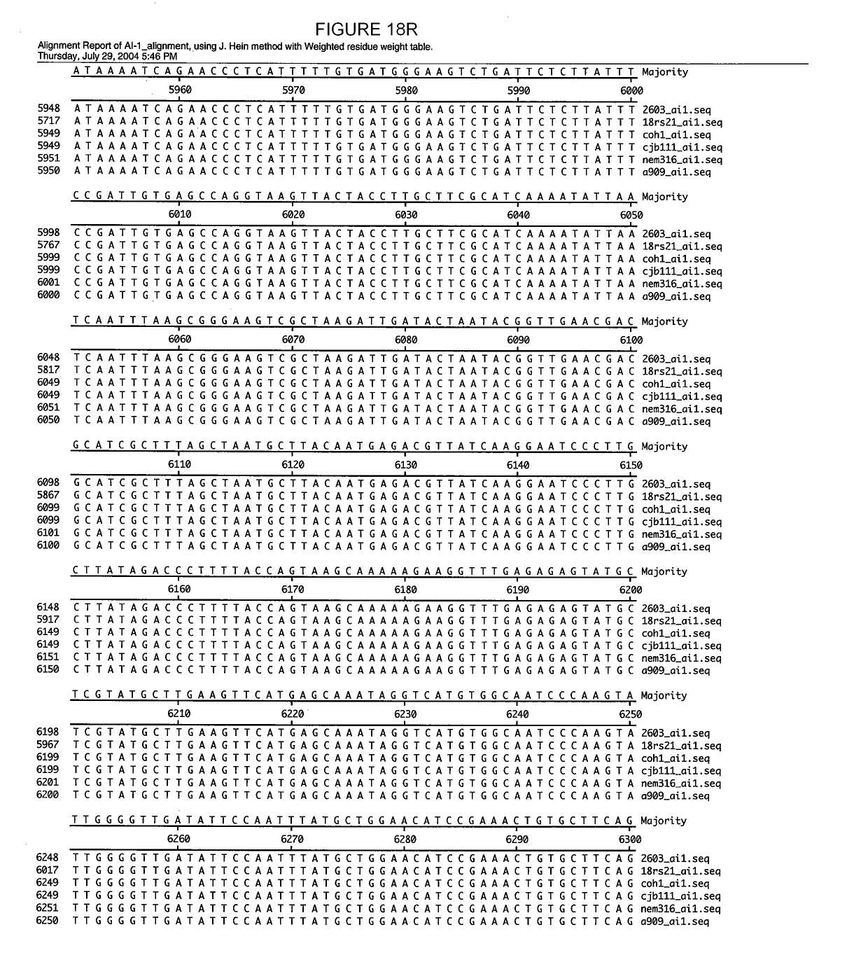

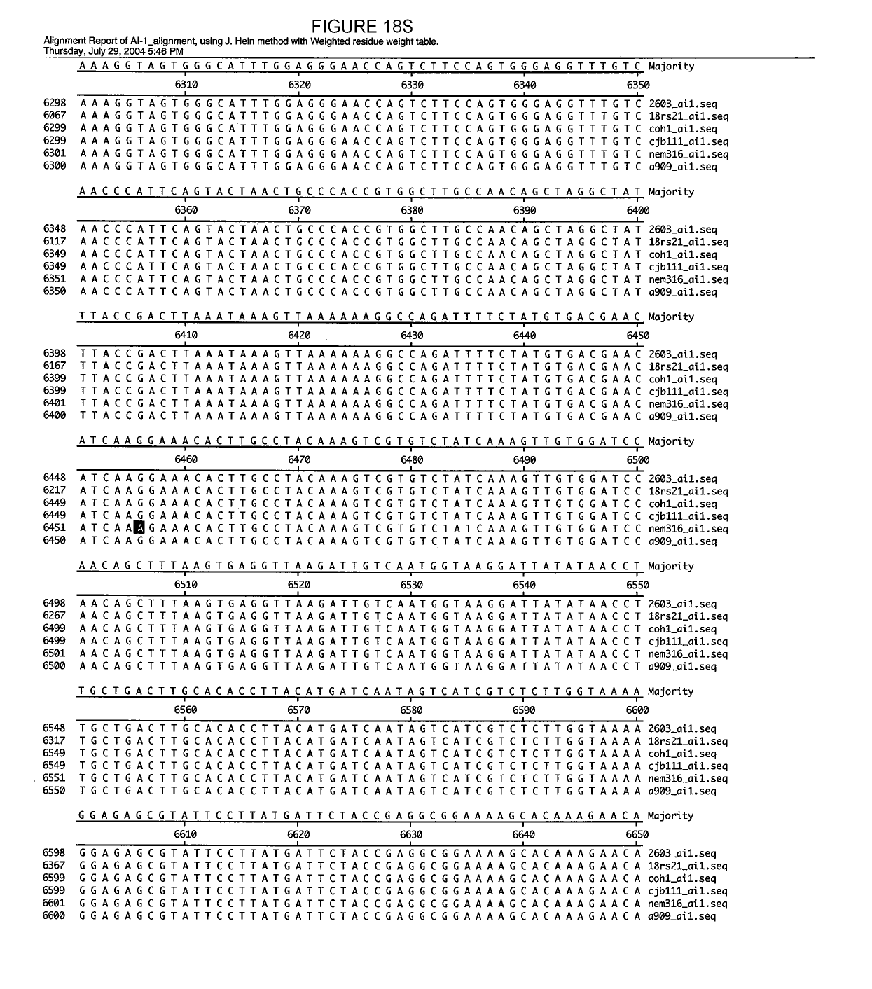

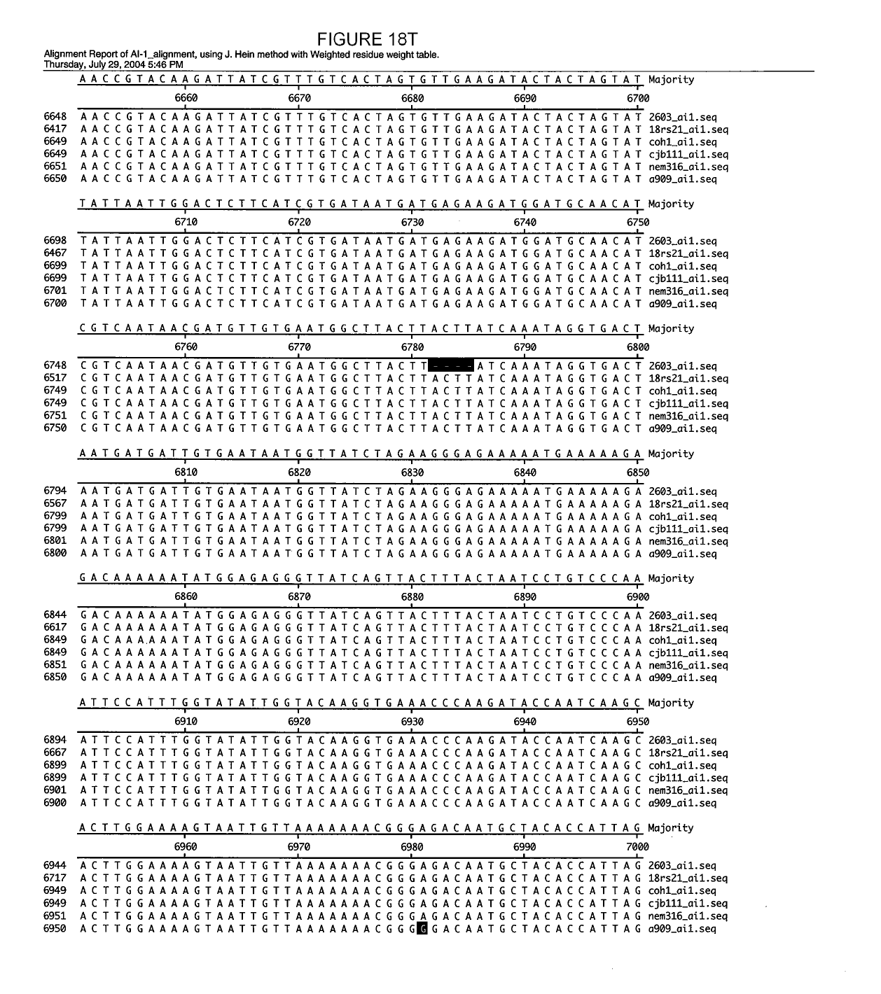

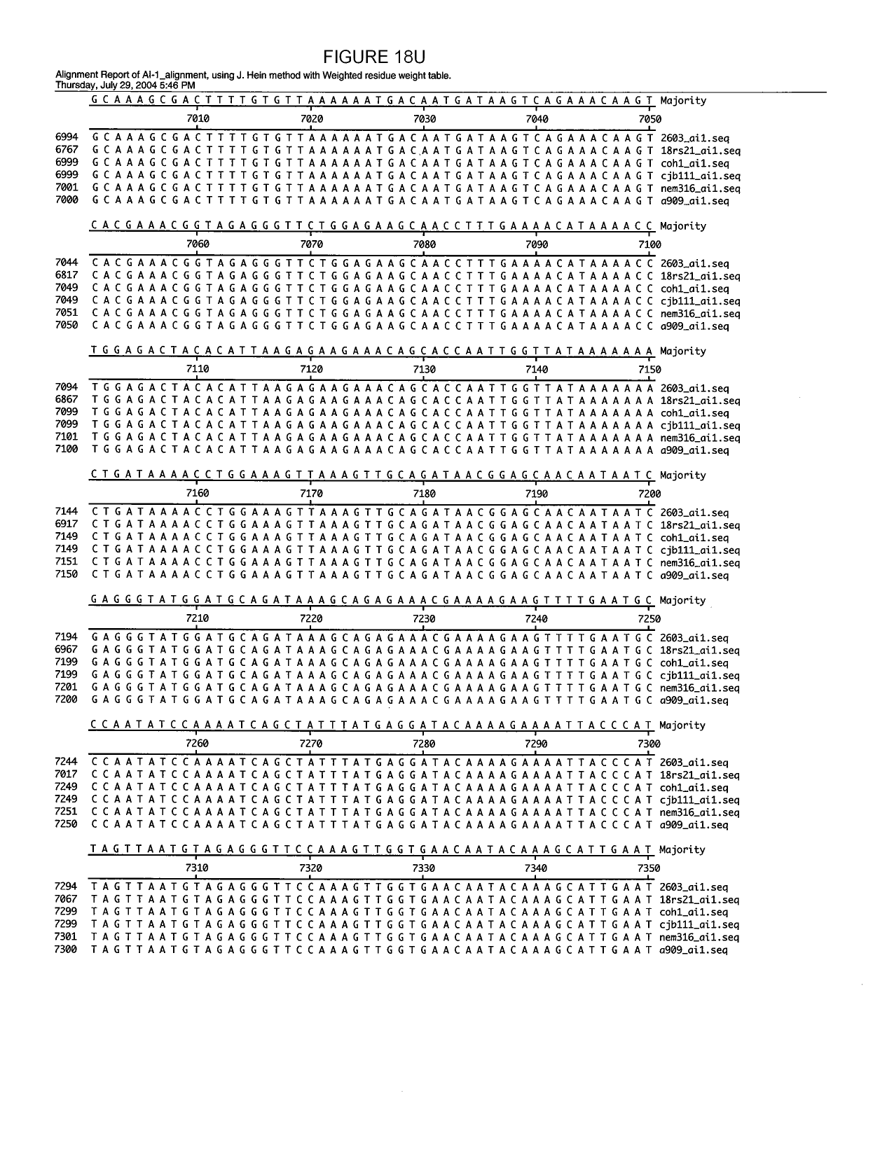

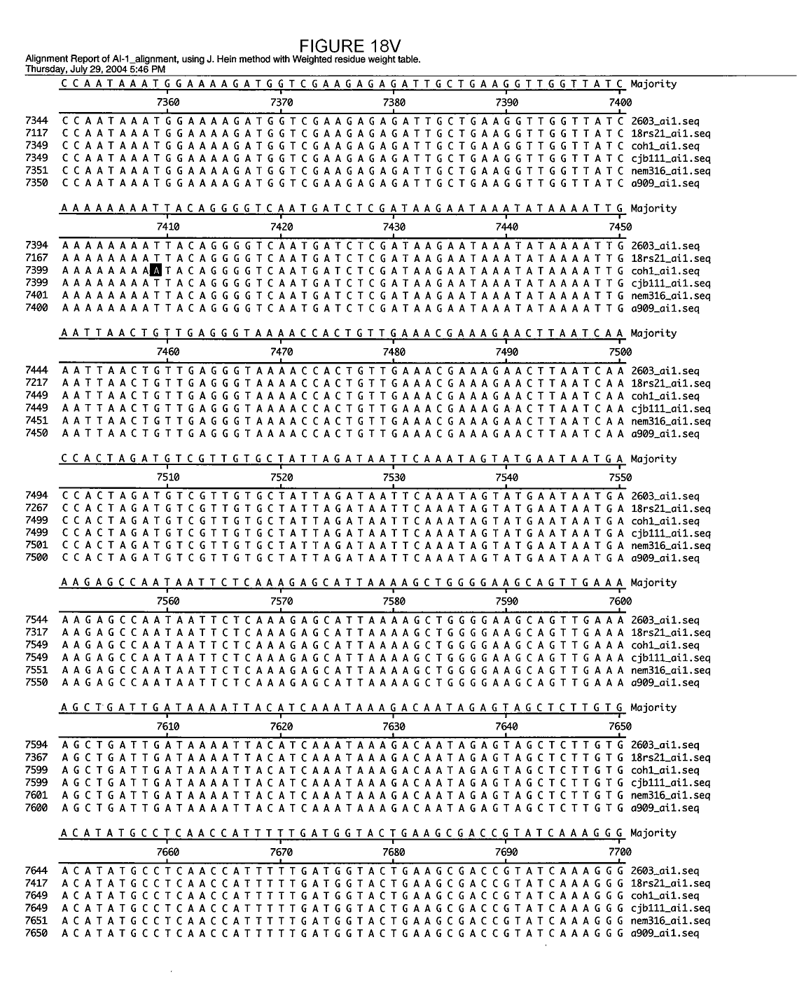

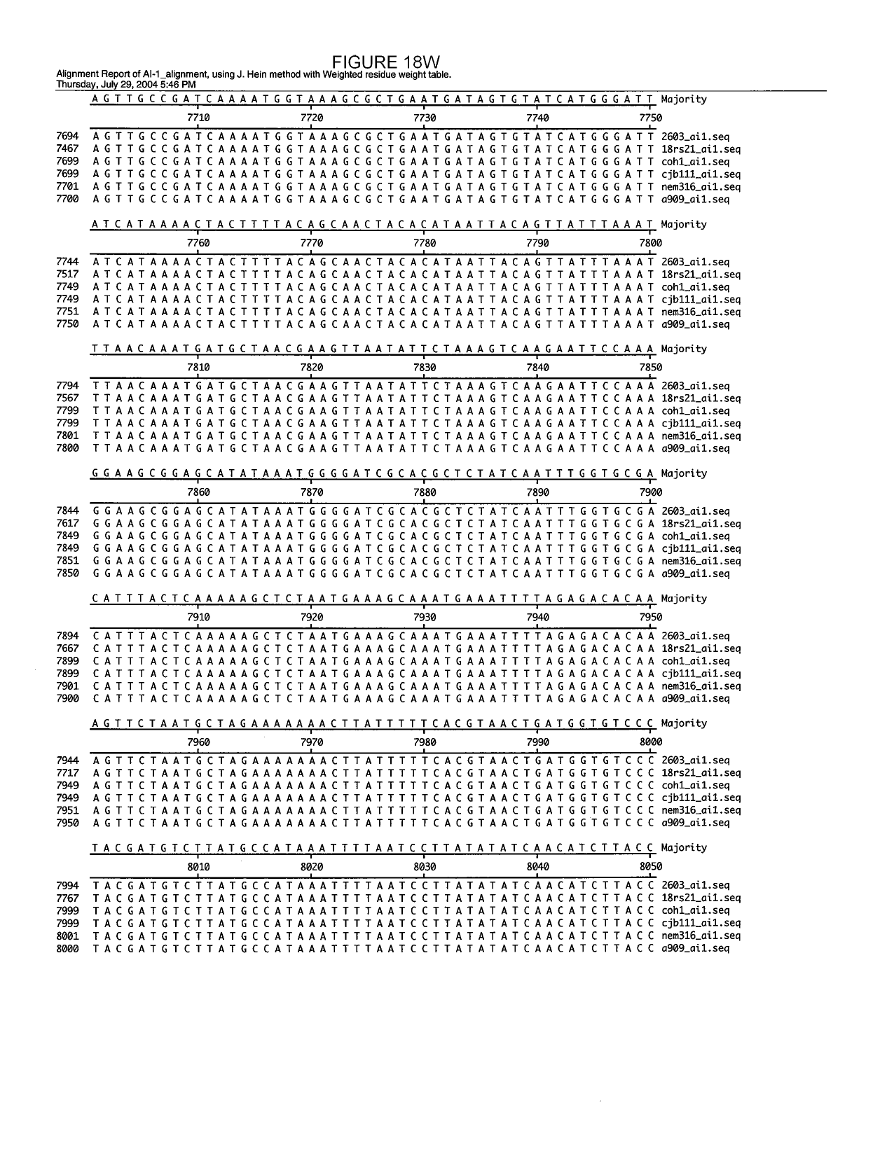

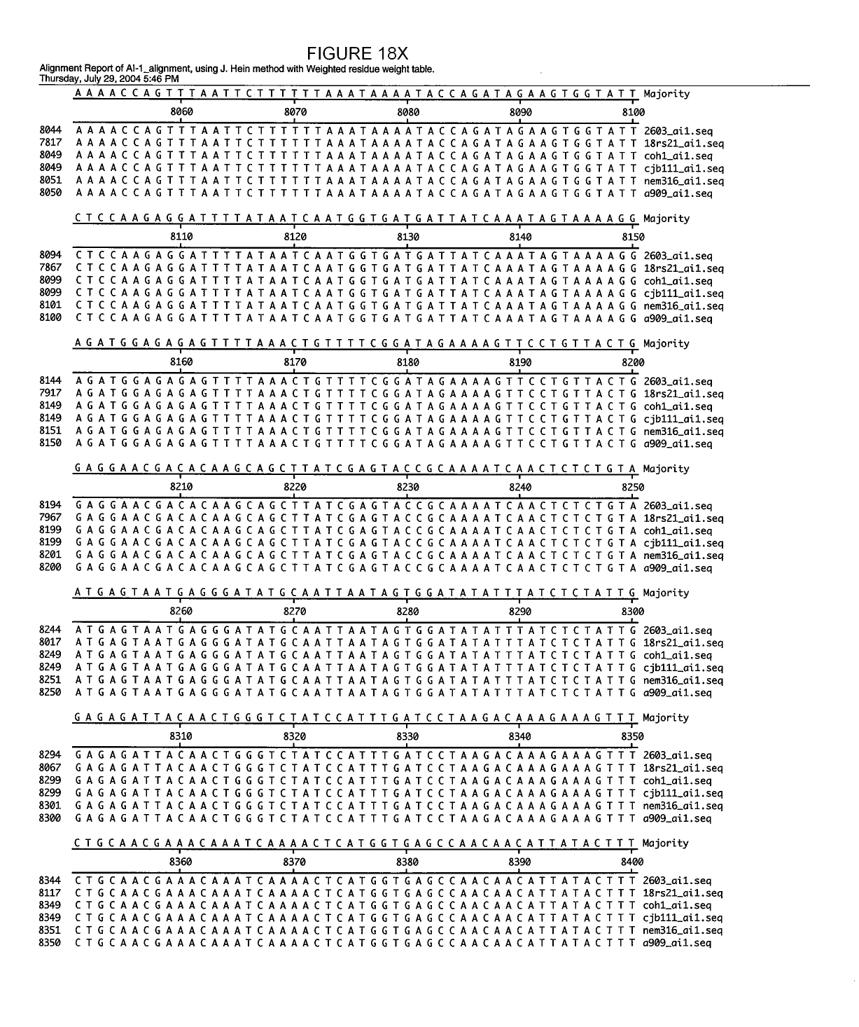

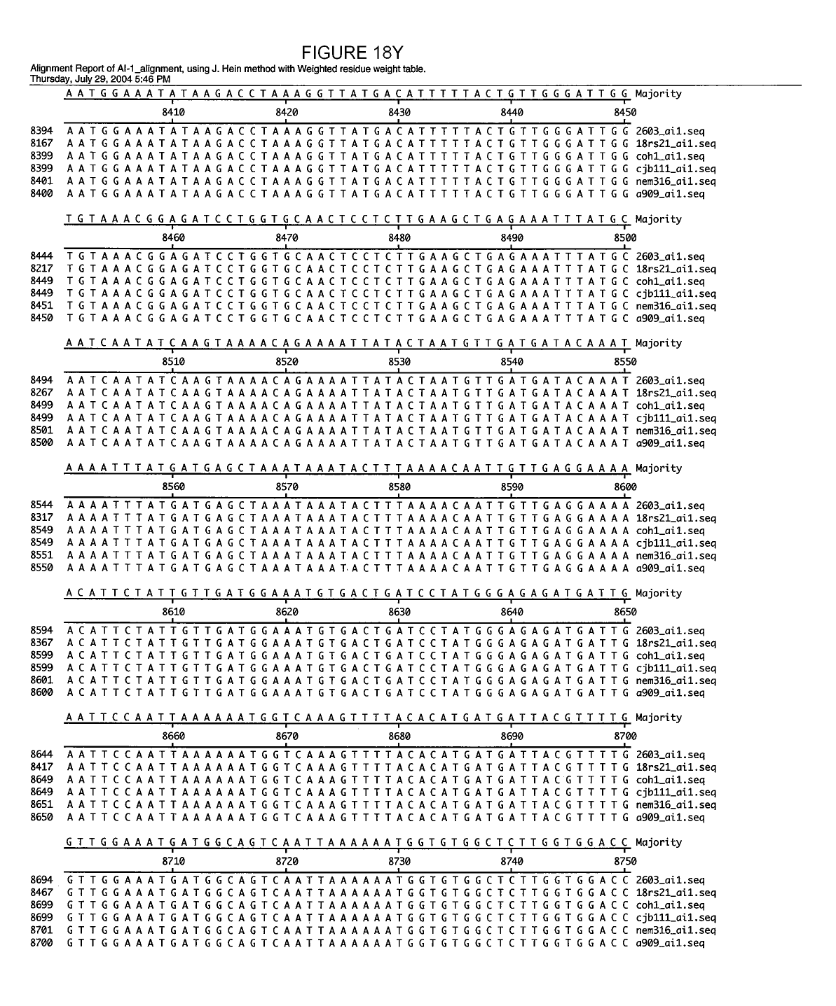

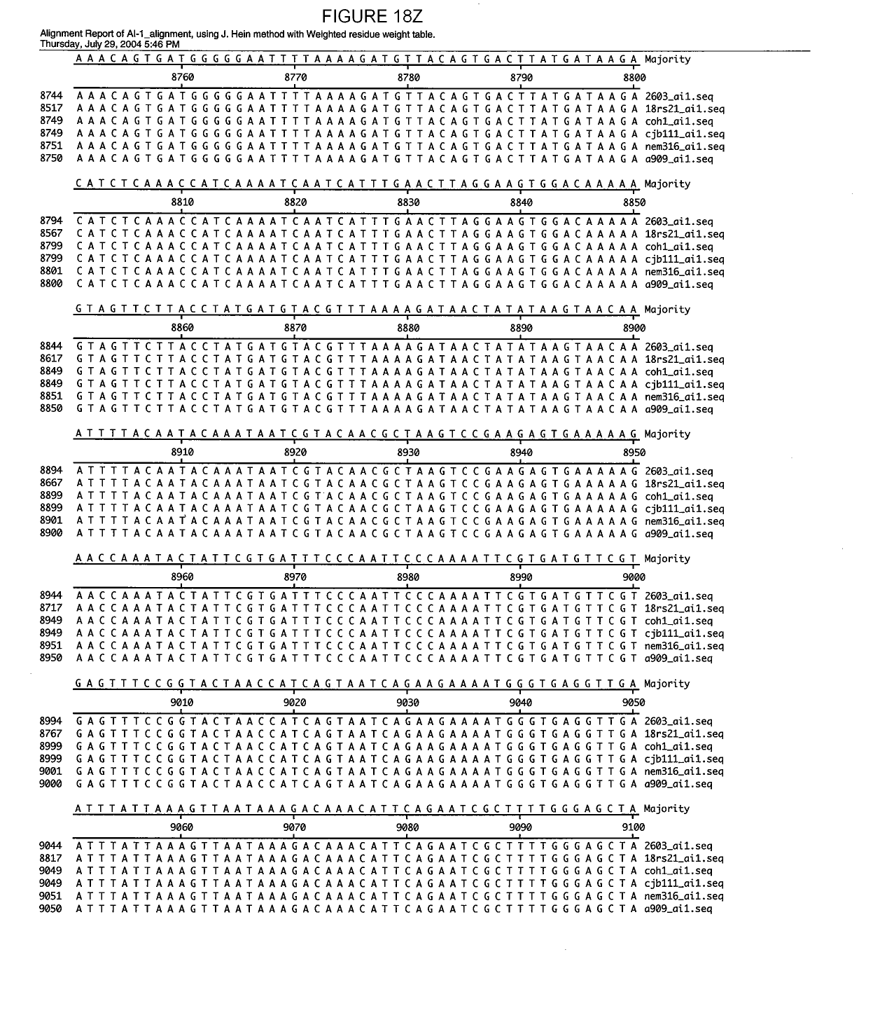

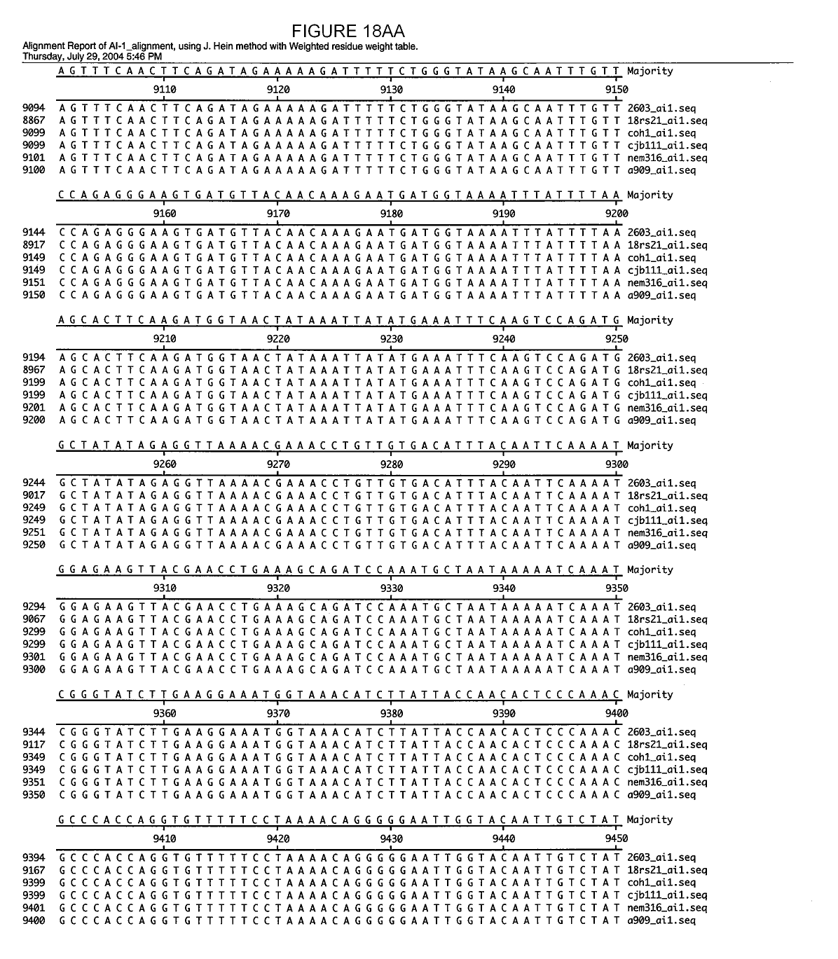

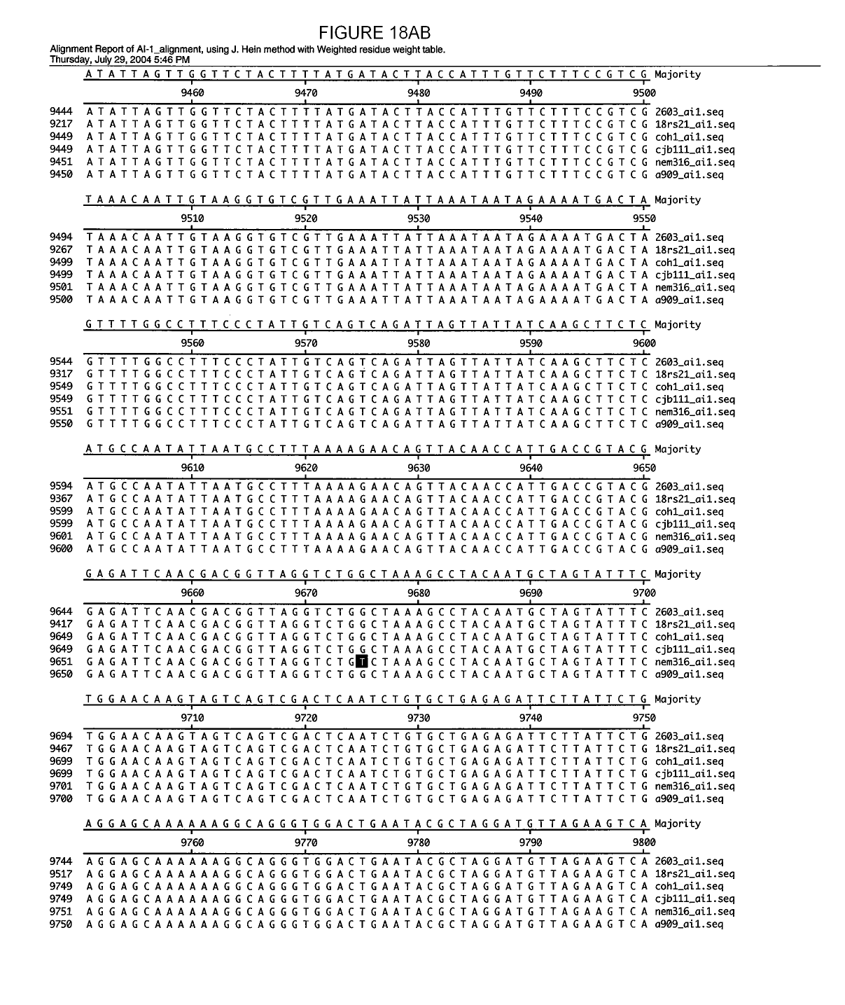

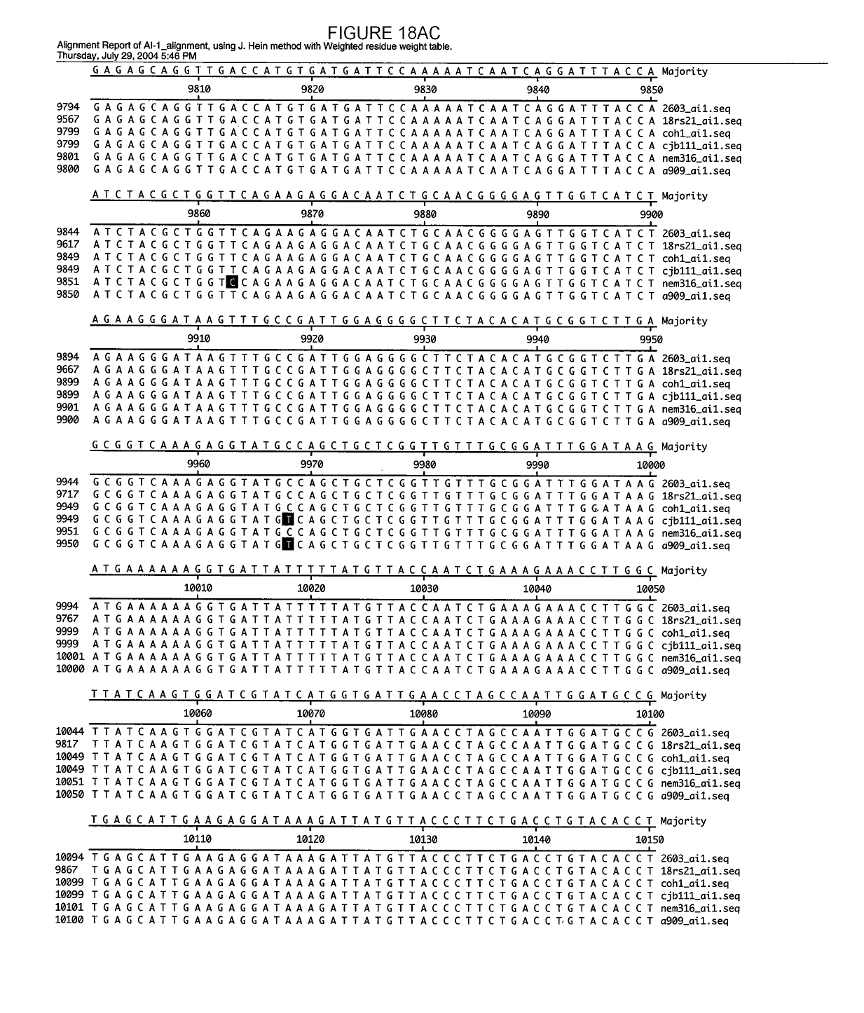

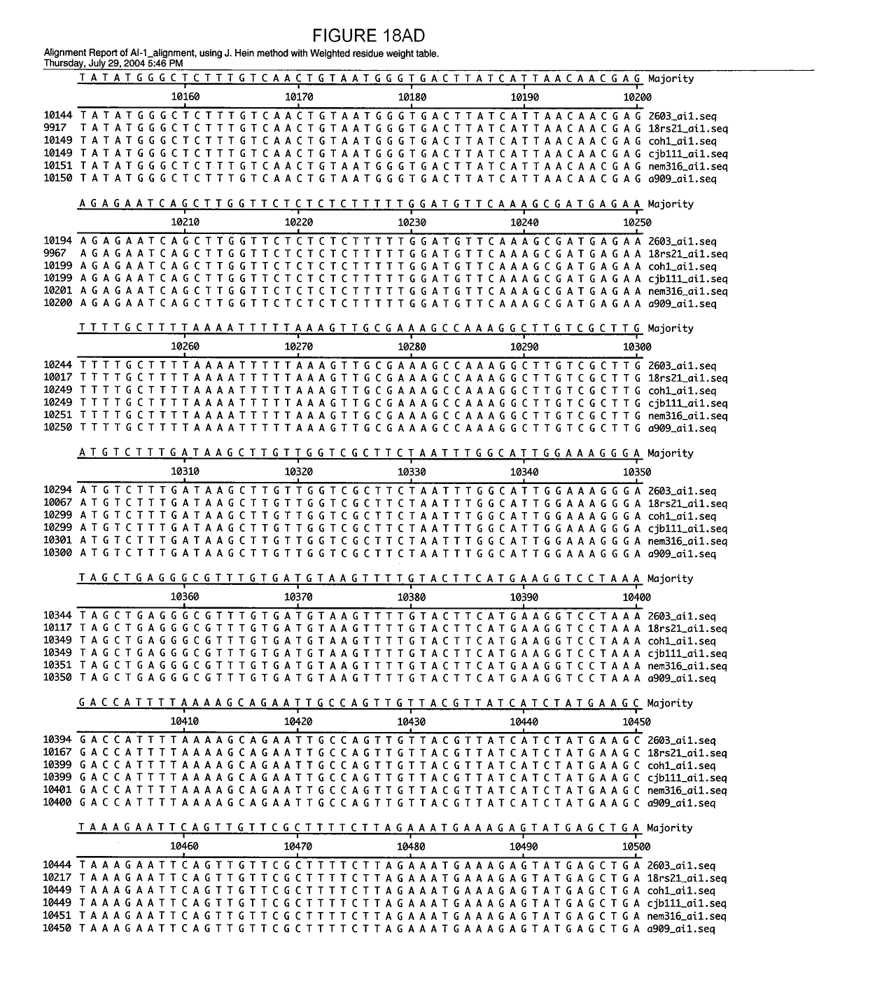

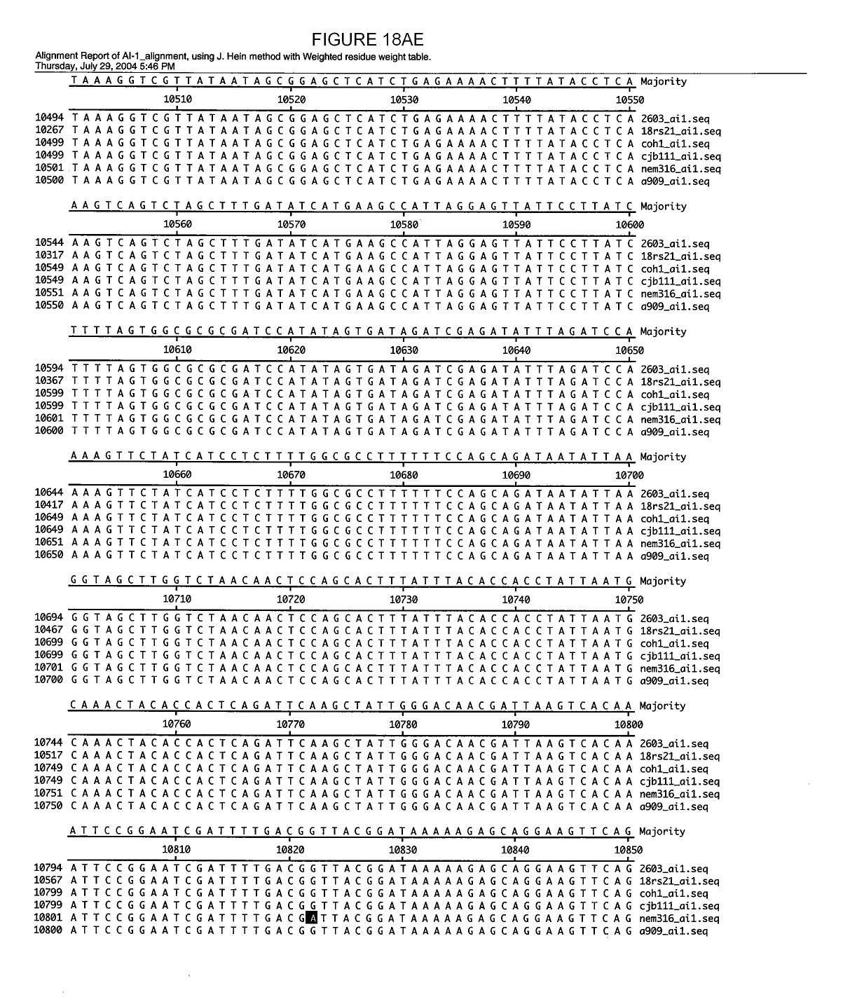

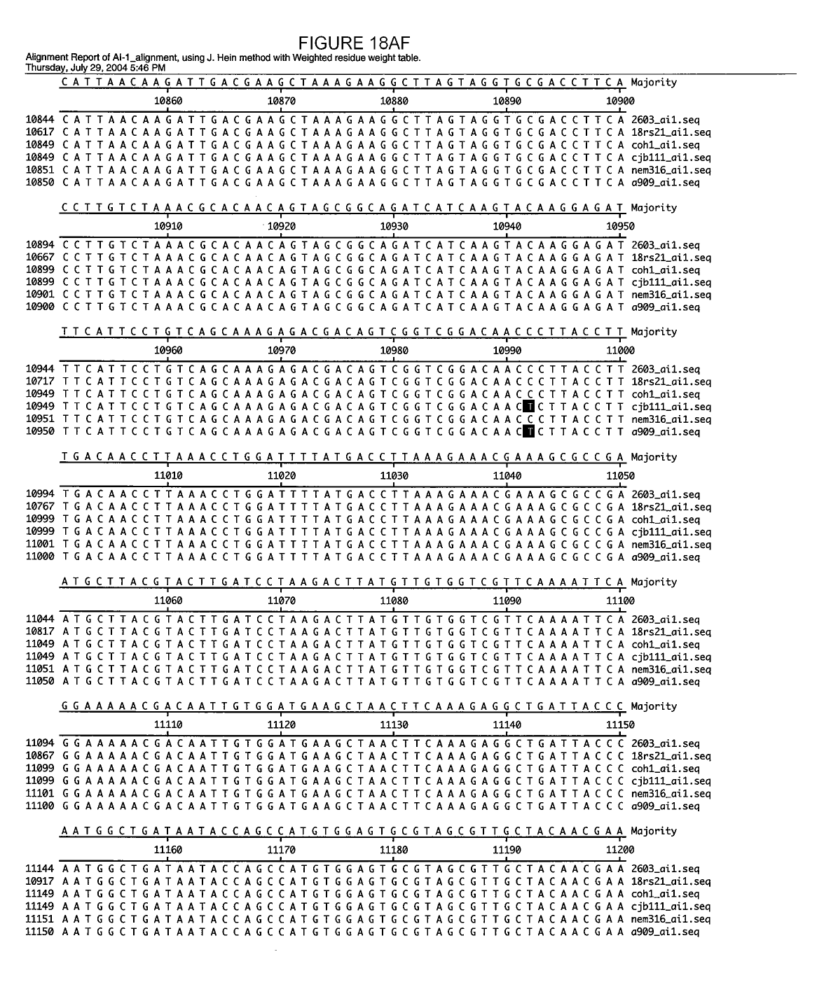

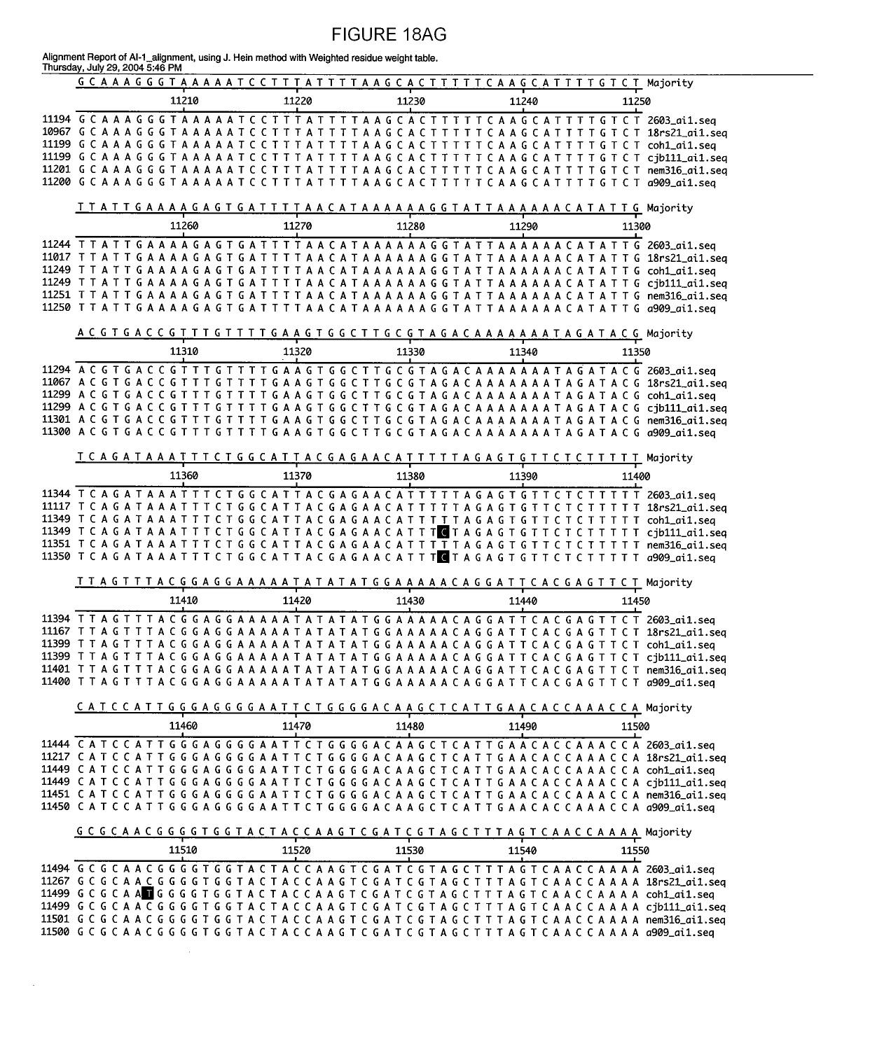

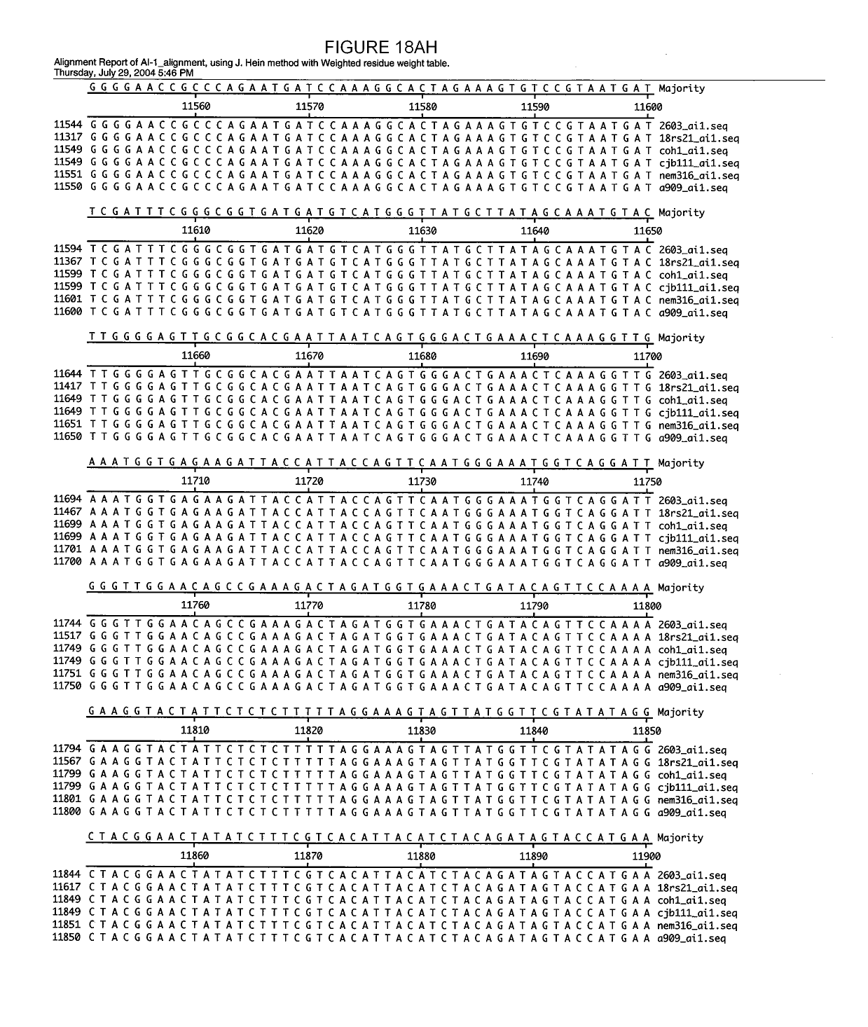

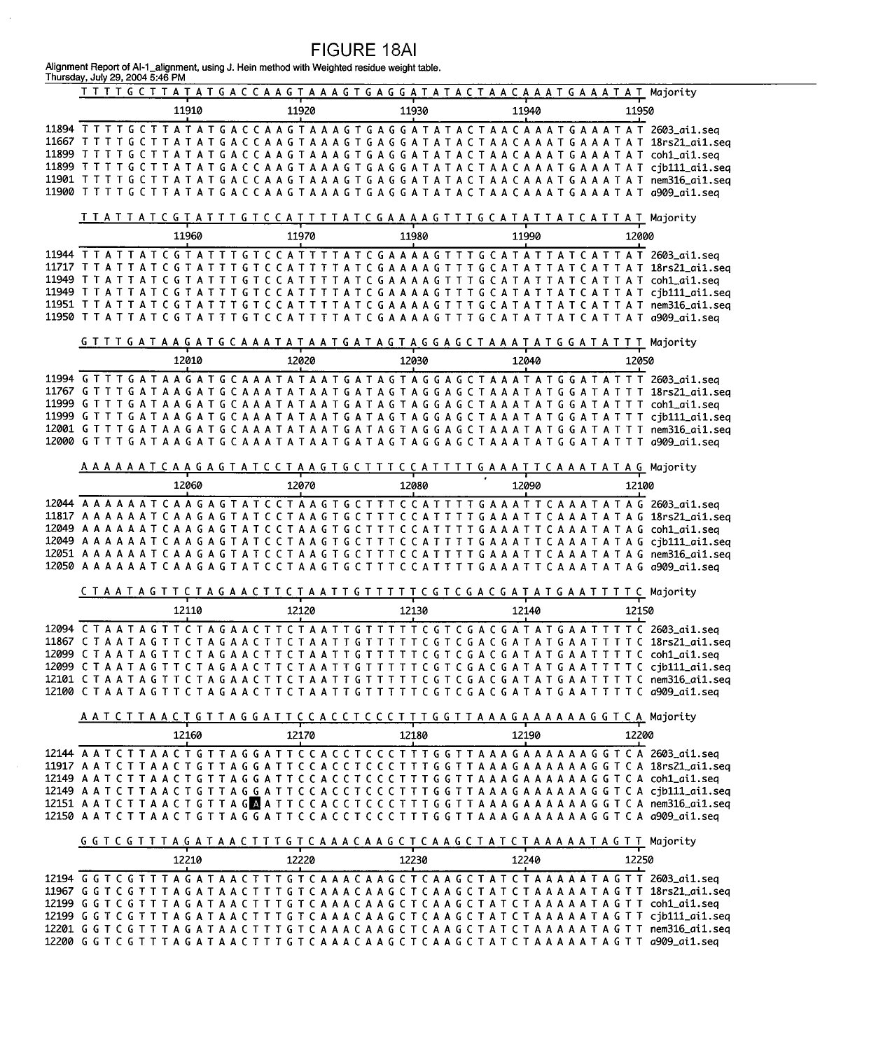

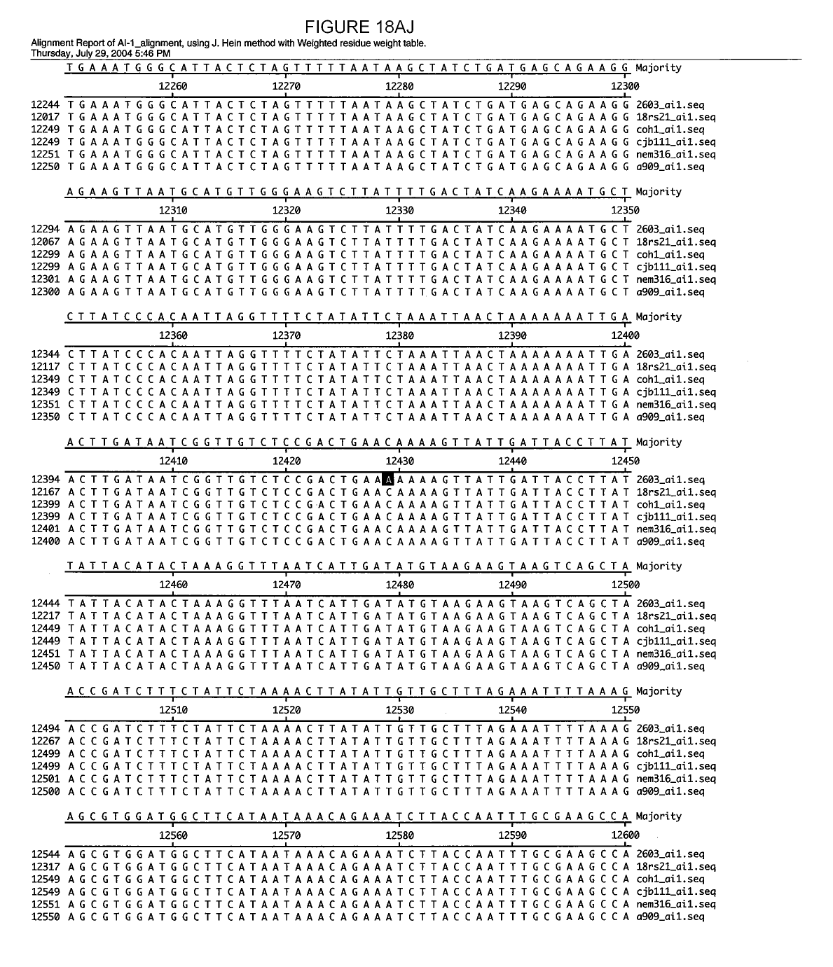

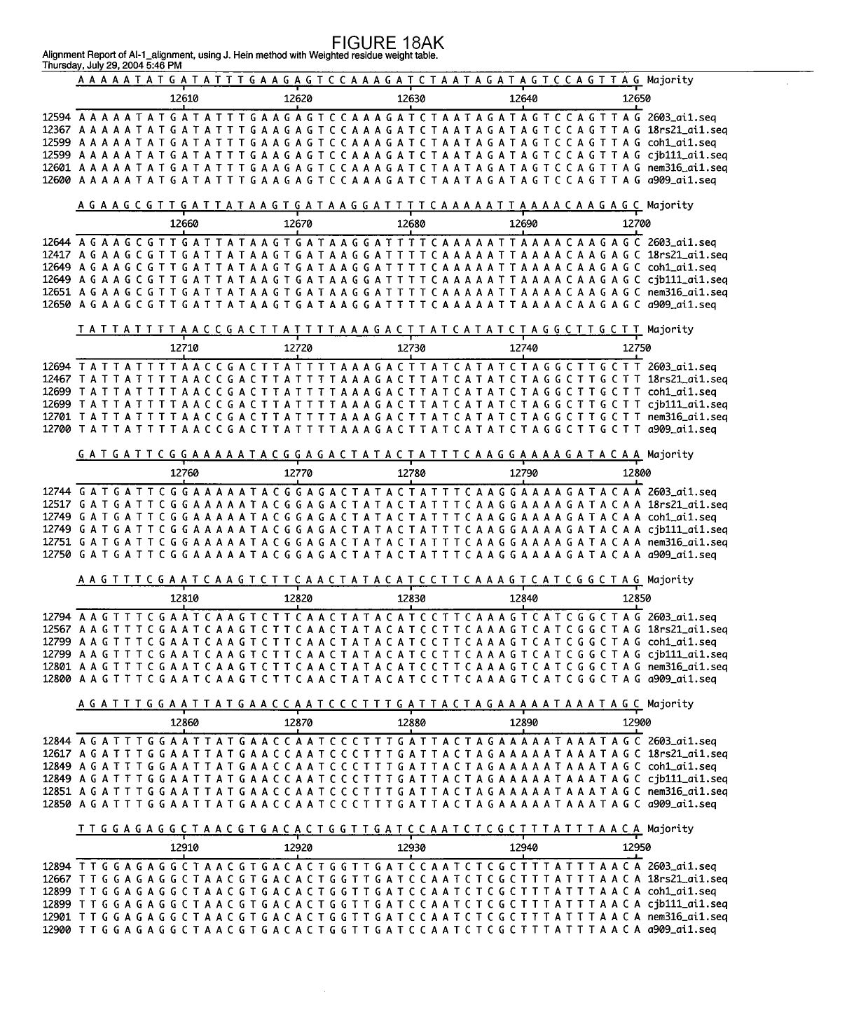

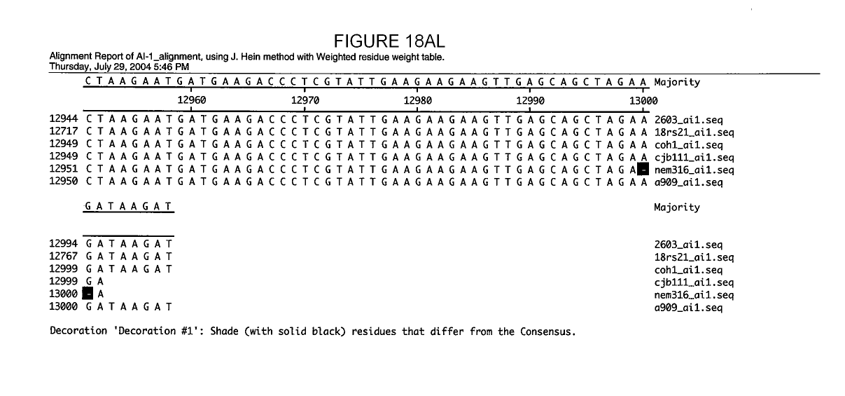

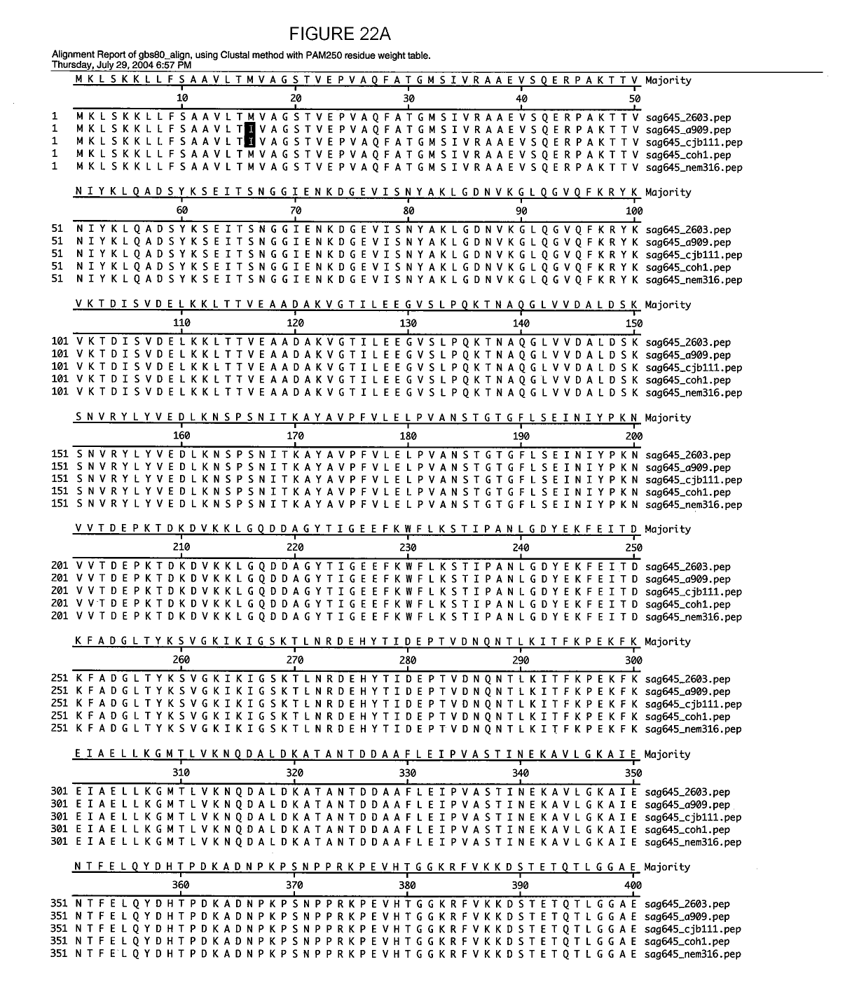

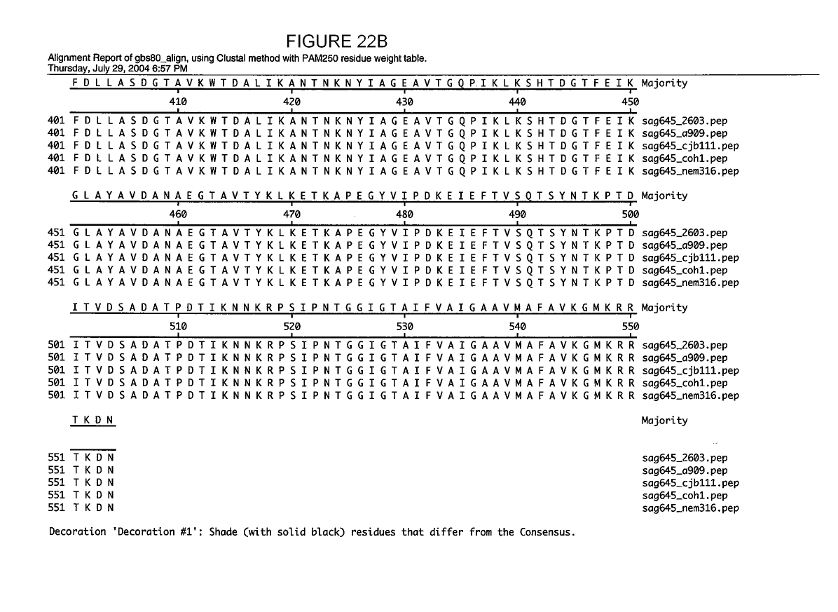

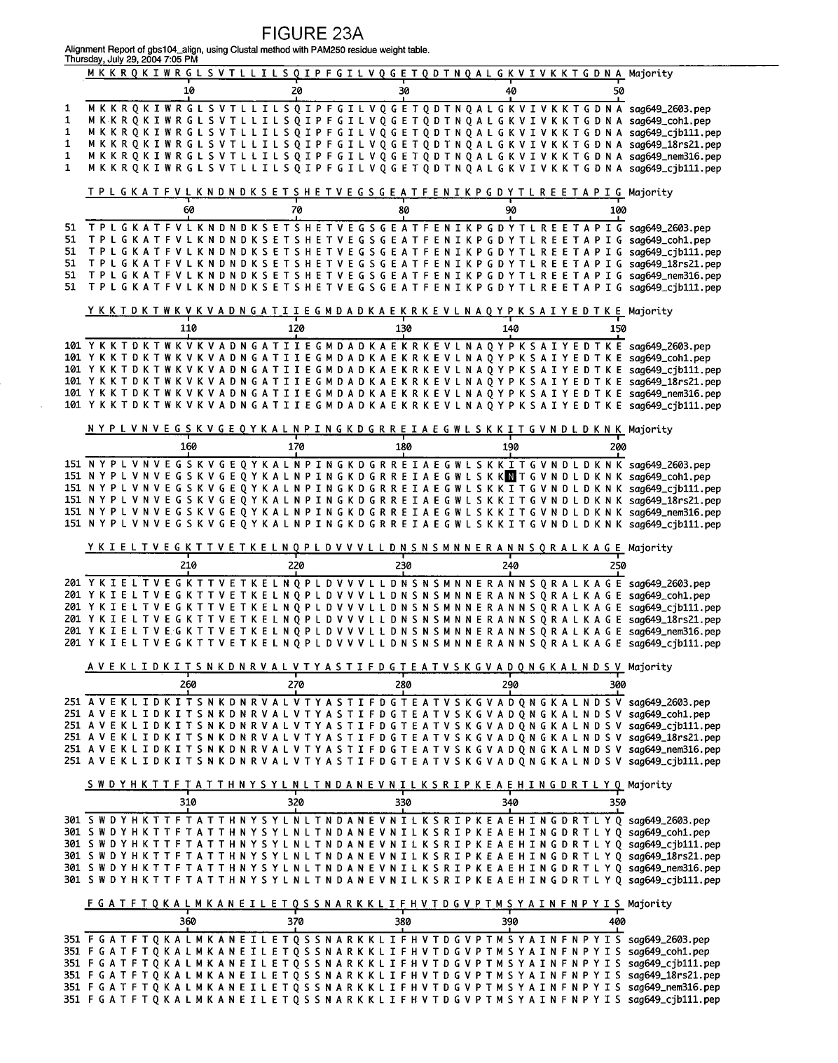

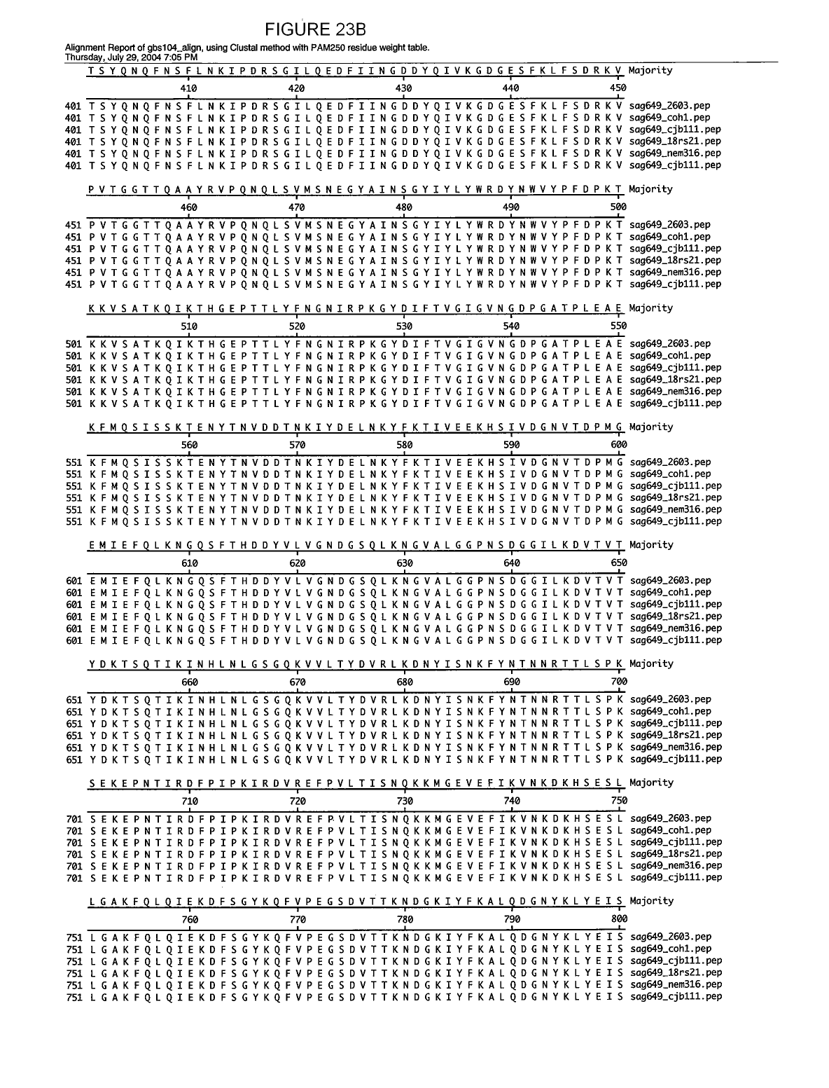

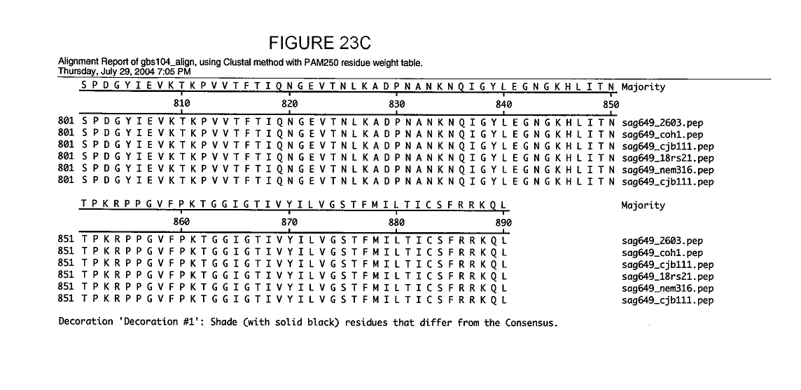

An alignment of AI-1 polynucleotide sequences from serotype V, strain isolates 2603 and CJB111; serotype II, strain isolate 18RS21; serotype III, strain isolates COH1 and NEM316; and serotype 1a, strain isolate A909 is presented in FIG. 18. An alignment of amino acid sequences of AI-1 surface protein GBS 80 from serotype V, strain isolates 2603 and CJB111; serotype 1a, strain isolate A909; serotype III, strain isolates COH1 and NEM316 is presented in FIG. 22. An alignment of amino acid sequences of AI-1 surface protein GBS 104 from serotype V, strain isolates 2603 and CJB111; serotype III, strain isolates COH1 and NEM316; and serotype II, strain isolate 18RS21 is presented in FIG. 23. Preferred AI-1 polynucleotide and amino acid sequences are conserved among two or more GBS serotypes or strain isolates.

The full length of surface protein GBS 80 is particularly conserved among GBS serotypes V (strain isolates 2603 and CJBIII), III (strain isolates NEM316 and COH1), and Ia (strain isolate A909). The GBS 80 surface protein is missing or fragmented in serotypes II (strain isolate 18RS21), Ib (strain isolate H36B) and Ia (strain isolate 515).

Polynucleotide and amino acid sequences for AraC are set forth in FIG. 30.

GBS Adhesin Island 2

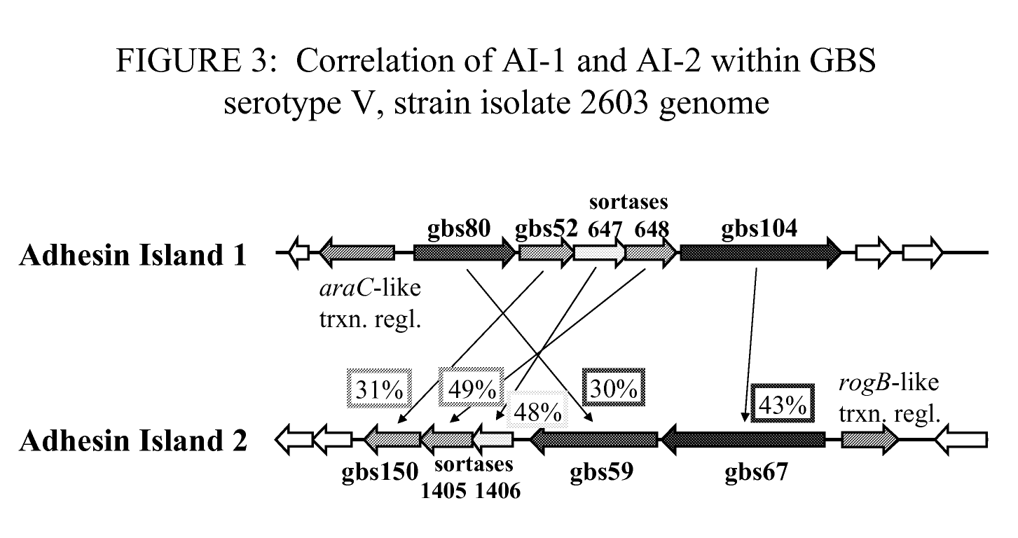

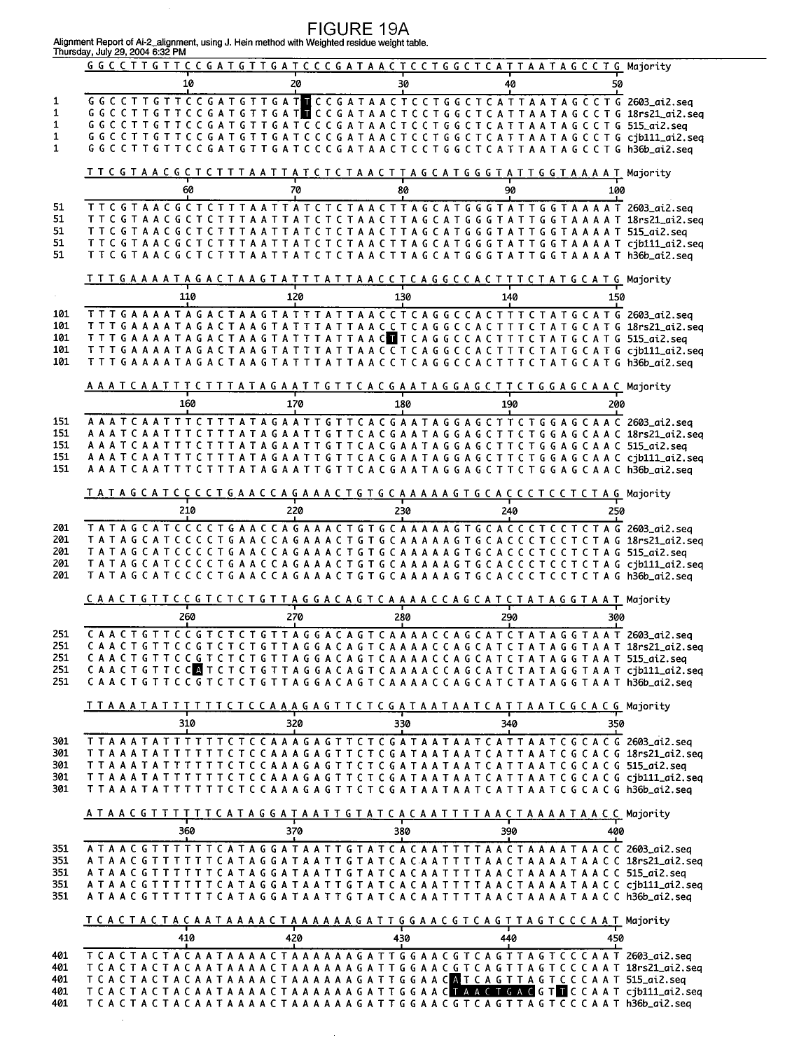

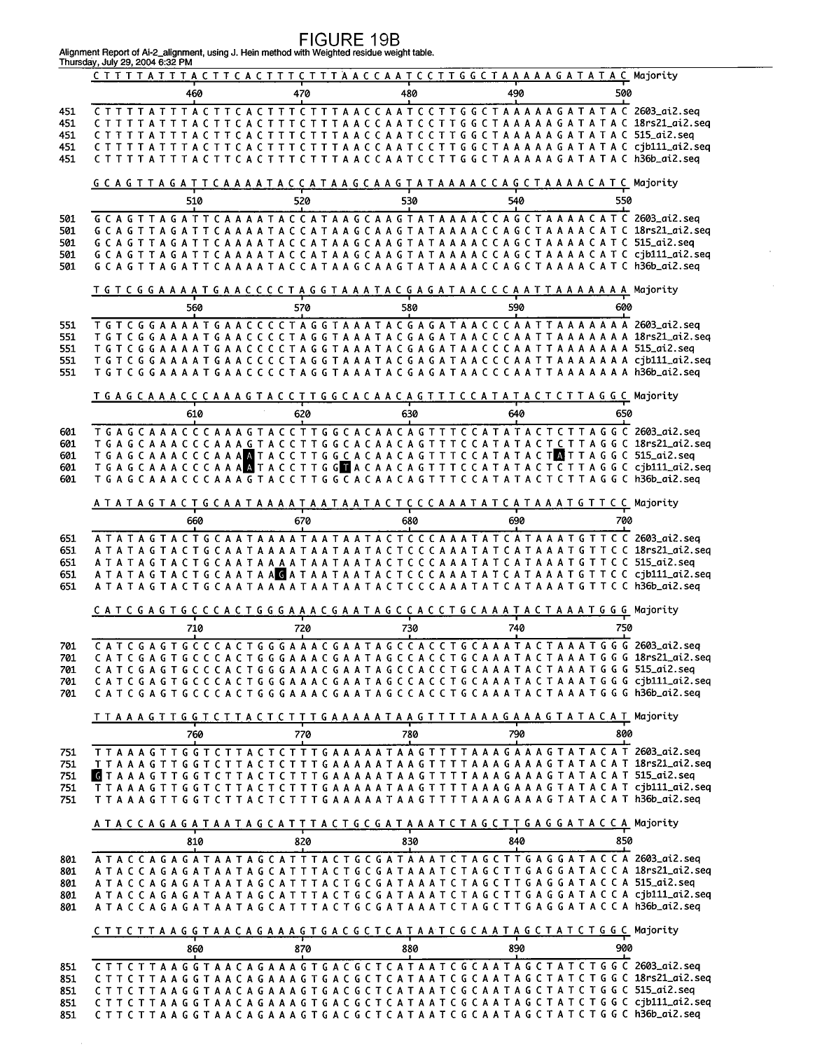

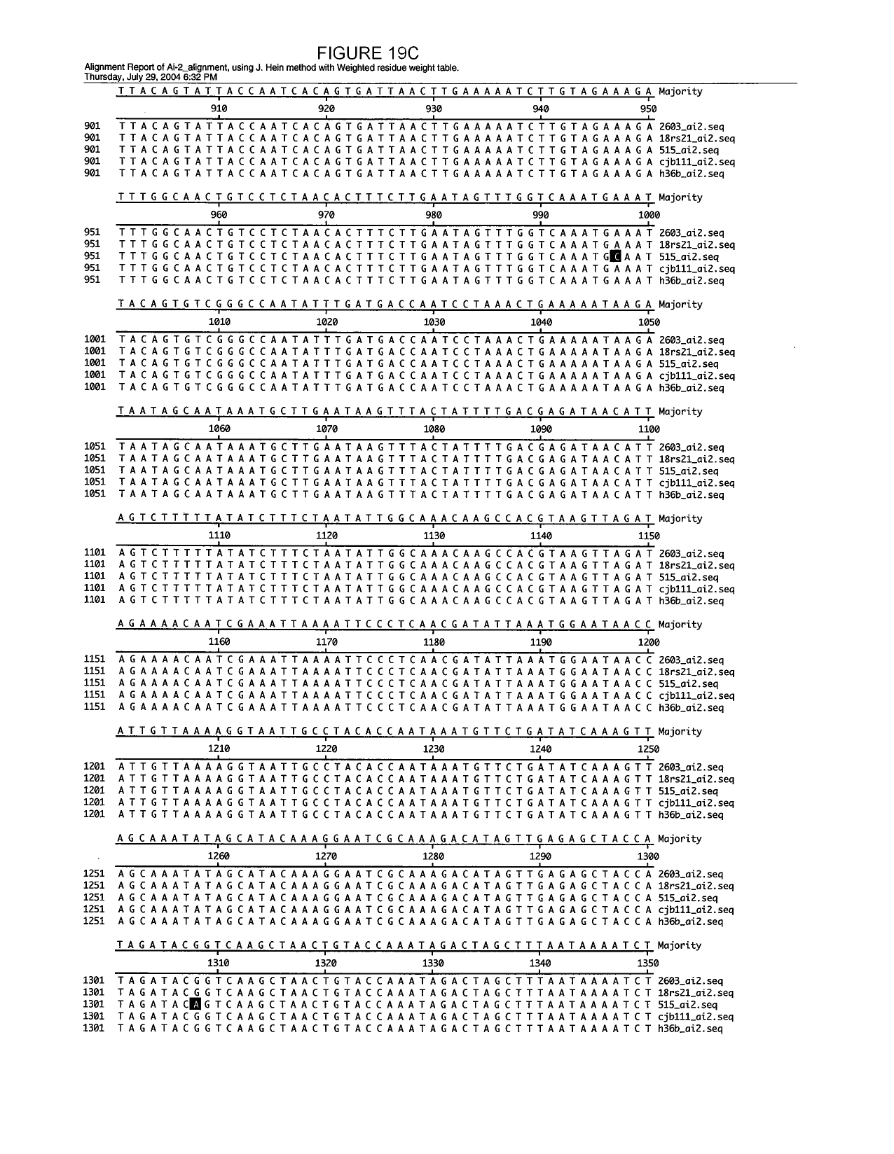

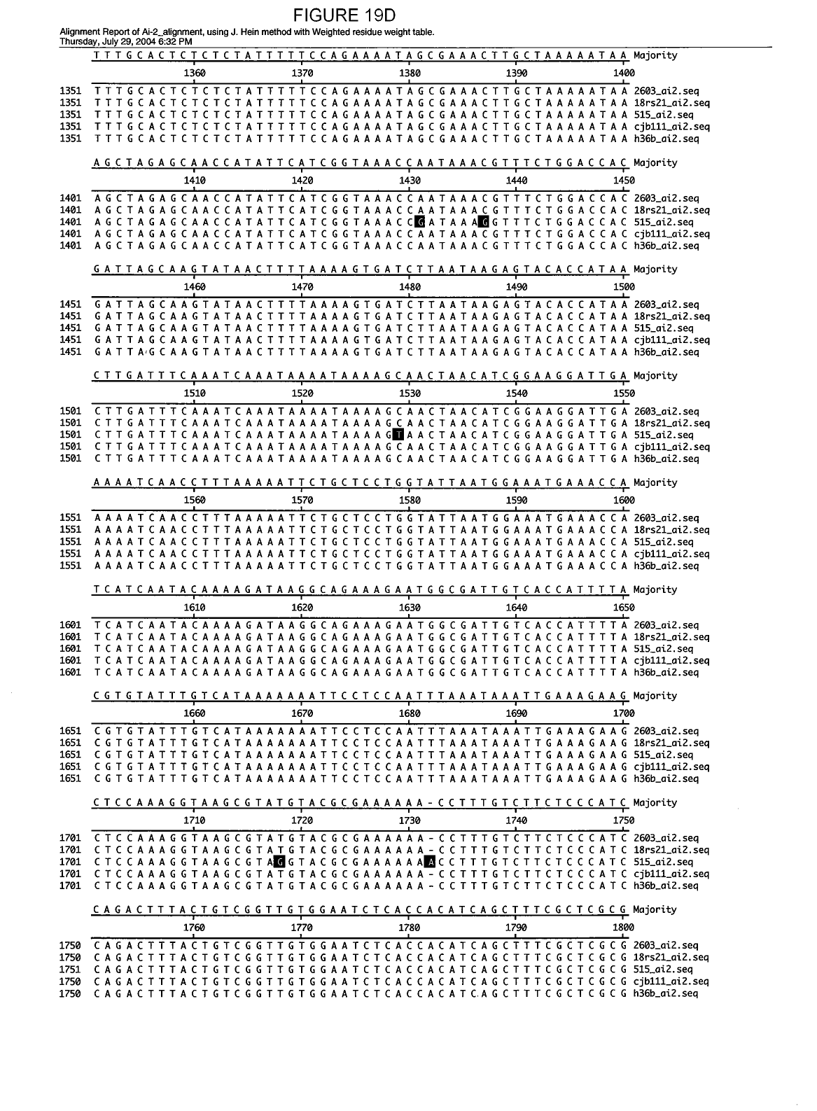

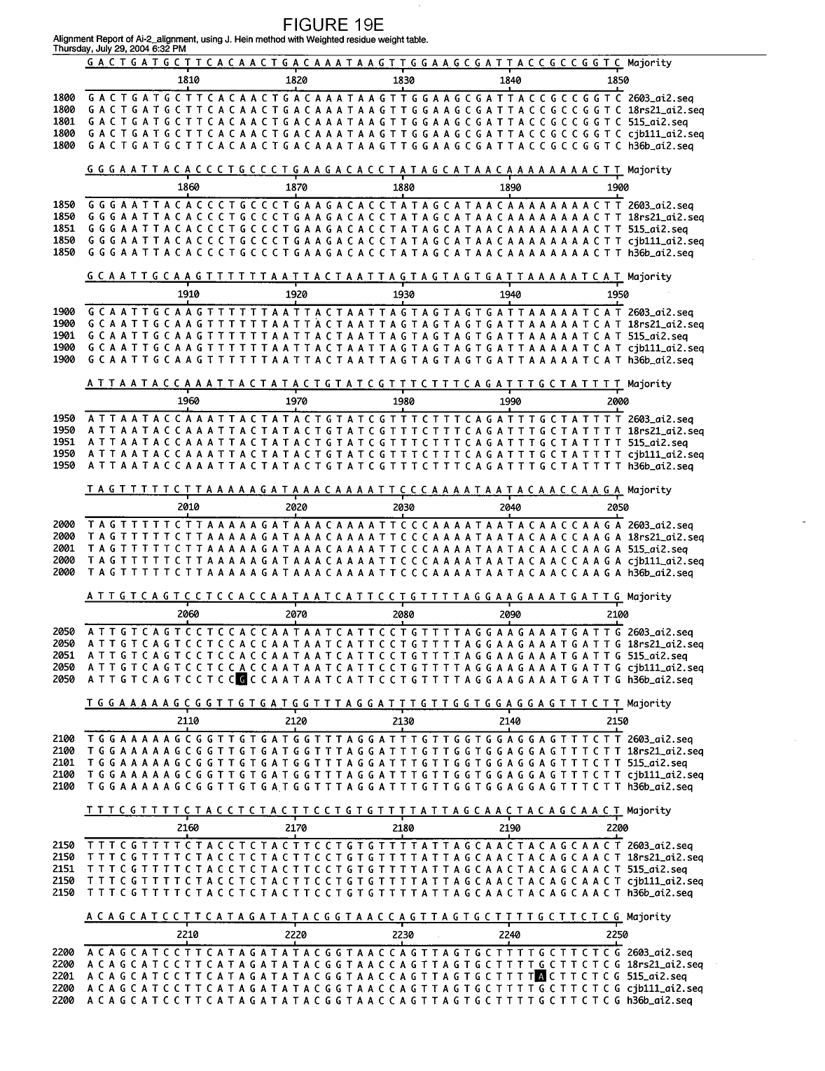

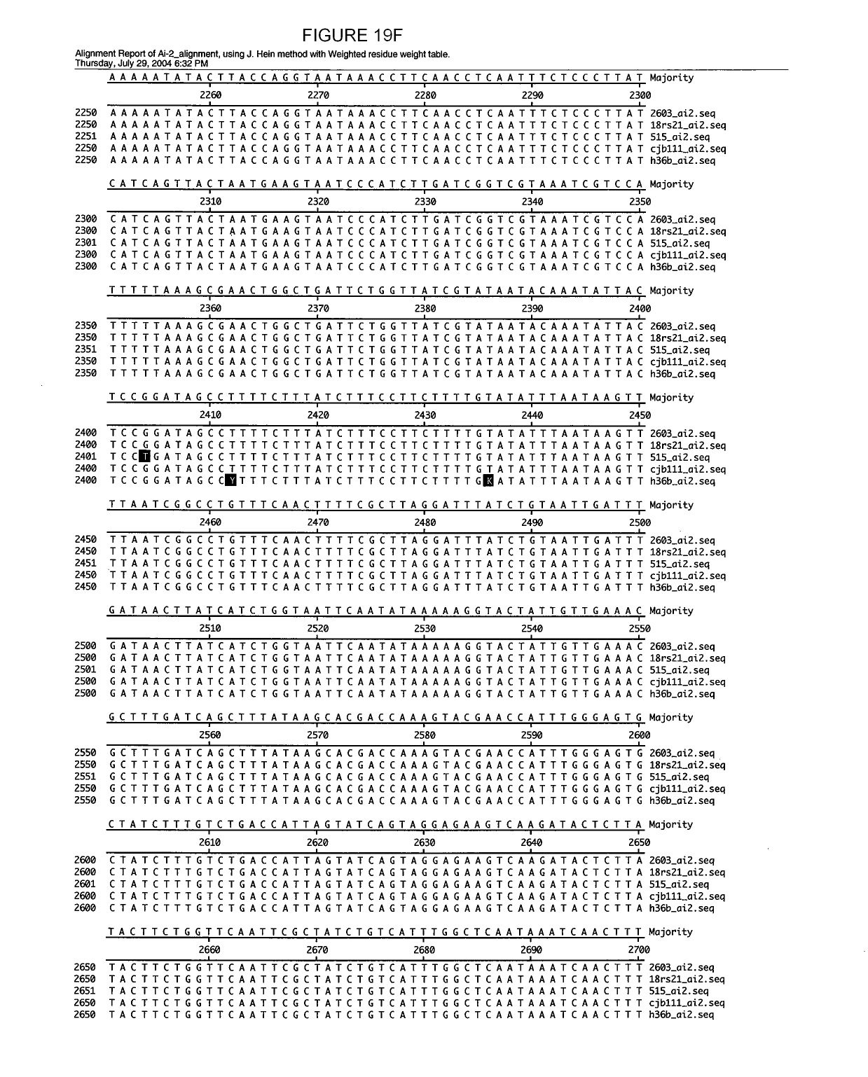

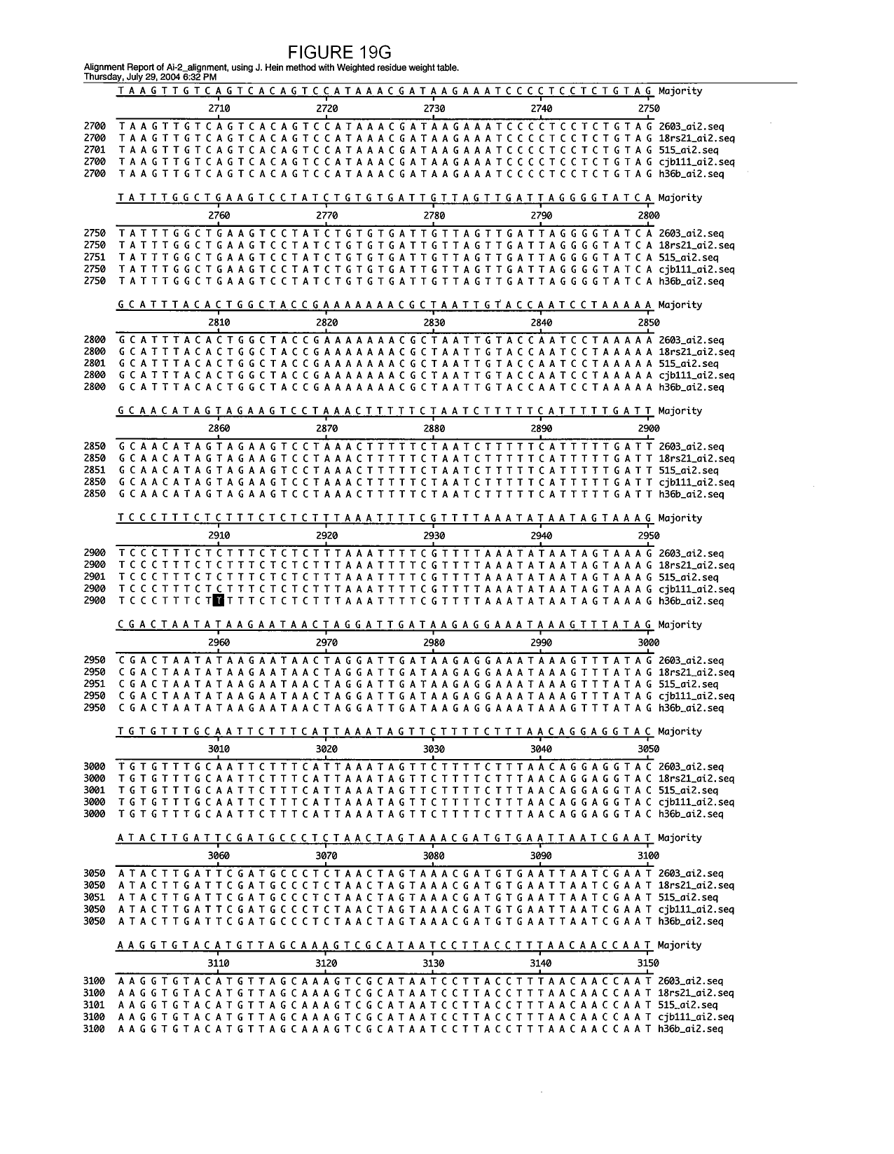

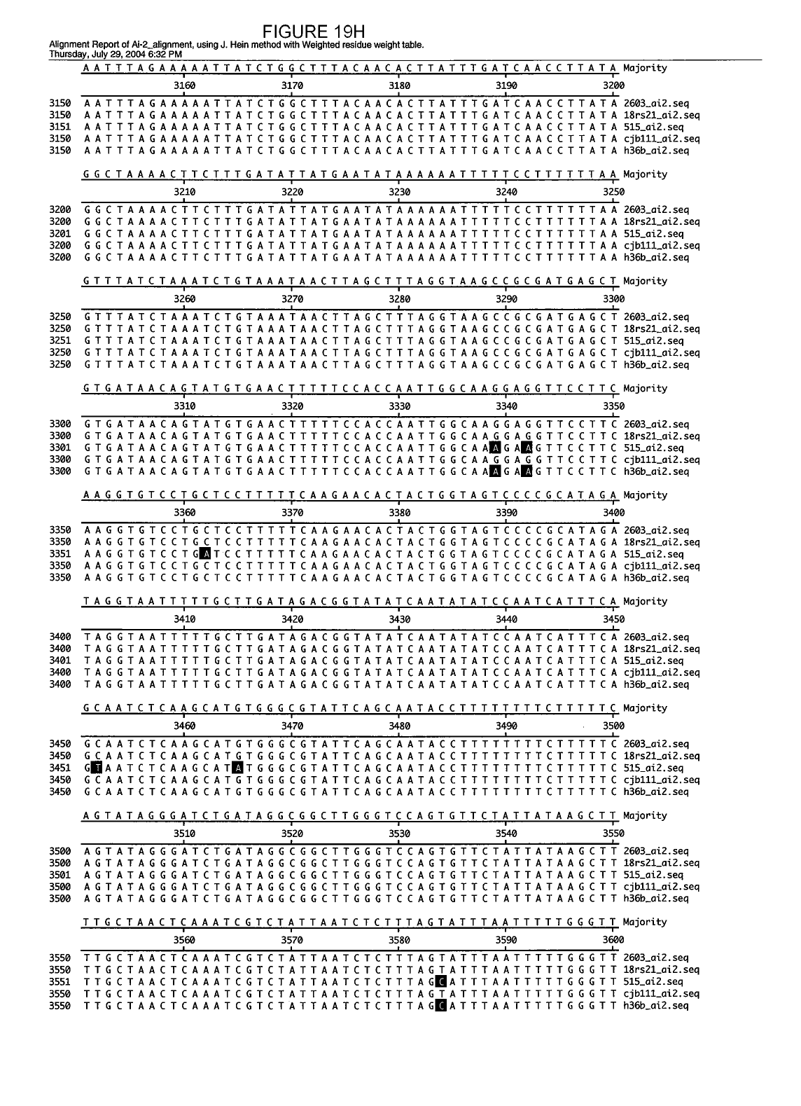

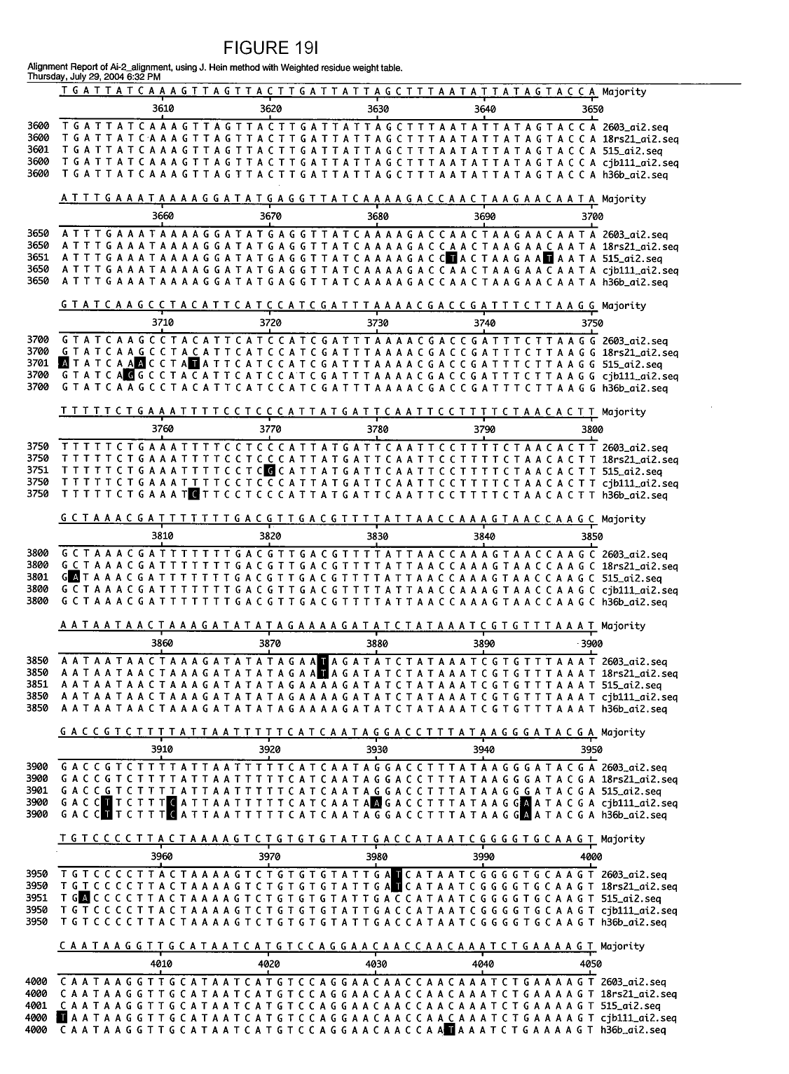

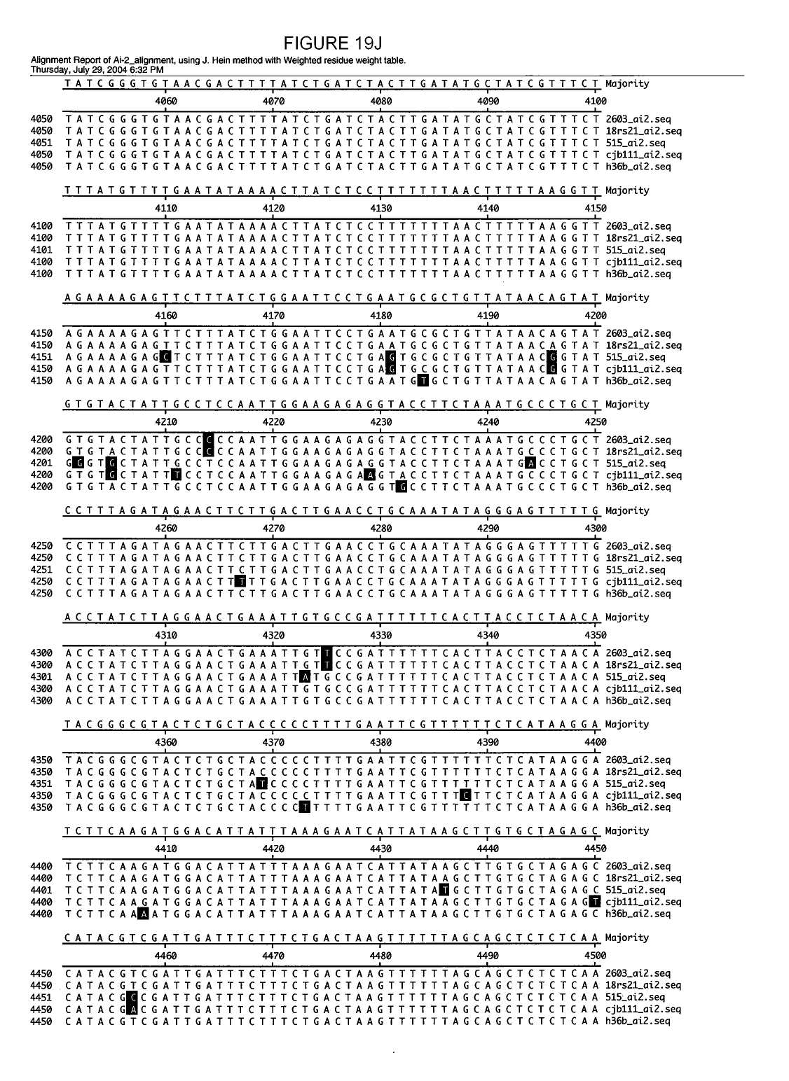

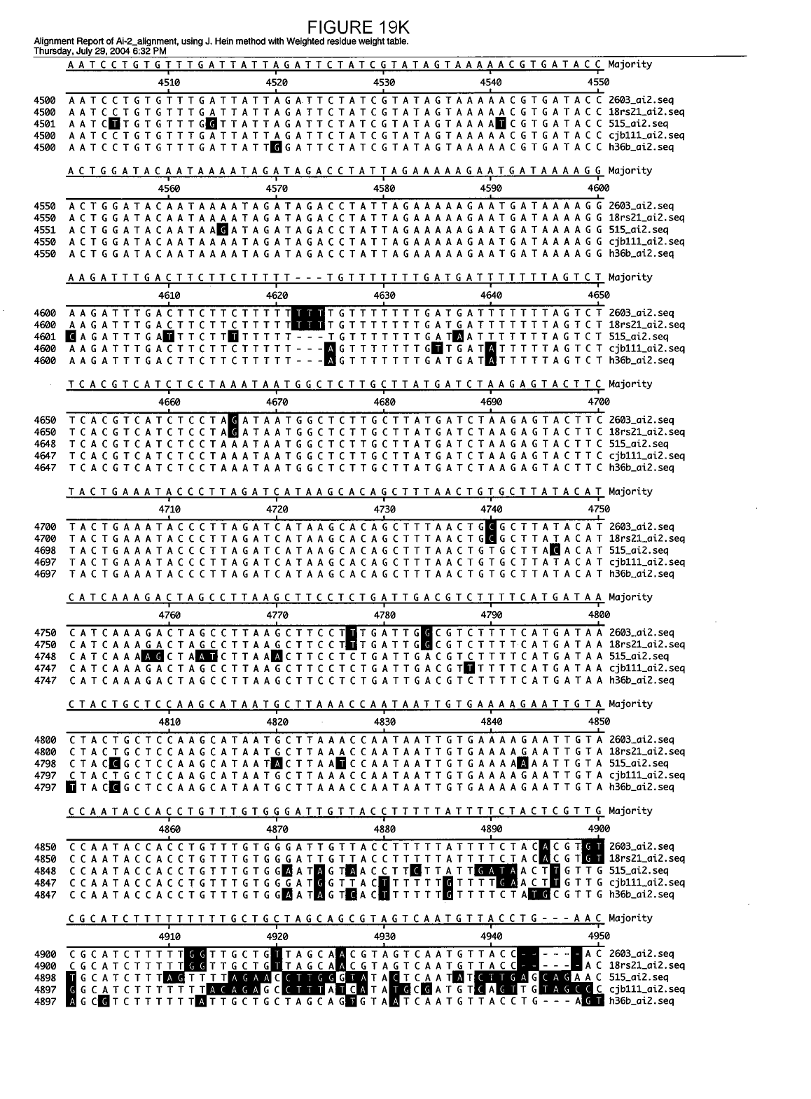

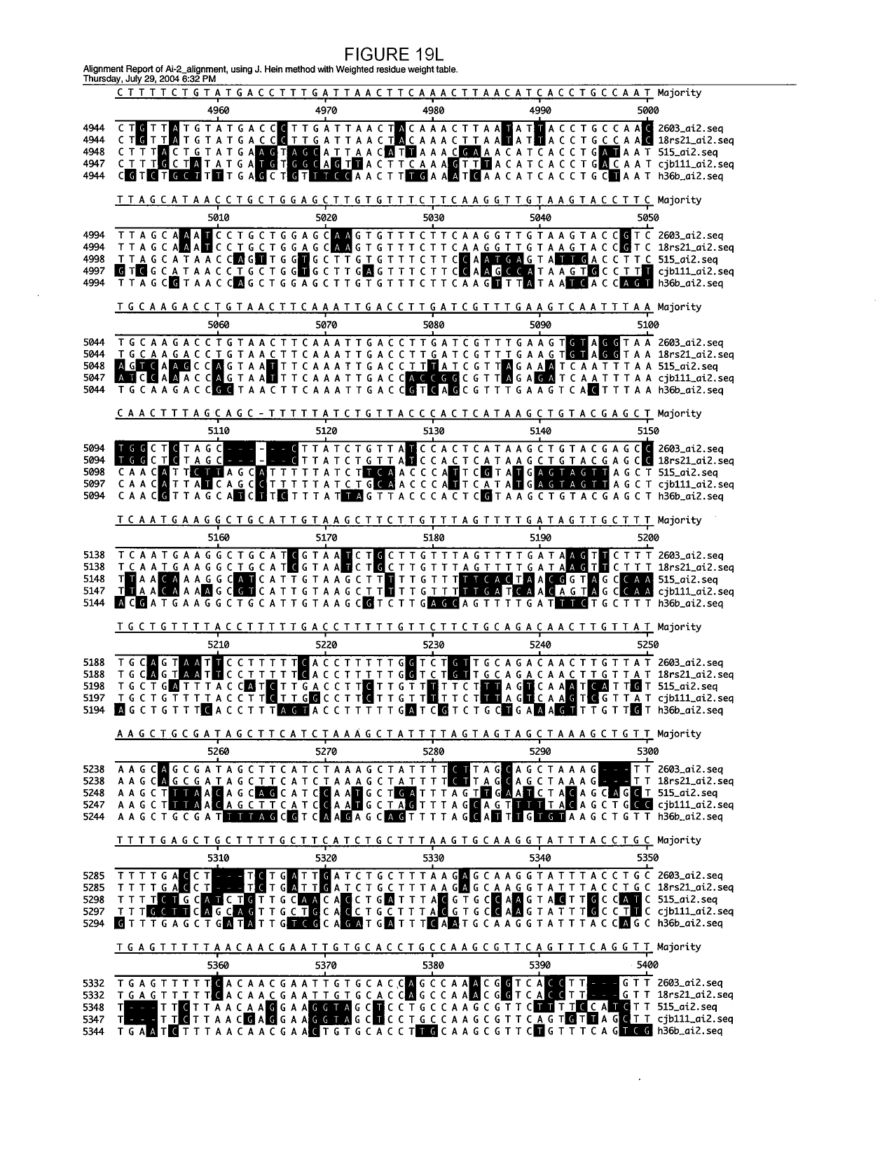

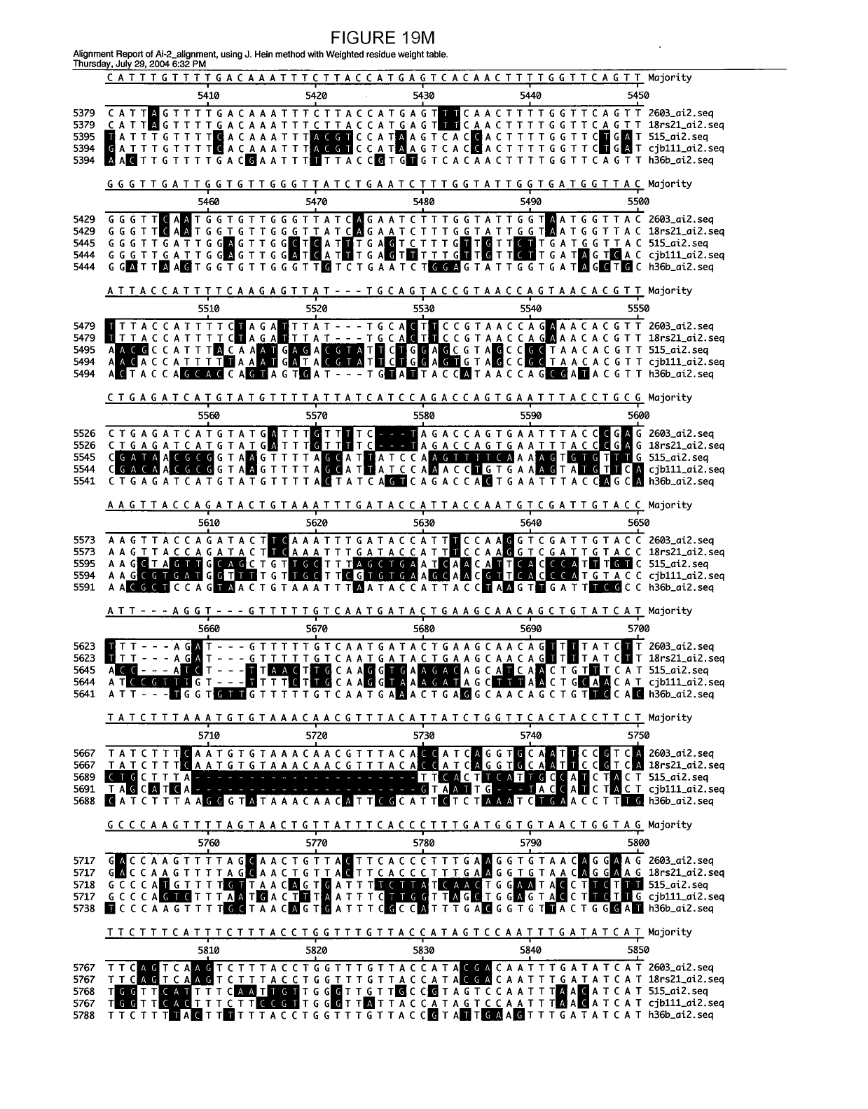

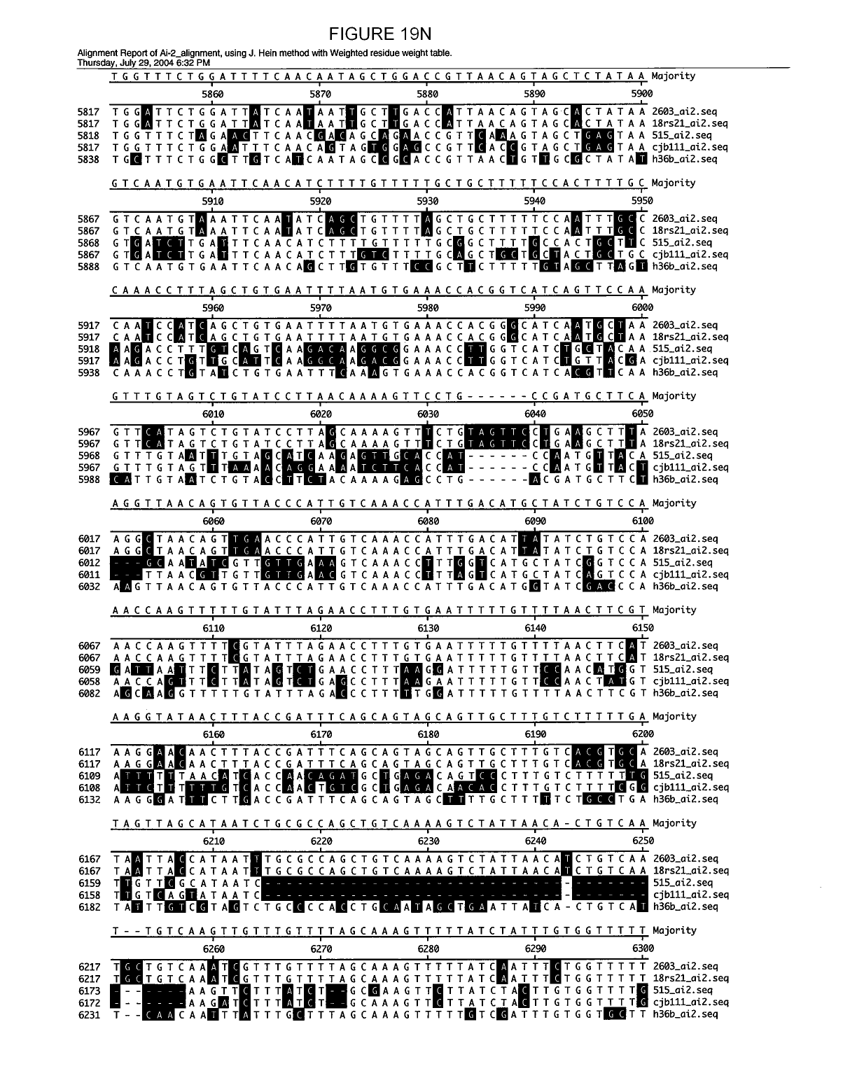

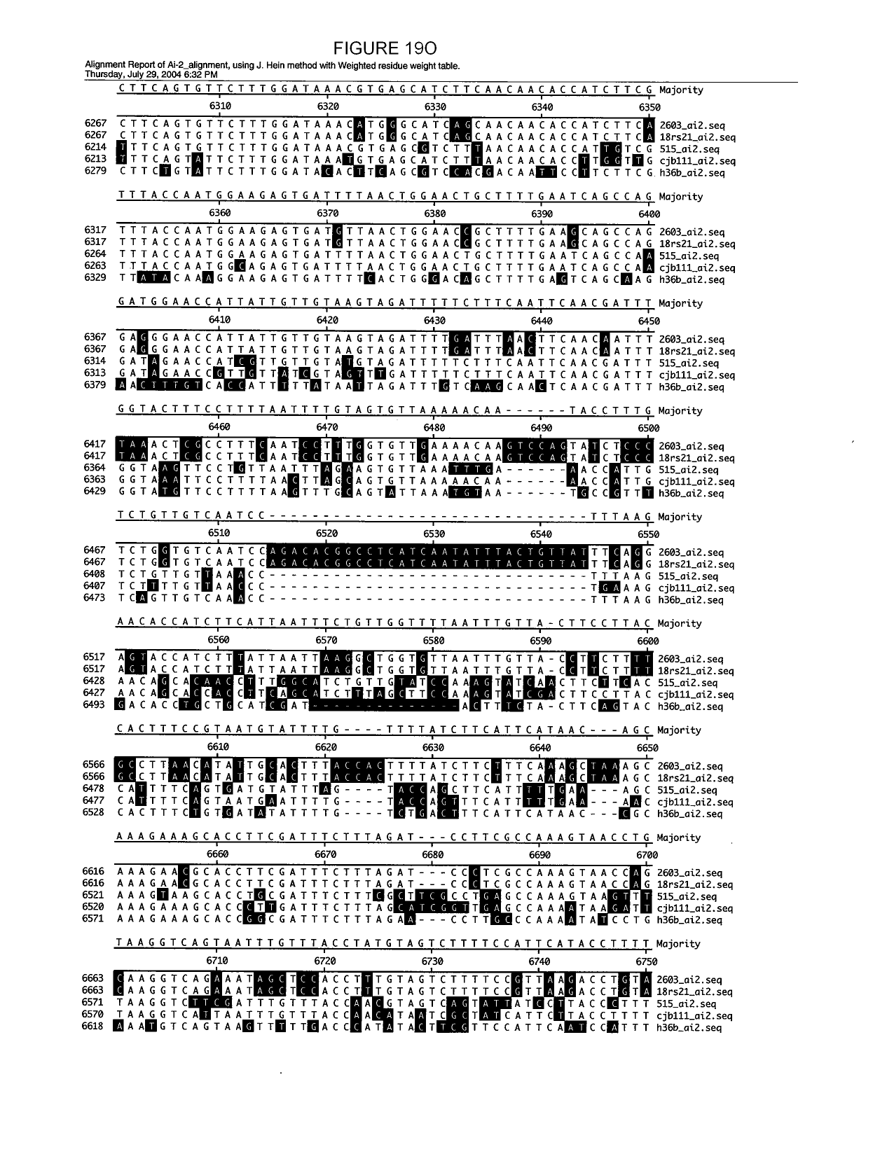

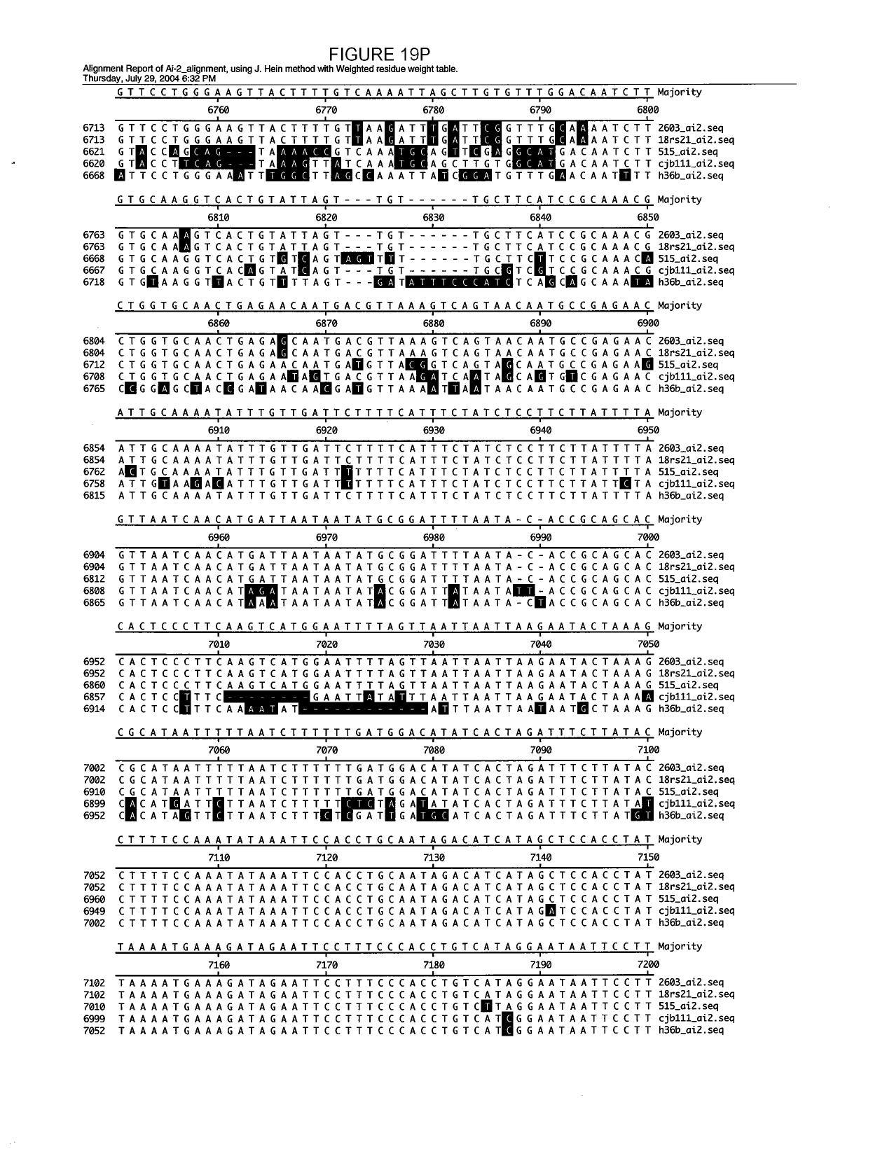

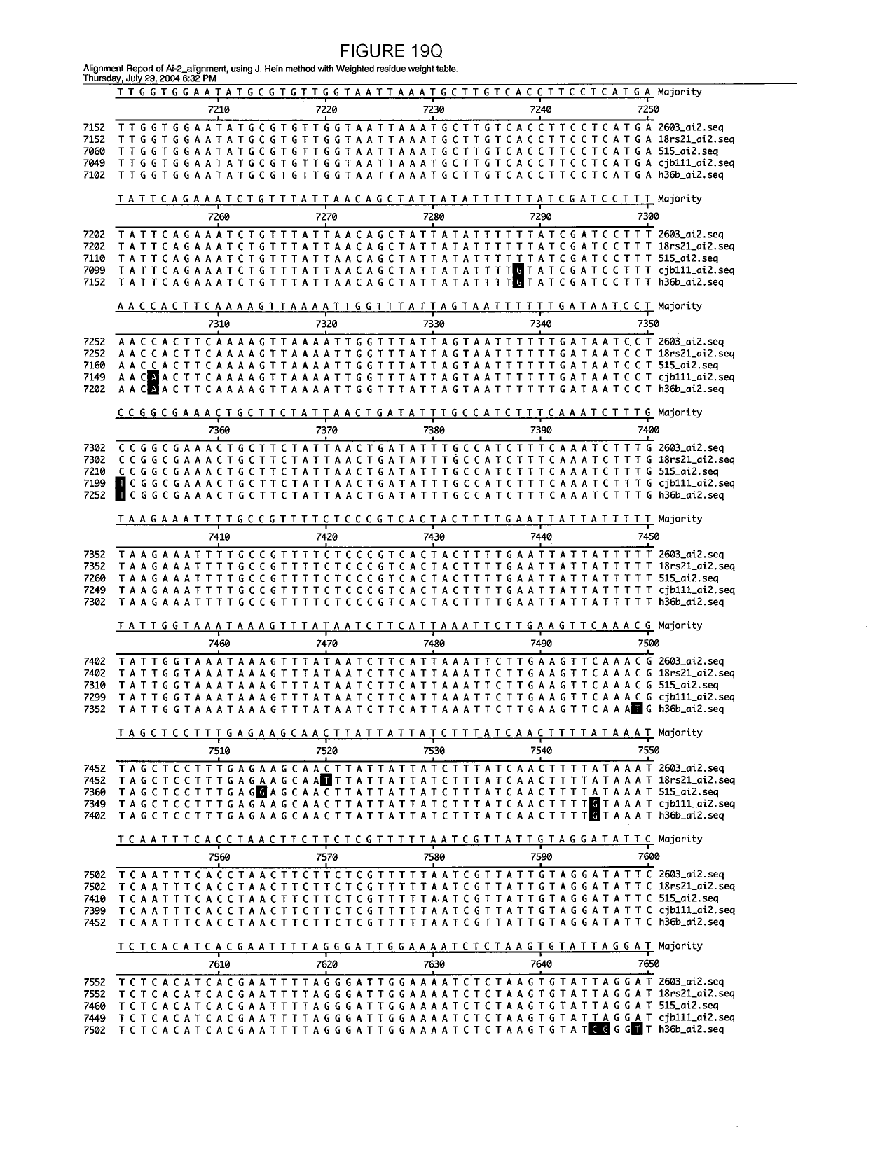

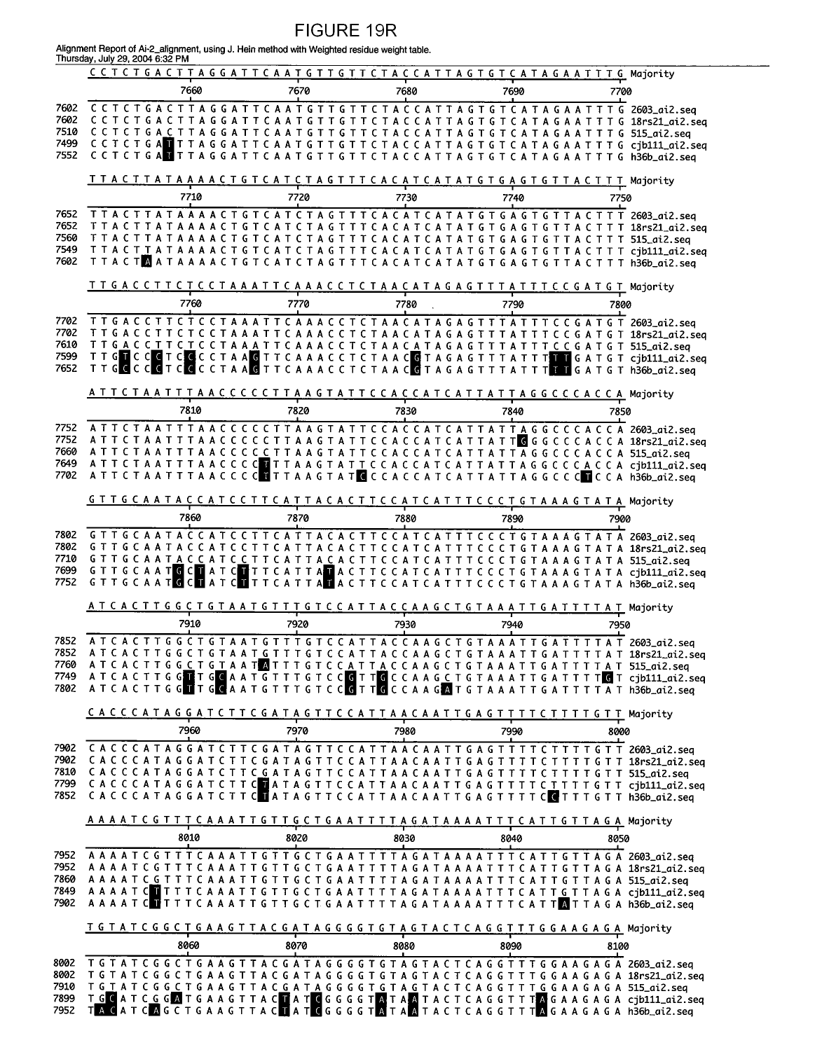

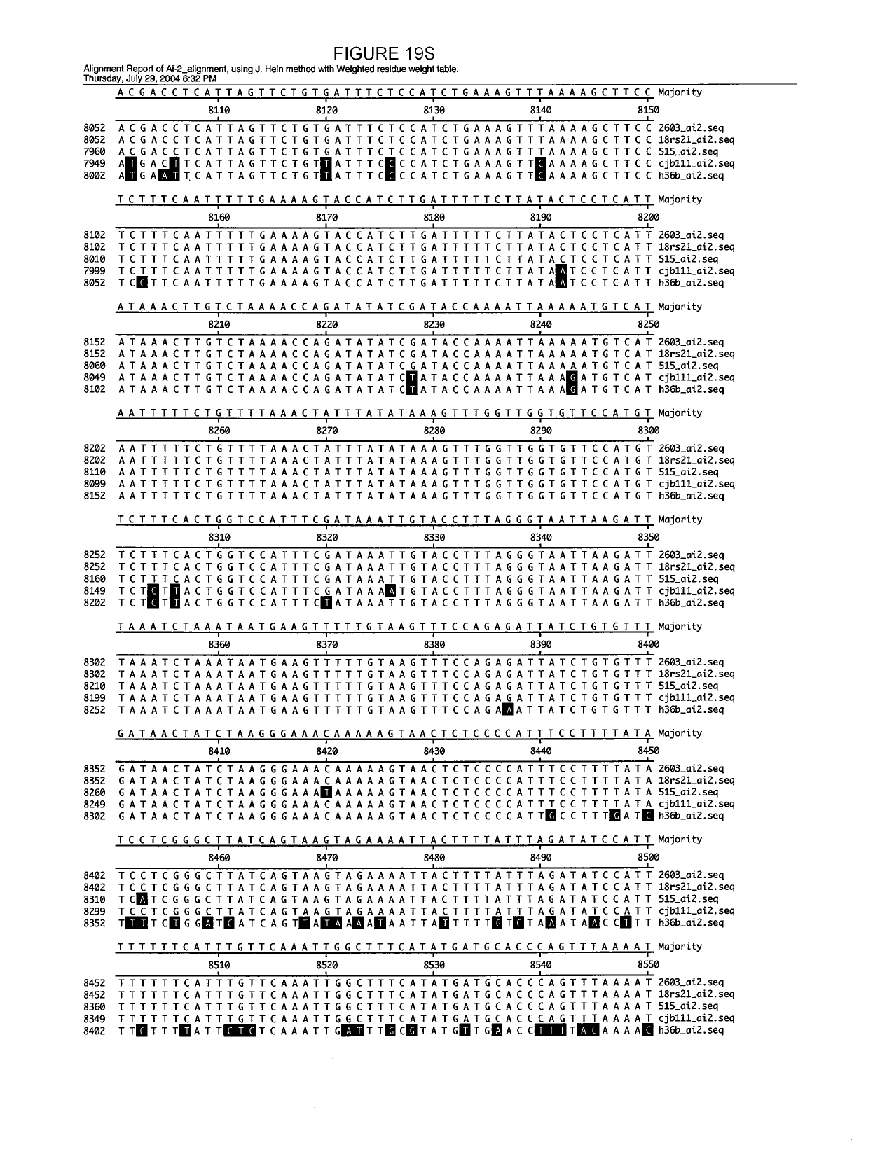

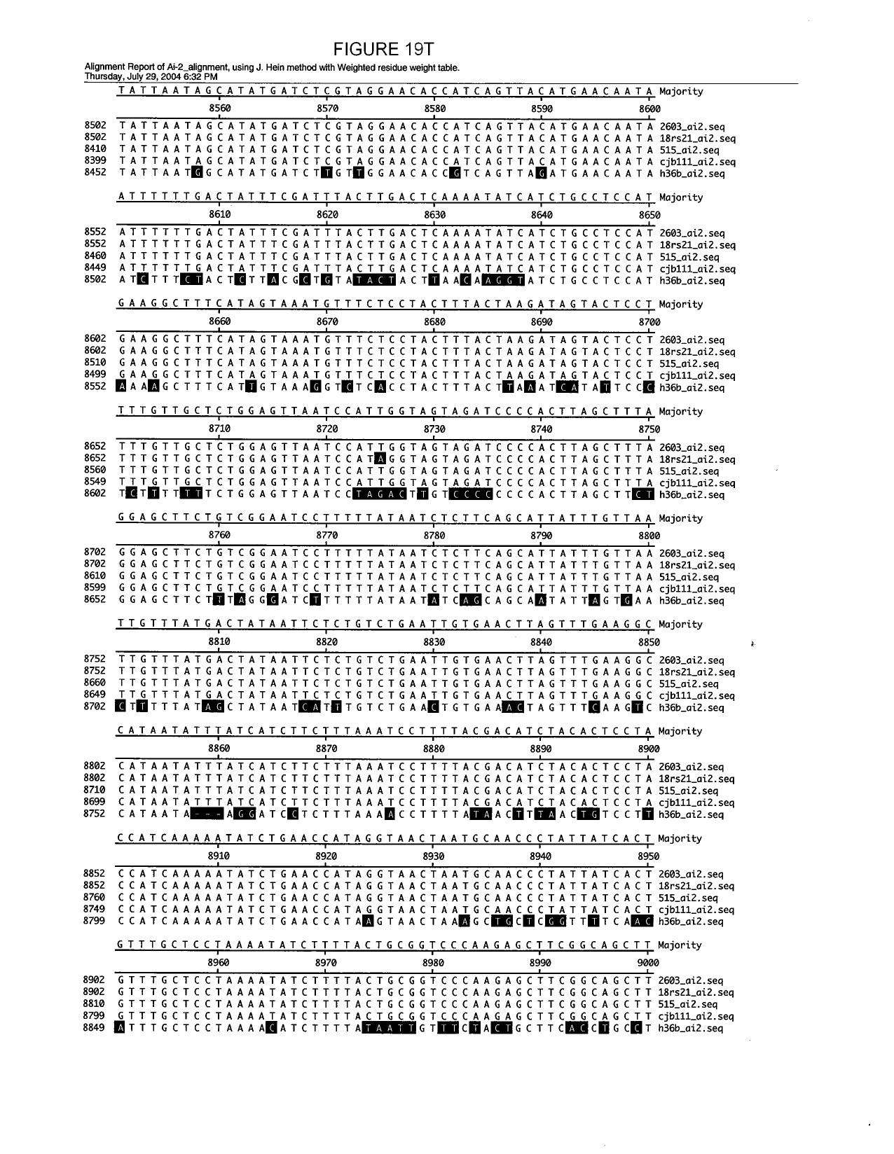

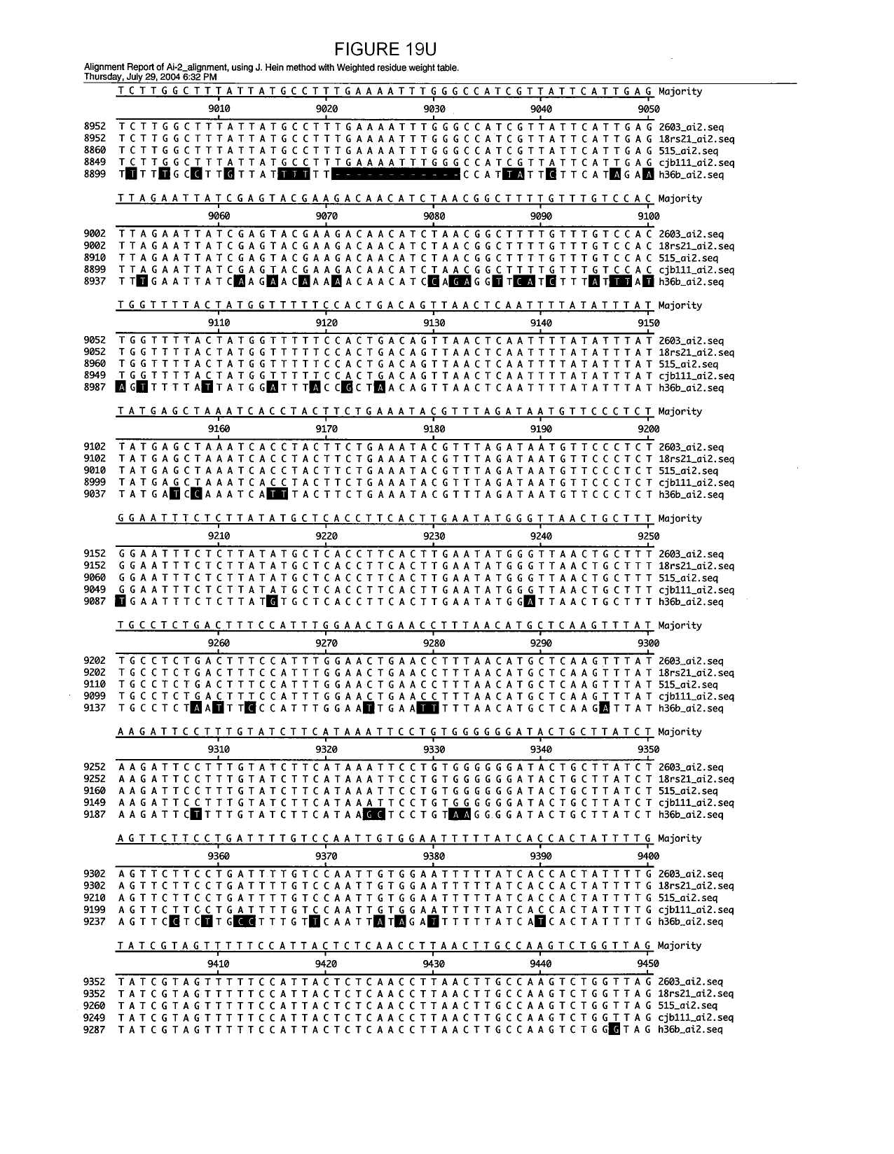

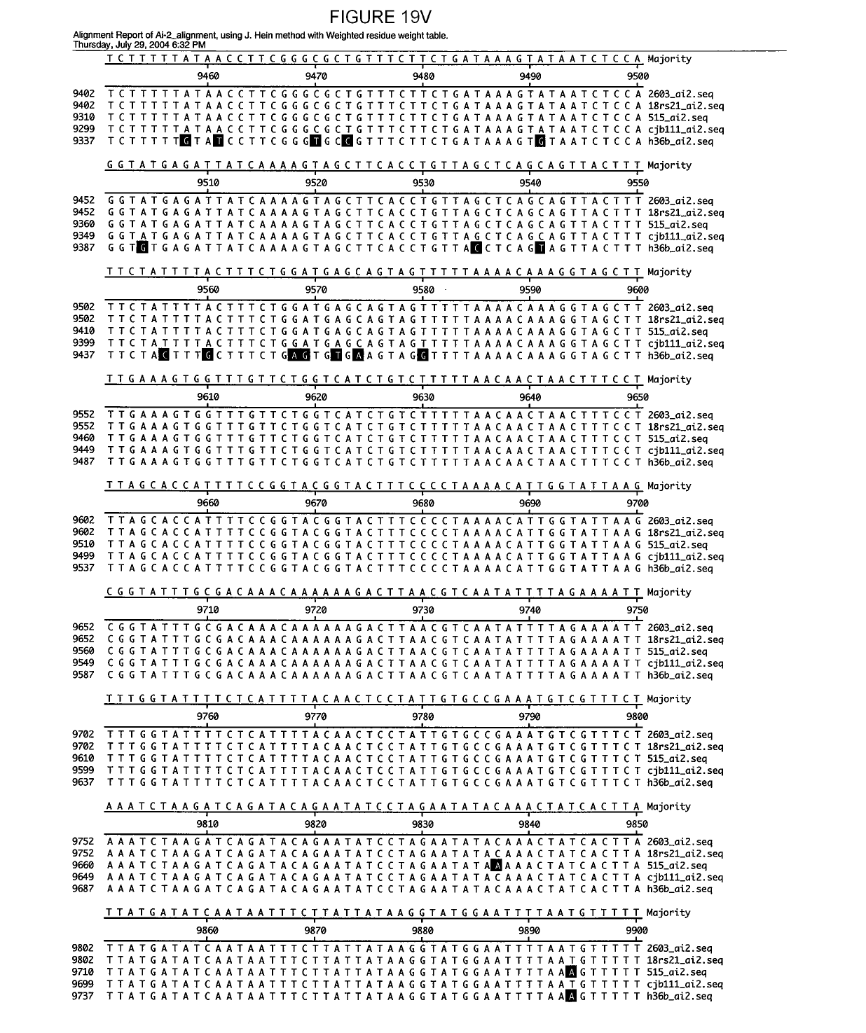

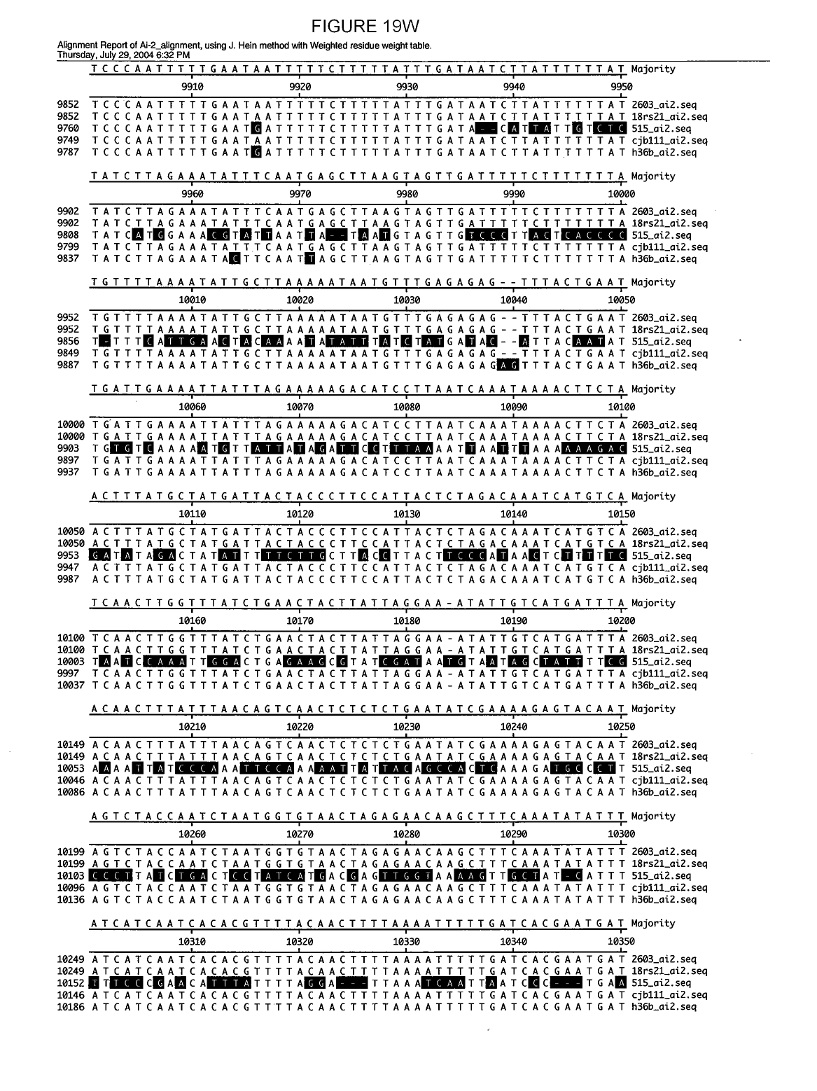

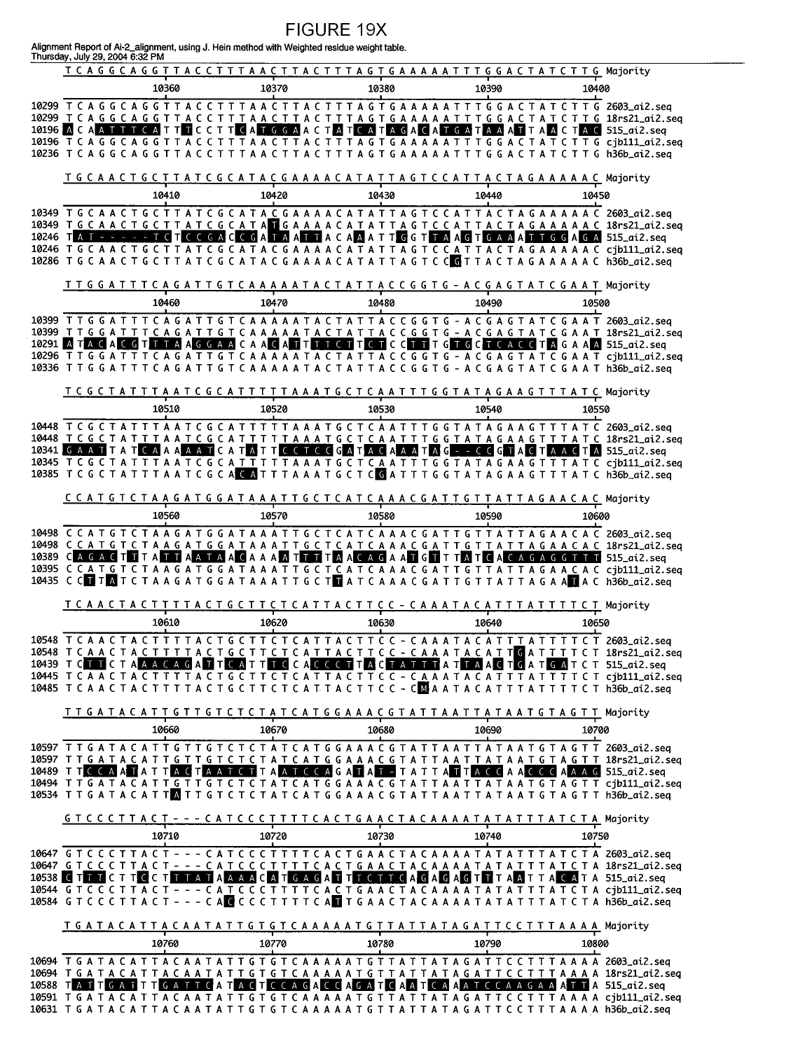

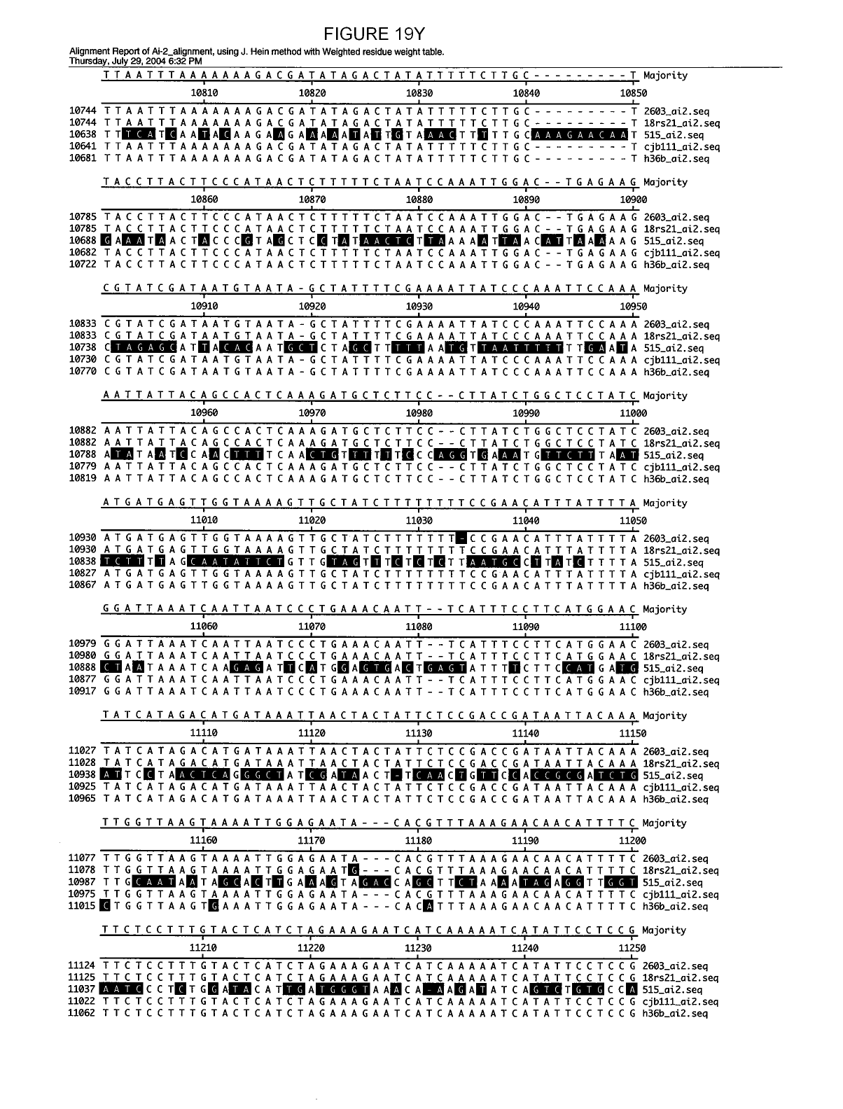

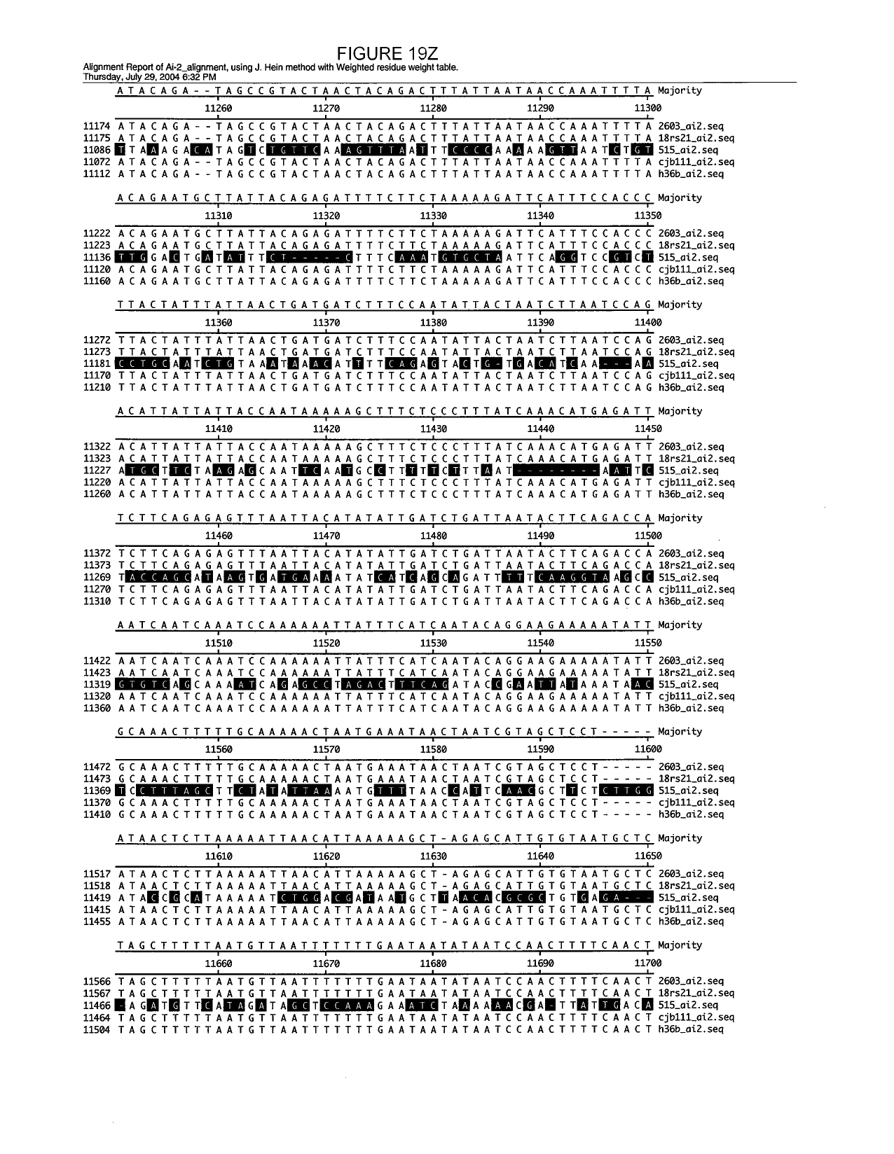

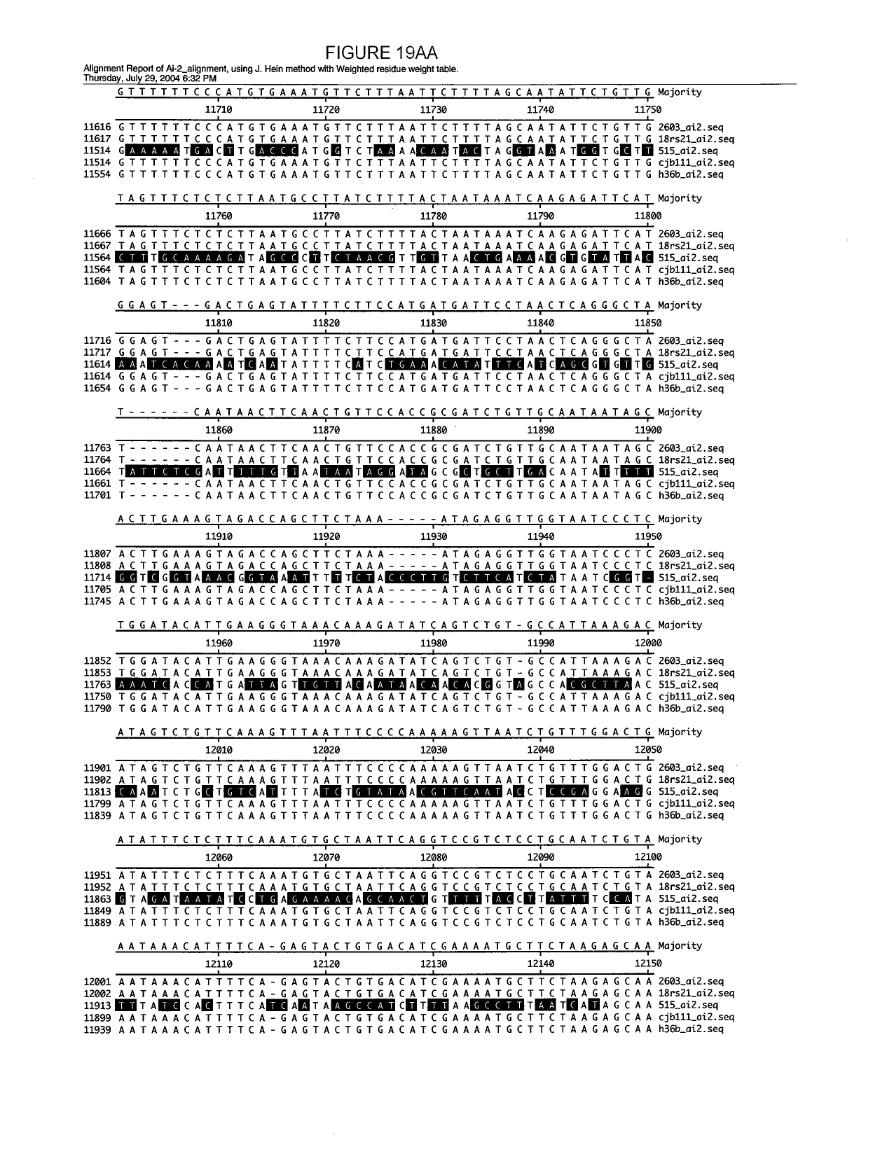

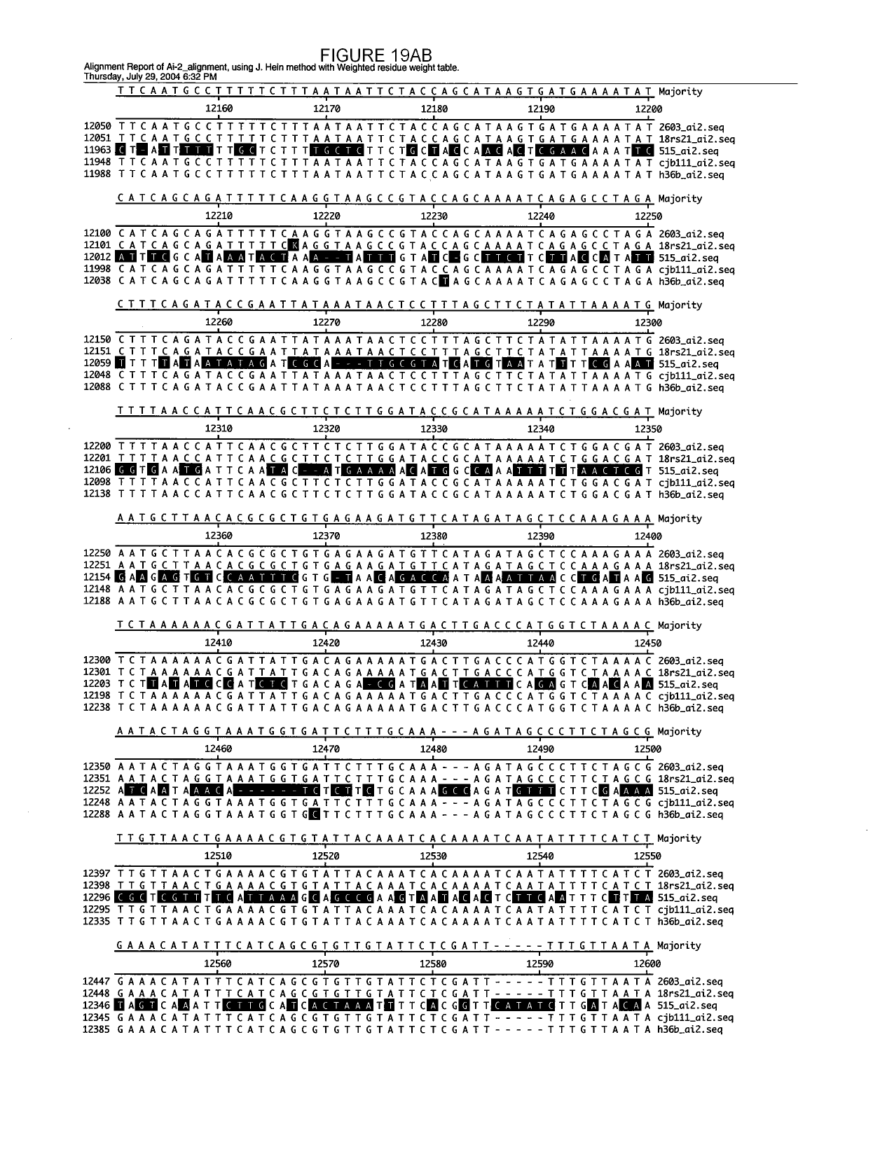

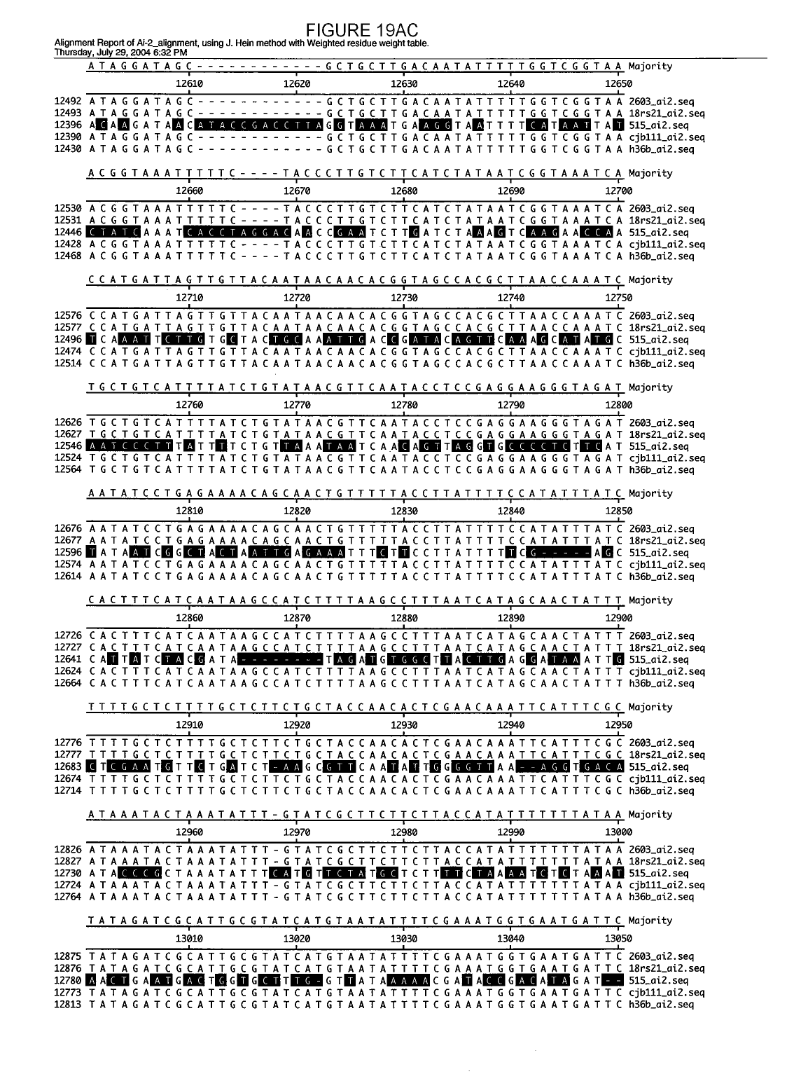

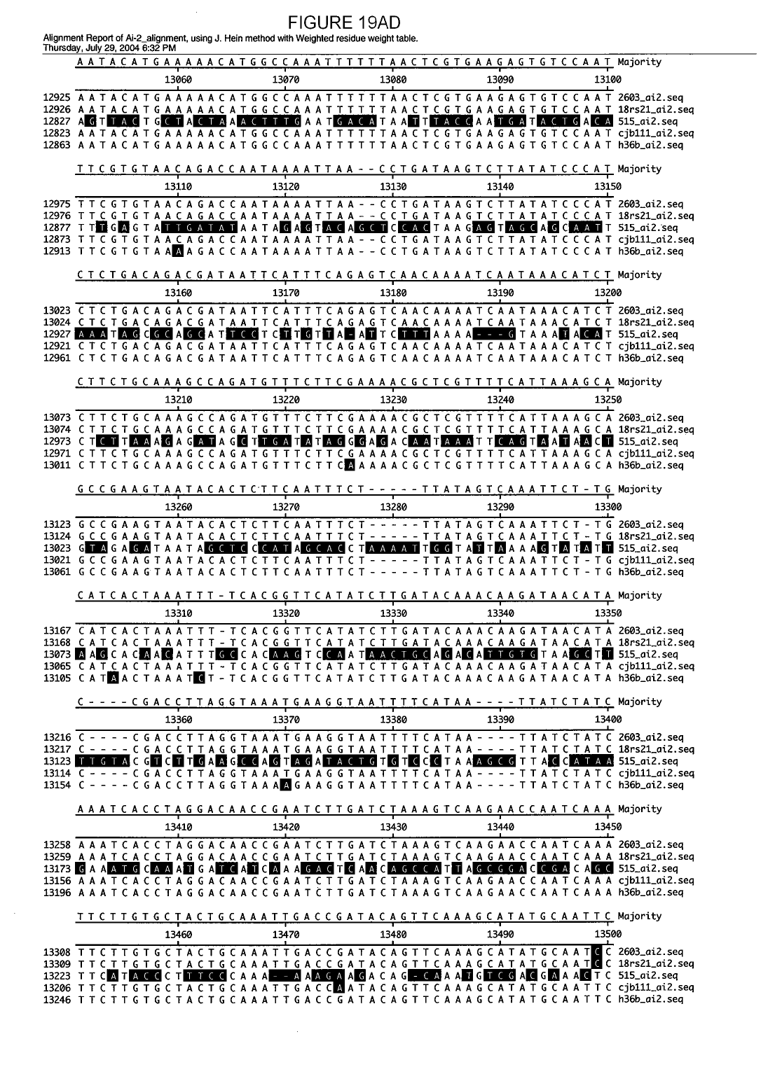

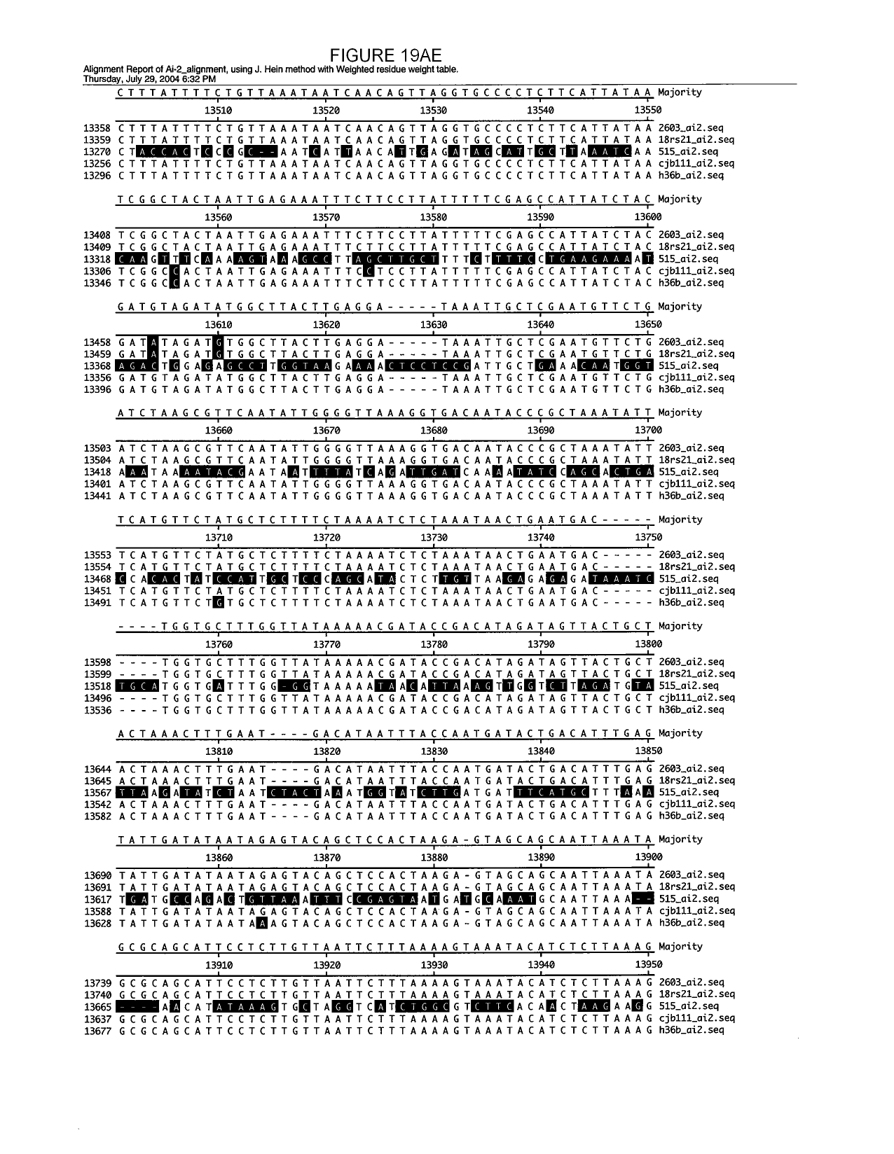

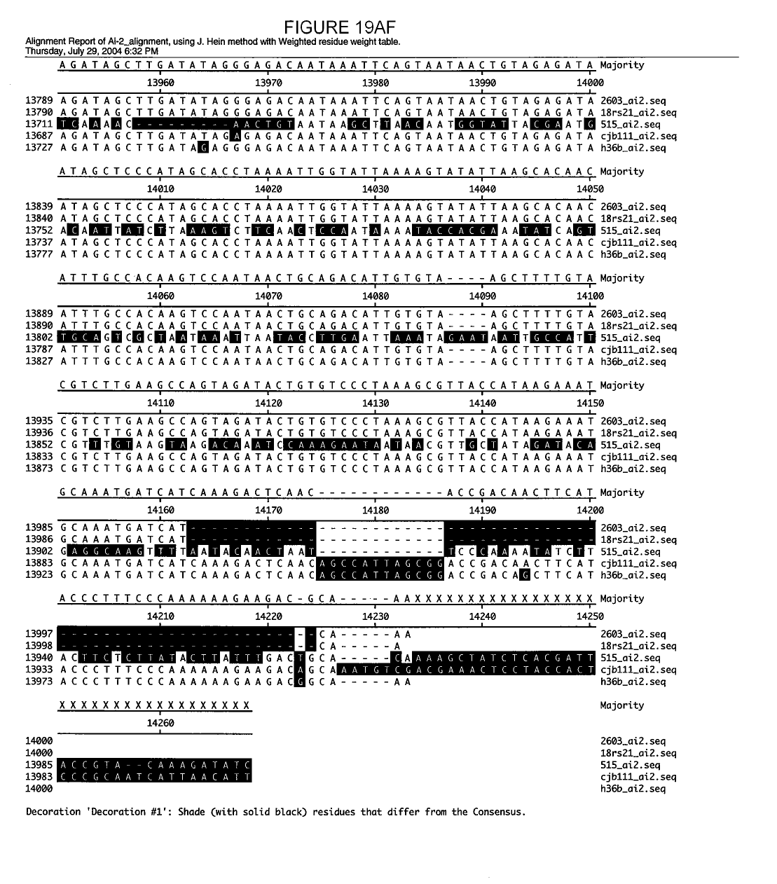

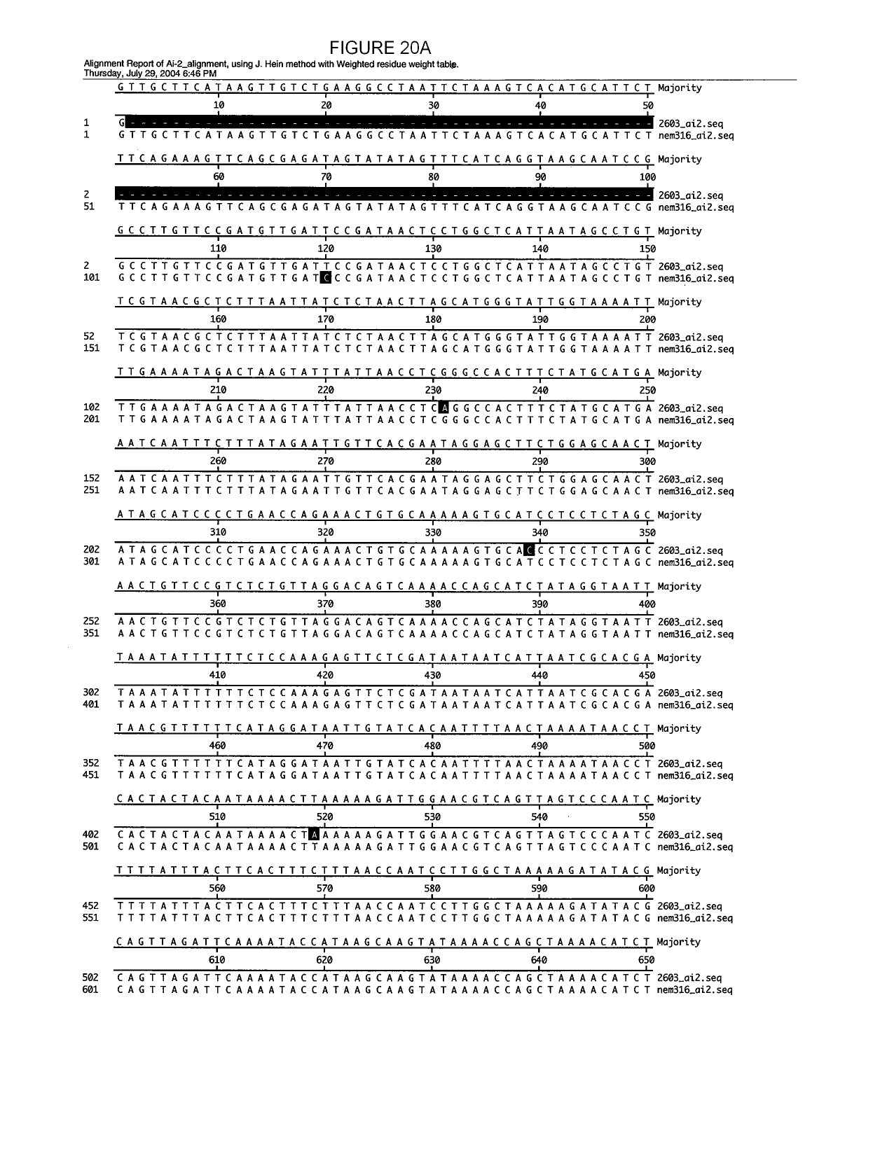

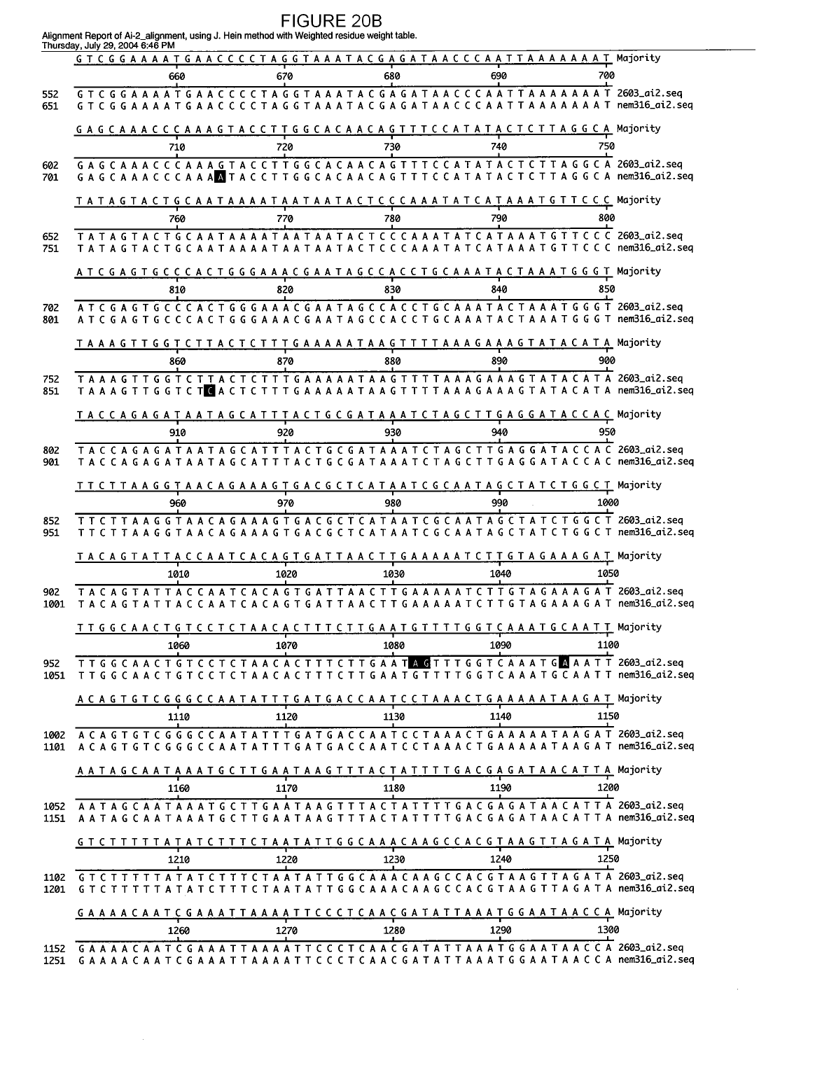

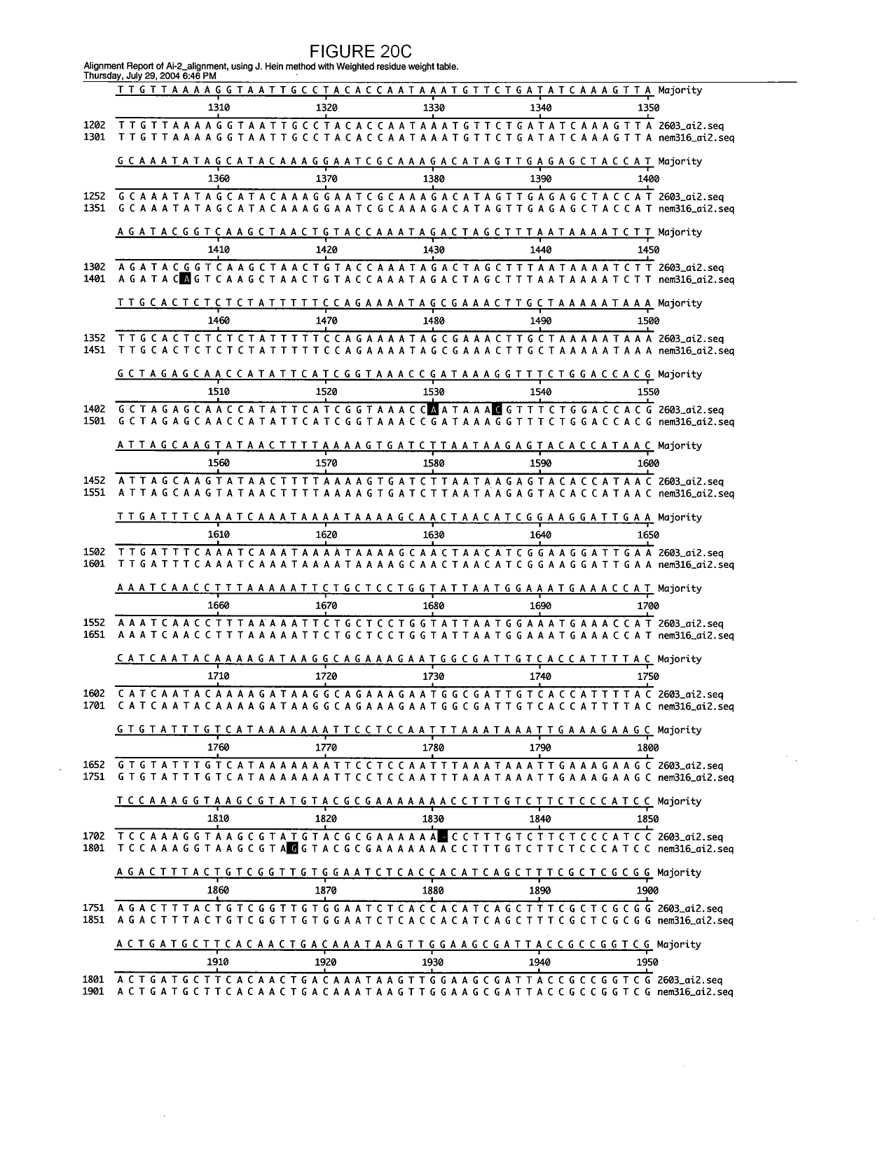

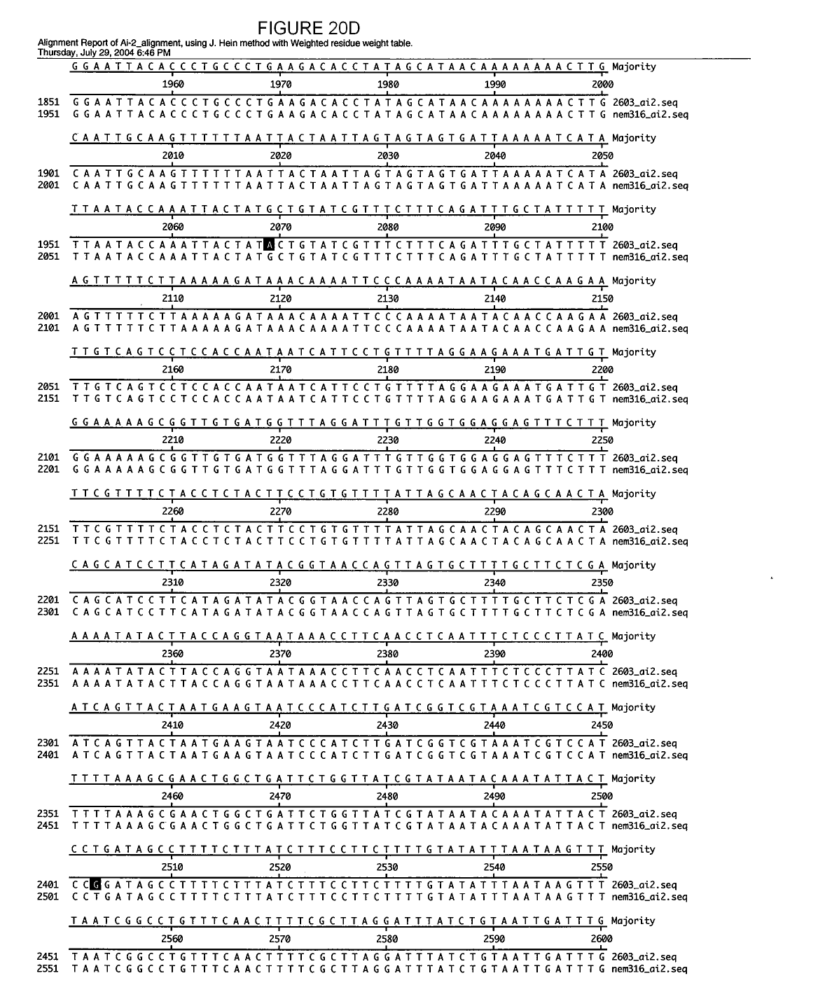

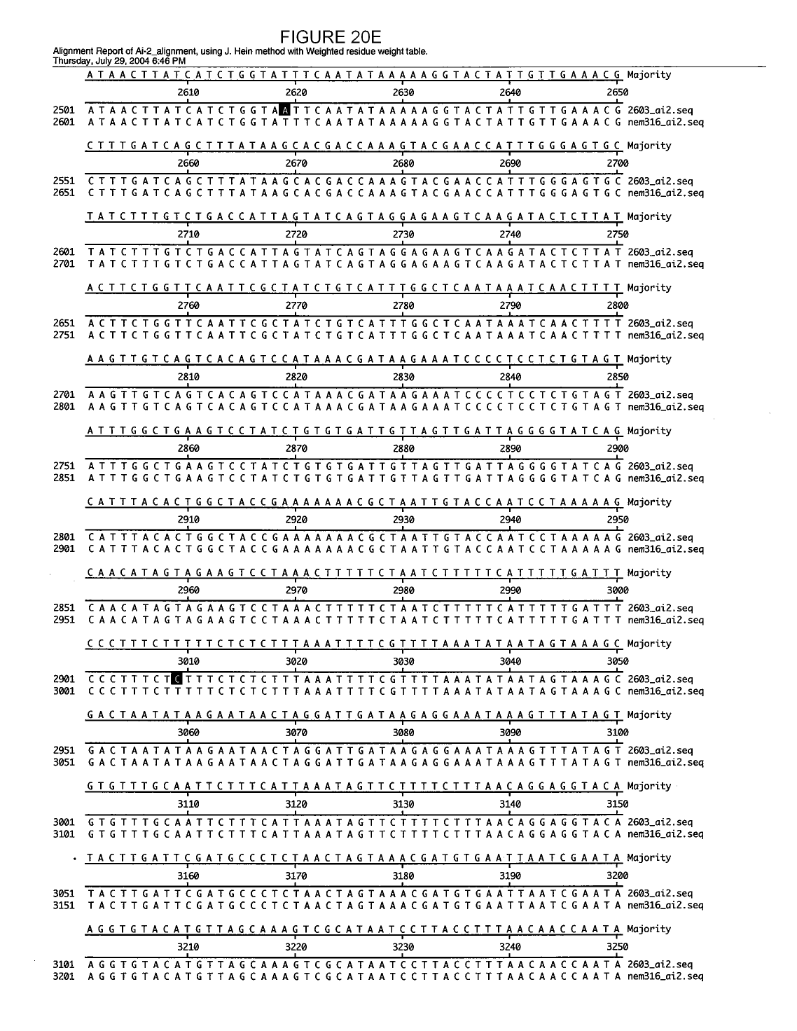

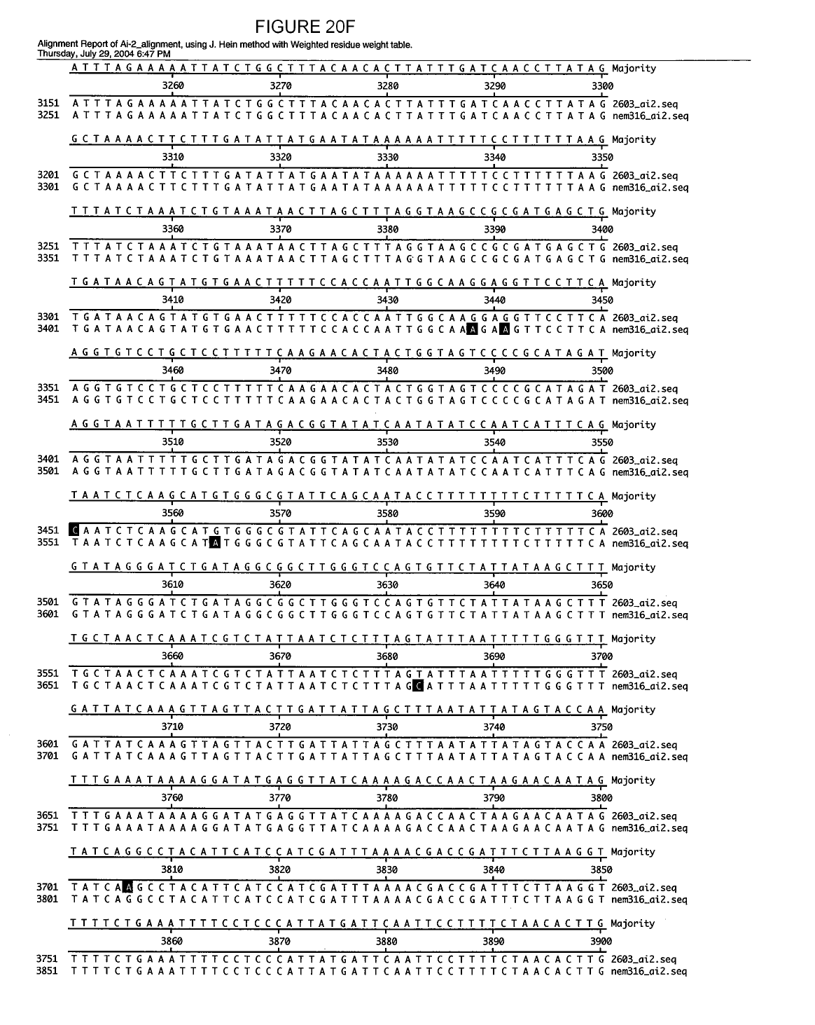

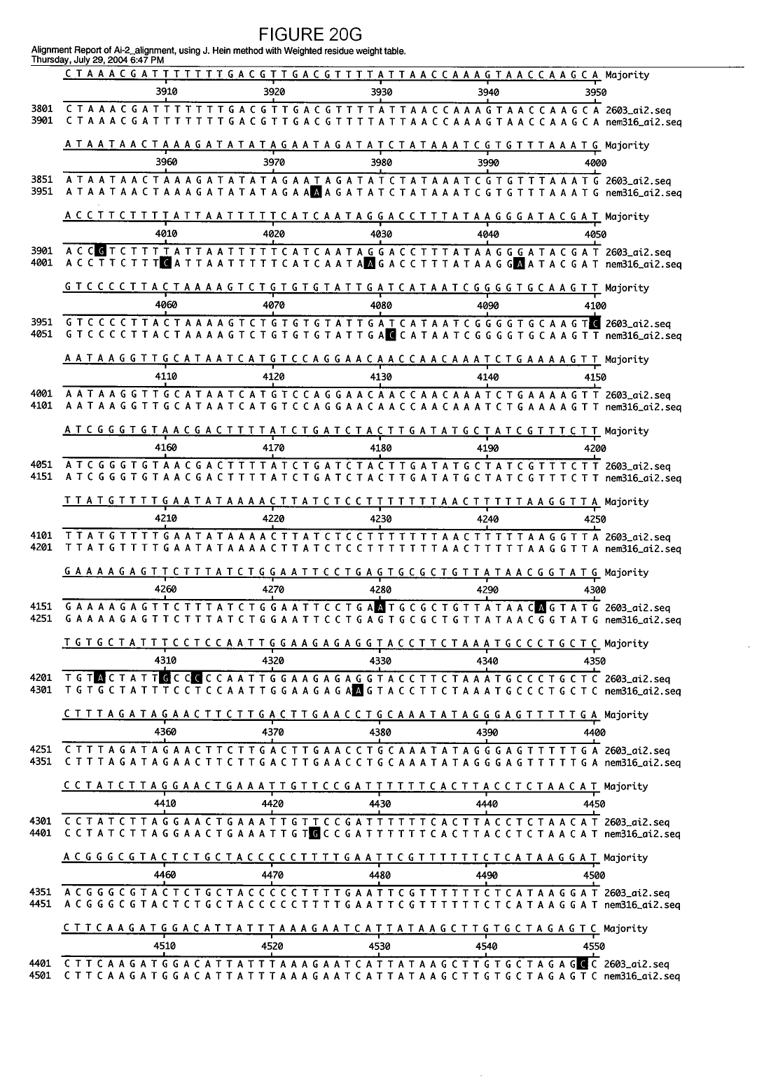

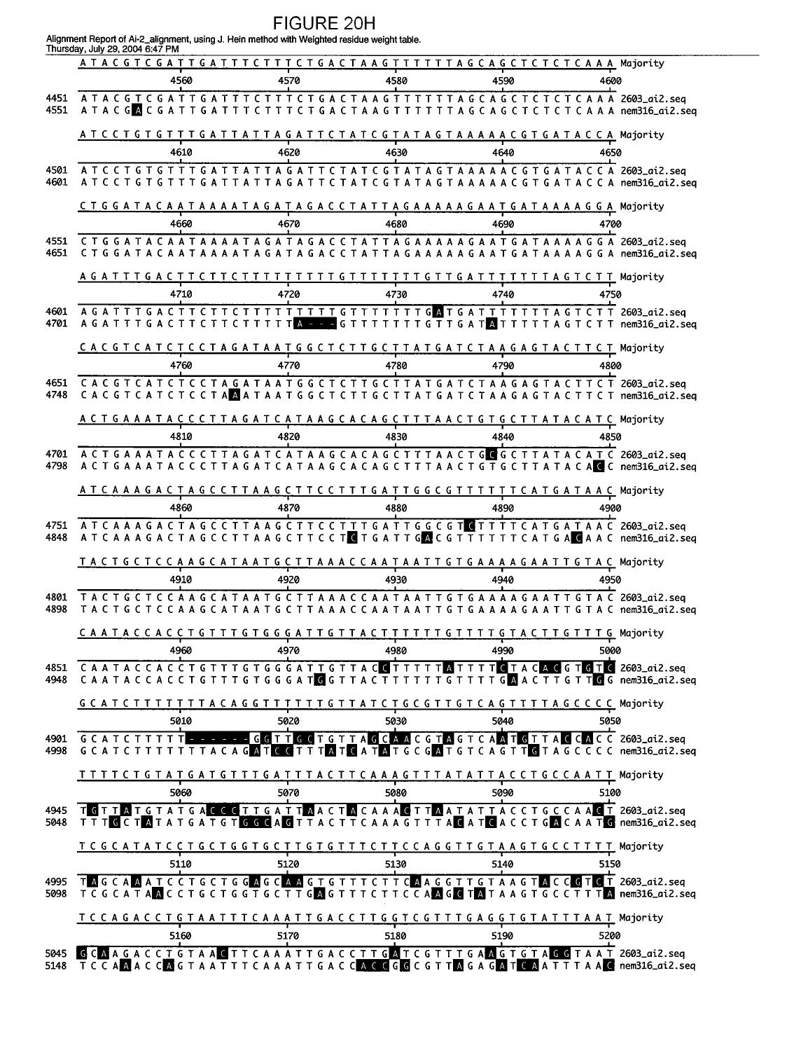

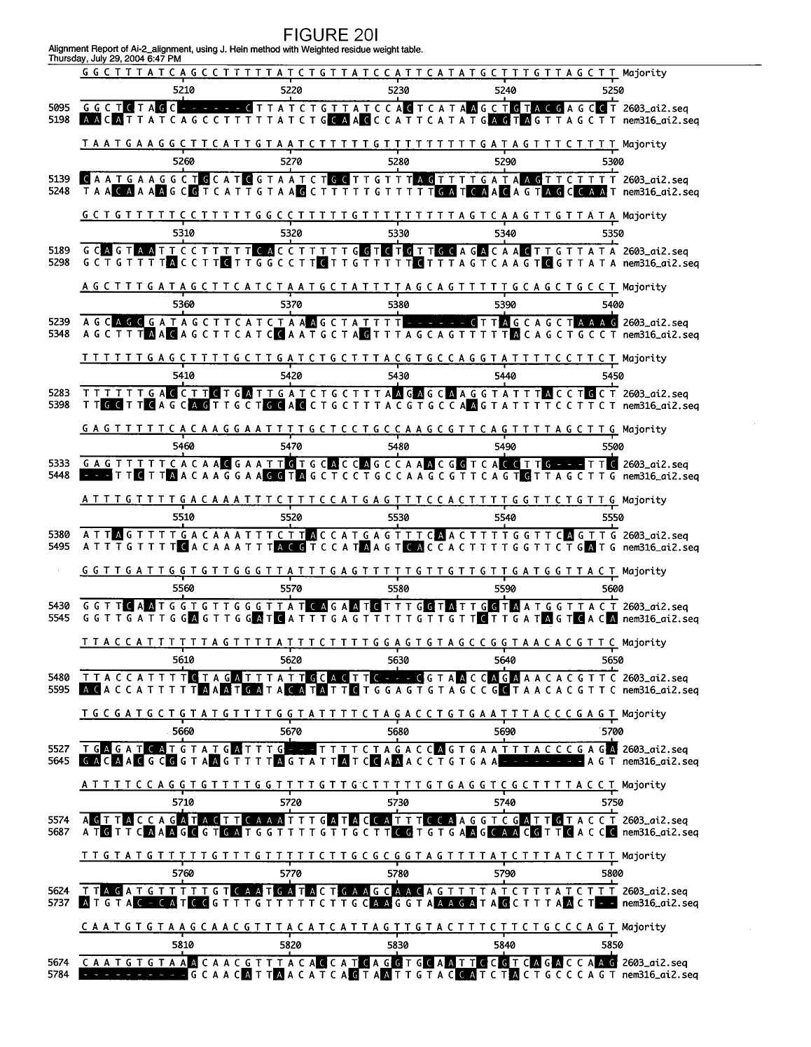

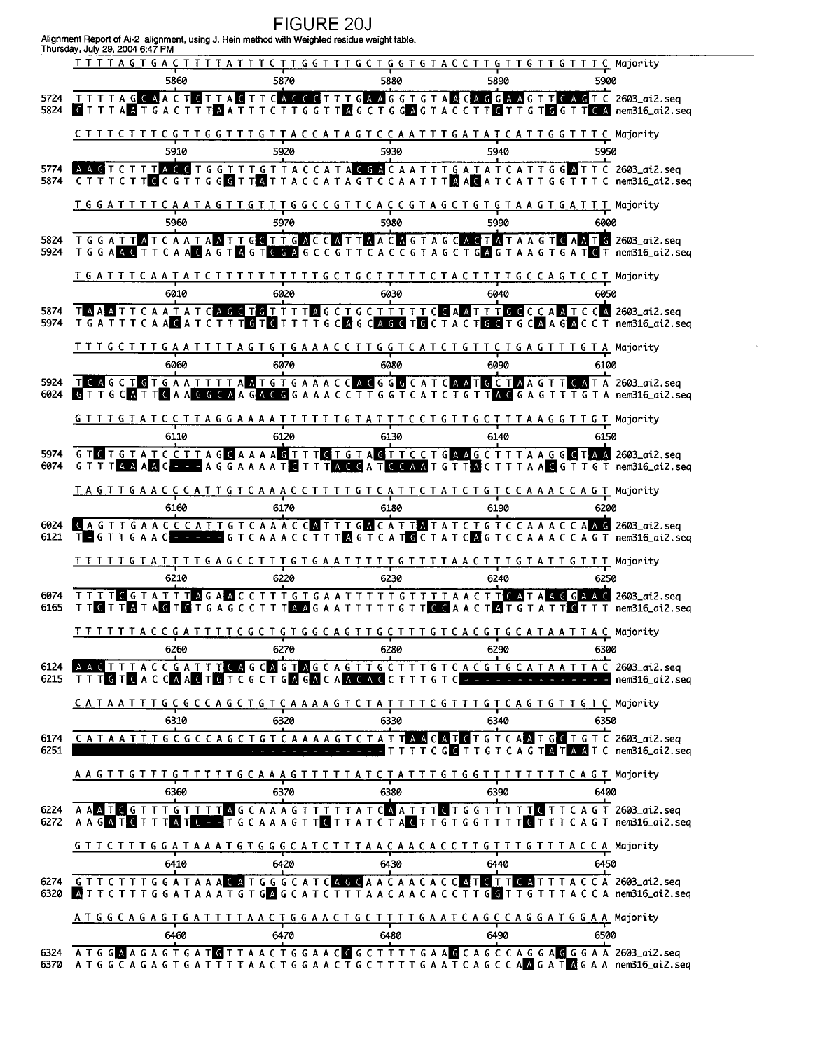

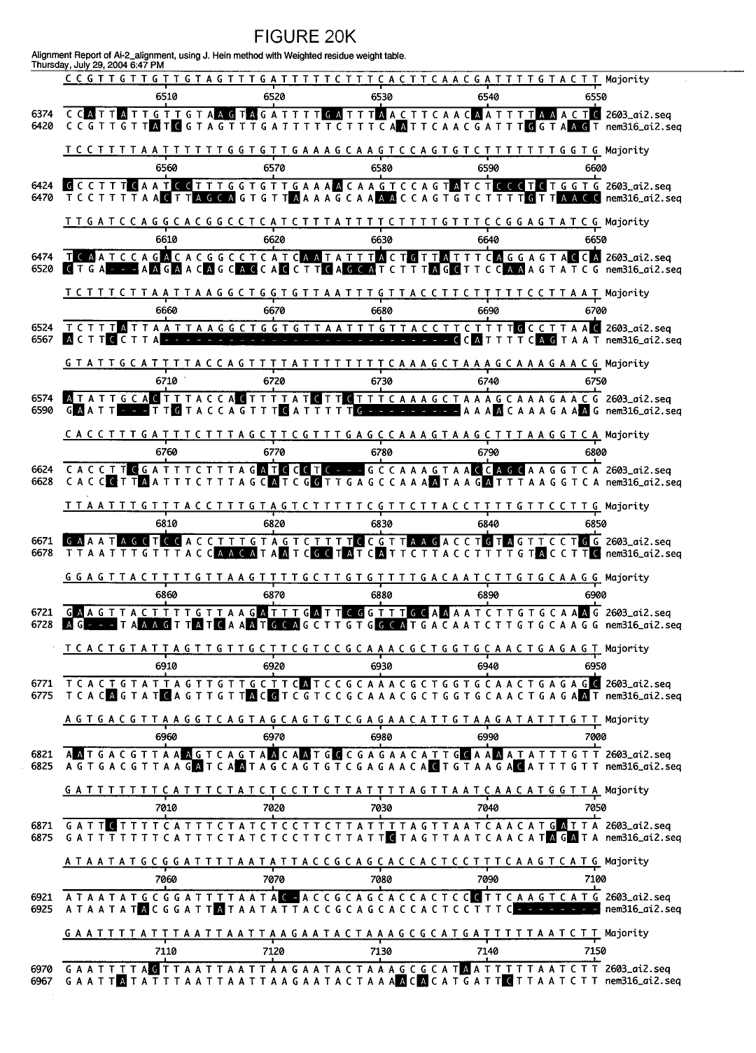

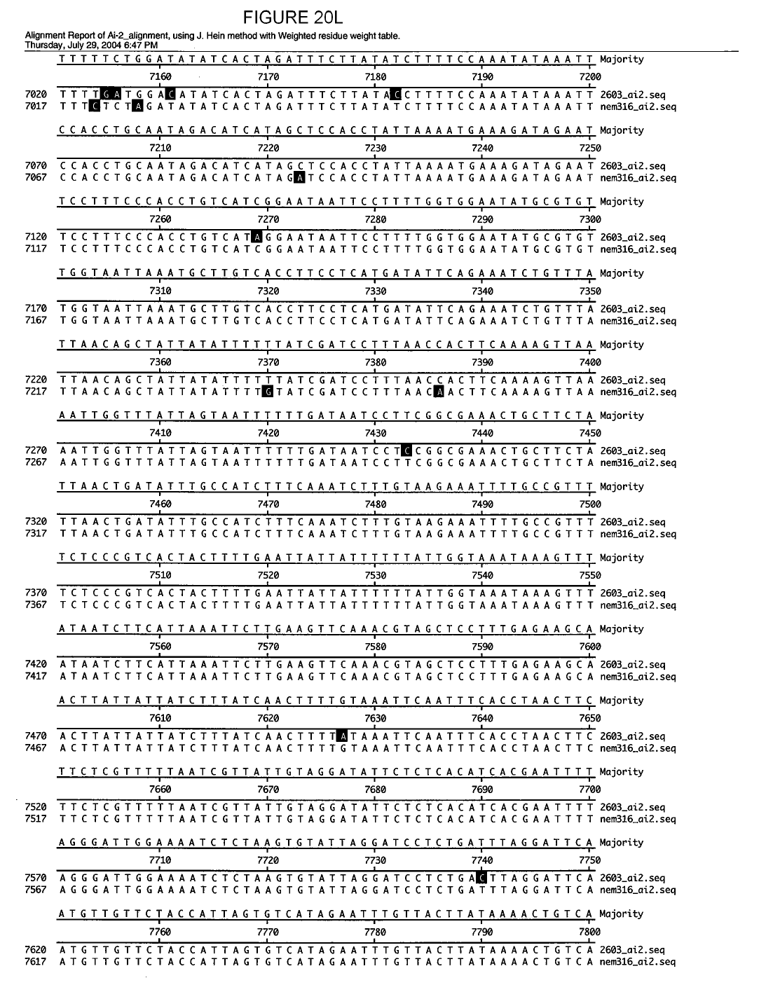

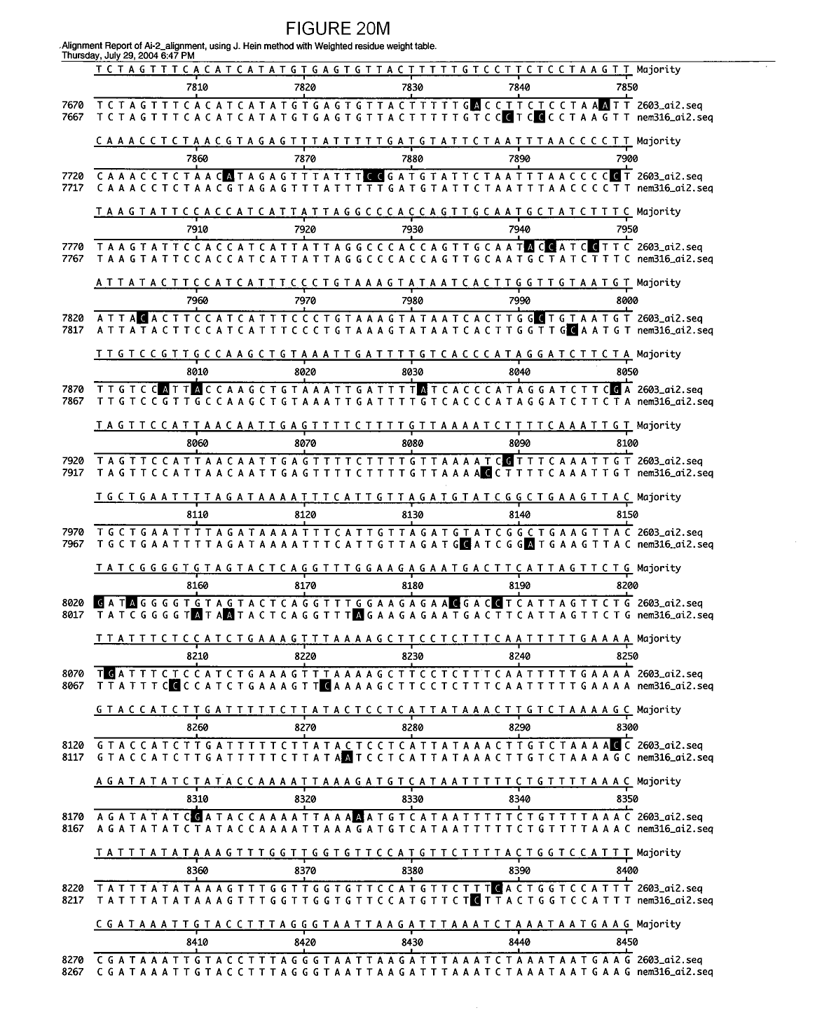

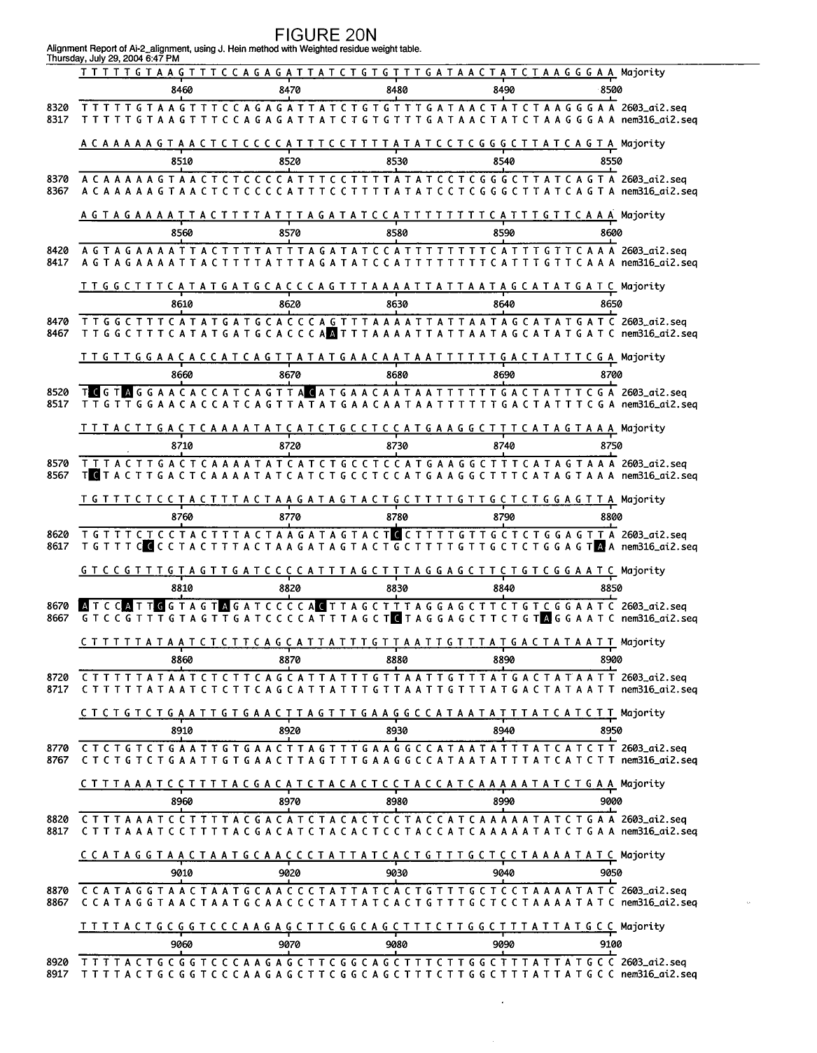

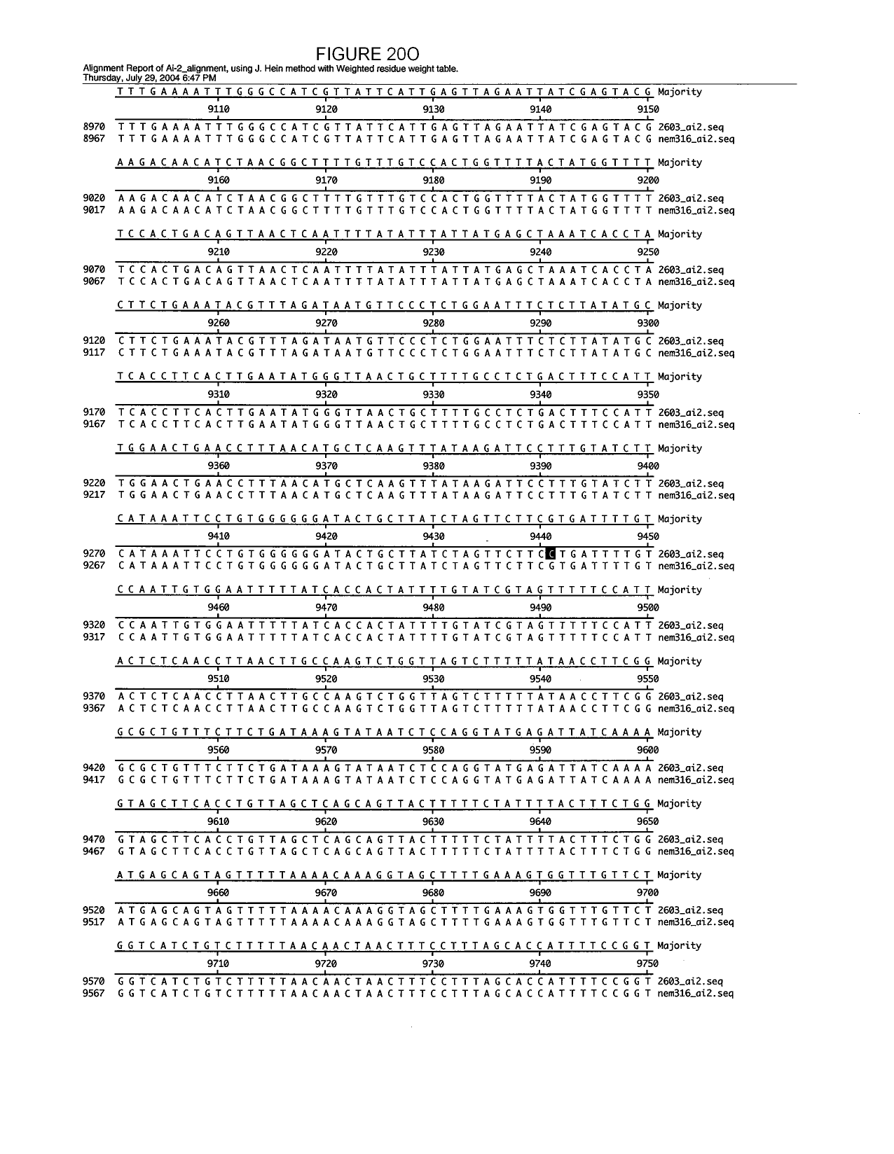

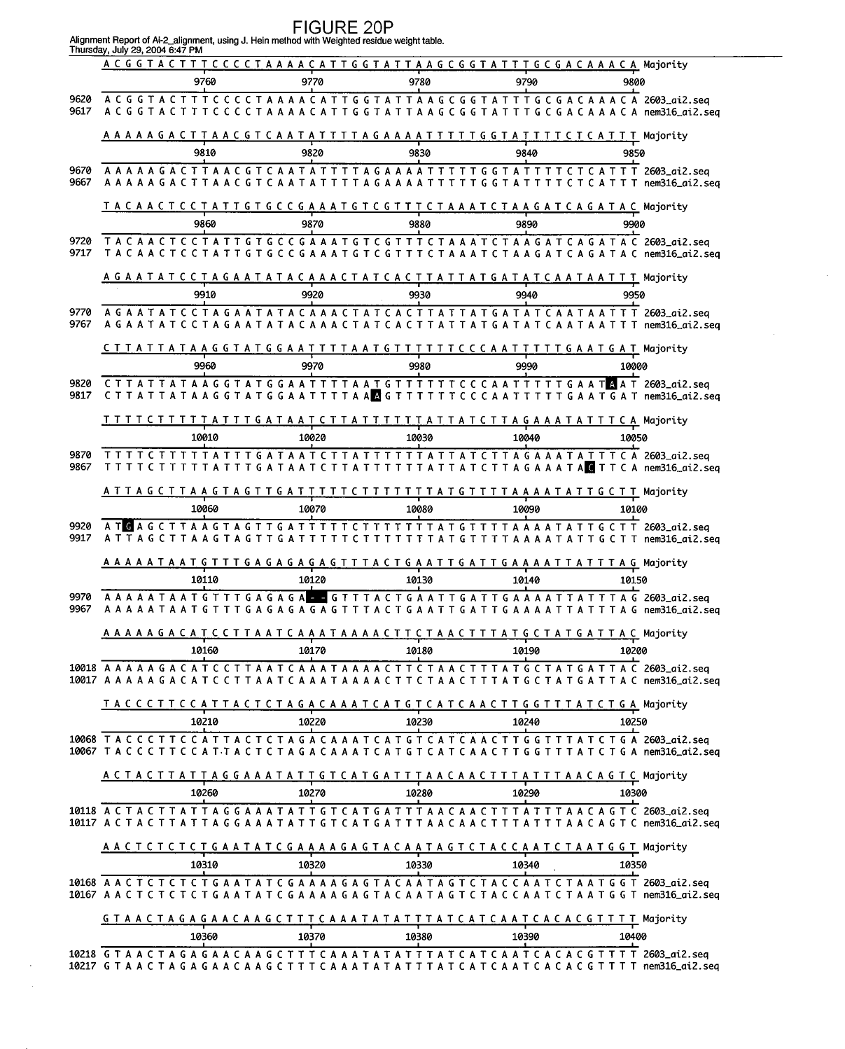

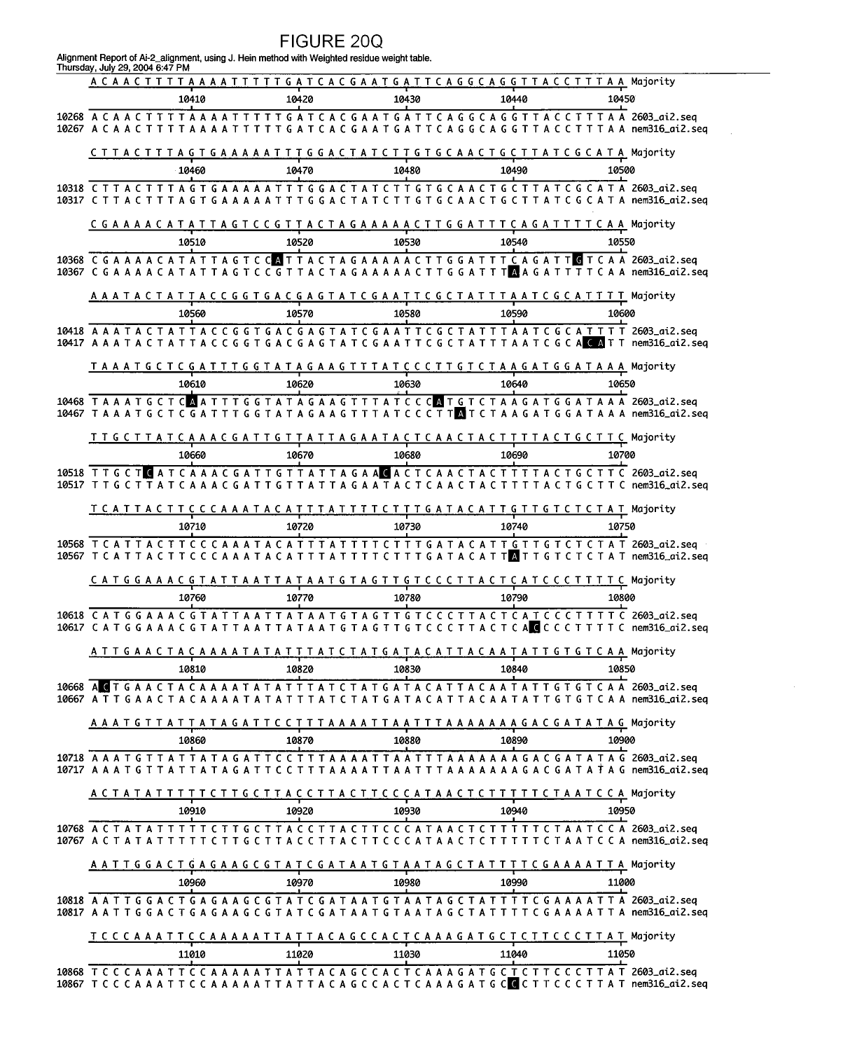

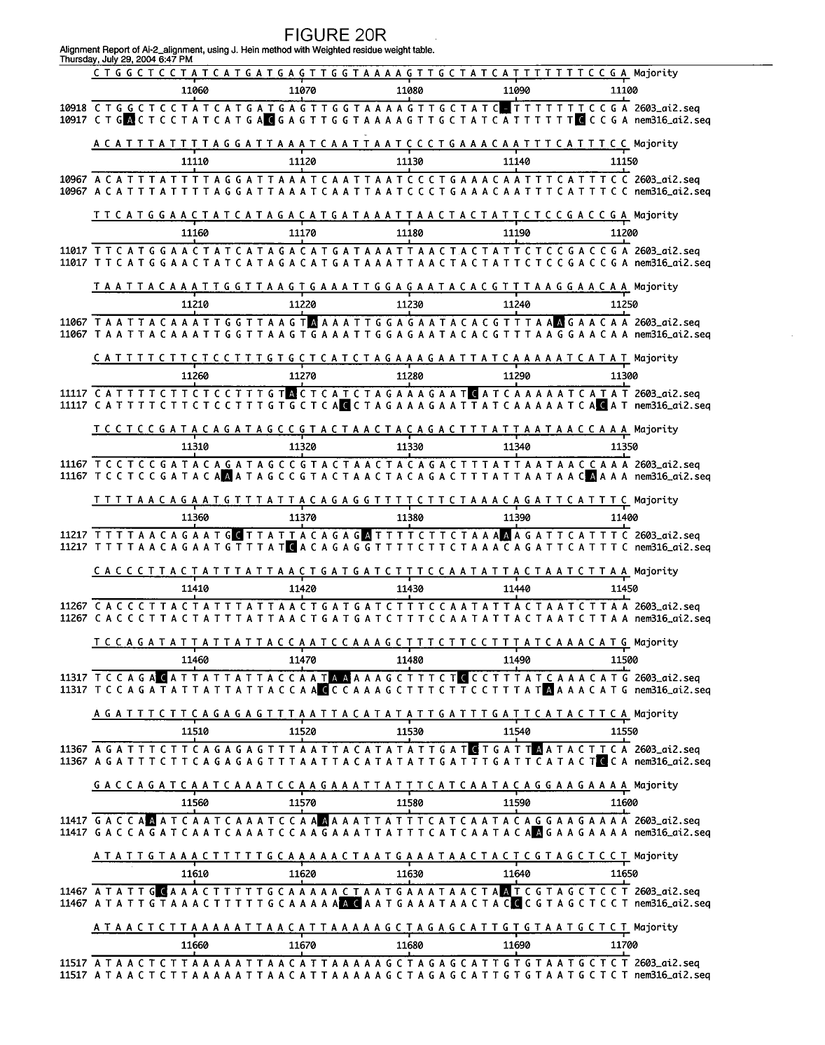

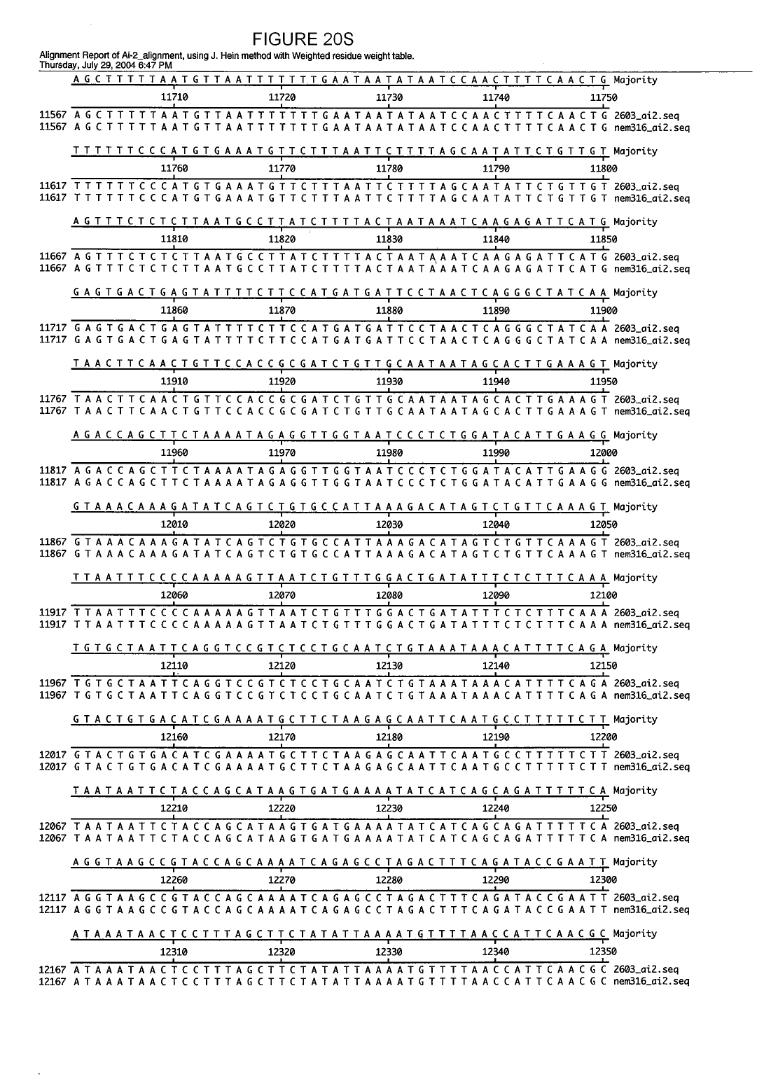

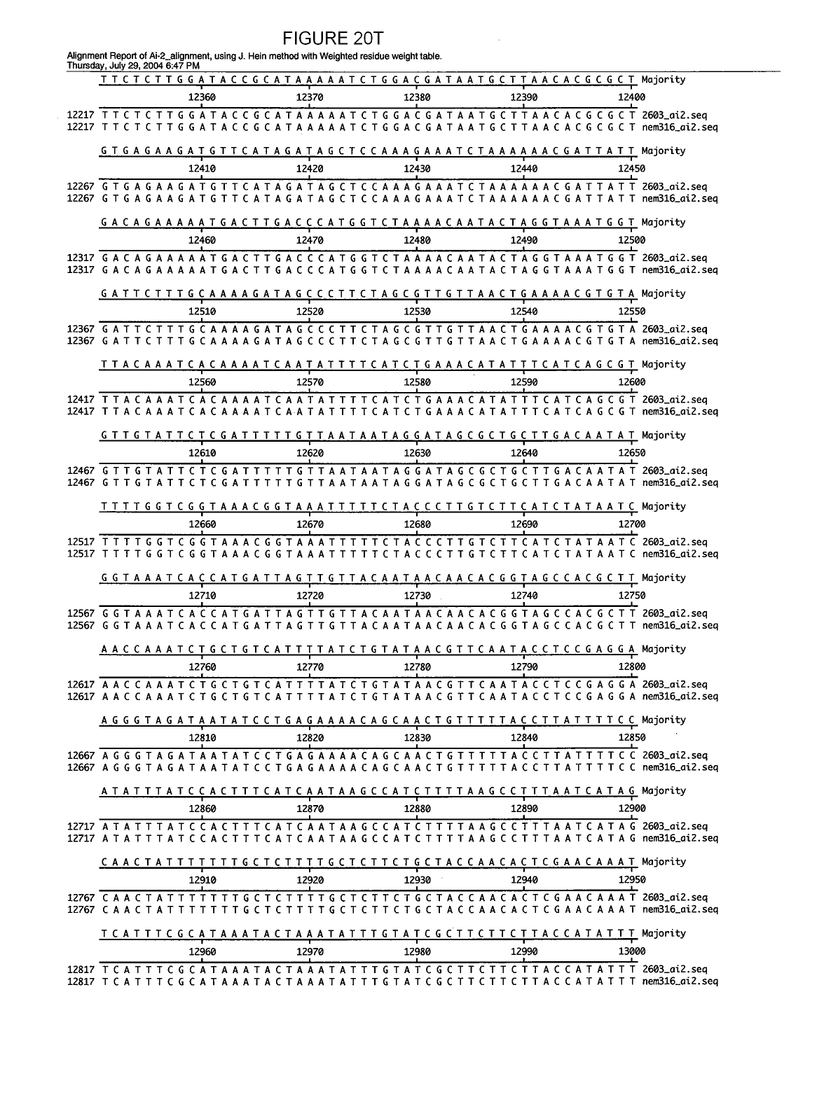

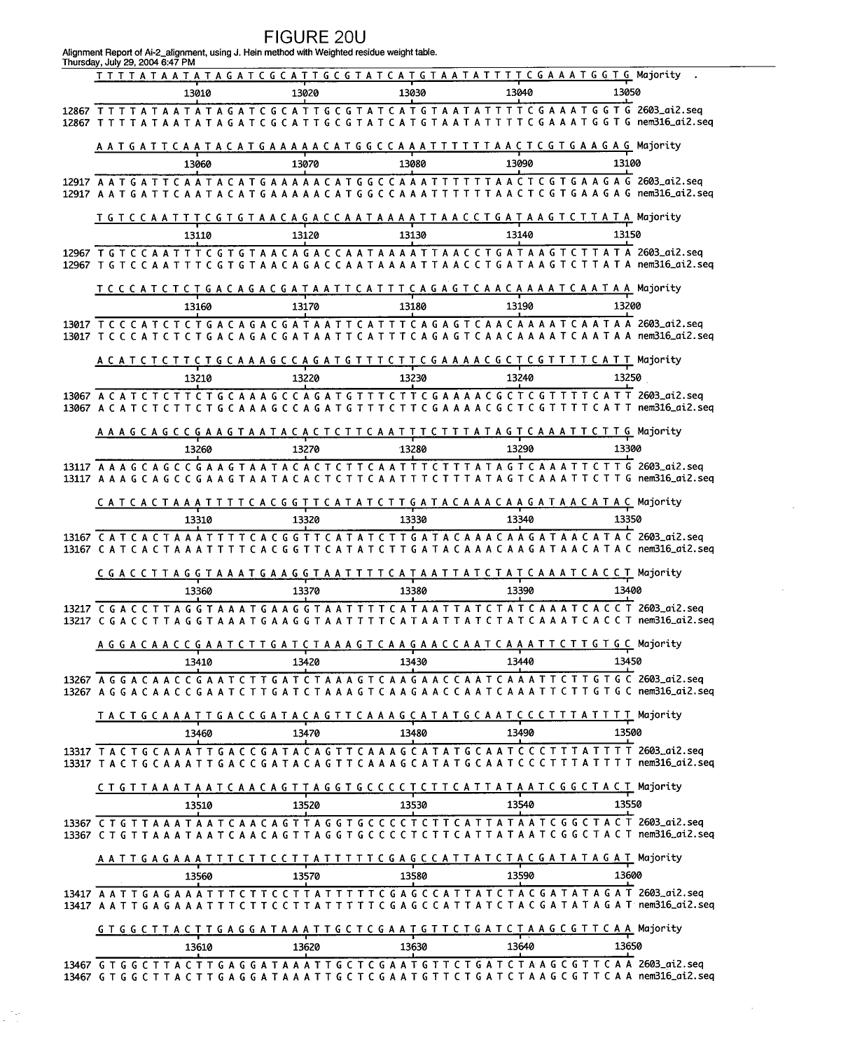

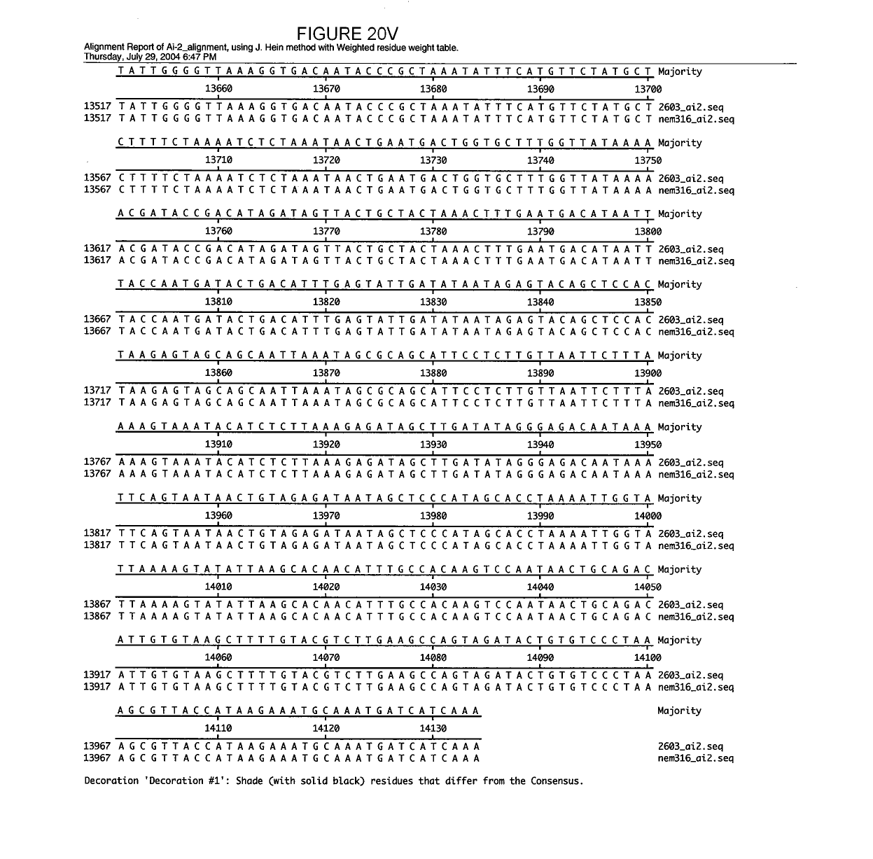

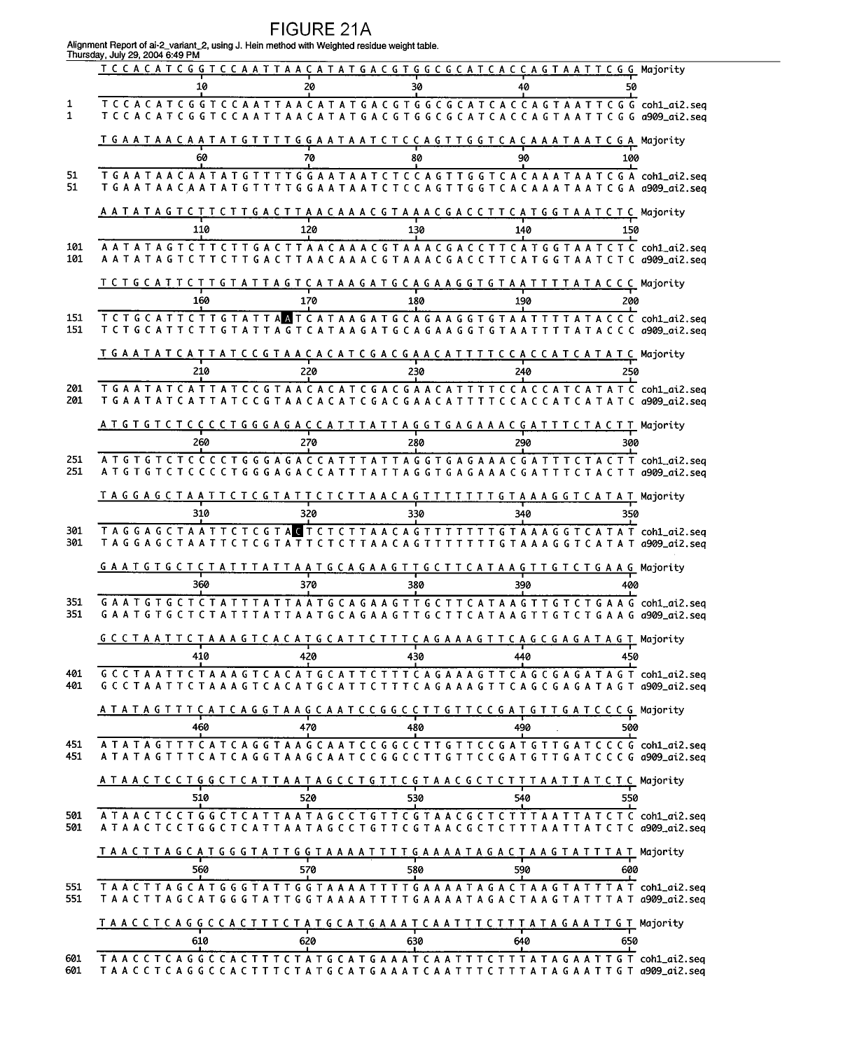

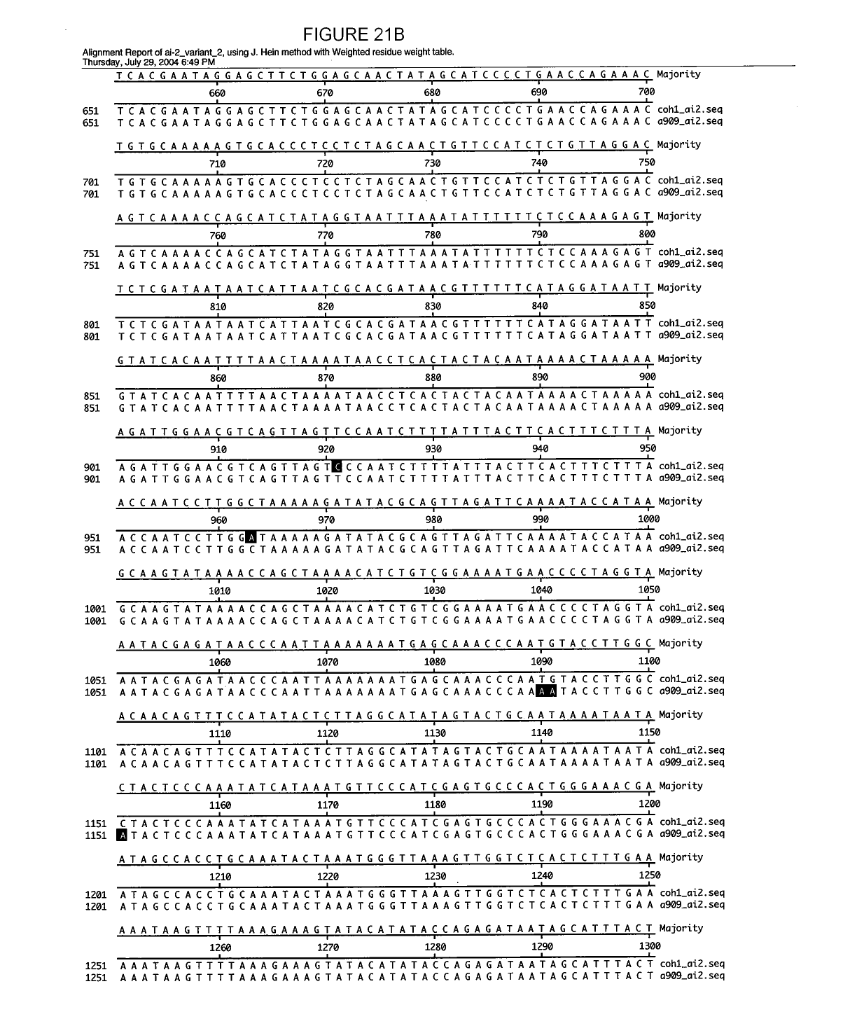

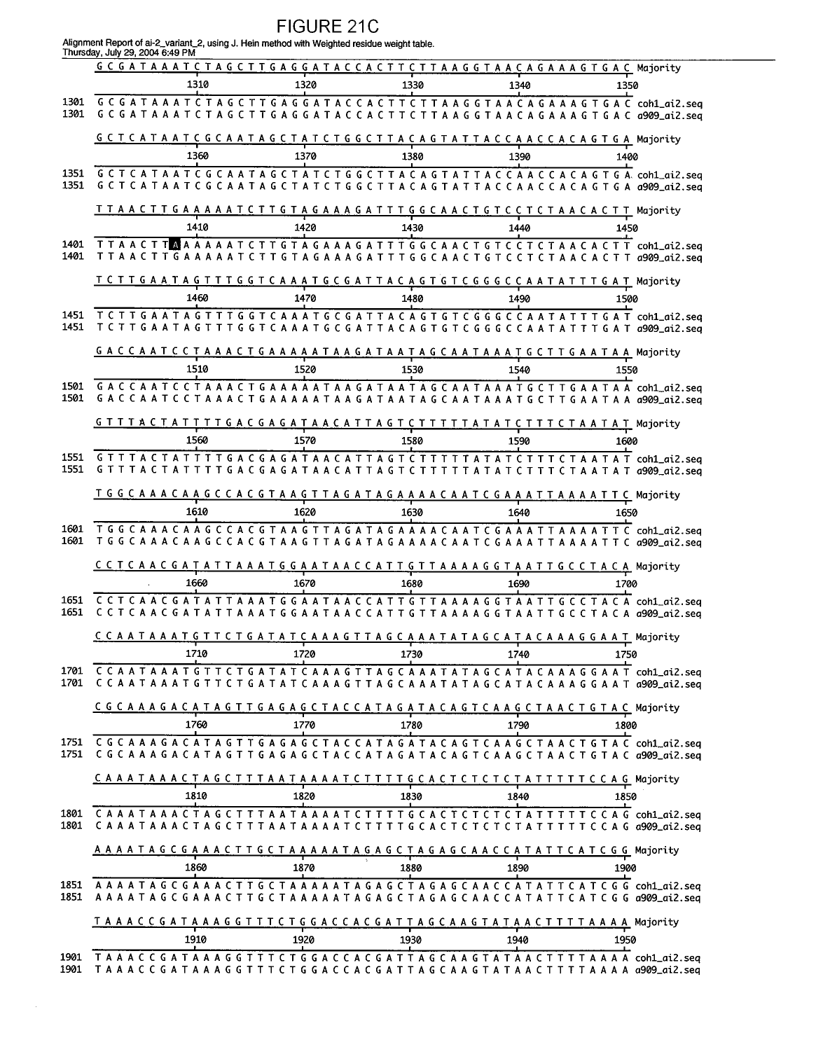

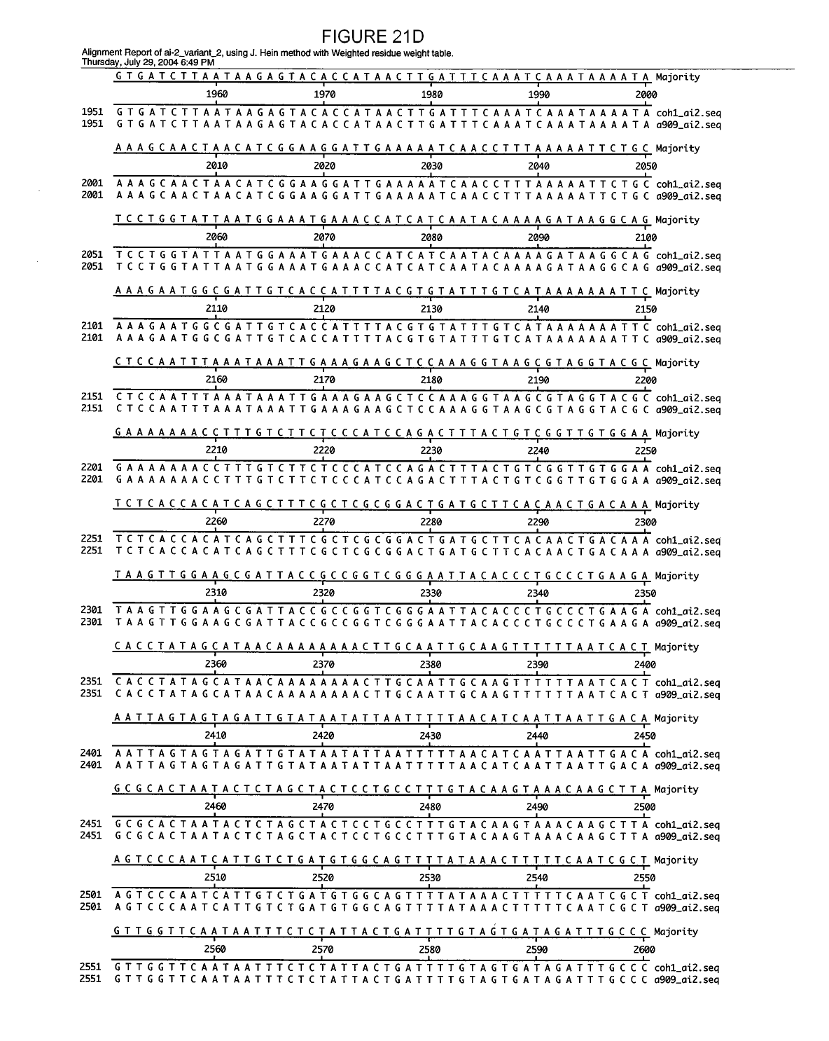

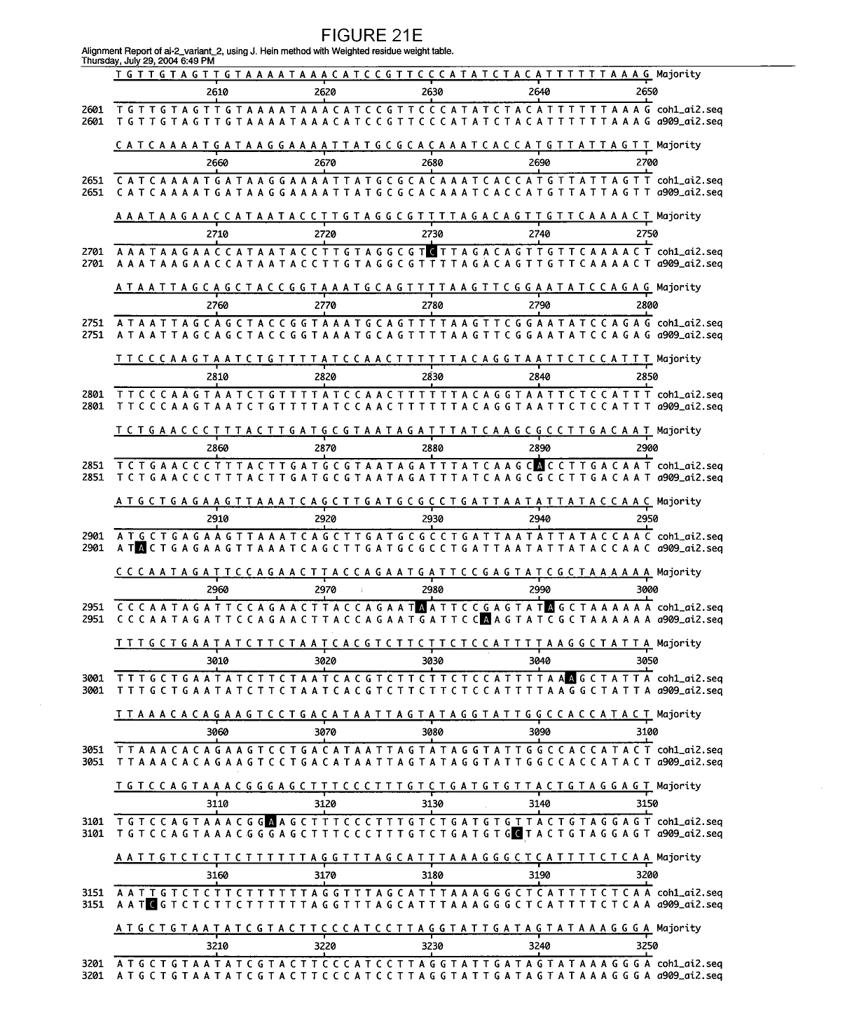

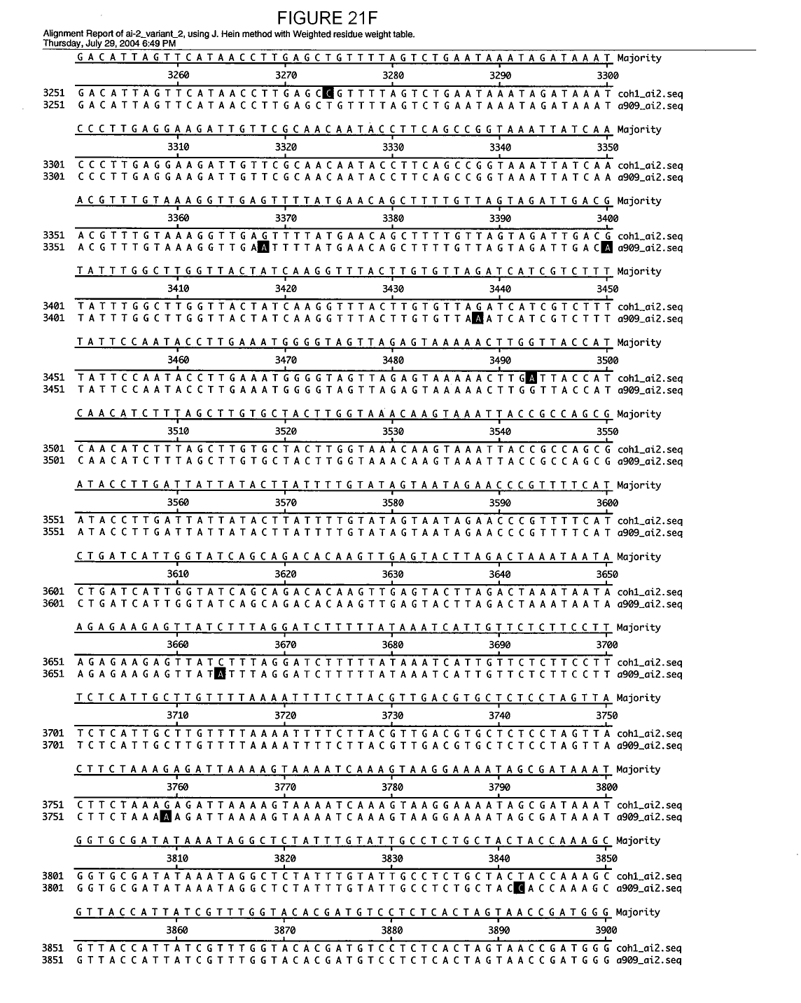

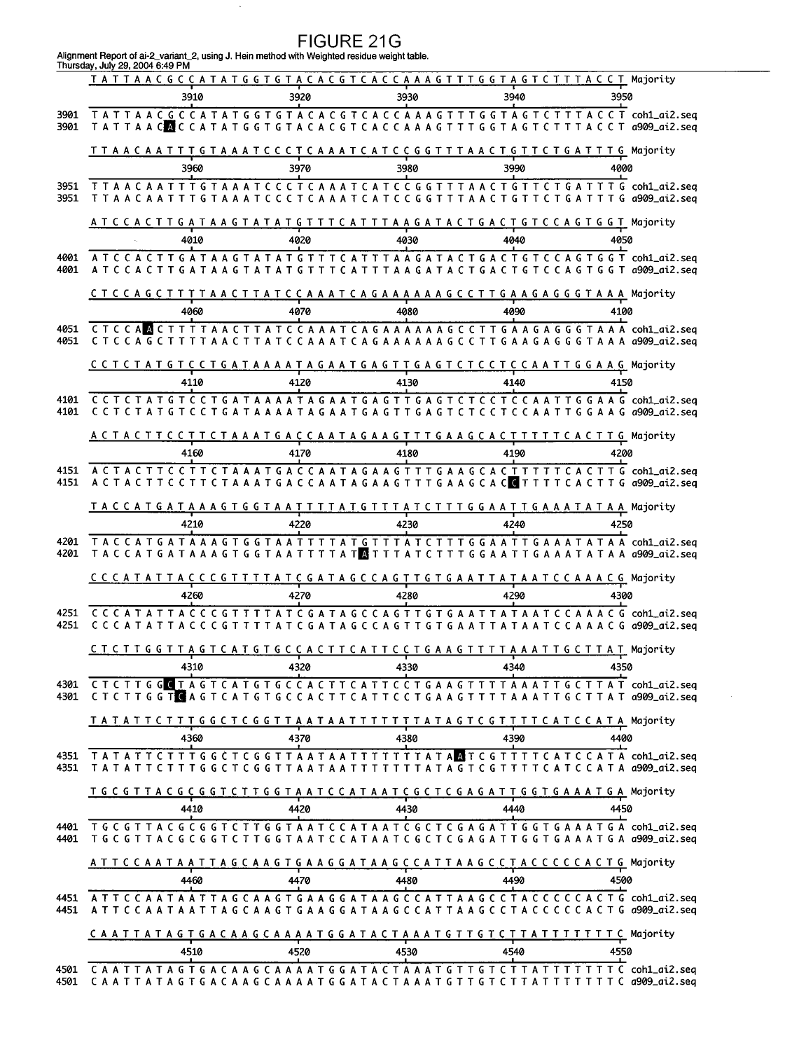

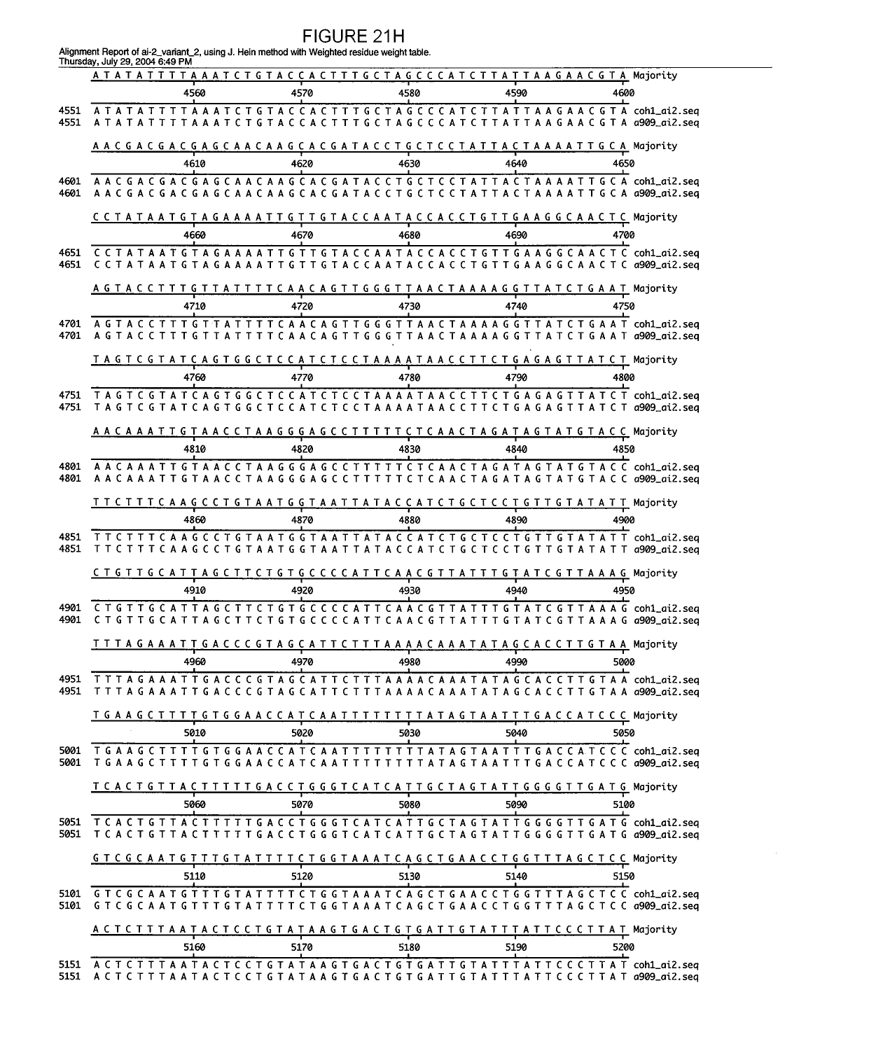

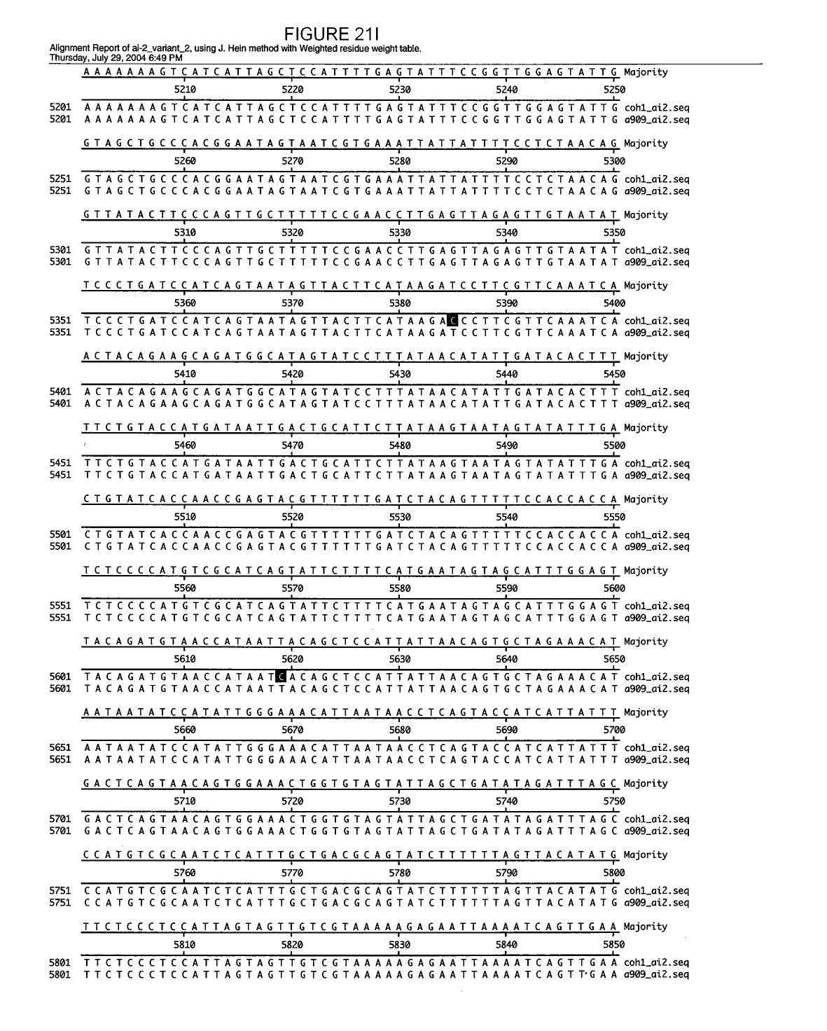

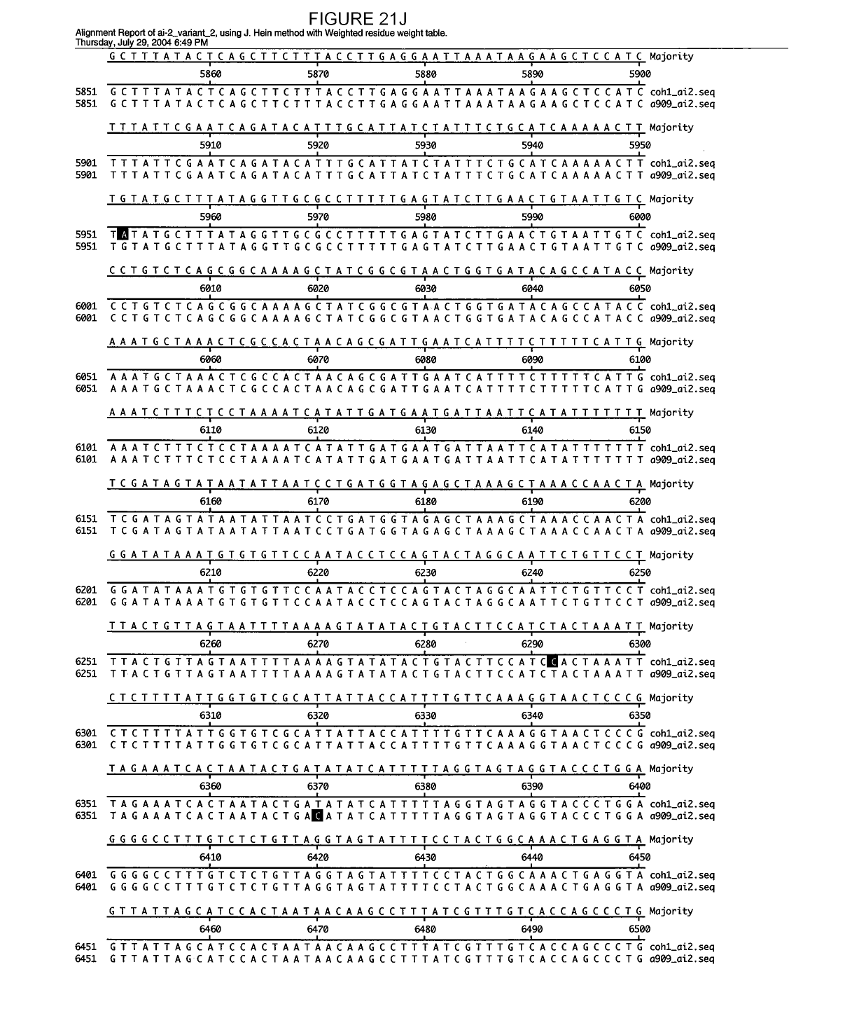

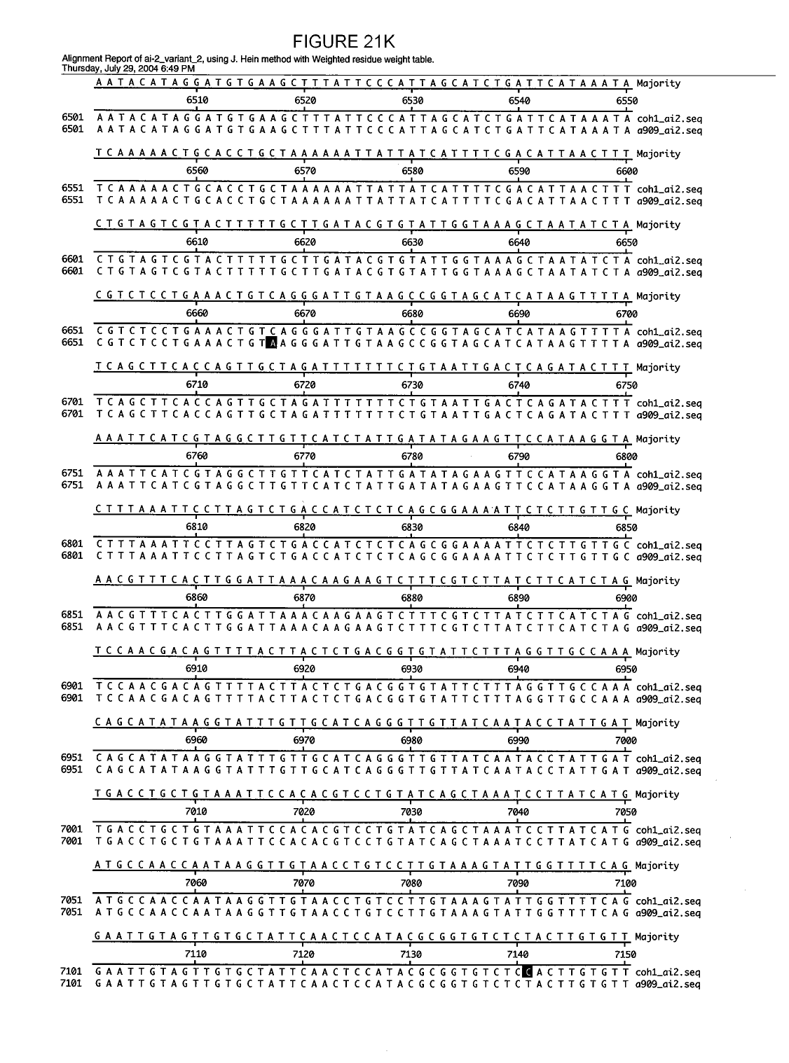

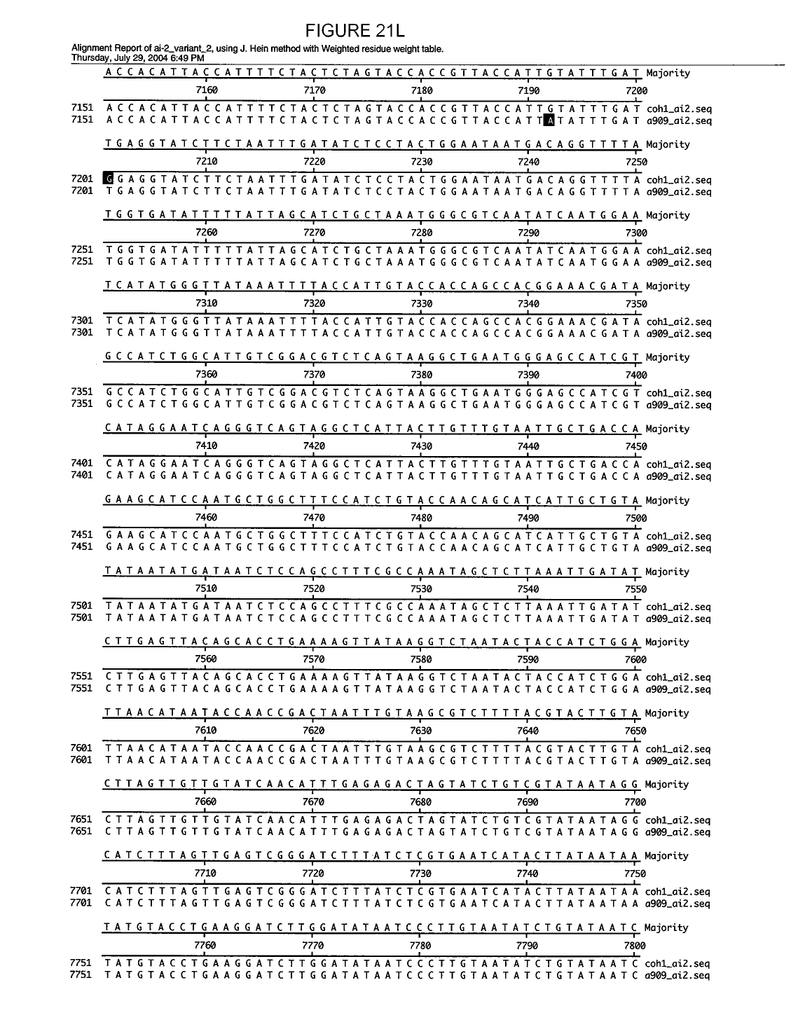

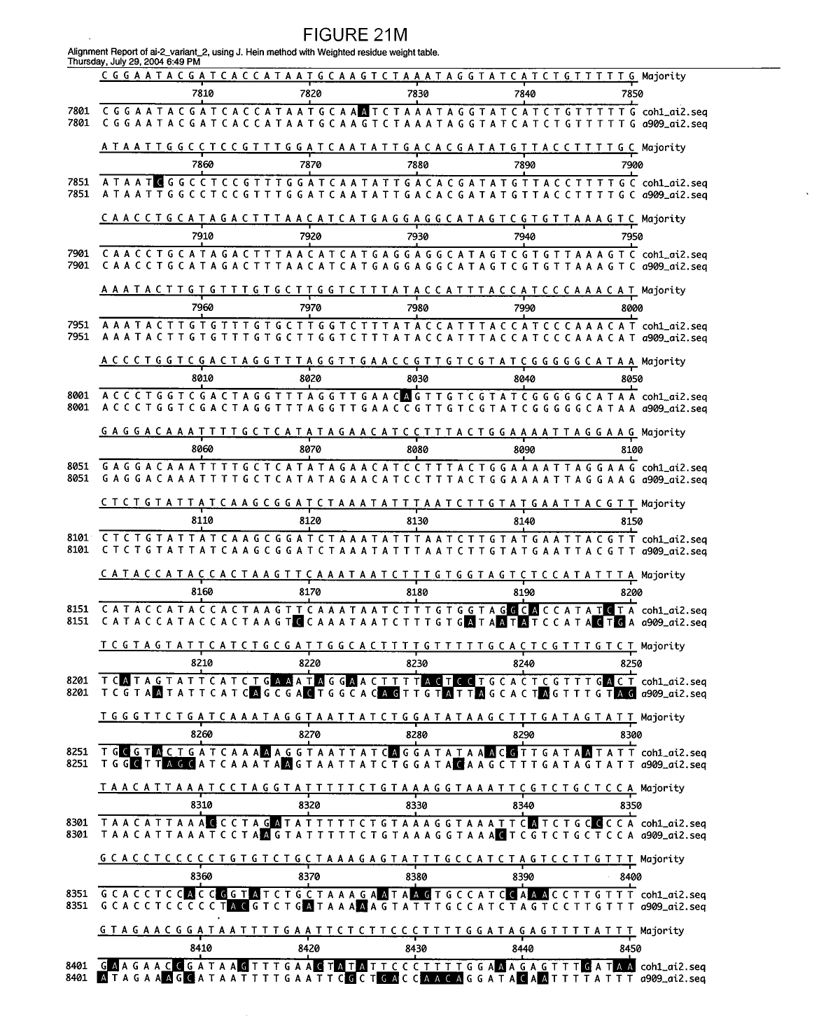

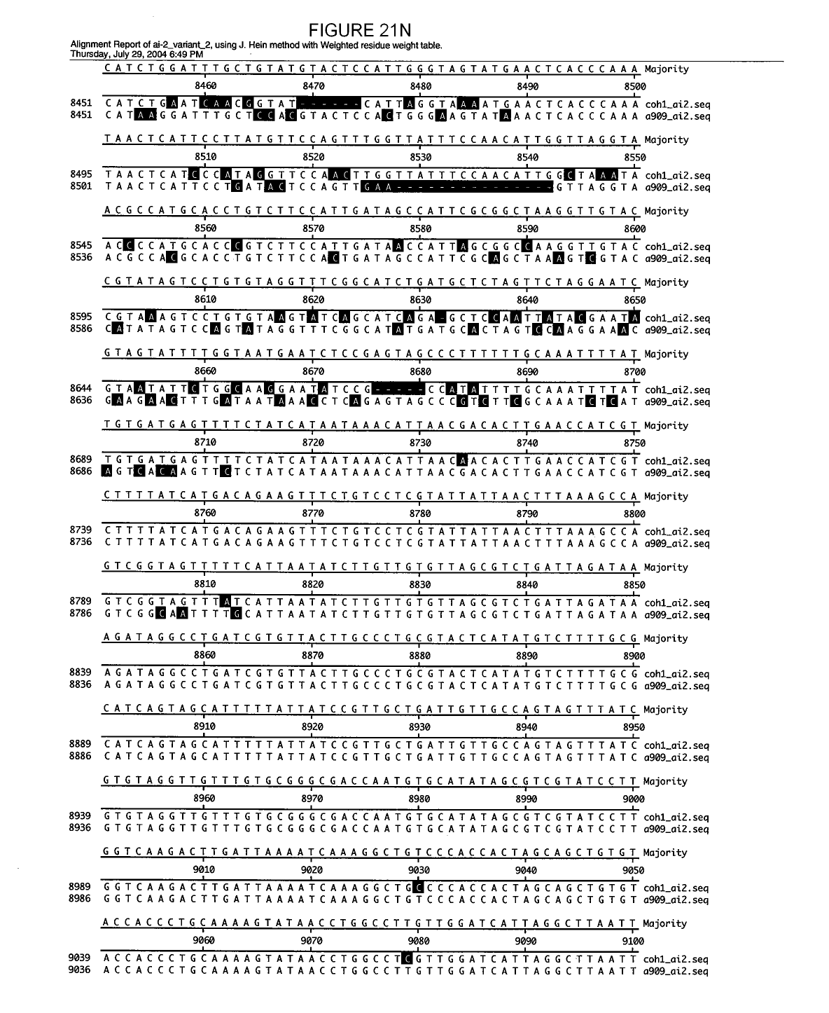

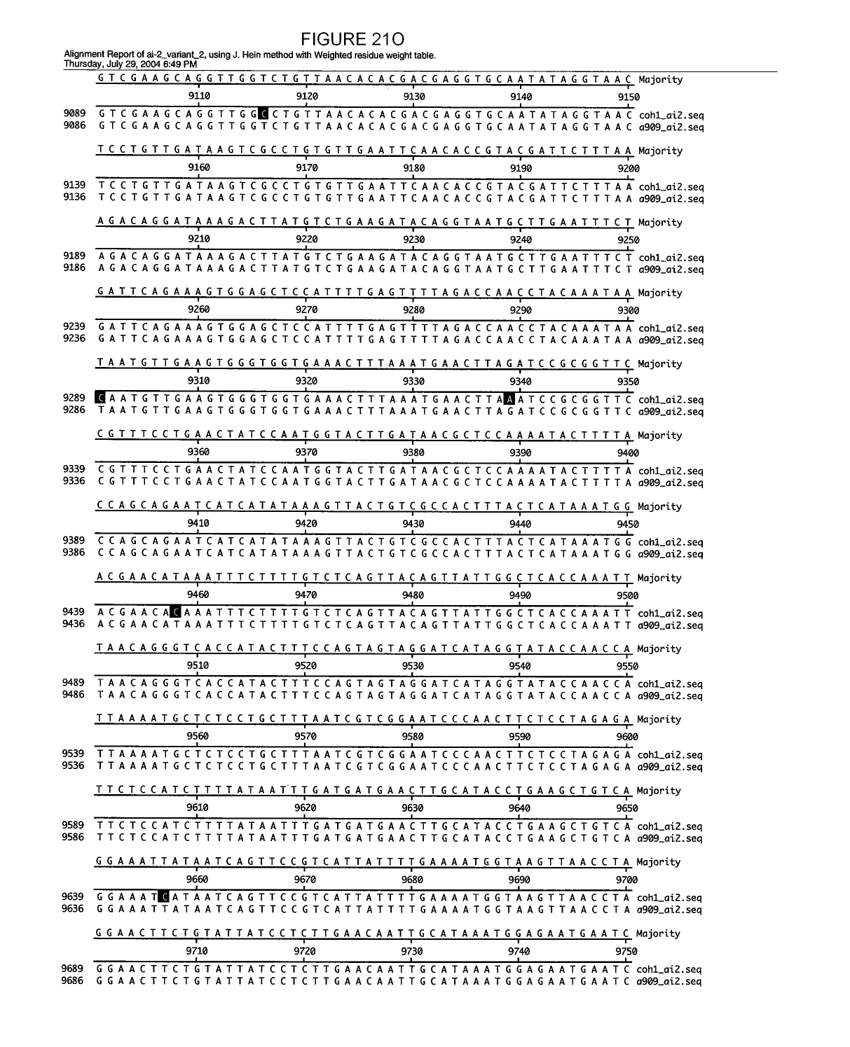

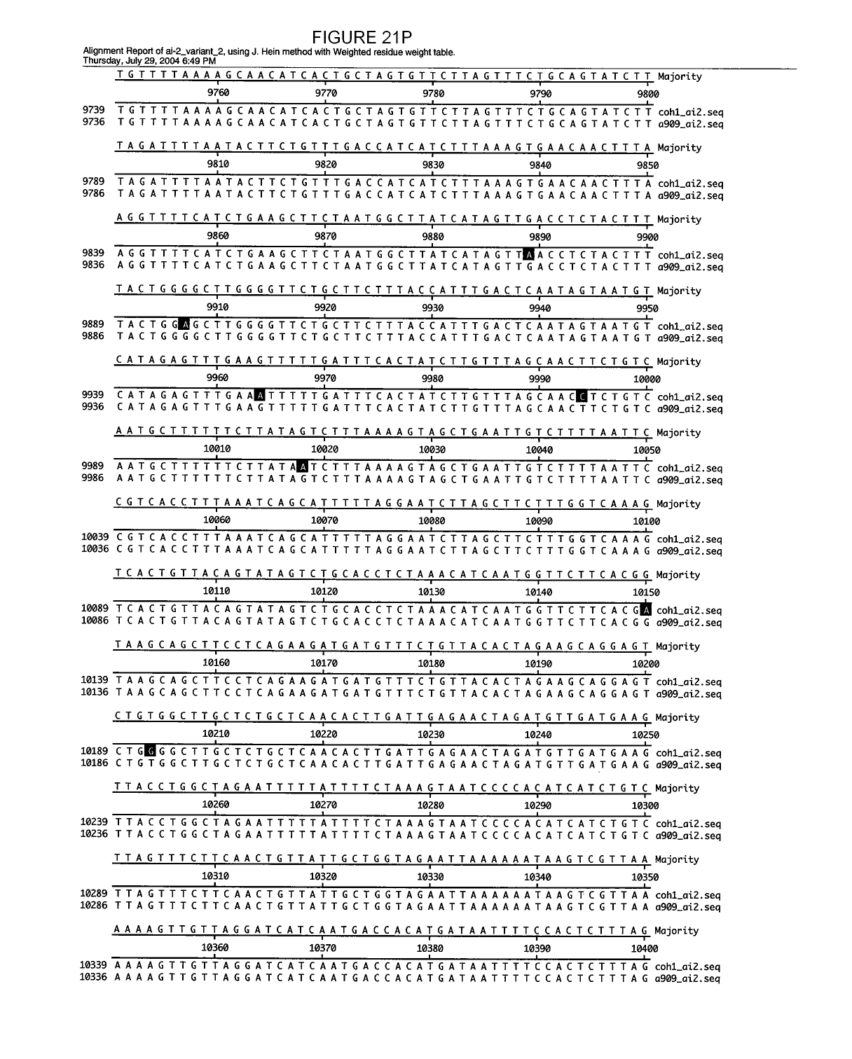

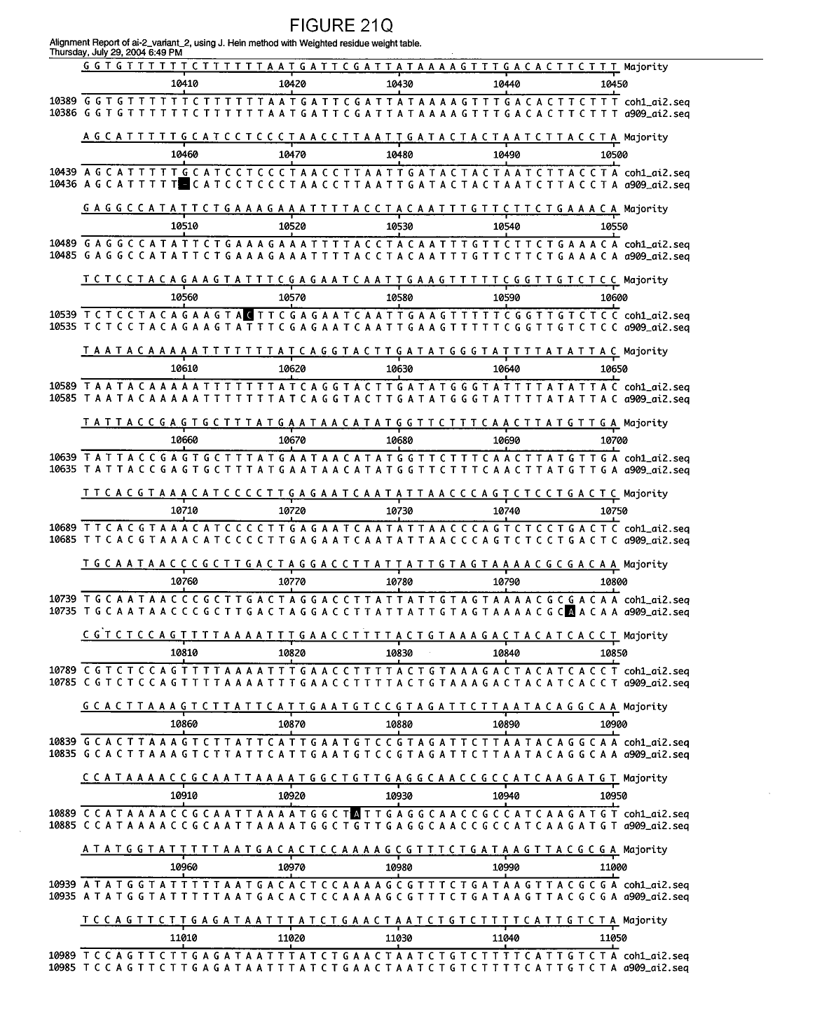

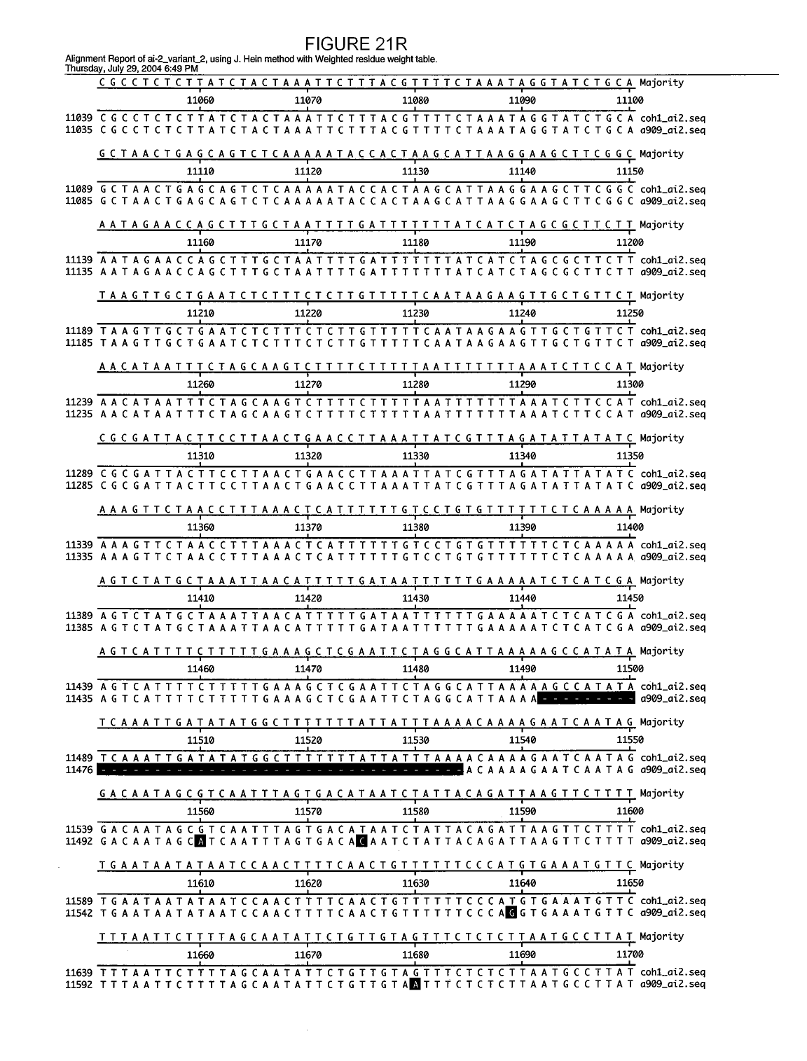

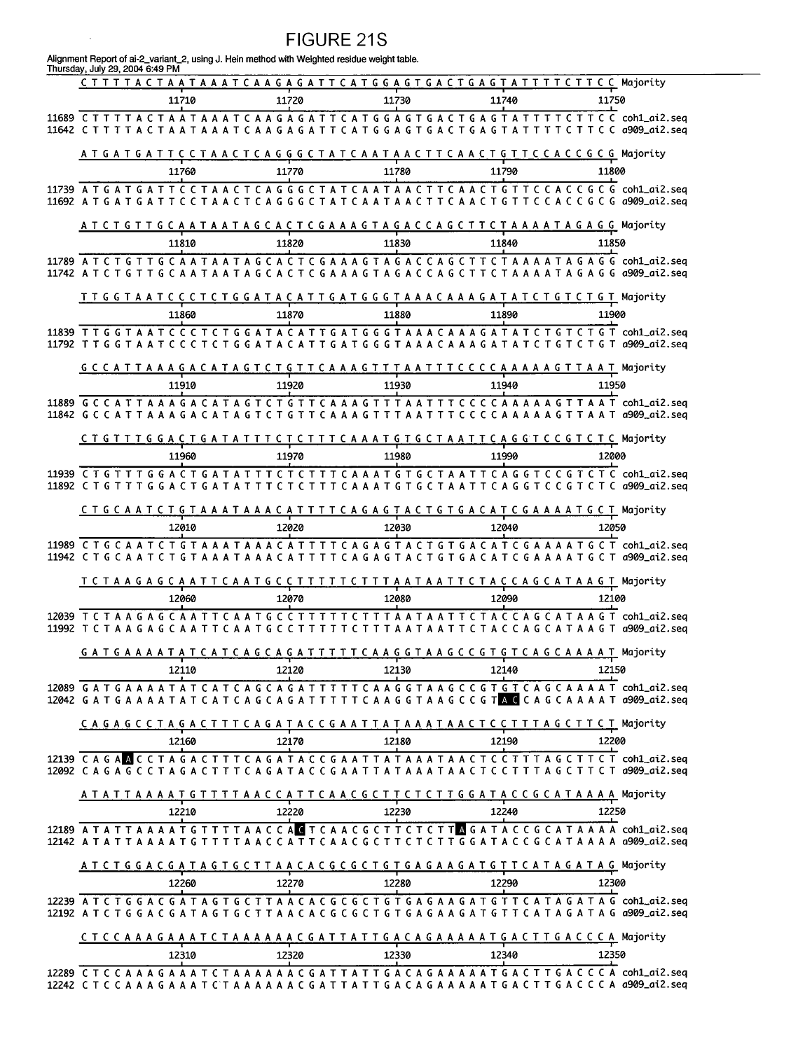

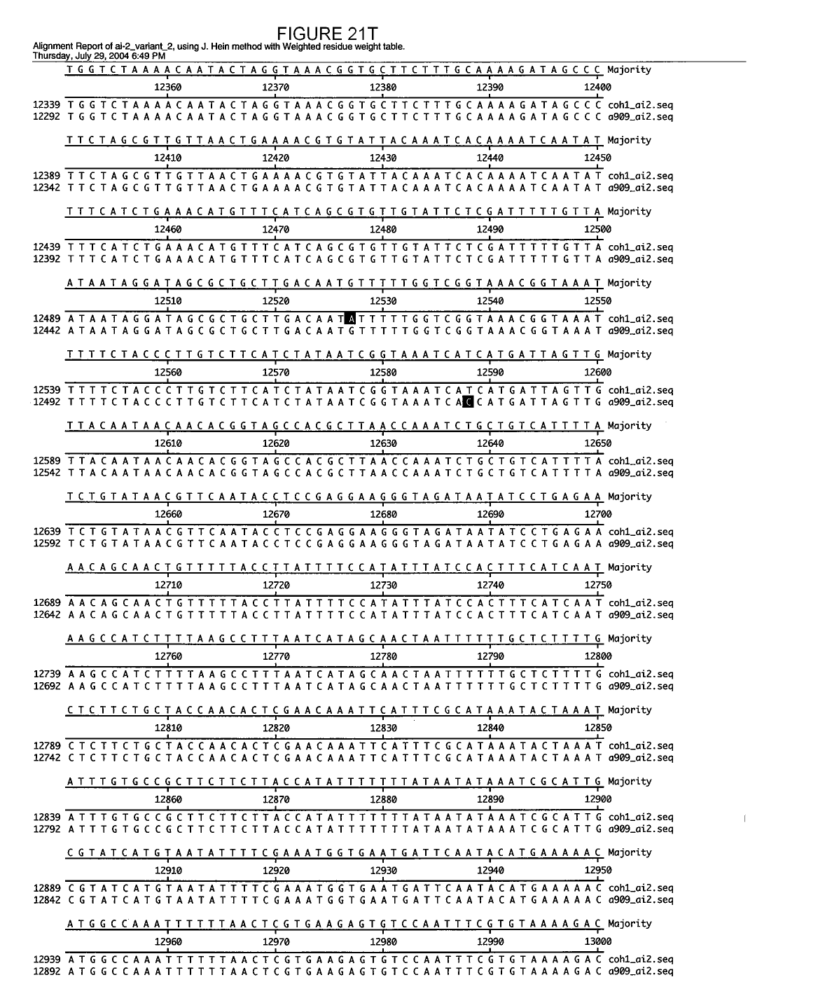

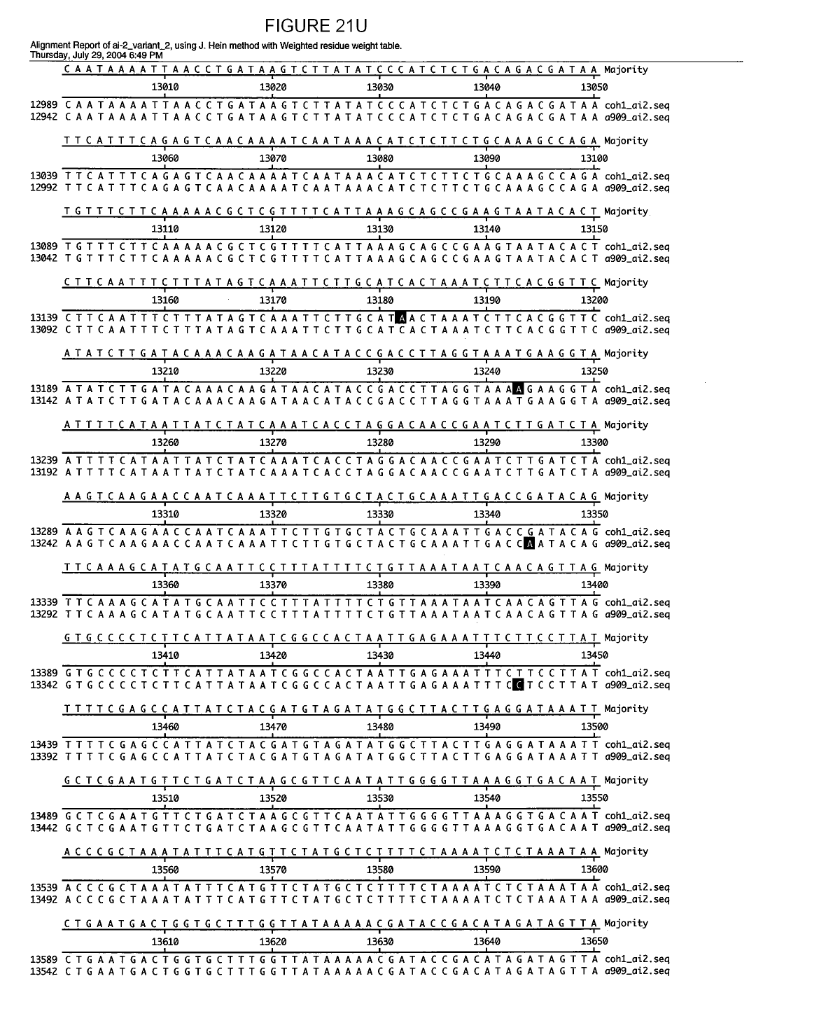

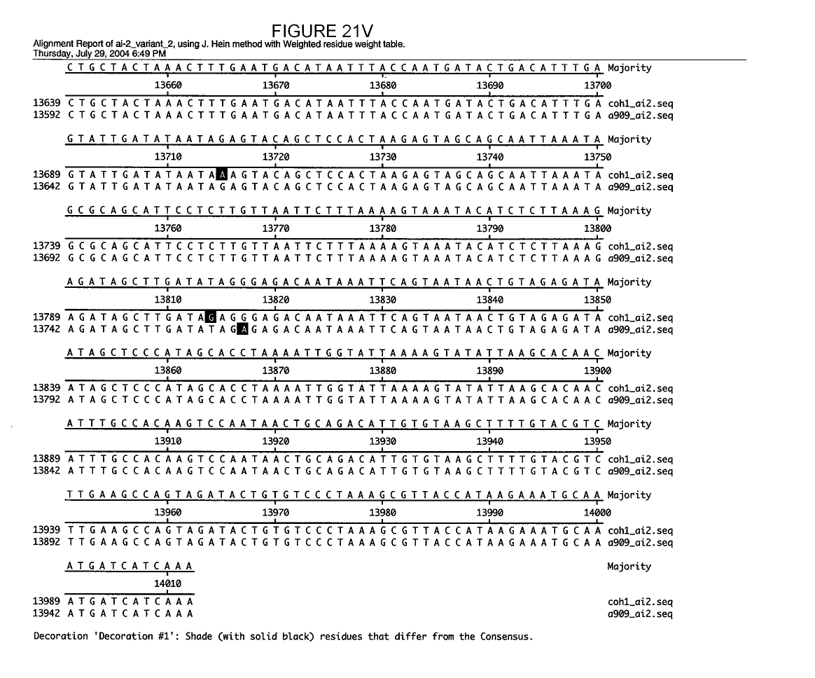

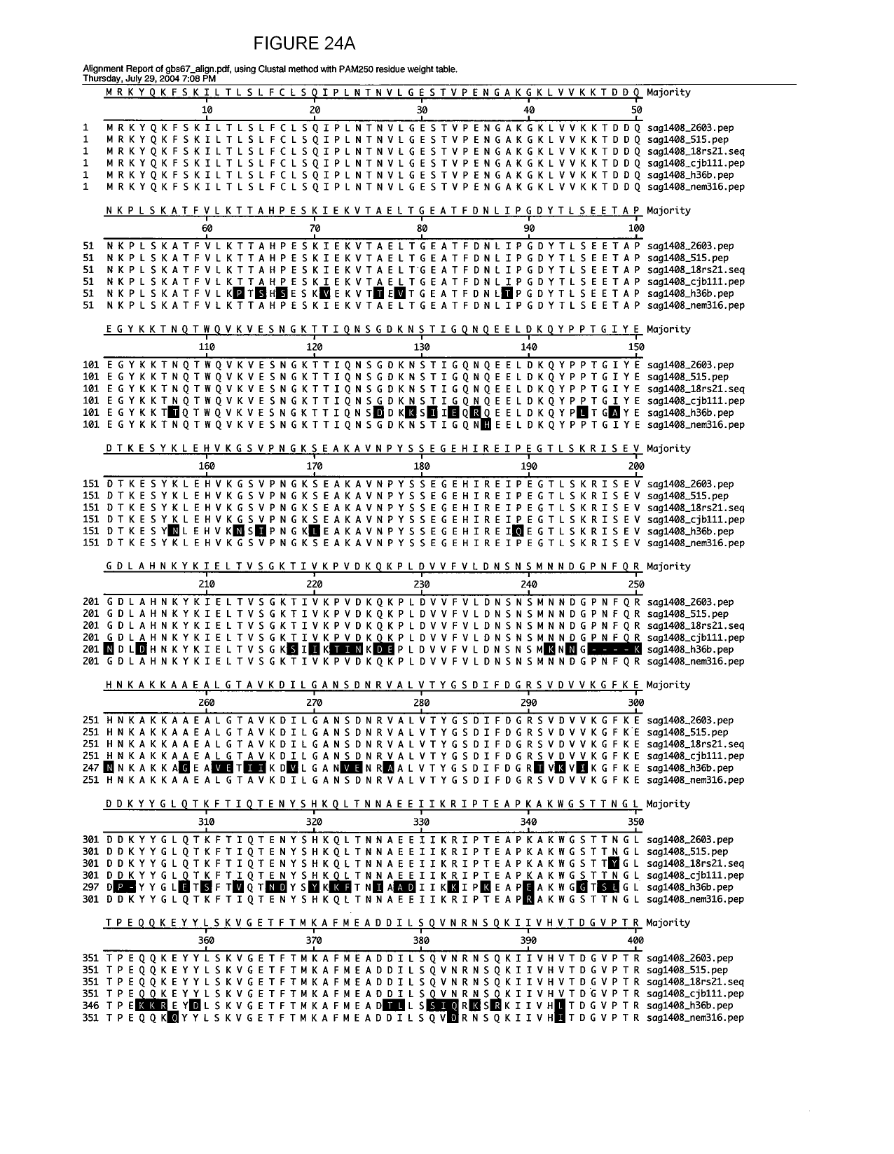

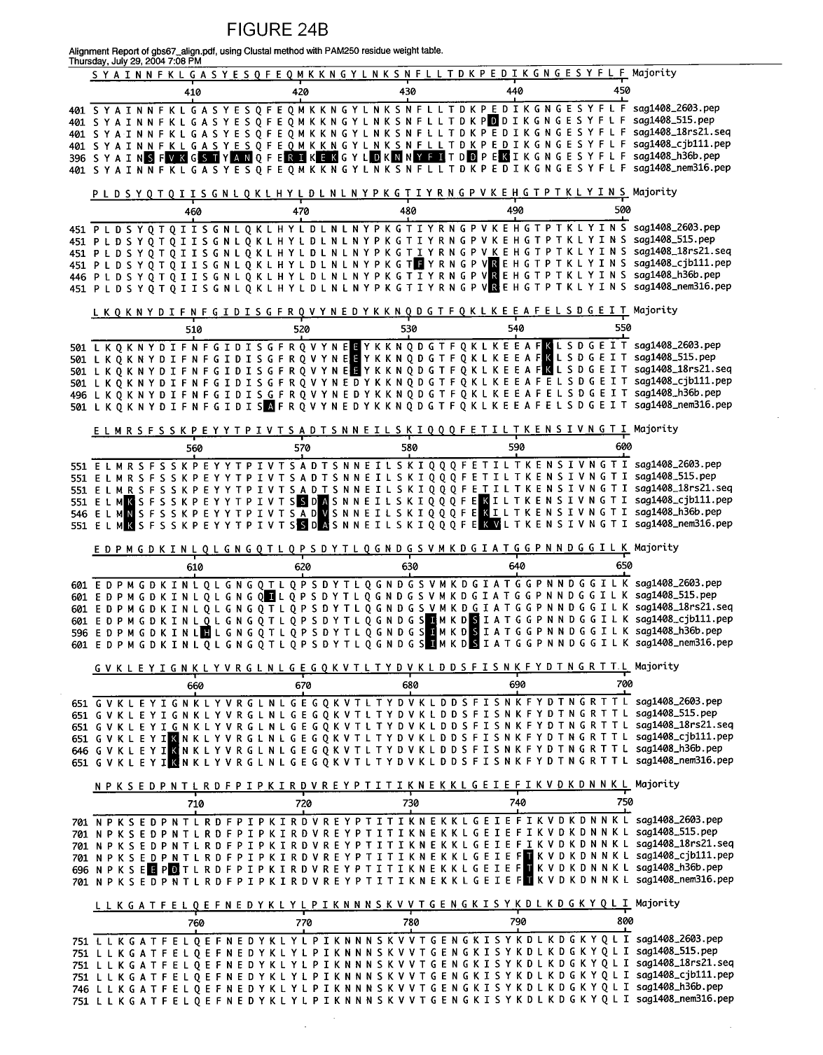

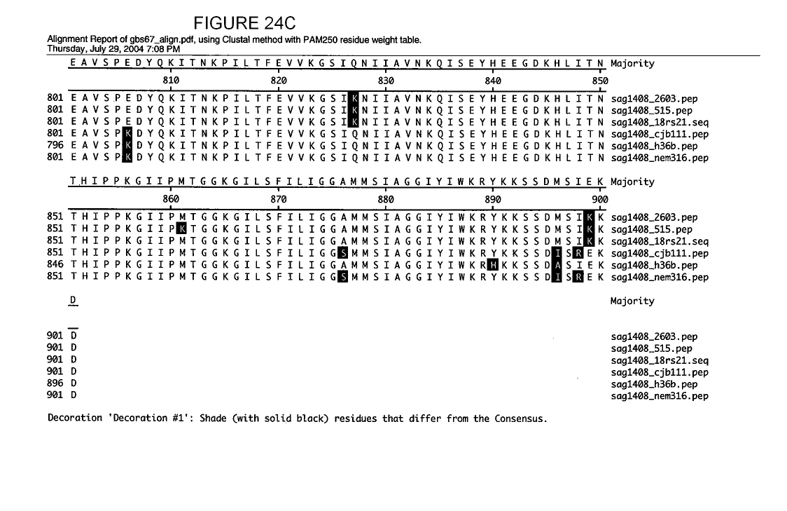

A second adhesin island, “Adhesin Island 2” or “AI-2” or “GBS AI-2” has also been identified in numerous GBS serotypes. A schematic depicting the correlation between AI-1 and AI-2 within the GBS serotype V, strain isolate 2603 is shown in FIG. 3. (Homology percentages in FIG. 3 represent amino acid identity of the AI-2 proteins to the AI-1 proteins). Alignments of AI-2 polynucleotide sequences are presented in FIGS. 19, 20, and 21. FIG. 19 includes sequences from serotype V, strain isolates 2603 and CJB111; serotype II, strain isolate 18RS21; serotype 1b, strain isolate H36B; and serotype 1a, strain isolate 515. FIG. 20 includes sequences from serotype V, strain isolate 2603 and serotype III, strain isolate NEM316. FIG. 21 includes sequences from serotype III, strain isolate COH1 and serotype Ia, strain isolate A909. An alignment of amino acid sequences of AI-2 surface protein GBS 067 from serotype V, strain isolates 2603 and CJB111; serotype 1a, strain isolate 515; serotype II, strain isolate 18RS21; serotype Ib, strain isolate H36B; and serotype III, strain isolate NEM316 is presented in FIG. 24. Preferred AI-2 polynucleotide and amino acid sequences are conserved among two or more GBS serotypes or strain isolates.

AI-2 comprises a series of approximately five open reading frames encoding for a collection of amino acid sequences comprising surface proteins and sortases. Specifically, AI-2 includes open reading frames encoding for two or more (i.e., 2, 3, 4, 5 or more) of GBS 67, GBS 59, GBS 150, SAG1405, SAG1406, 01520, 01521, 01522, 01523, 01523, 01524 and 01525. In one embodiment, AI-2 includes open reading frames encoding for two or more of GBS 67, GBS 59, GBS 150, SAG1405, and SAG1406. Alternatively, AI-2 may include open reading frames encoding for two or more of 01520, 01521, 01522, 01523, 01523, 01524 and 01525.

One or more of the surface proteins typically include an LPXTG motif (e.g., SEQ ID NO:122) or other sortase substrate motif. The GBS AI-2 sortase proteins are thought to be involved in the secretion and anchoring of the LPXTG containing surface proteins. GBS AI-2 may encode for at least one surface protein. Alternatively, AI-2 may encode for at least two surface proteins and at least one sortase. Preferably, GBS AI-2 encodes for at least three surface proteins and at least two sortases. One or more of the AI-2 surface proteins may include an LPXTG or other sortase substrate motif.

One or more of the surface proteins may also typically include pilin motif. The pilin motif may be involved in pili formation. Cleavage of AI surface proteins by sortase between the threonine and glycine residue of an LPXTG motif yields a thioester-linked acyl intermediate of sortase. The first lysine residue in a pilin motif can serve as an amino group acceptor of the cleaved LPXTG motif and thereby provide a covalent linkage between AI subunits to form pili. For example, the pilin motif can make a nucleophilic attack on the acyl enzyme providing a covalent linkage between AI subunits to form pili and regenerate the sortase enzyme. Some examples of pilin motifs that may be present in the GBS AI-2 proteins include ((YPKN(X₈)K; SEQ ID NO:158), (PK(X₈)K; SEQ ID NO:159), (YPK(X₉)K;SEQ ID NO:160), (PKN(X₈)K; SEQ ID NO:161), or (PK(X₁₀)K; SEQ ID NO:162)).

One or more of the surface protein may also include an E box motif. The E box motif contains a conserved glutamic acid residue that is believed to be necessary for pilus formation. Some examples of E box motifs may include the amino acid sequences YxLxETxAPxG (SEQ ID NO:163), YxxxExxAxxGY (SEQ ID NO:164), YxLxExxxPxDY (SEQ ID NO:165), or YxLxETxAPxGY (SEQ ID NO:152).

As shown in FIG. 3, GBS AI-2 may include the surface exposed proteins of GBS 67, GBS 59 and GBS 150 and the sortases of SAG1406 and SAG1405. Alternatively, GBS AI-2 may include the proteins 01521, 01524 and 01525 and sortases 01520 and 01522. GBS 067 and 01524 are preferred AI-2 surface proteins.

AI-2 may also include a divergently transcribed transcriptional regulator such as a RofA like protein (for example rogB). As in AI-1, rogB is thought to regulate the expression of the AI-2 operon.

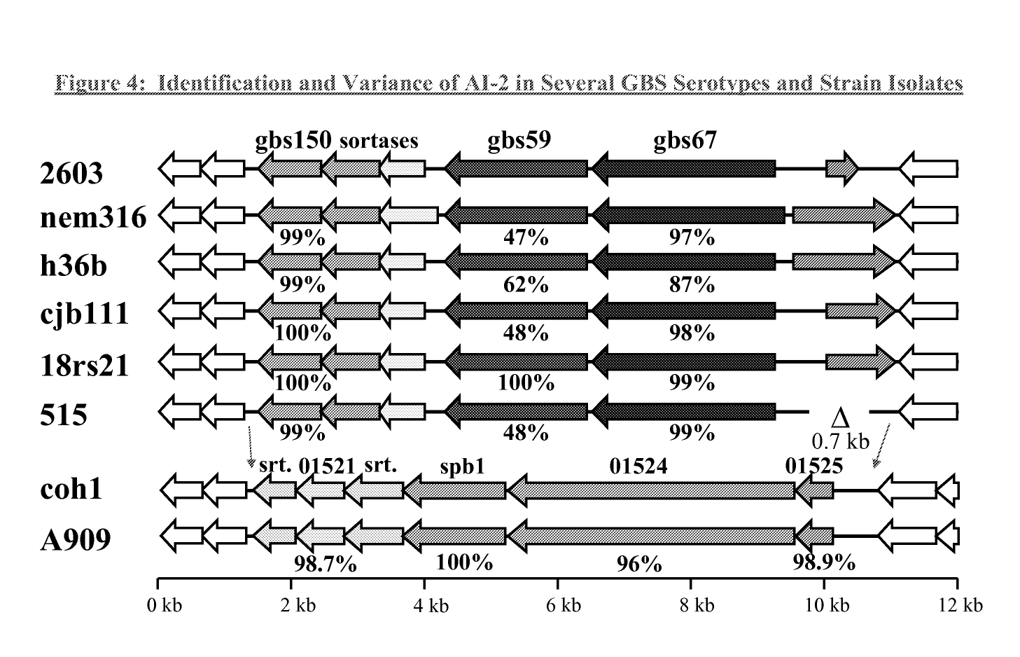

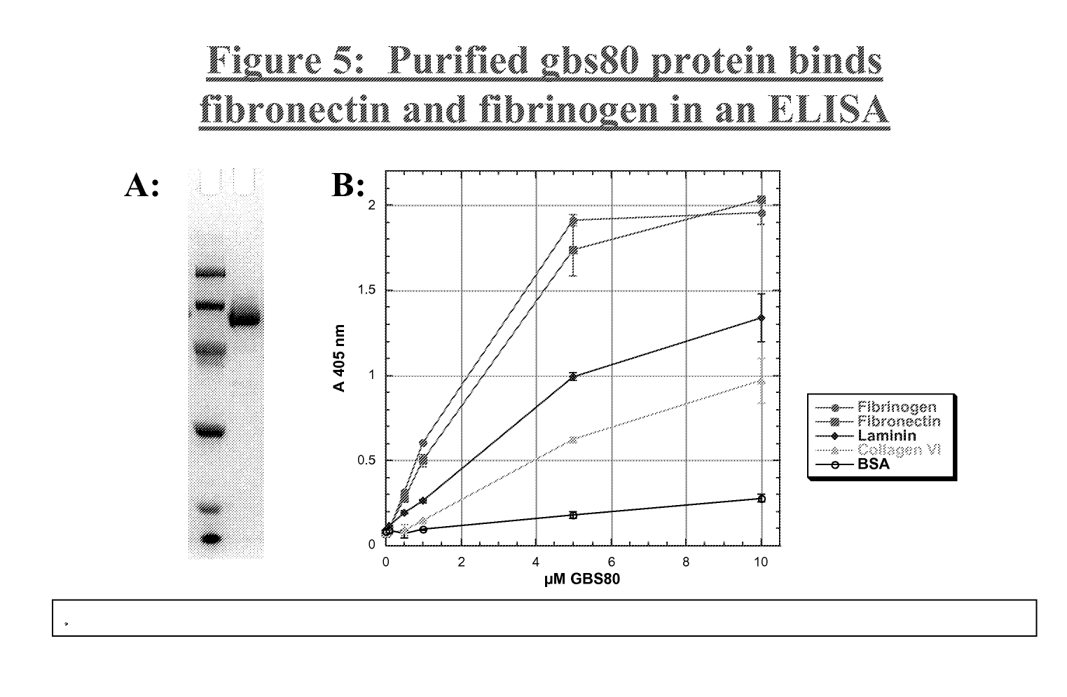

A schematic depiction of AI-2 within several GBS serotypes is depicted in FIG. 4. (Percentages shown are amino acid identity to the 2603 sequence). While the AI-2 surface proteins GBS 59 and GBS 67 are more variable across GBS serotypes than the corresponding AI-1 surface proteins, AI-2 surface protein GBS 67 appears to be conserved in GBS serotypes where the AI-1 surface proteins are disrupted or missing.

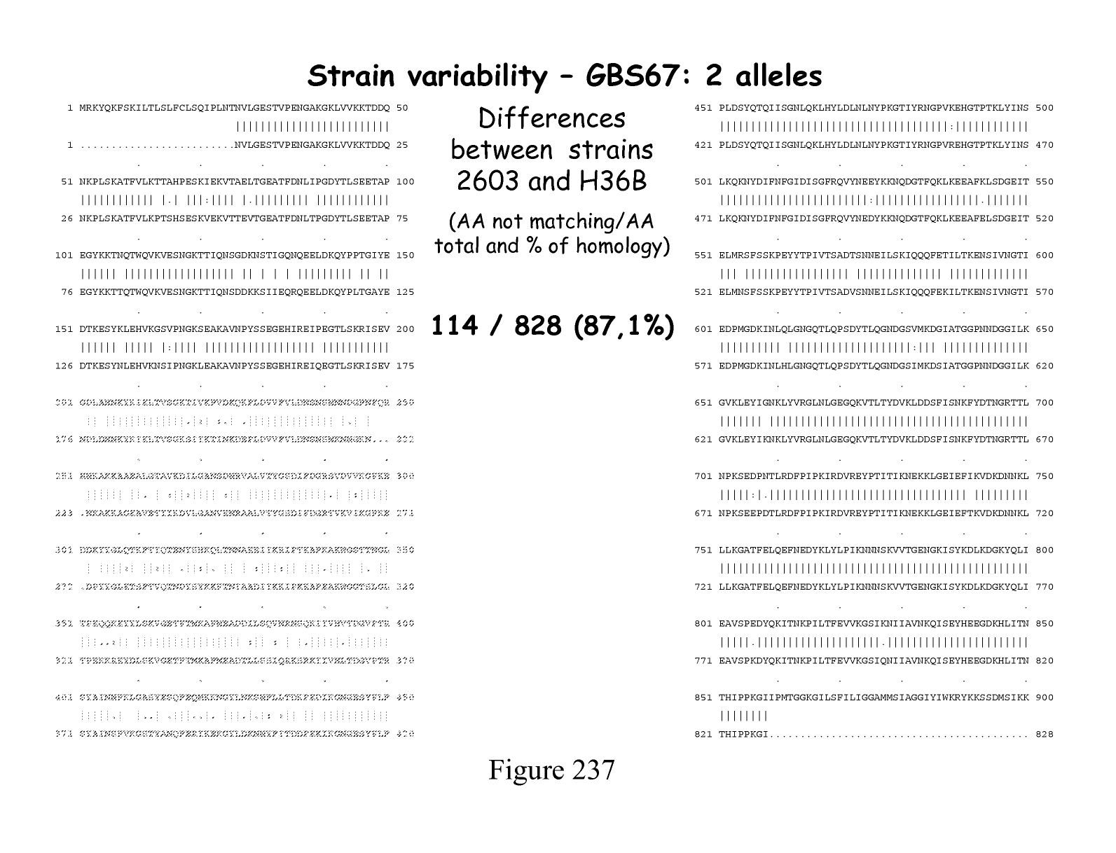

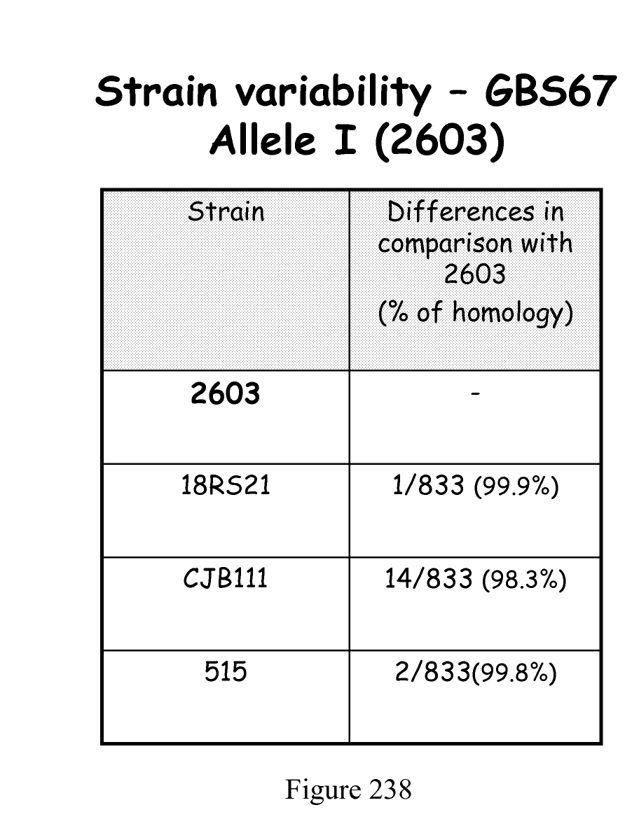

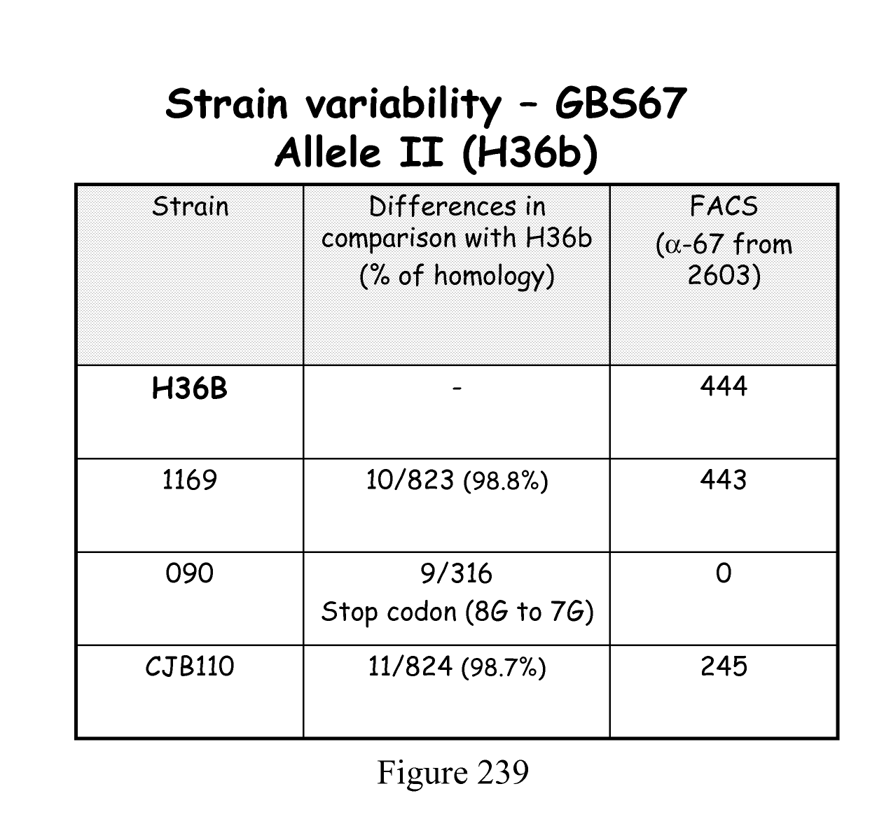

For example, as discussed above and in FIG. 2, the AI-1 GBS 80 surface protein is fragmented in GBS serotype II, strain isolate 18RS21. Within AI-2 for this same sequence, as shown in FIG. 4, the GBS 67 surface protein has 99% amino acid sequence homology with the corresponding sequence in strain isolate 2603. Similarly, the AI-1 GBS 80 surface protein appears to be missing in GBS serotype Ib, strain isolate H36B and GBS serotype Ia, strain isolate 515. Within AI-2 for these sequences, however, the GBS 67 surface protein has 97-99% amino acid sequence homology with the corresponding sequence in strain isolate 2603. GBS 67 appears to have two allelic variants, which can be divided according to percent homology with strains 2603 (GBS67²⁶⁰³) and H36B (GBS67^{H36B}). See FIGS. 237-239.

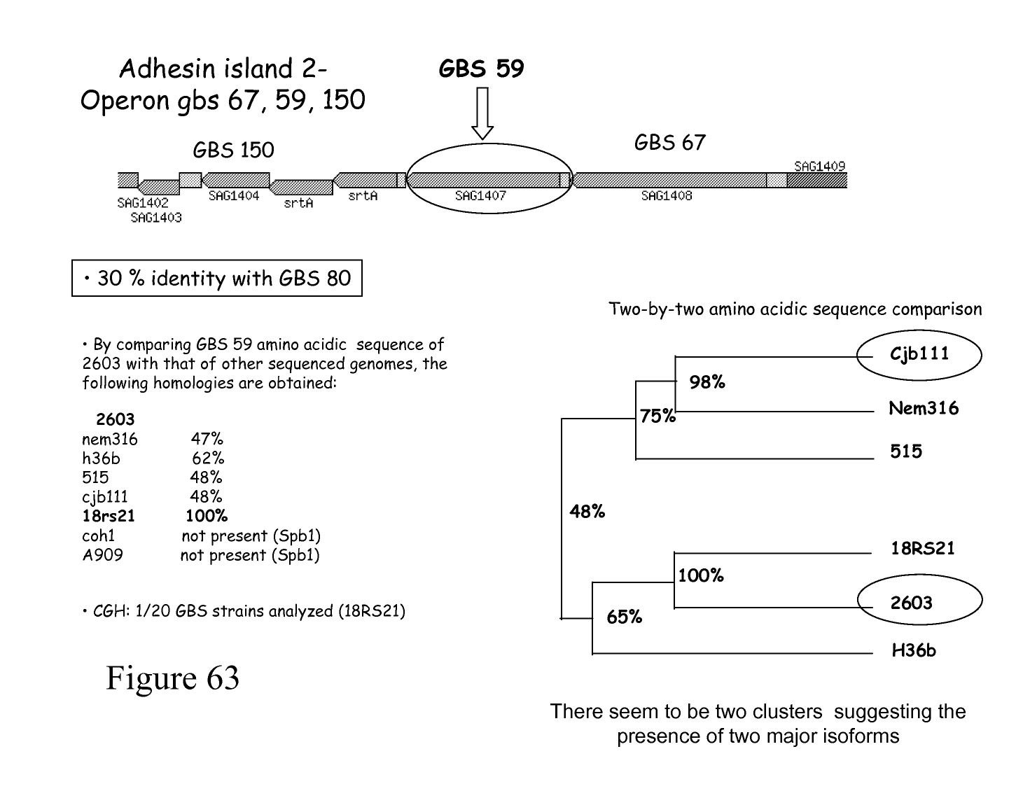

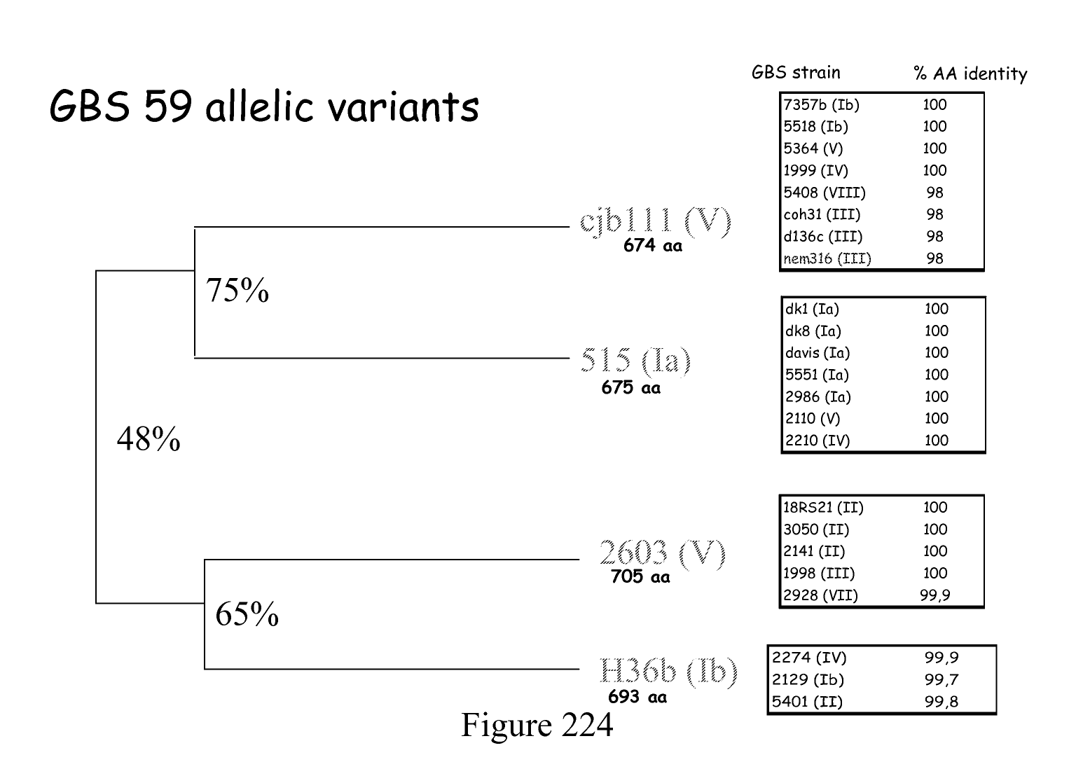

Unlike for GBS 67, amino acid sequence identity of GBS 59 is variable across different GBS strains. As shown in FIGS. 63 and 224, GBS 59 of GBS strain isolate 2603 shares 100% amino acid residue homology with GBS strain 18RS21, 62% amino acid sequence homology with GBS strain H36B, 48% amino acid residue homology with GBS strain 515 and GBS strain CJB111, and 47% amino acid residue homology with GBS strain NEM316. The amino acid sequence homologies of the different GBS strains suggest that there are two isoforms of GBS 59. The first isoform appears to include the GBS 59 protein of GBS strains CJB111, NEM316, and 515 (GBS59^{CJB111}, GBS59^{NEM316} and GBS59⁵¹⁵ respectively). The second isoform appears to include the GBS 59 protein of GBS strains 18RS21, 2603, and H36B. GBS59^{18RS21}, GBS59²⁶⁰³ and GBS59^{H36B} respectively. See FIGS. 63 and 224. All newly sequenced strains were deposited at American Type Culture Collection under the following accession numbers: A909, BAA-1138; CJB111, BAA-23; H36b, BAA-1174; 18RS21, BAA-1175; COH1, BAA-1176; and 515, BAA-1177. References for the eight strains are as follows: NEM316 (Glaser et al., Mol. Microbiol. 45, 1499-1513, 2002); 2603V/R (Tettelin et al., Proc. Nati. Acad. Sci. USA 99, 12391-96, 2002); A909, H36B, and 18RS21 (Lancefield et al., J. Exp. Med. 142, 165-79, 1975); 515 (Wessels et al., Infect. Immun. 61, 4760-66, 1993); COH1 (Wilson & Weaver, J. Infect. Dis. 152, 323-29, 1985); and CJB111 (Carol Baker Collection, Division of Infectious Diseases, Baylor College of Medicine, Houston). From Tettelin et al., Proc. Natl. Acad. Sci. USA 102, 13950-55, 2005.

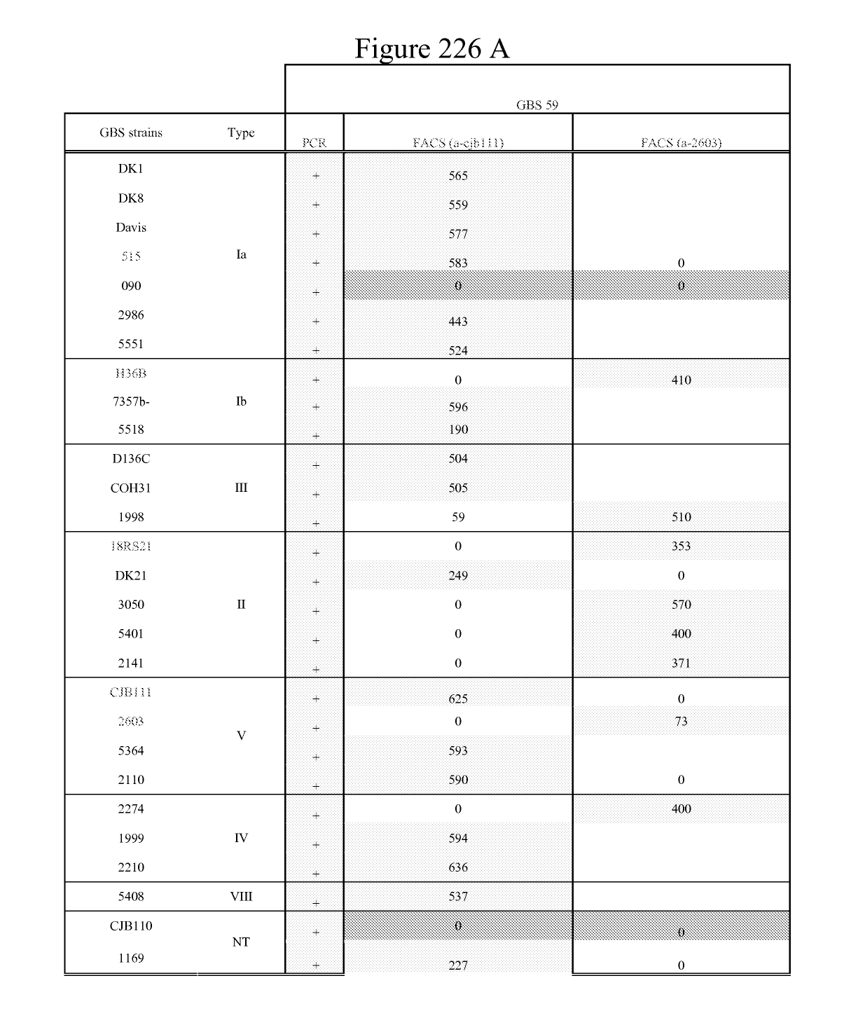

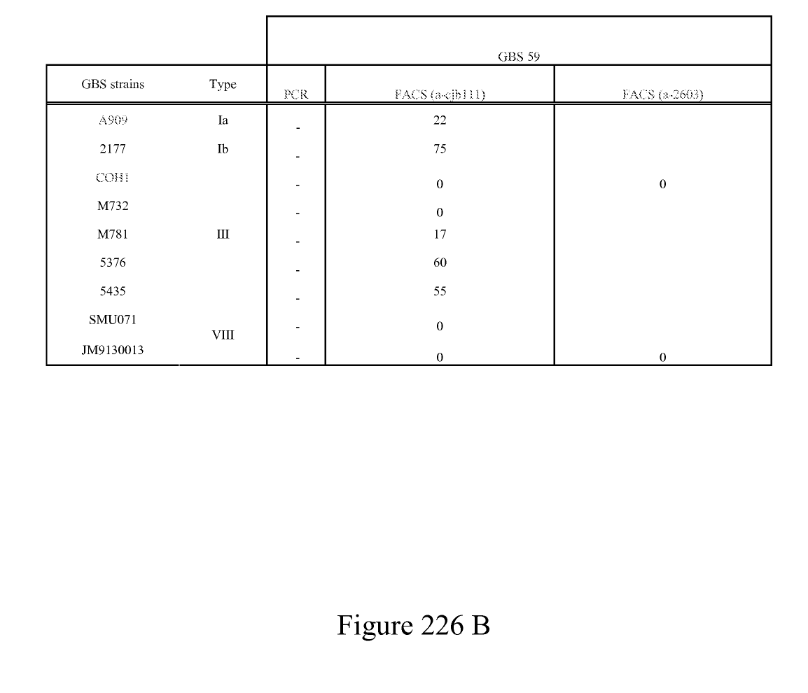

As expected from the variability in GBS 59 isoforms, antibodies specific for the first GBS 59 isoform detect the first but not the second GBS 59 isoform and antibodies specific for the second GBS 59 isoform detect the second but not the first GBS 59 isoform. See FIG. 226A, which shows FACS analysis of 28 GBS strains having a GBS 59 gene detected using PCR for GBS 59 surface expression. For each of the 28 GBS strains, FACS analysis was performed using either an antibody for GBS 59 isoform 1 (α-cjb111) or GBS 59 isoform 2 (α-2603). Only one of the two antibodies detected GBS 59 surface expression on each GBS strain. As a negative control, GBS strains in which a GBS 59 gene was not detectable by PCR did not have significant GBS 59 surface expression levels. FIG. 226B.

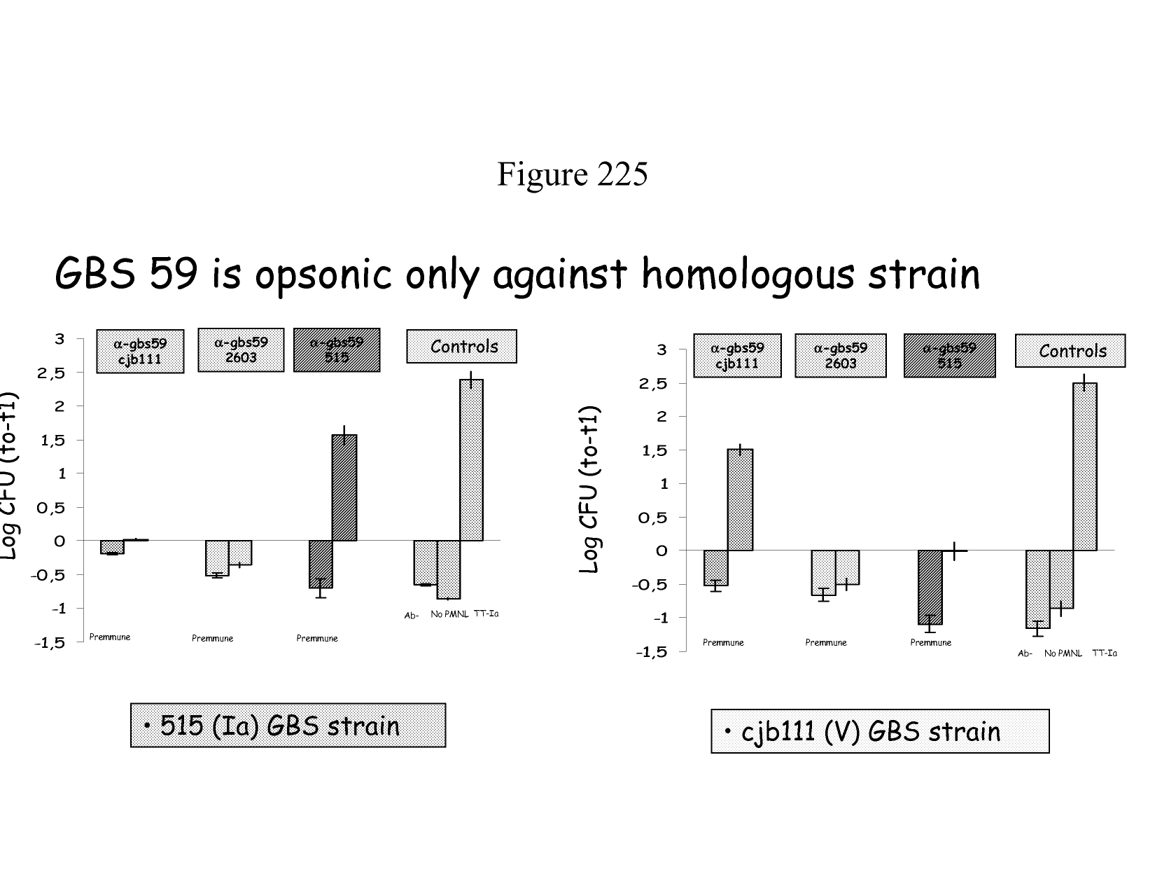

Also, GBS 59 is opsonic only against GBS strains expressing a homologous GBS 59 protein. See FIG. 225.

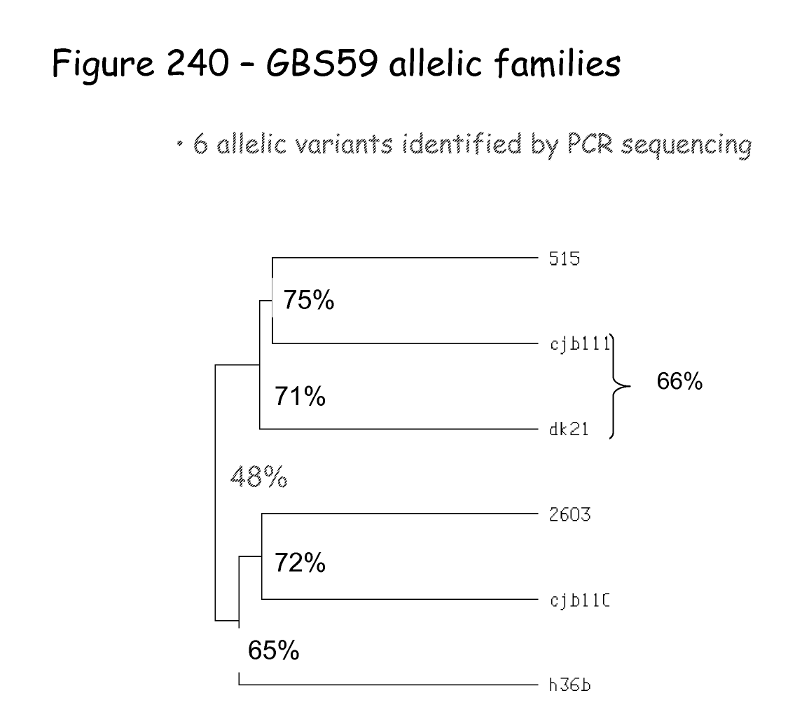

In one embodiment, the immunogenic composition of the invention comprises a first and a second isoform of the GBS 59 protein to provide protection across a wide range of GBS serotypes that express polypeptides from a GBS AI-2. The first isoform may be the GBS 59 protein of GBS strain CJB111, NEM316, or 515 (i.e., GBS59^{CJB111}, GBS59^{NEM316} and GBS59⁵¹⁵). The second isoform may be the GBS 59 protein of GBS strain 18RS21, 2603, or H36B (i.e., GBS59^{18RS21}, GBS59²⁶⁰³ and GBS59^{H36B})To further investigate GBS59 distribution, presence of GBS59 gene in 80 different GBS isolates was assessed by PCR and the resulting amplicons were sequenced. Table 53 summarizes the sequence analysis results for the 65 positive strains (81%). The various GBS59 sequences thus obtained suggest that GBS59 isoforms can be further grouped in 6 main allelic families, as schematized in FIG. 240. Each sequence member of an allelic family has been compared to the first representative strain in the list of different isolates (i.e., GBS59^{CJB111}, GBS59^{DK21}, GBS59⁵¹⁵, GBS59^{CJB110}, GBS59²⁶⁰³ and GBS59^{II36B}).

TABLE 53

| | | GBS 59 | | |
|---|---|---|---|---|
| GBS strains | Type | variant | % identity | aa not matching/aa total |
| 515 | Ia | 515 | 100 | |
| DK1 | | 515 | 100 | 0/675 |
| DK8 | | 515 | 100 | 0/675 |
| Davis | | 515 | 100 | 0/675 |
| 2986 | | 515 | 100 | 0/675 |
| 5551 | | 515 | 100 | 0/675 |
| cdc-11 | | 515 | 100 | 0/675 |
| cdc-13 | | 515 | 100 | 0/675 |
| cdc-22 | | 515 | 100 | 0/675 |
| cdc-31 | | 515 | 100 | 0/675 |
| cdc-25 | | 515 | 100% + 11aa | 0/675 + 11aa |
| 2110 | V | 515 | 100 | 0/675 |
| 2210 | IV | 515 | 100 | 0/675 |
| CJB111 | V | CJB111 | 100 | |
| 5364 | | CJB111 | 100 | 0/674 |
| cdc-4 | | CJB111 | 100 | 0/674 |
| cdc-6 | | CJB111 | 100 | 0/674 |
| cdc-12 | | CJB111 | 100 | 0/674 |
| cdc-16 | | CJB111 | 100 | 0/674 |
| cdc-20 | | CJB111 | 100 | 0/674 |
| cdc-23 | | CJB111 | 100 | 0/674 |
| cdc-28 | | CJB111 | 100 | 0/674 |
| cdc-30 | | CJB111 | 100 | 0/674 |
| cdc-35 | | CJB111 | 100 | 0/674 |
| cdc-40 | | CJB111 | 100 | 0/674 |
| 7357b- | Ib | CJB111 | 100 | 0/674 |
| 5518 | | CJB111 | 100 | 0/674 |
| cdc-2 | | CJB111 | 100 | 0/674 |
| cdc-36 | | CJB111 | 100 | 0/674 |
| cdc-3 | II | CJB111 | 100 | 0/674 |
| cdc-21 | | CJB111 | 100 | 0/674 |
| 1999 | IV | CJB111 | 100 | 0/674 |
| D136C | III | CJB111 | 98.4 | 11/674 |
| nem316 | | CJB111 | 98.4 | 11/674 |
| COH31 | | CJB111 | 98.2 | 12/674 |
| 5408 | VIII | CJB111 | 98.4 | 11/674 |
| DK21 | II | dk21 | 100 | |
| cdc-1 | | dk21 | 99.3 | 5/682 |
| cdc-5 | | dk21 | 100 | 0/682 |
| cdc-9 | | dk21 | 100 | 0/682 |
| cdc-26 | | dk21 | 100 | 0/432 gap di 250aa |
| cdc-41 | | dk21 | 100 | 0/682 |
| cdc-47 | | dk21 | 99.8 | 1/669 gap di 13aa |
| 1169 | NT | dk21 | 100 | 0/682 |
| 515 | Ia | 515 | 100 | |
| DK1 | | 515 | 100 | 0/675 |
| DK8 | | 515 | 100 | 0/675 |
| Davis | | 515 | 100 | 0/675 |
| 2986 | | 515 | 100 | 0/675 |
| 5551 | | 515 | 100 | 0/675 |
| cdc-11 | | 515 | 100 | 0/675 |
| cdc-13 | | 515 | 100 | 0/675 |
| cdc-22 | | 515 | 100 | 0/675 |
| cdc-31 | | 515 | 100 | 0/675 |
| cdc-25 | | 515 | 100% + 11aa | 0/675 + 11aa |
| 2110 | V | 515 | 100 | 0/675 |
| 2210 | IV | 515 | 100 | 0/675 |
| CJB111 | V | CJB111 | 100 | |
| 5364 | | CJB111 | 100 | 0/674 |
| cdc-4 | | CJB111 | 100 | 0/674 |
| cdc-6 | | CJB111 | 100 | 0/674 |
| cdc-12 | | CJB111 | 100 | 0/674 |
| cdc-16 | | CJB111 | 100 | 0/674 |
| cdc-20 | | CJB111 | 100 | 0/674 |
| cdc-23 | | CJB111 | 100 | 0/674 |
| cdc-28 | | CJB111 | 100 | 0/674 |
| cdc-30 | | CJB111 | 100 | 0/674 |
| cdc-35 | | CJB111 | 100 | 0/674 |
| cdc-40 | | CJB111 | 100 | 0/674 |
| 7357b- | Ib | CJB111 | 100 | 0/674 |
| 5518 | | CJB111 | 100 | 0/674 |
| cdc-2 | | CJB111 | 100 | 0/674 |
| cdc-36 | | CJB111 | 100 | 0/674 |
| cdc-3 | II | CJB111 | 100 | 0/674 |
| cdc-21 | | CJB111 | 100 | 0/674 |
| 1999 | IV | CJB111 | 100 | 0/674 |
| D136C | III | CJB111 | 98.4 | 11/674 |
| nem316 | | CJB111 | 98.4 | 11/674 |
| COH31 | | CJB111 | 98.2 | 12/674 |
| 5408 | VIII | CJB111 | 98.4 | 11/674 |
| DK21 | II | dk21 | 100 | |
| cdc-1 | | dk21 | 99.3 | 5/682 |
| cdc-5 | | dk21 | 100 | 0/682 |
| cdc-9 | | dk21 | 100 | 0/682 |
| cdc-26 | | dk21 | 100 | 0/432 gap di 250aa |
| cdc-41 | | dk21 | 100 | 0/682 |
| cdc-47 | | dk21 | 99.8 | 1/669 gap di 13aa |
| 1169 | NT | dk21 | 100 | 0/682 |
| 2603 | V | 2603 | 100 | |
| 1998 | III | 2603 | 100 | 0/705 |
| cdc-17 | | 2603 | 100 | 0/705 |
| cdc-19 | | 2603 | 100 | 0/705 |
| cdc-44 | | 2603 | 99.9 | 1/705 |
| cdc-46 | | 2603 | 99.9 | 1/705 |
| cdc-48 | | 2603 | 97.3 | 20/705 |
| cdc-49 | | 2603 | 100 | 0/705 |
| cdc-27 | | 2603 | 99.9 | 1/705 |
| 18RS21 | II | 2603 | 100 | 0/705 |
| 3050 | | 2603 | 100 | 0/705 |
| 2141 | | 2603 | 100 | 0/705 |
| 2928 | VII | 2603 | 99.9 | 1/705 |
| H36B | Ib | H36B | 100 | |
| 2129 | | H36B | 99.7 | 2/693 |
| 5401 | II | H36B | 99.9 | 1/693 |
| cdc-39 | | H36B | 99.7 | 2/693 |
| 2274 | IV | H36B | 99.9 | 1/693 |
| CJB110 | NT | cjb110 | 100 | 0/704aa |
| 090 | Ia | cjb110 | 100 | 0/475 + stop codon |
| cdc-10 | | cjb110 | 100 | 0/704aa |

| | | GBS 59 |
|---|---|---|
| GBS strains | Type | PCR |
| A909 | Ia | — |
| cdc-7 | III | — |
| cdc-8 | III | — |
| cdc-14 | III | — |
| cdc-15 | III | — |
| cdc-18 | III | — |
| cdc-34 | III | — |
| COH1 | III | — |
| M732 | III | — |
| M781 | III | — |
| 5376 | III | — |
| 5435 | III | — |
| 2177 | Ib | — |
| SMU071 | VIII | — |
| JM9130013 | VIII | — |

Members of the same allelic family will typically have 75% sequence identity or more (e.g. 80%, 85%, 90%, 91%, 92%, 93%, 94%, 95%, 96%, 97%, 98%, 99%, 99.5%). More preferably, members of the same allelic family will have 97% or more sequence identity or more. Even more preferably, members of the same allelic family will exhibit immunological cross-reactivity. As used herein, the term “cross-reactivity” refers to the characteristic of an antigen to elicit an immune response effective against more than one strain of GBS (e.g., heterologous GBS strains). According to this classification, two new variants of GBS59 are distinguishable: one represented by GBS59 from strain CJB110 (i.e., GBS59^{CJB110}) and the second represented by GBS59, encoded from strain DK21 (i.e., GBS59^{DK21}). As shown in FIG. 240, GBS59^{CJB110} amino acid sequence is 72% identical to that of GBS59²⁶⁰³ and GBS59^{DK21} shares 71% sequence identity with GBS59^{CJB111}.

Accordingly, immunogenic composition of the invention for the treatment or prophylaxis of GBS infections may be further improved by combining GBS59 polypeptides from different allelic families or fragments thereof, in order to increase strain coverage. In some embodiments, when no cross-reactivity is detected between two or more allelic families, the combination will preferably include representative polypeptides from each allelic family. In further embodiments, when GBS59 polypeptides from different allelic families cross-react, the immunogenic composition of the invention may include only one representative polypeptide. In other embodiments, when an allelic family contains GBS59 polypeptides from specifically virulent strains, the immunogenic composition of the invention will preferably contain representative antigens from that allelic family.

Immunizations with GBS59 polypeptides of the invention are discussed further in the Examples.

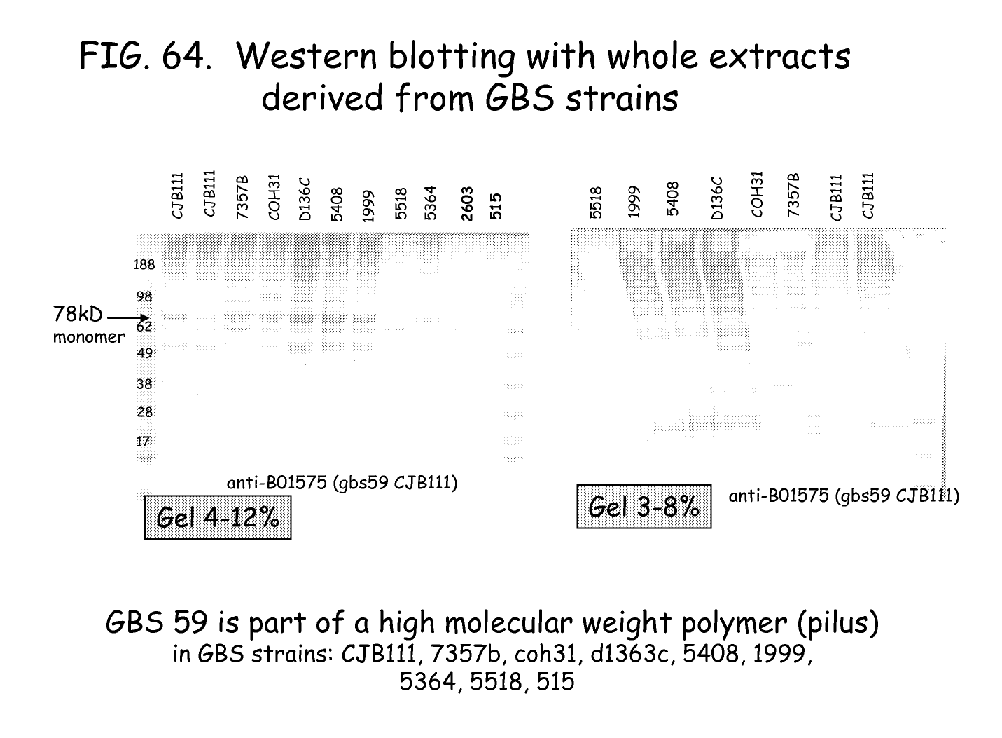

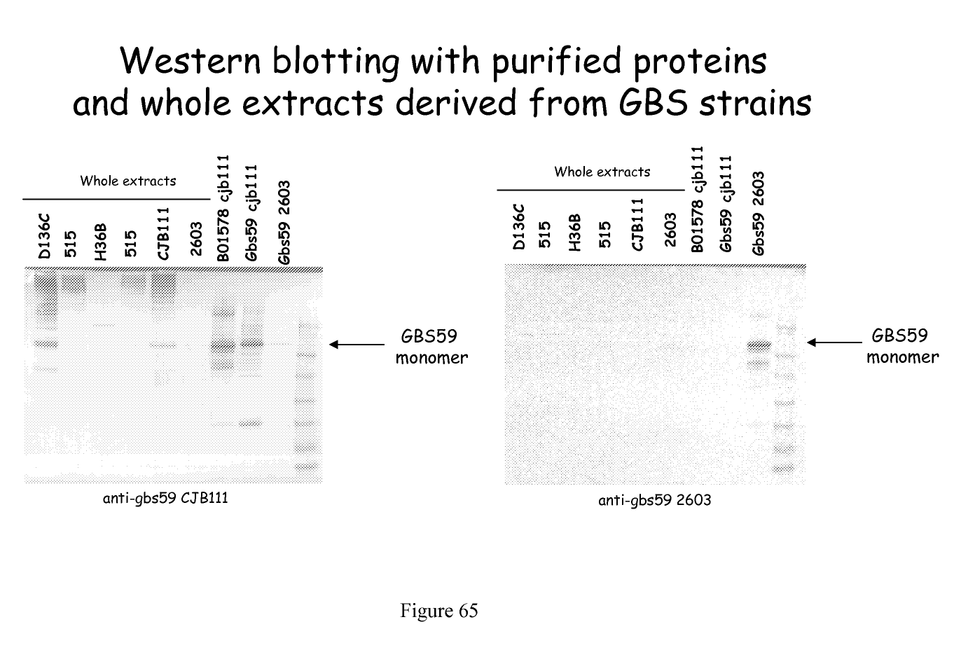

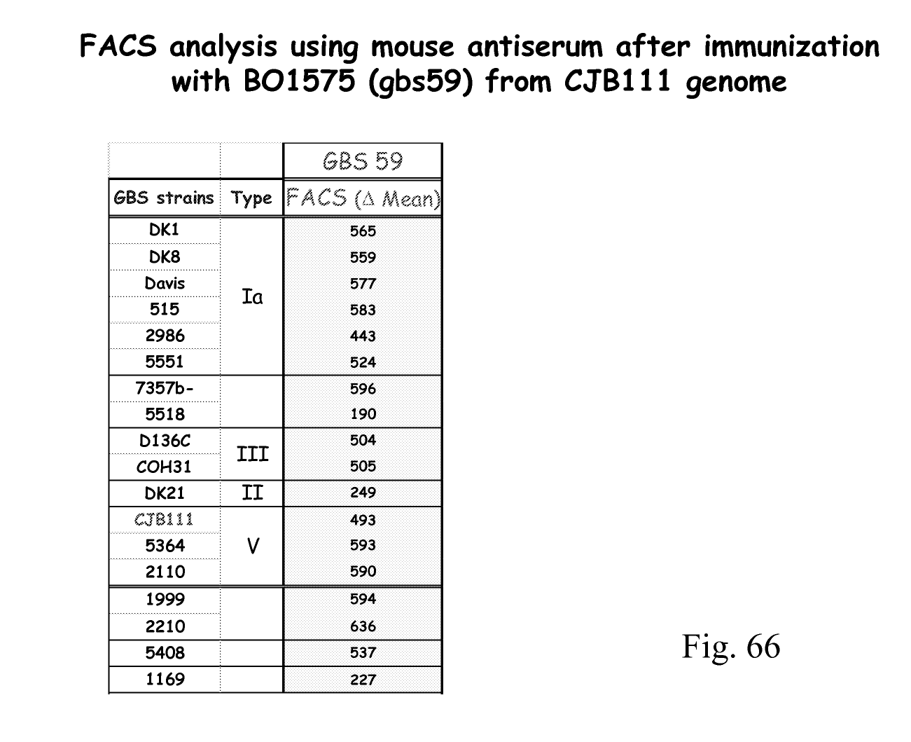

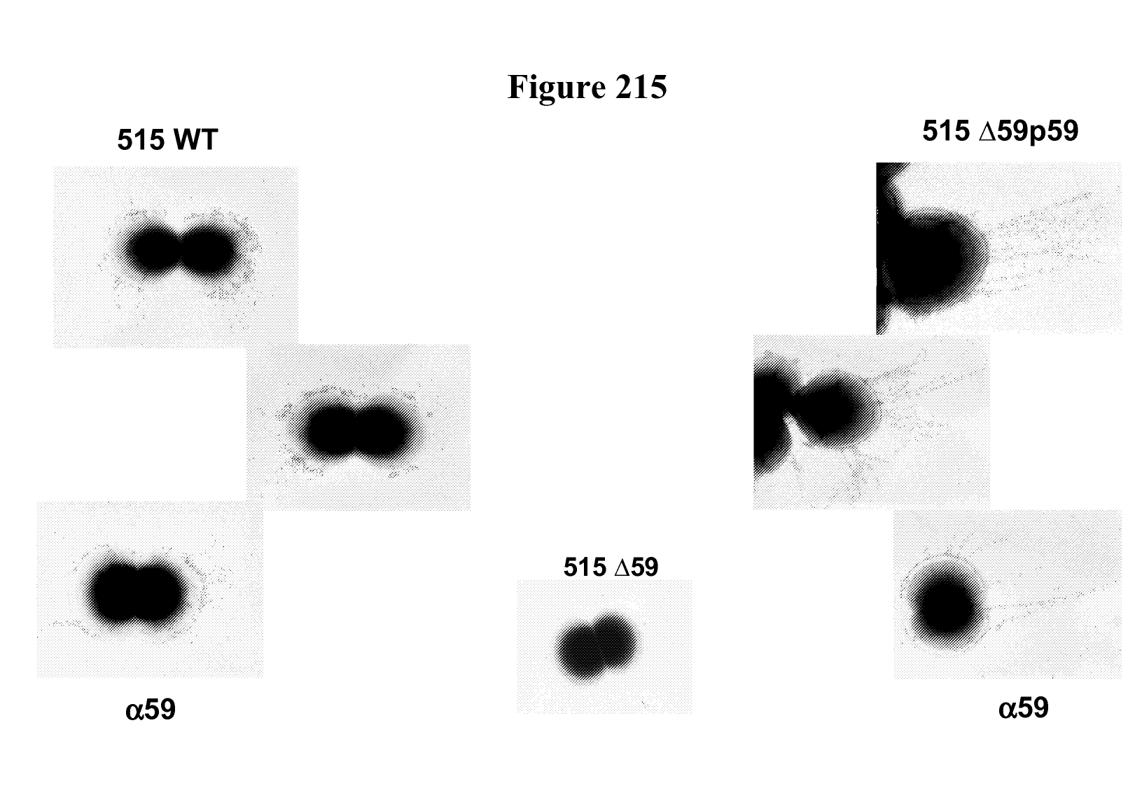

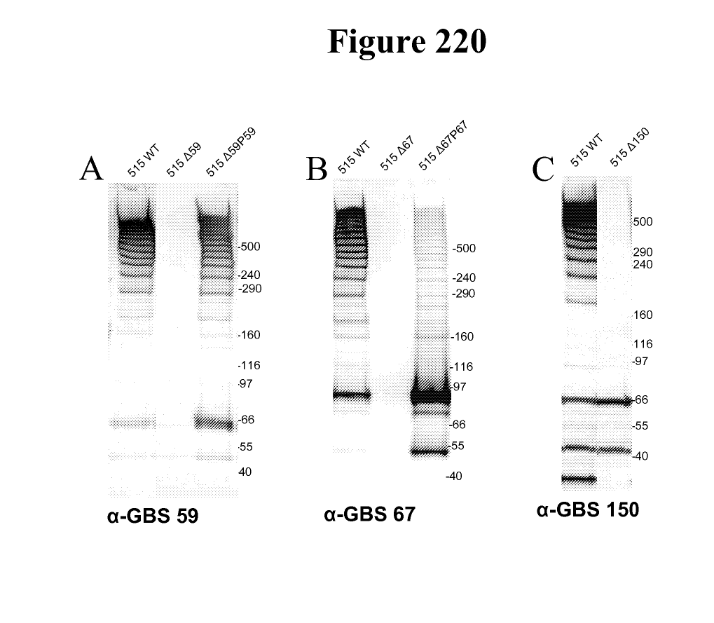

The gene encoding GBS 59 has been identified in a high number of GBS isolates; the GBS 59 gene was detected in 31 of 40 GBS isolates tested (77.5%). The GBS 59 protein also appears to be present as part of a pilus in whole extracts derived from GBS strains. FIG. 64 shows detection of high molecular weight GBS 59 polymers in whole extracts of GBS strains CJB111, 7357B, COH31, D1363C, 5408, 1999, 5364, 5518, and 515 using antiserum raised against GBS 59 of GBS strain CJB111. FIG. 65 also shows detection of these high molecular weight GBS 59 polymers in whole extracts of GBS strains D136C, 515, and CJB111 with anti-GBS 59 antiserum. (See also FIG. 220A for detection of GBS 59 high molecular weight polymers in strain 515.) FIG. 65 confirms the presence of different isoforms of GBS 59. Antisera raised against two different GBS 59 isoforms results in different patterns of immunoreactivity depending on the GBS strain origin of the whole extract. FIG. 65 further shows detection of GBS 59 monomers in purified GBS 59 preparations. GBS 59 is also highly expressed on the surface of GBS strains. GBS 59 was detected on the surface of GBS strains CJB111, DK1, DK8, Davis, 515, 2986, 5551, 1169, and 7357B by FACS analysis using mouse antiserum raised against GBS 59 of GBS CJB111. FACS analysis did not detect surface expression of GBS 59 in GBS strains SMU071, JM9130013, and COH1, which do not contain a GBS 59 gene. (See FIG. 66.) Further confirmation that GBS 59 is expressed on the surface of GBS is detection of GBS 59 by immuno-electron microscopy on the surface of GBS strain 515 bacteria. See FIG. 215.

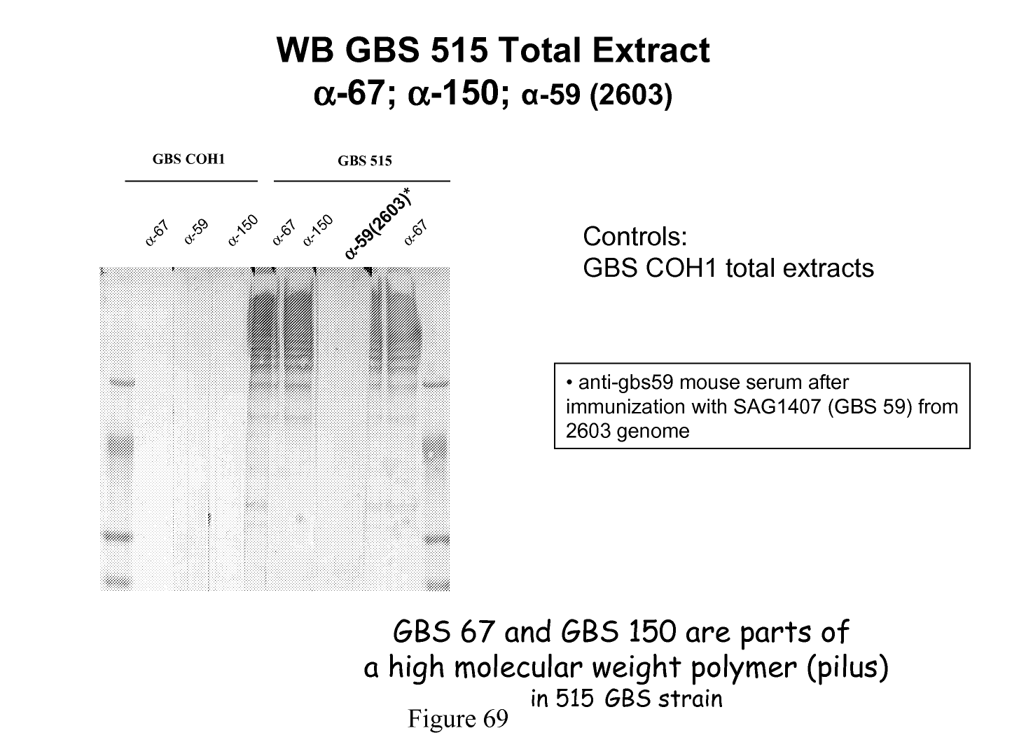

GBS 67 and GBS 150 also appear to be included in high molecular weight structures, or pili. FIG. 69 shows that anti-GBS 67 and anti-GBS 150 immunoreact with high molecular weight structures in whole GBS strain 515 extracts. (See also FIG. 220B and C.) It is also notable in FIG. 69 that the anti-GBS 59 antisera, raised in a mouse following immunization with GBS 59 of GBS strain 2603, does not cross-hybridize with GBS 59 in GBS strain 515. GBS 59 of GBS stain 515 is of a different isotype than GBS 59 of GBS stain 2603. See FIG. 63, which illustrates that the homology of these two GBS 59 polypeptides is 48%, and FIG. 65, which confirms that GBS 59 antisera raised against GBS strain 2603 does not cross-hybridize with GBS 59 of GBS strain 515.

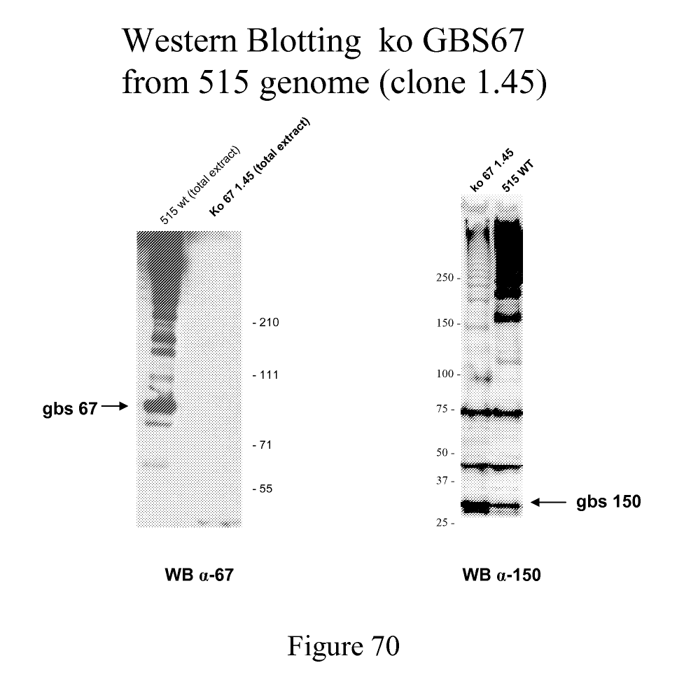

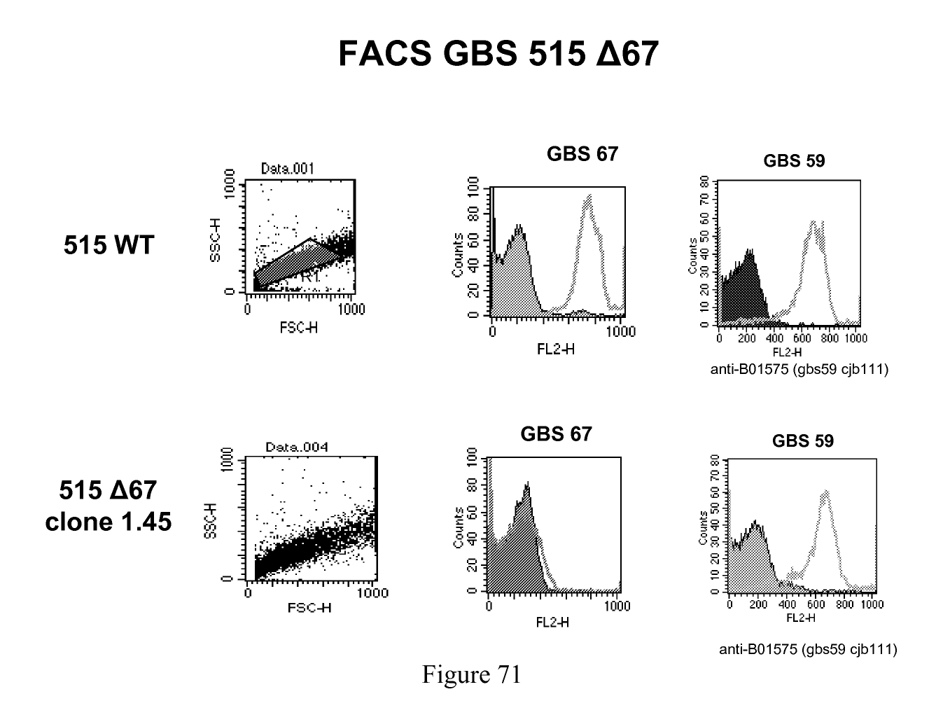

Formation of pili containing GBS 150 does not appear to require GBS 67 expression. FIG. 70 provides Western blots showing that higher molecular weight structures in GBS strain 515 total extracts immunoreact with anti-GBS 67 and anti-GBS 150 antiserum. In a GBS strain 515 lacking GBS 67 expression, anti-GBS 67 antiserum no longer immunoreacts with polypeptides in total extracts, while anti-GBS 150 antiserum is still able to cross-hybridize with high molecular weight structures.

Likewise, formation of pili containing GBS 59 does not appear to require GBS 67 expression. As expected, FACS detects GBS 67 cell surface expression on wildtype GBS strain 515, but not GBS strain 515 cells knocked out for GBS 67. FACS analysis using anti-GBS 59 antisera, however, detects GBS 59 expression on both the wildtype GBS strain 515 cells and the GBS strain 515 cells knocked out for GBS 67. Thus, GBS 59 cell surface expression is detected on GBS stain 515 cells regardless of GBS 67 expression.

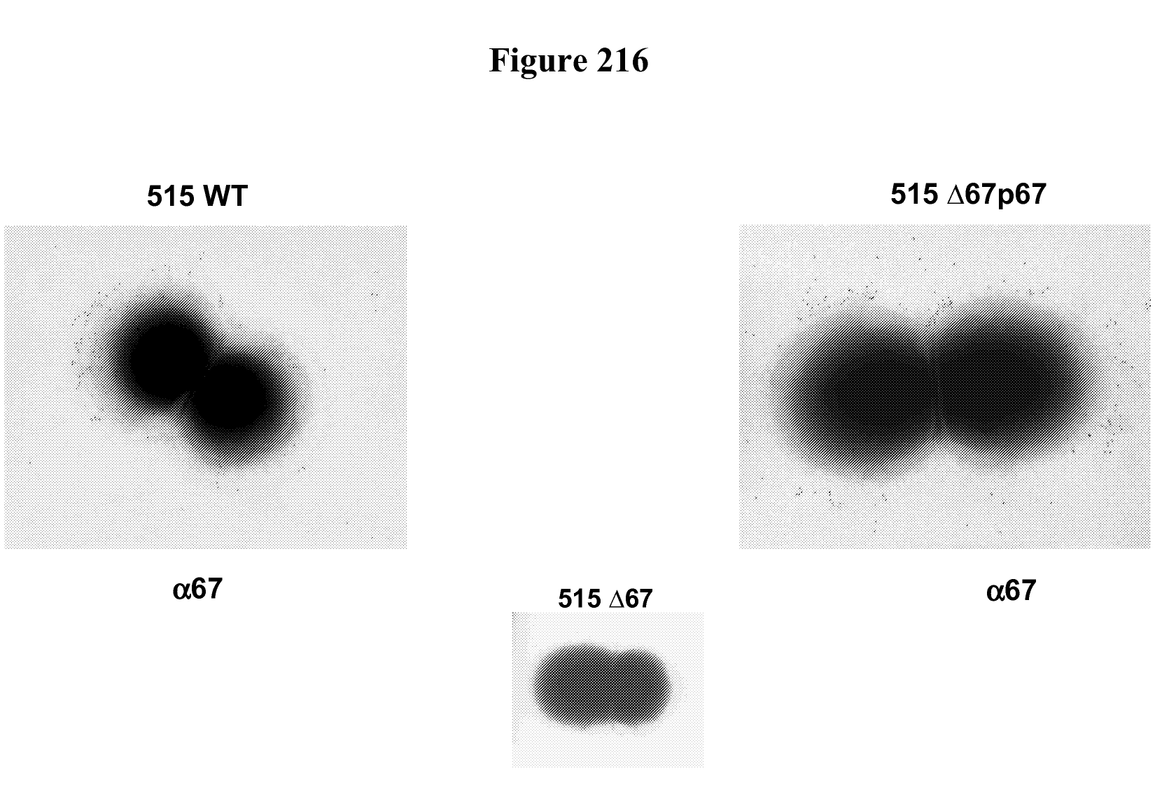

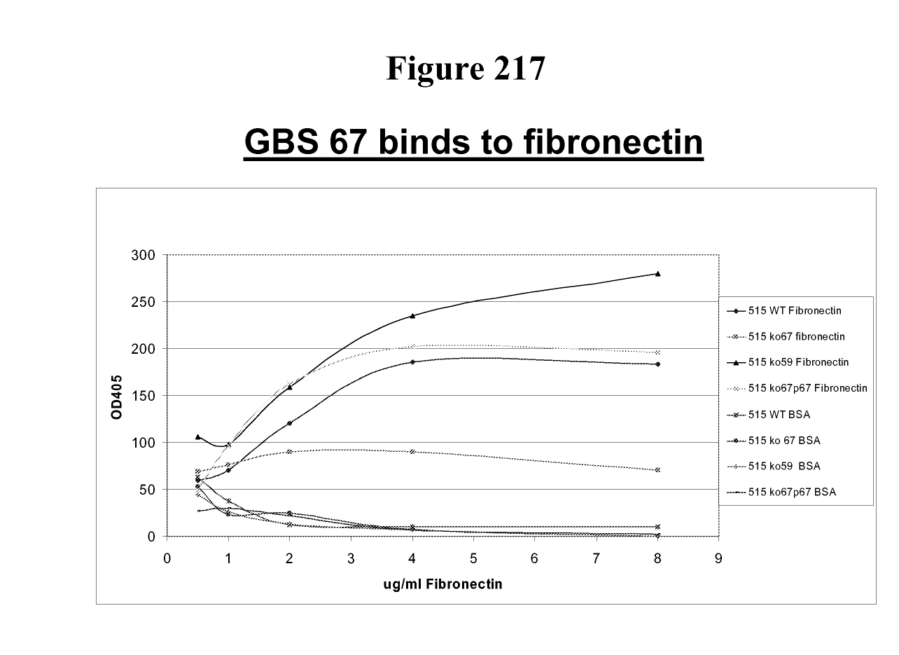

GBS 67, while present in pili, appears to be localized around the surface of GBS strain 515 cells. See the immuno-electron micrographs presented in FIG. 216. GBS 67 binds to fibronectin. See FIG. 217.

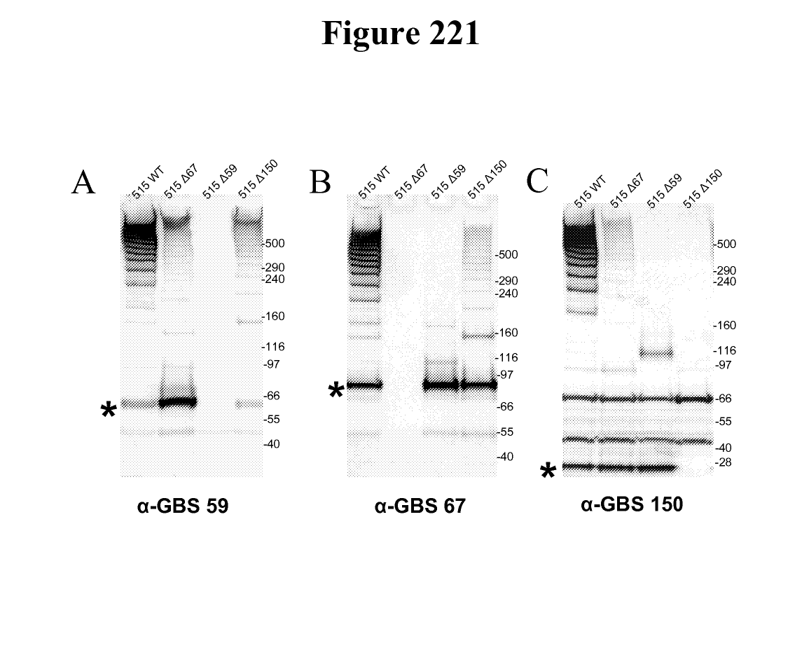

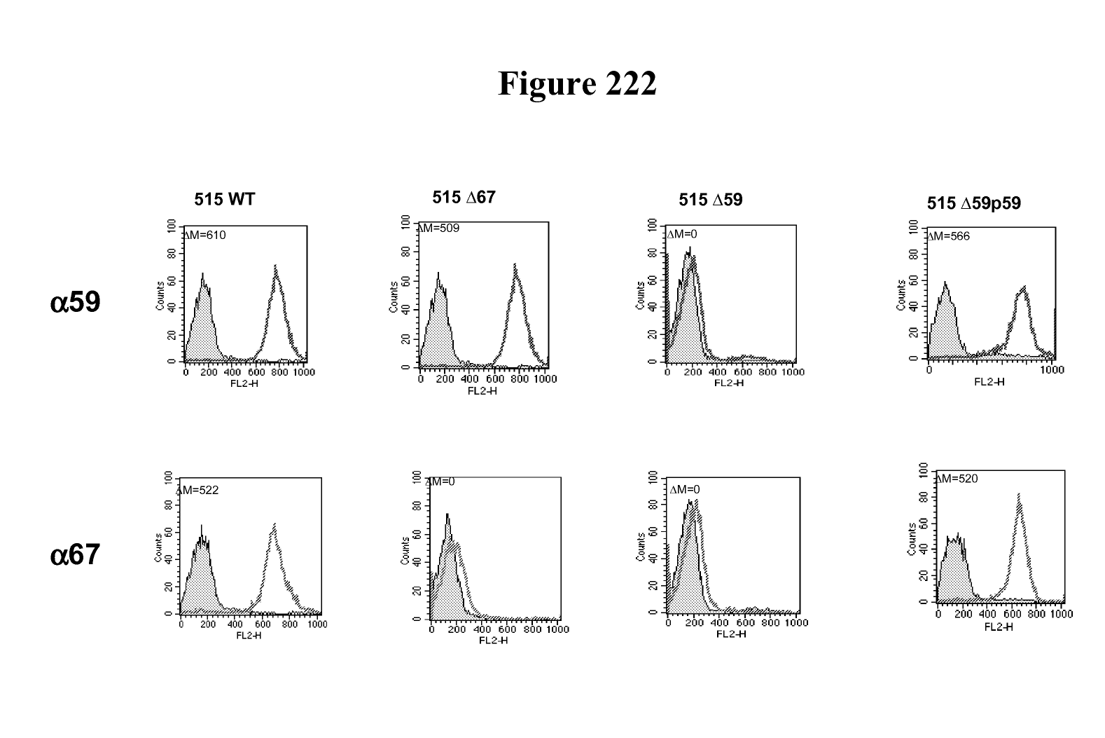

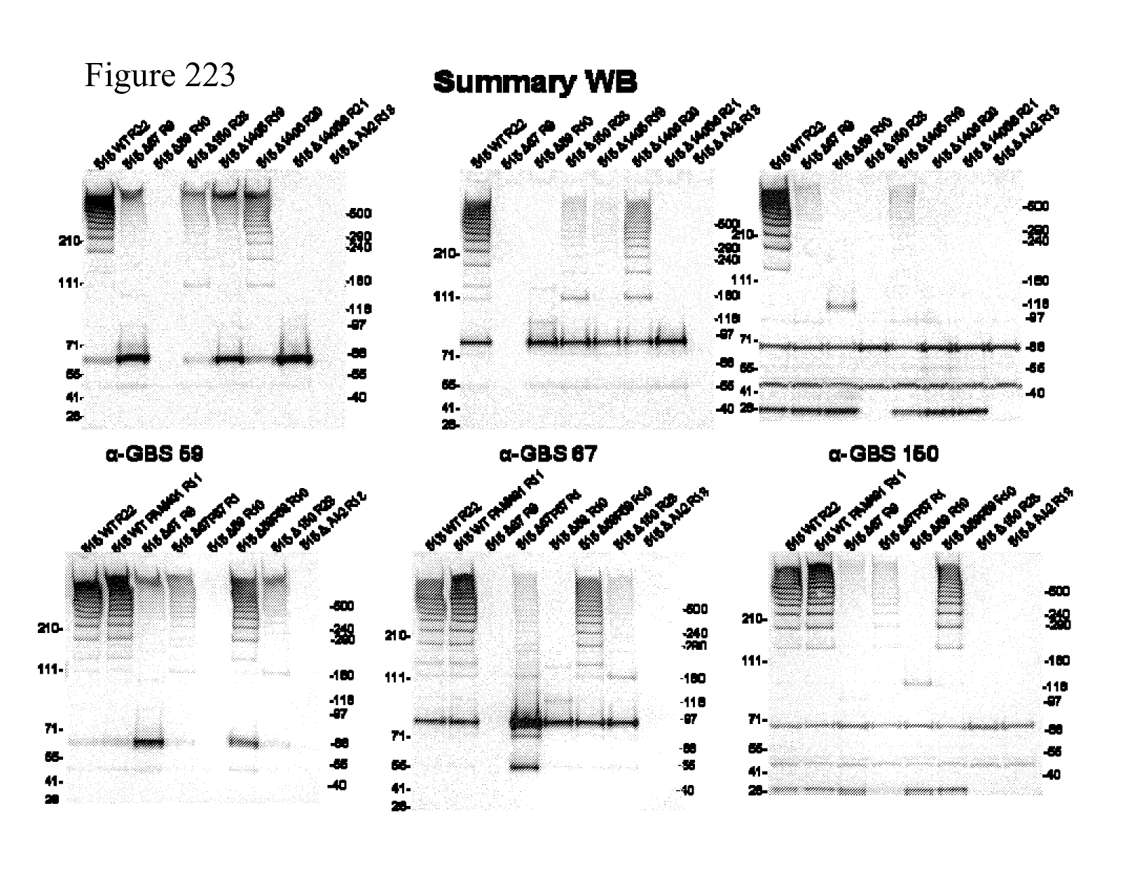

Formation of pili encoded by GBS AI-2 does require expression of GBS 59. Deletion of GBS 59 from strain 515 bacteria eliminates detection of high molecular weight structures by antibodies that bind to GBS 59 (FIG. 221A, lane 3), GBS 67 (FIG. 221B, lane 3), and GBS 150 (FIG. 221C, lane 3). By contrast, Western blot analysis of 515 bacteria with a deletion of the GBS 67 gene detects high molecular weight structures using GBS 59 (FIG. 221A, lane 2) and GBS 150 (FIG. 221C, lane 2) antisera. Similarly, Western blot analysis of 515 bacteria with a deletion of the GBS 150 gene detects high molecular weight structures using GBS 59 (FIG. 221A, lane 4) and GBS 67 (FIG. 221B, lane 4). See also FIG. 223, which provides Western blots of each of the 515 strains interrogated with antibodies for GBS 59, GBS 67, and GBS 150. FACS analysis of strain 515 bacteria deleted for either GBS 59 or GBS 67 confirms these results. See FIG. 222, which shows that only deletion of GBS 59 abolishes surface expression of both GBS 59 and GBS 67.

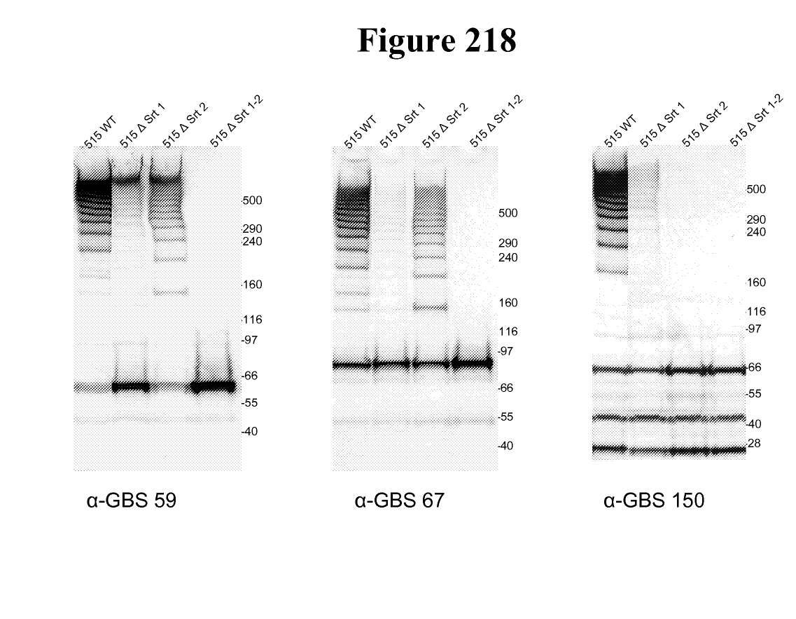

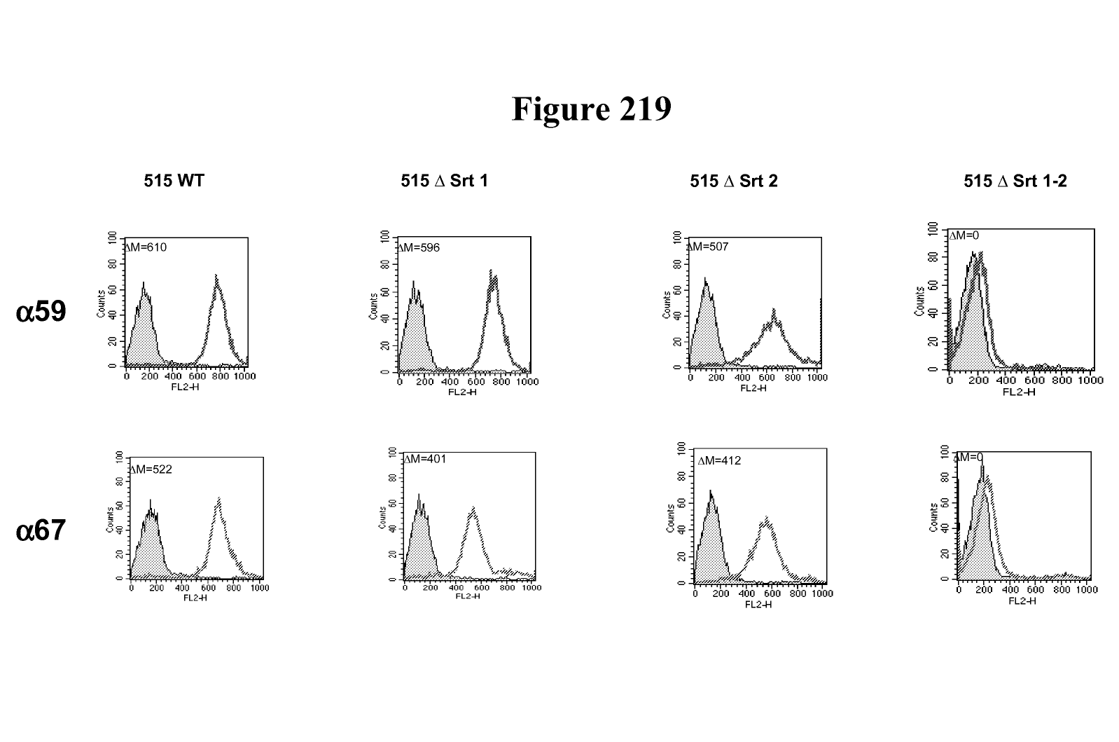

Formation of pili encoded by GBS AI-2 also requires expression of both GBS adhesin island-2 encoded sortases. See FIG. 218, which provides Western blot analysis of strain 515 bacteria lacking Srt1, Srt2, or both Srt1 and Srt2. Only deletion of both Srt1 and Srt2 abolishes pilus assembly as detected by antibodies that cross-hybridize with each of GBS 59, GBS 67 and GBS 150. The results of the Western blot analysis were verified by FACS, which provided similar results. See FIG. 219.

As shown in FIG. 4, two of the GBS strain isolates (COH 1 and A909) do not appear to contain homologues to the surface proteins GBS 59 and GBS 67. For these two strains, the percentages shown in FIG. 4 are amino acid identity to the COH1 protein). Notwithstanding the difference in the surface protein lengths for these two strains, AI-2 within these sequences still contains two sortase proteins and three LPXTG containing surface proteins, as well as a signal peptidase sequence leading into the first surface protein. One of the surface proteins in this variant of AI-2, spb1, has previously been identified as a potential adhesion protein. (See Adderson et al., Infection and Immunity (2003) 71(12):6857-6863). Alternatively, because of the lack of GBS 59 and GBS 67 sequences, this variant of AI-2 may be a third type of AI (Adhesin Island-3, AI-3, or GBS AI-3).

More than one AI surface protein may be present in the oligomeric, pilus-like structures of the invention. For example, GBS 59 and GBS 67 may be incorporated into an oligomeric structure. Alternatively, GBS 59 and GBS 150 may be incorporated into an oligomeric structure, or GBS 59, GBS 150 and GBS 67 may be incorporated into an oligomeric structure.

In another embodiment, the invention includes compositions comprising two or more AI surface proteins. The composition may include surface proteins from the same adhesin island. For example, the composition may include two or more GBS AI-2 surface proteins, such as GBS 59, GBS 67 and GBS 150. The surface proteins may be isolated from Gram positive bacteria or they may be produced recombinantly.

### GAS Adhesin Islands

Applicants have identified at least six different GAS Adhesin Islands. These adhesion islands are thought to encode surface proteins which are important in the bacteria's virulence, and Applicants have obtained the first electron micrographs revealing the presence of these adhesin island proteins in hyperoligomeric pilus structures on the surface of Group A Streptococcus.

Group A Streptococcus is a human specific pathogen which causes a wide variety of diseases ranging from pharyngitis and impetigo through life threatening invasive disease and necrotizing fasciitis. In addition, post-streptococcal autoimmune responses are still a major cause of cardiac pathology in children.

Group A Streptococcal infection of its human host can generally occur in three phases. The first phase involves attachment and/or invasion of the bacteria into host tissue and multiplication of the bacteria within the extracellular spaces. Generally this attachment phase begins in the throat or the skin. The deeper the tissue level infected, the more severe the damage that can be caused. In the second stage of infection, the bacteria secretes a soluble toxin that diffuses into the surrounding tissue or even systemically through the vasculature. This toxin binds to susceptible host cell receptors and triggers inappropriate immune responses by these host cells, resulting in pathology. Because the toxin can diffuse throughout the host, the necrosis directly caused by the GAS toxins may be physically located in sites distant from the bacterial infection. The final phase of GAS infection can occur long after the original bacteria have been cleared from the host system. At this stage, the host's previous immune response to the GAS bacteria due to cross reactivity between epitopes of a GAS surface protein, M, and host tissues, such as the heart. A general review of GAS infection can be found in Principles of Bacterial Pathogenesis, Groisman ed., Chapter 15 (2001).

In order to prevent the pathogenic effects associated with the later stages of GAS infection, an effective vaccine against GAS will preferably facilitate host elimination of the bacteria during the initial attachment and invasion stage.

Isolates of Group A Streptococcus are historically classified according to the M surface protein described above. The M protein is surface exposed trypsin-sensitive protein generally comprising two polypeptide chains complexed in an alpha helical formation. The carboxyl terminus is anchored in the cytoplasmic membrane and is highly conserved among all group A streptococci. The amino terminus, which extend through the cell wall to the cell surface, is responsible for the antigenic variability observed among the 80 or more serotypes of M proteins.

A second layer of classification is based on a variable, trypsin-resistant surface antigen, commonly referred to as the T-antigen. Decades of epidemiology based on M and T serological typing have been central to studies on the biological diversity and disease causing potential of Group A Streptococci. While the M-protein component and its inherent variability have been extensively characterized, even after five decades of study, there is still very little known about the structure and variability of T-antigens. Antisera to define T types is commercially available from several sources, including Sevapharma (sevapharma.cz/en).

The gene coding for one form of T-antigen, T-type 6, from an M6 strain of GAS (D741) has been cloned and characterized and maps to an approximately 11 kb highly variable pathogenicity island. Schneewind et al., J Bacteriol. (1990) 172(6):3310-3317. This island is known as the Fibronectin-binding, Collagen-binding T-antigen (FCT) region because it contains, in addition to the T6 coding gene (tee6), members of a family of genes coding for Extra Cellular Matrix (ECM) binding proteins. Bessen et al., Infection & Immunity (2002) 70(3):1159-1167. Several of the protein products of this gene family have been shown to directly bind either fibronectin and/or collagen. See Hanski et al., Infection & Immunity (1992) 60(12):5119-5125; Talay et al., Infection & Immunity (1992(60(9):3837-3844; Jaffe et al. (1996) 21(2):373-384; Rocha et al., Adv Exp Med Biol. (1997) 418:737-739; Kreikemeyer et al., J Biol Chem (2004) 279(16):15850-15859; Podbielski et al., Mol. Microbiol. (1999) 31(4):1051-64; and Kreikemeyer et al., Int. J. Med Microbiol (2004) 294(2-3):177-88. In some cases direct evidence for a role of these proteins in adhesion and invasion has been obtained.

Applicants raised antiserum against a recombinant product of the tee6 gene and used it to explore the expression of T6 in M6 strain 2724. In immunoblot of mutanolysin extracts of this strain, the antiserum recognized, in addition to a band corresponding to the predicted molecular mass of the product, very high molecular weight ladders ranging in mobility from about 100 kDa to beyond the resolution of the 3-8% gradient gels used.

This pattern of high molecular weight products is similar to that observed in immunoblots of the protein components of the pili identified in Streptococcus agalactiae (described above) and previously in Corynebacterium diphtheriae. Electron microscopy of strain M6_{—}2724 with antisera specific for the product of tee6 revealed abundant surface staining and long pilus like structures extending up to 700 nanometers from the bacterial surface, revealing that the T6 protein, one of the antigens recognized in the original Lancefield serotyping system, is located within a GAS Adhesin Island (GAS AI-1) and forms long covalently linked pilus structures.

Applicants have identified at least six different Group A Streptococcus Adhesin Islands. While these GAS AI sequences can be identified in numerous M types, Applicants have surprisingly discovered a correlation between the four main pilus subunits from the four different GAS AI types and specific T classifications. While other trypsin-resistant surface exposed proteins are likely also implicated in the T classification designations, the discovery of the role of the GAS adhesin islands (and the associated hyper-oligomeric pilus like structures) in T classification and GAS serotype variance has important implications for prevention and treatment of GAS infections. Applicants have identified protein components within each of the GAS adhesin islands which are associated with the pilus formation. These proteins are believed to be involved in the bacteria's initial adherence mechanisms. Immunological recognition of these proteins may allow the host immune response to slow or prevent the bacteria's transition into the more pathogenic later stages of infection.

In addition, Applicants have discovered that the GBS pili structures appear to be implicated in the formation of biofilms (populations of bacteria growing on a surface, often enclosed in an exopolysaccharide matrix). Biofilms are generally associated with bacterial resistance, as antibiotic treatments and host immune response are frequently unable to eradicate all of the bacteria components of the biofilm. Direction of a host immune response against surface proteins exposed during the first steps of bacterial attachment (i.e., before complete biofilm formation) is preferable.

The invention therefore provides for improved immunogenic compositions against GAS infection which may target GAS bacteria during their initial attachment efforts to the host epithelial cells and may provide protection against a wide range of GAS serotypes. The immunogenic compositions of the invention include GAS AI surface proteins which may be formulated in an oligomeric, or hyperoligomeric (pilus) form. The invention also includes combinations of GAS AI surface proteins. Combinations of GAS AI surface proteins may be selected from the same adhesin island or they may be selected from different GAS adhesin islands.

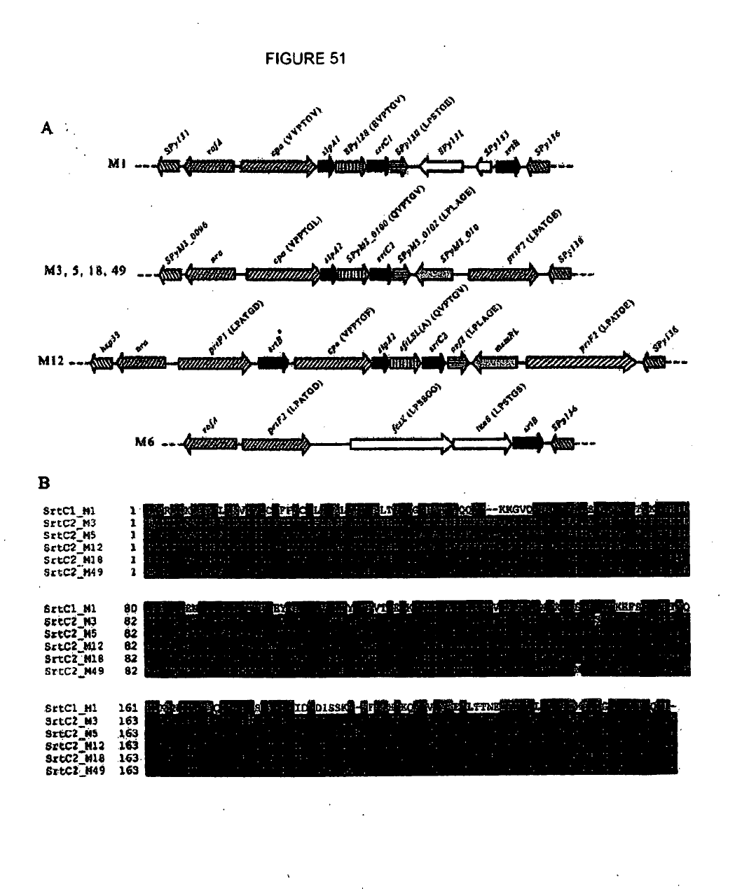

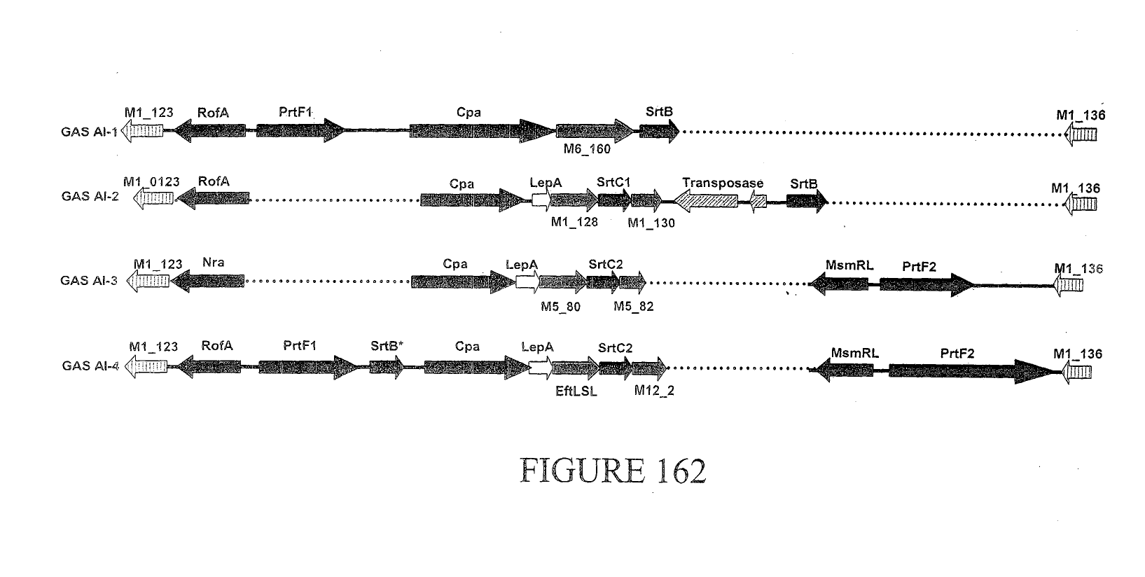

While there is surprising variability in the number and sequence of the GAS AI components across isolates, GAS AI sequences may be generally characterized as Type 1, Type 2, Type 3, and Type 4, depending on the number and type of sortase sequence within the island and the percentage identity of other proteins within the island. Schematics of the GAS adhesin islands are set forth in FIG. 51A and FIG. 162. In all strains identified so far, the adhesin island region is flanked by highly conserved open reading frames M1_{—}123 and M1_{—}36. Between three and five genes in each GAS adhesin island code for ECM binding adhesin proteins containing LPXTG motifs.

GAS Adhesin Island 1

As discussed above, Applicants have identified adhesin islands, “GAS Adhesin Island 1” or “GAS AI-1,” within the genome Group A Streptococcus serotypes and isolates. GAS AI-1 comprises a series of approximately five open reading frames encoding for a collection of amino acid sequences comprising surface proteins and sortases (“GAS AI-1 proteins”). GAS AI-1 preferably comprises surface proteins, a srtB sortase, and a rofA divergently transcribed transcriptional regulator. GAS AI-1 surface proteins may include a fibronectin binding protein, a collagen adhesion protein and a fimbrial structural subunit. Preferably, each of these GAS AI-1 surface proteins includes an LPXTG sortase substrate motif, such as LPXTG (SEQ ID NO:122) or LPXSG (SEQ ID NO:134) (conservative replacement of threonine with serine). Specifically, GAS AI-1 includes open reading frames encoding for two or more (i.e., 2, 3, 4 or 5) of M6_Spy0157, M6_Spy0158, M6_Spy0159, M6_Spy0160, M6_Spy0161.

Applicants have also identified open reading frames encoding fimbrial structural subunits in other GAS bacteria harbouring an AI-1. These open reading frames encode fimbrial structural subunits CDC SS 410_fimbrial, ISS3650_fimbrial, and DSM2071_fimbrial. A GAS AI-1 may comprise a polynucleotide encoding any one of CDC SS 410_fimbrial, ISS3650_fimbrial, and DSM2071_fimbrial.

As discussed above, the hyper-oligomeric pilus structure of GAS AI-1 appears to be responsible for the T-antigen type 6 classification, and GAS AI-1 corresponds to the FCT region previously identified for tee6. As in GAS AI-1, the tee6 FCT region includes open reading frames encoding for a collagen adhesion protein (cpa, capsular polysaccharide adhesion) and a fibronectin binding protein (prtF1). Immunoblots of tee6, a GAS AI-1 fimbrial structural subunit corresponding to M6_Spy160, reveal high molecular weight structures indicative of the hyper-oligomeric pilus structures Immunoblots with antiserum specific for Cpa also recognize a high molecular weight ladder structure, indicating Cpa involvement in the GAS AI-1 pilus structure or formation. In EM photos of GAS bacteria, Cpa antiserum reveals abundant staining on the surface of the bacteria and occasional gold particles extended from the surface of the bacteria. In contrast, immunoblots with antiserum specific for PrtF1 recognize only a single molecular species with electrophoretic mobility corresponding to its predicted molecular mass, indicating that PrtF1 may not be associated with the oligomeric pilus structure. A preferred immunogenic composition of the invention comprises a GAS AI-1 surface protein which may be formulated or purified in an oligomeric (pilus) form. In a preferred embodiment, the oligomeric form is a hyperoligomer. Another preferred immunogenic composition of the invention comprises a GAS AI-1 surface protein which has been isolated in an oligomeric (pilus) form. The oligomer or hyperoligomeric pilus structures comprising the GAS AI-1 surface proteins may be purified or otherwise formulate for use in immunogenic compositions.

One or more of the GAS AI-1 open reading frame polynucleotide sequences may be replaced by a polynucleotide sequence coding for a fragment of the replaced ORF. Alternatively, one or more of the GAS AI-1 open reading frames may be replaced by a sequence having sequence homology to the replaced ORF.

One or more of the GAS AI-1 surface protein sequences typically include an LPXTG motif (e.g., SEQ ID NO:122) or other sortase substrate motif.

The LPXTG sortase substrate motif of a GAS AI surface protein may be generally represented by the formula XXXXG, wherein X at amino acid position 1 is an L, a V, an E, or a Q, wherein X at amino acid position 2 is a P if X at amino acid position 1 is an L, wherein X at amino acid position 2 is a V if X at amino acid position 1 is a E or a Q, wherein X at amino acid position 2 is a V or a P if X at amino acid position 1 is a V, wherein X at amino acid position 3 is any amino acid residue, wherein X at amino acid position 4 is a T if X at amino acid position 1 is a V, E, or Q, and wherein X at amino acid position 4 is a T, S, or A if X at amino acid position 1 is an L. Some examples of LPXTG motifs present in GAS AI surface proteins include LPSXG (SEQ ID NO:134), VVXTG (SEQ ID NO:135), EVXTG (SEQ ID NO:136), VPXTG (SEQ ID NO:137), QVXTG (SEQ ID NO:138), LPXAG (SEQ ID NO:139), QVPTG (SEQ ID NO:140), and FPXTG (SEQ ID NO:141).

The GAS AI surface proteins of the invention may affect the ability of the GAS bacteria to adhere to and invade epithelial cells. AI surface proteins may also affect the ability of GAS to translocate through an epithelial cell layer. Preferably, one or more GAS AI surface proteins are capable of binding to or otherwise associating with an epithelial cell surface. GAS AI surface proteins may also be able to bind to or associate with fibrinogen, fibronectin, or collagen.

The GAS AI-1 sortase proteins are predicted to be involved in the secretion and anchoring of the LPXTG containing surface proteins. GAS AI-1 may encode for at least one surface protein. Alternatively, GAS AI-1 may encode for at least two surface exposed proteins and at least one sortase. Preferably, GAS AI-1 encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

GAS AI-1 preferably includes a srtB sortase. GAS srtB sortases may preferably anchor surface proteins with an LPSTG motif (SEQ ID NO:166), particularly where the motif is followed by a serine.

In one embodiment, the invention includes a composition comprising oligomeric, pilus-like structures comprising a GAS AI-1 surface protein such as M6_Spy0157, M6_Spy0159, M6_Spy0160, CDC SS 410_fimbrial, ISS3650_fimbrial, or DSM2071_fimbrial. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a GAS Adhesin Island protein in oligomeric form, preferably in a hyperoligomeric form. In one embodiment, the invention comprises a composition comprising one or more GAS Adhesin Island 1 (“GAS AI-1”) proteins and one or more GAS Adhesin Island 2 (“GAS AI-2”), GAS Adhesin Island 3 (“GAS AI-3”), GAS Adhesin Island 4 (“GAS AI-4”), GAS Adhesin Island 5 (“GAS AI-5”), or GAS Adhesin Island 6 (“GAS AI-6”) proteins, wherein one or more of the GAS Adhesin Island proteins is in the form of an oligomer, preferably in a hyperoligomeric form.

In addition to the open reading frames encoding the GAS AI-1 proteins, GAS AI-1 may also include a divergently transcribed transcriptional regulator such as RofA (i.e., the transcriptional regulator is located near or adjacent to the AI protein open reading frames, but it transcribed in the opposite direction).

### GAS Adhesin Island 2

A second adhesin island, “GAS Adhesin Island 2” or “GAS AI-2” has also been identified in Group A Streptococcus serotypes and isolates. GAS AI-2 comprises a series of approximately eight open reading frames encoding for a collection of amino acid sequences comprising surface proteins and sortases (“GAS AI-2 proteins”). Specifically, GAS AI-2 includes open reading frames encoding for two or more (i.e., 2, 3, 4, 5, 6, 7, or 8) of GAS 15, Spy0127, GAS16, GAS17, GAS18, Spy0131, Spy0133, and GAS20.

A preferred immunogenic composition of the invention comprises a GAS AI-2 surface protein which may be formulated or purified in an oligomeric (pilus) form. In a preferred embodiment, the oligomeric form is a hyperoligomer. Another preferred immunogenic composition of the invention comprises a GAS AI-2 surface protein which has been isolated in an oligomeric (pilus) form. The oligomer or hyperoligomeric pilus structures comprising the GAS AI-2 surface proteins may be purified or otherwise formulate for use in immunogenic compositions.

One or more of the GAS AI-2 open reading frame polynucleotide sequences may be replaced by a polynucleotide sequence coding for a fragment of the replaced ORF. Alternatively, one or more of the GAS AI-2 open reading frames may be replaced by a sequence having sequence homology to the replaced ORF.

One or more of the GAS AI-2 surface protein sequences typically include an LPXTG motif (e.g., SEQ ID NO:122) or other sortase substrate motif. The AI surface proteins of the invention may affect the ability of the GAS bacteria to adhere to and invade epithelial cells. AI surface proteins may also affect the ability of GAS to translocate through an epithelial cell layer. Preferably, one or more AI surface proteins are capable of binding to or otherwise associating with an epithelial cell surface. AI surface proteins may also be able to bind to or associate with fibrinogen, fibronectin, or collagen.

The GAS AI-2 sortase proteins are predicted to be involved in the secretion and anchoring of the LPXTG containing surface proteins. GAS AI-2 may encode for at least one surface protein. Alternatively, GAS AI-2 may encode for at least two surface exposed proteins and at least one sortase. Preferably, GAS AI-2 encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising an AI surface protein such as GAS15, GAS16, or GAS18. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine amino acid residue.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a GAS Adhesin Island protein in oligomeric form, preferably in a hyperoligomeric form. In one embodiment, the invention comprises a composition comprising one or more GAS Adhesin Island 2 (“GAS AI-2”) proteins and one or more GAS Adhesin Island 1 (“GAS AI-1”), GAS Adhesin Island 3 (“GAS AI-3”), GAS Adhesin Island 4 (“GAS AI-4”) proteins, GAS Adhesin Island 5 (“GAS AI-5”), or GAS Adhesin Island 6 (“GAS AI-6”) proteins, wherein one or more of the Adhesin Island proteins is in the form of an oligomer, preferably in a hyperoligomeric form.

In addition to the open reading frames encoding the GAS AI-2 proteins, GAS AI-2 may also include a divergently transcribed transcriptional regulator such as rofA (i.e., the transcriptional regulator is located near or adjacent to the AI protein open reading frames, but it transcribed in the opposite direction).

GAS Adhesin Island 3

A third adhesin island, “GAS Adhesin Island 3” or “GAS AI-3” has also been identified in several Group A Streptococcus serotypes and isolates. GAS AI-3 comprises a series of approximately seven open reading frames encoding for a collection of amino acid sequences comprising surface proteins and sortases (“GAS AI-3 proteins”). Specifically, GAS AI-3 includes open reading frames encoding for two or more (i.e., 2, 3, 4, 5, 6, or 7) of SpyM3_{—}0098, SpyM3_{—}0099, SpyM3_{—}0100, SpyM3_{—}0101, SpyM3_{—}0102, SpyM3_{—}0103, SpyM3_{—}0104, SPs0100, SPs0101, SPs0102, SPs0103, SPs0104, SPs0105, SPs0106, orf78, orf79, orf80, orf81, orf82, orf83, orf84, spyM18_{—}0126, spyM18_{—}0127, spyM18_{—}0128, spyM18_{—}0129, spyM18_{—}0130, spyM18_{—}0131, spyM18_{—}0132, SpyoM01000156, SpyoM01000155, SpyoM01000154, SpyoM01000153, SpyoM01000152, SpyoM01000151, SpyoM01000150, and SpyoM01000149. In one embodiment, GAS AI-3 includes open reading frames encoding for two or more (i.e., 2, 3, 4, 5, 6, or 7) of SpyM3_{—}0098, SpyM3_{—}0099, SpyM3_{—}0100, SpyM3_{—}0101, SpyM3_{—}0102, SpyM3_{—}0103, and SpyM3_{—}0104. In another embodiment, GAS AI-3 includes open reading frames encoding for two or more (i.e., 2, 3, 4, 5, 6, or 7) of SPs0100, SPs0101, SPs0102, SPs0103, SPs0104, SPs0105, and SPs0106. In a further embodiment, GAS AI-3 includes open reading frames encoding for two or more (i.e., 2, 3, 4, 5, 6, or 7) of orf78, orf79, orf80, orf81, orf82, orf83, and orf84. In yet another embodiment, GAS AI-3 includes open reading frames encoding for two or more (i.e., 2, 3, 4, 5, 6, or 7) of spyM18_{—}0126, spyM18_{—}0127, spyM18_{—}0128, spyM18_{—}0129, spyM18_{—}0130, spyM18_{—}0131, and spyM18_{—}0132. In yet another embodiment, GAS AI-3 includes open reading frames encoding for two or more (i.e., 2, 3, 4, 5, 6, or 7) of SpyoM01000156, SpyoM01000155, SpyoM01000154, SpyoM01000153, SpyoM01000152, SpyoM01000151, SpyoM01000150, and SpyoM01000149.

Applicants have also identified open reading frames encoding fimbrial structural subunits in other GAS bacteria harbouring an AI-3. These open reading frames encode fimbrial structural subunits ISS3040_fimbrial, ISS3776_fimbrial, and ISS4959_fimbrial. A GAS AI-3 may comprise a polynucleotide encoding any one of ISS3040_fimbrial, ISS3776_fimbrial, and ISS4959_fimbrial.

One or more of the GAS AI-3 open reading frame polynucleotide sequences may be replaced by a polynucleotide sequence coding for a fragment of the replaced ORF. Alternatively, one or more of the GAS AI-3 open reading frames may be replaced by a sequence having sequence homology to the replaced ORF.

A preferred immunogenic composition of the invention comprises a GAS AI-3 surface protein which may be formulated or purified in an oligomeric (pilus) form. In a preferred embodiment, the oligomeric form is a hyperoligomer. Another preferred immunogenic composition of the invention comprises a GAS AI-3 surface protein which has been isolated in an oligomeric (pilus) form. The oligomer or hyperoligomeric pilus structures comprising the GAS AI-3 surface proteins may be purified or otherwise formulate for use in immunogenic compositions.

One or more of the GAS AI-3 surface protein sequences typically include an LPXTG motif (e.g., SEQ ID NO:122) or other sortase substrate motif. The AI surface proteins of the invention may affect the ability of the GAS bacteria to adhere to and invade epithelial cells. AI surface proteins may also affect the ability of GAS to translocate through an epithelial cell layer. Preferably, one or more AI surface proteins are capable of binding to or otherwise associating with an epithelial cell surface. AI surface proteins may also be able to bind to or associate with fibrinogen, fibronectin, or collagen.

The GAS AI-3 sortase proteins are predicted to be involved in the secretion and anchoring of the LPXTG containing surface proteins. GAS AI-3 may encode for at least one surface protein. Alternatively, GAS AI-3 may encode for at least two surface exposed proteins and at least one sortase. Preferably, GAS AI-3 encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine or alanine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

The invention includes a composition comprising oligomeric, pilus-like structures comprising an AI surface protein such as SpyM3_{—}0098, SpyM3_{—}0100, SpyM3_{—}0102, SpyM3_{—}0104, SPs0100, SPs0102, SPs0104, SPs0106, orf78, orf80, orf82, orf84, spyM18_{—}0126, spyM18_{—}0128, spyM18_{—}0130, spyM18_{—}0132, SpyoM01000155, SpyoM01000153, SpyoM01000151, SpyoM01000149, ISS3040_fimbrial, ISS3776_fimbrial, and ISS4959_fimbrial. In one embodiment, the invention includes a composition comprising oligomeric, pilus-like structures comprising an AI surface protein such as SpyM3_{—}0098, SpyM3_{—}0100, SpyM3_{—}0102, and SpyM3_{—}0104. In another embodiment, the invention includes a composition comprising oligomeric, pilus-like structures comprising an AI surface protein such as SPs0100, SPs0102, SPs0104, and SPs0106. In another embodiment, the invention includes a composition comprising oligomeric, pilus-like structures comprising an AI surface protein such as orf78, orf80, orf82, and orf84. In yet another embodiment, the invention includes a composition comprising oligomeric, pilus-like structures comprising an AI surface protein such as spyM18_{—}0126, spyM18_{—}0128, spyM18_{—}0130, and spyM18_{—}0132. In a further embodiment, the invention includes a composition comprising oligomeric, pilus-like structures comprising an AI surface protein such as SpyoM01000155, SpyoM01000153, SpyoM01000151, and SpyoM01000149. In yet a further embodiment, the invention includes a composition comprising oligomeric, pilus-like structures comprising an AI surface protein such as ISS3040_fimbrial, ISS3776_fimbrial, and ISS4959_fimbrial. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine amino acid residue.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a GAS Adhesin Island protein in oligomeric form, preferably in a hyperoligomeric form. In one embodiment, the invention comprises a composition comprising one or more GAS Adhesin Island 3 (“GAS AI-3”) proteins and one or more GAS Adhesin Island 1 (“GAS AI-1”), GAS Adhesin Island 2 (“GAS AI-2”), GAS Adhesin Island 4 (“GAS AI-4”) proteins, GAS Adhesin Island 5 (“GAS AI-5”), or GAS Adhesin Island 6 (“GAS AI-6”) proteins, wherein one or more of the Adhesin Island proteins is in the form of an oligomer, preferably in a hyperoligomeric form.

In addition to the open reading frames encoding the GAS AI-3 proteins, GAS AI-3 may also include a transcriptional regulator such as Nra.

GAS Adhesin Island 4

A fourth adhesin island, “GAS Adhesin Island 4” or “GAS AI-4” has also been identified in Group A Streptococcus serotypes and isolates. GAS AI-4 comprises a series of approximately eight open reading frames encoding for a collection of amino acid sequences comprising surface proteins and sortases (“GAS AI-4 proteins”). Specifically, GAS AI-4 includes open reading frames encoding for two or more (i.e., 2, 3, 4, 5, 6, 7, or 8) of 19224134, 19224135, 19223136, 19223137, 19224138, 19224139, 19224140, and 19224141.

Applicants have also identified open reading frames encoding fimbrial structural subunits in other GAS bacteria harbouring an AI-4. These open reading frames encode fimbrial structural subunits 20010296_fimbrial, 20020069_fimbrial, CDC SS 635_fimbrial, ISS4883_fimbrial, and ISS4538_fimbrial. A GAS AI-4 may comprise a polynucleotide encoding any one of 20010296_fimbrial, 20020069_fimbrial, CDC SS 635_fimbrial, ISS4883_fimbrial, and ISS4538_fimbrial.

One or more of the GAS AI-4 open reading frame polynucleotide sequences may be replaced by a polynucleotide sequence coding for a fragment of the replaced ORF. Alternatively, one or more of the GAS AI-4 open reading frames may be replaced by a sequence having sequence homology to the replaced ORF.

A preferred immunogenic composition of the invention comprises a GAS AI-4 surface protein which may be formulated or purified in an oligomeric (pilus) form. In a preferred embodiment, the oligomeric form is a hyperoligomer. Another preferred immunogenic composition of the invention comprises a GAS AI-4 surface protein which has been isolated in an oligomeric (pilus) form. The oligomer or hyperoligomeric pilus structures comprising the GAS AI-4 surface proteins may be purified or otherwise formulate for use in immunogenic compositions.

One or more of the GAS AI-4 surface protein sequences typically include an LPXTG motif (e.g., SEQ ID NO:122) or other sortase substrate motif. The AI surface proteins of the invention may effect the ability of the GAS bacteria to adhere to and invade epithelial cells. AI surface proteins may also affect the ability of GAS to translocate through an epithelial cell layer. Preferably, one or more AI surface proteins are capable of binding to or otherwise associating with an epithelial cell surface. AI surface proteins may also be able to bind to or associate with fibrinogen, fibronectin, or collagen.

The GAS AI-4 sortase proteins are predicted to be involved in the secretion and anchoring of the LPXTG containing surface proteins. GAS AI-4 may encode for at least one surface protein. Alternatively, GAS AI-4 may encode for at least two surface exposed proteins and at least one sortase. Preferably, GAS AI-4 encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising an AI surface protein such as 19224134, 19224135, 19224137, 19224139, 19224141, 20010296_fimbrial, 20020069_fimbrial, CDC SS 635_fimbrial, ISS4883_fimbrial, and ISS4538_fimbrial. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine amino acid residue.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a GAS Adhesin Island protein in oligomeric form, preferably in a hyperoligomeric form. In one embodiment, the invention comprises a composition comprising one or more GAS Adhesin Island 4 (“GAS AI-4”) proteins and one or more GAS Adhesin Island 1 (“GAS AI-1”), GAS Adhesin Island 2 (“GAS AI-2”), GAS Adhesin Island 3 (“GAS AI-3”) proteins, GAS Adhesin Island 5 (“GAS AI-5”), or GAS Adhesin Island 6 (“GAS AI-6”) proteins, wherein one or more of the Adhesin Island proteins is in the form of an oligomer, preferably in a hyperoligomeric form.

In addition to the open reading frames encoding the GAS AI-4 proteins, GAS AI-4 may also include a divergently transcribed transcriptional regulator such as rofA (i.e., the transcriptional regulator is located near or adjacent to the AI protein open reading frames, but it transcribed in the opposite direction).

GAS Adhesin Island 5

A fifth adhesin island, “GAS Adhesin Island 5” or “GAS AI-5” has also been identified in Group A Streptococcus serotypes and isolates. GAS AI-5 comprises a series of approximately 10 open reading frames encoding for a collection of amino acid sequences comprising surface proteins and sortases (“GAS AI-5 proteins”). Specifically, GAS AI-5 includes open reading frames encoding for two or more (i.e., 2, 3, 4, 5, 6, 7, 8, 9, or 10) of MGAS10270_Spy0108, MGAS10270_Spy0109, MGAS10270_Spy0110, MGAS10270_Spy0111, MGAS10270_Spy0112, MGAS10270_Spy0113, MGAS10270_Spy0114, MGAS10270_Spy0115, MGAS10270_Spy0116, and MGAS10270_Spy0117.

One or more of the GAS AI-5 open reading frame polynucleotide sequences may be replaced by a polynucleotide sequence coding for a fragment of the replaced ORF. Alternatively, one or more of the GAS AI-5 open reading frames may be replaced by a sequence having sequence homology to the replaced ORF.

A preferred immunogenic composition of the invention comprises a GAS AI-5 surface protein which may be formulated or purified in an oligomeric (pilus) form. In a preferred embodiment, the oligomeric form is a hyperoligomer. Another preferred immunogenic composition of the invention comprises a GAS AI-5 surface protein which has been isolated in an oligomeric (pilus) form. The oligomer or hyperoligomeric pilus structures comprising the GAS AI-5 surface proteins may be purified or otherwise formulate for use in immunogenic compositions.

One or more of the GAS AI-5 surface protein sequences typically include an LPXTG motif (such as IPxTG (SEQ ID NO:133) or FPxTG (SEQ ID NO:141) or other sortase substrate motif. The AI surface proteins of the invention may effect the ability of the GAS bacteria to adhere to and invade epithelial cells. AI surface proteins may also affect the ability of GAS to translocate through an epithelial cell layer. Preferably, one or more AI surface proteins are capable of binding to or otherwise associating with an epithelial cell surface. AI surface proteins may also be able to bind to or associate with fibrinogen, fibronectin, or collagen.

The GAS AI-5 sortase proteins are predicted to be involved in the secretion and anchoring of the LPXTG containing surface proteins. GAS AI-5 may encode for at least one surface protein. Alternatively, GAS AI-5 may encode for at least two surface exposed proteins and at least one sortase. Preferably, GAS AI-5 encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising an AI surface protein such as MGAS10270_Spy0108, MGAS10270_Spy0109, MGAS10270_Spy0110, MGAS10270_Spy0111, MGAS10270_Spy0112, MGAS10270_Spy0113, MGAS10270_Spy0114, MGAS10270_Spy0115, MGAS10270_Spy0116, and MGAS10270_Spy0117. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine amino acid residue.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a GAS Adhesin Island protein in oligomeric form, preferably in a hyperoligomeric form. In one embodiment, the invention comprises a composition comprising one or more GAS Adhesin Island 5 (“GAS AI-5”) proteins and one or more GAS Adhesin Island 1 (“GAS AI-1”), GAS Adhesin Island 2 (“GAS AI-2”), GAS Adhesin Island 3 (“GAS AI-3”), GAS Adhesin Island 4 (“GAS AI-4”), or GAS Adhesin Island 6 (“GAS AI-6”) proteins, wherein one or more of the Adhesin Island proteins is in the form of an oligomer, preferably in a hyperoligomeric form.

In addition to the open reading frames encoding the GAS AI-5 proteins, GAS AI-5 may also include a divergently transcribed transcriptional regulator such as rofA (i.e., the transcriptional regulator is located near or adjacent to the AI protein open reading frames, but it transcribed in the opposite direction).

TABLE 54
AI-5 proteins in M2 (10270)

| | | amino | Entrez | Entrez | | LPXTG |
|---|---|---|---|---|---|---|
| identification | strand | acids | protein | gene | ORF | motif |
| 33 kDa chaperonin | − | 290 | 94989616 | 4063967 | MGAS10270_Spy0107 | |
| Transcriptional regulator | − | 530 | 94989617 | 4063968 | MGAS10270_Spy0108 | |
| RofA | | | | | | |
| hypothetical protein | + | 578 | 94989618 | 4063969 | MGAS10270_Spy0109 | IPxTG |
| MGAS10270_Spy0109 | | | | | | |
| hypothetical protein | + | 307 | 94989619 | 4063970 | MGAS10270_Spy0110 | IPxTG |
| MGAS10270_Spy0110 | | | | | | |
| Sortase | + | 305 | 94989620 | 4063971 | MGAS10270_Spy0111 | |
| Sortase | + | 283 | 94989621 | 4063972 | MGAS10270_Spy0112 | |
| Collagen adhesion protein | + | 890 | 94989622 | 4063973 | MGAS10270_Spy0113 | FPxTG |
| hypothetical protein | + | 53 | 94989623 | 4063974 | MGAS10270_Spy0114 | |
| MGAS10270_Spy0114 | | | | | | |
| Sortase | + | 111 | 94989624 | 4063975 | MGAS10270_Spy0115 | |
| Sortase | + | 285 | 94989625 | 4063091 | MGAS10270_Spy0116 | |
| Fibronectin-binding | + | 1715 | 94989626 | 4063092 | MGAS10270_Spy0117 | LPxTG |
| protein | | | | | | |
| hypothetical protein | − | 221 | 94989627 | 4063093 | MGAS10270_Spy0118 | |
| MGAS10270_Spy0118 | | | | | | |

GAS Adhesin Island 6

A sixth adhesin island, “GAS Adhesin Island 6” or “GAS AI-6” has also been identified in Group A Streptococcus serotypes and isolates. GAS AI-6 comprises a series of approximately 10 open reading frames encoding for a collection of amino acid sequences comprising surface proteins and sortases (“GAS AI-6 proteins”). Specifically, GAS AI-6 includes open reading frames encoding for two or more (i.e., 2, 3, 4, 5, 6, 7, or 8) of MGAS10750_Spy0113, MGAS10750_Spy0114, MGAS10750_Spy0115, MGAS10750_Spy0116, MGAS10750_Spy0117, MGAS10750_Spy0118, MGAS10750_Spy0119, and MGAS10750_Spy0120.

One or more of the GAS AI-6 open reading frame polynucleotide sequences may be replaced by a polynucleotide sequence coding for a fragment of the replaced ORF. Alternatively, one or more of the GAS AI-6 open reading frames may be replaced by a sequence having sequence homology to the replaced ORF.

A preferred immunogenic composition of the invention comprises a GAS AI-6 surface protein which may be formulated or purified in an oligomeric (pilus) form. In a preferred embodiment, the oligomeric form is a hyperoligomer. Another preferred immunogenic composition of the invention comprises a GAS AI-6 surface protein which has been isolated in an oligomeric (pilus) form. The oligomer or hyperoligomeric pilus structures comprising the GAS AI-6 surface proteins may be purified or otherwise formulate for use in immunogenic compositions.

One or more of the GAS AI-6 surface protein sequences typically include an LPXTG motif (such as LPXTG (SEQ ID NO:122), IPxTG (SEQ ID NO:133) or FPxTG (SEQ ID NO:141) or other sortase substrate motif. The AI surface proteins of the invention may effect the ability of the GAS bacteria to adhere to and invade epithelial cells. AI surface proteins may also affect the ability of GAS to translocate through an epithelial cell layer. Preferably, one or more AI surface proteins are capable of binding to or otherwise associating with an epithelial cell surface. AI surface proteins may also be able to bind to or associate with fibrinogen, fibronectin, or collagen.

The GAS AI-6 sortase proteins are predicted to be involved in the secretion and anchoring of the LPXTG containing surface proteins. GAS AI-6 may encode for at least one surface protein. Alternatively, GAS AI-6 may encode for at least two surface exposed proteins and at least one sortase. Preferably, GAS AI-6 encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising an AI surface protein such as MGAS10750_Spy0113, MGAS10750_Spy0114, MGAS10750_Spy0115, MGAS10750_Spy0116, MGAS10750_Spy0117, MGAS10750_Spy0118, MGAS10750_Spy0119, and MGAS10750_Spy0120. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine amino acid residue.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a GAS Adhesin Island protein in oligomeric form, preferably in a hyperoligomeric form. In one embodiment, the invention comprises a composition comprising one or more GAS Adhesin Island 6 (“GAS AI-6”) proteins and one or more GAS Adhesin Island 1 (“GAS AI-1”), GAS Adhesin Island 2 (“GAS AI-2”), GAS Adhesin Island 3 (“GAS AI-3”), GAS Adhesin Island 4 (“GAS AI-4”), or GAS Adhesin Island 5 (“GAS AI-5”) proteins, wherein one or more of the Adhesin Island proteins is in the form of an oligomer, preferably in a hyperoligomeric form.

In addition to the open reading frames encoding the GAS AI-6 proteins, GAS AI-6 may also include a divergently transcribed transcriptional regulator such as rofA (i.e., the transcriptional regulator is located near or adjacent to the AI protein open reading frames, but it transcribed in the opposite direction).

TABLE 55
AI-6 proteins in M4 (10750)

| | | amino | Entrez | Entrez | | sortase |
|---|---|---|---|---|---|---|
| identification | strand | acids | protein | gene | ORF | motif |
| 33 kDa chaperonin | − | 290 | 94993508 | 4067252 | MGAS10750_Spy0112 | |
| Transcriptional regulator | − | 530 | 94993509 | 4067253 | MGAS10750_Spy0113 | |
| RofA | | | | | | |
| Fibronectin-binding | + | 539 | 94993510 | 4067254 | MGAS10750_Spy0114 | LPxTG |
| protein | | | | | | |
| Fibronectin-binding | + | 1367 | 94993511 | 4067255 | MGAS10750_Spy0115 | FPxTG |
| protein | | | | | | |
| Cell wall surface anchor | + | 720 | 94993512 | 4067256 | MGAS10750_Spy0116 | IPxTG |
| family protein | | | | | | |
| Cell wall surface anchor | + | 281 | 94993513 | 4067257 | MGAS10750_Spy0117 | IPxTG |
| family protein | | | | | | |
| Sortase | + | 290 | 94993514 | 4066578 | MGAS10750_Spy0118 | |
| Sortase | + | 277 | 94993515 | 4066579 | MGAS10750_Spy0119 | |
| Sortase | + | 238 | 94993516 | 4066580 | MGAS10750_Spy0120 | |
| hypothetical protein | − | 219 | 94993517 | 4066581 | MGAS10750_Spy0121 | |
| MGAS10750_Spy0121 | | | | | | |

The oligomeric, pilus-like structures of the invention may be combined with one or more additional GAS proteins. In one embodiment, the oligomeric, pilus-like structures comprise one or more AI surface proteins in combination with a second GAS protein.

The oligomeric, pilus-like structures may be isolated or purified from bacterial cultures in which the bacteria express an AI surface protein. The invention therefore includes a method for manufacturing an oligomeric AI surface antigen comprising culturing a GAS bacterium that expresses the oligomeric AI protein and isolating the expressed oligomeric AI protein from the GAS bacteria. The AI protein may be collected from secretions into the supernatant or it may be purified from the bacterial surface. The method may further comprise purification of the expressed AI protein. Preferably, the AI protein is in a hyperoligomeric form.

The oligomeric, pilus-like structures may be isolated or purified from bacterial cultures overexpressing an AI surface protein. The invention therefore includes a method for manufacturing an oligomeric Adhesin Island surface antigen comprising culturing a GAS bacterium adapted for increased AI protein expression and isolation of the expressed oligomeric Adhesin Island protein from the GAS bacteria. The AI protein may be collected from secretions into the supernatant or it may be purified from the bacterial surface. The method may further comprise purification of the expressed Adhesin Island protein. Preferably, the Adhesin Island protein is in a hyperoligomeric form.

The GAS bacteria are preferably adapted to increase AI protein expression by at least two (e.g., 2, 3, 4, 5, 8, 10, 15, 20, 25, 30, 35, 40, 45, 50, 60, 70, 80, 90, 100, 125, 150 or 200) times wild type expression levels.

GAS bacteria may be adapted to increase AI protein expression by any means known in the art, including methods of increasing gene dosage and methods of gene upregulation. Such means include, for example, transformation of the GAS bacteria with a plasmid encoding the AI protein. The plasmid may include a strong promoter or it may include multiple copies of the sequence encoding the AI protein. Optionally, the sequence encoding the AI protein within the GAS bacterial genome may be deleted. Alternatively, or in addition, the promoter regulating the GAS Adhesin Island may be modified to increase expression.

The invention further includes GAS bacteria which have been adapted to produce increased levels of AI surface protein. In particular, the invention includes GAS bacteria which have been adapted to produce oligomeric or hyperoligomeric AI surface protein. In one embodiment, the Gram positive bacteria of the invention are inactivated or attenuated to permit in vivo delivery of the whole bacteria, with the AI surface protein exposed on its surface.

The invention further includes GAS bacteria which have been adapted to have increased levels of expressed AI protein incorporated in pili on their surface. The GAS bacteria may be adapted to have increased exposure of oligomeric or hyperoligomeric AI proteins on its surface by increasing expression levels of LepA polypeptide, or an equivalent signal peptidase, in the GAS bacteria. Applicants have shown that deletion of LepA in strain SF370 bacteria, which harbour a GAS AI-2, abolishes surface exposure of M and pili proteins on the GAS. Increased levels of LepA expression in GAS are expected to result in increased exposure of M and pili proteins on the surface of GAS. Increased expression of LepA in GAS may be achieved by any means known in the art, such as increasing gene dosage and methods of gene upregulation. The GAS bacteria adapted to have increased levels of LepA expression may additionally be adapted to express increased levels of at least one pili protein.

Alternatively, the AI proteins of the invention may be expressed on the surface of a non-pathogenic Gram positive bacteria, such as Streptococcus gordonii (See, e.g., Byrd et al., “Biological consequences of antigen and cytokine co-expression by recombinant Streptococcus gordonii vaccine vectors,” Vaccine (2002) 20:2197-2205) or Lactococcus lactis (See, e.g., Mannam et al., “Mucosal Vaccine Made from Live, Recombinant Lactococcus lactis Protects Mice against Pharyngeal Infection with Streptococcus pyogenes” Infection and Immunity (2004) 72(6):3444-3450). As used herein, non-pathogenic Gram positive bacteria refer to Gram positive bacteria which are compatible with a human host subject and are not associated with human pathogenesis. Preferably, the non-pathogenic bacteria are modified to express the AI surface protein in oligomeric, or hyper-oligomeric form. Sequences encoding for an AI surface protein and, optionally, an AI sortase, may be integrated into the non-pathogenic Gram positive bacterial genome or inserted into a plasmid. The non-pathogenic Gram positive bacteria may be inactivated or attenuated to facilitate in vivo delivery of the whole bacteria, with the AI surface protein exposed on its surface. Alternatively, the AI surface protein may be isolated or purified from a bacterial culture of the non-pathogenic Gram positive bacteria. For example, the AI surface protein may be isolated from cell extracts or culture supernatants. Alternatively, the AI surface protein may be isolated or purified from the surface of the non-pathogenic Gram positive bacteria.

The non-pathogenic Gram positive bacteria may be used to express any of the GAS Adhesin Island proteins described herein. The non-pathogenic Gram positive bacteria are transformed to express an Adhesin Island surface protein. Preferably, the non-pathogenic Gram positive bacteria also express at least one Adhesin Island sortase. The AI transformed non-pathogenic Gram positive bacteria of the invention may be used to prevent or treat infection with pathogenic GAS.

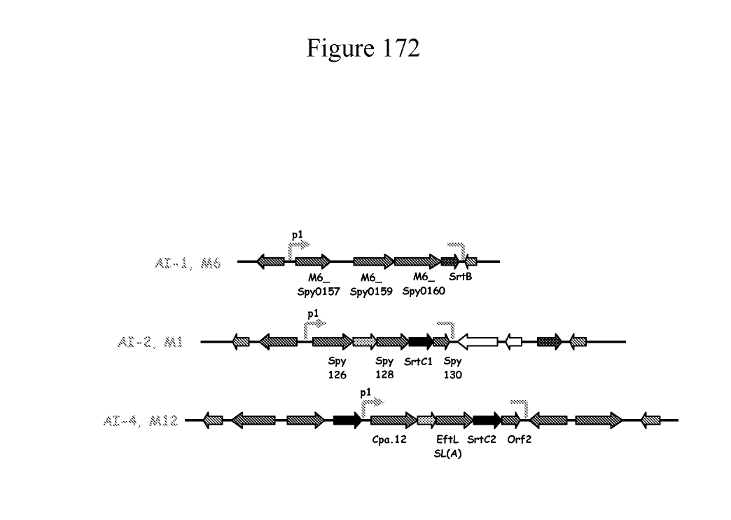

Applicants modified L. lactis to demonstrate that, like GBS polypeptides, it can express GAS AI polypeptides. L. lactis was transformed with pAM401 constructs encoding entire pili gene clusters of AI-1, AI-2, and AI-4 adhesin islands. Briefly, the pAM401 is a promoterless high-copy plasmid. The entire pili gene clusters of an M6 (AI-1), M1 (AI-2), and M12 (AI-4) bacteria were inserted into the pAM401 construct. The gene clusters were transcribed under the control their own (M6, M1, or M12) promoter or the GBS promoter that successfully initiated expression of the GBS AI-1 adhesin islands in L. lactis, described above. FIG. 172 provides a schematic depiction of GAS M6 (AI-1), M1 (AI-2), and M12 (AI-4) adhesin islands and indicates the portions of the adhesin island sequences inserted in the pAM401 construct.

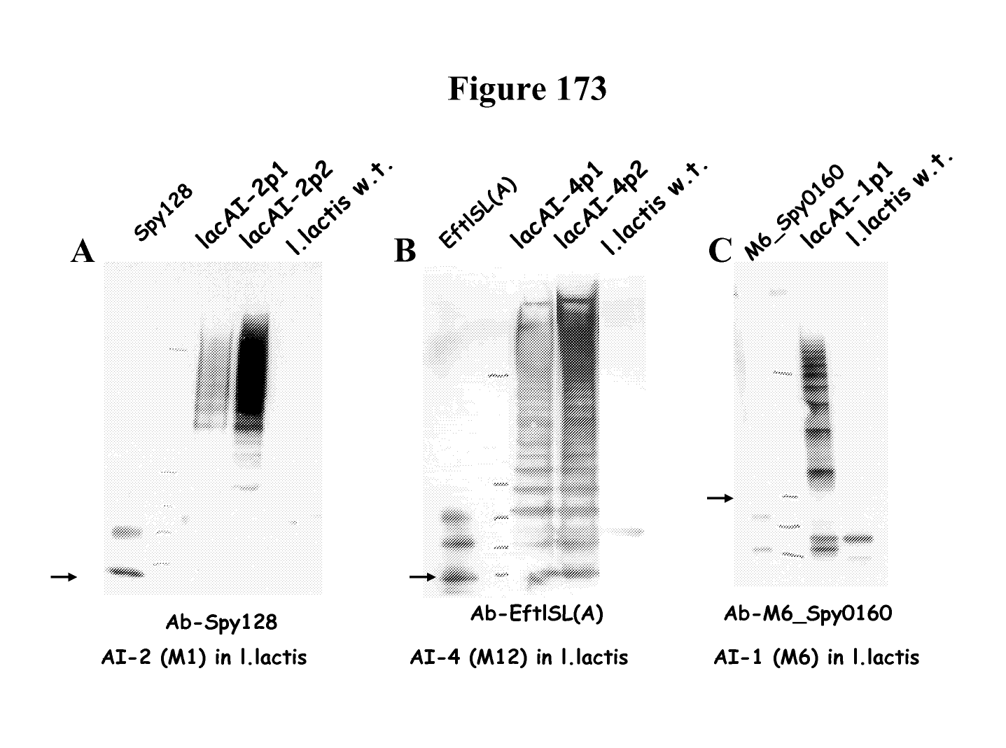

Each of the L. lactis transformed with one of the M6, M1, or M12 adhesin island gene clusters expressed high molecular weight structures that were immunoreactive with antibodies that bind to polypeptides present in their respective pili. FIGS. 173A-C provide results of Western blot analysis of surface protein-enriched extracts of L. lactis transformed with M6 (FIG. 173A), M1 (FIG. 173B), or M12 (FIG. 173C) adhesin island gene clusters using antibodies that bind to the fimbrial structural subunit encoded by each cluster. FIG. 173A at lanes 3 and 4 shows detection of high molecular structures in L. lactis transformed with an adhesin island pilus gene cluster from an M1 AI-2 using an antibody that binds to fimbrial structural subunit Spy0128. FIG. 173B at lanes 3 and 4 shows detection of high molecular weight structures in L. lactis transformed with an adhesin island pilus gene cluster from an M12 AI-4 using an antibody that binds to fimbrial structural subunit EftLSL.A. FIG. 173C at lane 3 shows detection of high molecular weight structures in L. lactis transformed with an adhesin island pilus gene cluster from an M6 AI-1 using an antibody that binds to fimbrial structural subunit M6_Spy0160. In FIGS. 173A-C, “p1” immediately following the notation of AI subtype indicates that the promoter present in the Adhesin Island is used to drive transcription of the adhesin island gene cluster and “p2” indicates that the promoter was the GBS promoter described above. Thus, it appears that L. lactis is capable of expressing the fimbrial structural subunits encoded by GAS adhesin islands in an oligomeric form.

Alternatively, the oligomeric, pilus-like structures may be produced recombinantly. If produced in a recombinant host cell system, the AI surface protein will preferably be expressed in coordination with the expression of one or more of the AI sortases of the invention. Such AI sortases will facilitate oligomeric or hyperoligomeric formation of the AI surface protein subunits.

S. pneumoniae from TIGR4 Adhesin Island

As discussed above, Applicants have identified adhesin islands within the genome of S. pneumoniae from TIGR4. The S. pneumoniae from TIGR4 Adhesin Island comprises a series of approximately seven open reading frames encoding for a collection of amino acid sequences comprising surface proteins and sortases. Specifically, the S. pneumoniae from TIGR4 AI proteins includes open reading frames encoding for two or more (i.e., 2, 3, 4, 5, 6, or 7) of SPO462, SPO463, SPO464, SPO465, SPO466, SPO467, and SPO468.

A preferred immunogenic composition of the invention comprises a S. pneumoniae from TIGR4 AI surface protein which may be formulated or purified in an oligomeric (pilus) form. In a preferred embodiment, the oligomeric form is a hyperoligomer. Another preferred immunogenic composition of the invention comprises a S. pneumoniae from TIGR4 AI surface protein which has been isolated in an oligomeric (pilus) form. The oligomer or hyperoligomer pilus structures comprising S. pneumoniae surface proteins may be purified or otherwise formulated for use in immunogenic compositions.

One or more of the S. pneumoniae from TIGR4 AI open reading frame polynucleotide sequences may be replaced by a polynucleotide sequence coding for a fragment of the replaced ORF. Alternatively, one or more of the S. pneumoniae from TIGR4 AI open reading frames may be replaced by a sequence having sequence homology to the replaced ORF.

One or more of the S. pneumoniae from TIGR4 AI surface protein sequences typically include an LPXTG motif (e.g., SEQ ID NO:122) or other sortase substrate motif.

The S. pneumoniae from TIGR4 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae from TIGR4 AI surface proteins are capable of binding to or otherwise associating with an epithelial cell surface. S. pneumoniae from TIGR4 AI surface proteins may also be able to bind to or associate with fibrinogen, fibronectin, or collagen.

The S. pneumoniae from TIGR4 AI sortase proteins are predicted to be involved in the secretion and anchoring of the LPXTG containing surface proteins. S. pneumoniae from TIGR4 AI may encode for at least one surface protein. Alternatively, S. pneumoniae from TIGR4 AI may encode for at least two surface exposed proteins and at least one sortase. Preferably, S. pneumoniae from TIGR4 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae from TIGR4 AI surface protein such as SPO462, SPO463, SPO464, or SPO465. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae from TIGR4 AI protein in oligomeric form, preferably in a hyperoligomeric form. In one embodiment, the invention comprises a composition comprising one or more S. pneumoniae from TIGR4 AI proteins and one or more S. pneumoniae strain 670 AI proteins, wherein one or more of the S. pneumoniae AI proteins is in the form of an oligomer, preferably in a hyperoligomeric form.

In addition to the open reading frames encoding the S. pneumoniae from TIGR4 AI proteins, S. pneumoniae from TIGR4 AI may also include a transcriptional regulator.

S. pneumoniae Strain 670 Adhesin Island

As discussed above, Applicants have identified adhesin islands within the genome of S. pneumoniae strain 670. The S. pneumoniae strain 670 Adhesin Island comprises a series of approximately seven open reading frames encoding for a collection of amino acid sequences comprising surface proteins and sortases. Specifically, the S. pneumoniae strain 670 AI proteins includes open reading frames encoding for two or more (i.e., 2, 3, 4, 5, 6, or 7) of orf1_{—}670, orf3_{—}670, orf4_{—}670, orf5_{—}670, orf6_{—}670, orf7_{—}670, orf8_{—}670.

A preferred immunogenic composition of the invention comprises a S. pneumoniae strain 670 AI surface protein which may be formulated or purified in an oligomeric (pilus) form. Another preferred immunogenic composition of the invention comprises a S. pneumoniae strain 670 AI surface protein which has been isolated in an oligomeric (pilus) form.

One or more of the S. pneumoniae strain 670 AI open reading frame polynucleotide sequences may be replaced by a polynucleotide sequence coding for a fragment of the replaced ORF. Alternatively, one or more of the S. pneumoniae strain 670 AI open reading frames may be replaced by a sequence having sequence homology to the replaced ORF.

One or more of the S. pneumoniae strain 670 AI surface protein sequences typically include an LPXTG motif (e.g., SEQ ID NO:122) or other sortase substrate motif.

The S. pneumoniae strain 670 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae strain 670 AI surface proteins are capable of binding to or otherwise associating with an epithelial cell surface. S. pneumoniae strain 670 AI surface proteins may also be able to bind to or associate with fibrinogen, fibronectin, or collagen.

The S. pneumoniae strain 670 AI sortase proteins are predicted to be involved in the secretion and anchoring of the LPXTG containing surface proteins. S. pneumoniae strain 670 AI may encode for at least one surface protein. Alternatively, S. pneumoniae strain 670 AI may encode for at least two surface exposed proteins and at least one sortase. Preferably, S. pneumoniae strain 670 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae strain 670 AI surface protein such as orf3_{—}670, orf4_{—}670, or orf5_{—}670. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae strain 670 AI protein in oligomeric form, preferably in a hyperoligomeric form. In one embodiment, the invention comprises a composition comprising one or more S. pneumoniae strain 670 AI proteins and one or more S. pneumoniae from TIGR4 AI proteins, wherein one or more of the S. pneumoniae AI proteins is in the form of an oligomer, preferably in a hyperoligomeric form.

In addition to the open reading frames encoding the S. pneumoniae strain 670 AI proteins, S. pneumoniae strain 670 AI may also include a transcriptional regulator.

S. pneumoniae Strain 14 CSR 10 Adhesin Island

As discussed above, Applicants have identified adhesin islands within the genome of S. pneumoniae strain 14 CSR 10. The S. pneumoniae strain 14 CSR 10 Adhesin Island comprises a series of approximately seven open reading frames encoding for a collection of amino acid sequences comprising surface proteins and sortases. Specifically, the S. pneumoniae strain 14 CSR 10 AI proteins includes open reading frames encoding for two or more (i.e., 2, 3, 4, 5, 6, or 7) of ORF2_{—}14CSR, ORF3_{—}14CSR, ORF4_{—}14CSR, ORF5_{—}14CSR, ORF6_{—}14CSR, ORF7_{—}14CSR, ORF8_{—}14CSR.

A preferred immunogenic composition of the invention comprises a S. pneumoniae strain 14 CSR 10 AI surface protein which may be formulated or purified in an oligomeric (pilus) form. Another preferred immunogenic composition of the invention comprises a S. pneumoniae strain 14 CSR 10 AI surface protein which has been isolated in an oligomeric (pilus) form.

One or more of the S. pneumoniae strain 14 CSR 10 AI open reading frame polynucleotide sequences may be replaced by a polynucleotide sequence coding for a fragment of the replaced ORF. Alternatively, one or more of the S. pneumoniae strain 14 CSR 10 AI open reading frames may be replaced by a sequence having sequence homology to the replaced ORF.

One or more of the S. pneumoniae strain 14 CSR 10 AI surface protein sequences typically include an LPXTG motif (e.g., SEQ ID NO:122) or other sortase substrate motif.

The S. pneumoniae strain 14 CSR 10 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae strain 14 CSR 10 AI surface proteins are capable of binding to or otherwise associating with an epithelial cell surface. S. pneumoniae strain 14 CSR 10 AI surface proteins may also be able to bind to or associate with fibrinogen, fibronectin, or collagen.

The S. pneumoniae strain 14 CSR 10 AI sortase proteins are predicted to be involved in the secretion and anchoring of the LPXTG containing surface proteins. S. pneumoniae strain 14 CSR 10 AI may encode for at least one surface protein. Alternatively, S. pneumoniae strain 14 CSR 10 AI may encode for at least two surface exposed proteins and at least one sortase. Preferably, S. pneumoniae strain 14 CSR 10 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae strain 14 CSR 10 AI surface protein such as orf3_CSR, orf4_CSR, or orf5_CSR. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae strain 14 CSR 10 AI protein in oligomeric form, preferably in a hyperoligomeric form. In one embodiment, the invention comprises a composition comprising one or more S. pneumoniae strain 14 CSR 10 AI proteins, and one or more AI proteins of any of S. pneumoniae from TIGR4, 670, 19A Hungary 6, 6B Finland 12, 6B Spain 2, 9V Spain 3, 19F Taiwan 14, 23F Taiwan 15, or 23F Poland 16, wherein one or more of the S. pneumoniae AI proteins is in the form of an oligomer, preferably in a hyperoligomeric form.

In addition to the open reading frames encoding the S. pneumoniae strain 14 CSR 10AI proteins, S. pneumoniae strain 14 CSR 10 AI may also include a transcriptional regulator.

S. pneumoniae Strain 19A Hungary 6 Adhesin Island

As discussed above, Applicants have identified adhesin islands within the genome of S. pneumoniae strain 19A Hungary 6. The S. pneumoniae strain 19A Hungary 6 Adhesin Island comprises a series of approximately seven open reading frames encoding for a collection of amino acid sequences comprising surface proteins and sortases. Specifically, the S. pneumoniae strain 19A Hungary 6 AI proteins includes open reading frames encoding for two or more (i.e., 2, 3, 4, 5, 6, or 7) of ORF2_{—}19AH, ORF3_{—}19AH, ORF4_{—}19AH, ORF5_{—}19AH, ORF6_{—}19AH, ORF7_{—}19AH, ORF8_{—}19AH.

A preferred immunogenic composition of the invention comprises a S. pneumoniae strain 19A Hungary 6 AI surface protein which may be formulated or purified in an oligomeric (pilus) form. Another preferred immunogenic composition of the invention comprises a S. pneumoniae strain 19A Hungary 6 AI surface protein which has been isolated in an oligomeric (pilus) form.

One or more of the S. pneumoniae strain 19A Hungary 6 AI open reading frame polynucleotide sequences may be replaced by a polynucleotide sequence coding for a fragment of the replaced ORF. Alternatively, one or more of the S. pneumoniae strain 19A Hungary 6 AI open reading frames may be replaced by a sequence having sequence homology to the replaced ORF.

One or more of the S. pneumoniae strain 19A Hungary 6 AI surface protein sequences typically include an LPXTG motif (e.g., SEQ ID NO:122) or other sortase substrate motif.

The S. pneumoniae strain 19A Hungary 6 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae strain 19A Hungary 6 AI surface proteins are capable of binding to or otherwise associating with an epithelial cell surface. S. pneumoniae strain 19A Hungary 6 AI surface proteins may also be able to bind to or associate with fibrinogen, fibronectin, or collagen.

The S. pneumoniae strain 19A Hungary 6 AI sortase proteins are predicted to be involved in the secretion and anchoring of the LPXTG containing surface proteins. S. pneumoniae strain 19A Hungary 6 AI may encode for at least one surface protein. Alternatively, S. pneumoniae strain 19A Hungary 6 AI may encode for at least two surface exposed proteins and at least one sortase. Preferably, S. pneumoniae strain 19A Hungary 6 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae strain 19A Hungary 6 AI surface protein such as orf3_{—}19AH, orf4_{—}19AH, or orf5_{—}19AH. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae strain 19A Hungary 6 AI protein in oligomeric form, preferably in a hyperoligomeric form. In one embodiment, the invention comprises a composition comprising one or more S. pneumoniae strain 19A Hungary 6 AI proteins and one or more AI proteins from one of any one of S. pneumoniae from TIGR4, 670, 14 CSR 10, 6B Finland 12, 6B Spain 2, 9V Spain 3, 19F Taiwan 14, 23F Taiwan 15, or 23F Poland 16 AI GR4 AI proteins, wherein one or more of the S. pneumoniae AI proteins is in the form of an oligomer, preferably in a hyperoligomeric form.

In addition to the open reading frames encoding the S. pneumoniae strain 19A Hungary 6 AI proteins, S. pneumoniae strain 19A Hungary 6 AI may also include a transcriptional regulator.

S. pneumoniae Strain 19F Taiwan 14 Adhesin Island

As discussed above, Applicants have identified adhesin islands within the genome of S. pneumoniae strain 19F Taiwan 14. The S. pneumoniae strain 19F Taiwan 14 Adhesin Island comprises a series of approximately seven open reading frames encoding for a collection of amino acid sequences comprising surface proteins and sortases. Specifically, the S. pneumoniae strain 19F Taiwan 14 AI proteins includes open reading frames encoding for two or more (i.e., 2, 3, 4, 5, 6, or 7) of ORF2_{—}19FTW, ORF3_{—}19FTW, ORF4_{—}19FTW, ORF5_{—}19FTW, ORF6_{—}19FTW, ORF7_{—}19FTW, ORF8_{—}19FTW.

A preferred immunogenic composition of the invention comprises a S. pneumoniae strain 19F Taiwan 14 AI surface protein which may be formulated or purified in an oligomeric (pilus) form. Another preferred immunogenic composition of the invention comprises a S. pneumoniae strain 19F Taiwan 14 AI surface protein which has been isolated in an oligomeric (pilus) form.

One or more of the S. pneumoniae strain 19F Taiwan 14 AI open reading frame polynucleotide sequences may be replaced by a polynucleotide sequence coding for a fragment of the replaced ORF. Alternatively, one or more of the S. pneumoniae strain 19F Taiwan 14 AI open reading frames may be replaced by a sequence having sequence homology to the replaced ORF.

One or more of the S. pneumoniae strain 19F Taiwan 14 AI surface protein sequences typically include an LPXTG motif (e.g., SEQ ID NO:122) or other sortase substrate motif.

The S. pneumoniae strain 19F Taiwan 14 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae strain 19F Taiwan 14 AI surface proteins are capable of binding to or otherwise associating with an epithelial cell surface. S. pneumoniae strain 19F Taiwan 14 AI surface proteins may also be able to bind to or associate with fibrinogen, fibronectin, or collagen.

The S. pneumoniae strain 19F Taiwan 14 AI sortase proteins are predicted to be involved in the secretion and anchoring of the LPXTG containing surface proteins. S. pneumoniae strain 19F Taiwan 14 AI may encode for at least one surface protein. Alternatively, S. pneumoniae strain 19F Taiwan 14 AI may encode for at least two surface exposed proteins and at least one sortase. Preferably, S. pneumoniae strain 19F Taiwan 14 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae strain 19F Taiwan 14 AI surface protein such as orf3_{—}19FTW, orf4_{—}19FTW, or orf5_{—}19FTW. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae strain 19F Taiwan 14 AI protein in oligomeric form, preferably in a hyperoligomeric form. In one embodiment, the invention comprises a composition comprising one or more S. pneumoniae strain 19F Taiwan 14 AI proteins and one or more AI proteins of any one or more of S. pneumoniae from TIGR4, 670, 19A Hungary 6, 6B Finland 12, 6B Spain 2, 9V Spain 3, 14 CSR 10, 23F Taiwan 15, or 23F Poland 16, wherein one or more of the S. pneumoniae AI proteins is in the form of an oligomer, preferably in a hyperoligomeric form.

In addition to the open reading frames encoding the S. pneumoniae strain 19F Taiwan 14 AI proteins, S. pneumoniae strain 19F Taiwan 14 AI may also include a transcriptional regulator.

S. pneumoniae Strain 23F Poland 16 Adhesin Island

As discussed above, Applicants have identified adhesin islands within the genome of S. pneumoniae strain 23F Poland 16. The S. pneumoniae strain 23F Poland 16 Adhesin Island comprises a series of approximately seven open reading frames encoding for a collection of amino acid sequences comprising surface proteins and sortases. Specifically, the S. pneumoniae strain 23F Poland 16 AI proteins includes open reading frames encoding for two or more (i.e., 2, 3, 4, 5, 6, or 7) of ORF2_{—}23FP, ORF3_{—}23FP, ORF4_{—}23FP, ORF5_{—}23FP, ORF6_{—}23FP, ORF7_{—}23FP, and ORF8_{—}23FP.

A preferred immunogenic composition of the invention comprises a S. pneumoniae strain 23F Poland 16 AI surface protein which may be formulated or purified in an oligomeric (pilus) form. Another preferred immunogenic composition of the invention comprises a S. pneumoniae strain 23F Poland 16 AI surface protein which has been isolated in an oligomeric (pilus) form.

One or more of the S. pneumoniae strain 23F Poland 16 AI open reading frame polynucleotide sequences may be replaced by a polynucleotide sequence coding for a fragment of the replaced ORF. Alternatively, one or more of the S. pneumoniae strain 23F Poland 16 AI open reading frames may be replaced by a sequence having sequence homology to the replaced ORF.

One or more of the S. pneumoniae strain 23F Poland 16 AI surface protein sequences typically include an LPXTG motif (e.g., SEQ ID NO:122) or other sortase substrate motif.

The S. pneumoniae strain 23F Poland 16 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae strain 23F Poland 16 AI surface proteins are capable of binding to or otherwise associating with an epithelial cell surface. S. pneumoniae strain 23F Poland 16 AI surface proteins may also be able to bind to or associate with fibrinogen, fibronectin, or collagen.

The S. pneumoniae strain 23F Poland 16 AI sortase proteins are predicted to be involved in the secretion and anchoring of the LPXTG containing surface proteins. S. pneumoniae strain 23F Poland 16 AI may encode for at least one surface protein. Alternatively, S. pneumoniae strain 23F Poland 16 AI may encode for at least two surface exposed proteins and at least one sortase. Preferably, S. pneumoniae strain 23F Poland 16 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae strain 23F Poland 16 AI surface protein such as orf3_{—}23FP, orf4_{—}23FP, or orf5_{—}23FP. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae strain 23F Poland 16 AI protein in oligomeric form, preferably in a hyperoligomeric form. In one embodiment, the invention comprises a composition comprising one or more S. pneumoniae strain 23F Poland 16 AI proteins and one or more AI proteins from any one or more S. pneumoniae strains of TIGR4, 670, 19A Hungary 6, 6B Finland 12, 6B Spain 2, 9V Spain 3, 19F Taiwan 14, 23F Taiwan 15, or 14 CSR 10, wherein one or more of the S. pneumoniae AI proteins is in the form of an oligomer, preferably in a hyperoligomeric form.

In addition to the open reading frames encoding the S. pneumoniae strain 23F Poland 16 AI proteins, S. pneumoniae strain 23F Poland 16 AI may also include a transcriptional regulator.

S. pneumoniae Strain 23F Taiwan 15 Adhesin Island

As discussed above, Applicants have identified adhesin islands within the genome of S. pneumoniae strain 23F Taiwan 15. The S. pneumoniae strain 23F Taiwan 15 Adhesin Island comprises a series of approximately seven open reading frames encoding for a collection of amino acid sequences comprising surface proteins and sortases. Specifically, the S. pneumoniae strain 23F Taiwan 15 AI proteins includes open reading frames encoding for two or more (i.e., 2, 3, 4, 5, 6, or 7) of ORF2_{—}23FTW, ORF3_{—}23FTW, ORF4_{—}23FTW, ORF5_{—}23FTW, ORF6_{—}23FTW, ORF7_{—}23FTW, ORF8_{—}23FTW.

A preferred immunogenic composition of the invention comprises a S. pneumoniae strain 23F Taiwan 15 AI surface protein which may be formulated or purified in an oligomeric (pilus) form. Another preferred immunogenic composition of the invention comprises a S. pneumoniae strain 23F Taiwan 15 AI surface protein which has been isolated in an oligomeric (pilus) form.

One or more of the S. pneumoniae strain 23F Taiwan 15 AI open reading frame polynucleotide sequences may be replaced by a polynucleotide sequence coding for a fragment of the replaced ORF. Alternatively, one or more of the S. pneumoniae strain 23F Taiwan 15 AI open reading frames may be replaced by a sequence having sequence homology to the replaced ORF.

One or more of the S. pneumoniae strain 23F Taiwan 15 AI surface protein sequences typically include an LPXTG motif (e.g., SEQ ID NO:122) or other sortase substrate motif.

The S. pneumoniae strain 23F Taiwan 15 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae strain 23F Taiwan 15 AI surface proteins are capable of binding to or otherwise associating with an epithelial cell surface. S. pneumoniae strain 23F Taiwan 15 AI surface proteins may also be able to bind to or associate with fibrinogen, fibronectin, or collagen.

The S. pneumoniae strain 23F Taiwan 15 AI sortase proteins are predicted to be involved in the secretion and anchoring of the LPXTG containing surface proteins. S. pneumoniae strain 23F Taiwan 15 AI may encode for at least one surface protein. Alternatively, S. pneumoniae strain 23F Taiwan 15 AI may encode for at least two surface exposed proteins and at least one sortase. Preferably, S. pneumoniae strain 23F Taiwan 15 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae strain 23F Taiwan 15 AI surface protein such as orf3_{—}23FTW, orf4_{—}23FTW, or orf5_{—}23FTW. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae strain 23F Taiwan 15 AI protein in oligomeric form, preferably in a hyperoligomeric form. In one embodiment, the invention comprises a composition comprising one or more S. pneumoniae strain 23F Taiwan 15 AI proteins and one or more AI proteins from any one or more of S. pneumoniae from TIGR4, 670, 19A Hungary 6, 6B Finland 12, 6B Spain 2, 9V Spain 3, 19F Taiwan 14, 14 CSR 10, or 23F Poland 16 AI, wherein one or more of the S. pneumoniae AI proteins is in the form of an oligomer, preferably in a hyperoligomeric form.

In addition to the open reading frames encoding the S. pneumoniae strain 23F Taiwan 15 AI proteins, S. pneumoniae strain 23F Taiwan 15 AI may also include a transcriptional regulator.

S. pneumoniae Strain 6B Finland 12 Adhesin Island

As discussed above, Applicants have identified adhesin islands within the genome of S. pneumoniae strain 6B Finland 12. The S. pneumoniae strain 6B Finland 12 Adhesin Island comprises a series of approximately seven open reading frames encoding for a collection of amino acid sequences comprising surface proteins and sortases. Specifically, the S. pneumoniae strain 6B Finland 12 AI proteins includes open reading frames encoding for two or more (i.e., 2, 3, 4, 5, 6, or 7) of ORF2_{—}6BF, ORF3_{—}6BF, ORF4_{—}6BF, ORF56B_F, ORF6_{—}6BF, ORF7_{—}6BF, ORF8_{—}6BF.

A preferred immunogenic composition of the invention comprises a S. pneumoniae strain 6B Finland 12 AI surface protein which may be formulated or purified in an oligomeric (pilus) form. Another preferred immunogenic composition of the invention comprises a S. pneumoniae strain 6B Finland 12 AI surface protein which has been isolated in an oligomeric (pilus) form.

One or more of the S. pneumoniae strain 6B Finland 12 AI open reading frame polynucleotide sequences may be replaced by a polynucleotide sequence coding for a fragment of the replaced ORF. Alternatively, one or more of the S. pneumoniae strain 6B Finland 12 AI open reading frames may be replaced by a sequence having sequence homology to the replaced ORF.

One or more of the S. pneumoniae strain 6B Finland 12 AI surface protein sequences typically include an LPXTG motif (e.g., SEQ ID NO:122) or other sortase substrate motif.

The S. pneumoniae strain 6B Finland 12 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae strain 6B Finland 12 AI surface proteins are capable of binding to or otherwise associating with an epithelial cell surface. S. pneumoniae strain 6B Finland 12 AI surface proteins may also be able to bind to or associate with fibrinogen, fibronectin, or collagen.

The S. pneumoniae strain 6B Finland 12 AI sortase proteins are predicted to be involved in the secretion and anchoring of the LPXTG containing surface proteins. S. pneumoniae strain 6B Finland 12 AI may encode for at least one surface protein. Alternatively, S. pneumoniae strain 6B Finland 12 AI may encode for at least two surface exposed proteins and at least one sortase. Preferably, S. pneumoniae strain 6B Finland 12 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae strain 6B Finland 12 AI surface protein such as orf3_{—}6BF, orf4_{—}6BF, or orf5_{—}6BF. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae strain 6B Finland 12 AI protein in oligomeric form, preferably in a hyperoligomeric form. In one embodiment, the invention comprises a composition comprising one or more S. pneumoniae strain 6B Finland 12 AI proteins and one or more AI proteins of any one or more of S. pneumoniae from TIGR4, 670, 19A Hungary 6, 6B Finland 12, 6B Spain 2, 9V Spain 3, 19F Taiwan 14, 23F Taiwan 15, or 23F Poland 16 AI, wherein one or more of the S. pneumoniae AI proteins is in the form of an oligomer, preferably in a hyperoligomeric form.

In addition to the open reading frames encoding the S. pneumoniae strain 6B Finland 12 AI proteins, S. pneumoniae strain 6B Finland 12 AI may also include a transcriptional regulator.

S. pneumoniae Strain 6B Spain 2 Adhesin Island

As discussed above, Applicants have identified adhesin islands within the genome of S. pneumoniae strain 6B Spain 2. The S. pneumoniae strain 6B Spain 2 Adhesin Island comprises a series of approximately seven open reading frames encoding for a collection of amino acid sequences comprising surface proteins and sortases. Specifically, the S. pneumoniae strain 6B Spain 2 AI proteins includes open reading frames encoding for two or more (i.e., 2, 3, 4, 5, 6, or 7) of ORF2_{—}6BSP, ORF3_{—}6BSP, ORF4_{—}6BSP, ORF5_{—}6BSP, ORF6_{—}6BSP, ORF7_{—}6BSP, and ORF8_{—}6BSP.

A preferred immunogenic composition of the invention comprises a S. pneumoniae strain 6B Spain 2 AI surface protein which may be formulated or purified in an oligomeric (pilus) form. Another preferred immunogenic composition of the invention comprises a S. pneumoniae strain 6B Spain 2 AI surface protein which has been isolated in an oligomeric (pilus) form.

One or more of the S. pneumoniae strain 6B Spain 2 AI open reading frame polynucleotide sequences may be replaced by a polynucleotide sequence coding for a fragment of the replaced ORF. Alternatively, one or more of the S. pneumoniae strain 6B Spain 2 AI open reading frames may be replaced by a sequence having sequence homology to the replaced ORF.

One or more of the S. pneumoniae strain 6B Spain 2 AI surface protein sequences typically include an LPXTG motif (e.g., SEQ ID NO:122) or other sortase substrate motif.

The S. pneumoniae strain 6B Spain 2 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae strain 6B Spain 2 AI surface proteins are capable of binding to or otherwise associating with an epithelial cell surface. S. pneumoniae strain 6B Spain 2 AI surface proteins may also be able to bind to or associate with fibrinogen, fibronectin, or collagen.

The S. pneumoniae strain 6B Spain 2 AI sortase proteins are predicted to be involved in the secretion and anchoring of the LPXTG containing surface proteins. S. pneumoniae strain 6B Spain 2 AI may encode for at least one surface protein. Alternatively, S. pneumoniae strain 6B Spain 2 AI may encode for at least two surface exposed proteins and at least one sortase. Preferably, S. pneumoniae strain 6B Spain 2 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae strain 6B Spain 2 AI surface protein such as orf3_{—}6BSP, orf4_{—}6BSP, or orf5_{—}6BSP. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae strain 6B Spain 2 AI protein in oligomeric form, preferably in a hyperoligomeric form. In one embodiment, the invention comprises a composition comprising one or more S. pneumoniae strain 6B Spain 2 AI proteins and one or more AI proteins of any one or more of S. pneumoniae from TIGR4, 670, 19A Hungary 6, 6B Finland 12, 14 CSR 10, 9V Spain 3, 19F Taiwan 14, 23F Taiwan 15, or 23F Poland 16 AI, wherein one or more of the S. pneumoniae AI proteins is in the form of an oligomer, preferably in a hyperoligomeric form.

In addition to the open reading frames encoding the S. pneumoniae strain 6B Spain 2 AI proteins, S. pneumoniae strain 6B Spain 2 AI may also include a transcriptional regulator.

S. pneumoniae Strain 9V Spain 3 Adhesin Island

As discussed above, Applicants have identified adhesin islands within the genome of S. pneumoniae strain 9V Spain 3. The S. pneumoniae strain 9V Spain 3 Adhesin Island comprises a series of approximately seven open reading frames encoding for a collection of amino acid sequences comprising surface proteins and sortases. Specifically, the S. pneumoniae strain 9V Spain 3 AI proteins includes open reading frames encoding for two or more (i.e., 2, 3, 4, 5, 6, or 7) of ORF2_{—}9VSP, ORF3_{—}9VSP, ORF4_{—}9VSP, ORF5_{—}9VSP, ORF6_{—}9VSP, ORF7_{—}9VSP, and ORF8_{—}9VSP.

A preferred immunogenic composition of the invention comprises a S. pneumoniae strain 9V Spain 3 AI surface protein which may be formulated or purified in an oligomeric (pilus) form. Another preferred immunogenic composition of the invention comprises a S. pneumoniae strain 9V Spain 3 AI surface protein which has been isolated in an oligomeric (pilus) form.

One or more of the S. pneumoniae strain 9V Spain 3 AI open reading frame polynucleotide sequences may be replaced by a polynucleotide sequence coding for a fragment of the replaced ORF. Alternatively, one or more of the S. pneumoniae strain 9V Spain 3 AI open reading frames may be replaced by a sequence having sequence homology to the replaced ORF.

One or more of the S. pneumoniae strain 9V Spain 3 AI surface protein sequences typically include an LPXTG motif (e.g., SEQ ID NO:122) or other sortase substrate motif.

The S. pneumoniae strain 9V Spain 3 AI surface proteins of the invention may affect the ability of the S. pneumoniae bacteria to adhere to and invade epithelial cells. AI surface proteins may also affect the ability of S. pneumoniae to translocate through an epithelial cell layer. Preferably, one or more S. pneumoniae strain 9V Spain 3 AI surface proteins are capable of binding to or otherwise associating with an epithelial cell surface. S. pneumoniae strain 9V Spain 3 AI surface proteins may also be able to bind to or associate with fibrinogen, fibronectin, or collagen.

The S. pneumoniae strain 9V Spain 3 AI sortase proteins are predicted to be involved in the secretion and anchoring of the LPXTG containing surface proteins. S. pneumoniae strain 9V Spain 3 AI may encode for at least one surface protein. Alternatively, S. pneumoniae strain 9V Spain 3 AI may encode for at least two surface exposed proteins and at least one sortase. Preferably, S. pneumoniae strain 9V Spain 3 AI encodes for at least three surface exposed proteins and at least two sortases.

The AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722.

In one embodiment, the invention includes a composition comprising oligomeric, pilus-like structures comprising a S. pneumoniae strain 9V Spain 3 AI surface protein such as orf3_{—}9VSP, orf4_{—}9VSP, or orf5_{—}9VSP. The oligomeric, pilus-like structure may comprise numerous units of AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine or serine amino acid residue, respectively.

AI surface proteins or fragments thereof to be incorporated into the oligomeric, pilus-like structures of the invention will preferably include a pilin motif.

The oligomeric, pilus like structures may be used alone or in the combinations of the invention. In one embodiment, the invention comprises a S. pneumoniae strain 9V Spain 3 AI protein in oligomeric form, preferably in a hyperoligomeric form. In one embodiment, the invention comprises a composition comprising one or more S. pneumoniae strain 9V Spain 3 AI proteins and one or more AI proteins from any one or more of S. pneumoniae from TIGR4, 670, 19A Hungary 6, 6B Finland 12, 6B Spain 2, 14 CSR 10, 19F Taiwan 14, 23F Taiwan 15, or 23F Poland 16 AI, wherein one or more of the S. pneumoniae AI proteins is in the form of an oligomer, preferably in a hyperoligomeric form.

In addition to the open reading frames encoding the S. pneumoniae strain 9V Spain 3 AI proteins, S. pneumoniae strain 9V Spain 3 AI may also include a transcriptional regulator.

The S. pneumoniae oligomeric, pilus-like structures may be isolated or purified from bacterial cultures in which the bacteria express an S. pneumoniae AI surface protein. The invention therefore includes a method for manufacturing an oligomeric AI surface antigen comprising culturing a S. pneumoniae bacterium that expresses the oligomeric AI protein and isolating the expressed oligomeric AI protein from the S. pneumoniae bacteria. The AI protein may be collected from secretions into the supernatant or it may be purified from the bacterial surface. The method may further comprise purification of the expressed AI protein. Preferably, the AI protein is in a hyperoligomeric form.

The oligomeric, pilus-like structures may be isolated or purified from bacterial cultures overexpressing an AI surface protein. The invention therefore includes a method for manufacturing an S. pneumoniae oligomeric Adhesin Island surface antigen comprising culturing a S. pneumoniae bacterium adapted for increased AI protein expression and isolation of the expressed oligomeric Adhesin Island protein from the S. pneumoniae bacteria. The AI protein may be collected from secretions into the supernatant or it may be purified from the bacterial surface. The method may further comprise purification of the expressed Adhesin Island protein. Preferably, the Adhesin Island protein is in a hyperoligomeric form.

The S. pneumoniae bacteria are preferably adapted to increase AI protein expression by at least two (e.g., 2, 3, 4, 5, 8, 10, 15, 20, 25, 30, 35, 40, 45, 50, 60, 70, 80, 90, 100, 125, 150 or 200) times wild type expression levels.

S. pneumoniae bacteria may be adapted to increase AI protein expression by any means known in the art, including methods of increasing gene dosage and methods of gene upregulation. Such means include, for example, transformation of the S. pneumoniae bacteria with a plasmid encoding the AI protein. The plasmid may include a strong promoter or it may include multiple copies of the sequence encoding the AI protein. Optionally, the sequence encoding the AI protein within the S. pneumoniae bacterial genome may be deleted. Alternatively, or in addition, the promoter regulating the S. pneumoniae Adhesin Island may be modified to increase expression.

The invention further includes S. pneumoniae bacteria which have been adapted to produce increased levels of AI surface protein. In particular, the invention includes S. pneumoniae bacteria which have been adapted to produce oligomeric or hyperoligomeric AI surface protein. In one embodiment, the S. pneumoniae of the invention are inactivated or attenuated to permit in vivo delivery of the whole bacteria, with the AI surface protein exposed on its surface.

The invention further includes S. pneumoniae bacteria which have been adapted to have increased levels of expressed AI protein incorporated in pili on their surface. The S. pneumoniae bacteria may be adapted to have increased exposure of oligomeric or hyperoligomeric AI proteins on its surface by increasing expression levels of a signal peptidase polypeptide. Increased levels of a local signal peptidase expression in Gram positive bacteria (such us LepA in GAS) are expected to result in increased exposure of pili proteins on the surface of Gram positive bacteria. Increased expression of a leader peptidase in S. pneumoniae may be achieved by any means known in the art, such as increasing gene dosage and methods of gene upregulation. The S. pneumoniae bacteria adapted to have increased levels of leader peptidase may additionally be adapted to express increased levels of at least one pili protein.

Alternatively, the AI proteins of the invention may be expressed on the surface of a non-pathogenic Gram positive bacteria, such as Streptococcus gordonii (See, e.g., Byrd et al., “Biological consequences of antigen and cytokine co-expression by recombinant Streptococcus gordonii vaccine vectors, ” Vaccine (2002) 20:2197-2205) or Lactococcus lactis (See, e.g., Mannam et al., “Mucosal Vaccine Made from Live, Recombinant Lactococcus lactis Protects Mice against Pharyngeal Infection with Streptococcus pyogenes” Infection and Immunity (2004) 72(6):3444-3450). As used herein, non-pathogenic Gram positive bacteria refer to Gram positive bacteria which are compatible with a human host subject and are not associated with human pathogenesis. Preferably, the non-pathogenic bacteria are modified to express the AI surface protein in oligomeric, or hyper-oligomeric form. Sequences encoding for an AI surface protein and, optionally, an AI sortase, may be integrated into the non-pathogenic Gram positive bacterial genome or inserted into a plasmid. The non-pathogenic Gram positive bacteria may be inactivated or attenuated to facilitate in vivo delivery of the whole bacteria, with the AI surface protein exposed on its surface. Alternatively, the AI surface protein may be isolated or purified from a bacterial culture of the non-pathogenic Gram positive bacteria. For example, the AI surface protein may be isolated from cell extracts or culture supernatants. Alternatively, the AI surface protein may be isolated or purified from the surface of the non-pathogenic Gram positive bacteria.

The non-pathogenic Gram positive bacteria may be used to express any of the S. pneumoniae Adhesin Island proteins described herein. The non-pathogenic Gram positive bacteria are transformed to express an Adhesin Island surface protein. Preferably, the non-pathogenic Gram positive bacteria also express at least one Adhesin Island sortase. The AI transformed non-pathogenic Gram positive bacteria of the invention may be used to prevent or treat infection with pathogenic S. pneumoniae.

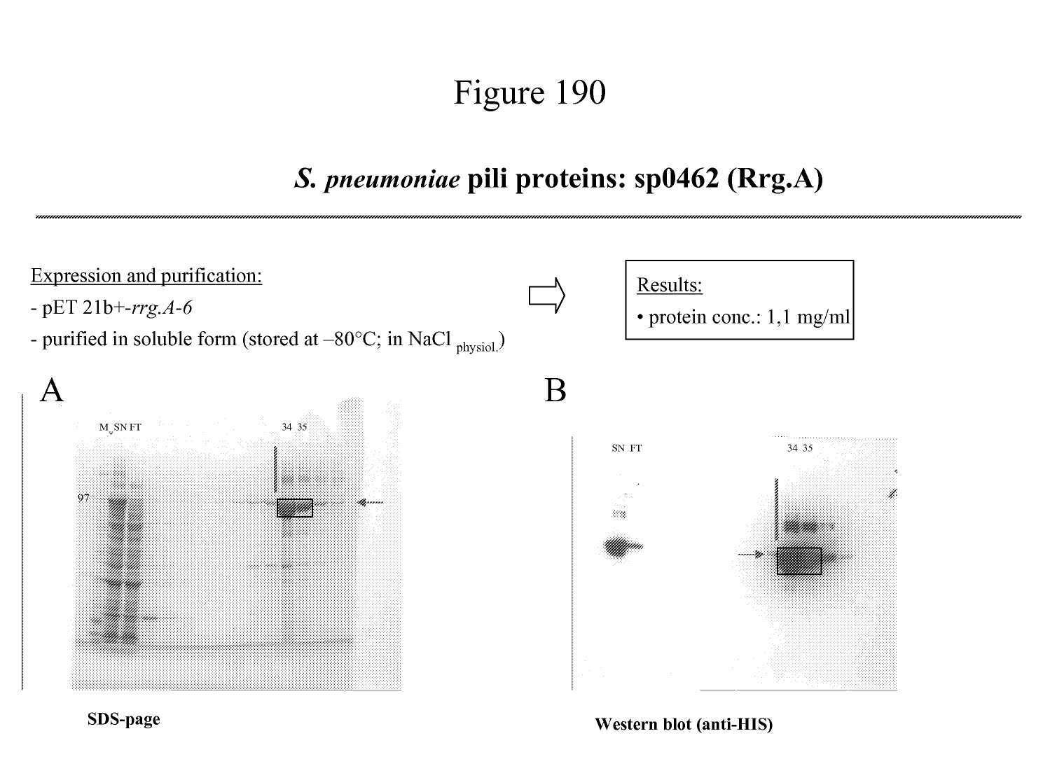

FIGS. 190A and B, and 193-195 provide examples of three methods successfully practiced by applicants to purify pili from S. pneumoniae TIGR4.

### Immunogenic Compositions

The Gram positive bacteria AI proteins described herein are useful in immunogenic compositions for the prevention or treatment of Gram positive bacterial infection. For example, the GBS AI surface proteins described herein are useful in immunogenic compositions for the prevention or treatment of GBS infection. As another example, the GAS AI surface proteins described herein may be useful in immunogenic compositions for the prevention or treatment of GAS infection. As another example, the S. pneumoniae AI surface proteins may be useful in immunogenic compositions for the prevention or treatment of S. pneumoniae infection.

Gram positive bacteria AI surface proteins that can provide protection across more than one serotype or strain isolate may be used to increase immunogenic effectiveness. For example, a particular GBS AI surface protein having an amino acid sequence that is at least 50% (i.e., at least 55%, 60%, 65%, 70%, 75%, 80%, 85%, 90%, 95%, 96%, 97%, 98%, 99%, or 100%) homologous to the particular GBS AI surface protein of at least 2 (i.e., at least 3, 4, 5, 6, 7, 8, 9, 10, or more) other GBS serotypes or strain isolates may be used to increase the effectiveness of such compositions.

As another example, fragments of Gram positive bacteria AI surface proteins that can provide protection across more than one serotype or strain isolate may be used to increase immunogenic effectiveness. Such a fragment may be identified within a consensus sequence of a full length amino acid sequence of a Gram positive bacteria AI surface protein. Such a fragment can be identified in the consensus sequence by its high degree of homology or identity across multiple (i.e, at least 3, 4, 5, 6, 7, 8, 9, 10, or more) Gram positive bacteria serotypes or strain isolates. Preferably, a high degree of homology is a degree of homology of at least 90% (i.e., at least 90%, 91%, 92%, 93%, 94%, 95%, 96%, 97%, 98%, 99%, or 100%) (across Gram positive bacteria serotypes or strain isolates. Preferably, a high degree of identity is a degree of identity of at least 90% (i.e., at least 90%, 91%, 92%, 93%, 94%, 95%, 96%, 97%, 98%, 99%, or 100%) across Gram positive bacteria serotypes or strain isolates. In one embodiment of the invention, such a fragment of a Gram positive bacteria AI surface protein may be used in the immunogenic compositions.

In addition, the AI surface protein oligomeric pilus structures may be formulated or purified for use in immunization. Isolated AI surface protein oligomeric pilus structures may also be used for immunization.

The invention includes an immunogenic composition comprising a first Gram positive bacteria AI protein and a second Gram positive bacterial AI protein. One or more of the AI proteins may be a surface protein. Such surface proteins may contain an LPXTG motif or other sortase substrate motif.

The first and second AI proteins may be from the same or different genus or species of Gram positive bacteria. If within the same species, the first and second AI proteins may be from the same or different AI subtypes. If two AIs are of the same subtype, the AIs have the same numerical designation. For example, all AIs designated as AI-1 are of the same AI subtype. If two AIs are of a different subtype, the AIs have different numerical designations. For example, AI-1 is of a different AI subtype from AI-2, AI-3, AI-4, etc. Likewise, AI-2 is of a different AI subtype from AI-1, AI-3, and AI-4, etc.

For example, the invention includes an immunogenic composition comprising one or more GBS AI-1 proteins and one or more GBS AI-2 proteins. One or more of the AI proteins may be a surface protein. Such surface proteins may contain an LPXTG motif (e.g., SEQ ID NO:122) and may bind fibrinogen, fibronectin, or collagen. One or more of the AI proteins may be a sortase. The GBS AI-1 proteins may be selected from the group consisting of GBS 80, GBS 104, GBS 52, SAG0647 and SAG0648. Preferably, the GBS AI-1 proteins include GBS 80 or GBS 104.

The GBS AI-2 proteins may be selected from the group consisting of GBS 67, GBS 59, GBS 150, SAG1405, SAG1406, 01520, 01521, 01522, 01523, 01523, 01524 and 01525. In one embodiment, the GBS AI-2 proteins are selected from the group consisting of GBS 67, GBS 59, GBS 150, SAG1405, and SAG1406. In another embodiment, the GBS AI-2 proteins may be selected from the group consisting of 01520, 01521, 01522, 01523, 01523, 01524 and 01525. Preferably, the GBS AI-2 protein includes GBS 59 or GBS 67.

As another example, the invention includes an immunogenic composition comprising one or more of any combination of GAS AI-1, GAS AI-2, GAS AI-3, or GAS AI-4 proteins. One or more of the GAS AI proteins may be a sortase. The GAS AI-1 proteins may be selected from the group consisting of M6_Spy0156, M6_Spy0157, M6_Spy0158, M6_Spy0159, M6_Spy0160, M6_Spy0161, DCD SS 410_fimbrial, ISS3650_fimbrial, and DSM2071_fimbrial. Preferably, the GAS AI-1 proteins are selected from the group consisting of M6_Spy0157, M6_Spy0159, M6_Spy0160, CDC SS 410_fimbrial, ISS3650_fimbrial, and DSM2071_fimbrial.

The GAS AI-2 proteins may be selected from the group consisting of Spy0124, GAS15, Spy0127, GAS16, GAS17, GAS18, Spy0131, Spy0133, and GAS20. Preferably, the GAS AI-2 proteins are selected from the group consisting of GAS 15, GAS16, and GAS18.

The GAS AI-3 proteins may be selected from the group consisting of SpyM3_{—}0097, SpyM3_{—}0098, SpyM3_{—}0099, SpyM3_{—}0100, SpyM3_{—}0101, SpyM3_{—}0102, SpyM3_{—}0103, SpyM3_{—}0104, SPs0099, SPs0100, SPs0101, SPs0102, SPs0103, SPs0104, SPs0105, SPs0106, orf77, orf78, orf79, orf80, orf81, orf82, orf83, orf84, spyM18_{—}0125, spyM18_{—}0126, spyM18_{—}0127, spyM18_{—}0128, spyM18_{—}0129, spyM18_{—}0130, spyM18_{—}0131, spyM18_{—}0132, SpyoM01000156, SpyoM01000155, SpyoM01000154, SpyoM01000153, SpyoM01000152, SpyoM01000151, SpyoM01000150, SpyoM01000149, ISS3040_fimbrial, ISS3776_fimbrial, and ISS4959_fimbrial. In one embodiment the GAS AI-3 proteins are selected from the group consisting of SpyM3_{—}0097, SpyM3_{—}0098, SpyM3_{—}0099, SpyM3_{—}0100, SpyM3_{—}0101, SpyM3_{—}0102, SpyM3_{—}0103, and SpyM3_{—}0104. In another embodiment, the GAS AI-3 proteins are selected from the group consisting of SPs0099, SPs0100, SPs0101, SPs0102, SPs0103, SPs0104, SPs0105, and SPs0106. In yet another embodiment, the GAS AI-3 proteins are selected from the group consisting of orf77, orf78, orf79, orf80, orf81, orf82, orf83, and orf84. In a further embodiment, the GAS AI-3 proteins are selected from the group consisting of spyM18_{—}0125, spyM18_{—}0126, spyM18_{—}0127, spyM18_{—}0128, spyM8_{—}0129, spyM18_{—}0130, spyM18_{—}0131, and spyM18_{—}0132. In yet another embodiment the GAS AI-3 proteins are selected from the group consisting of SpyoM01000156, SpyoM01000155, SpyoM01000154, SpyoM01000153, SpyoM01000152, SpyoM01000151, SpyoM01000150, and SpyoM01000149.

The GAS AI-4 proteins may be selected from the group consisting of 19224133, 19224134, 19224135, 19224136, 19224137, 19224138, 19224139, 19224140, 19224141, 20010296_fimbrial, 20020069_fimbrial, CDC SS 635_fimbrial, ISS4883_fimbrial, and ISS4538_fimbrial. Preferably, the GAS-AI4 proteins are selected from the group consisting of 19224134, 19224135, 19224137, 19224139, 19224141, 20010296_fimbrial, 20020069_fimbrial, CDC SS 635_fimbrial, ISS4883_fimbrial, and ISS4538_fimbrial.

As yet another example, the invention includes an immunogenic composition comprising one or more of any combination of S. pneumoniae from TIGR4, S. pneumoniae strain 670, S. pneumoniae from 19A Hungary 6, S. pneumoniae from 6B Finland 12, S. pneumoniae from 6B Spain 2, S. pneumoniae from 9V Spain 3, S. pneumoniae from 14 CSR 10, S. pneumoniae from 19F Taiwan 14, S. pneumoniae from 23F Taiwan 15, or S. pneumoniae from 23F Poland 16 AI proteins. One or more of the AI proteins may be a surface protein. Such surface proteins may contain an LPXTG motif (e.g., SEQ ID NO:122) and may bind fibrinogen, fibronectin, or collagen. One or more of the AI proteins may be a sortase.

The S. pneumoniae from TIGR4 AI proteins may be selected from the group consisting of SP0462, SP0463, SP0464, SP0465, SP0466, SP0467, SP0468. Preferably, the S. pneumoniae from TIGR4 AI proteins include SP0462, SP0463, or SP0464.

The S. pneumoniae strain 670 AI proteins may be selected from the group consisting of Orf1_{—}670, Orf3_{—}670, Orf4_{—}670, Orf5_{—}670, Orf6_{—}670, Orf7_{—}670, and Orf8_{—}670. Preferably, the S. pneumoniae strain 670 AI proteins include Orf3_{—}670, Orf4_{—}670, or Orf5_{—}670.

The S. pneumoniae from 19A Hungary 6 AI proteins may be selected from the group consisting of ORF2_{—}19AH, ORF3_{—}19AH, ORF4_{—}19AH, ORF5_{—}19AH, ORF6_{—}19AH, ORF7_{—}19AH, or ORF8_{—}19AH.

The S. pneumoniae from 6B Finland 12 AI proteins may be selected from the group consisting of ORF2_{—}6BF, ORF3_{—}6BF, ORF4_{—}6BF, ORF5_{—}6BF, ORF6_{—}6BF, ORF7_{—}6BF, ORF8_{—}6BF.

The S. pneumoniae from 6B Spain 2 AI proteins may be selected from the group consisting of ORF2_{—}6BSP, ORF3_{—}6BSP, ORF4_{—}6BSP, ORF5_{—}6BSP, ORF6_{—}6BSP, ORF7_{—}6BSP, or ORF8_BSP.

The S. pneumoniae from 9V Spain 3 AI proteins may be selected from the group consisting of ORF2_{—}9VSP, ORF3_{—}9VSP, ORF4_{—}9VSP, ORF5_{—}9VSP, ORF6_{—}9VSP, ORF7_{—}8VSP, or ORF8_{—}9VSP.

The S. pneumoniae from 14 CSR 10 AI proteins may be selected from the group consisting of ORF2_{—}14CSR, ORF3_{—}14CSR, ORF4_{—}14CSR, ORF5_{—}14CSR, ORF6_{—}14CSR, ORF7_{—}14CSR, or ORF8_{—}14CSR.

The S. pneumoniae from 19F Taiwan 14 AI proteins may be selected from the group consisting of ORF2_{—}19FTW, ORF3_{—}19FTW, ORF4_{—}19FTW, ORF5_{—}19FTW, ORF6_{—}19FTW, ORF7_{—}19FTW, or ORF8_{—}19FTW.

The S. pneumoniae from 23F Taiwan 15 AI proteins may be selected from the group consisting of ORF2_{—}23FTW, ORF3_{—}23FTW, ORF4_{—}23FTW, ORF5_{—}23FTW, ORF6_{—}23FTW, ORF7_{—}23FTW, or ORF8_{—}23FTW.

The S. pneumoniae from 23F Poland 16 AI proteins may be selected from the group consisting of ORF2_{—}23FP, ORF3_{—}23FP, ORF4_{—}23FP, ORF5_{—}23FP, ORF6_{—}23FP, ORF7_{—}23FP, or ORF8_{—}23FP.

Preferably, the Gram positive bacteria AI proteins included in the immunogenic compositions of the invention can provide protection across more than one serotype or strain isolate. For example, the immunogenic composition may comprise a first AI protein, wherein the amino acid sequence of said AI protein is at least 90% (i.e., at least 90, 91, 92, 93, 94, 95, 96, 97, 98, 99 or 100%) homologous to the amino acid sequence of a second AI protein, and wherein said first AI protein and said second AI protein are derived from the genomes of different serotypes of a Gram positive bacteria. The first AI protein may also be homologous to the amino acid sequence of a third AI protein, such that the first AI protein, the second AI protein and the third AI protein are derived from the genomes of different serotypes of a Gram positive bacteria. The first AI protein may also be homologous to the amino acid sequence of a fourth AI protein, such that the first AI protein, the second AI protein and the third AI protein are derived from the genomes of different serotypes of a Gram positive bacteria.

For example, preferably, the GBS AI proteins included in the immunogenic compositions of the invention can provide protection across more than one GBS serotype or strain isolate. For example, the immunogenic composition may comprise a first GBS AI protein, wherein the amino acid sequence of said AI protein is at least 90% (i.e., at least 90, 91, 92, 93, 94, 95, 96, 97, 98, 99 or 100%) homologous to the amino acid sequence of a second GBS AI protein, and wherein said first AI protein and said second AI protein are derived from the genomes of different GBS serotypes. The first GBS AI protein may also be homologous to the amino acid sequence of a third GBS AI protein, such that the first AI protein, the second AI protein and the third AI protein are derived from the genomes of different GBS serotypes. The first AI protein may also be homologous to the amino acid sequence of a fourth GBS AI protein, such that the first AI protein, the second AI protein and the third AI protein are derived from the genomes of different GBS serotypes.

The first AI protein may be selected from an AI-1 protein or an AI-2 protein. For example, the first AI protein may be a GBS AI-1 surface protein such as GBS 80. The amino acid sequence of GBS 80 from GBS serotype V, strain isolate 2603 is greater than 90% homologous to the GBS 80 amino acid sequence from GBS serotype III, strain isolates NEM316 and COH1 and the GBS 80 amino acid sequence from GBS serotype la, strain isolate A909.

As another example, the first AI protein may be GBS 104. The amino acid sequence of GBS 104 from GBS serotype V, strain isolate 2603 is greater than 90% homologous to the GBS 104 amino acid sequence from GBS serotype III, strain isolates NEM316 and COH1, the GBS 104 amino acid sequence from GBS serotype la, strain isolate A909, and the GBS 104 amino acid sequence serotype II, strain isolate 18RS21.

Table 12 provides the amino acid sequence identity of GBS 80 and GBS 104 across GBS serotypes Ia, Ib, II, III, V, and VIII. The GBS strains in which genes encoding GBS 80 and GBS 104 were identified share, on average, 99.88 and 99.96 amino acid sequence identity, respectively. This high degree of amino acid identity indicates that an immunogenic composition comprising a first protein of GBS 80 or GBS 104 may provide protection across more than one GBS serotype or strain isolate. As another example, the first AI protein may be an AI-2 protein such as GBS 67. The amino acid sequence of GBS 67 from GBS serotype V, strain isolate 2603 is greater than 90% homologous to the GBS 67 amino acid sequence from GBS serotype III, strain isolate NEM316, the GBS 67 amino acid sequence from GBS serotype 1b, strain isolate H36B, and the GBS 67 amino acid sequence from GBS serotype II, strain isolate 17RS21.

As another example, the first AI protein may be an AI-2 protein such as spb1. The amino acid sequence of spb1 from GBS serotype III, strain isolate COH1 is greater than 90% homologous to the spb1 amino acid sequence from GBS serotype Ia, strain isolate A909.

As yet another example, the first AI protein may be an AI-2 protein such as GBS 59. The amino acid sequence of GBS 59 from GBS serotype II, strain isolate 18RS21 is 100% homologous to the GBS 59 amino acid sequence from GBS serotype V, strain isolate 2603. The amino acid sequence of GBS 59 from GBS serotype V, strain isolate CJB111 is 98% homologous to the GBS 59 amino acid sequence from GBS serotype III, strain isolate NEM316.

The compositions of the invention may also be designed to include Gram positive AI proteins from divergent serotypes or strain isolates, i.e., to include a first AI protein which is present in one collection of serotypes or strain isolates of a Gram positive bacteria and a second AI protein which is present in those serotypes or strain isolates not represented by the first AI protein.

For example, the invention may include an immunogenic composition comprising a first and second Gram positive bacteria AI protein, wherein a polynucleotide sequence encoding for the full length sequence of the first AI protein is not present in a similar Gram positive bacterial genome comprising a polynucleotide sequence encoding for the second AI protein.

The compositions of the invention may also be designed to include AI proteins from divergent GBS serotypes or strain isolates, i.e., to include a first AI protein which is present in one collection of GBS serotypes or strain isolates and a second AI protein which is present in those serotypes or strain isolates not represented by the first AI protein.

For example, the invention may include an immunogenic composition comprising a first and second GBS AI protein, wherein a polynucleotide sequence encoding for the full length sequence of the first GBS AI protein is not present in a genome comprising a polynucleotide sequence encoding for the second GBS AI protein. For example, the first AI protein could be GBS 80 (such as the GBS 80 sequence from GBS serotype V, strain isolate 2603). As previously discussed (and depicted in FIG. 2), the sequence for GBS 80 in GBS serotype II, strain isolate 18RS21 is disrupted. In this instance, the second AI protein could be GBS 104 or GBS 67 (sequences selected from the GBS serotype II, strain isolate 18RS21).

Further, the invention may include an immunogenic composition comprising a first and second GBS AI protein, wherein the first GBS AI protein has detectable surface exposure on a first GBS strain or serotype but not a second GBS strain or serotype and the second GBS AI protein has detectable surface exposure on a second GBS strain or serotype but not a first GBS strain or serotype. For example, the first AI protein could be GBS 80 and the second AI protein could be GBS 67. As seen in Table 15, there are some GBS serotypes and strains that have surface exposed GBS 80 but that do not have surface exposed GBS 67 and vice versa. An immunogenic composition comprising a GBS 80 and a GBS 67 protein may provide protection across a wider group of GBS strains and serotypes.

TABLE 12
Conservation of GBS 80 and GBS 104 amino acid sequences

| | | GBS 80 | | GBS 104 | |
|---|---|---|---|---|---|
| Serotype | Strains | cGH | % AA identity | cGH | % AA identity |
| Ia | 090 | + | 99.79 | + | 100.00 |
| | A909 | + | 100.00 | + | 100.00 |
| | 515 | − | | − | |
| | DK1 | − | | − | |
| | DK8 | − | | − | |
| | Davis | − | | − | |
| Ib | 7357b | + | 100.00 | + | |
| | H36B | − | | − | |
| II | 18RS21 | − | | + | 100.00 |
| | DK21 | − | | − | |
| III | NEM316 | + | 100.00 | + | 100.00 |
| | COH31 | + | 100.00 | + | |
| | D136 | + | 100.00 | + | |
| | M732 | + | 100.00 | + | 99.88 |
| | COH1 | + | 99.79 | + | 99.88 |
| | M781 | + | 99.79 | + | 99.88 |
| No type | CJB110 | + | 99.37 | + | 100.00 |
| | 1169NT | − | | − | |
| V | CJB111 | + | 100.00 | + | 100.00 |
| | 2603 | + | 100.00 | + | 100.00 |
| VIII | JM130013 | + | 99.79 | + | 100.00 |
| | SMU014 | + | 100.00 | + | |

| | | | | |
|---|---|---|---|---|
| total | 14/22 | 99.88 +/− 0.19 | 15/22 | 99.96 +/− 0.056 |

TABLE 15
Antigen surface exposure of GBS 80, GBS 322, GBS 104, and GBS 67

| GBS strains | Type | GBS 80 | GBS 322 | GBS 104 | GBS 67 |
|---|---|---|---|---|---|
| DK1* | Ia | 0 | nd | 237 | 478 |
| DK8* | | 0 | 213 | 151 | 475 |
| Davis* | | 0 | 86 | 271 | 430 |
| 515* | | 0 | 227 | 262 | 409 |
| 090 | | 0 | 0 | 0 | 0 |
| A909 | | 0 | 0 | 0 | 0 |
| 2986 | | 0 | 0 | 157 | 397 |
| 5551 | | 0 | 36 | 384 | 485 |
| 2177 | Ib | 477 | 323 | 328 | 66 |
| H36B* | | 0 | 105 | 518 | 444 |
| 7357b- | | 91 | 102 | 309 | 316 |
| 2129 | | 57 | 71 | 132 | 0 |
| 5518 | | 31 | nd | 60 | 28 |
| COH1 | III | 305 | 130 | 305 | 0 |
| D136C | | 16 | 460 | 226 | 406 |
| COH31 | | 0 | 479 | 71 | 273 |
| M732 | | 105 | 292 | 101 | 0 |
| M781 | | 65 | 224 | 136 | 0 |
| 1998 | | 95 | 288 | 205 | 350 |
| 5376 | | 165 | 76 | 156 | 0 |
| 5435 | | 93 | 88 | 100 | 0 |
| 18RS21 | II | 0 | 471 | 50 | 103 |
| DK21* | | 0 | 342 | 419 | 331 |
| 3050 | | 43 | 188 | 289 | 460 |
| 5401 | | 170 | 135 | 494 | 618 |
| 2141 | | 0 | 76 | 0 | 69 |
| CJB111 | V | 365 | 58 | 355 | 481 |
| 2603 | | 62 | 293 | 100 | 105 |
| 5364 | | 454 | 463 | 379 | 394 |
| 2110 | | 0 | 11 | 345 | 589 |
| 2274 | IV | 113 | 161 | 465 | 484 |
| 1999 | | 0 | 55 | 492 | 453 |
| 2210 | | 0 | 0 | 363 | 574 |
| 2928 | VII | 0 | 0 | 0 | 0 |
| SMU071 | VIII | 556 | 170 | 393 | 79 |
| JM9130013 | | 587 | 133 | 436 | 83 |
| 2189 | | 0 | 0 | 0 | 0 |
| 5408 | | 0 | 0 | 159 | 433 |
| CJB110 | NT | 71 | 587 | 169 | 245 |
| 1169* | | 0 | 213 | 371 | 443 |
| Δ Mean >100 | | 9/40 | 22/38 | 32/40 | 25/40 |
| | | 22% | 58% | 80% | 62% |

Alternatively, the invention may include an immunogenic composition comprising a first and second Gram positive bacteria AI protein, wherein the polynucleotide sequence encoding the sequence of the first AI protein is less than 90% (i.e., less than 90, 88, 86, 84, 82, 80, 78, 76, 74, 72, 70, 65, 60, 55, 50, 45, 40, 35 or 30 percent) homologous than the corresponding sequence in the genome of the second AI protein.

The invention may include an immunogenic composition comprising a first and second GBS AI protein, wherein the polynucleotide sequence encoding the sequence of the first GBS AI protein is less than 90% (i.e., less than 90, 88, 86, 84, 82, 80, 78, 76, 74, 72, 70, 65, 60, 55, 50, 45, 40, 35 or 30 percent) homologous than the corresponding sequence in the genome of the second GBS AI protein. For example, the first GBS AI protein could be GBS 67 (such as the GBS 67 sequence from GBS serotype 1b, strain isolate H36B). As shown in FIGS. 2 and 4, the GBS 67 sequence for this strain is less than 90% homologous (87%) to the corresponding GBS 67 sequence in GBS serotype V, strain isolate 2603. In this instance, the second GBS AI protein could then be the GBS 80 sequence from GBS serotype V, strain isolate 2603.

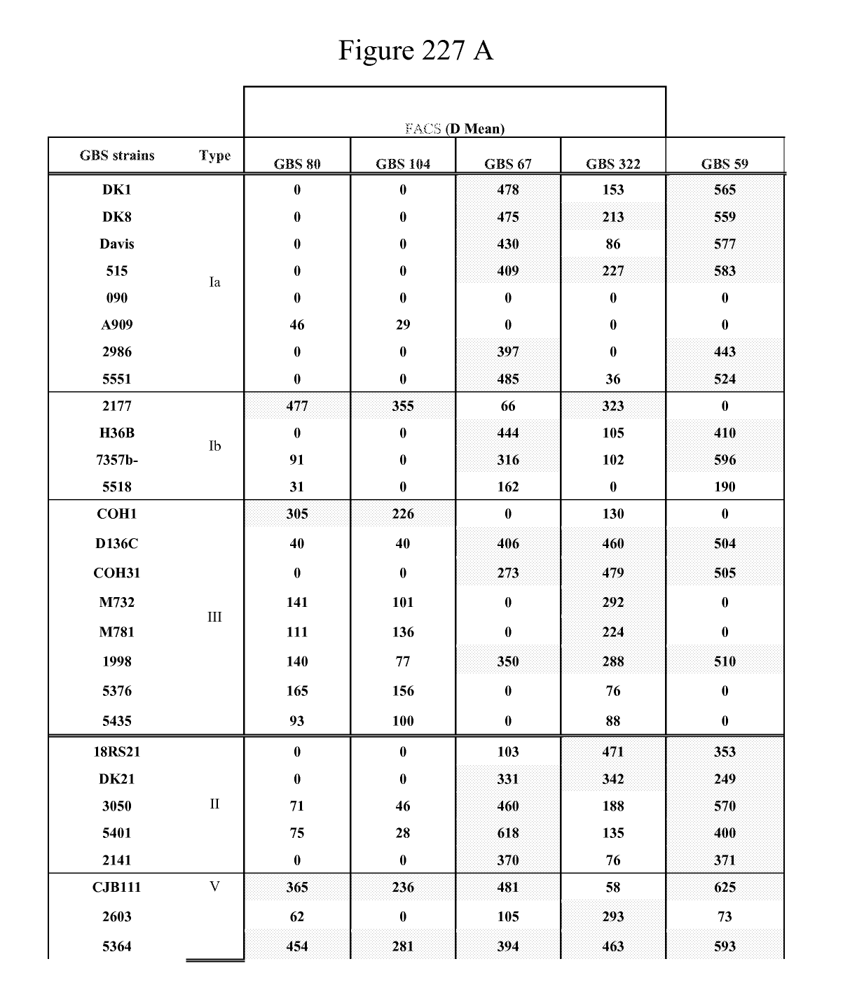

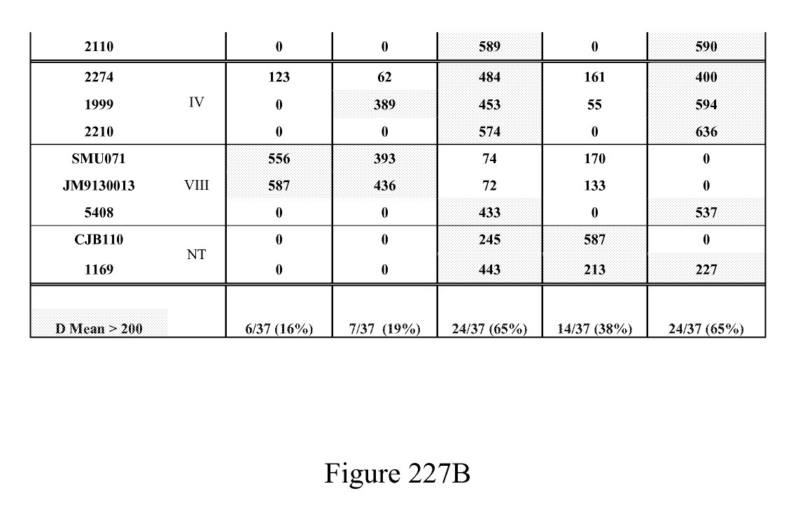

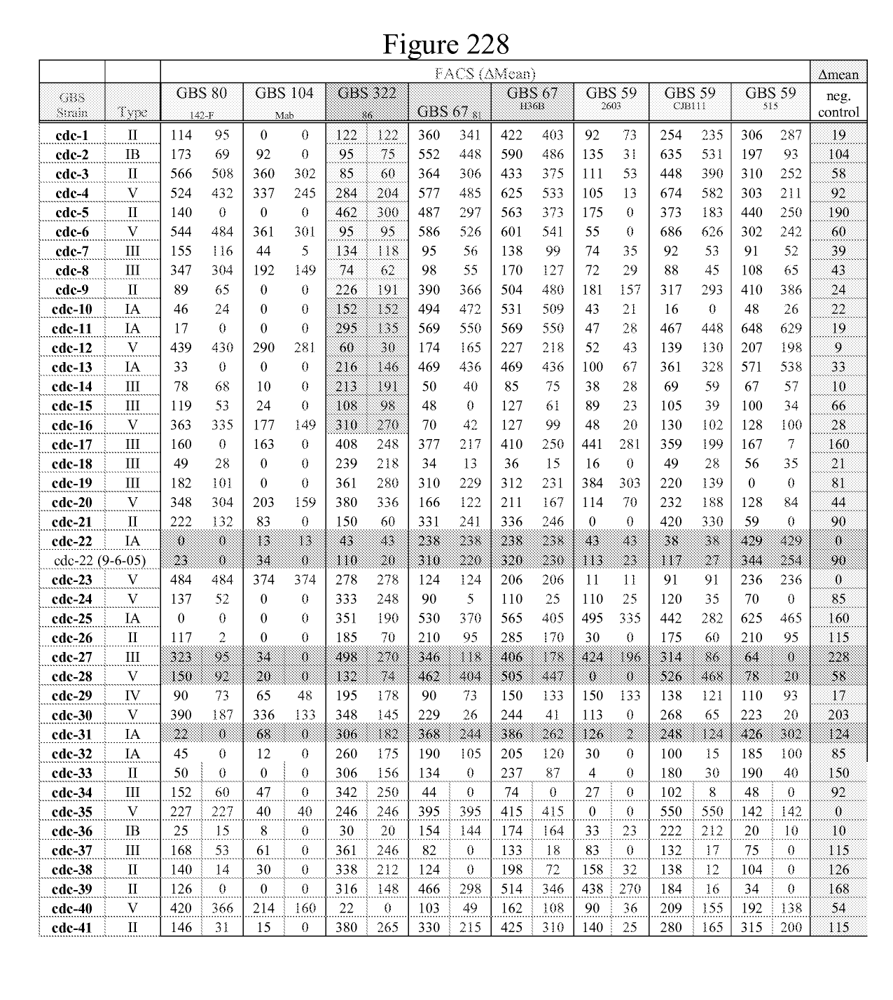

An example immunogenic composition of the invention may comprise adhesin island proteins GBS 80, GBS 104, GBS 67, and GBS 59, and non-AI protein GBS 322. FACS analysis of different GBS strains demonstrates that at least one of these five proteins is always found to be expressed on the surface of GBS bacteria. An initial FACS analysis of 70 strains of GBS bacteria, obtained from the CDC in the United States (33 strains), ISS in Italy (17 strains), and Houston/Harvard (20 strains), detected surface exposure of at least one of GBS 80, GBS 104, GBS 322, GBS 67, or GBS 59 on the surface of the GBS bacteria. FIG. 227 provides the FACS data obtained for surface exposure of GBS 80, GBS 104, GBS 67, GBS 322, and GBS 59 on each of 37 GBS strains. FIG. 228 provides the FACS data obtained for surface exposure of GBS 80, GBS 104, GBS 67, GBS 322, and GBS 59 on each of 41 GBS strains obtained from the CDC. As can be seen from FIGS. 227 and 228, each GBS strain had surface expression of at least one of GBS 80, GBS 104, GBS 67, GBS 322, and GBS 59. The surface exposure of at least one of these proteins on each bacterial strain indicates that an immunogenic composition comprising these proteins will provide wide protection across GBS strains and serotypes.

The surface exposed GBS 80, GBS 104, GBS 67, GBS 322, and GBS 59 proteins are also present at high levels as determined by FACS. Table 49 summarizes the FACS results for the initial 70 GBS strains examined for GBS 80, GBS 104, GBS 67, GBS 322, and GBS 59 surface expression. A protein was designated as having high levels of surface expression of a protein if a five-fold shift in fluorescence was observed when using antibodies for the protein relative to preimmune control serum.

TABLE 49
Exposure Levels of GBS 80, GBS 104, GBS 67, GBS 322,
and GBS 59 on GBS Strains

| | GBS 80 | GBS 104 | GBS 67 | GBS 59 | GBS 322 |
|---|---|---|---|---|---|
| 5-fold shift in | 17/70 | 14/70 | 49/70 | 46/70 | 33/70 |
| fluorescence | 24% | 20% | 70% | 66% | 47% |
| by FACS | | | | | |

Table 50 details which of the surface proteins is highly expressed on the different GBS serotype.

TABLE 50
High Levels of Surface Protein Expression on GBS Serotypes

| 5-fold shift in | | | | | |
|---|---|---|---|---|---|
| fluorescence | | | | | |
| by FACS | GBS 80 | GBS 104 | GBS 67 | GBS 59 | GBS 322 |
| Ia + Ib + III | 4/36 | 2/36 | 22/36 | 20/36 | 18/36 |
| II + V | 11/25 | 9/25 | 21/25 | 21/25 | 13/25 |
| Others | 2/9 | 3/9 | 6/9 | 5/9 | 2/9 |

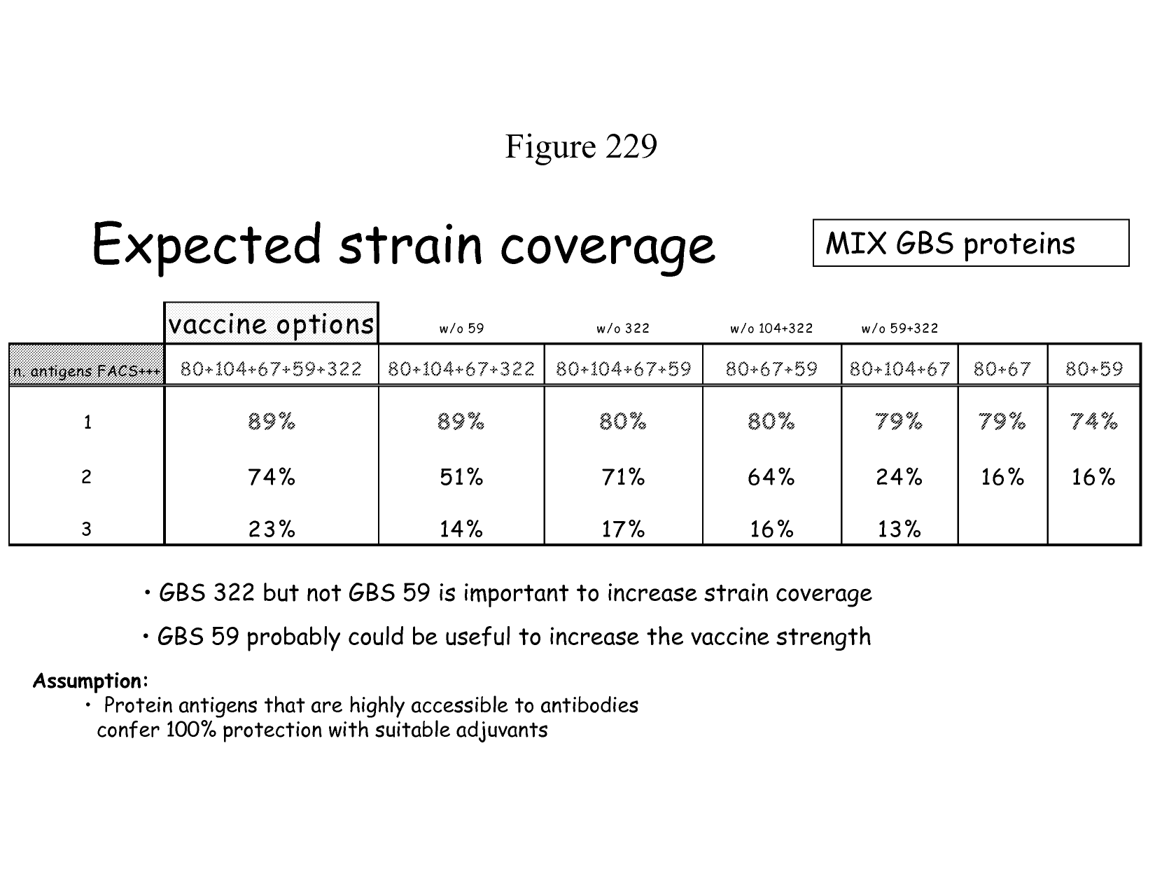

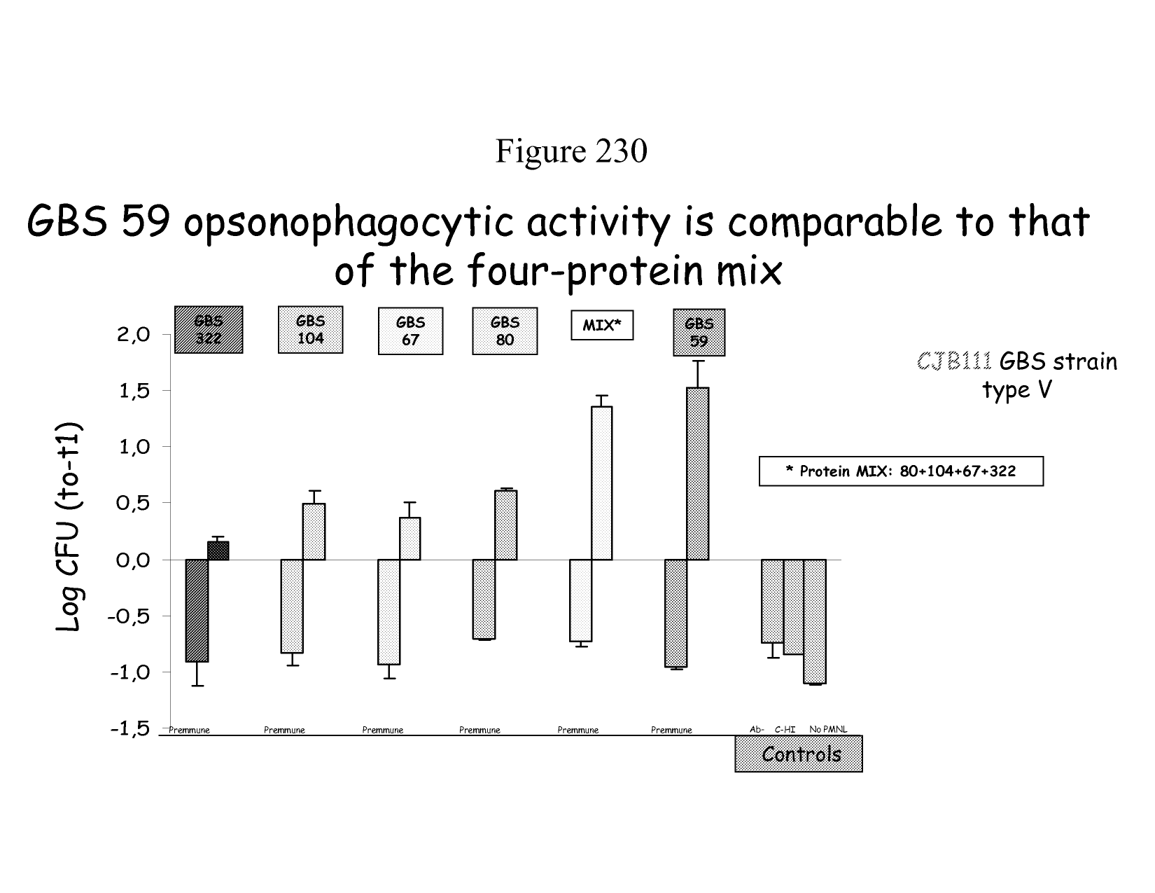

Alternatively, the immunogenic composition of the invention may include GBS 80, GBS 104, GBS 67, and GBS 322. Assuming that protein antigens that are highly accessible to antibodies confer 100% protection with suitable adjuvants, an immunogenic composition containing GBS 80, GBS 104, GBS 67, GBS 59 and GBS 322 will provide protection for 89% of GBS strains and serotypes, the same percentage as an immunogenic composition containing GBS 80, GBS 104, GBS 67, and GBS 322 proteins. See FIG. 229. However, it may be preferable to include GBS 59 in the composition to increase its immunogenic strength. As seen from Table 50, GBS 59 is highly expressed on the surface two-thirds of GBS bacteria examined by FACS analysis, unlike GBS 80, GBS 104, and GBS 322, which are highly expressed in less than half of GBS bacteria examined. GBS 59 opsonophagocytic activity is also comparable to that of a mix of GBS 322, GBS 104, GBS 67, and GBS 80 proteins. See FIG. 230.

The invention may include an immunogenic composition comprising a first and second GBS59 polypeptide, wherein the amino acidic sequence encoding the sequence of the first GBS59 polypeptide is less than 90% (i.e., less than 90, 88, 86, 84, 82, 80, 78, 76, 74, 72, 70, 65, 60, 55, 50, 45, 40, 35 or 30 percent) homologous than the corresponding sequence encoded in the genome of the second GBS59 polypeptide. As previously shown, 6 different allelic families of GBS59 polypeptides have been identified (see FIG. 240) which have less than 90% sequence identity. Accordingly, the first and the second GBS59 polypeptides of the invention include but are not limited to any one of the 6 allelic families ancestors (i.e., GBS59^{CJB111}, GBS59^{DK21}, GBS59⁵¹⁵, GBS59^{CJB110}, GBS59²⁶⁰³ and GBS59^{H36B}), leading to 15 possible combinations of two, For example, GBS59^{DK21} and GBS59^{CJB110}. Other possible such combinations are GBS59^{CJB111} and GBS56^{DK21}, GBS59^{CJB111} and GBS59⁵¹⁵, GBS59^{CJB111} and GBS59^{CJB110}, GBS59^{CJB111} and GBS59²⁶⁰³, GBS59^{CJB111} and GBS59^{H36B}, GBS59^{DK21} and GBS59⁵¹⁵, GBS59^{DK21} and GBS59^{2603,} GBS59^{DK21} and GBS59^{H36B}, GBS59⁵¹⁵ and GBS59^{CJB110}, GBS59⁵¹⁵ and GBS59^{2603,} GBS59⁵¹⁵ and GBS59^{H36B}, GBS59^{CJB110} and GBS59^{2603,} G59^{CJB110} and GBS59^{H36B} or GBS59²⁶⁰³ and GBS59^{H36B} GBS.

By way of another example, preferably, the GAS AI proteins included in the immunogenic compositions of the invention can provide protection across more than one GAS serotype or strain isolate. For example, the immunogenic composition may comprise a first GAS AI protein, wherein the amino acid sequence of said AI protein is at least 90% (i.e., at least 90, 91, 92, 93, 94, 95, 96, 97, 98, 99 or 100%) homologous to the amino acid sequence of a second GAS AI protein, and wherein said first AI protein and said second AI protein are derived from the genomes of different GAS serotypes. The first GAS AI protein may also be homologous to the amino acid sequence of a third GAS AI protein, such that the first AI protein, the second AI protein and the third AI protein are derived from the genomes of different GAS serotypes. The first AI protein may also be homologous to the amino acid sequence of a fourth GAS AI protein, such that the first AI protein, the second AI protein and the third AI protein are derived from the genomes of different GAS serotypes.

The compositions of the invention may also be designed to include GAS AI proteins from divergent serotypes or strain isolates, i.e., to include a first AI protein which is present in one collection of serotypes or strain isolates of a GAS bacteria and a second AI protein which is present in those serotypes or strain isolates not represented by the first AI protein.

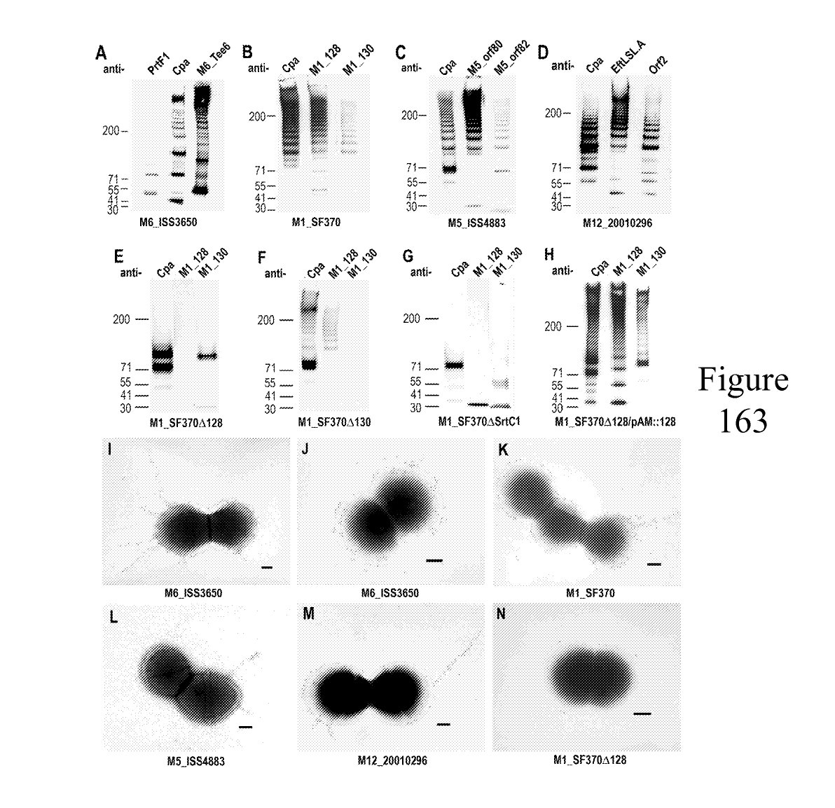

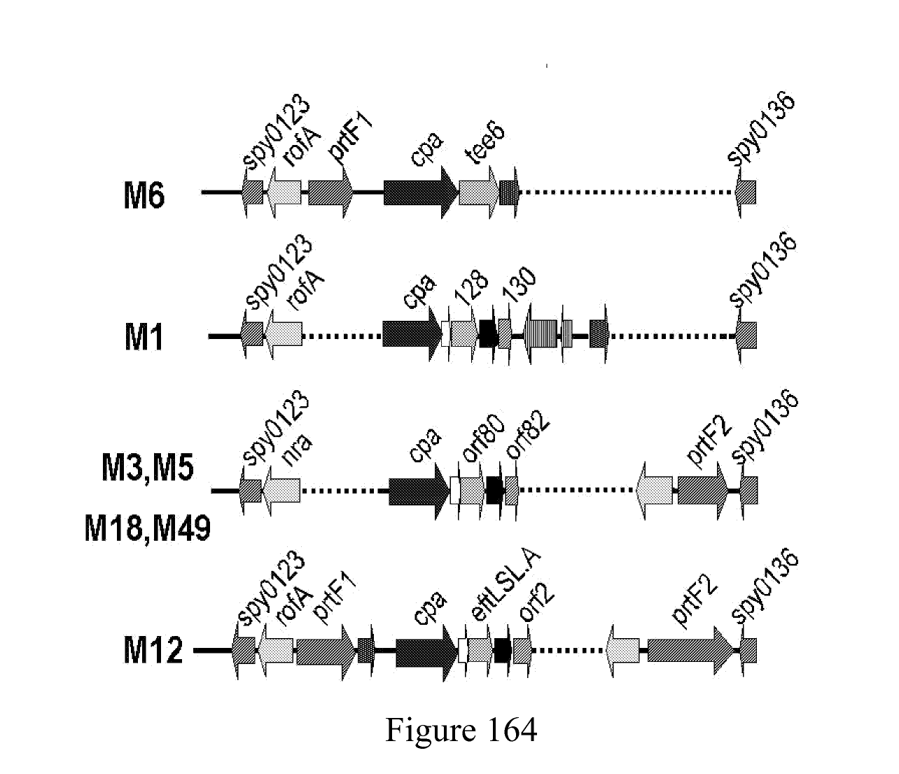

For example, the first AI protein could be a prtF2 protein (such as the 19224141 protein from GAS serotype M12, strain isolate A735). As previously discussed (and depicted in FIG. 164), the sequence for a prtF2 protein is not present in GAS AI types 1 or 2. In this instance, the second AI protein could be collagen binding protein M6_Spy0159 (from M6 isolate (MGAS10394), which comprises an AI-1) or GAS15 (from M1 isolate (SF370), which comprises an AI-2).

Further, the invention may include an immunogenic composition comprising a first and second GAS AI protein, wherein the first GAS AI protein has detectable surface exposure on a first GAS strain or serotype but not a second GAS strain or serotype and the second GAS AI protein has detectable surface exposure on a second GAS strain or serotype but not a first GAS strain or serotype.

The invention may include an immunogenic composition comprising a first and second GAS AI protein, wherein the polynucleotide sequence encoding the sequence of the first GAS AI protein is less than 90% (i.e., less than 90, 88, 86, 84, 82, 80, 78, 76, 74, 72, 70, 65, 60, 55, 50, 45, 40, 35 or 30 percent) homologous than the corresponding sequence in the genome of the second GAS AI protein. Preferably the first and second GAS AI proteins are subunits of the pilus. More preferably the first and second GAS AI proteins are selected from the major pilus forming proteins (i.e., M6_Spy0160 from M6 strain 10394, SPy0128 from M1 strain SF370, SpyM3_{—}0100 from M3 strain 315, SPs0102 from M3 strain SSI, orf80 from M5 isolate Manfredo, spyM18_{—}0128 from M18 strain 8232, SpyoM01000153 from M49 strain 591, 19224137 from M12 strain A735, fimbrial structural subunit from M77 strain ISS4959, fimbrial structural subunit from M44 strain ISS3776, fimbrial structural subunit from M50 strain ISS3776 ISS 4538, fimbrial structural subunit from M12strain CDC SS635, fimbrial structural subunit from M23 strain DSM2071, fimbrial structural subunit from M6 strain CDC SS410). Table 45 provides the percent identity between the amino acidic sequences of each of the main pilus forming subunits from GAS AI-1, AI-2, AI-3, and AI-4 representative strains (i.e., M6_Spy0160 from M6 strain 10394, SPy0128 from M1 strain SF370, SpyM3_{—}0100 from M3 strain 315, SPs0102 from M3 strain SSI, orf80 from M5 isolate Manfredo, spyM18_{—}0128 from M18 strain 8232, SpyoM01000153 from M49 strain 591, 19224137 from M12 strain A735, Fimbrial structural subunit from M77 strain ISS4959, fimbrial structural subunit from M44 strain ISS3776, fimbrial structural subunit from M50 strain ISS3776 ISS 4538, fimbrial structural subunit from M12strain CDC SS635, fimbrial structural subunit from M23 strain DSM2071, fimbrial structural subunit from M6 strain CDC SS410).

TABLE 45
Comparison of Amino Acid Sequences of Major Pilus Proteins in the Four GAS AIs

| | | | | AI-3 | | | |
|---|---|---|---|---|---|---|---|
| | | AI-1 | AI-2 | | M5- | | AI-4 |
| | | M6-10394 | M1-370 | M3-315 | Manfredo | M18-8232 | M12-A735 |
| AI-1 | M6-10394 | 100% | 23% | 25% | 23% | 24% | 26% |
| | | M6-10394 | | | | | |
| | | M6-SS-410 | | | | | |
| | | 56% | | | | | |
| | | M23-DSM2071 | | | | | |
| AI-2 | M1-370 | 23% | 100% | 40% | 41% | 38% | 40% |
| AI-3 | M3-315 | 25% | 40% | 100% | 64% | 67% | 61% |
| | | | | M3-315 | | | |
| | | | | M3-SSI-1 | | | |
| | M5-Manfredo | 23% | 39% | 64% | 100% | 60% | 65% |
| | | | | | M5- | | |
| | | | | | Manfredo | | |
| | | | | | M44-3776 | | |
| | | | | | M77-4959 | | |
| | M18-8232 | 24% | 38% | 67% | 60% | 90% | 62% |
| | | | | | | M18-8232 | |
| | | | | | | M49-591 | |
| AI-4 | M12-A735 | 26% | 40% | 61% | 65% | 62% | 97-100% |
| | | | | | | | M12-A735 |
| | | | | | | | M12-SS-635 |
| | | | | | | | M50-4538 |

For example, the first main pilus subunit may be selected from bacteria of GAS serotype M6 strain 10394 and the second main pilus subunit may be selected from bacteria of GAS serotype M1 strain 370. As can be seen from Table 45, the main pilus subunits encoded by these strains of bacteria share only 23% nucleotide identity. An immunogenic composition comprising pilus main subunits from each of these strains of bacteria is expected to provide protection across a wider group of GAS strains and serotypes. Other examples of main pilus subunits that can be used in combination to provide increased protection across a wider range of GAS strains and serotypes include proteins encoded by GAS serotype M5 Manfredo isolate and serotype M6 strain 10394, which share 23% sequence identity, GAS serotype M18 strain 8232 and serotype M1 strain 370, which share 38% sequence identity, GAS serotype M3 strain 315 and serotype M12 strain A735, which share 61% sequence identity, and GAS serotype M3 strain 315 and serotype M6 strain 10394 which share 25% sequence identity.

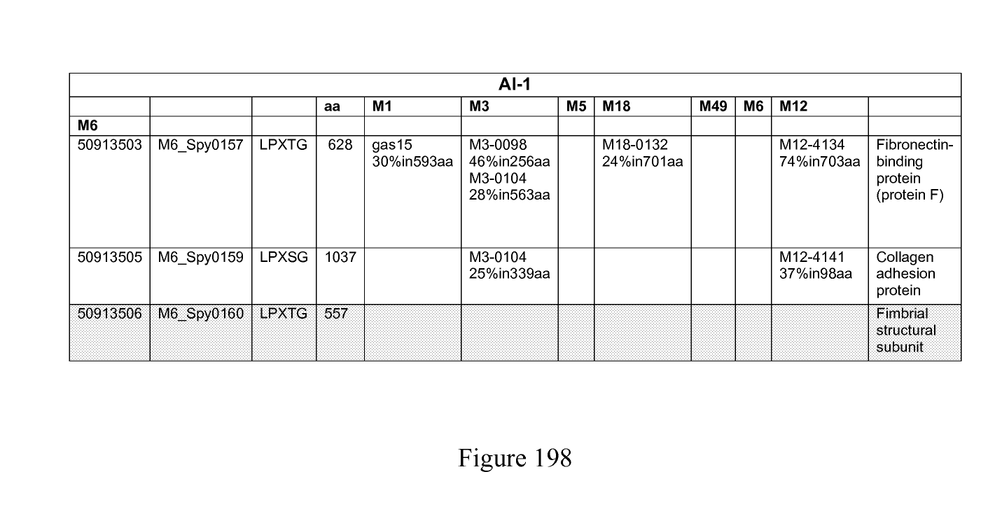

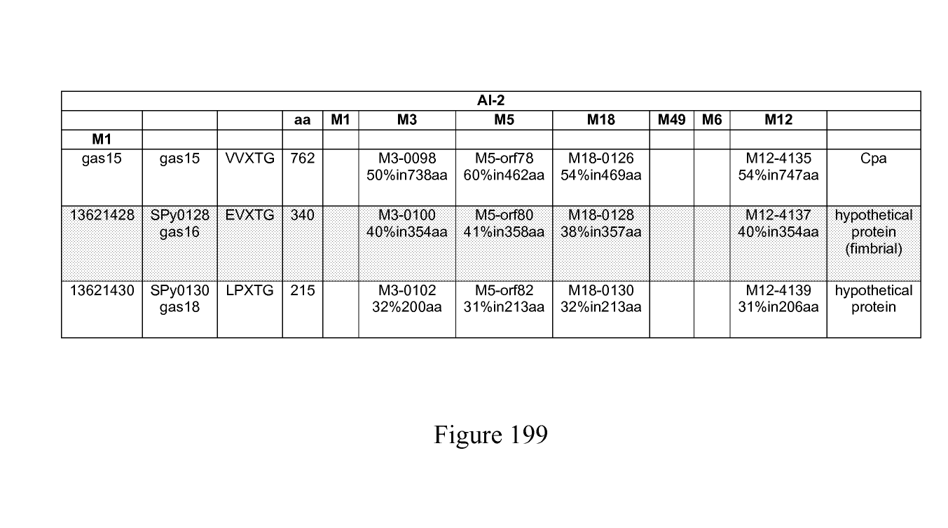

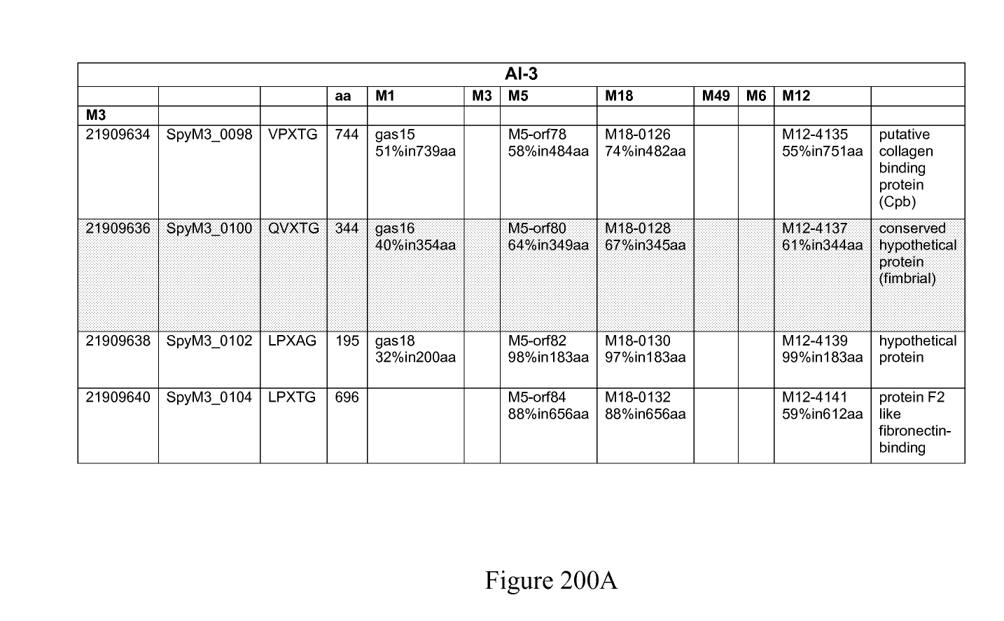

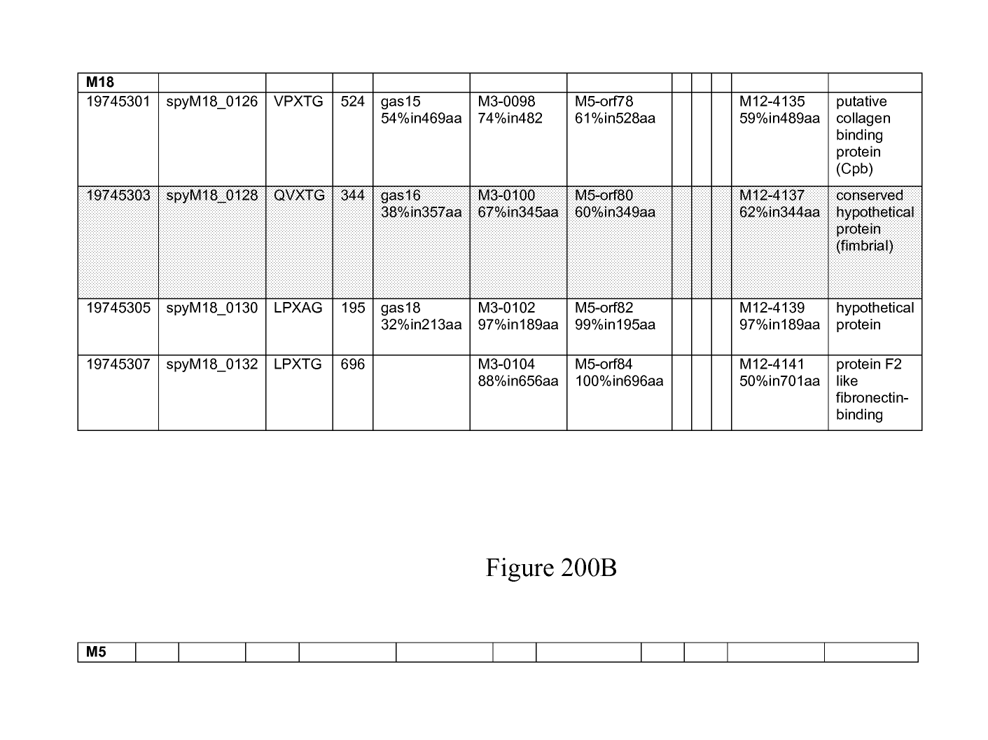

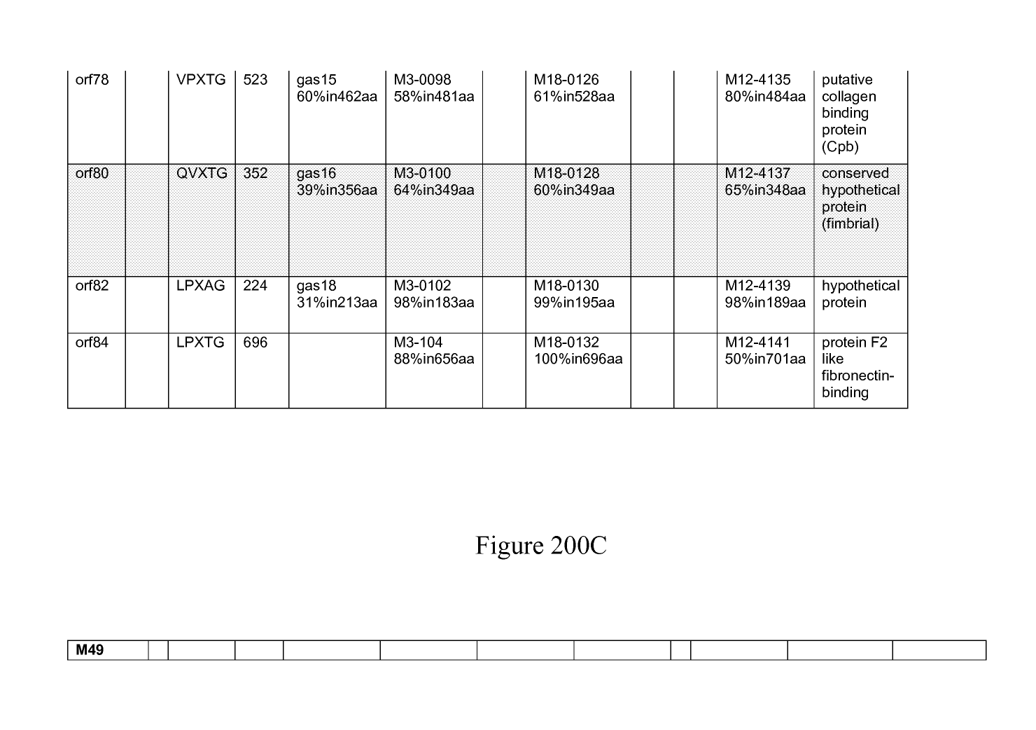

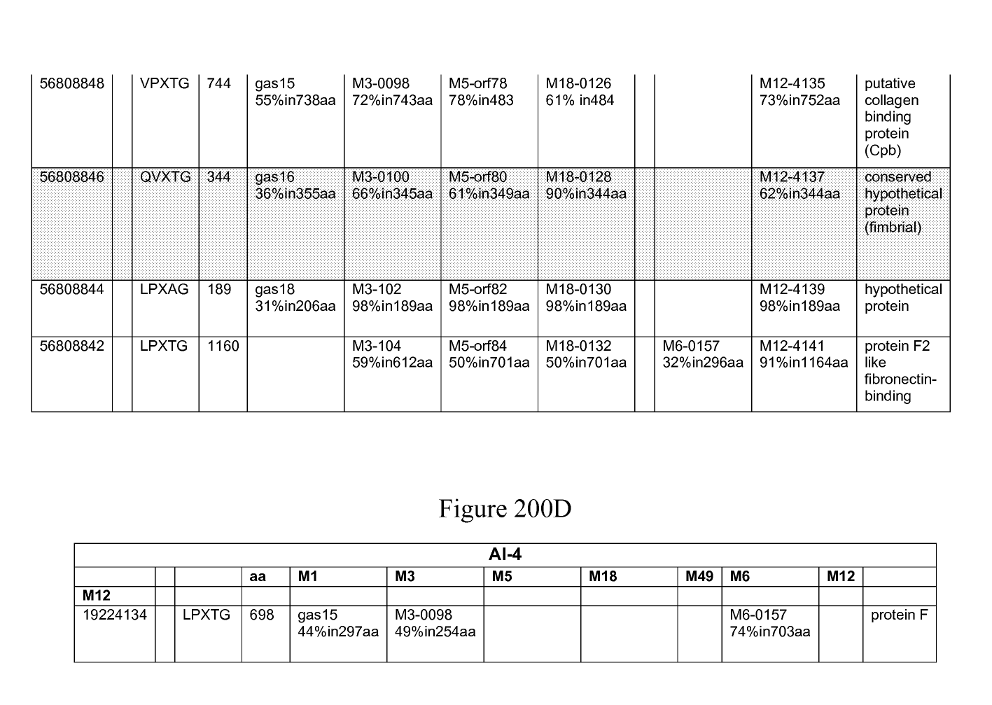

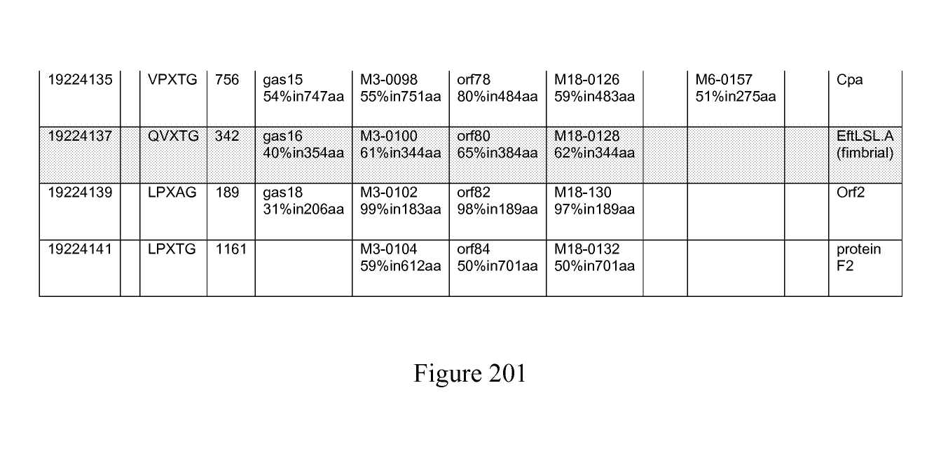

As also can be seen from Table 45, the amino acid sequences of the four types of main pilus subunits present in GAS are relatively divergent. FIGS. 198-201 provide further tables comparing the percent identity of adhesin island-encoded surface exposed proteins for different GAS serotypes relative to other GAS serotypes harbouring an adhesin island of the same or a different subtype (GAS AI-1, GAS AI-2, GAS AI-3, and GAS AI-4). See also further discussion below.

Immunizations with the Adhesin Island proteins of the invention are discussed further in the Examples.

### Co-Expression of GBS Adhesin Island Proteins and Role of GBS AI Proteins in Surface Presentation

In addition to the use of the GBS adhesin island proteins for cross strain and cross serotype protection, Applicants have identified interactions between adhesin island proteins which appear to affect the delivery or presentation of the surface proteins on the surface of the bacteria.

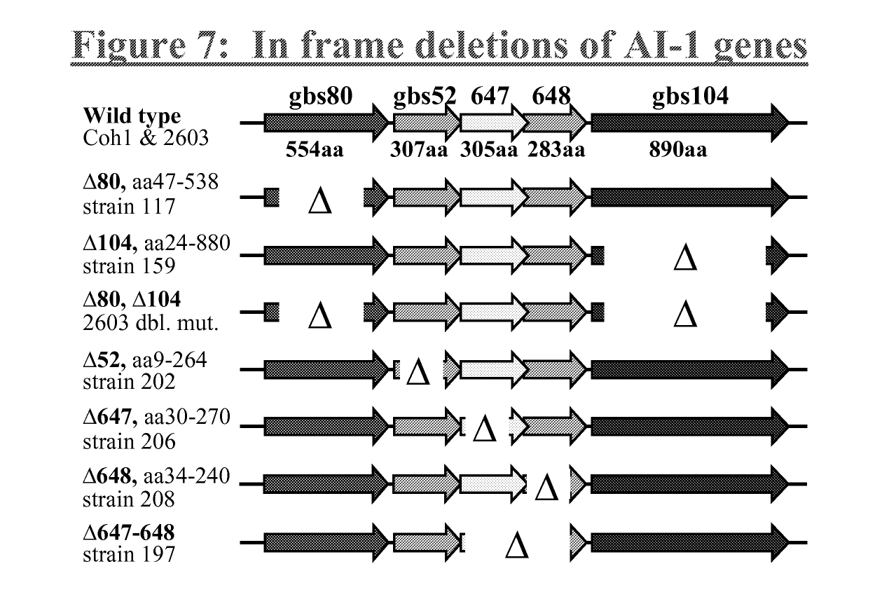

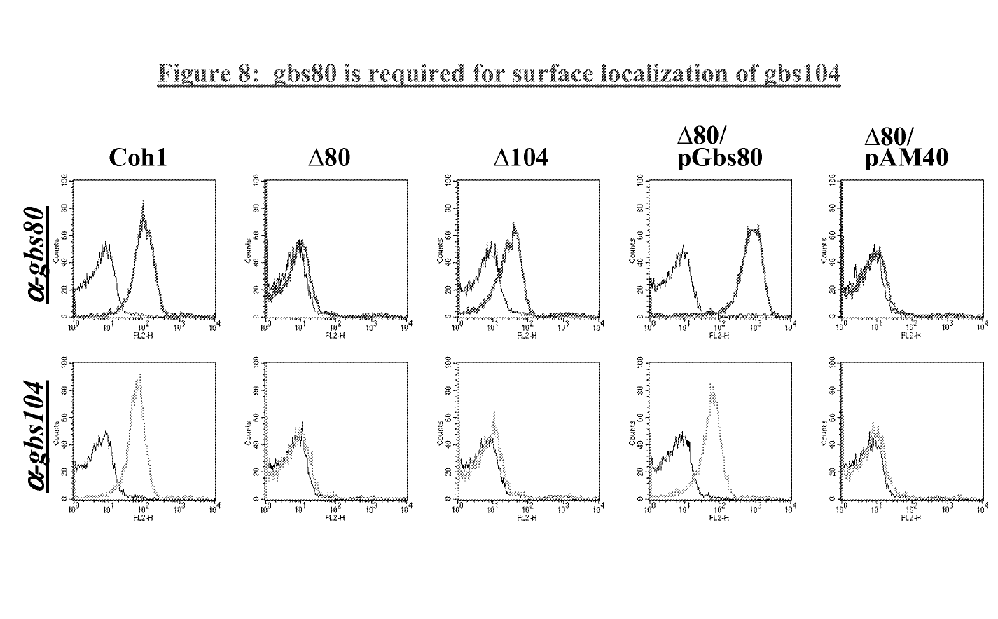

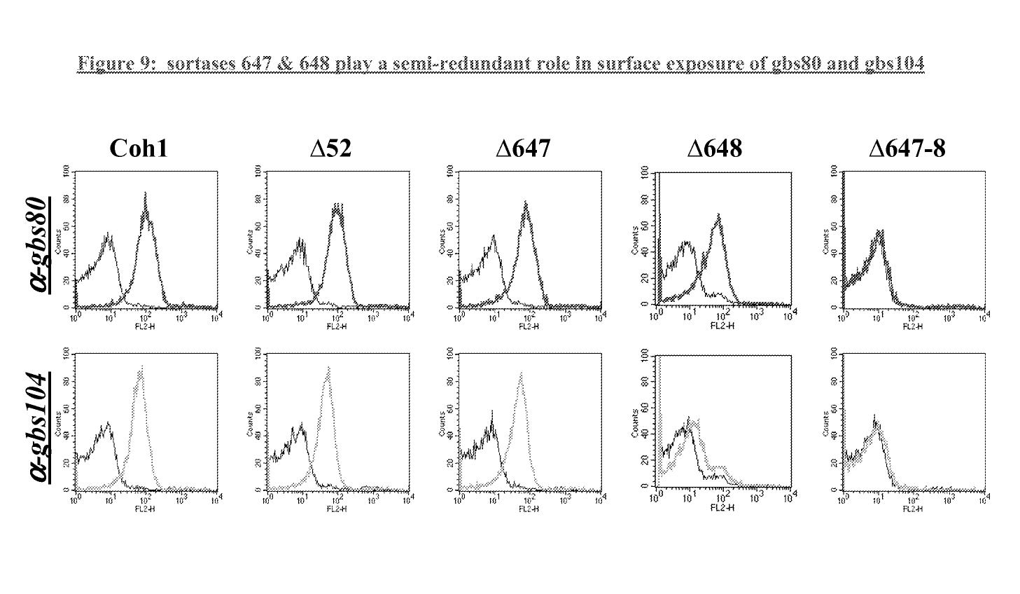

In particular, Applicants have discovered that surface exposure of GBS 104 is dependent on the concurrent expression of GBS 80. As discussed further in Example 2, reverse transcriptase PCR analysis of AI-1 shows that all of the AI genes are co-transcribed as an operon. Applicants constructed a series of mutant GBS containing in frame deletions of various AI-1 genes. (A schematic of the GBS mutants is presented in FIG. 7). FACS analysis of the various mutants comparing mean shift values using anti-GBS 80 versus anti-GBS 104 antibodies is presented in FIG. 8. Removal of the GBS 80 operon prevented surface exposure of GBS 104; removal of the GBS 104 operon did not affect surface exposure of GBS 80. While not being limited to a specific theory, it is thought that GBS 80 is involved in the transport or localization of GBS 104 to the surface of the bacteria. The two proteins may be oligomerized or otherwise associated. It is possible that this association involves a conformational change in GBS 104 that facilitates its transition to the surface of the GBS bacteria.

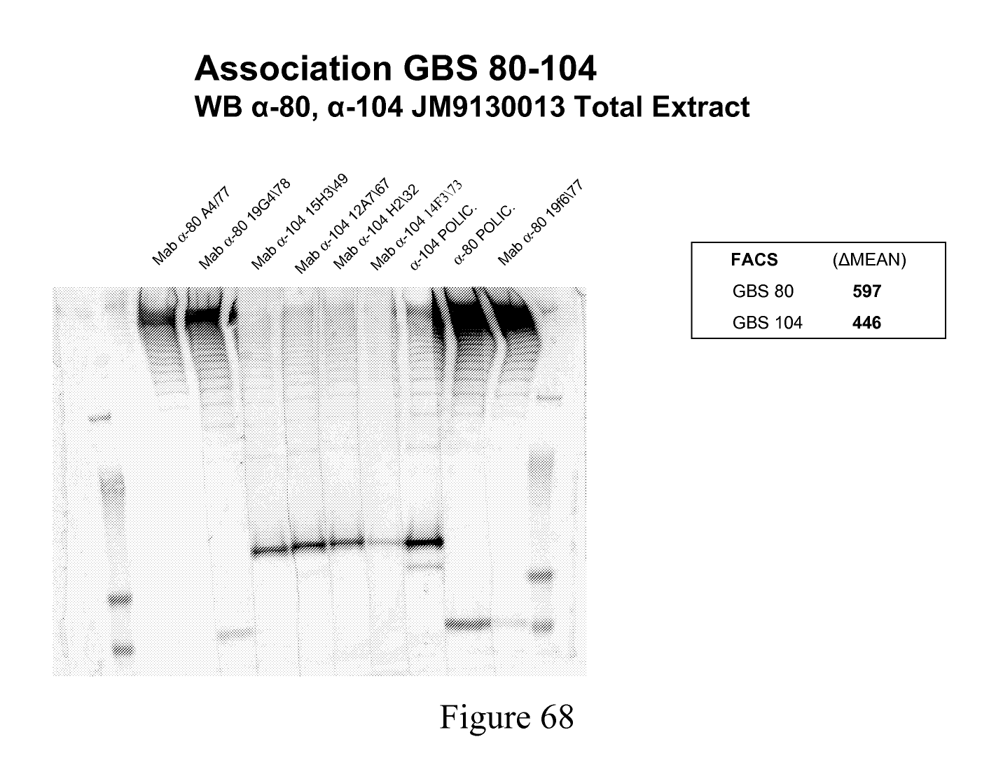

Pili structures that comprise GBS 104 appear to be of a lower molecular weight than pili structures lacking GBS 104. FIG. 68 shows that polyclonal anti-GBS 104 antibodies (see lane marked α-104 POLIC.) cross-hybridize with smaller structures than do polyclonal anti-GBS 80 antibodies (see lane marked α-GBS 80 POLIC.).

In addition, Applicants have shown that removal of GBS 80 can cause attenuation, further suggesting the protein contributes to virulence. As described in more detail in Example 3, the LD₅₀′s for the Δ80 mutant and the Δ80, Δ104 double mutant were reduced by an order of magnitude compared to wildtype and Δ104 mutant.

The sortases within the adhesin island also appear to play a role in localization and presentation of the surface proteins. As discussed further in Example 4, FACS analysis of various sortase deletion mutants showed that removal of sortase SAG0648 prevented GBS 104 from reaching the surface and slightly reduced the surface exposure of GBS 80. When sortase SAG0647 and sortase SAG0648 were both knocked out, neither GBS 80 nor GBS 104 were surface exposed. Expression of either sortase alone was sufficient for GBS 80 to arrive at the bacterial surface. Expression of SAG0648, however, was required for GBS 104 surface localization.

Accordingly, the compositions of the invention may include two or more AI proteins, wherein the AI proteins are physically or chemically associated. For example, the two AI proteins may form an oligomer. In one embodiment, the associated proteins are two AI surface proteins, such as GBS 80 and GBS 104. The associated proteins may be AI surface proteins from different adhesin islands, including host cell adhesin island proteins if the AI surface proteins are expressed in a recombinant system. For example, the associated proteins may be GBS 80 and GBS 67.

Adhesin Island Proteins from Other Gram Positive Bacteria

Applicants' identification and analysis of the GBS adhesin islands and the immunological and biological functions of these AI proteins and their pilus structures provides insight into similar structures in other Gram positive bacteria.

As discussed above, “Adhesin Island” or “AI” refers to a series of open reading frames within a bacterial genome that encode for a collection of surface proteins and sortases. An Adhesin Island may encode for amino acid sequences comprising at least one surface protein. The Adhesin Island may encode at least one surface protein. Alternatively, an Adhesin Island may encode for at least two surface proteins and at least one sortase. Preferably, an Adhesin Island encodes for at least three surface proteins and at least two sortases. One or more of the surface proteins may include an LPXTG motif (e.g., SEQ ID NO:122) or other sortase substrate motif. One or more AI surface proteins may participate in the formation of a pilus structure on the surface of the Gram positive bacteria.

Gram positive adhesin islands of the invention preferably include a divergently transcribed transcriptional regulator. The transcriptional regulator may regulate the expression of the AI operon.

The invention includes a composition comprising one or more Gram positive bacteria AI surface proteins. Such AI surface proteins may be associated in an oligomeric or hyperoligomeric structure.

Preferred Gram positive adhesin island proteins for use in the invention may be derived from Staphylococcus (such as S. aureus), Streptococcus (such as S. agalactiae (GBS), S. pyogenes (GAS), S. pneumoniae, S. mutans), Enterococcus (such as E. faecalis and E. faecium), Clostridium (such as C. difficile), Listeria (such as L. monocytogenes) and Corynebacterium (such as C. diphtheria).

One or more of the Gram positive AI surface protein sequences typically include an LPXTG motif or other sortase substrate motif. Gram positive AI surface proteins of the invention may affect the ability of the Gram positive bacteria to adhere to and invade epithelial cells. AI surface proteins may also affect the ability of Gram positive bacteria to translocate through an epithelial cell layer. Preferably, one or more AI surface proteins are capable of binding to or otherwise associating with an epithelial cell surface. Gram positive AI surface proteins may also be able to bind to or associate with fibrinogen, fibronectin, or collagen.

Gram positive AI sortase proteins are predicted to be involved in the secretion and anchoring of the LPXTG containing surface proteins. A Gram positive bacteria AI may encode for at least one surface exposed protein. The Adhesin Island may encode at least one surface protein. Alternatively, a Gram positive bacteria AI may encode for at least two surface exposed proteins and at least one sortase. Preferably, a Gram positive AI encodes for at least three surface exposed proteins and at least two sortases.

Gram positive AI surface proteins may be covalently attached to the bacterial cell wall by membrane-associated transpeptidases, such as an AI sortase. The sortase may function to cleave the surface protein, preferably between the threonine and glycine residues of an LPXTG motif. The sortase may then assist in the formation of an amide link between the threonine carboxyl group and a cell wall precursor such as lipid II. The precursor can then be incorporated into the peptidoglycan via the transglycoslylation and transpeptidation reactions of bacterial wall synthesis. See Comfort et al., Infection & Immunity (2004) 72(5): 2710-2722. Typically, Gram positive bacteria AI surface proteins of the invention will contain an N-terminal leader or secretion signal to facilitate translocation of the surface protein across the bacterial membrane.

Gram positive bacteria AI surface proteins of the invention may affect the ability of the Gram positive bacteria to adhere to and invade target host cells, such as epithelial cells. Gram positive bacteria AI surface proteins may also affect the ability of the gram positive bacteria to translocate through an epithelial cell layer. Preferably, one or more of the Gram positive AI surface proteins are capable of binding to or other associating with an epithelial cell surface. Further, one or more Gram positive AI surface proteins may bind to fibrinogen, fibronectin, or collagen protein.

In one embodiment, the invention includes a composition comprising oligomeric, pilus-like structures comprising a Gram positive bacteria AI surface protein. The oligomeric, pilus-like structure may comprise numerous units of the AI surface protein. Preferably, the oligomeric, pilus-like structures comprise two or more AI surface proteins. Still more preferably, the oligomeric, pilus-like structure comprises a hyper-oligomeric pilus-like structure comprising at least two (e.g., 2, 3, 4, 5, 6, 7, 8, 9, 10, 11, 12, 13, 14, 15, 20, 25, 30, 35, 40, 45, 50, 60, 70, 80, 90, 100, 120, 140, 150, 200 or more) oligomeric subunits, wherein each subunit comprises an AI surface protein or a fragment thereof. The oligomeric subunits may be covalently associated via a conserved lysine within a pilin motif. The oligomeric subunits may be covalently associated via an LPXTG motif, preferably, via the threonine amino acid residue.

Gram positive bacteria AI surface proteins or fragments thereof to be incorporated into the oligomeric, pilus-like structures of the invention will preferably include one or both of a pilin motif comprising a conserved lysine residue and an E box motif comprising a conserved glutamic acid residue.

The oligomeric, pilus like structures may be used alone or in the combinations of the invention. In one embodiment, the invention comprises a Gram positive bacteria Adhesin Island in oligomeric form, preferably in a hyperoligomeric form.

The oligomeric, pilus-like structures of the invention may be combined with one or more additional Gram positive AI proteins (from the same or a different Gram positive species or genus). In one embodiment, the oligomeric, pilus-like structures comprise one or more Gram positive bacteria AI surface proteins in combination with a second Gram positive bacteria protein. The second Gram positive bacteria protein may be a known antigen, and need not normally be associated with an AI protein.

The oligomeric, pilus-like structures may be isolated or purified from bacterial cultures overexpressing a Gram positive bacteria AI surface protein. The invention therefore includes a method for manufacturing an oligomeric Adhesin Island surface antigen comprising culturing a Gram positive bacteria adapted for increased AI protein expression and isolation of the expressed oligomeric Adhesin Island protein from the Gram positive bacteria. The AI protein may be collected from secretions into the supernatant or it may be purified from the bacterial surface. The method may further comprise purification of the expressed Adhesin Island protein. Preferably, the Adhesin Island protein is in a hyperoligomeric form.

Gram positive bacteria are preferably adapted to increase AI protein expression by at least two (e.g., 2, 3, 4, 5, 8, 10, 15, 20, 25, 30, 35, 40, 45, 50, 60, 70, 80, 90, 100, 125, 150 or 200) times wild type expression levels.

Gram positive bacteria may be adapted to increase AI protein expression by means known in the art, including methods of increasing gene dosage and methods of gene upregulation. Such means include, for example, transformation of the Gram positive bacteria with a plasmid encoding the AI protein. The plasmid may include a strong promoter or it may include multiple copies of the sequence encoding the AI protein. Optionally, the sequence encoding the AI protein within the Gram positive bacterial genome may be deleted. Alternatively, or in addition, the promoter regulating the Gram positive Adhesin Island may be modified to increase expression.

The invention further includes Gram positive bacteria which have been adapted to produce increased levels of AI surface protein. In particular, the invention includes Gram positive bacteria which have been adapted to produce oligomeric or hyperoligomeric AI surface protein. In one embodiment, the Gram positive bacteria of the invention are inactivated or attenuated to permit in vivo delivery of the whole bacteria, with the AI surface protein exposed on its surface.

The invention further includes Gram positive bacteria which have been adapted to have increased levels of expressed AI protein incorporated in pili on their surface. The Gram positive bacteria may be adapted to have increased exposure of oligomeric or hyperoligomeric AI proteins on its surface by increasing expression levels of a signal peptidase polypeptide. Increased levels of a local signal peptidase expression in Gram positive bacteria (such us LepA in GAS) are expected to result in increased exposure of pili proteins on the surface of Gram positive bacteria. Increased expression of a leader peptidase in Gram positive may be achieved by any means known in the art, such as increasing gene dosage and methods of gene upregulation. The Gram positive bacteria adapted to have increased levels of leader peptidase may additionally be adapted to express increased levels of at least one pili protein.

Alternatively, the AI proteins of the invention may be expressed on the surface of a non-pathogenic Gram positive bacteria, such as Streptococcus gordonii (See, e.g., Byrd et al., “Biological consequences of antigen and cytokine co-expression by recombinant Streptococcus gordonii vaccine vectors, ” Vaccine (2002) 20:2197-2205) or Lactococcus lactis (See, e.g., Mannam et al., “Mucosal Vaccine Made from Live, Recombinant Lactococcus lactis Protects Mice against Pharyngeal Infection with Streptococcus pyogenes” Infection and Immunity (2004) 72(6):3444-3450). It has already been demonstrated, above, that L. lactis expresses GBS and GAS AI polypeptides in oligomeric form and on its surface.

Alternatively, the oligomeric, pilus-like structures may be produced recombinantly. If produced in a recombinant host cell system, the Gram positive bacteria AI surface protein will preferably be expressed in coordination with the expression of one or more of the AI sortases of the invention. Such AI sortases will facilitate oligomeric or hyperoligomeric formation of the AI surface protein subunits.

Gram positive AI Sortases of the invention will typically have a signal peptide sequence within the first 70 amino acid residues. They may also include a transmembrane sequence within 50 amino acid residues of the C terminus. The sortases may also include at least one basic amino acid residue within the last 8 amino acids. Preferably, the sortases have one or more active site residues, such as a catalytic cysteine and histidine.

Adhesin island surface proteins from two or more Gram positive bacterial genus or species may be combined to provide an immunogenic composition for prophylactic or therapeutic treatment of disease or infection of two more Gram positive bacterial genus or species. Optionally, the adhesin island surface proteins may be associated together in an oligomeric or hyperoligomeric structure.

In one embodiment, the invention comprises an adhesin island surface proteins from two or more Streptococcus species. For example, the invention includes a composition comprising a GBS AI surface protein and a GAS adhesin island surface protein. As another example, the invention includes a composition comprising a GAS adhesin island surface protein and a S. pneumoniae adhesin island surface protein.

In one embodiment, the invention comprises an adhesin island surface protein from two or more Gram positive bacterial genus. For example, the invention includes a composition comprising a Streptococcus adhesin island protein and a Corynebacterium adhesin island protein.

Examples of AI sequences in several Gram positive bacteria are discussed further below.

Streptococcus pyogenes (GAS)

As discussed above, Applicants have identified at least six different GAS Adhesin Islands. These adhesion islands are thought to encode surface proteins which are important in the bacteria's virulence, and Applicants have obtained the first electron micrographs revealing the presence of these adhesin island proteins in hyperoligomeric pilus structures on the surface of Group A Streptococcus.

Group A Streptococcus is a human specific pathogen which causes a wide variety of diseases ranging from pharyngitis and impetigo through life threatening invasive disease and necrotizing fasciitis. In addition, post-streptococcal autoimmune responses are still a major cause of cardiac pathology in children.

Group A Streptococcal infection of its human host can generally occur in three phases. The first phase involves attachment and/or invasion of the bacteria into host tissue and multiplication of the bacteria within the extracellular spaces. Generally this attachment phase begins in the throat or the skin. The deeper the tissue level infected, the more severe the damage that can be caused. In the second stage of infection, the bacteria secrete a soluble toxin that diffuses into the surrounding tissue or even systemically through the vasculature. This toxin binds to susceptible host cell receptors and triggers inappropriate immune responses by these host cells, resulting in pathology. Because the toxin can diffuse throughout the host, the necrosis directly caused by the GAS toxins may be physically located in sites distant from the bacterial infection. The final phase of GAS infection can occur long after the original bacteria have been cleared from the host system. At this stage, the host's previous immune response to the GAS bacteria due to cross reactivity between epitopes of a GAS surface protein, M, and host tissues, such as the heart. A general review of GAS infection can be found in Principles of Bacterial Pathogenesis, Groisman ed., Chapter 15 (2001).

In order to prevent the pathogenic effects associated with the later stages of GAS infection, an effective vaccine against GAS will preferably facilitate host elimination of the bacteria during the initial attachment and invasion stage.

Isolates of Group A Streptococcus are historically classified according to the M surface protein described above. The M protein is surface exposed trypsin-sensitive protein generally comprising two polypeptide chains complexed in an alpha helical formation. The carboxyl terminus is anchored in the cytoplasmic membrane and is highly conserved among all group A streptococci. The amino terminus, which extends through the cell wall to the cell surface, is responsible for the antigenic variability observed among the 80 or more serotypes of M proteins.

A second layer of classification is based on a variable, trypsin-resistant surface antigen, commonly referred to as the T-antigen. Decades of epidemiology based on M and T serological typing have been central to studies on the biological diversity and disease causing potential of Group A Streptococci. While the M-protein component and its inherent variability have been extensively characterized, even after five decades of study, there is still very little known about the structure and variability of T-antigens. Antisera to define T types are commercially available from several sources, including Sevapharma (sevapharma.cz/en).

The gene coding for one form of T-antigen, T-type 6, from an M6 strain of GAS (D741) has been cloned and characterized and maps to an approximately 11 kb highly variable pathogenicity island. Schneewind et al., J Bacteriol. (1990) 172(6):3310-3317. This island is known as the Fibronectin-binding, Collagen-binding T-antigen (FCT) region because it contains, in addition to the T6 coding gene (tee6), members of a family of genes coding for Extra Cellular Matrix (ECM) binding proteins. Bessen et al., Infection & Immunity (2002) 70(3):1159-1167. Several of the protein products of this gene family have been shown to directly bind either fibronectin and/or collagen. See Hanski et al., Infection & Immunity (1992) 60(12):5119-5125; Talay et al., Infection & Immunity (1992(60(9):3837-3844; Jaffe et al. (1996) 12(2):373-384; Rocha et al., Adv Exp Med Biol. (1997) 418:737-739; Kreikemeyer et al., J Biol Chem (2004) 279(16):15850-15859; Podbielski et al., Mol. Microbiol. (1999) 31(4):1051-64; and Kreikemeyer et al., Int. J. Med Microbiol (2004) 294(2-3):177-88. In some cases direct evidence for a role of these proteins in adhesion and invasion has been obtained.

Applicants raised antiserum against a recombinant product of the tee6 gene and used it to explore the expression of T6 in M6 strain 1553650. In immunoblot of mutanolysin extracts of this strain, the antiserum recognized, in addition to a band corresponding to the predicted molecular mass of the tee6 gene product, very high molecular weight ladders ranging in mobility from about 100 kDa to beyond the resolution of the 3-8% gradient gels used. See FIG. 163A, last lane labeled “M6_Tee6.”

This pattern of high molecular weight products is similar to that observed in immunoblots of the protein components of the pili identified in Streptococcus agalactiae (described above) and previously in Corynebacterium diphtheriae. Electron microscopy of strain M6 1553650 with antisera specific for the product of tee6 revealed abundant surface staining and long pilus like structures extending up to 700 nanometers from the bacterial surface, revealing that the T6 protein, one of the antigens recognized in the original Lancefield serotyping system, is located within a GAS Adhesin Island (GAS AI-1) and forms long covalently linked pilus structures. See FIG. 163I.

In addition to the tee6 gene, the FCT region in M6_ISS3650 (GAS AI-1) contains two other genes (prtF1 and cpa) predicted to code for surface exposed proteins; these proteins are characterized as containing the cell wall attachment motif LPXTG. Western blot analysis using antiserum specific for PrtF1 detected a single molecular species with electrophoretic mobility corresponding to the predicted molecular mass of the protein and one smaller band of unknown origin. Western blot analysis using antisera specific for Cpa recognized a high molecular weight covalently linked ladder (FIG. 163A, second lane) Immunogold labelling of Cpa with specific antiserum followed by transmission electron microscopy detected an abundance of Cpa at the cell surface and only occasional structures extending from the cell surface (FIG. 163J).

Four classes of FCT region can be discerned by the types and order of the genes contained within the region. The FCT region of strains of types M3, M5, M18 and M49 have a similar organization whereas those of M6, M1 and M12 differ. See FIG. 164. As discussed below, these four FCT regions correlate to four GAS Adhesin Island types (AI-1, AI-2, AI-3 and AI-4).

Applicants discovery of genes coding for pili in the FCT region of strain M6_{—}1553650 prompted them to examine the predicted surface exposed proteins in the variant FCT regions of three other GAS strains of having different M-type (M1_SF370, M5_ISS4883 and M12_{—}20010296) representing the other three FCT variants. Each gene present in the FCT region of each bacteria was cloned and expressed. Antisera specific for each recombinant protein was then used to probe mutanolysin extracts of the respective strains (6). In M1 strain SF370, there are three predicted surface proteins (Cpa (also referred to as M1_{—}126 and GAS 15), M1_{—}128 (a fimbrial protein also referred to as Spy0128 and GAS 16), and M1_{—}130 (also referred to as Spy0130 and GAS 18)) (GAS AI-2). Antisera specific for each surface protein reacted with a ladder of high molecular weight material (FIG. 163B) Immunogold staining of M1 strain SF370 with antiserum specific for M1_{—}128 revealed pili structures similar to those seen when M6 strain ISS3650 was immunogold stained with antiserum specific for tee6 (See FIG. 1163K). Antisera specific for surface proteins Cpa and M1_{—}130 revealed abundant surface staining and occasional structures extending from the surface of M1 strain SF370 bacteria (FIG. 163S).

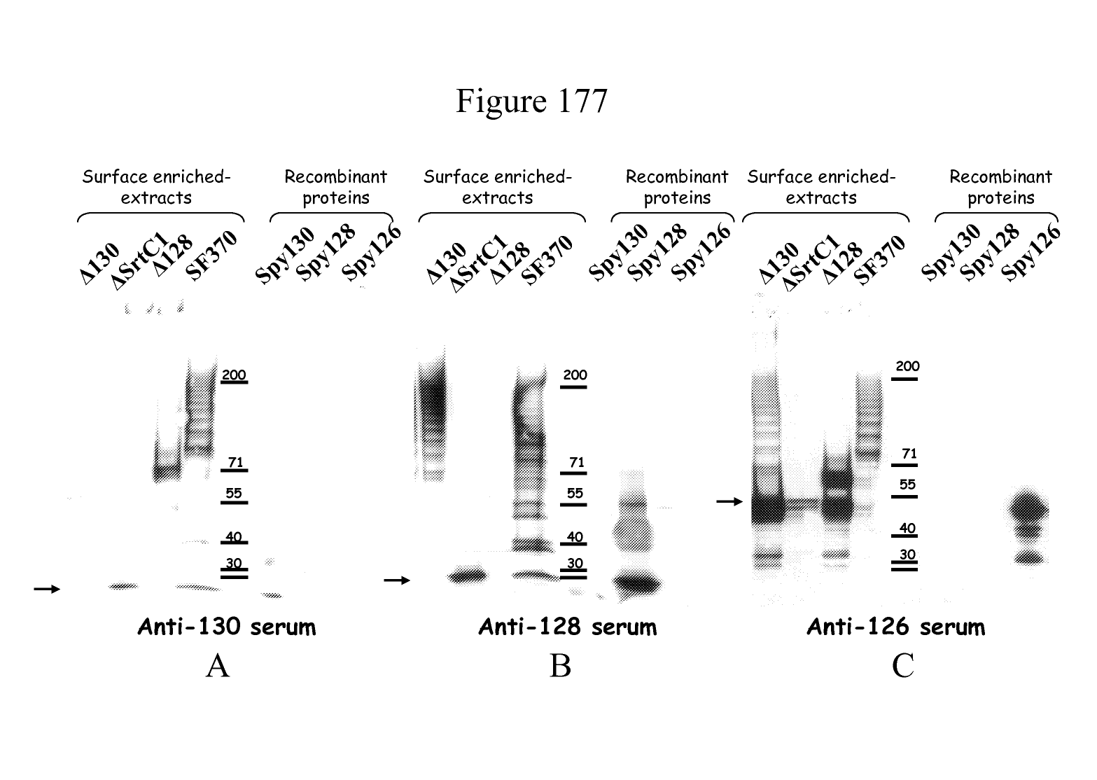

The M1_{—}128 protein appears to be necessary for polymerization of Cpa and M1_{—}130 proteins. If the M1_{—}128 gene in M1_SF370 was deleted, Western blot analysis using antibodies that hybridize to Cpa and M1_{—}130 no longer detected high molecular weight ladders comprising the Cpa and M1_{—}130 proteins (FIG. 163E). See also FIGS. 177A-C which provide the results of Western blot analysis of the M1_{—}128 (Δ128) deleted bacteria using anti-M1_{—}130 antiserum (FIG. 177A), anti-M1_{—}128 antiserum (FIG. 177B), and anti-M1_{—}126 antiserum (FIG. 177C). High molecular weight ladders, indicative of pilus formation on the surface of M1 strain SF370, could not be detected by any of the three antisera in Δ128 bacteria. If the Δ128 bacteria were transformed with a plasmid containing the gene for M1_{—}128, Western blot analysis using antisera specific for Cpa and M1_{—}130 again detected high molecular weight ladders (FIG. 163H).

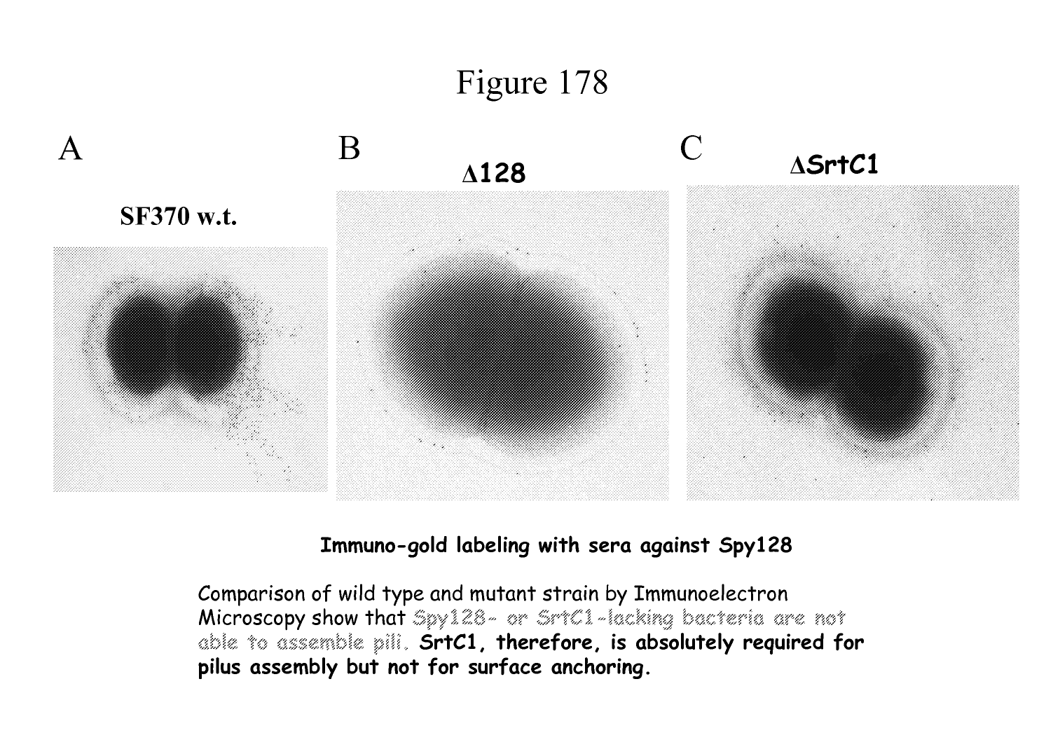

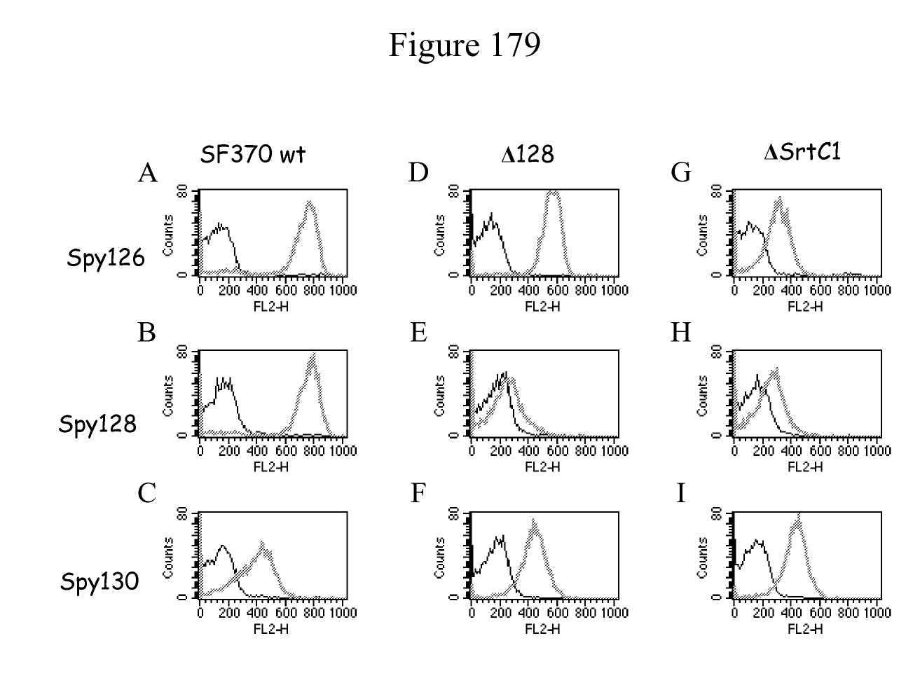

In agreement with the Western blot analysis, immunoelectron microscopy failed to detect pilus assembly on the Δ128 strain SF370 bacteria using M1_{—}128 antisera (FIG. 178B). Although Δ128 SF370 bacteria were unable to form pili, M1_{—}126 (cpa) and M1_{—}130, which contain sortase substrate motifs, were present on the bacteria's surface. FACS analysis of the M1_{—}128 deleted (Δ128) strain SF370 bacteria also detected both M1_{—}126 and M1_{—}130 on the surface of the Δ128 strain SF370 bacteria. See FIGS. 179D and F, which show a shift in fluorescence when antibodies immunoreactive to M1_{—}126 and M1_{—}130 are used on Δ128 bacteria. As expected, virtually no shift in fluorescence is observed when antibodies immunoreactive to M1_{—}128 are used with the Δ128 bacteria (FIG. 179E).

By contrast, deletion of the M1_{—}130 gene did not effect polymerization of M1_{—}128 (FIG. 163F). See also FIGS. 177A-C, which provide Western blot analysis results of the M1_{—}130 deleted (Δ130) strain SF370 bacteria using anti-M1_{—}130 (FIG. 177A), anti-M1_{—}128 (FIG. 177B), and anti-M1_{—}126 antiserum (FIG. 177C). The anti-M1_{—}128 and anti-M1_{—}126 antiserum both detected the presence of high molecular weight ladders in the Δ130 strain SF370 bacteria, indicating that the Δ130 bacteria form pili that comprise M1_{—}126 and M1_{—}128 polypeptides in the absence of M1_{—}130. As expected, the Western blot probed with antiserum immunoreactive with M1_{—}130 did not detect any proteins for the Δ130 bacteria (FIG. 177A).

Hence, the composition of the pili in GAS resembles that previously described for both C. diphtheria (7, 8) and S. agalactiae (described above) (9) in that each pilus is formed by a backbone component which abundantly stains the pili in EM and is essential for the incorporation of the other components.

Also similar to C. diphtheria, elimination of the srtC1 gene from the FCT region of M1_SF370 abolished polymerization of all three proteins and assembly of pili (FIG. 163G). See also FIGS. 177A-C, which provide Western blot analysis of the SrtC1 deleted (ASrtC1) strain SF370 bacteria using anti-M1_{—}130 (FIG. 177A), anti-M1_{—}128 (FIG. 177B), and anti-M1_{—}126 antiserum (FIG. 177C). None of the three antisera immunoreacted with high molecular weight structures (pili) in the ΔSrtC1 bacteria. Confirming that deletion of the SrtC1 gene abrogates pilus assembly in strain SF370, immunoelectron microscopy using antisera against M1_{—}128 failed to detect pilus formation on the bacteria surface. See FIG. 178C. Although no assembled pili were detected on ΔSrtC1 SF370, M1_{—}128 proteins could be detected on the surface of SF370. Thus, it appeared that SrtC1 deletion prevented pilus assembly on the surface of the SF370 bacteria, but not anchoring of the proteins that comprise pili to the bacterial cell wall. FACS analysis of the ΔSrtC1 strain SF370 confirmed that deletion of SrtC1 does not eliminate cell surface expression of M1_{—}126, M1_{—}128 or M1_{—}130. See FIG. 179G-I, which show a shift in fluorescence when antibodies immunoreactive to M1_{—}126 (FIG. 179G), M1_{—}128 (FIGS. 179H), and M1_{—}130 (FIG. 179I) are used to detect cell surface protein expression on ΔSrtC1 bacteria. Thus, SrtC1 deletion prevents pilus formation, but not surface anchoring of proteins involved in pilus formation on the surface of bacteria. Another sortase is possibly involved in anchoring of the proteins to the bacteria surface. Pilus polymerization in C. diphtheriae is also dependent on particular sortase enzyme whose gene resides at the same genetic locus as the pilus components (7, 8).

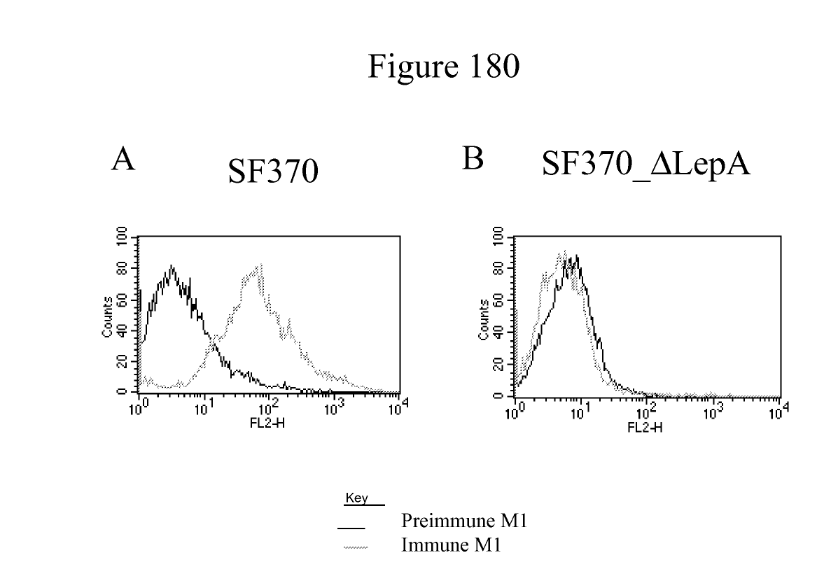

The LepA signal peptidase, Spy0127, also appears to be essential for pilus assembly in strain SF370. LepA deletion mutants (ΔLepA) of strain SF370 fail to assemble pili on the cell surface. Not only are the ΔLepA mutants unable to assemble pili, they are also deficient at cell surface M1 expression. See FIG. 180, which provides a FACS analysis of the wildtype (A) and ALepA mutant (B) SF370 bacteria using M1 antisera. No shift in fluorescence is observed for the ΔLepA mutant bacteria in the presence of M1 immune serum. It is possible that these deletion mutants of LepA will be useful for detecting non-M, non-pili, surface exposed antigens on the surface of GAS, or any Gram positive bacteria. These antigens may also be useful in immunogenic compositions.

Pili were also observed in M5 strain ISS4882 and M12 strain 20010296. The M5 strain ISS4882 contains genes for four predicted surface exposed proteins (GAS AI-3). Antisera against three of the four products of the FCT region (GAS AI-3) of M5_ISS4883 (Cpa, M5_orf80, M5_orf82) stained high molecular weight ladders in Western blot analysis (FIG. 163C). Long pili were visible when antisera against M5_orf80 was used in immunogold staining followed by electron microscopy (FIG. 163L).

The M12 strain 20010296 contains genes for five predicted surface exposed proteins. (GAS AI-4) Antisera against three of the five products of the FCT region (GAS AI-4) of M12_{—}20010296 (Cpa, EftLSL.A, Orf2) stained high molecular weight ladders in Western blot analysis (FIG. 163D). Long pili were visible when antisera against EftLSL.A were used (FIG. 163M).

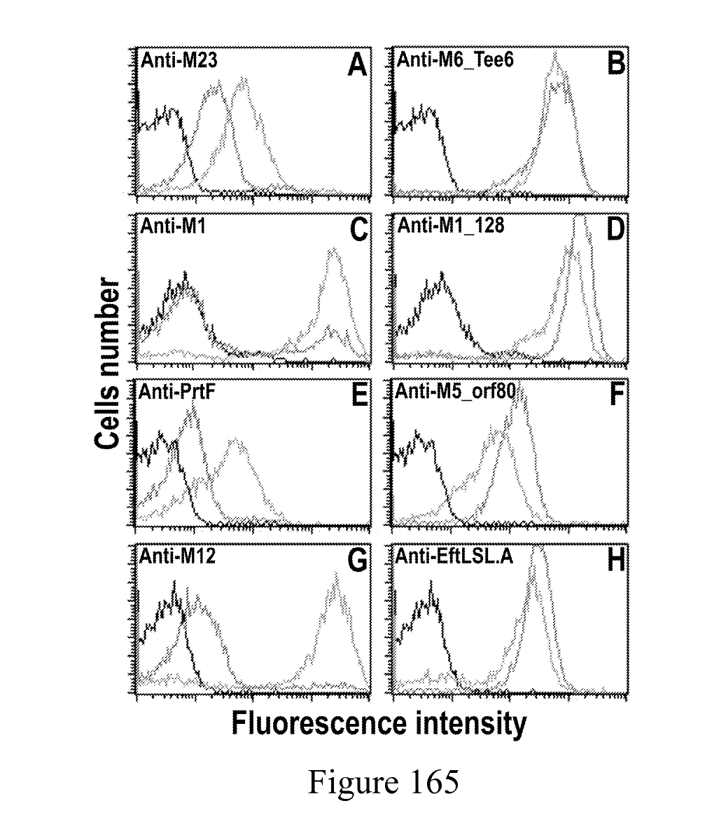

The major pilus forming proteins identified in the four strains studied by applicants (T6, M1_{—}128, M5_orf80 and EftLSL.A) share between 23% and 65% amino acid identity in any pairwise comparison, indicating that each pilus may represent a different Lancefield T-antigen. Each pilus is part of a trypsin resistant structure on the GAS bacteria surface, as is the case for the Lancefield T antigens. See FIG. 165, which provides a FACS analysis of bacteria harboring each of the FCT types that had or had not been treated with trypsin (6). Following treatment, surface expression of the pilus proteins was assayed by indirect immunofluorescence and flow cytometry using antibodies specific for the pilus proteins, the bacteria's respective M proteins, or surface proteins not associated with the pili (FIG. 165). Staining the cells with sera specific for proteins associated with the pili was not effected by trypsin treatment, whereas trypsin treatment substantially reduced detection of M-proteins or surface proteins not associated with pili.

The pili structures identified on the surface of the GAS bacteria were confirmed to be Lancefield T antigens when commercially available T-serotyping sera detected the pili on the surface of bacteria. Western blot analysis was initially performed to determine if polyvalent serum pools (designated T, U, W, X, and Y) could detect recombinant proteins for each of the major pilus components (T6, M1_{—}128, M5_orf80 and EftLSL.A) identified in the strains of bacteria discussed above. Pool U, which contains the T6 serum, recognized the T6 protein specifically (a surface exposed pilus protein from GAS AI-1)(FIG. 166B). Pool T specifically recognized M1_{—}128 (a surface exposed pilus protein from GAS AI-2) (FIG. 166A). Pool W recognized both M5_orf80 and EftLSL.A (FIG. 166C). Using monovalent sera representative of each of the components of each polyvalent pool, applicants confirmed the specificity of the T6 antigen (corresponding to a surface exposed pilus protein from GAS AI-1)(FIG. 166E) and identified M1_{—}128 as antigen T1 (corresponding to a surface exposed pilus protein from GAS AI-2) (FIG. 166D), EftLSL.A as antigen T12 (corresponding to a surface exposed pilus protein from GAS AI-4) (FIGS. 166G) and M5_orf80 as a common antigen recognized by the related sera T5, T27 and T44 (corresponding to a surface exposed pilus protein from GAS AI-3).

Confirming applicants observations, discussed above, that deleting the M1_{—}128 gene from M1_SF370 abolishes pilus formation, the pool T sera stained whole M1_SF370 bacteria (FIG. 166H) but failed to stain M1_SF370 bacteria lacking the M1_{—}128 gene (FIG. 166I).

As discussed above, Applicants have identified at least six different Group A Streptococcus Adhesin Islands. While these GAS AI sequences can be identified in numerous M types, Applicants have surprisingly discovered a correlation between the four main pilus subunits from the four different GAS AI types and specific T classifications. While other trypsin-resistant surface exposed proteins are likely also implicated in the T classification designations, the discovery of the role of the GAS adhesin islands (and the associated hyper-oligomeric pilus like structures) in T classification and GAS serotype variance has important implications for prevention and treatment of GAS infections. Applicants have identified protein components within each of the GAS adhesin islands which are associated with the pilus formation. These proteins are believed to be involved in the bacteria's initial adherence mechanisms Immunological recognition of these proteins may allow the host immune response to slow or prevent the bacteria's transition into the more pathogenic later stages of infection. In addition, the GAS pili may be involved in formation of biofilms. Applicants have discovered that the GBS pili structures appear to be implicated in the formation of biofilms (populations of bacteria growing on a surface, often enclosed in an exopolysaccharide matrix). Biofilms are generally associated with bacterial resistance, as antibiotic treatments and host immune response are frequently unable to eradicate all of the bacteria components of the biofilm. Direction of a host immune response against surface proteins exposed during the first steps of bacterial attachment (i.e., before complete biofilm formation) is preferable.

The invention therefore provides for improved immunogenic compositions against GAS infection which may target GAS bacteria during their initial attachment efforts to the host epithelial cells and may provide protection against a wide range of GAS serotypes. The immunogenic compositions of the invention include GAS AI surface proteins which may be formulated in an oligomeric, or hyperoligomeric (pilus) form. The invention also includes combinations of GAS AI surface proteins. Combinations of GAS AI surface proteins may be selected from the same adhesin island or they may be selected from different GAS adhesin islands.

The invention comprises compositions comprising a first GAS AI protein and a second GAS AI protein wherein the first and second GAS AI proteins are derived from different GAS adhesin islands. For example, the invention includes a composition comprising at least two GAS AI proteins wherein the GAS AI proteins are encoded by the adhesin islands selected from the group consisting of GAS AI-1 and AI-2; GAS AI-1 and GAS AI-3; GAS AI-1 and GAS AI-4; GAS AI-2 and GAS AI-3; GAS AI-2 and GAS AI-4; and GAS AI-3 and GAS AI-4. Preferably the two GAS AI proteins are derived from different T-types.

A schematic arrangement of GAS Adhesin Island sequences is set forth in FIG. 162. In all strains, the AI region is flanked by the highly conserved open reading frames M1_{—}123 and M1-136. Between three and five genes in each locus code for surface proteins containing LPXTG motifs. These surface proteins also all belong to the family of genes coding for ECM binding adhesins.

Adhesin island sequences can be identified in numerous M types of Group A Streptococcus. Examples of AI sequences within M1, M6, M3, M5, M12, M18, and M49 serotypes are discussed below.

GAS Adhesin Islands generally include a series of open reading frames within a GAS genome that encode for a collection of surface proteins and sortases. A GAS Adhesin Island may encode for amino acid sequences comprising at least one surface protein. Alternatively, a GAS Adhesin Island may encode for at least two surface proteins and at least one sortase. Preferably, a GAS Adhesin Island encodes for at least three surface proteins and at least two sortases. One or more of the surface proteins may include an LPXTG motif (e.g., SEQ ID NO:122) or other sortase substrate motif. One or more GAS AI surface proteins may participate in the formation of a pilus structure on the surface of the Gram positive bacteria.

GAS Adhesin Islands of the invention preferably include a divergently transcribed transcriptional regulator. The transcriptional regulator may regulate the expression of the GAS AI operon. Examples of transcriptional regulators found in GAS AI sequences include RofA and Nra.

The GAS AI surface proteins may bind or otherwise adhere to fibrinogen, fibronectin, or collagen. One or more of the GAS AI surface proteins may comprise a fimbrial structural subunit.

One or more of the GAS AI surface proteins may include an LPXTG motif or other sortase substrate motif. The LPXTG motif may be followed by a hydrophobic region and a charged C terminus, which are thought to retard the protein in the cell membrane to facilitate recognition by the membrane-localized sortase. See Barnett, et al., J. Bacteriology (2004) 186 (17): 5865-5875.

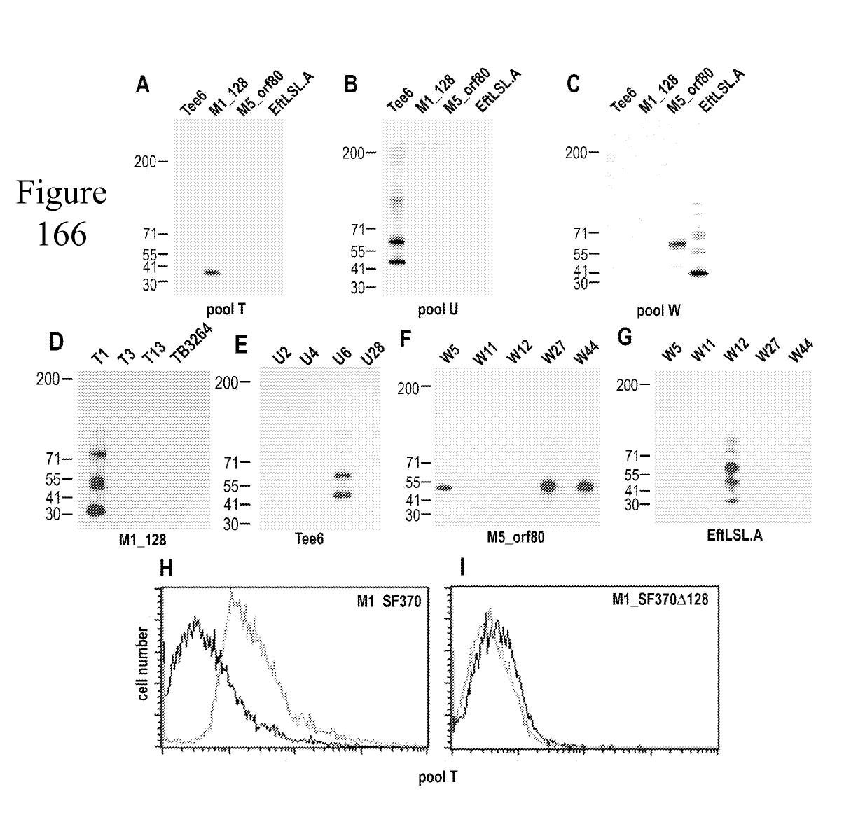

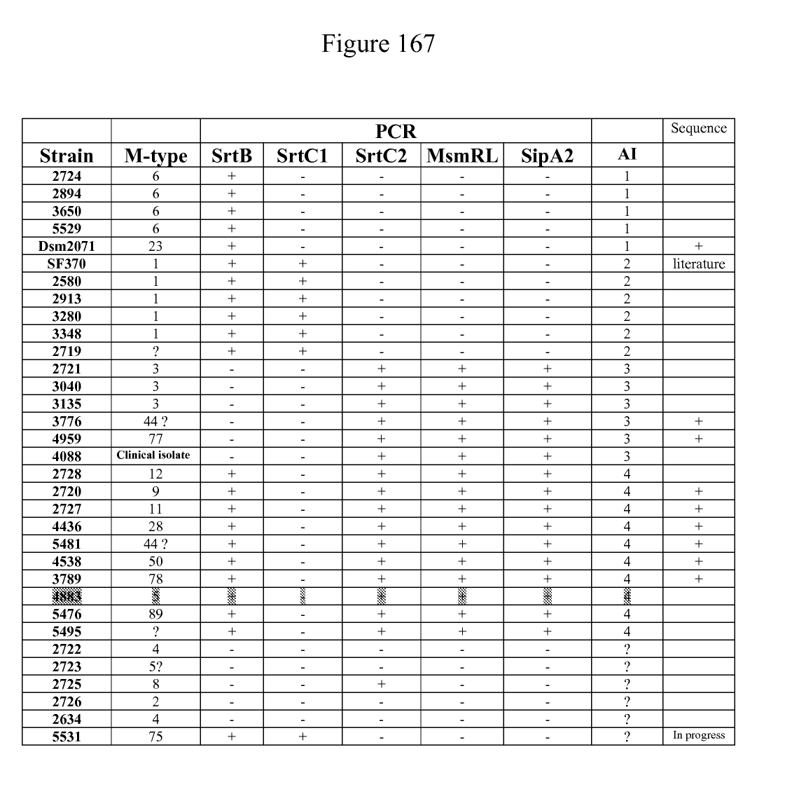

GAS AI sequences may be generally categorized as Type 1, Type 2, Type 3, or Type 4, depending on the number and type of sortase sequences within the island and the percentage identity of other proteins (with the exception of RofA and cpa) within the island. FIG. 167 provides a chart indicating the number and type of sortase sequences identified within the adhesin islands of various strains and serotypes of GAS. As can be seen in this FIG., all GAS strains and serotypes thus far characterized as an AI-1 have a SrtB type sortase, all GAS strains and serotypes thus far characterized as an AI-2 have SrtB and SrtC1 type sortases, all GAS strains and serotypes thus far characterized as an AI-3 have a SrtC2 type sortase, and all GAS strains and serotypes thus far characterized as an AI-4 have SrtB and SrtC2 type sortases. A comparison of the percentage identity of sequences within the adhesin islands was presented in Table 45, see above.

(1) Adhesin Island Sequence Within M6: GAS Adhesin Island 1 (“GAS AI-1”)

A GAS Adhesin Island within M6 serotype (MGAS10394) is outlined in Table 4 below. This GAS adhesin island 1 (“GAS AI-1”) comprises surface proteins, a srtB sortase and a rofA divergently transcribed transcriptional regulator.

GAS AI-1 surface proteins include Spy0157 (a fibronectin binding protein), Spy0159 (a collagen adhesion protein) and Spy0160 (a fimbrial structural subunit). Preferably, each of these GAS AI-1 surface proteins includes an LPXTG sortase substrate motif, such as LPXTG (SEQ ID NO:122) or LPXSG (SEQ ID NO:134) (conservative replacement of threonine with serine).

GAS AI-1 includes a srtB type sortase. GAS srtB sortases may preferably anchor surface proteins with an LPSTG motif (SEQ ID NO:166), particularly where the motif is followed by a serine.

TABLE 4
GAS AI-1 sequences from M6 isolate (MGAS10394)

| | Sortase | |
|---|---|---|
| AI-1 | substrate | |
| sequence | sequence or | |
| identifier | sortase type | functional description |
| M6_Spy0156 | | Transcriptional regulator (rofA) |
| M6_Spy0157 | LPXTG | Fibronectin-binding protein |
| M6_Spy0158 | | Reverse transcriptase |
| M6_Spy0159 | LPXSG | Collagen adhesion protein |
| M6_Spy0160 | LPXTG | Fimbrial structural subunit |
| M6_Spy0161 | srtB | Sortase |

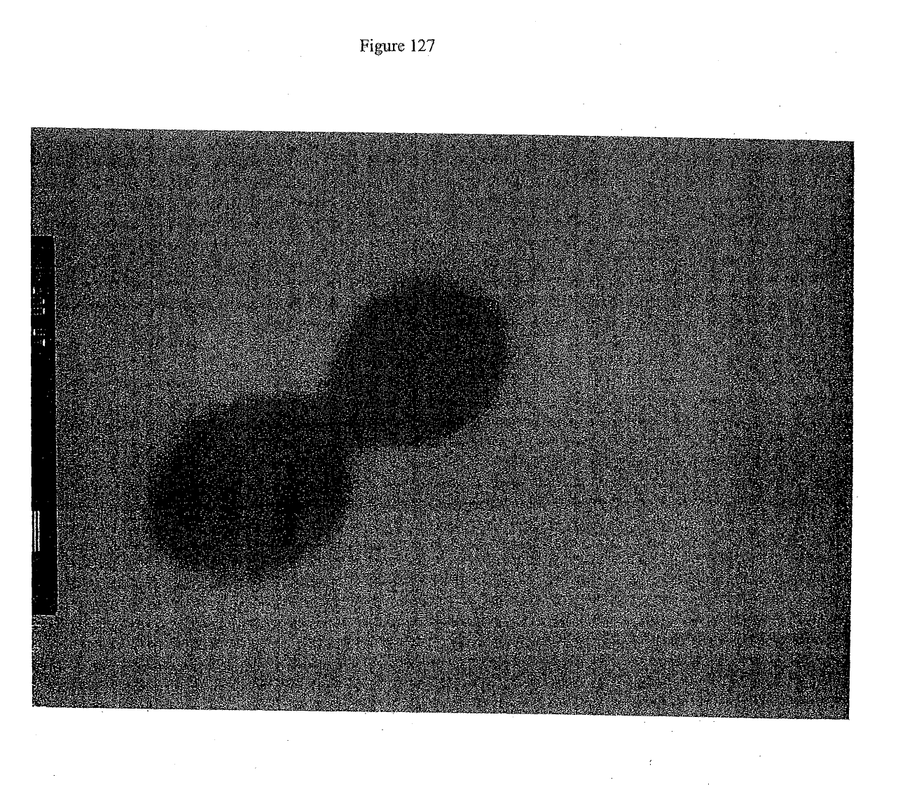

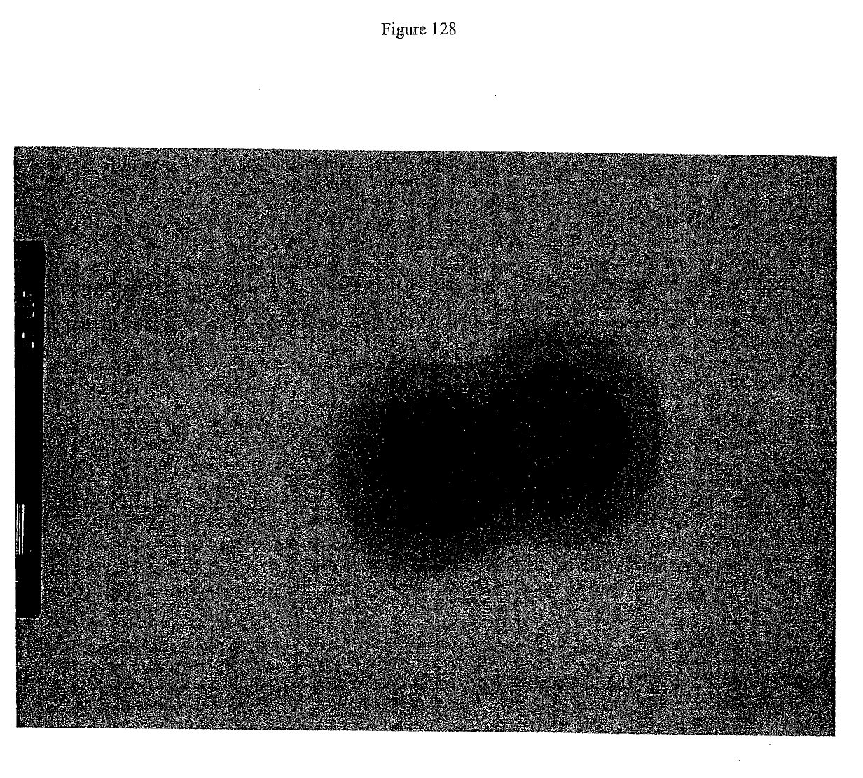

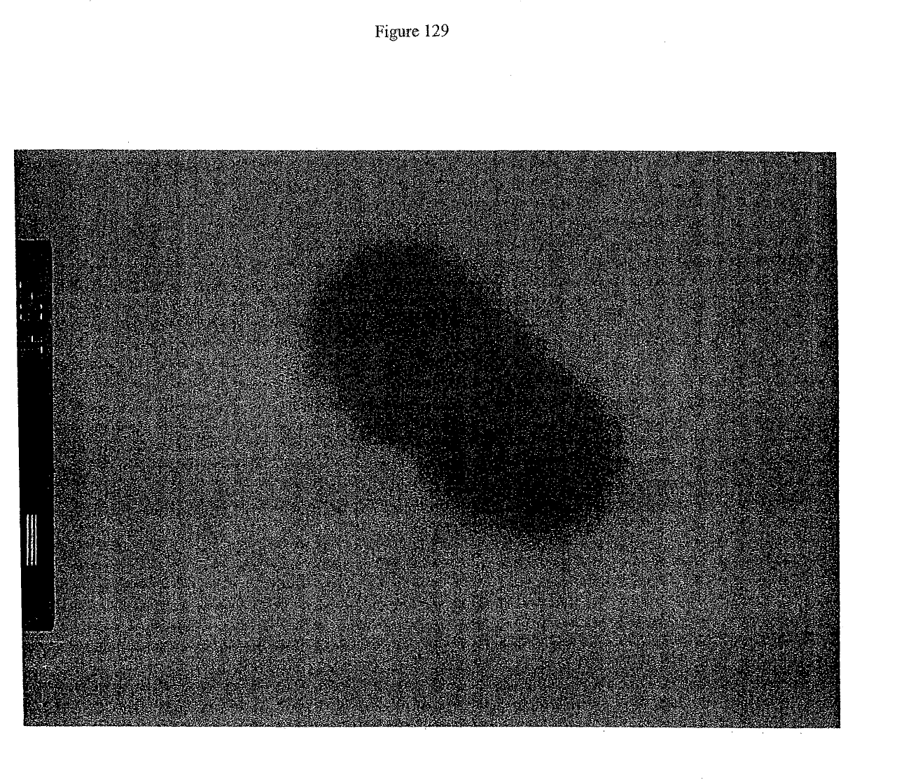

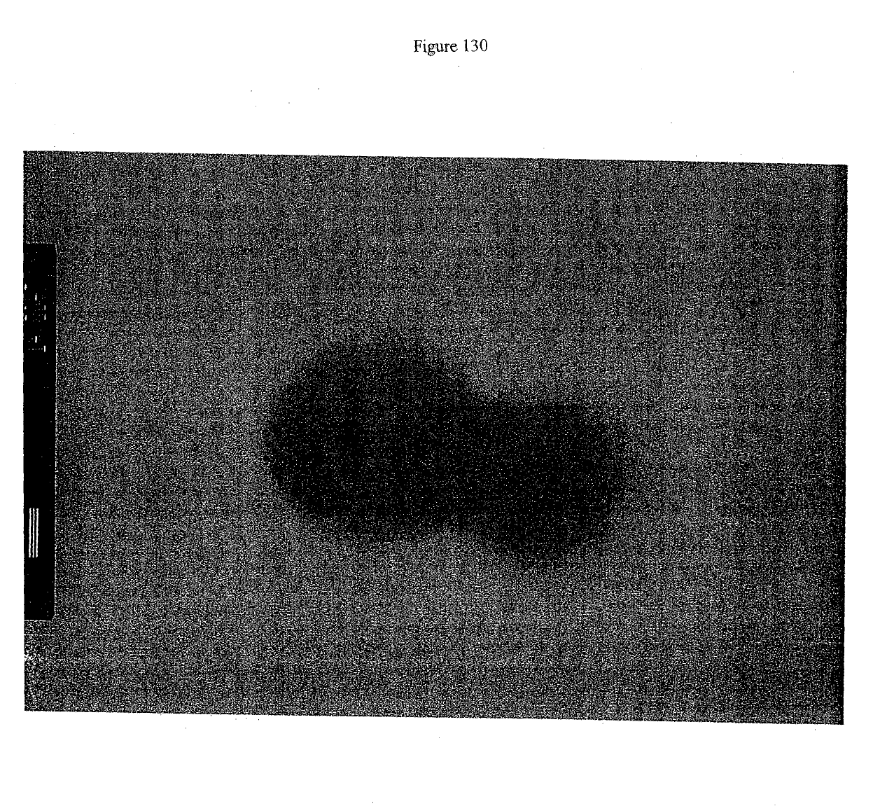

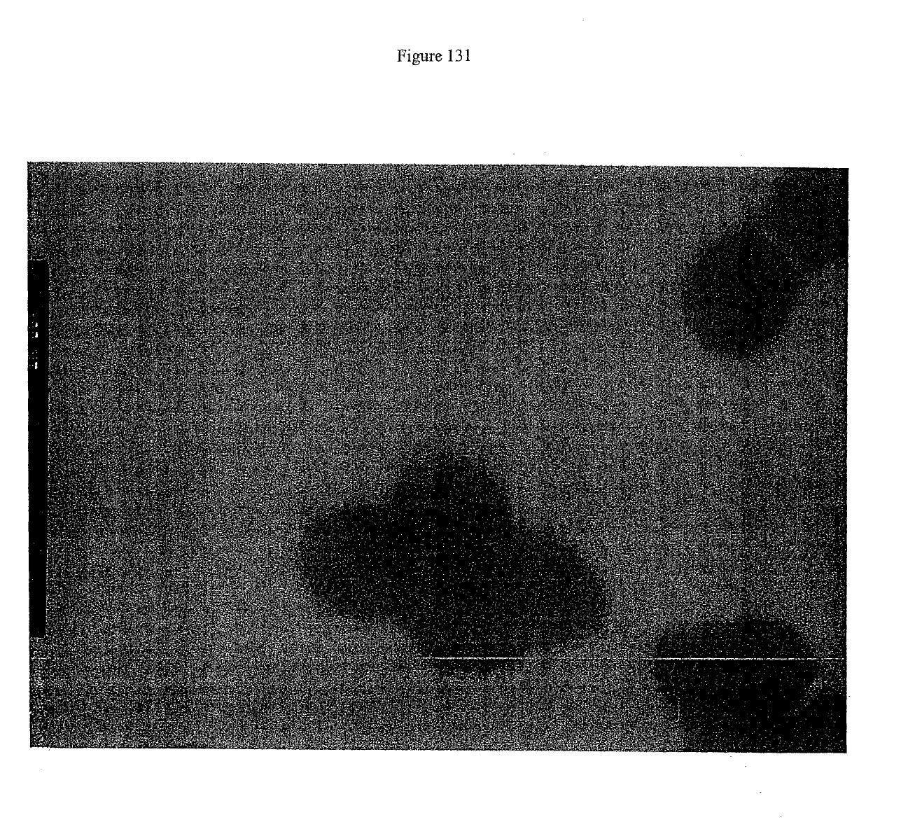

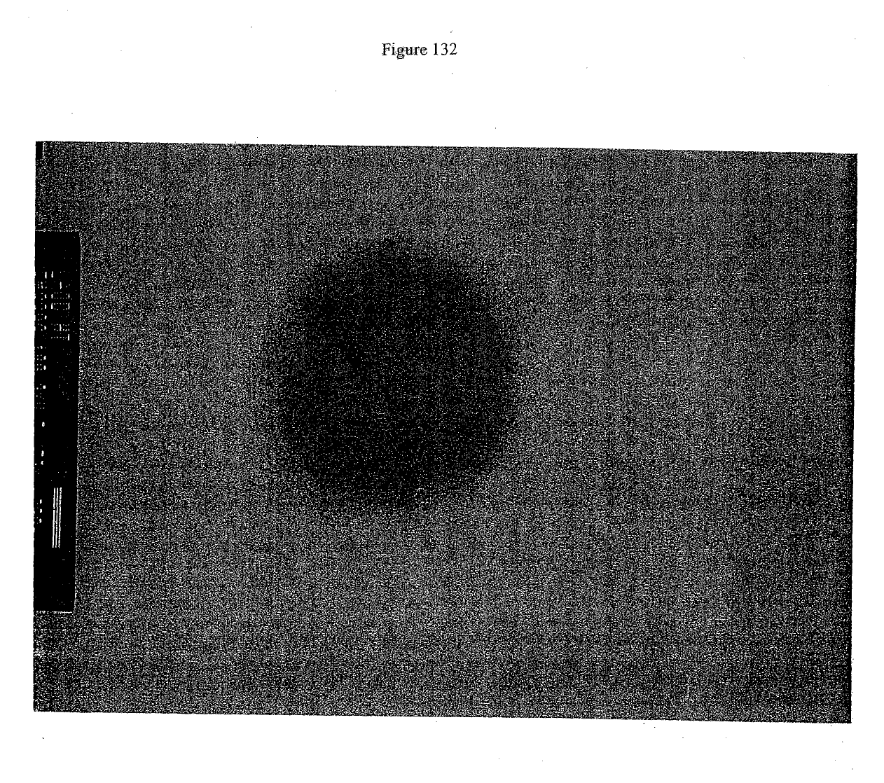

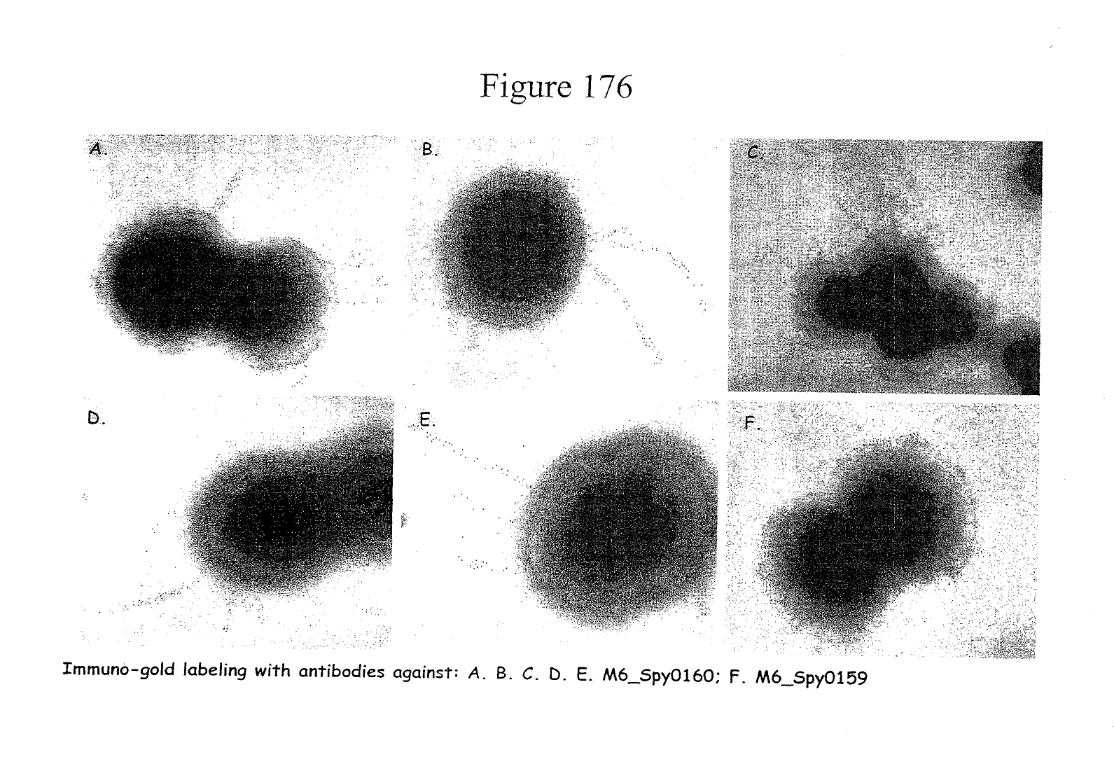

M6_Spy0160 appears to be present on the surface of GAS as part of oligomeric (pilus) structures. FIGS. 127-132 present electron micrographs of GAS serotype M6 strain 3650 immunogold stained for M6_Spy0160 using anti-M6_Spy0160 antiserum. Oligomeric or hyperoligomeric structures labelled with gold particles can be seen extending from the surface of the GAS in each of these FIGS., indicating the presence of multiple M6_Spy0160 polypeptides in the oligomeric or hyperoligomeric structures. FIG. 176A-F present electron micrographs of GAS M6 strain 2724 immunogold stained for M6_Spy0160 using anti-M6_Spy0160 antiserum (FIGS. 176A-E) or immunogold stained for M6_Spy0159 using anti-M6_Spy0159 antiserum (FIG. 176F). Oligomeric or hyperoligomeric structures labelled with gold particles can again be seen extending from the surface of the M6 strain 2724 GAS bacteria immunogold stained for M6_Spy0160. M6_Spy0159 is also detected on the surface of the M6 strain 2724 GAS.

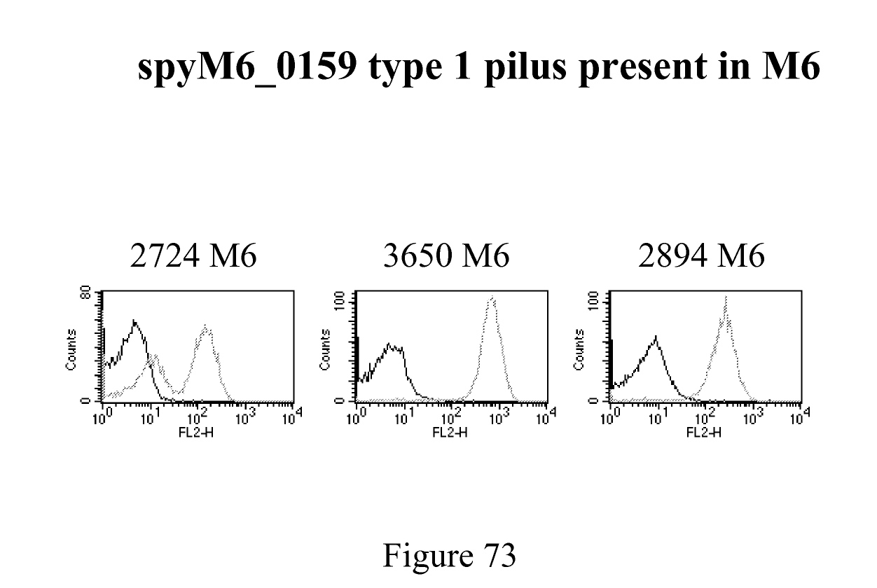

FACS analysis has confirmed that the GAS AI-1 surface proteins spyM6_{—}0159 and spyM6_{—}0160 are indeed expressed on the surface of GAS. FIG. 73 provides the results of FACS analysis for surface expression of spyM6_{—}0159 on each of GAS serotypes M6 2724, M6 3650, and M6 2894. A shift in fluorescence is observed for each GAS serotype when anti-spyM6_{—}0159 antiserum is present, demonstrating cell surface expression. Table 18, below, quantitatively summarizes the FACS fluorescence values obtained for each GAS serotype in the presence of pre-immune antiserum, anti-spyM6_{—}0159 antiserum, and the difference in fluorescence value between the pre-immune and anti-spyM6_{—}0159 antiserum.

TABLE 18
Summary of FACS values for surface expression of spyM6_0159

| 2724 | | | 3650 | | | 2894 | | |
|---|---|---|---|---|---|---|---|---|
| Pre- | Anti- | | Pre- | Anti- | | Pre- | Anti- | |
| immune | spyM6_0159 | Change | immune | spyM6_0159 | Change | immune | spyM6_0159 | Change |
| 134.84 | 427.48 | 293 | 149.68 | 712.62 | 563 | 193.86 | 597.8 | 404 |

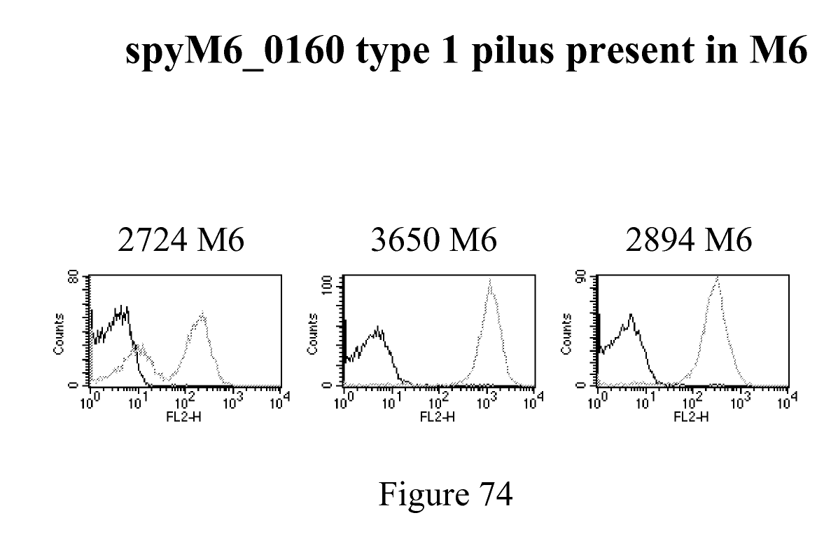

FIG. 74 provides the results of FACS analysis for surface expression of spyM6_{—}0160 on each of GAS serotypes M6 2724, M6 3650, and M6 2894. In the presence of anti-spyM6_{—}0160 antiserum, a shift in fluorescence is observed for each GAS serotype, which demonstrates its cell surface expression. Table 19, below, quantitatively summarizes the FACS fluorescence values obtained for each GAS serotype in the presence of pre-immune antiserum, anti-spyM6_{—}0160 antiserum, and the change in fluorescence value between the pre-immune and anti-spyM6_{—}0160 antiserum.

TABLE 19
Summary of FACS values for surface expression of spyM6_0160

| 2724 | | | 3650 | | | 2894 | | |
|---|---|---|---|---|---|---|---|---|
| Pre- | Anti- | | Pre- | Anti- | | Pre- | Anti- | |
| immune | spyM6_0160 | change | immune | spyM6_0160 | change | immune | spyM6_0160 | change |
| 117.12 | 443.24 | 326 | 128.57 | 776.39 | 648 | 125.87 | 621.17 | 495 |

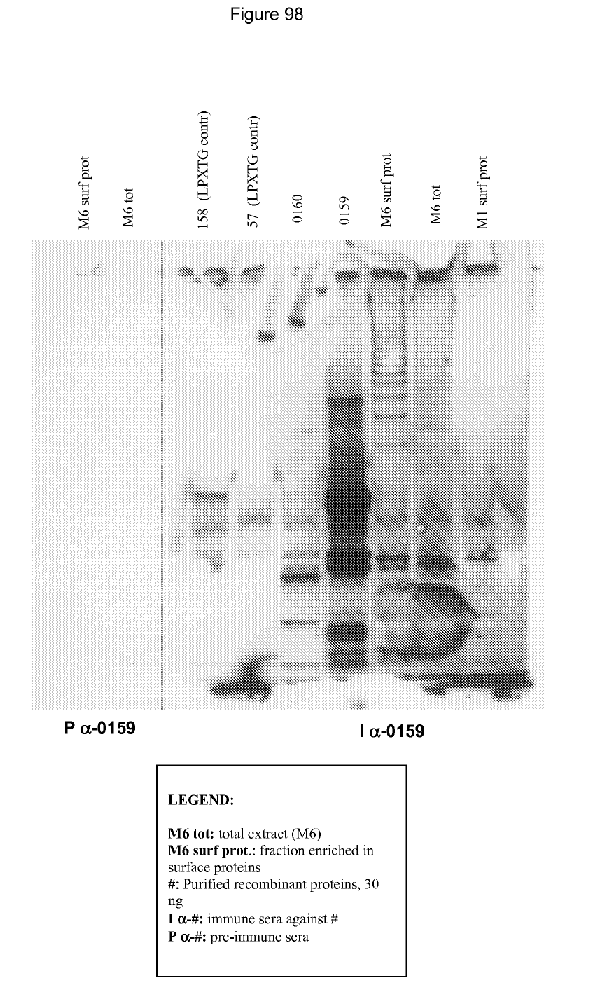

Surface expression of M6_Spy0159 and M6_Spy0160 on M6 serotype GAS has also been confirmed by Western blot analysis. FIG. 98 shows that while pre-immune sera (P α-0159) does not detect expression of M6_Spy0159 in GAS serotype M6, anti-M6_Spy0159 immune sera (I α-0159) is able to detect M6_Spy0159 protein in both total GAS M6 extracts (M6 tot) and GAS M6 fractions enriched for cell surface proteins (M6 surf prot). The M6_Spy0159 proteins detected in the total GAS M6 extracts or the GAS M6 extracts enriched for surface proteins are also present as high molecular weight structures, indicating that M6_Spy0159 may be in an oligomeric (pilus) form.

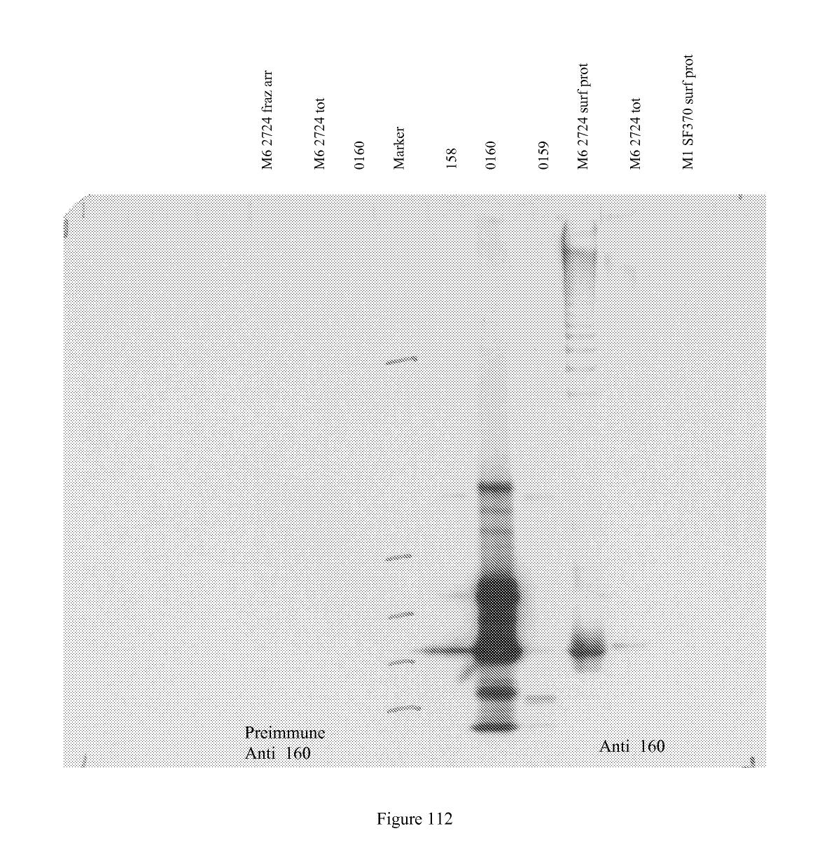

FIG. 112 shows that while preimmune sera (Preimmune Anti 106) does not detect expression of M6_Spy0160 in GAS serotype M6 strain 2724, anti-M6_Spy0160 immune sera (Anti 160) does in both total GAS M6 strain 2724 extracts (M6 2724 tot) and GAS M6 strain 2724 fractions enriched for surface proteins. The M6_Spy0160 proteins detected in the total GAS M6 strain 2724 extracts or the GAS M6 strain 2724 extracts enriched for surface proteins are also present as high molecular weight structures, indicating that M6_Spy0160 may be in an oligomeric (pilus) form.

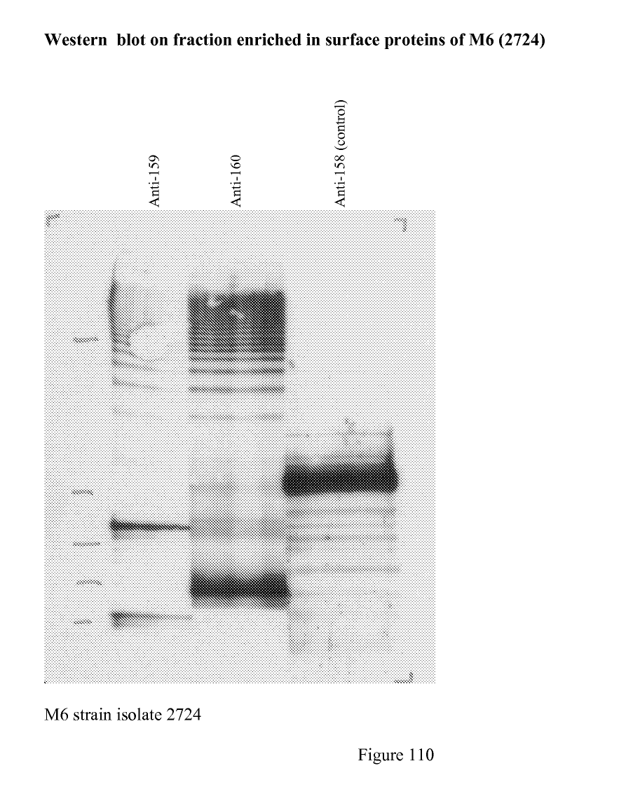

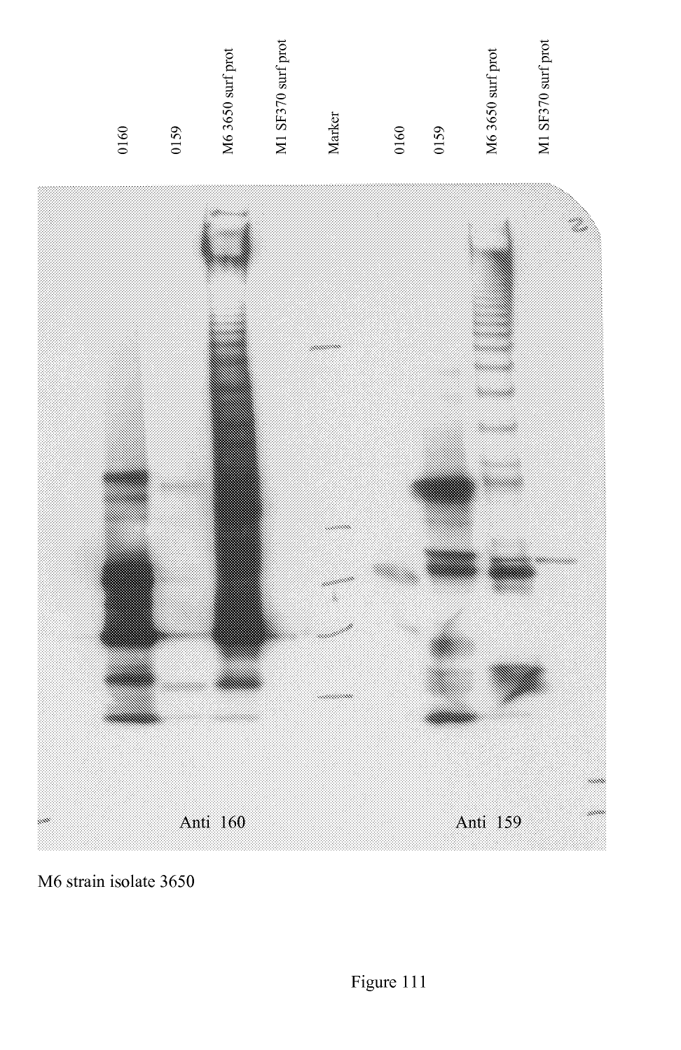

FIGS. 110 and 111 both further verify the presence of M6_Spy0159 and M6_Spy0160 in higher molecular weight structures on the surface of GAS. FIG. 110 provides a Western blot performed to detect M6_Spy0159 and M6_Spy0160 in GAS M6 strain 2724 extracts enriched for surface proteins. Antiserum raised against either M6_Spy0159 (Anti-159) or M6_Spy0160 (Anti-160) cross-hybridizes with high molecular weight structures (pili) in these extracts. FIG. 111 provides a similar Western blot that verifies the presence of M6_Spy0159 and M6_Spy0160 in high molecular weight structures in GAS M6 strain 3650 extracts enriched for surface proteins.

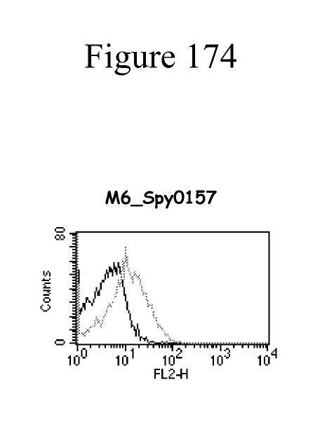

SpyM6_{—}0157 (a fibronectin-binding protein) may also be expressed on the surface of GAS serotype M6 bacteria. FIG. 174 shows the results of FACS analysis for surface expression of spyM6_{—}0157 on M6 strain 3650. A slight shift in fluorescence is observed, which demonstrates that some spyM6_{—}0157 may be expressed on the GAS cell surface.

### Adhesin Island Sequence Within M6: GAS Adhesin Island 2 (“GAS AI-2”)

A GAS Adhesin Island within M1 serotype (SF370) is outlined in Table 5 below. This GAS adhesin island 2 (“GAS AI-2”) comprises surface proteins, a SrtB sortase, a SrtC1 sortase and a RofA divergently transcribed transcriptional regulator.

GAS AI-2 surface proteins include GAS 15 (Cpa), Spy0128 (thought to be a fimbrial protein) and Spy0130 (a hypothetical protein). Preferably, each of these GAS AI-2 surface proteins includes an LPXTG sortase substrate motif, such as LPXTG (SEQ ID NO:122), VVXTG (SEQ ID NO:135), or EVXTG (SEQ ID NO:136).

GAS AI-2 includes a srtB type sortase and a srtC1 sortase. As discussed above, GAS SrtB sortases may preferably anchor surface proteins with an LPSTG (SEQ ID NO:166) motif, particularly where the motif is followed by a serine. GAS SrtC1 sortase may preferentially anchor surface proteins with a V(P/V)PTG (SEQ ID NO:167) motif. GAS SrtC1 may be differentially regulated by RofA.

GAS AI-2 may also include a LepA putative signal peptidase I protein.

TABLE 5
GAS AI-2 sequence from M1 isolate (SF370)

| | Sortase | |
|---|---|---|
| AI-2 | substrate | |
| sequence | sequence or | |
| identifier | sortase type | functional description |
| SPy0124 | | rofA regulatory protein |
| GAS15(not annotated | VVXTG | cpa |
| in SF370) | | |
| SPy0127 | | LepA putative signal peptidase I |
| SPy0128 (GAS16) | EVXTG | hypothetical protein (fimbrial) |
| SPy0129 (GAS17) | srtC1 | sortase |
| SPy0130 (GAS18) | LPXTG | hypothetical protein |
| SPy0131 | | conserved hypothetical protein |
| SPy0133 | | conserved hypothetical protein |
| SPy0135 (GAS20) | srtB | sortase (putative fimbrial- |
| | | associated protein) |

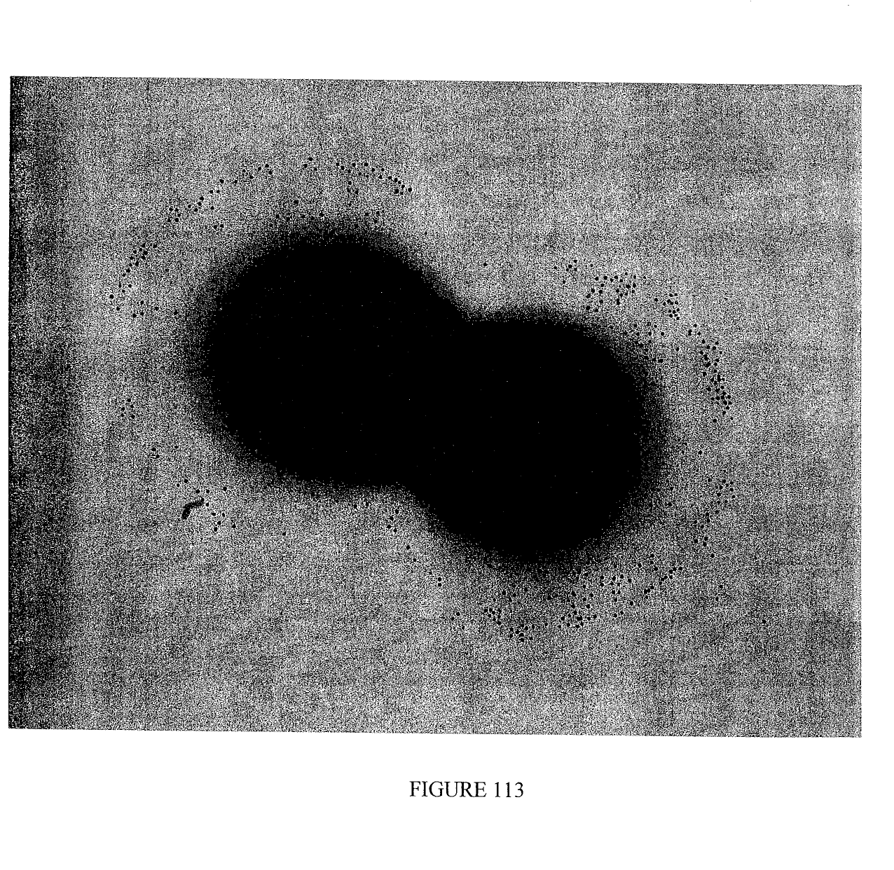

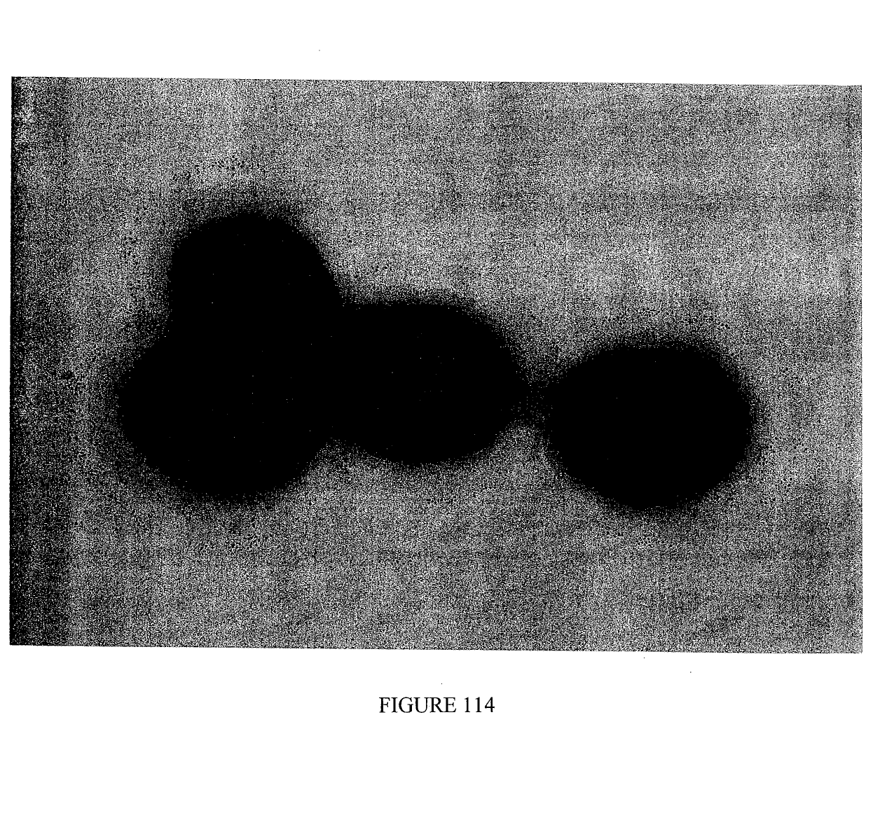

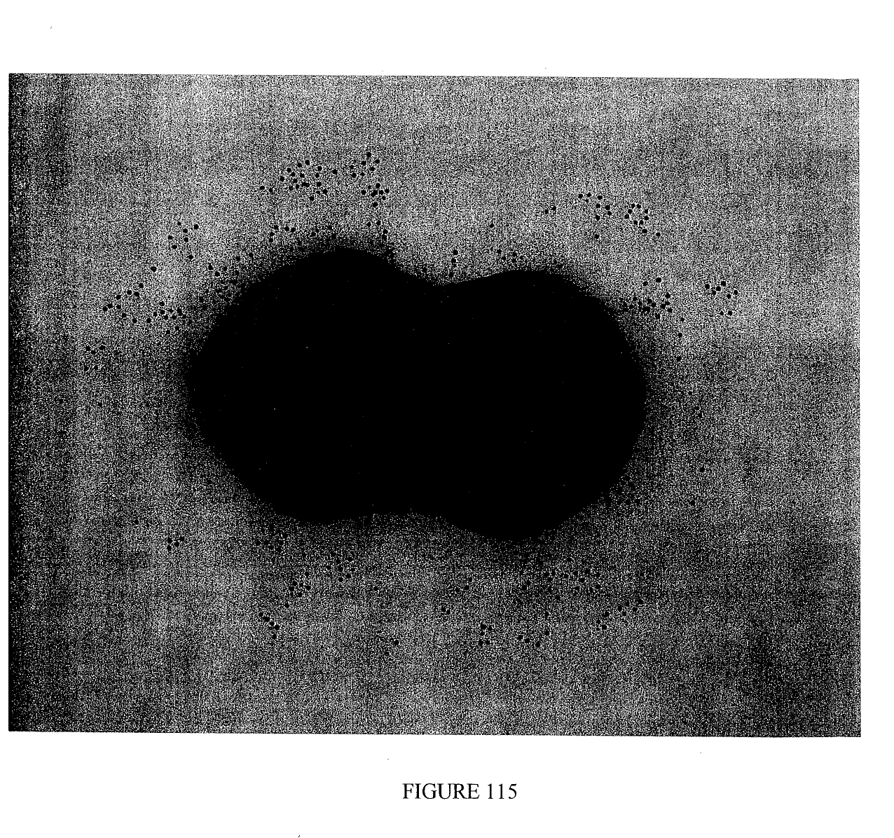

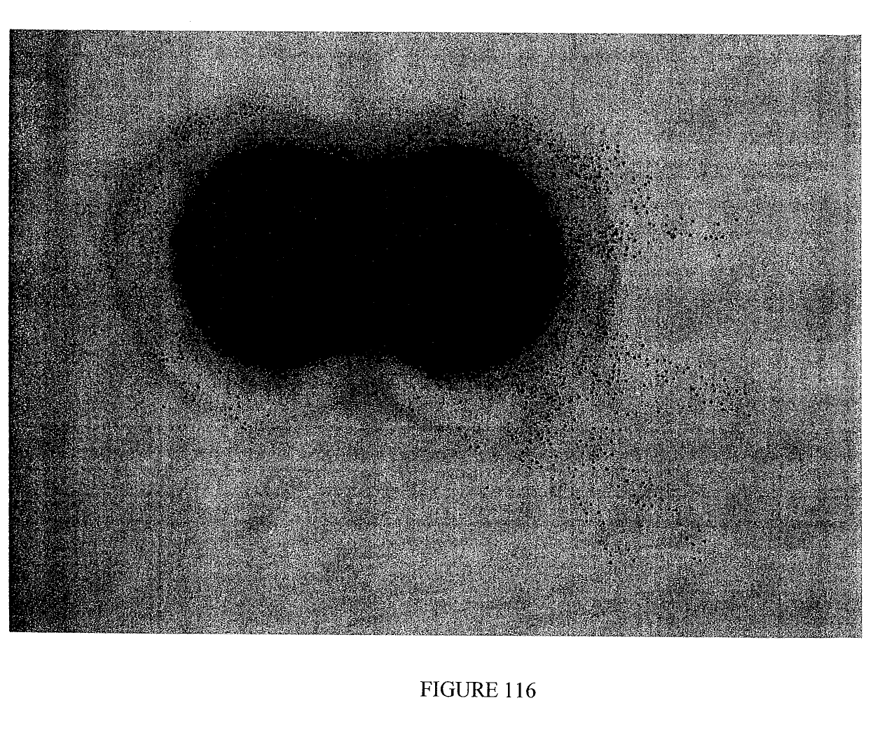

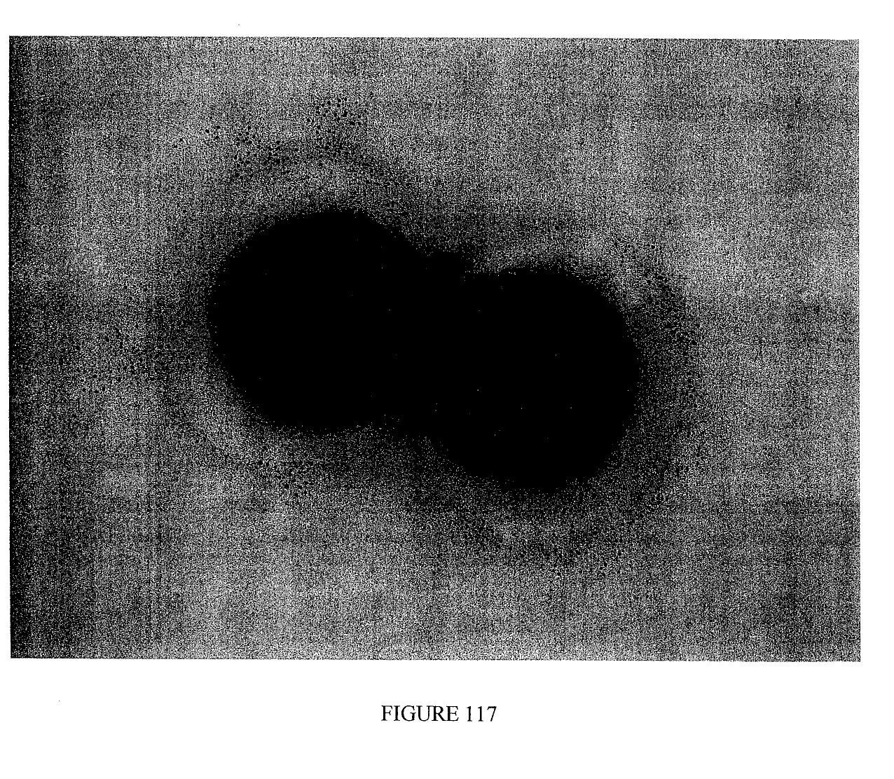

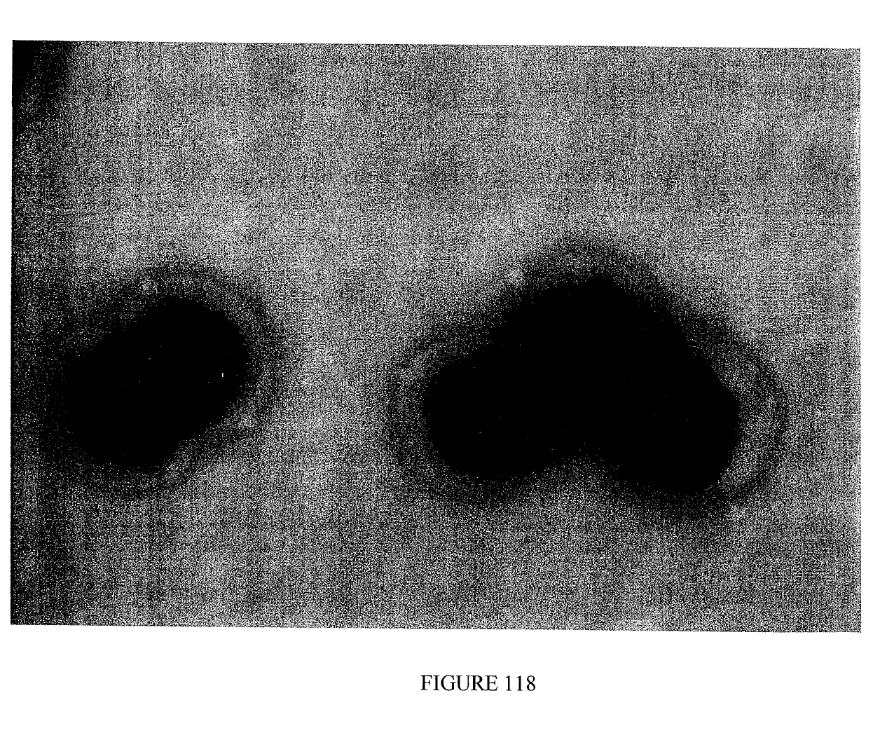

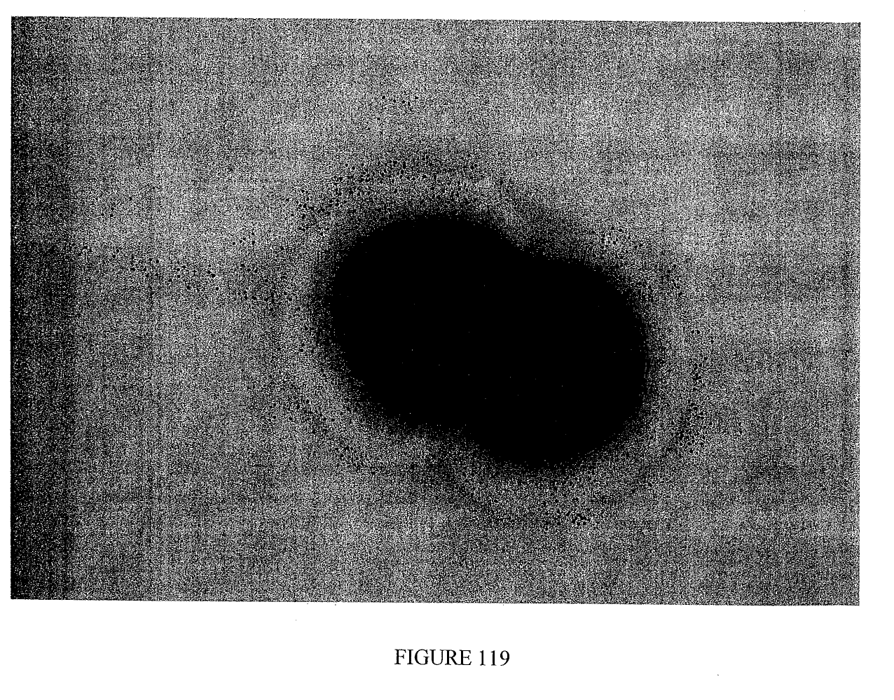

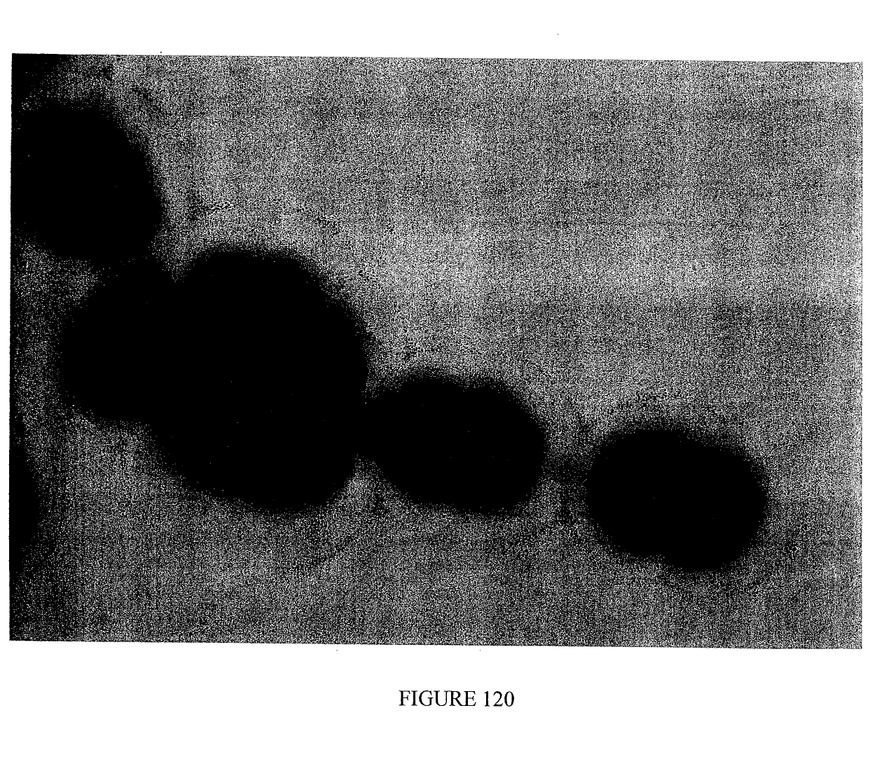

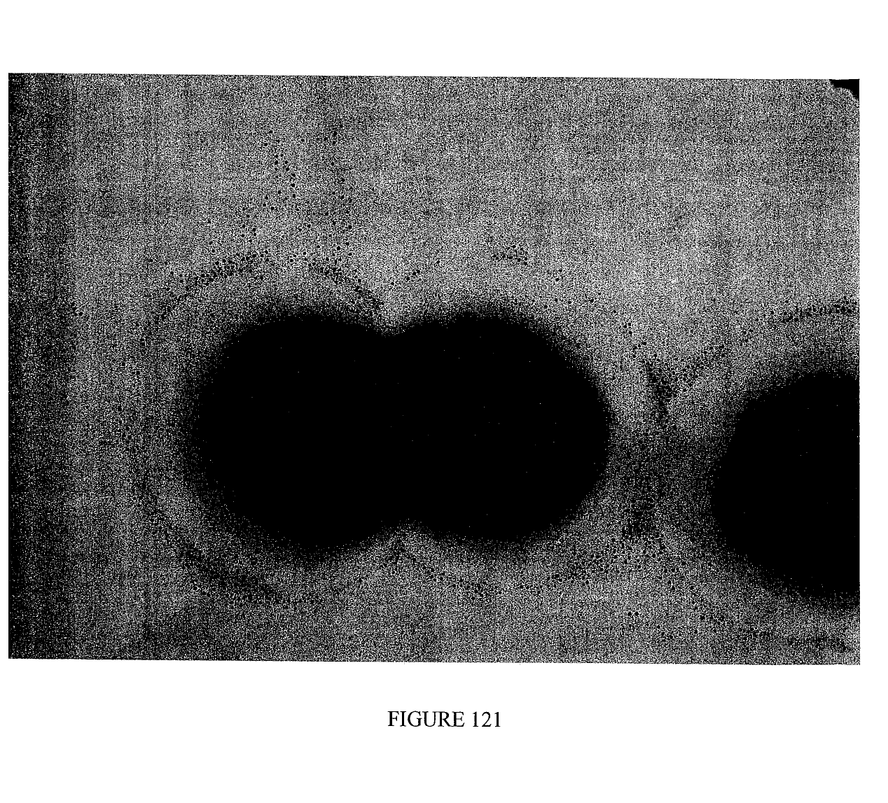

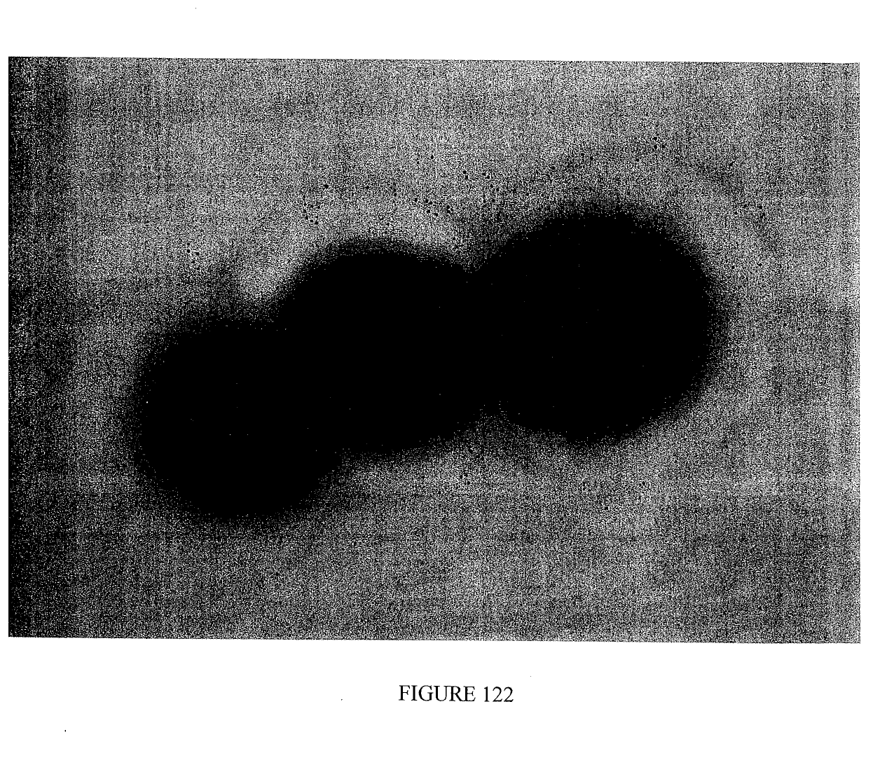

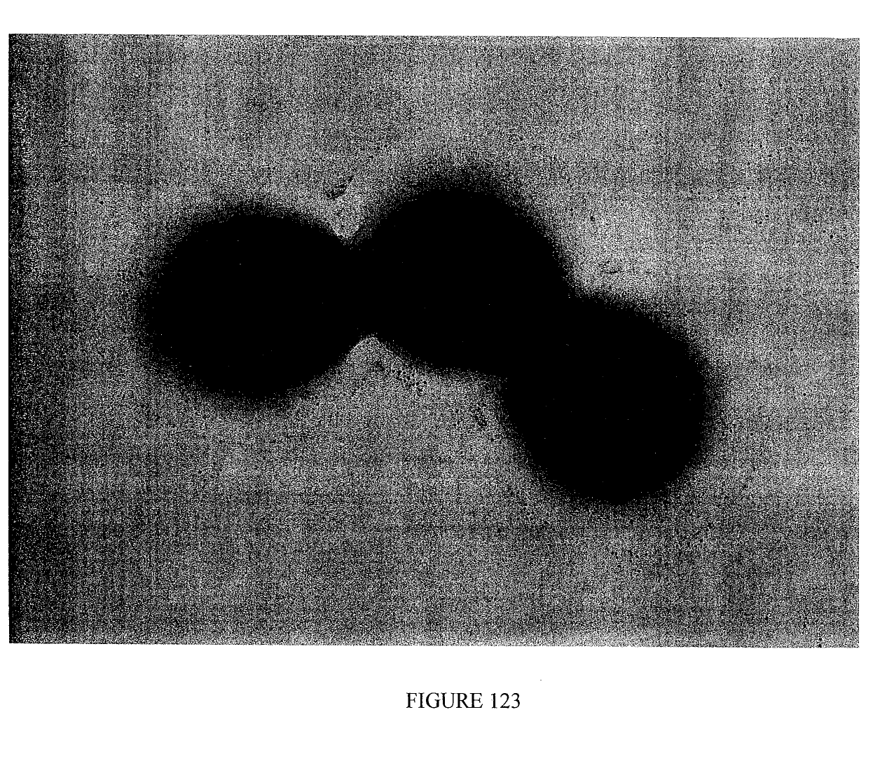

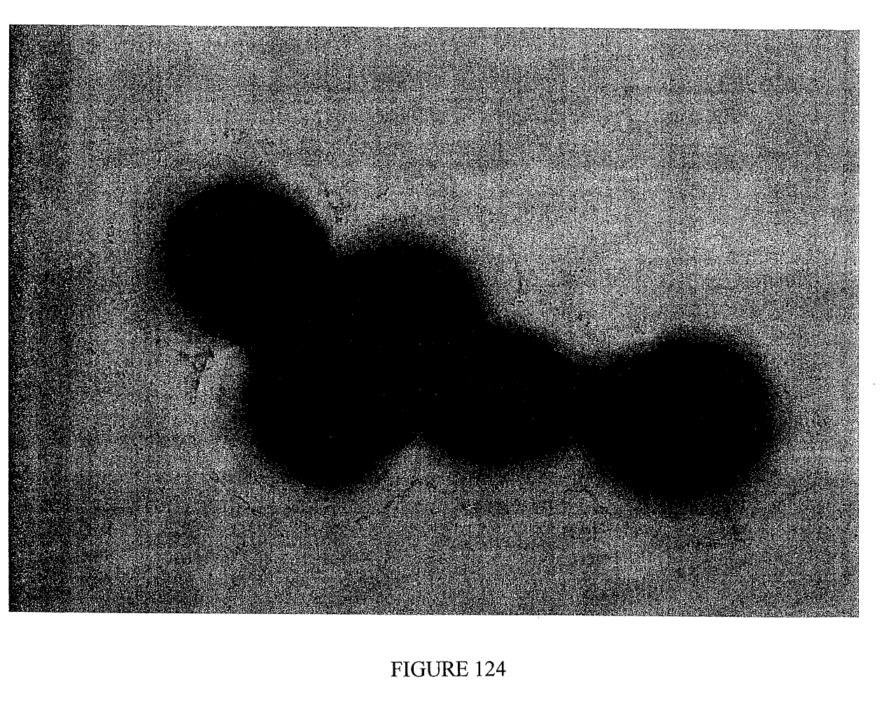

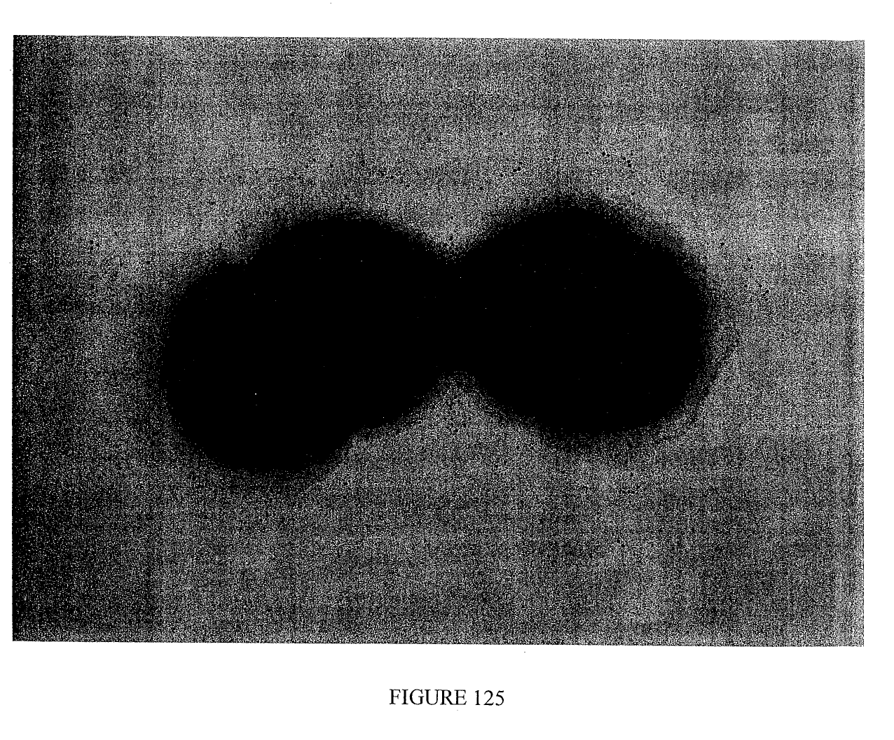

GAS 15, GAS 16, and GAS 18 appear to be present on the surface of GAS as part of oligomeric (pilus) structures. FIGS. 113-115 present electron micrographs of GAS serotype M1 strain SF370 immunogold stained for GAS 15 using anti-GAS 15 antiserum. FIGS. 116-121 provide electron micrographs of GAS serotype M1 strain SF370 immunogold stained for GAS 16 using anti-GAS 16 antiserum. FIGS. 122-125 present electron micrograph of GAS serotype M1 strain SF370 immunogold stained for GAS 18 using anti-GAS 18 antiserum. Oligomers of these proteins can be seen on the surface of SF370 bacteria in the immuno-gold stained micrographs.

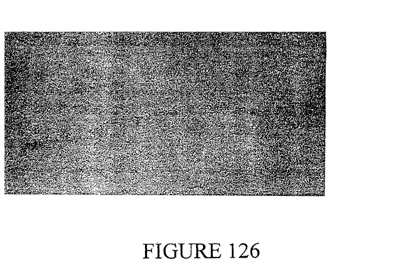

FIG. 126 reveals a hyperoligomer on the surface of a GAS serotype M1 strain SF370 bacterium immunogold stained for GAS 18. This long hyperoligomeric structure comprising GAS 18 stretches far out into the supernatant from the surface of the bacteria.

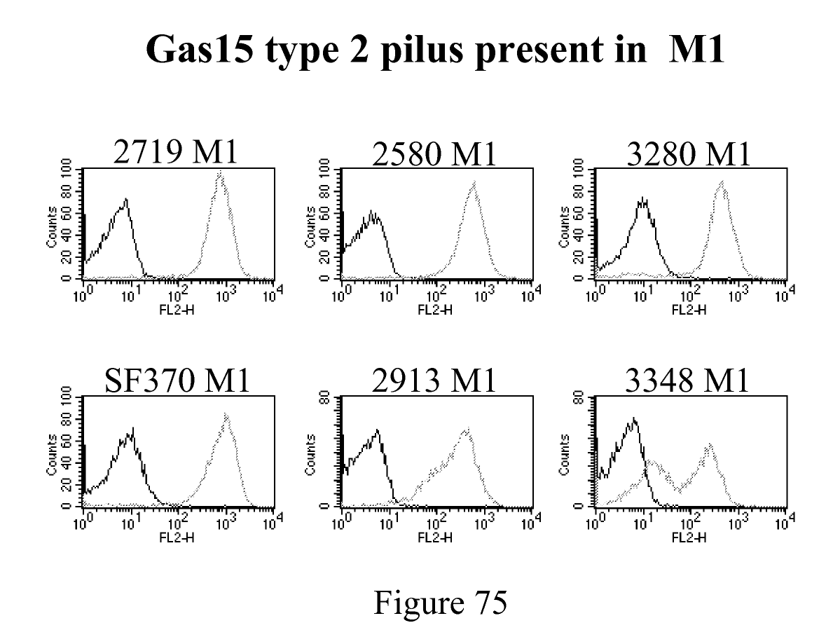

FACS analysis has confirmed that the GAS AI-2 surface proteins GAS 15, GAS 16, and GAS 18 are expressed on the surface of GAS. FIG. 75 provides the results of FACS analysis for surface expression of GAS 15 on each of GAS serotypes M1 2719, M1 2580, M1 3280, M1 SF370, M1 2913, and M1 3348. A shift in fluorescence is observed for each GAS serotype when anti-GAS 15 antiserum is present, demonstrating cell surface expression. Table 20, below, quantitatively summarizes the FACS fluorescence values obtained for each GAS serotype in the presence of pre-immune antiserum, anti-GAS 15 antiserum, and the difference in fluorescence value between the pre-immune and anti-GAS 15 antiserum.

TABLE 20
Summary of FACS values for surface expression of GAS 15

| Pre- | Anti-GAS | | Pre- | Anti-GAS | | Pre- | Anti-GAS | |
|---|---|---|---|---|---|---|---|---|
| immune | 15 | Change | immune | 15 | Change | immune | 15 | Change |
| 2719 | | | 2580 | | | 3280 | | |
| 159.46 | 712.71 | 553 | 123.9 | 682.84 | 559 | 217.02 | 639.69 | 423 |
| SF370 | | | 2913 | | | 3348 | | |
| 201.93 | 722.68 | 521 | 121.41 | 600.45 | 479 | 152.09 | 446.41 | 294 |

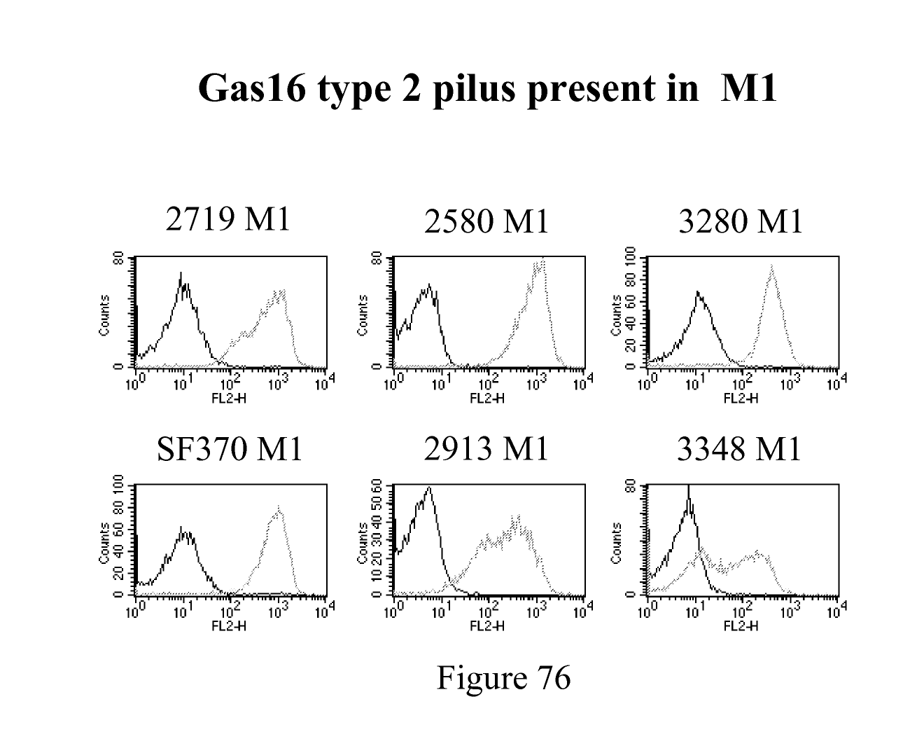

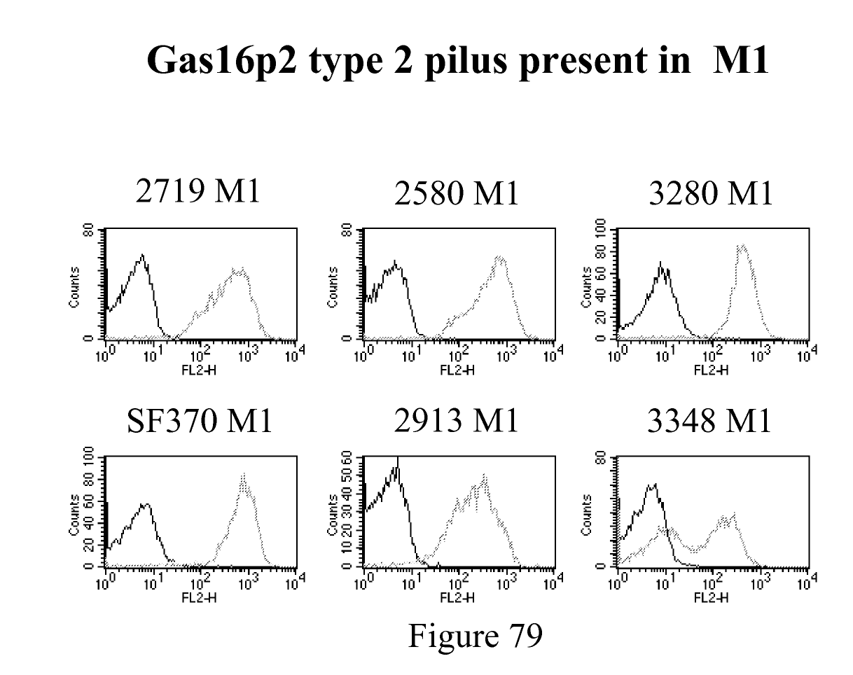

FIGS. 76 and 79 provide the results of FACS analysis for surface expression of GAS 16 on each of GAS serotypes M1 2719, M1 2580, M1 3280, M1 SF370, M1 2913, and M1 3348. The FACS data in FIG. 76 was obtained using antisera was raised against full length GAS 16. In the presence of this anti-GAS 16 antiserum, a shift in fluorescence is observed for each GAS serotype, demonstrating its cell surface expression. Table 21, below, quantitatively summarizes the FACS fluorescence values obtained for each GAS serotype in the presence of pre-immune antiserum, anti-GAS 16 antiserum, and the change in fluorescence value between the pre-immune and anti-GAS 16 antiserum.

TABLE 21
Summary of FACS values for surface expression of GAS 16

| Pre- | Anti-GAS | | Pre- | Anti-GAS | | Pre- | Anti-GAS | |
|---|---|---|---|---|---|---|---|---|
| immune | 16 | Change | immune | 16 | Change | immune | 16 | Change |
| 2719 | | | 2580 | | | 3280 | | |
| 233.27 | 690.09 | 457 | 133.82 | 732.29 | 598 | 264.47 | 649.43 | 385 |
| SF370 | | | 2913 | | | 3348 | | |
| 237.2 | 727.46 | 490 | 138.52 | 588.04 | 450 | 180.56 | 420.93 | 240 |

The FACS data in FIG. 79 was obtained using antisera was raised against a truncated GAS 16, which is encoded by SEQ ID NO:179, shown below.

SEQ ID NO: 179:
GCTACAACAGTTCACGGGGAGACTGTTGTAAACGGAGCCAAACTAACAGT
TACAAAAAACCTTGATTTAGTTAATAGCAATGCATTAATTCCAAATACAG
ATTTTACATTTAAAATCGAACCTGATACTACTGTCAACGAAGACGGAAAT
AAGTTTAAAGGTGTAGCTTTGAACACACCGATGACTAAAGTCACTTACAC
CAATTCAGATAAAGGTGGATCAAATACGAAAACTGCAGAATTTGATTTTT
CAGAAGTTACTTTTGAAAAACCAGGTGTTTATTATTACAAAGTAACTGAG
GAGAAGATAGATAAAGTTCCTGGTGTTTCTTATGATACAACATCTTACAC
TGTTCAAGTTCATGTCTTGTGGAATGAAGAGCAACAAAAACCAGTAGCTA
CTTATATTGTTGGTTATAAAGAAGGTAGTAAGGTGCCAATTCAGTTCAAA
AATAGCTTAGATTCTACTACATTAACGGTGAAGAAAAAAGTTTCAGGTAC
CGGTGGAGATCGCTCTAAAGATTTTAATTTTGGTCTGACTTTAAAAGCAA
ATCAGTATTATAAGGCGTCAGAAAAAGTCATGATTGAGAAGACAACTAAA
GGTGGTCAAGCTCCTGTTCAAACAGAGGCTAGTATAGATCAACTCTATCA
TTTTACCTTGAAAGATGGTGAATCAATCAAAGTCACAAATCTTCCAGTAG
GTGTGGATTATGTTGTCACTGAAGACGATTACAAATCAGAAAAATATACA
ACCAACGTGGAAGTTAGTCCTCAAGATGGAGCTGTAAAAAATATCGCAGG
TAATTCAACTGAACAAGAGACATCTACTGATAAAGATATGACCATTACTT
TTACAAATAAAAAAGATTT

In the presence of this anti-GAS 16 antiserum, a shift in fluorescence is observed for each GAS serotype, demonstrating its cell surface expression. Table 22, below, quantitatively summarizes the FACS fluorescence values obtained for each GAS serotype in the presence of pre-immune antiserum, anti-GAS 16 antiserum, and the change in fluorescence value between the pre-immune and anti-GAS 16 antiserum.

TABLE 22
Summary of FACS values for surface expression of GAS 16 using a second antisera

| Pre- | Anti-GAS | | Pre- | Anti-GAS | | Pre- | Anti-GAS | |
|---|---|---|---|---|---|---|---|---|
| immune | 16 | Change | immune | 16 | Change | immune | 16 | Change |
| 2719 | | | 2580 | | | 3280 | | |
| 141.55 | 650.22 | 509 | 119.57 | 672.35 | 553 | 209.18 | 666.71 | 458 |
| SF370 | | | 2913 | | | 3348 | | |
| 159.92 | 719.32 | 559 | 115.97 | 585.9 | 470 | 146.1 | 414.01 | 268 |

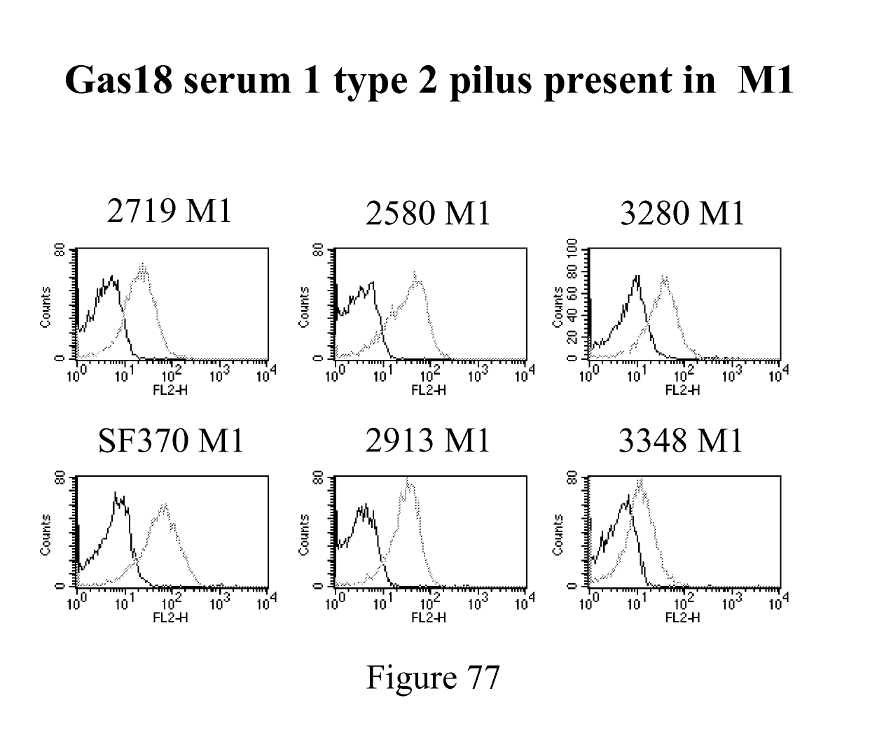

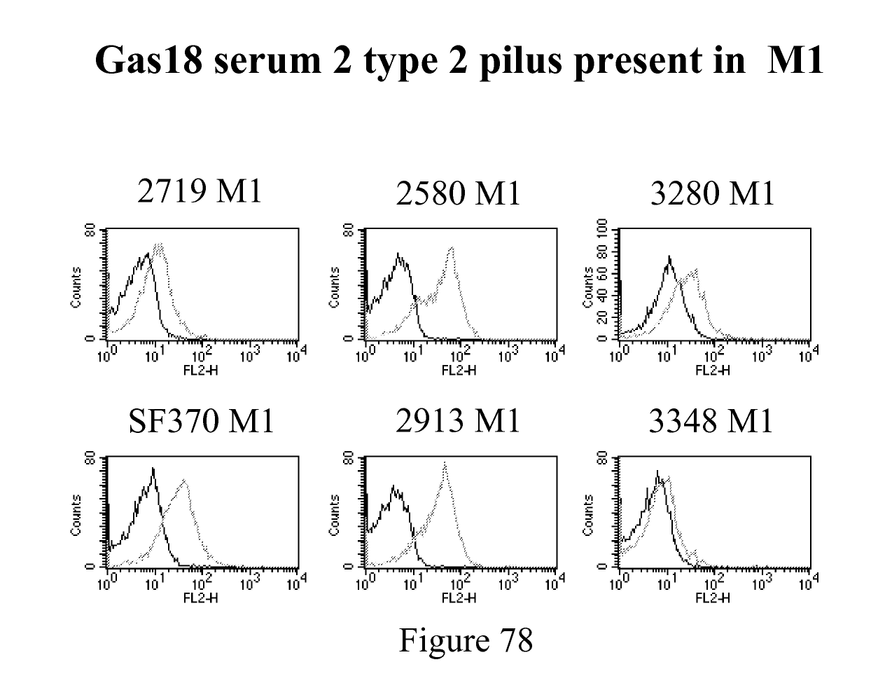

FIGS. 77 and 78 provide the results of FACS analysis for surface expression of GAS 18 on each of GAS serotypes M1 2719, M1 2580, M1 3280, M1 SF370, M1 2913, and M1 3348. The antiserum used to obtain the FACS data in each of FIGS. 77 and 78 was different, although each was raised against full length GAS 18. In the presence of each of the anti-GAS 18 antisera, a shift in fluorescence is observed for each GAS serotype, demonstrating its cell surface expression. Tables 23 and 24, below, quantitatively summarizes the FACS fluorescence values obtained for each GAS serotype in the presence of pre-immune antiserum, first or second anti-GAS 18 antiserum, and the change in fluorescence value between the pre-immune and first or second anti-GAS 18 antiserum.

TABLE 23
Summary of FACS values for surface expression of GAS 18

| Pre- | Anti-GAS | | Pre- | Anti-GAS | | Pre- | Anti-GAS | |
|---|---|---|---|---|---|---|---|---|
| immune | 18 | Change | immune | 18 | Change | immune | 18 | Change |
| 2719 | | | 2580 | | | 3280 | | |
| 135.68 | 327.98 | 192 | 116.32 | 379.41 | 263 | 208.12 | 380.84 | 173 |
| SF370 | | | 2913 | | | 3348 | | |
| 185.39 | 438.23 | 253 | 119.95 | 373.32 | 253 | 147.12 | 266.51 | 119 |

TABLE 24
Summary of FACS values for surface expression of GAS 18 using a second antisera

| Pre- | Anti-GAS | | Pre- | Anti-GAS | | Pre- | Anti-GAS | |
|---|---|---|---|---|---|---|---|---|
| immune | 18 | Change | immune | 18 | Change | immune | 18 | Change |
| 2719 | | | 2580 | | | 3280 | | |
| 150.4 | 250.39 | 100 | 139.18 | 386.38 | 247 | 253.38 | 347.72 | 94 |
| SF370 | | | 2913 | | | 3348 | | |
| 188.64 | 373.11 | 184 | 124.94 | 384.82 | 260 | 168.8 | 213.65 | 45 |

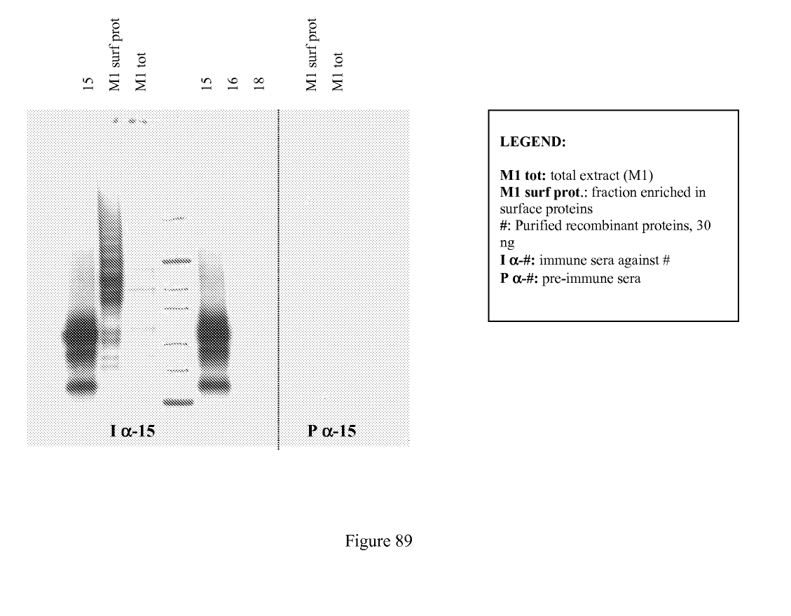

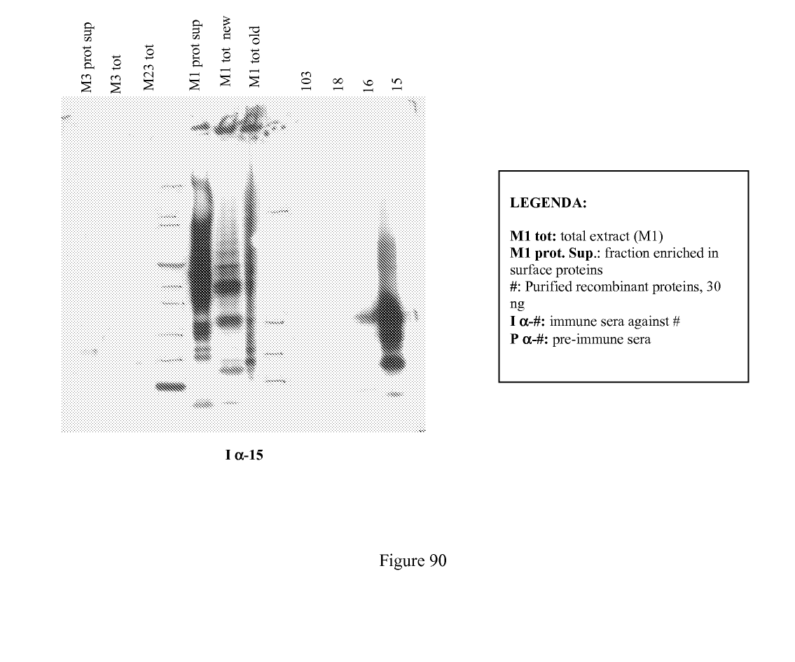

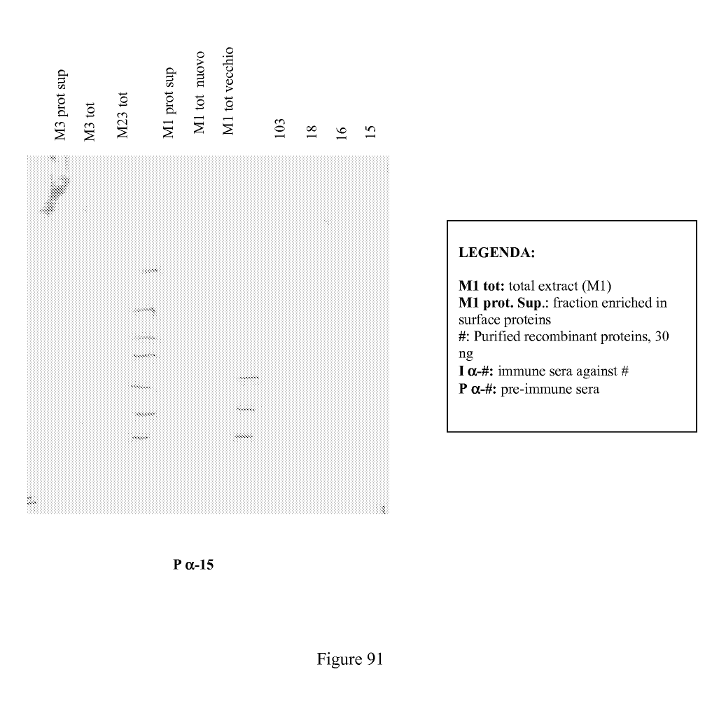

Surface expression of GAS 15, GAS 16, and GAS 18 on M1 serotype GAS has also been confirmed by Western blot analysis. FIG. 89 shows that while pre-immune sera does not detect GAS M1 expression of GAS 15, anti-GAS 15 immune sera is able to detect GAS 15 protein in both total GAS M1 extracts and GAS M1 proteins enriched for cell surface proteins. The GAS 15 proteins detected in the M1 extracts enriched for surface proteins are also present as high molecular weight structures, indicating that GAS 15 may be in an oligomeric (pilus) form. FIG. 90 also shows the results of Western blot analysis of M1 serotype GAS using anti-GAS 15 antisera. Again, the lanes that contain GAS M1 extracts enriched for surface proteins (M1 prot sup) show the presence of high molecular weight structures that may be oligomers of GAS 15. FIG. 91 provides an additional Western blot identical to that of FIG. 90, but that was probed with pre-immune sera. As expected, no proteins were detected on this membrane.

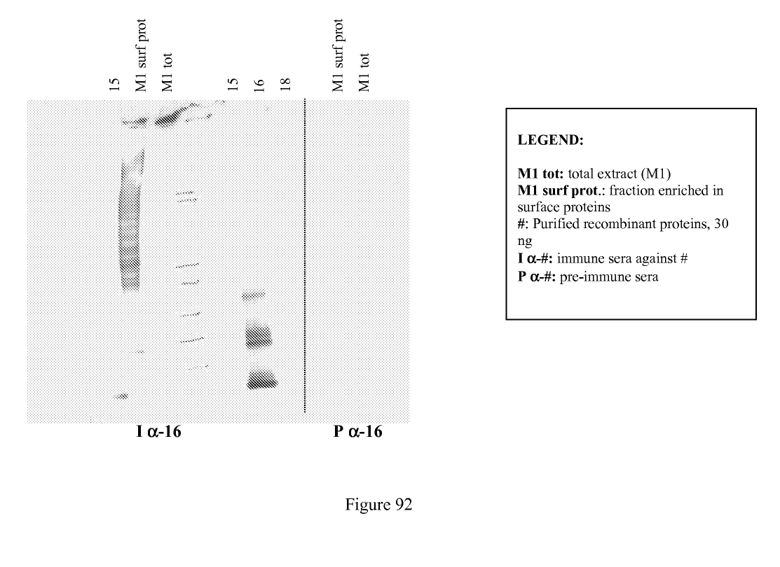

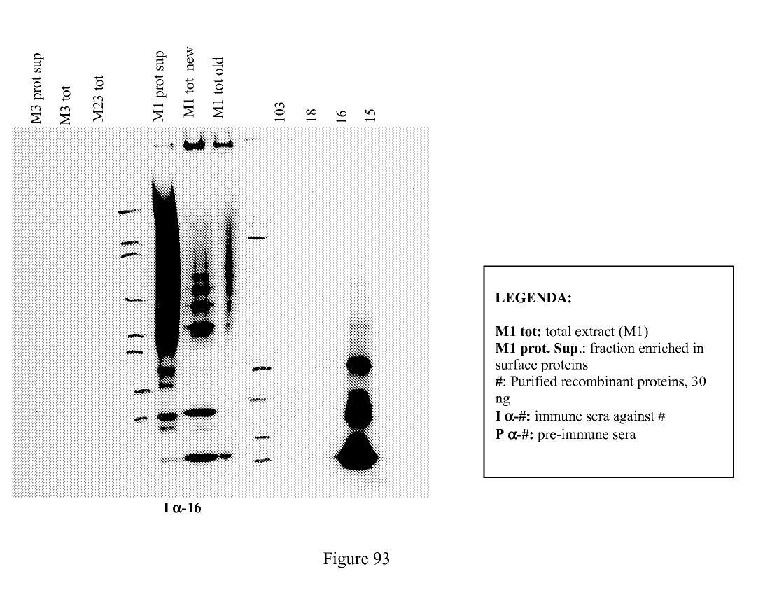

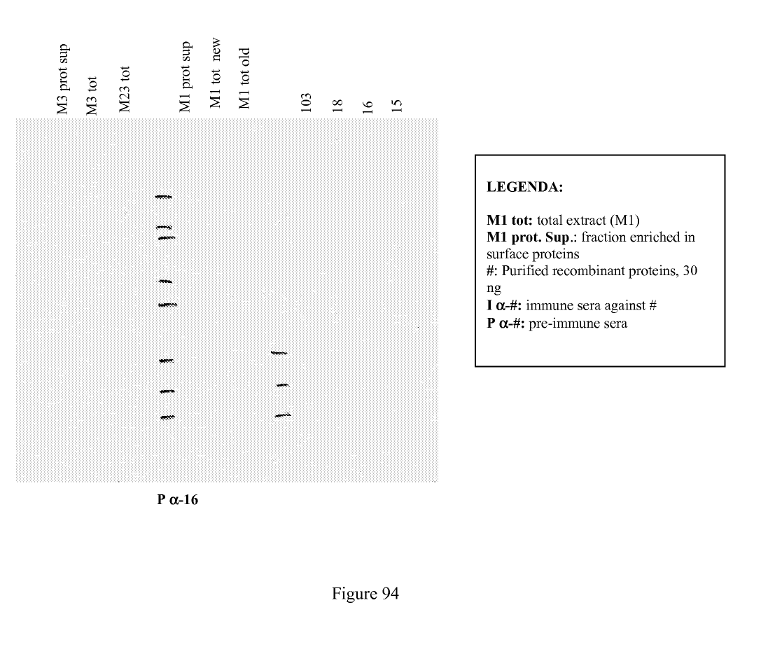

FIG. 92 provides a Western blot that was probed for GAS 16 protein. While pre-immune sera does not detect GAS M1 expression of GAS 16, anti-GAS 16 immune sera is able to detect GAS 16 protein in GAS M1 extracts enriched for cell surface proteins. The GAS 16 proteins detected in the M1 extracts enriched for surface proteins are present as high molecular weight structures, indicating that GAS 16 may be in an oligomeric (pilus) form. FIG. 93 also shows the results of Western blot analysis of M1 serotype GAS using anti-GAS 16 antisera. The lanes that contain total GAS M1 protein (M1 tot new and M1 tot old) and the lane that contains GAS M1 extracts enriched for surface proteins (M1 prot sup) show the presence of high molecular weight structures that may be oligomers of GAS 16. FIG. 94 provides an additional Western blot identical to that of FIG. 93, but that was probed with pre-immune sera. As expected, no proteins were detected on this membrane.

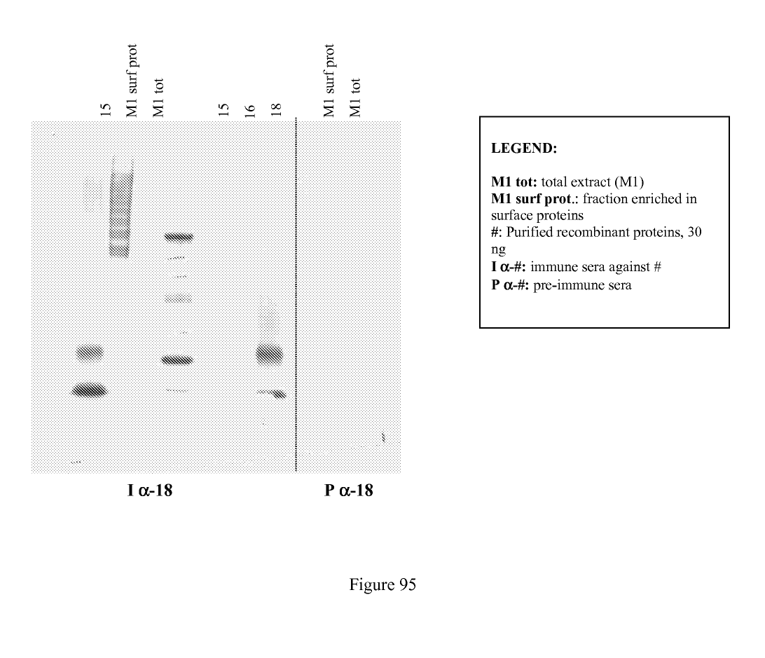

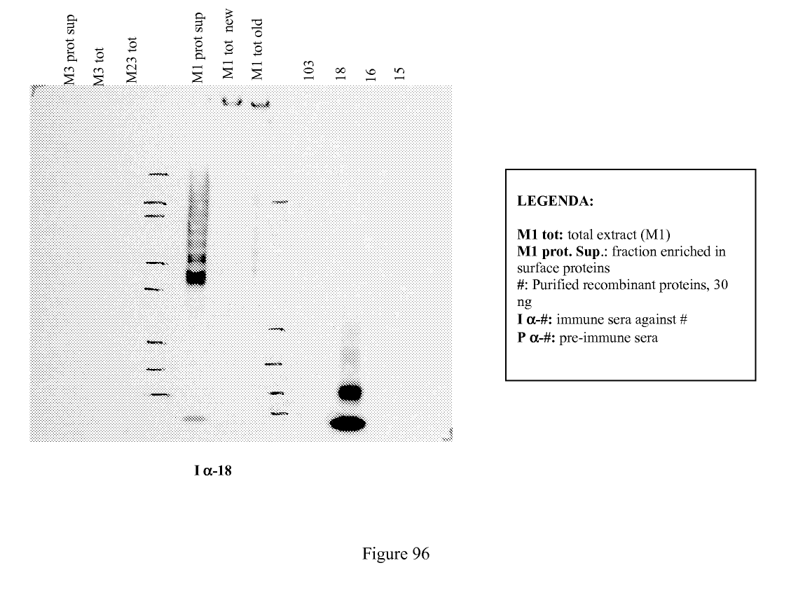

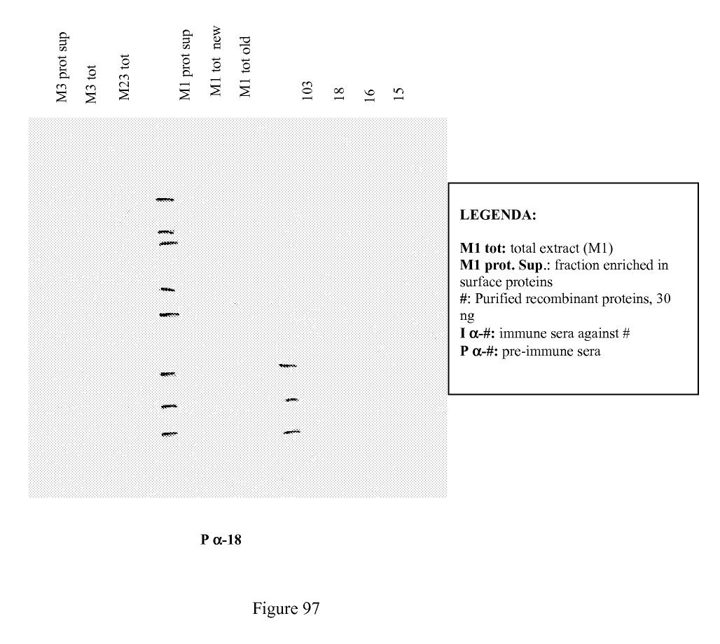

FIG. 95 provides a Western blot that was probed for GAS 18 protein. While pre-immune sera does not detect GAS M1 expression of GAS 18, anti-GAS 18 immune sera is able to detect GAS 18 protein in GAS M1 extracts enriched for cell surface proteins. The GAS 18 proteins detected in the M1 extracts enriched for surface proteins are present as high molecular weight structures, indicating that GAS 18 may be in an oligomeric (pilus) form. FIG. 96 also shows the results of Western blot analysis of M1 serotype GAS using anti-GAS 18 antisera. The lane that contains GAS M1 extracts enriched for surface proteins (M1 prot sup) show the presence of high molecular weight structures that may be oligomers of GAS 18. FIG. 97 provides an additional Western blot identical to that of FIG. 96, but that was probed with pre-immune sera. As expected, no proteins were detected on this membrane.

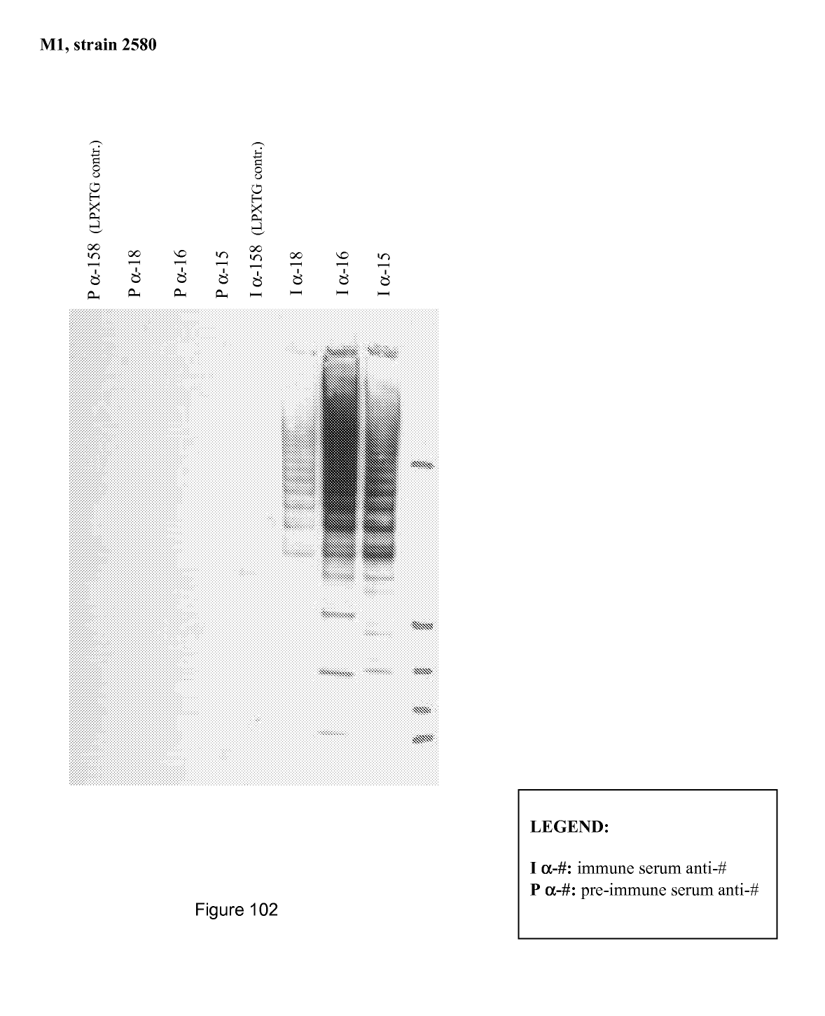

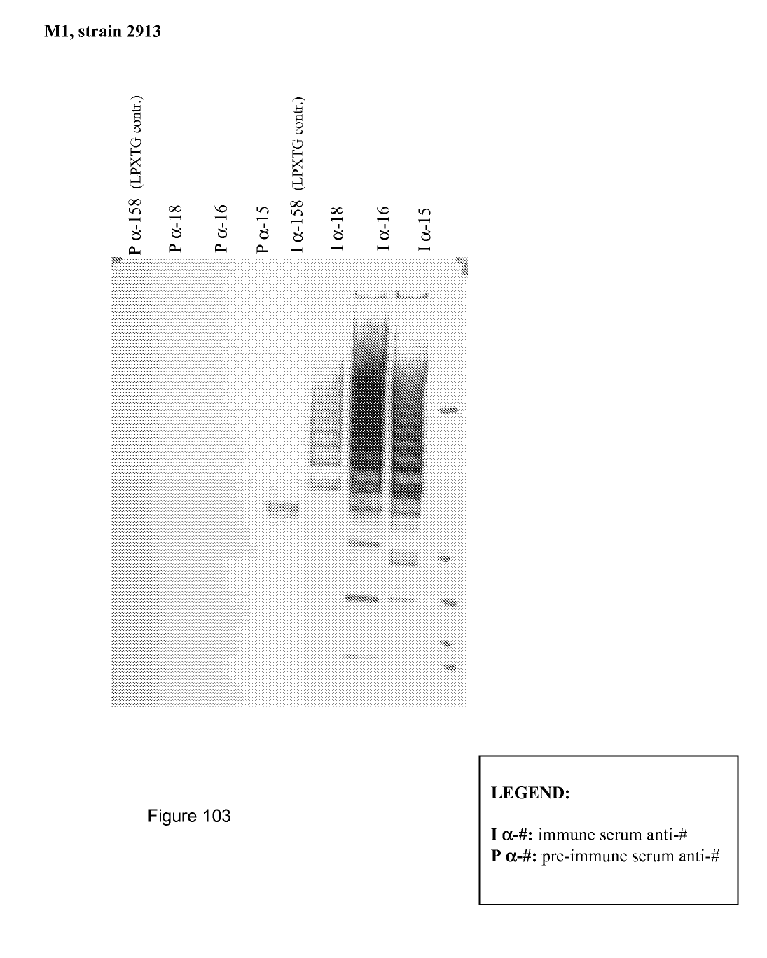

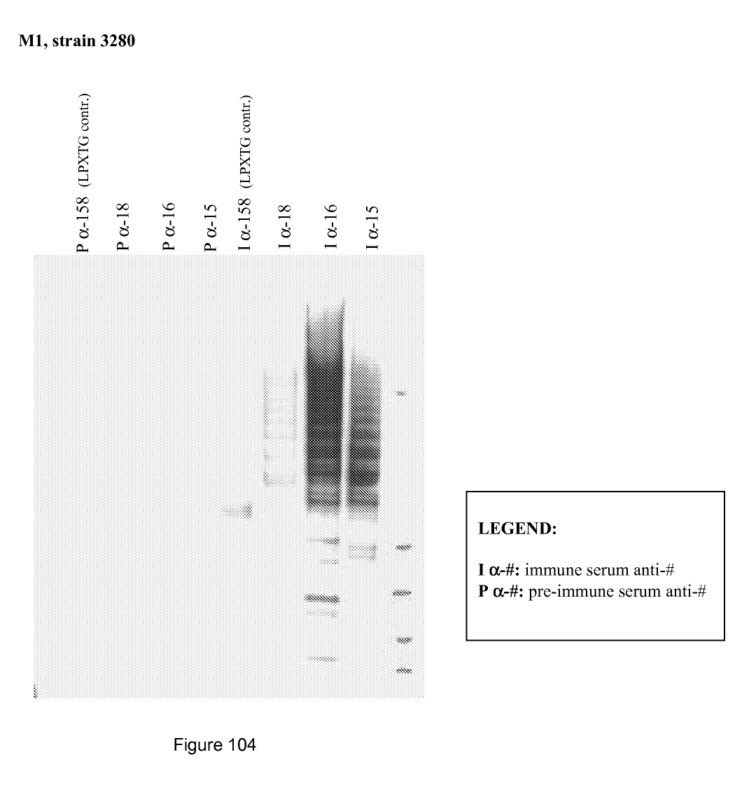

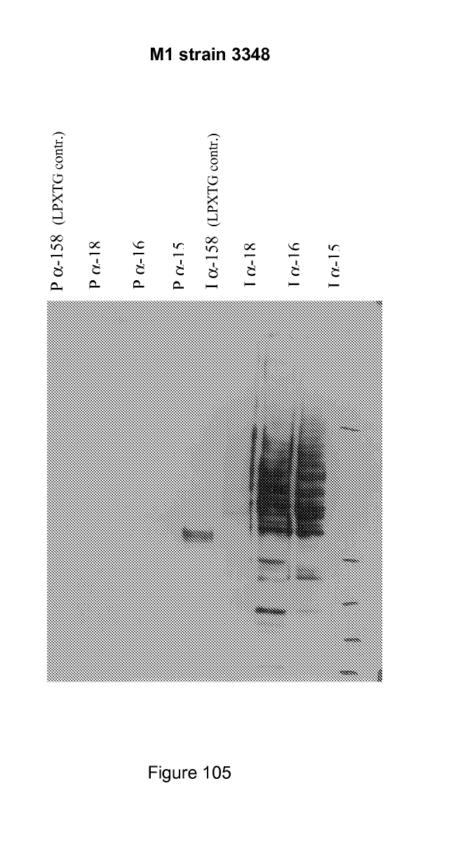

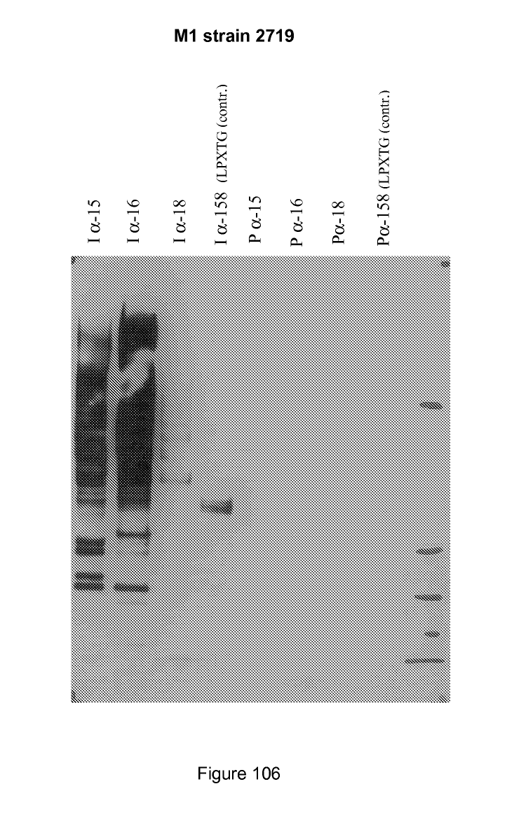

FIGS. 102-106 provide additional Western blots to verify the presence of GAS 15, GAS 16, and GAS 18 in high molecular weight structures in GAS. Each Western blot was performed using proteins from a different GAS M1 strain, 2580, 2913, 3280, 3348, and 2719. Each Western blot was probed with antisera raised against each of GAS 15, GAS 16, and GAS 18. As can be seen in FIGS. 102-106, none of the Western blots shows detection of proteins using pre-immune serum (Pα-158, Pα-15, Pα-16, or Pα-18), while each Western blot shows cross-hybridization of the GAS 15 (Iα-15), GAS 16 (Iα-16), and GAS 18 (Iα-18) antisera to high molecular weight structures. Thus, these Western blots confirm that GAS 15, GAS 16, and GAS 18 can be present in pili in GAS M1.

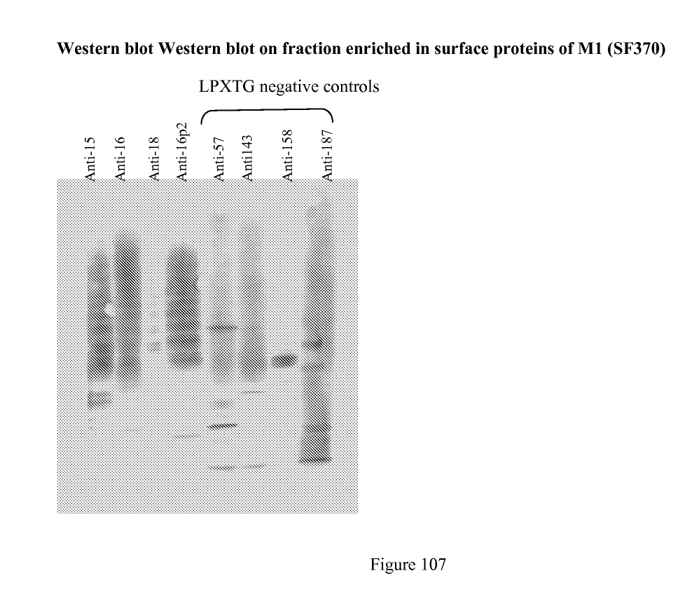

FIG. 107 provides a similar Western blot performed to detect GAS 15, GAS 16, and GAS 18 proteins in a GAS serotype M1 strain SF370 protein fraction enriched for surface proteins. This Western blot also shows detection of GAS 15 (Anti-15), GAS 16 (Anti-16), and GAS 18 (Anti-18) as high molecular weight structures.

(3) Adhesin Island Sequence Within M3, M5, and M18: GAS Adhesin Island 3 (“GAS AI-3”)

GAS Adhesin Island sequences within M3, M5, and M18 serotypes are outlined in Tables 6-8 and 10 below. This GAS adhesin island 3 (“GAS AI-3”) comprises surface proteins, a SrtC2 sortase, and a Negative transcriptional regulator (Nra) divergently transcribed transcriptional regulator.

GAS AI-3 surface proteins within include a collagen binding protein, a fimbrial protein, a F2 like fibronectin-binding protein. GAS AI-3 surface proteins may also include a hypothetical surface protein. Preferably, each of these GAS AI-3 surface proteins include an LPXTG sortase substrate motif, such as LPXTG (SEQ ID NO:122), VPXTG (SEQ ID NO:137), QVXTG (SEQ ID NO:138) or LPXAG (SEQ ID NO:139).

GAS AI-3 includes a SrtC2 type sortase. GAS SrtC2 type sortases may preferably anchor surface proteins with a QVPTG (SEQ ID NO:140) motif, particularly when the motif is followed by a hydrophobic region and a charged C terminus tail. GAS SrtC2 may be differentially regulated by Nra.

GAS AI-3 may also include a LepA putative signal peptidase I protein. GAS AI-3 may also include a putative multiple sugar metabolism regulator.

TABLE 6
GAS AI-3 sequences from M3 isolate (MGAS315)

| | Sortase | |
|---|---|---|
| AI-3 | substrate | |
| sequence | sequence or | |
| identifier | sortase type | Functional description |
| SpyM3_0097 | | Negative transcriptional regulator (Nra) |
| SpyM3_0098 | VPXTG | putative collagen binding protein (Cpb) |
| SpyM3_0099 | | LepA putative signal peptidase I |
| SpyM3_0100 | QVXTG | conserved hypothetical protein |
| | | (fimbrial) |
| SpyM3_0101 | SrtC2 | sortase |
| SpyM3_0102 | LPXAG | hypothetical protein |
| SpyM3_0103 | | putative multiple sugar metabolism |
| | | regulator |
| SpyM3_0104 | LPXTG | protein F2 like fibronectin-binding |
| | | protein |

TABLE 7
GAS AI-3 sequence from M3 isolate (SSI-1)

| | Sortase | |
|---|---|---|
| AI-3 | Substrate | |
| sequence | seq. or | |
| identifier | sortase type | Functional description |
| SPs0099 | | Negative transcriptional regulator (Nra) |
| SPs0100 | VPXTG | putative collagen binding protein (Cpb) |
| SPs0101 | | LepA putative signal peptidase I |
| SPs0102 | QVXTG | conserved hypothetical protein |
| | | (fimbrial) |
| SPs0103 | SrtC2 | sortase |
| SPs0104 | LPXAG | hypothetical protein |
| SPs0105 | | putative multiple sugar metabolism |
| | | regulator |
| SPs0106 | LPXTG | protein F2 like fibronectin-binding |
| | | protein |

TABLE 10
GAS AI-3 sequences from M5 isolate (Manfredo)

| | Sortase | |
|---|---|---|
| AI-3 | substrate | |
| sequence | sequence or | |
| identifier | sortase type | Functional description |
| orf77 | | Negative transcriptional regulator (Nra) |
| orf78 | VPXTG | putative collagen binding protein (Cpb) |
| orf79 | | LepA putative signal peptidase I |
| orf80 | QVXTG | conserved hypothetical protein |
| | | (fimbrial) |
| orf81 | SrtC2 | sortase |
| orf82 | LPXAG | hypothetical protein |
| orf83 | | putative multiple sugar metabolism |
| | | regulator |
| orf84 | LPXTG | protein F2 like fibronectin-binding |
| | | protein |

TABLE 8
GAS AI-3 sequences from M18 isolate (MGAS8232)

| | Sortase | |
|---|---|---|
| AI-3 | substrate | |
| sequence | sequence or | |
| identifier | sortase type | Functional description |
| spyM18_0125 | | Negative transcriptional regulator (Nra) |
| | | (N-terminal fragment) |
| spyM18_0126 | VPXTG | putative collagen binding protein (Cpb) |
| spyM18_0127 | | LepA putative signal peptidase I |
| spyM18_0128 | QVXTG | conserved hypothetical protein |
| | | (fimbrial) |
| SpyM18_0129 | SrtC2 | sortase |
| spyM18_0130 | LPXAG | hypothetical protein |
| spyM18_0131 | | putative multiple sugar metabolism |
| | | regulator |
| spyM18_0132 | LPXTG | protein F2 like fibronectin-binding |
| | | protein |

TABLE 44
GAS AI-3 sequences from M49 isolate (591)

| | Sortase | |
|---|---|---|
| AI-3 | substrate | |
| sequence | sequence or | |
| identifier | sortase type | Functional description |
| SpyoM01000156 | | Negative transcriptional regulator (Nra) |
| SpyoM01000155 | VPXTG | collagen binding protein (Cpa) |
| SpyoM01000154 | | LepA putative signal peptidase I |
| SpyoM01000153 | QVXTG | conserved hypothetical protein |
| | | (fimbrial) |
| SpyoM01000152 | SrtC2 | sortase |
| SpyoM01000151 | LPXAG | hypothetical protein |
| SpyoM01000150 | | MsmRL |
| SpyoM01000149 | LPXTG | protein F2 like fibronectin-binding |
| | | protein |

A schematic of AI-3 serotypes M3, M5, M18, and M49 is shown in FIG. 51A. Each contains an open reading frame encoding a SrtC2-type sortase of nearly identical amino acid sequence. See FIG. 52B for an amino acid sequence alignment for each of the SrtC2 amino acid sequences.

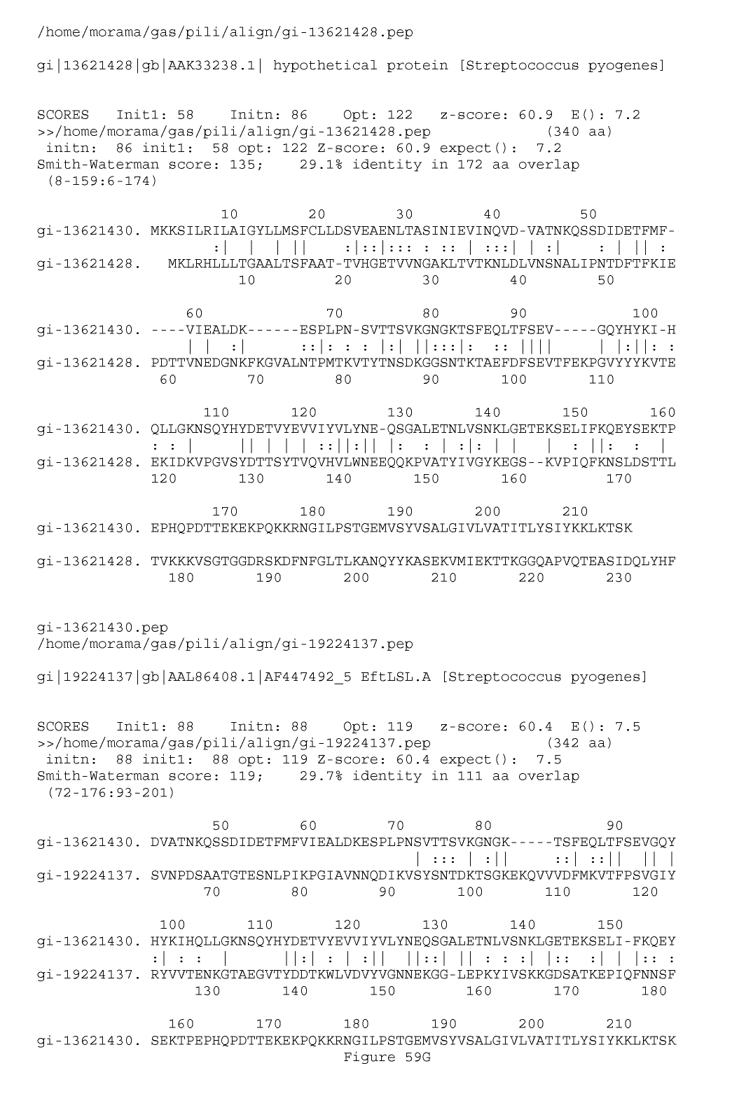

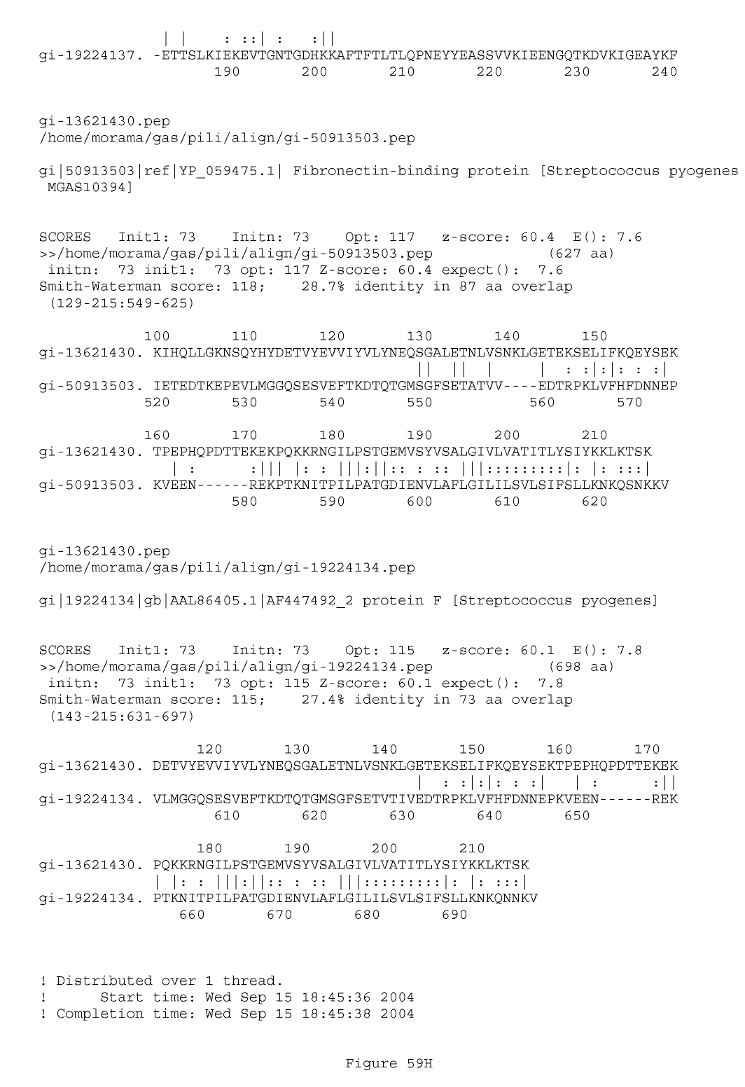

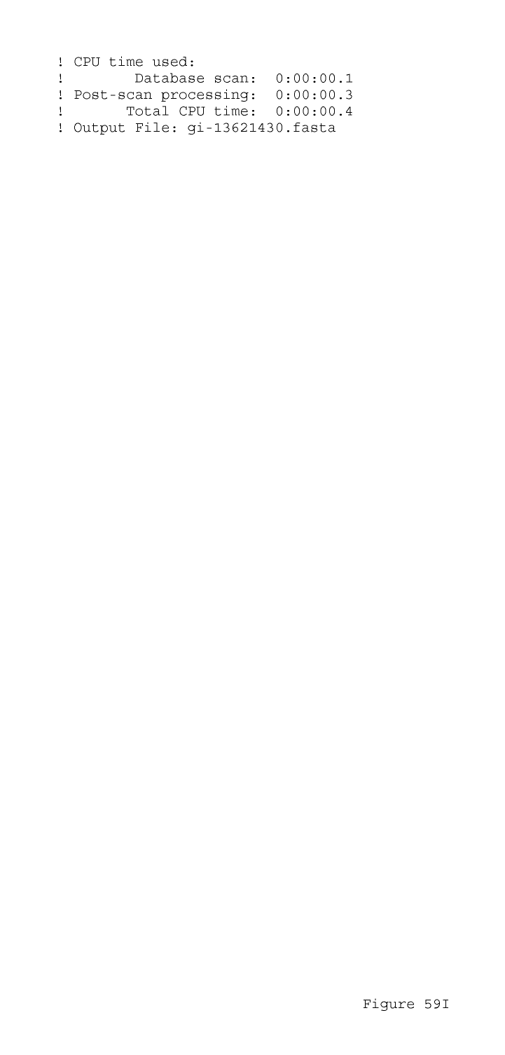

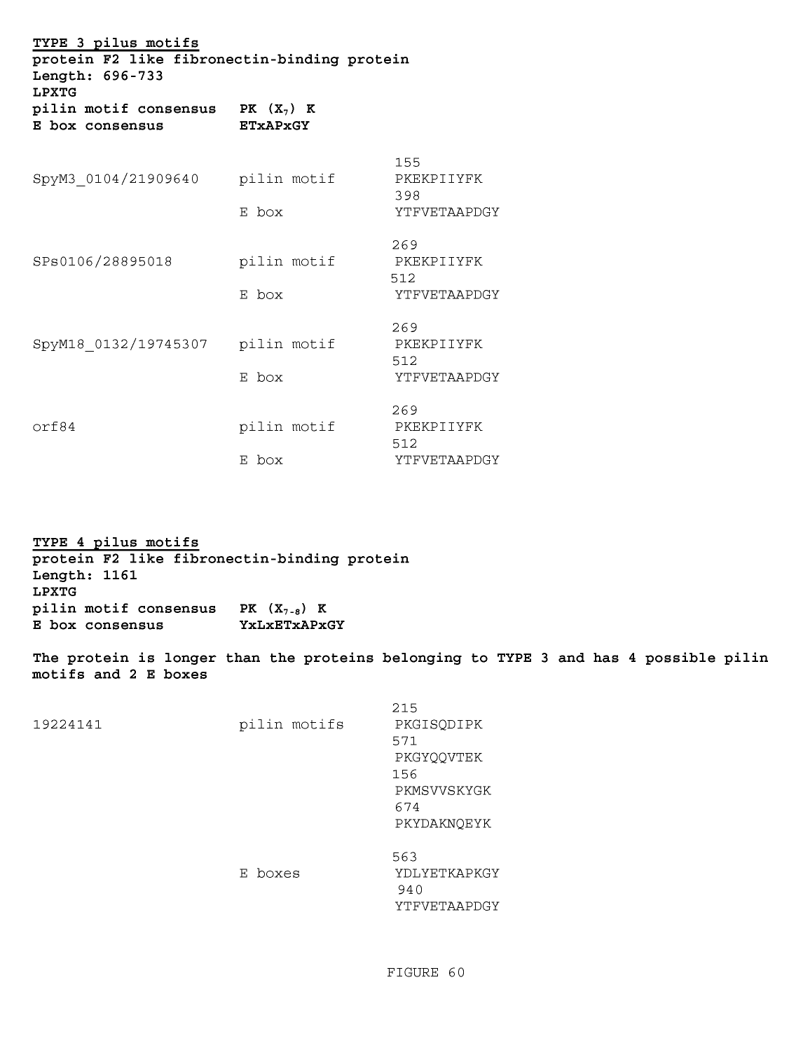

The protein F2-like fibronectin-binding protein of each these type 3 adhesin islands contains a pilin motif and an E-box. FIG. 60 indicates the amino acid sequence of the pilin motif and E-box of each of GAS AI-3 serotype M3 MGAS315 (SpyM3_{—}0104/21909640), GAS AI-3 serotype M3 SSI (Sps0106/28895018), GAS AI-3 serotype M18 (SpyM18_{—}0132/19745307), and GASAI-3 serotype M5 (orf84).

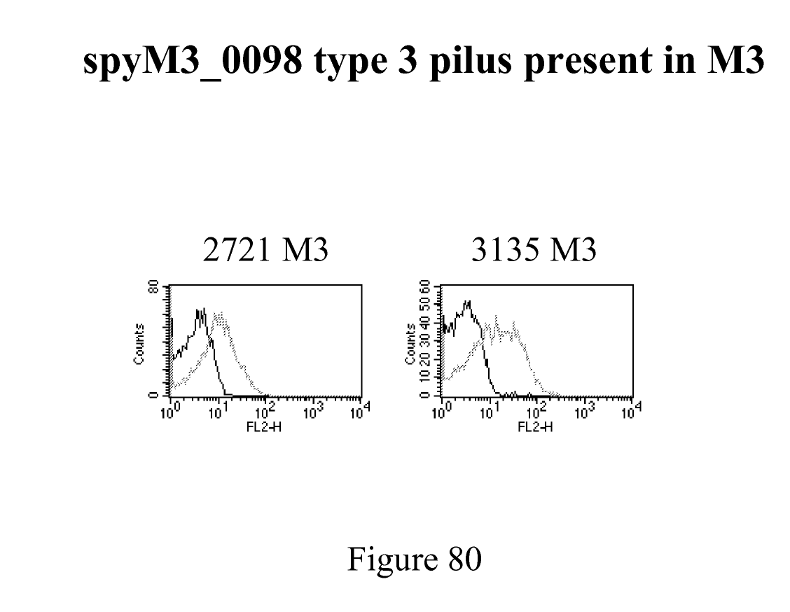

FACS analysis has confirmed that the GAS AI-3 surface proteins SpyM3_{—}0098, SpyM3_{—}0100, SpyM3_{—}0102, and SpyM3_{—}0104 are expressed on the surface of GAS. FIG. 80 provides the results of FACS analysis for surface expression of SpyM3_{—}0098 on each of GAS serotypes M3 2721 and M3 3135. A shift in fluorescence is observed for each GAS serotype when anti-SpyM3_{—}0098 antiserum is present, demonstrating cell surface expression. Table 25, below, quantitatively summarizes the FACS fluorescence values obtained for each GAS serotype in the presence of pre-immune antiserum, anti-SpyM3_{—}0098 antiserum, and the difference in fluorescence value between the pre-immune and anti-SpyM3_{—}0098 antiserum.

TABLE 25
Summary of FACS values for surface expression of SpyM3_0098

| 2721 | | | 3135 | | |
|---|---|---|---|---|---|
| Pre- | Anti- | | Pre- | Anti- | |
| immune | spyM3_0098 | Change | immune | spyM3_0098 | Change |
| 117.85 | 249.51 | 132 | 99.17 | 277.21 | 178 |

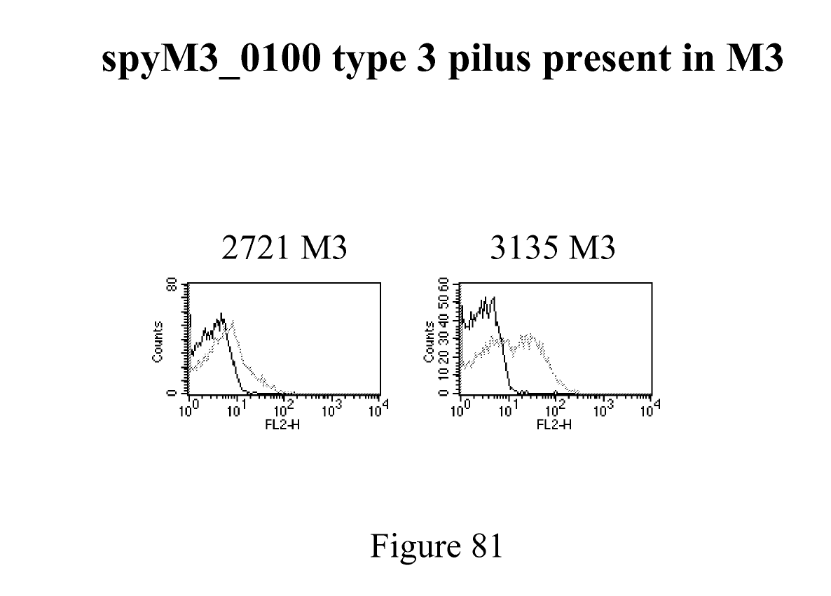

FIG. 81 provides the results of FACS analysis for surface expression of SpyM3_{—}0100 on each of GAS serotypes M3 2721 and M3 3135. A shift in fluorescence is observed for each GAS serotype when anti-SpyM3_{—}0100 antiserum is present, demonstrating cell surface expression. Table 26, below, quantitatively summarizes the FACS fluorescence values obtained for each GAS serotype in the presence of pre-immune antiserum, anti-SpyM3_{—}0100 antiserum, and the difference in fluorescence value between the pre-immune and anti-SpyM3_{—}0100 antiserum.

TABLE 26
Summary of FACS values for surface expression of SpyM3_0100

| 2721 | | | 3135 | | |
|---|---|---|---|---|---|
| Pre- | Anti- | | Pre- | Anti- | |
| immune | spyM3_0100 | Change | immune | spyM3_0100 | Change |
| 110.31 | 181.91 | 72 | 97.87 | 250.01 | 152 |

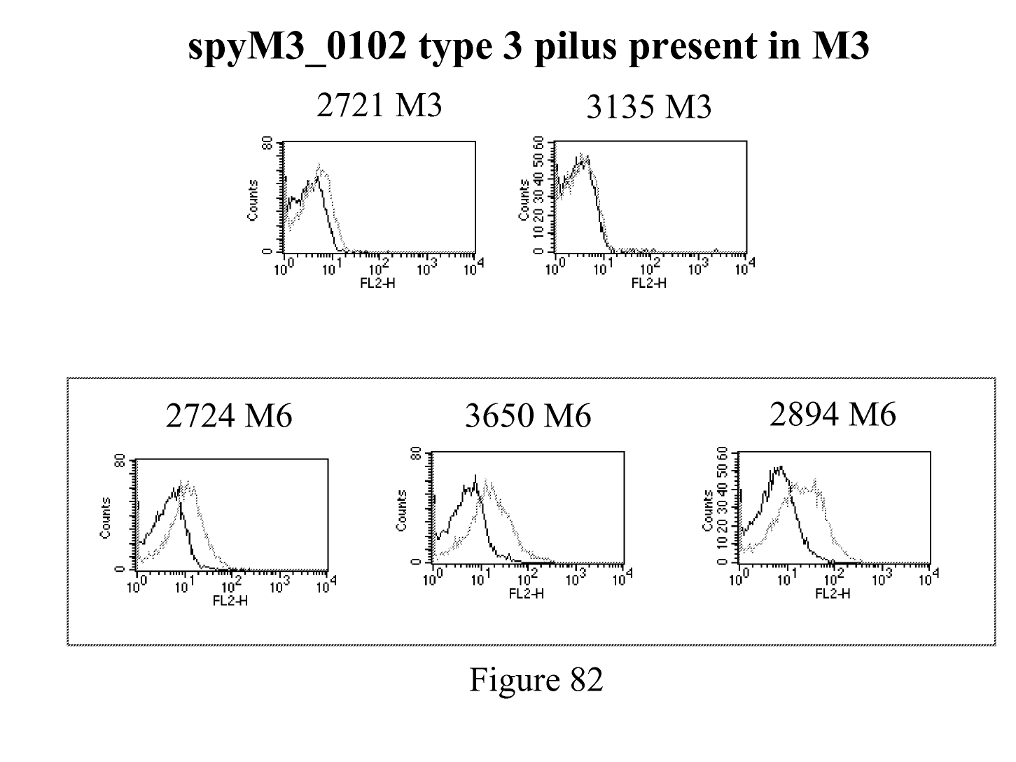

FIG. 82 provides the results of FACS analysis for surface expression of SpyM3_{—}0102 on each of GAS serotypes M3 2721 and M3 3135. A shift in fluorescence is observed for each GAS serotype when anti-SpyM3_{—}0102 antiserum is present, demonstrating cell surface expression. Table 27, below, quantitatively summarizes the FACS fluorescence values obtained for each GAS serotype in the presence of pre-immune antiserum, anti-SpyM3_{—}0102 antiserum, and the difference in fluorescence value between the pre-immune and anti-SpyM3_{—}0102 antiserum.

TABLE 27
Summary of FACS values for surface expression of SpyM3_0102
in M3 serotypes

| 2721 | | | 3135 | | |
|---|---|---|---|---|---|
| Pre- | Anti- | | Pre- | Anti- | |
| immune | spyM3_0102 | Change | immune | spyM3_0102 | Change |
| 109.86 | 155.26 | 45 | 100.02 | 112.58 | 13 |

FIG. 82 also provides the results of FACS analysis for surface expression of a pilin antigen that has homology to SpyM3_{—}0102 identified in a different GAS serotype, M6. FACS analysis conducted with the SpyM3_{—}0102 antisera was able to detect surface expression of the homologous SpyM3_{—}0102 antigen on each of GAS serotypes M6 2724, M6 3650, and M6 2894. Table 28, below, quantitatively summarizes the FACS fluorescence values obtained for each GAS serotype in the presence of pre-immune antiserum, anti-SpyM3_{—}0102 antiserum, and the difference in fluorescence value between the pre-immune and anti-SpyM3_{—}0102 antiserum.

TABLE 28
Summary of FACS values for surface expression of SpyM3_0102 in M6 serotypes

| 2724 | | | 3650 | | | 2894 | | |
|---|---|---|---|---|---|---|---|---|
| Pre- | Anti- | | Pre- | Anti- | | Pre- | Anti- | |
| immune | spyM3_0102 | Change | immune | spyM3_0102 | Change | immune | spyM3_0102 | Change |
| 146.59 | 254.03 | 107 | 162.56 | 294.03 | 131 | 175.49 | 313.69 | 138 |

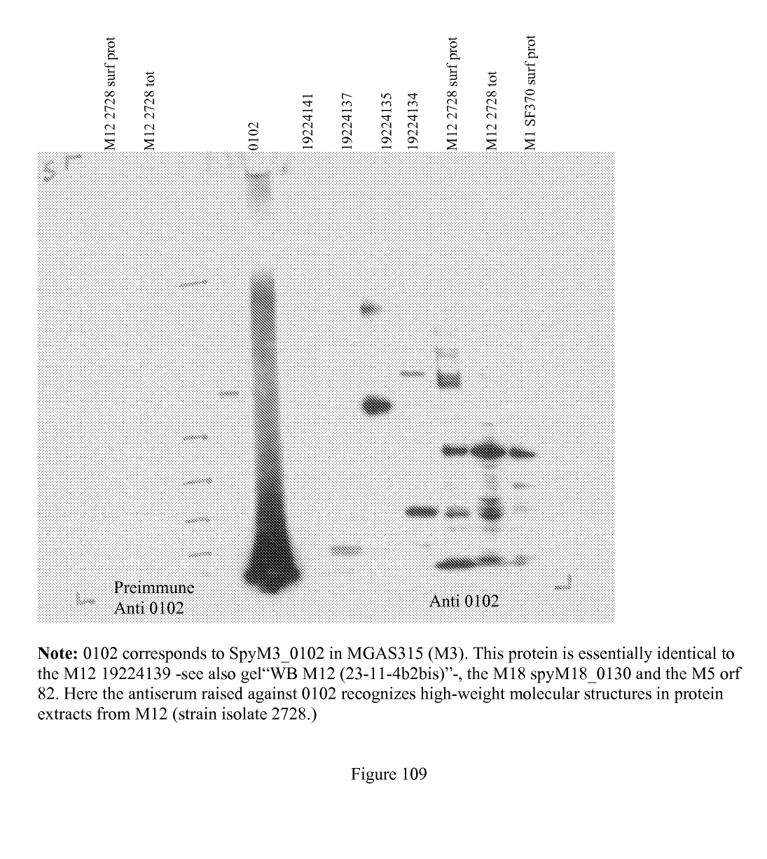

SpyM3_{—}0102 is also homologous to pilin antigen 19224139 of GAS serotype M12. Antisera raised against SpyM3_{—}0102 is able to detect high molecular weight structures in GAS serotype M12 strain 2728 protein fractions enriched for surface proteins, which would contain the 19224139 antigen. See FIG. 109 at the lane labelled M12 2728 surf prot.

FIG. 83 provides the results of FACS analysis for surface expression of SpyM3_{—}0104 on each of GAS serotypes M3 2721 and M3 3135. A shift in fluorescence is observed for each GAS serotype when anti-SpyM3_{—}0104 antiserum is present, demonstrating cell surface expression. Table 29, below, quantitatively summarizes the FACS fluorescence values obtained for each GAS serotype in the presence of pre-immune antiserum, anti-SpyM3_{—}0104 antiserum, and the difference in fluorescence value between the pre-immune and anti-SpyM3_{—}0104 antiserum.

TABLE 29
Summary of FACS values for surface expression of SpyM3_0104
in M3 serotypes

| 2721 | | | 3135 | | |
|---|---|---|---|---|---|
| Pre- | Anti- | | Pre- | Anti- | |
| immune | spyM3_0104 | Change | immune | spyM3_0104 | Change |
| 128.45 | 351.65 | 223 | 105.1 | 339.88 | 235 |

FIG. 83 also provides the results of FACS analysis for surface expression of a pilin antigen that has homology to SpyM3_{—}0104 identified in a different GAS serotype, M12. FACS analysis conducted with the SpyM3_{—}0104 antisera was able to detect surface expression of the homologous SpyM3_{—}0104 antigen on GAS serotype M12 2728. Table 30, below, quantitatively summarizes the FACS fluorescence values obtained for this GAS serotype in the presence of pre-immune antiserum, anti-SpyM3_{—}0104 antiserum, and the difference in fluorescence value between the pre-immune and anti-SpyM3_{—}0104 antiserum.

TABLE 30
Summary of FACS values for surface expression
of SpyM3_0104 in an M12 serotype
2728

| Pre-immune | Anti-spyM3_0104 | Change |
|---|---|---|
| 198.57 | 288.75 | 90 |

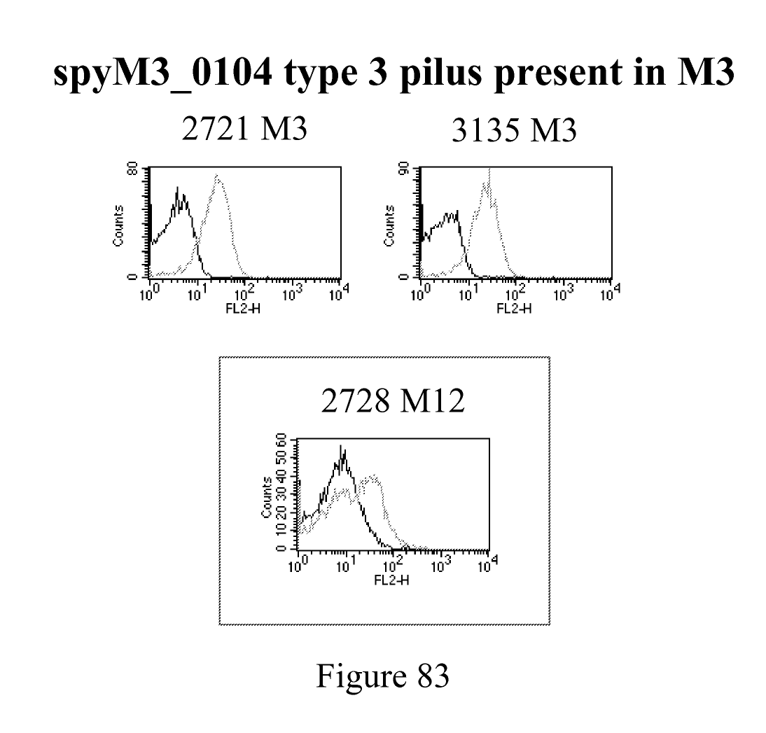

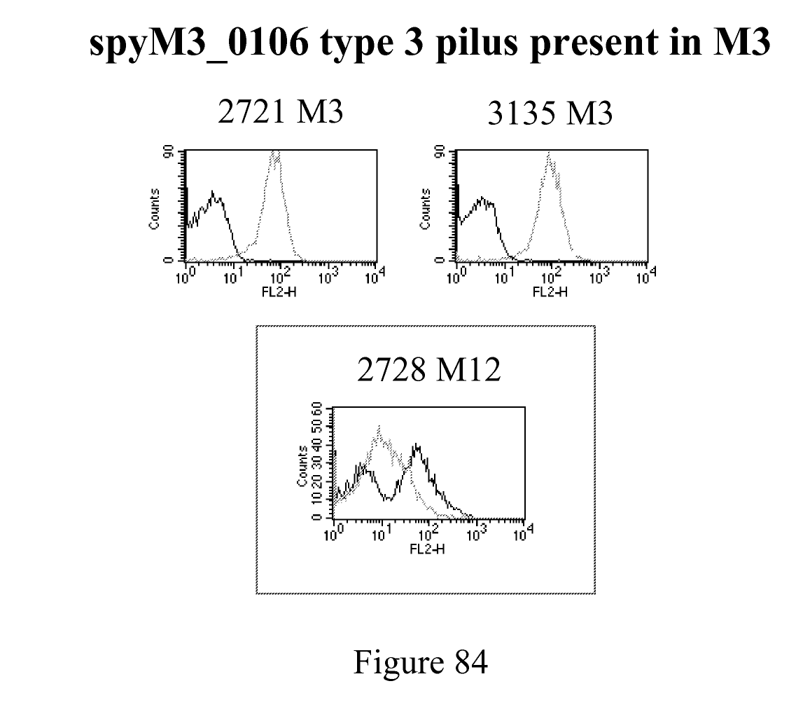

FIG. 84 provides the results of FACS analysis for surface expression of SPs_{—}0106 on each of GAS serotypes M3 2721 and M3 3135. A shift in fluorescence is observed for each GAS serotype when anti-SPs_{—}0106 antiserum is present, demonstrating cell surface expression. Table 31, below, quantitatively summarizes the FACS fluorescence values obtained for each GAS serotype in the presence of pre-immune antiserum, anti-SPs_{—}0106 antiserum, and the difference in fluorescence value between the pre-immune and anti-SPs_{—}0106 antiserum.

TABLE 31
Summary of FACS values for surface expression of
SPs_0106 in M3 serotypes

| 2721 | | | 3135 | | |
|---|---|---|---|---|---|
| | Anti- | | | Anti- | |
| Pre-immune | SPs_0106 | Change | Pre-immune | SPs_0106 | Change |
| 116 | 463.28 | 347 | 103.02 | 494.27 | 391 |

FIG. 84 also provides the results of FACS analysis for surface expression of a pilin antigen that has homology to SPs_{—}0106 identified in a different GAS serotype, M12. FACS analysis conducted with the SPs_{—}0106 antisera was able to detect surface expression of the homologous SPs_{—}0106 antigen on GAS serotype M12 2728. Table 32, below, quantitatively summarizes the FACS fluorescence values obtained for each GAS serotype in the presence of pre-immune antiserum, anti-SPs_{—}0106 antiserum, and the difference in fluorescence value between the pre-immune and anti-SPs_{—}0106 antiserum.

TABLE 32
Summary of FACS values for surface expression
of SPs_0106 in an M12 serotype
2728

| Pre-immune | Anti-SPs_0106 | Change |
|---|---|---|
| 304.01 | 254.64 | −49 |

(4) Adhesin Island Sequence Within M12: GAS Adhesin Island 4 (“GAS AI-4”)

GAS Adhesin Island sequences within M12 serotype are outlined in Table 11 below. This GAS adhesin island 4 (“GAS AI-4”) comprises surface proteins, a SrtC2 sortase, and a RofA regulatory protein.

GAS AI-4 surface proteins within may include a fimbrial protein, an F or F2 like fibronectin-binding protein, and a capsular polysaccharide adhesion protein (Cpa). GAS AI-4 surface proteins may also include a hypothetical surface protein in an open reading frame (orf). Preferably, each of these GAS AI-4 surface proteins include an LPXTG sortase substrate motif, such as LPXTG (SEQ ID NO:122), VPXTG (SEQ ID NO:137), QVXTG (SEQ ID NO:138) or LPXAG (SEQ ID NO:139). GAS AI-4 includes a SrtC2 type sortase. GAS SrtC2 type sortases may preferably anchor surface proteins with a QVPTG (SEQ ID NO:140) motif, particularly when the motif is followed by a hydrophobic region and a charged C terminus tail. GAS AI-4 may also include a LepA putative signal peptidase I protein and a MsmRL protein.

TABLE 11
GAS AI-4 sequences from M12 isolate (A735)

| | Sortase | |
|---|---|---|
| AI-4 | substrate | |
| sequence | sequence or | |
| identifier | sortase type | Functional description |
| 19224133 | | RofA regulatory protein |
| 19224134 | LPXTG | protein F |
| | SrtB | SrtB (stop codon*) |
| 19224135 | VPXTG | Cpa |
| 19224136 | | LepA |
| 19224137 | QVXTG | EftLSL.A (fimbrial) |
| 19224138 | SrtC2 | EftLSL.B |
| 19224139 | LPXAG | Orf2 |
| 19224140 | | MsmRL |
| 19224141 | LPXTG | protein F2 |

A schematic of AI-4 serotype M12 is shown in FIG. 51A.

One of the open reading frames encodes a SrtC2-type sortase having an amino acid sequence nearly identical to the amino acid sequence of the SrtC2-type sortase of the AI-3 serotypes described above. See FIG. 52B for an amino acid sequence alignment for each of the SrtC2 amino acid sequences.

Other proteins encoded by the open reading frames of the AI-4 serotype M12 are homologous to proteins encoded by other known adhesin islands in S. pyogenes, as well as the GAS AI-3 serotype M5 (Manfredo). FIG. 52 is an amino acid alignment of the capsular polysaccharide adhesion protein (cpa) of AI-4 serotype M12 (19224135), GAS AI-3 serotype M5 (ORF78), S. pyogenes strain MGAS315 serotype M3 (21909634), S. pyogenes SSI-1 serotype M3 (28810257), S. pyogenes MGAS8232 serotype M3 (19745301), and GAS AI-2 serotype M1 (GAS15). The amino acid sequence of the AI-4 serotype M12 cpa shares a high degree of homology with other cpa proteins.

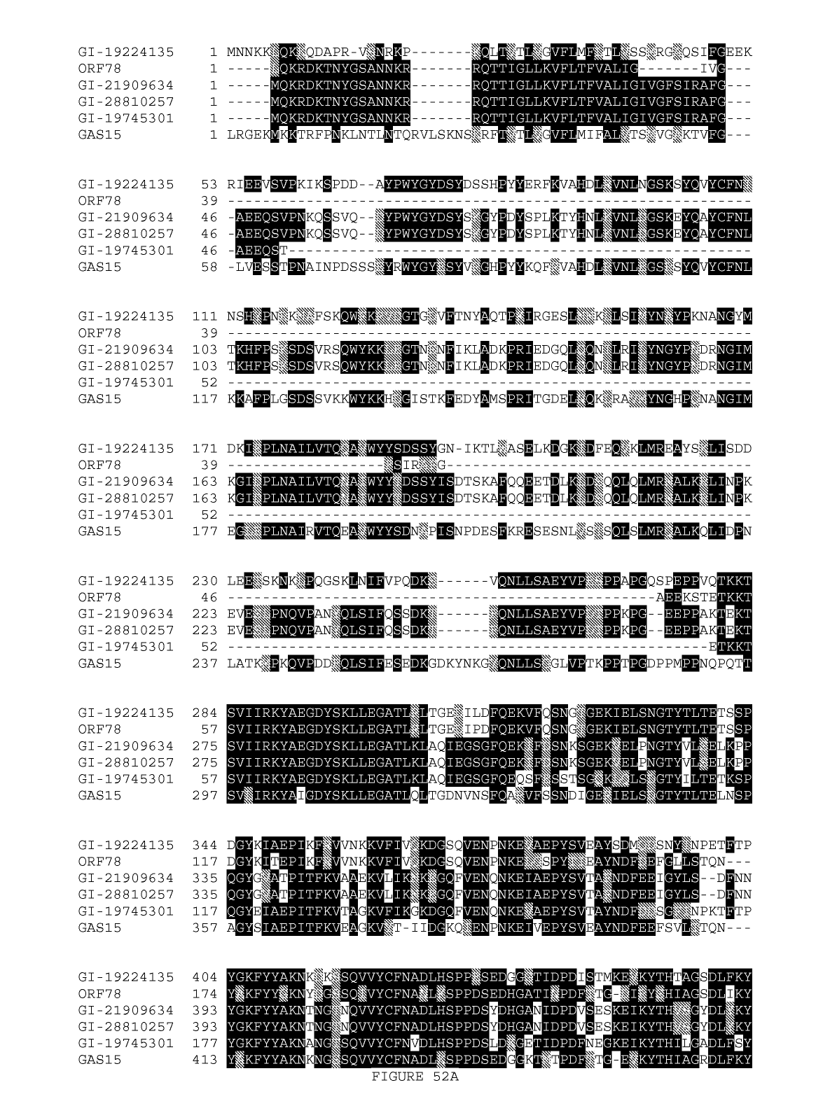

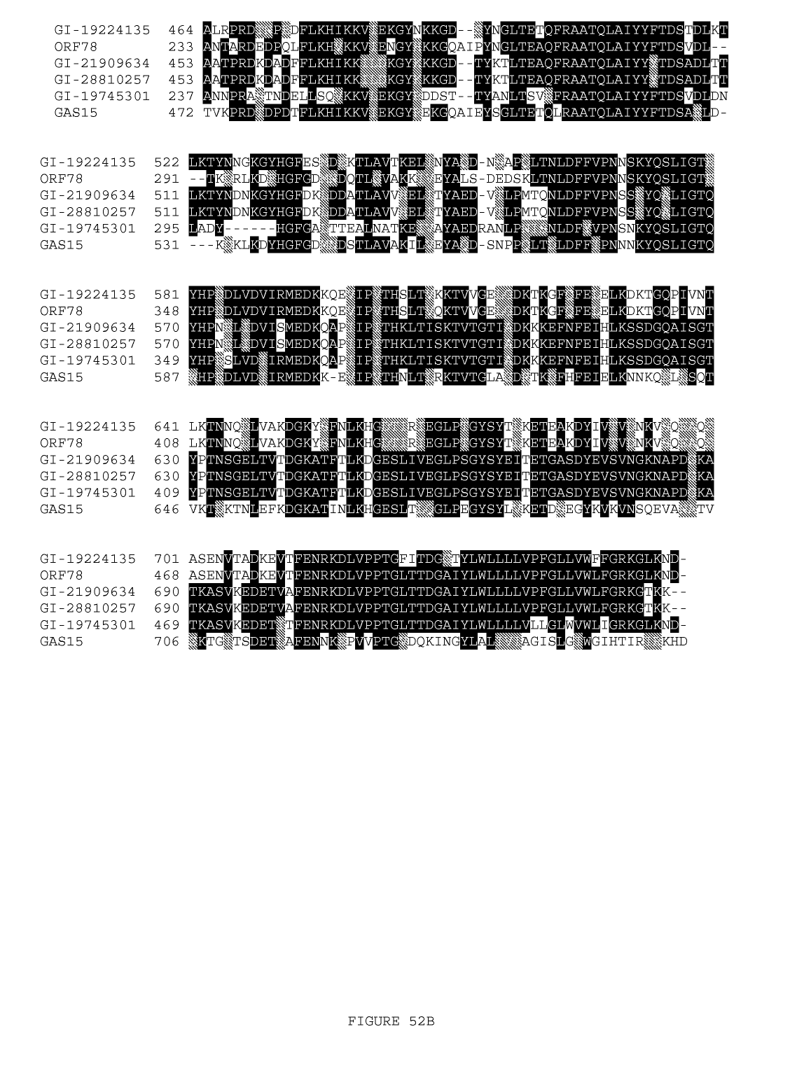

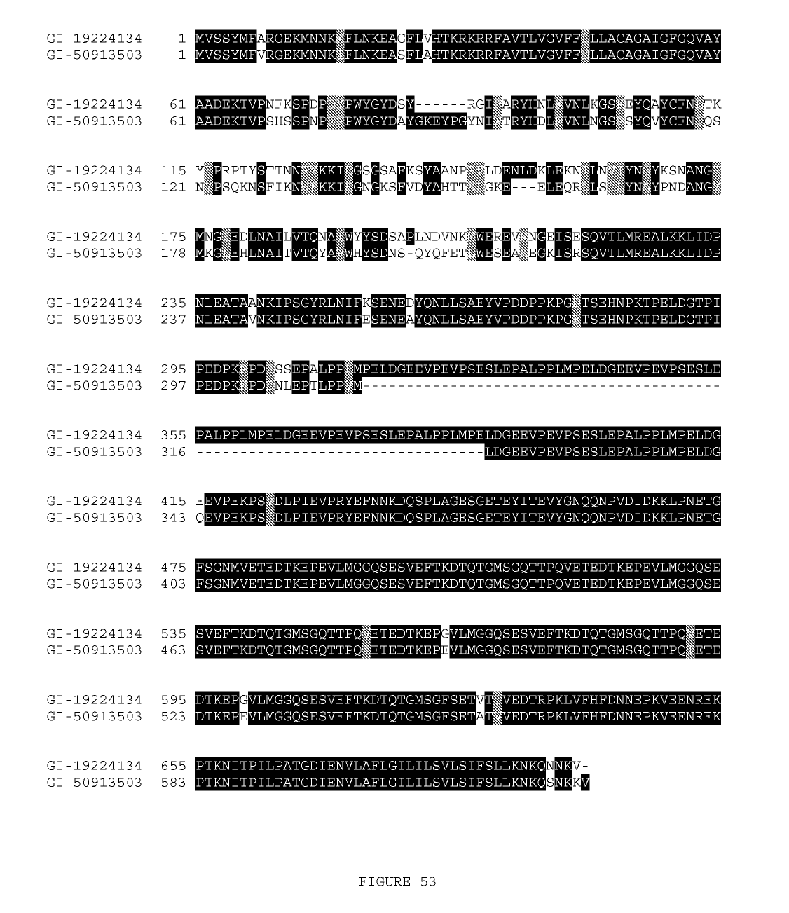

FIG. 53 shows that the F-like fibronectin-binding protein encoded by the AI-4 serotype M12 open reading frame (19224134) shares homology with a F-like fibronectin-binding protein found in S. pyogenes strain MGAS10394 serotype M6 (50913503).

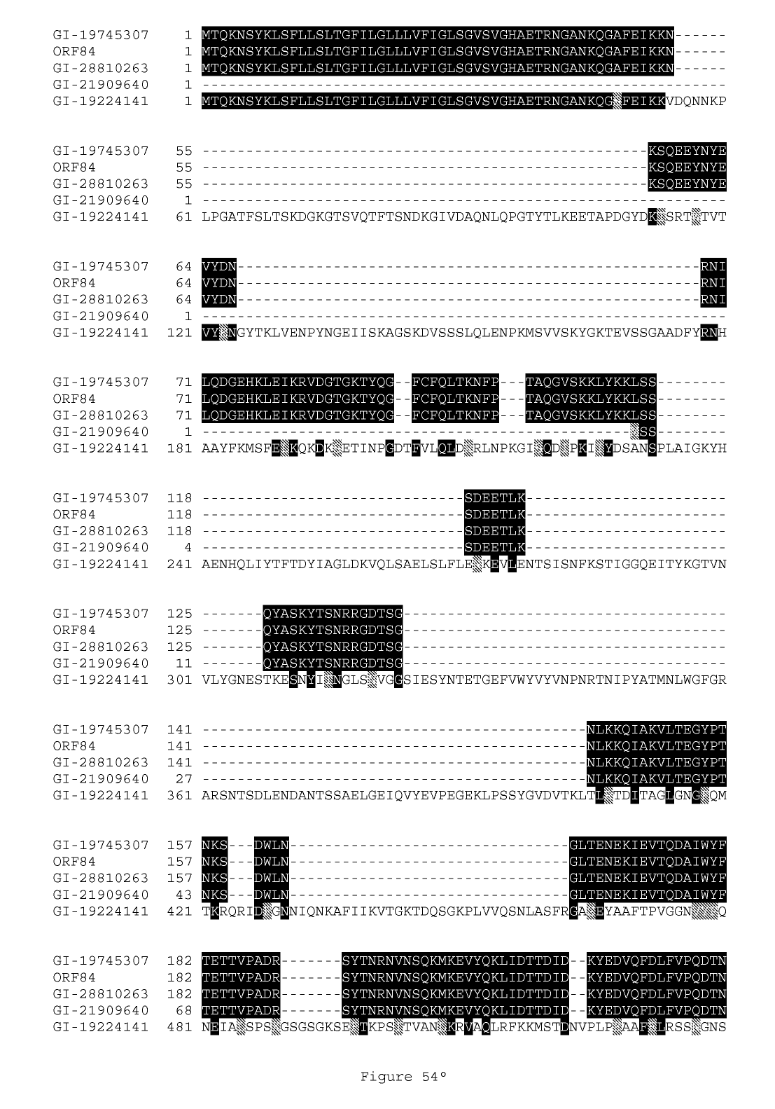

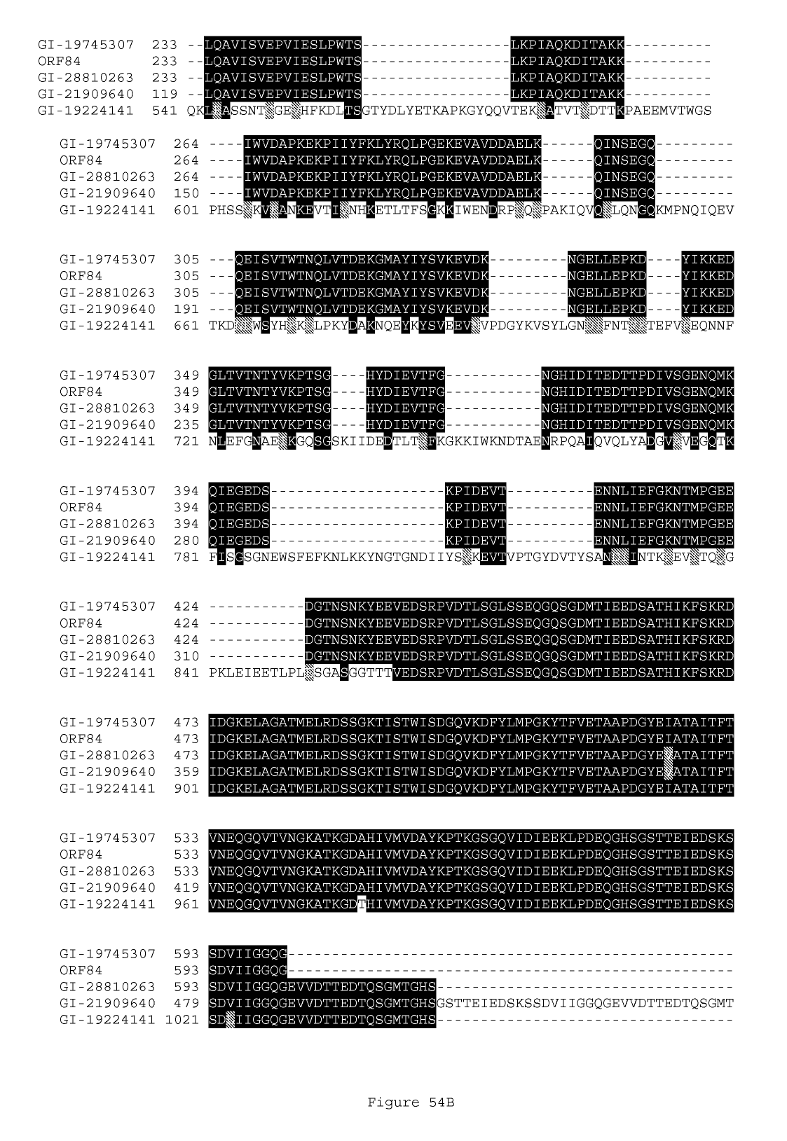

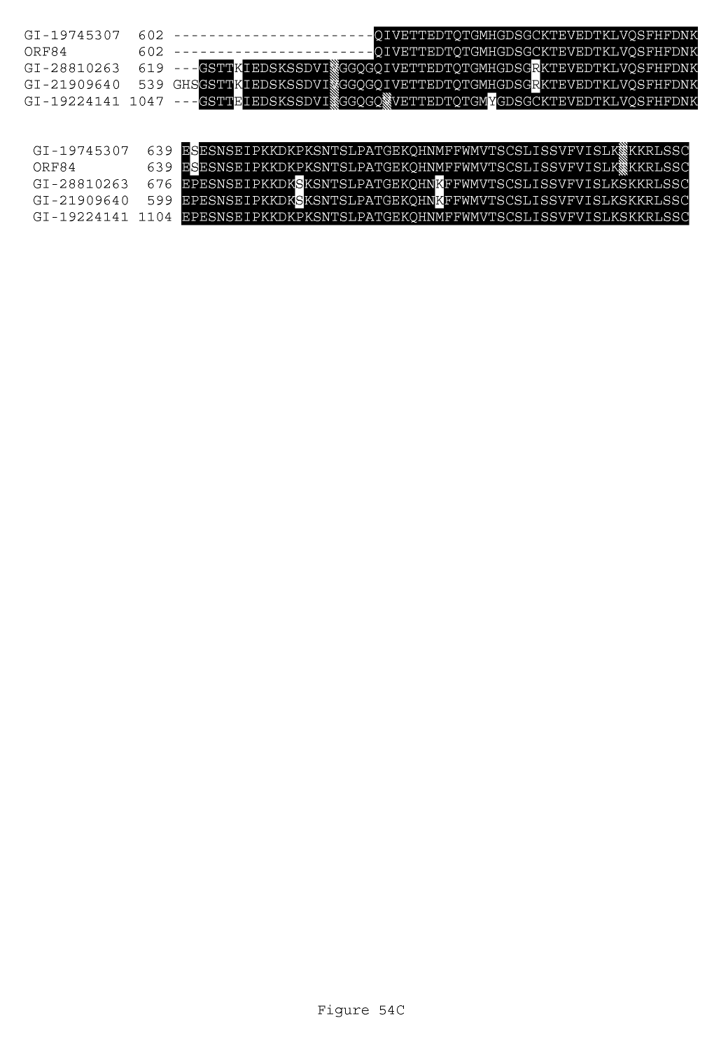

FIG. 54 is an amino acid sequence alignment that illustrates that the F2-like fibronectin-binding protein of AI-4 serotype M12 (19224141) shares homology with the F2-like fibronectin-binding protein of S. pyogenes strain MGAS8232 serotype M3 (19745307), GAS AI-3 serotype M5 (ORF84), S. pyogenes strain SSI serotype M3 (28810263), and S. pyogenes strain MGAS315 serotype M3 (21909640).

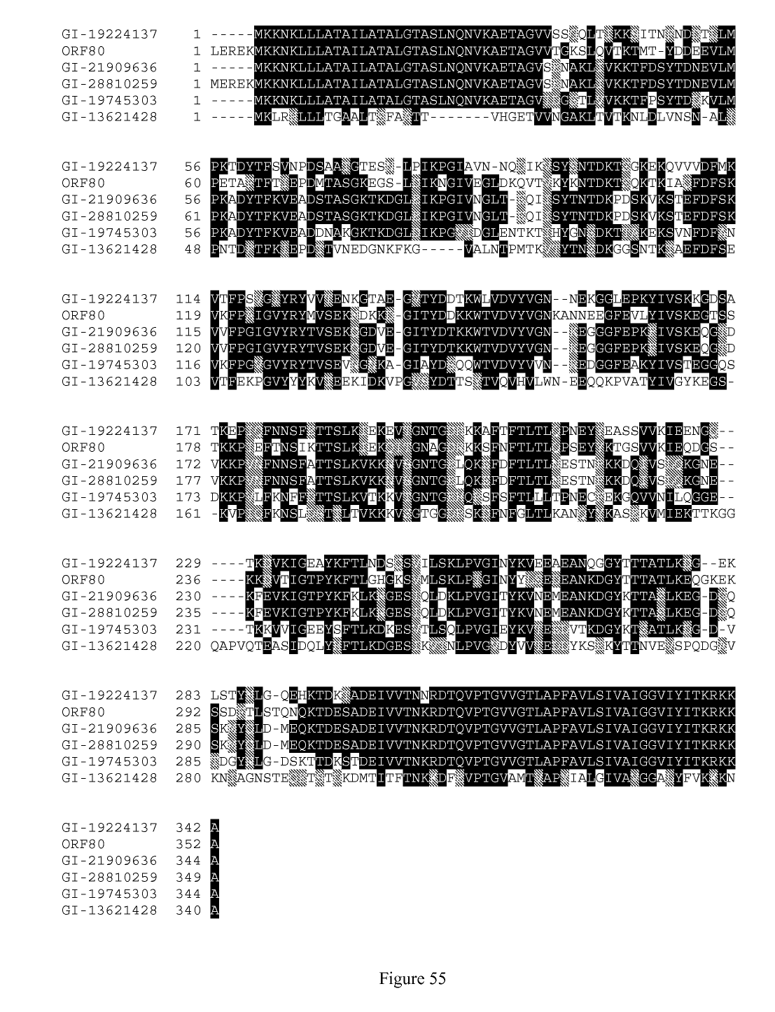

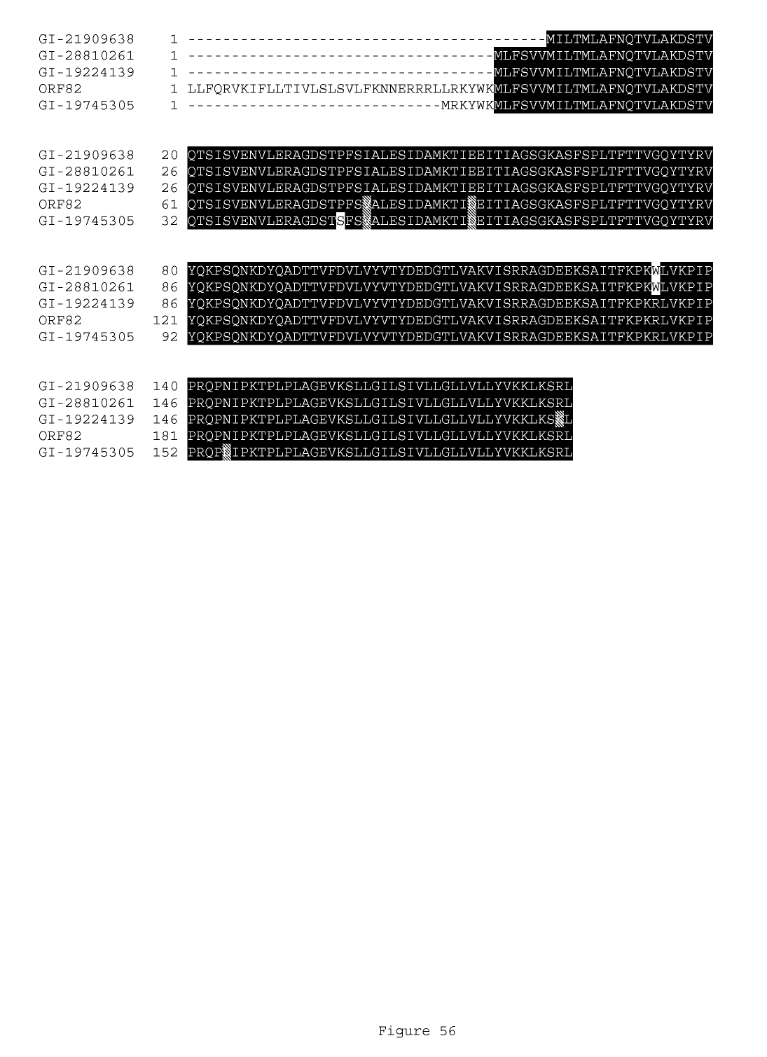

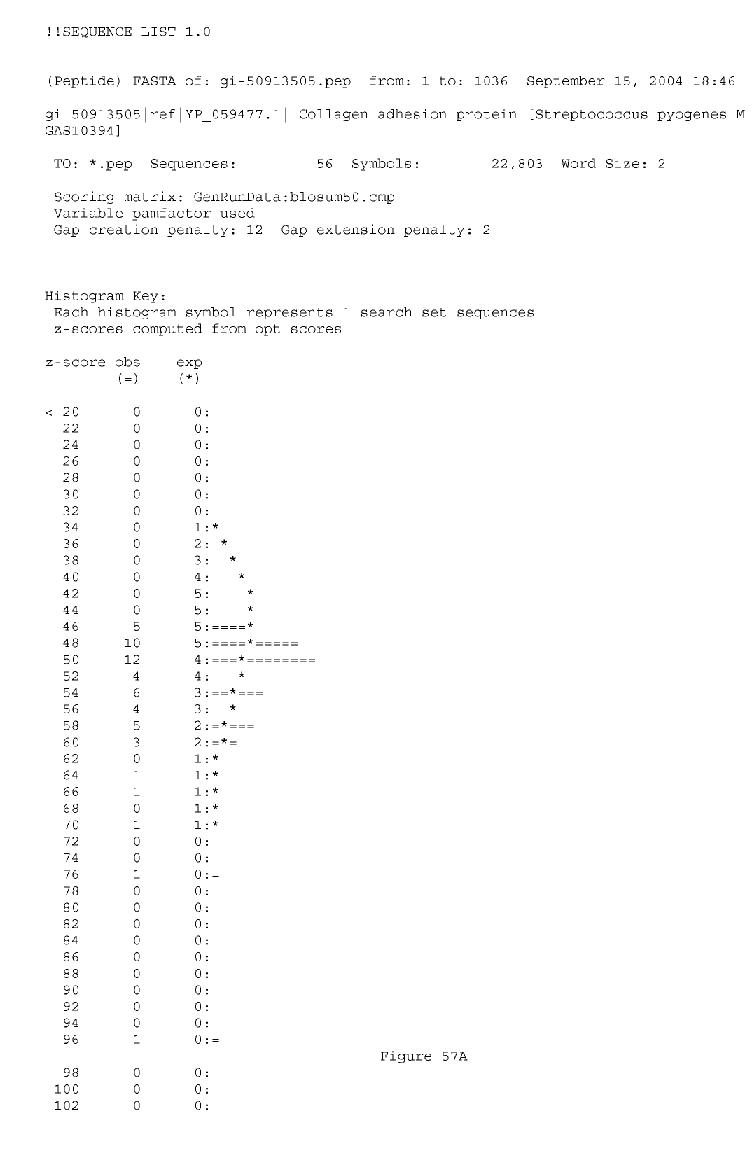

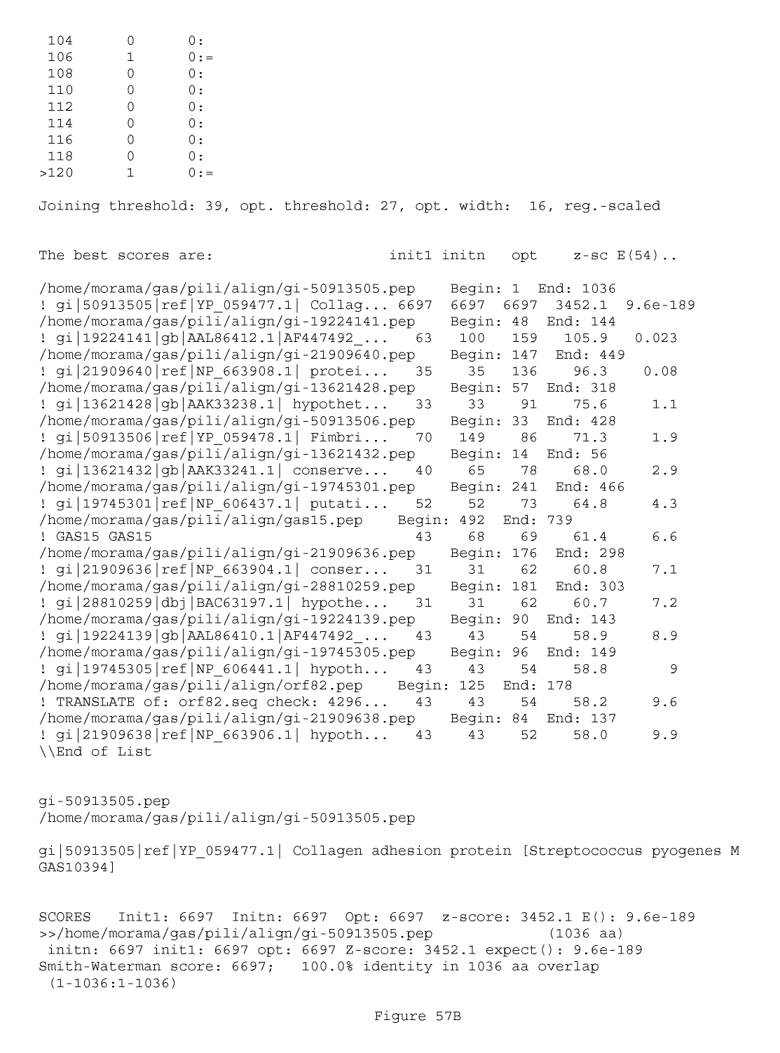

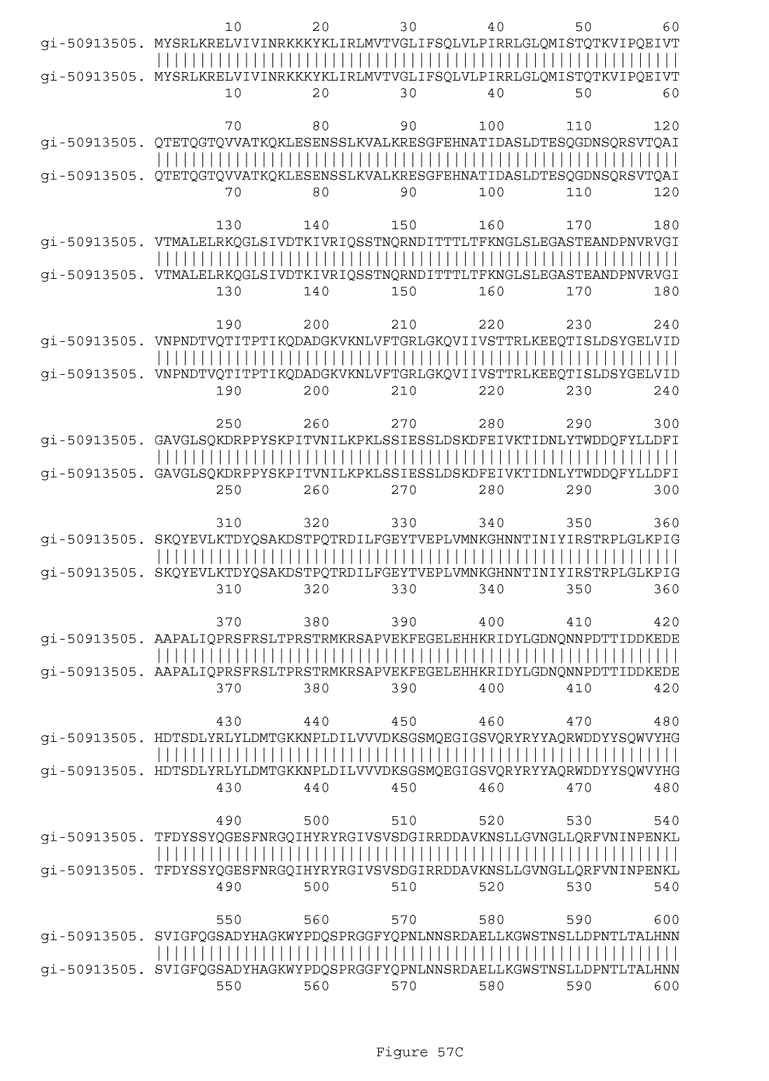

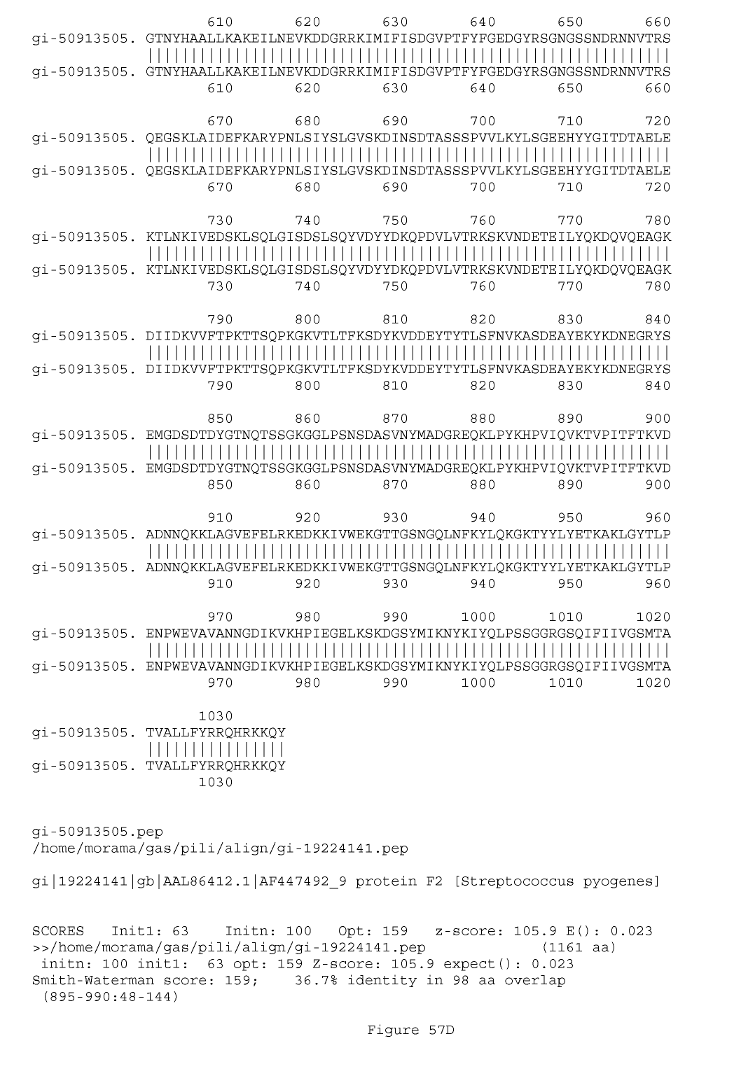

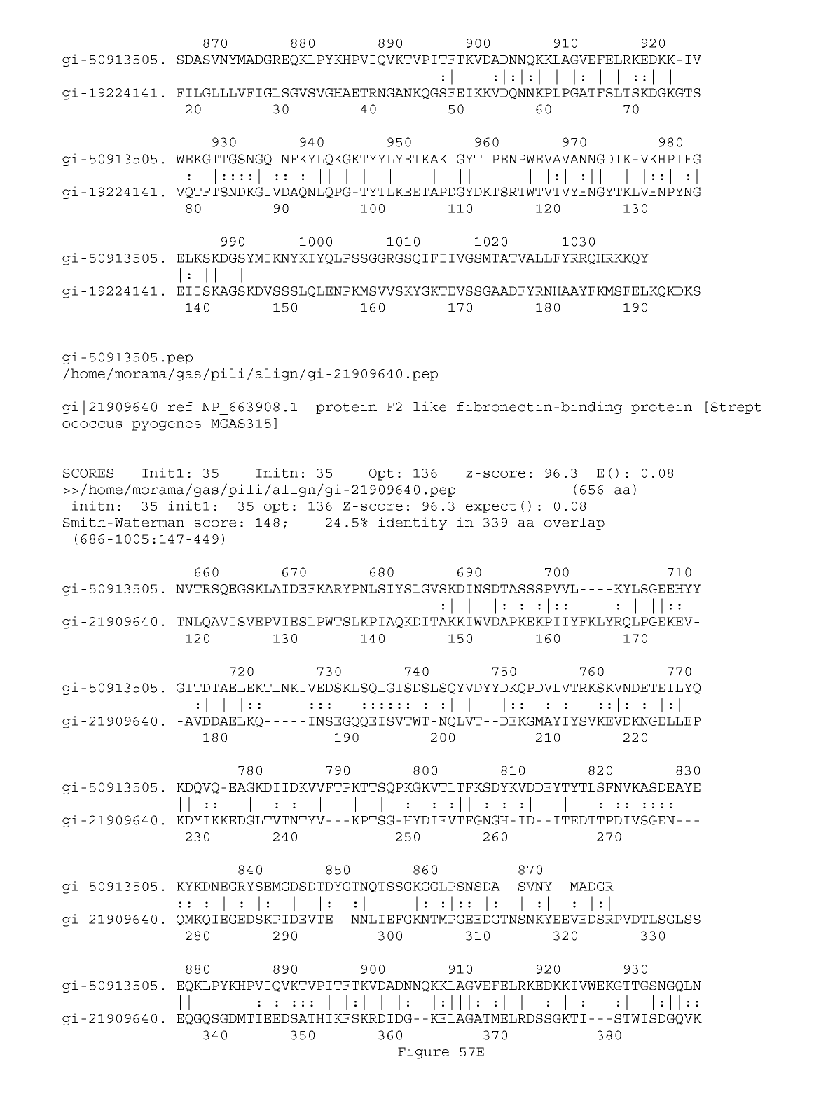

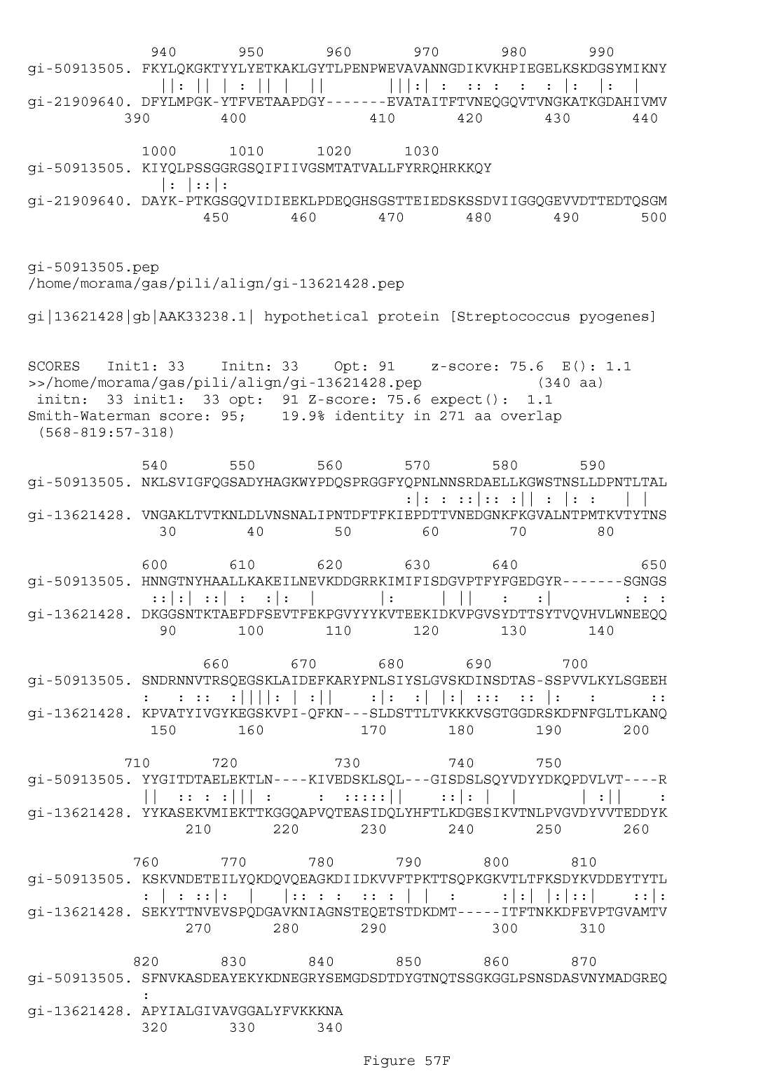

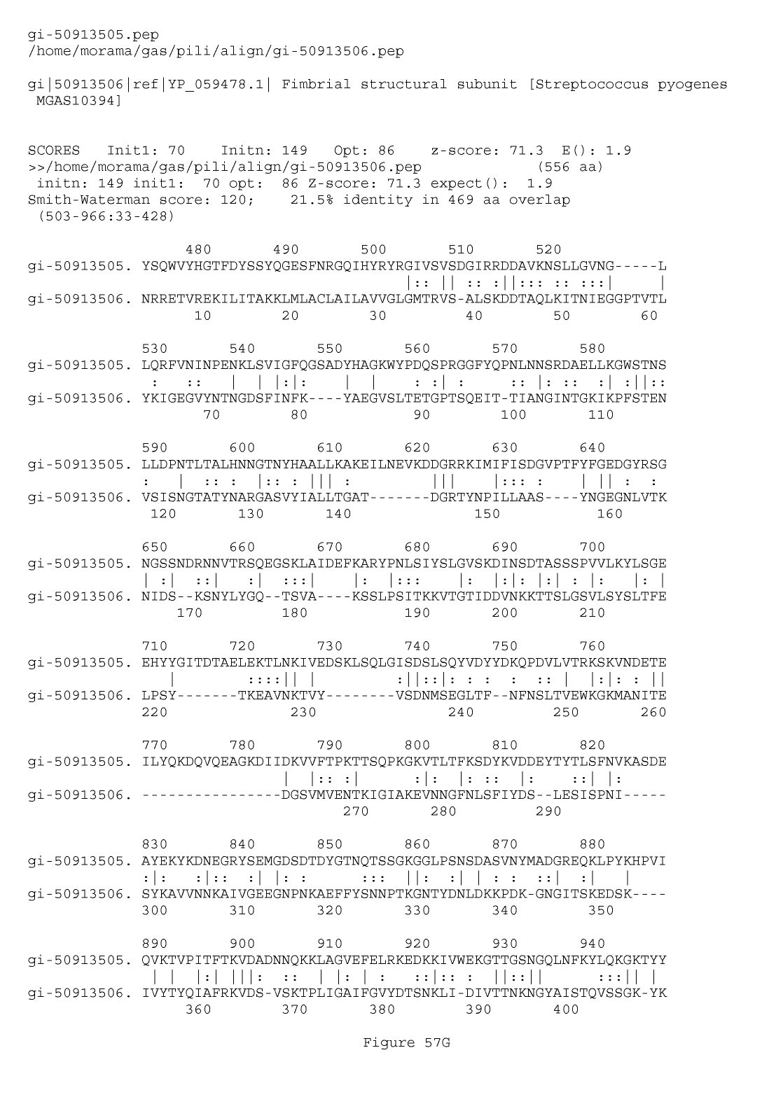

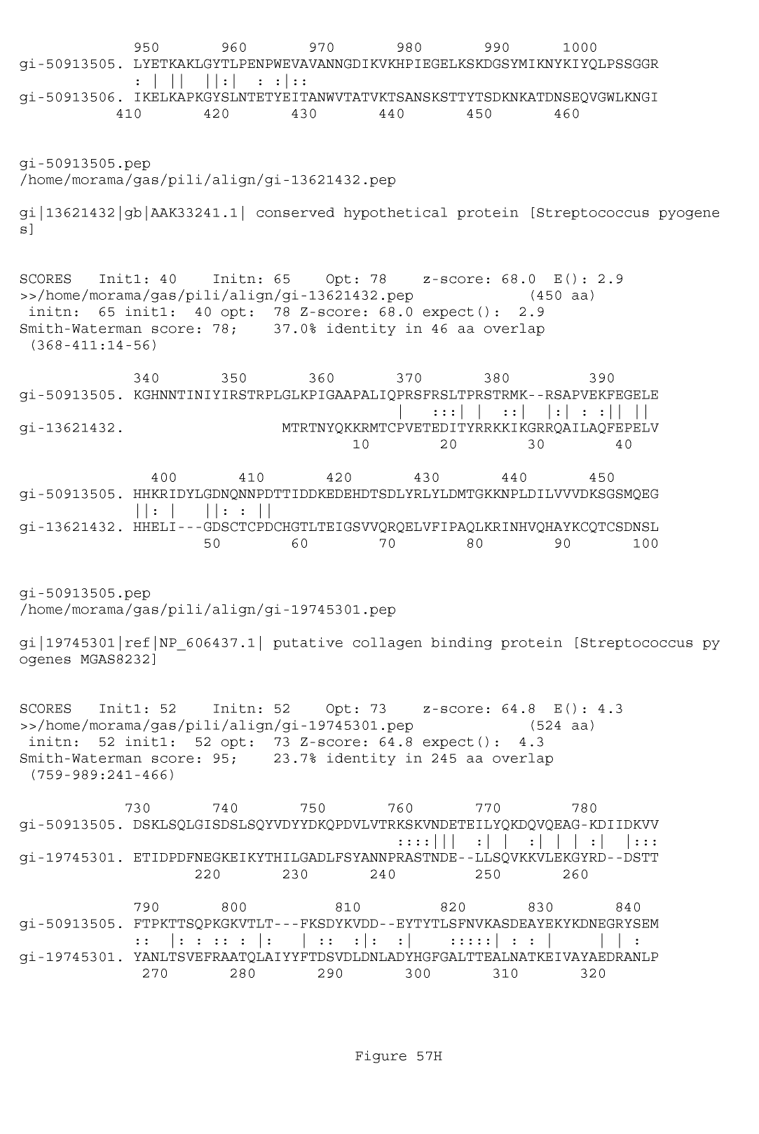

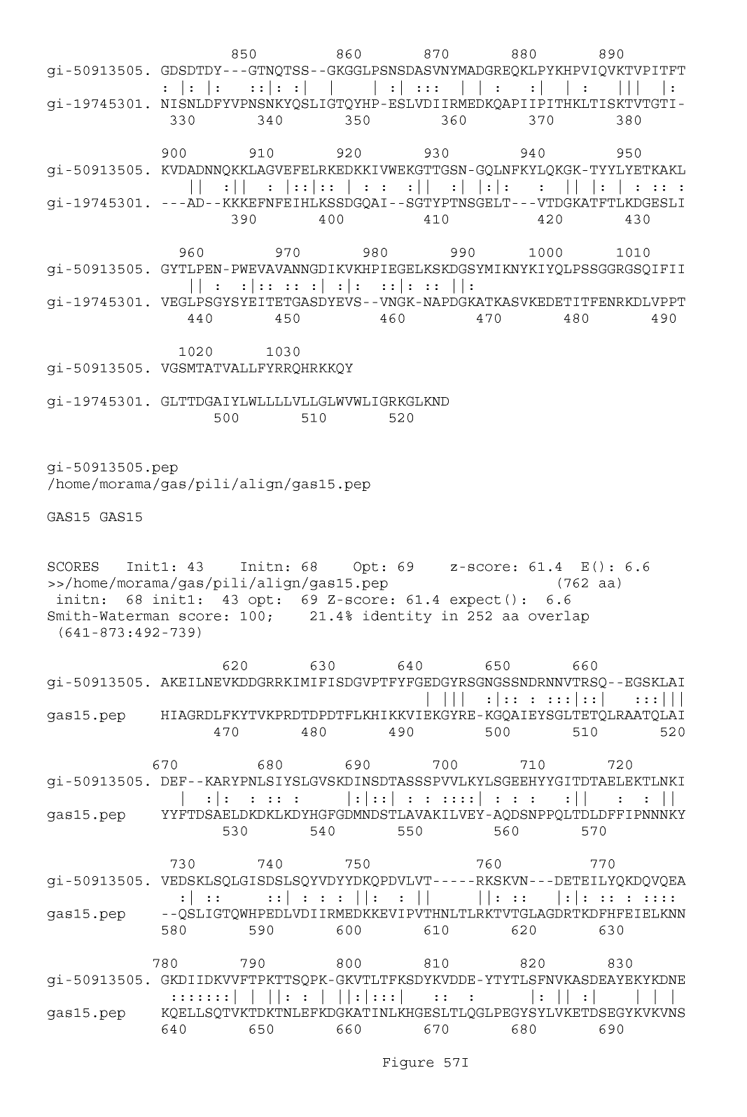

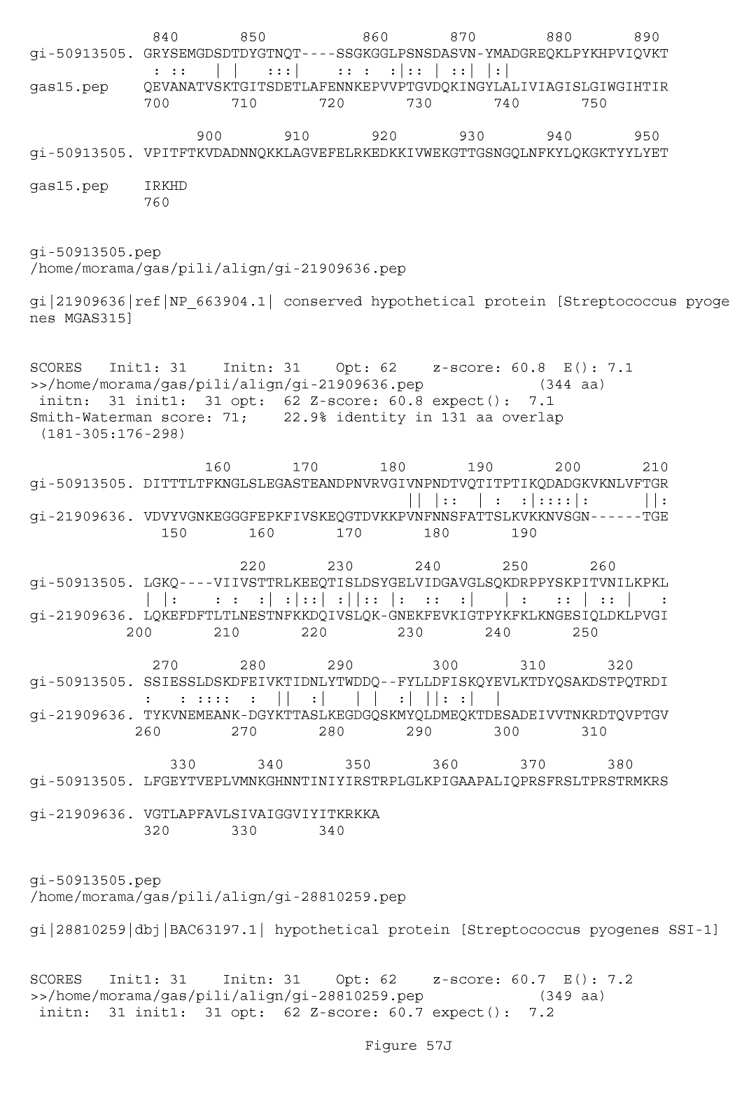

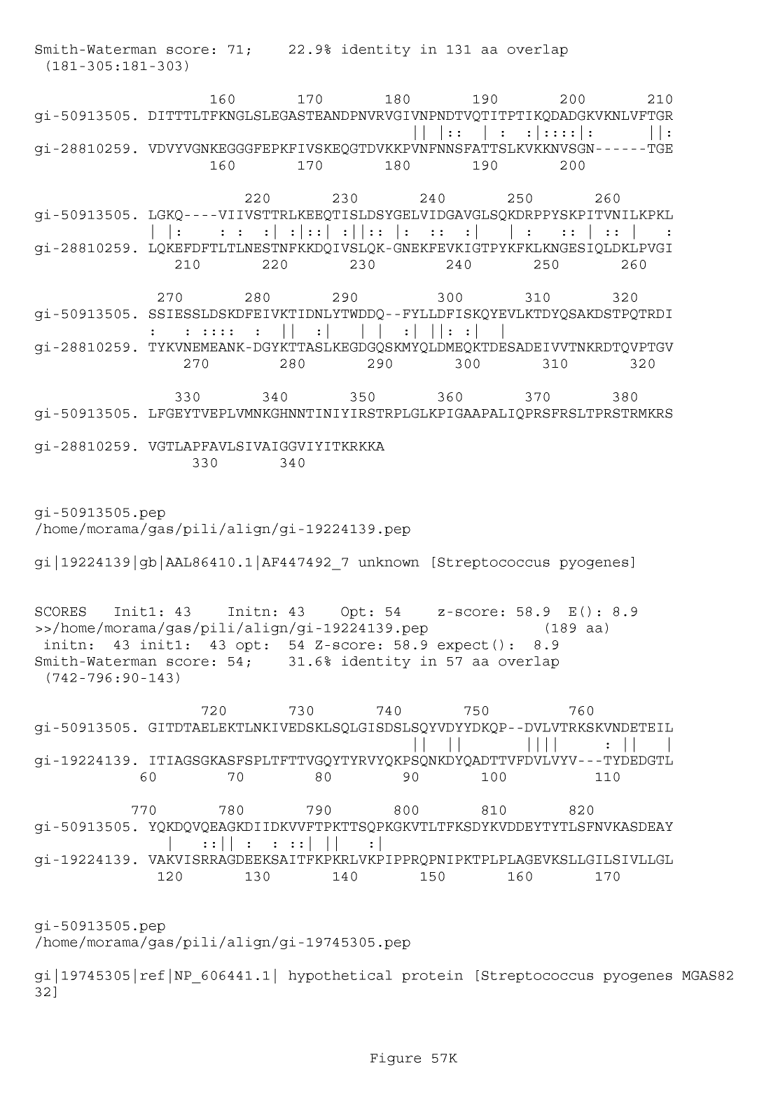

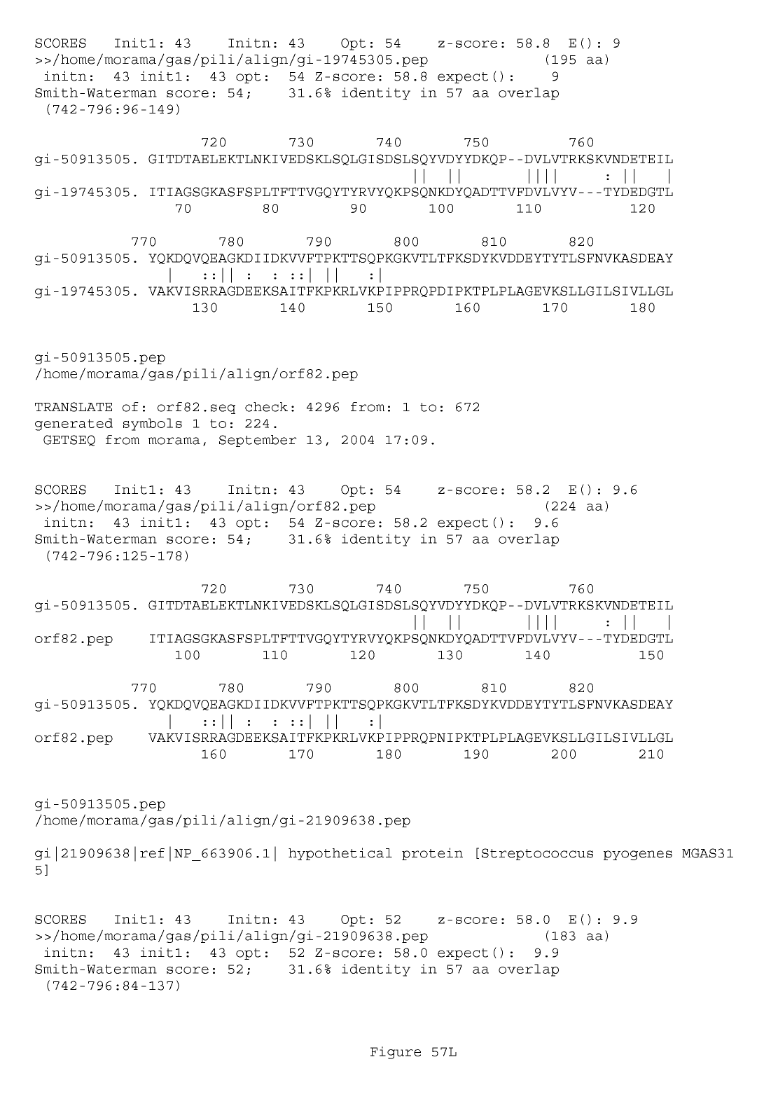

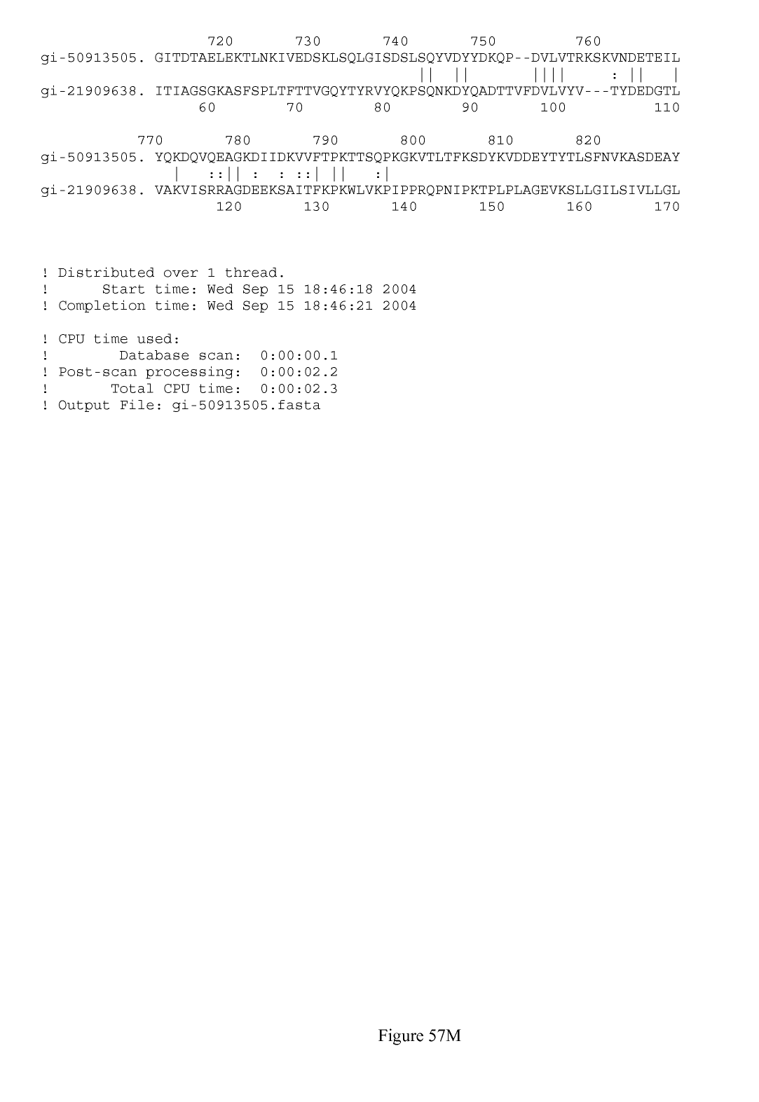

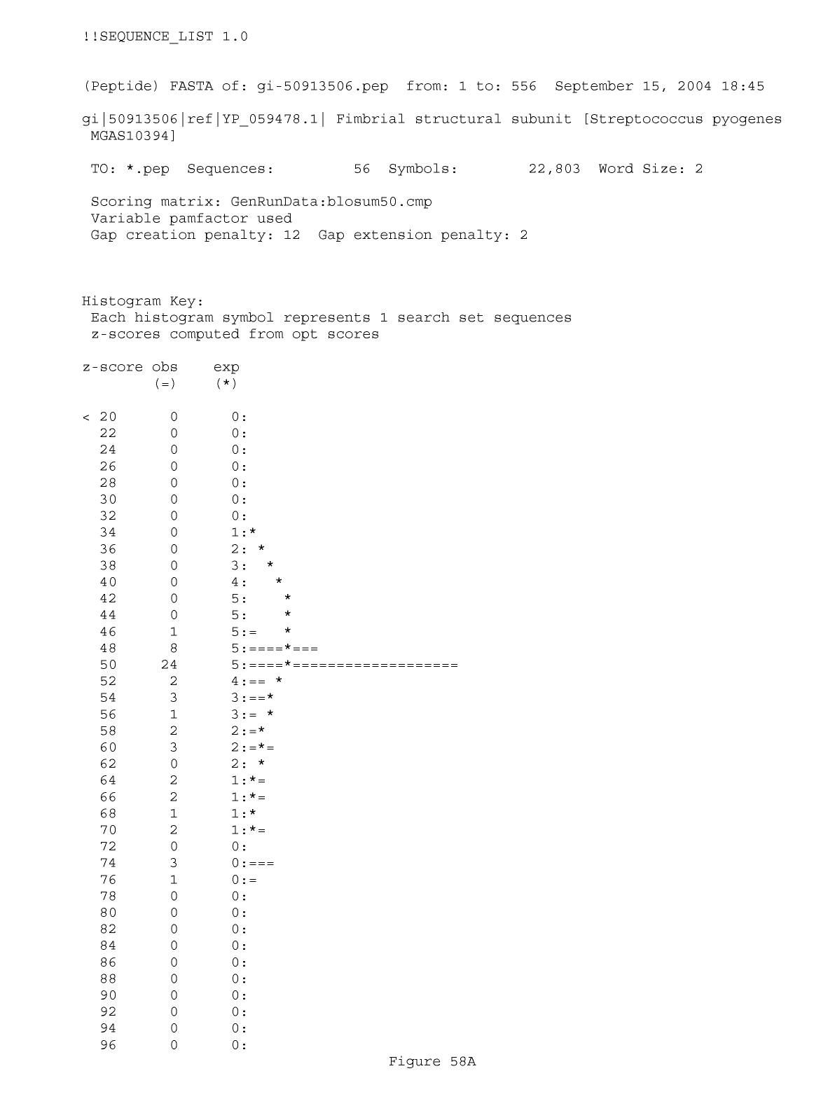

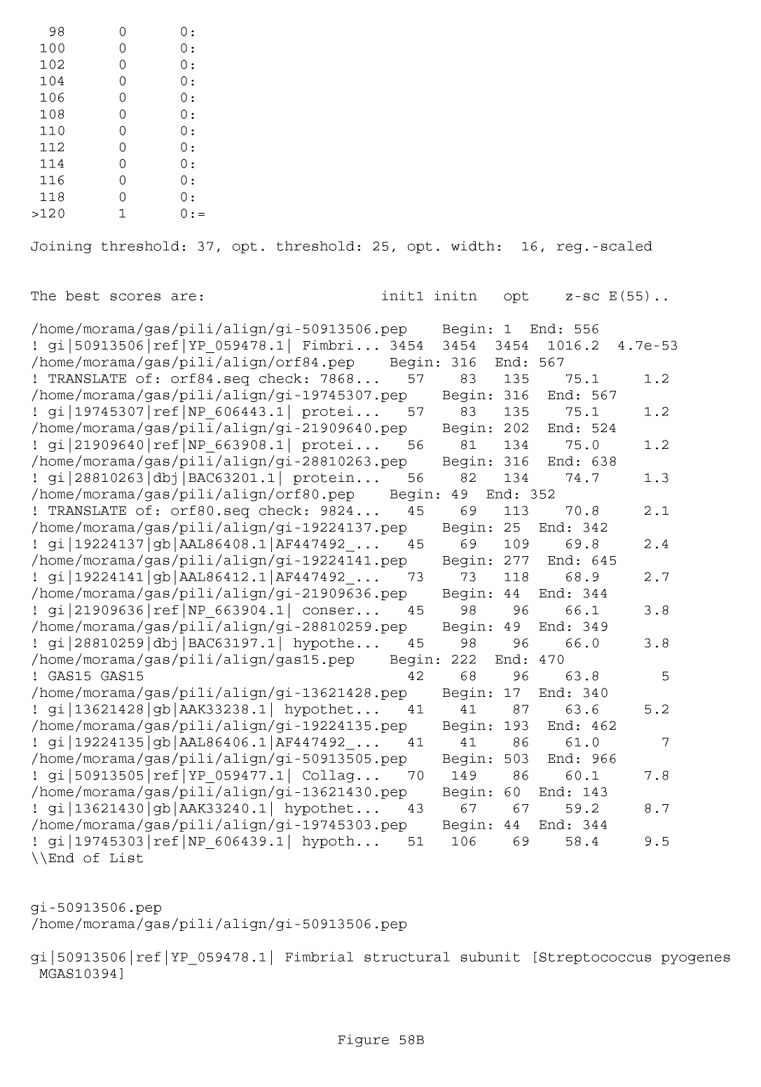

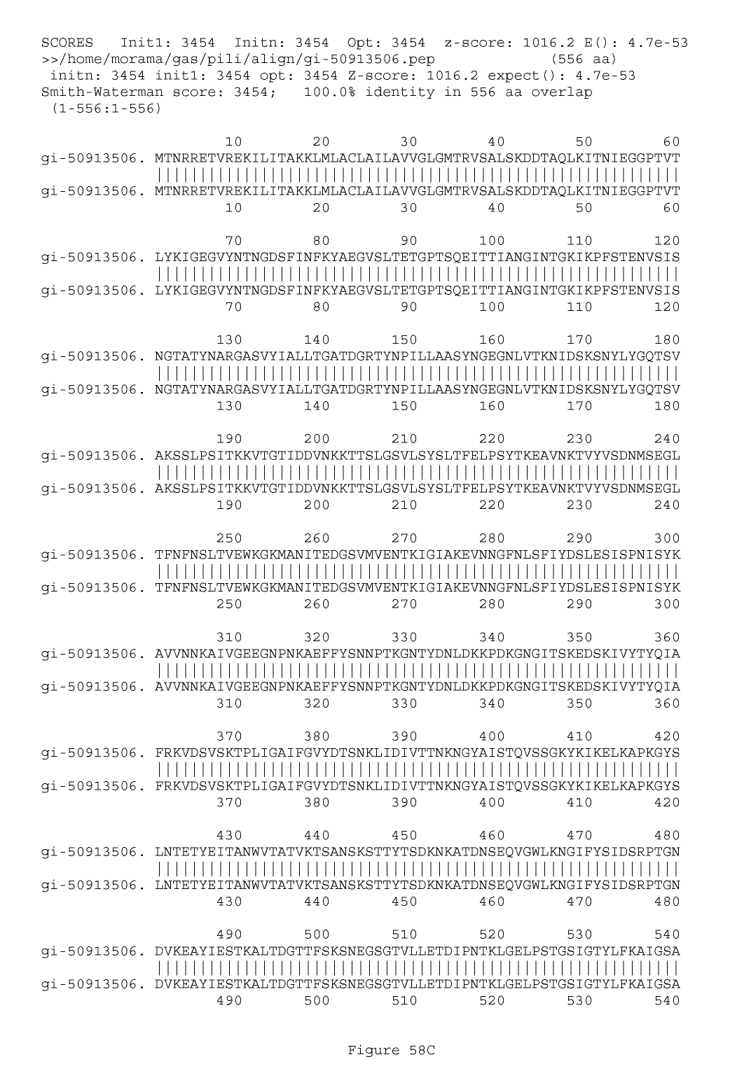

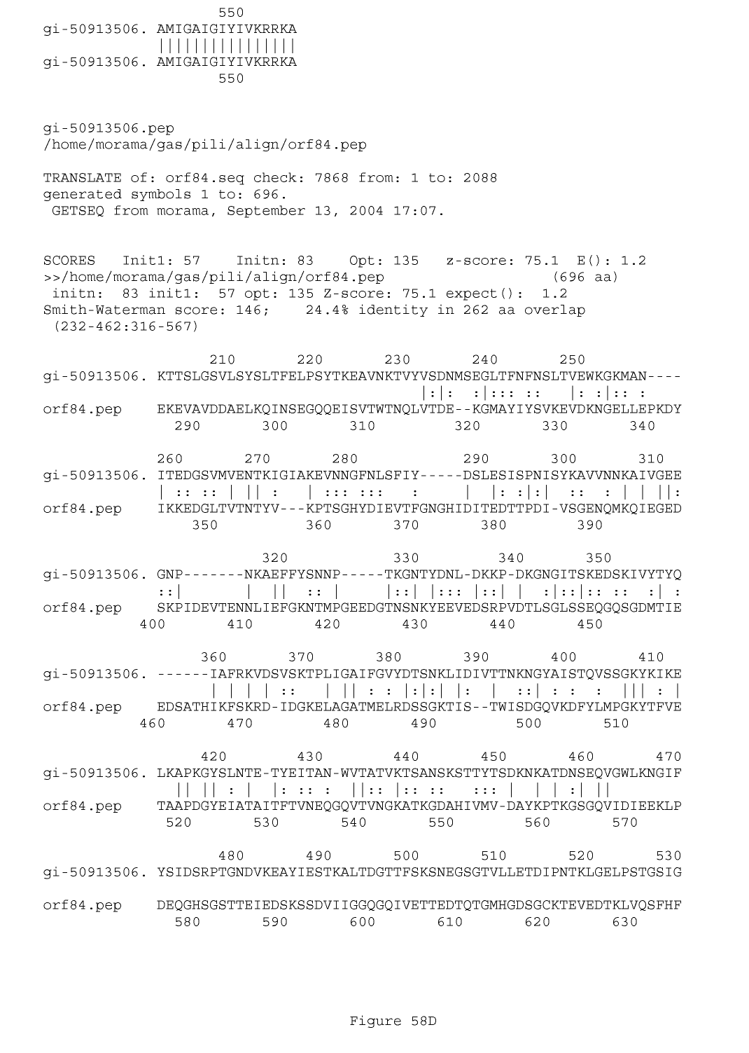

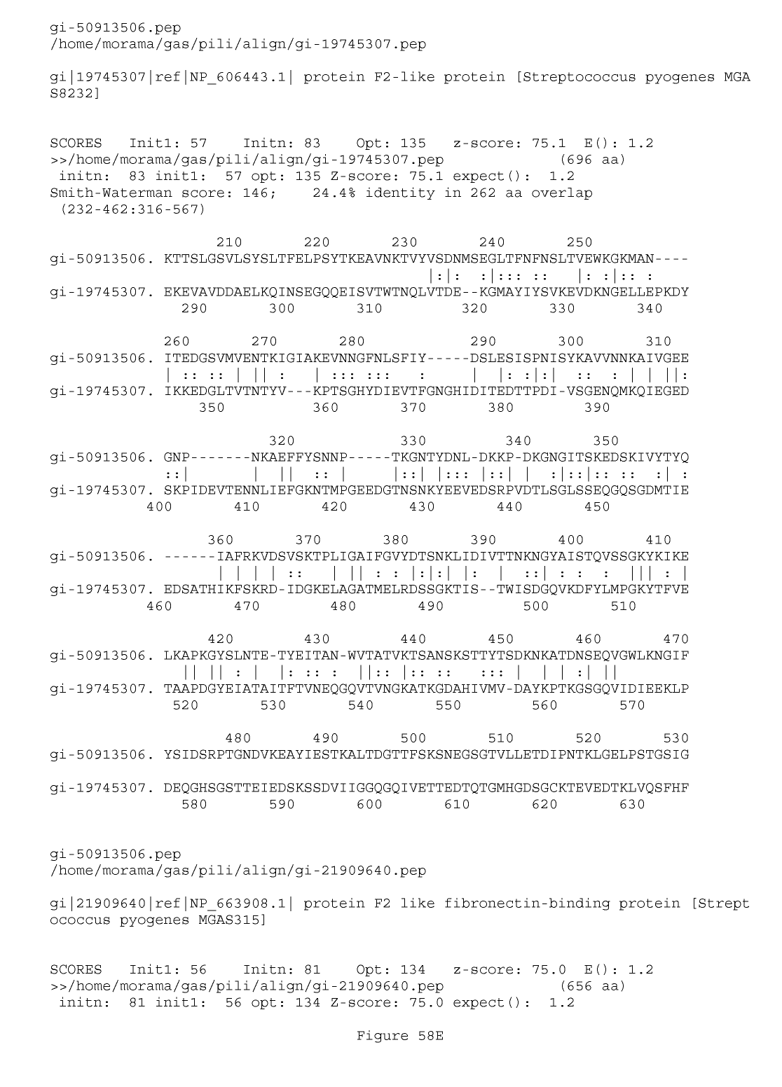

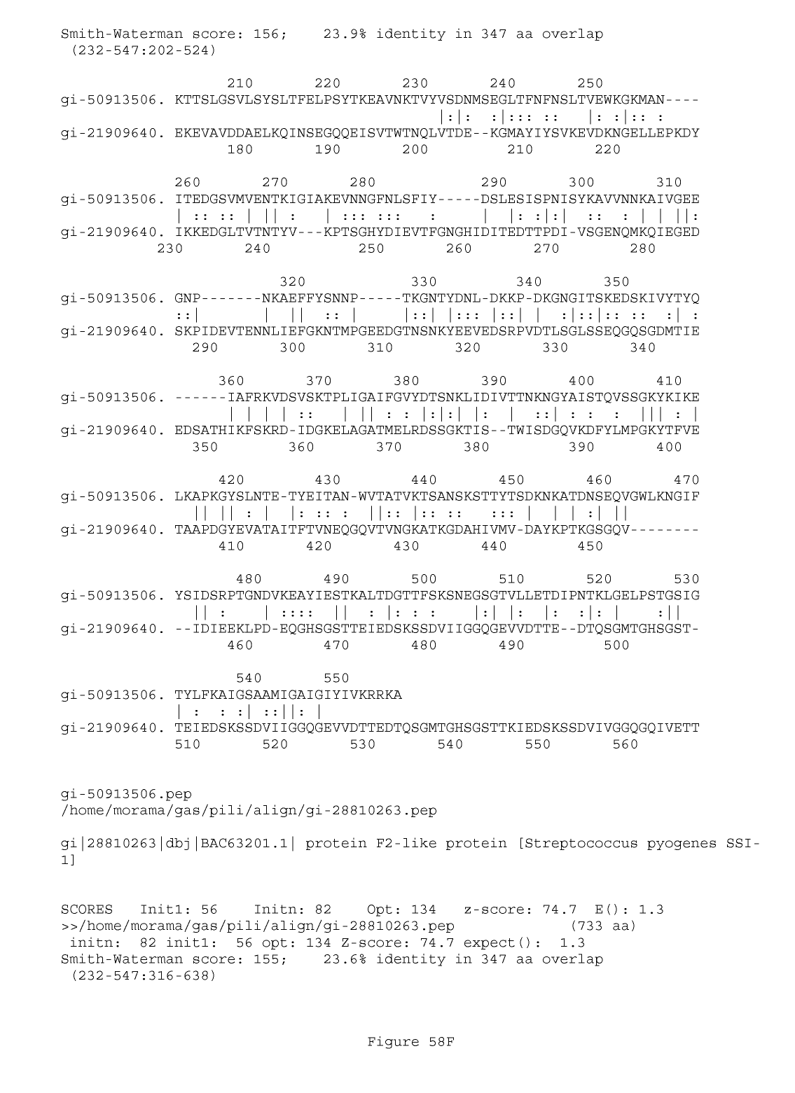

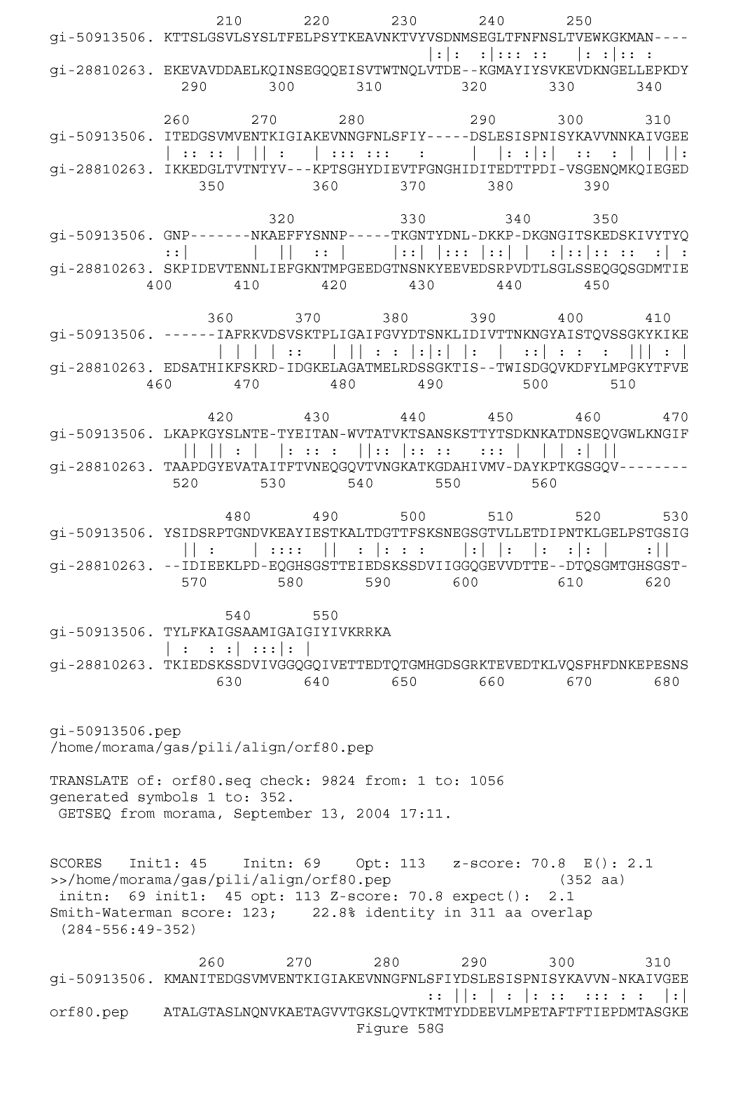

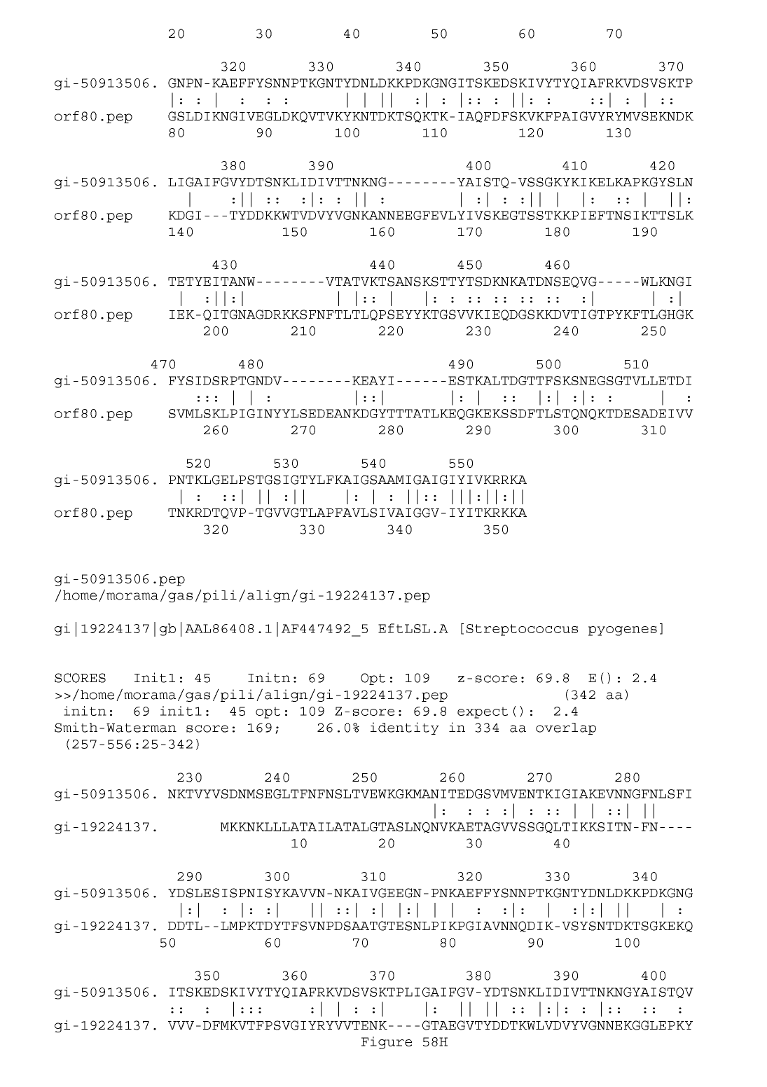

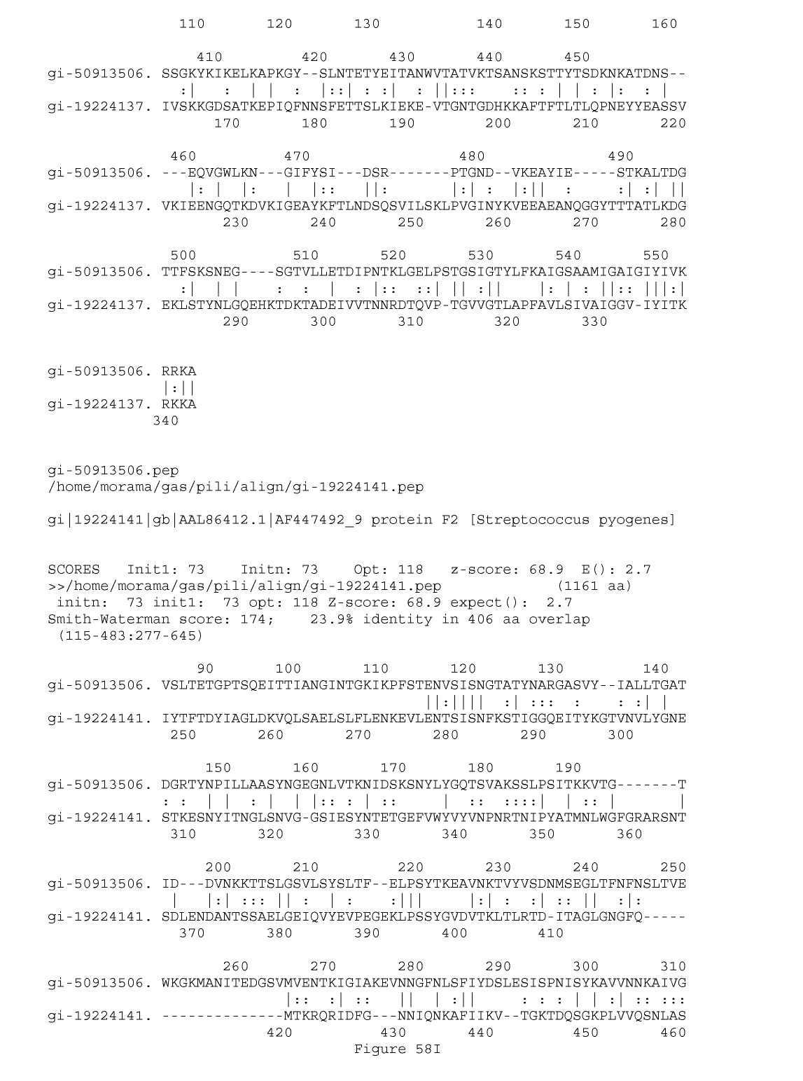

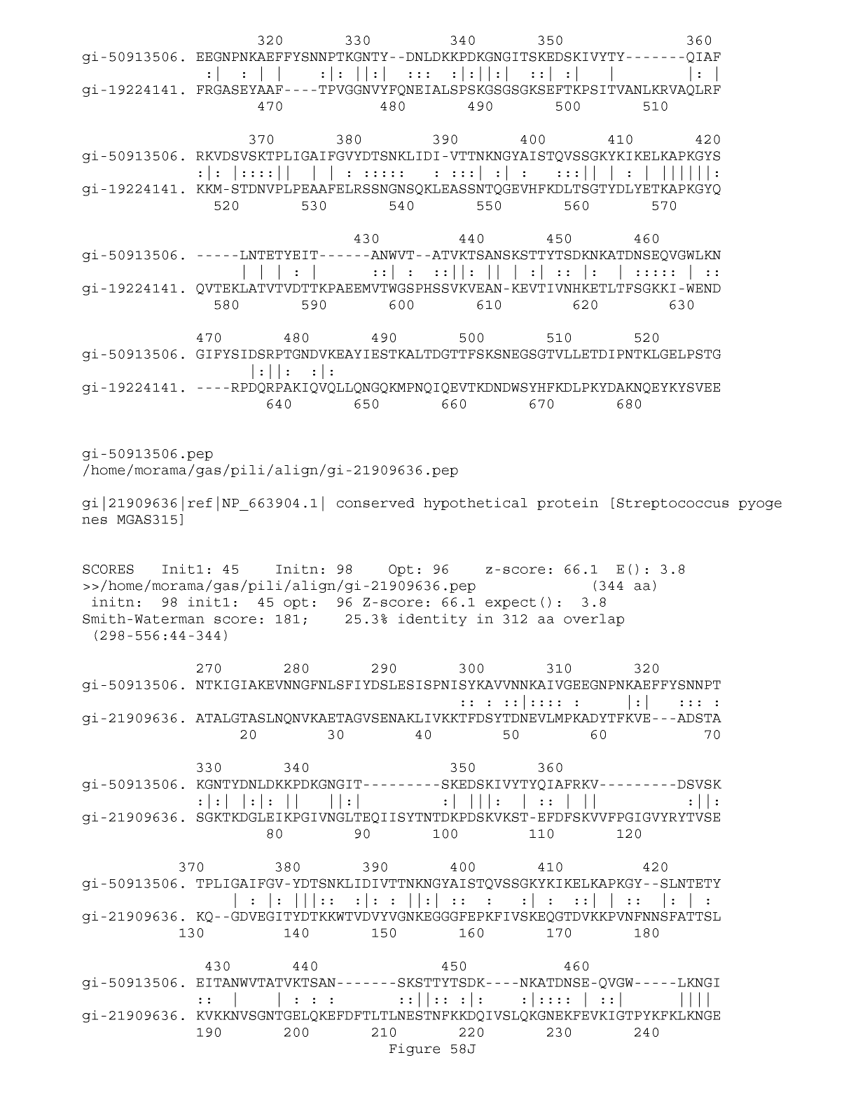

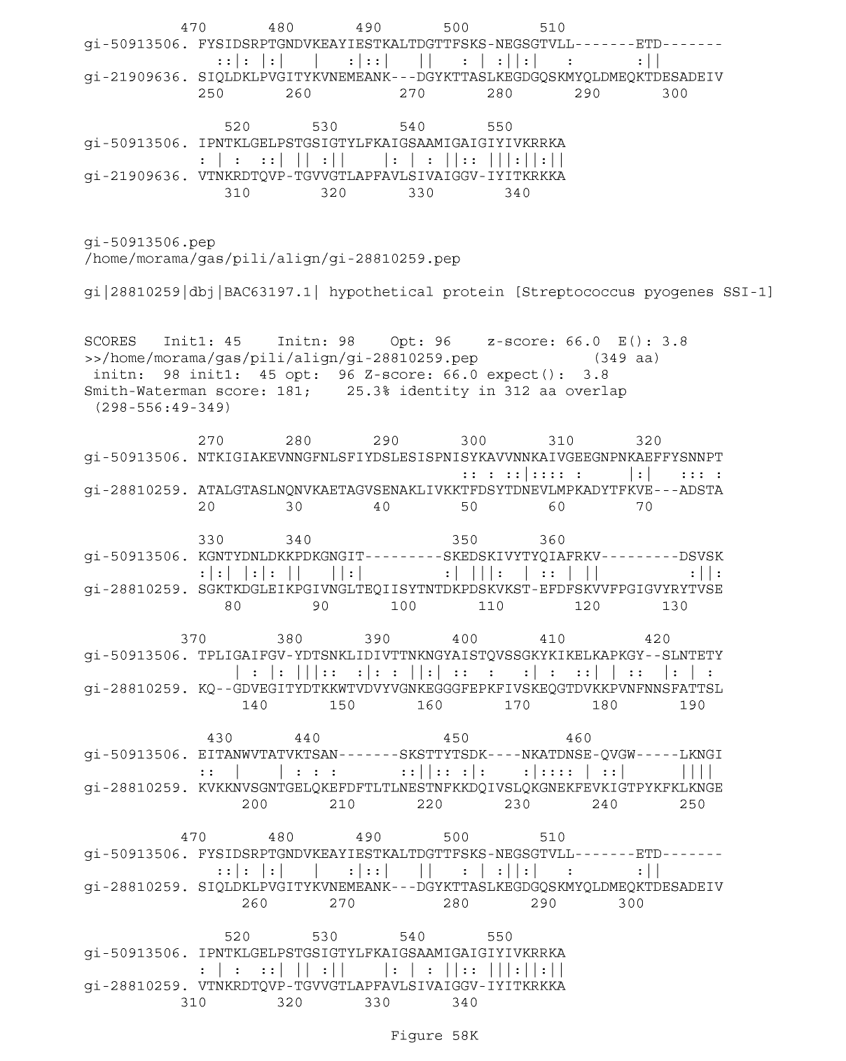

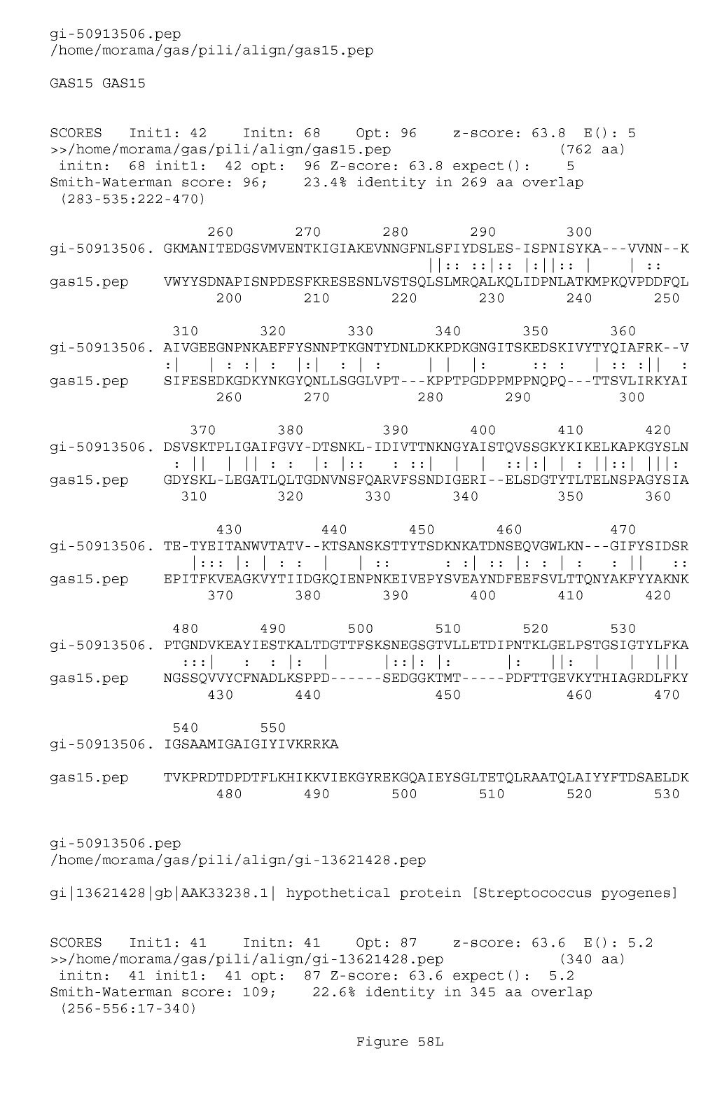

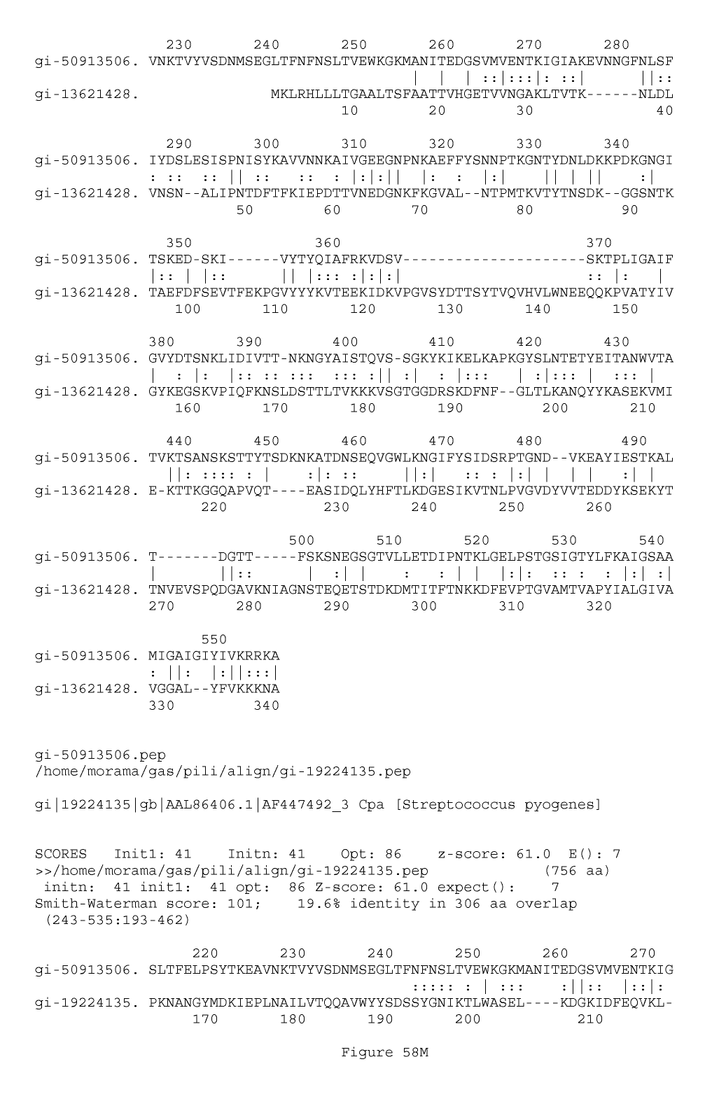

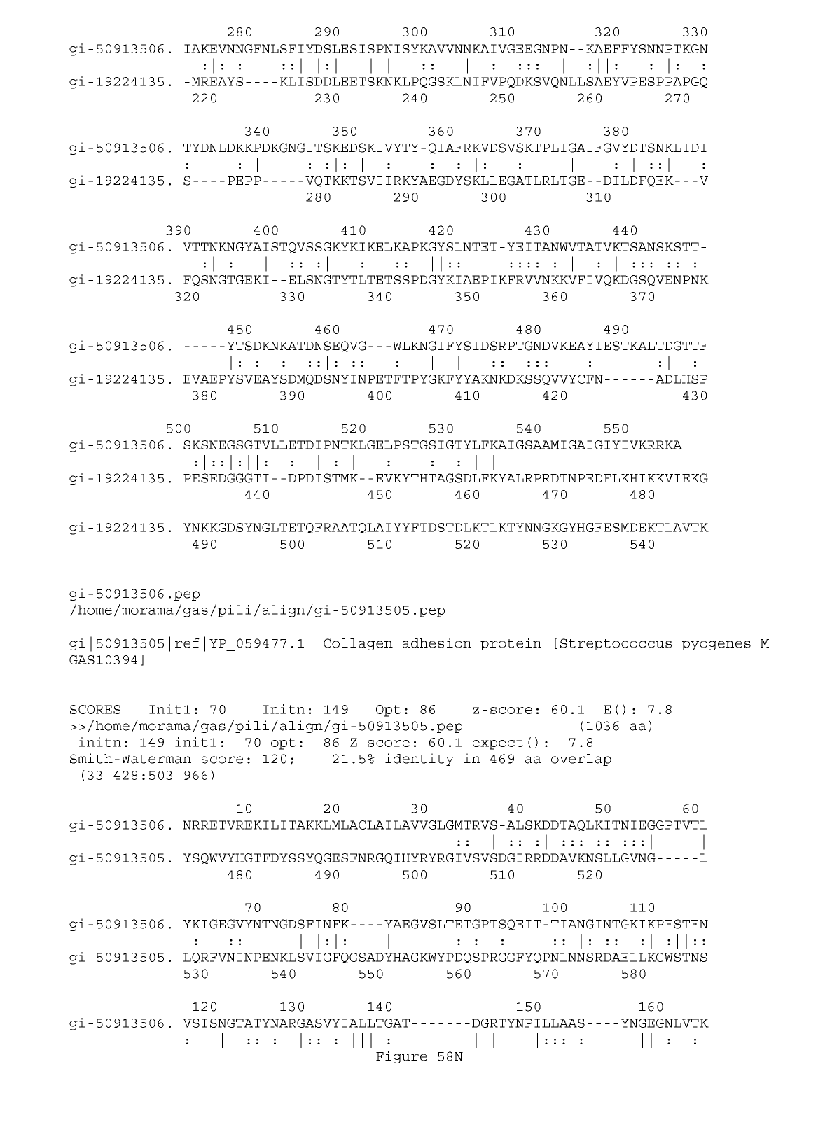

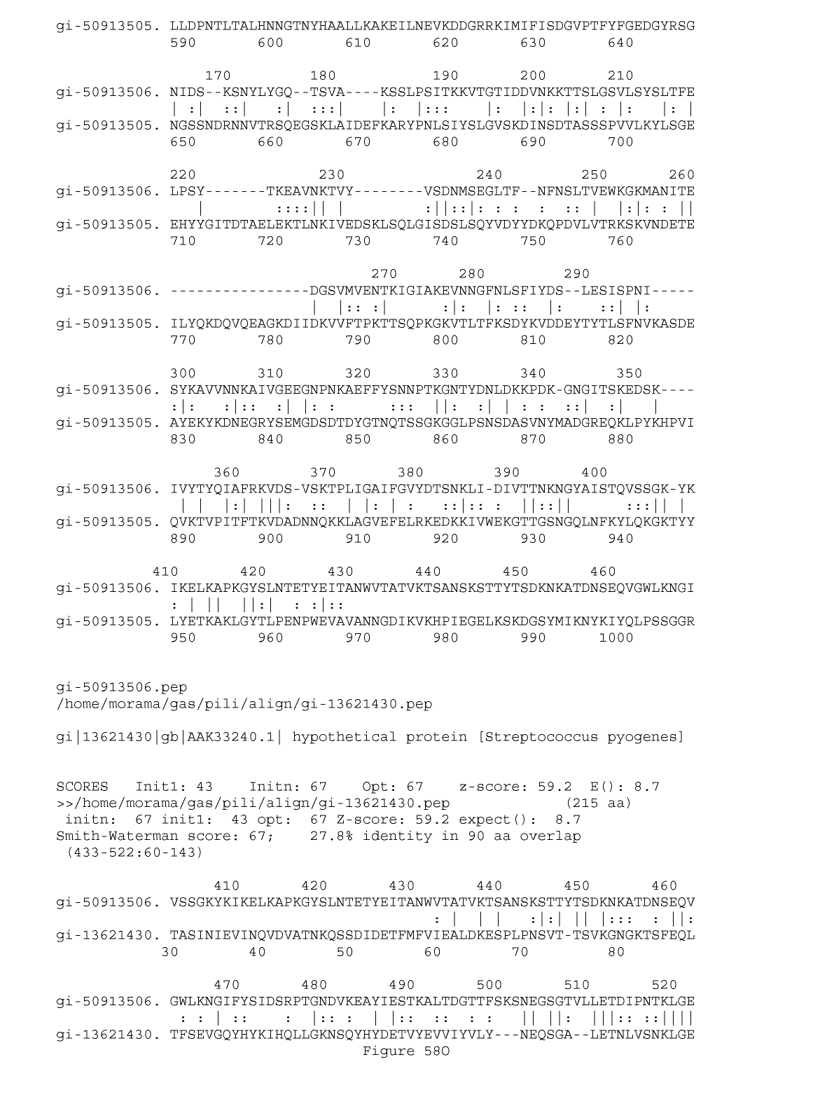

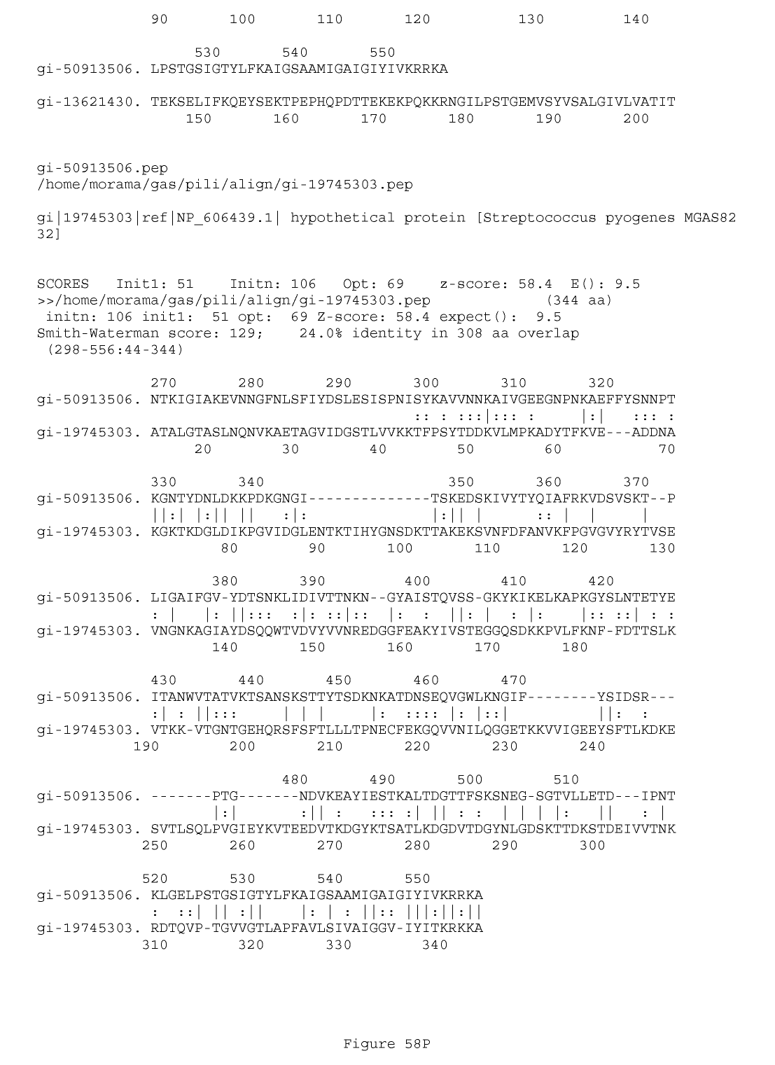

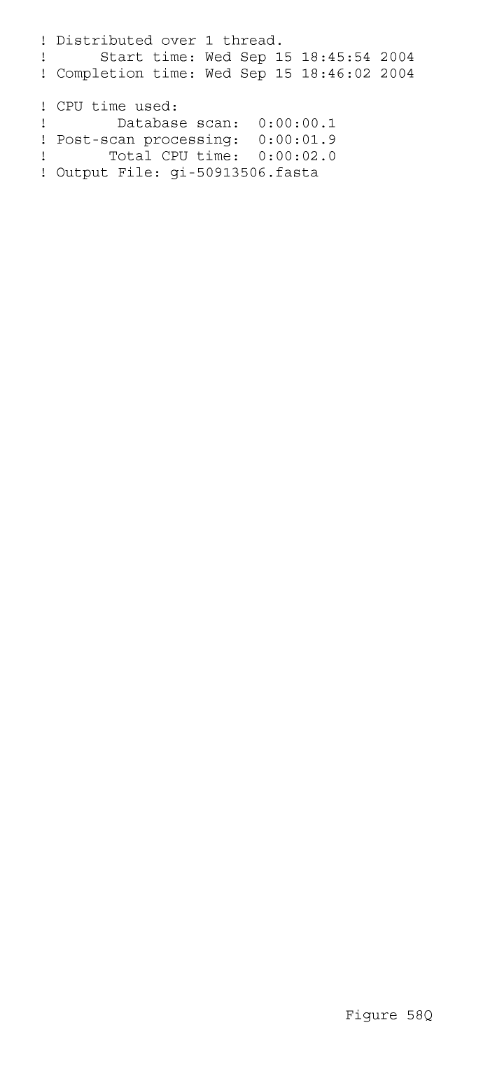

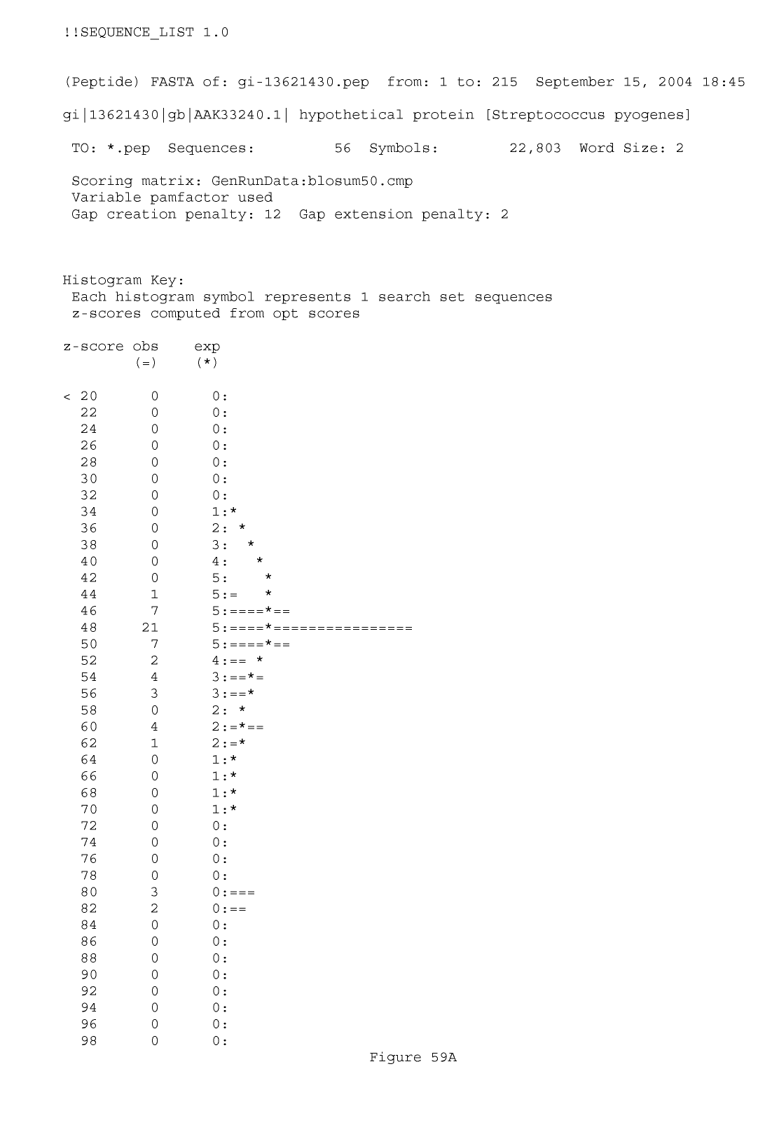

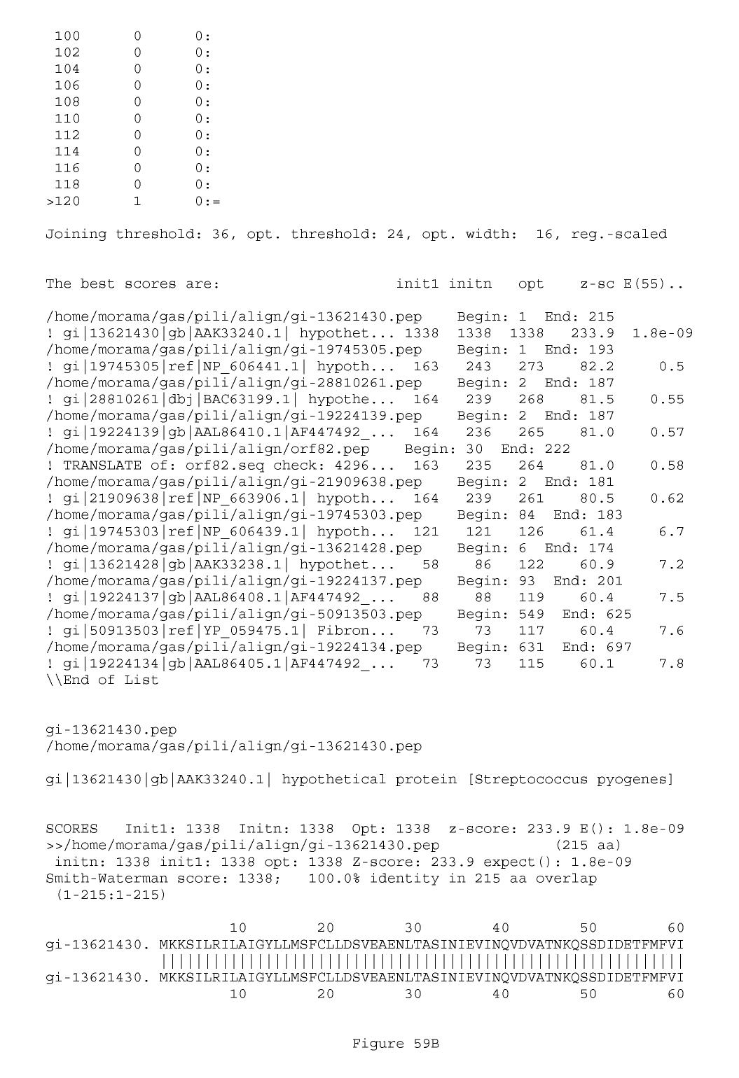

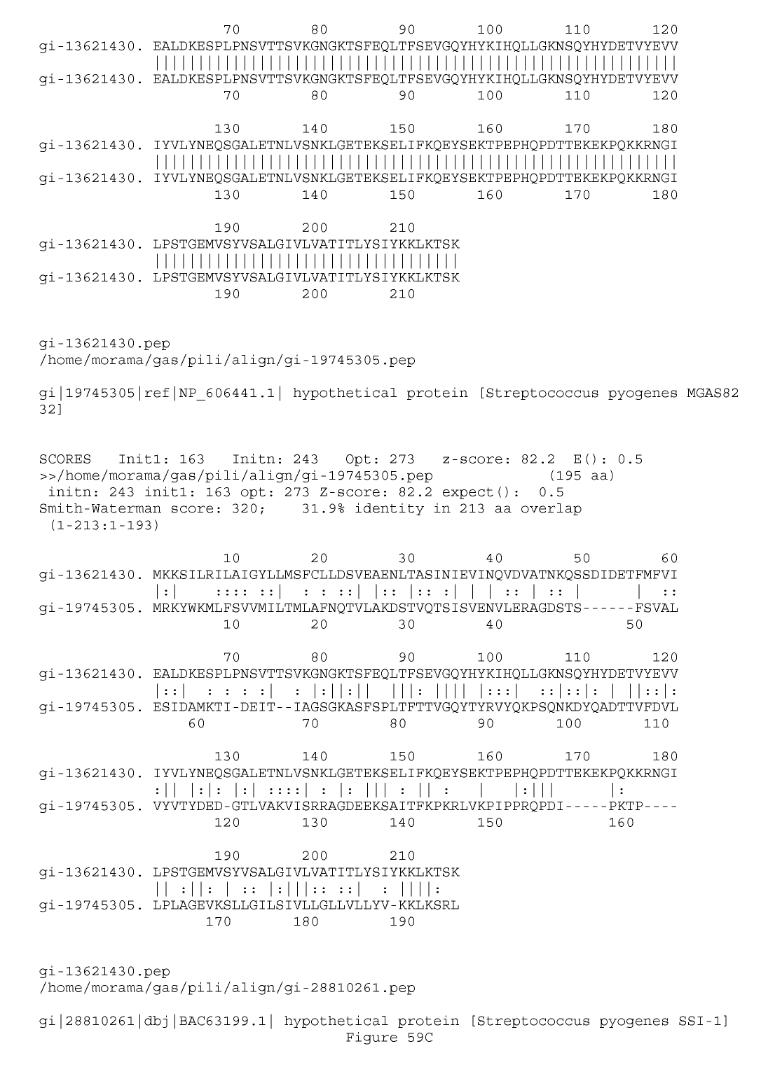

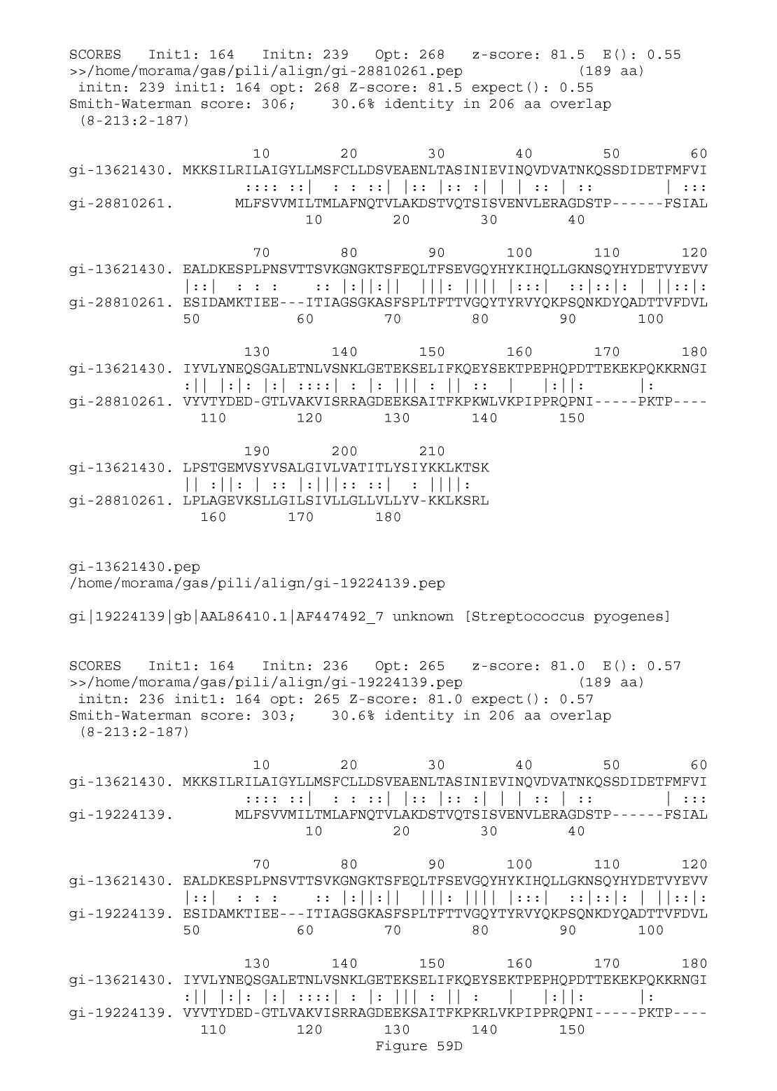

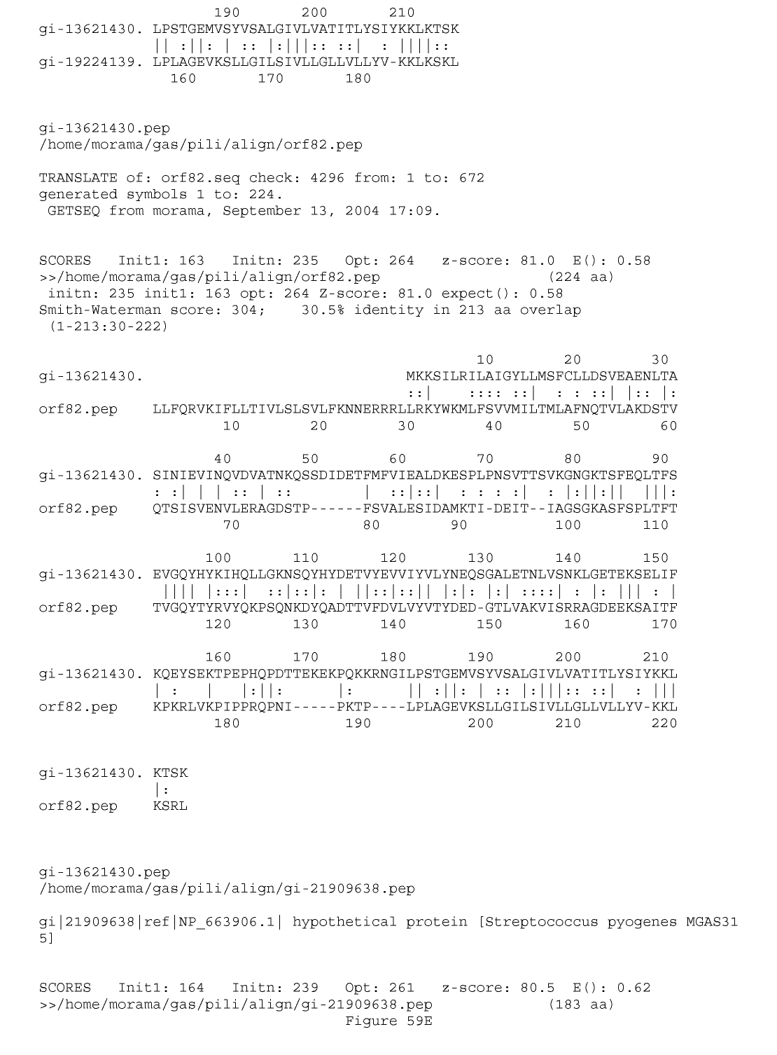

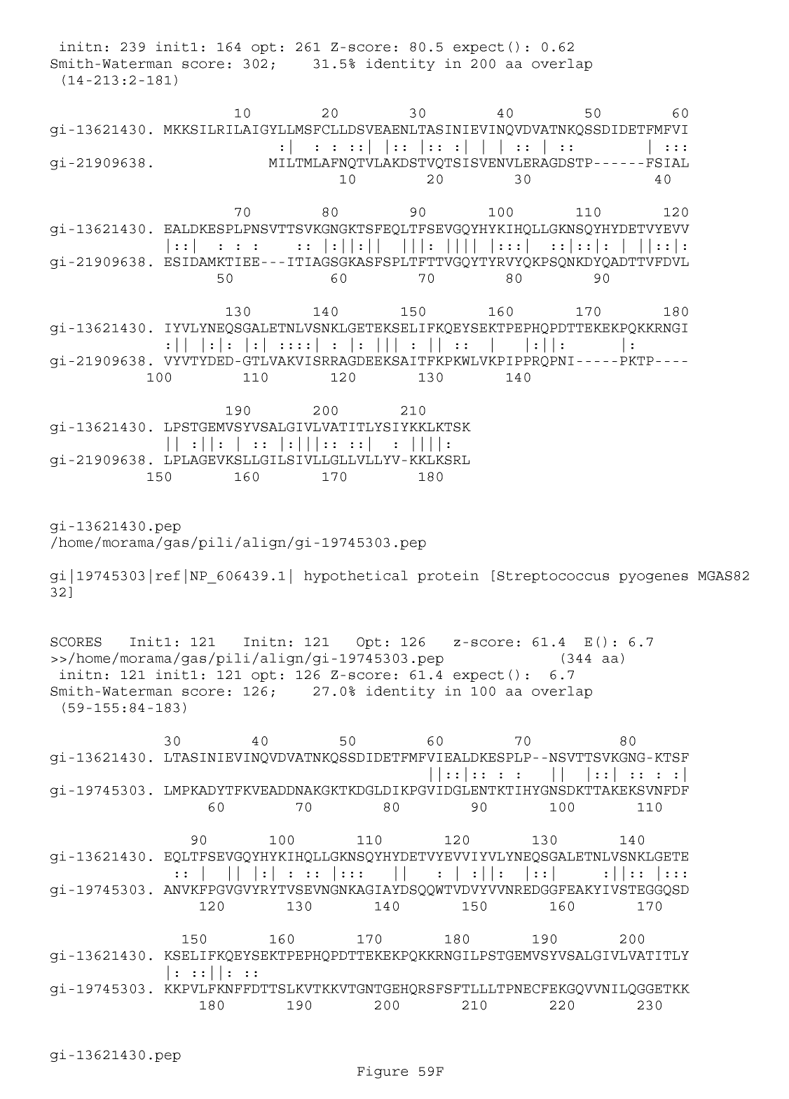

FIG. 55 is an amino acid sequence alignment that illustrates that the fimbrial protein of AI-4 serotype M12 (19224137) shares homology with the fimbrial protein of GAS AI-3 serotype M5 (ORF80), and the hypothetical protein of S. pyogenes strain MGAS315 serotype M3 (21909636), S. pyogenes strain SSI serotype M3 (28810259), S. pyogenes strain MGAS8732 serotype M3 (19745303), and S. pyogenes strain M1 GAS serotype M1 (13621428).

FIG. 56 is an amino acid sequence alignment that illustrates that the hypothetical protein of GAS AI-4 serotype M12 (19224139) shares homology with the hypothetical protein of S. pyogenes strain MGAS315 serotype M3 (21909638), S. pyogenes strain SSI-1 serotype M3 (28810261), GAS AI-3 serotype M5 (ORF82), and S. pyogenes strain MGAS8232 serotype M3 (19745305).

The protein F2-like fibronectin-binding protein of the type 4 adhesin island also contains a highly conserved pilin motif and an E-box. FIG. 60 indicates the amino acid sequence of the pilin motif and E-box in AI-4 serotype M12.

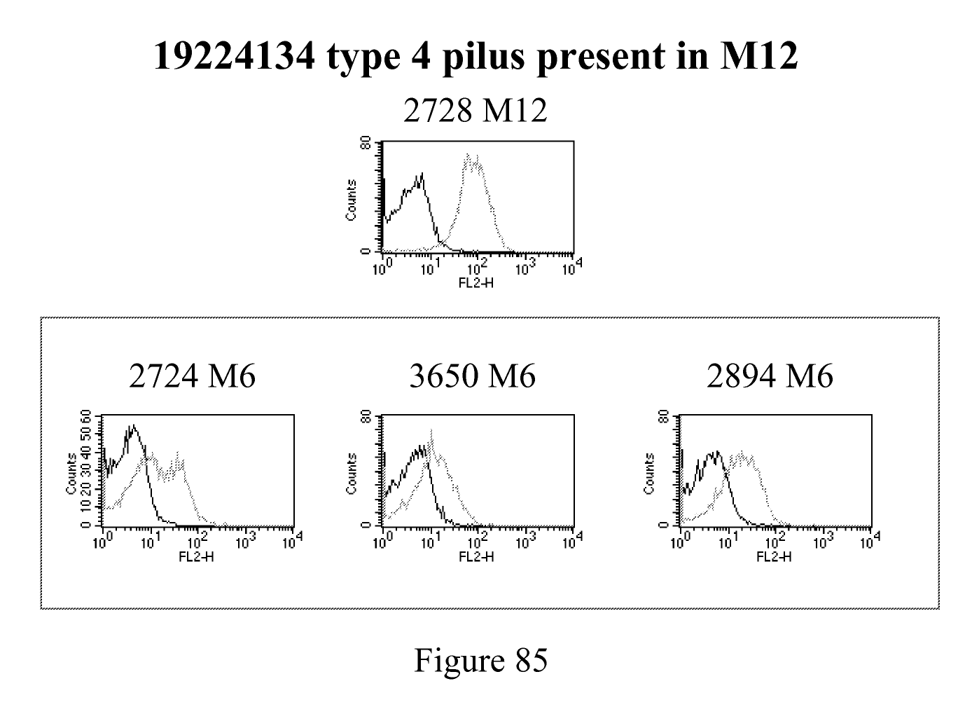

FACS analysis has confirmed that the GAS AI-4 surface proteins 19224134, 19224135, 19224137, and 19224141 are expressed on the surface of GAS. FIG. 85 provides the results of FACS analysis for surface expression of 19224134 on GAS serotype M12 2728. A shift in fluorescence is observed when anti-19224134 antiserum is present, demonstrating cell surface expression. Table 33, below, quantitatively summarizes the FACS fluorescence values obtained for GAS serotype M12 2728 in the presence of pre-immune antiserum, anti-19224134 antiserum, and the difference in fluorescence value between the pre-immune and anti-19224134 antiserum.

TABLE 33
Summary of FACS values for surface expression
of 19224134 in an M12 serotype
2728

| Pre-immune | Anti-19224134 | Change |
|---|---|---|
| 137.8 | 485.32 | 348 |

FIG. 85 also provides the results of FACS analysis for surface expression of a pilin antigen that has homology to 19224134 identified in a different GAS serotype, M6. FACS analysis conducted with the 19224134 antisera was able to detect surface expression of the homologous 19224134 antigen on each of GAS serotypes M6 2724, M6 3650, and M6 2894. Table 34, below, quantitatively summarizes the FACS fluorescence values obtained for each GAS serotype in the presence of pre-immune antiserum, anti-19224134 antiserum, and the difference in fluorescence value between the pre-immune and anti-19224134 antiserum.

TABLE 34
Summary of FACS values for surface expression of 19224134 in M6 serotypes

| 2724 | | | 3650 | | | 2894 | | |
|---|---|---|---|---|---|---|---|---|
| Pre- | Anti- | | Pre- | Anti- | | Pre- | Anti- | |
| immune | 19224134 | Change | immune | 19224134 | Change | immune | 19224134 | Change |
| 123.58 | 264.59 | 141 | 140.82 | 262.64 | 122 | 135.4 | 307.25 | 172 |

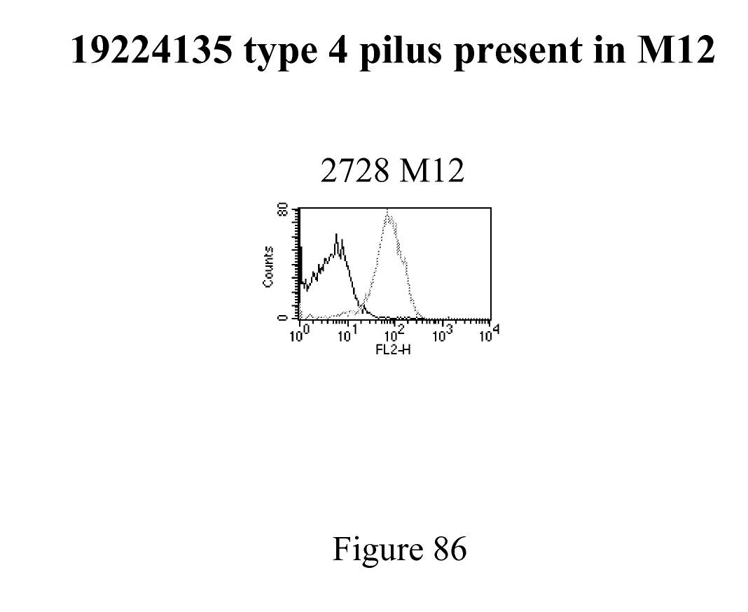

FIG. 86 provides the results of FACS analysis for surface expression of 19224135 on GAS serotype M12 2728. A shift in fluorescence is observed when anti-19224135 antiserum is present, demonstrating cell surface expression. Table 35, below, quantitatively summarizes the FACS fluorescence values obtained for GAS serotype M12 2728 in the presence of pre-immune antiserum, anti-19224135 antiserum, and the difference in fluorescence value between the pre-immune and anti-19224135 antiserum.

TABLE 35
Summary of FACS values for surface expression
of 19224135 in an M12 serotype
2728

| Pre-immune | Anti-19224135 | Change |
|---|---|---|
| 151.38 | 471.95 | 321 |

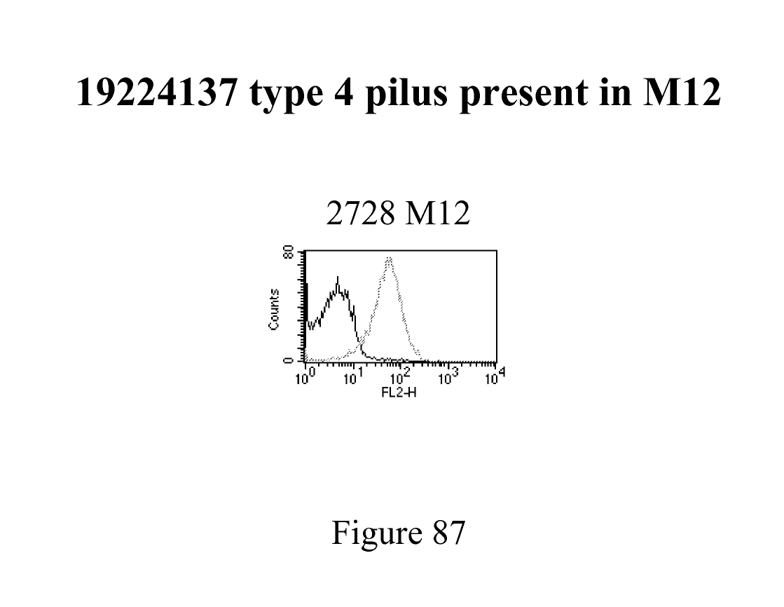

FIG. 87 provides the results of FACS analysis for surface expression of 19224137 on GAS serotype M12 2728. A shift in fluorescence is observed when anti-19224137 antiserum is present, demonstrating cell surface expression. Table 36, below, quantitatively summarizes the FACS fluorescence values obtained for GAS serotype M12 2728 in the presence of pre-immune antiserum, anti-19224137 antiserum, and the difference in fluorescence value between the pre-immune and anti-19224137 antiserum.

TABLE 36
Summary of FACS values for surface expression
of 19224137 in an M12 serotype
2728

| Pre-immune | Anti-19224137 | Change |
|---|---|---|
| 140.44 | 433.25 | 293 |

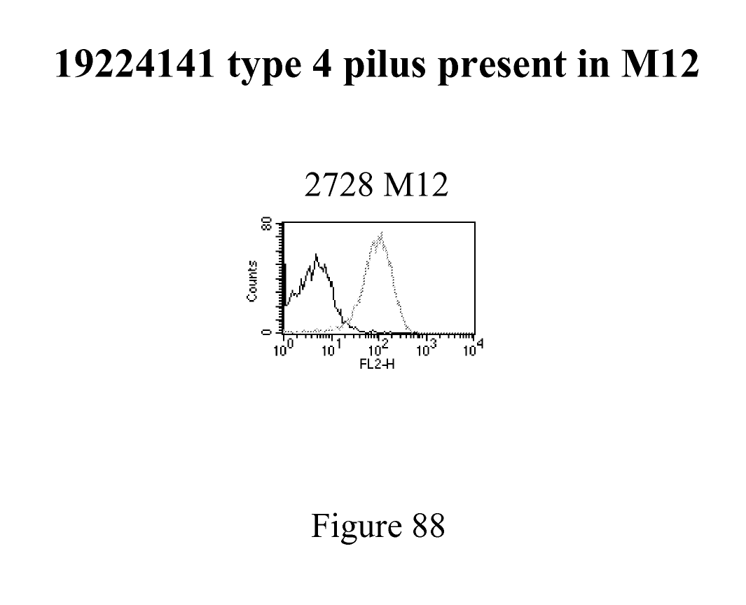

FIG. 88 provides the results of FACS analysis for surface expression of 19224141 on GAS serotype M12 2728. A shift in fluorescence is observed when anti-19224141 antiserum is present, demonstrating cell surface expression. Table 37, below, quantitatively summarizes the FACS fluorescence values obtained for GAS serotype M12 2728 in the presence of pre-immune antiserum, anti-19224141 antiserum, and the difference in fluorescence value between the pre-immune and anti-19224141 antiserum.

TABLE 37
Summary of FACS values for surface expression
of 19224141 in an M12 serotype
2728

| Pre-immune | Anti-19224141 | Change |
|---|---|---|
| 147.02 | 498 | 351 |

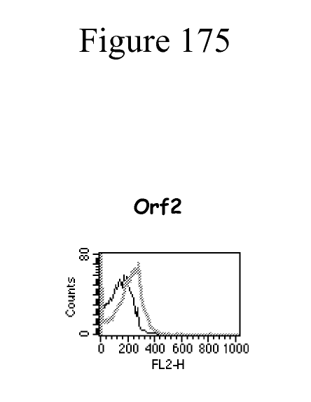

19224139 (designated as orf2) may also be expressed on the surface of GAS serotype M12 bacteria. FIG. 175 shows the results of FACS analysis for surface expression of 19224139 on M12 strain 2728. A slight shift in fluorescence is observed, which demonstrates that some 19224139 may be expressed on the GAS cell surface.

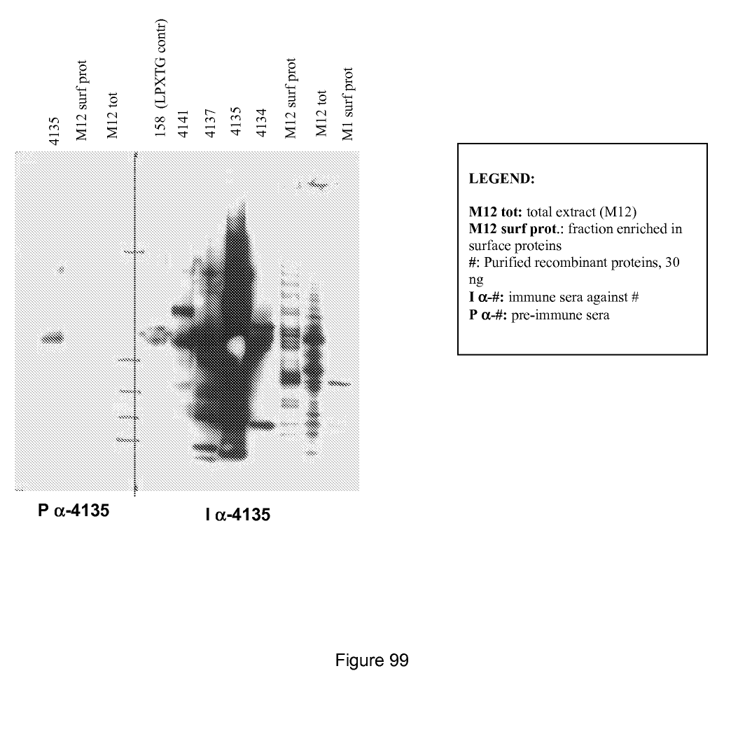

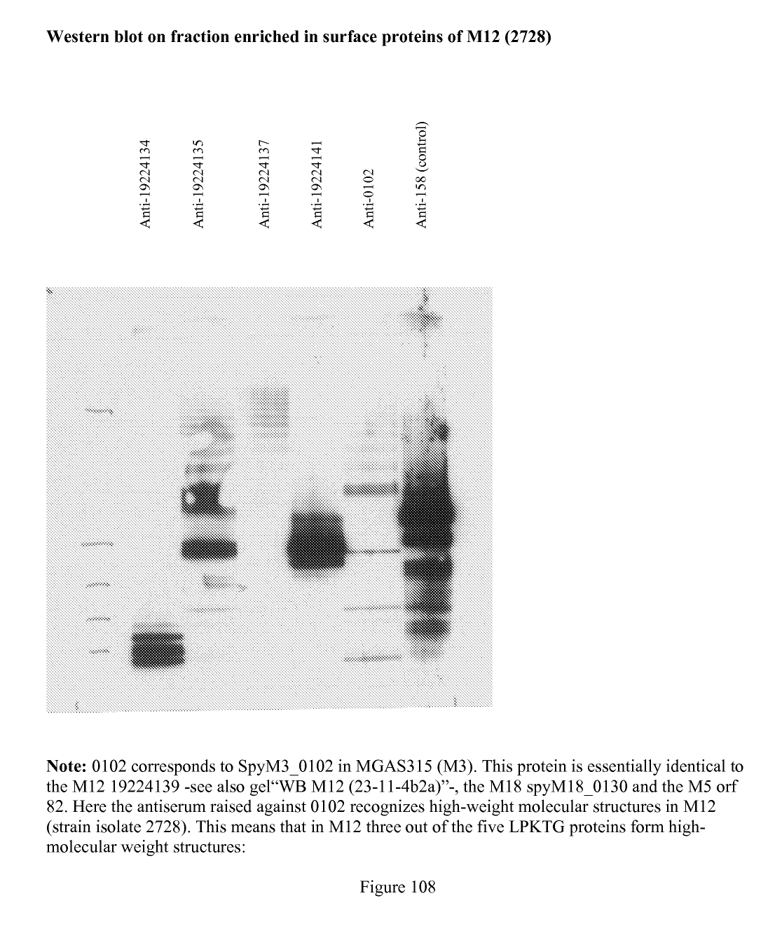

Surface expression of 19224135 on M12 serotype GAS has also been confirmed by Western blot analysis. FIG. 99 shows that while pre-immune sera (P α-4135) does not detect GAS M12 expression of 19224135, anti-19224135 immune sera (I α-4135) is able to detect 19224135 protein in both total GAS M12 extracts (M12 tot) and GAS M12 fractions enriched for cell surface proteins (M12 surf prot). The 19224135 proteins detected in the total GAS M12 extracts or the GAS M12 extracts enriched for surface proteins are also present as high molecular weight structures, indicating that 19224135 may be in an oligomeric (pilus) form. See also FIG. 108, which provides a further Western blot showing that anti-19224135 antiserum (Anti-19224135) immunoreacts with high molecular weight structures in GAS M12 strain 2728 protein extracts enriched for surface proteins.

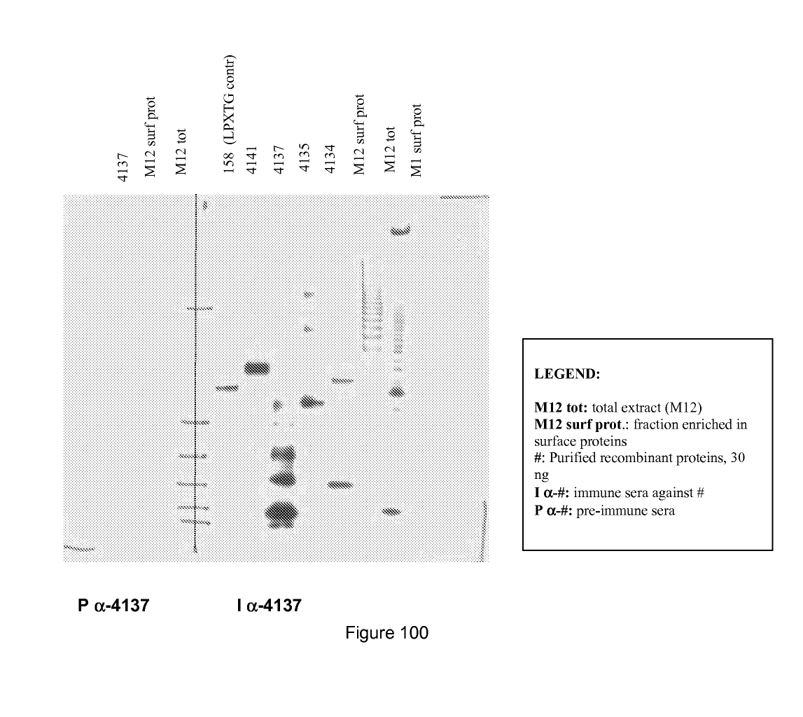

Surface expression of 19224137 on M12 serotype GAS has also been confirmed by Western blot analysis. FIG. 100 shows that while pre-immune sera (P α-4137) does not detect GAS M12 expression of 19224137, anti-19224137 immune sera (I α-4137) is able to detect 19224137 protein in both total GAS M12 extracts (M12 tot) and GAS M12 fractions enriched for cell surface proteins (M12 surf prot). The 19224137 proteins detected in the total GAS M12 extracts or the GAS M12 extracts enriched for surface proteins are also present as high molecular weight structures, indicating that 19224137 may be in an oligomeric (pilus) form. See also FIG. 108, which provides a further Western blot showing that anti-19224137 antiserum (Anti-19224137) immunoreacts with high molecular weight structures in GAS M12 strain 2728 protein extracts enriched for surface proteins.

Streptococcus pneumoniae

Adhesin island sequences can be identified in Streptococcus pneumoniae genomes. Several of these genomes include the publicly available Streptococcus pneumoniae TIGR4 genome or Streptococcus pneumoniae strain 670 genome. Examples of these S. pneumoniae AI sequence are discussed below.

S. pneumoniae Adhesin Islands generally include a series of open reading frames within a S. pneumoniae genome that encode for a collection of surface proteins and sortases. A S. pneumoniae Adhesin Island may encode for amino acid sequences comprising at least one surface protein. Alternatively, an S. pneumoniae Adhesin Island may encode for at least two surface proteins and at least one sortase. Preferably, a S. pneumoniae Adhesin Island encodes for at least three surface proteins and at least two sortases. One or more of the surface proteins may include an LPXTG motif (e.g., SEQ ID NO:122) or other sortase substrate motif. One or more S. pneumoniae AI surface proteins may participate in the formation of a pilus structure on the surface of the S. pneumoniae bacteria.

S. pneumoniae Adhesin Islands of the invention preferably include a divergently transcribed transcriptional regulator. The transcriptional regulator may regulate the expression of the S. pneumoniae AI operon.

The S. pneumoniae AI surface proteins may bind or otherwise adhere to fibrinogen, fibronectin, or collagen.

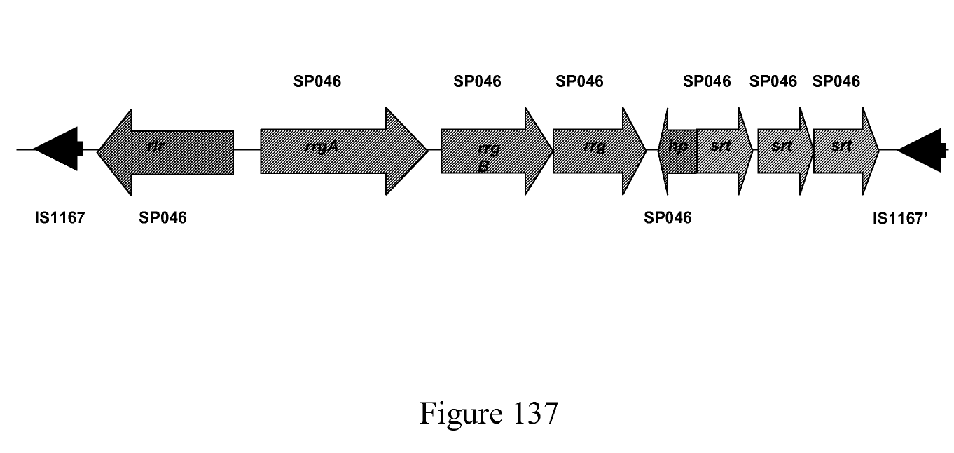

A schematic of the organization of a S. pneumoniae AI locus is provided in FIG. 137. The locus comprises open reading frames encoding a transcriptional regulator (r1rA), cell wall surface proteins (rrgA, rrgB, rrgC), and sortases (srtB, srtC, srtD). FIG. 137 also indicates the S. pneumoniae strain TIGR4 gene name corresponding to each of these open reading frames.

Tables 9 and 38 identify the genomic location of each of these open reading frames in S. pneumoniae strains TIGR4 and 670, respectively.

TABLE 9
S. pneumoniae AI sequences from TIGR4

| | | | | Synonym | |
|---|---|---|---|---|---|
| | | | | (AI Sequence | |
| Genomic Location | Strand | Length | PID | Identifier) | Functional description |
| 436302 . . . 437831 | − | 509 | 15900377 | SP0461 | transcriptional regulator |
| 438326 . . . 441007 | + | 893 | 15900378 | SP0462 | cell wall surface anchor family protein |
| 441231 . . . 443228 | + | 665 | 15900379 | SP0463 | cell wall surface anchor family protein |
| 443275 . . . 444456 | + | 393 | 15900380 | SP0464 | cell wall surface anchor family protein |
| 444675 . . . 444806 | − | 43 | 15900381 | SP0465 | hypothetical protein |
| 444857 . . . 445696 | + | 279 | 15900382 | SP0466 | sortase |
| 445791 . . . 446576 | + | 261 | 15900383 | SP0467 | sortase |
| 446563 . . . 447414 | + | 283 | 15900384 | SP0468 | sortase |

TABLE 38
S. pneumoniae strain 670 AI sequences

| Genomic | | AI Sequence | |
|---|---|---|---|
| Location | Strand | Identifier\ | Functional description |
| 4383-5645 | − | Orf1_670 | IS1167, transposase |
| 5910-7439 | − | Orf2_670 | transcriptional regulator, putative |
| 7934-10606 | + | Orf3_670 | cell wall surface anchor family |
| | | | protein |
| 10839-12773 | + | Orf4_670 | cell wall surface anchor family |
| | | | protein |
| 12796-14001 | + | Orf5_670 | cell wall surface anchor family |
| | | | protein |
| 14327-15241 | + | Orf6_670 | sortase, putative |
| 15336-16121 | + | Orf7_670 | sortase, putative |
| 16108-16959 | + | Orf8_670 | sortase, putative |

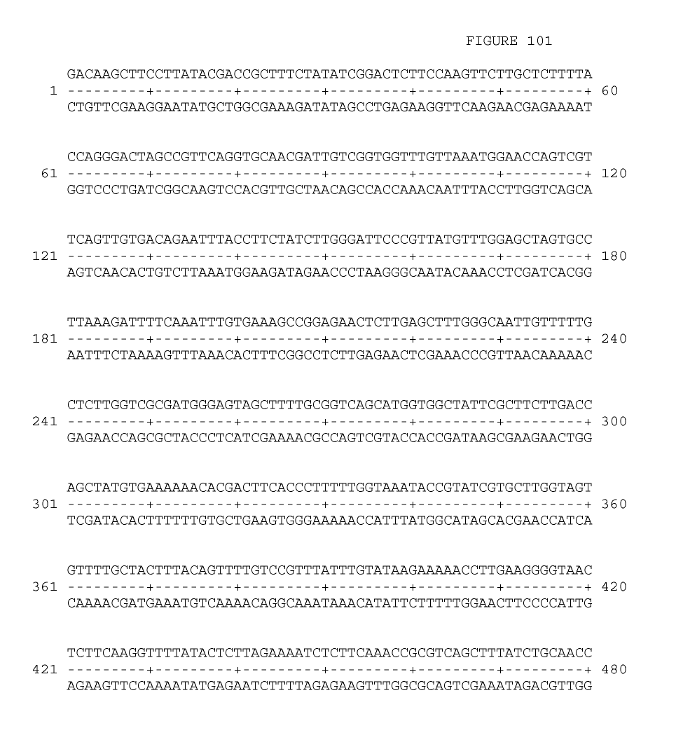

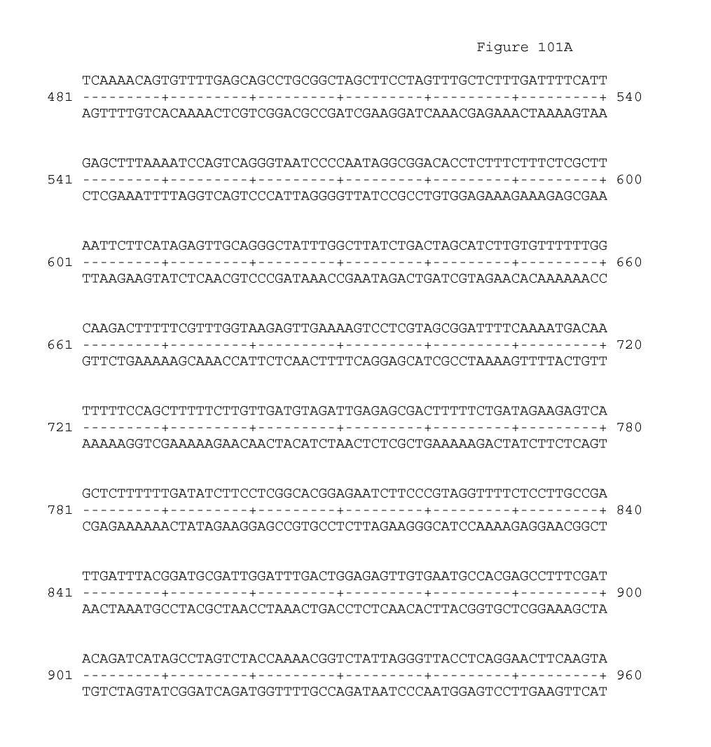

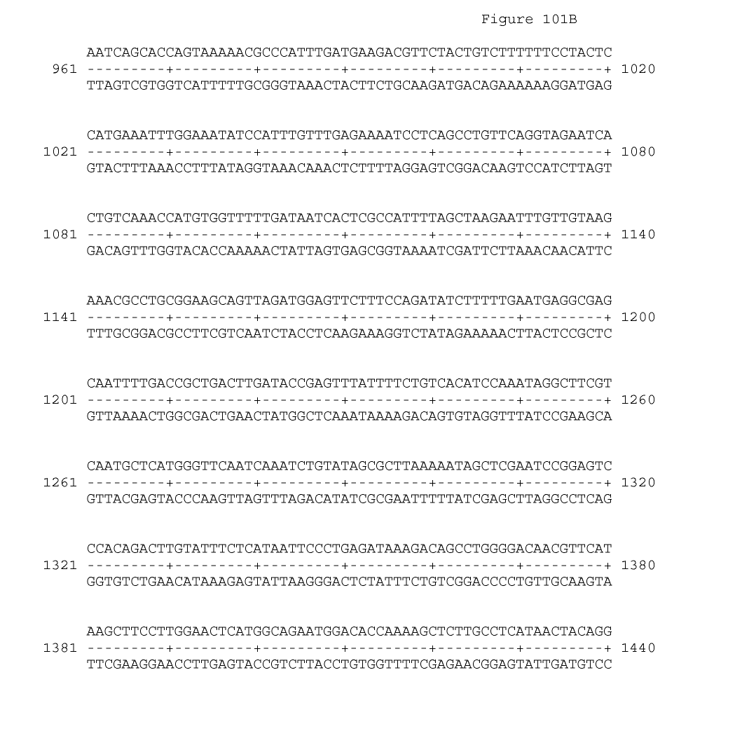

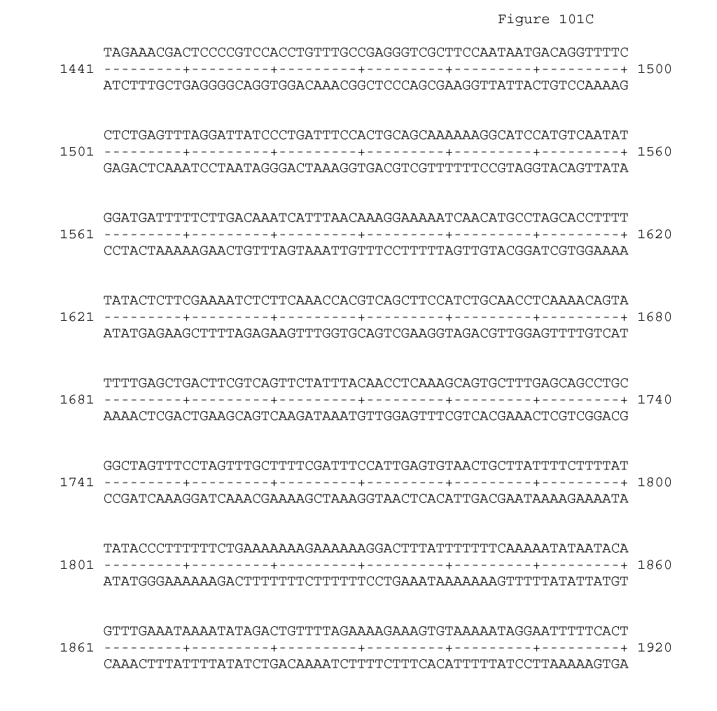

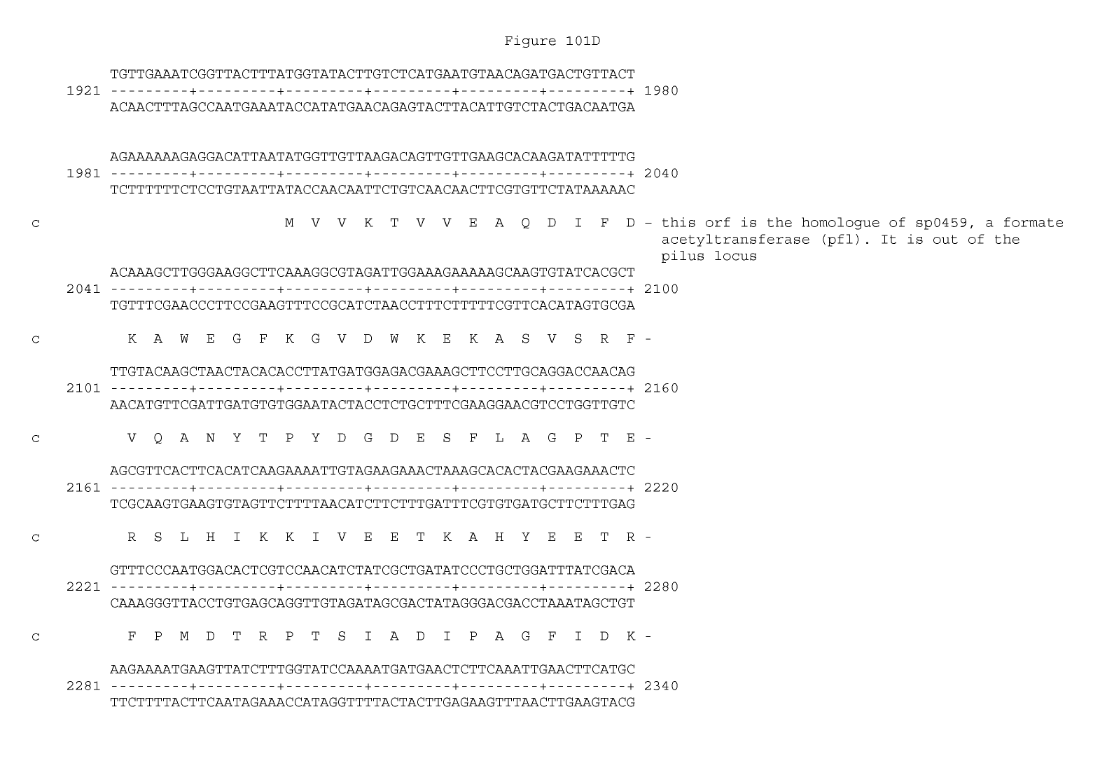

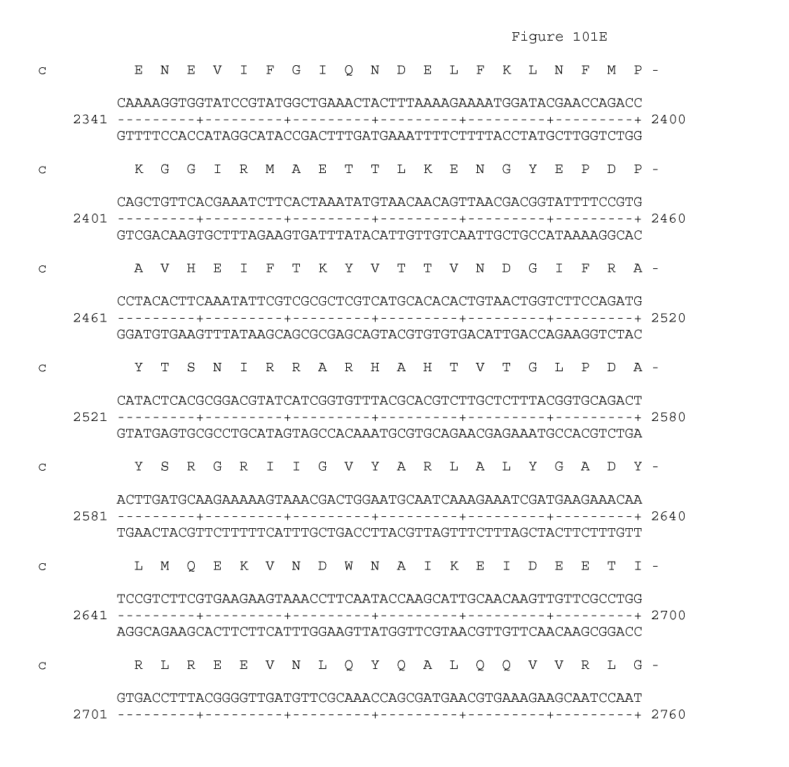

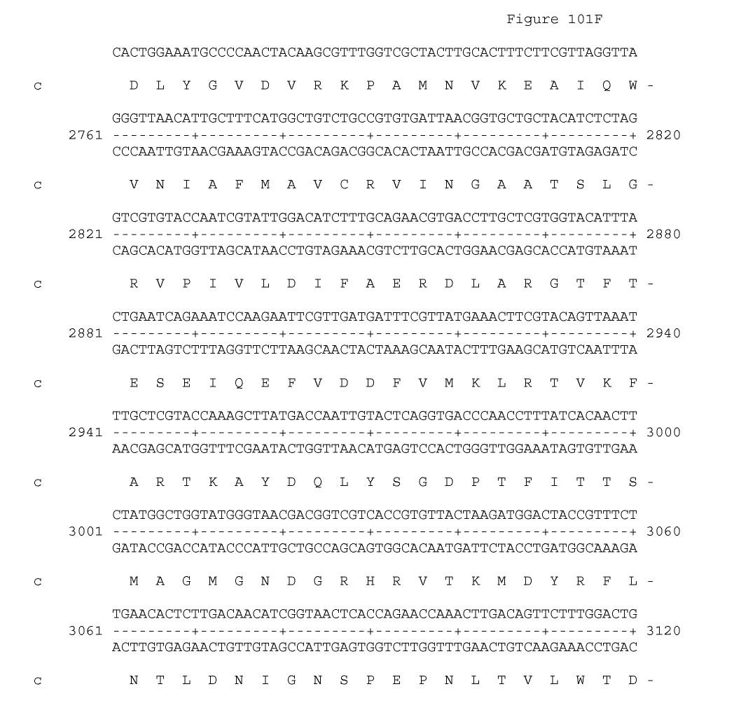

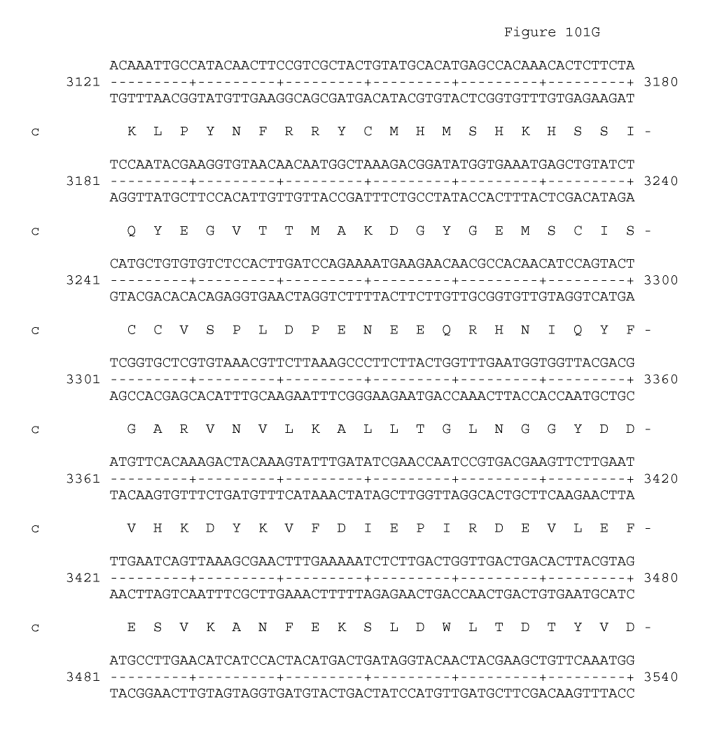

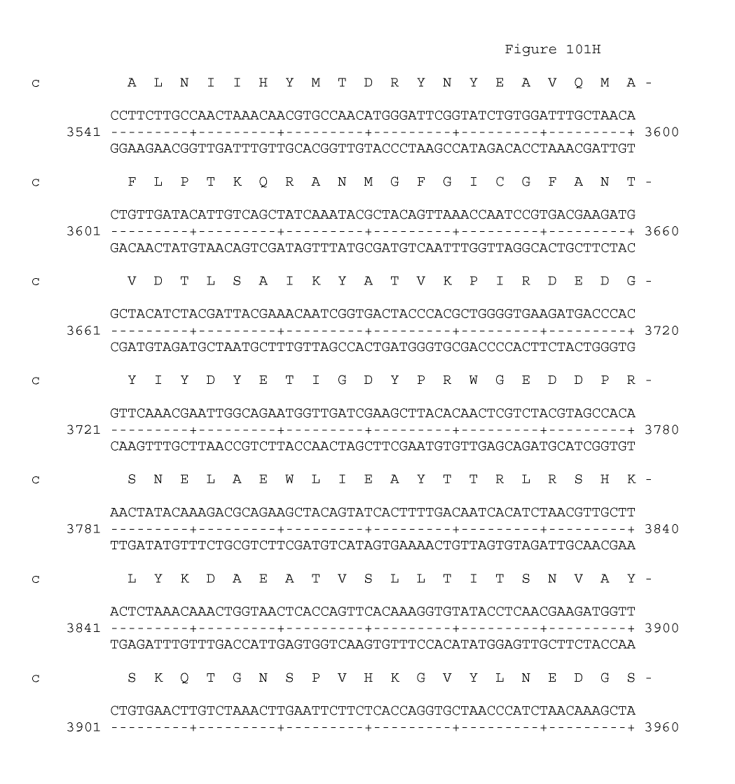

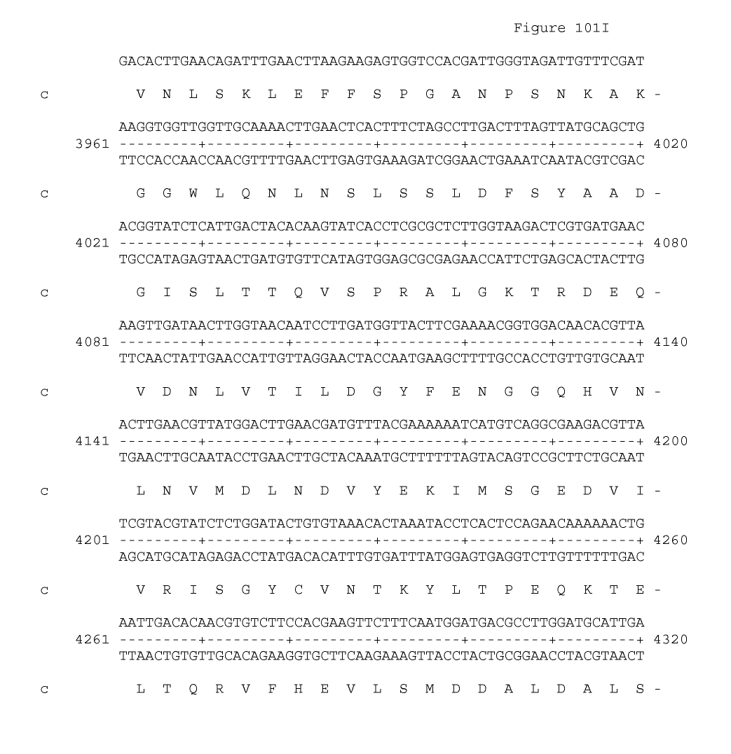

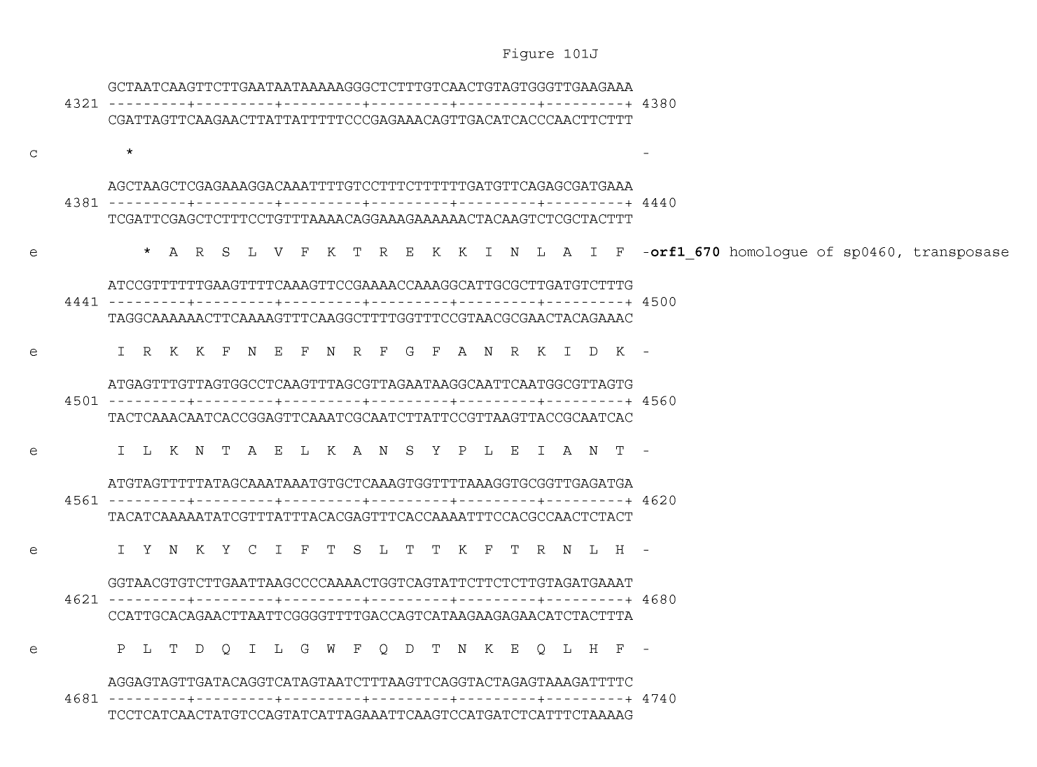

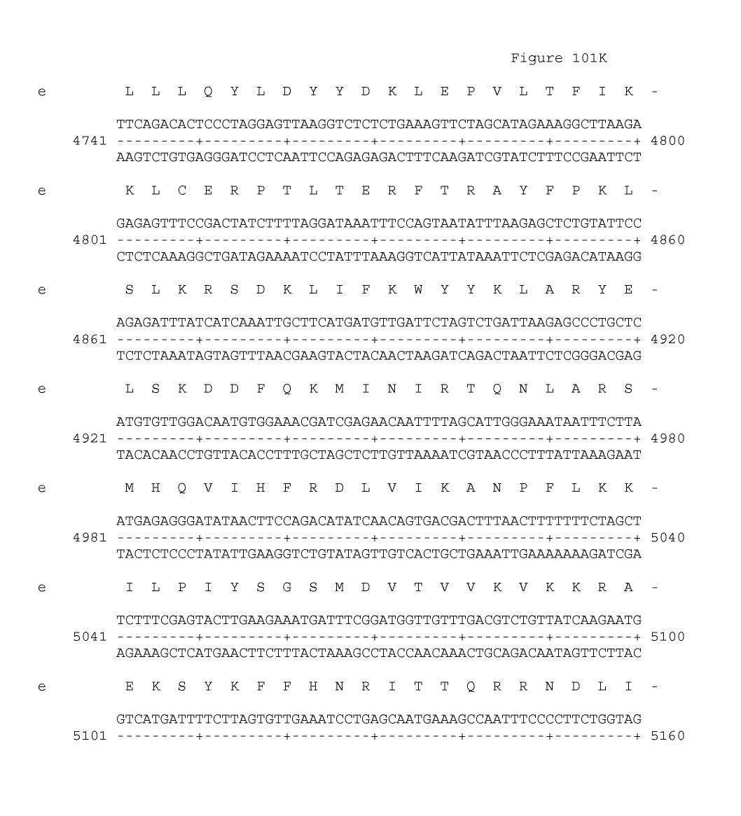

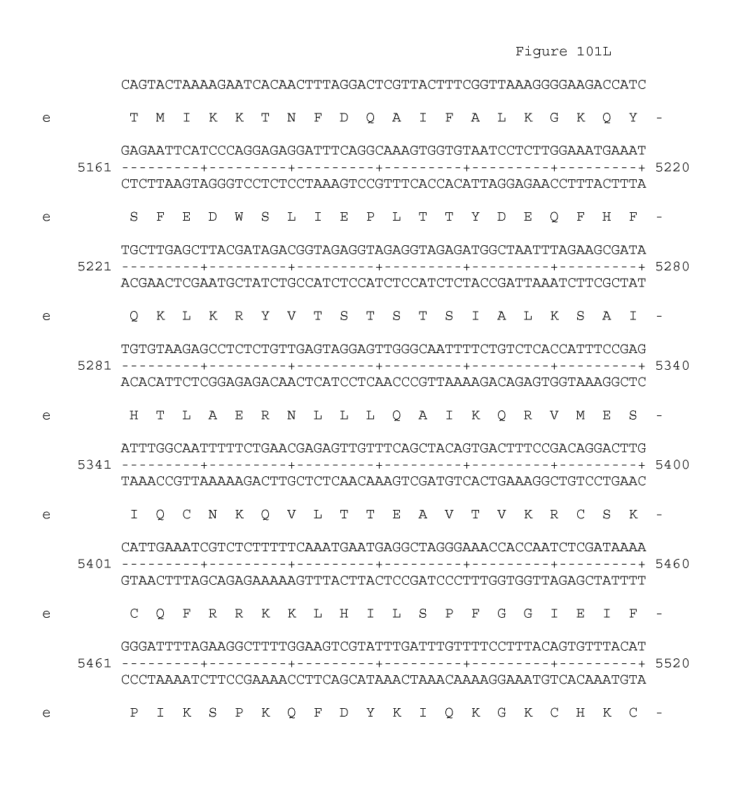

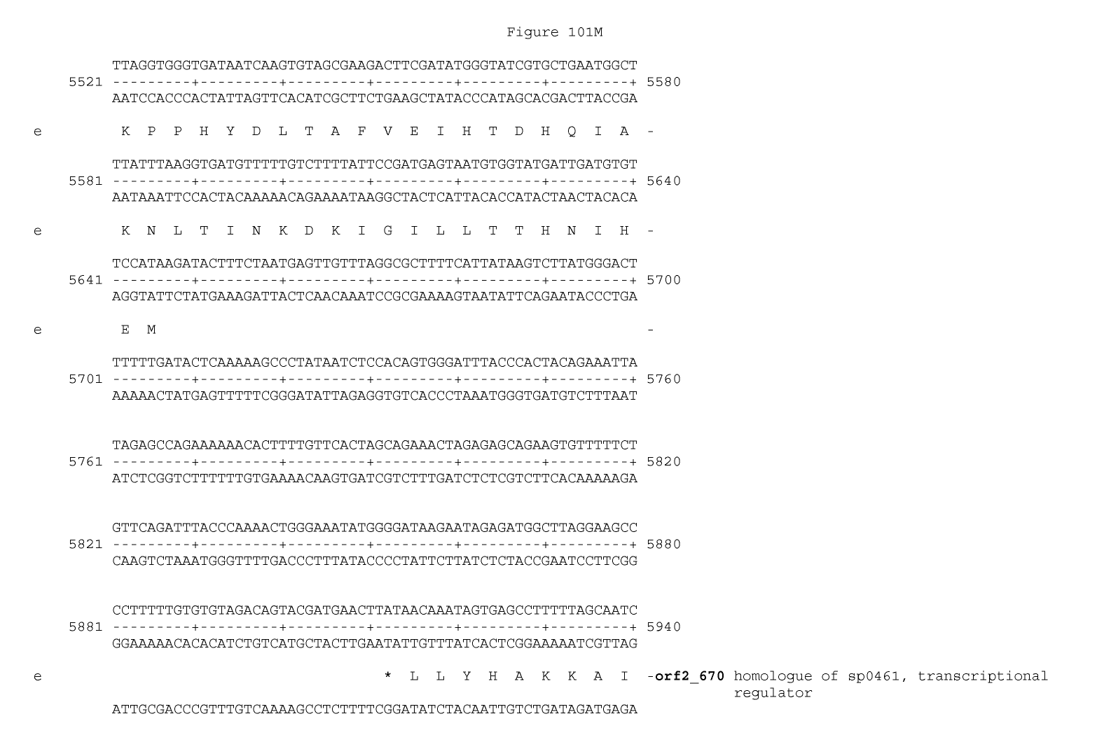

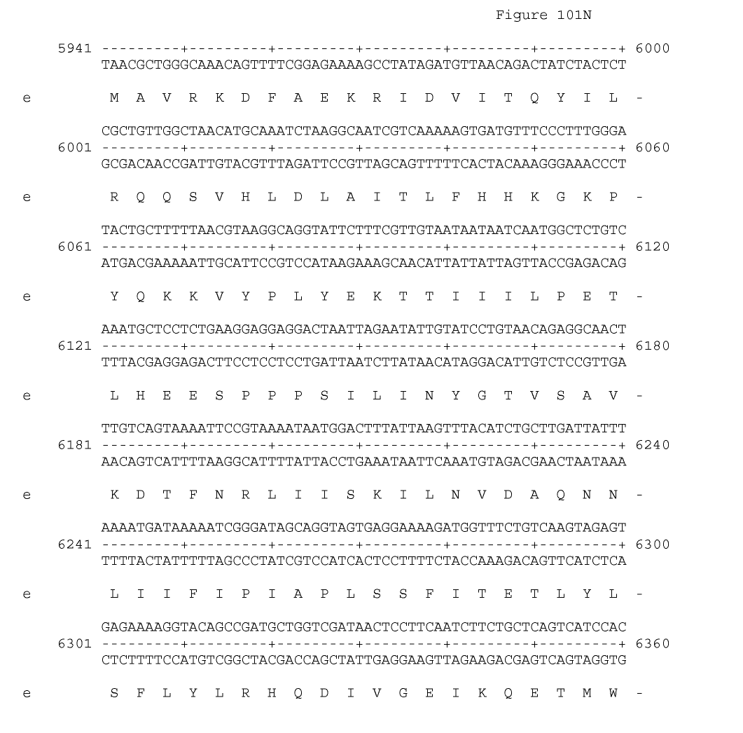

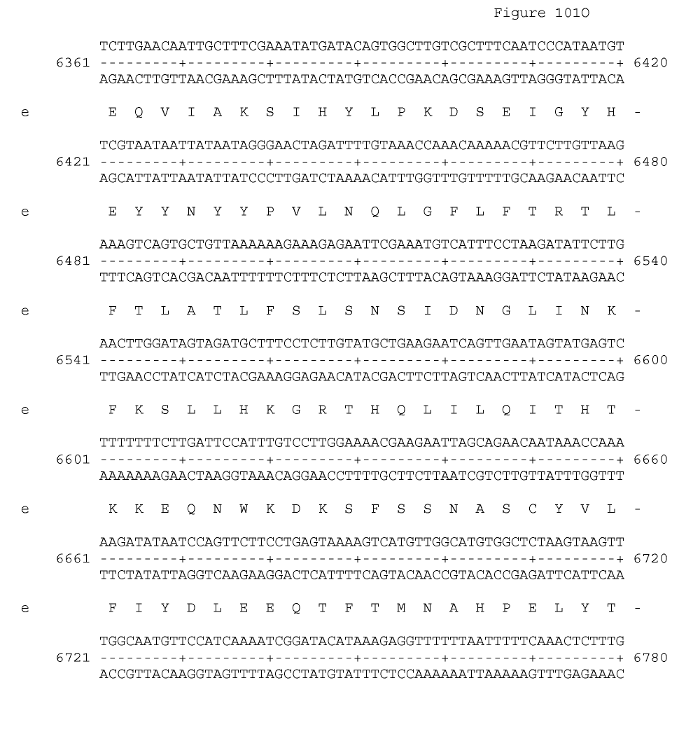

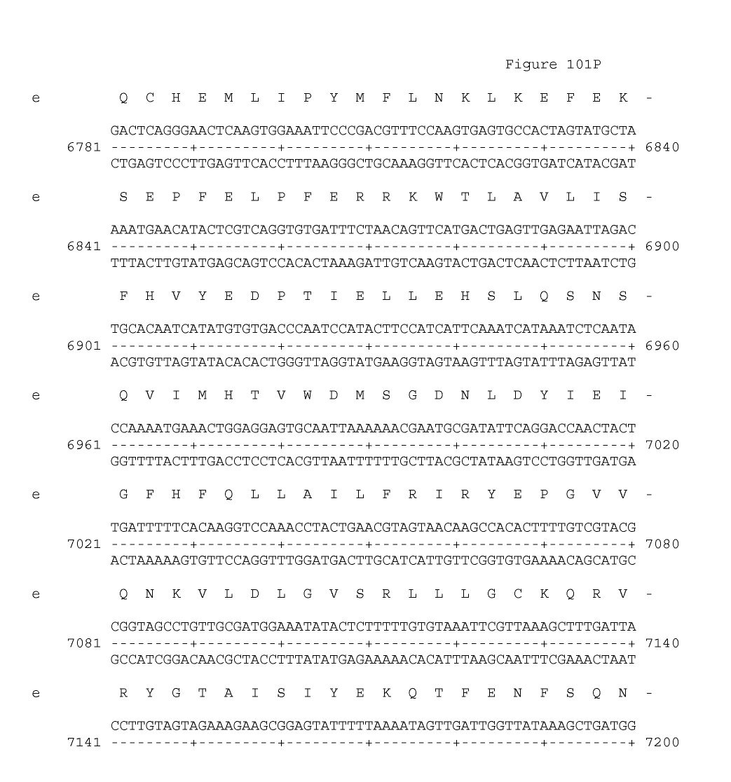

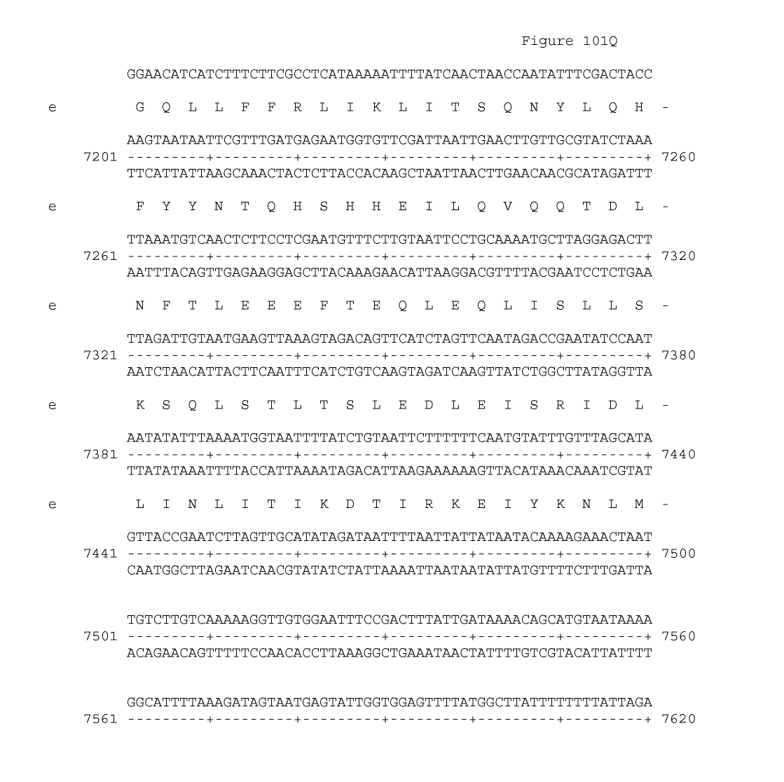

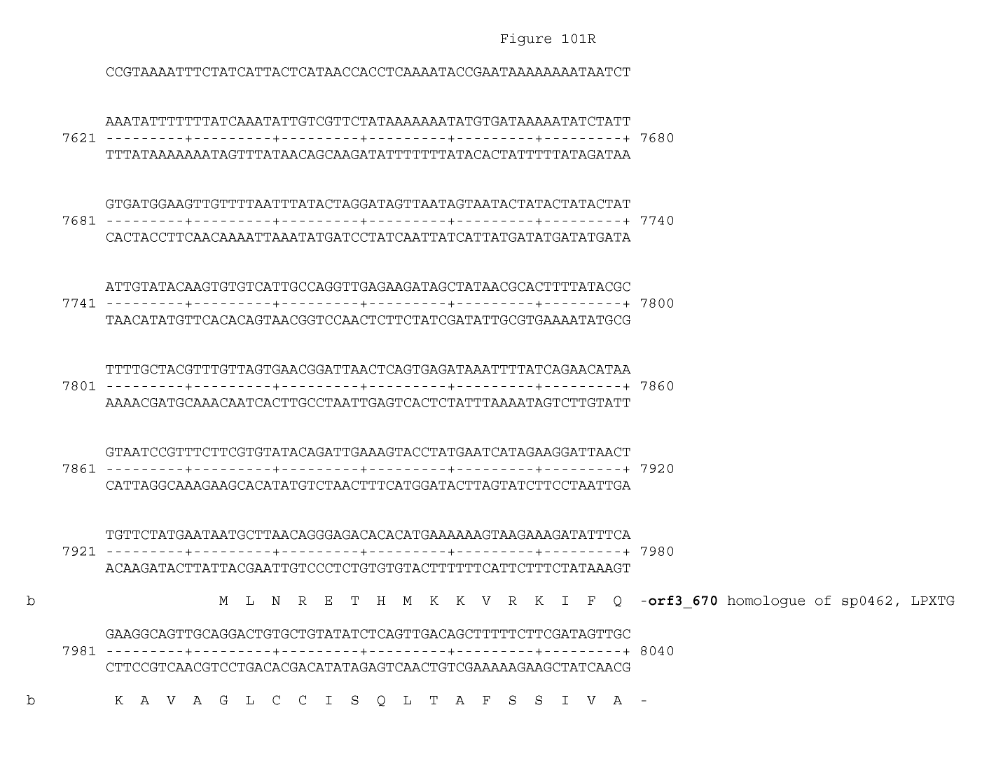

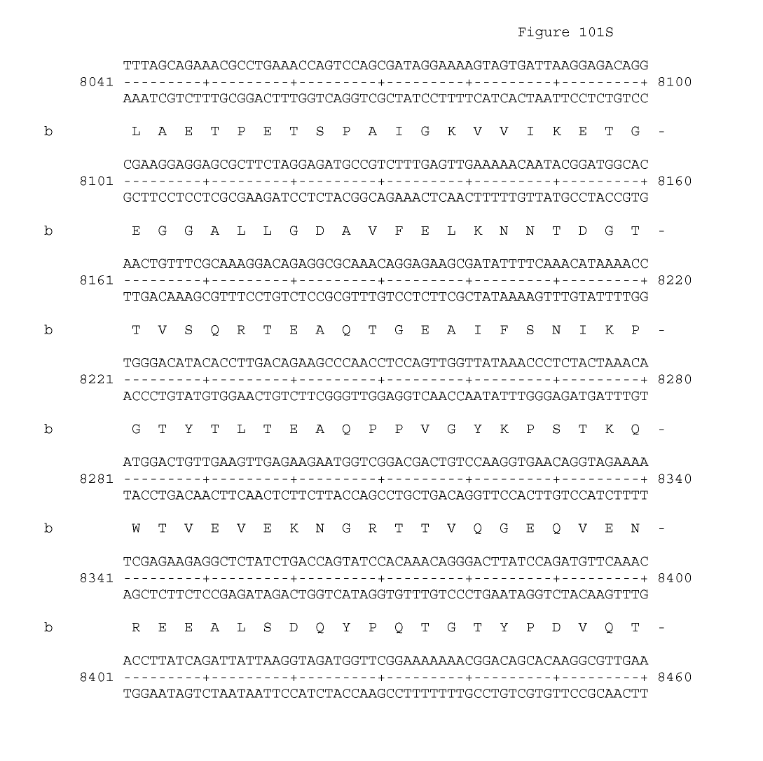

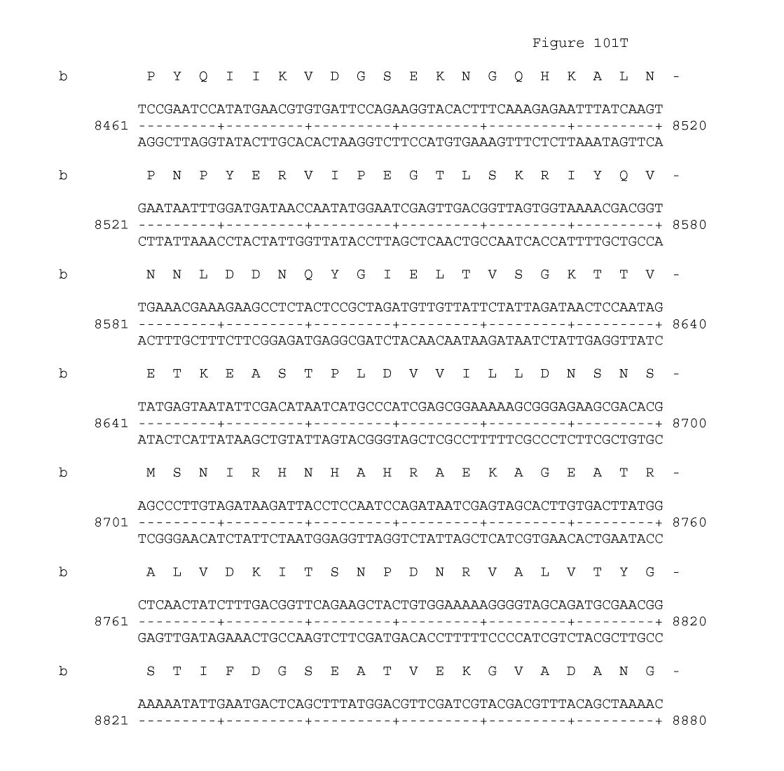

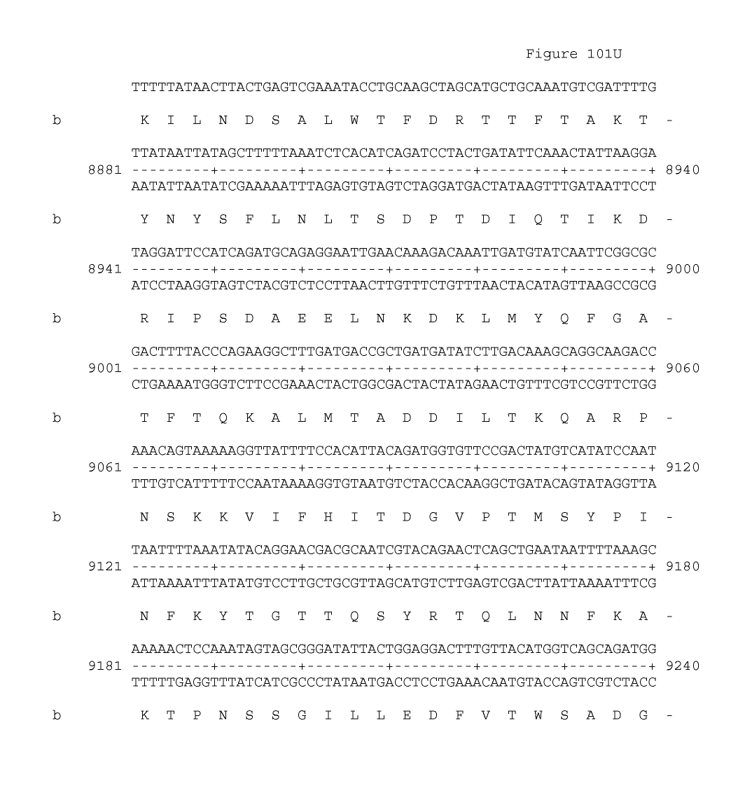

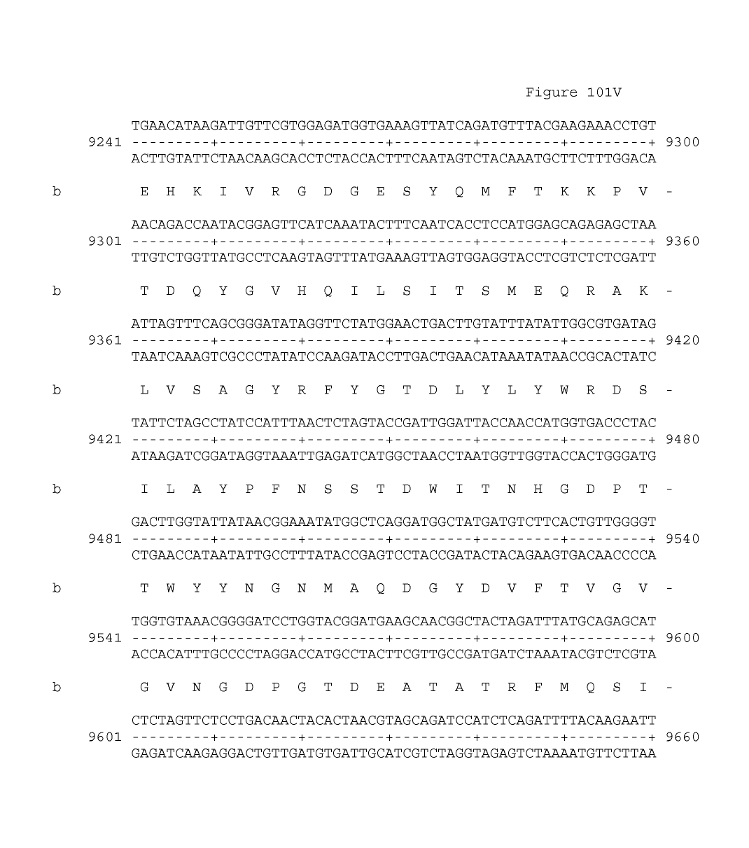

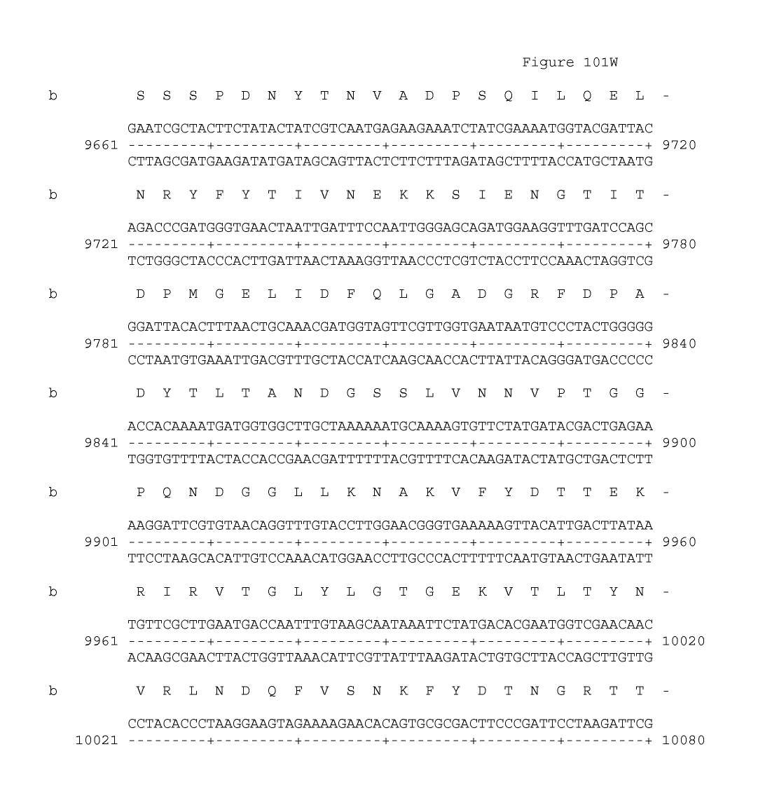

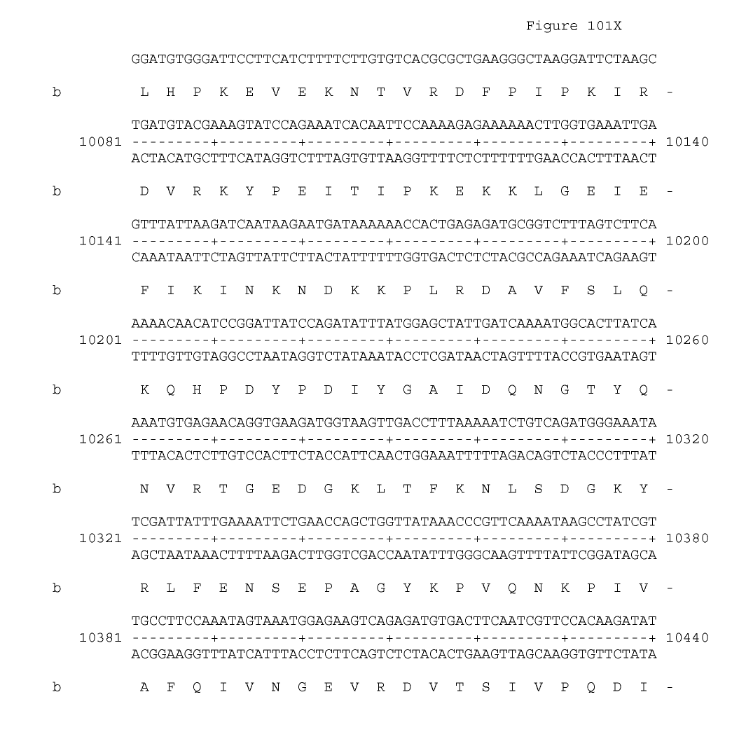

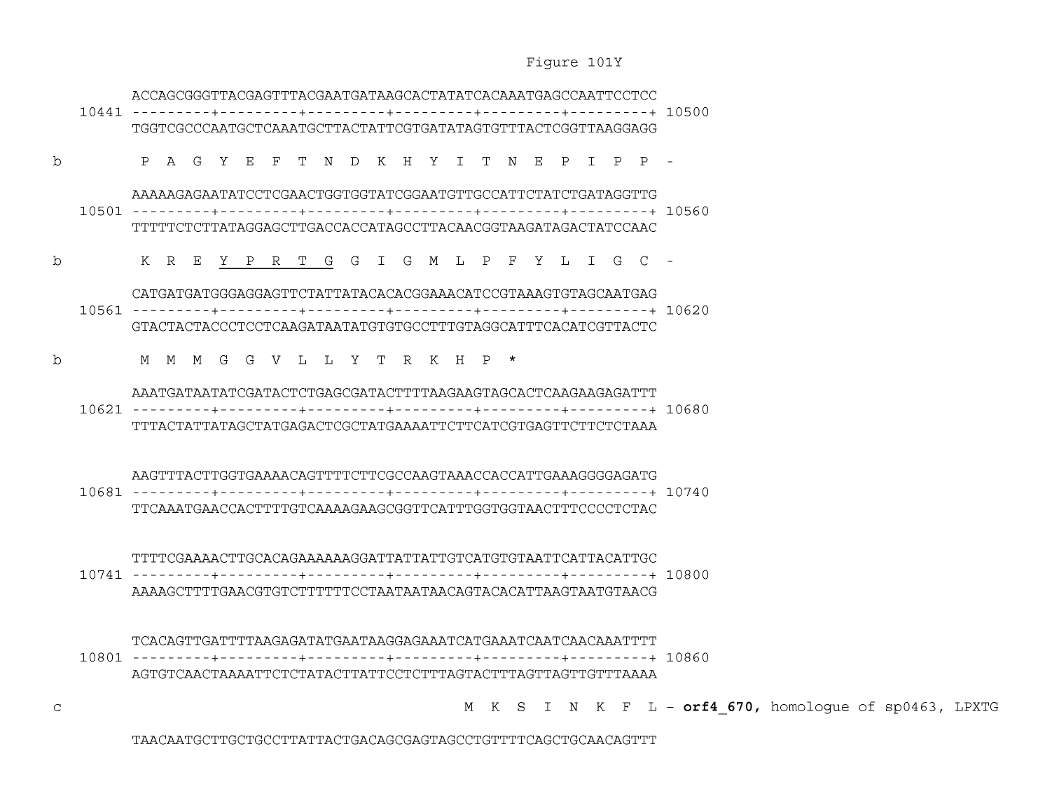

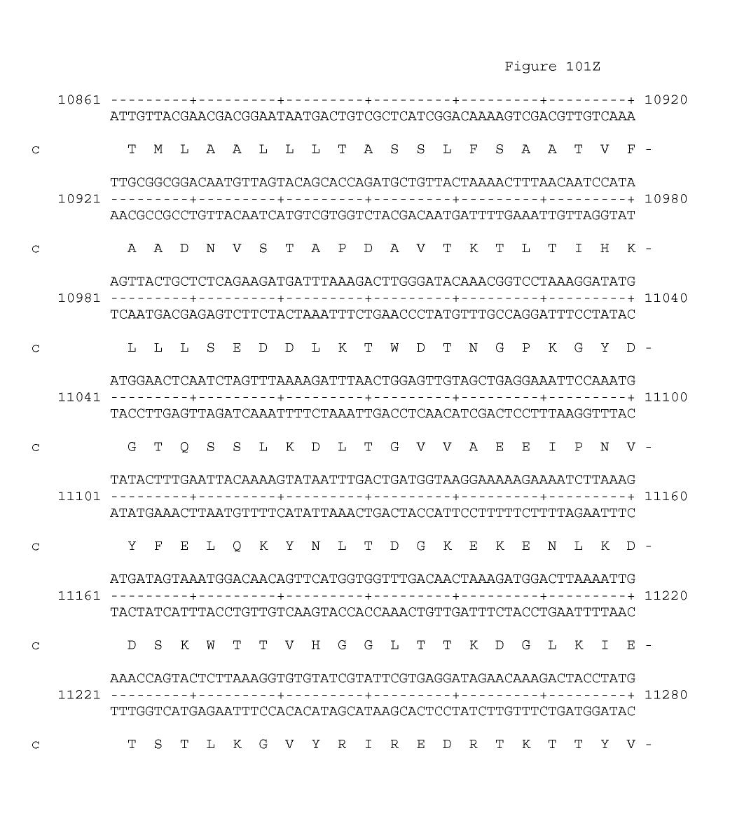

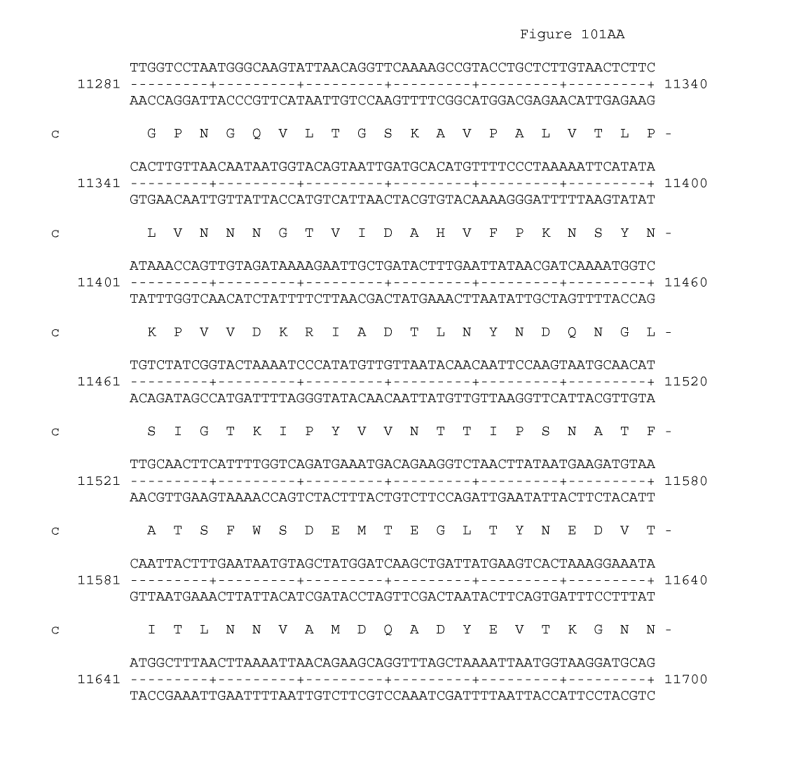

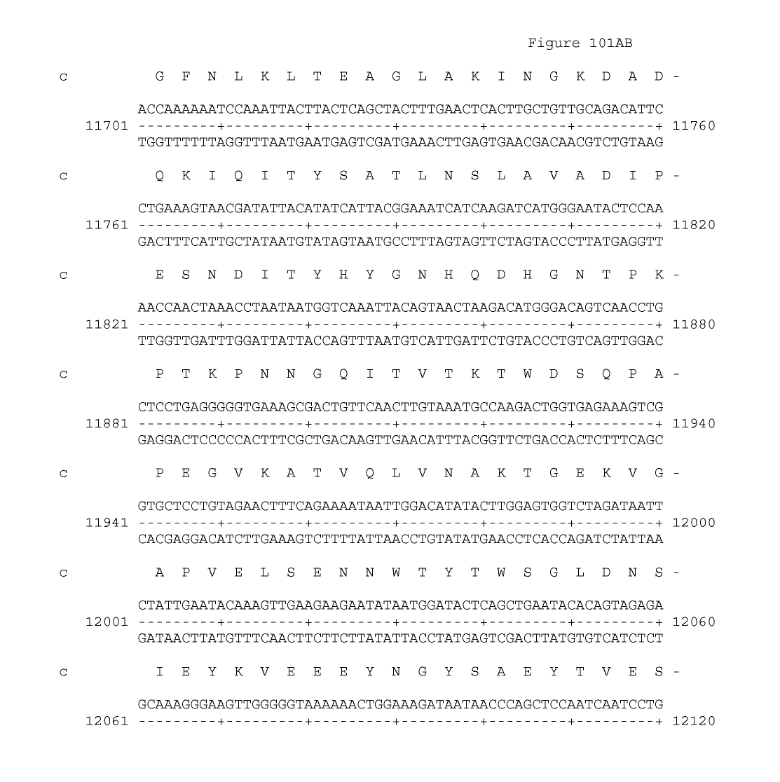

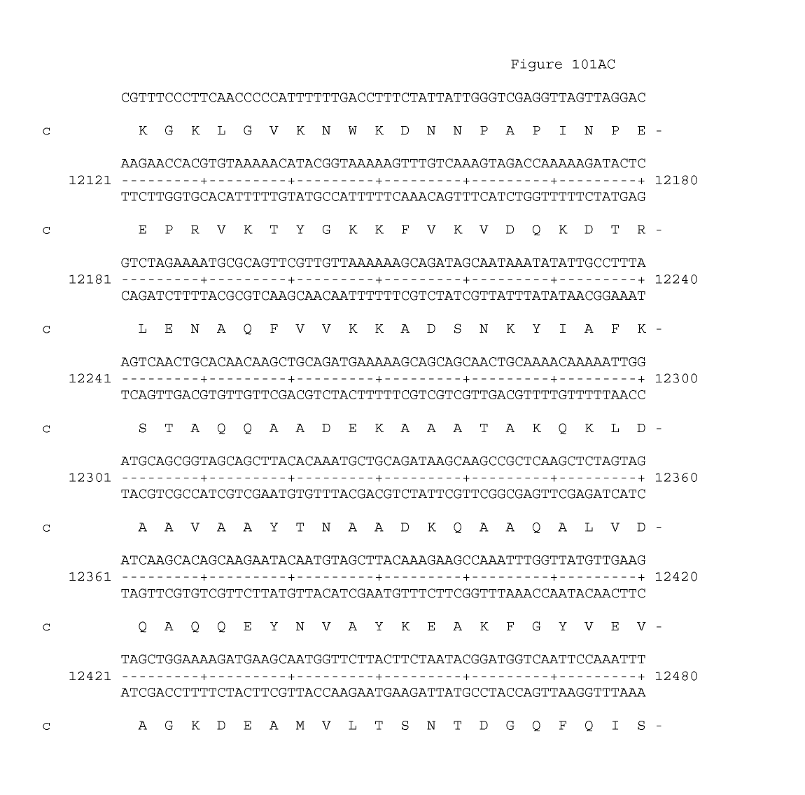

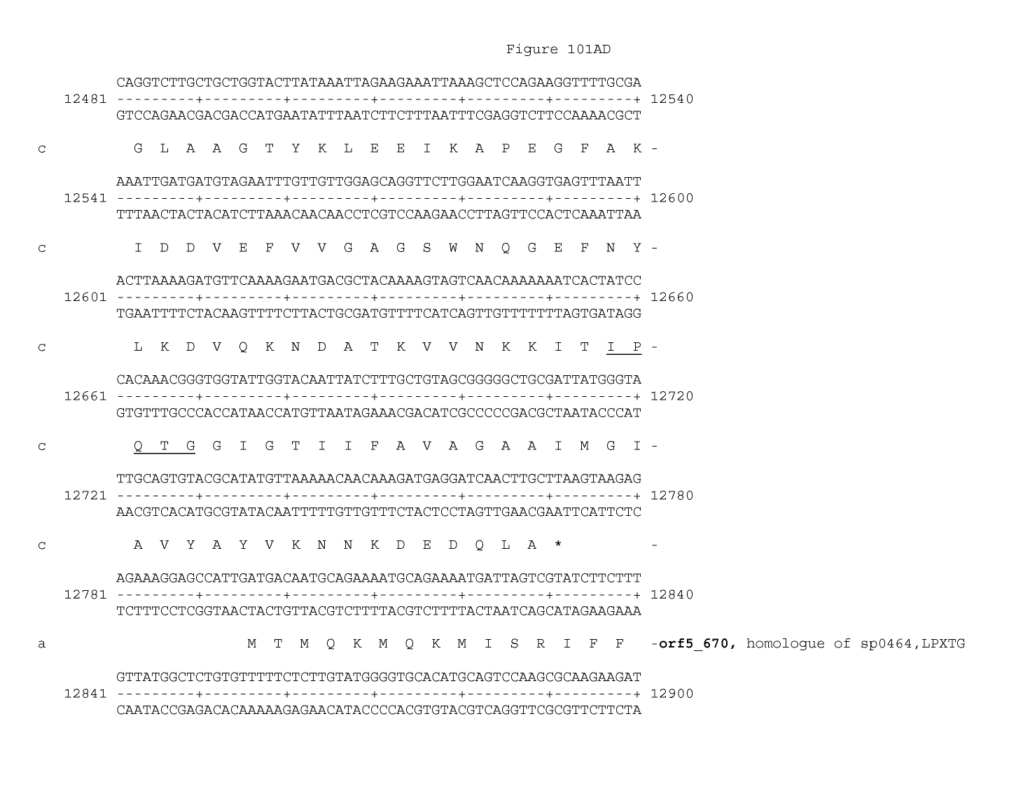

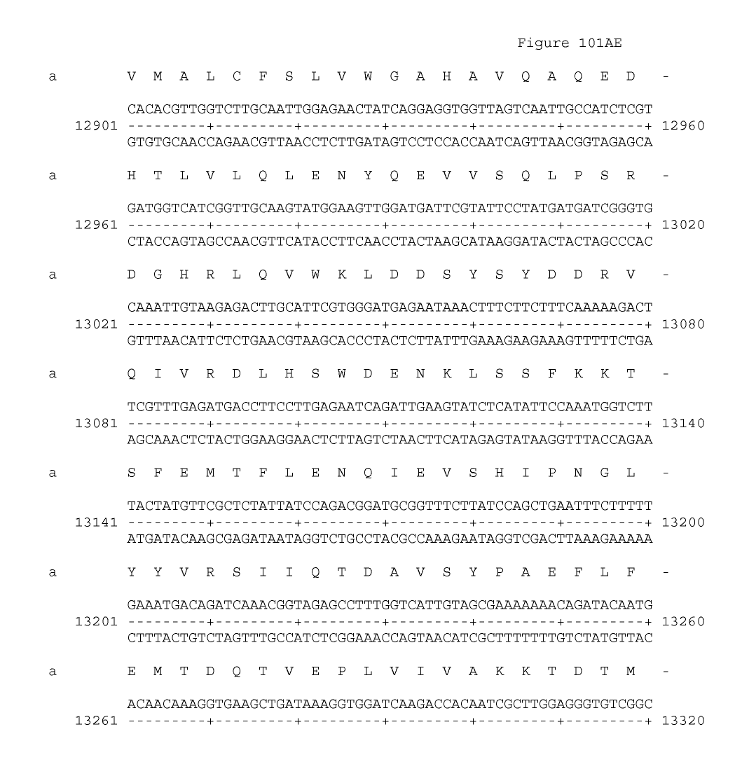

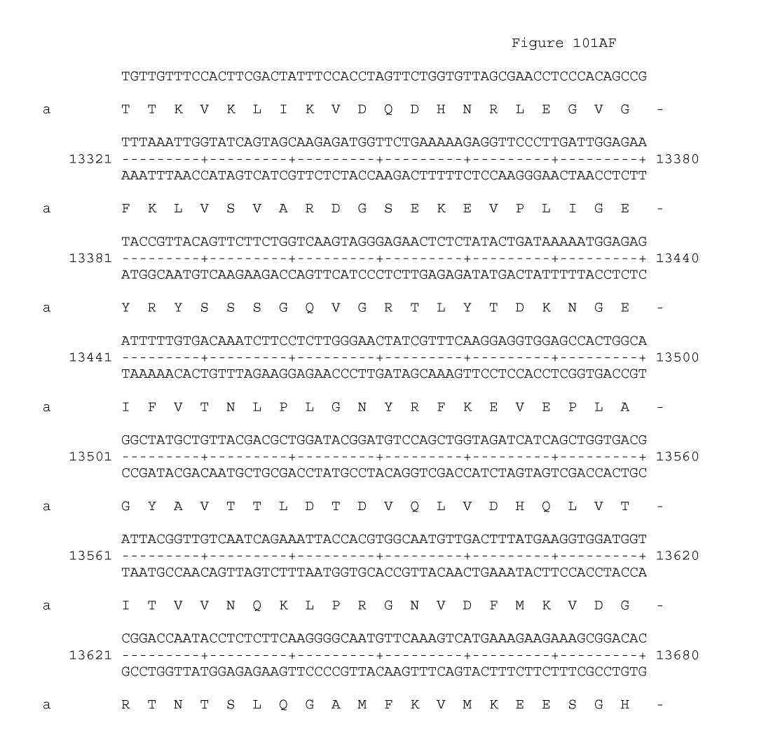

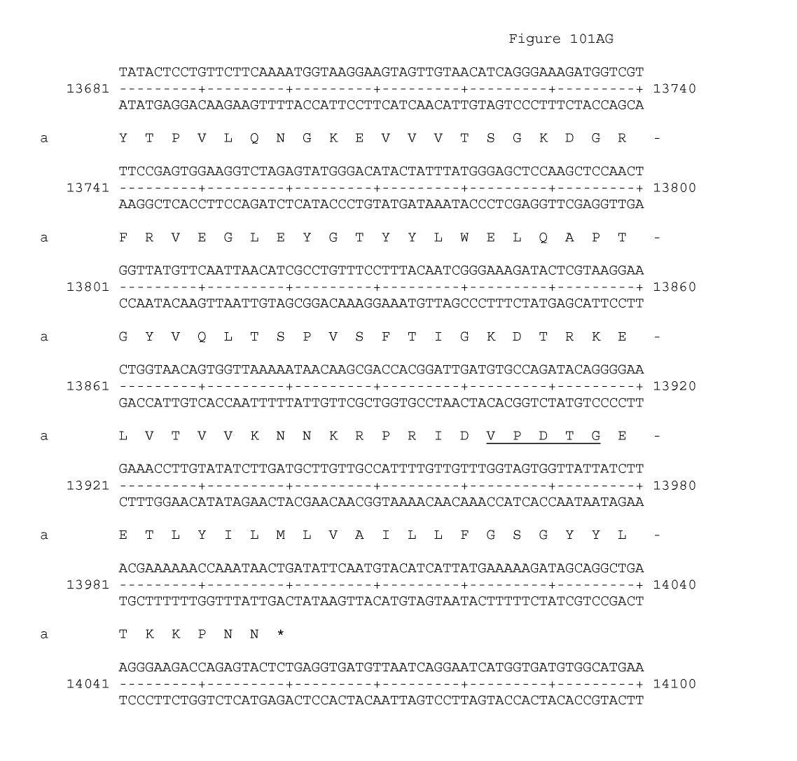

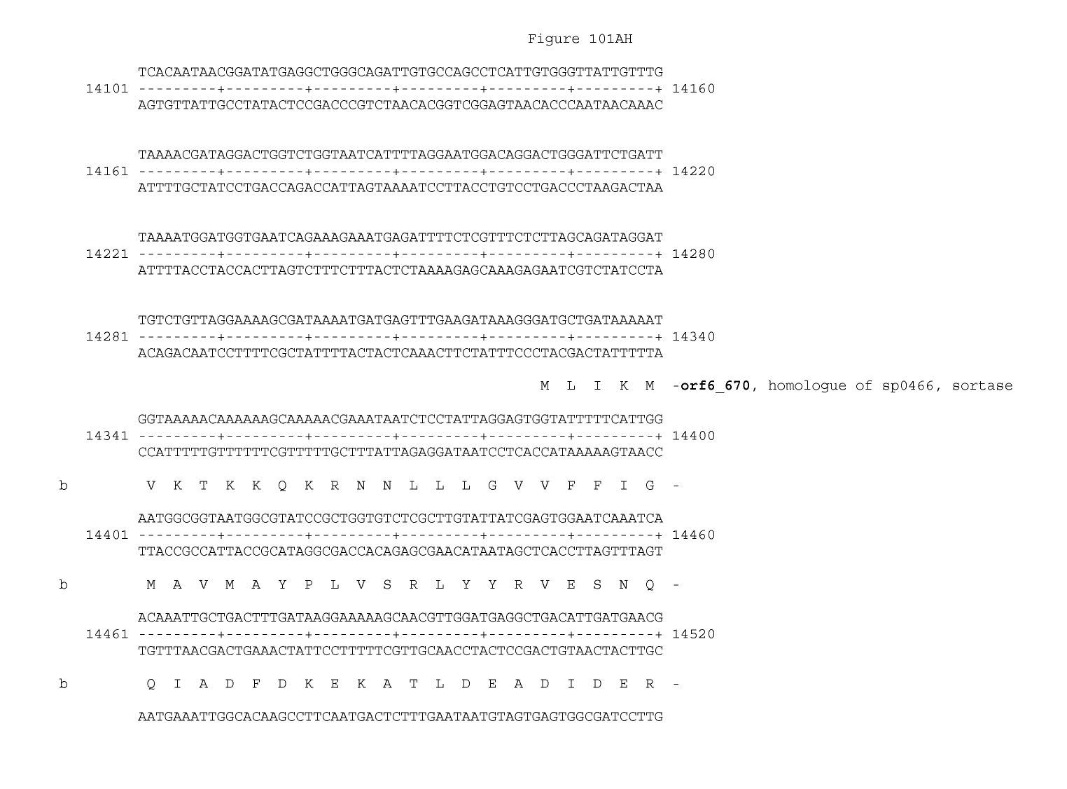

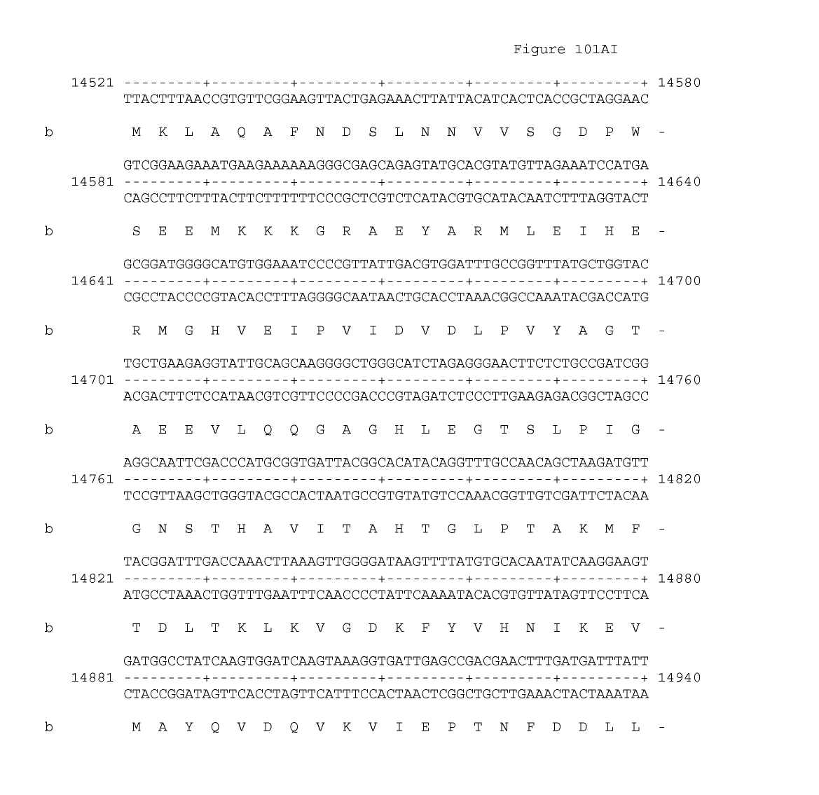

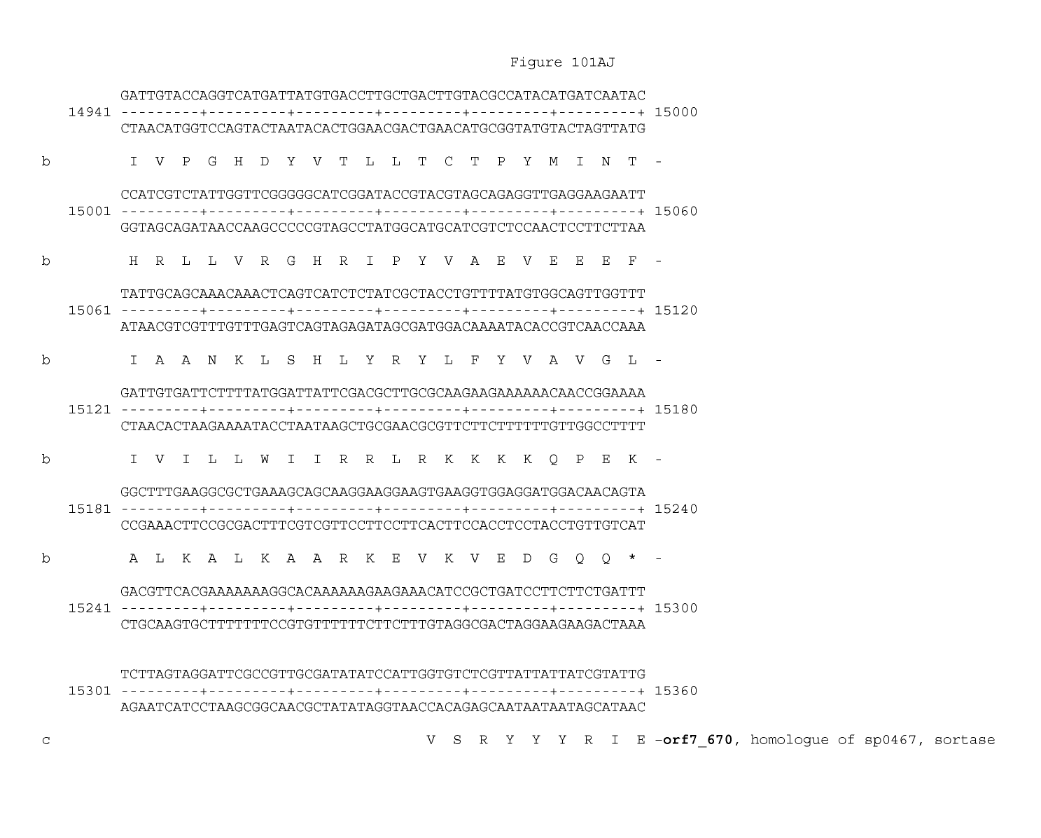

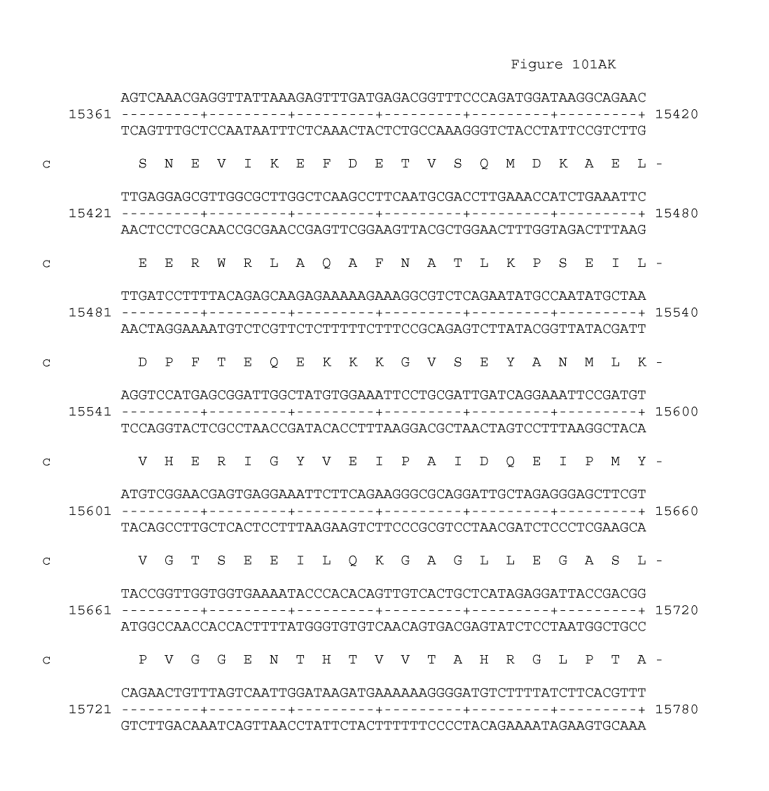

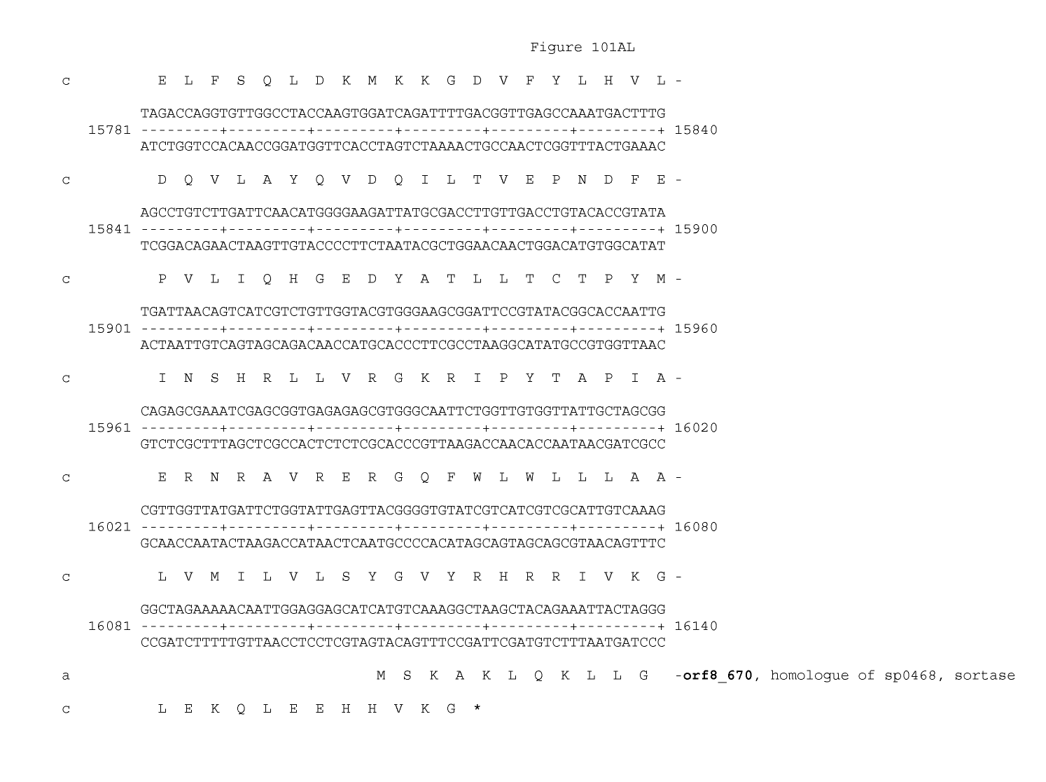

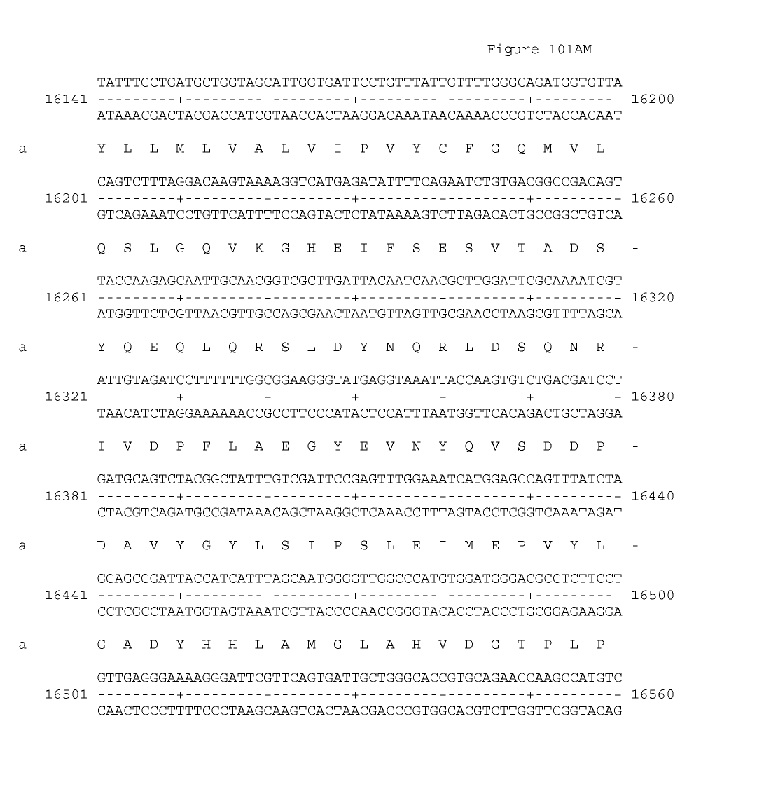

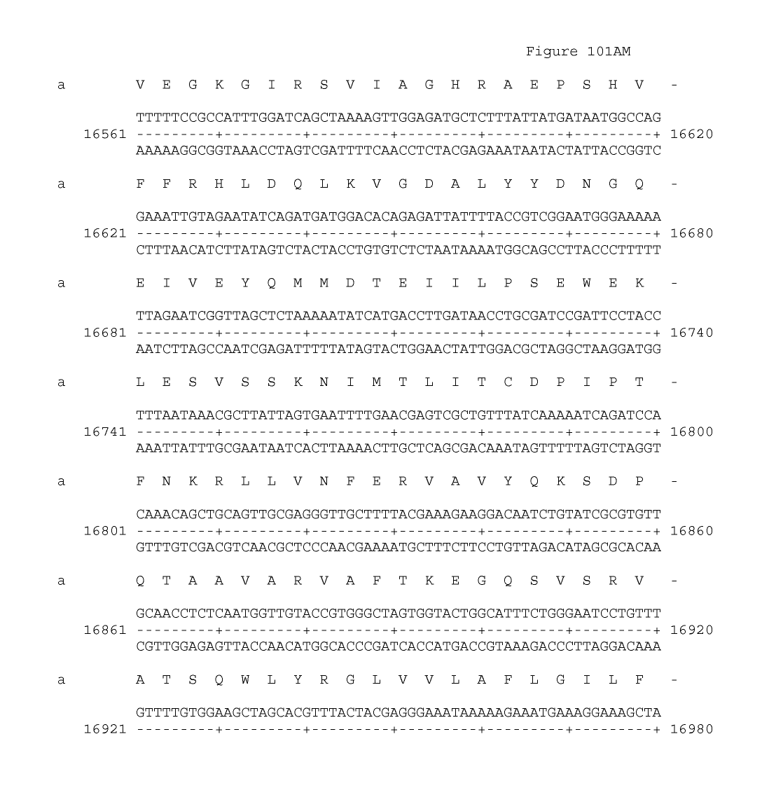

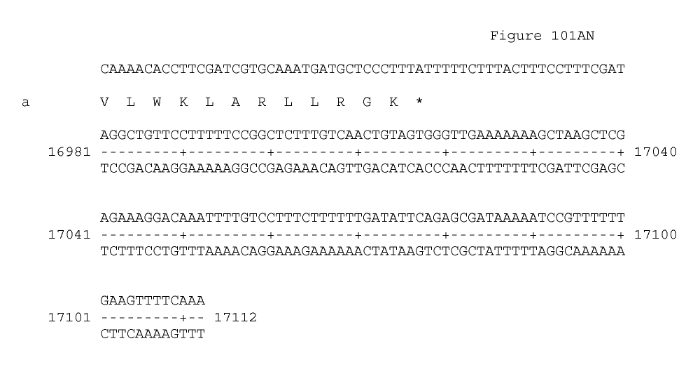

The full-length nucleotide sequence of the S. pneumoniae strain 670 AI is also shown in FIG. 101, as is its translated amino acid sequence.

At least eight other S. pneumoniae strains contain an adhesin island locus described by the locus depicted in FIG. 137. These strains were identified by an amplification analysis. The genomes of different S. pneumoniae strains were amplified with eleven separate sets of primers. The sequence of each of these primers is provided below in Table 41.

TABLE 41
Sequences of primers used to amplify AT locus

| Primer | Forward Primer | Reverse Primer |
|---|---|---|
| Pair | Sequence | Sequence |
| 1 | ACTTTCTAATGAGTTGTTTAG | AGCGACAAGCCACTGTATCAT |
| | GCG | ATT |
| | (SEQ ID NO: 274) | (SEQ ID NO: 285) |
| 2 | CTGGTCGATAACTCCTTCAAT | GTACGACAAAAGTGTGGCTTG |
| | CTT | TT |
| | (SEQ ID NO: 275) | (SEQ ID NO: 286) |
| 3 | GAATGCGATATTCAGGACCAA | ATCTCACTGAGTTAATCCGTT |
| | CTA | CAC |
| | (SEQ ID NO: 276) | (SEQ ID NO: 287) |
| 4 | TGTATACAAGTGTGTCATTGC | CATCTTCACCTGTTCTCACAT |
| | CAG | TTT |
| | (SEQ ID NO: 277) | (SEQ ID NO: 288) |
| 5 | GCGGTCTTTAGTCTTCAAAAA | CAAGAGAAAAACACAGAGCCA |
| | CA | TAA |
| | (SEQ ID NO: 278) | (SEQ ID NO: 289) |
| 6 | TTGCTTAAGTAAGAGAGAAAG | CAGGAGTATAGTGTCCGCTTT |
| | GAGC | CTT |
| | (SEQ ID NO: 279) | (SEQ ID NO: 290) |
| 7 | GGCAATGTTGACTTTATGAAG | TATCAGCATCCCTTTATCTTC |
| | GTG | AAAC |
| | (SEQ ID NO: 280) | (SEQ ID NO: 291) |
| 8 | TGAGATTTTCTCGTTTCTCTT | AATAGACGATGGGTATTGATC |
| | AGC | ATGT |
| | (SEQ ID NO: 281) | (SEQ ID NO: 292) |
| 9 | CCGACGAACTTTGATGATTTA | ACCAACAGACGATGACTGTTA |
| | TTG | ATC |
| | (SEQ ID NO: 282) | (SEQ ID NO: 293) |
| 10 | AATGACTTTGAGCCTGTCTTG | TTCTACAATTTCCTGGCCATT |
| | AT | ATC |
| | (SEQ ID NO: 283) | (SEQ ID NO: 294) |
| 11 | GCCATTTGGATCAGCTAAAAG | TTTTTCAACCCACTACAGTTG |
| | TT | ACA |
| | (SEQ ID NO: 284) | (SEQ ID NO: 295) |

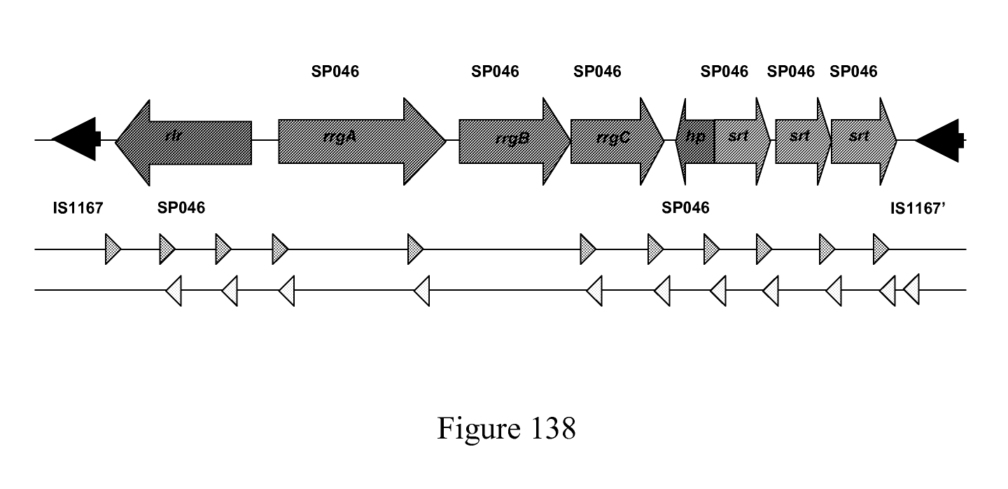

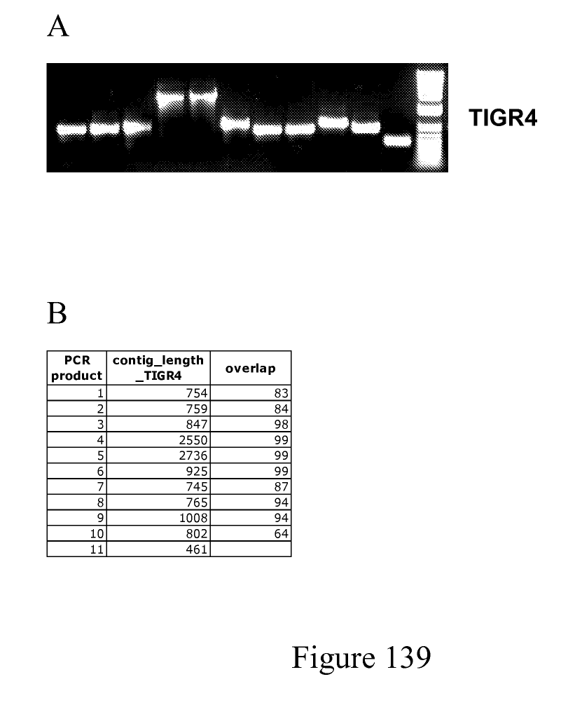

These primers hybridized along the entire length of the AI locus to generate amplification products representative of sequences throughout the locus. See FIG. 138, which is a schematic of the location where each of these primers hybridizes to the S. pneumoniae AI locus. FIG. 139A provides the set of amplicons obtained from amplification of the AI locus in S. pneumoniae strain TIGR4. FIG. 139B provides the length, in base pairs, of each amplicon in S. pneumoniae strain TIGR4. Amplification of the genome of S. pneumoniae strains 19A Hungary 6, 6B Finland 12, 6B Spain 2, 9V Spain 3, 14 CSR 10, 19F Taiwan 14, 23F Taiwan 15, and 23F Poland 16 produced a set of eleven amplicons for the eleven primer pairs, indicating that each of these strains also contained the S. pneumoniae AI locus.

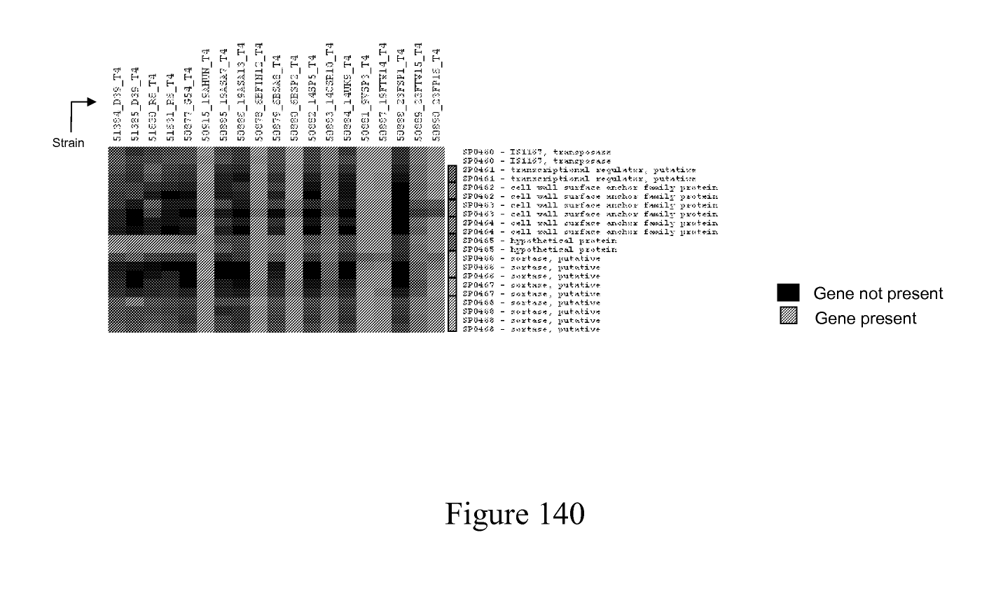

The S. pneumoniae strains were also identified as containing the AI locus by comparative genome hybridization (CGH) analysis. The genomes of sixteen S. pneumoniae strains were interrogated for the presence of the AI locus by comparison to unique open reading frames of strain TIGR4. The AI locus was detected by this method in strains 19A Hungary 6 (19AHUN), 6B Finland 12 (6BFIN12), 6B Spain 2 (6BSP2), 14CSR10 (14 CSR10), 9V Spain 3 (9VSP3), 19F Taiwan 14 (19FTW14), 23F Taiwan 15 (19FTW15), and 23F Poland 16 (23FP16). See FIG. 140.

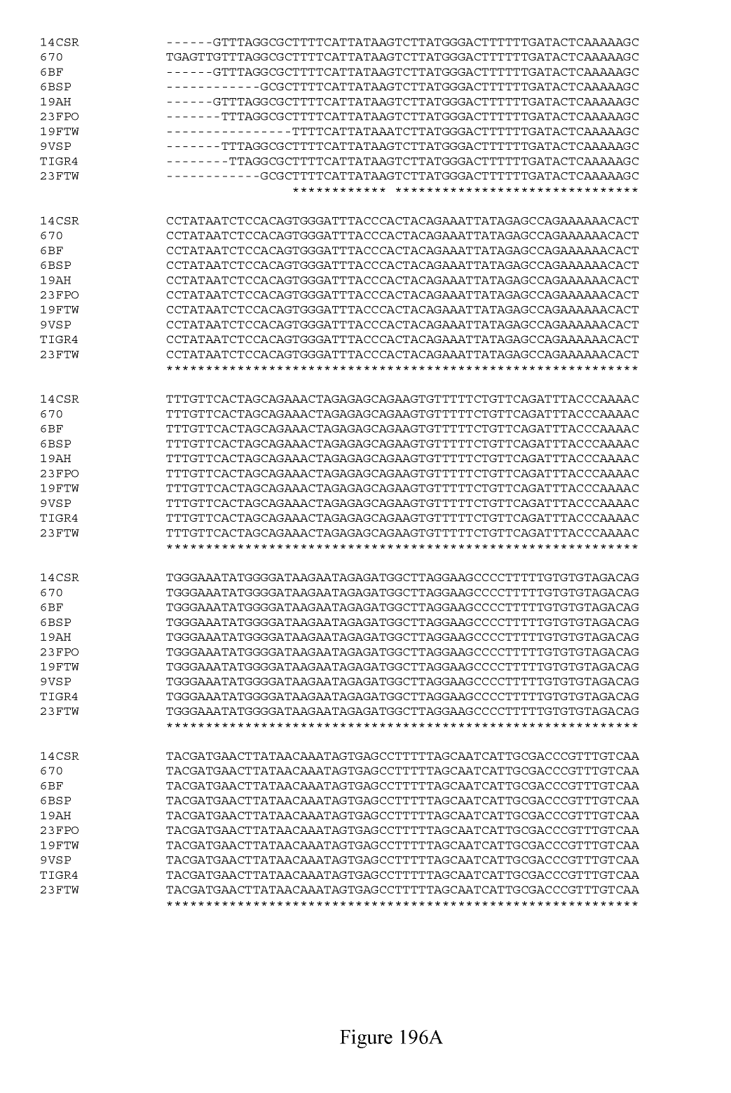

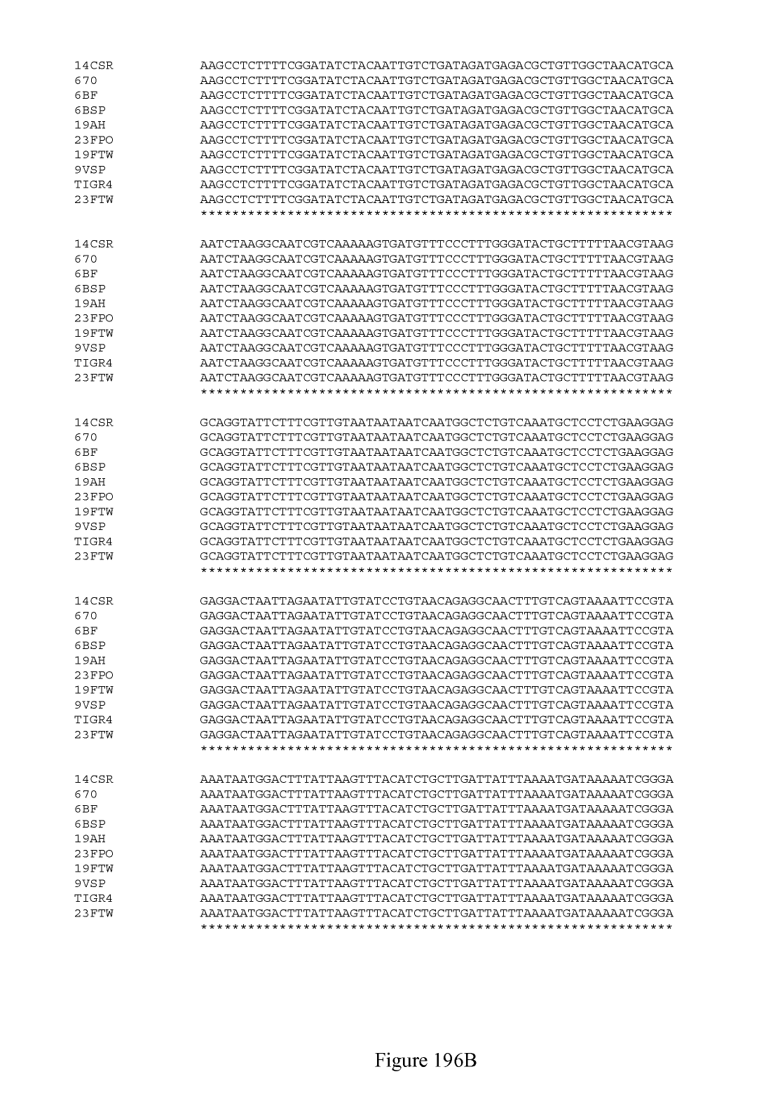

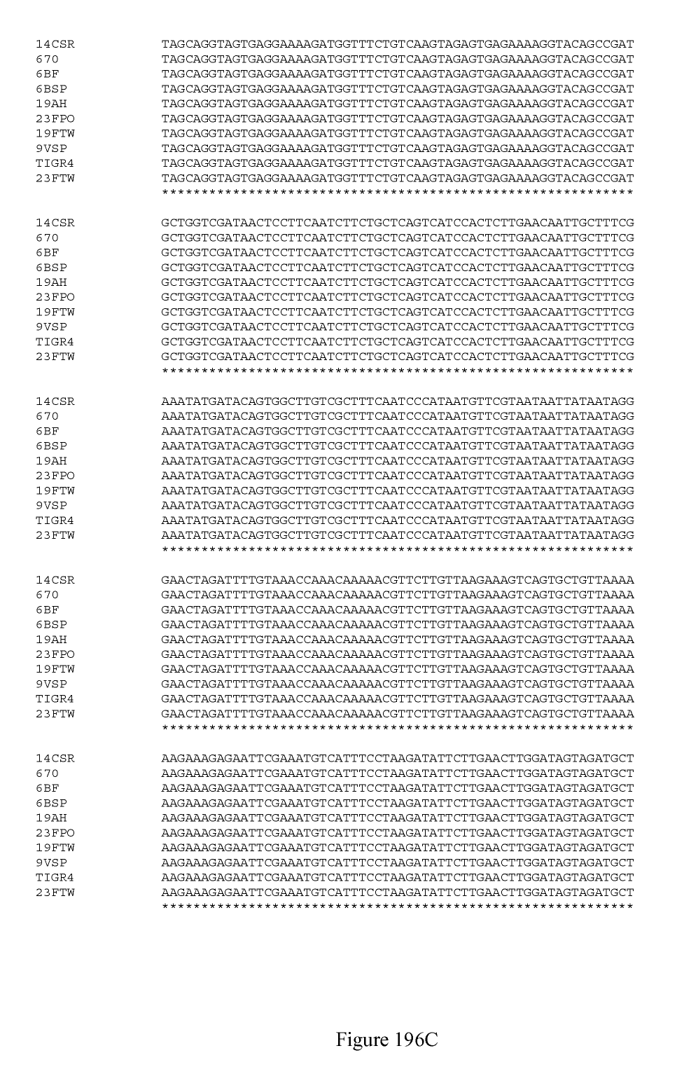

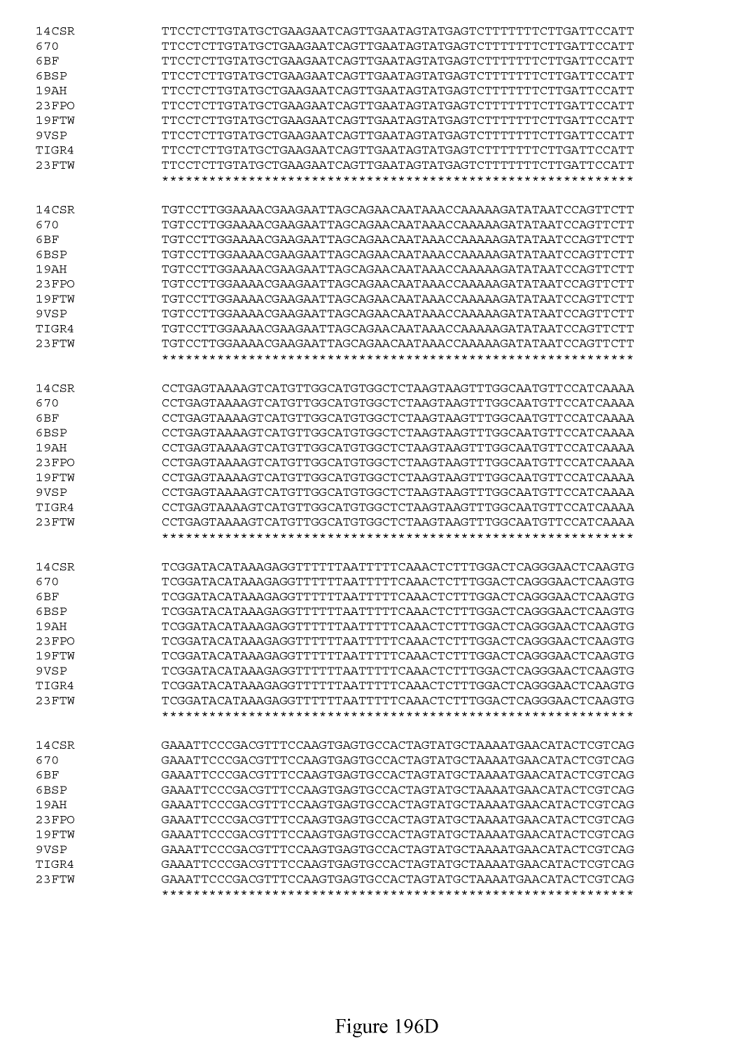

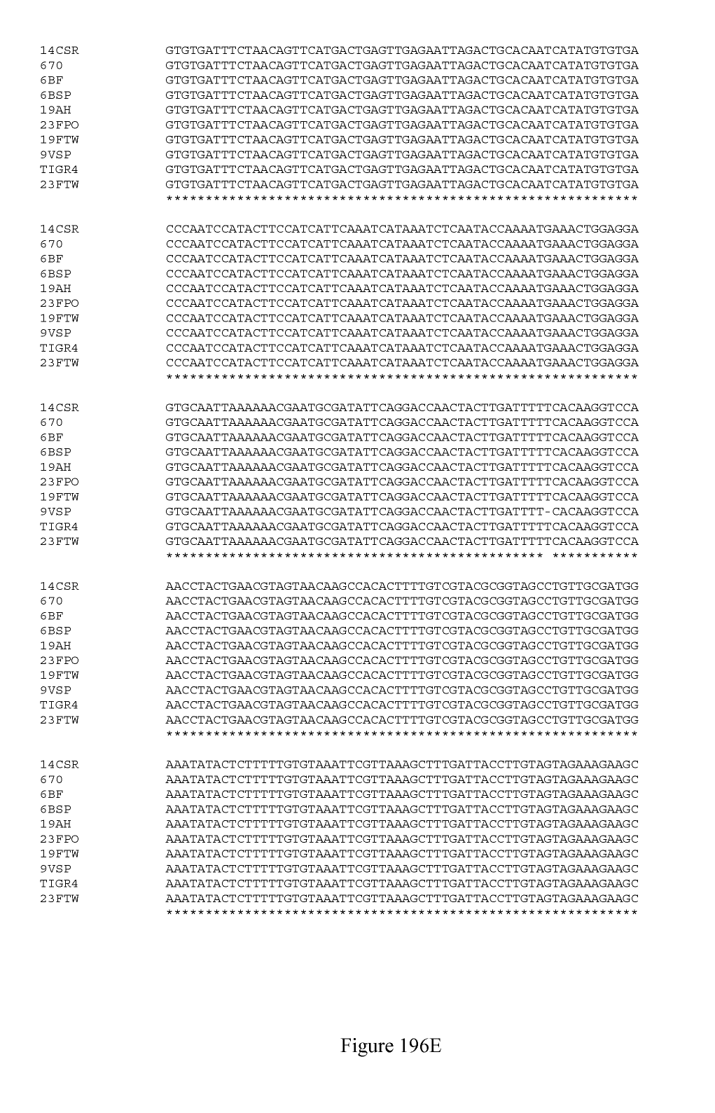

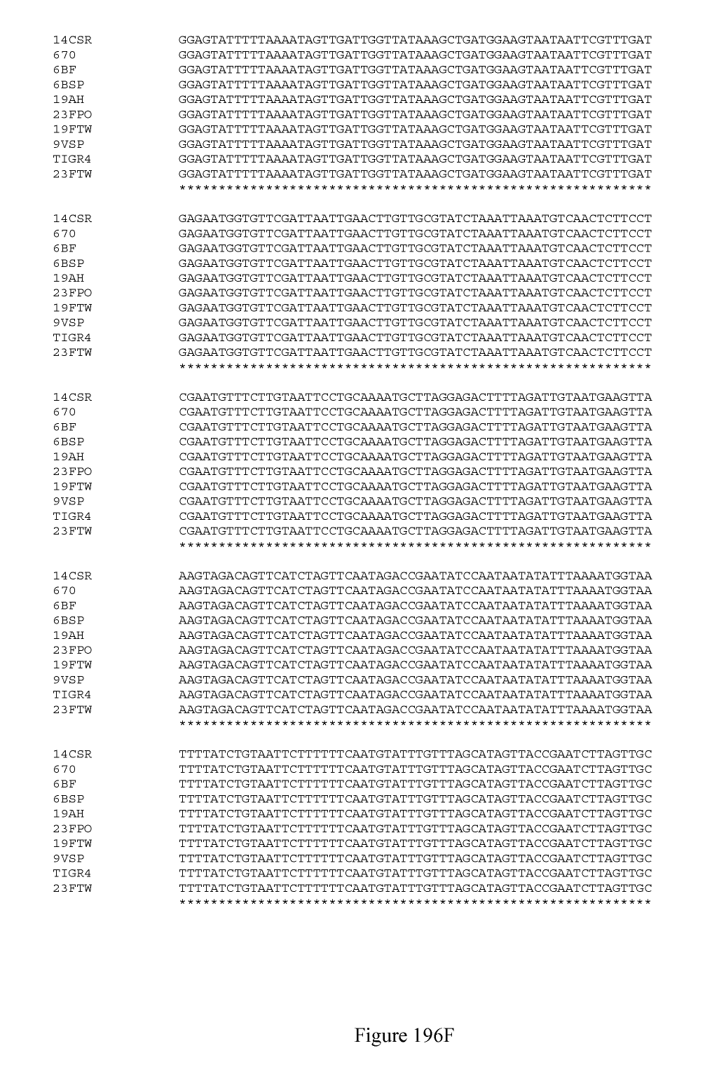

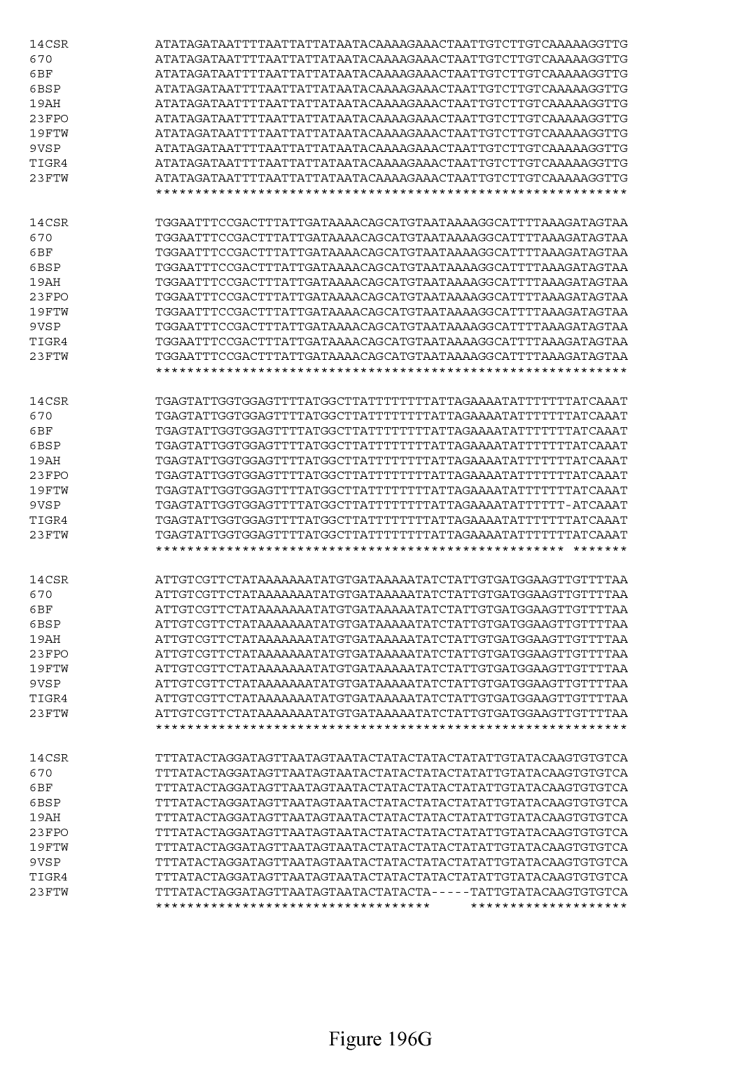

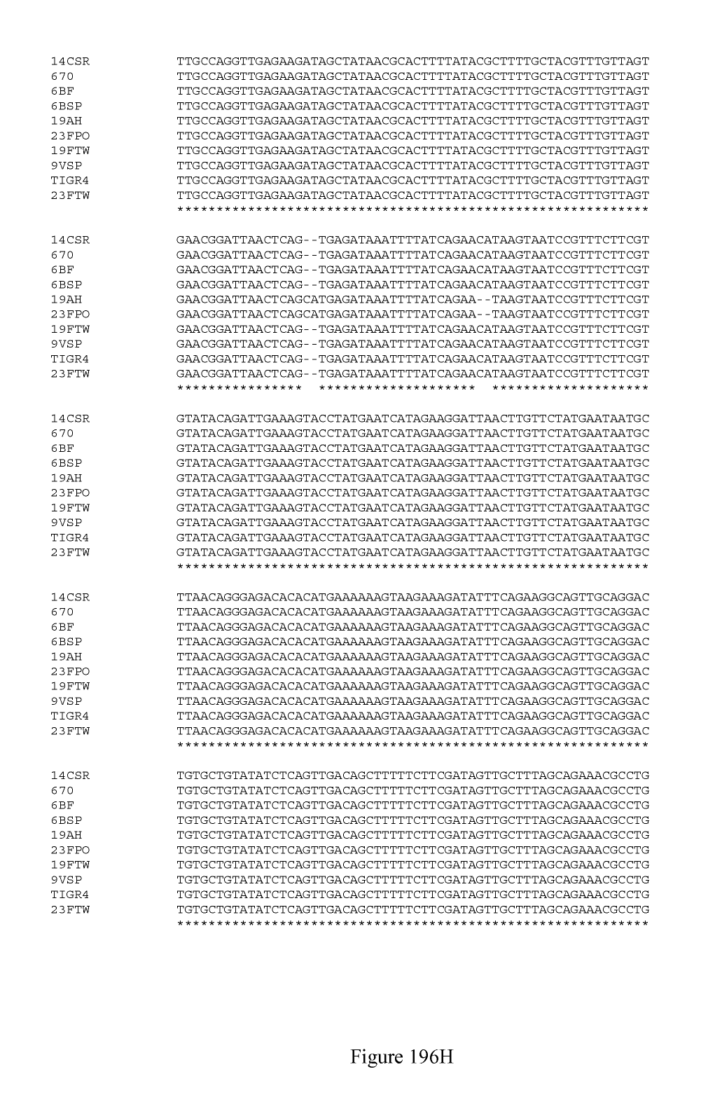

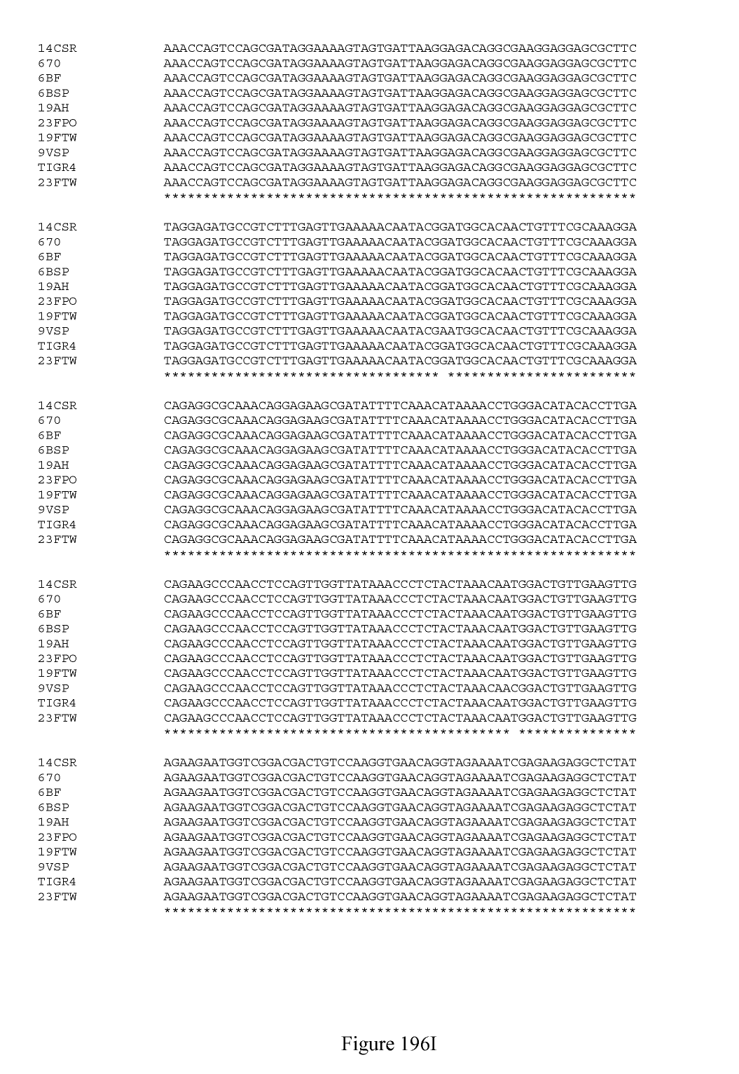

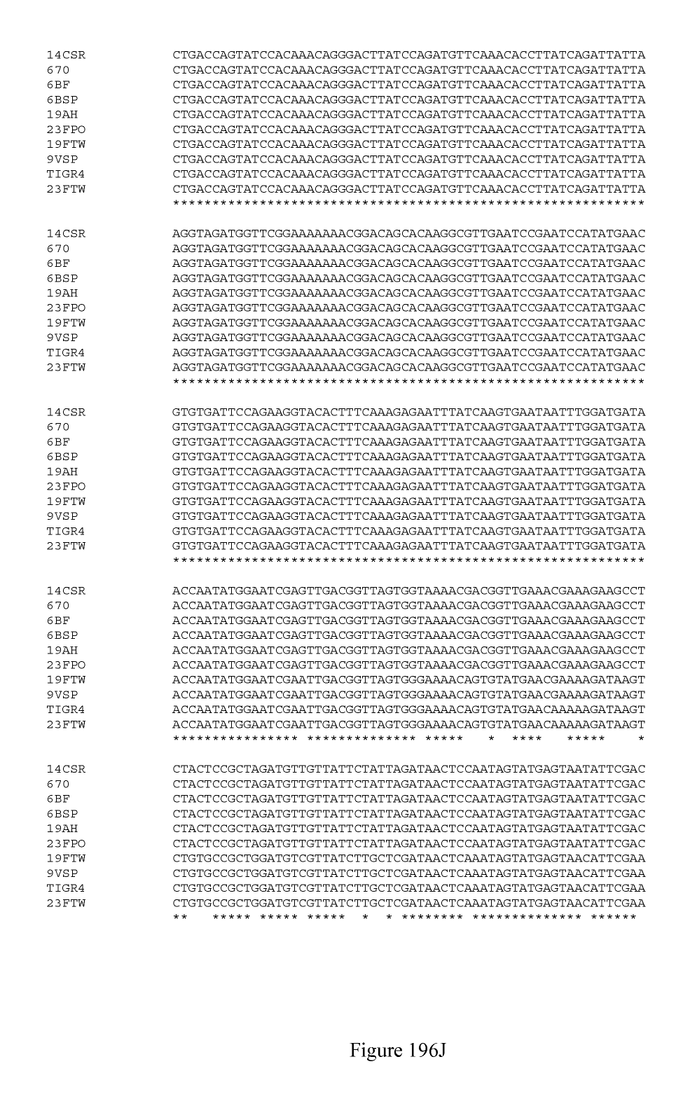

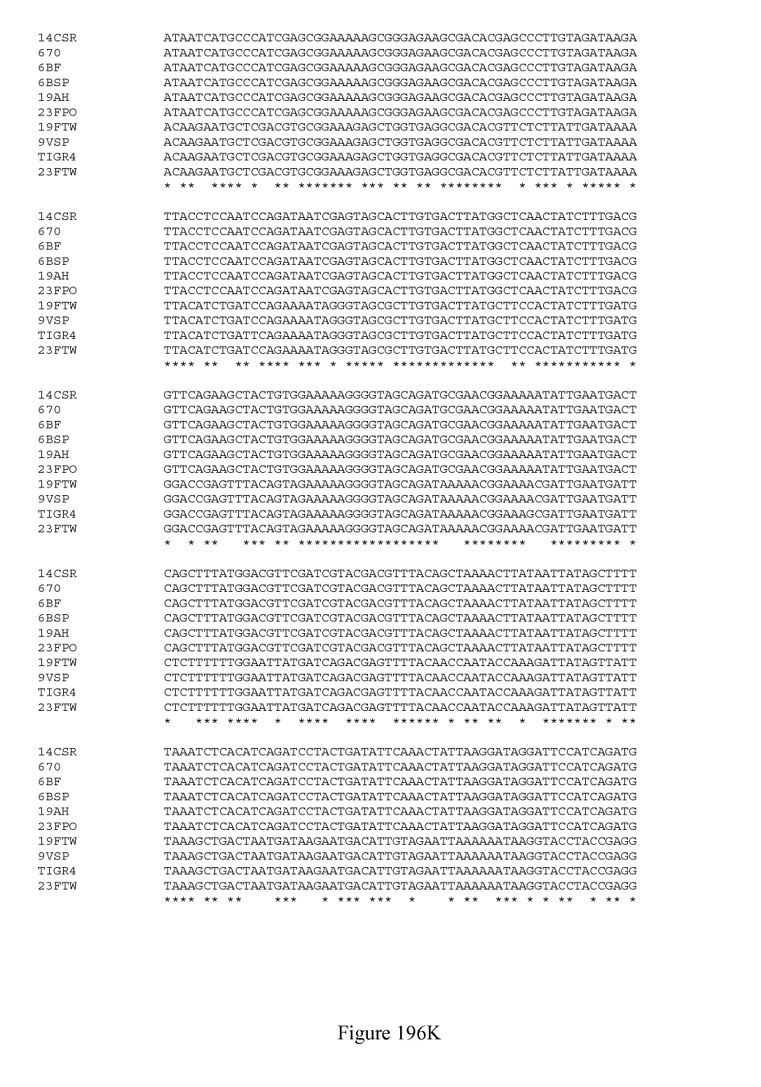

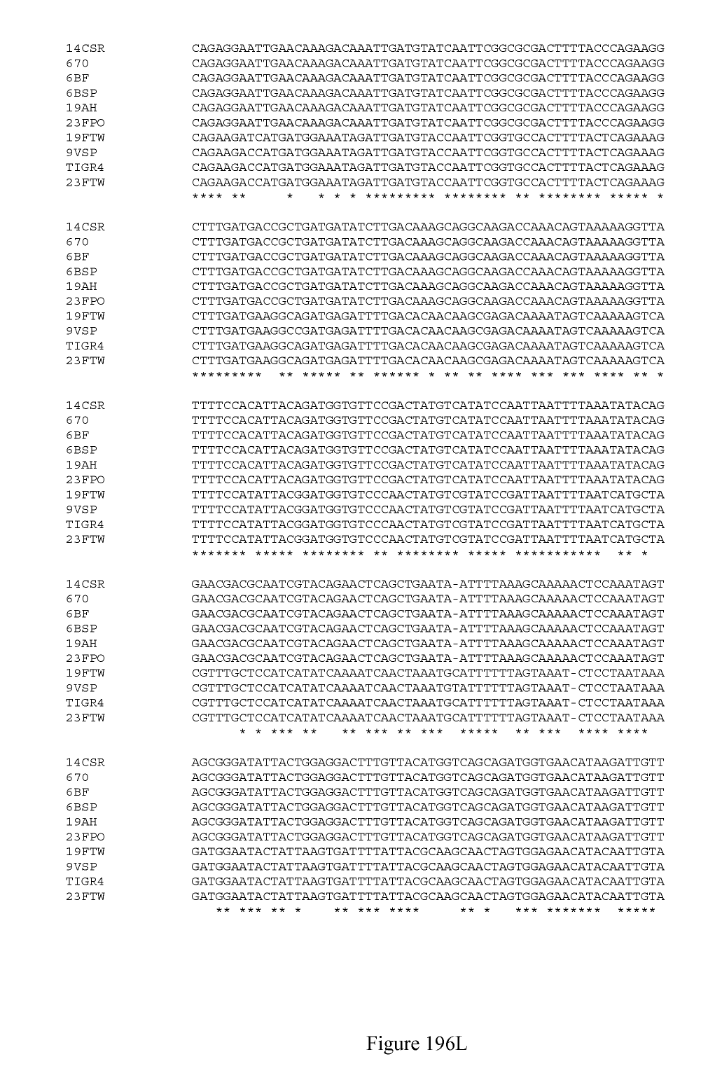

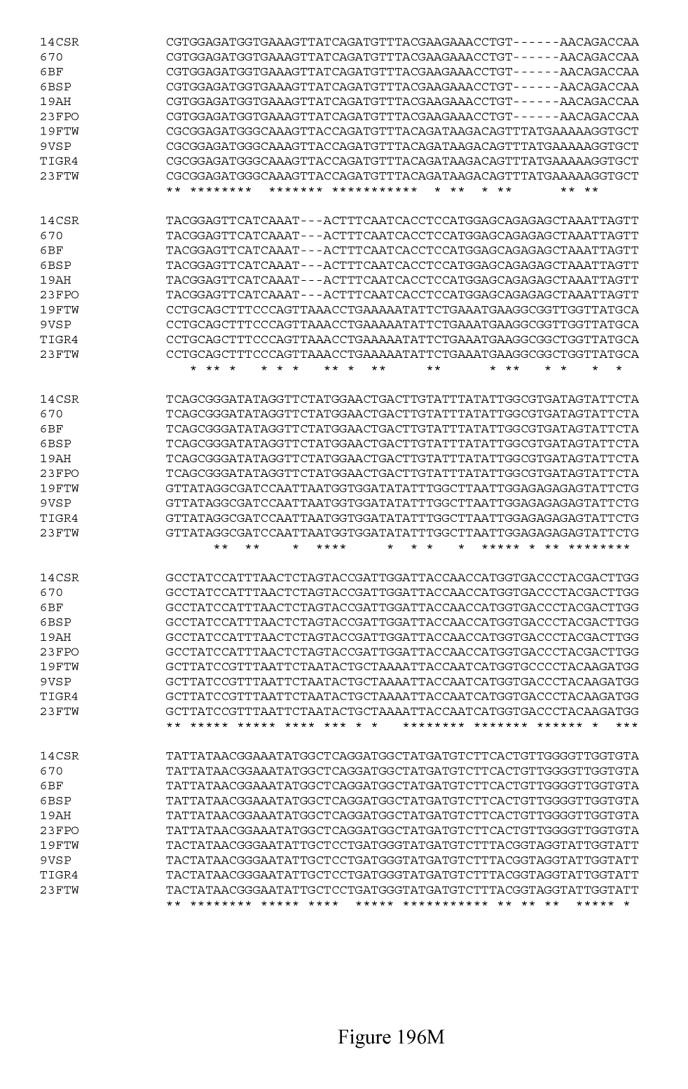

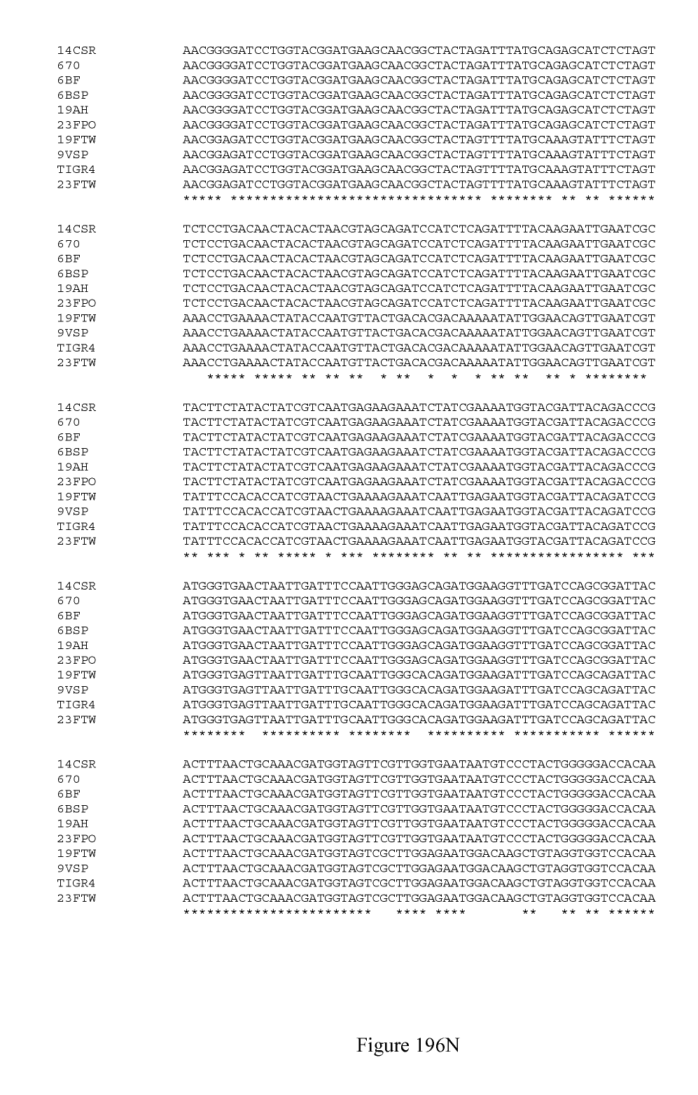

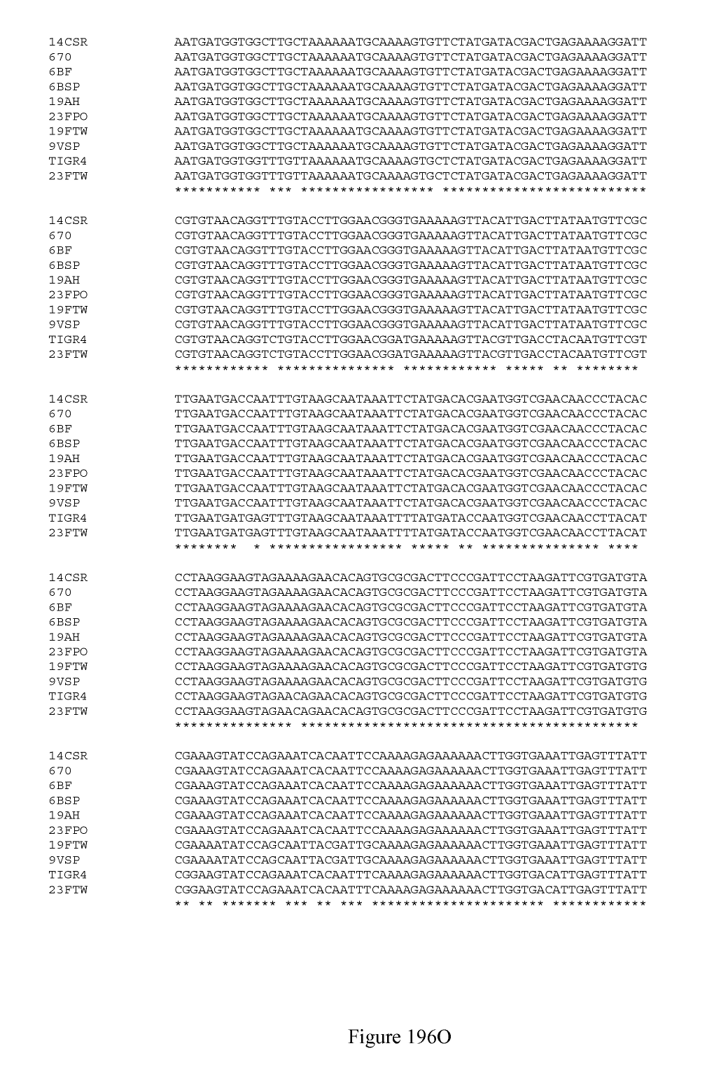

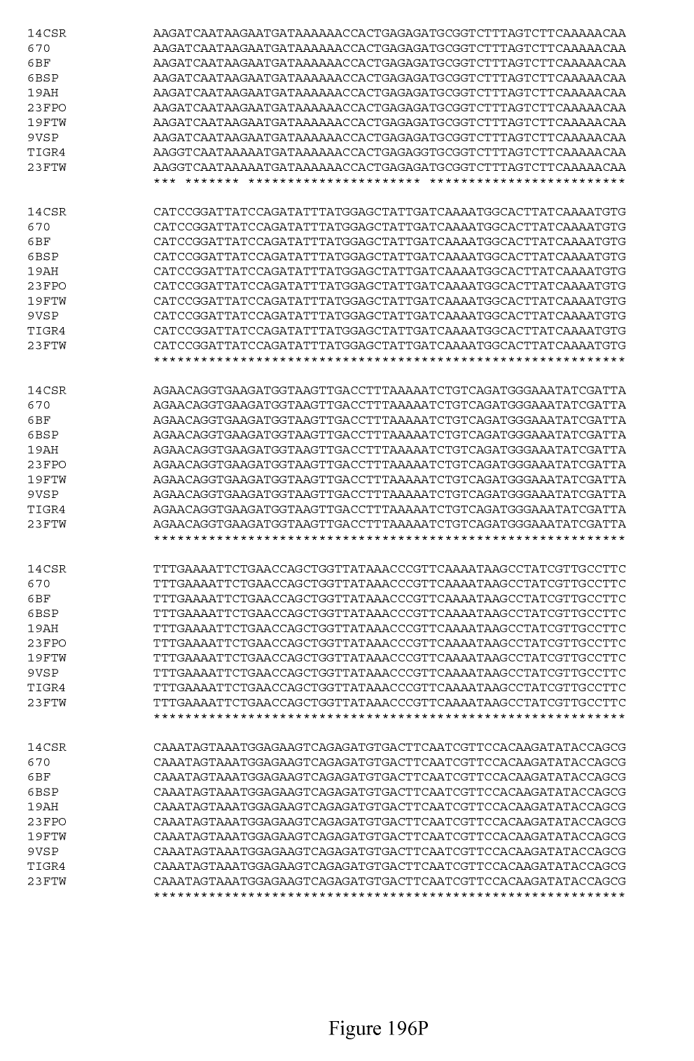

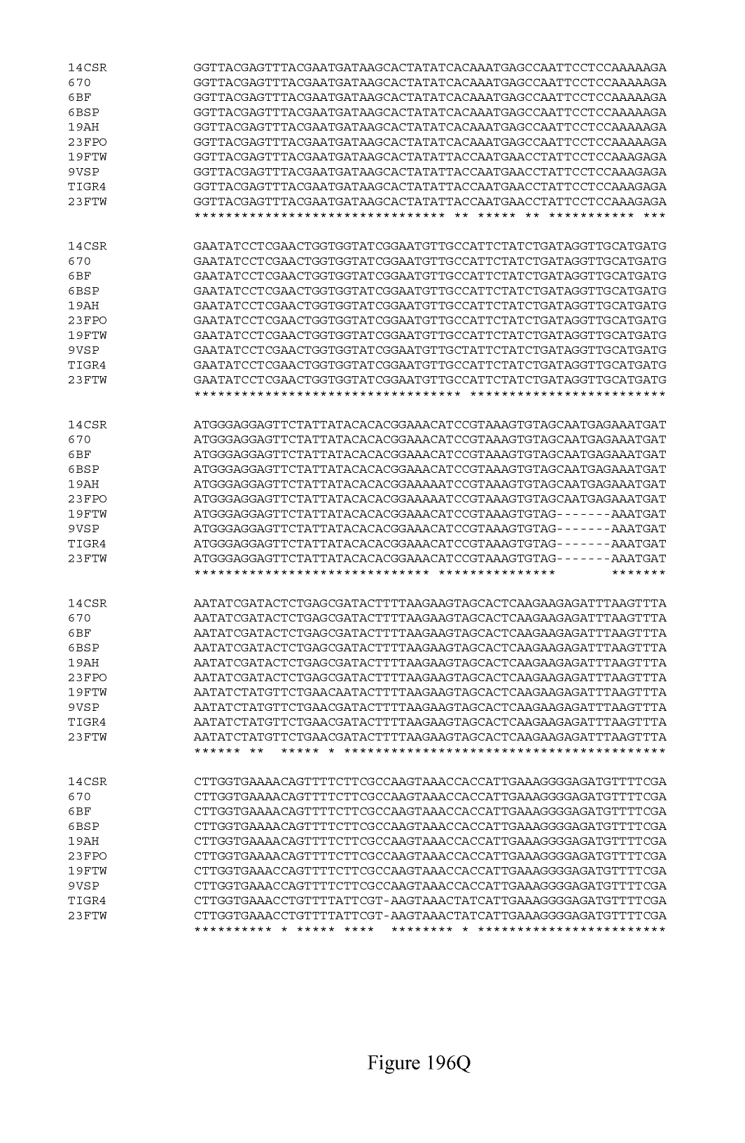

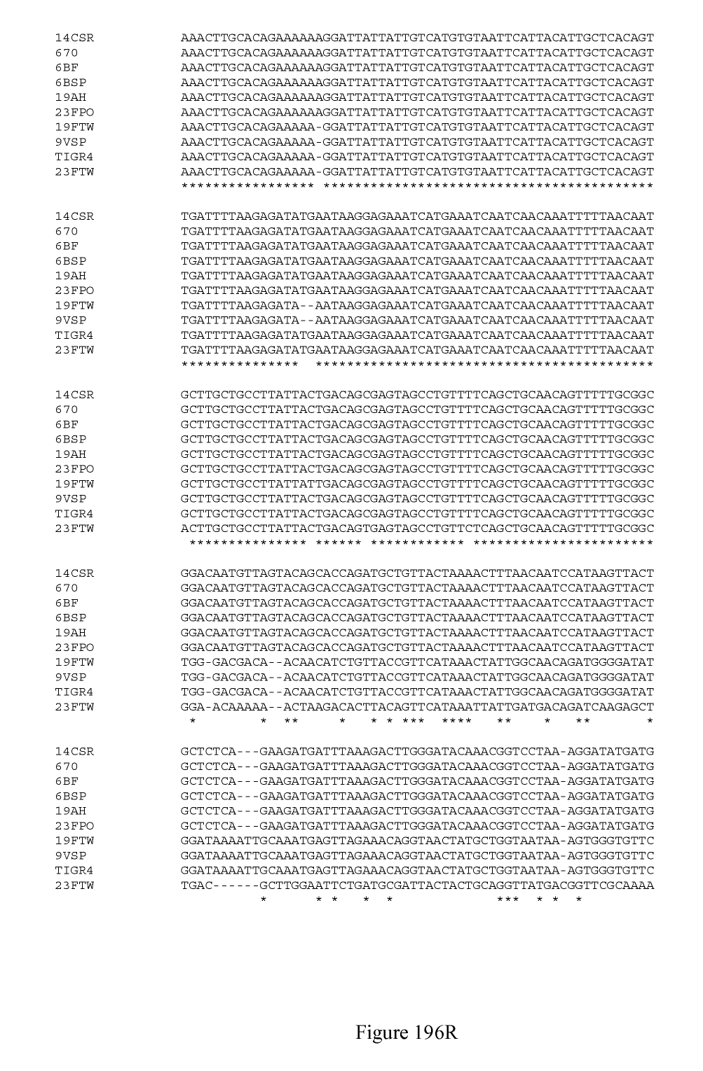

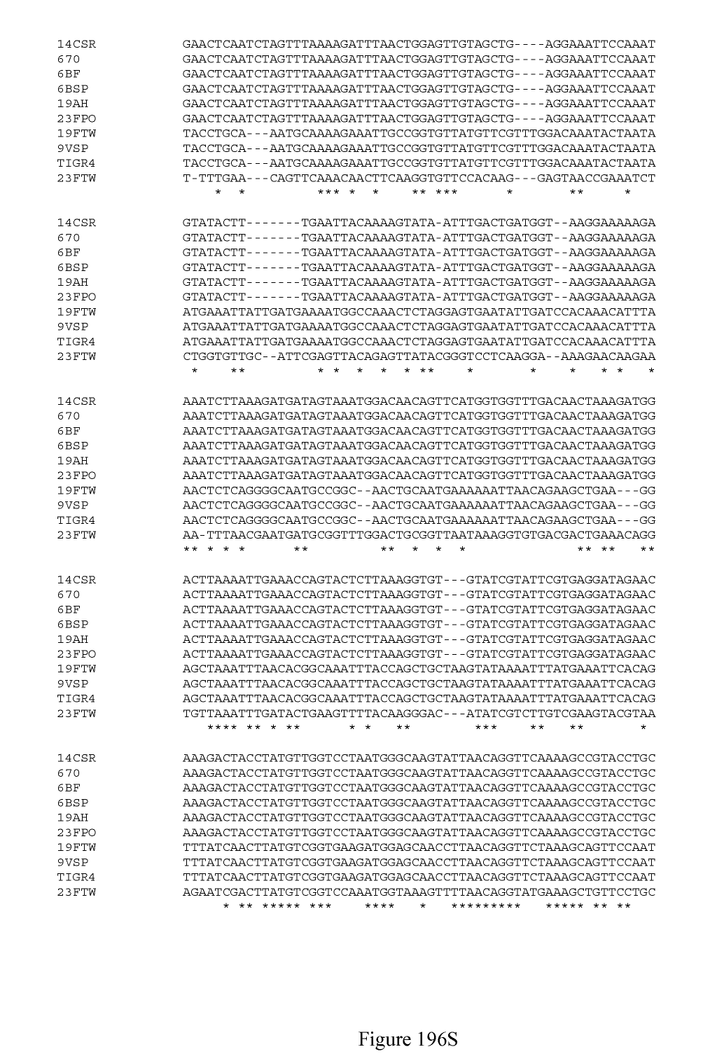

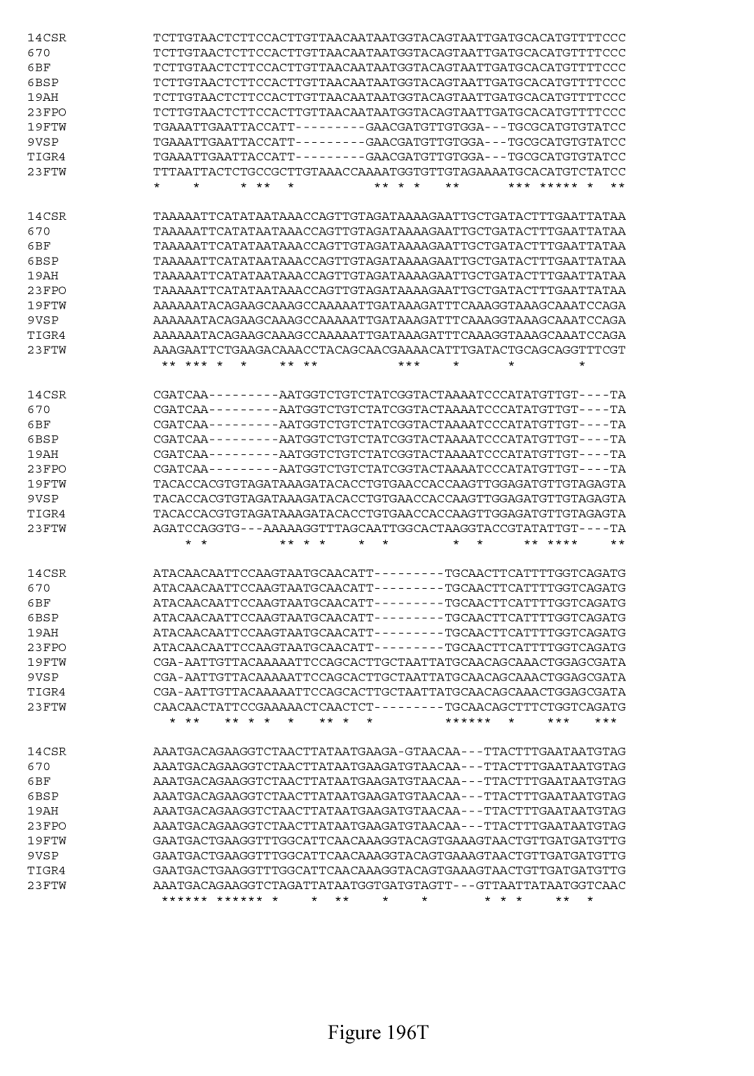

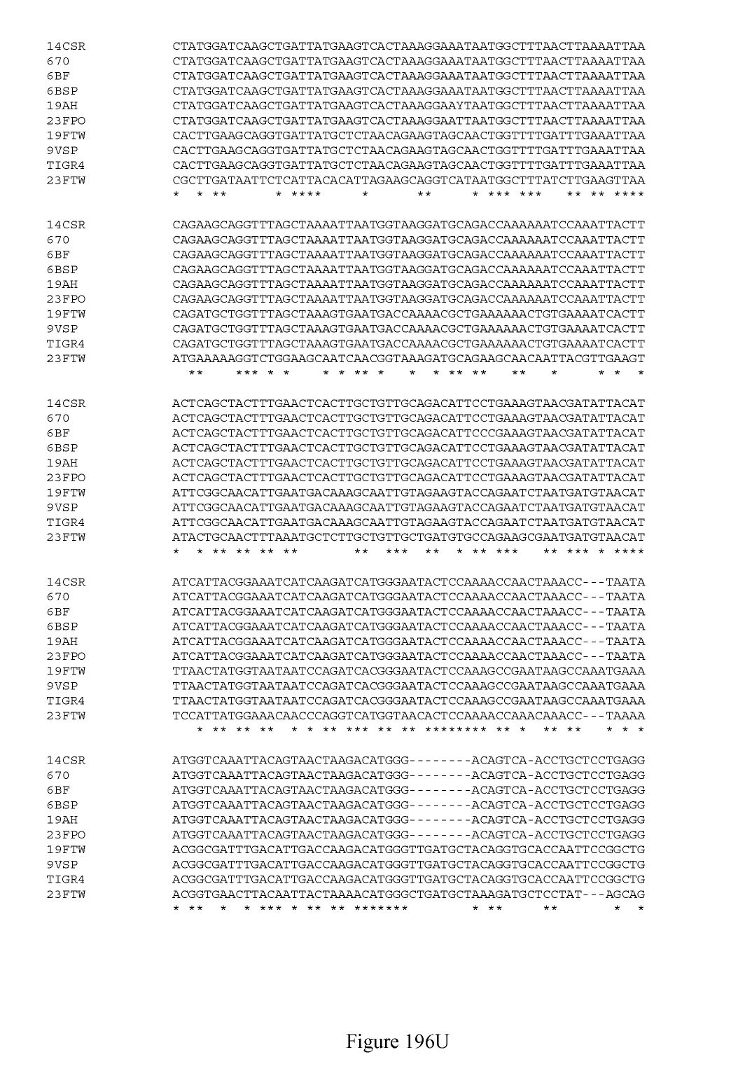

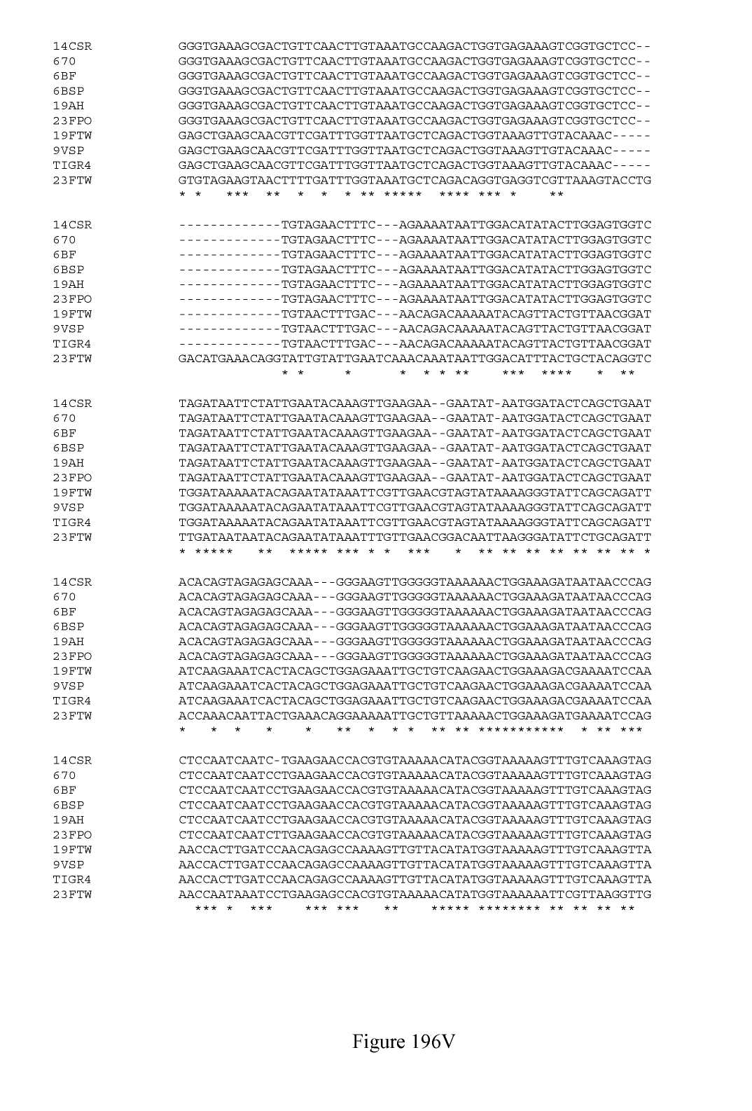

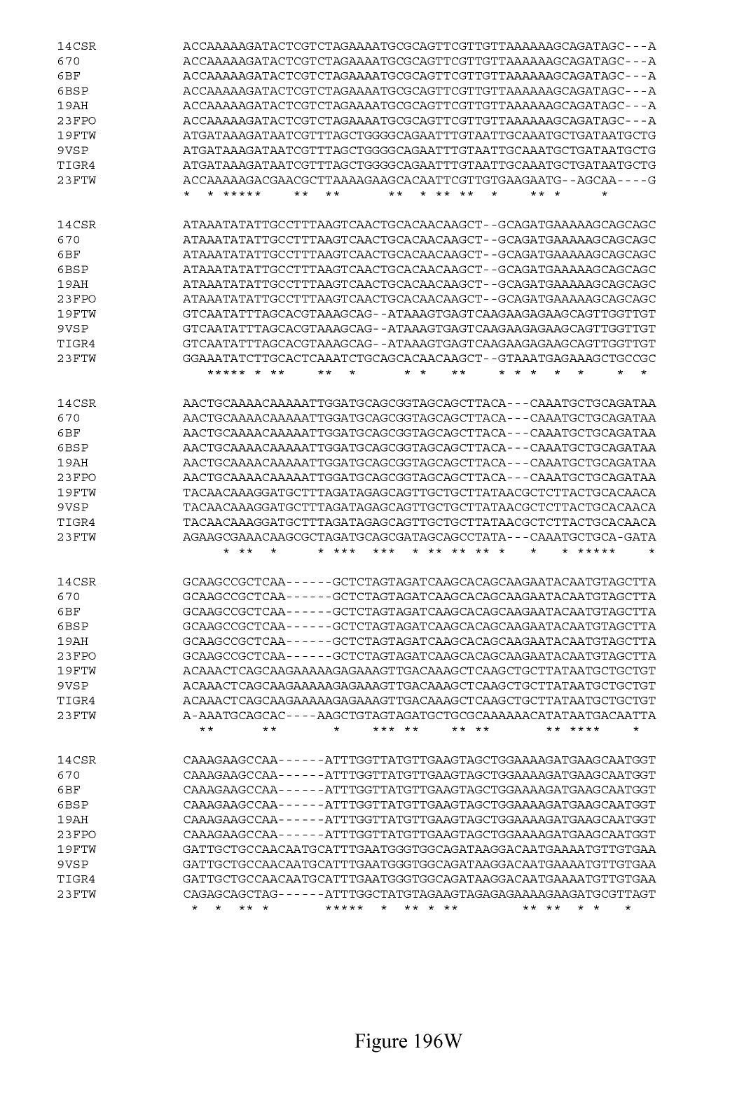

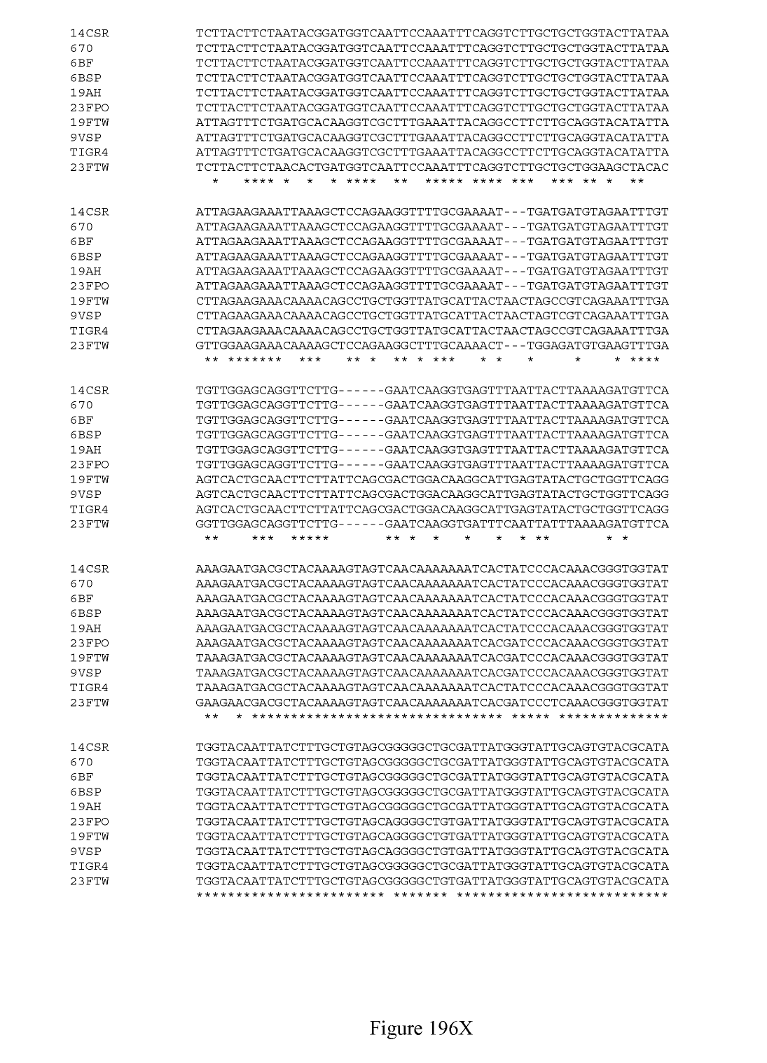

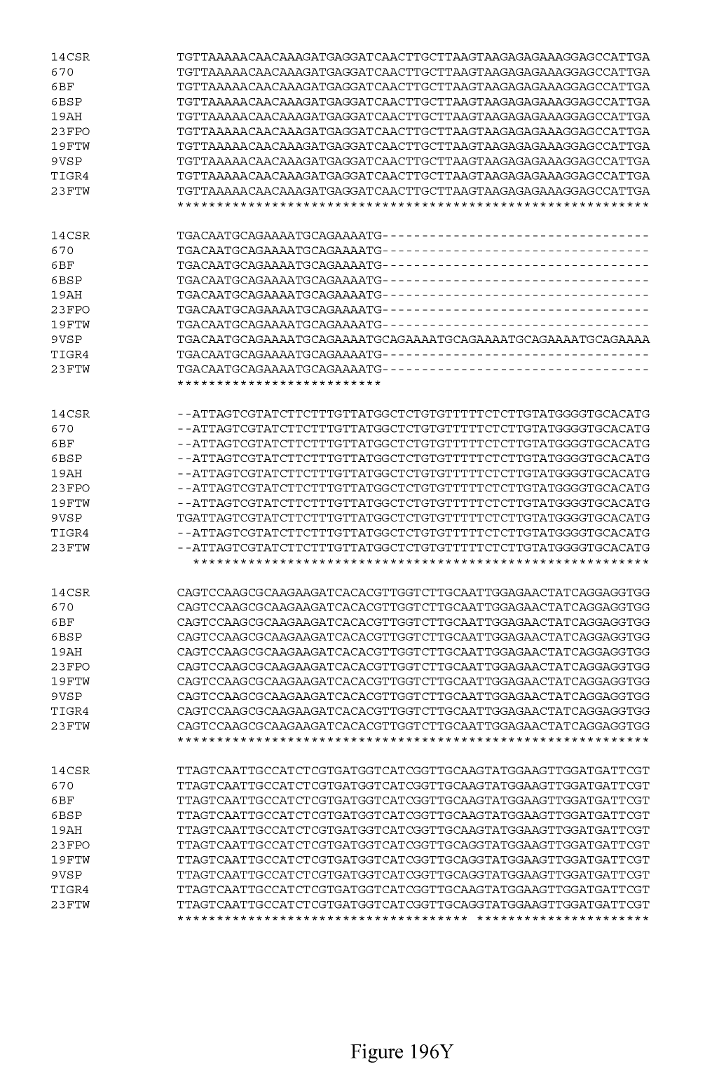

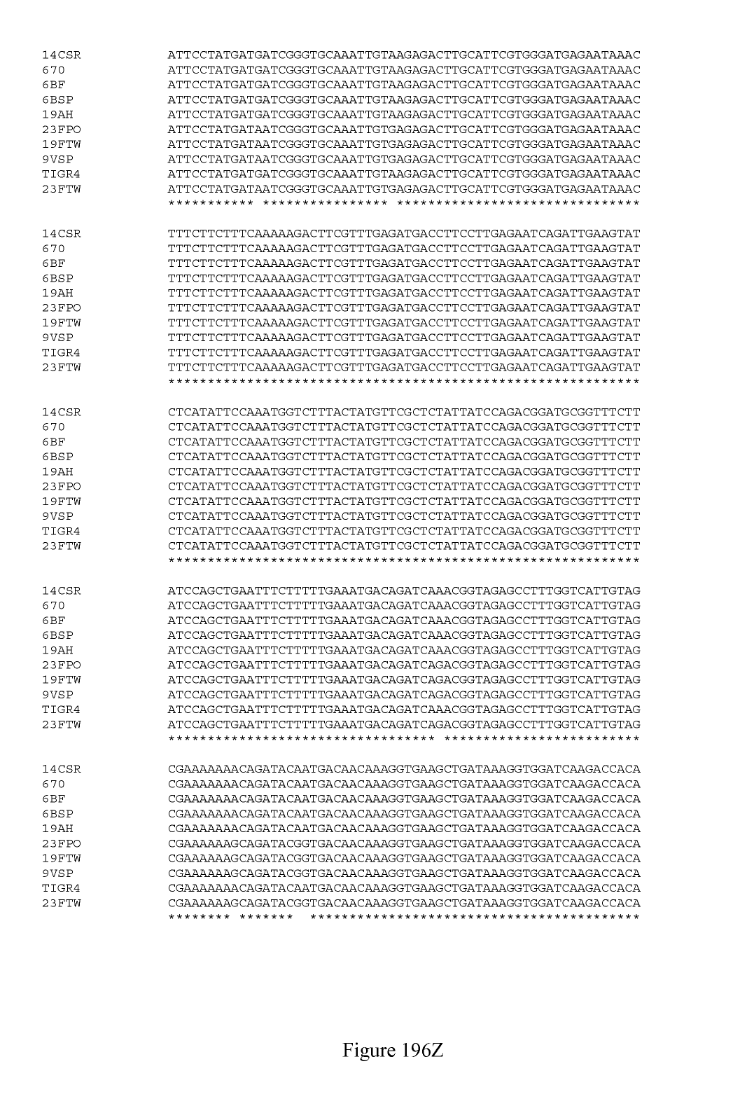

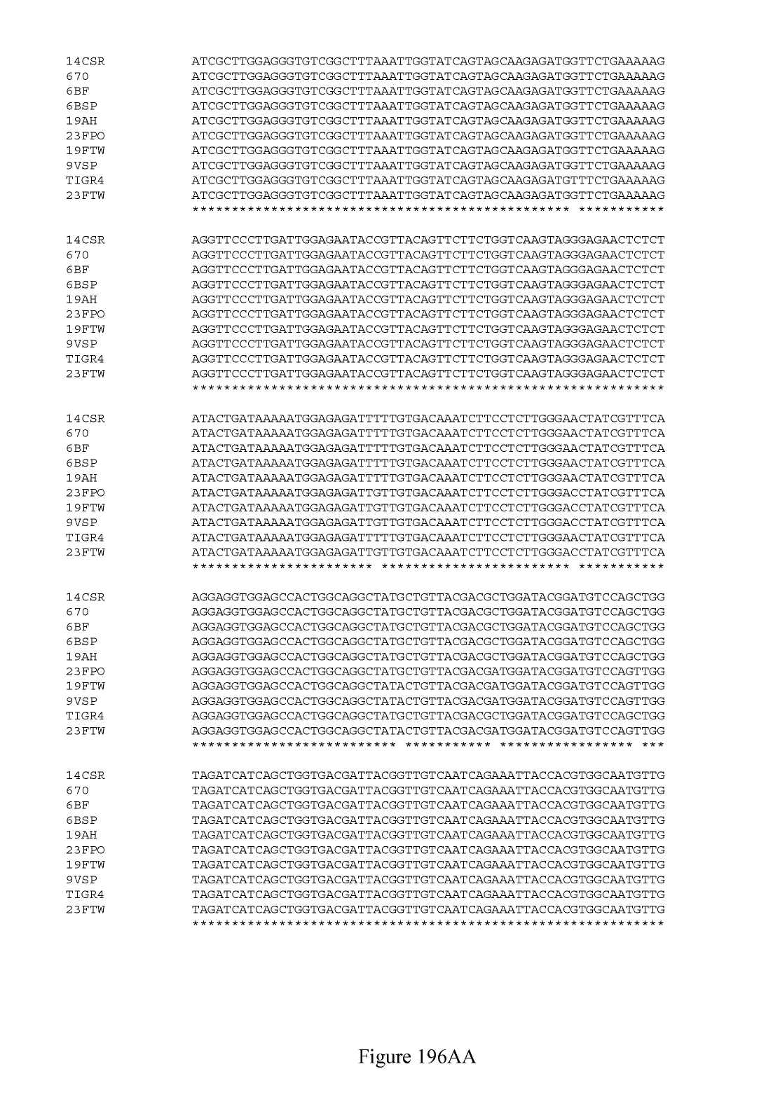

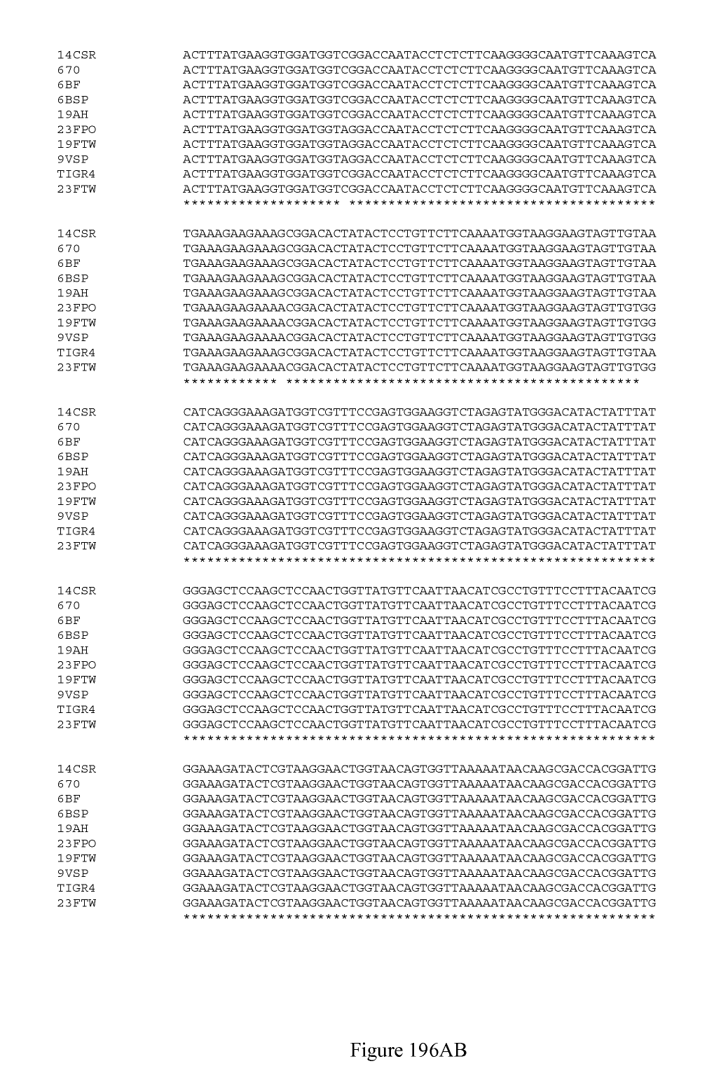

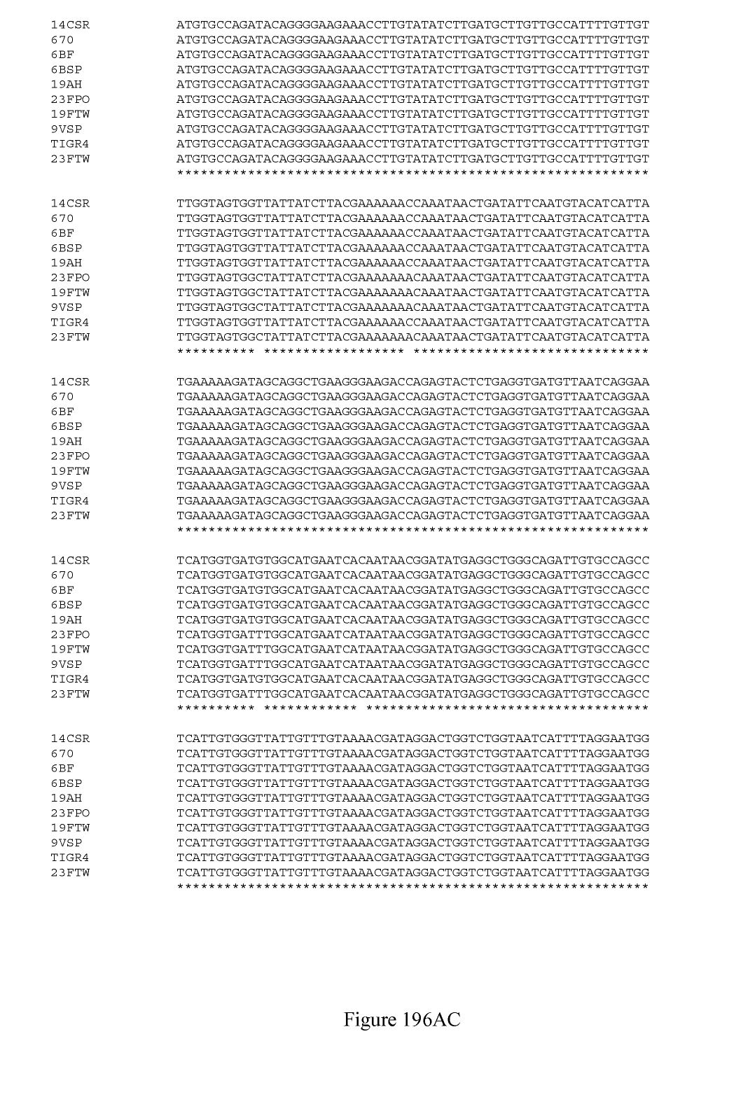

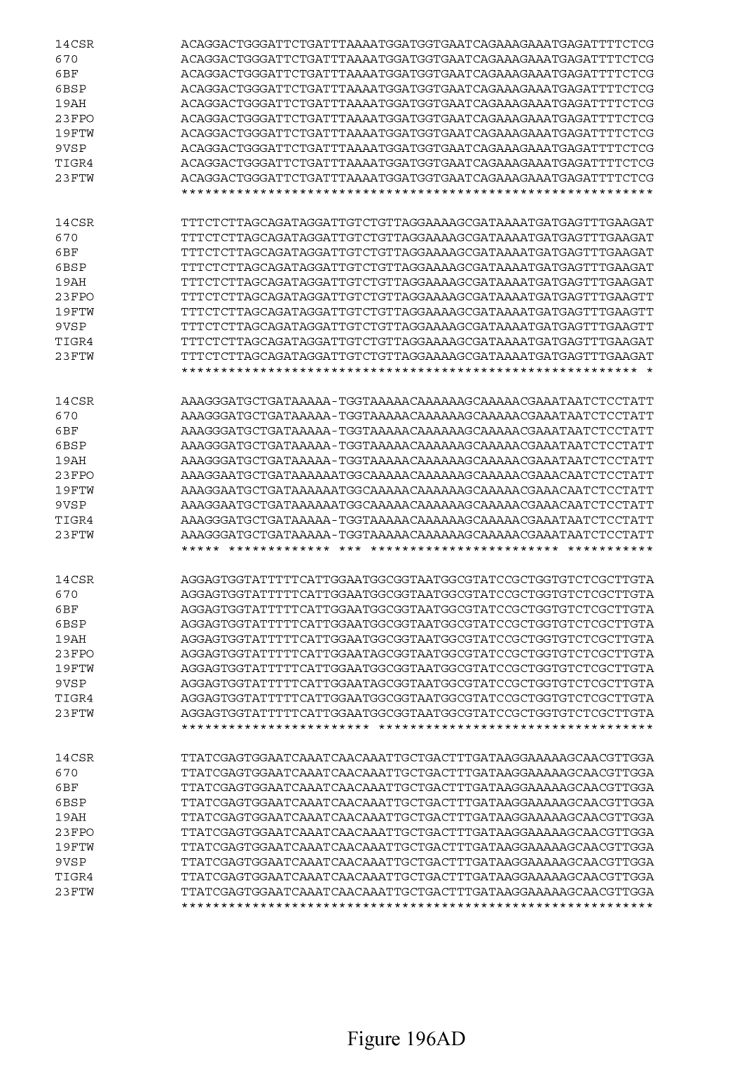

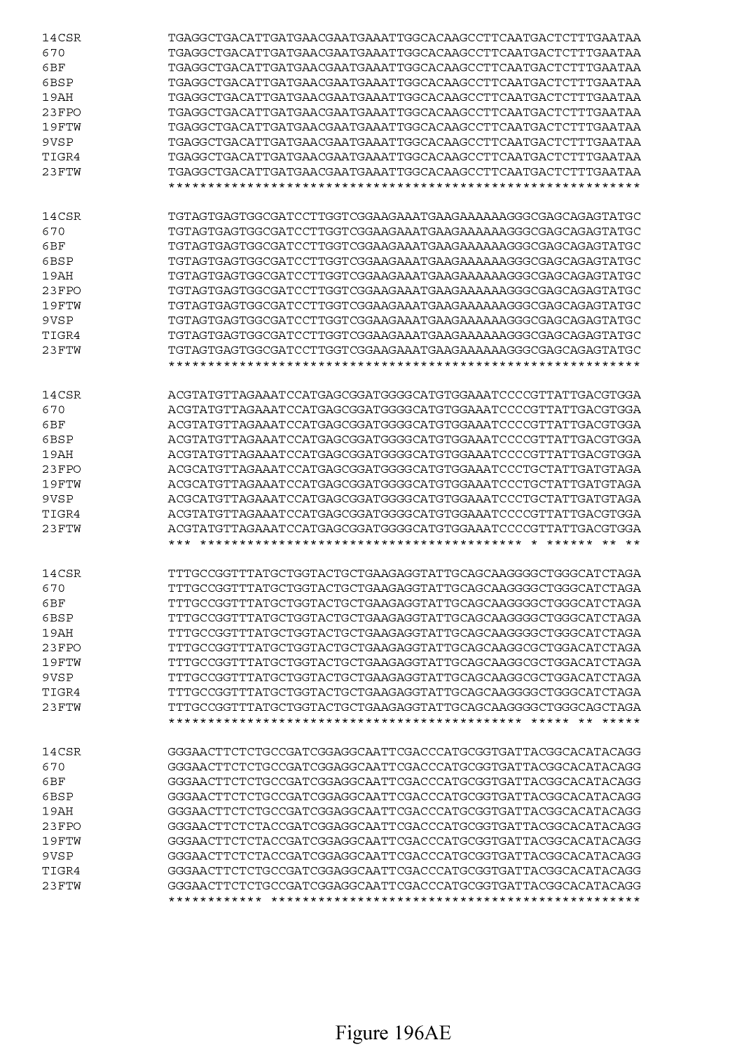

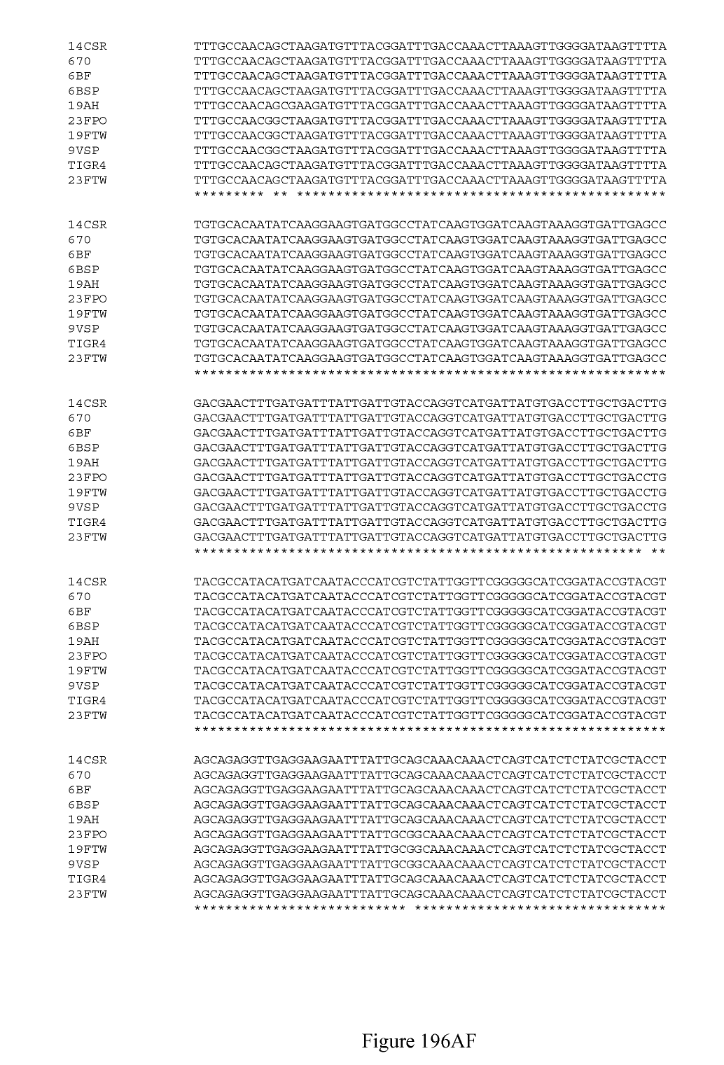

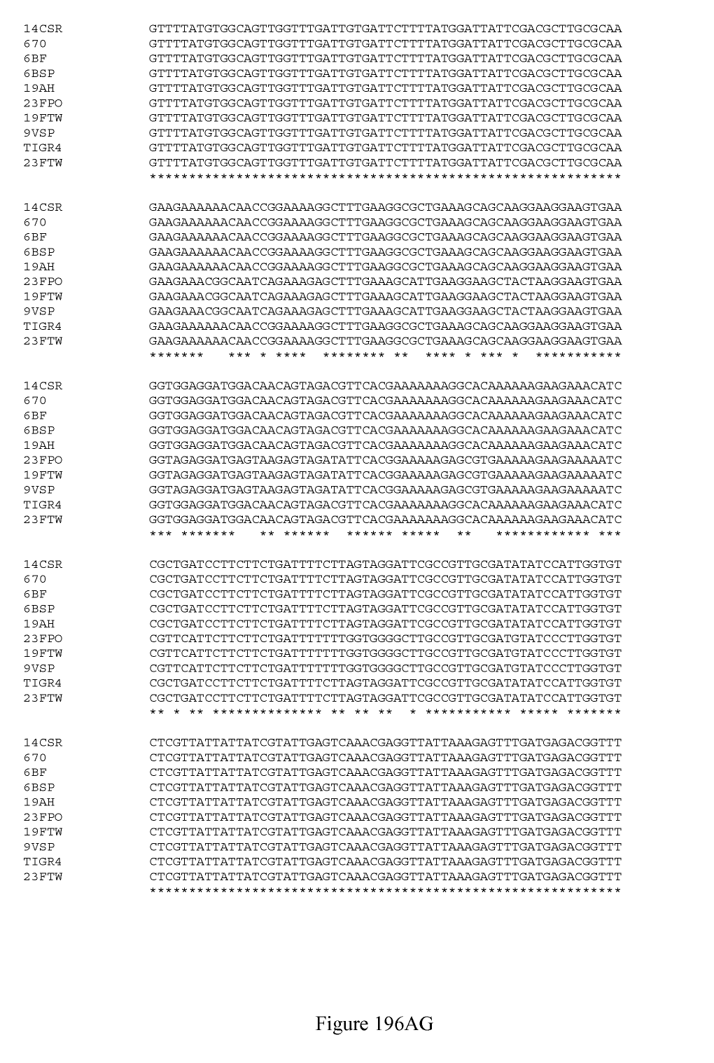

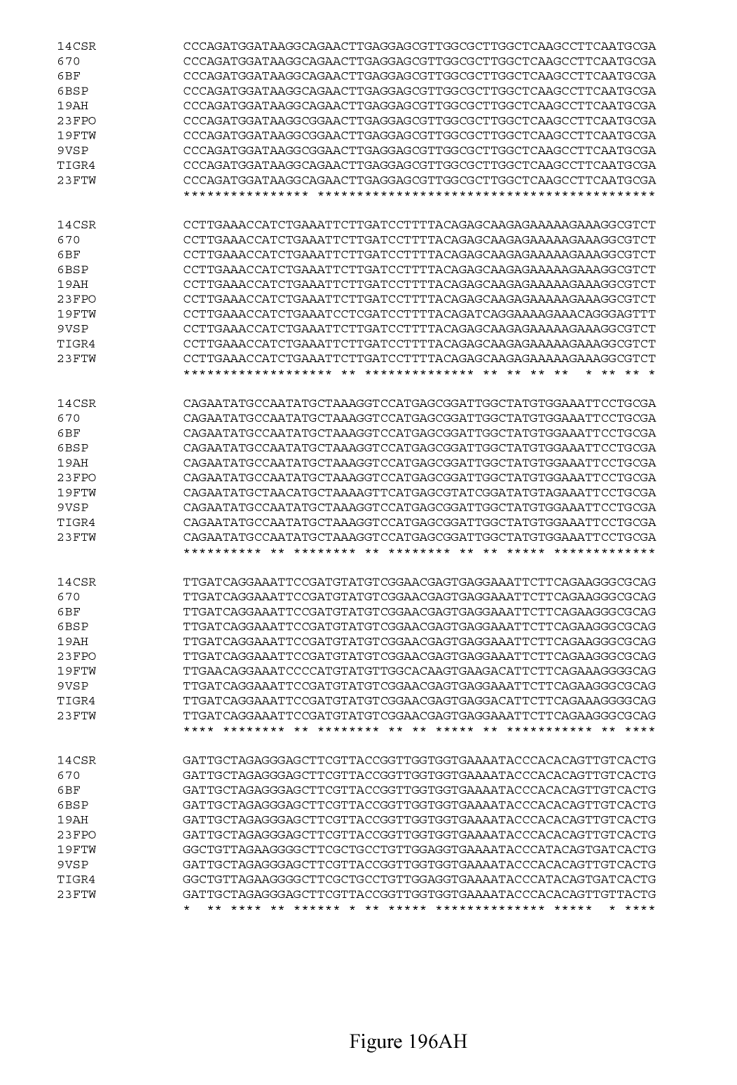

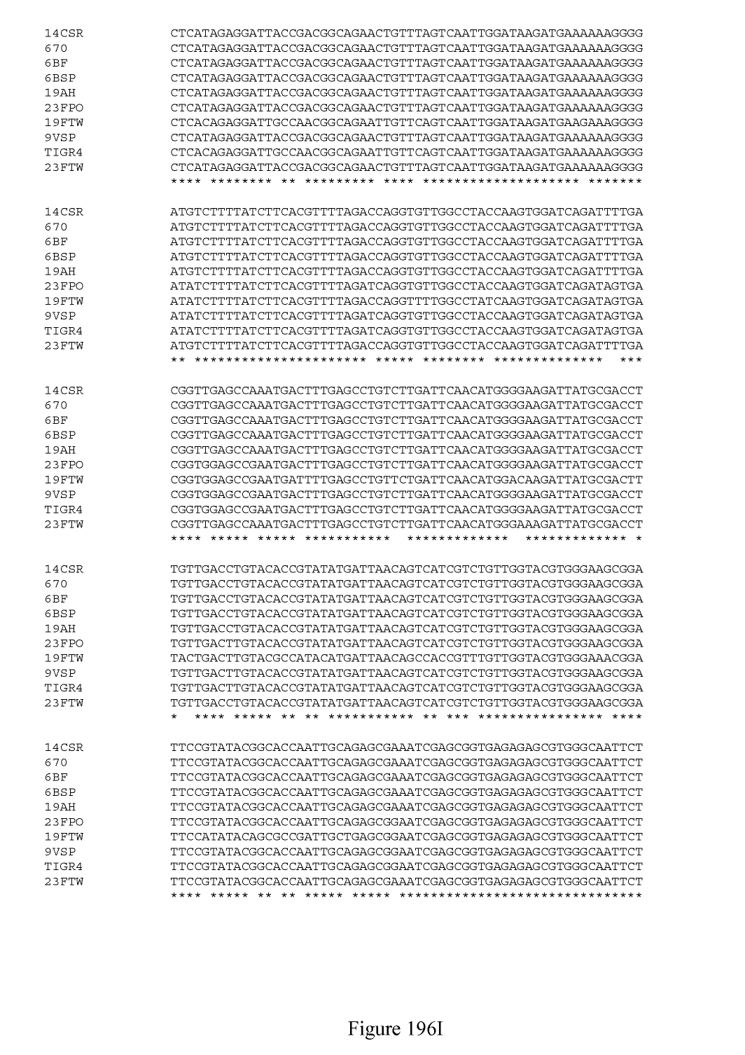

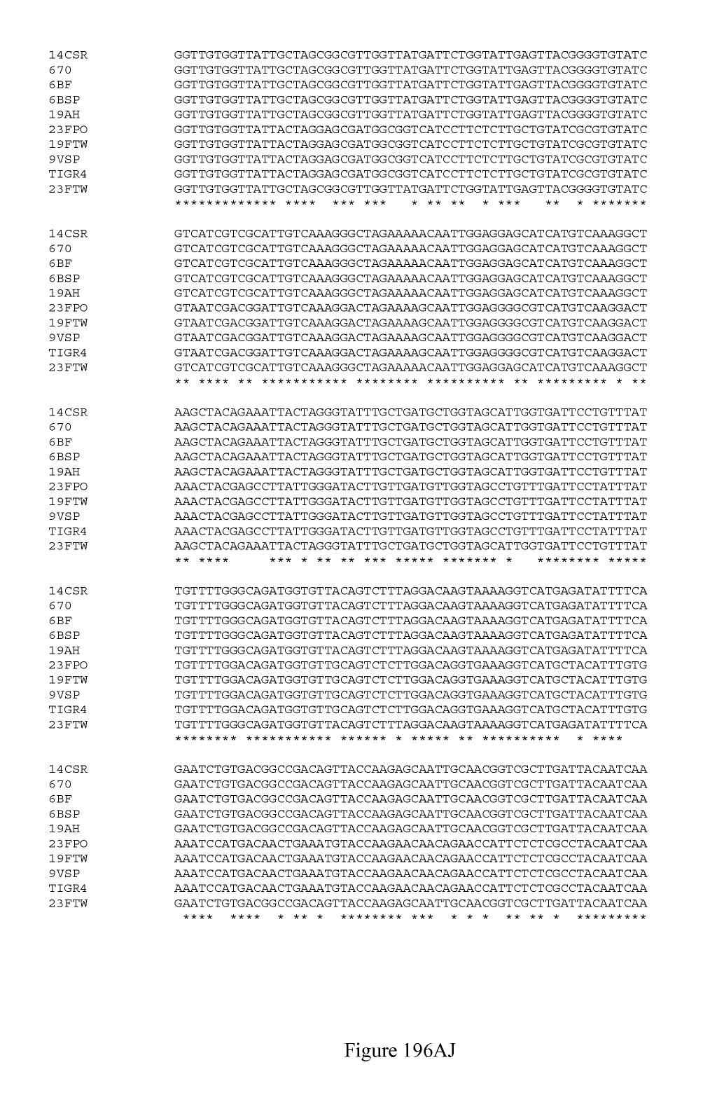

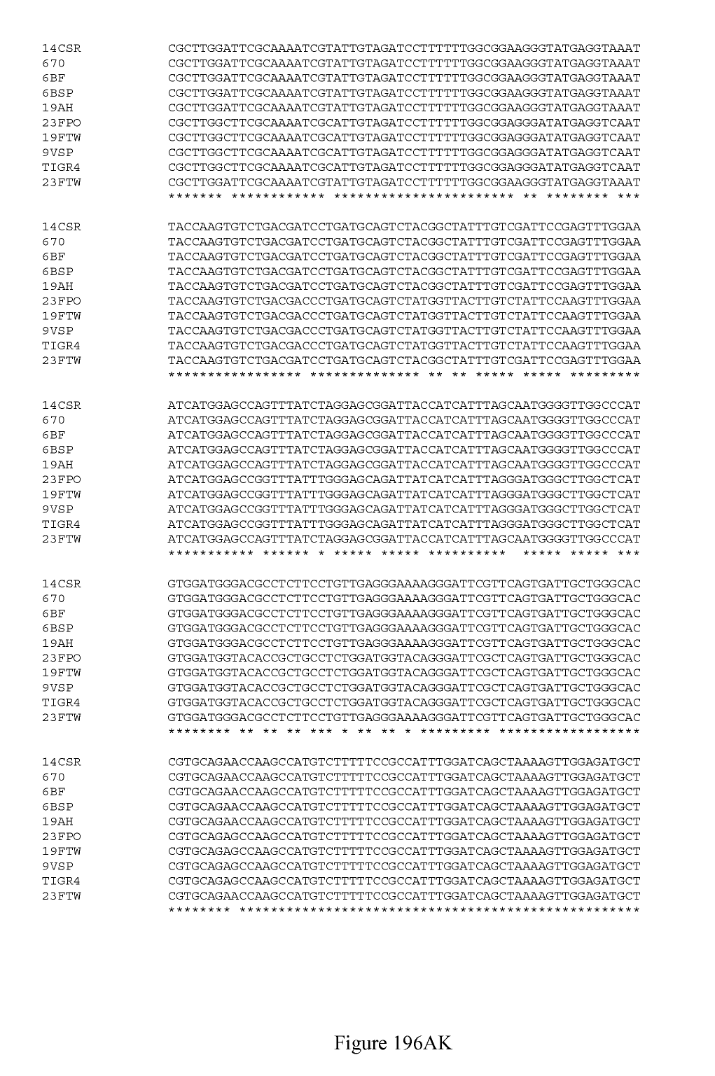

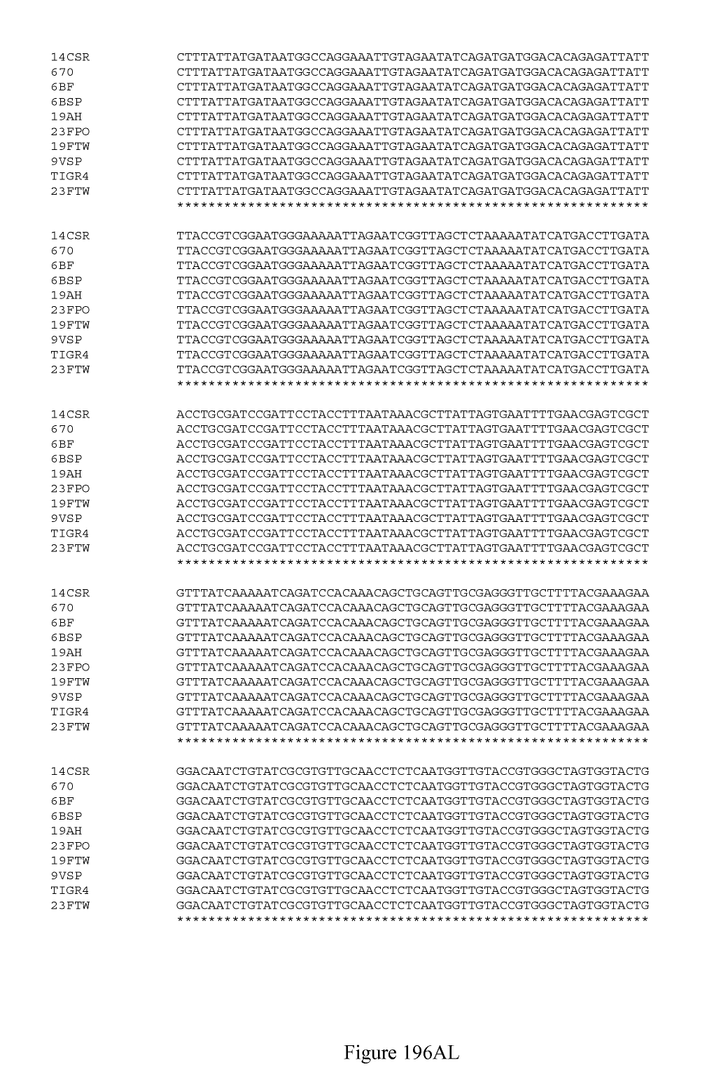

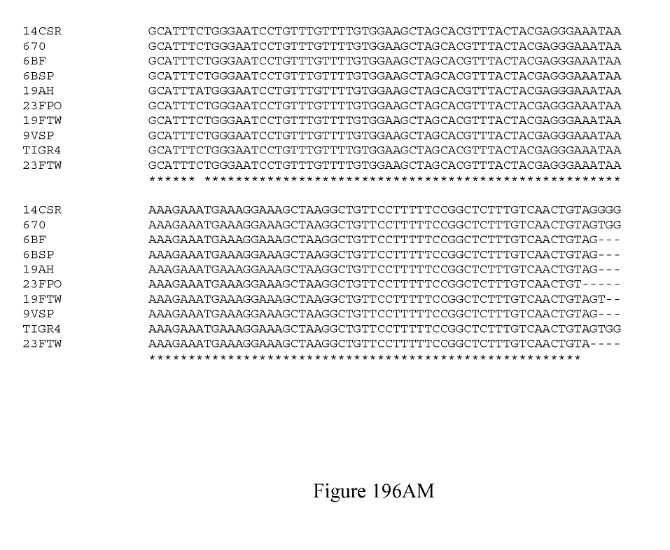

The AI locus has been sequenced for each of these strains and the nucleotide and encoded amino acid sequence for each orf has been determined An alignment of the complete nucleotide sequence of the adhesin island present in each of the ten strains is provided in FIG. 196. Aligning the amino acid sequences encoded by the orfs reveals conservation of many of the AI polypeptide amino acid sequences. For example, Table 39 provides a comparison of the percent identities of the polypeptides encoded within the S. pneumoniae strain 670 and TIGR4 adhesin islands.

TABLE 39
Percent identity comparison of
S. pneumoniae strains AI sequences

| S. pneumoniae strain | S. pneumoniae from | Shared identity |
|---|---|---|
| 670 polypeptide | TIGR4 polypeptide | of polypeptides |
| Orf1_670 | SP0460 | 99.3% identity in |
| | | 422 aa overlap |
| Orf2_670 | SP0461 | 100.0% identity in |
| | | 509 aa overlap |
| Orf3_670 | SP0462 | 83.2% identity in |
| | | 895 aa overlap |
| Orf4_670 | SP0463 | 47.9% identity in |
| | | 678 aa overlap |
| Orf5_670 | SP0464 | 99.7% identity in |
| | | 393 aa overlap |
| Orf6_670 | SP0466 | 100.0% identity in |
| | | 279 aa overlap |
| Orf7_670 | SP0467 | 94.2% identity in |
| | | 260 aa overlap |
| Orf8_670 | SP0468 | 91.5% identity in |
| | | 283 aa overlap |

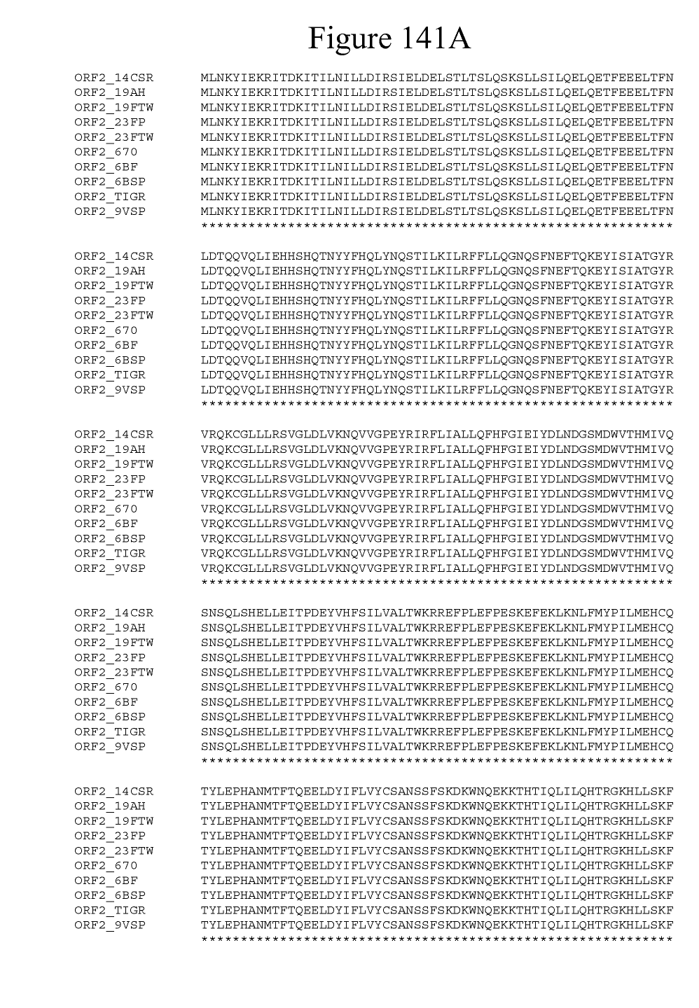

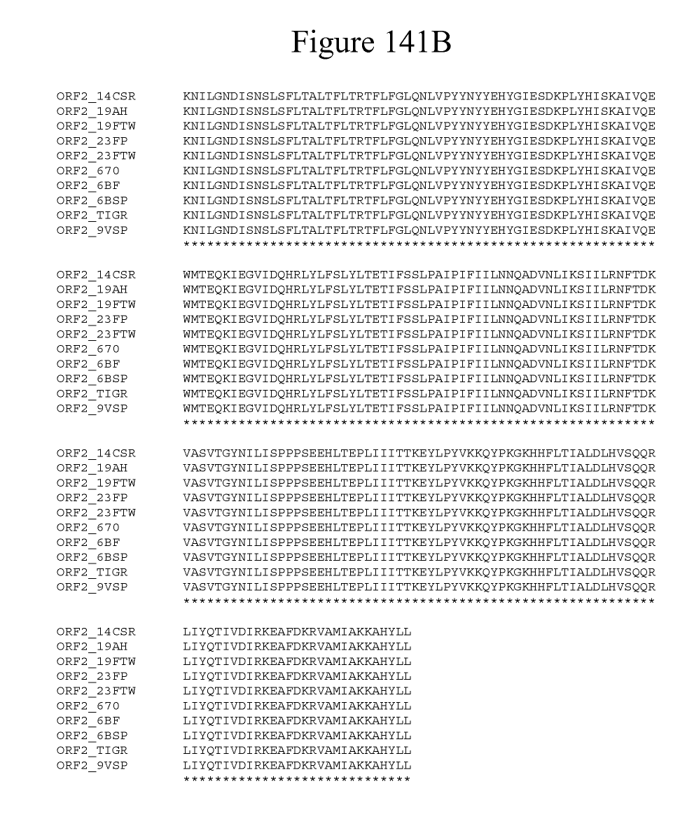

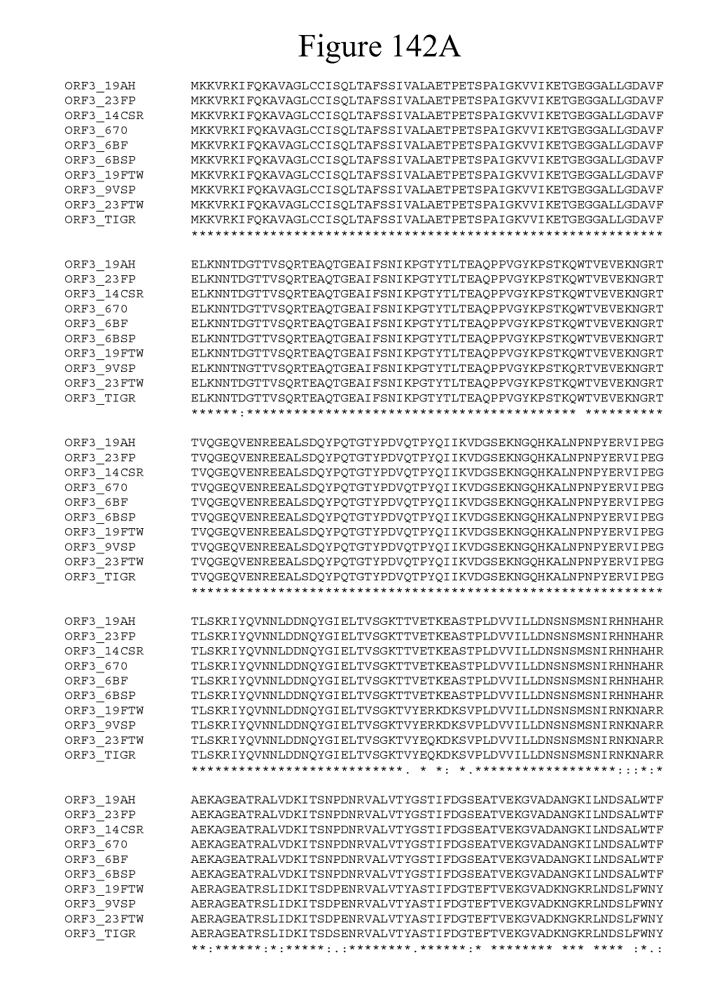

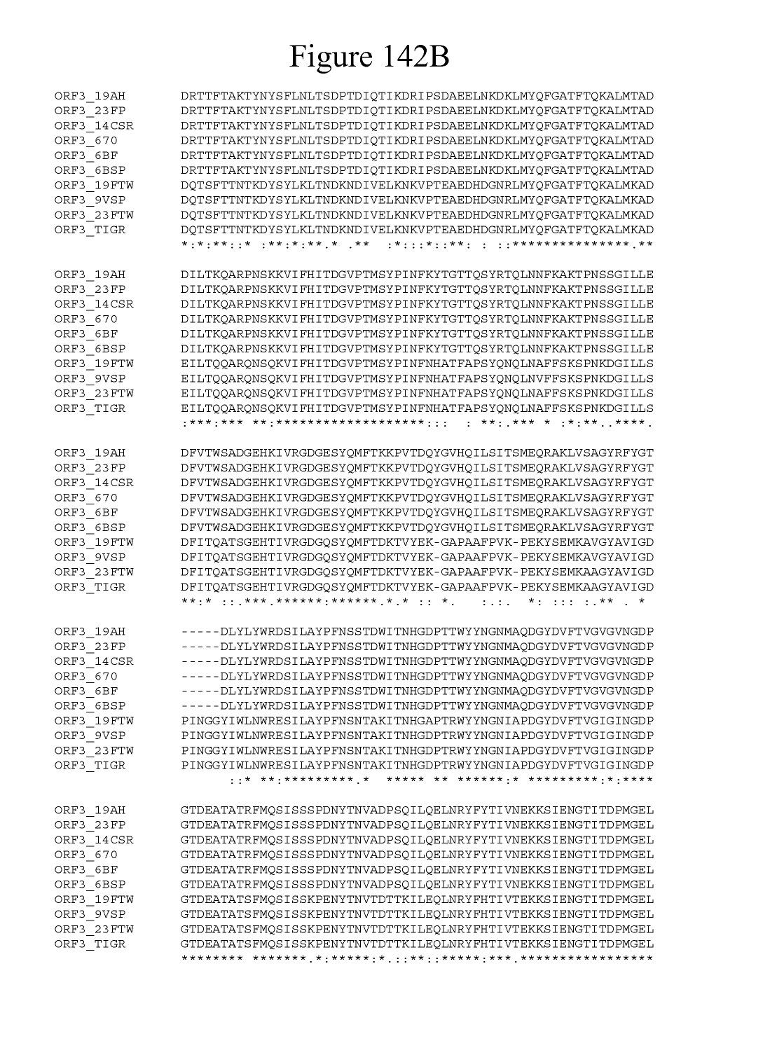

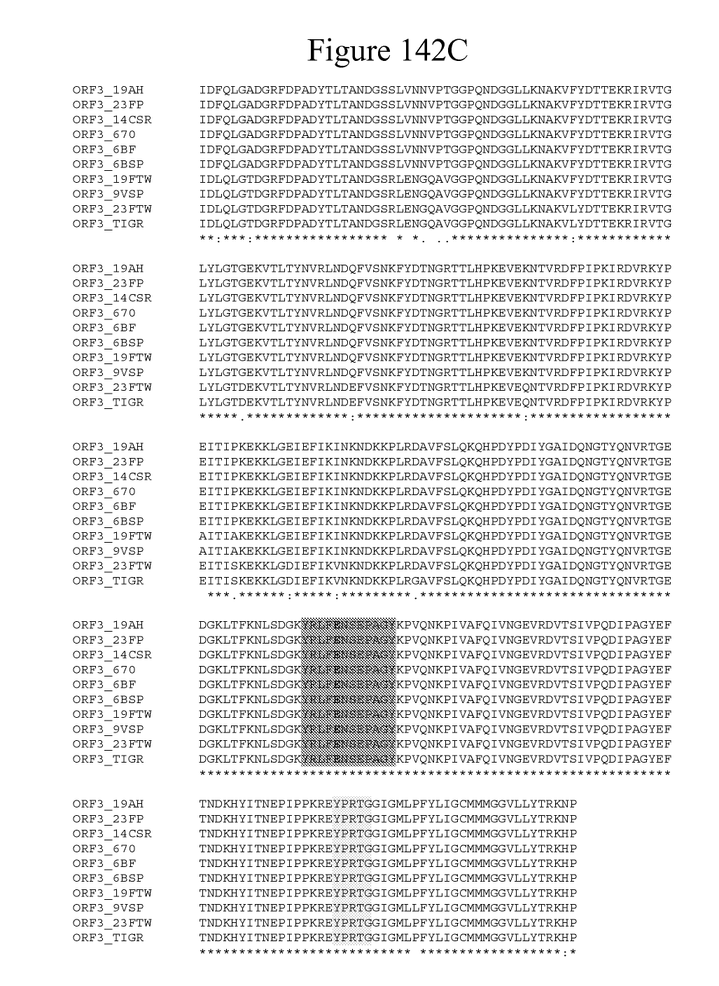

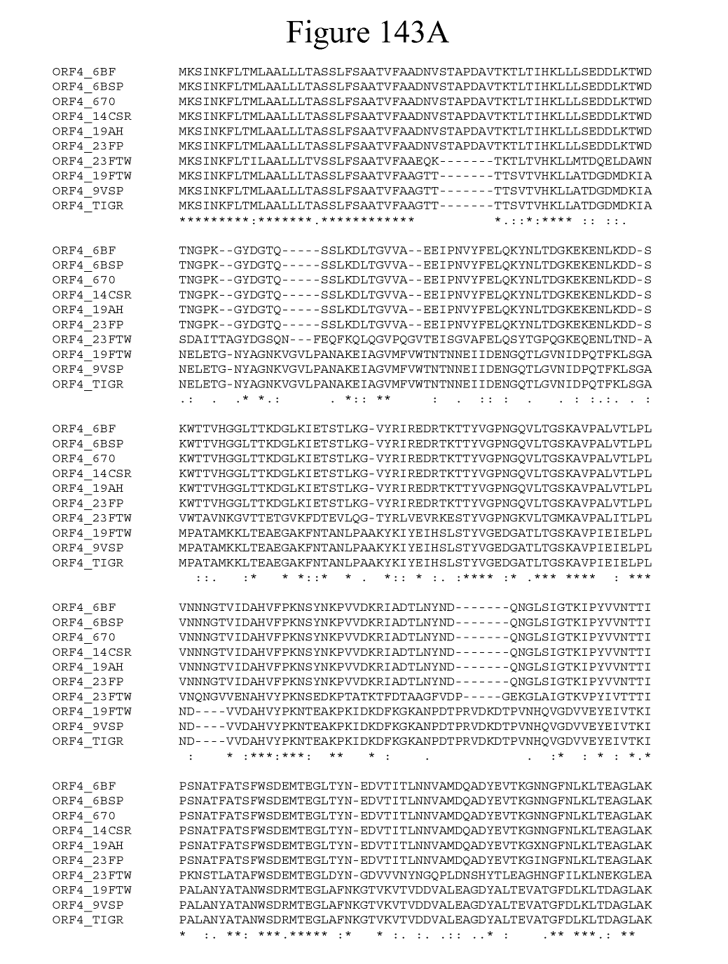

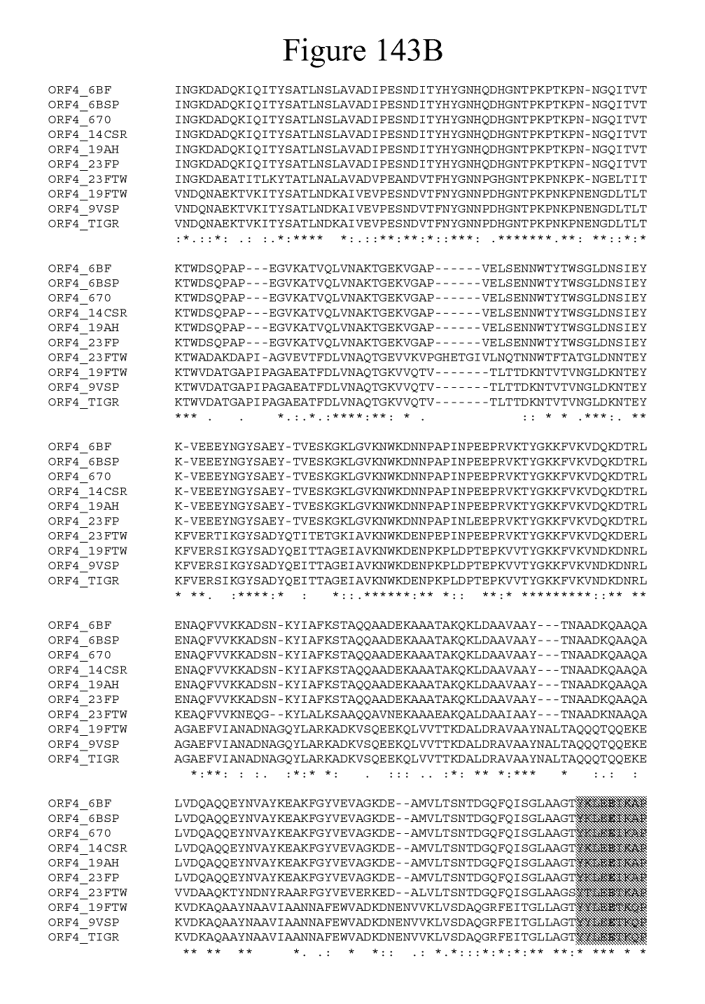

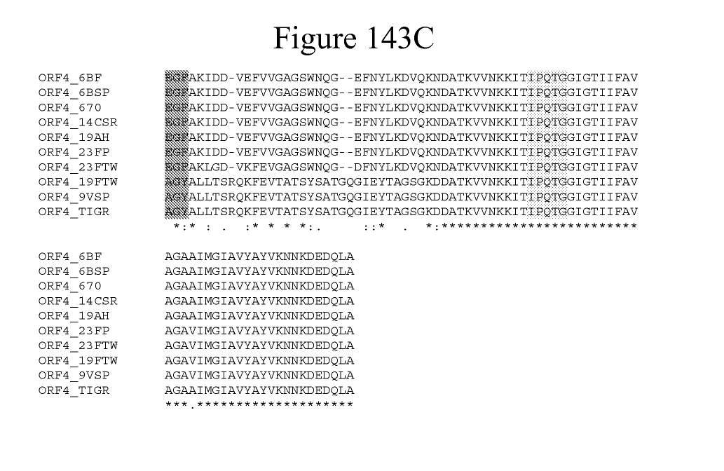

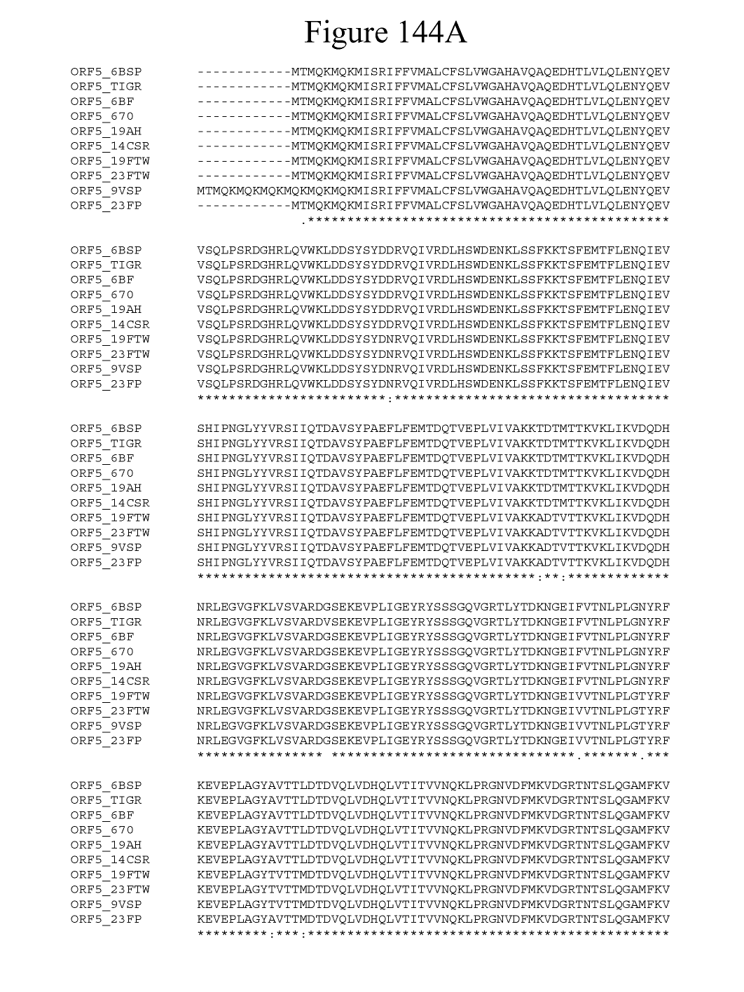

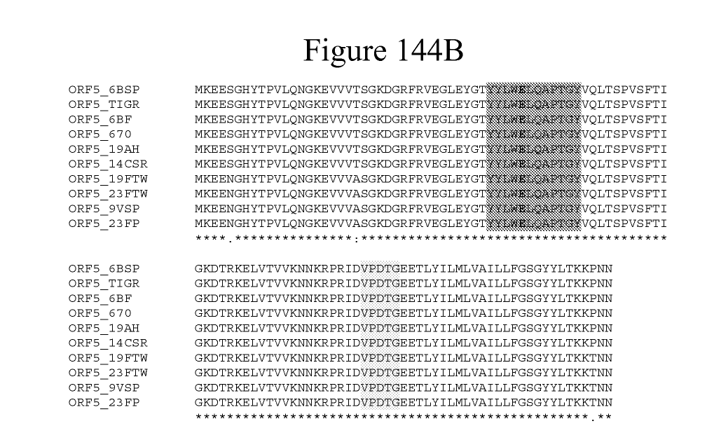

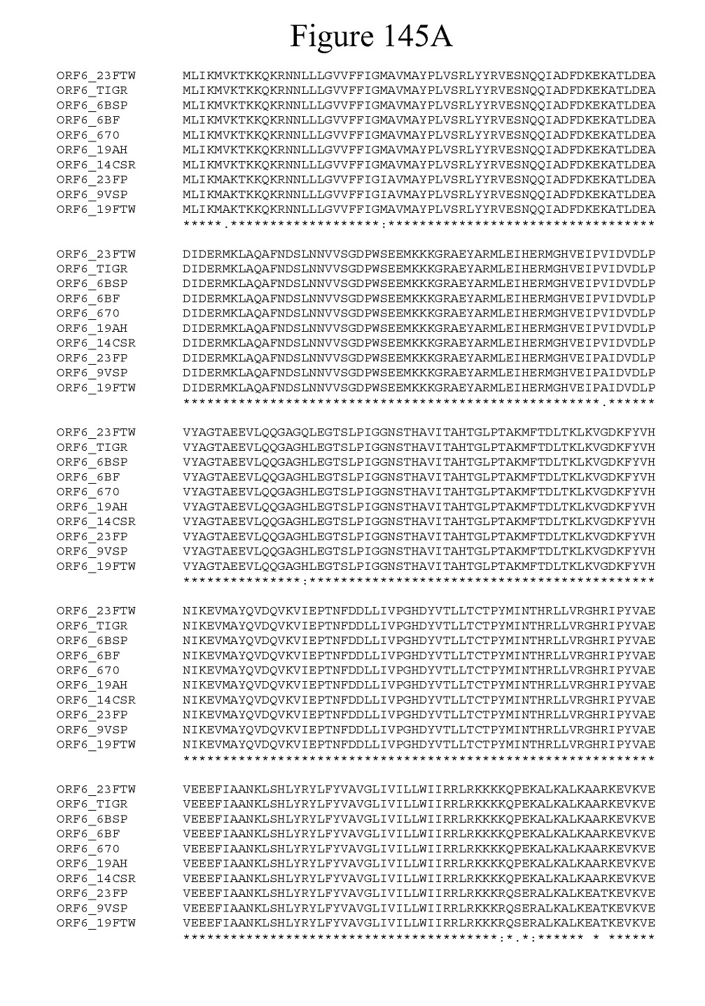

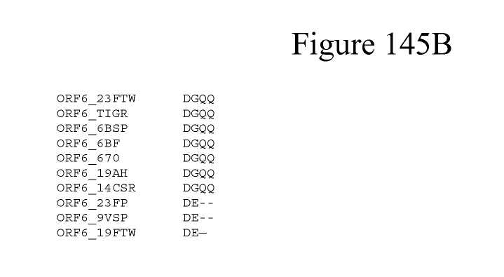

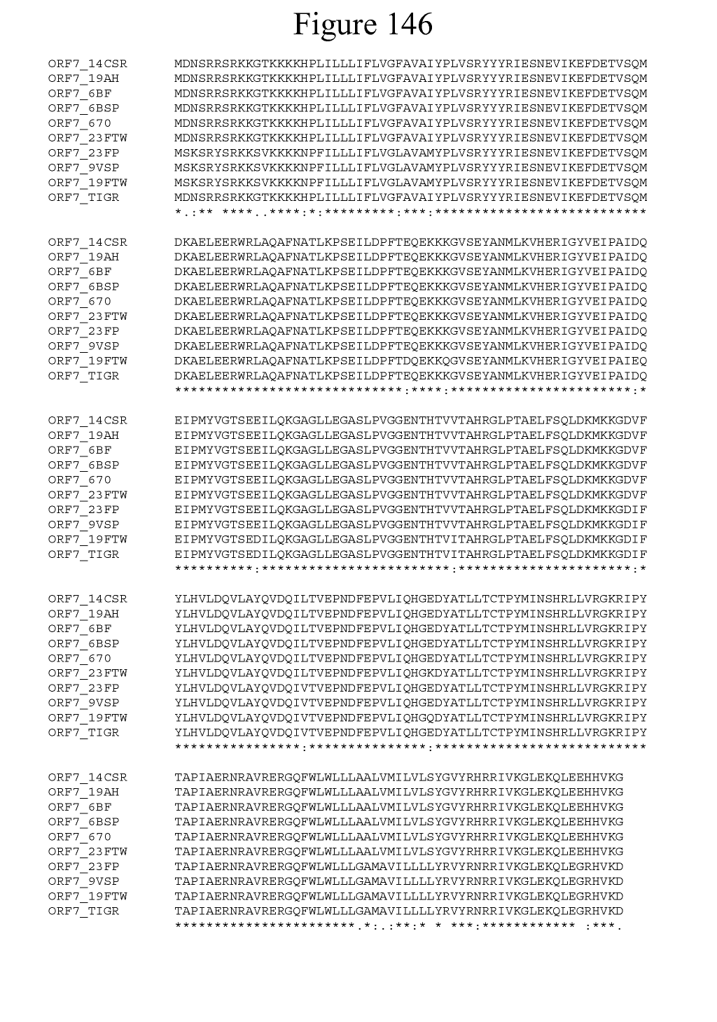

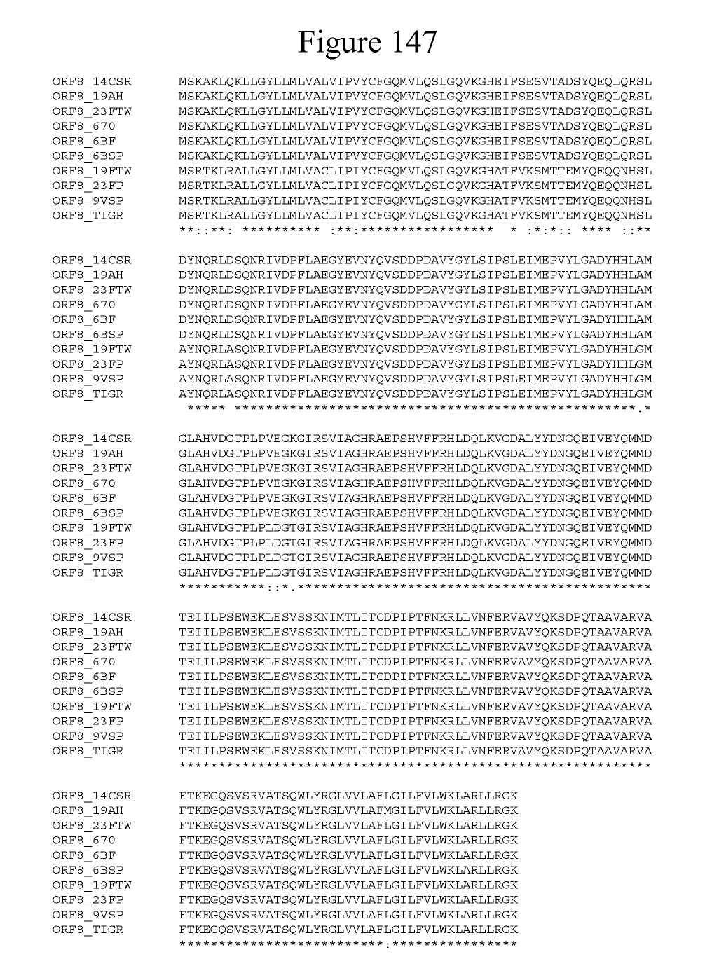

FIGS. 141-147 each provide a multiple sequence alignment for the polypeptides encoded by one of the open reading frames in all ten AI-positive S. pneumoniae strains. In each of the sequence alignments, light shading indicates an LPXTG motif and dark shading indicates the presence of an E-box motif with the conserved glutamic acid residue of the E-box motif in bold.

The sequence alignments also revealed that the polypeptides encoded by most of the open reading frames may be divided into two groups of homology, S. pneumoniae AI-a and AI-b. S. pneumoniae strains that comprise AI-a include 14 CSR 10, 19A Hungary 6, 23F Poland 15, 670, 6B Finland 12, and 6B Spain 2. S. pneumoniae strains that comprise AI-b include 19F Taiwan 14, 9V Spain 3, 23F Taiwan 15, and TIGR4. An immunogenic composition of the invention may comprise one or more polypeptides from within each of S. pneumoniae AI-a and AI-b. For example, polypeptide RrgB, encoded by open reading frame 4, may be divided within two such groups of homology. One group contains the RrgB sequences of six S. pneumoniae strains and a second group contains the RrgB sequences of four S. pneumoniae strains. While the amino acid sequence of the strains within each individual group is 99-100 percent identical, the amino acid sequence identity of the strains in the first relative to the second group is only 48%. Table 41 provides the identity comparisons of the amino acid sequences encoded by each open reading frame for the ten S. pneumoniae strains.

TABLE 42
Conservation of amino acid sequences encoded
by the S. pneumoniae AI locus

| | | | | % Identity |
|---|---|---|---|---|
| Putative Role | Encoded | Groups of | % Identity | Between |
| of Polypeptide | by Orf | Homology | in Group | Groups |
| RlrA, transcrip- | 2 | 1 group (10 | 100 | — |
| tional regulator | | strains) | | |
| RrgA, cell wall | 3 | 2 groups (6 + 4) | 98-100 | 83 |
| surface protein | | | | |
| RrgB, cell wall | 4 | 2 groups (6 + 4) | 99-100 | 48 |
| surface protein | | | | |
| RrgC, cell wall | 5 | 2 groups (6 + 4) | 99-100 | 97 |
| surface protein | | | | |
| SrtB, putative | 6 | 2 groups (7 + 3) | 99-100 | 97 |
| sortase | | | | |
| SrtC, putative | 7 | 2 groups (6 + 4) | 95-100 | 93 |
| sortase | | | | |
| SrtD, putative | 8 | 2 groups (6 + 4) | 99-100 | 92 |
| sortase | | | | |

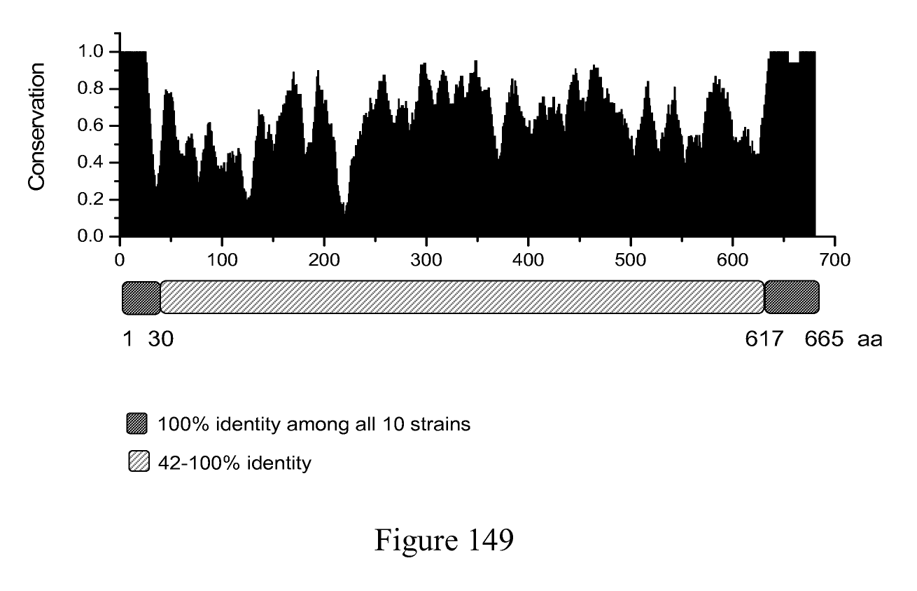

The division of homology between the RrgB polypeptide in the S. pneumoniae strains is due a lack of amino acid sequence identity in the central amino acid residues. Amino acid residues 1-30 and 617-665 are identical for each of the ten S. pneumoniae strains. However, amino acid residues 31-616 share between 42 and 100 percent identity between strains. See FIG. 149. The shared N- and C-terminal regions of identity in the RrgB polypeptides may be preferred portions of the RrgB polypeptide for use in an immunogenic composition. Similarly, shared regions of identity in any of the polypeptides encoded by the S. pneumoniae AI locus may be preferable for use in immunogenic compositions. One of skill in the art, using the amino acid alignments provided in FIGS. 141-147, would readily be able to determine these regions of identity.

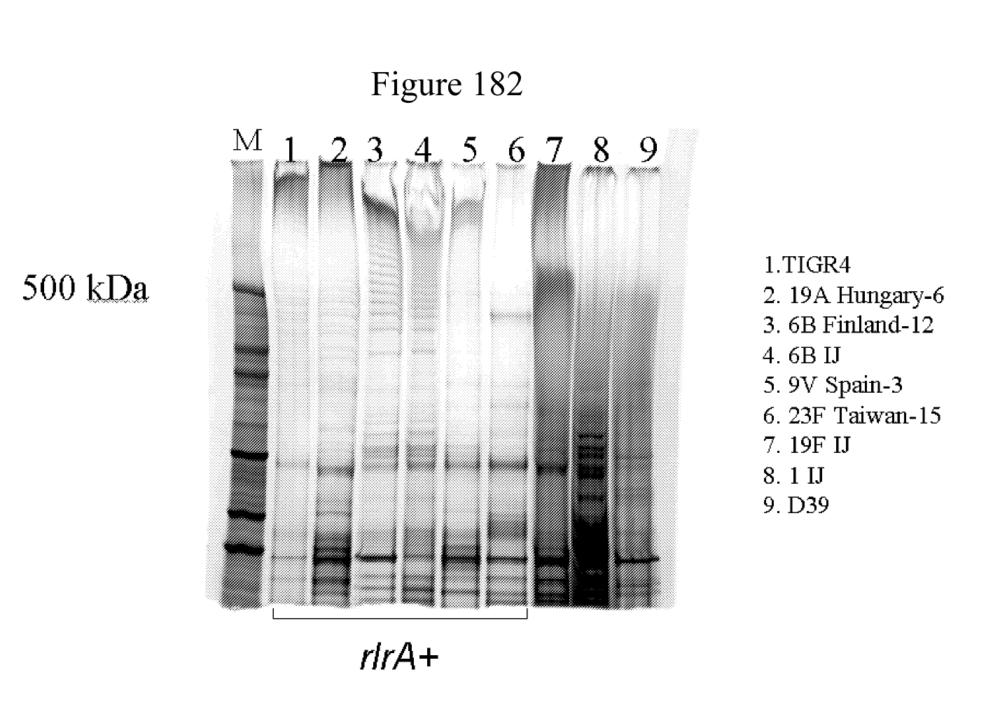

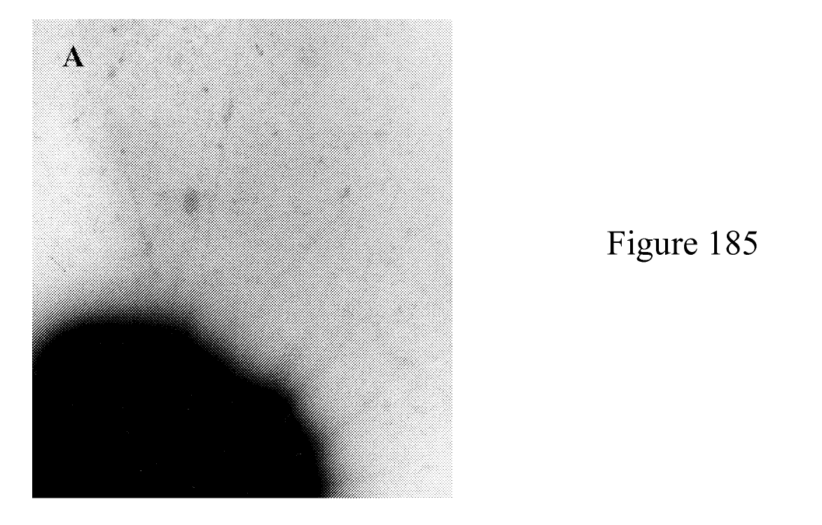

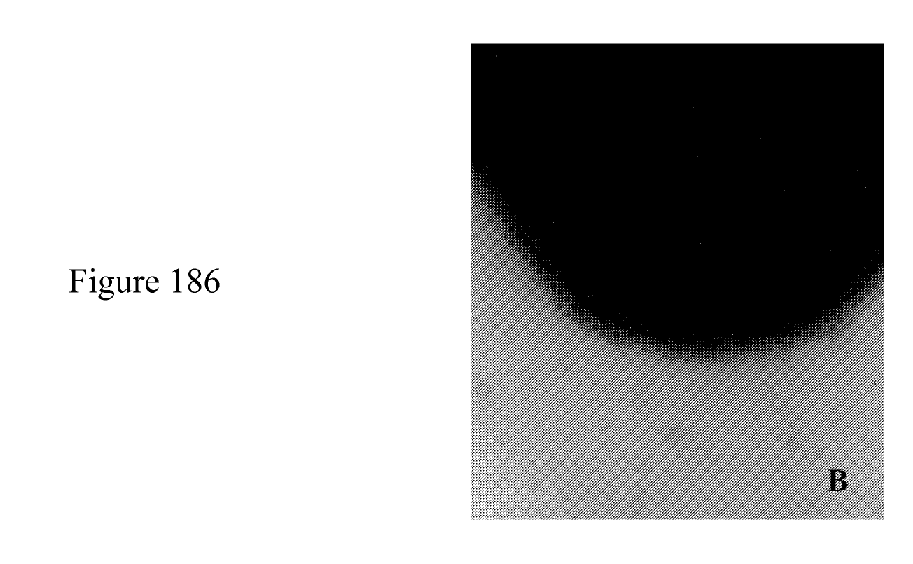

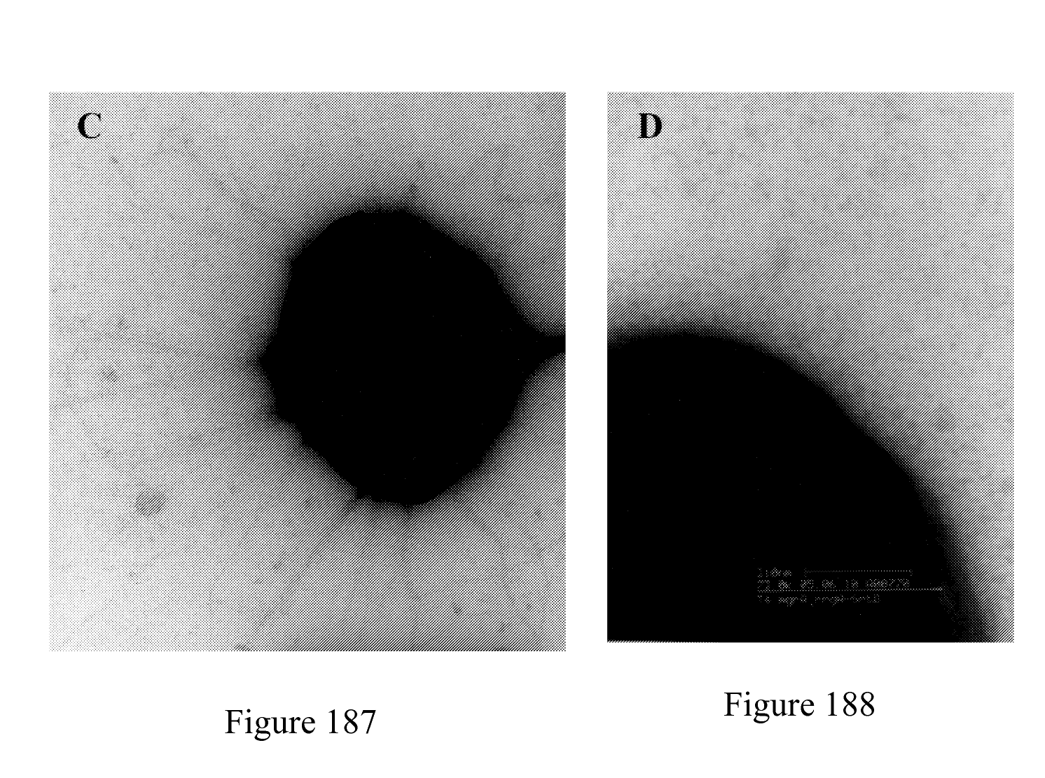

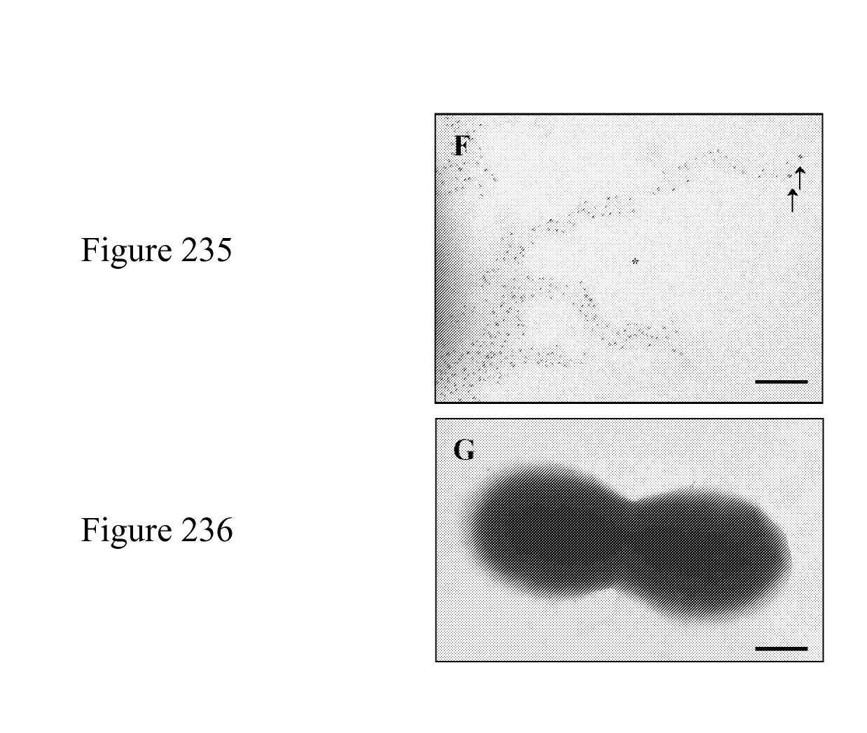

The S. pneumoniae comprising these AI loci do, in fact, express high molecular weight polymers on their surface, indicating the presence of pili. See FIG. 182, which shows detection of high molecular weight structures expressed by S. pneumoniae strains that comprise the adhesin island locus depicted in FIG. 137, these strains are indicated as rlrA+. Confirming these findings, electron microscopy and negative staining detects the presence of pili extending from the surface of S. pneumoniae. See FIG. 185. To demonstrate that the adhesin island locus was responsible for the pili, the rrgA-srtD region of TIGR 4 were deleted. Deletion of this region of the adhesin island resulted in a loss of pili expression. See FIG. 186. See also FIG. 235, which provides an electron micrograph of S. pneumoniae lacking the rrgA-srtD region immunogold stained using anti-RrgB and anti-RrgC antibodies. No pili can be seen. Similarly to that described above, a S. pneumoniae bacteria that lacks a transcriptional repressor, mgrA, of genes in the adhesin island expresses pili. See FIG. 187. However, and as expected, a S. pneumoniae bacteria that lacks both the mgrA and adhesin island genes in the rrgA-srtD region does not express pili. See FIG. 188.

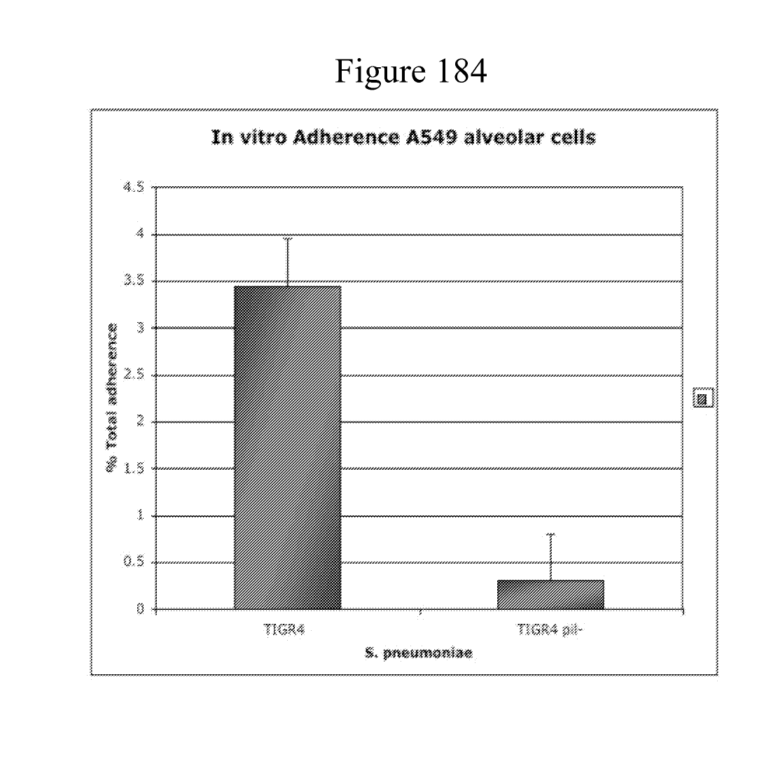

These high molecular weight pili structures appear to play a role in adherence of S. pneumoniae to cells. S. pneumoniae TIGR4 that lack the pilus operon have significantly diminished ability to adhere to A549 alveolar cells in vitro. See FIG. 184.

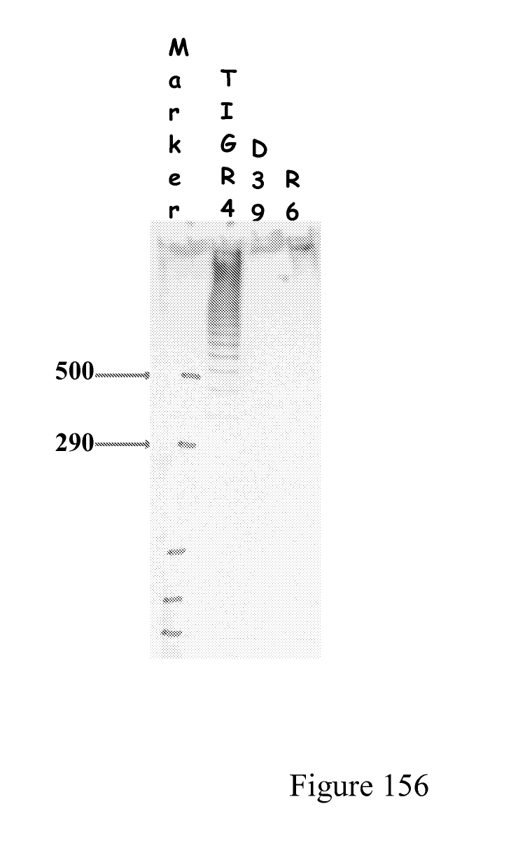

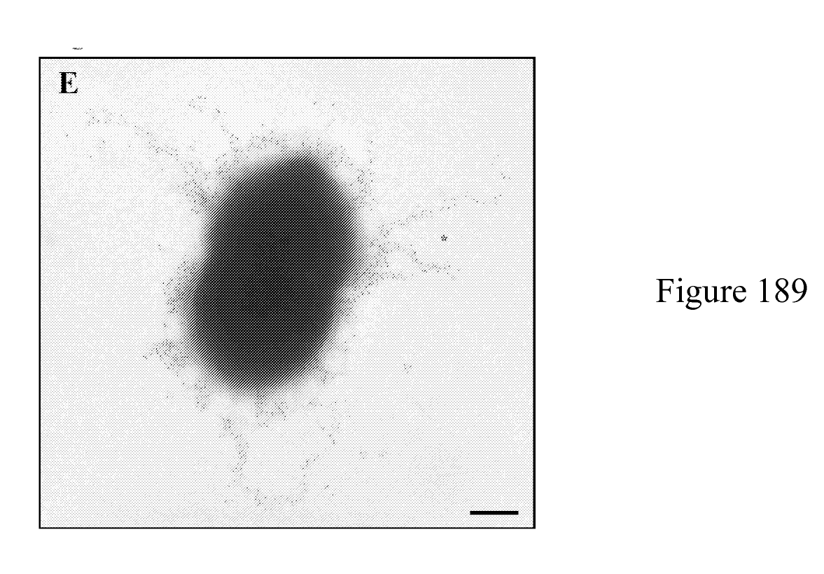

The Sp0463 (S. pneumoniae TIGR4 rrgB) adhesion island polypeptide is expressed in oligomeric form. Whole cell extracts were analyzed by Western blot using a Sp0463 antiserum. The antiserum cross-hybridized with high molecular weight Sp0463 polymers. See FIG. 156. The antiserum did not cross-hybridize with polypeptides from D39 or R6 strains of S. pneumoniae, which do not contain the AI locus depicted in FIG. 137. Immunogold labelling of S. pneumoniae TIGR 4 using RrgB antiserum confirms the presence of RrgB in pili. FIG. 189 shows double-labeling of S. pneumoniae TIGR 4 bacteria with immunolabeling for RrgB (5 nm gold particles) and RrgC (10 nm gold particles) protein. The RrgB protein is detected as present at intervals along the pilus structure. The RrgC protein is detected at the tips of the pili. See FIG. 234 at arrows; FIG. 234 is a close up of a pilus in FIG. 189 at the location indicated by *.

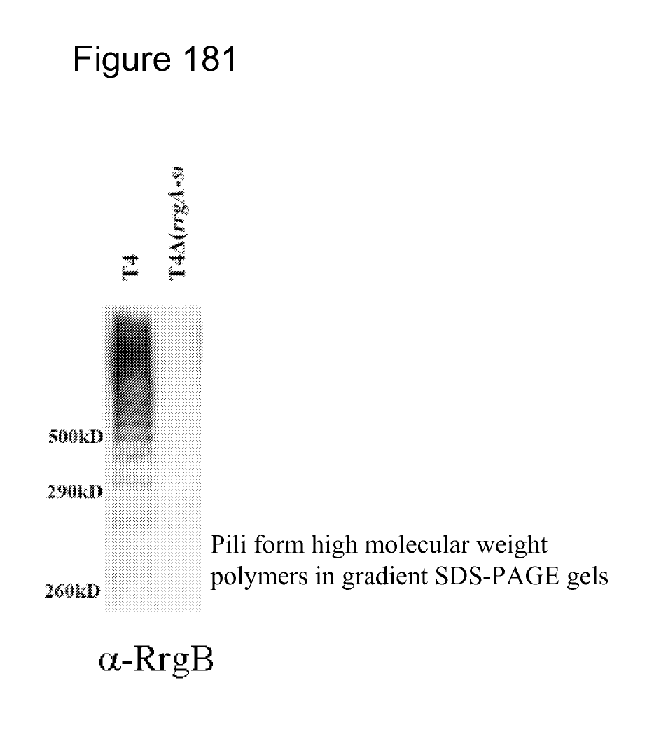

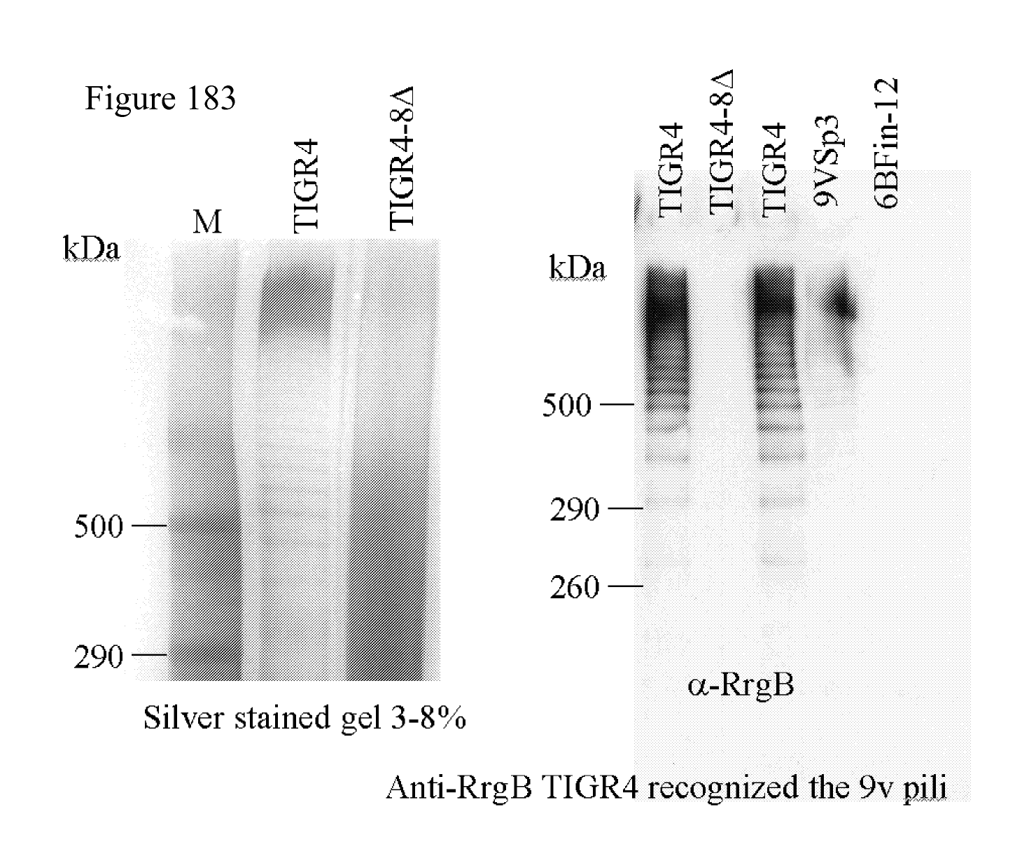

The RrgA protein appears to be present in and necessary for formation of high molecular weight structures on the surface of S. pneumoniae TIGR4. See FIG. 181 which provides the results of Western blot analysis of TIGR4 S. pneumoniae lacking the gene encoding RrgA. No high molecular weight structures are detected in S. pneumoniae that do not express RrgA using antiserum raised against RrgB. See also FIG. 183.

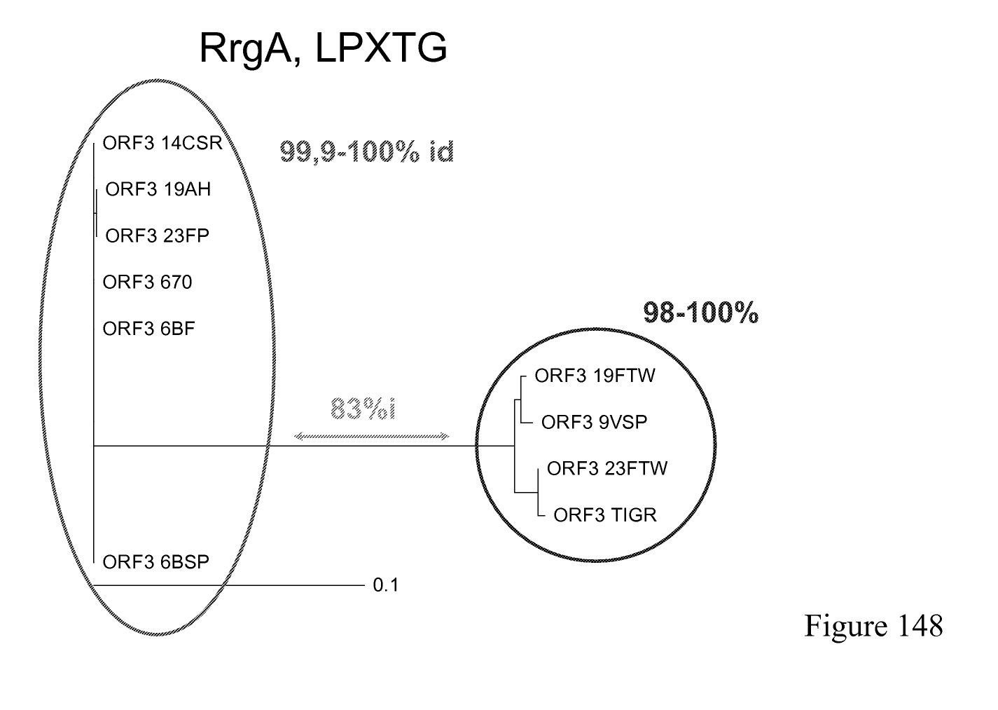

A detailed diagram of the amino acid sequence comparisons of the RrgA protein in the ten S. pneumoniae strains is shown in FIG. 148. The diagram reveals the division of the individual S. pneumoniae strains into the two different homology groups.

The cell surface polypeptides encoded by the S. pneumoniae TIGR4 AI, Sp0462 (rrgA), Sp0463 (rrgB), and Sp0464 (rrgC), have been cloned and expressed. See examples 15-17. A polyacrylamide gel showing successful recombinant expression of RrgA is provided in FIG. 190A. Detection of the RrgA protein, which is expressed in pET21b with a histidine tag, is also shown by Western blot analysis in FIG. 190B, using an anti-histidine tag antibody.

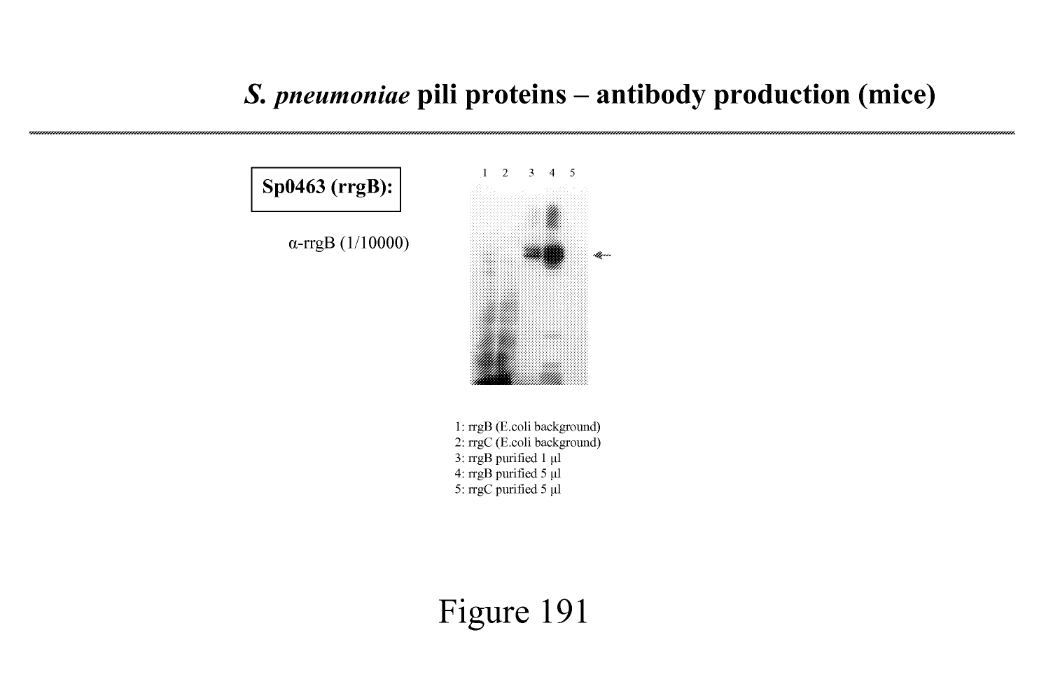

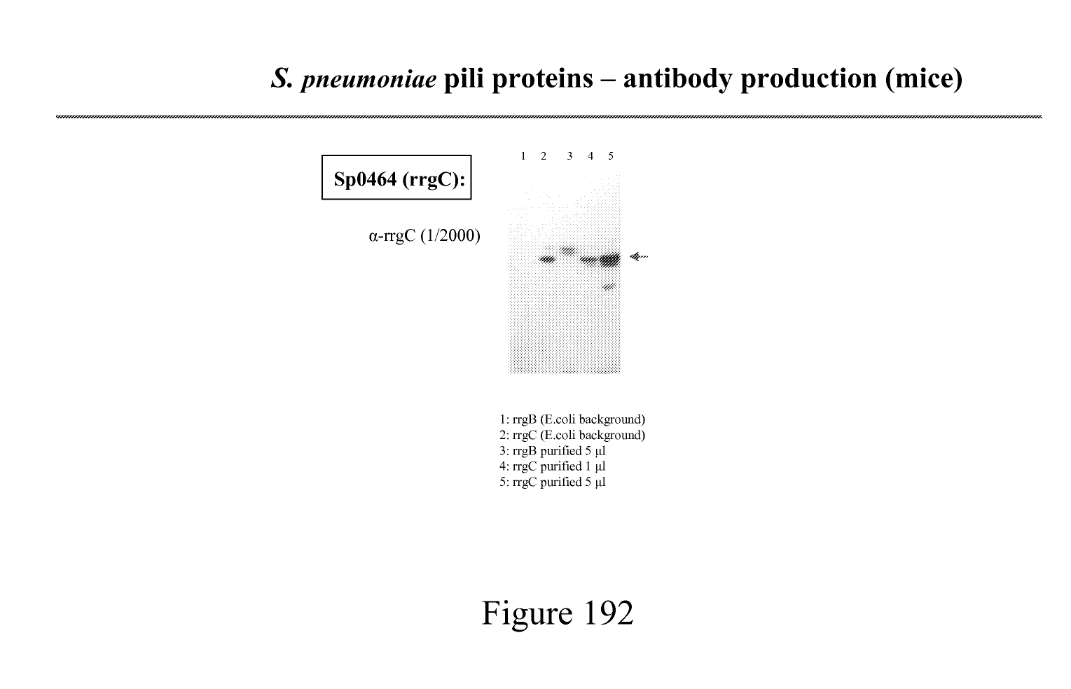

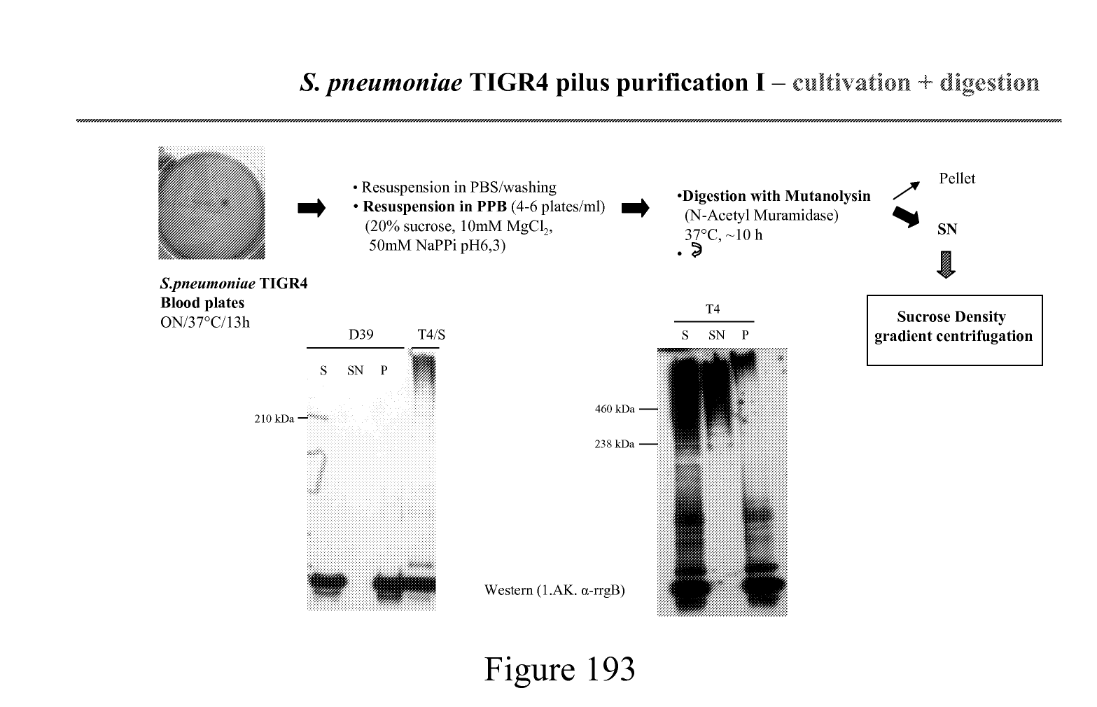

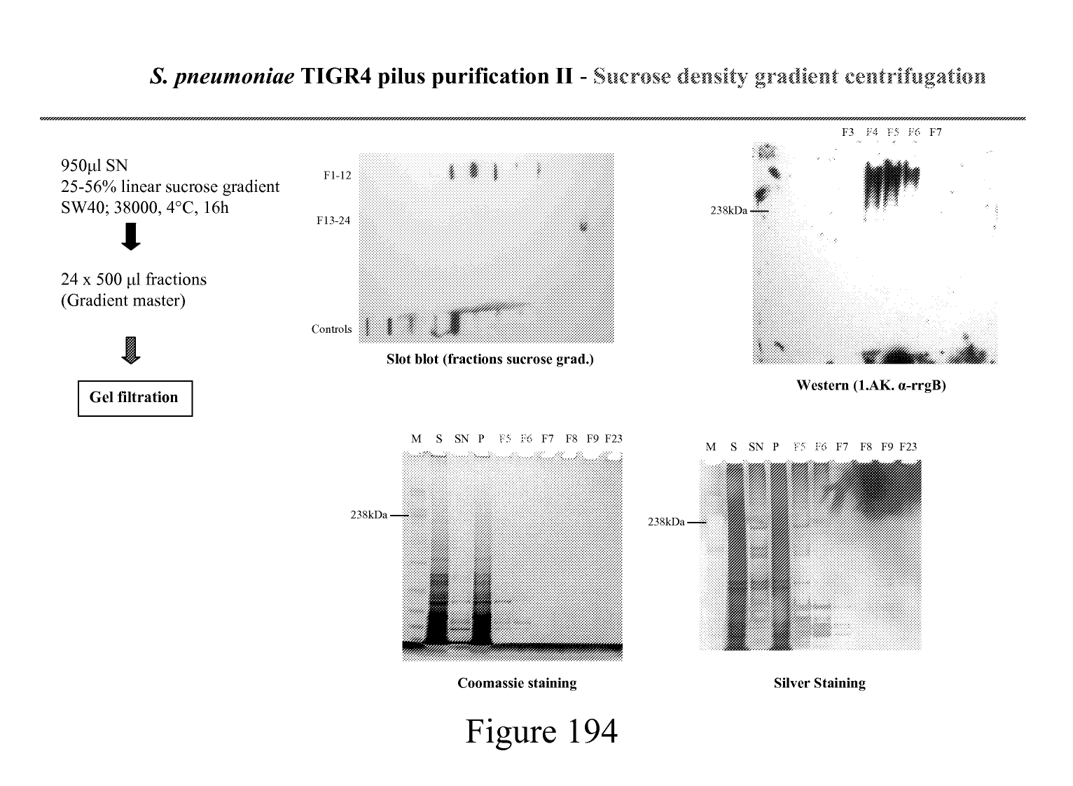

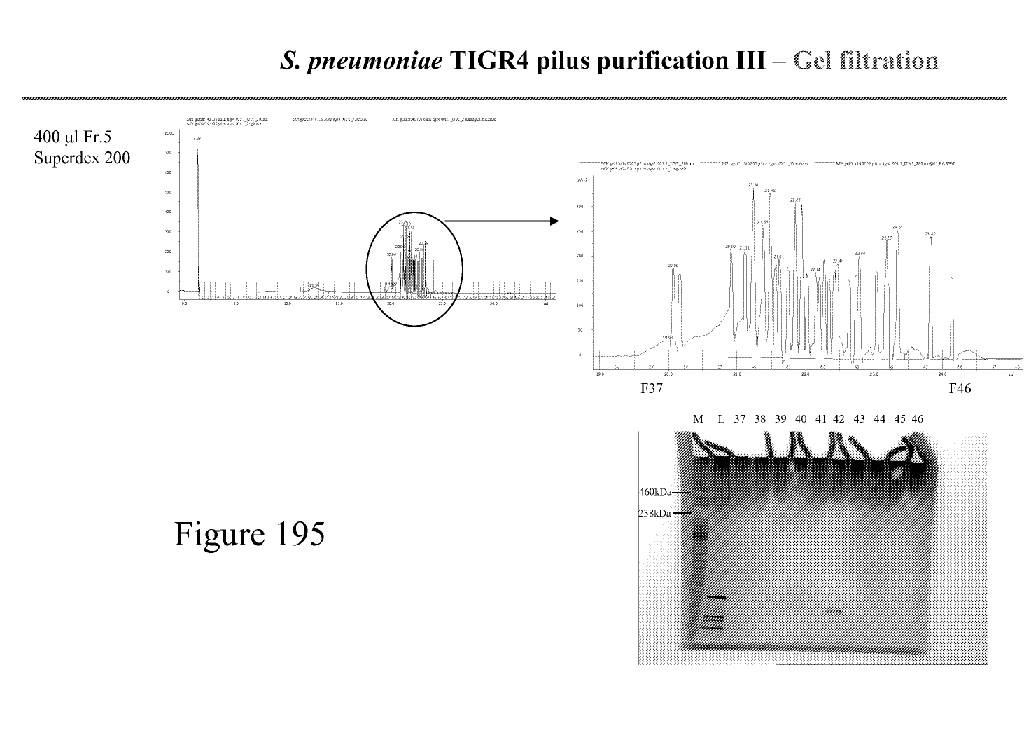

Antibodies that detect RrgB and RrgC antibodies have been produced in mice. See FIGS. 191 and 192, which show detection of RrgB and RrgC, respectively, using the raised antibodies.

In addition to the identification of these S. pneumoniae adhesion islands, coding sequences for SrtB type sortases have been identified in several S. pneumoniae clinical isolates, demonstrating conservation of a SrtB type sortase across these isolates.

### Recombinantly Produced AI Polypeptides

It is also an aspect of the invention to alter a non-AI polypeptide to be expressed as an AI polypeptide. The non-AI polypeptide may be genetically manipulated to additionally contain AI polypeptide sequences, e.g., a sortase substrate, pilin, or E-box motif, which may cause expression of the non-AI polypeptide as an AI polypeptide. Alternatively the non-AI polypeptide may be genetically manipulated to replace an amino acid sequence within the non-AI polypeptide for AI polypeptide sequences, e.g., a sortase substrate, pilin, or E-box motif, which may cause expression of the non-AI polypeptide as an AI polypeptide. Any number of amino acid residues may be added to the non-AI polypeptide or may be replaced within the non-AI polypeptide to cause its expression as an AI polypeptide. At least 5, 6, 7, 8, 9, 10, 15, 20, 25, 30, 35, 50, 75, 100, 150, 200, or 250 amino acid residues may be replaced or added to the non-AI polypeptide amino acid sequence. GBS 322 may be one such non-AI polypeptide that may be expressed as an AI polypeptide.

### GBS Adhesin Island Sequences

The GBS AI polypeptides of the invention can, of course, be prepared by various means (e.g. recombinant expression, purification from GBS, chemical synthesis etc.) and in various forms (e.g. native, fusions, glycosylated, non-glycosylated etc.). They are preferably prepared in substantially pure form (i.e. substantially free from other streptococcal or host cell proteins) or substantially isolated form.

The GBS AI proteins of the invention may include polypeptide sequences having sequence identity to the identified GBS proteins. The degree of sequence identity may vary depending on the amino acid sequence (a) in question, but is preferably greater than 50% (e.g. 60%, 65%, 70%, 75%, 80%, 85%, 90%, 91%, 92%, 93%, 94%, 95%, 96%, 97%, 98%, 99%, 99.5% or more). Polypeptides having sequence identity include homologs, orthologs, allelic variants and functional mutants of the identified GBS proteins. Typically, 50% identity or more between two proteins is considered to be an indication of functional equivalence. Identity between proteins is preferably determined by the Smith-Waterman homology search algorithm as implemented in the MPSRCH program (Oxford Molecular), using an affinity gap search with parameters gap open penalty=12 and gap extension penalty=1.

The GBS adhesin island polynucleotide sequences may include polynucleotide sequences having sequence identity to the identified GBS adhesin island polynucleotide sequences. The degree of sequence identity may vary depending on the polynucleotide sequence in question, but is preferably greater than 50% (e.g. 60%, 65%, 70%, 75%, 80%, 85%, 90%, 91%, 92%, 93%, 94%, 95%, 96%, 97%, 98%, 99%, 99.5% or more).

The GBS adhesin island polynucleotide sequences of the invention may include polynucleotide fragments of the identified adhesin island sequences. The length of the fragment may vary depending on the polynucleotide sequence of the specific adhesin island sequence, but the fragment is preferably at least 10 consecutive polynucleotides, (e.g. at least 10, 12, 14, 16, 18, 20, 25, 30, 35, 40, 50, 60, 70, 80, 90, 100, 150, 200 or more).

The GBS adhesin island amino acid sequences of the invention may include polypeptide fragments of the identified GBS proteins. The length of the fragment may vary depending on the amino acid sequence of the specific GBS antigen, but the fragment is preferably at least 7 consecutive amino acids, (e.g. 8, 10, 12, 14, 16, 18, 20, 25, 30, 35, 40, 50, 60, 70, 80, 90, 100, 150, 200 or more). Preferably the fragment comprises one or more epitopes from the sequence. Other preferred fragments include (1) the N-terminal signal peptides of each identified GBS protein, (2) the identified GBS protein without their N-terminal signal peptides, and (3) each identified GBS protein wherein up to 10 amino acid residues (e.g. 1, 2, 3, 4, 5, 6, 7, 8, 9, 10, 15, 20, 25 or more) are deleted from the N-terminus and/or the C-terminus e.g. the N-terminal amino acid residue may be deleted. Other fragments omit one or more domains of the protein (e.g. omission of a signal peptide, of a cytoplasmic domain, of a transmembrane domain, or of an extracellular domain).

### GBS 80

Examples of preferred GBS 80 fragments are discussed below. Polynucleotide and polypeptide sequences of GBS 80 from a variety of GBS serotypes and strain isolates are set forth in FIGS. 18 and 22. The polynucleotide and polypeptide sequences for GBS 80 from GBS serotype V, strain isolate 2603 are also included below as SEQ ID NOS 1 and 2:

SEQ ID NO. 1

ATGAAATTATCGAAGAAGTTATTGTTTTCGGCTGCTGTTTTAACAATGGT
GGCGGGGTCAACTGTTGAACCAGTAGCTCAGTTTGCGACTGGAATGAGTA
TTGTAAGAGCTGCAGAAGTGTCACAAGAACGCCCAGCGAAAACAACAGTA
AATATCTATAAATTACAAGCTGATAGTTATAAATCGGAAATTACTTCTAA
TGGTGGTATCGAGAATAAAGACGGCGAAGTAATATCTAACTATGCTAAAC
TTGGTGACAATGTAAAAGGTTTGCAAGGTGTACAGTTTAAACGTTATAAA
GTCAAGACGGATATTTCTGTTGATGAATTGAAAAAATTGACAACAGTTGA
AGCAGCAGATGCAAAAGTTGGAACGATTCTTGAAGAAGGTGTCAGTCTAC
CTCAAAAAACTAATGCTCAAGGTTTGGTCGTCGATGCTCTGGATTCAAAA
AGTAATGTGAGATACTTGTATGTAGAAGATTTAAAGAATTCACCTTCAAA
CATTACCAAAGCTTATGCTGTACCGTTTGTGTTGGAATTACCAGTTGCTA
ACTCTACAGGTACAGGTTTCCTTTCTGAAATTAATATTTACCCTAAAAAC
GTTGTAACTGATGAACCAAAAACAGATAAAGATGTTAAAAAATTAGGTCA
GGACGATGCAGGTTATACGATTGGTGAAGAATTCAAATGGTTCTTGAAAT
CTACAATCCCTGCCAATTTAGGTGACTATGAAAAATTTGAAATTACTGAT
AAATTTGCAGATGGCTTGACTTATAAATCTGTTGGAAAAATCAAGATTGG
TTCGAAAACACTGAATAGAGATGAGCACTACACTATTGATGAACCAACAG
TTGATAACCAAAATACATTAAAAATTACGTTTAAACCAGAGAAATTTAAA
GAAATTGCTGAGCTACTTAAAGGAATGACCCTTGTTAAAAATCAAGATGC
TCTTGATAAAGCTACTGCAAATACAGATGATGCGGCATTTTTGGAAATTC
CAGTTGCATCAACTATTAATGAAAAAGCAGTTTTAGGAAAAGCAATTGAA
AATACTTTTGAACTTCAATATGACCATACTCCTGATAAAGCTGACAATCC
AAAACCATCTAATCCTCCAAGAAAACCAGAAGTTCATACTGGTGGGAAAC
GATTTGTAAAGAAAGACTCAACAGAAACACAAACACTAGGTGGTGCTGAG
TTTGATTTGTTGGCTTCTGATGGGACAGCAGTAAAATGGACAGATGCTCT
TATTAAAGCGAATACTAATAAAAACTATATTGCTGGAGAAGCTGTTACTG
GGCAACCAATCAAATTGAAATCACATACAGACGGTACGTTTGAGATTAAA
GGTTTGGCTTATGCAGTTGATGCGAATGCAGAGGGTACAGCAGTAACTTA
CAAATTAAAAGAAACAAAAGCACCAGAAGGTTATGTAATCCCTGATAAAG
AAATCGAGTTTACAGTATCACAAACATCTTATAATACAAAACCAACTGAC
ATCACGGTTGATAGTGCTGATGCAACACCTGATACAATTAAAAACAACAA
ACGTCCTTCAATCCCTAATACTGGTGGTATTGGTACGGCTATCTTTGTCG
CTATCGGTGCTGCGGTGATGGCTTTTGCTGTTAAGGGGATGAAGCGTCGT
ACAAAAGATAAC

SEQ ID NO: 2

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNNKRPSIPNTGGIGTAIFVAIGAAVMAFAVKGMKRR
TKDN

As described above, the compositions of the invention may include fragments of AI proteins. In some instances, removal of one or more domains, such as a leader or signal sequence region, a transmembrane region, a cytoplasmic region or a cell wall anchoring motif, may facilitate cloning of the gene encoding the protein and/or recombinant expression of the GBS AI protein. In addition, fragments comprising immunogenic epitopes of the cited GBS AI proteins may be used in the compositions of the invention.

For example, GBS 80 contains an N-terminal leader or signal sequence region which is indicated by the underlined sequence at the beginning of SEQ ID NO:2 above. In one embodiment, one or more amino acids from the leader or signal sequence region of GBS 80 are removed. An example of such a GBS 80 fragment is set forth below as SEQ ID NO:3:

SEQ ID NO: 3

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKGMTLVKNQDALDKATANTDDAAFLEIPVAS
TINEKAVLGKAIENTFELQYDHTPDKADNPKPSNPPRKPEVHTGGKRFVK
KDSTETQTLGGAEFDLLASDGTAVKWTDALIKANTNKNYIAGEAVTGQPI
KLKSHTDGTFEIKGLAYAVDANAEGTAVTYKLKETKAPEGYVIPDKEIEF
TVSQTSYNTKPTDITVDSADATPDTIKNKKRPSIPNTGGIGTAIFVAIGA
AVMAFAVKGMKRRTKDN

GBS 80 contains a C-terminal transmembrane region which is indicated by the underlined sequence near the end of SEQ ID NO:2 above. In one embodiment, one or more amino acids from the transmembrane region and/or a cytoplasmic region are removed. An example of such a GBS 80 fragment is set forth below as SEQ ID NO:4:

SEQ ID NO: 4

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNKKRPSIPNTG

GBS 80 contains an amino acid motif indicative of a cell wall anchor: SEQ ID NO:5 IPNTG (shown in italics in SEQ ID NO:2 above). In some recombinant host cell systems, it may be preferable to remove this motif to facilitate secretion of a recombinant GBS 80 protein from the host cell. Accordingly, in one preferred fragment of GBS 80 for use in the invention, the transmembrane and/or cytoplasmic regions and the cell wall anchor motif are removed from GBS 80. An example of such a GBS 80 fragment is set forth below as SEQ ID NO:6.

SEQ ID NO: 6

MKLSKKLLFSAAVLTMVAGSTVEPVAQFATGMSIVPAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDITKNKKRPS

Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

In one embodiment, the leader or signal sequence region, the transmembrane and cytoplasmic regions and the cell wall anchor motif are removed from the GBS 80 sequence. An example of such a GBS 80 fragment is set forth below as SEQ ID NO:7.

SEQ ID NO: 7

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKGMTLVKNQDALDKATANTDDAAFLEIPVAS
TINEKAVLGKATENTFELQYDHTPDKADNPKPSNPPRKPEVHTGGKRFVK
KDSTETQTLGGAEFDLLASDGTAVKWTDALIKANTNKNYIAGEAVTGQPI
KLKSHTDGTFEIKGLAYAVDANAEGTAVTYKLKETKAPEGYVIPDKEIEF
TVSQTSYNTKPTDITVDSADATPDTIKNKKRPS

Applicants have identified a particularly immunogenic fragment of the GBS 80 protein. This immunogenic fragment is located towards the N-terminus of the protein and is underlined in the GBS 80 SEQ ID NO:2 sequence below. The underlined fragment is set forth below as SEQ ID NO:8.

SEQ ID NO: 2

MKLSKKLLFSAAVLTMVAGSTVEPVAQFATGMSIVPAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPOKLKSHTDGTFEIK
GLAYAVDANAEGTAVTYKLKETKAPEGYVIPDKEIEFTVSQTSYNTKPTD
ITVDSADATPDTIKNNKRPSIPNTGGIGTAIFVAIGAAVMAFAVKGMKRR
TKDN

SEQ ID NO: 8

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKG

The immunogenicity of the protein encoded by SEQ ID NO:7 was compared against PBS, GBS whole cell, GBS 80 (full length) and another fragment of GBS 80, located closer to the C-terminus of the peptide (SEQ ID NO:9, below).

SEQ ID NO: 9

MTLVKNQDALDKATANTDDAAFLEIPVASTINEKAVLGKAIENTFELQYD
HTPDKADNPKPSNPPRKPEVHTGGKRFVKKDSTETQTLGGAEFDLLASDG
TAVKWTDALIKANTNKNYIAGEAVTGQPIKLKSHTDGTFEIKGLAYAVDA
NAEGTAVTYKLKETKAPEGYVIPDKEIEFTVSQTSYNTKPTDITVDSADA
TPDTIKNNKRPS

Both an Active Maternal Immunization Assay and a Passive Maternal Immunization Assay were conducted on this collection of proteins.

As used herein, an Active Maternal Immunization assay refers to an in vivo protection assay where female mice are immunized with the test antigen composition. The female mice are then bred and their pups are challenged with a lethal dose of GBS. Serum titers of the female mice during the immunization schedule are measured as well as the survival time of the pups after challenge.

Specifically, the Active Maternal Immunization assays referred to herein used groups of four CD-1 female mice (Charles River Laboratories, Calco Italy). These mice were immunized intraperitoneally with the selected proteins in Freund's adjuvant at days 1, 21 and 35, prior to breeding. 6-8 weeks old mice received 20 μg protein/dose when immunized with a single antigen, 30-45 μg protein/dose (15 μg each antigen) when immunized with combination of antigens. The immune response of the dams was monitored by using serum samples taken on day 0 and 49. The female mice were bred 2-7 days after the last immunization (at approximately t=36-37), and typically had a gestation period of 21 days. Within 48 hours of birth, the pups were challenged via I.P. with GBS in a dose approximately equal to a amount which would be sufficient to kill 70-90% of unimmunized pups (as determined by empirical data gathered from PBS control groups). The GBS challenge dose is preferably administered in 50 W of THB medium. Preferably, the pup challenge takes place at 56 to 61 days after the first immunization. The challenge inocula were prepared starting from frozen cultures diluted to the appropriate concentration with THB prior to use. Survival of pups was monitored for 5 days after challenge.

As used herein, the Passive Maternal Immunization Assay refers to an in vivo protection assay where pregnant mice are passively immunized by injecting rabbit immune sera (or control sera) approximately 2 days before delivery. The pups are then challenged with a lethal dose of GBS.

Specifically, the Passive Maternal Immunization Assay referred to herein used groups of pregnant CD1 mice which were passively immunized by injecting 1 ml of rabbit immune sera or control sera via I.P., 2 days before delivery. Newborn mice (24-48 hrs after birth) are challenged via I.P. with a 70-90% lethal dose of GBS serotype III COH1. The challenge dose, obtained by diluting a frozen mid log phase culture, was administered in 50 μl of THB medium.

For both assays, the number of pups surviving GBS infection was assessed every 12 hrs for 4 days. Statistical significance was estimated by Fisher's exact test.

The results of each assay for immunization with SEQ ID NO:7, SEQ ID NO:8, PBS and GBS whole cell are set forth in Tables 1 and 2 below.

TABLE 1
Immunization

| | Alive/ | % | Fisher's |
|---|---|---|---|
| Antigen | total | Survival | exact test |
| PBS (neg control) | 13/80 | 16% | |
| GBS (whole cell) | 54/65 | 83% | P < 0.00000001 |
| GBS80 (intact) | 62/70 | 88% | P < 0.00000001 |
| GBS80 (fragment) SEQ ID 7 | 35/64 | 55% | P = 0.0000013 |
| GBS80 (fragment) SEQ ID 8 | 13/67 | 19% | P = 0.66 |

TABLE 2
Passive Maternal Immunization

| | Alive/ | % | Fisher's |
|---|---|---|---|
| Antigen | total | Survival | exact test |
| PBS (neg control) | 12/42 | 28% | |
| GBS (whole cell) | 48/52 | 92% | P < 0.00000001 |
| GBS80 (intact) | 48/55 | 87% | P < 0.00000001 |
| GBS80 (fragment) SEQ ID 7 | 45/57 | 79% | P = 0.0000006 |
| GBS80 (fragment) SEQ ID 8 | 13/54 | 24% | P = 1 |

As shown in Tables 1 and 2, immunization with the SEQ ID NO:7 GBS 80 fragment provided a substantially improved survival rate for the challenged pups than the comparison SEQ ID NO:8 GBS 80 fragment. These results indicate that the SEQ ID NO:7 GBS 80 fragment may comprise an important immunogenic epitope of GBS 80.

As discussed above, pilin motifs, containing conserved lysine (K) residues have been identified in GBS 80. The pilin motif sequences are underlined in SEQ ID NO:2, below. Conserved lysine (K) residues are marked in bold, at amino acid residues 199 and 207 and at amino acid residues 368 and 375. The pilin sequences, in particular the conserved lysine residues, are thought to be important for the formation of oligomeric, pilus-like structures of GBS 80. Preferred fragments of GBS 80 include at least one conserved lysine residue. Preferably, fragments include at least one pilin sequence.

SEQ ID NO: 2

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKLNRDEHYTIDEPTVDNQNTLKITFKPEKFKE
IAELLKGMTLVKNQDALDKATANTDDAAFLEIPVASTINEKAVLGKAIEN
TFELQYDHTPDKADNPKPSNPPRKPEVHTGGKRFVKKDSTETQTLGGAEF
DLLASDGTAVKWTDALIKANTNKNYIAGEAVTGQPIKLKSHTDGTFEIKG
LAYAVDANAEGTAVTYKLKETKAPEGYVIPDKEIEFTVSQTSYNTKPTDI
TVDSADATPDTIKNNKRPSIPNTGGIGTAIFVAIGAAVMAFAVKGMKRRT
KDN

E boxes containing conserved glutamic residues have also been identified in GBS 80. The E box motifs are underlined in SEQ ID NO:2 below. The conserved glutamic acid (E) residues, at amino acid residues 392 and 471, are marked in bold. The E box motifs, in particular the conserved glutamic acid residues, are thought to be important for the formation of oligomeric pilus-like structures of GBS 80. Preferred fragments of GBS 80 include at least one conserved glutamic acid residue. Preferably, fragments include at least one E box motif.

SEQ ID NO: 2

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKLNRDEHYTIDEPTVDNQNTLKITFKPEKFKE
IAELLKGMTLVKNQDALDKATANTDDAAFLEIPVASTINEKAVLGKAIEN
TFELQYDHTPDKADNPKPSNPPRKPEVHTGGKRFVKKDSTETQTLGGAEF
DLLASDGTAVKWTDALIKANTNKNYIAGEAVTGQPIKLKSHTDGTFEIKG
LAYAVDANAEGTAVTYKLKETKAPEGYVIPDKEIEFTVSQTSYNTKPTDI
TVDSADATPDTIKNNKRPSIPNTGGIGTAIFVAIGAAVMAFAVKGMKRRT
KDN

### GBS 104

Similarly, the following offers examples of preferred GBS 104 fragments. Nucleotide and amino acid sequences of GBS 104 sequenced from serotype V isolated strain 2603 are set forth below as SEQ ID NOS 10 and 11:

SEQ ID NO. 10

ATGAAAAAGAGACAAAAAATATGGAGAGGGTTATCAGTTACTTTACTAAT
CCTGTCCCAAATTCCATTTGGTATATTGGTACAAGGTGAAACCCAAGATA
CCAATCAAGCACTTGGAAAAGTAATTGTTAAAAAAACGGGAGACAATGCT
ACACCATTAGGCAAAGCGACTTTTGTGTTAAAAAATGACAATGATAAGTC
AGAAACAAGTCACGAAACGGTAGAGGGTTCTGGAGAAGCAACCTTTGAAA
ACATAAAACCTGGAGACTACACATTAAGAGAAGAAACAGCACCAATTGGT
TATAAAAAAACTGATAAAACCTGGAAAGTTAAAGTTGCAGATAACGGAGC
AACAATAATCGAGGGTATGGATGCAGATAAAGCAGAGAAACGAAAAGAAG
TTTTGAATGCCCAATATCCAAAATCAGCTATTTATGAGGATACAAAAGAA
AATTACCCATTAGTTAATGTAGAGGGTTCCAAAGTTGGTGAACAATACAA
AGCATTGAATCCAATAAATGGAAAAGATGGTCGAAGAGAGATTGCTGAAG
GTTGGTTATCAAAAAAAATTACAGGGGTCAATGATCTCGATAAGAATAAA
TATAAAATTGAATTAACTGTTGAGGGTAAAACCACTGTTGAAACGAAAGA
ACTTAATCAACCACTAGATGTCGTTGTGCTATTAGATAATTCAAATAGTA
TGAATAATGAAAGAGCCAATAATTCTCAAAGAGCATTAAAAGCTGGGGAA
GCAGTTGAAAAGCTGATTGATAAAATTACATCAAATAAAGACAATAGAGT
AGCTCTTGTGACATATGCCTCAACCATTTTTGATGGTACTGAAGCGACCG
TATCAAAGGGAGTTGCCGATCAAAATGGTAAAGCGCTGAATGATAGTGTA
TCATGGGATTATCATAAAACTACTTTTACAGCAACTACACATAATTACAG
TTATTTAAATTTAACAAATGATGCTAACGAAGTTAATATTCTAAAGTCAA
GAATTCCAAAGGAAGCGGAGCATATAAATGGGGATCGCACGCTCTATCAA
TTTGGTGCGACATTTACTCAAAAAGCTCTAATGAAAGCAAATGAAATTTT
AGAGACACAAAGTTCTAATGCTAGAAAAAAACTTATTTTTCACGTAACTG
ATGGTGTCCCTACGATGTCTTATGCCATAAATTTTAATCCTTATATATCA
ACATCTTACCAAAACCAGTTTAATTCTTTTTTAAATAAAATACCAGATAG
AAGTGGTATTCTCCAAGAGGATTTTATAATCAATGGTGATGATTATCAAA
TAGTAAAAGGAGATGGAGAGAGTTTTAAACTGTTTTCGGATAGAAAAGTT
CCTGTTACTGGAGGAACGACACAAGCAGCTTATCGAGTACCGCAAAATCA
ACTCTCTGTAATGAGTAATGAGGGATATGCAATTAATAGTGGATATATTT
ATCTCTATTGGAGAGATTACAACTGGGTCTATCCATTTGATCCTAAGACA
AAGAAAGTTTCTGCAACGAAACAAATCAAAACTCATGGTGAGCCAACAAC
ATTATACTTTAATGGAAATATAAGACCTAAAGGTTATGACATTTTTACTG
TTGGGATTGGTGTAAACGGAGATCCTGGTGCAACTCCTCTTGAAGCTGAG
AAATTTATGCAATCAATATCAAGTAAAACAGAAAATTATACTAATGTTGA
TGATACAAATAAAATTTATGATGAGCTAAATAAATACTTTAAAACAATTG
TTGAGGAAAAACATTCTATTGTTGATGGAAATGTGACTGATCCTATGGGA
GAGATGATTGAATTCCAATTAAAAAATGGTCAAAGTTTTACACATGATGA
TTACGTTTTGGTTGGAAATGATGGCAGTCAATTAAAAAATGGTGTGGCTC
TTGGTGGACCAAACAGTGATGGGGGAATTTTAAAAGATGTTACAGTGACT
TATGATAAGACATCTCAAACCATCAAAATCAATCATTTGAACTTAGGAAG
TGGACAAAAAGTAGTTCTTACCTATGATGTACGTTTAAAAGATAACTATA
TAAGTAACAAATTTTACAATACAAATAATCGTACAACGCTAAGTCCGAAG
AGTGAAAAAGAACCAAATACTATTCGTGATTTCCCAATTCCCAAAATTCG
TGATGTTCGTGAGTTTCCGGTACTAACCATCAGTAATCAGAAGAAAATGG
GTGAGGTTGAATTTATTAAAGTTAATAAAGACAAACATTCAGAATCGCTT
TTGGGAGCTAAGTTTCAACTTCAGATAGAAAAAGATTTTTCTGGGTATAA
GCAATTTGTTCCAGAGGGAAGTGATGTTACAACAAAGAATGATGGTAAAA
TTTATTTTAAAGCACTTCAAGATGGTAACTATAAATTATATGAAATTTCA
AGTCCAGATGGCTATATAGAGGTTAAAACGAAACCTGTTGTGACATTTAC
AATTCAAAATGGAGAAGTTACGAACCTGAAAGCAGATCCAAATGCTAATA
AAAATCAAATCGGGTATCTTGAAGGAAATGGTAAACATCTTATTACCAAC
ACTCCCAAACGCCCACCAGGTGTTTTTCCTAAAACAGGGGGAATTGGTAC
AATTGTCTATATATTAGTTGGTTCTACTTTTATGATACTTACCATTTGTT
CTTTCCGTCGTAAACAATTG

SEQ ID NO. 11

MKKRQKIWRGLSVTLLTLSQIPFGILVQGETQDTNQALGKVIVKKTGDNA
TPLGKATFVLKNDNDKSETSHETVEGSGEATFENIKPGDYTLREETAPIG
YKKTDKTWKVKVADNGATIIEGMDADKAEKRKEVLNAQYPKSAIYEDTKE
NYPLVNVEGSKVGEQYKALNPINGKDGRREIAEGWLSKKITGVNDLDKNK
YKIELTVEGKTTVETKELNQPLDVVVLLDNSNSMNNEPANNSQRALKAGE
AVEKLIDKITSNKDNRVALVTYASTIFDGTEATVSKGVADQNGKALNDSV
SWDYHKTTFTATTHNYSYLNLTNDANEVNILKSRIPKEAEHTNGDRTLYQ
FGATFTQKALMKANEILETQSSNARKKLIFHVTDGVPTMSYAINFNPYIS
TSYQNQFNSFLNKIPDRSGILQEDFIINGDDYQIVKGDGESFKLFSDRKV
PVTGGTTQAAYRVPQNQLSVMSNEGYAINSGYIYLYWRDYNWVYPFDPKT
KKVSATKQIKTHGEPTTLYFNGNIRPKGYDIFTVGIGVNGDPGATPLEAE
KFMQSISSKTENYTNVDDTNKIYDELNKYFKTIVEEKHSIVDGNVTDPMG
EMIEFQLKNGQSFTHDDYVLVGNDGSQLKNGVALGGPNSDGGILKDVTVT
YDKTSQTIKINHLNLGSGQKVVLTYDVRLKDNYISNKFYNTNNRTTLSPK
SEKEPNTIRDFPIPKIRDVREFPVLTISNQKKMGEVEFIKVNKDKHSESL
LGAKFQLQIEKDFSGYKQFVPEGSDVTTKNDGKIYFKALQDGNYKLYEIS
SPDGYIEVKTKPVVTFTIQNGEVTNLKADPNANKNQIGYLEGNGKHLITN
TPKRPPGVFPKTGGIGTTVYILVGSTFMILITCSFRRKQL

GBS 104 contains an N-terminal leader or signal sequence region which is indicated by the underlined sequence at the beginning of SEQ ID NO 11 above. In one embodiment, one or more amino acid sequences from the leader or signal sequence region of GBS 104 are removed. An example of such a GBS 104 fragment is set forth below as SEQ ID NO 12.

SEQ ID NO 12

GETQDTNQALGKVIVKKTGDNATPLGKATFVLKNDNDKSETSHETVEGSG
EATFENIKPGDYTLREETAPIGYKKTDKTWKVKVADNGATIIEGMDADKA
EKRKEVLNAQYPKSAIYEDTKENYPLVNVEGSKVGEQYKALNPINGKDGR
REIAEGWLSKKITGVNDLDKNKYKIELTVEGKTTVETKELNQPLDVVVLL
DNSNSMNNERANNSQRALKAGEAVEKLIDKITSNKDNRVALVTYASTIFD
GTEATVSKGVADQNGKALNDSVSWDYHKTTFTATTHNYSYLNLTNDANEV
NILKSRIPKEAEHINGDRTLYQFGATFTQKALMKANEILETQSSNARKKL
IFHVTDGVPTMSYAINFNPYISTSYQNQFNSFLNKIPDRSGILQEDFIIN
GDDYQIVKGDGESFKLFSDRKVPVTGGTTQAAYRVPQNQLSVMSNEGYAI
NSGYIYLYWRDYNWVYPFDPKTKKVSATKQIKTHGEPTTLYFNGNIRPKG
YDIFTVGIGVNGDPGATPLEAEKFMQSISSKTENYTNVDDTNKIYDELNK
YFKTIVEEKHSIVDGNVTDPMGEMIEFQLKNGQSFTHDDYVLVGNDGSQL
KNGVALGGPNSDGGILKDVTVTYDKTSQTIKINHLNLGSGQKVVLTYDVR
LKDNYISNKFYNTNNRTTLSPKSEKEPNTIRDFPIPKIRDVREFPVLTIS
NQKKMGEVEFIKVNKDKHSESLLGAKFQLQIEKDFSGYKQFVPEGSDVTT
KNDGKIYFKALQDGNYKLYEISSPDGYIEVKTKPVVTFTIQNGEVTNLKA
DPNANKNQIGYLEGNGKHLITNTPKRPPGVFPKTGGIGTIVYILVGSTFM
ILTICSFRRKQL

GBS 104 contains a C-terminal transmembrane and/or cytoplasmic region which is indicated by the underlined region near the end of SEQ ID NO 11 above. In one embodiment, one or more amino acids from the transmembrane or cytoplasmic regions are removed. An example of such a GBS 104 fragment is set forth below as SEQ ID NO 13.

SEQ ID NO: 13

MKKRQKIWRGLSVTLLILSQIPFGILVQGETQDTNQALGKVIVKKTGDNA
TPLGKATFVLKNDNDKSETSHETVEGSGEATFENIKPGDYTLREETAPIG
YKKTDKTWKVKVADNGATIIEGMDADKAEKRKEVLNAQYPKSAIYEDTKE
NYPLVNVEGSKVGEQYKALNPINGKDGRREIAEGWLSKKITGVNDLDKNK
YKIELTVEGKTTVETKELNQPLDVVVLLDNSNSMNNERANNSQRALKAGE
AVEKLIDKITSNKDNRVALVTYASTIFDGTEATVSKGVADQNGKALNDSV
SWDYHKTTFTATTHNYSYLNLTNDANEVNILKSRIPKEAEHINGDRTLYQ
FGATFTQKALMKANEILETQSSNARKKLIFHVTDGVPTMSYAINFNPYIS
TSYQNQFNSFLNKIPDRSGILQEDFIINGDDYQIVKGDGESFKLFSDRKV
PVTGGTTQAAYRVPQNQLSVMSNEGYAINSGYIYLYWRDYNWVYPFDPKT
KKVSATKQIKTHGEPTTLYFNGNIRPKGYDIFTVGIGVNGDPGATPLEAE
KFMQSISSKTENYTNVDDTNKIYDELNKYFKTIVEEKHSIVDGNVTDPMG
EMIEFQLKNGQSFTHDDYVLVGNDGSQLKNGVALGGPNSDGGILKDVTVT
YDKTSQTIKINHLNLGSGQKVVLTYDVRLKDNYISNKFYNTNNRTTLSPK
SEKEPNTIRDFPIPKIRDVREFPVLTISNQKKMGEVEFIKVNKDKHSESL
LGAKFQLQIEKDFSGYKQFVPEGSDVTTKNDGKTYFKALQDGNYKLYEIS
SPDGYIEVKTKPVVTFTIQNGEVTNLKADFNANKNQIGYLEGNGKHLITN
T

In one embodiment, one or more amino acids from the leader or signal sequence region and one or more amino acids from the transmembrane or cytoplasmic regions are removed. An example of such a GBS 104 fragment is set forth below as SEQ ID NO 14.

SEQ ID NO: 14

GETQDTNQALGKVIVKKTGDNATPLGKATFVLKNDNDKSETSHETVEGSG
EATFENIKPGDYTLREETAPIGYKKTDKTWKVKVADNGATIIEGMDADKA
EKRKEVLNAQYPKSAIYEDTKENYPLVNVEGSKVGEQYKALNPINGKDGR
REIAEGWLSKKITGVNDLDKNKYKIELTVEGKTTVETKELNQPLDVVVLL
DNSNSMNNERANNSQRALKAGEAVEKLIDKITSNKDNRVALVTYASTIFD
GTEATVSKGVADQNGKALNDSVSWDYHKTTFTATTHNYSYLNLTNDANEV
NILKSRIPKEAEHINGDRTLYQFGATFTQKALMKANEILETQSSNARKKL
IFHVTDGVPTMSYAINFNPYISTSYQNQFNSFLNKIPDRSGILQEDFIIN
GDDYQIVKGDGESFKLFSDRKVPVTGGTTQAAYRVPQNQLSVMSNEGYAI
NSGYIYLYWRDYNWVYPFDPKTKKVSATKQIKTHGEPTTLYFNGNIRPKG
YDIFTVGIGVNGDPGATPLEAEKFMQSISSKTENYTNVDDTNKIYDELNK
YFKTIVEEKHSIVDGNVTDPMGEMIEFQLKNGQSFTHDDYVLVGNDGSQL
KNGVALGGPNSDGGILKDVTVTYDKTSQTIKINHLNLGSGQKVVLTYDVR
LKDNYISNKFYNTNNRTTLSPKSEKEPNTIRDFPIPKIRDVREFPVLTIS
NQKKMGEVEFIKVNKDKHSESLLGAKFQLQIEKDFSGYKQFVPEGSDVTT
KNDGKIYFKALQDGNYKLYEISSPDGYIEVKTKPVVTFTIQNGEVTNLKA
DPNANKNQIGYLEGNGKHLITNT

GBS 104, like GBS 80, contains an amino acid motif indicative of a cell wall anchor: SEQ ID NO:123 FPKTG (shown in italics in SEQ ID NO:11 above). In some recombinant host cell systems, it may be preferable to remove this motif to facilitate secretion of a recombinant GBS 104 protein from the host cell. Accordingly, in one preferred fragment of GBS 104 for use in the invention, only the transmembrane and/or cytoplasmic regions and the cell wall anchor motif are removed from GBS 104. Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Two pilin motifs, containing conserved lysine (K) residues, have been identified in GBS 104. The pilin motif sequences are underlined in SEQ ID NO:11, below. Conserved lysine (K) residues are marked in bold, at amino acid residues 141 and 149 and at amino acid residues 499 and 507. The pilin sequence, in particular the conserved lysine residues, are thought to be important for the formation of oligomeric, pilus-like structures of GBS 104. Preferred fragments of GBS 104 include at least one conserved lysine residue. Preferably, fragments include at least one pilin sequence.

SEQ ID NO. 11

MKKRQKIWRGLSVTLLILSQIPFGILVQGETQDTNQALGKVIVKKTGDNA
TPLGKATFVLKNDNDKSETSHETVEGSGEATFENIKPGDYTLREETAPIG
YKKTDKTWKVKVADNGATIIEGMDADKAEKRKEVLNAQYPKSAIYEDTKE
NYPLVNVEGSKVGEQYKALNPINGKDGRREIAEGWLSKKITGVNDLDKNK
YKIELTVEGKTTVETKELNQPLDVVVLLDNSNSMNNERANNSQRALKAGE
AVEKLIDKITSNKDNRVALVTYASTIFDGTEATVSKGVADQNGKALNDSV
SWDYHKTTFTATTHNYSYLNLTNDANEVNILKSRIPKEAEHINGDRTLYQ
FGATFTQKALMKANEILETQSSNARKKLIFHVTDGVPTMSYAINFNPYIS
TSYQNQFNSFLNKIPDRSGILQEDFIINGDDYQIVKGDGESFKLFSDRKV
PVTGGTTQAAYRVPQNQLSVMSNEGYAINSGYIYLYWRDYNWVYPFDPKT
KKVSATKQIKTHGEPTTLYFNGNIRPKGYDIFTVGIGVNGDPGATPLEAE
KFMQSISSKTENYTNVDDTNKIYDELNKYFKTIVEEKHSIVDGNVTDPMG
EMIEFQLKNGQSFTHDDYVLVGNDGSQLKNGVALGGPNSDGGILKDVTVT
YDKTSQTIKINHLNLGSGQKVVLTYDVRLKDNYISNKFYNTNNRTTLSPK
SEKEPNTIRDFPIPKIRDVREFPVLTISNQKKMGEVEFIKVNKDKHSESL
LGAKFQLQIEKDFSGYKQFPFEGSDVTTKNDGKIYFKALQDGNYKLYEIS
SPDGYIEVKTKPVVTFTIQNGEVTNLKADPNANKNQIGYLEGNGKHLITN
TPKRPPGVFPKTGGIGTIVYILVGSTFMILTICSFRRKQL

Two E boxes containing a conserved glutamic residues have also been identified in GBS 104. The E box motifs are underlined in SEQ ID NO:11 below. The conserved glutamic acid (E) residues, at amino acid residues 94 and 798, are marked in bold. The E box motifs, in particular the conserved glutamic acid residues, are thought to be important for the formation of oligomeric pilus-like structures of GBS 104. Preferred fragments of GBS 104 include at least one conserved glutamic acid residue. Preferably, fragments include at least one E box motif.

SEQ ID NO. 11

MKKRQKIWRGLSVTLLILSQIPFGILVQGETQDTNQALGKVIVKKTGDNA
TPLGKATFVLKNDNDKSETSHETVEGSGEATFENIKPGDYTLREETAPIG
YKKTDKTWKVKVADNGATIIEGMDADKAEKRKEVLNAQYPKSAIYEDTKE
NYPLVNVEGSKVGEQYKALNPINGKDGRREIAEGWLSKKITGVNDLDKNK
YKIELTVEGKTTVETKELNQPLDVVVLLDNSNSMNNERANNSQRALKAGE
AVEKLIDKITSNKDNRVALVTYASTIFDGTEATVSKGVADQNGKALNDSV
SWDYHKTTFTATTHNYSYLNLTNDANEVNILKSRIPKEAEHINGDRTLYQ
FGATFTQKALMKANEILETQSSNARKKLIFHVTDGVPTMSYAINFNPYIS
TSYQNQFNSFLNKIPDRSGILQEDFIINGDDYQIVKGDGESFKLFSDRKV
PVTGGTTQAAYRVPQNQLSVMSNEGYAINSGYIYLYWRDYNWVYPFDPKT
KKVSATKQIKTHGEPTTLYFNGNIRPKGYDIFTVGIGVNGDPGATPLEAE
KFMQSISSKTENYTNVDDTNKIYDELNKYFKTIVEEKHSIVDGNVTDPMG
EMIEFQLKNGQSFTHDDYVLVGNDGSQLKNGVALGGPNSDGGILKDVTVT
YDKTSQTIKINHLNLGSGQKVVLTYDVRLKDNYISNKFYNTNNRTTLSPK
SEKEPNTIRDFPIPKIRDVREFPVLTISNQKKMGEVEFIKVNKDKHSESL
LGAKFQLQIEKDFSGYKQFVPEGSDVTTKNDGKIYFKALQDGNYKLYEIS
SPDGYIEVKTKPVVTFTIQNGEVTNLKADPNANKNQIGYLEGNGKHLITN
TPKRPPGVFPKTGGIGTIVYILVGSTFMILTICSFRRKQL

### GBS 067

The following offers examples of preferred GBS 067 fragments. Nucleotide and amino acid sequence of GBS 067 sequences from serotype V isolated strain 2603 are set forth below as SEQ ID NOS: 15 and 16.

SEQ ID NO: 15

ATGAGAAAATACCAAAAATTTTCTAAAATATTGACGTTAAGTCTTTTTTG
TTTGTCGCAAATACCGCTTAATACCAATGTTTTAGGGGAAAGTACCGTAC
CGGAAAATGGTGCTAAAGGAAAGTTAGTTGTTAAAAAGACAGATGACCAG
AACAAACCACTTTCAAAAGCTACCTTTGTTTTAAAAACTACTGCTCATCC
AGAAAGTAAAATAGAAAAAGTAACTGCTGAGCTAACAGGTGAAGCTACTT
TTGATAATCTCATACCTGGAGATTATACTTTATCAGAAGAAACAGCGCCC
GAAGGTTATAAAAAGACTAACCAGACTTGGCAAGTTAAGGTTGAGAGTAA
TGGAAAAACTACGATACAAAATAGTGGTGATAAAAATTCCACAATTGGAC
AAAATCAGGAAGAACTAGATAAGCAGTATCCCCCCACAGGAATTTATGAA
GATACAAAGGAATCTTATAAACTTGAGCATGTTAAAGGTTCAGTTCCAAA
TGGAAAGTCAGAGGCAAAAGCAGTTAACCCATATTCAAGTGAAGGTGAGC
ATATAAGAGAAATTCCAGAGGGAACATTATCTAAACGTATTTCAGAAGTA
GGTGATTTAGCTCATAATAAATATAAAATTGAGTTAACTGTCAGTGGAAA
AACCATAGTAAAACCAGTGGACAAACAAAAGCCGTTAGATGTTGTCTTCG
TACTCGATAATTCTAACTCAATGAATAACGATGGCCCAAATTTTCAAAGG
CATAATAAAGCCAAGAAAGCTGCCGAAGCTCTTGGGACCGCAGTAAAAGA
TATTTTAGGAGCAAACAGTGATAATAGGGTTGCATTAGTTACCTATGGTT
CAGATATTTTTGATGGTAGGAGTGTAGATGTCGTAAAAGGATTTAAAGAA
GATGATAAATATTATGGCCTTCAAACTAAGTTCACAATTCAGACAGAGAA
TTATAGTCATAAACAATTAACAAATAATGCTGAAGAGATTATAAAAAGGA
TTCCGACAGAAGCTCCTAAAGCTAAGTGGGGATCTACTACCAATGGATTA
ACTCCAGAGCAACAAAAGGAGTACTATCTTAGTAAAGTAGGAGAAACATT
TACTATGAAAGCCTTCATGGAGGCAGATGATATTTTGAGTCAAGTAAATC
GAAATAGTCAAAAAATTATTGTTCATGTAACTGATGGTGTTCCTACGAGA
TCATATGCTATTAATAATTTTAAACTGGGTGCATCATATGAAAGCCAATT
TGAACAAATGAAAAAAAATGGATATCTAAATAAAAGTAATTTTCTACTTA
CTGATAAGCCCGAGGATATAAAAGGAAATGGGGAGAGTTACTTTTTGTTT
CCCTTAGATAGTTATCAAACACAGATAATCTCTGGAAACTTACAAAAACT
TCATTATTTAGATTTAAATCTTAATTACCCTAAAGGTACAATTTATCGAA
ATGGACCAGTGAAAGAACATGGAACACCAACCAAACTTTATATAAATAGT
TTAAAACAGAAAAATTATGACATTTTTAATTTTGGTATCGATATATCTGG
TTTTAGACAAGTTTATAATGAGGAGTATAAGAAAAATCAAGATGGTACTT
TTCAAAAATTGAAAGAGGAAGCTTTTAAACTTTCAGATGGAGAAATCACA
GAACTAATGAGGTCGTTCTCTTCCAAACCTGAGTACTACACCCCTATCGT
AACTTCAGCCGATACATCTAACAATGAAATTTTATCTAAAATTCAGCAAC
AATTTGAAACGATTTTAACAAAAGAAAACTCAATTGTTAATGGAACTATC
GAAGATCCTATGGGTGATAAAATCAATTTACAGCTTGGTAATGGACAAAC
ATTACAGCCAAGTGATTATACTTTACAGGGAAATGATGGAAGTGTAATGA
AGGATGGTATTGCAACTGGTGGGCCTAATAATGATGGTGGAATACTTAAG
GGGGTTAAATTAGAATACATCGGAAATAAACTCTATGTTAGAGGTTTGAA
TTTAGGAGAAGGTCAAAAAGTAACACTCACATATGATGTGAAACTAGATG
ACAGTTTTATAAGTAACAAATTCTATGACACTAATGGTAGAACAACATTG
AATCCTAAGTCAGAGGATCCTAATACACTTAGAGATTTTCCAATCCCTAA
AATTCGTGATGTGAGAGAATATCCTACAATAACGATTAAAAACGAGAAGA
AGTTAGGTGAAATTGAATTTATAAAAGTTGATAAAGATAATAATAAGTTG
CTTCTCAAAGGAGCTACGTTTGAACTTCAAGAATTTAATGAAGATTATAA
ACTTTATTTACCAATAAAAAATAATAATTCAAAAGTAGTGACGGGAGAAA
ACGGCAAAATTTCTTACAAAGATTTGAAAGATGGCAAATATCAGTTAATA
GAAGCAGTTTCGCCGGAGGATTATCAAAAAATTACTAATAAACCAATTTT
AACTTTTGAAGTGGTTAAAGGATCGATAAAAAATATAATAGCTGTTAATA
AACAGATTTCTGAATATCATGAGGAAGGTGACAAGCATTTAATTACCAAC
ACGCATATTCCACCAAAAGGAATTATTCCTATGACAGGTGGGAAAGGAAT
TCTATCTTTCATTTTAATAGGTGGAGCTATGATGTCTATTGCAGGTGGAA
TTTATATTTGGAAAAGGTATAAGAAATCTAGTGATATGTCCATCAAAAAA
GAT

SEQ ID NO: 16

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGTATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGIIPMTGGKGILSFILIGGAMMSIAGGIYIWKRYKKSSDMSIKK
D

GBS 067 contains a C-terminus transmembrane region which is indicated by the underlined region closest to the C-terminus of SEQ ID NO:16 above. In one embodiment, one or more amino acids from the transmembrane region is removed and or the amino acid is truncated before the transmembrane region. An example of such a GBS 067 fragment is set forth below as SEQ ID NO:17.

SEQ ID NO: 17

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGIIPMTGGKGILS

GBS 067 contains an amino acid motif indicative of a cell wall anchor (an LPXTG (SEQ ID NO:122) motif): SEQ ID NO:18 I PMTG. (shown in italics in SEQ ID NO:16 above). In some recombinant host cell systems, it may be preferable to remove this motif to facilitate secretion of a recombinant GBS 067 protein from the host cell. Accordingly, in one preferred fragment of GBS 067 for use in the invention, the transmembrane and the cell wall anchor motif are removed from GBS 67. An example of such a GBS 067 fragment is set forth below as SEQ ID NO:19.

SEQ ID NO: 19

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGI

Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Three pilin motifs, containing conserved lysine (K) residues have been identified in GBS 67. The pilin motif sequences are underlined in SEQ ID NO:16, below. Conserved lysine (K) residues are marked in bold, at amino acid residues 478 and 488, at amino acid residues 340 and 342, and at amino acid residues 703 and 717. The pilin sequences, in particular the conserved lysine residues, are thought to be important for the formation of oligomeric, pilus-like structures of GBS 67. Preferred fragments of GBS 67 include at least one conserved lysine residue. Preferably, fragments include at least one pilin sequence.

SEQ ID NO: 16

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGIIPMTGGKGILSFILIGGAMMSIAGGIYIWKRYKKSSDMSIKK
D

Two E boxes containing conserved glutamic residues have also been identified in GBS 67. The E box motifs are underlined in SEQ ID NO:16 below. The conserved glutamic acid (E) residues, at amino acid residues 96 and 801, are marked in bold. The E box motifs, in particular the conserved glutamic acid residues, are thought to be important for the formation of oligomeric pilus-like structures of GBS 67. Preferred fragments of GBS 67 include at least one conserved glutamic acid residue. Preferably, fragments include at least one E box motif.

SEQ ID NO: 16

MRKYQKFSKILTLSLFCLSQIPLNTNVLGESTVPENGAKGKLVVKKTDDQ
NKPLSKATFVLKTTAHPESKIEKVTAELTGEATFDNLIPGDYTLSEETAP
EGYKKTNQTWQVKVESNGKTTIQNSGDKNSTIGQNQEELDKQYPPTGIYE
DTKESYKLEHVKGSVPNGKSEAKAVNPYSSEGEHIREIPEGTLSKRISEV
GDLAHNKYKIELTVSGKTIVKPVDKQKPLDVVFVLDNSNSMNNDGPNFQR
HNKAKKAAEALGTAVKDILGANSDNRVALVTYGSDIFDGRSVDVVKGFKE
DDKYYGLQTKFTIQTENYSHKQLTNNAEEIIKRIPTEAPKAKWGSTTNGL
TPEQQKEYYLSKVGETFTMKAFMEADDILSQVNRNSQKIIVHVTDGVPTR
SYAINNFKLGASYESQFEQMKKNGYLNKSNFLLTDKPEDIKGNGESYFLF
PLDSYQTQIISGNLQKLHYLDLNLNYPKGTIYRNGPVKEHGTPTKLYINS
LKQKNYDIFNFGIDISGFRQVYNEEYKKNQDGTFQKLKEEAFKLSDGEIT
ELMRSFSSKPEYYTPIVTSADTSNNEILSKIQQQFETILTKENSIVNGTI
EDPMGDKINLQLGNGQTLQPSDYTLQGNDGSVMKDGIATGGPNNDGGILK
GVKLEYIGNKLYVRGLNLGEGQKVTLTYDVKLDDSFISNKFYDTNGRTTL
NPKSEDPNTLRDFPIPKIRDVREYPTITIKNEKKLGEIEFIKVDKDNNKL
LLKGATFELQEFNEDYKLYLPIKNNNSKVVTGENGKISYKDLKDGKYQLI
EAVSPEDYQKITNKPILTFEVVKGSIKNIIAVNKQISEYHEEGDKHLITN
THIPPKGIIPMTGGKGILSFILIGGAMMSIAGGIYIWKRYKKSSDMSIKK
D

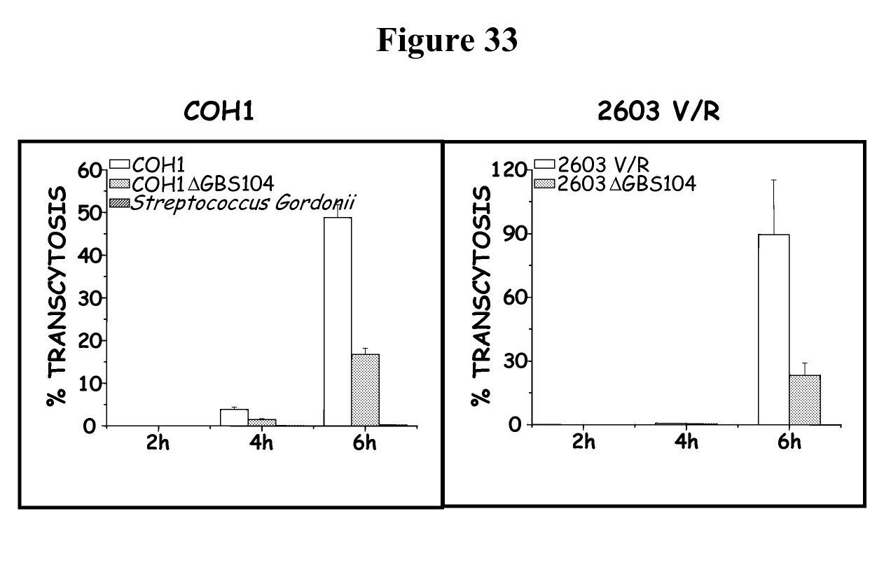

Predicted secondary structure for the GBS 067 amino acid sequence is set forth in FIG. 33. As shown in this FIG., GBS 067 contains several regions predicted to form alpha helical structures. Such alpha helical regions are likely to form coiled-coil structures and may be involved in oligomerization of GBS 067.

The amino acid sequence for GBS 067 also contains a region which is homologous to the Cna_B domain of the Staphylococcus aureus collagen-binding surface protein (pfam05738). Although the Cna_B region is not thought to mediate collagen binding, it is predicted to form a beta sandwich structure. In the Staph aureus protein, this beta sandwich structure is through to form a stalk that presents the ligand binding domain away from the bacterial cell surface. This same amino acid sequence region is also predicted to be an outer membrane protein involved in cell envelope biogenesis.

The amino acid sequence for GBS 067 contains a region which is homologous to a von Willebrand factor (vWF) type A domain. The vWF type A domain is present at amino acid residues 229-402 of GBS 067 as shown in SEQ ID NO:16. This type of sequence is typically found in extracellular proteins such as integrins and it thought to mediate adhesion, including adhesion to collagen, fibronectin, and fibrinogen, discussed above.

Because applicants have identified GBS 67 as a surface exposed protein on GBS and because GBS 67 may be involved in GBS adhesion, the immunogenicity of the GBS 67 protein was examined in mice. The results of an immunization assay with GBS 67 are set forth in Table 48, below.

TABLE 48
GBS 67 Protects Mice in an Immunization Assay

| Challenge | GBS 67 immungen | | PBS immunogen | | |
|---|---|---|---|---|---|
| GBS strain | dead/ | | dead/ | | FACS |
| (serotype) | treated | % survival | treated | % survival | Δmean |
| 3050 (II) | 0/30 | 100 | 29/49 | 41 | 460 |
| CJB111 (V) | 76/185 | 59 | 143/189 | 24 | 481 |
| 7357 b (Ib) | 34/56 | 39 | 65/74 | 12 | 316 |

As shown in Table 48, immunization with GBS 67 provides a substantially improved survival rate for challenged mice relative to negative control, PBS, immunized mice. These results indicate that GBS 67 may comprise an immunogenic composition of the invention.

### GBS 59

The following offers examples of GBS 59 fragments. Nucleotide and amino acid sequences of GBS 59 sequenced from serotype V isolated strain 2603 are set forth below as SEQ ID NOS: 125 and 126. The GBS 59 polypeptide of SEQ ID NO:126 is referred to as SAG1407.

SEQ ID NO: 125

TTAAGCTTCCTTTGATTGGCGTCTTTTCATGATAACTACTGCTCCAAGCA
TAATGCTTAAACCAATAATTGTGAAAAGAATTGTACCAATACCACCTGTT
TGTGGGATTGTTACCTTTTTATTTTCTACACGTGTCGCATCTTTTTGGTT
GCTGTTAGCAACGTAGTCAATGTTACCACCTGTTATGTATGACCCTTGAT
TAACTACAAACTTAATATTACCTGCCAACTTAGCAAATCCTGCTGGAGCA
AGTGTTTCTTCAAGGTTGTAAGTACCGTCTGCAAGACCTGTAACTTCAAA
TTGACCTTGATCGTTTGAAGTGTAGGTAATGGCTCTAGCCTTATCTGTTA
TCCACTCATAAGCTGTACGAGCCTCAATGAAGGCTGCATCGTAATCTGCT
TGTTTAGTTTTGATAAGTTCTTTTGCAGTAATTCCTTTTTCACCTTTTTG
GTCTGTTGCAGACAACTTGTTATAAGCAGCGATAGCTTCATCTAAAGCTA
TTTTCTTAGCAGCTAAAGTTTTTTGACCTTCTGATTGATCTGCTTTAAGA
GCAAGGTATTTACCTGCTGAGTTTTTCACAACGAATTGTGCACCAGCCAA
ACGGTCACCTTGTTCATTAGTTTTGACAAATTTCTTACCATGAGTTTCAA
CTTTTGGTTCAGTTGGGTTCAATGGTGTTGGGTTATCAGAATCTTTGGTA
TTGGTAATGGTTACTTTACCATTTTCTAGATTTATTGCACTTCCGTAACC
AGAAACACGTTCTGAGATCATGTATGATTTGTTTTCTAGACCAGTGAATT
TACCCGAGAAGTTACCAGATACTTCAAATTTGATACCATTTCCAAGGTCG
ATTGTACCTTTAGATGTTTTTGTCAATGATACTGAAGCAACAGTTTTATC
TTTATCTTTCAATGTGTAAACAACGTTTACACCATCAGGTGCAATTCCGT
CAGACCAAGTTTTAGCAACTGTTACTTCACCCTTTGAAGGTGTAACAGGA
AGTTCAGTCAAGTCTTTACCTGGTTTGTTACCATACGACAATTTGATATC
ATTGGATTCTGGATTATCAATAATTGCTTGACCATTAACAGTAGCACTAT
AAGTCAATGTAAATTCAATATCAGCTGTTTTAGCTGCTTTTTCCAATTTG
CCCAATCCATCAGCTGTGAATTTTAATGTGAAACCACGGGCATCAATGCT
AAGTTCATAGTCTGTATCCTTAGCAAAAGTTTCTGTAGTTCCTGAAGCTT
TAAGGCTAACAGTTGAACCCATTGTCAAACCATTTGACATTATATCTGTC
CAAACCAAGTTTTCGTATTTAGAACCTTTGTGAATTTTTGTTTTAACTTC
ATAAGGAACAACTTTACCGATTTCAGCAGTAGCAGTTGCTTTGTCACGTG
CATAATTACCATAATTTGCGCCAGCTGTCAAAAGTCTATTAACATCTGTC
AATGCTGTCAAATCGTTTGTTTTAGCAAAGTTTTTATCAATTTCTGGTTT
TTCTTCAGTGTTCTTTGGATAAACATGGGCATCAGCAACAACACCATCTT
CATTTACCAATGGAAGAGTGATGTTAACTGGAACCGCTTTTGAAGCAGCC
AGGAGGGAACCATTATTGTTGTAAGTAGATTTTGATTTAACTTCAACAAT
TTTAAACTCGCCTTTCAATCCTTTGGTGTTGAAAACAAGTCCAGTATCTC
CCTCTGGTGTCAATCCAGACACGGCCTCATCAATATTTACTGTTATTTCA
GGAGTACCATCTTTATTAATTAAGGCTGGTGTTAATTTGTTACCTTCTTT
TGCCTTAACATATTGCACTTTACCACTTTTATCTTCTTTCAAAGCTAAAG
CAAAGAACGCACCTTCGATTTCTTTAGATCCCTCGCCAAAGTAACCAGCA
AGGTCAGAAATAGCTCCACCTTTGTAGTCTTTTCCGTTAAGACCTGTAGT
TCCTGGGAAGTTACTTTTGTTAAGATTTGATTCGGTTTGCAAAATCTTGT
GCAAAGTCACTGTATTAGTTGTTGCTTCATCCGCAAACGCTGGTGCAACT
GAGAGCAATGACGTTAAAGTCAGTAACAATGCCGAGAACATTGCAAAATA
TTTGTTGATTCTTTTCAT

SEQ ID NO: 126

MKRINKYFAMFSALLLTLTSLLSVAPAFADEATTNTVTLHKILQTESNLN
KSNFPGTTGLNGKDYKGGAISDLAGYFGEGSKEIEGAFFALALKEDKSGK
VQYVKAKEGNKLTPALINKDGTPEITVNIDEAVSGLTPEGDTGLVFNTKG
LKGEFKIVEVKSKSTYNNNGSLLAASKAVPVNITLPLVNEDGVVADAHVY
PKNTEEKPEIDKNFAKTNDLTALTDVNRLLTAGANYGNYARDKATATAEI
GKVVPYEVKTKIHKGSKYENLVWTDIMSNGLTMGSTVSLKASGTTETFAK
DTDYELSIDARGFTLKFTADGLGKLEKAAKTADIEFTLTYSATVNGQAII
DNPESNDIKLSYGNKPGKDLTELPVTPSKGEVTVAKTWSDGIAPDGVNVV
YTLKDKDKTVASVSLTKTSKGTIDLGNGIKFEVSGNFSGKFTGLENKSYM
ISERVSGYGSAINLENGKVTITNTKDSDNPTPLNPTEPKVETHGKKFVKT
NEQGDRLAGAQFVVKNSAGKYLALKADQSEGQKTLAAKKIALDEAIAAYN
KLSATDQKGEKGITAKELIKTKQADYDAAFIEARTAYEWITDKARAITYT
SNDQGQFEVTGLADGTYNLEETLAPAGFAKLAGNIKFVVNQGSYITGGNI
DYVANSNQKDATRVENKKVTIPQTGGIGTILFTIIGLSIMLGAVVIMKRR
QSKEA

Nucleotide and amino acid sequences of GBS 59 sequenced from serotype V isolated strain CJB111 are set forth below as SEQ ID NOS: 127 and 128. The GBS 59 polypeptide of SEQ ID NO:128 is referred to as BO1575.

SEQ ID NO: 127

ATGAAAAAAATCAACAAATGTCTTACAATGTTCTCGACACTGCTATTGAT
CTTAACGTCACTATTCTCAGTTGCACCAGCGTTTGCGGACGACGCAACAA
CTGATACTGTGACCTTGCACAAGATTGTCATGCCACAAGCTGCATTTGAT
AACTTTACTGAAGGTACAAAAGGTAAGAATGATAGCGATTATGTTGGTAA
ACAAATTAATGACCTTAAATCTTATTTTGGCTCAACCGATGCTAAAGAAA
TCAAGGGTGCTTTCTTTGTTTTCAAAAATGAAACTGGTACAAAATTCATT
ACTGAAAATGGTAAGGAAGTCGATACTTTGGAAGCTAAAGATGCTGAAGG
TGGTGCTGTTCTTTCAGGGTTAACAAAAGACAATGGTTTTGTTTTTAACA
CTGCTAAGTTAAAAGGAATTTACCAAATCGTTGAATTGAAAGAAAAATCA
AACTACGATAACAACGGTTCTATCTTGGCTGATTCAAAAGCAGTTCCAGT
TAAAATCACTCTGCCATTGGTAAACAACCAAGGTGTTGTTAAAGATGCTC
ACATTTATCCAAAGAATACTGAAACAAAACCACAAGTAGATAAGAACTTT
GCAGATAAAGATCTTGATTATACTGACAACCGAAAAGACAAAGGTGTTGT
CTCAGCGACAGTTGGTGACAAAAAAGAATACATAGTTGGAACAAAAATTC
TTAAAGGCTCAGACTATAAGAAACTGGTTTGGACTGATAGCATGACTAAA
GGTTTGACGTTCAACAACAACGTTAAAGTAACATTGGATGGTGAAGATTT
TCCTGTTTTAAACTACAAACTCGTAACAGATGACCAAGGTTTCCGTCTTG
CCTTGAATGCAACAGGTCTTGCAGCAGTAGCAGCAGCTGCAAAAGACAAA
GATGTTGAAATCAAGATCACTTACTCAGCTACGGTGAACGGCTCCACTAC
TGTTGAAATTCCAGAAACCAATGATGTTAAATTGGACTATGGTAATAACC
CAACGGAAGAAAGTGAACCACAAGAAGGTACTCCAGCTAACCAAGAAATT
AAAGTCATTAAAGACTGGGCAGTAGATGGTACAATTACTGATGCTAATGT
TGCAGTTAAAGCTATCTTTACCTTGCAAGAAAAACAAACGGATGGTACAT
GGGTGAACGTTGCTTCACACGAAGCAACAAAACCATCACGCTTTGAACAT
ACTTTCACAGGTTTGGATAATGCTAAAACTTACCGCGTTGTCGAACGTGT
TAGCGGCTACACTCCAGAATACGTATCATTTAAAAATGGTGTTGTGACTA
TCAAGAACAACAAAAACTCAAATGATCCAACTCCAATCAACCCATCAGAA
CCAAAAGTGGTGACTTATGGACGTAAATTTGTGAAAACAAATCAAGCTAA
CACTGAACGCTTGGCAGGAGCTACCTTCCTCGTTAAGAAAGAAGGCAAAT
ACTTGGCACGTAAAGCAGGTGCAGCAACTGCTGAAGCAAAGGCAGCTGTA
AAAACTGCTAAACTAGCATTGGATGAAGCTGTTAAAGCTTATAACGACTT
GACTAAAGAAAAACAAGAAGGCCAAGAAGGTAAAACAGCATTGGCTACTG
TTGATCAAAAACAAAAAGCTTACAATGACGCTTTTGTTAAAGCTAACTAC
TCATATGAATGGGTTGCAGATAAAAAGGCTGATAATGTTGTTAAATTGAT
CTCTAACGCCGGTGGTCAATTTGAAATTACTGGTTTGGATAAAGGCACTT
ATGGCTTGGAAGAAACTCAAGCACCAGCAGGTTATGCGACATTGTCAGGT
GATGTAAACTTTGAAGTAACTGCCACATCATATAGCAAAGGGGCTACAAC
TGACATCGCATATGATAAAGGCTCTGTAAAAAAAGATGCCCAACAAGTTC
AAAACAAAAAAGTAACCATCCCACAAACAGGTGGTATTGGTACAATTCTT
TTCACAATTATTGGTTTAAGCATTATGCTTGGAGCAGTAGTTATCATGAA
AAAACGTCAATCAGAGGAAGCTTAA

SEQ ID NO: 128

MKKINKCLTMFSTLLLILTSLFSVAPAFADDATTDTVTLHKIVMPQAAFD
NFTEGTKGKNDSDYVGKQINDLKSYFGSTDAKEIKGAFFVFKNETGTKFI
TENGKEVDTLEAKDAEGGAVLSGLTKDNGFVFNTAKLKGIYQIVELKEKS
NYDNNGSILADSKAVPVKITLPLVNNQGVVKDAHIYPKNTETKPQVDKNF
ADKDLDYTDNRKDKGVVSATVGDKKEYIVGTKILKGSDYKKLVWTDSMTK
GLTFNNNVKVTLDGEDFPVLNYKLVTDDQGFRLALNATGLAAVAAAAKDK
DVEIKITYSATVNGSTTVEIPETNDVKLDYGNNPTEESEPQEGTPANQEI
KVIKDWAVDGTITDANVAVKAIFTLQEKQTDGTWVNVASHEATKPSRFEH
TFTGLDNAKTYRVVERVSGYTPEYVSFKNGVVTIKNNKNSNDPTPINPSE
PKVVTYGRKFVKTNQANTERLAGATFLVKKEGKYLARKAGAATAEAKAAV
KTAKLALDEAVKAYNDLTKEKQEGQEGKTALATVDQKQKAYNDAFVKANY
SYEWVADKKADNVVKLISNAGGQFEITGLDKGTYGLEETQAPAGYATLSG
DVNFEVTATSYSKGATTDIAYDKGSVKKDAQQVQNKKVTIPQTGGIGTIL
FTIIGLSIMLGAVVIMKKRQSEEA

The GBS 59 polypeptides contain an amino acid motif indicative of a cell wall anchor: SEQ ID NO:129 IPQTG (shown in italics in SEQ ID NOS: 126 and 128 above). In some recombinant host cell systems, it may be preferable to remove this motif to facilitate secretion of a recombinant GBS 59 protein from the host cell. Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Pilin motifs, containing conserved lysine (K) residues have been identified in the GBS 59 polypeptides. The pilin motif sequences are underlined in each of SEQ ID NOS: 126 and 128, below. Conserved lysine (K) residues are marked in bold. The conserved lysine (K) residues are located at amino acid residues 202 and 212 and amino acid residues 489 and 495 of SEQ ID NO:126 and at amino acid residues 188 and 198 of SEQ ID NO:128. The pilin sequences, in particular the conserved lysine residues, are thought to be important for the formation of oligomeric, pilus-like structures of GBS 59. Preferred fragments of GBS 59 include at least one conserved lysine residue. Preferably, fragments include at least one pilin sequence.

SEQ ID NO: 126

MKRINKYFAMFSALLLTLTSLLSVAPAFADEATTNTVTLHKILQTESNLN
KSNFPGTTGLNGKDYKGGAISDLAGYFGEGSKEIEGAFFALALKEDKSGK
VQYVKAKEGNKLTPALINKDGTPEITVNIDEAVSGLTPEGDTGLVFNTKG
LKGEFKIVEVKSKSTYNNNGSLLAASKAVPVNITLPLVNEDGVVADAHVY
PKNTEEKPEIDKNFAKTNDLTALTDVNRLLTAGANYGNYARDKATATAEI
GKVVPYEVKTKIHKGSKYENLVWTDIMSNGLTMGSTVSLKASGTTETFAK
DTDYELSIDARGFTLKFTADGLGKLEKAAKTADIEFTLTYSATVNGQAII
DNPESNDIKLSYGNKPGKDLTELPVTPSKGEVTVAKTWSDGIAPDGVNVV
YTLKDKDKTVASVSLTKTSKGTIDLGNGIKFEVSGNFSGKFTGLENKSYM
ISERVSGYGSAINLENGKVTITNTKDSDNPTPLNPTEPKVETHGKKFVKT
NEQGDRLAGAQFVVKNSAGKYLALKADQSEGQKTLAAKKIALDEAIAAYN
KLSATDQKGEKGITAKELIKTKQADYDAAFIEARTAYEWITDKARAITYT
SNDQGQFEVTGLADGTYNLEETLAPAGFAKLAGNIKFVVNQGSYITGGNI
DYVANSNQKDATRVENKKVTIPQTGGIGTILFTIIGLSIMLGAVVIMKRR
QSKEA

SEQ ID NO: 128

MKKINKCLTMFSTLLLILTSLFSVAPAFADDATTDTVTLHKIVMPQAAFD
NFTEGTKGKNDSDYVGKQINDLKSYFGSTDAKEIKGAFFVFKNETGTKFI
TENGKEVDTLEAKDAEGGAVLSGLTKDNGFVFNTAKLKGIYQIVELKEKS
NYDNNGSILADSKAVPVKITLPLVNNQGVVKDAHIYPKNTETKPQVDKNF
ADKDLDYTDNRKDKGVVSATVGDKKEYIVGTKILKGSDYKKLVWTDSMTK
GLTFNNNVKVTLDGEDFPVLNYKLVTDDQGFRLALNATGLAAVAAAAKDK
DVEIKITYSATVNGSTTVEIPETNDVKLDYGNNPTEESEPQEGTPANQEI
KVIKDWAVDGTITDANVAVKAIFTLQEKQTDGTWVNVASHEATKPSRFEH
TFTGLDNAKTYRVVERVSGYTPEYVSFKNGVVTIKNNKNSNDPTPINPSE
PKVVTYGRKFVKTNQANTERLAGATFLVKKEGKYLARKAGAATAEAKAAV
KTAKLALDEAVKAYNDLTKEKQEGQEGKTALATVDQKQKAYNDAFVKANY
SYEWVADKKADNVVKLISNAGGQFEITGLDKGTYGLEETQAPAGYATLSG
DVNFEVTATSYSKGATTDIAYDKGSVKKDAQQVQNKKVTIPQTGGIGTIL
FTIIGLSIMLGAVVIMKKRQSEEA

An E box containing a conserved glutamic residue has also been identified in each of the GBS 59 polypeptides. The E box motif is underlined in each of SEQ ID NOS: 126 and 128 below. The conserved glutamic acid (E) is marked in bold at amino acid residue 621 in SEQ ID NO:126 and at amino acid residue 588 in SEQ ID NO:128. The E box motif, in particular the conserved glutamic acid residue, is thought to be important for the formation of oligomeric pilus-like structures of GBS 59. Preferred fragments of GBS 59 include the conserved glutamic acid residue. Preferably, fragments include the E box motif.

SEQ ID NO: 126

MKRINKYFAMFSALLLTLTSLLSVAPAFADEATTNTVTLHKILQTESNLN
KSNFPGTTGLNGKDYKGGAISDLAGYFGEGSKEIEGAFFALALKEDKSGK
VQYVKAKEGNKLTPALINKDGTPEITVNIDEAVSGLTPEGDTGLVFNTKG
LKGEFKIVEVKSKSTYNNNGSLLAASKAVPVNITLPLVNEDGVVADAHVY
PKNTEEKPEIDKNFAKTNDLTALTDVNRLLTAGANYGNYARDKATATAEI
GKVVPYEVKTKIHKGSKYENLVWTDIMSNGLTMGSTVSLKASGTTETFAK
DTDYELSIDARGFTLKFTADGLGKLEKAAKTADIEFTLTYSATVNGQAII
DNPESNDIKLSYGNKPGKDLTELPVTPSKGEVTVAKTWSDGIAPDGVNVV
YTLKDKDKTVASVSLTKTSKGTIDLGNGIKFEVSGNFSGKFTGLENKSYM
ISERVSGYGSAINLENGKVTITNTKDSDNPTPLNPTEPKVETHGKKFVKT
NEQGDRLAGAQFVVKNSAGKYLALKADQSEGQKTLAAKKIALDEAIAAYN
KLSATDQKGEKGITAKELIKTKQADYDAAFIEARTAYEWITDKARAITYT
SNDQGQFEVTGLADGTYNLEETLAPAGFAKLAGNIKFVVNQGSYITGGNI
DYVANSNQKDATRVENKKVTIPQTGGIGTILFTIIGLSIMLGAVVVIMKR
RQSKEA

SEQ ID NO: 128

MKKINKCLTMFSTLLLILTSLFSVAPAFADDATTDTVTLHKIVMPQAAFD
NFTEGTKGKNDSDYVGKQINDLKSYFGSTDAKEIKGAFFVFKNETGTKFI
TENGKEVDTLEAKDAEGGAVLSGLTKDNGFVFNTAKLKGIYQIVELKEKS
NYDNKGSILADSKAVPVKITLPLVNNQGVVKDAHIYPKNTETKPQVDKNF
ADKDLDYTDNRKDKGVVSATVGDKKEYIVGTKILKGSDYKKLVWTDSMTK
GLTFNNNVKVTLDGEDFPVLNYKLVTDDQGFRLALNATGLAAVAAAAKDK
DVEIKITYSATVNGSTTVEIPETNDVKLDYGNNPTEESEPQEGTPANQEI
KVIKDWAVDGTITDANVAVKAIFTLQEKQTDGTWVNVASHEATKPSRFEH
TFTGLDNAKTYRVVERVSGYTPEYVSFKNGVVTIKNNKNSNDPTPINPSE
PKVVTYGRKFVKTNQANTERLAGATFLVKKEGKYLARKAGAATAEAKAAV
KTAKLALDEAVKAYNDLTKEKQEGQEGKTALATVDQKQKAYNDAFVKANY
SYEWVADKKADNVVKLISNAGGQFEITGLDKGTYGLEETQAPAGYATLSG
DVNFEVTATSYSKGATTDIAYDKGSVKKDAQQVQNKKVTIPQTGGIGTIL
FTIIGLSIMLGAVVIMKKRQSEEA

Female mice were immunized with either SAG1407 (SEQ ID NO:126) or B01575 (SEQ ID NO:128) in an active maternal immunization assay. Pups bred from the immunized female mice survived GBS challenge better than control (PBS) treated mice. Results of the active maternal immunization assay using the GBS 59 immunogenic compositions are shown in Table 17, below.

TABLE 17
Active maternal immunization assay for GBS 59

| Challenge | GBS 59 | | PBS | | |
|---|---|---|---|---|---|
| GBS strain | Dead/ | Survival | Dead/ | Survival | |
| (serotype) | treated | (%) | treated | (%) | FACS |
| CJB111 (V)* | 7/20 | 65 | 41/49 | 16 | 493 |
| 18RS21 (II)** | 18/30 | 40 | 39/40 | 2.5 | 380 |

*immunized with BO1575
**immunized with SAG1407

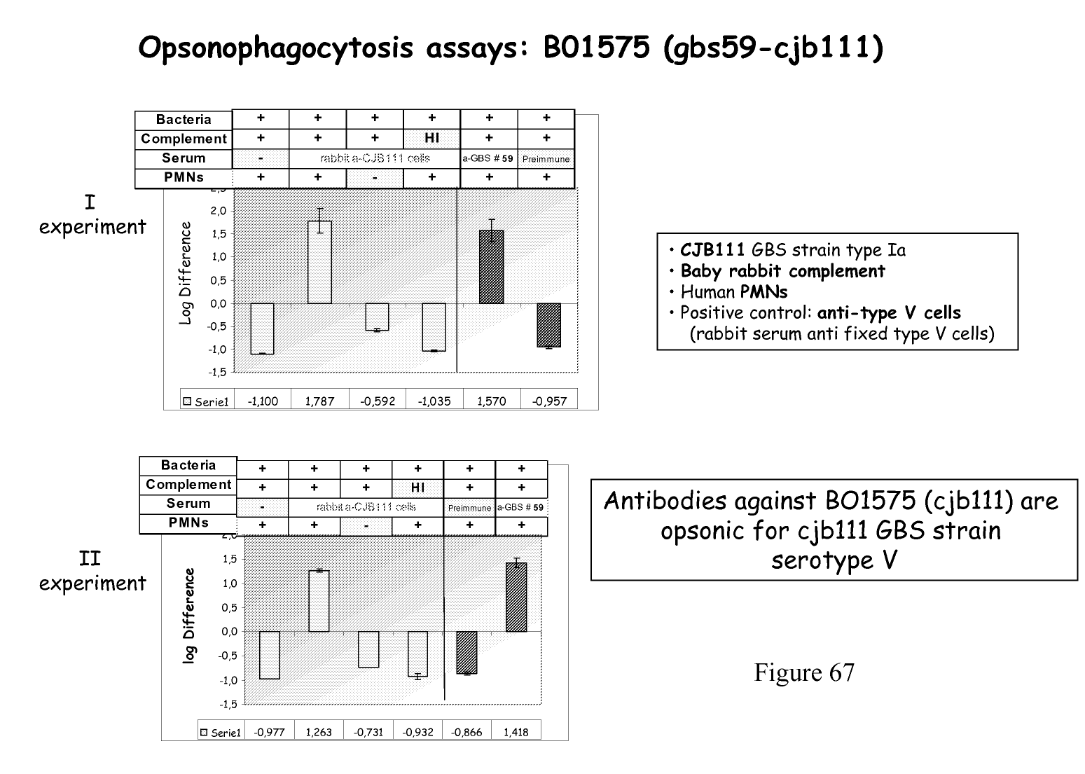

Opsonophagocytosis assays also demonstrated that antibodies against B01575 are opsonic for GBS serotype V, strain CJB111. See FIG. 67.

### GBS 52

Examples of polynucleotide and amino acid sequences for GBS 52 are set forth below. SEQ ID NO:20 and 21 represent GBS 52 sequences from GBS serotype V, strain isolate 2603.

SEQ ID NO: 20

ATGAAACAAACATTAAAACTTATGTTTTCTTTTCTGTTGATGTTAGGGAC
TATGTTTGGAATTAGCCAAACTGTTTTAGCGCAAGAAACTCATCAGTTGA
CGATTGTTCATCTTGAAGCAAGGGATATTGATCGTCCAAATCCACAGTTG
GAGATTGCCCCTAAAGAAGGGACTCCAATTGAAGGAGTACTCTATCAGTT
GTACCAATTAAAATCAACTGAAGATGGCGATTTGTTGGCACATTGGAATT
CCCTAACTATCACAGAATTGAAAAAACAGGCGCAGCAGGTTTTTGAAGCC
ACTACTAATCAACAAGGAAAGGCTACATTTAACCAACTACCAGATGGAAT
TTATTATGGTCTGGCGGTTAAAGCCGGTGAAAAAAATCGTAATGTCTCAG
CTTTCTTGGTTGACTTGTCTGAGGATAAAGTGATTTATCCTAAAATCATC
TGGTCCACAGGTGAGTTGGACTTGCTTAAAGTTGGTGTGGATGGTGATAC
CAAAAAACCACTAGCAGGCGTTGTCTTTGAACTTTATGAAAAGAATGGTA
GGACTCCTATTCGTGTGAAAAATGGGGTGCATTCTCAAGATATTGACGCT
GCAAAACATTTAGAAACAGATTCATCAGGGCATATCAGAATTTCCGGGCT
CATCCATGGGGACTATGTCTTAAAAGAAATCGAGACACAGTCAGGATATC
AGATCGGACAGGCAGAGACTGCTGTGACTATTGAAAAATCAAAAACAGTA
ACAGTAACGATTGAAAATAAAAAAGTTCCGACACCTAAAGTGCCATCTCG
AGGAGGTCTTATTCCCAAAACAGGTGAGCAACAGGCAATGGCACTTGTAA
TTATTGGTGGTATTTTAATTGCTTTAGCCTTACGATTACTATCAAAACAT
CGGAAACATCAAAATAAGGAT

SEQ ID NO: 21

MKQTLKLMFSFLLMLGTMFGTSQTVLAQETHQLTIVHLEARDIDRPNPQL
EIAPKEGTPIEGVLYQLYQLKSTEDGDLLAHWNSLTITELKKQAQQVFEA
TTNQQGKATFNQLPDGIYYGLAVKAGEKNRNVSAFLVDLSEDKVIYPKII
WSTGELDLLKVGVDGDTKKPLAGVVFELYEKNGRTPIRVKNGVHSQDIDA
AKHLETDSSGHIRISGLIHGDYVLKEIETQSGYQIGQAETAVTIEKSKTV
TVTIENKKVPTPKVPSRGGLIPKTGEQQAMALVIIGGILIALALRLLSKH
RKHQNKD

GBS 52 contains an amino acid motif indicative of a cell wall anchor: SEQ ID NO:124 IPKTG (shown in italics in SEQ ID NO:21, above). In some recombinant host cell systems, it may be preferable to remove this motif to facilitate secretion of a recombinant GBS 52 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in GBS 52. The pilin motif sequence is underlined in SEQ ID NO:21, below. Conserved lysine (K) residues are also marked in bold, at amino acid residues 148 and 160. The pilin sequence, in particular the conserved lysine residues, are thought to be important for the formation of oligomeric, pilus-like structures. Preferred fragments of GBS 52 include at least one conserved lysine residue. Preferably, fragments include the pilin sequence.

SEQ ID NO: 21

MKQTLKLMFSFLLMLGTMFGISQTVLAQETHQLTIVHLEARDIDRPNPQL
EIAPKEGTPIEGVLYQLYQLKSTEDGDLLAHWNSLTITELKKQAQQVFEA
TTNQQGKATFNQLPDGIYYGLAVKAGEKNRNVSAFLVDLSEDKVIYPKII
WSTGELDLLKVGVDGDTKKPLAGVVFELYEKNGRTPIRVKNGVHSQDIDA
AKHLETDSSGHIRISGLIHGDYVLKEIETQSGYQIGQAETAVTIEKSKTV
TVTIENKKVPTPKVPSRGGLIPKTGEQQAMALVIIGGILIALALRLLSKH
RKHQNKD

An E box containing a conserved glutamic residue has been identified in GBS 52. The E-box motif is underlined in SEQ ID NO:21, below. The conserved glutamic acid (E), at amino acid residue 226, is marked in bold. The E box motif, in particular the conserved glutamic acid residue, is thought to be important for the formation of oligomeric pilus-like structures of GBS 52. Preferred fragments of GBS 52 include the conserved glutamic acid residue. Preferably, fragments include the E box motif.

SEQ ID NO: 21

MKQTLKLMFSFLLMLGTMFGISQTVLAQETHQLTIVHLEARDIDRPNPQL
EIAPKEGTPIEGVLYQLYQLKSTEDGDLLAHWNSLTITELKKQAQQVFEA
TTNQQGKATFNQLPDGIYYGLAVKAGEKNRNVSAFLVDLSEDKVIYPKII
WSTGELDLLKVGVDGDTKKPLAGVVFELYEKNGRTPIRVKNGVHSQDIDA
AKHLETDSSGHIRISGLIHGDYVLKEIETQSGYQIGQAETAVTIEKSKTV
TVTIENKKVPTPKVPSRGGLIPKTGEQQAMALVIIGGILIALALRLLSKH
RKHQNKD

### SAG0647

Examples of polynucleotide and amino acid sequences for SAG0647 are set forth below. SEQ ID NO:22 and 23 represent SAG0647 sequences from GBS serotype V, strain isolate 2603.

SEQ ID NO: 22

ATGGGACAAAAATCAAAAATATCTCTAGCTACGAATATTCGTATATGGAT
TTTTCGTTTAATTTTCTTAGCGGGTTTCCTTGTTTTGGCATTTCCCATCG
TTAGTCAGGTCATGTACTTTCAAGCCTCTCACGCCAATATTAATGCTTTT
AAAGAAGCTGTTACCAAGATTGACCGGGTGGAGATTAATCGGCGTTTAGA
ACTTGCTTATGCTTATAACGCCAGTATAGCAGGTGCCAAAACTAATGGCG
AATATCCAGCGCTTAAAGACCCCTACTCTGCTGAACAAAAGCAGGCAGGG
GTCGTTGAGTACGCCCGCATGCTTGAAGTCAAAGAACAAATAGGTCATGT
GATTATTCCAAGAATTAATCAGGATATCCCTATTTACGCTGGCTCTGCTG
AAGAAAATCTTCAGAGGGGCGTTGGACATTTAGAGGGGACCAGTCTTCCA
GTCGGTGGTGAGTCAACTCATGCCGTTCTAACTGCCCATCGAGGGCTACC
AACGGCCAAGCTATTTACCAATTTAGACAAGGTAACAGTAGGTGACCGTT
TTTACATTGAACACATCGGCGGAAAGATTGCTTATCAGGTAGACCAAATC
AAAGTTATCGCCCCTGATCAGTTAGAGGATTTGTACGTGATTCAAGGAGA
AGATCACGTCACCCTATTAACTTGCACACCTTATATGATAAATAGTCATC
GCCTCCTCGTTCGAGGCAAGCGAATTCCTTATGTGGAAAAAACAGTGCAG
AAAGATTCAAAGACCTTCAGGCAACAACAATACCTAACCTATGCTATGTG
GGTAGTCGTTGGACTTATCTTGCTGTCGCTTCTCATTTGGTTTAAAAAGA
CGAAACAGAAAAAGCGGAGAAAGAATGAAAAAGCGGCTAGTCAAAATAGT
CACAATAATTCGAAATAA

SEQ ID NO: 23

MGQKSKISLATNIRIWIFRLIFLAGFLVLAFPIVSQVMYFQASHANINAF
KEAVTKIDRVEINRRLELAYAYNASIAGAKTNGEYPALKDPYSAEQKQAG
VVEYARMLEVKEQIGHVIIPRINQDIPIYAGSAEENLQRGVGHLEGTSLP
VGGESTHAVLTAHRGLPTAKLFTNLDKVTVGDRFYIEHIGGKIAYQVDQI
KVIAPDQLEDLYVIQGEDHVTLLTCTPYMINSHRLLVRGKRIPYVEKTVQ
KDSKTFRQQQYLTYANWVVVGLILLSLLIWFKKTKQKKRRKNEKAASQNS
HNNSK

### SAG0648

Examples of polynucleotide and amino acid sequences for SAG0648 are set forth below. SEQ ID NO:24 and 25 represent SAG0648 sequences from GBS serotype V, strain isolate 2603.

SEQ ID NO: 24

ATGGGAAGTCTGATTCTCTTATTTCCGATTGTGAGCCAGGTAAGTTACTA
CCTTGCTTCGCATCAAAATATTAATCAATTTAAGCGGGAAGTCGCTAAGA
TTGATACTAATACGGTTGAACGACGCATCGCTTTAGCTAATGCTTACAAT
GAGACGTTATCAAGGAATCCCTTGCTTATAGACCCTTTTACCAGTAAGCA
AAAAGAAGGTTTGAGAGAGTATGCTCGTATGCTTGAAGTTCATGAGCAAA
TAGGTCATGTGGCAATCCCAAGTATTGGGGTTGATATTCCAATTTATGCT
GGAACATCCGAAACTGTGCTTCAGAAAGGTAGTGGGCATTTGGAGGGAAC
CAGTCTTCCAGTGGGAGGTTTGTCAACCCATTCAGTACTAACTGCCCACC
GTGGCTTGCCAACAGCTAGGCTATTTACCGACTTAAATAAAGTTAAAAAA
GGCCAGATTTTCTATGTGACGAACATCAAGGAAACACTTGCCTACAAAGT
CGTGTCTATCAAAGTTGTGGATCCAACAGCTTTAAGTGAGGTTAAGATTG
TCAATGGTAAGGATTATATAACCTTGCTGACTTGCACACCTTACATGATC
AATAGTCATCGTCTCTTGGTAAAAGGAGAGCGTATTCCTTATGATTCTAC
CGAGGCGGAAAAGCACAAAGAACAAACCGTACAAGATTATCGTTTGTCAC
TAGTGTTGAAGATACTACTAGTATTATTAATTGGACTCTTCATCGTGATA
ATGATGAGAAGATGGATGCAACATCGTCAATAA

SEQ ID NO: 25

MGSLILLFPIVSQVSYYLASHQNINQFKREVAKIDTNTVERRIALANAYN
ETLSRNPLLIDPFTSKQKEGLREYARMLEVHEQIGHVAIPSIGVDIPIYA
GTSETVLQKGSGHLEGTSLPVGGLSTHSVLTAHRGLPTARLFTDLNKVKK
GQIFYVTNIKETLAYKVVSIKVVDPTALSEVKIVNGKDYITLLTCTPYMT
NSHRLLVKGERIPYDSTEAEKHKEQTVQDYRLSLVLKILLVLLIGLFIVI
MMRRWMQHRQ

### GBS 150

Examples of polynucleotide and amino acid sequences for GBS 150 are set forth below. SEQ ID NO:26 and 27 represent GBS 150 sequences from GBS serotype V, strain isolate 2603.

SEQ ID NO: 26

ATGAAAAAGATTAGAAAAAGTTTAGGACTTCTACTATGTTGCTTTTTAGG
ATTGGTACAATTAGCGTTTTTTTCGGTAGCCAGTGTAAATGCTGATACCC
CTAATCAACTAACAATCACACAGATAGGACTTCAGCCAAATACTACAGAG
GAGGGGATTTCTTATCGTTTATGGACTGTGACTGACAACTTAAAAGTTGA
TTTATTGAGCCAAATGACAGATAGCGAATTGAACCAGAAGTATAAGAGTA
TCTTGACTTCTCCTACTGATACTAATGGTCAGACAAAGATAGCACTCCCA
AATGGTTCGTACTTTGGTCGTGCTTATAAAGCTGATCAAAGCGTTTCAAC
AATAGTACCTTTTTATATTGAATTACCAGATGATAAGTTATCAAATCAAT
TACAGATAAATCCTAAGCGAAAAGTTGAAACAGGCCGATTAAAACTTATT
AAATATACAAAAGAAGGAAAGATAAAGAAAAGGCTATCCGGAGTAATATT
TGTATTATACGATAACCAGAATCAGCCAGTTCGCTTTAAAAATGGACGAT
TTACGACCGATCAAGATGGGATTACTTCATTAGTAACTGATGATAAGGGA
GAAATTGAGGTTGAAGGTTTATTACCTGGTAAGTATATTTTTCGAGAAGC
AAAAGCACTAACTGGTTACCGTATATCTATGAAGGATGCTGTAGTTGCTG
TAGTTGCTAATAAAACACAGGAAGTAGAGGTAGAAAACGAAAAAGAAACT
CCTCCACCAACAAATCCTAAACCATCACAACCGCTTTTTCCACAATCATT
TCTTCCTAAAACAGGAATGATTATTGGTGGAGGACTGACAATTCTTGGTT
GTATTATTTTGGGAATTTTGTTTATCTTTTTAAGAAAAACTAAAAATAGC
AAATCTGAAAGAAACGATACAGTA

SEQ ID NO: 27

MKKIRKSLGLLLCCFLGLVQLAFFSVASVNADTPNQLTITQIGLQPNTTE
EGISYRLWTVTDNLKVDLLSQMTDSELNQKYKSILTSPTDTNGQTKIALP
NGSYFGRAYKADQSVSTIVPFYIELPDDKLSNQLQINPKRKVETGRLKLI
KYTKEGKIKKRLSGVIFVLYDNQNQPVRFKNGRFTTDQDGITSLVTDDKG
EIEVEGLLPGKYIFREAKALTGYRISMKDAVVAVVANKTQEVEVENEKET
PPPTNPKPSQPLFPQSFLPKTGMIIGGGLTILGCIILGILFIFLRKTKNS
KSERNDTV

GBS 150 contains an amino acid motif indicative of a cell wall anchor: SEQ ID NO:130 LPKTG (shown in italics in SEQ ID NO:27 above). In some recombinant host cell systems, it may be preferable to remove this motif to facilitate secretion of a recombinant GBS 150 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

As discussed above, a pilin motif, containing a conserved lysine (K) residue has been identified in GBS 150. The pilin motif sequence is underlined in SEQ ID NO:27, below. Conserved lysine (K) residues are marked in bold, at amino acid residues 139 and 148. The pilin sequence, in particular the conserved lysine residues, are thought to be important for the formation of oligomeric, pilus-like structures of GBS 150. Preferred fragments of GBS 150 include a conserved lysine residue. Preferably, fragments include the pilin sequence.

SEQ ID NO: 27

MKKIRKSLGLLLCCFLGLVQLAFFSVASVNADTPNQLTITQIGLQPNTTE
EGISYRLWTVTDNLKVDLLSQMTDSELNQKYKSILTSPTDTNGQTKIALP
NGSYFGRAYKADQSVSTIVPFYIELPDDKLSNQLQINPKRKVETGRLKLI
KYTKEGKIKKRLSGVIFVLYDNQNQPVRFKNGRFTTDQDGITSLVTDDKG
EIEVEGLLPGKYIFREAKALTGYRISMKDAVVAVVANKTQEVEVENEKET
PPPTNPKPSQPLFPQSFLPKTGMIIGGGLTILGCIILGILFIFLRKTKNS
KSERNDTV

An E box containing a conserved glutamic residue has also been identified in GBS 150. The E box motif is underlined in SEQ ID NO:27 below. The conserved glutamic acid (E), at amino acid residue 216, is marked in bold. The E box motif, in particular the conserved glutamic acid residue, is thought to be important for the formation of oligomeric pilus-like structures of GBS 150. Preferred fragments of GBS 150 include the conserved glutamic acid residue. Preferably, fragments include the E box motif.

SEQ ID NO: 27

MKKIRKSLGLLLCCFLGLVQLAFFSVASVNADTPNQLTITQIGLQPNTTE
EGISYRLWTVTDNLKVDLLSQMTDSELNQKYKSILTSPTDTNGQTKIALP
NGSYFGRAYKADQSVSTIVPFYIELPDDKLSNQLQINPKRKVETGRLKLI
KYTKEGKIKKRLSGVIFVLYDNQNQPVRFKNGRFTTDQDGITSLVTDDKG
EIEVEGLLPGKYIFREAKALTGYRISMKDAVVAVVANKTQEVEVENEKET
PPPTNPKPSQPLFPQSFLPKTGMIIGGGLTILGCIILGILFIFLRKTKNS
KSERNDTV

### SAG 1405

Examples of polynucleotide and amino acid sequences for SAG1405 are set forth below. SEQ ID NO:28 and 29 represent SAG1405 sequences from GBS serotype V, strain isolate 2603.

SEQ ID NO: 28

ATGGGAGGAAAATTTCAGAAAAACCTTAAGAAATCGGTCGTTTTAAATCG
ATGGATGAATGTAGGCTTGATACTATTGTTCTTAGTTGGTCTTTTGATAA
CCTCATATCCTTTTATTTCAAATTGGTACTATAATATTAAAGCTAATAAT
CAAGTAACTAACTTTGATAATCAAACCCAAAAATTAAATACTAAAGAGAT
TAATAGACGATTTGAGTTAGCAAAAGCTTATAATAGAACACTGGACCCAA
GCCGCCTATCAGATCCCTATACTGAAAAAGAAAAAAAAGGTATTGCTGAA
TACGCCCACATGCTTGAGATTGCTGAAATGATTGGATATATTGATATACC
GTCTATCAAGCAAAAATTACCTATCTATGCGGGGACTACCAGTAGTGTTC
TTGAAAAAGGAGCAGGACACCTTGAAGGAACCTCCTTGCCAATTGGTGGA
AAAAGTTCACATACTGTTATCACAGCTCATCGCGGCTTACCTAAAGCTAA
GTTATTTACAGATTTAGATAAACTTAAAAAAGGAAAAATTTTTTATATTC
ATAATATCAAAGAAGTTTTAGCCTATAAGGTTGATCAAATAAGTGTTGTA
AAGCCAGATAATTTTTCTAAATTATTGGTTGTTAAAGGTAAGGATTATGC
GACTTTGCTAACATGTACACCTTATTCGATTAATTCACATCGTTTACTAG
TTAGAGGGCATCGAATCAAGTATGTACCTCCTGTTAAAGAAAAGAACTAT
TTAATGAAAGAATTGCAAACACACTATAAACTTTATTTCCTCTTATCAAT
CCTAGTTATTCTTATATTAGTCGCTTTACTATTATATTTAAAACGAAAAT
TTAAAGAGAGAAAGAGAAAGGGAAATCAAAAATGA

SEQ ID NO: 29

MGGKFQKNLKKSVVLNRWMNVGLILLFLVGLLITSYPFISNWYYNTKANN
QVTNFDNQTQKLNTKEINRRFELAKAYNRTLDPSRLSDPYTEKEKKGIAE
YAHMLEIAEMIGYIDIPSIKQKLPIYAGTTSSVLEKGAGHLEGTSLPIGG
KSSHTVITAHRGLPKAKLFTDLDKLKKGKTFYTHNTKEVLAYKVDQISVV
KPDNFSKLLVVKGKDYATLLTCTPYSINSHRLLVRGHRIKYVPPVKEKNY
LMKELQTHYKLYFLLSILVILILVALLLYLKRKFKERKRKGNQK

### SAG1406

Examples of polynucleotide and amino acid sequences for SAG1405 are set forth below. SEQ ID NO:30 and 31 represent SAG1405 sequences from GBS serotype V, strain isolate 2603.

SEQ ID NO: 30

GTGAAGACTAAAAAAATCATCAAAAAAACAAAAAAAAAGAAGAAGTCAAA
TCTTCCTTTTATCATTCTTTTTCTAATAGGTCTATCTATTTTATTGTATC
CAGTGGTATCACGTTTTTACTATACGATAGAATCTAATAATCAAACACAG
GATTTTGAGAGAGCTGCTAAAAACTTAGTCAGAAAGAAATCAATCGACGT
ATGGCTCTAGCACAAGCTTATAATGATTCTTTAAATAATGTCCATCTTGA
AGATCCTTATGAGAAAAAACGAATTCAAAAGGGGGTAGCAGAGTACGCCC
GTATGTTAGAGGTAAGTGAAAAAATCGGAACAATTTCAGTTCCTAAGATA
GGTCAAAAACTCCCTATATTTGCAGGTTCAAGTCAAGAAGTTCTATCTAA
AGGAGCAGGGCATTTAGAAGGTACCTCTCTTCCAATTGGGGGCAATAGTA
CACATACTGTTATAACAGCGCATTCAGGAATTCCAGATAAAGAACTCTTT
TCTAACCTTAAAAAGTTAAAAAAAGGAGATAAGTTTTATATTCAAAACAT
AAAAGAAACGATAGCATATCAAGTAGATCAGATAAAAGTCGTTACACCCG
ATAACTTTTCAGATTTGTTGGTTGTTCCTGGACATGATTATGCAACCTTA
TTGACTTGCACCCCGATTATGATCAATACACACAGACTTTTAGTAAGGGG
ACATCGTATCCCTTATAAAGGTCCTATTGATGAAAAATTAATAAAAGACG
GTCATTTAAACACGATTTATAGATATCTATTCTATATATCTTTAGTTATT
ATTGCTTGGTTACTTTGGTTAATAAAACGTCAACGTCAAAAAAATCGTTT
AGCAAGTGTTAGAAAAGGAATTGAATCATAA

SEQ ID NO: 31

MKTKKIIKKTKKKKKSNLPFIILFLIGLSILLYPVVSRFYYTIESNNQTQ
DFEPAAKKLSQKETNRRMALAQAYNDSLNNVHLEDPYEKKRIQKGVAEYA
RMLEVSEKIGTISVPKIGQKLPIFAGSSQEVLSKGAGHLEGTSLPIGGNS
THTVITAHSGIPDKELFSNLKKLKKGDKFYIQNIKETIAYQVDQIKVVTP
DNFSDLLVVPGHDYATLLTCTPIMINTHRLLVRGHRIPYKGPIDEKLIKD
GHLNTIYRYLFYTSLVIIAWLLWLIKRQRQKNRLASVRKGIES

01520

An example of an amino acid sequence for 01520 is set forth below. SEQ ID NO:32 represents a 01520 sequence from GBS serotype III, strain isolate COH1.

SEQ ID NO: 32

MIRRYSANFLAILGIILVSSGIYWGWYNINQAHQADLTSQHIVKVLDKSI
THQVKGSENGELPVKKLDKTDYLGTLDIPNLKLHLPVAANYSFEQLSKTP
TRYYGSYLTNNMVICAHNFPYHFDALKNVDMGTDVYFTTTTGQIYHYKIS
NREIIEPTAIEKVYKTATSDNDWDLSLFTCTKAGVARVLVRCQLIDVKN

01521

An example of an amino acid sequence for 01521 is set forth below. SEQ ID NO:33 represents a 01521 sequence from GBS serotype III, strain isolate COH1.

SEQ ID NO: 33

MIYKKILKITLLLLFSLSTQLVSADTNDQMKTGSITIQNKYNNQGIAGGN
LLVYQVAQAKDVDGNQVFTLTTPFQOIGIKDDDLTQVNLDSNQAKYVNLL
TKAVHKTQPLQTFDNLPAEGIVANNLPQGIYLFIQTKTAQGYELMSPFIL
SIPKDGKYDITAFEKMSPLNAKPKKEETITPTVTHQTKGKLPFTGQVWWP
IPILIMSGLLCLIIALKWRRRRD

01521 contains an amino acid motif indicative of a cell wall anchor: SEQ ID NO:132 LPFTG (shown in italics in SEQ ID NO:33 above). In some recombinant host cell systems, it may be preferable to remove this motif to facilitate secretion of a recombinant 01521 protein from the host cell. Alternatively, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Two pilin motifs, containing conserved lysine (K) residues have been identified in 01521. The pilin motif sequences are underlined in SEQ ID NO:33, below. Conserved lysine (K) residues are marked in bold, at amino acid residues 154 and 165 and at amino acid residues 174 and 188. The pilin sequences, in particular the conserved lysine residues, are thought to be important for the formation of oligomeric, pilus-like structures of 01521. Preferred fragments of 01521 include at least one conserved lysine residue. Preferably, fragments include at least one pilin sequence.

SEQ ID NO: 33

MIYKKILKITLLLLFSLSTQLVSADTNDQMKTGSITIQNKYNNQGIAGGN
LLVYQVAQAKDVDGNQVFTLTTPFQOIGIKDDDLTQVNLDSNQAKYVNLL
TKAVHKTQPLQTFDNLPAEGIVANNLPQGIYLFIQTKTAQGYELMSPFIL
SIPKDGKYDITAFEKMSPLNAKPKKEETITPTVTHQTKGKLPFTGQVWWP
IPILIMSGLLCLIIALKWRRRRD

An E box containing a conserved glutamic residue has also been identified in 01521. The E box motif is underlined in SEQ ID NO:33 below. The conserved glutamic acid (E), at amino acid residue 177, is marked in bold. The E box motif, in particular the conserved glutamic acid residue, is thought to be important for the formation of oligomeric pilus-like structures of 01521. Preferred fragments of 01521 include the conserved glutamic acid residue. Preferably, fragments include the E box motif.

SEQ ID NO: 33

MIYKKILKITLLLLFSLSTQLVSADTNDQMKTGSITIQNKYNNQGIAGGN
LLVYQVAQAKDVDGNQVFTLTTPFQOIGIKDDDLTQVNLDSNQAKYVNLL
TKAVHKTQPLQTFDNLPAEGIVANNLPQGIYLFIQTKTAQGYELMSPFIL
SIPKDGKYDITAFEKMSPLNAKPKKEETITPTVTHQTKGKLPFTGQVWWP
IPILIMSGLLCLIIALKWRRRRD

01522

An example of an amino acid sequence for 01522 is set forth below. SEQ ID NO:34 represents a 01522 sequence from GBS serotype III, strain isolate COH1.

SEQ ID NO: 34

MAYPSLANYWNSFHQSRAIMDYQDRVTHMDENDYKKIINRAKEYNKQFKT
SGMKWHMTSQERLDYNSQLAIDKTGNMGYISIPKINIKLPLYHGTSEKVL
QTSIGHLEGSSLPIGGDSTHSILSGHRGLPSSRLFSDLDKLKVGDHWTVS
ILNETYTYQVDQIRTVKPDDLRDLQIVKGKDYQTLVTCTPYGVNTHRLLV
RGHRVPNDNGNALVVAEAIQIEPIYIAPFIAIFLTLILLLISLEVTRPAR
QRKKILKQAMRKEENNDL

01523

An example of an amino acid sequence for 01523 is set forth below. SEQ ID NO:35 represents a 01523 sequence from GBS serotype III, strain isolate COH1.

SEQ ID NO: 35

MKKKMIQSLLVASLAFGMAVSPVTPIAFAAETGTITVQDTQKGATYKAYK
VFDAEIDNANVSDSNKDGASYLIPQGKEAEYKASTDFNSLFTTTTNGGRT
YVTKKDTASANEIATWAKSISANTTPVSTVTESNNDGTEVINVSQYGYYY
VSSTVNNGAVIMVTSVTPNATIHEKNTDATWGDGGGKTVDQKTYSVGDTV
KYTITYKNAVNYHGTEKVYQYVIKDTMPSASVVDLNEGSYEVTITDGSGN
ITTLTQGSEKATGKYNLLEENNNFTITIPWAATNTPTGNTQNGANDDFFY
KGINTITVTYTGVLKSGAKPGSADLPENTNIATINPNTSNDDPGQKVTVR
DGQITIKKIDGSTKASLQGAIFVLKNATGQFLNFNDTNNVEWGTEANATE
YTTGADGIITITGLKEGTYYLVEKKAPLGYNLLDNSQKVILGDGATDTTN
SDNLLVNPTVENNKGTELPSTGGIGTTIFYIIGAILVIGAGIVLVARRRL
RS

01523 contains an amino acid motif indicative of a cell wall anchor: SEQ ID NO:131 LPSTG (shown in italics in SEQ ID NO:35 above). In some recombinant host cell systems, it may be preferable to remove this motif to facilitate secretion of a recombinant 01523 protein from the host cell. Alternatively, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

An E box containing a conserved glutamic residue has also been identified in 01523. The E box motif is underlined in SEQ ID NO:35 below. The conserved glutamic acid (E), at amino acid residue 423, is marked in bold. The E box motif, in particular the conserved glutamic acid residue, is thought to be important for the formation of oligomeric pilus-like structures of 01523. Preferred fragments of 01523 include the conserved glutamic acid residue. Preferably, fragments include the E box motif.

SEQ ID NO: 35

MKKKMIQSLLVASLAFGMAVSPVTPIAFAAETGTITVQDTQKGATYKAYK
VFDAEIDNANVSDSNKDGASYLIPQGKEAEYKASTDFNSLFTTTTNGGRT
YVTKKDTASANEIATWAKSISANTTPVSTVTESNNDGTEVINVSQYGYYY
VSSTVNNGAVIMVTSVTPNATIHEKNTDATWGDGGGKTVDQKTYSVGDTV
KYTITYKNAVNYHGTEKVYQYVIKDTMPSASVVDLNEGSYEVTITDGSGN
ITTLTQGSEKATGKYNLLEENNNFTITIPWAATNTPTGNTQNGANDDFFY
KGINTITVTYTGVLKSGAKPGSADLPENTNIATINPNTSNDDPGQKVTVR
DGQITIKKIDGSTKASLQGAIFVLKNATGQFLNFNDTNNVEWGTEANATE
YTTGADGIITITGLKEGTYYLVEKKAPLGYNLLDNSQKVILGDGATDTTN
SDNLLVNPTVENNKGTELPSTGGIGTTIFYIIGAILVIGAGIVLVARRRL
RS

01524

An example of an amino acid sequence for 01524 is set forth below. SEQ ID NO:36 represents a 01524 sequence from GBS serotype III, strain isolate COH1.

SEQ ID NO: 36

MLKKCQTFIIESLKKKKHPKEWKIIMWSLMILTTFLTTYFLILPAITVEE
TKTDDVGITLENKNSSQVTSSTSSSQSSVEQSKPQTPASSVTETSSSEEA
AYREEPLMFRGADYTVTVTLTKEAKIPKNADLKVTELKDNSATFKDYKKK
ALTEVAKQDSEIKNFKLYDITIESNGKEAEPQAPVKVEVNYDKPLEASDE
NLKVVHFKDDGQTEVLKSKDTAETKNTSSDVAFKTDSFSIYAIVQEDNTE
VPRLTYHFQNNDGTDYDFLTASGMQVHHQIIKDGESLGEVGIPTIKAGEH
FNGWYTYDPTTGKYGDPVKFGEPITVTETKEICVRPFMSKVATVTLYDDS
AGKSILERYQVPLDSSGNGTADLSSFKVSPPTSTLLFVGWSKTQNGAPLS
ESETQALPVSSDISLYPVFKESYGVEFNTGDLSTGVTYIAPRRVLTGQPA
STIKPNDPTRPGYTFAGWYTAASGGAAFDFNQVLTKDTTLYAHWSPAQTT
YTINYWQQSATDNKNATDAQKTYEYAGQVTRSGLSLSNQTLTQQDINDKL
PTGFKVNNTRTETSVMIKDDGSSVVNVYYDRKLITIKFAKYGGYSLPEYY
YSYNWSSDADTYTGLYGTTLAANGYQWKTGAWGYLANVGNNQVGTYGMSY
LGEFILPNDTVDSDVIKLFPKGNIVQTYRFFKQGLDGTYSLADTGGGAGA
DEFTFTEKYLGFNVKYYQRLYPDNYLFDQYASQTSAGVKVPISDEYYDRY
GAYHKDYLNLVVWYERNSYKIKYLDPLDNTELPNFPVKDVLYEQNLSSYA
PDTTTVQPKPSRPGYVWDGKWYKDQAQTQVFDFNTTMPPHDVKVYAGWQK
VTYRVNIDPNGGRLSKTDDTYLDLHYGDRIPDYTDITRDYIQDPSGTYYY
KYDSRDKDPDSTKDAYYTTDTSLSNVDTTTKYKYVKDAYKLVGWYYVNPD
GSIRPYNFSGAVTQDTNLRAIWRKAGDYHIIYSNDAVGTDGKPALDASGQ
QLQTSNEPTDPDSYDDGSHSALLRRPTMPDGYRFRGWWYNGKIYNPYDSI
DIDAHLADANKNITIKPVIIPVGDIKLEDTSIKYNGNGGTRVENGNVVTQ
VETPRMELNSTTTIPENQYFTRTGYNLIGWHHDKDLADTGRVEFTAGQSI
GIDNNPDATNTLYAVWQPKEYTVRVSKTVVGLDEDKTKDFLFNPSETLQQ
ENFPLRDGQTKEFKVPYGTSISTDEQAYDEFKVSESITEKNLATGEADKT
YDATGLQSLTVSGDVDISFTNTRIKQKVRLQKVNVENDNNFLAGAVFDIY
ESDANGNKASHPMYSGLVTNDKGLLLVDANNYLSLPVGKYYLTETKAPPG
YLLPKNDISVLVISTGVTFEQNGNNATPIKENLVDGSTVYTFKITNSKGT
ELPSTGGTGTHIYTLVGLALALPSGLILYYRKKI

01524 contains an amino acid motif indicative of a cell wall anchor: SEQ ID NO:131 LPSTG (shown in italics in SEQ ID NO:36 above). In some recombinant host cell systems, it may be preferable to remove this motif to facilitate secretion of a recombinant 01524 protein from the host cell. Alternatively, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Three pilin motifs, containing conserved lysine (K) residues have been identified in 01524. The pilin motif sequences are underlined in SEQ ID NO:36, below. Conserved lysine (K) residues are marked in bold, at amino acid residues 128 and 138, amino acid residues 671 and 682, and amino acid residues 809 and 820. The pilin sequences, in particular the conserved lysine residues, are thought to be important for the formation of oligomeric, pilus-like structures of 01524. Preferred fragments of 01524 include at least one conserved lysine residue. Preferably, fragments include at least one pilin sequence.

SEQ ID NO: 36

MLKKCQTFIIESLKKKKHPKEWKIIMWSLMILTTFLTTYFLILPAITVEE
TKTDDVGITLENKNSSQVTSSTSSSQSSVEQSKPQTPASSVTETSSSEEA
AYREEPLMFRGADYTVTVTLTKEAKIPKNADLKVTELKDNSATFKDYKKK
ALTEVAKQDSEIKNFKLYDITIESNGKEAEPQAPVKVEVNYDKPLEASDE
NLKVVHFKDDGQTEVLKSKDTAETKNTSSDVAFKTDSFSIYAIVQEDNTE
VPRLTYHFQNNDGTDYDFLTASGMQVHHQIIKDGESLGEVGIPTIKAGEH
FNGWYTYDPTTGKYGDPVKFGEPITVTETKEICVRPFMSKVATVTLYDDS
AGKSILERYQVPLDSSGNGTADLSSFKVSPPTSTLLFVGWSKTQNGAPLS
ESEIQALPVSSDISLYPVFKESYGVEFNTGDLSTGVTYIAPRRVLTGQPA
STIKPNDPTRPGYTFAGWYTAASGGAAFDFNQVLTKDTTLYAHWSPAQTT
YTINYWQQSATDNKNATDAQKTYEYAGQVTRSGLSLSNQTLTQQDINDKL
PTGFKVNNTRTETSVMIKDDGSSVVNVYYDRKLITIKFAKYGGYSLPEYY
YSYNWSSDADTYTGLYGTTLAANGYQWKTGAWGYLANVGNNQVGTYGMSY
LGEFILPNDTVDSDVIKLFPKGNIVQTYRFFKQGLDGTYSLADTGGGAGA
DEFTFTEKYLGFNVKYYQRLYPDNYLFDQYASQTSAGVKVPISDEYYDRY
GAYHKDYLNLVVWYERNSYKIKYLDPLDNTELPNFPVKDVLYEQNLSSYA
PDTTTVQPKPSRPGYVWDGKWYKDQAQTQVFDFNTTMPPHDVKVYAGWQK
VTYRVNIDPNGGRLSKTDDTYLDLHYGDRIPDYTDITRDYIQDPSGTYYY
KYDSRDKDPDSTKDAYYTTDTSLSNVDTTTKYKYVKDAYKLVGWYYVNPD
GSIRPYNFSGAVTQDINLRAIWRKAGDYHIIYSNDAVGTDGKPALDASGQ
QLQTSNEPTDPDSYDDGSHSALLRRPTMPDGYRFRGWWYNGKIYNPYDSI
DIDAHLADANKNITIKPVIIPVGDIKLEDTSIKYNGNGGTRVENGNVVTQ
VETPRMELNSTTTIPENQYFTRTGYNLIGWHHDKDLADTGRVEFTAGQSI
GIDNNPDATNTLYAVWQPKEYTVRVSKTVVGLDEDKTKDFLFNPSETLQQ
ENFPLRDGQTKEFKVPYGTSISIDEQAYDEFKVSESITEKNLATGEADKT
YDATGLQSLTVSGDVDISFTNTRIKQKVRLQKVNVENDNNFLAGAVFDIY
ESDANGNKASHPMYSGLVTNDKGLLLVDANNYLSLPVGKYYLTETKAPPG
YLLPKNDISVLVISTGVTFEQNGNNATPIKENLVDGSTVYTFKITNSKGT
ELPSTGGIGTHIYILVGLALALPSGLILYYRKKI

An E box containing a conserved glutamic residue has also been identified in 01524. The E box motif is underlined in SEQ ID NO:36 below. The conserved glutamic acid (E), at amino acid residue 1344, is marked in bold. The E box motif, in particular the conserved glutamic acid residue, is thought to be important for the formation of oligomeric pilus-like structures of 01524. Preferred fragments of 01524 include the conserved glutamic acid residue. Preferably, fragments include the E box motif.

SEQ ID NO: 36

MLKKCQTFIIESLKKKKHPKEWKIIMWSLMILTTFLTTYFLILPAITVEE
TKTDDVGITLENKNSSQVTSSTSSSQSSVEQSKPQTPASSVTETSSSEEA
AYREEPLMFRGADYTVTVTLTKEAKIPKNADLKVTELKDNSATFKDYKKK
ALTEVAKQDSEIKNFKLYDITIESNGKEAEPQAPVKVEVNYDKPLEASDE
NLKVVHFKDDGQTEVLKSKDTAETKNTSSDVAFKTDSFSIYAIVQEDNTE
VPRLTYHFQNNDGTDYDFLTASGMQVHHQIIKDGESLGEVGIPTIKAGEH
FNGWYTYDPTTGKYGDPVKFGEPITVTETKEICVRPFMSKVATVTLYDDS
AGKSILERYQVPLDSSGNGTADLSSFKVSPPTSTLLFVGWSKTQNGAPLS
ESEIQALPVSSDISLYPVFKESYGVEFNTGDLSTGVTYIAPRRVLTGQPA
STIKPNDPTRPGYTFAGWYTAASGGAAFDFNQVLTKDTTLYAHWSPAQTT
YTINYWQQSATDNKNATDAQKTYEYAGQVTRSGLSLSNQTLTQQDINDKL
PTGFKVNNTRTETSVMIKDDGSSVVNVYYDRKLITIKFAKYGGYSLPEYY
YSYNWSSDADTYTGLYGTTLAANGYQWKTGAWGYLANVGNNQVGTYGMSY
LGEFILPNDTVDSDVIKLFPKGNIVQTYRFFKQGLDGTYSLADTGGGAGA
DEFTFTEKYLGFNVKYYQRLYPDNYLFDQYASQTSAGVKVPISDEYYDRY
GAYHKDYLNLVVWYERNSYKIKYLDPLDNTELPNFPVKDVLYEQNLSSYA
PDTTTVQPKPSRPGYVWDGKWYKDQAQTQVFDFNTTMPPHDVKVYAGWQK
VTYRVNIDPNGGRLSKTDDTYLDLHYGDRIPDYTDITRDYIQDPSGTYYY
KYDSRDKDPDSTKDAYYTTDTSLSNVDTTTKYKYVKDAYKLVGWYYVNPD
GSIRPYNFSGAVTQDINLRAIWRKAGDYHIIYSNDAVGTDGKPALDASGQ
QLQTSNEPTDPDSYDDGSHSALLRRPTMPDGYRFRGWWYNGKIYNPYDSI
DIDAHLADANKNITIKPVIIPVGDIKLEDTSIKYNGNGGTRVENGNVVTQ
VETPRMELNSTTTIPENQYFTRTGYNLIGWHHDKDLADTGRVEFTAGQSI
GIDNNPDATNTLYAVWQPKEYTVRVSKTVVGLDEDKTKDFLFNPSETLQQ
ENFPLRDGQTKEFKVPYGTSISIDEQAYDEFKVSESITEKNLATGEADKT
YDATGLQSLTVSGDVDISFTNTRIKQKVRLQKVNVENDNNFLAGAVFDIY
ESDANGNKASHPMYSGLVTNDKGLLLVDANNYLSLPVGKYYLTETKAPPG
YLLPKNDISVLVISTGVTFEQNGNNATPIKENLVDGSTVYTFKITNSKGT
ELPSTGGIGTHIYILVGLALALPSGLILYYRKKI

01525

An example of an amino acid sequence for 01525 is set forth below. SEQ ID NO:37 represents a 01525 sequence from GBS serotype III, strain isolate COH1.

SEQ ID NO: 37

MKRQISSDKLSQELDRVTYQKRFWSVIKNTIYILMAVASIAILIAVLWLP
VLRIYGHSMNKTLSAGDVVFTVKGSNFKTGDVVAFYYNNKVLVKRVIAES
GDWVNIDSQGDVYVNQHKLKEPYVIHKALGNSNIKYPYQVPDKKIFVLGD
NRKTSIDSRSTSVGDVSEEQIVGKISFRIWPLGKISSIN

### GBS 322

GBS 322 refers to a surface immunogenic protein, also referred to as “sip”. Nucleotide and amino acid sequences of GBS 322 sequenced from serotype V isolated strain 2603 V/R are set forth in Ref. 3 as SEQ ID 8539 and SEQ ID 8540. These sequences are set forth below as SEQ ID NOS 38 and 39:

SEQ ID NO. 38

ATGAATAAAAAGGTACTATTGACATCGACAATGGCAGCTTCGCTATTATC
AGTCGCAAGTGTTCAAGCACAAGAAACAGATACGACGTGGACAGCACGTA
CTGTTTCAGAGGTAAAGGCTGATTTGGTAAAGCAAGACAATAAATCATCA
TATACTGTGAAATATGGTGATACACTAAGCGTTATTTCAGAAGCAATGTC
AATTGATATGAATGTCTTAGCAAAAATAAATAACATTGCAGATATCAATC
TTATTTATCCTGAGACAACACTGACAGTAACTTACGATCAGAAGAGTCAT
ACTGCCACTTCAATGAAAATAGAAACACCAGCAACAAATGCTGCTGGTCA
AACAACAGCTACTGTGGATTTGAAAACCAATCAAGTTTCTGTTGCAGACC
AAAAAGTTTCTCTCAATACAATTTCGGAAGGTATGACACCAGAAGCAGCA
ACAACGATTGTTTCGCCAATGAAGACATATTCTTCTGCGCCAGCTTTGAA
ATCAAAAGAAGTATTAGCACAAGAGCAAGCTGTTAGTCAAGCAGCAGCTA
ATGAACAGGTATCACCAGCTCCTGTGAAGTCGATTACTTCAGAAGTTCCA
GCAGCTAAAGAGGAAGTTAAACCAACTCAGACGTCAGTCAGTCAGTCAAC
AACAGTATCACCAGCTTCTGTTGCCGCTGAAACACCAGCTCCAGTAGCTA
AAGTAGCACCGGTAAGAACTGTAGCAGCCCCTAGAGTGGCAAGTGTTAAA
GTAGTCACTCCTAAAGTAGAAACTGGTGCATCACCAGAGCATGTATCAGC
TCCAGCAGTTCCTGTGACTACGACTTCACCAGCTACAGACAGTAAGTTAC
AAGCGACTGAAGTTAAGAGCGTTCCGGTAGCACAAAAAGCTCCAACAGCA
ACACCGGTAGCACAACCAGCTTCAACAACAAATGCAGTAGCTGCACATCC
TGAAAATGCAGGGCTCCAACCTCATGTTGCAGCTTATAAAGAAAAAGTAG
CGTCAACTTATGGAGTTAATGAATTCAGTACATACCGTGCGGGAGATCCA
GGTGATCATGGTAAAGGTTTAGCAGTTGACTTTATTGTAGGTACTAATCA
AGCACTTGGTAATAAAGTTGCACAGTACTCTACACAAAATATGGCAGCAA
ATAACATTTCATATGTTATCTGGCAACAAAAGTTTTACTCAAATACAAAC
AGTATTTATGGACCTGCTAATACTTGGAATGCAATGCCAGATCGTGGTGG
CGTTACTGCCAACCACTATGACCACGTTCACGTATCATTTAACAAATAAT
ATAAAAAAGGAAGCTATTTGGCTTCTTTTTTATATGCCTTGAATAGACTT
TCAAGGTTCTTATATAATTTTTATTA

SEQ ID NO. 39

MNKKVLLTSTMAASLLSVASVQAQETDTTWTARTVSEVKADLVKQDNKSS
YTVKYGDTLSVISEAMSIDMNVLAKINNIADINLIYPETTLTVTYDQKSH
TATSMKIETPATNAAGQTTATVDLKTNQVSVADQKVSLNTISEGMTPEAA
TTIVSPMKTYSSAPALKSKEVLAQEQAVSQAAANEQVSPAPVKSITSEVP
AAKEEVKPTQTSVSQSTTVSPASVAAETPAPVAKVAPVRTVAAPRVASVK
VVTPKVETGASPEHVSAPAVPVTTTSPATDSKLQATEVKSVPVAQKAPTA
TPVAQPASTTNAVAAHPENAGLQPHVAAYKEKVASTYGVNEFSTYRAGDP
GDHGKGLAVDFIVGTNQALGNKVAQYSTQNMAANNISYVIWQQKFYSNTN
SIYGPANTWNAMPDRGGVTANHYDHVHVSFNK

GBS 322 contains an N-terminal leader or signal sequence region which is indicated by the underlined sequence near the beginning of SEQ ID NO:39. In one embodiment, one or more amino acids from the leader or signal sequence region of GBS 322 are removed. An example of such a GBS 322 fragment is set forth below as SEQ ID NO:40.

SEQ ID NO: 40

DLVKQDNKSSYTVKYGDTLSVISEAMSIDMNVLAKINNIADINLIYPETT
LTVTYDQKSHTATSMKIETPATNAAGQTTATVDLKTNQVSVADQKVSLNT
ISEGMTPEAATTIVSPMKTYSSAPALKSKEVLAQEQAVSQAAANEQVSPA
PVKSITSEVPAAKEEVKPTQTSVSQSTTVSPASVAAETPAPVAKVAPVRT
VAAPRVASVKVVTPKVETGASPEHVSAPAVPVTTTSPATDSKLQATEVKS
VPVAQKAPTATPVAQPASTTNAVAAHPENAGLQPHVAAYKEKVASTYGVN
EFSTYRAGDPGDHGKGLAVDFIVGTNQALGNKVAQYSTQNMAANNISYVI
WQQKFYSNTNSIYGPANTWNAMPDRGGVTANHYDHVHVSFNK

Additional preferred fragments of GBS 322 comprise the immunogenic epitopes identified in WO 03/068813, each of which are specifically incorporated by reference herein.

There may be an upper limit to the number of GBS proteins which will be in the compositions of the invention. Preferably, the number of GBS proteins in a composition of the invention is less than 20, less than 19, less than 18, less than 17, less than 16, less than 15, less than 14, less than 13, less than 12, less than less than 10, less than 9, less than 8, less than 7, less than 6, less than 5, less than 4, or less than 3. Still more preferably, the number of GBS proteins in a composition of the invention is less than 6, less than 5, or less than 4. Still more preferably, the number of GBS proteins in a composition of the invention is 3.

The GBS proteins and polynucleotides used in the invention are preferably isolated, i.e., separate and discrete, from the whole organism with which the molecule is found in nature or, when the polynucleotide or polypeptide is not found in nature, is sufficiently free of other biological macromolecules so that the polynucleotide or polypeptide can be used for its intended purpose.

### Group A Streptococcus Adhesin Island Sequences

The GAS AI polypeptides of the invention can, of course, be prepared by various means (e.g. recombinant expression, purification from GAS, chemical synthesis etc.) and in various forms (e.g. native, fusions, glycosylated, non-glycosylated etc.). They are preferably prepared in substantially pure form (i.e. substantially free from other streptococcal or host cell proteins) or substantially isolated form.

The GAS AI proteins of the invention may include polypeptide sequences having sequence identity to the identified GAS proteins. The degree of sequence identity may vary depending on the amino acid sequence (a) in question, but is preferably greater than 50% (e.g. 60%, 65%, 70%, 75%, 80%, 85%, 90%, 91%, 92%, 93%, 94%, 95%, 96%, 97%, 98%, 99%, 99.5% or more). Polypeptides having sequence identity include homologs, orthologs, allelic variants and functional mutants of the identified GBS proteins. Typically, 50% identity or more between two proteins is considered to be an indication of functional equivalence. Identity between proteins is preferably determined by the Smith-Waterman homology search algorithm as implemented in the MPSRCH program (Oxford Molecular), using an affinity gap search with parameters gap open penalty=12 and gap extension penalty=1.

The GAS adhesin island polynucleotide sequences may include polynucleotide sequences having sequence identity to the identified GAS adhesin island polynucleotide sequences. The degree of sequence identity may vary depending on the polynucleotide sequence in question, but is preferably greater than 50% (e.g. 60%, 65%, 70%, 75%, 80%, 85%, 90%, 91%, 92%, 93%, 94%, 95%, 96%, 97%, 98%, 99%, 99.5% or more).

The GAS adhesin island polynucleotide sequences of the invention may include polynucleotide fragments of the identified adhesin island sequences. The length of the fragment may vary depending on the polynucleotide sequence of the specific adhesin island sequence, but the fragment is preferably at least 10 consecutive polynucleotides, (e.g. at least 10, 12, 14, 16, 18, 20, 25, 30, 35, 40, 50, 60, 70, 80, 90, 100, 150, 200 or more).

The GAS adhesin island amino acid sequences of the invention may include polypeptide fragments of the identified GAS proteins. The length of the fragment may vary depending on the amino acid sequence of the specific GAS antigen, but the fragment is preferably at least 7 consecutive amino acids, (e.g. 8, 10, 12, 14, 16, 18, 20, 25, 30, 35, 40, 50, 60, 70, 80, 90, 100, 150, 200 or more). Preferably the fragment comprises one or more epitopes from the sequence. Other preferred fragments include (1) the N-terminal signal peptides of each identified GAS protein, (2) the identified GAS protein without their N-terminal signal peptides, and (3) each identified GAS protein wherein up to 10 amino acid residues (e.g. 1, 2, 3, 4, 5, 6, 7, 8, 9, 10, 15, 20, 25 or more) are deleted from the N-terminus and/or the C-terminus e.g. the N-terminal amino acid residue may be deleted. Other fragments omit one or more domains of the protein (e.g. omission of a signal peptide, of a cytoplasmic domain, of a transmembrane domain, or of an extracellular domain).

GAS AI-1 sequences

As discussed above, a GAS AI-1 sequence is present in an M6 strain isolate (MGAS10394). Examples of GAS AI-1 sequences from M6 strain isolate MGAS 10394 are set forth below.

M6_Spy0156: Spy0156 is a rofA transcriptional regulator. An example of an amino acid sequence for M6_Spy0156 is set forth in SEQ ID NO:41.

SEQ ID NO: 41

MIEKYLESSIESKCQLVVLFFKTSYLPITEVAEKTGLTFLQLNHYCEELN
AFFPDSLSMTIQKRMISCQFTHPFKETYLYQLYASSNVLQLLAFLIKNGS
HSRPLTDFARSHFLSNSSAYRMREALIPLLRNFELKLSKNKIVGEEYRIR
YLIALLYSKFGIKVYDLTQQDKNTIHSFLSHSSTHLKTSPWLSESFSFYD
ILLALSWKRHQFSVTIPQTRIFQQLKKLFIYDSLKKSSRDIIETYCQLNF
SAGDLDYLYLIYITANNSFASLQWTPEHIRQCCQLFEENDTFRLLLKPII
TLLPNLKEQKPSLVKALMFFSKSFLFNLQHFIPETNLFVSPYYKGNQKLY
TSLKLIVEEWLAKLPGKRYLNHKHFHLFCHYVEQILRNIQPPLVVVFVAS
NFINAHLLTDSFPRYFSDKSIDFHSYIAR

M6_Spy0157: M6_Spy0157 is a fibronectin binding protein. It contains a sortase substrate motif LPXTG (SEQ ID NO:122), shown in italics in the amino acid sequence SEQ ID NO:42.

SEQ ID NO: 42

MVSSYMFVRGEKMNNKIFLNKEASFLAHTKRKRRFAVTLVGVFFMLLACA
GAIGFGQVAYAADEKTVPSHSSPNPEFPWYGYDAYGKEYPGYNIWTRYHD
LRVNLNGSRSYQVYCFNIQSNYPSQKNSFIKNWFKKIEGNGKSFVDYAHT
TKLGKEELEQRLLSLLYNAYPNDANGYMKGLEHLNAITVTQYAVWHYSDN
SQYQFETLWESEAKEGKISRSQVTLMREALKKLIDPNLEATAVNKIPSGY
RLNIFESENEAYQNLLSAEYVPDDPPKPGETSEHNPKTPELDGTPIPEDP
KHPDDNLEPTLPPVMLDGEEVPEVPSESLEPALPPLMPELDGQEVPEKPS
IDLPIEVPRYEFNNKDQSPLAGESGETEYITEVYGNQQNPVDIDKKLPNE
TGFSGNMVETEDTKEPEVLMGGQSESVEFTKDTQTGMSGQTTPQVETEDT
KEPEVLMGGQSESVEFTKDTQTGMSGQTTPQIETEDTKEPEVLMGGQSES
VEFTKDTQTGMSGQTTPQIETEDTKEPEVLMGGQSESVEFTKDTQTGMSG
FSETATVVEDTRPKLVFHFDNNEPKVEENREKPTKNITPILPATGDIENV
LAFLGILILSVLSIFSLLKNKQSNKKV

M6_Spy0157 contains an amino acid motif indicative of a cell wall anchor: SEQ ID NO:180 LPATG (shown in italics in SEQ ID NO:42, above). In some recombinant host cell systems, it may be preferable to remove this motif to facilitate secretion of a recombinant M6_Spy0157 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in M6_Spy0157. The pilin motif sequence is underlined in SEQ ID NO:42, below. Conserved lysine (K) residues are also marked in bold, at amino acid residues 277, 287, and 301. The pilin sequence, in particular the conserved lysine residues, are thought to be important for the formation of oligomeric, pilus-like structures. Preferred fragments of M6_Spy0157 include at least one conserved lysine residue. Preferably, fragments include the pilin sequence.

SEQ ID NO: 42

MVSSYMFVRGEKMNNKIFLNKEASFLAHTKRKRRFAVTLVGVFFMLLACA
GAIGFGQVAYAADEKTVPSHSSPNPEFPWYGYDAYGKEYPGYNIWTRYHD
LRVNLNGSRSYQVYCFNIQSNYPSQKNSFIKNWFKKIEGNGKSFVDYAHT
TKLGKEELEQRLLSLLYNAYPNDANGYMKGLEHLNAITVTQYAVWHYSDN
SQYQFETLWESEAKEGKISRSQVTLMREALKKLIDPNLEATAVNKIPSGY
RLNIFESENEAYQNLLSAEYVPDDPPKPGETSEHNPKTPELDGTPIPEDP
KHPDDNLEPTLPPVMLDGEEVPEVPSESLEPALPPLMPELDGQEVPEKPS
IDLPIEVPRYEFNNKDQSPLAGESGETEYITEVYGNQQNPVDIDKKLPNE
TGFSGNMVETEDTKEPEVLMGGQSESVEFTKDTQTGMSGQTTPQVETEDT
KEPEVLMGGQSESVEFTKDTQTGMSGQTTPQIETEDTKEPEVLMGGQSES
VEFTKDTQTGMSGQTTPQIETEDTKEPEVLMGGQSESVEFTKDTQTGMSG
FSETATVVEDTRPKLVFHFDNNEPKVEENREKPTKNITPILPATGDIENV
LAFLGILILSVLSIFSLLKNKQSNKKV

A repeated series of four E boxes containing a conserved glutamic residue have been identified in M6_Spy0157. The E-box motifs are underlined in SEQ ID NO:42, below. The conserved glutamic acid (E) residues, at amino acid residues 415, 452, 489, and 526 are marked in bold. The E box motif, in particular the conserved glutamic acid residue, is thought to be important for the formation of oligomeric pilus-like structures of M6_Spy0157. Preferred fragments of M6_Spy0157 include at least one conserved glutamic acid residue. Preferably, fragments include at least one E box motif.

SEQ ID NO: 42

MVSSYMFVRGEKMNNKIFLNKEASFLAHTKRKRRFAVTLVGVFFMLLACA
GAIGFGQVAYAADEKTVPSHSSPNPEFPWYGYDAYGKEYPGYNIWTRYHD
LRVNLNGSRSYQVYCFNIQSNYPSQKNSFIKNWFKKIEGNGKSFVDYAHT
TKLGKEELEQRLLSLLYNAYPNDANGYMKGLEHLNAITVTQYAVWHYSDN
SQYQFETLWESEAKEGKISRSQVTLMREALKKLIDPNLEATAVNKIPSGY
RLNIFESENEAYQNLLSAEYVPDDPPKPGETSEHNPKTPELDGTPIPEDP
KHPDDNLEPTLPPVMLDGEEVPEVPSESLEPALPPLMPELDGQEVPEKPS
IDLPIEVPRYEFNNKDQSPLAGESGETEYITEVYGNQQNPVDIDKKLPNE
TGFSGNMVETEDTKEPEVLMGGQSESVEFTKDTQTGMSGQTTPQVETEDT
KEPEVLMGGQSESVEFTKDTQTGMSGQTTPQIETEDTKEPEVLMGGQSES
VEFTKDTQTGMSGQTTPQIETEDTKEPEVLMGGQSESVEFTKDTQTGMSG
FSETATVVEDTRPKLVFHFDNNEPKVEENREKPTKNITPILPATGDIENV
LAFLGILILSVLSIFSLLKNKQSNKKV

M6_Spy0158: M6_Spy0158 is a reverse transcriptase. An example of Spy0158 is shown in the amino acid sequence SEQ ID NO 43.

SEQ ID NO: 43

MSLRHQNKKGIRKEGWKSRPQSRWSDHCQLVAQKSVLKQAISKTVLA
ERGLFSCLDDYLERHALKVN

M6_Spy0159: M6_Spy0159 is a collagen adhesion protein. It contains a sortase substrate motif LPXSG, shown in italics in the amino acid sequence SEQ ID NO:44.

SEQ ID NO: 44

MYSRLKRELVIVINRKKKYKLIRLMVTVGLIFSQLVLPIRRLGLQMISTQ
TKVIPQEIVTQTETQGTQVVATKQKLESENSSLKVALKRESGFEHNATID
ASLDTESQGDNSQRSVTQAIVTMALELRKQGLSIVDTKIVRIQSSTNQRN
DITTTLTFKNGLSLEGASTEANDPNVRVGIVNPNDTVQTITPTIKQDADG
KVKNLVFTGRLGKQVIIVSTTRLKEEQTISLDSYGELVIDGAVGLSQKDR
PPYSKPITVNILKPKLSSIESSLDSKDFEIVKTIDNLYTWDDQFYLLDFI
SKQYEVLKTDYQSAKDSTPQTRDILFGEYTVEPLVMNKGHNNTINIYIRS
TRPLGLKPIGAAPALIQPRSFRSLTPRSTRMKRSAPVEKFEGELEHHKRI
DYLGDNQNNPDTTIDDKEDEHDTSDLYRLYLDMTGKKNPLDILVVVDKSG
SMQEGIGSVQRYRYYAQRWDDYYSQWVYHGTFDYSSYQGESFNRGQIHYR
YRGIVSVSDGIRRDDAVKNSLLGVNGLLQRFVNINPENKLSVIGFQGSAD
YHAGKWYPDQSPRGGFYQPNLNNSRDAELLKGWSTNSLLDPNTLTALHNN
GTNYHAALLKAKEILNEVKDDGRRKIMIFISDGVPTFYFGEDGYRSGNGS
SNDRNNVTRSQEGSKLAIDEFKARYPNLSIYSLGVSKDINSDTASSSPVV
LKYLSGEEHYYGITDTAELEKTLNKIVEDSKLSQLGISDSLSQYVDYYDK
QPDVLVTRKSKVNDETEILYQKDQVQEAGKDIIDKVVFTPKTTSQPKGKV
TLTFKSDYKVDDEYTYTLSFNVKASDEAYEKYKDNEGRYSEMGDSDTDYG
TNQTSSGKGGLPSNSDASVNYMADGREQKLPYKHPVIQVKTVPITFTKVD
ADNNQKKLAGVEFELRKEDKKIVWEKGTTGSNGQLNFKYLQKGKTYYLYE
TKAKLGYTLPENPWEVAVANNGDIKVKHPIEGELKSKDGSYMIKNYKTYQ
LPSSGGRGSQIFIIVGSMTATVALLFYRRQHRKKQY

M6 Spy0159 contains an amino acid motif indicative of a cell wall anchor: SEQ ID NO:181 LPSSG (shown in italics in SEQ ID NO:44, above). In some recombinant host cell systems, it may be preferable to remove this motif to facilitate secretion of a recombinant M6_Spy0159 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in M6_Spy0159. The pilin motif sequence is underlined in SEQ ID NO:44, below. Conserved lysine (K) residues are also marked in bold, at amino acid residues 265 and 276. The pilin sequence, in particular the conserved lysine residues, are thought to be important for the formation of oligomeric, pilus-like structures. Preferred fragments of M6_Spy0159 include at least one conserved lysine residue. Preferably, fragments include the pilin sequence.

SEQ ID NO: 44

MYSRLKRELVIVINRKKKYKLIRLMVTVGLIFSQLVLPIRRLGLQMISTQ
TKVIPQEIVTQTETQGTQVVATKQKLESENSSLKVALKRESGFEHNATID
ASLDTESQGDNSQRSVTQAIVTMALELRKQGLSIVDTKIVRIQSSTNQRN
DITTTLTFKNGLSLEGASTEANDPNVRVGIVNPNDTVQTITPTIKQDADG
KVKNLVFTGRLGKQVIIVSTTRLKEEQTISLDSYGELVIDGAVGLSQKDR
PPYSKPITVNILKPKLSSIESSLDSKDFEIVKTIDNLYTWDDQFYLLDFI
SKQYEVLKTDYQSAKDSTPQTRDILFGEYTVEPLVMNKGHNNTINIYIRS
TRPLGLKPIGAAPALIQPRSFRSLTPRSTRMKRSAPVEKFEGELEHHKRI
DYLGDNQNNPDTTIDDKEDEHDTSDLYRLYLDMTGKKNPLDILVVVDKSG
SMQEGIGSVQRYRYYAQRWDDYYSQWVYHGTFDYSSYQGESFNRGQIHYR
YRGIVSVSDGIRRDDAVKNSLLGVNGLLQRFVNINPENKLSVIGFQGSAD
YHAGKWYPDQSPRGGFYQPNLNNSRDAELLKGWSTNSLLDPNTLTALHNN
GTNYHAALLKAKEILNEVKDDGRRKIMIFISDGVPTFYFGEDGYRSGNGS
SNDRNNVTRSQEGSKLAIDEFKARYPNLSIYSLGVSKDINSDTASSSPVV
LKYLSGEEHYYGITDTAELEKTLNKIVEDSKLSQLGISDSLSQYVDYYDK
QPDVLVTRKSKVNDETEILYQKDQVQEAGKDIIDKVVFTPKTTSQPKGKV
TLTFKSDYKVDDEYTYTLSFNVKASDEAYEKYKDNEGRYSEMGDSDTDYG
TNQTSSGKGGLPSNSDASVNYMADGREQKLPYKHPVIQVKTVPITFTKVD
ADNNQKKLAGVEFELRKEDKKIVWEKGTTGSNGQLNFKYLQKGKTYYLYE
TKAKLGYTLPENPWEVAVANNGDIKVKHPIEGELKSKDGSYMIKNYKIYQ
LPSSGGRGSQIFIIVGSMTATVALLFYRRQHRKKQY

An E box containing a conserved glutamic residue has been identified in M6_Spy0159. The E-box motif is underlined in SEQ ID NO:44, below. The conserved glutamic acid (E), at amino acid residue 950, is marked in bold. The E box motif, in particular the conserved glutamic acid residue, is thought to be important for the formation of oligomeric pilus-like structures of M6_Spy0159. Preferred fragments of M6_Spy0159 include the conserved glutamic acid residue. Preferably, fragments include the E box motif.

SEQ ID NO: 44

MYSRLKRELVIVINRKKKYKLIRLMVTVGLIFSQLVLPIRRLGLQMISTQ
TKVIPQEIVTQTETQGTQVVATKQKLESENSSLKVALKRESGFEHNATID
ASLDTESQGDNSQRSVTQAIVTMALELRKQGLSIVDTKIVRIQSSTNQRN
DITTTLTFKNGLSLEGASTEANDPNVRVGIVNPNDTVQTITPTIKQDADG
KVKNLVFTGRLGKQVIIVSTTRLKEEQTISLDSYGELVIDGAVGLSQKDR
PPYSKPITVNILKPKLSSIESSLDSKDFEIVKTIDNLYTWDDQFYLLDFI
SKQYEVLKTDYQSAKDSTPQTRDILFGEYTVEPLVMNKGHNNTINIYIRS
TRPLGLKPIGAAPALIQPRSFRSLTPRSTRMKRSAPVEKFEGELEHHKRI
DYLGDNQNNPDTTIDDKEDEHDTSDLYRLYLDMTGKKNPLDILVVVDKSG
SMQEGIGSVQRYRYYAQRWDDYYSQWVYHGTFDYSSYQGESFNRGQIHYR
YRGIVSVSDGIRRDDAVKNSLLGVNGLLQRFVNINPENKLSVIGFQGSAD
YHAGKWYPDQSPRGGFYQPNLNNSRDAELLKGWSTNSLLDPNTLTALHNN
GTNYHAALLKAKEILNEVKDDGRRKIMIFISDGVPTFYFGEDGYRSGNGS
SNDRNNVTRSQEGSKLAIDEFKARYPNLSIYSLGVSKDINSDTASSSPVV
LKYLSGEEHYYGITDTAELEKTLNKIVEDSKLSQLGISDSLSQYVDYYDK
QPDVLVTRKSKVNDETEILYQKDQVQEAGKDIIDKVVFTPKTTSQPKGKV
TLTFKSDYKVDDEYTYTLSFNVKASDEAYEKYKDNEGRYSEMGDSDTDYG
TNQTSSGKGGLPSNSDASVNYMADGREQKLPYKHPVIQVKTVPITFTKVD
ADNNQKKLAGVEFELRKEDKKIVWEKGTTGSNGQLNFKYLQKGKTYYLYE
TKAKLGYTLPENPWEVAVANNGDIKVKHPIEGELKSKDGSYMIKNYKIYQ
LPSSGGRGSQIFIIVGSMTATVALLFYRRQHRKKQY

M6_Spy0160: M6_Spy0160 is a fimbrial structural subunit. It contains a sortase substrate motif LPXTG (SEQ ID NO:122), shown in italics in amino acid sequence SEQ ID NO:45.

SEQ ID NO: 45

MTNRRETVREKILITAKKLMLACLAILAVVGLGMTRVSALSKDDTAQLKI
TNIEGGPTVTLYKIGEGVYNTNGDSFINFKYAEGVSLTETGPTSQEITTI
ANGINTGKIKPFSTENVSISNGTATYNARGASVYIALLTGATDGRTYNPI
LLAASYNGEGNLVTKNIDSKSNYLYGQTSVAKSSLPSITKKVTGTIDDVN
KKTTSLGSVLSYSLTFELPSYTKEAVNKTVYVSDNMSEGLTFNFNSLTVE
WKGKMANITEDGSVMVENTKIGIAKEVNNGFNLSFIYDSLESISPNISYK
AVVNNKAIVGEEGNPNKAEFFYSNNPTKGNTYDNLDKKPDKGNGITSKED
SKIVYTYQIAFRKVDSVSKTPLIGAIFGVYDTSNKLIDIVTTNKNGYAIS
TQVSSGKYKIKELKAPKGYSLNTETYEITANWVTATVKTSANSKSTTYTS
DKNKATDNSEQVGWLKNGIFYSIDSRPTGNDVKEAYIESTKALTDGTTFS
KSNEGSGTVLLETDIPNTKLGELPSTGSIGTYLFKAIGSAAMIGAIGIYI
VKRRKA

M6_Spy0160 contains an amino acid motif indicative of a cell wall anchor: SEQ ID NO:131 LPSTG (shown in italics in SEQ ID NO:45, above). In some recombinant host cell systems, it may be preferable to remove this motif to facilitate secretion of a recombinant M6_Spy0160 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

An E box containing a conserved glutamic residue has been identified in M6_Spy0160. The E-box motif is underlined in SEQ ID NO:45, below. The conserved glutamic acid (E), at amino acid residue 412, is marked in bold. The E box motif, in particular the conserved glutamic acid residue, is thought to be important for the formation of oligomeric pilus-like structures of M6_Spy0160. Preferred fragments of M6_Spy0160 include the conserved glutamic acid residue. Preferably, fragments include the E box motif.

SEQ ID NO: 45

MTNRRETVREKILITAKKLMLACLAILAVVGLGMTRVSALSKDDTAQLKI
TNIEGGPTVTLYKIGEGVYNTNGDSFINFKYAEGVSLTETGPTSQEITTI
ANGINTGKIKPFSTENVSISNGTATYNARGASVYIALLTGATDGRTYNPI
LLAASYNGEGNLVTKNIDSKSNYLYGQTSVAKSSLPSITKKVTGTIDDVN
KKTTSLGSVLSYSLTFELPSYTKEAVNKTVYVSDNMSEGLTFNFNSLTVE
WKGKMANITEDGSVMVENTKIGIAKEVNNGFNLSFIYDSLESISPNISYK
AVVNNKAIVGEEGNPNKAEFFYSNNPTKGNTYDNLDKKPDKGNGITSKED
SKIVYTYQIAFRKVDSVSKTPLIGAIFGVYDTSNKLIDIVTTNKNGYAIS
TQVSSGKYKIKELKAPKGYSLNTETYEITANWVTATVKTSANSKSTTYTS
DKNKATDNSEQVGWLKNGIFYSIDSRPTGNDVKEAYIESTKALTDGTTFS
KSNEGSGTVLLETDIPNTKLGELPSTGSIGTYLFKAIGSAAMIGAIGIYI
VKRRKA

M6_Spy0161 is a srtB type sortase. An example of an amino acid sequence of M6_Spy-161 is shown in SEQ ID NO:46.

SEQ ID NO: 46

MTERLKNLGILLLFLLGTAIFLYPTLSSQWNAYRDRQLLSTYHKQVIQKK
PSEMEEVWQKAKAYNARLGIQPVPDAFSFRDGIHDKNYESLLQIENNDIM
GYVEVPSIKVTLPIYHYTTDEVLTKGAGHLFGSALPVGGDGTHTVISAHR
GLPSAEMFTNLNLVKKGDTFYFRVLNKVLAYKVDQILIVEPDQATSLSGV
MGKDYATLVTCTPYGVNTKRLLVRGHRIAYHYKKYQQAKKAMKLVDKSRM
WAEVVCAAFGVVIAIILVFMYSRVSAKKSK

As discussed above, applicants have also determined the nucleotide and encoded amino acid sequence of fimbrial structural subunits in several other GAS AI-1 strains of bacteria. Examples of sequences of these fimbrial structural subunits are set forth below.

M6 strain isolate CDC SS 410 is a GAS AI-1 strain of bacteria. CDC SS 410_fimbrial is thought to be a fimbrial structural subunit of M6 strain isolate CDC SS 410. An example of a nucleotide sequence encoding the CDC SS 410_fimbrial protein (SEQ ID NO:267) and a CDC SS 410_fimbrial protein amino acid sequence (SEQ ID NO:268) are set forth below.

SEQ ID NO: 267

AAAGATGATACTGCACAACTAAAGATAACAAATATTGAAGGTGGGCCAAC
AGTAACACTTTATAAAATAGGAGAAGGTGTTTACAACACTAATGGTGATT
CTTTTATTAACTTTAAATATGCTGAGGGGGTTTCTTTAACTGAAACAGGA
CCTACATCACAAGAAATTACTACTATTGCAAATGGTATTAATACGGGTAA
AATAAAGCCTTTTAGTACTGAAAACGTTAGTATTTCTAATGGAACAGCAA
CTTATAATGCGAGAGGTGCATCTGTTTATATTGCATTATTAACAGGTGCG
ACAGATGGCCGTACCTACAATCCTATTTTATTAGCTGCATCTTATAATGG
TGAGGGAAATTTAGTTACTAAAAATATTGATTCCAAATCTAATTATTTAT
ATGGACAAACAAGTGTTGCAAAATCATCATTACCATCTATTACAAAGAAA
GTAACCGGGACAATAGATGACGTGAATAAAAAGACTACCTCGTTAGGAAG
TGTATTGTCTTATTCGCTGACATTTGAATTACCAAGTTATACCAAAGAAG
CAGTCAATAAAACAGTATATGTTTCTGATAATATGTCGGAAGGTCTTACT
TTTAACTTTAATAGTCTTACAGTAGAATGGAAAGGTAAGATGGCTAATAT
TACTGAAGATGGTTCAGTAATGGTAGAAAATACAAAAATCGGAATAGCTA
AGGAGGTTAATAACGGTTTTAATTTAAGTTTTATTTATGATAGTTTAGAA
TCTATATCACCAAATATAAGTTATAAAGCTGTTGTAAACAATAAAGCTAT
TGTTGGTGAAGAGGGTAATCCTAATAAAGCTGAATTCTTCTATTCAAATA
ATCCAACAAAAGGTAATACATACGATAATTTAGATAAGAAGCCTGATAAA
GGGAATGGTATTACATCCAAAGAAGATTCTAAAATTGTTTATACTTATCA
AATAGCGTTTAGAAAAGTTGATAGTGTTAGTAAGACCCCACTTATTGGTG
CAATTTTTGGAGTTTATGATACTAGTAATAAATTAATTGATATTGTTACA
ACCAATAAAAATGGATATGCTATTTCAACACAAGTATCTTCAGGAAAATA
TAAAATTAAGGAATTAAAAGCTCCTAAAGGTTATTCATTGAATACAGAAA
CTTATGAAATTACGGCAAATTGGGTAACTGCTACAGTCAAGACAAGTGCT
AATTCAAAAAGTACTACTTATACATCTGATAAAAATAAGGCGACAGATAA
TTCAGAGCAAGTAGGATGGTTAAAAAATGGTATATTCTATTCTATAGATA
GTAGACCTACAGGAAATGATGTTAAAGAGGCTTATATTGAATCTACTAAG
GCTTTAACTGATGGAACAACTTTCTCAAAATCGAATGAAGGTTCAGGTAC
AGTATTATTAGAAACTGACATCCCTAACACCAAGCTAGGTGAACTC

SEQ ID NO: 268

KDDTAQLKITNIEGGPTVTLYKIGEGVYNTNGDSFINFKYAEGVSLTETG
PTSQEITTIANGINTGKIKPFSTENVSISNGTATYNARGASVYIALLTGA
TDGRTYNPILLAASYNGEGNLVTKNIDSKSNYLYGQTSVAKSSLPSITKK
VTGTIDDVNKKTTSLGSVLSYSLTFELPSYTKEAVNKTVYVSDNMSEGLT
FNFNSLTVEWKGKMANITEDGSVMVENTKIGIAKEVNNGFNLSFIYDSLE
SISPNISYKAVVNNKAIVGEEGNPNKAEFFYSNNPTKGNTYDNLDKKPDK
GNGITSKEDSKIVYTYQIAFRKVDSVSKTPLIGAIFGVYDTSNKLIDIVT
TNKNGYAISTQVSSGKYKIKELKAPKGYSLNTETYEITANWVTATVKTSA
NSKSTTYTSDKNKATDNSEQVGWLKNGIFYSIDSRPTGNDVKEAYIESTK
ALTDGTTFSKSNEGSGTVLLETDIPNTKLGEL

M6 strain isolate ISS 3650 is a GAS AI-1 strain of bacteria. ISS3650_fimbrial is thought to be a fimbrial structural subunit of M6 strain isolate ISS 3650. An example of a nucleotide sequence encoding the ISS3650_fimbrial protein (SEQ ID NO:269) and an ISS3650_fimbrial protein amino acid sequence (SEQ ID NO:270) are set forth below.

SEQ ID NO: 269

GAATGGAAAGGTAAGATGGCTAATATTACTGAAGATGGTTCAGTAATGGT
AGAAAATACAAAAATCGGAATAGCTAAGGAGGTTAATAACGGTTTTAATT
TAAGTTTTATTTATGATAGTTTAGAATCTATATCACCAAATATAAGTTAT
AAAGCTGTTGTAAACAATAAAGCTATTGTTGGTGAAGAGGGTAATCCTAA
TAAAGCTGAATTCTTCTATTCAAATAATCCAACAAAAGGTAATACATACG
ATAATTTAGATAAGAAGCCTGATAAAGGGAATGGTATTACATCCAAAGAA
GATTCTAAAATTGTTTATACTTATCAAATAGCGTTTAGAAAAGTTGATAG
TGTTAGTAAGACCCCACTTATTGGTGCAATTTTTGGAGTTTATGATACTA
GTAATAAATTAATTGATATTGTTACAACCAATAAAAATGGATATGCTATT
TCAACACAAGTATCTTCAGGAAAATATAAAATTAAGGAATTAAAAGCTCC
TAAAGGTTATTCATTGAATACAGAAACTTATGAAATTACGGCAAATTGGG
TAACTGCTACAGTCAAGACAAGTGCTAATTCAAAAAGTACTACTTATACA
TCTGATAAAAATAAGGCGACAGATAATTCAGAGCAAGTAGGATGGTTAAA
AAATGGTATATTCTATTCTATAGATAGTAGACCTACAGGAAATGATGTTA
AAGAGGCTTATATTGAATCTACTAAGGCTTTAACTGATGGAACAACTTTC
TCAAAATCGAATGAAGGTTCAGGTACAGTATTATTAGAAACTGACATCC

SEQ ID NO: 270

EWKGKMANITEDGSVMVENTKIGIAKEVNNGFNLSFIYDSLESISPNISY
KAVVNNKAIVGEEGNPNKAEFFYSNNPTKGNTYDNLDKKPDKGNGITSKE
DSKIVYTYQIAFRKVDSVSKTPLIGAIFGVYDTSNKLIDIVTTNKNGYAI
STQVSSGKYKIKELKAPKGYSLNTETYEITANWVTATVKTSANSKSTTYT
SDKNKATDNSEQVGWLKNGIFYSIDSRPTGNDVKEAYIESTKALTDGTTF
SKSNEGSGTVLLETDI

M23 strain isolate DSM2071 is a GAS AI-1 strain of bacteria. DSM2071 fimbrial is thought to be a fimbrial structural subunit of M23 strain DSM2071. An example of a nucleotide sequence encoding the DSM2071_fimbrial protein (SEQ ID NO:251) and a DSM2071_fimbrial protein amino acid sequence (SEQ ID NO:252) are set forth below.

SEQ ID NO: 251

ATGAGAGAGAAAATATTAATAGCAGCAAAAAAACTAATGCTAGCTTGTTT
AGCTATCTTAGCTGTAGTAGGGCTTGGAATGACAAGAGTATCAGCTTTAT
CAAAAGATGATAAGGCGGAGTTGAAGATAACAAATATCGAAGGTAAACCG
ACCGTGACACTGTATAAAATTGGTGATGGAAAATACAGTGAGCGAGGGGA
TTCTTTTATTGGATTTGAGTTAAAGCAAGGTGTGGAGCTAAATAAGGCAA
AACCTACATCTCAAGAAATAAATAAAATCGCTAATGGTATTAATAAAGGT
AGTGTTAAGGCTGAAGTAGTTAATATAAAAGAACATGCTAGTACAACTTA
TAGTTATACAACAACTGGTGCAGGTATTTACTTGGCTATATTGACTGGAG
CTACTGATGGACGTGCCTATAATCCTATCTTACTGACAGCTTCTTACAAT
GAGGAAAATCCACTTAAGGGAGGGCAGATTGACGCAACTAGTCATTATCT
TTTTGGAGAAGAAGCAGTTGCTAAATCTAGCCAACCAACAATTAGCAAGT
CAATTACAAAATCCACAAAAGATGGTGATAAAGATACAGCATCTGTAGGT
GAAAAAGTTGATTACAAATTAACTGTTCAGTTACCAAGTTATTCGAAAGA
TGCTATCAATAAAACGGTGTTTATCACTGACAAATTGTCTCAGGGACTTA
CTTTCCTTCCAAAAAGTTTAAAGATTATCTGGAATGGTCAAACGTTAACA
AAGGTGAATGAAGAATTTAAAGCTGGAGATAAGGTAATTGCTCAACTTAA
GGTTGAAAATAATGGATTTAATCTGAACTTTAATTATGATAACCTTGATA
ATCATGCCCCAGAAGTTAACTATAGTGCTCTACTAAATGAAAACGCAGTT
GTTGGTAAAGGTGGTAATGACAATAATGTAGACTATTACTATTCAAATAA
TCCGAATAAAGGAGAGACCCATAAAACAACTGAGAAGCCTAAAGAGGGTG
AAGGTACTGGTATCACTAAAAAGACGGATAAAAAAACCGTCTACACCTAT
CGTGTAGCCTTTAAGAAAACAGGCAAAGATCATGCCCCACTAGCTGGTGC
TGTTTTCGGTATCTATTCAGATAAGGAAGCGAAACAATTAGTCGATATTG
TTGTGACAAATGCACAGGGTTATGCAGCATCAAGCGAAGTTGGGAAAGGG
ACTTATTACATTAAAGAAATTAAATCCCCTAAGGGTTACTCTTTAAATAC
AAATATTTATGAAGTGGAAACTTCATGGGAAAAAGCTACAACGACTTCTA
CAACTAATCGTTTAGAGACAATTTATACAACAGATGATAATCAAAAGTCT
CCAGGAACTAATACAGTTGGTTGGTTGGAAGATGGTGTCTTTTACAAAGA
AAATCCAGGTGGTGATGCTAAACTTGCCTATATCAAACAATCAACAGAGG
AGACTTCTACAACTATAGAAGTCAAAGAAAATCAAGCTGAAGGTTCAGGT
ACGGTATTATTAGAAACTGAAATTCCTAACACCAAATTAGGTGAATTACC
TTCGACAGGTAGCATTGGTACTTACCTCTTTAAAGCTATTGGTTCGGCTG
CTATGATCGGTGCAATTGGTATTTATATTGTTAAACGTCGTAAAGCTTAA

SEQ ID NO: 252

MREKILIAAKKLMLACLAILAVVGLGMTRVSALSKDDKAELKITNIEGKP
TVTLYKIGDGKYSERGDSFIGFELKQGVELNKAKPTSQEINKIANGINKG
SVKAEVVNIKEHASTTYSYTTTGAGIYLAILTGATDGPAYNPILLTASYN
EENPLKGGQIDATSHYLFGEEAVAKSSQPTISKSITKSTKDGDKDTASVG
EKVDYKLTVQLPSYSKDAINKTVFITDKLSQGLTFLPKSLKIIWNGQTLT
KVNEEFKAGDKVIAQLKVENNGFNLNFNYDNLDNHAPEVNYSALLNENAV
VGKGGNDNNVDYYYSNNPNKGETHKTTEKPKEGEGTGITKKTDKKTVYTY
RVAFKKTGKDHAPLAGAVFGIYSDKEAKQLVDIVVTNAQGYAASSEVGKG
TYYIKEIKSPKGYSLNTNIYEVETSWEKATTTSTTNRLETIYTTDDNQKS
PGTNTVGWLEDGVFYKENPGGDAKLAYIKQSTEETSTTIEVKENQAEGSG
TVLLETEIPNTKLGELPSTGSIGTYLFKAIGSAAMIGAIGIYIVKRRKA

GAS AI-2 Sequences

As discussed above, a GAS AI-2 sequence is present in an M1 strain isolate (SF370). Examples of GAS AI-2 sequences from M1 strain isolate SF370 are set forth below.

Spy0124 is a rofA transcriptional regulator. An example of an amino acid sequence for Spy0124 is set forth in SEQ ID NO:47.

SEQ ID NO: 47

MIEKYLESSIESKCQLIVLFFKTSYLPITEVAEKTGLTFLQLNHYCEELN
AFFPGSLSMTIQKRMISCQFTHPFKETYLYQLYASSNVLQLLAFLIKNGS
HSRPLTDFARSHFLSNSSAYRMREALIPLLRNFELKLSKNKIVGEEYRIR
YLIALLYSKFGIKVYDLTQQDKNTIHSFLSHSSTHLKTSPWLSESFSFYD
ILLALSWKRHQFSVTIPQTRIFQQLKKLFVYDSLKKSSHDIIETYCQLNF
SAGDLDYLYLIYITANNSFASLQWTPEHIRQYCQLFEENDTFRLLLNPII
TLLPNLKEQKASLVKALMFFSKSFLFNLQHFIPETNLFVSPYYKGNQKLY
TSLKLIVEEWMAKLPGKRDLNHKHFHLFCHYVEQSLRNIQPPLVVVFVAS
NFINAHLLTDSFPRYFSDKSIDFHSYYLLQDNVYQIPDLKPDLVITHSQL
IPFVHHELTKGIAVAEISFDESILSIQELMYQVKEEKFQADLTKQLT

GAS 015 is also referred to as Cpa. It contains a sortase substrate motif VVXTG (SEQ ID NO:135), shown in italics in SEQ ID NO:48.

SEQ ID NO: 48

LRGEKMKKTRFPNKLNTLNTQRVLSKNSKRFTVTLVGVFLMIFALVTSMV
GAKTVFGLVESSTPNAINPDSSSEYRWYGYESYVRGHPYYKQFRVAHDLR
VNLEGSRSYQVYCFNLKKAFPLGSDSSVKKWYKKHDGISTKFEDYAMSPR
ITGDELNQKLRAVMYNGHPQNANGIMEGLEPLNAIRVTQEAVWYYSDNAP
ISNPDESFKRESESNLVSTSQLSLMRQALKQLIDPNLATKMPKQVPDDFQ
LSIFESEDKGDKYNKGYQNLLSGGLVPTKPPTPGDPPMPPNQPQTTSVLI
RKYAIGDYSKLLEGATLQLTGDNVNSFQARVFSSNDIGERIELSDGTYTL
TELNSPAGYSIAEPITFKVEAGKVYTIIDGKQIENPNKEIVEPYSVEAYN
DFEEFSVLTTQNYAKFYYAKNKNGSSQVVYCFNADLKSPPDSEDGGKTMT
PDFTTGEVKYTHIAGRDLFKYTVKPRDTDPDTFLKHIKKVIEKGYREKGQ
AIEYSGLTETQLRAATQLAIYYFTDSAELDKDKLKDYHGFGDMNDSTLAV
AKILVEYAQDSNPPQLTDLDFFIPNNNKYQSLIGTQWHPEDLVDIIRMED
KKEVIPVTHNLTLRKTVTGLAGDRTKDFHFEIELKNNKQELLSQTVKTDK
TNLEFKDGKATINLKHGESLTLQGLPEGYSYLVKETDSEGYKVKVNSQEV
ANATVSKTGITSDETLAFENNKEPVVPTGVDQKINGYLALIVIAGISLGI
WGIHTIRIRKHD

GAS 015 contains an amino acid motif indicative of a cell wall anchor: SEQ ID NO:182 VVPTG (shown in italics in SEQ ID NO:48, above). In some recombinant host cell systems, it may be preferable to remove this motif to facilitate secretion of a recombinant GAS 015 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in GAS 015. The pilin motif sequence is underlined in SEQ ID NO:48, below. Conserved lysine (K) residues are also marked in bold, at amino acid residue 243. The pilin sequence, in particular the conserved lysine residues, are thought to be important for the formation of oligomeric, pilus-like structures. Preferred fragments of GAS 015 include the conserved lysine residue. Preferably, fragments include the pilin sequence.

SEQ ID NO: 48

LRGEKMKKTRFPNKLNTLNTQRVLSKNSKRFTVTLVGVFLMIFALVTSMV
GAKTVFGLVESSTPNAINPDSSSEYRWYGYESYVRGHPYYKQFRVAHDLR
VNLEGSRSYQVYCFNLKKAFPLGSDSSVKKWYKKHDGISTKFEDYAMSPR
ITGDELNQKLRAVMYNGHPQNANGIMEGLEPLNAIRVTQEAVWYYSDNAP
ISNPDESFKRESESNLVSTSQLSLMRQALKQLIDPNLATKMPKQVPDDFQ
LSIFESEDKGDKYNKGYQNLLSGGLVPTKPPTPGDPPMPPNQPQTTSVLI
RKYAIGDYSKLLEGATLQLTGDNVNSFQARVFSSNDIGERIELSDGTYTL
TELNSPAGYSIAEPITFKVEAGKVYTIIDGKQIENPNKEIVEPYSVEAYN
DFEEFSVLTTQNYAKFYYAKNKNGSSQVVYCFNADLKSPPDSEDGGKTMT
PDFTTGEVKYTHIAGRDLFKYTVKPRDTDPDTFLKHIKKVIEKGYREKGQ
AIEYSGLTETQLRAATQLAIYYFTDSAELDKDKLKDYHGFGDMNDSTLAV
AKILVEYAQDSNPPQLTDLDFFIPNNNKYQSLIGTQWHPEDLVDIIRMED
KKEVIPVTHNLTLRKTVTGLAGDRTKDFHFEIELKNNKQELLSQTVKTDK
TNLEFKDGKATINLKHGESLTLQGLPEGYSYLVKETDSEGYKVKVNSQEV
ANATVSKTGITSDETLAFENKKEPVVPTGVDQKINGYLALIVIAGISLGI
WGIHTIRIRKHD

An E box containing a conserved glutamic residue has been identified in GAS 015. The E-box motif is underlined in SEQ ID NO:48, below. The conserved glutamic acid (E), at amino acid residue 352, is marked in bold. The E box motif, in particular the conserved glutamic acid residue, is thought to be important for the formation of oligomeric pilus-like structures of GAS 015. Preferred fragments of GAS 015 include the conserved glutamic acid residue. Preferably, fragments include the E box motif.

SEQ ID NO: 48

LRGEKMKKTRFPNKLNTLNTQRVLSKNSKRFTVTLVGVFLMIFALVTSMV
GAKTVFGLVESSTPNAINPDSSSEYRWYGYESYVRGHPYYKQFRVAHDLR
VNLEGSRSYQVYCFNLKKAFPLGSDSSVKKWYKKHDGISTKFEDYAMSPR
ITGDELNQKLRAVMYNGHPQNANGIMEGLEPLNAIRVTQEAVWYYSDNAP
ISNPDESFKRESESNLVSTSQLSLMRQALKQLIDPNLATKMPKQVPDDFQ
LSIFESEDKGDKYNKGYQNLLSGGLVPTKPPTPGDPPMPPNQPQTTSVLI
RKYAIGDYSKLLEGATLQLTGDNVNSFQARVFSSNDIGERIELSDGTYTL
TELNSPAGYSIAEPITFKVEAGKVYTIIDGKQIENPNKEIVEPYSVEAYN
DFEEFSVLTTQNYAKFYYAKNKNGSSQVVYCFNADLKSPPDSEDGGKTMT
PDFTTGEVKYTHIAGRDLFKYTVKPRDTDPDTFLKHIKKVIEKGYREKGQ
AIEYSGLTETQLRAATQLAIYYFTDSAELDKDKLKDYHGFGDMNDSTLAV
AKILVEYAQDSNPPQLTDLDFFIPNNNKYQSLIGTQWHPEDLVDIIRMED
KKEVIPVTHNLTLRKTVTGLAGDRTKDFHFEIELKNNKQELLSQTVKTDK
TNLEFKDGKATINLKHGESLTLQGLPEGYSYLVKETDSEGYKVKVNSQEV
ANATVSKTGITSDETLAFENKKEPVVPTGVDQKINGYLALIVIAGISLGI
WGIHTIRIRKHD

Spy0127 is a LepA putative signal peptidase. An example of an amino acid sequence for Spy0127 is set forth in SEQ ID NO:49.

SEQ ID NO: 49

MIIKRNDMAPSVKAGDAILFYRLSQTYKVEEAVVYEDSKTSITKVGRIIA
QAGDEVDLTEQGELKINGHIQNEGLTFIKSREANYPYRIADNSYLILNDY
YSQESENYLQDAIAKDAIKGTINTLIRLRNH

Spy0128 is thought to be a fimbrial protein. It contains a sortase substrate motif EVXTG (SEQ ID

NO:136) shown in italics in SEQ ID NO:50.

SEQ ID NO: 50

MKLRHLLLTGAALTSFAATTVHGETVVNGAKLTVTKNLDLVNSNALIPNT
DFTFKIEPDTTVNEDGNKFKGVALNTPMTKVTYTNSDKGGSNTKTAEFDF
SEVTFEKPGVYYYKVTEEKIDKVPGVSYDTTSYTVQVHVLWNEEQQKPVA
TYIVGYKEGSKVPIQFKNSLDSTTLTVKKKVSGTGGDRSKDFNFGLTLKA
NQYYKASEKVMIEKTTKGGQAPVQTEASIDQLYHFTLKDGESIKVTNLPV
GVDYVVTEDDYKSEKYTTNVEVSPQDGAVKNIAGNSTEQETSTDKDMTIT
FTNKKDFEVPTGVAMTVAPYIALGIVAVGGALYFVKKKNA

Spy0128 contains an amino acid motif indicative of a cell wall anchor: SEQ ID NO:183 EVPTG (shown in italics in SEQ ID NO:50, above). In some recombinant host cell systems, it may be preferable to remove this motif to facilitate secretion of a recombinant Spy0128 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Two E boxes containing a conserved glutamic residue have been identified in Spy0128. The E-box motifs are underlined in SEQ ID NO:50, below. The conserved glutamic acid (E) residues, at amino acid residues 271 and 290, are marked in bold. The E box motifs, in particular the conserved glutamic acid residues, are thought to be important for the formation of oligomeric pilus-like structures of Spy0128. Preferred fragments of Spy0128 include at least one conserved glutamic acid residue. Preferably, fragments include at least one E box motif.

SEQ ID NO: 50

MKLRHLLLTGAALTSFAATTVHGETVVNGAKLTVTKNLDLVNSNALIPNT
DFTFKIEPDTTVNEDGNKFKGVALNTPMTKVTYTNSDKGGSNTKTAEFDF
SEVTFEKPGVYYYKVTEEKIDKVPGVSYDTTSYTVQVHVLWNEEQQKPVA
TYIVGYKEGSKVPIQFKNSLDSTTLTVKKKVSGTGGDRSKDFNFGLTLKA
NQYYKASEKVMIEKTTKGGQAPVQTEASIDQLYHFTLKDGESIKVTNLPV
GVDYVVTEDDYKSEKYTTNVEVSPQDGAVKNIAGNSTEQETSTDKDMTIT
FTNKKDFEVPTGVAMTVAPYIALGIVAVGGALYFVKKKNA

Spy0129 is a srtC1 type sortase. An example of an amino acid sequence for Spy0129 is set forth in SEQ ID NO:51.

SEQ ID NO: 51

MIVRLIKLLDKLINVIVLCFFFLCLLIAALGIYDALTVYQGANATNYQQY
KKKGVQFDDLLAINSDVMAWLTVKGTHIDIPIVQGENNLEYINKSVEGEY
SLSGSVFLDYRNKVTFEDKYSLIYAHHMAGNVMFGELPNFRKKSFFNKHK
EFSIETKTKQKLKINIFACIQTDAFDSLLFNPIDVDISSKNEFLNHIKQK
SVQYREILTTNESRFVALSTCEDMTTDGRTIVIGQIE”

Spy0130 is referred to as a hypothetical protein. It contains a sortase substrate motif LPXTG (SEQ ID NO:122), shown in italics in SEQ ID NO:52.

SEQ ID NO: 52

MKKSILRILAIGYLLMSFCLLDSVEAENLTASINIEVINQVDVATNKQSS
DIDETFMFVIEALDKESPLPNSVTTSVKGNGKTSFEQLTFSEVGQYHYKI
HQLLGKNSQYHYDETVYEVVIYVLYNEQSGALETNLVSNKLGETEKSELI
FKQEYSEKTPEPHQPDTTEKEKPQKKRNGILPSTGEMVSYVSALGIVLVA
TITLYSIYKKLKTSK

Spy0130 contains an amino acid motif indicative of a cell wall anchor: SEQ ID NO:131 LPSTG (shown in italics in SEQ ID NO:52, above). In some recombinant host cell systems, it may be preferable to remove this motif to facilitate secretion of a recombinant Spy0130 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Two E boxes containing conserved glutamic residues have been identified in Spy0130. The E-box motifs are underlined in SEQ ID NO:52, below. The conserved glutamic acid (E) residues, at amino acid residues 118 and 148, are marked in bold. The E box motifs, in particular the conserved glutamic acid residues, are thought to be important for the formation of oligomeric pilus-like structures of Spy0130. Preferred fragments of Spy0130 include at least one conserved glutamic acid residue. Preferably, fragments include at least one E box motif.

SEQ ID NO: 52

MKKSILRILAIGYLLMSFCLLDSVEAENLTASINIEVINQVDVATNKQSS
DIDETFMFVIEALDKESPLPNSVTTSVKGNGKTSFEQLTFSEVGQYHYKI
HQLLGKNSQYHYDETVYEVVIYVLYNEQSGALETNLVSNKLGETEKSELI
FKQEYSEKTPEPHQPDTTEKEKPQKKRNGILPSTGEMVSYVSALGIVLVA
TITLYSIYKKLKTSK

Spy0131 is referred to as a conserved hypothetical protein. An example of an amino acid sequence of Spy0131 is set forth in SEQ ID NO:53

SEQ ID NO: 53

MTRTNYQKKRMTCPVETEDITYRRKKIKGRRQAILAQFEPELVHHELIGD
SCTCPDCHGTLTEIGSVVQRQELVFIPAQLKRINHVQHAYKCQTCSDNSL
SDKIIKAPVPKAPLAHSLGSASIIAHTVHQKFTLKVPNYRQEEDWNKLGL
SISRKEIANWHIKSSQYYFEPLYDLLRDILLSQEVIHADETSYRVLESDT
QLTYYWTFLSGKHEKKGITLYHHDKRRSGLVTQEVLGDYSGYVHCDMHGA
YRQLEHAKLVGCWAHVRRKFFEATPKQADKTSLGRKGLVYCDKLFALEAE
WCELPPQERLVKRKEILTPLMTTFFDWCREQVVLSGSKLGLAIAYSLKHE
RTFRTVLEDGHIVLSNNMAERAIKSLVMGRKNWLFSQSFEGAKAAAIIMS
LLETAKRHGLNSEKYISYLLDRLPNEETLAKREVLEAYLPWAKKVQTNCQ

Spy0133 is referred to as a conserved hypothetical protein. An example of an amino acid sequence of Spy0133 is set forth in SEQ ID NO:54.

SEQ ID NO: 54

MTIRLNDLGQVYLVCGKTDMRQGIDSLAYLVKSQHELDLFSGAVYLFCGG
RRDRFKALYWDGQGFWLLYKRFENGKLAWPRNRDEVKCLTAVQVDWLMKG
FFISPNIKISKSHDFY

Spy0135 is a SrtB type sortase. It is also referred to as a putative fimbria-associated protein. An example of an amino acid sequence of Spy0135 is set forth in SEQ ID NO:55.

SEQ ID NO: 55

MECYRDRQLLSTYHKQVTQKKPSEMEEVWQKAKAYNARLGIQPVPDAFSF
RDGIHDKNYESLLQIENNDIMGYVEVPSIKVTLPIYHYTTDEVLTKGAGH
LFGSALPVGGDGTHTVISAHRGLPSAEMFTNLNLVKKGDTFYFRVLNKVL
AYKVDQILTVEPDQVTSLSGVMGKDYATLVTCTPYGVNTKRLLVRGHRIA
YHYKKYQQAKKAMKLVDKSRMWAEVVCAAFGVVIAIILVFMYSRVSAKKS
K

GAS AI-3 Sequences

As discussed above, a GAS AI-3 sequence is present in a M3, M18 and M5 strain isolates. Examples of GAS AI-3 sequences from M3 strain isolate MGAS315 are set forth below.

SpyM30097 is as a negative transcriptional regulator (Nra). An example of an amino acid sequence of SpyM30097 is set forth in SEQ ID NO:56.

SEQ ID NO: 56

MPYVKKKKDSFLVETYLEQSIRDKSELVLLLFKSPTIIFSHVAKQTGLTA
VQLKYYCKELDDFFGNNLDITIKKGKIICCFVKPVKEFYLHQLYDTSTIL
KLLVFFIKNGTSSQPLIKFSKKYFLSSSSAYRLRESLIKLLREFGLRVSK
NTIVGEEYRIRYLIAMLYSKFGIVIYPLDHLDNQIIYRFLSQSATNLRTS
PWLEEPFSFYNMLLALSWKRHQFAVSIPQTRIFRQLKKLFIYDCLTRSSR
QVIENAFSLTFSQGDLEYLFLIYITTNNSFASLQWTPQHIETCCHIFEKN
DTFRLLLEPILKRLPQLNHSKQDLIKALMYFSKSFLFNLQHFVIEIPSFS
LPTYTGNSNLYKALKNIVNQWLAQLPGKRHLNEKHLQLFCSHIEQILKNK
QPALTVVLISSNFINAKLLTDTIPRYFSDKGIHFYSFYLLRDDIYQIPSL
KPDLVITHSRLIPFVKNDLVKGVTVAEFSFDNPDYSIASIQNLIYQLKDK
KYQDFLNEQLQ

SpyM30098 is thought to be a collagen binding protein (Cpb). It contains a sortase substrate motif VPXTG (SEQ ID NO:137) shown in italics in SEQ ID NO:57.

SEQ ID NO: 57

MQKRDKTNYGSANNKRRQTTIGLLKVFLTFVALIGIVGFSIRAFGAEEQS
VPNKQSSVQDYPWYGYDSYSKGYPDYSPLKTYHNLKVNLDGSKEYQAYCF
NLTKHFPSKSDSVRSQWYKKLEGTNENFIKLADKPRIEDGQLQQNILRIL
YNGYPNDRNGIMKGIDPLNAILVTQNAIWYYTDSSYISDTSKAFQQEETD
LKLDSQQLQLMRNALKRLINPKEVESLPNQVPANYQLSIFQSSDKTFQNL
LSAEYVPDTPPKPGEEPPAKTEKTSVIIRKYAEGDYSKLLEGATLKLAQI
EGSGFQEKIFDSNKSGEKVELPNGTYVLSELKPPQGYGVATPITFKVAAE
KVLIKNKEGQFVENQNKEIAEPYSVTAFNDFEEIGYLSDFNNYGKFYYAK
NTNGTNQVVYCFNADLHSPPDSYDHGANIDPDVSESKEIKYTHVSGYDLY
KYAATPRDKDADFFLKHIKKILDKGYKKKGDTYKTLTEAQFRAATQLAIY
YYTDSADLTTLKTYNDNKGYHGFDKLDDATLAVVHELITYAEDVTLPMTQ
NLDFFVPNSSRYQALIGTQYHPNELIDVISMEDKQAPIIPITHKLTISKT
VTGTIADKKKEFNFEIHLKSSDGQAISGTYPTNSGELTVTDGKATFTLKD
GESLIVEGLPSGYSYEITETGASDYEVSVNGKNAPDGKATKASVKEDETV
AFENRKDLVPPTGLTTDGAIYLWLLLLVPFGLLVWLFGRKGTKK

SpyM30098 contains an amino acid motif indicative of a cell wall anchor: SEQ ID NO:184 VPPTG (shown in italics in SEQ ID NO:57, above). In some recombinant host cell systems, it may be preferable to remove this motif to facilitate secretion of a recombinant SpyM30098 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in SpyM30098. The pilin motif sequence is underlined in SEQ ID NO:57, below. Conserved lysine (K) residues are also marked in bold, at amino acid residues 262 and 270. The pilin sequence, in particular the conserved lysine residues, are thought to be important for the formation of oligomeric, pilus-like structures. Preferred fragments of SpyM30098 include at least one conserved lysine residue. Preferably, fragments include the pilin sequence.

SEQ ID NO: 57

MQKRDKTNYGSANNKRRQTTIGLLKVFLTFVALIGIVGFSIRAFGAEEQS
VPNKQSSVQDYPWYGYDSYSKGYPDYSPLKTYHNLKVNLDGSKEYQAYCF
NLTKHFPSKSDSVRSQWYKKLEGTNENFIKLADKPRIEDGQLQQNILRIL
YNGYPNDRNGIMKGIDPLNAILVTQNAIWYYTDSSYISDTSKAFQQEETD
LKLDSQQLQLMRNALKRLINPKEVESLPNQVPANYQLSIFQSSDKTFQNL
LSAEYVPDTPPKPGEEPPAKTEKTSVIIRKYAEGDYSKLLEGATLKLAQI
EGSGFQEKIFDSNKSGEKVELPNGTYVLSELKPPQGYGVATPITFKVAAE
KVLIKNKEGQFVENQNKEIAEPYSVTAFNDFEEIGYLSDFNNYGKFYYAK
NTNGTNQVVYCFNADLHSPPDSYDHGANIDPDVSESKEIKYTHVSGYDLY
KYAATPRDKDADFFLKHIKKILDKGYKKKGDTYKTLTEAQFRAATQLAIY
YYTDSADLTTLKTYNDNKGYHGFDKLDDATLAVVHELITYAEDVTLPMTQ
NLDFFVPNSSRYQALIGTQYHPNELIDVISMEDKQAPIIPITHKLTISKT
VTGTIADKKKEFNFEIHLKSSDGQAISGTYPTNSGELTVTDGKATFTLKD
GESLIVEGLPSGYSYEITETGASDYEVSVNGKNAPDGKATKASVKEDETV
AFENRKDLVPPTGLTTDGAIYLWLLLLVPFGLLVWLFGRKGTKK

An E box containing a conserved glutamic residue has been identified in SpyM30098. The E-box motif is underlined in SEQ ID NO:57, below. The conserved glutamic acid (E), at amino acid residue 330, is marked in bold. The E box motif, in particular the conserved glutamic acid residue, is thought to be important for the formation of oligomeric pilus-like structures of SpyM30098. Preferred fragments of SpyM30098 include the conserved glutamic acid residue. Preferably, fragments include the E box motif.

SEQ ID NO: 57

MQKRDKTNYGSANNKRRQTTIGLLKVFLTFVALIGIVGFSIRAFGAEEQS
VPNKQSSVQDYPWYGYDSYSKGYPDYSPLKTYHNLKVNLDGSKEYQAYCF
NLTKHFPSKSDSVRSQWYKKLEGTNENFIKLADKPRIEDGQLQQNILRIL
YNGYPNDRNGIMKGIDPLNAILVTQNAIWYYTDSSYISDTSKAFQQEETD
LKLDSQQLQLMRNALKRLINPKEVESLPNQVPANYQLSIFQSSDKTFQNL
LSAEYVPDTPPKPGEEPPAKTEKTSVIIRKYAEGDYSKLLEGATLKLAQI
EGSGFQEKIFDSNKSGEKVELPNGTYVLSELKPPQGYGVATPITFKVAAE
KVLIKNKEGQFVENQNKEIAEPYSVTAFNDFEEIGYLSDFNNYGKFYYAK
NTNGTNQVVYCFNADLHSPPDSYDHGANIDPDVSESKEIKYTHVSGYDLY
KYAATPRDKDADFFLKHIKKILDKGYKKKGDTYKTLTEAQFRAATQLAIY
YYTDSADLTTLKTYNDNKGYHGFDKLDDATLAVVHELITYAEDVTLPMTQ
NLDFFVPNSSRYQALIGTQYHPNELIDVISMEDKQAPIIPITHKLTISKT
VTGTIADKKKEFNFEIHLKSSDGQAISGTYPTNSGELTVTDGKATFTLKD
GESLIVEGLPSGYSYEITETGASDYEVSVNGKNAPDGKATKASVKEDETV
AFENRKDLVPPTGLTTDGAIYLWLLLLVPFGLLVWLFGRKGTKK

SpyM30099 is referred to as LepA. An example of an amino acid sequence of SpyM30099 is set forth in SEQ ID NO:58.

SEQ ID NO: 58

MTNYLNRLNENPLLKAFIRLVLKISIIGFLGYILFQYVFGVMIVNTNQMS
PAVSAGDGVLYYRLTDRYHINDVVVYEVDDTLKVGRIAAQAGDEVNFTQE
GGLLINGHPPEKEVPYLTYPHSSGPNFPYKVPTGTYFTLNDYREERLDSR
YYGALPINQIKGKISTLLRVRGI

SpyM30100 is thought to be a fimbrial protein. An example of an amino acid sequence of SpyM30100 is set forth in SEQ ID NO:59.

SEQ ID NO: 59

MKKNKLLLATAILATALGTASLNQNVKAETAGVSENAKLIVKKTFDSYTD
NEVLMPKADYTFKVEADSTASGKTKDGLEIKPGIVNGLTEQIISYTNTDK
PDSKVKSTEFDFSKVVFPGIGVYRYTVSEKQGDVEGITYDTKKWTVDVYV
GNKEGGGFEPKFIVSKEQGTDVKKPVNFNNSFATTSLKVKKNVSGNTGEL
QKEFDFTLTLNESTNFKKDQIVSLQKGNEKFEVKIGTPYKFKLKNGESIQ
LDKLPVGITYKVNEMEANKDGYKTTASLKEGDGQSKMYQLDMEQKTDESA
DEIVVTNKRDTQVPTGVVGTLAPFAVLSIVAIGGVIYITKRKKA

SpyM30100 contains an amino acid motif indicative of a cell wall anchor: SEQ ID NO:140 QVPTG (shown in italics in SEQ ID NO:59, above). In some recombinant host cell systems, it may be preferable to remove this motif to facilitate secretion of a recombinant SpyM30100 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Two pilin motifs, discussed above, containing conserved lysine (K) residues have also been identified in SpyM30100. The pilin motif sequences are underlined in SEQ ID NO:59, below. Conserved lysine (K) residues are also marked in bold, at amino acid residues 57 and 63 and at amino acid residues 161 and 166. The pilin sequences, in particular the conserved lysine residues, are thought to be important for the formation of oligomeric, pilus-like structures. Preferred fragments of SpyM30100 include at least one conserved lysine residue. Preferably, fragments include at least one pilin sequence.

SEQ ID NO: 59

MKKNKLLLATAILATALGTASLNQNVKAETAGVSENAKLIVKKTFDSYTD
NEVLMPKADYTFKVEADSTASGKTKDGLEIKPGIVNGLTEQIISYTNTDK
PDSKVKSTEFDFSKVVFPGIGVYRYTVSEKQGDVEGITYDTKKWTVDVYV
GNKEGGGFEPKFIVSKEQGTDVKKPVNFNNSFATTSLKVKKNVSGNTGEL
QKEFDFTLTLNESTNFKKDQIVSLQKGNEKFEVKIGTPYKFKLKNGESIQ
LDKLPVGITYKVNEMEANKDGYKTTASLKEGDGQSKMYQLDMEQKTDESA
DEIVVTNKRDTQVPTGVVGTLAPFAVLSIVAIGGVIYITKRKKA

Two E boxes, each containing a conserved glutamic residue, have been identified in SpyM30100. The E-box motifs are underlined in SEQ ID NO:59, below. The conserved glutamic acid (E) residues, at amino acid residues 232 and 264, are marked in bold. The E box motifs, in particular the conserved glutamic acid residues, are thought to be important for the formation of oligomeric pilus-like structures of SpyM30100. Preferred fragments of SpyM30100 include at least one conserved glutamic acid residue. Preferably, fragments include at least one E box motif.

SEQ ID NO: 59

MKKNKLLLATAILATALGTASLNQNVKAETAGVSENAKLIVKKTFDSYTD
NEVLMPKADYTFKVEADSTASGKTKDGLEIKPGIVNGLTEQIISYTNTDK
PDSKVKSTEFDFSKVVFPGTGVYRYTVSEKQGDVEGITYDTKKWTVDVYV
GNKEGGGFEPKFIVSKEQGTDVKKPVNFNNSFATTSLKVKKNVSGNTGEL
QKEFDFTLTLNESTNFKKDQIVSLQKGNEKFEVKIGTPYKFKLKNGESIQ
LDKLPVGITYKVNEMEANKDGYKTTASLKEGDGQSKMYQLDMEQKTDESA
DEIVVTNKRDTQVPTGVVGTLAPFAVLSIVAIGGVIYITKRKKA

SpyM30101 is a SrtC2 type sortase. An example of an amino acid sequence of SpyM30101 is set forth in SEQ ID NO:60.

SEQ ID NO: 60

MTIVQVINKAIDTLILIFCLVVLFLAGFGLWDSYHLYQQADASNFKKFKT
AQQQPKFEDLLALNEDVIGWLNIPGTHIDYPLVQGKTNLEYINKAVDGSV
AMSGSLFLDTRNHNDFTDDYSLIYGHHMAGNAMFGEIPKFLKKDFFSKHN
KAIIETKERKKLTVTIFACLKTDAFNQLVFNPNAITNQDQQRQLVDYISK
RSKQFKPVKLKHHTKFVAFSTCENFSTDNRVIVVGTIQE

SpyM30102 is referred to as a hypothetical protein. An example of an amino acid sequence of SpyM30102 is set forth in SEQ ID NO:61.

SEQ ID NO: 61

MILTMLAFNQTVLAKDSTVQTSISVENVLERAGDSTPFSIALESIDAMKT
IEEITIAGSGKASFSPLTFTTVGQYTYRVYQKPSQNKDYQADTTVFDVLV
YVTYDEDGTLVAKVISRRAGDEEKSAITFKPKWLVKPIPPRQPNIPKTPL
PLAGEVKSLLGILSIVLLGLLVLLYVKKLKSRL

SpyM30102 contains an amino acid motif indicative of a cell wall anchor: SEQ ID NO:185 LPLAG (shown in italics in SEQ ID NO:61, above). In some recombinant host cell systems, it may be preferable to remove this motif to facilitate secretion of a recombinant SpyM30102 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in SpyM30102. The pilin motif sequence is underlined in SEQ ID NO:61, below. The conserved lysine (K) residue is also marked in bold, at amino acid residue 132. The pilin sequence, in particular the conserved lysine residues, are thought to be important for the formation of oligomeric, pilus-like structures. Preferred fragments of SpyM30102 include the conserved lysine residue. Preferably, fragments include the pilin sequence.

SEQ ID NO: 61

MILTMLAFNQTVLAKDSTVQTSISVENVLERAGDSTPFSIALESIDAMKT
IEEITIAGSGKASFSPLTFTTVGQYTYRVYQKPSQNKDYQADTTVFDVLV
YVTYDEDGTLVAKVISRRAGDEEKSAITFKPKWLVKPIPPRQPNIPKTPL
PLAGEVKSLLGILSIVLLGLLVLLYVKKLKSRL

Two E boxes containing conserved glutamic residues have been identified in SpyM30102. The E-box motifs are underlined in SEQ ID NO:61, below. The conserved glutamic acid (E) residues, at amino acid residues 52 and 122, are marked in bold. The E box motifs, in particular the conserved glutamic acid residues, are thought to be important for the formation of oligomeric pilus-like structures of SpyM30102. Preferred fragments of SpyM30102 include at least one conserved lysine residue. Preferably, fragments include at least one pilin sequence.

SEQ ID NO: 61

MILTMLAFNQTVLAKDSTVQTSISVENVLERAGDSTPFSIALESIDAMKT
IEEITIAGSGKASFSPLTFTTVGQYTYRVYQKPSQNKDYQADTTVFDVLV
YVTYDEDGTLVAKVISRRAGDEEKSAITFKPKWLVKPIPPRQPNIPKTPL
PLAGEVKSLLGILSIVLLGLLVLLYVKKLKSRL

SpyM30103 is referred to as a putative multiple sugar metabolism regulator. An example of an amino acid sequence for SpyM3103 is set forth in SEQ ID NO:62.

SEQ ID NO: 62

MVRFDLKHVQTLHSLSQLPISVMSQDKALIQVYGNDDYLLCYYQFLKHLA
IPQAAQDVIFYEGLFEESFMIFPLCHYIIAIGPFYPYSLNKDYQEQLANN
CLKHSSHRSKEELLSYMALVPHFPINNVRNLLIAIDAFFDTQFETTCQQT
IHQLLQHSKQMTADPDIIHRLKHISKASSQLPPVLEHLNHIMDLVKLGNP
QLLKQEINRIPLSSITSSSISALRAEKNLTVIYLTRLLEFSFVENTDVAK
HYSLVKYYMALNEEASDLLKVLRIRCAAIIHFSESLTNKSISDKRQMYNS
VLHYVDSHLYSKLKVSDIAKRLYVSESHLRSVFKKYSNVSLQHYILSTKI
KEAQLLLKRGIPVGEVAKSLYFYDTTHFHKIFKKYTGISSKDYLAKYRDN
I

SpyM30104 is thought to be a F2 like fibronectin binding protein. An example of an amino acid sequence for SpyM30104 is set forth in SEQ ID NO:63.

SEQ ID NO: 63

MSSSDEETLKQYASKYTSNRRGDTSGNLKKQIAKVLTEGYPTNKSDWLNG
LTENEKIEVTQDAIWYFTETTVPADRSYTNRNVNSQKMKEVYQKLIDTTD
IDKYEDVQFDLFVPQDTNLQAVISVEPVIESLPWTSLKPIAQKDITAKKI
WVDAPKEKPIIYFKLYRQLPGEKEVAVDDAELKQINSEGQQEISVTWTNQ
LVTDEKGMAYIYSVKEVDKNGELLEPKDYIKKEDGLTVTNTYVKPTSGHY
DIEVTFGNGHIDITEDTTPDIVSGENQMKQIEGEDSKPIDEVTENNLIEF
GKNTMPGEEDGTNSNKYEEVEDSRPVDTLSGLSSEQGQSGDMTIEEDSAT
HIKFSKRDIDGKELAGATMELRDSSGKTISTWISDGQVKDFYLMPGKYTF
VETAAPDGYEVATAITFTVNEQGQVTVNGKATKGDAHIVMVDAYKPTKGS
GQVIDIEEKLPDEQGHSGSTTEIEDSKSSDVIIGGQGEVVDTTEDTQSGM
TGHSGSTTEIEDSKSSDVIIGGQGEVVDTTEDTQSGMTGHSGSTTKIEDS
KSSDVIVGGQGQIVETTEDTQTGMHGDSGRKTEVEDTKLVQSFHFDNKEP
ESNSEIPKKDKSKSNTSLPATGEKQHNKFFWMVTSCSLISSVFVISLKSK
KRLSSC

SpyM30104 contains an amino acid motif indicative of a cell wall anchor: SEQ ID NO:180 LPATG (shown in italics in SEQ ID NO:63, above). In some recombinant host cell systems, it may be preferable to remove this motif to facilitate secretion of a recombinant SpyM30104 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Two pilin motifs, discussed above, containing conserved lysine (K) residues have also been identified in SpyM30104. The pilin motif sequences are underlined in SEQ ID NO:63, below. Conserved lysine (K) residues are also marked in bold, at amino acid residues 156 and 227. The pilin sequences, in particular the conserved lysine residues, are thought to be important for the formation of oligomeric, pilus-like structures. Preferred fragments of SpyM30104 include at least one conserved lysine residue. Preferably, fragments include at least one pilin sequence.

SEQ ID NO: 63

MSSSDEETLKQYASKYTSNRRGDTSGNLKKQIAKVLTEGYPTNKSDWLNG
LTENEKIEVTQDAIWYFTETTVPADRSYTNRNVNSQKMKEVYQKLIDTTD
IDKYEDVQFDLFVPQDTNLQAVISVEPVIESLPWTSLKPIAQKDITAKKI
WVDAPKEKPIIYFKLYRQLPGEKEVAVDDAELKQINSEGQQEISVTWTNQ
LVTDEKGMAYIYSVKEVDKNGELLEPKDYIKKEDGLTVTNTYVKPTSGHY
DIEVTFGNGHIDITEDTTPDIVSGENQMKQIEGEDSKPIDEVTENNLIEF
GKNTMPGEEDGTNSNKYEEVEDSRPVDTLSGLSSEQGQSGDMTIEEDSAT
HIKFSKRDIDGKELAGATMELRDSSGKTISTWISDGQVKDFYLMPGKYTF
VETAAPDGYEVATAITFTVNEQGQVTVNGKATKGDAHIVMVDAYKPTKGS
GQVIDIEEKLPDEQGHSGSTTEIEDSKSSDVIIGGQGEVVDTTEDTQSGM
TGHSGSTTEIEDSKSSDVIIGGQGEVVDTTEDTQSGMTGHSGSTTKIEDS
KSSDVIVGGQGQIVETTEDTQTGMHGDSGRKTEVEDTKLVQSFHFDNKEP
ESNSEIPKKDKSKSNTSLPATGEKQHNKFFWMVTSCSLISSVFVISLKSK
KRLSSC

An E box containing a conserved glutamic residue has been identified in SpyM30104. The E-box motif is underlined in SEQ ID NO:63, below. The conserved glutamic acid (E), at amino acid residue 402, is marked in bold. The E box motif, in particular the conserved glutamic acid residue, is thought to be important for the formation of oligomeric pilus-like structures of SpyM30104. Preferred fragments of SpyM30104 include the conserved glutamic acid residue. Preferably, fragments include the E box motif.

SEQ ID NO: 63

MSSSDEETLKQYASKYTSNRRGDTSGNLKKQIAKVLTEGYPTNKSDWLNG
LTENEKIEVTQDAIWYFTETTVPADRSYTNRNVNSQKMKEVYQKLIDTTD
IDKYEDVQFDLFVPQDTNLQAVISVEPVIESLPWTSLKPIAQKDITAKKI
WVDAPKEKPIIYFKLYRQLPGEKEVAVDDAELKQINSEGQQEISVTWTNQ
LVTDEKGMAYIYSVKEVDKNGELLEPKDYIKKEDGLTVTNTYVKPTSGHY
DIEVTFGNGHIDITEDTTPDIVSGENQMKQIEGEDSKPIDEVTENNLIEF
GKNTMPGEEDGTNSNKYEEVEDSRPVDTLSGLSSEQGQSGDMTIEEDSAT
HIKFSKRDIDGKELAGATMELRDSSGKTISTWISDGQVKDFYLMPGKYTF
VETAAPDGYEVATAITFTVNEQGQVTVNGKATKGDAHIVMVDAYKPTKGS
GQVIDIEEKLPDEQGHSGSTTEIEDSKSSDVIIGGQGEVVDTTEDTQSGM
TGHSGSTTEIEDSKSSDVIIGGQGEVVDTTEDTQSGMTGHSGSTTKIEDS
KSSDVIVGGQGQIVETTEDTQTGMHGDSGRKTEVEDTKLVQSFHFDNKEP
ESNSEIPKKDKSKSNTSLPATGEKQHNKFFWMVTSCSLISSVFVISLKSK
KRLSSC

Examples of GAS AI-3 sequences from M3 strain isolate SSI-1 are set forth below.

Sps0099 is a negative transcriptional regulator (Nra). An example of an amino acid sequence for Sps0099 is set forth in SEQ ID NO:64.

SEQ ID NO: 64

MPYVKKKKDSFLVETYLEQSIRDKSELVLLLFKSPTIIFSHVAKQTGLTA
VQLKYYCKELDDFFGNNLDITIKKGKIICCFVKPVKEFYLHQLYDTSTIL
KLLVFFIKNGTSSQPLIKFSKKYFLSSSSAYRLRESLIKLLREFGLRVSK
NTIVGEEYRIRYLIAMLYSKFGIVIYPLDHLDNQIIYRFLSQSATNLRTS
PWLEEPFSFYNMLLALSWKRHQFAVSIPQTRIFRQLKKLFIYDCLTRSSR
QVIENAFSLTFSQGDLEYLFLIYITTNNSFASLQWTPQHIETCCHIFEKN
DTFRLLLEPILKRLPQLNHSKQDLIKALMYFSKSFLFNLQHFVIEIPSFS
LPTYTGNSNLYKALKNIVNQWLAQLPGKRHLNEKHLQLFCSHIEQILKNK
QPALTVVLISSNFINAKLLTDTIPRYFSDKGIHFYSFYLLRDDIYQIPSL
KPDLVITHSRLIPFVKNDLVKGVTVAEFSFDNPDYSIASIQNLIYQLKDK
KYQDFLNEQLQ

Sps0100 is thought to be a collagen binding protein (Cbp). It contains a sortase substrate motif VPXTG shown in italics in SEQ ID NO:65.

SEQ ID NO: 65

MQKRDKTNYGSANNKRRQTTIGLLKVFLTFVALIGIVGFSIRAFGAEEQS
VPNKQSSVQDYPWYGYDSYSKGYPDYSPLKTYHNLKVNLDGSKEYQAYCF
NLTKHFPSKSDSVRSQWYKKLEGTNENFIKLADKPRIEDGQLQQNILRIL
YNGYPNDRNGIMKGIDPLNAILVTQNAIWYYTDSSYISDTSKAFQQEETD
LKLDSQQLQLMRNALKRLINPKEVESLPNQVPANYQLSIFQSSDKTFQNL
LSAEYVPDTPPKPGEEPPAKTEKTSVIIRKYAEGDYSKLLEGATLKLAQI
EGSGFQEKIFDSNKSGEKVELPNGTYVLSELKPPQGYGVATPITFKVAAE
KVLIKNKEGQFVENQNKEIAEPYSVTAFNDFEEIGYLSDFNNYGKFYYAK
NTNGTNQVVYCFNADLHSPPDSYDHGANIDPDVSESKEIKYTHVSGYDLY
KYAATPRDKDADFFLKHIKKILDKGYKKKGDTYKTLTEAQFRAATQLAIY
YYTDSADLTTLKTYNDNKGYHGFDKLDDATLAVVHELITYAEDVTLPMTQ
NLDFFVPNSSRYQALIGTQYHPNELIDVISMEDKQAPIIPITHKLTISKT
VTGTIADKKKEFNFEIHLKSSDGQAISGTYPTNSGELTVTDGKATFTLKD
GESLIVEGLPSGYSYEITETGASDYEVSVNGKNAPDGKATKASVKEDETV
AFENRKDLVPPTGLTTDGAIYLWLLLLVPFGLLVWLFGRKGTKK

Sps0101 is referred to as a LepA protein. An example of an amino acid sequence of Sps0101 is set forth as SEQ ID NO: 66

SEQ ID NO: 66

MTNYLNRLNENPLLKAFIRLVLKISIIGFLGYILFQYVFGVMIVNTNQMS
PAVSAGDGVLYYRLTDRYHINDVVVYEVDDTLKVGRIAAQAGDEVNFTQE
GGLLINGHPPEKEVPYLTYPHSSGPNFPYKVPTGTYFILNDYREERLDSR
YYGALPINQIKGKISTLLRVRGI

Sps0102 is thought to be a fimbrial protein. It contains a sortase substrate motif QVXTG shown in italics in SEQ ID NO:67.

SEQ ID NO: 67

MEREKMKKNKLLLATAILATALGTASLNQNVKAETAGVSENAKLIVKKTF
DSYTDNEVLMPKADYTFKVEADSTASGKTKDGLEIKPGIVNGLTEQIISY
TNTDKPDSKVKSTEFDFSKVVFPGIGVYRYTVSEKQGDVEGITYDTKKWT
VDVYVGNKEGGGFEPKFIVSKEQGTDVKKPVNFNNSFATTSLKVKKNVSG
NTGELQKEFDFTLTLNESTNFKKDQIVSLQKGNEKFEVKIGTPYKFKLKN
GESIQLDKLPVGITYKVNEMEANKDGYKTTASLKEGDGQSKMYQLDMEQK
TDESADEIVVTNKRDTQVPTGVVGTLAPFAVLSIVAIGGVIYITKRKKA

Sps0103 is a SrtC2 type sortase. An example of Sps0103 is set forth in SEQ ID NO:68.

SEQ ID NO: 68

MVMTIVQVINKAIDTLILIFCLVVLFLAGFGLWDSYHLYQQADASNFKKF
KTAQQQPKFEDLLALNEDVIGWLNIPGTHIDYPLVQGKTNLEYINKAVDG
SVAMSGSLFLDTRNHNDFTDDYSLIYGHHMAGNAMFGEIPKFLKKDFFSK
HNKAIIETKERKKLTVTIFACLKTDAFNQLVFNPNAITNQDQQRQLVDYI
SKRSKQFKPVKLKHHTKFVAFSTCENFSTDNRVIVVGTIQE

Sps0104 is referred to as a hypothetical protein. It contains a sortase substrate motif LPXAG shown in italics in SEQ ID NO:69.

SEQ ID NO: 69

MLFSVVMILTMLAFNQTVLAKDSTVQTSISVENVLEPAGDSTPFSIALES
IDAMKTIEEITIAGSGKASFSPLTFTTVGQYTYRVYQKPSQNKDYQADTT
VFDVLVYVTYDEDGTLVAKVISRRAGDEEKSAITFKPKWLVKPIPPRQPN
IPKTPLPLAGEVKSLLGILSIVLLGLLVLLYVKKLKSRL

Sps0105 is referred to as a putative multiple sugar metabolism regulator. An example of Sps0105 is set forth in SEQ ID NO:70.

SEQ ID NO: 70

MALVPHFPINNVRNLLIAIDAFFDTQFETTCQQTIHQLLQHSKQMTADPD
IIHRLKHISKASSQLPPVLEHLNHIMDLVKLGNPQLLKQEINRIPLSSIT
SSSISALRAEKNLTVIYLTRLLEFSFVENTDVAKHYSLVKYYMALNEEAS
DLLKVLRIRCAAIIHFSESLTNKSISDKRQMYNSVLHYVDSHLYSKLKVS
DIAKRLYVSESHLRSVFKKYSNVSLQHYILSTKIKEAQLLLKRGIPVGEV
AKSLYFYDTTHFHKIFKKYTGISSKDYLAKYRDNI

Sps0106 is thought to be a F2 like fibronectin binding protein. It contains a sortase substrate LPXTG (SEQ ID NO:122) shown in italics in SEQ ID NO:71.

SEQ ID NO: 71

MTQKNSYKLSFLLSLTGFILGLLLVFIGLSGVSVGHAETRNGANKQGAFE
IKKNKSQEEYNYEVYDNRNILQDGEHKLEIKRVDGTGKTYQGFCFQLTKN
FPTAQGVSKKLYKKLSSSDEETLKQYASKYTSNRRGDTSGNLKKQIAKVL
TEGYPTNKSDWLNGLTENEKIEVTQDAIWYFTETTVPADRSYTNRNVNSQ
KMKEVYQKLIDTTDIDKYEDVQFDLFVPQDTNLQAVISVEPVIESLPWTS
LKPIAQKDITAKKIWVDAPKEKPIIYFKLYRQLPGEKEVAVDDAELKQIN
SEGQQEISVTWTNQLVTDEKOMAYIYSVKEVDKNGELLEPKDYIKKEDGL
TVTNTYVKPTSGHYDIEVTFGNGHIDITEDTTPDIVSGENQMKQIEGEDS
KPIDEVTENNLIEFGKNTMPGEEDGTNSNKYEEVEDSRPVDTLSGLSSEQ
GQSGDMTIEEDSATHIKFSKRDIDGKELAGATMELRDSSGKTISTWISDG
QVKDFYLMPGKYTFVETAAPDGYEVATAITFTVNEQGQVTVNGKATKGDA
HIVMVDAYKPTKGSGQVIDIEEKLPDEQGHSGSTTEIEDSKSSDVIIGGQ
GEVVDTTEDTQSGMTGHSGSTTKIEDSKSSDVIVGGQGQIVETTEDTQTG
MHGDSGRKTEVEDTKLVQSFHFDNKEPESNSEIPKKDKSKSNTSLPATGE
KQHNKFFWMVTSCSLISSVFVISLKSKKRLSSC

Examples of GAS AI-3 sequences from M5 isolate Manfredo are set forth below.

Orf 77 encodes a negative transcription regulator (Nra). An example of the nucleotide sequence encoding Nra (SEQ ID NO:88) and an Nra amino acid sequence (SEQ ID NO:89) are set forth below.

SEQ ID NO: 88

ATGCCTTATGTCAAAAAGAAAAAGGATAGTTTCTTAGTAGAAACATATCT
TGAACAGTCTATTAGAGATAAAAGTGAATTAGTCTTACTGTTATTTAAAT
CGCCTACTATCATTTTTTCTCATGTTGCTAAACAAACTGGTCTGACGGCT
GTACAATTAAAATATTACTGTAAAGAACTTGATGACTTTTTTGGAAATAA
TTTAGACATTACCATTAAAAAGGGCAAAATAATATGTTGTTTTGTCAAAC
CTGTTAAGGAATTCTACCTTCATCAACTCTATGACACATCAACAATATTA
AAATTATTAGTTTTCTTTATTAAAAATGGAACGTCATCACAACCTCTGAT
TAAATTTTCAAAAAAGTATTTTCTATCAAGCTCCTCAGCTTATCGACTAC
GGGAATCGCTGATCAAATTACTACGGGAATTTGGCTTGAGAGTCTCAAAA
AATACAATTGTCGGAGAGGAATATCGTATTCGCTATCTTATTGCCATGCT
ATATAGTAAATTTGGCATTGTCATCTATCCGTTAGATCATCTAGACAATC
AAATTATTTATCGCTTCTTATCACAAAGTGCAACCAATTTAAGAACATCG
CCCTGGCTAGAGGAACCTTTTTCTTTTTATAATATGTTACTTGCCTTGTC
ATGGAAACGTCACCAATTTGCAGTTAGCATTCCTCAAACACGTATTTTTC
GACAATTAAAAAAGCTTTTTATCTATGATTGTTTAACTCGAAGCAGTCGA
CAAGTAATCGAAAATGCTTTTTCGTTAATGTTCTCACAAGGAGATCTCGA
TTATCTTTTTTTAATTTATATTACCACCAATAATTCCTTTGCCAGCCTAC
AATGGACTCCACAGCATATTGAAACTTGCTGCCATATTTTTGAAAAAAAT
GACACATTTCGGTTATTGTTAGAGCCCATTCTTAAACGTTTACCGCAATT
AAACCATTCTAAACAAGACCTTATTAAAGCCCTTATGTATTTTTCAAAAT
CTTTTCTATTTAACCTCCAACATTTCGTCATCGAGATTCCTTCTTTTTCC
TTGCCGACCTATACAGGCAACTCTAATCTTTACAAAGCTTTAAAAAATAT
TGTAAATCAGTGGCTTGCTCAATTACCCGGAAAGCGTCATCTTAACGAAA
AGCATCTCCAACTTTTTTGCTCTCATATTGAACAAATCTTAAAAAATAAA
CAACCTGCTTTAACTGTCGTTTTAATATCTAGTAACTTTATAAATGCTAA
ACTCCTTACAGATACTATCCCACGATATTTTTCTGATAAAGGAATTCATT
TTTATTCTTTTTACTTATTAAGAGATGATATCTATCAAATTCCAAGCTTA
AAACCAGATTTAGTTATCACTCATAGCCGATTAATTCCTTTTGTTAAGAA
TGATCTGGTCAAAGGTGTTACTGTTGCTGAATTTTCTTTTGATAACCCTG
ACTACTCTATTGCTTCAATTCAAAACTTGATATATCAGCTCAAAGATAAA
AAATATCAAGATTTTCTAAACGAGCAATTACAA

SEQ ID NO: 89

MPYVKKKKDSFLVETYLEQSIRDKSELVLLLFKSPTIIFSHVAKQTGLTA
VQLKYYCKELDDFFGNNLDITIKKGKIICCFVKPVKEFYLHQLYDTSTIL
KLLVFFIKNGTSSQPLIKFSKKYFLSSSSAYRLRESLIKLLREFGLRVSK
NTIVGEEYRIRYLIAMLYSKFGIVIYPLDHLDNQIIYRFLSQSATNLRTS
PWLEEPFSFYNMLLALSWKRHQFAVSIPQTRIFRQLKKLFIYDCLTRSSR
QVIENAFSLMFSQGDLDYLFLIYITTNNSFASLQWTPQHIETCCHIFEKN
DTFRLLLEPILKRLPQLNHSKQDLIKALMYFSKSFLFNLQHFVIEIPSFS
LPTYTGNSNLYKALKNIVNQWLAQLPGKRHLNEKHLQLFCSHIEQILKNK
QPALTVVLISSNFINAKLLTDTIPRYFSDKGIHFYSFYLLRDDIYQIPSL
KPDLVITHSRLIPFVKNDLVKGVTVAEFSFDNPDYSIASIQNLIYQLKDK
KYQDFLNEQLQ

Orf 78 is thought to be a collagen binding protein (Cbp). An example of the nucleotide sequence encoding Cbp (SEQ ID NO:90) and a Cbp amino acid sequence (SEQ ID NO:91) are set forth below.

SEQ ID NO: 90

TTGCAAAAGAGGGATAAAACCAATTATGGAAGCGCTAACAACAAACGACG
ACAAACGACGATCGGATTACTGAAAGTATTTTTGACGTTTGTAGCTCTGA
TAGGAATAGTAGGGTTTTCTATCAGAGCGTTCGGAGCTGAAGAAAAATCT
ACTGAAACTAAAAAAACGTCAGTCATTATTAGAAAATATGCTGAAGGTGA
CTACTCTAAACTTCTAGAGGGAGCAACTTTGCGTTTAACAGGGGAAGATA
TCCCAGATTTTCAAGAAAAAGTCTTCCAAAGTAATGGAACAGGAGAAAAG
ATTGAATTATCAAATGGGACTTATACCTTAACAGAAACATCATCTCCAGA
TGGATATAAAATTACGGAGCCGATTAAGTTTAGAGTAGTGAATAAAAAAG
TATTTATCGTCCAAAAAGATGGTTCTCAAGTGGAAAACCCAAACAAAGAA
CTAGGTTCTCCATATACTATAGAGGCATACAATGATTTTGATGAATTTGG
CTTACTGTCAACACAAAATTATGCGAAATTTTATTATGGAAAAAACTATG
ATGGCAGTTCACAAATTGTTTATTGCTTCAATGCCAACTTGAAATCTCCA
CCTGACTCGGAAGATCATGGTGCTACAATAAATCCTGACTTTACGACTGG
TGATATTAGGTACAGTCATATTGCTGGTTCAGATTTGATAAAATACGCTA
ATACAGCTAGGGATGAAGATCCTCAATTATTTTTAAAACACGTAAAAAAA
GTAATTGAAAATGGGTATCATAAAAAAGGTCAAGCTATTCCATATAACGG
TCTGACTGAGGCACAGTTTCGTGCGGCTACTCAACTGGCAATTTATTATT
TTACAGATAGTGTTGACTTAACTAAGGATAGATTGAAAGACTTCCATGGA
TTTGGAGATATGAATGATCAAACTTTGGGTGTAGCTAAAAAAATTGTAGA
ATACGCTTTGAGTGATGAAGATTCAAAACTAACAAATCTTGATTTCTTCG
TACCTAATAATAGCAAATACCAATCTCTTATTGGGACAGAATACCATCCA
GATGATTTGGTTGACGTGATTCGTATGGAAGATAAAAAGCAAGAAGTTAT
TCCAGTAACTCATAGTTTGACGGTGCAAAAAACAGTAGTCGGTGAGTTGG
GAGATAAGACTAAAGGCTTTCAATTTGAACTTGAGTTGAAAGATAAAACT
GGACAGCCTATTGTTAACACTCTAAAAACTAATAATCAAGATTTAGTAGC
TAAAGATGGGAAATATTCATTTAATCTAAAGCATGGTGACACCATAAGAA
TAGAAGGATTACCGACGGGATATTCTTATACCCTGAAAGAGACTGAAGCT
AAGGATTATATAGTAACTGTTGATAACAAAGTTAGTCAAGAAGCTCAATC
AGCAAGTGAGAATGTCACAGCAGACAAAGAAGTCACTTTTGAAAACCGAA
AAGATCTTGTCCCACCAACTGGTTTGACAACAGATGGGGCTATCTATCTT
TGGTTATTACTACTTGTTCCATTTGGGTTATTGGTTTGGCTATTTGGTCG
TAAAGGGTTAAAAAATGAC

SEQ ID NO: 91

MQKRDKTNYGSANNKRRQTTIGLLKVFLTFVALIGIVGFSIRAFGAEEKS
TETKKTSVIIRKYAEGDYSKLLEGATLRLTGEDIPDFQEKVFQSNGTGEK
IELSNGTYTLTETSSPDGYKITEPIKFRVVNKKVFIVQKDGSQVENPNKE
LGSPYTIEAYNDFDEFGLLSTQNYAKFYYGKNYDGSSQIVYCFNANLKSP
PDSEDHGATINPDFTTGDIRYSHIAGSDLIKYANTARDEDPQLFLKHVKK
VIENGYHKKGQAIPYNGLTEAQFRAATQLAIYYFTDSVDLTKDRLKDFHG
FGDMNDQTLGVAKKIVEYALSDEDSKLTNLDFFVPNNSKYQSLIGTEYHP
DDLVDVIRMEDKKQEVIPVTHSLTVQKTVVGELGDKTKGFQFELELKDKT
GQPIVNTLKTNNQDLVAKDGKYSFNLKHGDTIRIEGLPTGYSYTLKETEA
KDYIVTVDNKVSQEAQSASENVTADKEVTFENRKDLVPPTGLTTDGAIYL
WLLLLVPFGLLVWLFGRKGLKND

Orf 78 contains an amino acid motif indicative of a cell wall anchor: SEQ ID NO:184 VPPTG (shown in italics in SEQ ID NO:91, above). In some recombinant host cell systems, it may be preferable to remove this motif to facilitate secretion of a recombinant Orf 78 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Three E boxes containing conserved glutamic residues have been identified in Orf 78. The E-box motifs are underlined in SEQ ID NO:91, below. The conserved glutamic acid (E) residues, at amino acid residues 112, 395, and 447, are marked in bold. The E box motifs, in particular the conserved glutamic acid residues, are thought to be important for the formation of oligomeric pilus-like structures of Orf 78. Preferred fragments of Orf 78 include at least one conserved glutamic acid residue. Preferably, fragments include at least one E box motif.

SEQ ID NO: 91

MQKRDKTNYGSANNKRRQTTIGLLKVFLTFVALIGIVGFSIRAFGAEEKS
TETKKTSVIIRKYAEGDYSKLLEGATLRLTGEDIPDFQEKVFQSNGTGEK
IELSNGTYTLTETSSPDGYKITEPIKFRVVNKKVFIVQKDGSQVENPNKE
LGSPYTIEAYNDFDEFGLLSTQNYAKFYYGKNYDGSSQIVYCFNANLKSP
PDSEDHGATINPDFTTGDIRYSHIAGSDLIKYANTARDEDPQLFLKHVKK
VIENGYHKKGQAIPYNGLTEAQFRAATQLAIYYFTDSVDLTKDRLKDFHG
FGDMNDQTLGVAKKIVEYALSDEDSKLTNLDFFVPNNSKYQSLIGTEYHP
DDLVDVIRMEDKKQEVIPVTHSLTVQKTVVGELGDKTKGFQFELELKDKT
GQPIVNTLKTNNQDLVAKDGKYSFNLKHGDTIRIEGLPTGYSYTLKETEA
KDYIVTVDNKVSQEAQSASENVTADKEVTFENRKDLVPPTGLTTDGAIYL
WLLLLVPFGLLVWLFGRKGLKND

Orf 79 is thought to be a LepA signal peptidase I. An example of the nucleotide sequence encoding a LepA signal peptidase I (SEQ ID NO:92) and a LepA signal peptidase I amino acid sequence (SEQ ID NO:93) are set forth below.

SEQ ID NO: 92

ATGACTAATTACCTAAATCGTTTAAATGAGAATTCACTATTTAAAGCTTT
CATACGGTTAGTACTTAAGATTTCTATTATTGGGTTTCTAGGTTACATTC
TATTTCAGTATGTTTTTGGTGTTATGATTATTAACACTAATGATATGAGT
CCTGCTTTAAGTGCAGGTGACGGTGTTTTATATTATCGTTTGACTGATCG
CTATCATATTAATGATGTGGTGGTCTATGAGGTTGATAACACTTTGAAAG
TTGGTCGAATTGTCGCTCAAGCTGGCGATGAGGTTAGTTTTACGCAAGAA
GGAGGACTGTTGATTAATGGGCATCCACCAGAAAAAGAGGTCCCTTACCT
GACGTATCCTCACTCAAGTGGCCCAAACTTTCCCTATAAAGTTCCTACGG
GTAAGTATTTCATATTGAATGATTATCGTGAAGAACGTTTGGACAGTCGT
TATTATGGGGCGTTACCCGTCAATCAAATAAAAGGGAAAATCTCAACTCT
ATTAAGAGTGAGAGGAATT

SEQ ID NO: 93

MTNYLNRLNENSLFKAFIRLVLKISIIGFLGYILFQYVFGVMIININDMS
PALSAGDGVLYYRLTDRYHINDVVVYEVDNTLKVGRIVAQAGDEVSFTQE
GGLLINGHPPEKEVPYLTYPHSSGPNFPYKVPTGKYFILNDYREERLDSR
YYGALPVNQIKGKISTLLRVRGI

Orf 80 is thought to be a fimbrial protein. An example of the nucleotide sequence encoding the fimbrial protein (SEQ ID NO:94) and a fimbrial protein amino acid sequence (SEQ ID NO:95) are set forth below.

SEQ ID NO: 94

TTGGAGAGAGAAAAAATGAAAAAAAACAAATTATTACTTGCTACTGCAAT
CTTAGCAACTGCTTTAGGAACAGCTTCTTTAAATCAAAACGTAAAAGCTG
AGACGGCAGGGGTTGTAACAGGAAAATCACTACAAGTTACAAAGACAATG
ACTTATGATGATGAAGAGGTGTTAATGCCCGAAACCGCCTTTACTTTTAC
TATAGAGCCTGATATGACTGCAAGTGGAAAAGAAGGCAGCCTAGATATTA
AAAATGGAATTGTAGAAGGCTTAGACAAACAAGTAACAGTAAAATATAAG
AATACAGATAAAACATCTCAAAAAACTAAAATAGCACAATTTGATTTTTC
TAAGGTTAAATTTCCAGCTATAGGTGTTTACCGCTATATGGTTTCAGAGA
AAAACGATAAAAAAGACGGAATTACGTACGATGATAAAAAGTGGACTGTA
GATGTTTATGTTGGGAATAAGGCCAATAACGAAGAAGGTTTCGAAGTTCT
ATATATTGTATCAAAAGAAGGTACTTCTAGTACTAAAAAACCAATTGAAT
TTACAAACTCTATTAAAACTACTTCCTTAAAAATTGAAAAACAAATAACT
GGCAATGCAGGAGATCGTAAAAAATCATTCAACTTCACATTAACATTACA
ACCAAGTGAATATTATAAAACTGGATCAGTTGTGAAAATCGAACAGGATG
GAAGTAAAAAAGATGTGACGATAGGAACGCCTTACAAATTTACTTTGGGA
CACGGTAAGAGTGTCATGTTATCGAAATTACCAATTGGTATCAATTACTA
TCTTAGTGAAGACGAAGCGAATAAAGACGGCTACACTACAACGGCAACAT
TAAAAGAACAAGGCAAAGAAAAGAGTTCCGATTTCACTTTGAGTACTCAA
AACCAGAAAACAGACGAATCTGCTGACGAAATCGTTGTCACAAATAAGCG
TGACACTCAAGTTCCAACTGGTGTTGTAGGGACCCTTGCTCCATTTGCAG
TTCTTAGCATTGTGGCTATTGGTGGAGTTATCTATATTACAAAACGTAAA
AAAGCT

SEQ ID NO: 95
1MEREKMKKNKLLLATAILATALGTASLNQNVKAETAGVVTGKSLQVTKTM
TYDDEEVLMPETAFTFTIEPDMTASGKEGSLDIKNGIVEGLDKQVTVKYK
NTDKTSQKTKIAQFDFSKVKFPAIGVYRYMVSEKNDKKDGITYDDKKWTV
DVYVGNKANNEEGFEVLYIVSKEGTSSTKKPIEFTNSIKTTSLKIEKQIT
GNAGDRKKSFNFTLTLQPSEYYKTGSVVKIEQDGSKKDVTIGTPYKFTLG
HGKSVMLSKLPIGINYYLSEDEANKDGYTTTATLKEQGKESSDFTLSTQN
QKTDESADEIVVTNKRDTQVPTGVVGTLAPFAVLSIVAIGGVIYITKRKK
A

Orf 82 contains an amino acid motif indicative of a cell wall anchor: SEQ ID NO:140 QVPTG (shown in italics in SEQ ID NO:95, above). In some recombinant host cell systems, it may be preferable to remove this motif to facilitate secretion of a recombinant Orf 82 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

An E box containing a conserved glutamic residue has been identified in Orf 80. The E-box motif is underlined in SEQ ID NO:95, below. The conserved glutamic acid (E), at amino acid residue 270, is marked in bold. The E box motif, in particular the conserved glutamic acid residue, is thought to be important for the formation of oligomeric pilus-like structures of Orf 80. Preferred fragments of Orf 80 include at least one conserved glutamic acid residue. Preferably, fragments include at least one E box motif.

SEQ ID NO: 95

MEREKMKKNKLLLATAILATALGTASLNQNVKAETAGVVTGKSLQVTKTM
TYDDEEVLMPETAFIFTIEPDMTASGKEGSLDIKNGIVEGLDKQVTVKYK
NTDKTSQKTKIAQFDFSKVKFPAIGVYRYMVSEKNDKKDGITYDDKKWTV
DVYVGNKANNEEGFEVLYIVSKEGTSSTKKPIEFTNSIKTTSLKIEKQIT
GNAGDRKKSFNFTLTLQPSEYYKTGSVVKIEQDGSKKDVTIGTPYKFTLG
HGKSVMLSKLPIGINYYLSEDEANKDGYTTTATLKEQGKEKSSDFTLSTQ
NQKTDESADEIVVTNKRDTQVPTGVVGTLAPFAVLSIVAIGGVIYITKRK
KA

Orf 81 is thought to be a SrtC2 type sortase. An example of the nucleotide sequence encoding the SrtC2 sortase (SEQ ID NO:96) and a SrtC2 sortase amino acid sequence (SEQ ID NO:97) are set forth below.

SEQ ID NO: 96

GTGATTAGTCAAAGAATGATGATGACAATTGTACAGGTTATCAATAAAGC
CATTGATACTCTCATTCTTATCTTTTGTTTAGTCGTACTATTTTTAGCTG
GTTTTGGTTTGTGGGATTCTTATCATCTCTATCAACAAGCAGACGCTTCT
AATTTCAAAAAATTTAAAACAGCTCAACAACAGCCTAAATTTGAAGACTT
GTTAGCTTTGAATGAGGATGTCATTGGTTGGTTAAATATCCCAGGGACTC
ATATTGATTATCCTCTAGTTCAGGGAAAAACGAATTTAGAGTATATTAAT
AAAGCAGTTGATGGCAGTGTTGCCATGTCTGGTAGTTTATTTTTAGATAC
ACGGAATCATAATGATTTTACGGACGATTACTCTCTGATTTATGGCCATC
ATATGGCAGGTAATGCCATGTTTGGCGAAATTCCAAAATTTTTAAAAAAG
GATTTTTTCAACAAACATAATAAAGCTATCATTGAAACAAAAGAGAGAAA
AAAACTAACCGTCACTATTTTTGCTTGTCTCAAGACAGATGCCTTTGACC
AGTTAGTTTTTAATCCTAATGCTATTACCAATCAAGACCAACAAAAGCAG
CTCGTTGATTATATCAGTAAAAGATCAAAACAATTTAAACCTGTTAAATT
GAAGCATCATACAAAGTTCGTTGCTTTTTCAACGTGTGAAAATTTTTCTA
CTGACAATCGTGTTATCGTTGTCGGTACTATTCAAGAA

SEQ ID NO: 97

MISQRMMMTIVQVINKAIDTLILIFCLVVLFLAGFGLWDSYHLYQQADAS
NFKKFKTAQQQPKFEDLLALNEDVIGWLNIPGTHIDYPLVQGKTNLEYIN
KAVDGSVAMSGSLFLDTRNHNDFTDDYSLIYGHHMAGNANFGEIPKFLKK
DFFNKHNKAIIETKERKKLTVTIFACLKTDAFDQLVFNPNAITNQDQQKQ
LVDYISKRSKQFKPVKLKHHTKFVAFSTCENFSTDNRVIVVGTIQE

Orf 82 is referred to as a hypothetical protein. It contains a sortase substrate motif LPXAG shown in italics in SEQ ID NO:99. An example of the nucleotide sequence encoding the hypothetical protein (SEQ ID NO:98) and a hypothetical protein amino acid sequence (SEQ ID NO:99) are set forth below.

SEQ ID NO: 98

TTGCTTTTTCAACGTGTGAAAATTTTTCTACTGACAATCGTGTTATCGTT
GTCGGTACTATTCAAGAATAACGAAAGGAGGAGACTTTTGAGAAAATATT
GGAAAATGTTATTTTCTGTCGTAATGATATTAACCATGCTGGCCTTTAAT
CAGACTGTTTTAGCAAAAGACAGCACTGTTCAAACTAGCATTAGTGTCGA
AAATGTCTTAGAGAGAGCAGGCGATAGTACCCCATTTTCGGTTGCATTAG
AATCAATTGATGCGATGAAAACAATAGACGAAATAACAATTGCTGGTTCT
GGAAAAGCAAGCTTTTCCCCTCTGACCTTCACAACAGTTGGGCAATATAC
TTATCGTGTTTATCAGAAGCCTTCACAAAATAAAGATTATCAAGCAGATA
CTACTGTATTTGACGTTCTTGTCTATGTGACCTATGATGAAGATGGGACT
CTAGTCGCAAAAGTTATTTCTCGAAGGGCTGGAGACGAAGAAAAATCAGC
GATTACTTTTAAGCCCAAACGGTTAGTAAAACCAATACCGCCTAGACAAC
CTAACATCCCTAAAACCCCATTACCATTAGCTGGTGAAGTAAAAAGTTTA
TTGGGTATCTTAAGTATCGTATTACTGGGGTTACTAGTTCTTCTTTATGT
TAAAAAACTGAAGAGTAGGCTA

SEQ ID NO: 99

MLFQRVKIFLLTIVLSLSVLFKNNERRRLLRKYWKMLFSVVMILTMLAFN
QTVLAKDSTVQTSISVENVLERAGDSTPFSVALESIDAMKTIDEITIAGS
GKASFSPLTFTTVGQYTYRVYQKPSQNKDYQADTTVFDVLVYVTYDEDGT
LVAKVISRRAGDEEKSAITFKPKRLVKPIPPRQPNIPKTPLPLAGEVKSL
LGILSIVLLGLLVLLYVKKLKSRL

Orf 82 contains an amino acid motif indicative of a cell wall anchor: SEQ ID NO:185 LPLAG (shown in italics in SEQ ID NO:99, above). In some recombinant host cell systems, it may be preferable to remove this motif to facilitate secretion of a recombinant Orf 82 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in Orf 82. The pilin motif sequence is underlined in SEQ ID NO:99, below. Conserved lysine (K) residues are also marked in bold, at amino acid residues 173 and 188. The pilin sequence, in particular the conserved lysine residues, are thought to be important for the formation of oligomeric, pilus-like structures. Preferred fragments of Orf 82 include at least one conserved lysine residue. Preferably, fragments include the pilin sequence.

SEQ ID NO: 99

MLFQRVKIFLLTIVLSLSVLFKNNERRRLLRKYWKMLFSVVMILTMLAFN
QTVLAKDSTVQTSISVENVLERAGDSTPFSVALESIDAMKTIDEITIAGS
GKASFSPLTFTTVGQYTYRVYQKPSQNKDYQADTTVFDVLVYVTYDEDGT
LVAKVISRRAGDEEKSAITFKPKRLVKPIPPRQPNIPKTPLPLAGEVKSL
LGILSIVLLGLLVLLYVKKLKSRL

An E box containing a conserved glutamic residue has been identified in Orf 82. The E-box motif is underlined in SEQ ID NO:99, below. The conserved glutamic acid (E), at amino acid residue 163, is marked in bold. The E box motif, in particular the conserved glutamic acid residue, is thought to be important for the formation of oligomeric pilus-like structures of Orf 82. Preferred fragments of Orf 82 include the conserved glutamic acid residue. Preferably, fragments include the E box motif.

SEQ ID NO: 99

MLFQRVKIFLLTIVLSLSVLFKNNERRRLLRKYWKMLFSVVMILTMLAFN
QTVLAKDSTVQTSISVENVLERAGDSTPFSVALESIDAMKTIDEITIAGS
GKASFSPLTFTTVGQYTYRVYQKPSQNKDYQADTTVFDVLVYVTYDEDGT
LVAKVISRRAGDEEKSAITFKPKRLVKPIPPRQPNIPKTPLPLAGEVKSL
LGTLSIVLLGLLVLLYVKKLKSRL

Orf 83 is thought to be a multiple sugar metabolism regulator protein. An example of a nucleotide sequence encoding the sugar metabolism regulator protein (SEQ ID NO:100) and a sugar metabolism regulator protein amino acid sequence (SEQ ID NO:101) are set forth below.

SEQ ID NO: 100

ATGATACAACTAAGGATGGGGGCAATCTATCAAATGGTTATATTCGATTT
AAAACATGTGCAAACATTACACAGCTTGTCTCAATTACCTATTTCAGTGA
TGTCACAAGATAAGGCACTTATTCAAGTATATGGTAATGACGACTATTTA
TTATGTTACTATCAATTTTTAAAGCATCTAGCTATTCCTCAAGCTGCACA
AGATGTTATTTTTTATGAGGGTTTATTTGAAGAGTCCTTTATGATTTTTC
CTCTTTGTCACTACATTATTGCCATTGGACCTTTCTATCCTTATTCACTT
AATAAAGACTATCAGGAACAATTAGCTAATAATTTTTTAAAACATTCTTC
TCATCGTAGCAAAGAAGAGCTCTTGTCCTATATGGCACTTGTCCCACATT
TTCCAATTAATAATGTGCGGAACCTTTTGATAGCTATTGACGCTTTTTTT
GACACACAATTTGAGACGACTTGCCAACAAACGATTCATCAATTGTTGCA
GCATTCAAAACAGATGACTGCTGATCCTGATATCATTCATCGCCTTAAGC
ATATTAGCAAAGCATCTAGCCAATTACCGCCTGTTTTAGAGCACCTAAAT
CATATTATGGATCTGGTAAAGCTAGGCAATCCACAATTGCTCAAGCAAGA
AATCAATCGCATCCCCTTATCAAGTATCACCTCATCTTCTATTTCTGCTC
TAAGGGCGGAAAAGAACCTCACTGTTATCTATTTAACTAGGTTACTGGAA
TTCAGTTTTGTAGAAAATACTGACGTAGCAAAGCATTATAGCCTTGTCAA
ATACTACATGGCCTTAAATGAAGAAGCGAGTGACTTGCTCAAAGTTTTGA
GAATTCGCTGTGCAGCTATCATCCATTTTTCCGAATCATTAACCAATAAA
AGTATTTCTGATAAACGTCAAATGTACAATAGTGTGCTTCATTATGTCGA
TAGTCACCTGTATTCCAAATTAAAGGTATCTGATATCGCTAAGCGCCTAT
ATGTTTCCGAATCTCACTTACGTTCAGTCTTTAAAAAATACTCAAATGTT
TCCTTACAACATTATATTCTAAGTACAAAAATCAAAGAAGCTCAACTACT
CTTAAAACGAGGAATTCCTGTTGGAGAAGTGGCTAAAAGCTTATATTTTT
ATGACACTACCCATTTTCATAAAATCTTTAAAAAATACACGGGTATTTCT
TCAAAAGACTATCTTGCTAAATACCGAGATAATATT

SEQ ID NO: 101

MIQLRMGAIYQMVIFDLKHVQTLHSLSQLPISVMSQDKALIQVYGNDDYL
LCYYQFLKHLAIPQAAQDVIFYEGLFEESFMIFPLCHYIIAIGPFYPYSL
NKDYQEQLANNFLKHSSHRSKEELLSYMALVPHFPINNVRNLLIAIDAF
FDTQFETTCQQTIHQLLQHSKQMTADPDIIHRLKHISKASSQLPPVLEH
LNHIMDLVKLGNPQLLKQEINRIPLSSITSSSISALRAEKNLTVIYLTR
LLEFSFVENTDVAKHYSLVKYYMALNEEASDLLKVLRIRCAAIIHFSES
LTNKSISDKRQMYNSVLHYVDSHLYSKLKVSDIAKRLYVSESHLRSVFK
KYSNVSLQHYILSTKIKEAQLLLKRGIPVGEVAKSLYFYDTTHFHKIFK
KYTGISSKDYLAKYRDNI

Orf 84 is thought to be a F2-like fibronectin-binding protein. An example of a nucleotide sequence encoding the F2-like fibronectin-binding protein (SEQ ID NO:102) and a F2-like fibronectin-binding protein amino acid sequence (SEQ ID NO:103) are set forth below.

SEQ ID NO: 102

ATGACACAAAAAAATAGCTATAAGTTAAGCTTCCTGTTATCCCTAACAGG
ATTTATTTTAGGTTTATTATTGGTTTTTATAGGATTGTCCGGAGTATCAG
TAGGACATGCGGAAACAAGAAATGGAGCAAACAAACAAGGAGCTTTTGAA
ATCAAGAAAAATAAAAGTCAAGAAGAATATAATTATGAAGTTTATGATAA
CAGAAACATACTTCAGGATGGGGAACATAAACTTGAAATAAAAAGAGTTG
ATGGGACAGGTAAAACTTATCAAGGTTTTTGCTTTCAGTTAACGAAAAAT
TTTCCCACTGCTCAAGGTGTAAGTAAAAAGCTGTATAAAAAATTGAGTAG
TAGTGATGAAGAAACACTAAAGCAATATGCCTCTAAGTATACAAGTAATA
GGAGAGGAGATACTAGTGGTAATCTTAAAAAGCAAATTGCTAAGGTTCTG
ACAGAAGGTTACCCAACTAACAAAAGTGATTGGTTAAATGGATTGACTGA
AAACGAAAAAATAGAAGTAACCCAGGATGCAATTTGGTATTTTACAGAAA
CGACAGTTCCGGCTGATAGAAGTTATACGAATCGCAACGTAAATAGTCAA
AAAATGAAAGAAGTGTATCAAAAGCTAATTGATACAACAGATATAGATAA
ATATGAAGATGTACAATTTGATTTATTTGTGCCACAAGATACAAACTTAC
AGGCAGTAATTAGTGTAGAGCCTGTTATCGAAAGCCTTCCTTGGACATCG
TTGAAGCCAATAGCCCAGAAGGATATCACTGCCAAAAAAATCTGGGTAGA
TGCACCTAAAGAAAAACCAATTATTTATTTTAAGCTATATAGACAGCTGC
CTGGAGAAAAGGAAGTAGCAGTGGATGACGCTGAGCTAAAACAGATAAAT
AGTGAAGGTCAACAAGAAATATCAGTAACTTGGACAAATCAACTTGTTAC
AGATGAAAAAGGAATGGCTTACATTTATTCTGTAAAAGAAGTAGATAAAA
ATGGCGAGTTACTTGAGCCAAAAGATTATATCAAGAAGGAAGATGGACTT
ACAGTTACTAATACTTATGTAAAGCCAACTAGTGGGCACTATGATATAGA
AGTGACATTTGGAAATGGACATATTGATATTACAGAAGATACTACACCAG
ATATTGTTTCAGGTGAAAACCAAATGAAGCAAATAGAGGGAGAAGATAGT
AAGCCTATTGATGAAGTAACGGAAAATAATTTAATTGAATTTGGTAAAAA
CACGATGCCAGGTGAAGAAGATGGCACAAATTCTAATAAGTATGAAGAAG
TCGAAGACTCACGCCCAGTTGATACCTTGTCAGGTTTATCAAGTGAGCAA
GGTCAGTCCGGTGATATGACAATTGAAGAAGATAGTGCTACCCATATTAA
ATTCTCAAAACGTGATATTGACGGCAAAGAGTTAGCTGGTGCAACTATGG
AGTTGCGTGATTCATCTGGTAAAACTATTAGTACATGGATTTCAGATGGA
CAAGTGAAAGATTTCTACCTGATGCCAGGAAAATATACATTTGTCGAAAC
CGCAGCACCAGACGGTTATGAGATAGCAACTGCTATTACCTTTACAGTTA
ATGAGCAAGGTCAGGTTACTGTAAATGGCAAAGCAACTAAAGGTGACGCT
CATATTGTCATGGTTGATGCTTACAAGCCAACTAAGGGTTCAGGTCAGGT
TATTGATATTGAAGAAAAGCTTCCAGACGAGCAGGGCCATTCTGGCTCAA
CTACTGAAATAGAAGATAGCAAGTCTTCAGACGTTATCATTGGTGGTCAG
GGGCAGATTGTCGAGACAACAGAGGATACCCAAACTGGCATGCACGGGGA
TTCTGGTTGTAAAACGGAAGTCGAAGATACTAAACTAGTACAATCCTTCC
ACTTTGATAACAAGGAATCAGAAAGTAACTCTGAGATTCCTAAAAAAGAT
AAGCCAAAGAGTAATACTAGTTTACCAGCAACTGGTGAGAAGCAACATAA
TATGTTCTTTTGGATGGTTACTTCTTGCTCACTTATTAGTAGTGTTTTTG
TAATATCACTAAAAACTAAAAAACGCCTATCATCATGT

SEQ ID NO: 103

MTQKNSYKLSFLLSLTGFILGLLLVFIGLSGVSVGHAETRNGANKQGAFE
IKKNKSQEEYNYEVYDNRNILQDGEHKLEIKRVDGTGKTYQGFCFQLTKN
FPTAQGVSKKLYKKLSSSDEETLKQYASKYTSNRRGDTSGNLKKQIAKVL
TEGYPTNKSDWLNGLTENEKIEVTQDAIWYFTETTVPADRSYTNRNVNSQ
KMKEVYQKLIDTTDIDKYEDVQFDLFVPQDTNLQAVISVEPVIESLPWTS
LKPIAQKDITAKKIWVDAPKEKPIIYFKLYRQLPGEKEVAVDDAELKQIN
SEGQQEISVTWTNQLVTDEKGMAYIYSVKEVDKNGELLEPKDYIKKEDGL
TVTNTYVKPTSGHYDIEVTFGNGHIDITEDTTPDIVSGENQMKQIEGEDS
KPIDEVTENNLIEFGKNTMPGEEDGTNSNKYEEVEDSRPVDTLSGLSSEQ
GQSGDMTIEEDSATHIKFSKRDIDGKELAGATMELRDSSGKTISTWISDG
QVKDFYLMPGKYTFVETAAPDGYEIATAITFTVNEQGQVTVNGKATKGDA
HIVMVDAYKPTKGSGQVIDIEEKLPDEQGHSGSTTEIEDSKSSDVIIGGQ
GQIVETTEDTQTGMHGDSGCKTEVEDTKLVQSFHFDNKESESNSEIPKKD
KPKSNTSLPATGEKQHNMFFWMVTSCSLISSVFVISLKTKKRLSSC

Orf 84 contains an amino acid motif indicative of a cell wall anchor: SEQ ID NO:181 LPATG (shown in italics in SEQ ID NO:103, above). In some recombinant host cell systems, it may be preferable to remove this motif to facilitate secretion of a recombinant Orf 84 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in Orf 84. The pilin motif sequence is underlined in SEQ ID NO:103, below. A conserved lysine (K) residue is also marked in bold, at amino acid residue 270. The pilin sequence, in particular the conserved lysine residue, is thought to be important for the formation of oligomeric, pilus-like structures. Preferred fragments of Orf 84 include the conserved lysine residue. Preferably, fragments include the pilin sequence.

SEQ ID NO: 103

MTQKNSYKLSFLLSLTGFILGLLLVFIGLSGVSVGHAETRNGANKQGAFE
IKKNKSQEEYNYEVYDNRNILQDGEHKLEIKRVDGTGKTYQGFCFQLTKN
FPTAQGVSKKLYKKLSSSDEETLKQYASKYTSNRRGDTSGNLKKQIAKVL
TEGYPTNKSDWLNGLTENEKIEVTQDAIWYFTETTVPADRSYTNRNVNSQ
KMKEVYQKLIDTTDIDKYEDVQFDLFVPQDTNLQAVISVEPVIESLPWTS
LKPIAQKDITAKKIWVDAPKEKPIIYFKLYRQLPGEKEVAVDDAELKQIN
SEGQQEISVTWTNQLVTDEKGMAYIYSVKEVDKNGELLEPKDYIKKEDGL
TVTNTYVKPTSGHYDIEVTFGNGHIDITEDTTPDIVSGENQMKQIEGEDS
KPIDEVTENNLIEFGKNTMPGEEDGTNSNKYEEVEDSRPVDTLSGLSSEQ
GQSGDMTIEEDSATHIKFSKRDIDGKELAGATMELRDSSGKTISTWISDG
QVKDFYLMPGKYTFVETAAPDGYEIATAITFTVNEQGQVTVNGKATKGDA
HIVMVDAYKPTKGSGQVIDIEEKLPDEQGHSGSTTEIEDSKSSDVIIGGQ
GQIVETTEDTQTGMHGDSGCKTEVEDTKLVQSFHFDNKESESNSEIPKKD
KPKSNTSLPATGEKQHNMGGWMVTSCSLISSVFVISLKTKKRLSSC

An E box containing a conserved glutamic residue has been identified in Orf 84. The E-box motif is underlined in SEQ ID NO:103, below. The conserved glutamic acid (E), at amino acid residue 516, is marked in bold. The E box motif, in particular the conserved glutamic acid residue, is thought to be important for the formation of oligomeric pilus-like structures of Orf 84. Preferred fragments of Orf 84 include the conserved glutamic acid residue. Preferably, fragments include the E box motif.

SEQ ID NO: 103

MTQKNSYKLSFLLSLTGFILGLLLVFIGLSGVSVGHAETRNGANKQGAFE
IKKNKSQEEYNYEVYDNRNILQDGEHKLEIKRVDGTGKTYQGFCFQLTKN
FPTAQGVSKKLYKKLSSSDEETLKQYASKYTSNRRGDTSGNLKKQIAKVL
TEGYPTNKSDWLNGLTENEKIEVTQDAIWYFTETTVPADRSYTNRNVNSQ
KMKEVYQKLIDTTDIDKYEDVQFDLFVPQDTNLQAVISVEPVIESLPWTS
LKPIAQKDITAKKIWVDAPKEKPIIYFKLYRQLPGEKEVAVDDAELKQIN
SEGQQEISVTWTNQLVTDEKGMAYIYSVKEVDKNGELLEPKDYIKKEDGL
TVTNTYVKPTSGHYDIEVTFGNGHIDITEDTTPDIVSGENQMKQIEGEDS
KPIDEVTENNLIEFGKNTMPGEEDGTNSNKYEEVEDSRPVDTLSGLSSEQ
GQSGDMTIEEDSATHIKFSKRDIDGKELAGATMELRDSSGKTISTWISDG
QVKDFYLMPGKYTFVETAAPDGYEIATAITFTVNEQGQVTVNGKATKGDA
HIVMVDAYKPTKGSGQVIDIEEKLPDEQGHSGSTTEIEDSKSSDVIIGGQ
GQIVETTEDTQTGMHGDSGCKTEVEDTKLVQSFHFDNKESESNSEIPKKD
KPKSNTSLPATGEKQHNMGGWMVTSCSLISSVFVISLKTKKRLSSC

Examples of GAS AI-3 sequences from M18 strain isolate MGAS8232 are set forth below.

SpyM18_{—}0125 is a negative transcriptional regulator (Nra). An example of SpyM18_{—}0125 is set forth in SEQ ID NO:72.

SEQ ID NO: 72

MPYVKKKKDSFLVETYLEQSIRDKSELVLLLFKSPTIIFSHVAKQTGLTA
VQLKYYCKELDDFFGNNLDITIKKGKIICCFVKPVKEFYLHQLYDTSTIL
KLLVFFIKNGTTSQPLIKFSKKYFLSSSSAYRLRESLIKLLREFGLRVSK
NTIVGEEYRIRYLIAMLYSKFGIVIYPLDHLDNQIIYRFLSQSATNLRTS
PWLEEPFSFYNMLLALS

SpyM18_{—}0126 is thought to be a collagen binding protein (CBP). An example of SpyM18_{—}0126 is set forth in SEQ ID NO:73.

SEQ ID NO: 73

MQKRDKTNYGSANNKRRQTTIGLLKVFLTFVALIGIVGFSIRAFGAEEQS
TETKKTSVIIRKYAEGDYSKLLEGATLKLAQIEGSGFQEQSFESSTSGQK
LQLSDGTYILTETKSPQGYEIAEPITFKVTAGKVFIKGKDGQFVENQNKE
VAEPYSVTAYNDFDDSGFINPKTFTPYGKFYYAKNANGTSQVVYCFNVDL
HSPPDSLDKGETIDPDFNEGKEIKYTHILGADLFSYANNPRASTNDELLS
QVKKVLEKGYRDDSTTYANLTSVEFRAATQLAIYYFTDSVDLDNLADYHG
FGALTTEALNATKEIVAYAEDPANLPNISNLDFYVPNSNKYQSLIGTQYH
PESLVDIIRMEDKQAPIIPITHKLTISKTVTGTIADKKKEFNFEIHLKSS
DGQAISGTYPTNSGELTVTDGKATFTLKDGESLIVEGLPSGYSYEITETG
ASDYEVSVNGKNAPDGKATKASVKEDETITFENRKDLVPPTGLTTDGAIY
LWLLLLVLLGLWVWLIGRKGLKND

SpyM18_{—}0126 contains an amino acid motif indicative of a cell wall anchor: SEQ ID NO:184 VPPTG (shown in italics in SEQ ID NO:73, above). In some recombinant host cell systems, it may be preferable to remove this motif to facilitate secretion of a recombinant SpyM18_{—}0126 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in SpyM18_{—}0126. The pilin motif sequence is underlined in SEQ ID NO:73, below. Conserved lysine (K) residues are also marked in bold, at amino acid residues 172 and 179. The pilin sequence, in particular the conserved lysine residues, are thought to be important for the formation of oligomeric, pilus-like structures. Preferred fragments of SpyM18_{—}0126 include at least one conserved lysine residue. Preferably, fragments include the pilin sequence.

SEQ ID NO: 73

MQKRDKTNYGSANNKRRQTTIGLLKVFLTFVALIGIVGFSIRAFGAEEQS
TETKKTSVIIRKYAEGDYSKLLEGATLKLAQIEGSGFQEQSFESSTSGQK
LQLSDGTYILTETKSPQGYEIAEPITFKVTAGKVFIKGKDGQFVENQNKE
VAEPYSVTAYNDFDDSGFINPKTFTPYGKFYYAKNANGTSQVVYCFNVDL
HSPPDSLDKGETIDPDFNEGKEIKYTHILGADLFSYANNPRASTNDELLS
QVKKVLEKGYRDDSTTYANLTSVEFRAATQLAIYYFTDSVDLDNLADYHG
FGALTTEALNATKEIVAYAEDPANLPNISNLDFYVPNSNKYQSLIGTQYH
PESLVDIIRMEDKQAPIIPITHKLTISKTVTGTIADKKKEFNFEIHLKSS
DGQAISGTYPTNSGELTVTDGKATFTLKDGESLIVEGLPSGYSYEITETG
ASDYEVSVNGKNAPDGKATKASVKEDETITFENRKDLVPPTGLTTDGAIY
LWLLLLVLLGLWVWLIGRKGLKND

Three E boxes containing conserved glutamic residues have been identified in SpyM18_{—}0126. The E-box motifs are underlined in SEQ ID NO:73, below. The conserved glutamic acid (E) residues, at amino acid residues 112, 257, and 415, are marked in bold. The E box motifs, in particular the conserved glutamic acid residues, are thought to be important for the formation of oligomeric pilus-like structures of SpyM18_{—}0126. Preferred fragments of SpyM18_{—}0126 include at least one conserved glutamic acid residue. Preferably, fragments include at least one E box motif.

SEQ ID NO: 73

MQKRDKTNYGSANNKRRQTTIGLLKVFLTFVALIGIVGFSIRAFGAEEQS
TETKKTSVIIRKYAEGDYSKLLEGATLKLAQIEGSGFQEQSFESSTSGQK
LQLSDGTYILTETKSPQGYEIAEPITFKVTAGKVFIKGKDGQFVENQNKE
VAEPYSVTAYNDFDDSGFINPKTFTPYGKFYYAKNANGTSQVVYCFNVDL
HSPPDSLDKGETIDPDFNEGKEIKYTHILGADLFSYANNPRASTNDELLS
QVKKVLEKGYRDDSTTYANLTSVEFRAATQLAIYYFTDSVDLDNLADYHG
FGALTTEALNATKEIVAYAEDPANLPNISNLDFYVPNSNKYQSLIGTQYH
PESLVDIIRMEDKQAPIIPITHKLTISKTVTGTIADKKKEFNFEIHLKSS
DGQAISGTYPTNSGELTVTDGKATFTLKDGESLIVEGLPSGYSYEITETG
ASDYEVSVNGKNAPDGKATKASVKEDETITFENRKDLVPPTGLTTDGAIY
LWLLLLVLLGLWVWLIGRKGLKND

SpyM18_{—}0127 is a LepA protein. An example of SpyM18_{—}0127 is shown in SEQ ID NO:74.

SEQ ID NO: 74

MTNYLNRLNENPLFKAFIRLVLKISIIGFLGYILFQYIFGVMIINTNVMS
PALSAGDGILYYRLTDRYHINDVVVYEVDNTLKVGRIVAQAGDEVSFTQE
GGLLINGHPPEKEVPYLTYPHSSGPNFPYKVPTGTYFILNDYREERLDSR
YYGALPINQIKQKISTLLRVRGI

SpyM18_{—}0128 is thought to be a fimbrial protein. An example of SypM18 0128 is shown in SEQ ID NO:75.

SEQ ID NO: 75

MKKNKLLLATAILATALGTASLNQNVKAETAGVIDGSTLVVKKTFPSYTD
DKVLMPKADYTFKVEADDNAKGKTKDGLDIKPGVIDGLENTKTIHYGNSD
KTTAKEKSVNFDFANVKFPGVGVYRYTVSEVNGNKAGIAYDSQQWTVDVY
VVNREDGGFEAKYIVSTEGGQSDKKPVLFKNFFDTTSLKVTKKVTGNTGE
HQRSFSFTLLLTPNECFEKGQVVNILQGGETKKVVIGEEYSFTLKDKESV
TLSQLPVGIEYKVTEEDVTKDGYKTSATLKDGDVTDGYNLGDSKTTDKST
DEIVVTNKRDTQVPTGVVGTLAPFAVLSIVAIGGVIYITKRKKA

SpyM18_{—}0128 contains an amino acid motif indicative of a cell wall anchor: SEQ ID NO:140 QVPTG (shown in italics in SEQ ID NO:75, above). In some recombinant host cell systems, it may be preferable to remove this motif to facilitate secretion of a recombinant SpyM18_{—}0128 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in SpyM18_{—}0128. The pilin motif sequence is underlined in SEQ ID NO:75, below. A conserved lysine (K) residue is also marked in bold, at amino acid residue 57. The pilin sequence, in particular the conserved lysine residue, is thought to be important for the formation of oligomeric, pilus-like structures. Preferred fragments of SpyM18_{—}0128 include the conserved lysine residue. Preferably, fragments include at least one pilin sequence.

SEQ ID NO: 75

MKKNKLLLATAILATALGTASLNQNVKAETAGVIDGSTLVVKKTFPSYTD
DKVLMPKADYTFKVEADDNAKGKTKDGLDIKPGVIDGLENTKTIHYGNSD
KTTAKEKSVNFDFANVKFPGVGVYRYTVSEVNGNKAGIAYDSQQWTVDVY
VVNREDGGFEAKYIVSTEGGQSDKKPVLFKNFFDTTSLKVTKKVTGNTGE
HQRSFSFTLLLTPNECFEKGQVVNILQGGETKKVVIGEEYSFTLKDKESV
TLSQLPVGIEYKVTEEDVTKDGYKTSATLKDGDVTDGYNLGDSKTTDKST
DEIVVTNKRDTOVPTGVVGTLAPFAVLSIVAIGGVIYITKRKKA

An E box containing a conserved glutamic residue has been identified in SpyM18_{—}0128. The E-box motif is underlined in SEQ ID NO:75, below. The conserved glutamic acid (E), at amino acid residue 266, is marked in bold. The E box motif, in particular the conserved glutamic acid residue, is thought to be important for the formation of oligomeric pilus-like structures of SpyM18_{—}0128. Preferred fragments of SpyM18_{—}0128 include the conserved glutamic acid residue. Preferably, fragments include the E box motif.

SEQ ID NO: 75

MKKNKLLLATAILATALGTASLNQNVKAETAGVIDGSTLVVKKTFPSYTD
DKVLMPKADYTFKVEADDNAKGKTKDGLDIKPGVIDGLENTKTIHYGNSD
KTTAKEKSVNFDFANVKFPGVGVYRYTVSEVNGNKAGIAYDSQQWTVDVY
VVNREDGGFEAKYIVSTEGGQSDKKPVLFKNFFDTTSLKVTKKVTGNTGE
HQRSFSFTLLLTPNECFEKGQVVNILQGGETKKVVIGEEYSFTLKDKESV
TLSQLPVGIEYKVTEEDVTKDGYKTSATLKDGDVTDGYNLGDSKTTDKST
DEIVVTNKRDTQVPTGVVGTLAPFAVLSIVAIGGVIYITKRKKA

SpyM18_{—}0129 is a SrtC2 type sortase. An example of SpyM18_{—}0129 is shown in SEQ ID NO:76

SEQ ID NO: 76

MISQRMMMTIVQVINKAIDTLILIFCLVVLFLAGFGLWDSYHLYQQADAS
NFKKFKTAQQQPKFEDLLALNEDVIGWLNIPGTHMDYPLVQGKTNLEYIN
KAVDGSVAMSGSLFLDTRNHNDFTDDYSLIYGHHMAGNANFGEIPKFLKK
DFFNKHNKAIIETKERKKLTVTIFACLKTDAFDQLVFNPNAITNQDQQRQ
LVDYISKRSKQFKPVKLKHHTKFVAFSTCENFSTDNRVIVVGTIQE

SpyM18_{—}0130 is referred to as a hypothetical protein. An example of SpyM18_{—}0130 is shown in SEQ ID NO:77.

SEQ ID NO: 77

MRKYWKMLFSVVMILTMLAFNQTVLAKDSTVQTSISVENVLERAGDSTSF
SVALESIDAMKTIDEITIAGSGKASFSPLTFTTVGQYTYRVYQKPSQNKD
YQADTTVFDVLVYVTYDEDGTLVAKVISRRAGDEEKSAITFKPKRLVKPI
PPRQPDIPKTPLPLAGEVKSLLGILSIVLLGLLVLLYVKKLKSRL

SpyM18_{—}0130 contains an amino acid motif indicative of a cell wall anchor: SEQ ID NO:185 LPLAG (shown in italics in SEQ ID NO:77, above). In some recombinant host cell systems, it may be preferable to remove this motif to facilitate secretion of a recombinant SpyM18_{—}0130 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in SpyM18_{—}0130. The pilin motif sequence is underlined in SEQ ID NO:77, below. Conserved lysine (K) residues are also marked in bold, at amino acid residues 144, 159, and 169. The pilin sequence, in particular the conserved lysine residues, are thought to be important for the formation of oligomeric, pilus-like structures. Preferred fragments of SpyM18_{—}0130 include at least one conserved lysine residue. Preferably, fragments include the pilin sequence.

SEQ ID NO: 77

MRKYWKMLFSVVMILTMLAFNQTVLAKDSTVQTSISVENVLERAGDSTSF
SVALESIDAMKTIDEITIAGSGKASFSPLTFTTVGQYTYRVYQKPSQNKD
YQADTTVFDVLVYVTYDEDGTLVAKVISRRAGDEEKSAITFKPKRLVKPI
PPRQPDIPKTPLPLAGEVKSLLGILSIVLLGLLVLLYVKKLKSRL

An E box containing a conserved glutamic residue has been identified in SpyM18_{—}0130. The E-box motif is underlined in SEQ ID NO:77, below. The conserved glutamic acid (E), at amino acid residue 134, is marked in bold. The E box motif, in particular the conserved glutamic acid residue, is thought to be important for the formation of oligomeric pilus-like structures of SpyM18_{—}0130. Preferred fragments of SpyM18_{—}0130 include the conserved glutamic acid residue. Preferably, fragments include the E box motif.

SEQ ID NO: 77

MRKYWKMLFSVVMILTMLAFNQTVLAKDSTVQTSISVENVLEPAGDSTSF
SVALESIDAMKTIDEITIAGSGKASFSPLTFTTVGQYTYRVYQKPSQNKD
YQADTTVFDVLVYVTYDEDGTLVAKVISRRAGDEEKSAITFKPKRLVKPI
PPRQPDIPKTPLPLAGEVKSLLGILSIVLLGLLVLLYVKKLKSRL

SpyM18_{—}0131 is referred to as a putative multiple sugar metabolism regulator. An example of SpyM18_{—}0131 is set forth in SEQ ID NO:78.

SEQ ID NO: 78

MAIFDLKHVQTLHSLSQLPISVMSQDKALIQVYGNDDYLLCYYQFLKHLA
IPQAAQDVIFYEGLFEESFMIFFLCHYIIAIGPFYPYSLNKDYQEQLANN
CLKHSSHRSKEELLSYMALVPHFPINNVRNLLIAIDAFFDTQFETTCQQT
IHQLLQHSKQMTADPDIIHRLKHISKASSQLPPVLEHLNHIMDLVKLGNP
QLLKQEINRIPLSSITSSSISALRAEKNLTVIYLTRLLEFSFVENTDVAK
HYSLVKYYMALNEEASDLLKVLRIRCAAIIHFSESLTNKSISDKRQMYNS
VLHYVDSHLYSKLKVSDIAKRLYVSESHLRSVFKKYSNVSLQHYILSTKI
KEAQLLLKRGIPVGEVAKSLYFYDTTHFHKIFKKYTGISSKDYLAKYRDN
I

SpyM18_{—}0132 is a F2 like fibronectin-binding protein. An example of SpyM18_{—}0132 is set forth in SEQ ID NO:79.

SEQ ID NO: 79

MTQKNSYKLSFLLSLTGFILGLLLVFIGLSGVSVGHAETRNGANKQGAFE
IKKNKSQEEYNYEVYDNRNILQDGEHKLEIKRVDGTGKTYQGFCFQLTKN
FPTAQGVSKKLYKKLSSSDEETLKQYASKYTSNRRGDTSGNLKKQIAKVL
TEGYPTNKSDWLNGLTENEKIEVTQDAIWYFTETTVPADRSYTNRNVNSQ
KMKEVYQKLIDTTDIDKYEDVQFDLFVPQDTNLQAVISVEPVIESLPWTS
LKPIAQKDITAKKIWVDAPKEKPIIYFKLYRQLPGEKEVAVDDAELKQIN
SEGQQEISVTWTNQLVTDEKGMAYIYSVKEVDKNGELLEPKDYIKKEDGL
TVTNTYVKPTSGHYDIEVTFGNGHIDITEDTTPDIVSGENQMKQIEGEDS
KPIDEVTENNLIEFGKNTMPGEEDGTNSNKYEEVEDSRPVDTLSGLSSEQ
GQSGDMTIEEDSATHIKFSKRDIDGKELAGATMELRDSSGKTISTWISDG
QVKDFYLMPGKYTFVETAAPDGYEIATAITFTVNEQGQVTVNGKATKGDA
HIVMVDAYKPTKGSGQVIDIEEKLPDEQGHSGSTTEIEDSKSSDVIIGGQ
GQIVETTEDTQTGMHGDSGCKTEVEDTKLVQSFHFDNKESESNSEIPKKD
KPKSNTSLPATGEKQHNMFFWMVTSCSLISSVFVISLKTKKRLSSC

SpyM18_{—}0132 contains an amino acid motif indicative of a cell wall anchor: SEQ ID NO:180 LPATG (shown in italics in SEQ ID NO:79, above). In some recombinant host cell systems, it may be preferable to remove this motif to facilitate secretion of a recombinant SpyM18_{—}0132 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in SpyM18_{—}0132. The pilin motif sequence is underlined in SEQ ID NO:79, below. A conserved lysine (K) residue is also marked in bold, at amino acid residue 270. The pilin sequence, in particular the conserved lysine residue, is thought to be important for the formation of oligomeric, pilus-like structures. Preferred fragments of SpyM18_{—}0132 include the conserved lysine residue. Preferably, fragments include the pilin sequence.

SEQ ID NO: 79

MTQKNSYKLSFLLSLTGFILGLLLVFIGLSGVSVGHAETRNGANKQGAFE
IKKNKSQEEYNYEVYDNRNILQDGEHKLEIKRVDGTGKTYQGFCFQLTKN
FPTAQGVSKKLYKKLSSSDEETLKQYASKYTSNRRGDTSGNLKKQIAKVL
TEGYPTNKSDWLNGLTENEKIEVTQDAIWYFTETTVPADRSYTNRNVNSQ
KMKEVYQKLIDTTDIDKYEDVQFDLFVPQDTNLQAVISVEPVIESLPWTS
LKPIAQKDITAKKIWVDAPKEKPIIYFKLYRQLPGEKEVAVDDAELKQIN
SEGQQEISVTWTNQLVTDEKGMAYIYSVKEVDKNGELLEPKDYIKKEDGL
TVTNTYVKPTSGHYDIEVTFGNGHIDITEDTTPDIVSGENQMKQIEGEDS
KPIDEVTENNLIEFGKNTMPGEEDGTNSNKYEEVEDSRPVDTLSGLSSEQ
GQSGDMTIEEDSATHIKFSKRDIDGKELAGATMELRDSSGKTISTWISDG
QVKDFYLMPGKYTFVETAAPDGYEIATAITFTVNEQGQVTVNGKATKGDA
HIVMVDAYKPTKGSGQVIDIEEKLPDEQGHSGSTTEIEDSKSSDVIIGGQ
GQIVETTEDTQTGMHGDSGCKTEVEDTKLVQSFHFDNKESESNSEIPKKD
KPKSNTSLPATGEKQHNMFFWMVTSCLISSVFVISLKTKKRLSSC

An E box containing a conserved glutamic residue has been identified in SpyM18_{—}0132. The E-box motif is underlined in SEQ ID NO:79, below. The conserved glutamic acid (E), at amino acid residue 516, is marked in bold. The E box motif, in particular the conserved glutamic acid residue, is thought to be important for the formation of oligomeric pilus-like structures of SpyM18_{—}0132. Preferred fragments of SpyM18_{—}0132 include the conserved glutamic acid residue. Preferably, fragments include the E box motif.

SEQ ID NO: 79

MTQKNSYKLSFLLSLTGFILGLLLVFIGLSGVSVGHAETRNGANKQGAFE
IKKNKSQEEYNYEVYDNRNILQDGEHKLEIKRVDGTGKTYQGFCFQLTKN
FPTAQGVSKKLYKKLSSSDEETLKQYASKYTSNRRGDTSGNLKKQIAKVL
TEGYPTNKSDWLNGLTENEKIEVTQDAIWYFTETTVPADRSYTNRNVNSQ
KMKEVYQKLIDTTDIDKYEDVQFDLFVPQDTNLQAVISVEPVIESLPWTS
LKPIAQKDITAKKIWVDAPKEKPIIYFKLYRQLPGEKEVAVDDAELKQIN
SEGQQEISVTWTNQLVTDEKGMAYIYSVKEVDKNGELLEPKDYIKKEDGL
TVTNTYVKPTSGHYDIEVTFGNGHIDITEDTTPDIVSGENQMKQIEGEDS
KPIDEVTENNLIEFGKNTMPGEEDGTNSNKYEEVEDSRPVDTLSGLSSEQ
GQSGDMTIEEDSATHIKFSKRDIDGKELAGATMELRDSSGKTISTWISDG
QVKDFYLMPGKYTFVETAAPDGYEIATAITFTVNEQGQVTVNGKATKGDA
HIVMVDAYKPTKGSGQVIDIEEKLPDEQGHSGSTTEIEDSKSSDVIIGGQ
GQIVETTEDTQTGMHGDSGCKTEVEDTKLVQSFHFDNKESESNSEIPKKD
KPKSNTSLPATGEKQHNMFFWMVTSCSLISSVFVISLKTKKRLSSC

Examples of GAS AI-3 sequences from M49 strain isolate 591 are set forth below.

SpyoM01000156 is a negative transcriptional regulator (Nra). An example of SpyoM01000156 is set forth in SEQ ID NO:243.

SEQ ID NO: 243

MPYVKKKKDSFLVETYLEQSIRDKSELVLLLFKSPTIIFSHVAKQTGLTA
VQLKYYCKELDDFFGNNLDITIKKGKIICCFVKPVKEFYLHQLYDTSTIL
KLLVFFIKNGTSSQPLIKFSKKYFLSSSSAYRLRESLIKLLREFGLRVSK
NTIVGEEYRIRYLIAMLYSKFGIVIYPLDHLDNQIIYRFLSQSATNLRTS
PWLEEPFSFYNMLLALSWKRHQFAVSIPQTRIFRQLKKLFIYDCLTRSSR
QVIENAFSLTFSQGDLDYLFLIYITTNNSFASLQWTPQHIETCCHIFEKN
DTFRLLLEPILKRLPQLNHSKQDLIKALMYFSKSFLFNLQHFVIEIPSFS
LPTYTGNSNLYKALKNIVNQWLAQLPGKRHLNEKHLQLFCSHIEQILKNK
QPALTVVLISSNFINAKLLTDTIPRYFSDKGIHFYSFYLLRDDIYQIPSL
KPDLVITHSRLIPFVKNDLVKGVTVAEFSFDNPDYSIASIQNLIYQLKDK
KYQDFLNEQLQ

SpyoM01000155 is thought to be a collagen binding protein (CPA). An example of SpyoM01000155 is set forth in SEQ ID NO:244.

SEQ ID NO: 244

MQKRDKTNYGSANNKRRQTTIGLLKVFLTFVALIGIVGFSIRAFGAEEQS
VPNRQSSIQDYPWYGYDSYPKGYPDYSPLKTYHNLKVNLEGSKDYQAYCF
NLTKHFPSKSDSVRSQWYKKLEGTNENFIKLADKPRIEDGQLQQNILRIL
YNGYPNNRNGIMKGIDPLNAILVTQNAIWYYTDSAQINPDESFKTEARSN
GINDQQLGLMRKALKELIDPNLGSKYSNKTPSGYRLNVFESHDKTFQNLL
SAEYVPDTPPKPGEEPPAKTEKTSVIIRKYAEGDYSKLLEGATLKLSQIE
GSGFQEKDFQSNSLGETVELPNGTYTLTETSSPDGYKIAEPIKFRVENKK
VFIVQKDGSQVENPNKEVAEPYSVEAYNDFMDEEVLSGFTPYGKFYYAKN
KDKSSQVVYCFNADLHSPPDSYDSGETINPDTSTMKEVKYTHTAGSDLFK
YALRPRDTNPEDFLKHIKKVIEKGYKKKGDSYNGLTETQFRAATQLAIYY
FTDSADLKTLKTYNNGKGYHGFESMDEKTLAVTKELITYAQNGSAPQLTN
LDFFVPNNSKYQSLIGTEYHPDDLVDVIRMEDKKQEVIPVTHSLTVKKTV
VGELGDKTKGFQFELELKDKTGQPIVNTLKTNNQDLVAKDGKYSFNLKHG
DTIRIEGLPTGYSYTLKETEAKDYIVTVDNKVSQEAQSVGKDITEDKKVT
FENRKDLVPPTGLTTDGAIYLWLLLLVPLGLLVWLFGRKGLKND

SpyoM01000155 contains an amino acid motif indicative of a cell wall anchor: SEQ ID NO:184 VPPTG (shown in italics in SEQ ID NO:244, above). In some recombinant host cell systems, it may be preferable to remove this motif to facilitate secretion of a recombinant SpyoM01000155 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Two pilin motifs, discussed above, containing conserved lysine (K) residues have also been identified in SpyoM01000155. The pilin motif sequence is underlined in SEQ ID NO:244, below. Conserved lysine (K) residues are also marked in bold, at amino acid residues 71 and 261. The pilin sequences, in particular the conserved lysine residues, are thought to be important for the formation of oligomeric, pilus-like structures. Preferred fragments of SpyoM01000155 include at least one conserved lysine residue. Preferably, fragments include at least one pilin sequence.

SEQ ID NO: 244

MQKRDKTNYGSANNKRRQTTIGLLKVFLTFVALIGIVGFSIRAFGAEEQS
VPNRQSSIQDYPWYGYDSYPKGYPDYSPLKTYHNLKVNLEGSKDYQAYCF
NLTKHFPSKSDSVRSQWYKKLEGTNENFIKLADKPRIEDGQLQQNILRIL
YNGYPNNRNGIMKGIDPLNAILVTQNAIWYYTDSAQINPDESFKTEARSN
GINDQQLGLMRKALKELIDPNLGSKYSNKTPSGYRLNVFESHDKTFQNLL
SAEYVPDTPPKPGEEPPAKTEKTSVIIRKYAEGDYSKLLEGATLKLSQIE
GSGFQEKDFQSNSLGETVELPNGTYTLTETSSPDGYKIAEPIKFRVENKK
VFIVQKDGSQVENPNKEVAEPYSVEAYNDFMDEEVLSGFTPYGKFYYAKN
KDKSSQVVYCFNADLHSPPDSYDSGETINPDTSTMKEVKYTHTAGSDLFK
YALRPRDTNPEDFLKHIKKVIEKGYKKKGDSYNGLTETQFRAATQLAIYY
FTDSADLKTLKTYNNGKGYHGFESMDEKTLAVTKELITYAQNGSAPQLTN
LDFFVPNNSKYQSLIGTEYHPDDLVDVIRMEDKKQEVIPVTHSLTVKKTV
VGELGDKTKGFQFELELKDKTGQPIVNTLKTNNQDLVAKDGKYSFNLKHG
DTIRIEGLPTGYSYTLKETEAKDYIVTVDNKVSQEAQSVGKDITEDKKVT
FENRKDLVPPTGLTTDGAIYLWLLLLVPLGLLVWLFGRKGLKND

Two E boxes containing conserved glutamic residues have been identified in SpyoM01000155. The E-box motifs are underlined in SEQ ID NO:244, below. The conserved glutamic acid (E) residues, at amino acid residues 329 and 668, are marked in bold. The E box motifs, in particular the conserved glutamic acid residues, are thought to be important for the formation of oligomeric pilus-like structures of SpyoM01000155. Preferred fragments of SpyoM01000155 include at least one conserved glutamic acid residue. Preferably, fragments include at least one E box motif.

SEQ ID NO: 244

MQKRDKTNYGSANNKRRQTTIGLLKVFLTFVALIGIVGFSIRAFGAEEQS
VPNRQSSIQDYPWYGYDSYPKGYPDYSPLKTYHNLKVNLEGSKDYQAYCF
NLYKHFPSKSDSVRSQWYKKLEGTNENFIKLADKPRIEDGQLQQNILRIL
YNGYPNNRNGIMKGIDPLNAILVTQNAIWYYTDSAQINPDESFKTEARSN
GINDQQLGLMRKALKELIDPNLGSKYSNKTPSGYRLNVFESHDKTFQNLL
SAEYVPDTPPKPGEEPPAKTEKTSVIIRKYAEGDYSKLLEGATLKLSQIE
GSGFQEKDFQSNSLGETVELPNGTYTLTETSSPDGYKIAEPIKFRVENKK
VFIVQKDGSQVENPNKEVAEPYSVEAYNDFMDEEVLSGFTPYGKFYYAKN
KDKSSQVVYCFNADLHSPPDSYDSGETINPDTSTMKEVKYTHTAGSDLFK
YALFPRDTNPEDFLKHIKKVIEKGYKKKGDSYNGLTETQFRAATQLAIYY
FTDSADLKTLKTYNNGKGYHGFESMDEKTLAVTKELITYAQNGSAPQLTN
LDFFVPNNSKYQSLIGTEYHPDDLVDVIRMEDKKQEVIPVTHSLTVKKTV
VGELGDKTKGFQFELELKDKTGQPIVNTLKTNNQDLVAKDGKYSFNLKHG
DTIRIEGLPTGYSYTLKETEAKDYIVTVDNKVSQEAQSVGKDITEDKKVT
FENRKDLVPPTGLTTDGAIYLWLLLLVPLGLLVWLFGRKGLKND

SpyoM01000154 is a LepA protein. An example of SpyoM01000154 is shown in SEQ ID NO:245.

SEQ ID NO: 245

MTNYLNRLNENSLFKAFIRLVLKISIIGFLGYILFQYVFGVMIINTNDMS
PALSAGDGVLYYRLADRSHINDVVVYEVDNTLKVGRIAAQAGDEVNGTQE
GGLLINGHPPEKEVPYLTYPHSSGPNFPYKVPTGTYFILNDYREERLDSR
YYGALPINQIKGKISTLLRVRGI

SpyoM01000153 is thought to be a fimbrial protein. An example of SpyoM01000153 is shown in SEQ ID NO:246.

SEQ ID NO: 246

NKKNKLLLATAILATALGMASMSQNIKAETAGVIDGSTLVVKKTFPSYTD
DNVLMPKADYSFKVEADDNAKGKTKDGLDIKPGVIDGLENTKTIRYSNSD
KITAKEKSVNFEFANVKFPGVGVYRYTVAEVNGNKAGITYDSQQWTVDVY
VVNKEGGGFEVKYIVSTEVGQSEKKPVLFKNSFDTTSLKIEKQVTGNTGE
HQRLFSFTLLLTPNECFEKGQVVNILQGGETKKVVIGEEYSFTLKDKESV
TLSQLPVGIEYKLTEEDVTKDGYKTSATLKDGEQSSTYELGKDHKTDKSA
DEIVVTNKRDTQVPTGVVGTLAPFAVLSIVAIGGVIYITKRKKA

SpyoM01000153 contains an amino acid motif indicative of a cell wall anchor: SEQ ID NO:140 QVPTG (shown in italics in SEQ ID NO:246, above). In some recombinant host cell systems, it may be preferable to remove this motif to facilitate secretion of a recombinant SpyoM01000153 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in SpyoM01000153. The pilin motif sequence is underlined in SEQ ID NO:246, below. A conserved lysine (K) residue is also marked in bold, at amino acid residue 57. The pilin sequence, in particular the conserved lysine residue, is thought to be important for the formation of oligomeric, pilus-like structures. Preferred fragments of SpyoM01000153 include the conserved lysine residue. Preferably, fragments include the pilin sequence.

SEQ ID NO: 246

MKKNKLLLATAILATALGMASMSQNIKAETAGVIDGSTLVVKKTFPSYTD
DNVLMPKADYSFKVEADDNAKGKTKDGLDIKPGVIDGLENTKTIRYSNSD
KITAKEKSVNFEFANVKFPGVGVYRYTVAEVNGNKAGITYDSQQWTVDVY
VVNKEGGGFEVKYIVSTEVGQSEKKPVLFKNSFDTTSLKIEKQVTGNTGE
HQRLFSFTLLLTPNECFEKGQVVNILQGGETKKVVIGEEYSFTLKDKESV
TLSQLPVGIEYKLTEEDVTKDGYKTSATLKDGEQSSTYELGKDHKTDKSA
DEIVVTNKRDTQVPTGVVGTLAPFAVLSIVAIGGVIYITKRKKA

An E box containing a conserved glutamic residue has been identified in SpyoM01000153. The E-box motif is underlined in SEQ ID NO:246, below. The conserved glutamic acid (E), at amino acid residue 265, is marked in bold. The E box motif, in particular the conserved glutamic acid residue, is thought to be important for the formation of oligomeric pilus-like structures of SpyoM01000153. Preferred fragments of SpyoM01000153 include the conserved glutamic acid residue. Preferably, fragments include the E box motif.

SEQ ID NO: 246

MKKNKLLLATAILATALGMASMSQNIKAETAGVIDGSTLVVKKTFPSYTD
DNVLMPKADYSFKVEADDNAKGKTKDGLDIKPGVIDGLENTKTIRYSNSD
KITAKEKSVNFEFANVKFPGVGVYRYTVAEVNGNKAGITYDSQQWTVDVY
VVNKEGGGFEVKYIVSTEVGQSEKKPVLFKNSFDTTSLKIEKQVTGNTGE
HQRLFSFTLLLTPNECFEKGQVVNILQGGETKKVVIGEEYSFTLKDKESV
TLSQLPVGIEYKLTEEDVTKDGYKTSATLKDGEQSSTYELGKDHKTDKSA
DEIVVTNKRDTQVPTGVVGTLAPFAVLSIVAIGGVIYITKRKKA

SpyoM01000152 is a SrtC2 type sortase. An example of SpyoM01000152 is shown in SEQ ID NO:247

SEQ ID NO: 247

MMMTIVQVINKAIDTLILIFCLVVLFLAGFGLWDSYHLYQQADASNFKKF
KTAQQQPKFEDLLALNEDVIGWLNIPGTHIDYPLVQGKTNLEYINKAVDG
SVAMSGSLFLDTRNHNDFTDDYSLIYGHHMAGNAMFGEIPKFLKKNFFNK
HNKAIIETKERKKLTVTIFACLKTDAFDQLVFNPNAITNQDQQRQLVDYI
SKRSKQFKPVKLKHHTKFVAFSTCENFSTDNRVIVVGTIQE

SpyoM01000151 is referred to as a hypothetical protein. An example of SpyoM01000151 is shown in SEQ ID NO:248.

SEQ ID NO: 248

MLFSVVMMLTMLAFNQTVLAKDSTVQTSISVENVLERAGDSTPFSAILES
IDAMKTIEEITIAGSGKASFSPLTFTTVGQYTYRVYQKPSQNKDYQADTT
VFDVLVYVTYDEDGTLVAKVISRRAGDEEKSAITFKPKRLVKPIPPRQPD
IPKTPLPLAGEVKSLLGILSIVLLGLLVLLYVKKLKSRL

SpyoM01000151 contains an amino acid motif indicative of a cell wall anchor: SEQ ID NO:185 LPLAG (shown in italics in SEQ ID NO:248, above). In some recombinant host cell systems, it may be preferable to remove this motif to facilitate secretion of a recombinant SpyoM01000151 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in SpyoM01000151. The pilin motif sequence is underlined in SEQ ID NO:248, below. Conserved lysine (K) residues are also marked in bold, at amino acid residue 138. The pilin sequence, in particular the conserved lysine residue, is thought to be important for the formation of oligomeric, pilus-like structures. Preferred fragments of SpyoM01000151 include the conserved lysine residue. Preferably, fragments include the pilin sequence.

SEQ ID NO: 248

MLFSVVMMLTMLAFNQTVLAKDSTVQTSISVENVLERAGDSTPFSIALES
IDAMKTIEEITIAGSGKASFSPLTFTTVGQYTYRVYQKPSQNKDYQADTT
VFDVLVYVTYDEDGTLVAKVISRRAGDEEKSAITFKPKRLVKPIPPRQPD
IPKTPLPLAGEVKSLLGILSIVLLGLLVLLYVKKLKSRL

Two E boxes containing conserved glutamic residues have been identified in SpyoM01000151. The E-box motifs are underlined in SEQ ID NO:248, below. The conserved glutamic acid (E) residues, at amino acid residues 58 and 128, are marked in bold. The E box motifs, in particular the conserved glutamic acid residues, are thought to be important for the formation of oligomeric pilus-like structures of SpyoM01000151. Preferred fragments of SpyoM01000151 include at least one conserved glutamic acid residue. Preferably, fragments include at least one E box motif.

SEQ ID NO: 248

MLFSVVMMLTMLAFNQTVLAKDSTVQTSISVENVLERAGDSTPFSIALES
IDAMKTIEEITIAGSGKASFSPLTFTTVGQYTYRVYQKPSQNKDYQADTT
VFDVLVYVTYDEDGTLVAKVISRRAGDEEKSAITFKPKRLVKPIPPRQPD
IPKTPLPLAGEVKSLLGILSIVLLGLLVLLYVKKLKSRL

SpyoM01000150 is referred to as a putative MsmRL. An example of SpyoM01000150 is set forth in SEQ ID NO:249.

SEQ ID NO: 249

MVIFDLKHVQTLHSLSQLPISVMSQDKALIQVYGNDDYLLCYYQFLKHLA
IPQAAQDVIFYEGLFEESFMIFPLCHYIIAIGPFYPYSLNKDYQEQLANN
FLKHSSHRSKEELLSYMALVPHFPINNVRNLLIAIDAFFDTQFETTCQQT
IHQLLQHSKQMTADPDIIHRLKHISKASSQLPPVLEHLNHIMDLVKLGNP
QLLKQEINRIPLSSITSSSISALRAEKNLTVIYLTRLLEFSFVENTDVAK
HYSLVKYYMALNEEASDLLKVLRIRCAAIIHFSESLTNKSISDKRQMYNS
VHLYVDSHLYSKLKVSDIAKRLYVSESHLRSVFKKYSNVSLQHYILSTKI
KEAQLLLKRGIPVGEVAKSLYFYDTTHFHKIFKKYTGISSKDYLAKYRDN
I

SpyoM01000149 is a F2 like fibronectin-binding protein. An example of SpyoM01000149 is set forth in SEQ ID NO:250.

SEQ ID NO: 250

MTQKNSYKLSFLLSLTGFILGLLLVFIGLSGVSVGHAETRNGANKQGYFE
IKKVDQNNKPLSGATFSLTPKDGKGKPVQTFTSSEEGIIDAQNLQPGTYT
LKEETAPDGYDKTSRTWTVTVYENGYTKLVENPYNGEIISKAGSKDVSSS
LQLENPKMSVVSKYGEQEKTSNSADFYRNHAAYFKMSFELKQKDKSETIN
PGDTFVLQLDRRLNPKGISQDIPKIIYDSENSPLAIGKYDAKTHQLTYTF
TNYIAGLDKVQLSAELSLFLENKEVLENTNISDFKSTIGGQEITYKGTVN
VLYGNESTKESNYITNGLSNVGGSIESYNTETGEFVWYVYVNPNRTNIPY
AVLNLWGFAKRTAQGENDNSSVSSAQLTGYDIYEVPHNYRLPTSYGVDIS
RLNLRKDLEAKLPQGSTQGANKRLRIDFGENLQGKAFVVKVTGKADQSGK
ELIVQSHLSSFNNWGSYKTLRPNSHVSFTNEIALSPSKGSGSGTSEFTKP
AITVANLKRVAQLRFKKVSTDNVPLPEAAFELRSSNGNSQKLEASSNTQG
EIHFKDLTSGTYDLYETKAPKGYQQVTEKLATVTVDTTKPAEQMVKWEKP
HSFVKVEANKEVTIVNHKETLTFSGKKIWENDRPDQRPAKIQVQLLQNGQ
KMPNQIQEVTKDNDWSYHFKDLPKYDAKNQEYKYSVEEVKVPDGYKVSYL
GNDIFNTRETEFVFEQNNFNLEFGNAEIKGQSGSKIIDEDTLTSFKGKKI
WKNDTAENRPQAIQVQLYADGVAVEGQTKFISGSGNEWSFEFKNLKKYNG
TGNDIIYSVKEVTVPTGYDVTYSANDIINTKREVITQQGPNLEIEETLPL
ESGASGGTTTVEDSRSVDTLSGLSSEQGQSGDMTIEEDSATHIKFSKRDI
DGKELAGATMELRDSSGKTISTWISDGQVKDFYLMPGKYTFVETAAPDGY
EIATAITFTVNEQGQVTVNGKATKGDAHIVMVDAYKPTKGSGQVIDIEEK
LPDEQGHSGSTTEIEDSKPSDVIIGGQGEVVDTTEDTQSGMTGHSGSTTE
IEDSKSSDVIIGGQGQVVETTEDTQTGMHGDSGCKTEVEDTKLVQFFHFD
NKEPESNSEIPKKDKPKSNTSLPATGEKQHNKFFWMVTSCSLISSVKVIS
LKSKKRLLSC

SpyoM01000149 contains an amino acid motif indicative of a cell wall anchor: SEQ ID NO:180 LPATG (shown in italics in SEQ ID NO:250, above). In some recombinant host cell systems, it may be preferable to remove this motif to facilitate secretion of a recombinant SpyoM01000149 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Two pilin motifs, discussed above, containing conserved lysine (K) residues have also been identified in SpyoM01000149. The pilin motif sequences are underlined in SEQ ID NO:250, below. Conserved lysine (K) residues are also marked in bold, at amino acid residues 157 and 163, and 216 and 224. The pilin sequences, in particular the conserved lysine residues, are thought to be important for the formation of oligomeric, pilus-like structures. Preferred fragments of SpyoM01000149 include at least one conserved lysine residue. Preferably, fragments include at least one pilin sequence.

SEQ ID NO: 250

MTQKNSYKLSFLLSLTGFILGLLLVFIGLSGVSVGHAETRNGANKQGYFE
IKKVDQNNKPLSGATFSLTPKDGKGKPVQTFTSSEEGIIDAQNLQPGTYT
LKEETAPDGYDKTSRTWTVTVYENGYTKLVENPYNGEIISKAGSKDVSSS
LQLENPKMSVVSKYGEQEKTSNSADFYRNHAAYFKMSFELKQKDKSETIN
PGDTFVLQLDRRLNPKGISQDIPKIIYDSENSPLAIGKYDAKTHQLTYTF
TNYIAGLDKVQLSAELSLFLENKEVLENTNISDFKSTIGGQEITYKGTVN
VLYGNESTKESNYITNGLSNVGGSIESYNTETGEFVWYVYVNPNRTNIPY
AVLNLWGFAKRTAQGENDNSSVSSAQLTGYDIYEVPHNYRLPTSYGVDIS
RLNLRKDLEAKLPQGSTQGANKRLRIDFGENLQGKAFVVKVTGKADQSGK
ELIVQSHLSSFNNWGSYKTLRPNSHVSFTNEIALSPSKGSGSGTSEFTKP
AITVANLKRVAQLRFKKVSTDNVPLPEAAFELRSSNGNSQKLEASSNTQG
EIHFKDLTSGTYDLYETKAPKGYQQVTEKLATVTVDTTKPAEQMVKWEKP
HSFVKVEANKEVTIVNHKETLTFSGKKIWENDRPDQRPAKIQVQLLQNGQ
KMPNQIQEVTKDNDWSYHFKDLPKYDAKNQEYKYSVEEVKVPDGYKVSYL
GNDIFNTRETEFVFEQNNFNLEFGNAEIKGQSGSKIIDEDTLTSFKGKKI
WKNDTAENRPQAIQVQLYADGVAVEGQTKFISGSGNEWSFEFKNLKKYNG
TGNDIIYSVKEVTVPTGYDVTYSANDIINTKREVITQQGPNLEIEETLPL
ESGASGGTTTVEDSRSVDTLSGLSSEQGQSGDMTIEEDSATHIKFSKRDI
DGKELAGATMELRDSSGKTISTWISDGQVKDFYLMPGKYTFVETAAPDGY
EIATAITFTVNEQGQVTVNGKATKGDAHIVMVDAYKPTKGSGQVIDIEEK
LPDEQGHSGSTTEIEDSKPSDVIIGGQGEVVDTTEDTQSGMTGHSGSTTE
IEDSKSSDVIIGGQGQVVETTEDTQTGMHGDSGCKTEVEDTKLVQFFHFD
NKEPESNSEIPKKDKPKSNTSLPATGEKQHNKFFWMVTSCSLISSVFVIS
LKSKKRLLSC

Two E boxes containing conserved glutamic residues have been identified in SpyoM01000149. The E-box motifs are underlined in SEQ ID NO:250, below. The conserved glutamic acid (E) residues, at amino acid residues 329 and 668, are marked in bold. The E box motifs, in particular the conserved glutamic acid residues, are thought to be important for the formation of oligomeric pilus-like structures of SpyoM01000149. Preferred fragments of SpyoM01000149 include at least one conserved glutamic acid residue. Preferably, fragments include at least one E box motif.

SEQ ID NO: 250

MTQKNSYKLSFLLSLTGFILGLLLVFIGLSGVSVGHAETRNGANKQGYFE
IKKVDQNNKPLSGATFSLTPKDGKGKPVQTFTSSEEGIIDAQNLQPGTYT
LKEETAPDGYDKTSRTWTVTVYENGYTKLVENPYNGEIISKAGSKDVSSS
LQLENPKMSVVSKYGEQEKTSNSADFYRNHAAYFKMSFELKQKDKSETIN
PGDTFVLQLDRRLNPKGISQDIPKIIYDSENSPLAIGKYDAKTHQLTYTF
TNYIAGLDKVQLSAELSLFLENKEVLENTNISDFKSTIGGQEITYKGTVN
VLYGNESTKESNYITNGLSNVGGSIESYNTETGEFVWYVYVNPNRTNIPY
AVLNLWGFAKRTAQGENDNSSVSSAQLTGYDIYEVPHNYRLPTSYGVDIS
RLNLRKDLEAKLPQGSTQGANKRLRIDFGENLQGKAFVVKVTGKADQSGK
ELIVQSHLSSFNNWGSYKTLRPNSHVSFTNEIALSPSKGSGSGTSEFTKP
AITVANLKRVAQLRFKKVSTDNVPLPEAAFELRSSNGNSQKLEASSNTQG
EIHFKDLTSGTYDLYETKAPKGYQQVTEKLATVTVDTTKPAEQMVKWEKP
HSFVKVEANKEVTIVNHKETLTFSGKKIWENDRPDQRPAKIQVQLLQNGQ
KMPNQIQEVTKDNDWSYHFKDLPKYDAKNQEYKYSVEEVKVPDGYKVSYL
GNDIFNTRETEFVFEQNNFNLEFGNAEIKGQSGSKIIDEDTLTSFKGKKI
WKNDTAENRPQAIQVQLYADGVAVEGQTKFISGSGNEWSFEFKNLKKYNG
TGNDIIYSVKEVTVPTGYDVTYSANDIINTKREVITQQGPNLEIEETLPL
ESGASGGTTTVEDSRSVDTLSGLSSEQGQSGDMTIEEDSATHIKFSKRDI
DGKELAGATMELRDSSGKTISTWISDGQVKDFYLMPGKYTFVETAAPDGY
EIATAITFTVNEQGQVTVNGKATKGDAHIVMVDAYKPTKGSGQVIDIEEK
LPDEQGHSGSTTEIEDSKPSDVIIGGQGEVVDTTEDTQSGMTGHSGSTTE
IEDSKSSDVIIGGQGQVVETTEDTQTGMHGDSGCKTEVEDTKLVQFFHFD
NKEPESNSEIPKKDKPKSNTSLPATGEKQHNKFFWMVTSCSLISSVFVIS
LKSKKRLLSC

As discussed above, applicants have also determined the nucleotide and encoded amino acid sequence of fimbrial structural subunits in several other GAS AI-3 strains of bacteria. Examples of sequences of these fimbrial structural subunits are set forth below.

M3 strain isolate ISS 3040 is a GAS AI-3 strain of bacteria. ISS3040_fimbrial is thought to be a fimbrial structural subunit of M3 strain isolate ISS 3040. An example of a nucleotide sequence encoding the ISS3040_fimbrial protein (SEQ ID NO:263) and an ISS3040_fimbrial protein amino acid sequence (SEQ ID NO:264) are set forth below.

SEQ ID NO: 263

GAGACGGCAGGAGTGTCCGAAAATGCAAAATTAATAGTAAAAAAGACATT
TGACTCTTATACAGACAATGAAGTTTTAATGCCAAAAGCTGATTATACTT
TTAAAGTAGAGGCAGATAGTACAGCTAGTGGCAAAACGAAAGACGGTTTA
GAGATTAAGCCAGGTATTGTTAATGGTTTAACAGAACAGATTATCAGCTA
TACTAATACTGATAAACCAGATAGTAAAGTTAAAAGTACAGAGTTTGATT
TTTCAAAAGTAGTATTCCCTGGTATTGGTGTTTACCGCTATACTGTTTCA
GAAAAACAAGGTGATGTTGAAGGAATTACCTACGATACTAAGAAGTGGAC
AGTAGATGTTTATGTTGGAAACAAAGAAGGTGGTGGTTTTGAACCTAAGT
TTATTGTATCTAAGGAACAAGGAACAGACGTCAAAAAACCAGTTAATTTT
AACAACTCGTTTGCAACTACTTCGTTAAAAGTTAAGAAGAATGTATCGGG
GAATACTGGAGAATTGCAAAAAGAATTTGACTTTACATTGACGCTTAATG
AAAGCACGAATTTTAAAAAAGATCAAATTGTTTCTTTACAAAAAGGAAAC
GAGAAATTTGAAGTTAAGATTGGTACTCCCTACAAGTTTAAACTCAAAAA
TGGGGAATCTATTCAACTAGACAAGTTACCAGTTGGTATTACTTATAAAG
TCAATGAAATGGAAGCTAATAAAGATGGGTATAAAACAACAGCATCCTTG
AAAGAGGGAGATGGTCAATCTAAAATGTATCAATTGGATATGGAACAAAA
AACAGACGAATCTGCTGACGAAATCGTTGTCACAAATAAGCGTGACACTC
AAGTTCCAACTGGTGTTGTAGGCACCCTTGCTCCATTTGCAGTTCTTAGC

SEQ ID NO: 264

ETAGVSENAKLIVKKTFDSYTDNEVLMPKADYTKFVEADSTASGKTKDGL
EIKPGIVNGLTEQIISYTNTDKPDSKVKSTEFDFSKVVFPGIGVYRYTVS
EKQGDVEGITYDTKKWTVDVYVGNKEGGGFEPKFIVSKEQGTDVKKPVNF
NNSFATTSLKVKKNVSGNTGELQKEFDFTLTLNESTNFKKDQIVSLQKGN
EKFEVKIGTPYKFKLKNGESIQLDKLPVGITYKVNEMEANKDGYKTTASL
KEGDGQSKMYQLDMEQKTDESADEIVVTNKRDTQVPTGVVGTLAPFAVLS

M44 strain isolate ISS 3776 is a GAS AI-3 strain of bacteria. ISS3776_fimbrial is thought to be a fimbrial structural subunit of M44 isolate ISS 3776. An example of a nucleotide sequence encoding the ISS3776_fimbrial protein (SEQ ID NO:253) and an ISS3776_fimbrial protein amino acid sequence (SEQ ID NO:254) are set forth below.

SEQ ID NO: 253

ttggagagagaaaaaatgaaaaaaaacaaattattacttgctactgcaat
cttagcaactgctttaggaacagcttctttaaatcaaaacgtaaaagctg
agacggcaggggttgtaacaggaaaatcactacaagttacaaagacaatg
acttatgatgatgaagaggtgttaatgcccgaaaccgcctttacttttac
tatagagcctgatatgactgcaagtggaaaagaaggcagcctagatatta
aaaatggaattgtagaaggcttagacaaacaagtaacagtaaaatataag
aatacagataaaacatctcaaaaaactaaaatagcacaatttgatttttc
taaggttaaatttccagctataggtgtttaccgctatatggtttcagaga
aaaacgataaaaaagacggaattacgtacgatgataaaaagtggactgta
gatgtttatgttgggaataaggccaataacgaagaaggtttcgaagttct
atatattgtatcaaaagaaggtacttctagtactaaaaaaccaattgaat
ttacaaactctattaaaactacttccttaaaaattgaaaaacaaataact
ggcaatgcaggagatcgtaaaaaatcattcaacttcacattaacattaca
accaagtgaatattataaaactggatcagttgtgaaaatcgaacaggatg
gaagtaaaaaagatgtgacgataggaacgccttacaaatttactttggga
cacggtaagagtgtcatgttatcgaaattaccaattggtatcaattacta
tcttagtgaagacgaagcgaataaagacggctacactacaacggcaacat
taaaagaacaaggcaaagaaaagagttccgatttcactttgagtactcaa
aaccagaaaacagacgaatctgctgacgaaatcgttgtcacaaataagcg
tgacactcaagttccaactggtgttgtagggacccttgctccatttgcag
ttcttagcattgtggctattggtggagttatctatattacaaaacgtaaa
aaagcttaa

SEQ ID NO: 254

MEREKMKKNKLLLATAILATALGTASLNQNVKAETAGVVTGKSLQVTKTM
TYDDEEVLMPETAFTFTIEPDMTASGKEGSLDIKNGIVEGLDKQVTVKYK
NTDKTSQKTKIAQFDFSKVKFPAIGVYRYMVSEKNDKKDGITYDDKKWTV
DVYVGNKANNEEGFEVLYIVSKEGTSSTKKPIEFTNSIKTTSLKIEKQIT
GNAGDRKKSFNFTLTLQPSEYYKTGSVVKIEQDGSKKDVTIGTPYKFTLG
HGKSVMLSKLPIGINYYLSEDEANKDGYTTTATLKEQGKEKSSDFTLSTQ
NQKTDESADEIVVTNKRDTQVPTGVVGTLAPFAVLSIVAIGGVIYITKRK
KA

M77 strain isolate ISS4959 is a GAS AI-3 strain of bacteria. ISS4959_fimbrial is thought to be a fimbrial structural subunit of M77 strain ISS 4959. An example of a nucleotide sequence encoding the ISS4959_fimbrial protein (SEQ ID NO:271) and an ISS4959_fimbrial protein amino acid sequence (SEQ ID NO:272) are set forth below.

SEQ ID NO: 271

gtaacagtaaaatataagaatacagataaaacatctcaaaaaactaaaat
agcacaatttgatttttctaaggttaaatttccagctataggtgtttacc
gctatatggtttcagagaaaaacgataaaaaagacggaattacgtacgat
gataaaaagtggacngtagatgtttatgttgggaataaggccaataacga
agaaggtttcgaagttctatatattgtatcaaaagaaggtacttctagtn
ctaaaaaaccaattgaatttacaaactctattaaaactacttccttaaaa
attgaaaaacaaataactggcaatgcaggagatcgtaaaaaatcattcaa
cttcacattnacattacanccaagtgaatattataaaactggatcagttg
tgaaaatcgaacaggatggaagtaaaaaagatgtgacgataggaacgcct
tacaaatttactttgggacacggtaagagtgtcatgttatcgaaattncc
aattggtatcaattactatcttagtgaagacgaagcgaataaagacggnt
acactacancggcaacattaaaagaacaaggcaaagaaaagagttccgat
ttcactttgagtactcaaaaccagaaaacagacgaatctgctg

SEQ ID NO: 272

VTVKYKNTDKTSQKTKIAQFDFSKVKFPAIGVYRYMVSEKNDKKDGITYD
DKKWTVDVYVGNKANNEEGFEVLYIVSKEGTSSXKKPIEFTNSIKTTSLK
IEKQITGNAGDRKKSFNFTXTLXPSEYYKTGSVVKIEQDGSKKDVTIGTP
YKFTLGHGKSVMLSKXPIGINYYLSEDEANKDGYTTXATLKEQGKEKSSD
FTLSTQNQKTDESA

Examples of GAS AI-4 sequences from M12 strain isolate A735 are set forth below.

19224133 is thought to be a RofA regulatory protein. An example of a nucleotide sequence encoding the RofA regulatory protein (SEQ ID NO:104) and a RofA regulatory protein amino acid sequence (SEQ ID NO:105) are set forth below.

SEQ ID NO: 104

ATGACCATCCAAAAAAGGATGATATCTTGCCAATTTACACATCCTTCTAA
AGAAACTTATCTTTACCAACTCTATGCATCATCTAATGTCTTACAATTAC
TAGCGTTTTTAATAAAAAATGGTTCCCACTCTCGTCCCCTTACGGATTTT
GCAAGAAGTCATTTTTTATCAAACTCCTCAGCTTATCGGATGCGCGAAGC
ATTGATTCCTTTATTAAGAAACTTTGAATTAAAACTCTCTAAGAACAAGA
TTGTCGGTGAGGAATATCGTATCCGTTACCTCATCGCTCTGCTATATAGT
AAGTTTGGCATTAAAGTTTATGACTTGACGCAGCAAGACAAAAACATTAT
TCATAGCTTTTTATCCCATAGTTCCACCCACCTTAAAACTTCTCCTTGGT
TATCGGAATCGTTTTCTTTCTATGACATTTTATTAGCTTTATCGTGGAAG
CGGCATCAATTTTCGGTAACTATTCCCCAAACCAGAATTTTTCAACAATT
AAAAAAACTTTTTGTCTACGATTCTTTGAAAAAAAGTAGCCGTGATATTA
TCGAAACTTACTGCCAACTAAACTTTTCAGCAGGAGATTTGGACTACCTC
TATTTAATTTATATCACCGCTAATAATTCTTTTGCGAGCTTACAATGGAC
ACCTGAGCATATCAGACAATGTTGTCAACTTTTTGAAGAAAATGATACTT
TTCGCCTGCTTTTAAATCCTATCATCACTCTTTTACCTAACCTAAAAGAG
CAAAAGGCTAGTTTAGTAAAAGCTCTTATGTTTTTTTCAAAATCATTCTT
GTTTAATCTGCAACATTTTATTCCTGAGACCAACTTATTCGTTTCTCCGT
ACTATAAAGGAAACCAAAAACTCTATACGTCCTTAAAGTTAATTGTCGAA
GAGTGGATGGCCAAACTTCCTGGTAAGCGTTACTTGAACCATAAGCATTT
TCATCTTTTTTGCCACTATGTCGAGCAAATTCTAAGAAATATCCAACCTC
CTTTAGTTGTTGTTTTCGTAGCCAGTAATTTTATCAATGCTCATCTCCTA
ACAGATTCTTTCCCAAGGTATTTCTCGGATAAAAGCATTGATTTTCATTC
CTATTATCTATTGCAAGATAATGTTTATCAAATTCCTGATTTAAAGCCAG
ATTTGGTCATCACTCACAGTCAACTGATTCCTTTTGTTCACCATGAACTT
ACAAAAGGAATTGCTGTTGCTGAAATATCTTTTGATGAATCGATTCTGTC
TATCCAAGAATTGATGTATCAAGTTAAAGAGGAAAAATTCCAAGCTGATT
TAACCAAACAATTAACATAA

SEQ ID NO: 105

MTIQKRMISCQFTHPSKETYLYQLYASSNVLQLLAFLIKNGSHSRPLTDF
ARSHFLSNSSAYRMREALIPLLRNFELKLSKNKIVGEEYRIRYLIALLYS
KFGIKVYDLTQQDKNIIHSFLSHSSTHLKTSPWLSESFSFYDILLALSWK
RHQFSVTIPQTRIFQQLKKLFVYDSLKKSSRDIIETYCQLNFSAGDLDYL
YLIYITANNSFASLQWTPEHIRQCCQLFEENDTFRLLLNPIITLLPNLKE
QKASLVKALMFFSKSFLFNLQHFIPETNLFVSPYYKGNQKLYTSLKLIVE
EWMAKLPGKRYLNHKHFHLFCHYVEQILRNIQPPLVVVFVASNFINAHLL
TDSFPRYFSDKSIDFHSYYLLQDNVYQIPDLKPDLVITHSQLIPFVHHEL
TKGIAVAEISFDESILSIQELMYQVKEEKFQADLTKQLT

19224134 is thought to be a protein F fibronectin binding protein. An example of a nucleotide sequence encoding the protein F fibronectin binding protein (SEQ ID NO:106) and a protein F fibronectin binding protein amino acid sequence (SEQ ID NO:107) are set forth below.

SEQ ID NO: 106

ATGGTAAGCTCATATATGTTTGCGAGAGGAGAGAAAATGAATAACAAAAT
GTTTTTGAACAAAGAAGCCGGTTTTTTGGTACACACAAAAAGAAAAAGGC
GATTTGCTGTCACTTTAGTGGGAGTCTTTTTTCTGCTTTTGGCATGTGCG
GGTGCTATCGGTTTTGGTCAAGTAGCCTATGCTGCGGATGAGAAGACTGT
GCCGAATTTTAAAAGCCCAGATCCAGATTATCCCTGGTATGGTTATGATT
CGTATAGAGGAATATTTGCAAGATATCACAATTTAAAAGTAAATCTAAAA
GGAAGTAAGGAGTATCAAGCGTATTGTTTTAACCTAACAAAATACTTTCC
TCGCCCCACTTATAGTACTACAAATAATTTTTACAAGAAAATTGATGGGA
GTGGATCAGCGTTCAAATCTTATGCAGCGAATCCTAGGGTTTTAGATGAG
AATTTAGATAAATTAGAAAAAAATATACTGAATGTAATTTATAATGGATA
TAAAAGTAATGCAAATGGTTTTATGAATGGTATAGAAGATCTTAATGCTA
TACTAGTAACTCAAAACGCTATTTGGTACTATTCAGATAGTGCTCCATTA
AATGATGTTAATAAAATGTGGGAAAGAGAGGTTCGGAATGGGGAGATTAG
TGAGTCACAAGTTACTTTAATGCGTGAGGCATTGAAAAAACTAATTGATC
CCAATTTAGAAGCTACTGCAGCTAATAAAATCCCATCAGGATATCGTTTA
AATATCTTTAAGTCTGAAAATGAAGATTACCAAAATCTTTTAAGTGCTGA
ATATGTACCTGATGATCCCCCTAAACCTGGTGATACGTCAGAACATAATC
CTAAAACTCCCGAGTTGGATGGCACTCCAATTCCCGAGGACCCAAAACGT
CCAGATGAGAGTTCAGAACCTGCGCTTCCCCCATTAATGCCAGAGCTAGA
TGGTGAAGAAGTCCCAGAAGTTCCAAGCGAGAGCTTAGAACCTGCGCTTC
CCCCATTGATGCCAGAGCTAGATGGTGAAGAAGTCCCAGAAGTTCCAAGC
GAGAGCTTAGAACCTGCGCTTCCCCCATTGATGCCAGAGCTAGATGGTGA
AGAAGTCCCAGAAGTTCCAAGCGAGAGCTTAGAACCTGCGCTTCCCCCAT
TAATGCCAGAGCTAGATGGTGAAGAAGTCCCAGAAGTTCCAAGCGAGAGC
TTAGAACCTGCGCTTCCCCCATTGATGCCAGAGTTAGATGGTGAAGAAGT
CCCTGAAAAACCTAGTGTTGACTTACCTATTGAAGTTCCTCGTTATGAGT
TTAACAATAAAGACCAGTCACCTCTAGCGGGTGAGTCTGGTGAGACGGAG
TATATTACCGAAGTCTATGGAAATCAACAGAACCCTGTTGATATTGATAA
AAAACTTCCGAATGAAACAGGTTTTTCAGGAAATATGGTTGAGACAGAAG
ATACGAAAGAGCCAGAAGTGTTGATGGGAGGTCAAAGTGAGTCTGTTGAA
TTTACTAAAGACACTCAAACAGGCATGAGTGGTCAAACAACTCCTCAGGT
TGAGACAGAAGATACGAAAGAGCCAGAAGTGTTGATGGGAGGTCAAAGTG
AGTCTGTTGAATTTACTAAAGACACTCAAACAGGCATGAGTGGTCAAACA
ACTCCTCAGGTTGAGACAGAAGATACGAAAGAGCCAGGAGTGTTGATGGG
AGGCCAAAGTGAGTCTGTTGAATTTACTAAAGACACTCAAACAGGCATGA
GTGGTCAAACAACTCCTCAGGTTGAGACAGAAGACACGAAAGAGCCAGGA
GTGTTGATGGGAGGTCAAAGTGAGTCTGTTGAATTTACTAAAGACACTCA
AACAGGCATGAGCGGTTTCAGTGAAACAGTGACCATTGTTGAAGATACGC
GTCCGAAGTTAGTGTTCCATTTTGACAATAATGAGCCCAAAGTGGAAGAG
AATCGGGAAAAGCCTACAAAAAATATAACACCTATCCTTCCTGCAACAGG
AGATATTGAGAATGTTTTGGCCTTTCTTGGAATCCTTATTTTGTCAGTAC
TTTCTATTTTTAGCCTTTTAAAAAACAAACAAAACAATAAAGTCTGA

SEQ ID NO: 107

MVSSYMFARGEKMNNKMFLNKEAGFLVHTKRKRRFAVTLVGVFFLLLACA
GAIGFGQVAYAADEKTVPNFKSPDPDYPWYGYDSYRGIFARYHNLKVNLK
GSKEYQAYCFNLTKYFPRPTYSTTNNFYKKIDGSGSAFKSYAANPRVLDE
NLDKLEKNILNVIYNGYKSNANGFMNGIEDLNAILVTQNAIWYYSDSAPL
NDVNKMWEREVRNGEISESQVTLMREALKKLIDPNLEATAANKIPSGYRL
NIFKSENEDYQNLLSAEYVPDDPPKPGDTSEHNPKTPELDGTPIPEDPKR
PDESSEPALPPLMPELDGEEVPEVPSESLEPALPPLMPELDGEEVPEVPS
ESLEPALPPLMPELDGEEVPEVPSESLEPALPPLMPELDGEEVPEVPSES
LEPALPPLMPELDGEEVPEKPSVDLPIEVPRYEFNNKDQSPLAGESGETE
YITEVYGNQQNPVDIDKKLPNETGFSGNMVETEDTKEPEVLMGGQSESVE
FTKDTQTGMSGQTTPQVETEDTKEPEVLMGGQSESVEFTKDTQTGMSGQT
TPQVETEDTKEPGVLMGGQSESVEFTKDTQTGMSGQTTPQVETEDTKEPG
VLMGGQSESVEFTKDTQTGMSGFSETVTIVEDTRPKLVFHFDNNEPKVEE
NREKPTKNITPILPATGDIENVLAFLGILILSVLSIFSLLKNKQNNKV

19224134 contains an amino acid motif indicative of a cell wall anchor: SEQ ID NO:181 LPATG (shown in italics in SEQ ID NO:107, above). In some recombinant host cell systems, it may be preferable to remove this motif to facilitate secretion of a recombinant 19224134 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in 19224134. The pilin motif sequence is underlined in SEQ ID NO:107, below. Conserved lysine (K) residues are also marked in bold, at amino acid residues 275, 285, and 299. The pilin sequence, in particular the conserved lysine residues, are thought to be important for the formation of oligomeric, pilus-like structures. Preferred fragments of 19224134 include at least one conserved lysine residue. Preferably, fragments include the pilin sequence.

SEQ ID NO: 107

MVSSYMFARGEKMNNKMFLNKEAGFLVHTKRKRRFAVTLVGVFFLLLACA
GAIGFGQVAYAADEKTVPNFKSPDPDYPWYGYDSYRGIFARYHNLKVNLK
GSKEYQAYCFNLTKYFPRPTYSTTNNFYKKIDGSGSAFKSYAANPRVLEN
LDKLEKNILNVIYNGYKSNANGFMNGIEDLNAILVTQNAIWYYSDSAPLN
DVNKMWEREVRNGEISESQVTLMREALKKLIDPNLEATAANKIPSGYRLN
IFKSENEDYQNLLSAEYVPDDPPKPGDTSEHNPKTPELDGTPIPEDPKRP
DESSEPALPPLMPELDGEEVPEVPSESLEPALPPLMPELDGEEVPEVPSE
SLEPALPPLMPELDGEEVPEVPSESLEPALPPLMPELDGEEVPEVPSESL
EPALPPLMPELDGEEVPEKPSVDLPIEVPRYEFNNKDQSPLAGESGETEY
ITEVYGNQQNPVDIDKKLPNETGFSGNMVETEDTKEPEVLMGGQSESVEF
TKDTQTGMSGQTTPQVETEDTKEPEVLMGGQSESVEFTKDTQTGMSGQTT
PQVETEDTKEPGVLMGGQSESVEFTKDTQTGMSGQTTPQVETEDTKEPGV
LMGGQSESVEFTKDTQTGMSGFSETVTIVEDTRFKLVFHFDNNEPKVEEN
REKPTKNITPILPATGDIENVLAFLGILILSVLSIFSLLKNKQNNKV

Two E boxes containing conserved glutamic residues have been identified in 19224134. The E-box motifs are underlined in SEQ ID NO:107, below. The conserved glutamic acid (E) residues, at amino acid residues 487 and 524, are marked in bold. The E box motifs, in particular the conserved glutamic acid residues, are thought to be important for the formation of oligomeric pilus-like structures of 19224134. Preferred fragments of 19224134 include at least one conserved glutamic acid residue. Preferably, fragments include at least one E box motif.

SEQ ID NO: 107

MVSSYMFARGEKMNNKMFLNKEAGFLVHTKRKRRFAVTLVGVFFLLLACA
GATGFGQVAYAADEKTVPNFKSPDPDYPWYGYDSYRGIFARYHNLKVNLK
GSKEYQAYCFNLTKYFPRPTYSTTNNFYKKIDGSGSAFKSYAANPRVLDE
NLDKLEKNILNVIYNGYKSNANGFMNGIEDLNAILVTQNAIWYYSDSAPL
NDVNKMWEREVRNGEISESQVTLMREALKKLIDPNLEATAANKIPSGYRL
NIFKSENEDYQNLLSAEYVPDDPPKPGDTSEHNPKTPELDGTPIPEDPKR
PDESSEPALPPLMPELDGEEVPEVPSESLEPALPPLMPELDGEEVPEVPS
ESLEPALPPLMPELDGEEVPEVPSESLEPALPPLMPELDGEEVPEVPSES
LEPALPPLMPELDGEEVPEKPSVDLPIEVPRYEFNNKDQSPLAGESGETE
YITEVYGNQQNPVDIDKKLPNETGFSGNMVETEDTKEPEVLMGGQSESVE
FTKDTQTGMSGQTTPQVETEDTKEPEVLMGGQSESVEFTKDTQTGMSGQT
TPQVETEDTKEPGVLMGGQSESVEFTKDTQTGMSGQTTPQVETEDTKEPG
VLMGGQSESVEFTKDTQTGMSGFSETVTIVEDTRPKLVFHFDNNEPKVEE
NREKPTKNITPTLPATGDIENVLAFLGILILSVLSTFSLLKNKQNNKV

19224135 is thought to be a capsular polysaccharide adhesin (Cpa) protein. An example of a nucleotide sequence encoding the Cpa protein (SEQ ID NO:108) and a Cpa protein amino acid sequence (SEQ ID NO:109) are set forth below.

SEQ ID NO: 108

ATGAATAACAAAAAATTGCAAAAGAAGCAAGATGCTCCTCGGGTATCAAA
CAGAAAGCCAAAACAATTAACTGTCACTTTAGTGGGAGTATTTTTAATGT
TTTTGACCTTGGTAAGTTCCATGAGAGGTGCTCAAAGCATATTTGGAGAG
GAAAAGAGAATTGAAGAAGTCAGTGTTCCTAAAATAAAAAGTCCAGATGA
TGCCTACCCTTGGTATGGCTATGATTCATATGACTCTAGTCATCCTTACT
ATGAACGTTTTAAAGTAGCACATGATTTAAGGGTTAATTTAAATGGAAGT
AAGAGCTACCAAGTATATTGCTTTAATATCAATTCTCATTATCCGAATAG
AAAAAATGCTTTTTCTAAACAATGGTTTAAGAGAGTTGATGGGACAGGTG
ATGTGTTCACAAATTATGCTCAGACACCTAAGATTCGTGGAGAATCATTG
AATAATAAACTTTTAAGTATTATGTACAACGCTTATCCTAAAAATGCTAA
TGGCTATATGGATAAGATAGAACCATTAAATGCTATTTTAGTAACTCAAC
AAGCTGTTTGGTACTATTCTGACAGTTCTTATGGTAATATAAAAACGTTA
TGGGCATCTGAGCTTAAAGACGGAAAAATAGATTTTGAACAAGTAAAATT
AATGCGTGAAGCTTACTCAAACTAATTAGTGATGATTTAGAAGAAACATC
TAAAAATAAGCTACCTCAAGGATCTAAACTGAATATTTTTGTTCCGCAAG
ATAAATCTGTTCAAAATTTATTAAGTGCAGAGTACGTGCCTGAATCCCCT
CCGGCACCAGGTCAGTCTCCAGAACCGCCAGTGCAAACAAAAAAAACATC
AGTCATTATCAGAAAATATGCGGAAGGTGACTACTCTAAACTTCTAGAGG
GAGCAACTTTGCGTTTAACAGGGGAAGATATCCTAGATTTTCAAGAAAAA
GTCTTCCAAAGTAATGGAACAGGAGAAAAGATTGAATTATCAAATGGGAC
TTATACCTTAACAGAAACATCATCTCCAGATGGATATAAAATTGCGGAGC
CGATTAAGTTTAGAGTAGTGAATAAAAAAGTATTTATCGTCCAAAAAGAT
GGTTCTCAAGTGGAAAATCCAAACAAAGAAGTAGCAGAGCCATACTCAGT
GGAAGCGTACAGCGATATGCAAGATAGTAACTATATTAATCCAGAAACGT
TCACTCCTTATGGGAAATTTTATTACGCTAAAAATAAGGATAAAAGTTCA
CAAGTTGTCTACTGTTTTAATGCTGATTTACACTCTCCACCTGAATCAGA
GGATGGGGGAGGAACTATAGATCCTGATATTAGTACGATGAAAGAAGTCA
AGTACACACATACGGCAGGTAGTGATTTGTTTAAATACGCGCTAAGACCG
AGAGATACAAATCCAGAAGACTTCTTAAAGCACATTAAAAAAGTAATTGA
AAAAGGCTACAATAAAAAAGGTGATAGCTATAATGGATTAACAGAAACAC
AGTTTCGCGCGGCTACTCAGCTTGCTATCTATTACTTTACAGACAGCACT
GACTTAAAAACCTTAAAAACTTATAACAATGGGAAAGGTTACCATGGATT
TGAATCTATGGATGAAAAAACCCTAGCTGTAACAAAGAATTAATTAATTA
CGCTCAAGATAATAGTGCCCCTCAACTAACAAATCTTGATTTCTTCGTAC
CTAATAATAGCAAATACCAATCTCTTATTGGGACAGAATACCATCCAGAT
GATTTGGTTGACGTGATTCGTATGGAAGATAAAAAGCAAGAAGTTATTCC
AGTAACTCACAGTTTGACAGTGAAAAAAACAGTAGTCGGTGAGTTGGGAG
ATAAAACTAAAGGCTTCCAATTTGAACTTGAGTTGAAAGATAAAACTGGA
CAGCCTATTGTTAACACTCTAAAAACTAATAATCAAGATTTAGTAGCTAA
AGATGGGAAATATTCATTTAATCTAAAGCATGGTGACACCATAAGAATAG
AAGGATTACCGACGGGATATTCTTATACTCTGAAAGAGACTGAAGCTAAG
GATTATATAGTAACCGTTGATAACAAAGTTAGTCAAGAAGCTCAATCAGC
AAGTGAGAATGTCACAGCAGACAAAGAAGTCACTTTTGAAAACCGTAAAG
ATCTTGTCCCACCAACTGGTTTTATTACTGATGGTGGAACCTATCTGTGG
TTATTATTGCTTGTCCCATTTGGTTTGTTAGTGTGGTTCTTTGGTCGTAA
AGGACTAAAAAATGACTAA

SEQ ID NO: 109

MNNKKLQKKQDAPRVSNRKPKQLTVTLVGVFLMFLTLVSSMRGAQSIFGE
EKRIEEVSVPKIKSPDDAYPWYGYDSYDSSHPYYERFKVAHDLRVNLNGS
KSYQVYCFNINSHYPNRKNAFSKQWFKRVDGTGDVFTNYAQTPKIRGESL
NNKLLSIMYNAYPKNANGYMDKIEPLNAILVTQQAVWYYSDSSYGNIKTL
WASELKDGKIDFEQVKLMREAYSKLISDDLEETSKNKLPQGSKLNIFVPQ
DKSVQNLLSAEYVPESPPAPGQSPEPPVQTKKTSVIIRKYAEGDYSKLLE
GATLRLTGEDILDFQEKVFQSNGTGEKIELSNGTYTLTETSSPDGYKIAE
PIKFRVVNKKVFIVQKDGSQVENPNKEVAEPYSVEAYSDMQDSNYINPET
FTPYGKFYYAKNKDKSSQVVYCFNADLHSPPESEDGGGTIDPDISTMKEV
KYTHTAGSDLFKYALRPRDTNPEDFLKHIKKVIEKGYNKKGDSYNGLTET
QFRAATQLAIYYFTDSTDLKTLKTYNNGKGYHGFESMDEKTLAVTKELIN
YAQDNSAPQLTNLDFFVPNNSKYQSLIGTEYHPDDLVDVIRMEDKKQEVI
PVTHSLTVKKTVVGELGDKTKGFQFELELKDKTGQPIVNTLKTNNQDLVA
KDGKYSFNLKHGDTIRIEGLPTGYSYTLKETEAKDYIVTVDNKVSQEAQS
ASENVTADKEVTFENRKDLVPPTGFITDGGTYLWLLLLVPFGLLVWFFGR
KGLKND

19224135 contains an amino acid motif indicative of a cell wall anchor: SEQ ID NO:184 VPPTG (shown in italics in SEQ ID NO:109, above). In some recombinant host cell systems, it may be preferable to remove this motif to facilitate secretion of a recombinant 19224135 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in 19224135. The pilin motif sequence is underlined in SEQ ID NO:109, below. Conserved lysine (K) residues are also marked in bold, at amino acid residues 164 and 172. The pilin sequence, in particular the conserved lysine residues, are thought to be important for the formation of oligomeric, pilus-like structures. Preferred fragments of 19224135 include at least one conserved lysine residue. Preferably, fragments include the pilin sequence.

SEQ ID NO: 109

MNNKKLQKKQDAPRVSNRKPKQLTVTLVGVFLMFLTLVSSMRGAQSIFGE
EKRIEEVSVPKIKSPDDAYPWYGYDSYDSSHPYYERFKVAHDLRVNLNGS
KSYQVYCFNINSHYPNRKNAFSKQWFKRVDGTGDVFTNYAQTPKIRGESL
NNKLLSIMYNAYPKNANGYMDKIEPLNAILVTQQAVWYYSDSSYGNIKTL
WASELKDGKIDFEQVKLMREAYSKLISDDLEETSKNKLPQGSKLNIFVPQ
DKSVQNLLSAEYVPESPPAPGQSPEPPVQTKKTSVIIRKYAEGDYSKLLE
GATLRLTGEDILDFQEKVFQSNGTGEKIELSNGTYTLTETSSPDGYKIAE
PIKFRVVNKKVFIVQKDGSQVENPNKEVAEPYSVEAYSDMQDSNYINPET
FTPYGKFYYAKNKDKSSQVVYCFNADLHSPPESEDGGGTIDPDISTMKEV
KYTHTAGSDLFKYALRPRDTNPEDFLKHIKKVIEKGYNKKGDSYNGLTET
QFRAATQLAIYYFTDSTDLKTLKTYNNGKGYHGFESMDEKTLAVTKELIN
YAQDNSAPQLTNLDFFVPNNSKYQSLIGTEYHPDDLVDVIRMEDKKQEVI
PVTHSLTVKKTVVGELGDKTKGFQFELELKDKTGQPIVNTLKTNNQDLVA
KDGKYSFNLKHGDTIRIEGLPTGYSYTLKETEAKDYIVTVDNKVSQEAQS
ASENVTADKEVTFENRKDLVPPTGFITDGGTYLWLLLLVPFGLLVWFFGR
KGLKND

An E box containing a conserved glutamic residue has been identified in 19224135. The E-box motif is underlined in SEQ ID NO:109, below. The conserved glutamic acid (E), at amino acid residue 339, is marked in bold. The E box motif, in particular the conserved glutamic acid residue, is thought to be important for the formation of oligomeric pilus-like structures of 19224135. Preferred fragments of 19224135 include the conserved glutamic acid residue. Preferably, fragments include the E box motif.

SEQ ID NO: 109

MNNKKLQKKQDAPRVSNRKPKQLTVTLVGVFLMFLTLVSSMRGAQSIFGE
EKRIEEVSVPKIKSPDDAYPWYGYDSYDSSHPYYERFKVAHDLRVNLNGS
KSYQVYCFNINSHYPNRKNAFSKQWFKRVDGTGDVFTNYAQTPKIRGESL
NNKLLSIMYNAYPKNANGYMDKIEPLNAILVTQQAVWYYSDSSYGNIKTL
WASELKDGKIDFEQVKLMREAYSKLISDDLEETSKNKLPQGSKLNIFVPQ
DKSVQNLLSAEYVPESPPAPGQSPEPPVQTKKTSVIIRKYAEGDYSKLLE
GATLRLTGEDILDFQEKVFQSNGTGEKIELSNGTYTLTETSSPDGYKIAE
PIKFRVVNKKVFIVQKDGSQVENPNKEVAEPYSVEAYSDMQDSNYINPET
FTPYGKFYYAKNKDKSSQVVYCFNADLHSPPESEDGGGTIDPDISTMKEV
KYTHTAGSDLFKYALRPRDTNPEDFLKHIKKVIEKGYNKKGDSYNGLTET
QFRAATQLAIYYFTDSTDLKTLKTYNNGKGYHGFESMDEKTLAVTKELIN
YAQDNSAPQLTNLDFFVPNNSKYQSLIGTEYHPDDLVDVIRMEDKKQEVI
PVTHSLTVKKTVVGELGDKTKGFQFELELKDKTGQPIVNTLKTNNQDLVA
KDGKYSFNLKHGDTIRIEGLPTGYSYTLKETEAKDYIVTVDNKVSQEAQS
ASENVTADKEVTFENRKDLVPPTGFITDGGTYLWLLLLVPFGLLVWFFGR
KGLKND

19224136 is thought to be a LepA protein. An example of a nucleotide sequence encoding the LepA protein (SEQ ID NO:110) and a LepA protein amino acid sequence (SEQ ID NO:111) are set forth below.

SEQ ID NO: 110

ATGACTAATTACCTAAATCGCTTAAATGAGAATCCACTATTTAAAGCTTT
CATACGGTTAGTACTTAAGATTTCTATTATTGGATTTCTAGGTTACATTC
TATTTCAGTATGTTTTTGGCGTCATGATTGTTAACACAAATCAGATGAGT
CCTGCTGTAAGTGCTGGTGATGGAGTCTTATATTATCGTTTGACTGATCG
CTATCATATTAATGATGTGGTGGTCTATGAGGTTGATAACACTTTGAAAG
TTGGTCGAATTGCCGCTCAAGCTGGCGATGAGGTTAGTTTTACGCAAGAA
GGAGGACTGTTGATTAATGGGCATCCACCAGAAAAAGAGGTCCCTTACCT
GACGTATCCTCACTCAAGTGGTCCAAACTTTCCCTATAAAGTTCCTACGG
GTACGTATTTCATATTGAATGATTATCGTGAAGAACGTTTGGACAGTCGT
TATTATGGGGCGTTACCCATCAATCAAATCAAAGGGAAAATCTCAACTCT
ATTAAGAGTGAGAGGAATTTAA

SEQ ID NO: 111

MTNYLNRLNENPLFKAFIRLVLKTSIIGFLGYILFQYVFGVMIVNTNQMS
PAVSAGDGVLYYRLTDRYHINDVVVYEVDNTLKVGRIAAQAGDEVSFTQE
GGLLINGHPPEKEVPYLTYPHSSGPNFPYKVPTGTYFILNDYREERLDSR
YYGALPINQIKOKISTLLRVRGI

19224137 is thought to be a fimbrial protein. An example of a nucleotide sequence encoding the fimbrial protein (SEQ ID NO:112) and a fimbrial protein amino acid sequence (SEQ ID NO:113) are set forth below.

SEQ ID NO: 112

ATGAAAAAAAATAAATTATTACTTGCTACTGCAATCTTAGCAACTGCTTT
AGGAACAGCTTCTTTAAATCAAAACGTAAAAGCTGAGACGGCAGGGGTTG
TTAGCAGTGGTCAATTAACAATAAAAAAATCAATTACAAATTTTAATGAT
GATACACTTTTGATGCCTAAGACAGACTATACTTTTAGCGTTAATCCGGA
TAGTGCGGCTACAGGTACTGAAAGTAATTTACCAATTAAACCAGGTATTG
CTGTTAACAATCAAGATATTAAGGTTTCTTATTCTAATACTGATAAGACA
TCAGGTAAAGAAAAACAAGTTGTTGTTGACTTTATGAAAGTTACTTTTCC
TAGCGTTGGTATTTACCGTTATGTTGTTACCGAGAATAAAGGGACAGCAG
AAGGAGTTACATATGATGATACAAAATGGTTAGTTGACGTCTATGTTGGT
AATAATGAAAAGGGAGGTCTTGAACCAAAGTATATTGTATCTAAAAAAGG
AGATTCTGCTACTAAAGAACCAATCCAGTTTAATAATTCATTCGAAACAA
CGTCATTAAAAATTGAAAAGGAAGTTACTGGTAATACAGGAGATCATAAA
AAAGCATTTACCTTTACATTAACATTGCAACCAAATGAATACTATGAGGC
AAGTTCGGTTGTGAAAATTGAAGAGAACGGACAAACGAAAGATGTGAAAA
TTGGGGAGGCATATAAGTTTACTTTGAACGATAGTCAGAGTGTGATATTG
TCTAAATTACCAGTTGGTATTAATTATAAAGTTGAAGAAGCAGAAGCTAA
TCAAGGTGGATATACTACAACAGCAACTTTAAAAGATGGAGAAAAGTTAT
CTACTTATAACTTAGGTCAGGAACATAAAACAGACAAGACTGCTGATGAA
ATCGTTGTCACAAATAACCGTGACACTCAAGTTCCAACTGGTGTTGTAGG
CACCCTTGCTCCATTTGCAGTTCTTAGCATTGTGGCTATTGGTGGAGTTA
TCTATATTACAAAACGTAAAAAAGCTTAA

SEQ ID NO: 113

MKKNKLLLATAILATALGTASLNQNVKAETAGVVSSGQLTIKKSITNFND
DTLLMPKTDYTFSVNPDSAATGTESNLPIKPGIAVNNQDIKVSYSNTDKT
SGKEKQVVVDFMKVTFPSVGIYRYVVTENKGTAEGVTYDDTKWLVDVYVG
NNEKGGLEPKYIVSKKGDSATKEPIQFNNSFETTSLKIEKEVTGNTGDHK
KAFTFTLTLQPNEYYEASSVVKIEENGQTKDVKIGEAYKFTLNDSQSVIL
SKLPVGINYKVEEAEANQGGYTTTATLKDGEKLSTYNLGQEHKTDKTADE
IVVTNNRDTQVPTGVVGTLAPFAVLSIVAIGGVIYITKRKKA

19224137 contains an amino acid motif indicative of a cell wall anchor: SEQ ID NO:140 QVPTG (shown in italics in SEQ ID NO:113, above). In some recombinant host cell systems, it may be preferable to remove this motif to facilitate secretion of a recombinant 19224137 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in 19224137. The pilin motif sequence is underlined in SEQ ID NO:113, below. A conserved lysine (K) residue is also marked in bold, at amino acid residue 160. The pilin sequence, in particular the conserved lysine residues, are thought to be important for the formation of oligomeric, pilus-like structures. Preferred fragments of 19224137 include the conserved lysine residue. Preferably, fragments include the pilin sequence.

SEQ ID NO: 113

MKKNKLLLATAILATALGTASLNQNVKAETAGVVSSGQLTIKKSITNFND
DTLLMPKTDYTFSVNPDSAATGTESNLPIKPGIAVNNQDIKVSYSNTDKT
SGKEKQVVVDFMKVTFPSVGIYRYVVTENKGTAEGVTYDDTKWLVDVYVG
NNEKGGLEPKYIVSKKGDSATKEPIQFNNSFETTSLKIEKEVTGNTGDHK
KAFTFTLTLQPNEYYEASSVVKIEENGQTKDVKIGEAYKFTLNDSQSVIL
SKLPVGTNYKVEEAEANQGGYTTTATLKDGEKLSTYNLGQEHKTDKTADE
IVVTNNRDTQVPTGVVGTLAPFAVLSIVAIGGVIYITKRKKA

An E box containing a conserved glutamic residue has been identified in 19224137. The E-box motif is underlined in SEQ ID NO:113, below. The conserved glutamic acid (E), at amino acid residue 263, is marked in bold. The E box motif, in particular the conserved glutamic acid residue, is thought to be important for the formation of oligomeric pilus-like structures of 19224137. Preferred fragments of 19224137 include the conserved glutamic acid residue. Preferably, fragments include the E box motif.

SEQ ID NO: 113

MKKNKLLLATAILATALGTASLNQNVKAETAGVVSSGQLTIKKSITNFND
DTLLMPKTDYTFSVNPDSAATGTESNLPIKPGIAVNNQDIKVSYSNTDKT
SGKEKQVVVDFMKVTFPSVGTYRYVVTENKGTAEGVTYDDTKWLVDVYVG
NNEKGGLEPKYIVSKKGDSATKEPIQFNNSFETTSLKIEKEVTGNTGDHK
KAFTFTLTLQPNEYYEASSVVKIEENGQTKDVKIGEAYKFTLNDSQSVIL
SKLPVGINYKVEEAEANQGGYTTTATLKDGEKLSTYNLGQEHKTDKTADE
IVVTNNRDTQVPTGVVGTLAPFAVLSIVAIGGVIYITKRKKA

19224138 is thought to be a SrtC2-type sortase. An example of a nucleotide sequence encoding the SrtC2 sortase (SEQ ID NO:114) and a SrtC2 sortase amino acid sequence (SEQ ID NO:115) are set forth below.

SEQ ID NO: 114

ATGATGATGACAATTGTACAGGTTATCAATAAAGCCATTGATACTCTCAT
TCTTATCTTTTGTTTAGTCGTACTATTTTTAGCTGGTTTTGGTTTGTGGG
ATTCTTATCATCTCTATCAACAAGCAGACGCTTCTAATTTCAAAAAATTT
AAAACAGCTCAACAACAGCCTAAATTTGAAGACTTGTTAGCTTTGAATGA
GGATGTCATTGGTTGGTTAAATATCCCGGGGACTCATATTGATTATCCTC
TAGTTCAGGGAAAAACGAATTTAGAGTATATTAATAAAGCAGTTGATGGC
AGTGTTGCCATGTCTGGTAGTTTATTTTTAGATACACGGAATCATAATGA
TTTTACGGACGATTACTCTCTGATTTATGGCCATCATATGGCAGGTAATG
CCATGTTTGGCGAAATTCCAAAATTTTTAAAAAAGGATTTTTTCAACAAA
CATAATAAAGCTATCATTGAAACAAAAGAGAGAAAAAAACTAACCGTCAC
TATTTTTGCTTGTCTCAAGACAGATGCCTTTGACCAGTTAGTTTTTAATC
CTAATGCTATTACCAATCAAGACCAACAAAGGCAGCTCGTTGATTATATC
AGTAAAAGATCAAAACAATTTAAACCTGTTAAATTGAAGCATCATACAAA
GTTCGTTGCTTTTTCAACGTGTGAAAATTTTTCTACTGACAATCGTGTTA
TCGTTGTCGGTACTATTCAAGAATAA

SEQ ID NO: 115

MMMTIVQVINKATDTLILIFCLVVLFLAGFGLWDSYHLYQQADASNFKKF
KTAQQQPKFEDLLALNEDVIGWLNIPGTHIDYPLVQGKTNLEYINKAVDG
SVAMSGSLFLDTRNHNDFTDDYSLIYGHHMAGNANFGEIPKFLKKDFFNK
HNKAIIETKERKKLTVTIFACLKTDAFDQLVFNPNAITNQDQQRQLVDYI
SKRSKQFKPVKLKHHTKFVAFSTCENFSTDNRVIVVGTIQE

19224139 is an open reading frame that encodes a sortase substrate motif LPXAG shown in italics in SEQ ID NO:117. An example of a nucleotide sequence of the open reading frame (SEQ ID NO:116) and the amino acid sequence encoded by the open reading frame (SEQ ID NO:117) are set forth below.

SEQ ID NO: 116

ATGTTATTTTCTGTCGTAATGATATTAACCATGCTGGCCTTTAATCAGAC
TGTTTTAGCAAAAGACAGCACTGTTCAAACTAGCATTAGTGTCGAAAATG
TCTTAGAGAGAGCAGGCGATAGTACCCCATTTTCGATTGCATTAGAATCA
ATTGATGCGATGAAAACAATAGAAGAAATAACAATTGCTGGTTCTGGAAA
AGCAAGCTTTTCCCCTCTGACCTTCACAACAGTTGGGCAATATACTTATC
GTGTTTATCAGAAGCCTTCACAAAATAAAGATTATCAAGCAGATACTACT
GTATTTGACGTTCTTGTCTATGTGACCTATGATGAAGATGGGACTCTAGT
CGCAAAAGTTATTTCTCGAAGGGCTGGAGACGAAGAAAAATCAGCGATTA
CTTTTAAGCCCAAACGGTTAGTAAAACCAATACCGCCTAGACAACCTAAC
ATCCCTAAAACCCCATTACCATTAGCTGGTGAAGTAAAAAGTTTATTGGG
TATCTTAAGTATCGTATTACTGGGGTTACTAGTTCTTCTTTATGTTAAAA
AACTGAAGAG

SEQ ID NO: 117

MLFSVVMTLIMLAFNQTVLAKDSTVQTSISVENVLERAGDSTPFSIALES
IDAMKTIEEITIAGSGKASFSPLTFTTVGQYTYRVYQKPSQNKDYQADTT
VFDVLVYVTYDEDGTLVAKVISRPAGDEEKSAITFKPKRLVKPIPPRQPN
IPKTPLPLAGEVKSLLGILSIVLLGLLVLLYVKKLKSKL

19224139 contains an amino acid motif indicative of a cell wall anchor: SEQ ID NO:185 LPLAG (shown in italics in SEQ ID NO:117, above). In some recombinant host cell systems, it may be preferable to remove this motif to facilitate secretion of a recombinant 19224139 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A pilin motif, discussed above, containing a conserved lysine (K) residue has also been identified in 19224139. The pilin motif sequence is underlined in SEQ ID NO:117, below. A conserved lysine (K) residue is also marked in bold, at amino acid residue 138. The pilin sequence, in particular the conserved lysine residue, is thought to be important for the formation of oligomeric, pilus-like structures. Preferred fragments of 19224139 include the conserved lysine residue. Preferably, fragments include the pilin sequence.

SEQ ID NO: 117

MLFSVVMILTMLAFNQTVLAKDSTVQTSISVENVLERAGDSTPFSIALES
IDAMKTIEEITIAGSGKASFSPLTFTTVGQYTYRVYQKPSQNKDYQADTT
VFDVLVYVTYDEDGTLVAKVISRRAGDEEKSAITFKPKRLVKPIPPRQPN
IPKTPLPLAGEVKSLLGILSIVLLGLLVLLYVKKLKSKL

Two E boxes containing conserved glutamic residues have been identified in 19224139. The E-box motifs are underlined in SEQ ID NO:117, below. The conserved glutamic acid (E) residues, at amino acid residues 58 and 128, are marked in bold. The E box motifs, in particular the conserved glutamic acid residues, are thought to be important for the formation of oligomeric pilus-like structures of 19224139. Preferred fragments of 19224139 include at least one conserved glutamic acid residue. Preferably, fragments include at least one E box motif.

SEQ ID NO: 117

MLFSVVMILTMLAFNQTVLAKDSTVQTSISVENVLERAGDSTPFSIA
LESIDAMKTIEEITIAGSGKASFSPLTFTTVGQYTYRVYQKPSQNKD
YQADTTVFDVLVYVTYDEDGTLVAKVISRRAGDEEKSAITFKPKRLV
KPIPPRQPNIPKTPLPLAGEVKSLLGILSIVLLGLLVLLYVKKLKSK
L

19224140 is thought to be a MsmRL protein. An example of a nucleotide sequence encoding the MsmRL protein (SEQ ID NO:118) and a MsmRL protein amino acid sequence (SEQ ID NO:119) are set forth below.

SEQ ID NO: 118

ATGGTTATATTCGATTTAAAACATGTGCAAACATTACACAGCTTGTCTCA
ATTACCTATTTCAGTGATGTCACAAGATAAGGCACTTATTCAAGTATATG
GTAATGACGACTATTTATTATGTTACTATCAATTTTTAAAGCATCTAGCT
ATTCCTCAAGCTGCACAAGATGTTATTTTTTATGAGGGTTTATTTGAAGA
GTCCTTTATGATTTTTCCTCTTTGTCACTACATTATTGCCATTGGACCTT
TCTACCCTTATTCACTTAATAAAGACTATCAGGAACAATTAGCTAATAAT
TTTTTAAAACATTCTTCTCATCGTAGCAAAGAAGAGCTCTTATCCTATAT
GGCACTTGTCCCACATTTTCCAATTAATAATGTGCGGAACCTTTTGATAG
CTATTGACGCTTTTTTTGACACACAATTTGAGACGACTTGCCAACAAACA
ATTCATCAATTGTTGCAGCATTCAAAACAGATGACTGCTGATCCTGATAT
CATTCATCGCCTTAAGCATATTAGCAAAGCATCTAGCCAACTACCGCCTG
TTTTAGAGCACCTAAATCATATTATGGATCTGGTAAAGCTAGGCAATCCA
CAATTGCTCAAGCAAGAAATCAATCGCATCCCCTTATCAAGTATCACCTC
ATCTTCTATTTCTGCTCTAAGGGCGGAAAAGAACCTCACTGTTATCTATT
TAACTAGGTTACTGGAATTCAGTTTTGTAGAAAATACTGACGTAGCAAAG
CATTATAGCCTTGTCAAATACTACATGGCCTTAAATGAAGAAGCGAGTGA
CTTGCTCAAAGTTTTGAGAATTCGCTGTGCAGCCATCATCCATTTTTCCG
AATCATTAACCAATAAAAGTATTTCTGATAAACGTCAAATGTACAATAGT
GTGCTTCATTATGTCGATAGTCACCTGTATTCCAAATTAAAGGTATCTGA
TATCGCTAAGCGCCTATATGTTTCCGAATCTCACTTACGTTCAGTCTTTA
AAAAATACTCAAATGTTTCCTTACAACATTATATTCTAAGTACAAAAATC
AAAGAAGCTCAACTACTCTTAAAACGAGGAATTCCTGTTGGAGAAGTGGC
TAAAAGCTTATATTTTTATGACACTACCCATTTTCATAAAATCTTTAAAA
AATACACGGGTATTTCTTCAAAAGACTATCTTGCTAAATACCGAGATAAT
ATTTAA

SEQ ID NO: 119

MVIFDLKHVQTLHSLSQLPISVMSQDKALIQVYGNDDYLLCYYQFLKHLA
IPQAAQDVIFYEGLFEESFMIFPLCHYIIAIGPFYPYSLNKDYQEQLANN
FLKHSSHRSKEELLSYMALVPHFPINNVRNLLIAIDAFFDTQFETTCQQT
IHQLLQHSKQMTADPDIIHRLKHISKASSQLPPVLEHLNHIMDLVKLGNP
QLLKQEINRIPLSSITSSSISALPAEKNLTVIYLTRLLEFSFVENTDVAK
HYSLVKYYMALNEEASDLLKVLRIRCAAIIHFSESLTNKSISDKRQMYNS
VLHYVDSHLYSKLKVSDIAKRLYVSESHLRSVFKKYSNVSLQHYILSTKI
KEAQLLLKRGIPVGEVAKSLYFYDTTHFHKIFKKYTGISSKDYLAKYRDN
I

19224141 is thought to be a protein F2 fibronectin binding protein. An example of a nucleotide sequence encoding the protein F2 fibronectin binding protein (SEQ ID NO:120) and a protein F2 fibronectin binding protein amino acid sequence (SEQ ID NO:121) are set forth below.

SEQ ID NO: 120

ATGACACAAAAAAATAGCTATAAGTTAAGCTTCCTGTTATCCCTAACAGG
ATTTATTTTAGGTTTATTATTGGTTTTTATAGGATTGTCCGGAGTATCAG
TAGGACATGCGGAAACAAGAAATGGAGCAAACAAACAAGGATCTTTTGAA
ATCAAGAAAGTCGACCAAAACAATAAGCCTTTACCGGGAGCAACTTTTTC
ACTGACATCAAAGGATGGCAAGGGAACATCTGTTCAAACGTTCACTTCAA
ATGATAAAGGTATTGTAGATGCTCAAAATCTCCAACCAGGGACTTATACC
TTAAAAGAAGAAACAGCACCAGATGGTTATGATAAAACCAGCCGGACTTG
GACAGTGACTGTTTATGAGAACGGCTATACCAAGTTGGTTGAAAATCCCT
ATAATGGGGAAATCATCAGTAAAGCAGGGTCAAAAGATGTTAGTAGTTCT
TTACAGTTGGAAAATCCCAAAATGTCAGTTGTTTCTAAATATGGGAAAAC
AGAGGTTAGTAGTGGCGCAGCGGATTTCTACCGCAACCATGCCGCCTATT
TTAAAATGTCTTTTGAGTTGAAACAAAAGGATAAATCTGAAACAATCAAC
CCAGGTGATACCTTTGTGTTACAGCTGGATAGACGTCTCAATCCTAAAGG
TATCAGTCAAGATATCCCTAAAATCATTTACGACAGTGCAAATAGTCCGC
TTGCGATTGGAAAATACCATGCTGAGAACCATCAACTTATCTATACTTTC
ACAGATTATATTGCGGGTTTAGATAAAGTCCAGTTGTCTGCAGAATTGAG
CTTATTCCTAGAGAATAAGGAAGTGTTGGAAAATACTAGTATCTCAAATT
TTAAGAGTACCATAGGTGGGCAGGAGATCACCTATAAAGGAACGGTTAAT
GTTCTTTATGGAAATGAGAGCACTAAAGAAAGCAATTATATTACTAATGG
ATTGAGCAATGTGGGTGGGAGTATTGAAAGCTACAACACCGAAACGGGAG
AATTTGTCTGGTATGTTTATGTCAATCCAAACCGTACCAATATTCCTTAT
GCGACCATGAATTTATGGGGATTTGGAAGGGCTCGTTCAAATACAAGCGA
CTTAGAAAACGACGCTAATACAAGTAGTGCTGAGCTTGGAGAGATTCAGG
TCTATGAAGTACCTGAAGGAGAAAAATTACCATCAAGTTATGGGGTTGAT
GTTACAAAACTTACTTTAAGAACGGATATCACAGCAGGCCTAGGAAATGG
TTTTCAAATGACCAAACGTCAGCGAATTGACTTTGGAAATAATATCCAAA
ATAAAGCATTTATCATCAAAGTAACAGGGAAAACAGACCAATCTGGTAAG
CCATTGGTTGTTCAATCCAATTTGGCAAGTTTTCGTGGTGCTTCTGAATA
TGCTGCTTTTACTCCAGTTGGAGGAAATGTCTACTTCCAAAACGAAATTG
CCTTGTCTCCTTCTAAGGGTAGTGGTTCTGGGAAAAGTGAATTTACTAAG
CCCTCTATTACAGTAGCAAATCTAAAACGAGTGGCTCAGCTTCGCTTTAA
GAAAATGTCAACTGACAATGTGCCATTGCCAGAAGCGGCTTTTGAGCTGC
GTTCATCAAATGGTAATAGTCAGAAATTAGAAGCCAGTTCAAACACACAA
GGAGAGGTTCACTTTAAGGACCTGACCTCGGGCACATATGACCTGTATGA
AACAAAAGCGCCAAAAGGTTATCAGCAGGTGACAGAGAAATTGGCGACCG
TTACTGTTGATACTACCAAACCTGCTGAGGAAATGGTCACTTGGGGAAGC
CCACATTCGTCTGTAAAAGTAGAAGCTAACAAAGAAGTCACGATTGTCAA
CCATAAAGAAACCCTTACGTTTTCAGGGAAGAAAATTTGGGAGAATGACA
GACCAGATCAACGCCCAGCAAAGATTCAAGTGCAACTGTTGCAAAATGGT
CAAAAGATGCCTAACCAGATTCAAGAAGTAACGAAGGATAACGATTGGTC
TTATCACTTCAAAGACTTGCCTAAGTACGATGCCAAGAATCAGGAGTATA
AGTACTCAGTTGAAGAAGTAAATGTTCCAGACGGCTACAAGGTGTCGTAT
TTAGGAAATGATATATTTAACACCAGAGAAACAGAATTTGTGTTTGAACA
GAATAACTTTAACCTTGAATTTGGAAATGCTGAAATAAAAGGTCAATCTG
GGTCAAAAATCATTGATGAAGACACGCTAACGTCTTTCAAAGGTAAGAAA
ATTTGGAAAAATGATACGGCAGAAAATCGTCCCCAAGCCATTCAAGTGCA
GCTTTATGCTGATGGAGTGGCTGTGGAAGGTCAAACCAAATTTATTTCTG
GCTCAGGTAATGAGTGGTCATTTGAGTTTAAAAACTTGAAGAAGTATAAT
GGAACAGGTAATGACATCATTTACTCAGTTAAAGAAGTAACTGTTCCAAC
AGGTTATGATGTGACTTACTCAGCTAATGATATTATTAATACCAAACGTG
AGGTTATTACACAACAAGGACCGAAACTAGAGATTGAAGAAACGCTTCCG
CTAGAATCAGGTGCTTCAGGCGGTACCACTACTGTCGAAGACTCACGCCC
AGTTGATACCTTATCAGGTTTATCAAGTGAGCAAGGTCAGTCCGGTGATA
TGACAATTGAAGAAGATAGTGCTACCCATATTAAATTCTCAAAACGTGAT
ATTGACGGCAAAGAGTTAGCTGGTGCAACTATGGAGTTGCGTGATTCATC
TGGTAAAACTATTAGTACATGGATTTCAGATGGACAAGTGAAAGATTTCT
ACCTGATGCCAGGAAAATATACATTTGTCGAAACCGCAGCACCAGACGGT
TATGAGATAGCAACTGCTATTACCTTTACAGTTAATGAGCAAGGTCAGGT
TACTGTAAATGGCAAAGCAACTAAAGGTGACACTCATATTGTCATGGTTG
ATGCTTACAAGCCAACTAAGGGTTCAGGTCAGGTTATTGATATTGAAGAA
AAGCTTCCAGACGAGCAAGGTCATTCTGGTTCAACTACTGAAATAGAAGA
CAGTAAATCTTCAGACCTTATCATTGGCGGTCAAGGTGAAGTTGTTGACA
CAACAGAAGACACACAAAGTGGTATGACGGGCCATTCTGGCTCAACTACT
GAAATAGAAGATAGCAAGTCTTCAGACGTTATCATTGGTGGTCAGGGGCA
GGTTGTCGAGACAACAGAGGATACCCAAACTGGCATGTACGGGGATTCTG
GTTGTAAAACGGAAGTCGAAGATACTAAACTAGTACAATCCTTCCACTTT
GATAACAAGGAACCAGAAAGTAACTCTGAGATTCCTAAAAAAGATAAGCC
AAAGAGTAATACTAGTTTACCAGCAACTGGTGAGAAGCAACATAATATGT
TCTTTTGGATGGTTACTTCTTGCTCACTTATTAGTAGTGTTTTTGTAATA
TCACTAAAATCCAAAAAACGCCTATCATCATGTTAA

SEQ ID NO: 121

MTQKNSYKLSFLLSLTGFILGLLLVFIGLSGVSVGHAETRNGANKQGSFE
IKKVDQNNKPLPGATFSLTSKDGKGTSVQTFTSNDKGIVDAQNLQPGTYT
LKEETAPDGYDKTSRTWTVTVYENGYTKLVENPYNGEIISKAGSKDVSSS
LQLENPKMSVVSKYGKTEVSSGAADFYRNHAAYFKMSFELKQKDKSETIN
PGDTFVLQLDRRLNPKGISQDIPKIIYDSANSPLAIGKYHAENHQLIYTF
TDYIAGLDKVQLSAELSLFLENKEVLENTSISNFKSTIGGQEITYKGTVN
VLYGNESTKESNYITNGLSNVGGSIESYNTETGEFVWYVYVNPNRTNIPY
ATMNLWGFGRARSNTSDLENDANTSSAELGEIQVYEVPEGEKLPSSYGVD
VTKLTLRTDITAGLGNGFQMTKRQRIDFGNNIQNKAFIIKVTGKTDQSGK
PLVVQSNLASFRGASEYAAFTPVGGNVYFQNEIALSPSKGSGSGKSEFTK
PSITVANLKRVAQLRFKKMSTDNVPLPEAAFELRSSNGNSQKLEASSNTQ
GEVHFKDLTSGTYDLYETKAPKGYQQVTEKLATVTVDTTKPAEEMVTWGS
PHSSVKVEANKEVTIVNHKETLTFSGKKIWENDRPDQRPAKIQVQLLQNG
QKMPNQIQEVTKDNDWSYHFKDLPKYDAKNQEYKYSVEEVNVPDGYKVSY
LGNDIFNTRETEFVFEQNNFNLEFGNAEIKGQSGSKIIDEDTLTSFKGKK
IWKNDTAENRPQAIQVQLYADGVAVEGQTKFISGSGNEWSFEFKNLKKYN
GTGNDIIYSVKEVTVPTGYDVTYSANDIINTKREVITQQGPKLEIEETLP
LESGASGGTTTVEDSRPVDTLSGLSSEQGQSGDMTIEEDSATHIKFSKRD
IDGKELAGATMELRDSSGKTISTWISDGQVKDFYLMPGKYTFVETAAPDG
YEIATAITFTVNEQGQVTVNGKATKGDTHIVMVDAYKPTKGSGQVIDIEE
KLPDEQGHSGSTTEIEDSKSSDLIIGGQGEVVDTTEDTQSGMTGHSGSTT
EIEDSKSSDVIIGGQGQVVETTEDTQTGMYGDSGCKTEVEDTKLVQSFHF
DNKEPESNSEIPKKDKPKSNTSLPATGEKQHNMFFWMVTSCSLISSVFVI
SLKSKKRLSSC

19224141 contains an amino acid motif indicative of a cell wall anchor: SEQ ID NO:181 LPATG (shown in italics in SEQ ID NO:121, above). In some recombinant host cell systems, it may be preferable to remove this motif to facilitate secretion of a recombinant 19224141 protein from the host cell. Alternatively, in other recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

Two pilin motifs, discussed above, containing conserved lysine (K) residues have also been identified in 19224141. The pilin motif sequences are underlined in SEQ ID NO:121, below. Conserved lysine (K) residues are also marked in bold, at amino acid residues 157 and 163 and at amino acid residues 216, 224, and 238. The pilin sequence, in particular the conserved lysine residues, are thought to be important for the formation of oligomeric, pilus-like structures. Preferred fragments of 19224141 include at least one conserved lysine residue. Preferably, fragments include at least one pilin sequence.

SEQ ID NO: 121

MTQKNSYKLSFLLSLTGFILGLLLVFIGLSGVSVGHAETRNGANKQGSFE
IKKVDQNNKPLPGATFSLTSKDGKGTSVQTFTSNDKGIVDAQNLQPGTYT
LKEETAPDGYDKTSRTWTVTVYENGYTKLVENPYNGEIISKAGSKDVSSS
LQLENPKMSVVSKYGKTEVSSGAADFYRNHAAYFKMSFELKQKDKSETIN
PGDTFVLQLDRRLNPKGISQDIPKIIYDSANSPLAIGKYHAENHQLIYTF
TDYIAGLDKVQLSAELSLFLENKEVLENTSISNFKSTIGGQEITYKGTVN
VLYGNESTKESNYITNGLSNVGGSIESYNTETGEFVWYVYVNPNRTNIPY
ATMNLWGFGRARSNTSDLENDANTSSAELGEIQVYEVPEGEKLPSSYGVD
VTKLTLRTDITAGLGNGFQMTKRQRIDFGNNIQNKAFIIKVTGKTDQSGK
PLVVQSNLASFRGASEYAAFTPVGGNVYFQNEIALSPSKGSGSGKSEFTK
PSITVANLKRVAQLRFKKMSTDNVPLPEAAFELRSSNGNSQKLEASSNTQ
GEVHFKDLTSGTYDLYETKAPKGYQQVTEKLATVTVDTTKPAEEMVTWGS
PHSSVKVEANKEVTIVNHKETLTFSGKKIWENDRPDQRPAKIQVQLLQNG
QKMPNQIQEVTKDNDWSYHFKDLPKYDAKNQEYKYSVEEVNVPDGYKVSY
LGNDIFNTRETEFVFEQNNFNLEFGNAEIKGQSGSKIIDEDTLTSFKGKK
IWKNDTAENRPQAIQVQLYADGVAVEGQTKFISGSGNEWSFEFKNLKKYN
GTGNDIIYSVKEVTVPTGYDVTYSANDIINTKREVITQQGPKLEIEETLP
LESGASGGTTTVEDSRPVDTLSGLSSEQGQSGDMTIEEDSATHIKFSKRD
IDGKELAGATMELRDSSGKTISTWISDGQVKDFYLMPGKYTFVETAAPDG
YEIATAITFTVNEQGQVTVNGKATKGDTHIVMVDAYKPTKGSGQVIDIEE
KLPDEQGHSGSTTEIEDSKSSDLIIGGQGEVVDTTEDTQSGMTGHSGSTT
EIEDSKSSDVIIGGQGQVVETTEDTQTGMYGDSGCKTEVEDTKLVQSFHF
DNKEPESNSEIPKKDKPKSNTSLPATGEKQHNMFFWMVTSCSLISSVFVI
SLKSKKRLSSC

Two E boxes containing conserved glutamic residues have been identified in 19224141. The E-box motifs are underlined in SEQ ID NO:121, below. The conserved glutamic acid (E) residues, at amino acid residues 567 and 944, are marked in bold. The E box motifs, in particular the conserved glutamic acid residues, are thought to be important for the formation of oligomeric pilus-like structures of 19224141. Preferred fragments of 19224141 include at least one conserved glutamic acid residue. Preferably, fragments include at least one E box motif.

SEQ ID NO: 121

MTQKNSYKLSFLLSLTGFILGLLLVFIGLSGVSVGHAETRNGANKQGSFE
IKKVDQNNKPLPGATFSLTSKDGKGTSVQTFTSNDKGIVDAQNLQPGTYT
LKEETAPDGYDKTSRTWTVTVYENGYTKLVENPYNGEIISKAGSKDVSSS
LQLENPKMSVVSKYGKTEVSSGAADFYRNHAAYFKMSFELKQKDKSETIN
PGDTFVLQLDRRLNPKGISQDIPKIIYDSANSPLAIGKYHAENHQLIYTF
TDYIAGLDKVQLSAELSLFLENKEVLENTSISNFKSTIGGQEITYKGTVN
VLYGNESTKESNYITNGLSNVGGSIESYNTETGEFVWYVYVNPNRTNIPY
ATMNLWGFGRARSNTSDLENDANTSSAELGEIQVYEVPEGEKLPSSYGVD
VTKLTLRTDITAGLGNGFQMTKRQRIDFGNNIQNKAFIIKVTGKTDQSGK
PLVVQSNLASFRGASEYAAFTPVGGNVYFQNEIALSPSKGSGSGKSEFTK
PSITVANLKRVAQLRFKKMSTDNVPLPEAAFELRSSNGNSQKLEASSNTQ
GEVHFKDLTSGTYDLYETKAPKGYQQVTEKLATVTVDTTKPAEEMVTWGS
PHSSVKVEANKEVTIVNHKETLTFSGKKIWENDRPDQRPAKIQVQLLQNG
QKMPNQIQEVTKDNDWSYHFKDLPKYDAKNQEYKYSVEEVNVPDGYKVSY
LGNDIFNTRETEFVFEQNNFNLEFGNAEIKGQSGSKIIDEDTLTSFKGKK
IWKNDTAENRPQAIQVQLYADGVAVEGQTKFISGSGNEWSFEFKNLKKYN
GTGNDIIYSVKEVTVPTGYDVTYSANDIINTKREVITQQGPKLEIEETLP
LESGASGGTTTVEDSRPVDTLSGLSSEQGQSGDMTIEEDSATHIKFSKRD
IDGKELAGATMELRDSSGKTISTWISDGQVKDFYLMPGKYTFVETAAPDG
YEIATAITFTVNEQGQVTVNGKATKGDTHIVMVDAYKPTKGSGQVIDIEE
KLPDEQGHSGSTTEIEDSKSSDLIIGGQGEVVDTTEDTQSGMTGHSGSTT
EIEDSKSSDVIIGGQGQVVETTEDTQTGMYGDSGCKTEVEDTKLVQSFHF
DNKEPESNSEIPKKDKPKSNTSLPATGEKQHNMFFWMVTSCSLISSVFVI
SLKSKKRLSSC

As discussed above, applicants have also determined the nucleotide and encoded amino acid sequence of fimbrial structural subunits in several other GAS AI-4 strains of bacteria. Examples of sequences of these fimbrial structural subunits are set forth below.

M12 strain isolate 20010296 is a GAS AI-4 strain of bacteria. 20010296_fimbrial is thought to be a fimbrial structural subunit of M12 strain isolate 20010296. An example of a nucleotide sequence encoding the 20010296_fimbrial protein (SEQ ID NO:257) and a 20010296_fimbrial protein amino acid sequence (SEQ ID NO:258) are set forth below.

SEQ ID NO: 257

AGCAGTGGTCAATTAACAATAAAAAAATCAATTACAAATTTTAATGATGA
TACACTTTTGATGCCTAAGACAGACTATACTTTTAGCGTTAATCCGGATA
GTGCGGCTACAGGTACTGAAAGTAATTTACCAATTAAACCAGGTATTGCT
GTTAACAATCAAGATATTAAGGTTTCTTATTCTAATACTGATAAGACATC
AGGTAAAGAAAAACAAGTTGTTGTTGACTTTATGAAAGTTACTTTTCCTA
GCGTTGGTATTTACCGTTATGTTGTTACCGAGAATAAAGGGACAGCAGAA
GGAGTTACATATGATGATACAAAATGGTTAGTTGACGTCTATGTTGGTAA
TAATGAAAAGGGAGGTCTTGAACCAAAGTATATTGTATCTAAAAAAGGAG
ATTCTGCTACTAAAGAACCAATCCAGTTTAATAATTCATTCGAAACAACG
TCATTAAAAATTGAAAAGGAAGTTACTGGTAATACAGGAGATCATAAAAA
AGCATTTAACTTTACATTAACATTGCAACCAAATGAATACTATGAGGCAA
GTTCGGTTGTGAAAATTGAAGAGAACGGACAAACGAAAGATGTGAAAATT
GGGGAGGCATATAAGTTTACTTTGAACGATAGTCAGAGTGTGATATTGTC
TAAATTACCAGTTGGTATTAATTATAAAGTTGAAGAAGCAGAAGCTAATC
AAGGTGGATATACTACAACAGCAACTTTAAAAGATGGAGAAAAGTTATCT
ACTTATAACTTAGGTCAGGAACATAAAACAGACAAGACTGCTGATGAAAT
CGT

SEQ ID NO: 258

SSGQLTIKKSITNFNDDTLLMPKTDYTFSVNPDSAATGTESNLPIKPGIA
VNNQDIKVSYSNTDKTSGKEKQVVVDFMKVTFPSVGIYRYVVTENKGTAE
GVTYDDTKWLVDVYVGNNEKGGLEPKYIVSKKGDSATKEPIQFNNSFETT
SLKIEKEVTGNTGDHKKAFNFTLTLQPNEYYEASSVVKIEENGQTKDVKI
GEAYKFTLNDSQSVILSKLPVGINYKVEEAEANQGGYTTTATLKDGEKLS
TYNLGQEHKTDKTADEIV

M12 strain isolate 20020069 is a GAS AI-4 strain of bacteria. 20020069_fimbrial is thought to be a fimbrial structural subunit of M12 strain isolate 20020069. An example of a nucleotide sequence encoding the 20020069_{—} fimbrial protein (SEQ ID NO:259) and a 20020069_fimbrial protein amino acid sequence (SEQ ID NO:260) are set forth below.

SEQ ID NO: 259

AGCAGTGGTCAATTAACAATAAAAAAATCAATTACAAATTTTAATGATGA
TACACTTTTGATGCCTAAGACAGACTATACTTTTAGCGTTAATCCGGATA
GTGCGGCTACAGGTACTGAAAGTAATTTACCAATTAAACCAGGTATTGCT
GTTAACAATCAAGATATTAAGGTTTCTTATTCTAATACTGATAAGACATC
AGGTAAAGAAAAACAAGTTGTTGTTGACTTTATGAAAGTTACTTTTCCTA
GCGTTGGTATTTACCGTTATGTTGTTACCGAGAATAAAGGGACAGCAGAA
GGAGTTACATATGATGATACAAAATGGTTAGTTGACGTCTATGTTGGTAA
TAATGAAAAGGGAGGTCTTGAACCAAAGTATATTGTATCTAAAAAAGGAG
ATTCTGCTACTAAAGAACCAATCCAGTTTAATAATTCATTCGAAACAACG
TCATTAAAAATTGAAAAGGAAGTTACTGGTAATACAGGAGATCATAAAAA
AGCATTTAACTTTACATTAACATTGCAACCAAATGAATACTATGAGGCAA
GTTCGGTTGTGAAAATTGAAGAGAACGGACAAACGAAAGATGTGAAAATT
GGGGAGGCATATAAGTTTACTTTGAACGATAGTCAGAGTGTGATATTGTC
TAAATTACCAGTTGGTATTAATTATAAAGTTGAAGAAGCAGAAGCTAATC
AAGGTGGATATACTACAACAGCAACTTTAAAAGATGGAGAAAAGTTATCT
ACTTATAACTTAGGTCAGGAACATAAAACAGACAAGACTGCTGATGAAAT
CGT

SEQ ID NO: 260

SSGQLTIKKSITNFNDDTLLMPKTDYTFSVNPDSAATGTESNLPIKPGIA
VNNQDIKVSYSNTDKTSGKEKQVVVDFMKVTFPSVGIYRYVVTENKGTAE
GVTYDDTKWLVDVYVGNNEKGGLEPKYIVSKKGDSATKEPIQFNNSFETT
SLKIEKEVTGNTGDHKKAFNFTLTLQPNEYYEASSVVKIEENGQTKDVKI
GEAYKFTLNDSQSVILSKLPVGINYKVEEAEANQGGYTTTATLKDGEKLS
TYNLGQEHKTDKTADEIV

M12 strain isolate CDC SS 635 is a GAS AI-4 strain of bacteria. CDC SS 635_fimbrial is thought to be a fimbrial structural subunit of M12 strain isolate CDC SS 635. An example of a nucleotide sequence encoding the CDC SS 635_fimbrial protein (SEQ ID NO:261) and a CDC SS 635_fimbrial protein amino acid sequence (SEQ ID NO:262) are set forth below.

SEQ ID NO: 261

GAGACGGCAGGGGTTGTTAGCAGTGGTCAATTAACAATAAAAAAATCAAT
TACAAATTTTAATGATGATACACTTTTGATGCCTAAGACAGACTATACTT
TTAGCGTTAATCCGGATAGTGCGGCTACAGGTACTGAAAGTAATTTACCA
ATTAAACCAGGTATTGCTGTTAACAATCAAGATATTAAGGTTTCTTATTC
TAATACTGATAAGACATCAGGTAAAGAAAAACAAGTTGTTGTTGACTTTA
TGAAAGTTACTTTTCCTAGCGTTGGTATTTACCGTTATGTTGTTACCGAG
AATAAAGGGACAGCAGAAGGAGTTACATATGATGATACAAAATGGTTAGT
TGACGTCTATGTTGGTAATAATGAAAAGGGAGGTCTTGAACCAAAGTATA
TTGTATCTAAAAAAGGAGATTCTGCTACTAAAGAACCAATCCAGTTTAAT
AATTCATTCGAAACAACGTCATTAAAAATTGAAAAGGAAGTTACTGGTAA
TACAGGAGATCATAAAAAAGCATTTAACTTTACATTAACATTGCAACCAA
ATGAATACTATGAGGCAAGTTCGGTTGTGAAAATTGAAGAGAACGGACAA
ACGAAAGATGTGAAAATTGGGGAGGCATATAAGTTTACTTTGAACGATAG
TCAGAGTGTGATATTGTCTAAATTACCAGTTGGTATTAATTATAAAGTTG
AAGAAGCAGAAGCTAATCAAGGTGGATATACTACAACAGCAACTTTAAAA
GATGGAGAAAAGTTATCTACTTATAACTTAGGTCAGGAACATAAAACAGA
CAAGACTGCTGATGAAATCGTTGTCACAAATAACCGTGACACT

SEQ ID NO: 262

ETAGVVSSGQLTIKKSITNFNDDTLLMPKTDYTFSVNPDSAATGTESNLP
IKPGIAVNNQDIKVSYSNTDKTSGKEKQVVVDFMKVTFPSVGIYRYVVTE
NKGTAEGVTYDDTKWLVDVYVGNNEKGGLEPKYIVSKKGDSATKEPIQFN
NSFETTSLKIEKEVTGNTGDHKKAFNFTLTLQPNEYYEASSVVKIEENGQ
TKDVKIGEAYKFTLNDSQSVILSKLPVGINYKVEEAEANQGGYTTTATLK
DGEKLSTYNLGQEHKTDKTADEIVVTNNRDT

M5 strain isolate ISS 4883 is a GAS AI-4 strain of bacteria. ISS4883_fimbrial is thought to be a fimbrial structural subunit of M5 strain isolate ISS 4883. An example of a nucleotide sequence encoding the ISS4883_fimbrial protein (SEQ ID NO:265) and an ISS4883_fimbrial protein amino acid sequence (SEQ ID NO:266) are set forth below.

SEQ ID NO: 265

GAGACGGCAGGGGTTGTAACAGGAAAATCACTACAAGTTACAAAGACAAT
GACTTATGATGATGAAGAGGTGTTAATGCCCGAAACCGCCTTTACTTTTA
CTATAGAGCCTGATATGACTGCAAGTGGAAAAGAAGGCGACCTAGATATT
AAAAATGGAATTGTAGAAGGCTTAGACAAACAAGTAACAGTAAAATATAA
GAATACAGATAAAACATCTCAAAAAACTAAAATAGCACAATTTGATTTTT
CTAAGGTTAAATTTCCAGCTATAGGTGTTTACCGCTATATGGTTTCAGAG
AAAAACGATAAAAAAGACGGAATTAGGTACGATGATAAAAAGTGGACTGT
AGATGTTTATGTTGGGAATAAGGCCAATAACGAAGAAGGTTTCGAAGTTC
TATATATTGTATCAAAAGAAGGTACTTCTAGTACTAAAAAACCAATTGAA
TTTACAAACTCTATTAAAACTACTTCCTTAAAAATTGAAAAACAAATAAC
TGGCAATGCAGGAGATCGTAAAAAATCATTCAACTTCACATTAACATTAC
AACCAAGTGAATATTATAAAACCGGATCAGTTGTGAAAATCGAACAGGAT
GGAAGTAAAAAAGATGTGACGATAGGAACGCCTTACAAATTTACTTTGGG
ACACGGTAAGAGTGTCATGTTATCGAAATTACCAATTGGTATCAATTACT
ATCTTAGTGAAGACGAAGCGAATAAAGACGGTTACACTACAACGGCAACA
TTAAAAGAACAAGGCAAAGAAAAGAGTTCCGATTTCACTTTGAGTACTCA
AAACCAGAAAACAGACGAATCTGCTGACGAAATCGTTGTCACAAATAAGC
GTGACACTCTCGAG

SEQ ID NO: 266

ETAGVVTGKSLQVTKTMTYDDEEVLMPETAFTFTIEPDMTASGKEGDLDI
KNGIVEGLDKQVTVKYKNTDKTSQKTKIAQFDFSKVKFPAIGVYRYMVSE
KNDKKDGIRYDDKKWTVDVYVGNKANNEEGFEVLYIVSKEGTSSTKKPIE
FTNSIKTTSLKIEKQITGNAGDRKKSFNFTLTLQPSEYYKTGSVVKIEQD
GSKKDVTIGTPYKFTLGHGKSVMLSKLPIGINYYLSEDEANKDGYTTTAT
LKEQGKEKSSDFTLSTQNQKTDESADEIVVTNKRDTLE

M50 strain isolate ISS4538 is a GAS AI-4 strain of bacteria. ISS4538_fimbrial is thought to be a fimbrial structural subunit of M50 strain ISS 4538. An example of a nucleotide sequence encoding the ISS4538_fimbrial protein (SEQ ID NO:255) and an ISS4538_fimbrial protein amino acid sequence (SEQ ID NO:256) are set forth below.

SEQ ID NO: 255

ATGAAAAAAAATAAATTATTACTTGCTACTGCAATCTTAGCAACTGCTTT
AGGAACAGCTTCTTTAAATCAAAACGTAAAAGCTGAGACGGCAGGGGTTG
TTAGCAGTGGTCAATTAACAATAAAAAAATCAATTACAAATTTTAATGAT
GATACACTTTTGATGCCTAAGACAGACTATACTTTTAGCGTTAATCCGGA
TAGTGCGGCTACAGGTACTGAAAGTAATTTACCAATTAAACCAGGTATTG
CTGTTAACAATCAAGATATTAAGGTTTCTTATTCTAATACTGATAAGACA
TCAGGTAAAGAAAAACAAGTTGTTGTTGACTTTATGAAAGTTACTTTTCC
TAGCGTTGGTATTTACCGTTATGTTGTTACCGAGAATAAAGGGACAGCAG
AAGGAGTTACATATGATGATACAAAATGGTTAGTTGACGTCTATGTTGGT
AATAATGAAAAGGGAGGTCTTGAACCAAAGTATATTGTATCTAAAAAAGG
AGATTCTGCTACTAAAGAACCAATCCAGTTTAATAATTCATTCGAAACAA
CGTCATTAAAAATTGAAAAGAAAGTTACTGGTAATACAGGAGATCATAAA
AAAGCATTTAACTTTACATTAACATTGCAACCAAATGAATACTATGAGGC
AAGTTCGGTTGTGAAAATTGAAGAGAACGGACAAACGAAAGATGTGAAAA
TTGGGGAGGCATATAAGTTTACTTTGAACGATAGTCAGAGTGTGATATTG
TCTAAATTACCAGTTGGTATTAATTATAAAGTTGAAGAAGCAGAAGCTAA
TCAAGGTGGATATACTACAACAGCAACTTTAAAAGATGGAGAAAAGTTAT
CTACTTATAACTTAGGTCAGGAACATAAAACAGACAAGACTGCTGATGAA
ATCGTTGTCACAAATAANCGNGACACTCNAGTTCCAACNGGTGTNGTAGG
CACCCCNCCTCCATTCNCAGTTCTTANCATTGNGGCTANTGGTGGNGTNA
TNTATNTTACAAAACGNAAAAAAGNATAA

SEQ ID NO: 256

MKKNKLLLATAILATALGTASLNQNVKAETAGVVSSGQLTIKKSITNFND
DTLLMPKTDYTFSVNPDSAATGTESNLPIKPGIAVNNQDIKVSYSNTDKT
SGKEKQVVVDFMKVTFPSVGIYRYVVTENKGTAEGVTYDDTKWLVDVYVG
NNEKGGLEPKYIVSKKGDSATKEPIQFNNSFETTSLKIEKKVTGNTGDHK
KAFNFTLTLQPNEYYEASSVVKIEENGQTKDVKIGEAYKFTLNDSQSVIL
SKLPVGINYKVEEAEANQGGYTTTATLKDGEKLSTYNLGQEHKTDKTADE
IVVTNXRDTXVPTGVVGTPPPFXVLXIXAXGGVXYXTKRKKX

Examples of GAS AI-5 sequences from M2 strain isolate 10270 are set forth below.

MGAS10270_Spy0107 is a 33 kDa chaperonin which flanks GAS AI-5. An example of an amino acid sequence for MGAS10270_Spy0107 is shown below as SEQ ID NO:296.

SEQ ID NO: 296

MDKIIKSIAQSGAFRAYVLDSTETVALAQEKHNTLSSSTVALGRTLIANQ
ILAANQKGDSKITVKVIGDSSFGHIISVADTKGHVKGYIQNTGVDIKKTA
TGEVLVGPFMGNGHFVTIIDYGTGNPYTSTTPLITGEIGEDFAYYLTESE
QTPSAIGLNVLLDENDKVKVAGGFMVQVLPGASEEEIARYEKRLQEMPAI
SYLLASKNHVDALLEAIYGDEPYKRLSEEPLSFQCDCSRERFEAALMTLP
KADLQAMIDEDKGAEIVCQFCGTKYQFNESDLEAIINDKA

MGAS10270_Spy108 is a transcriptional regulator (RofA). An example of an amino acid sequence for MGAS10270_Spy108 is shown below as SEQ ID NO:297.

SEQ ID NO: 297

MISIFSLDRIEIGEYTYQRLIWLSKCRKRGPLSLIEKYLESSIESKCQLV
VLFFKTSYLPITEVAEKTGLTFLQINHYCEELNAFFPGSLSMTIQKRMIS
CQFTHPFKETYLYQLYASSNILQLLAFLIKNGSHSRPLTDFARSHFLSNS
SAYRMREALIPLLRNFELKLSKNKIVGEEYRIRYLIALLYSKFGIKVYDL
TQQDKNIIHSFLSHSSTHLKTSPWLSESFSFYDILLALSWKRHQFSVTIP
QTRIFQQLKKLFVYDSLKKSSRDIIETYCQLNFSAGDLDYLYLIYITANN
0SFASLQWTPEHIRQCCQLFEENDTFRLLLNPIITLLPNLKEQKASLVKA
LMFFSKSFLFNLQHFIPETNLFVSPYYKGNQKLYTSLKLIVEEWMAKLPG
KRYLNHKHFHLFCHYVEQILRNIQPPLVVVFVASNFINAHLLTDSFPRYF
SDKSIDFHSYYLLQDNVYQIPDLKPDLVITHSQLIPFVHHELTKGIAVAE
ISFDESILSIQELMYQVKEEKFQADLTKQLT

MGAS10270_Spy109 is a hypothetical protein. An example of an amino acid sequence for MGAS10270_Spy109 is shown below as SEQ ID NO:298. It contains a motif indicative of a cell wall anchor, IpxTG (SEQ ID NO:133).

SEQ ID NO: 298

MKLSKKLLYSAVVLATVAGPTVSPVAQFATSGIVVRAEDTRVPSQTQPDK
TTVNIYKLQGADFSKQPEGIKNENGEPIDITKLKDTFGTAVTYLPGVKFK
YYKVKNYSTSDDVLKSIKTVEQADSKTDLLDVAGAKETEATDQSGKVSID
LPSNDKVKYLFVESSNQDTVNKVVGYTAVPFILHLPVSNSNGKGYYDEVN
VYPKNTTVNEPKVDKDVTKLGKDDDTYQIGDKITWFLKSTVPSNIKTLDK
FGFTDTLNKGLSFIGDKTQTVTKVQFGTTVLSPDTDYTVEILDSKLTVSL
TSAGIEKVSGLVASKQLITEAEKLYKAEDNTDEAAFLSVEVNAKLNADAV
MGSRIENDVELDYGHESDIYKSKVPTNEVPEVHTGGARFEKVDATNQTDK
LQDAEFGLYSNIEATETVKWTEELLKANEAAINAGKFKESNTTVGTPITF
KSASDGSFEIKGLRYGDDSTNTRSDGTVGTAEKTGKTTYYIKELVAPKGY
VVSQDIVQFDVTYSSYYKDPTKVTLGTEAGDAAPTSVKNNKRPSIPNTGG
IGTAIFVAIGAAVMAFAAKGMKRRTEEN

MGAS10270_Spy0110 is a hypothetical protein. An example of an amino acid sequence for MGAS10270_Spy0110 is shown below as SEQ ID NO:299. It contains a motif indicative of a cell wall anchor, IpxTG (SEQ ID NO:133).

SEQ ID NO: 299

MKQTLKLMVSFLVMLGTMFGISQTVLAQGTHQLTIVHLEARDIDRPYPQL
DIAPKEGTPTEGVLYQLYQLKSTEDGDLLAHWNSLTITELKKQAQQVFEA
TTNQQGKATFNQLPDGTYYGLAVKAGEKDRNVSAFLVDLSEDKVIYPKII
WSTGELDLLKVGVDGDTKKPLAGVVFELYEKDGMVPIRVKNGVHSQDIDA
AKRLETDSSGHIRISGLIPGDYVLKEIETRSGYQIDQSETAVTIEKSKTV
TVTIKNQKIPSPKVPPRGGFIPKTGEQQAMILVIIGGILIALALRLLSKH
RKNQDKH

MGAS10270_Spy0111 is a sortase. An example of an amino acid sequence for MGAS10270_Spy0111 is shown below as SEQ ID NO:300.

SEQ ID NO: 300

MRKRSKTSLATNIRIWIFRLIFLAGFLVLAFPIVSQITYYQASHANINAF
KKAVAKIDQSEINRRLELAYAYNASIAGARKTGEHPVLKDPYSAEQKQAG
VIEYARMLEVKEQIGHVIIPRINQDIPIYAGSAEENLQRGVGHLEGTSLP
VGGESTHAVLTAHRGLPTAKLFTNLDKVTVGDRFYIEHIGGKIAYQVDQI
KVISPDQLEDLYVIQGEDHITLLTCTPYMINSHRLLVRGKRIPYVKKAVQ
KEAETFRQKQYLTYAIWVIVGLILLSFLIWFKKTKQKKRRENEKATSQIS
HNKSK

MGAS10270_Spy0112 is a sortase. An example of an amino acid sequence for MGAS10270_Spy0112 is shown below as SEQ ID NO:301.

SEQ ID NO: 301

MKKRLAKLATTIRNNKIRTLIFLLGSLILLFPIVSQVSYYLASHQNINRF
EREVSKMDTSAIDRRIALAKAYNETVSRNPLVIDPFTRKQKEGLREYARM
LEVHEQIGHVAVPSIGVDIPIYAGTAESVLQKGSGHLEGTSLPVGGQSTH
AVLTAHRGLPTARLFTDLNKVKKGQIFYVTNIKETLAYKVVSIKVVNPTA
LSTVKVVKGKDYMTLLTCTPYMINSHRLLIKGERIPYDSTKAEKCKEQAI
QGYHLSLVLKILLGLLIGLFIVIMMRRWMKHRQ

MGAS10270_Spy0113 is a collagen adhesion protein. An example of an amino acid sequence for MGAS10270_Spy0113 is shown below as SEQ ID NO:302. It contains a motif indicative of a cell wall anchor, FPxTG (SEQ ID NO:141).

SEQ ID NO: 302

MKKKQKLWRGLSVTLLILSQIPFGILVQGETQDNNPALGKVIVKKTGENA
NPLGKATFVLKNDHDKSEISHETVEGSGEAAFENIKPGNYTLTEKTAPIG
YKKTDKTWKVKVADNGATTIEDIDPDKVEKRKEALNGQYPESAIYEDTKE
SYPLVKVEDSKVGNQYKALNPINGEDGRREITEGWLSKKIKKVNELDKNK
YKIELTVEGKTIVETKELNQPLDVVLLLDNSNSMNNERAHNSQRALKAGE
AVEKLIDKITSNKDNRVALVTYASTIFDGTEATVSKGVADRNGKALNDSV
SWDYHKTTFTATTHNYSYLNLTNDANEVNILKSRIPKEAEHINGNRTLYQ
FGATFTQKALMKANEILETQSSNDRKKVIFHVTDGVPTMSYAINFNPYIS
TSYQNQFKSFLNKTPDRSGILQEDFIINGDDYQIVKGDGESFKLFSDRKV
PVPGGTTQAAYQVPQNQLSVMSNEGYAINRGYIYLYWRDYNWVYPFDPKT
KTVSATKQIKTHGEPTTLYFNGNIKPKGYDIFTVGIGVNGDPGATPLEAK
EFMQSISSKTENYTNVDDTNKIYDELNKYFKTIVEEKHSIVDGNVTDPMG
EMIEFQLKDGQSFTHDDYVLVGNDGSQLKNGVALGGPNSDGGILKDVTVT
YDKTSQTIKINHLNLGSGQKVVLTYDVRLKENYISNKFYNTNNRTTLSPK
SEKEPNTIRDFPIPKIRDVREFPVLTISNQKKMGEVEFIKVNKDKHSESL
LGAKFQLQIEKDFSGYKQFVPEGSDVTTKNDGKIYFKALQDGNYKLYEIS
SPDGYIEVKTKPVVTFTIQNGEVTNLKQDPNANKNQIGYFEEDGKHLITN
TPKRPPSVFPKTGGIGTIVYILVGCTLMIVATGSFRRNQQ

MGAS10270_Spy0114 is a hypothetical protein. An example of an amino acid sequence for MGAS10270_Spy0114 is shown below as SEQ ID NO:303.

SEQ ID NO: 303
MISSYLSLLSVIGLAKAYNASLSGTSSQATQSVLRDSYSEEQKRQGGLNTLGC

MGAS10270_Spy0115 is a sortase. An example of an amino acid sequence for MGAS10270_Spy0115 is shown below as SEQ ID NO:304.

SEQ ID NO: 304

MDKMRRGDRFYIEHLGGKLAYQVDSIKVITPDRLDDLYLVPGKDYVTLLT
CTPYMINSHRLLVRGKRIPYKEGLEHKDQQLGHRGQLITYGFVSLGLALV
LGLLWYRQRKK

MGAS10270_Spy0116 is a sortase. An example of an amino acid sequence for MGAS10270_Spy0116 is shown below as SEQ ID NO:305.

SEQ ID NO: 305

MKQRVKQLYQFIMTNKLRSFLFVLGILVVLFPVVSQISYYHASHQQINQF
EHKAAVIDRSAIERRISLAKAYNDAISRHPSLNDPFTSKEKAGLREYARM
LEVNEQIGHVAIPKIGVDLPIYAGTSAAILEKGSGHLEGTSLPIGGLSTH
AVLTAHRGLPTARLFTDLDKVKKGDYFYVTNLKETLAYQVDRIMVIEPSQ
LDAVSIEEGKDYVTLLTCTPYMINSHRLLVRGVRTPLTSRQAKKESLTAV
RPYQYYRWLVYLAIAVLVILVMIVAKYYQKTNKSS

MGAS10270_Spy0117 is a fibronectin binding protein. An example of an amino acid sequence for MGAS10270_Spy0117 is shown below as SEQ ID NO:306. It contains a motif indicative of a cell wall anchor, LPXTG (SEQ ID NO:122).

SEQ ID NO: 306

MSIVTNKTLHKVLVKLAAILAILFLVLPTGLTAVTVLAEQINTENLIPVE
TAAGKILKESDLDFEQAISTSEEALKDSSASWIQLTYNKVNQNLAKQDLF
LRLPQGMTIEGIGQDQKLDVKTISVPKDLDLDEAASTINNTNIITGFEKI
GEQDYKLSFAATSKTLNLVFKVKGTSLNEKRELQLSDKQNLNQTSFTKIL
GLGIAKSDDRTSANDTTTRPSIQPRAATARETTGNQDISANLTTSNAQLN
LEDKDKNNIYDVEAPDSFMFRATLSLLDSRYIVPGNYFELKLSDTIHYNM
LNPTDINFPYLTQDGKVIAVPELVQDPPVDGLLRATGKIVRYRFTDQVQG
LDSLTMTLNLGHSVNPNVVQNNDKYDFSYQIGGHIIKQSYDVQYGKPKEN
GNLNVHQRLTYTDSKENLKASSLIYVNPKQTPQYAGKQTLSILRNPKTYT
ISEQTVPNLLELNNQTSIKVYKLKDSKGVTDAVNLDKQYLEEVINPIITK
SDGKIEVSFNYTQGETSAYVVAVETSLQQEGSQPVFIAQNSNLTSASTRK
KVSETDATATVGSGGSASGDQTPKGTLYLNKVDTNHKHLANATFTLSGNT
TAGQFLYRTLITDTKEVSFSDLPPGSYLLKETTAPSGYQQITTPWTVTVD
DKGKVTVTGNEADTQKVPEEAEVVILWESTKWNYGNVQFYKDRFKELVDS
VGNQNAKYTLIRYSGDTSDSAQVINQSVSSAEFNKILNSETLTASTMTNR
KGMLKAYQLALQQFQASTNKRKYLLQLTNYPIYPGYREEKDFMSQSQTSF
DAMKSLGVVPYLLVERDPYANRNTYLDPKAEASSFGAFYPSDNIKVVWSN
NSSGLFTPPTNTNYTIQIQSIGSAIKQQIPESVLTVINRASGKFSINKID
EAKKGLAGATFTLSKRTTVAVNHQVQGAFTPVSKETTAGRTTLTFDNLKP
GVYDLKETKAPNAYVLDPKTYVVVVQNSGKTTIVDEANFKEADYPMADNT
SQFDYPTKDIANKPNKIVFTKIGDGGKSLSGAEFELRKGNEKVQTTTSGA
DGKVTFSKLLPGTYEVWETRVADSAYQLPQEAVATFEVKADGTFSEPMGR
LFRKNIAQNNRYEIRNELVNKKTGNQKIKVIKKDELTDLPLPNAKFQLEA
SDGTIYTKTTDVKGEIVFEKLPYGQYILTEIEAPSGYVLDPTPHKINLTE
VDSPETEVVAGRNATQPTVAPTARAALAAPVAEPVTGKNVSDQISIKELD
ITSSNEDTPRLVRPNHGENIVMRAGFTINPGSDIKAGDFFTLTLPNTIDP
FGVSAPENVDFRILGPLGTLALGTYDSSTHTITYKFTDYITRYTVSSFST
ISPFFIDRDRVKTNQDIDLFLKVGQVSSTSYHFTVDYNPYYGTADTNNPV
NVGSMITRLNQDTGDFVNYIYVNPAGQTLEQATLTFTGRGSTRIDANTRV
QLFEVTNPKLQMPPSWGIQDETLREVDPANYQLIKENGRFTINFYNDLLY
GRSYIVKVSGKSDKNDPDPIHTSAILTQRYFNDYPYYTPSGRYIPYGPYT
ESFTFTAEVVKKSGESNADGSVVVRLSNRKNYIDFMKSNSQGTPLEATFE
LRKKATNDTVTVGTPVISDKTTGKFYFEGLSPGDYEVWETKAPDGYTKPV
EAVATFKVTDEGEIVDKSLEDGRIINYKRPELPATGGPGIFVYLFIGSAM
CLVAIFWDHSFRFTK

MGAS10270_Spy0118 is a hypothetical protein which flanks GAS AI-5. An example of an amino acid sequence for MGAS10270_Spy0118 is shown below as SEQ ID NO:307.

SEQ ID NO: 307

MKRYNKYLFTSLLAASMLFSSYKSVHAHDNIDEKGKVHLYWQGNYYVDNY
VDYTKTLVDNNNSIEWTVTFNSAKEQWVYPDFSVFLPKGVKAPKEITYEH
HYWDGTVDSKTRRNTKWHYDWESQQTNFNQEFDKFPGYTGWSPSLDKFYK
LKNEGKFSHVLVDTYGRQSHTYFSHKMVWKFKTELEEDYKNKWDKLPFIA
GIKQNNPLAASFPSYKGEFGE

Examples of GAS AI-6 sequences from M4 strain isolate 10750 are set forth below.

MGAS10750_Spy0112 is a 33 kd chaperonin which flanks GAS AI-6. An example of an amino acid sequence for MGAS10750_Spy0112 is shown below as SEQ ID NO:308.

SEQ ID NO: 308

MDKIIKSIAQSGAFRAYVLDSTETVALAQEKHNTLSSSTVALGRTLIANQ
ILAANQKGDSKITVKVIGDSSFGHIISVADTKGHVKGYIQNTGVDIKKTA
TGEVLVGPFMGNGHFVTIIDYGTGNPYASTTPLITGEIGEDFAYYLTESE
QTPSAIGLNVLLDENDKVKVAGGFMVQVLPGASEEEIARYEKRLQEMPAI
SHLLASKNHVDALLEAIYGDEPYKRLSEEPLSFQCDCSRERFEAALMTLP
KADLQAMIDEDKGAEIVCQFCGTKYQFNESDLEALINDKA

MGAS10750_Spy0113 is a transcriptional regulator, rofA. An example of an amino acid sequence for MGAS10750_Spy0113is shown below as SEQ ID NO:309.

SEQ ID NO: 309

MISIFSLDRIEIGEYTYQRLIWLSKCRKRGPLYLIEKYLESSIESKCQLV
VLFFKTSYLPITEVAEKTGLTFLQLNHYCEELNAFFPGSLSMTIQKRMIS
CQFTHPFKETYLYQLYASSNVLQLLAFLIKNGSHSRPLTDFARSHFLSNS
SAYRMREALIPLLRNFELKLSKNKIVGEEYRIRYLIALLYSKFGIKVYDL
TQQDKNTIHSFLSHSSTHLKTSPWLSESFSFYDILLALSWKRHQFSVTIP
QTRIFQQLKKLFVYDSLKKSSHDIIETYCQLNFSAGDLDYLYLIYITANN
SFASLQWTPEHTRQCCQLFEENDTFRLLLNPIITLLPNLKEQKASLVKAL
MFFSKSFLFNLQHFIPETNLFVSPYYKGNQKLYTSLKLIVEEWMAKLPGK
RYLNHKHFHLFCHYVEQILRNIQPPLVVVFVASNFINAHLLTDSFPRYFS
DKSIDFHSYYLLQDNVYQIPDLKPDLVITHSQLIPFVQHELTKGIAVAEI
SFDESILSIQELMYQVKEEKFQADLTKQLT

MGAS10750_Spy0114 is a fibronectin binding protein. An example of an amino acid sequence for MGAS10750_Spy0114 is shown below as SEQ ID NO:310. It contains a motif indicative of a cell wall anchor, LPXTG (SEQ ID NO:122).

SEQ ID NO: 310

MVSSYMFVRGEKMNNKMFLNKEAGFLAHTKRKRRFAVTLVGVFFMLLAYA
GAIGFGQVAYAADEKTVPNFKSPDPDYPWYGYDAYGKGYPGYDISKYYHD
LRVNLNGSQVYQVYCFNIQKIFPYNVKSVTQKWFKKVEGNSDTFGLYAMN
PRVQGEELSQKLRSVMYNAYPKNANNIMDGLDTLNAIKVTQSAVWYYSDK
SEFEVDKQWESELNNHEIDQEQVTLMREALRKLISSNLEETVEKKLPENY
KLNIFNPQDTSIQHLLSAEFVPENPPRPGETPEYGPKTPELDGSPIPEDP
KHPDDNLEPTLPPVMLDGEEVPEVPSESLEPALPPLMPELDGQEVPEVPS
ESLEPALPPLMPELDGQEVPEKPSIDLPIEVPRYEFNNKDQSPLAGESGE
TEYITEVYGNQQNPVDIDKKLPNETGFSGNMVETEDTKEPEVLMGGQSES
VEFTKDTQTGMSGFSETATVVEDTRPKLVFHFDNNEPEVEENREKPTKNI
TPILPATGDIENVLAFLGILILSVLSIFSLLKNKQNKKV

MGAS10750_Spy0115 is a fibronectin binding protein. An example of an amino acid sequence for MGAS10750_Spy0115 is shown below as SEQ ID NO:311. It contains a motif indicative of a cell wall anchor, FPXTG (SEQ ID NO:141).

SEQ ID NO: 311

MYSRLKRELVIVINRKKKYKLIRLMVTLGLIFSQLAPPFGTLMALSGHSR
SKSPVTEVKADNVSTLKTGSFKLKKFDEDGKTPIKDVTFQLTSETNPSNY
KIEQITSGAGDASFANIPPGTYLLKEVAPPSGYQVMADYYRITVSPDGYT
QYTYVKVGTTTSSPTTSLPSTSGGGTGGTVFRTSKTSGVVTVTDYNFTTK
NKAQGNTDYTTLWATSGEFFDMSFKLKVNEGTQAGDSFTIKLSDYLSPNG
IREKFISAPPLMLDKKVVATGIYDESTNSYIYTFNDLINHKQNAEITVNY
TFSPEAKKVDRDWYVNTYNITNIIDGQKQDSGNFTIDYGQGQYMTGTLNS
GLRLRNNITYLNRTTGEVEYTIYLNNGASPRDKDFTVKNPVTGRHFLNLE
DKSASVAFTQKNITVYRVPLSQKTSKMPYSMSGETDGLESIPFDYSSKGI
TFTKESFHDNETNSNTAGLLIKIKAYITADNKRSADVSLSAGWTYTNLIR
SRSDAKASAFELGNTSSGVANNIEPTVTIRNYKIKKGSIVFTKQDVETKM
TLSGVAFRLEKKEENDWQIVEKYKDVQTGTEGKLILSDLDPGEYQLIETK
PLDGYLVSSGPVVTFTITDQGTEGTVKPSDKIIPNTKPGKQKIKVIKKDE
QSRVPLAGAKFQLESNKGVVLTGETDGKGEYTFTNLPFGDYILTEIAAPK
GYILDKTPRSIAIGDTVDKEPEPTVVEAATPRTVRSVSPSATVSDKDVSR
NILVKKVEFTTTNGQTPLQVKPNQGENLIARSEFELKKEIDIKKGDYFAV
KLSDNIDPFGVSTGETTTFNITGPYGTLAVGKYDSKSRSIIYTFTDYVEK
YEVSNFSTILPYFIDRYAVTRDADINISTSVGSQTNTARVRVLYTPYYGA
TDSYSPVNIGSMITKLDEKNGTFTNYIYINPMQQFIRNGKLTFQGGGSAI
IDNETQVTLFKVNNSTDMPPSWGITDSTLRVENDILVNKKQGEISVDFEN
ILEARNSFIMKVVGKIASSGTSVRTSATLSQKYDNTYGYYSTNGRWIPKG
PYSEYYRYDTTAILHSGDSNVDGAIRISVTNRKNSVAFTKTNGLEKPLEA
TFELRRLNSNKTFTSVKTTKSTKDTGKFSFEGMESGSYEVWETQSPEGYL
KPDKAVATFKVDKDGTIKDLTPDNGKIINYPNTAKIIFTKMLASEKEELS
PATKEKSATFSLWRLKEESLKETNTVSQAYDEQYYEPVMENNSVRTVTSD
SSGNVLFDKLSPGFYAIKEEKAPDGYVKQQGIVRIFQVDSSGKVIKYQYF
KDKSIAGKLTEITDLETEQLKQFNEIINKKFVFPMTGGQGIALLMIIGGT
MMGIAYFGHRRKQRLND

MGAS10750_Spy0116 is a cell wall surface anchor family protein. An example of an amino acid sequence for MGAS10750_Spy0116 is shown below as SEQ ID NO:312. It contains a motif indicative of a cell wall anchor, IPXTG (SEQ ID NO:133).

SEQ ID NO: 312

MKTKIFSRLAAVLLVLGSLLPTVVAVAEAESSHKTDVVIHKIKMTSLKGW
PKEKNPDGTYTGLGDKNYNGEKIDTITSYFGEGAEELDGVSFTYWSVDKE
KYKKLTKNPQNYDTVPKMKAFLQGTEKNKALENSSETIDGKTTGHTADKG
GVKVKDLADGYYWFVENSGSNIANGETLSSSAAVPFGLELPVYKADGSTI
TELHVYPKNTTTKPKIDKNFSKDEKDAALAGGANYDYYQKDKGYVSRIIG
SEVSYQIKTEIPAGAQYQTLRWEDTMTKGLTYKAGSLELTITTKGDGKAA
LNFEFQTDYKLTENQSGFVLKFTESGLEKVKKAVQATKDAKGQVTADGHP
MTVDISYKATVNSDAVVDQPDKNTVIFDYSNNPKEHKDPREKSTKPKDKQ
ITVEKTWASQSAPTGIAVTYYLYQKGDESGKDKVVDSVTLTTDYKHIFTN
LDDSKDYYVKESAIGYTPEYTEAKDGKISIKNTKDDKNPDPLKPTSPAIV
THGKKFVKTSQDDERLKGATFVVVDQKTNEKYLAIKADEKQSAEEKAYHD
TEQKYQDEVKKATTEKPNSQAIEKAKKARDDAFKKARTAYEWVSDKQKAV
KFTSNSKGQFEVTGLAAGTYYLEEVAAPTGFAKLQEKVEFTLGFNSYNGH
KDQSGQSLEGHTQYEKGTPDFGYGQRVINKKITIPQTGGIGTVIFTVVGL
AIMTVTGLMMIRRNKNDKSE

MGAS10750_Spy0117 is a cell wall surface anchor family protein. An example of an amino acid sequence for MGAS10750_Spy0117 is shown below as SEQ ID NO:313. It contains a motif indicative of a cell wall anchor, IPXTG (SEQ ID NO:133).

SEQ ID NO: 313

MGGTQLKIGRLIRLMLSICACLYFLTSPIFALQKTSSVTIHFENSDKDTQ
LALWQLPEGQTLPELETLFEKTDAELTRQYPQVSTVTVPKGETKLVLSNL
PVGAIYYVREAEERLGVRSLAPFILKVDTDDDQAVYTKKAKAQKRGSYPF
VKVSAQGGSLEGATFEVWKQTQKQLQPVIKGSSRYLLTSSKDGSFMARDL
PFGSYVLKEITAPKGYLLSKKTIPFEVTDYSEKQAPVKVVNQPKIPPRIE
IPYTGNAIMILVVLLGFALFTLGVYLVRRNG

MGAS10750_Spy0118 is a sortase. An example of an amino acid sequence for MGAS10750_Spy0118 is shown below as SEQ ID NO:314.

SEQ ID NO: 314

MKKQSKRHRLNQNIALIVVFLIGLTILLYPQISRIYYTIESNYQSKQFDR
EKSTLHQEDISQRIALAKAFNASLHDVDLKDPYSDDEKTKGPAEYARMLE
LHEQIGHVEIPKIRVDLPIYAGTSDEIISKGSGHLEGTSLPVGGENTHTV
LTSHSGLPSAKLFSDLAKLKKGDIFYIHNIKEILAYQIDQIKIIEPDNFS
DLLIVPGKDYATLLTCTPVGINTHRLIVRGHRVPYVSNEHKNNKQKASSG
YMCYLFVLLALLTLLFGYWFYRQKKKKSQKVKREEFHAKE

MGAS10750_Spy0119 is a sortase. An example of an amino acid sequence for MGAS10750_Spy0119 is shown below as SEQ ID NO:315.

SEQ ID NO: 315

MLKNKQSLSLRERLIQVMFPLLFLMGCLIVLYPLMSNYYYRVKQNQAVTS
FESAKVIVNKDDIKRRMALARAYNATLDPGRINDPYTDLEKKGVAEYARM
LELNEQIGYVEVPRFDINLPIYAGTSDDVLQKAAGHLEGTSLPIGGDSTH
TVITAHTGLPQAKLFTNIHKMKKGDLFFIHNIDKTLAYKVDQILVVEPDN
FTPVLVKNGFDYATLLTCTPYGINSHRLLVRGYRVPYQKAFEKADAQRPW
YTKVIFLVSFLLFVILVIILLIDWHRK

MGAS10750_Spy0120 is a sortase. An example of an amino acid sequence for MGAS10750_Spy0120 is shown below as SEQ ID NO:316.

SEQ ID NO: 316

MSKQKCVGYVLMILGLGLPLFFLTLMSLNQFQEQVAYQKFQTENRSWKNS
QKEWVNRHNQEQALADRATTDPFVDAQNQLKQSPFDDNIIGYIIIPKLRM
AQPIRVGASERHLEKGVAQVTGTSLPIGGLGTRSVIAGHRSWYDNERFLR
IAELSLGDQIVIDLGVYQLEYRVKSVEIIDAKDWRQLTAKKSQDLITLLT
CNPLYPPFNERLLVMQRECCLAFLIYQLKREIRWINLN

MGAS10750_Spy0121 is a hypothetical protein which flanks GAS AI-6. An example of an amino acid sequence for MGAS10750_Spy0121 is shown below as SEQ ID NO:317.

SEQ ID NO: 317

MKRCNKYLFTSLLAASMLFSSYKSVHAHDNIDEEGKVHLYWQGNYYVDNY
VDYTKKLVNNSIEWTVTFNSAKEQWVYPDFSVFLPKGVKAPKEITYEHHY
GNGTVDSKKRSNTQWHYDWEKQKTNFNQEFDKFPGYTGWSRSLDKFYKLK
NEGKFSHVLVDTYGRQSHTYFSHKMVWKFKTELEDNYKDKWNKLPFIAGI
KQNNPLAASFPSYKGEFGE

There may be an upper limit to the number of GAS proteins which will be in the compositions of the invention. Preferably, the number of GAS proteins in a composition of the invention is less than 20, less than 19, less than 18, less than 17, less than 16, less than 15, less than 14, less than 13, less than 12, less than 11, less than 10, less than 9, less than 8, less than 7, less than 6, less than 5, less than 4, or less than 3. Still more preferably, the number of GAS proteins in a composition of the invention is less than 6, less than 5, or less than 4. Still more preferably, the number of GAS proteins in a composition of the invention is 3.

The GAS proteins and polynucleotides used in the invention are preferably isolated, i.e., separate and discrete, from the whole organism with which the molecule is found in nature or, when the polynucleotide or polypeptide is not found in nature, is sufficiently free of other biological macromolecules so that the polynucleotide or polypeptide can be used for its intended purpose.

### Examples Other Gram Positive Bacterial Adhesin Island Sequences

The Gram positive bacteria AI polypeptides of the invention can, of course, be prepared by various means (e.g. recombinant expression, purification from a gram positive bacteria, chemical synthesis etc.) and in various forms (e.g. native, fusions, glycosylated, non-glycosylated etc.). They are preferably prepared in substantially pure form (i.e. substantially free from other streptococcal or host cell proteins) or substantially isolated form.

The Gram positive bacteria AI proteins of the invention may include polypeptide sequences having sequence identity to the identified Gram positive bacteria proteins. The degree of sequence identity may vary depending on the amino acid sequence (a) in question, but is preferably greater than 50% (e.g. 60%, 65%, 70%, 75%, 80%, 85%, 90%, 91%, 92%, 93%, 94%, 95%, 96%, 97%, 98%, 99%, 99.5%, or more). Polypeptides having sequence identity include homologs, orthologs, allelic variants and mutants of the identified Gram positive bacteria proteins. Typically, 50% identity or more between two proteins is considered to be an indication of functional equivalence. Identity between proteins is preferably determined by the Smith-Waterman homology search algorithm as implemented in the MPSRCH program (Oxford Molecular), using an affinity gap search with parameters gap open penalty=12 and gap extension penalty=1.

The Gram positive bacteria adhesin island polynucleotide sequences may include polynucleotide sequences having sequence identity to the identified Gram positive bacteria adhesin island polynucleotide sequences. The degree of sequence identity may vary depending on the polynucleotide sequence in question, but is preferably greater than 50% (e.g. 60%, 65%, 70%, 75%, 80%, 85%, 90%, 91%, 92%, 93%, 94%, 95%, 96%, 97%, 98%, 99%, 99.5% or more).

The Gram positive bacteria adhesin island polynucleotide sequences of the invention may include polynucleotide fragments of the identified adhesin island sequences. The length of the fragment may vary depending on the polynucleotide sequence of the specific adhesin island sequence, but the fragment is preferably at least 10 consecutive polynucleotides, (e.g. at least 10, 12, 14, 16, 18, 20, 25, 30, 35, 40, 50, 60, 70, 80, 90, 100, 150, 200 or more).

The Gram positive bacteria adhesin island amino acid sequences of the invention may include polypeptide fragments of the identified Gram positive bacteria proteins. The length of the fragment may vary depending on the amino acid sequence of the specific Gram positive bacteria antigen, but the fragment is preferably at least 7 consecutive amino acids, (e.g. 8, 10, 12, 14, 16, 18, 20, 25, 30, 35, 40, 50, 60, 70, 80, 90, 100, 150, 200 or more). Preferably the fragment comprises one or more epitopes from the sequence. The fragment may comprise at least one T-cell or, preferably, a B-cell epitope of the sequence. T- and B-cell epitopes can be identified empirically (e.g., using PEPSCAN [Geysen et al. (1984) PNAS USA 81:3998-4002; Carter (1994) Methods Mol. Biol. 36:207-223, or similar methods], or they can be predicted (e.g., using the Jameson-Wolf antigenic index [Jameson, B A et al. 1988, CABIOS 4(1): 1818-186], matrix-based approaches [Raddrizzani and Hammer (2000) Brief Bioinform. 1(2):179-189], TEPITOPE [De Lalla et al. (199) J. Immunol. 163:1725-1729], neural networks [Brusic et al. (1998) Bioinformatics 14(2):121-130], OptiMer & EpiMer [Meister et al. (1995) Vaccine 13(6):581-591; Roberts et al. (1996) AIDS Res. Hum. Retroviruses 12(7):593-610], ADEPT [Maksyutov & Zagrebelnaya (1993) Comput. Appi. Biosci. 9(3):291-297], Tsites [Feller & de la Cruz (1991) Nature 349(6311):720-721], hydrophilicity [Hopp (1993) Peptide Research 6:183-190], antigenic index [Welling et al. (1985) FEBS Lett. 188:215-218] or the methods disclosed in Davenport et al. (1995) Immunogenetics 42:392-297, etc. Other preferred fragments include (1) the N-terminal signal peptides of each identified Gram positive bacteria protein, (2) the identified Gram positive bacteria protein without their N-terminal signal peptides, (3) each identified Gram positive bacteria protein wherein up to 10 amino acid residues (e.g. 1, 2, 3, 4, 5, 6, 7, 8, 9, 10, 15, 20, 25 or more) are deleted from the N-terminus and/or the C-terminus e.g. the N-terminal amino acid residue may be deleted. Other fragments omit one or more domains of the protein (e.g. omission of a signal peptide, of a cytoplasmic domain, of a transmembrane domain, or of an extracellular domain), and (4) the polypeptides, but without their N-terminal amino acid residue.

As indicated in the above text, nucleic acids and polypeptides of the invention may include sequences that:
(a) are identical (i.e., 100% identical) to the sequences disclosed in the sequence listing;
(b) share sequence identity with the sequences disclosed in the sequence listing;
(c) have 1, 2, 3, 4, 5, 6, 7, 8, 9, or 10 single nucleotide or amino acid alterations (deletions, insertions, substitutions), which may be at separate locations or may be contiguous, as compared to the sequences of (a) or (b);
(d) when aligned with a particular sequence from the sequence listing using a pairwise alignment algorithm, a moving window of x monomers (amino acids or nucleotides) moving from start (N-terminus or 5′) to end (C-terminus or 3′), such that for an alignment that extends to p monomers (where p>x) there are p−x+1 such windows, each window has at least xy identical aligned monomers, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if xy is not an integer then it is rounded up to the nearest integer. The preferred pairwise alignment algorithm is the Needleman-Wunsch global alignment algorithm [Needlman &Wunsch (1970) J. Mol. Biol. 48, 443-453], using default parameters (e.g., with Gap opening penalty=10.0, and with Gap extension penalty=0.5, using the EBLOSUM62 scoring matrix). This algorithm is conveniently implemented in the needle tool in the EMBOSS package [Rice et al. (2000) Trends Genet. 16:276-277].

The nucleic acids and polypeptides of the invention may additionally have further sequences to the N-terminus/5′ and/or C-terminus/3′ of these sequences (a) to (d).

All of the Gram positive bacterial sequences referenced herein are publicly available through PubMed on GenBank.

Streptococcus pneumoniae Adhesin Island Sequences

As discussed above, a S. pneumoniae AI sequence is present in the TIGR4 S. pneumoniae genome. Examples of S. pneumoniae AI sequences are set forth below.

SrtD (Sp0468) is a sortase. An example of an amino acid sequence of SrtD is set forth in SEQ ID NO:80.

SEQ ID NO: 80

MSRTKLRALLGYLLMLVACLIPIYCFGQMVLQSLGQVKGHATFVKSMTTE
MYQEQQNHSLAYNQRLASQNRIVDPFLAEGYEVNYQVSDDPDAVYGYLSI
PSLEIMEPVYLGADYHHLGMGLAHVDGTPLPLDGTGIRSVIAGHPAEPSH
VFFRHLDQLKVGDALYYDNGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFLGILFVLWKLARLLRGK

SrtC (Sp0467) is a sortase. An example of an amino acid sequence of SrtC is set forth in SEQ ID NO:81.

SEQ ID NO: 81

MSRYYYRIESNEVIKEFDETVSQMDKAELEERWRLAQAFNATLKPSEILD
PFTEQEKKKGVSEYANMLKVHERIGYVEIPAIDQEIPMYVGTSEDILQKG
AGLLEGASLPVGGENTHTVITAHRGLPTAELFSQLDKMKKGDIFYLHVLD
QVLAYQVDQIVTVEPNDFEPVLIQHGEDYATLLTCTPYMINSHRLLVRGK
RIPYTAPIAERNRAVRERGQFWLWLLLGAMAVILLLLYRVYRNRRITVKG
LEKQLEGRHVKD

SrtB (SP0466) is a sortase. An example of an amino acid sequence of SrtB is set forth in SEQ ID NO:82.

SEQ ID NO: 82

MAVMAYPLVSRLYYRVESNQQIADFDKEKATLDEADIDERMKLAQAFNDS
LNNVVSGDPWSEEMKKKGRAEYARMLEIHERMGHVEIPVIDVDLPVYAGT
AEEVLQQGAGHLEGTSLPIGGNSTHAVITAHTGLPTAKMFTDLTKLKVGD
KFYVHNIKEVMAYQVDQVKVIEPTNFDDLLIVPGHDYVTLLTCTPYMINT
HRLLVRGHRIPYVAEVEEEFIAANKLSHLYRYLFYVAVGLIVILLWIIRR
LRKKKKQPEKALKALKAARKEVKVEDGQQ

Sp0465 is a hypothetical protein. An example of an amino acid sequence of Sp0465 is set forth in SEQ ID NO:83.

SEQ ID NO: 83

MFLPFLSASLYLQTHHFIAFPNRQSYLLRETRKSHFFLIHHPF

RrgC (SP0464) is a cell wall surface anchor family protein. RrgC contains a sortase substrate motif VPXTG (SEQ ID NO:137), shown in italics in SEQ ID NO:84.

SEQ ID NO: 84

MISRIFFVMALCFSLVWGAHAVQAQEDHTLVLQLENYQEVVSQLPSRDGH
RLQVWKLDDSYSYDDRVQIVRDLHSWDENKLSSFKKTSFEMTFLENQIEV
SHIPNGLYYVRSIIQTDAVSYPAEFLFEMTDQTVEPLVIVAKKTDTMTTK
VKLIKVDQDHNRLEGVGFKLVSVARDVSEKEVPLIGEYRYSSSGQVGRTL
YTDKNGEIFVTNLPLGNYRFKEVEPLAGYAVTTLDTDVQLVDHQLVTITV
VNQKLPRGNVDFMKVDGRTNTSLQGAMFKVMKEESGHYTPVLQNGKEVVV
TSGKDGRFRVEGLEYGTYYLWELQAPTGYVQLTSPVSFTIGKDTRKELVT
VVKNNKRPRIDVPDTGEETLYILMLVAILLFGSGYYLTKKPNN

RrgB (Sp0463) is a cell wall surface anchor protein. RrgB contains a sortase substrate motif IPXTG (SEQ ID NO:133), shown in italics in SEQ ID NO:85.

SEQ ID NO: 85

MKSINKFLTMLAALLLTASSLFSAATVFAAGTTTTSVTVHKLLATDGDMD
KIANELETGNYAGNKVGVLPANAKEIAGVMFVWTNTNNEIIDENGQTLGV
NIDPQTFKLSGAMPATAMKKLTEAEGAKFNTANLPAAKYKIYEIHSLSTY
VGEDGATLTGSKAVPIEIELPLNDVVDAHVYPKNTEAKPKIDKDFKGKAN
PDTPRVDKDTPVNHQVGDVVEYEIVTKIPALANYATANWSDRMTEGLAFN
KGTVKVTVDDVALEAGDYALTEVATGFDLKLTDAGLAKVNDQNAEKTVKI
TYSATLNDKAIVEVPESNDVTFNYGNNPDHGNTPKPNKPNENGDLTLTKT
WVDATGAPIPAGAEATFDLVNAQTGKVVQTVTLTTDKNTVTVNGLDKNTE
YKFVERSIKGYSADYQEITTAGEIAVKNWKDENPKPLDPTEPKVVTYGKK
FVKVNDKDNRLAGAEFVIANADNAGQYLARKADKVSQEEKQLVVTTKDAL
DRAVAAYNALTAQQQTQQEKEKVDKAQAAYNAAVIAANNAFEWVADKDNE
NVVKLVSDAQGRFEITGLLAGTYYLEETKQPAGYALLTSRQKFEVTATSY
SATGQGIEYTAGSGKDDATKVVNKKITIPQTGGIGTIIFAVAGAAIMGIA
VYAYVKNNKDEDQLA

RrgA (Sp0462) is a cell wall surface anchor protein. RrgA contains a sortase substrate motif YPXTG (SEQ ID NO:186), indicated in italics in SEQ ID NO:86.

SEQ ID NO: 86

MLNRETHMKKVRKIFQKAVAGLCCISQLTAFSSIVALAETPETSPAIGKV
VIKETGEGGALLGDAVFELKNNTDGTTVSQRTEAQTGEAIFSNIKPGTYT
LTEAQPPVGYKPSTKQWTVEVEKNGRTTVQGEQVENREEALSDQYPQTGT
YPDVQTPYQIIKVDGSEKNGQHKALNPNPYERVIPEGTLSKRIYQVNNLD
DNQYGIELTVSGKTVYEQKDKSVPLDVVILLDNSNSMSNIRNKNARRAER
AGEATRSLIDKITSDSENRVALVTYASTIFDGTEFTVEKGVADKNGKRLN
DSLFWNYDQTSFTTNTKDYSYLKLTNDKNDIVELKNKVPTEAEDHDGNRL
MYQFGATFTQKALMKADEILTQQARQNSQKVIFHITDGVPTMSYPINFNH
ATFAPSYQNQLNAFFSKSPNKDGILLSDFITQATSGEHTIVRGDGQSYQM
FTDKTVYEKGAPAAFPVKPEKYSEMKAAGYAVIGDPINGGYIWLNWRESI
LAYPFNSNTAKITNHGDPTRWYYNGNIAPDGYDVFTVGIGINGDPGTDEA
TATSFMQSISSKPENYTNVTDTTKILEQLNRYFHTIVTEKKSIENGTITD
PMGELIDLQLGTDGRFDPADYTLTANDGSRLENGQAVGGPQNDGGLLKNA
KVLYDTTEKRIRVTGLYLGTDEKVTLTYNVRLNDEFVSNKFYDTNGRTTL
HPKEVEQNTVRDFPIPKIRDVRKYPEITISKEKKLGDIEFIKVNKNDKKP
LRGAVFSLQKQHPDYPDIYGAIDQNGTYQNVRTGEDGKLTFKNLSDGKYR
LFENSEPAGYKPVQNKPIVAFQIVNGEVRDVTSIVPQDIPAGYEFTNDKH
YITNEPIPPKREYPRTGGIGMLPFYLIGCMMMGGVLLYTRKHP

RlrA (Sp0461) is a transcriptional regulator. An example of an amino acid sequence for RlrA is set forth in SEQ ID NO:87.

SEQ ID NO: 87

MLNKYIEKRITDKITILNILLDIRSIELDELSTLTSLQSKSLLSILQELQ
ETFEEELTFNLDTQQVQLIEHHSHQTNYYFHQLYNQSTILKILRFFLLQG
NQSFNEFTQKEYISIATGYRVRQKCGLLLRSVGLDLVKNQVVGPEYRIRF
LIALLQFHFGIEIYDLNDGSMDWVTHMIVQSNSQLSHELLEITPDEYVHF
SILVALTWKRREFPLEFPESKEFEKLKNLFMYPILMEHCQTYLEPHANMT
FTQEELDYIFLVYCSANSSFSKDKWNQEKKTHTIQLILQHTRGKHLLSKF
KNILGNDISNSLSFLTALTFLTRTFLFGLQNLVPYYNYYEHYGIESDKPL
YHISKAIVQEWMTEQKIEGVIDQHRLYLFSLYLTETIFSSLPAIPIFIIL
NNQADVNLIKSIILRNFTDKVASVTGYNILISPPPSEEHLTEPLIIITTK
EYLPYVKKQYPKGKHHFLTIALDLHVSQQRLIYQTIVDIRKEAFDKRVAM
IAKKAHYLL

As discussed above, a S. pneumoniae AI sequence is present in the S. pneumoniae strain 670 genome. Examples of S. pneumoniae AI sequences are set forth below.

Orf1_{—}670 is a transposase. An example of an amino acid sequence of orf1_{—}670 is set forth in SEQ ID NO:171.

SEQ ID NO: 171

MEHINHTTLLIGIKDKNITLNKAIQHDTHIEVFATLDYHPPKCKHCKGKQ
IKYDFQKPSKIPFIEIGGFPSLIHLKKRRFQCKSCRKVTVAETTLVQKNC
QISEMVRQKIAQLLLNREALTHIASKLAISTSTSTVYRKLKQFHFQEDYT
TLPEILSWDEFSYQKGKLAFIAQDFNTKKIMTILDNRRQTTIRNHFFKYS
KEARKKVKVVTVDMSGSYIPLIKKLFPNAKIVLDRFHIVQHMSRALNQTR
INIMKQFDDKSLEYRALKYYWKFILKDSRKLSLKPFYARTFRETLTPREC
LKKIFTLVPELKDYYDLYQLLLFHLQEKNTDQFWGLIQDTLPHLNRTFKT
TLSTFICYKNYITNAIELPYSNAKLEATNKLIKDIKRNAFGFRNFENFKK
RIFIALNIKKERTKFVLSRA

Orf2_{—}670 is a transcriptional regulator. An example of an amino acid sequence of Orf2_{—}670 is set forth in SEQ ID NO:172.

SEQ ID NO: 172

MLNKYIEKRITDKITILNILLDIRSIELDELSTLTSLQSKSLLSILQELQ
ETFEEELTFNLDTQQVQLIEHHSHQTNYYFHQLYNQSTILKILRFFLLQG
NQSFNEFTQKEYISIATGYRVRQKCGLLLRSVGLDLVKNQVVGPEYRIRF
LIALLQFHFGIEIYDLNDGSMDWVTHMIVQSNSQLSHELLEITPDEYVHF
SILVALTWKRREFPLEFPESKEFEKLKNLFMYPILMEHCQTYLEPHANMT
FTQEELDYIFLVYCSANSSFSKDKWNQEKKTHTIQLILQHTRGKHLLSKF
KNILGNDISNSLSFLTALTFLTRTFLFGLQNLVPYYNYYEHYGIESDKPL
YHISKAIVQEWMTEQKIEGVIDQHRLYLFSLYLTETIFSSLPAIPIFIIL
NNQADVNLIKSIILRNFTDKVASVTGYNILTSPPPSEEHLTEPLIIITTK
EYLPYVKKOYPKGKHHFLTIALDLHVSOORLIYQTIVDIRKEAFDKRVAM
IAKKAHYLL

Orf3_{—}670 is a cell wall surface anchor family proten. An example of an amino acid sequence of Orf3_{—}670 is set forth in SEQ ID NO:173.

SEQ ID NO: 173

MLNRETHMKKVRKIFQKAVAGLCCISQLTAFSSIVALAETPETSPAIGKV
VIKETGEGGALLGDAVFELKNNTDGTTVSQRTEAQTGEAIFSNIKPGTYT
LTEAQPPVGYKPSTKQWTVEVEKNGRTTVQGEQVENREEALSDQYPQTGT
YPDVQTPYQIIKVDGSEKNGQHKALNPNPYERVIPEGTLSKRIYQVNNLD
DNQYGIELTVSGKTTVETKEASTPLDVVILLDNSNSMSNIRHNHAHRAEK
AGEATRALVDKITSNPDNRVALVTYGSTIFDGSEATVEKGVADANGKILN
DSALWTFDRTTFTAKTYNYSFLNLTSDPTDIQTIKDRIPSDAEELNKDKL
MYQFGATFTQKALMTADDILTKQARPNSKKVIFHITDGVPTMSYPINFKY
TGTTQSYRTQLNNFKAKTPNSSGILLEDFVTWSADGEHKIVRGDGESYQM
FTKKPVTDQYGVHQILSITSMEQRAKLVSAGYRFYGTDLYLYWRDSILAY
PFNSSTDWITNHGDPTTWYYNGNMAQDGYDVFTVGVGVNGDPGTDEATAT
RFMQSISSSPDNYTNVADPSQILQELNRYFYTIVNEKKSIENGTITDPMG
ELIDFQLGADGRFDPADYTLTANDGSSLVNNVPTGGPQNDGGLLKNAKVF
YDTTEKRIRVTGLYLGTGEKVTLTYNVRLNDQFVSNKFYDTNGRTTLHPK
EVEKNTVRDFPIPKIRDVRKYPEITIPKEKKLGEIEFIKINKNDKKPLRD
AVFSLQKQHPDYPDIYGAIDQNGTYQNVRTGEDGKLTFKNLSDGKYRLFE
NSEPAGYKPVQNKPIVAFQIVNGEVRDVTSIVPQDIPAGYEFTNDKHYIT
NEPIPPKREYPRTGGIGMLPFYLIGCMMMGGVLLYTRKHP

Orf4_{—}670 is a cell wall surface anchor family protein. An example of an amino acid sequence of orf4_{—}670 is set forth in SEQ ID NO:174.

SEQ ID NO: 174

MKSINKFLTMLAALLLTASSLFSAATVFAADNVSTAPDAVTKTLTIHKLL
LSEDDLKTWDTNGPKGYDGTQSSLKDLTGVVAEEIPNVYFELQKYNLTDG
KEKENLKDDSKWTTVHGGLTTKDGLKIETSTLKGVYRIREDRTKTTYVGP
NGQVLTGSKAVPALVTLPLVNNNGTVIDAHVFPKNSYNKPVVDKRIADTL
NYNDQNGLSIGTKIPYVVNTTIPSNATFATSFWSDEMTEGLTYNEDVTIT
LNNVAMDQADYEVTKGNNGFNLKLTEAGLAKINGKDADQKIQITYSATLN
SLAVADIPESNDITYHYGNHQDHGNTPKPTKPNNGQITVTKTWDSQPAPE
GVKATVQLVNAKTGEKVGAPVELSENNWTYTWSGLDNSIEYKVEEEYNGY
SAEYTVESKGKLGVKNWKDNNPAPINPEEPRVKTYGKKFVKVDQKDTRLE
NAQFVVKKADSNKYIAFKSTAQQAADEKAAATAKQKLDAAVAAYTNAADK
QAAQALVDQAQQEYNVAYKEAKFGYVEVAGKDEAMVLTSNTDGQFQISGL
AAGTYKLEEIKAPEGFAKIDDVEFVVGAGSWNQGEFNYLKDVQKNDATKV
VNKKITIPQTGGIGTIIFAVAGAAIMGIAVYAYVKNNKDEDQLA

Orf5_{—}670 is a cell wall surface anchor family protein. An example of an amino acid sequence of orf5_{—}670 is set forth in SEQ ID NO:175.

SEQ ID NO: 175

MTMQKMQKMISRIFFVMALCFSLVWGAHAVQAQEDHTLVLQLENYQEVVS
QLPSRDGHRLQVWKLDDSYSYDDRVQIVRDLHSWDENKLSSFKKTSFEMT
FLENQIEVSHIPNGLYYVRSIIQTDAVSYPAEFLFEMTDQTVEPLVIVAK
KTDTMTTKVKLIKVDQDHNRLEGVGFKLVSVARDGSEKEVPLIGEYRYSS
SGQVGRTLYTDKNGETFVTNLPLGNYRFKEVEPLAGYAVTTLDTDVQLVD
HQLVTITVVNQKLPRGNVDFMKVDGRTNTSLQGANFKVMKEESGHYTPVL
QNGKEVVVTSGKDGRFRVEGLEYGTYYLWELQAPTGYVQLTSPVSFTIGK
DTRKELVTVVKNNKRPRIDVPDTGEETLYILMLVAILLFGSGYYLTKKPN
N

Orf6_{—}670 is a sortase. An example of an amino acid sequence of orf6_{—}670 is set forth in SEQ ID NO:176.

SEQ ID NO: 176

MLIKMVKTKKQKRNNLLLGVVFFIGMAVMAYPLVSRLYYRVESNQQIADF
DKEKATLDEADIDERMKLAQAFNDSLNNVVSGDPWSEEMKKKGPAEYARM
LEIHERMGHVEIPVIDVDLPVYAGTAEEVLQQGAGHLEGTSLPIGGNSTH
AVITAHTGLPTAKMFTDLTKLKVGDKFYVHNIKEVMAYQVDQVKVIEPTN
FDDLLIVPGHDYVTLLTCTPYMINTHRLLVRGHRIPYVAEVEEEFIAANK
LSHLYRYLFYVAVGLIVILLWIIRRLRKKKKQPEKALKALKAARKEVKVE
DGQQ

Orf7_{—}670 is a sortase. An example of an amino acid sequence of orf7_{—}670 is set forth in SEQ ID NO:177.

SEQ ID NO: 177

VSRYYYRIESNEVIKEFDETVSQMDKAELEERWRLAQAFNATLKPSEILD
PFTEQEKKKGVSEYANMLKVHERIGYVEIPAIDQEIPMYVGTSEEILQKG
AGLLEGASLPVGGENTHTVVTAHRGLPTAELFSQLDKMKKGDVFYLHVLD
QVLAYQVDQILTVEPNDFEPVLIQHGEDYATLLTCTPYMINSHRLLVRGK
RIPYTAPIAERNRAVRERGQFWLWLLLAALVMILVLSYGVYRHRRIVKGL
EKQLEEHHVKG

Orf8_{—}670 is a sortase. An example of an amino acid sequence of orf8_{—}670 is set forth in SEQ ID NO:178.

SEQ ID NO: 178

MSKAKLQKLLGYLLMLVALVIPVYCFGQMVLQSLGQVKGHEIFSESVTAD
SYQEQLQRSLDYNQRLDSQNRIVDPFLAEGYEVNYQVSDDPDAVYGYLSI
PSLEIMEPVYLGADYHHLAHGLAHVDGTPLPVEGKGIRSVIAGHRAEPSH
VFFRHLDQLKVGDALYYDNGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFLGILFVLWKLARLLRGK

As discussed above, a S. pneumoniae AI sequence is present in the 19A Hungary 6 S. pneumoniae genome. Examples of S. pneumoniae AI sequences from 19A Hungary 6 are set forth below.

ORF2_{—}19AH is a transcriptional regulator. An example of an amino acid sequence of ORF2_{—}19AH is set forth in SEQ ID NO:187.

SEQ ID NO: 187

MLNKYIEKRITDKITILNILLDIRSIELDELSTLTSLQSKSLLSILQELQ
ETFEEELTFNLDTQQVQLIEHHSHQTNYYFHQLYNQSTILKILRFFLLQG
NQSFNEFTQKEYISIATGYRVRQKCGLLLRSVGLDLVKNQVVGPEYRIRF
LIALLQFHFGIEIYDLNDGSMDWVTHMIVQSNSQLSHELLEITPDEYVHF
SILVALTWKRREFPLEFPESKEFEKLKNLFMYPILMEHCQTYLEPHANMT
FTQEELDYIFLVYCSANSSFSKDKWNQEKKTHTIQLILQHTRGKHLLSKF
KNILGNDISNSLSFLTALTFLTRTFLFGLQNLVPYYNYYEHYGIESDKPL
YHISKAIVQEWMTEQKIEGVIDQHRLYLFSLYLTETIFSSLPAIPIFIIL
NNQADVNLIKSIILRNFTDKVASVTGYNILISPPPSEEHLTEPLIIITTK
EYLPYVKKQYPKGKHHFLTIALDLHVSQQRLIYOTIVDIRKEAFDKRVAM
IAKKAHYLL

ORF3_{—}19AH is a cell wall surface protein. An example of an amino acid sequence of ORF3_{—}19AH is set forth in SEQ ID NO:188.

SEQ ID NO: 188

MKKVRKIFQKAVAGLCCISQLTAFSSIVALAETPETSPAIGKVVIKETGE
GGALLGDAVFELKNNTDGTTVSQRTEAQTGEAIFSNIKPGTYTLTEAQPP
VGYKPSTKQWTVEVEKNGRTTVQGEQVENREEALSDQYPQTGTYPDVQTP
YQIIKVDGSEKNGQHKALNPNPYERVIPEGTLSKRIYQVNNLDDNQYGIE
LTVSGKTTVETKEASTPLDVVILLDNSNSMSNIRHNHAHRAEKAGEATRA
LVDKITSNPDNRVALVTYGSTIFDGSEATVEKGVADANGKILNDSALWTF
DRTTFTAKTYNYSFLNLTSDPTDIQTIKDRIPSDAEELNKDKLMYQFGAT
FTQKALMTADDILTKQARPNSKKVIFHITDGVPTMSYPINFKYTGTTQSY
RTQLNNFKAKTPNSSGILLEDFVTWSADGEHKIVRGDGESYQMFTKKPVT
DQYGVHQILSITSMEQRAKLVSAGYRFYGTDLYLYWRDSILAYPFNSSTD
WITNHGDPTTWYYNGNMAQDGYDVFTVGVGVNGDPGTDEATATRFMQSIS
SSPDNYTNVADPSQILQELNRYFYTIVNEKKSIENGTITDPMGELIDFQL
GADGRFDPADYTLTANDGSSLVNNVPTGGPQNDGGLLKNAKVFYDTTEKR
IRVTGLYLGTGEKVTLTYNVRLNDQFVSNKFYDTNGRTTLHPKEVEKNTV
RDFPIPKIRDVRKYPEITIPKEKKLGEIEFIKINKNDKKPLRDAVFSLQK
QHPDYPDIYGAIDQNGTYQNVRTGEDGKLTFKNLSDGKYRLFENSEPAGY
KPVQNKPIVAFQIVNGEVRDVTSIVPQDIPAGYEFTNDKHYITNEPIPPK
REYPRTGGIGMLPFYLIGCMMMGGVLLYTRKNP

ORF4_{—}19AH is a cell wall surface protein. An example of an amino acid sequence of ORF4_{—}19AH is set forth in SEQ ID NO:189.

SEQ ID NO: 189

MKSINKFLTMLAALLLTASSLFSAATVFAADNVSTAPDAVTKTLTIHKLL
LSEDDLKTWDTNGPKGYDGTQSSLKDLTGVVAEEIPNVYFELQKYNLTDG
KEKENLKDDSKWTTVHGGLTTKDGLKIETSTLKGVYRIREDRTKTTYVGP
NGQVLTGSKAVPALVTLPLVNNNGTVIDAHVFPKNSYNKPVVDKRIADTL
NYNDQNGLSIGTKIPYVVNTTIPSNATFATSFWSDEMTEGLTYNEDVTIT
LNNVAMDQADYEVTKGXNGFNLKLTEAGLAKINGKDADQKIQITYSATLN
SLAVADIPESNDITYHYGNHQDHGNTPKPTKPNNGQITVTKTWDSQPAPE
GVKATVQLVNAKTGEKVGAPVELSENNWTYTWSGLDNSIEYKVEEEYNGY
SAEYTVESKGKLGVKNWKDNNPAPINPEEPRVKTYGKKFVKVDQKDTRLE
NAQFVVKKADSNKYIAFKSTAQQAADEKAAATAKQKLDAAVAAYTNAADK
QAAQALVDQAQQEYNVAYKEAKFGYVEVAGKDEAMVLTSNTDGQFQISGL
AAGTYKLEEIKAPEGFAKIDDVEFVVGAGSWNQGEFNYLKDVQKNDATKV
VNKKITIPQTGGIGTIIFAVAGAAIMGIAVYAYVKNNKDEDQLA

ORF5_{—}19AH is a cell wall surface protein. An example of an amino acid sequence of ORF5_{—}19AH is set forth in SEQ ID NO:190.

SEQ ID NO: 190

MTMQKMQKMISRIFFVMALCFSLVWGAHAVQAQEDHTLVLQLENYQEVVS
QLPSRDGHRLQVWKLDDSYSYDDRVQIVRDLHSWDENKLSSFKKTSFEMT
FLENQIEVSHIPNGLYYVRSIIQTDAVSYPAEFLFEMTDQTVEPLVIVAK
KTDTMTTKVKLIKVDQDHNRLEGVGFKLVSVARDGSEKEVPLIGEYRYSS
SGQVGRTLYTDKNGEIFVTNLPLGNYRFKEVEPLAGYAVTTLDTDVQLVD
HQLVTITVVNQKLPRGNVDFMKVDGRTNTSLQGAMFKVMKEESGHYTPVL
QNGKEVVVTSGKDGRFRVEGLEYGTYYLWELQAPTGYVQLTSPVSFTIGK
DTRKELVTVVKNNKRPRIDVPDTGEETLYILMLVAILLFGSGYYLTKKPN
N

ORF6_{—}19AH is a putative sortase. An example of an amino acid sequence of ORF6_{—}19AH is set forth in SEQ ID NO:191.

SEQ ID NO: 191

MLIKMVKTKKQKRNNLLLGVVFFIGMAVMAYPLVSRLYYRVESNQQIADF
DKEKATLDEADIDERMKLAQAFNDSLNNVVSGDPWSEEMKKKGRAEYARM
LEIHERMGHVEIPVIDVDLPVYAGTAEEVLQQGAGHLEGTSLPIGGNSTH
AVITAHTGLPTAKMFTDLTKLKVGDKFYVHNIKEVMAYQVDQVKVIEPTN
FDDLLIVPGHDYVTLLTCTPYMINTHRLLVRGHRIPYVAEVEEEFIAANK
LSHLYRYLFYVAVGLIVILLWIIRRLRKKKKQPEKALKALKAARKEVKVE
DGQQ

ORF7_{—}19AH is a putative sortase. An example of an amino acid sequence of ORF7_{—}19AH is set forth in SEQ ID NO:192.

SEQ ID NO: 192

MDNSRRSRKKGTKKKKHPLILLLIFLVGFAVAIYPLVSRYYYRIESNEVI
KEFDETVSQMDKAELEERWRLAQAFNATLKPSEILDPFTEQEKKKGVSEY
ANMLKVHERIGYVEIPAIDQEIPMYVGTSEEILQKGAGLLEGASLPVGGE
NTHTVVTAHRGLPTAELFSQLDKMKKGDVFYLHVLDQVLAYQVDQILTVE
PNDFEPVLIQHGEDYATLLTCTPYMINSHRLLVRGKRIPYTAPIAERNRA
VRERGQFWLWLLLAALVMILVLSYGVYRHRRIVKGLEKQLEEHHVK

ORF8_{—}19AH is a putative sortase. An example of an amino acid sequence of ORF8_{—}19AH is set forth in SEQ ID NO:193.

SEQ ID NO: 193

MSKAKLQKLLGYLLMLVALVIPVYCFGQMVLQSLGQVKGHEIFSESVTAD
SYQEQLQRSLDYNQRLDSQNRIVDPFLAEGYEVNYQVSDDPDAVYGYLSI
PSLEIMEPVYLGADYHHLAMGLAHVDGTPLPVEGKGIRSVIAGHRAEPSH
VFFRHLDQLKVGDALYYDNGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFMGILFVLWKLARLLRGK

As discussed above, a S. pneumoniae AI sequence is present in the 6B Finland 12 S. pneumoniae genome. Examples of S. pneumoniae AI sequences from 6B Finland 12 are set forth below.

ORF2_{—}6BF is a transcriptional regulator. An example of an amino acid sequence of ORF2_{—}6BF is set forth in SEQ ID NO:194.

SEQ ID NO: 194

MLNKYIEKRITDKITILNILLDIRSIELDELSTLTSLQSKSLLSILQELQ
ETFEEELTFNLDTQQVQLIEHHSHQTNYYFHQLYNQSTILKILRFFLLQG
NQSFNEFTQKEYISIATGYRVRQKCGLLLRSVGLDLVKNQVVGPEYRIRF
LIALLQFHFGIEIYDLNDGSMDWVTHMIVQSNSQLSHELLEITPDEYVHF
SILVALTWKRREFPLEFPESKEFEKLKNLFMYPILMEHCQTYLEPHANMT
FTQEELDYIFLVYCSANSSFSKDKWNQEKKTHTIQLILQHTRGKHLLSKF
KNILGNDISNSLSFLTALTFLTRTFLFGLQNLVPYYNYYEHYGIESDKPL
YHISKAIVQEWMTEQKIEGVIDQHRLYLFSLYLTETIFSSLPAIPIFIIL
NNQADVNLIKSIILRNFTDKVASVTGYNILISPPPSEEHLTEPLIIITTK
EYLPYVKKQYPKGKHHFLTIALDLHVSQQRLIYQTIVDIRKEAFDKRVAM
IAKKAHYLL

ORF3_{—}6BF is a cell wall surface protein. An example of an amino acid sequence of ORF3_{—}6BF is set forth in SEQ ID NO:195.

SEQ ID NO: 195

MKKVRKIFQKAVAGLCCISQLTAFSSIVALAETPETSPAIGKVVIKETGE
GGALLGDAVFELKNNTDGTTVSQRTEAQTGEAIFSNIKPGTYTLTEAQPP
VGYKPSTKQWTVEVEKNGRTTVQGEQVENREEALSDQYPQTGTYPDVQTP
YQIIKVDGSEKNGQHKALNPNPYERVIPEGTLSKRIYQVNNLDDNQYGIE
LTVSGKTTVETKEASTPLDVVILLDNSNSMSNIRHNHAHRAEKAGEATRA
LVDKITSNPDNRVALVTYGSTIFDGSEATVEKGVADANGKILNDSALWTF
DRTTFTAKTYNYSFLNLTSDPTDIQTIKDRIPSDAEELNKDKLMYQFGAT
FTQKALMTADDILTKQARPNSKKVIFHITDGVPTMSYPINFKYTGTTQSY
RTQLNNFKAKTPNSSGILLEDFVTWSADGEHKIVRGDGESYQMFTKKPVT
DQYGVHQILSITSMEQRAKLVSAGYRFYGTDLYLYWRDSILAYPFNSSTD
WITNHGDPTTWYYNGNMAQDGYDVFTVGVGVNGDPGTDEATATRFMQSIS
SSPDNYTNVADPSQILQELNRYFYTIVNEKKSIENGTITDPMGELIDFQL
GADGRFDPADYTLTANDGSSLVNNVPTGGPQNDGGLLKNAKVFYDTTEKR
IRVTGLYLGTGEKVTLTYNVRLNDQFVSNKFYDTNGRTTLHPKEVEKNTV
RDFPIPKIRDVRKYPEITIPKEKKLGEIEFIKINKNDKKPLRDAVFSLQK
QHPDYPDIYGAIDQNGTYQNVRTGEDGKLTFKNLSDGKYRLFENSEPAGY
KPVQNKPIVAFQIVNGEVRDVTSIVPQDIPAGYEFTNDKHYITNEPIPPK
REYPRTGGIGMLPFYLIGCMMMGGVLLYTRKHP

ORF4_{—}6BF is a cell wall surface protein. An example of an amino acid sequence of ORF4_{—}6BF is set forth in SEQ ID NO:196.

SEQ ID NO: 196

MKSINKFLTMLAALLLTASSLFSAATVFAADNVSTAPDAVTKTLTIHKLL
LSEDDLKTWDTNGPKGYDGTQSSLKDLTGVVAEEIPNVYFELQKYNLTDG
KEKENLKDDSKWTTVHGGLTTKDGLKIETSTLKGVYRIREDRTKTTYVGP
NGQVLTGSKAVPALVTLPLVNNNGTVIDAHVFPKNSYNKPVVDKRIADTL
NYNDQNGLSIGTKIPYVVNTTIPSNATFATSFWSDEMTEGLTYNEDVTIT
LNNVAMDQADYEVTKGNNGFNLKLTEAGLAKINGKDADQKIQITYSATLN
SLAVADIPESNDITYHYGNHQDHGNTPKPTKPNNGQITVTKTWDSQPAPE
GVKATVQLVNAKTGEKVGAPVELSENNWTYTWSGLDNSIEYKVEEEYNGY
SAEYTVESKGKLGVKNWKDNNPAPINPEEPRVKTYGKKFVKVDQKDTRLE
NAQFVVKKADSNKYIAFKSTAQQAADEKAAATAKQKLDAAVAAYTNAADK
QAAQALVDQAQQEYNVAYKEAKFGYVEVAGKDEAMVLTSNTDGQFQISGL
AAGTYKLEEIKAPEGFAKIDDVEFVVGAGSWNQGEFNYLKDVQKNDATKV
VNKKITIPQTGGIGTIIFAVAGAAIMGIAVYAYVKNNKDEDQLA

ORF5_{—}6BF is a cell wall surface protein. An example of an amino acid sequence of ORF5_{—}6BF is set forth in SEQ ID NO:197.

SEQ ID NO: 197

MTMQKMQKMISRIFFVMALCFSLVWGAHAVQAQEDHTLVLQLENYQEVVS
QLPSRDGHRLQVWKLDDSYSYDDRVQIVRDLHSWDENKLSSFKKTSFEMT
FLENQIEVSHIPNGLYYVRSIIQTDAVSYPAEFLFEMTDQTVEPLVIVAK
KTDTMTTKVKLIKVDQDHNRLEGVGFKLVSVARDGSEKEVPLIGEYRYSS
SGQVGRTLYTDKNGEIFVTNLPLGNYRFKEVEPLAGYAVTTLDTDVQLVD
HQLVTITVVNQKLPRGNVDFMKVDGRTNTSLQGAMFKVMKEESGHYTPVL
QNGKEVVVTSGKDGRFRVEGLEYGTYYLWELQAPTGYVQLTSPVSFTIGK
DTRKELVTVVKNNKRPRIDVPDTGEETLYILMLVAILLFGSGYYLTKKPN
N

ORF6_{—}6BF is a putative sortase. An example of an amino acid sequence of ORF6_{—}6BF is set forth in SEQ ID NO:198.

SEQ ID NO: 198

MLIKMVKTKKQKRNNLLLGVVFFIGMAVMAYPLVSRLYYRVESNQQIADF
DKEKATLDEADIDERMKLAQAFNDSLNNVVSGDPWSEEMKKKGRAEYARM
LEIHERMGHVEIPVIDVDLPVYAGTAEEVLQQGAGHLEGTSLPIGGNSTH
AVITAHTGLPTAKMFTDLTKLKVGDKFYVHNIKEVMAYQVDQVKVIEPTN
FDDLLIVPGHDYVTLLTCTPYMINTHRLLVRGHRIPYVAEVEEEFIAANK
LSHLYRYLFYVAVGLIVTLLWIIRRLRKKKKQPEKALKALKAARKEVKVE
DGQQ

ORF7_{—}6BF is a putative sortase. An example of an amino acid sequence of ORF7_{—}6BF is set forth in SEQ ID NO:199.

SEQ ID NO: 199

MDNSRRSKKGTKKKKKHPLILLLIFLVGFAVAIYPLVSRYYYRIESNEVI
KEFDETVSQMDKAELEERWRLAQAFNATLKPSEILDPFTEQEKKKGVSEY
ANMLKVHERIGYVEIPAIDQEIPMYVGTSEEILQKGAGLLEGASLPVGGE
NTHTVVTAHRGLPTAELFSQLDKMKKGDVFYLHVLDQVLAYQVDQILTVE
PNDFEPVLIQHGEDYATLLTCTPYMINSHRLLVRGKRIPYTAPIAERNRA
VRERGQFWLWLLLAALVMILVLSYGVYRHRRIVKGLEKQLEEHHVKG

ORF8_{—}6BF is a putative sortase. An example of an amino acid sequence of ORF8_{—}6BF is set forth in SEQ ID NO:200.

SEQ ID NO: 200

MSKAKLQKLLGYLLMLVALVIPVYCFGQMVLQSLGQVKGHEIFSESVTAD
SYQEQLQRSLDYNQRLDSQNRIVDPFLAEGYEVNYQVSDDPDAVYGYLSI
PSLEIMEPVYLGADYHHLAMGLAHVDGTPLPVEGKGIRSVIAGHRAEPSH
VFFRHLDQLKVGDALYYDNGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFLGILFVLWKLARLLRGK

As discussed above, a S. pneumoniae AI sequence is present in the 6B Spain 2 S. pneumoniae genome. Examples of S. pneumoniae AI sequences from 6B Spain 2 are set forth below.

ORF2_{—}6BSP is a transcriptional regulator. An example of an amino acid sequence of ORF2_{—}6BSP is set forth in SEQ ID NO:201.

SEQ ID NO: 201

MLNKYIEKRITDKITILNILLDIRSIELDELSTLTSLQSKSLLSILQELQ
ETFEEELTFNLDTQQVQLIEHHSHQTNYYFQHLYNQSTILKILRFFLLQG
NQSFNEFTQKEYISIATGYRVRQKCGLLLRSVGLDLVKNQVVGPEYRIRF
LIALLQFHFGIEIYDLNDGSMDWVTHMIVQSNSQLSHELLEITPDYRVHF
SILVALTWKRREFPLEFPESKEFEKLKNLFMYPILMEHCQTYLEPHANMT
FTQEELDYIFLVYCSANSSFSKDKWNQEKKTHTIQLILQHTRGHKLLSKF
KNILGNDISNSLSFLTALTFLTRTFLFGLQNLVPYYNYYEHYGIESDKPL
YHISKAIVQEWMTEQKIEGVIDQHRLYLFSLYLTETIFSSLPAIPIFIIL
NNQADVNLIKSIILRNFTDKVASVTGYNILISPPPSEEHLTEPLIIITTK
EYLPYVKKQYPKGKHHFLTIALDLHVSQQRLIYQTIVDIRKEAFDKRVAM
IAKKAHYLL

ORF3_{—}6BSP is a cell wall surface protein. An example of an amino acid sequence of ORF3_{—}6BSP is set forth in SEQ ID NO:202.

SEQ ID NO: 202

MKKVRKIFQKAVAGLCCISQLTAFSSIVALAETPETSPAIGKVVIKETGE
GGALLGDAVFELKNNTDGTTVSQRTEAQTGEAIFSNIKPGTYTLTEAQPP
VGYKPSTKQWTVEVEKNGRTTVQGEQVENREEALSDQYPQTGTYPDVQTP
YQIIKVDGSEKNGQHKALNPNPYERVIPEGTLSKRIYQVNNLDDNQYGIE
LTVSGKTTVETKEASTPLDVVILLDNSNSMSNIRHNHAHRAEKAGEATRA
LVDKITSNPDNRVALVTYGSTIFDGSEATVEKGVADANGKILNDSALWTF
DRTTFTAKTYNYSFLNLTSDPTDIQTIKDRIPSDAEELNKDKLMYQFGAT
FTQKALMTADDILTKQARPNSKKVIFHITDGVPTMSYPINFKYTFTTQSY
RTQLNNFKAKTPNSSGILLEDFVTWSADGEHKIVRGDGESYQMFTKKPVT
DQYGVHQILSITSMEQRAKLVSAGYRFYGTDLYLYWRDSILAYPFNSSTD
WITNHGDPTTWYYNGNMAQDGYDVFTVGVGVNGDPGTDEATATRFMQSIS
SSPDNYTNVADPSQILEALNRYFYTIVNEKKSIENGTITDPMGELIDFQL
GADGRFDPADYTLTANDGSSLVNNVPTGGPQNDGGLLKNAKVFYDTTEKR
IRVTGLYLGTGEKVTLTYNVRLNDQFVSNKFYDTNGRTTLHPKEVEKNTV
RDFPIPKIRDVRKYPEITIPKEKKLGEIEFIKINKNDKKPLRDAVFSLQK
QHPDYPDIYGAIDQNGTYQNVRTGEDGKLTFKNLSDGKYRLFENSEPAGY
KPVQNKPIVAFQIVNGEVRDVTSIVQPDIPAGYEFTNDKHYITNEPIPPK
REYPRTGGIGMLPFYLIGCMMMGGVLLYTRKHP

ORF4_{—}6BSP is a cell wall surface protein. An example of an amino acid sequence of ORF4_{—}6BSP is set forth in SEQ ID NO:203.

SEQ ID NO: 203

MKSINKFLTMLAALLLTASSLFSAATVFAADNVSTAPDAVTKTLTIHKLL
LSEDDLKTWDTNGPKGYDGTQSSLKDLTGVVAEEIPNVYFELQKYNLTDG
KEKENLKDDSKWTTVHGGLTTKDGLKIETSTLKGVYRIREDRTKTTYVGP
NGQVLTGSKAVPALVTLPLVNNNGTVIDAHVFPKNSYNKPVVDKRIADTL
NYNDQNGLSIGTKIPYVVNTTIPSNATFATSFWSDEMTEGLTYNEDVTIT
LNNVAMDQADYEVTKGNNGFNLKLTEAGLAKINGKDADQKIQITYSATLN
SLAVADIPESNDITYHYGNHQDHGNTPKPTKPNNGQITVTKTWDSQPAPE
GVKATVQLVNAKTGEKVGAPVELSENNWTYTWSGLDNSIEYKVEEEYNGY
SAEYTVESKGKLGVKNWKDNNPAPINPEEPRVKTYGKKFVKVDQKDTRLE
NAQFVVKKADSNKYIAFKSTAQQAADEKAAATAKQKLDAAVAAYTNAADK
QAAQALVDQAQQEYNVAYKEAKFGYVEVAGKDEAMVLTSNTDGQFQISGL
AAGTYKLEEIKAPEGFAKIDDVEFVVGAGSWNQGEFNYLKDVQKNDATKV
VNKKITIPQTGGIGTIIFAVAGAAIMGIAVYAYVKNNKDEDQLA

ORF5_{—}6BSP is a cell wall surface protein. An example of an amino acid sequence of ORF5_{—}6BSP is set forth in SEQ ID NO:204.

SEQ ID NO: 204

MTMQKMQKMISRIFFVMALCFSLVWGAHAVQAQEDHTLVLQLENYQEVVS
QLPSRDGHRLQVWKLDDSYSYDDRVQIVRDLHSWDENKLSSFKKTSFEMT
FLENQIEVSHIPNGLYYVRSIIQTDAVSYPAEFLFEMTDQTVEPLVIVAK
KTDTMTTKVKLIKVDQDHNRLEGVGFKLVSVARDGSEKEVPLIGEYRYSS
SGQVGRTLYTDKNGEIFVTNLPLGNYRFKEVEPLAGYAVTTLDTDVQLVD
HQLVTITVVNQKLPRGNVDFMKVDGRTNTSLQGAMFKVMKEESGHYTPVL
QNGKEVVVTSGKDGRFRVEGLEYGTYYLWELQAPTGYVQLTSPVSFTIGK
DTRKELVTVVKNNKRPRIDVPDTGEETLYILMLVAILLFGSGYYLTKKPN
N

ORF6_{—}6BSP is a putative sortase. An example of an amino acid sequence of ORF6_{—}6BSP is set forth in SEQ ID NO:205.

SEQ ID NO: 205

MLIKMVKTKKQKRNNLLLGVVFFIGMAVMAYPLVSRLYYRVESNQQIADF
DKEKATLDEADIDERMKLAQAFNDSLNNVVSGDPWSEEMKKKGRAEYARM
LEIHERMGHVEIPVIDVDLPVYAGTAEEVLQQGAGHLEGTSLPIGGNSTH
AVITAHTGLPTAKMFTDLTKLKVGDKFYVHNIKEVMAYQVDQVKVIEPTN
FDDLLIVPGHDYVTLLTCTPYMINTHRLLVRGHRIPYVAEVEEEFIAANK
LSHLYRYLFYVAVGLIVILLWIIRRLRKKKKQPEKALKALKAARKEVKVE
DGQQ

ORF7_{—}6BSP is a putative sortase. An example of an amino acid sequence of ORF7_{—}6BSP is set forth in SEQ ID NO:206.

SEQ ID NO: 206

MDNSRRSRKKGTKKKKHPLILLLIFLVGFAVAIYPLVSRYYYRIESNEVI
KEFDETVSQMDKAELEERWRLAQAFNATLKPSEILDPFTEQEKKKGVSEY
ANMLKVHERIGYVEIPAIDQEIPMYVGTSEEILQKGAGLLEGASLPVGGE
NTHTVVTAHRGLPTAELFSQLDKMKKGDVFYLHVLDQVLAYQVDQILTVE
PNDFEPVLIQHGEDYATLLTCTPYMINSHRLLVRGKRIPYTAPIAERNRA
VRERGQFWLWLLLAALVMILVLSYGVYRHRRIVKGLEKQLEEHHVKG

ORF8_{—}6BSP is a putative sortase. An example of an amino acid sequence of ORF8_{—}6BSP is set forth in SEQ ID NO:207.

SEQ ID NO: 207

MSKAKLQKLLGYLLMLVALVIPVYCFGQMVLQSLGQVKGHEIFSESVTAD
SYQEQLQRSLDYNQRLDSQNRIVDPFLAEGYEVNYQVSDDPDAVYGYLSI
PSLEIMEPVYLGADYHHLAMGLAHVDGTPLPVEGKGIRSVIAGHRAEPSH
VFFRHLDQLKVGDALYYDNGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFLGILFVLWKLARLLRGK

As discussed above, a S. pneumoniae AI sequence is present in the 9V Spain 3 S. pneumoniae genome. Examples of S. pneumoniae AI sequences from 9V Spain 3 are set forth below.

ORF2_{—}9VSP is a transcriptional regulator. An example of an amino acid sequence of ORF2_{—}9VSP is set forth in SEQ ID NO:208.

SEQ ID NO: 208

MLNKYIEKRITDKITILNILLDIRSIELDELSTLTSLQSKSLLSILQELQ
ETFEEELTFNLDTQQVQLIEHHSHQTNYYFHQLYNQSTILKILRFFLLQG
NQSFNEFTQKEYISIATGYRVRQKCGLLLRSVGLDLVKNQVVGPEYRIRF
LIALLQFHFGIEIYDLNDGSMDWVTHMIVQSNSQLSHELLEITPDEYVHF
SILVALTWKRREFPLEFPESKEFEKLKNLFMYPILMEHCQTYLEPHANMT
FTQEELDYIFLVYCSANSSFSKDKWNQEKKTHTIQLILQHTRGKHLLSKF
KNILGNDISNSLSFLTALTFLTRTFLFGLQNLVPYYNYYEHYGIESDKPL
YHISKAIVQEWMTEQKIEGVIDQHRLYLFSLYLTETIFSSLPAIPIFIIL
NNQADVNLIKSIILRNFTDKVASVTGYNILISPPPSEEHLTEPLIIITTK
EYLPYVKKQYPKGKHHFLTIALDLHVSQQRLIYQTIVDIRKEAFDKRVAM
IAKKAHYLL

ORF3_{—}9VSP is a cell wall surface protein. An example of an amino acid sequence of ORF3_{—}9VSP is set forth in SEQ ID NO:209.

SEQ ID NO: 209

MKKVRKIFQKAVAGLCCISQLTAFSSIVALAETPETSPAIGKVVIKETGE
GGALLGDAVFELKNNTNGTTVSQRTEAQTGEAIFSNIKPGTYTLTEAQPP
VGYKPSTKQRTVEVEKNGRTTVQGEQVENREEALSDQYPQTGTYPDVQTP
YQIIKVDGSEKNGQHKALNPNPYERVIPEGTLSKRIYQVNNLDDNQYGIE
LTVSGKTVYERKDKSVPLDVVILLDNSNSMSNIRNKNARRAERAGEATRS
LIDKITSDPENRVALVTYASTIFDGTEFTVEKGVADKNGKRLNDSLFWNY
DQTSFTTNTKDYSYLKLTNDKNDIVELKNKVPTEAEDHDGNRLMYQFGAT
FTQKALMKADEILTQQARQNSQKVIFHITDGVPTMSYPINFNHATFAPSY
QNQLNAFFSKSPNKDGILLSDFITQATSGEHTIVRGDGQSYQMFTDKTVY
EKGAPAAFPVKPEKYSEMKAVGYAVIGDPINGGYIWLNWRESILAYPFNS
NTAKITNHGDPTRWYYNGNIAPDGYDVFTGIGINGDPGTDEATATASFMQ
SISSKPENYTNVTDTTKILEQLNRYFHTIVTEKKSIENGTITDPMGELID
LQLGTDGRFDPADYTLTANDGSRLENGQAVGGPQNDGGLLKNAKVFYDTT
EKRIRVTGLYLGTGEKVTLTYNVRLNDQFVSNKFYDTNGRTTLHPKEVEK
NTVRDFPIPKIRDVRKYPAITIAKEKKLGEIEFIKINKNDKKPLRDAVFS
LQKQHPDYPDIYGAIDQNGTYQNVRTGEDGKLTFKNLSDGKYRLFENSEP
AGYKPVQNKPIVAFQIVNGEVRDVTSIVPQDIPAGYEFTNDKHYITNEPI
PPKREYPRTGGIGMLLFYLIGCMMMGGVLLYTRKHP

ORF4_{—}9VSP is a cell wall surface protein. An example of an amino acid sequence of ORF4_{—}9VSP is set forth in SEQ ID NO:210.

SEQ ID NO: 210

MKSINKFLTMLAALLLTASSLFSAATVFAAGTTTTSVTVHKLLATDGDMD
KIANELETGNYAGNKVGVLPANAKEIAGVMFVWTNTNNEIIDENGQTLGV
NIDPQTFKLSGAMPATAMKKLTEAEGAKFNTANLPAAKYKIYEIHSLSTY
VGEDGATLTGSKAVPIEIELPLNDVVDAHVYPKNTEAKPKIDKDFKGKAN
PDTPRVDKDTPVNHQVGDVVEYEIVTKIPALANYATANWSDRMTEGLAFN
KGTVKVTVDDVALEAGDYALTEVATGFDLKLTDAGLAKVNDQNAEKTVKI
TYSATLNDKAIVEVPESNDVTFNYGNNPDHGHTPKPNKPNENGDLTLTKT
WVDATGAPIPAGAEATFDLVNAQTGKVVQTVTLTTDKNTVTVNGLDKNTE
YKFVERSIKGYSADYQEITTAGEIAVKNWKDENPKPLDPTEPKVVTYGKK
FVKVNDKDNRLAGAEFVIANADNAGQYLARKADKVSQEEKQLVVTTKDAL
DRAVAAYNALTAQQQTQQEKEKVDKAQAAYNAAVIAANNAFEWVADKDNE
NVVKLVSDAQGRFEITGLLAGTYYLEETKQPAGYALLTSRQKFEVTATSY
SATGQGIEYTAGSGKDDATKVVNKKITIPQTGGIGTIIFAVAGAVIMGIA
VYAYVKNNKDEDQLA

ORF5_{—}9VSP is a cell wall surface protein. An example of an amino acid sequence of ORF5_{—}9VSP is set forth in SEQ ID NO:211.

SEQ ID NO: 211

MTMQKMQKMQKMQKMQKMQKMISRIFFVMALVFSLVWGAHAVQAQEDHTL
VLQLENYQEVVSQLPSRDGHRLQVWKLDDSYSYDNRVQIVRDLHSWDENK
LSSFKKTSFEMTFLENQIEVSHIPNGLYYVRSIIQTDAVSYPAEFLFEMT
DQTVEPLVIVAKKADTVTTKVKLIKVDQDHNRLEGVGFKLVSVARDGSEK
EVPLIGEYRYSSSGQVGRTLYTDKNGEIVVTNLPLGTYRFKEVEPLAGYT
VTTMDTDVQLVDHQLVTITVVNQKLPRGNVDFMKVDGRTNTSLQGAMFKV
MKEENGHYTPVLQNGKEVVVASGKDGRFRVEGLEYGTYYLWELQAPTGYV
QLTSPVSFTIGKDTRKELVTVVKNNKRPRIDVPDTGEETLIYLMLVAILL
FGSGYYLTKKTNN

ORF6_{—}9VSP is a putative sortase. An example of an amino acid sequence of ORF6_{—}9VSP is set forth in SEQ ID NO:212.

SEQ ID NO: 212

MLIKMAKTKKQKRNNLLLGVVFFIGIAVMAYPLVSRLYYRVESNQQIADF
DKEKATLDEADIDERMKLAQAFNDSLNNVVSGDPWSEEMKKKGRAEYARM
LEIHERMGHVEIPAIDVDLPVYAGTAEEVLQQGAGHLEGTSLPIGGNSTH
AVITAHTGLPTAKMFTDLTKLKVGDKFYVHNIKEVMAYQVDQVKVIEPTN
FDDLLIVPGHDYVTLLTCTPYMINTHRLLVRGHRIPYVAEVEEEFIAANK
LSHLYRYLFYVAVGLIVILLWIIRRLRKKKRQSERALKALKEATKEVKVE
DE

ORF7_{—}9VSP is a putative sortase. An example of an amino acid sequence of ORF7_{—}9VSP is set forth in SEQ ID NO:213.

SEQ ID NO: 213

MSKSRYSRKKSVKKKKNPFILLLIFLVGLAVAMYPLVSRYYYRIESNEVI
KEFDETVSQMDKAELEERWRLAQAFNATLKPSEILDPFTEQEKKKGVSEY
ANMLKVHERIGYVEIPAIDQEIPMYVGTSEEILQKGAGLLEGASLPVGGE
NTHTVVTAHRGLPTAELFSQLDKMKKGDIFYLHVLDQVLAYQVDQIVTVE
PNDFEPVLIQHGEDYATLLTCTPYMINSHRLLVRGKRIPYTAPIAERNRA
VRERGQFWLWLLLGAMAVILLLLYRVYRNRRIVKGLEKQLEGRHVKD

ORF8_{—}9VSP is a putative sortase. An example of an amino acid sequence of ORF8_{—}9VSP is set forth in SEQ ID NO:214.

SEQ ID NO: 214

MSRTKLRALLGYLLMLVACLIPIYCFGQMVLQSLGQVKGHATFVKSMTTE
MYQEQQNHSLAYNQRLASQNRIVDPFLAEGYEVNYQVSDDPDAVYGYLSI
PSLEIMEPVYLGAGYHHLGMGLAHVDGTPLPLDGTGIRSVIAGHRAEPSH
VFFRHLDQLKVGDALYYDNGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFLGILFVLWKLARLLRGK

As discussed above, a S. pneumoniae AI sequence is present in the 14 CSR 10 S. pneumoniae genome. Examples of S. pneumoniae AI sequences from 14 CSR 10 are set forth below.

ORF2_{—}14CSR is a transcriptional regulator. An example of an amino acid sequence of ORF2_{—}14CSR is set forth in SEQ ID NO:215.

SEQ ID NO: 215

MLNKYIEKRITDKITILNILLDIRSIELDELSTLTSLQSKSLLSILQELQ
ETFEEELTFNLDTQQVQLIEHHSHQTNYYFHQLYNQSTILKILRFFLLQG
NQSFNEFTQKEYISIATGYRVRQKCGLLLRSVGLDLVKNQVVGPEYRIRF
LIALLQFHFGIEIYDLNDGSMDWVTHMIVQSNSQLSHELLEITPDEYVHF
SILVALTWKRREFPLEFPESKEFEKLKNLFMYPILMEHCQTYLEPHANMT
FTQEELDYIFLVYCSANSSFSKDKWNQEKKTHTIQLILQHTRGKHLLSKF
KNILGNDISNSLSFLTALTFLTRTFLFGLQNLVPYYNYYEHYGIESDKPL
YHISKAIVQEWMTEQKIEGVIDQHRLYLFSLYLTETIFSSLPAIPIFIIL
NNQADVNLIKSIILRNFTDKVASVTGYNILISPPPSEEHLTEPLIIITTK
EYLPYVKKQYPKGKHHFLTIALDLHVSQQRLIYQTIVDIRKEAFDKRVAM
IAKKAHYLL

ORF3_{—}14CSR is a cell wall surface protein. An example of an amino acid sequence of ORF3_{—}14CSR is set forth in SEQ ID NO:216.

SEQ ID NO: 216

MKKVRKIFQKAVAGLCCISQLTAFSSIVALAETPETSPAIGKVVIKETGE
GGALLGDAVFELKNNTDGTTVSQRTEAQTGEAIFSNIKPGTYTLTEAQPP
VGYKPSTKQWTVEVEKNGRTTVQGEVQENREEALSDQYPQTGTYPDVQTP
YQIIKVDGSEKNGQHKALNPNPYERVIPEGTLSKRIYQVNNLDDNQYGIE
LTVSGKTTVETKEASTPLDVVILLDNSNSMSNIRHNHAHRAEKAGEATRA
LVDKITSNPDNRVALVTYGSTIFDGSEATVEKGVADANGKILNDSALWTF
DRTTFTAKTYNYSFLNLTSDPTDIQTIKDRIPSDAEELNKDKLMYQFGAT
FTQKALMTADDILTKQARPNSKKVIFHITDGVPTMSYPINFKYTFTTQSY
RTQLNNFKAKTPNSSGILLEDFVTWSADGEHKIVRGDGESYQMFTKKPVT
DQYGVHQILSITSMEQRAKLVSAGYRFYGTDLYLYWRDSILAYPFNSSTD
WITNHGDPTTWYYNGNMAQDGYDVFTVGVGVNGDPGTDEATATRFMQSIS
SSPDNYTNVADPSQILQELNRYFYTIVNEKKSIENGTITDPMGELIDFQL
GADGRFDPADYTLTANDGSSLVNNVPTGGPQNDGGLLKNAKVFYDTTEKR
IRVTGLYLGTGEKVTLTYNVRLNDQFVSNKFYDTNGRTTLHPKEVEKNTV
RDFPIPKIRDVRKYPEITIPKEKKLGEIEFIKINKNDKKPLRDAVFSLQK
QHPDYPDIYGAIDQNGTYQNVFTGEDGKLTFKNLSDGKYRLFENSEPAGY
KPVQNKPIVAFQIVNGEVRDVTSIVPQDIPAGYEFTNDKHYITNEPIPPK
REYPRTGGIGMLPFYLIGCMMMGGVLLYTRKHP

ORF4_{—}14CSR is a cell wall surface protein. An example of an amino acid sequence of ORF4_{—}14CSR is set forth in SEQ ID NO:217.

SEQ ID NO: 217

MKSINKFLTMLAALLLTASSLFSAATVFAADNVSTAPDAVTKTLTIHKLL
LSEDDLKTWDTNGPKGYDGTQSSLKDLTGVVAEEPINVYFELQKYNLTDG
KEKENLKDDSKWTTVHGGLTTKDGLKIETSTLKGVYRIREDRTKTTYVGP
NGQVLTGSKAVPALVTLPLVNNNGTVIDAHVFPKNSYNKPVVDKRIADTL
NYNDQNGLSIGTKIPYVVNTTIPSNATFATSFWSDEMTEGLTYNEDVTIT
LNNVAMDQADYEVTKGNNGFNLKLTEAGLAKINGKDADQKIQITYSATLN
SLAVADIPESNDITYHIGNHQDHGNTPKPTKPNNGQITVTKTWDSQPAPE
GVKATVQLVNAKTGEKVGAPVELSENNWTYTWSGLDNSIEYKVEEEYNGY
SAEYTVESKGKLGVKNWKDNNPAPINPEEPRVKTYGKKFVKVDQKDTRLE
NAQFVVKKADSNKYIAFKSTAQQAADEKAAATAKQKLDAAVAAYTNAADK
QAAQALVDQAQQEYNVAYKEAKFGYVEVAGKDEAMVLTSNTDGQFQISGL
AAGTYKLEEIKAPEGFAKIDDVEFVVGAGSWNQGEFNYLKDVQKNDATKV
VNKKITIPQTGGIGTIIFAVAGAAIMGIAVYAYVKNNKDEDQLA

ORF5_{—}14CSR is a cell wall surface protein. An example of an amino acid sequence of ORF5_{—}14CSR is set forth in SEQ ID NO:218.

SEQ ID NO: 218

MTMQKMQKMISRIFFVMALVFSLVWGAHAVQAQEDHTLVLQLENYQEVVS
QLPSRDGHRLQVWKLDDSYSYDDRVQIVRDLHSWDENKLSSFKKTSFEMT
FLENQIEVSHIPNGLYYVRSIIQTDAVSYPAEFLFEMTDQTVEPLVIVAK
KTDTMTTKVKLIKVDQDHNRLEGVGFKLVSVARDGSEKEVPLIGEYRYSS
SGQVGRTLYTDKNGEIFVTNLPLGNYRFKEVEPLAGYAVTTLDTLVQLVD
HQLVTITVVKQKLPRGNVDFMKVDGRTNTSLQGAMFKVMKEESGHYTPVL
QNGKEVVVTSGKDGRFRVEGLEYGTYYLWELQAPTGYVQLTSPVSFTIGK
DTRKELVTVVKNNKRPRIDVPDTGEETLYILMLVAILLFGSGYYLTKKPN
N

ORF6_{—}14CSR is a putative sortase. An example of an amino acid sequence of ORF6_{—}14CSR is set forth in SEQ ID NO:219.

SEQ ID NO: 219

MLIKMVKTKKQKRNNLLLGVVFFIGMAVMAYPLVSRLYYRVESNQQIADF
DKEKATLDEADIDERMKLAQAFNDSLNNVVSGDPWSEEMKKKGRAEYARM
LEIHERMGHVEIPVIDVDLPVYAGTAEEVLQQGAGHLEGTSLPIGGNSTH
AVITAHTGLPTAKMFTDLTKLKVGDKFYVHNIKEVMAYQVDQVKVIEPTN
FDDLLIVPGHDYVTLLTCTPYMINTHRLLVRGHRIPYVAEVEEEFIAANK
LSHLYRYLFYVAVGLIVILLWIIRRLRKKKKQPEKALKALKAARKEVKVE
DGQQ

ORF7_{—}14CSR is a putative sortase. An example of an amino acid sequence of ORF7_{—}14CSR is set forth in SEQ ID NO:220.

SEQ ID NO: 220

MDNSRRSRKKGTKKKKHPLILLLIFLVGFAVAIYPLVSRYYYRIESNEVI
KEFDETVSQMDKAELEERWRLAQAFNATLKPSEILDPFTEQEKKKGVSEY
ANMLKVHERIGYVEIPAIDQEIPMYVGTSEEILQKGAGLLEGASLPVGGE
NTHTVVTAHRGLPTAELFSQLDKMKKGDVFYLHVLDQVLAYQVDQILTVE
PNDFEPVLIQHGEDYATLLTCTPYMINSHRLLVRGKRIPYTAPIAERNRA
VRERGQFWLWLLLAALVMILVLSYGVYRHRRIVKGLEKQLEEHHVKG

ORF8_{—}14CSR is a putative sortase. An example of an amino acid sequence of ORF8_{—}14CSR is set forth in SEQ ID NO:221.

SEQ ID NO: 221

MSKAKLQKLLGYLLMLVALVIPVYCFGQMVLQSLGQVKGHEIFSESVTAD
SYQEQLQRSLDYNQRLDSQNRIVDPFLAEGYEVNYQVSDDPDAVYGYLSI
PSLEIMEPVYLGADYHHLAMGLAHVDGTPLPVEGKGIRSVIAGHRAEPSH
VFFRHLDQLKVGDALYYDNGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFLGILFVLWKLARLLRGK

As discussed above, a S. pneumoniae AI sequence is present in the 19F Taiwan 14 S. pneumoniae genome. Examples of S. pneumoniae AI sequences from 19F Taiwan 14 are set forth below.

ORF2_{—}19FTW is a transcriptional regulator. An example of an amino acid sequence of ORF2_{—}19FTW is set forth in SEQ ID NO:222.

SEQ ID NO: 222

MLNKYIEKRITDKITILNILLDIRSIELDELSTLTSLQSKSLLSILQELQ
ETFEEELTFNLDTQQVQLIEHHSHQTNYYFHQLYNQSTILKILRFFLLQG
NQSFNEFTQKEYISIATGYRVRQKCGLLLRSVGLDLVKNQVVGPEYRIRF
LIALLQFHFGIEIYDLNDGSMDWVTHMIVQSNSQLSHELLEITPDEYVHF
SILVALTWKRREFPLEFPESKEFEKLKNLFMYPILMEHCQTYLEPHANMT
FTQEELDYIFLVYCSANSSFSKDKWNQEKKTHTIQLILQHTRGKHLLSKF
NNILGNDISNSLSFLTALTFLTRTFLFGLQNLVPYYNYYEHYGIESDKPL
YHISKAIVQEWMTEQKIEGVIDQHRLYLFSLYLTETIFSSLPAIPIFIIL
NNQADVNLIKSIILRNFTDKVASVTGYNILISPPPSEEHLTEPLIIITTK
EYLPYVKKQYPKGKHHFLTIALDLHVSQQRLIYQTIVDIRKEAFDKRVAM
IAKKAHYLL

ORF3_{—}19FTW is a cell wall surface protein. An example of an amino acid sequence of ORF3_{—}19FTW is set forth in SEQ ID NO:223.

SEQ ID NO: 223

MKKVRKIFQKAVAGLCCISQLTAFSSIVALAETPETSPAIGKVVIKETGE
GGALLGDAVFELKNNTDGTTVSQRTEAQTGEAIFSNIKPGTYTLTEAQPP
VGYKPSTKQWTVEVEKNGRTTVQGEQVENREEALSDQYPQTGTYPDVQTP
YQIIKVDGSEKNGQHKALMPNPYERVIPEGTLSKRIYQVNNLDDNQYGIE
LTVSGKTVYERKDKSVPLDVVILLDNSNSMSNIRNKNARRAERAGEATRS
LIDKITSDPENRVALVTYASTIFDGTEFTVEKGVADKNGKRLNDSLFWNY
DQTSFTTNTKDYSYLKLTNDKNDIVELKNKVPTEAEDHDGNRLMYQFGAT
FTQKALMKADEILTQQARQNSQKVIFHITDGVPTMSYPINFNHATFAPSY
QNQLNAFFSKSPNKDGILLSDFITQATSGEHTIVRGDGQSYQMFTDKTVY
EKGAPAAFPVKPEKYSEMKAVGYAVIGDPINGGYIWLNWRESILAYPFNS
NTAKITNHGAPTRWYYNGNIAPDGYDVFTVGIGINGDPGTDEATATSFMQ
SISSKPENYTNVTDTTKILEALNRYFHTIVTEKKSIENGTITDPMGELID
LQLGTDGRFDPADYTLTANDGSRLENGQAVGGPQNGDDLLKNAKVFYDTT
EKRIRVTGLYLGTGEKVTLTYNVRLNDQFVSNKFYDTNGRTTLHPKEVEK
NTVRDFPIPKIRDVRKYPAITAIKEKKLGEIEFIKINKNDKKPLRDAVFS
LQKQHPDYPDIYGAIDQNGTYQNVRTGEDGKLTFKNLSDGKYRLFENSEP
AGYKPVQNKPIVAFQIVNGEVRDVTSIVPQDIPAGYEFTNDKHYITNEPI
PPKREYPRTGGIGMLPFYLIGCMMMGGVLLYTRKHP

ORF4_{—}19FTW is a cell wall surface protein. An example of an amino acid sequence of ORF4_{—}19FTW is set forth in SEQ ID NO:224.

SEQ ID NO: 224

MKSINKFLTMLAALLLTASSLFSAATVFAAGTTTTSVTVHKLLATDGDMD
KIANELETGNYAGNKVGVLPANAKEIAGVMFVWTNTNNEIIDENGQTLGV
NIDPQTFKLSGAMPATAMKKLTEAEGAKFNTANLPAAKYKIYEIHSLSTY
VGEDGATLTGSKAVPIEIELPLNDVVDAHVYPKNTEAKPKIDKDFKGKAN
PDTPRVDKDTPVNHQVGDVVEYEIVTKIPALANYATANWSDRMTEGLAFN
KGTVKVTVDDVALEAGDYALTEVATGFDLKLTDAGLAKVNDQNAEKTVKI
TYSATLNDKAIVEVPESNDVTFNYGNNPDHGNTPKPNKPNENGDLTLTKT
WVDATGAPIPAGAEATFDLVNAQTGKVVQTVTLTTDKNTVTVNGLDKNTE
YKFVERSIKGYSADYQEITTAGEIAVKNWKDENPKPLDPTEPKVVTYGKK
FVKVNDKDNRLAGAEFVIANADNAGQYLARKADKVSQEEKQLVVTTKDAL
DRAVAAYNALTAQQQTQQEKEKVDKAQAAYNAAVIAANNAFEWVADKDNE
NVVKLVSDAQGRFEITGLLAGTYYLEETKQPAGYALLTSRQKFEVTATSY
SATGQGIEYTAGSGKDDATKVVNKKITIPQTGGIGTIIFAVAGAVIMGIA
VYAYVKNNKDEDQLA

ORF5_{—}19FTW is a cell wall surface protein. An example of an amino acid sequence of ORF5_{—}19FTW is set forth in SEQ ID NO:225.

SEQ ID NO: 225

MTMQKMQKMISRIFFVMALCFSLVWGAHAVQAQEDHTLVLQLENYQEVVS
QLPSRDGHRLQVWKLDDSYSYDNRVQIVRDLHSWDENKLSSFKKTSFEMT
FLENQIEVSHIPNGLYYVRSIIQTDAVSYPAEFLFEMTDQTVEPLVIVAK
KADTVTTKVKLIKVDQDHNRLEGVGFKLVSVARDGSEKEVPLIGEYRYSS
SGQVGRTLYTDKNGEIVVTNLPLGTYRFKEVEPLAGYTVTTMDTDVQLVD
HQLVTITVVNQKLPRGNVDFMKVDGRTNTSLQGAMFKVMKEENGHYTPVL
QNGKEVVVASGKDGRFRVEGLEYGTYYLWELQAPTGYVQLTSPVSFTIGK
DTRKELVTVVKNNKRPRIDVPDTGEETLYILMLVAILLFGSGYYLTKKTN
N

ORF6_{—}19FTW is a putative sortase. An example of an amino acid sequence of ORF6_{—}19FTW is set forth in SEQ ID NO:226.

SEQ ID NO: 226

MLIKMAKTKKQKRNNLLLGVVFFIGMAVMAYPLVSRLYYRVESNQQIADF
DKEKATLDEADIDERMKLAQAFNDSLNNVVSGDPWSEEMKKKGRAEYARM
LEIHERMGHVEIPAIDVDLPVYAGTAEEVLQQGAGHLEGTSLPIGGNSTH
AVITAHTGLPTAKMFTDLTKLKVGDKFYVHNIKEVMAYQVDQVKVEIPTN
FDDLLIVPGHDYVTLLTCTPYMINTHRLLVRGHRIPYVAEVEEEFIAANK
LSHLYRYLFYVAVGLIVILLWIIRRLRKKKRQSERALKALKEATKEVKVE
DE

ORF7_{—}19FTW is a putative sortase. An example of an amino acid sequence of ORF7_{—}19FTW is set forth in SEQ ID NO:227.

SEQ ID NO: 227

MSKSRYSRKKSVKKKKNPFILLLIFLVGALVAMYPLVSRYYYRIESNEVI
KEFDETVSQMDKAELEERWRLAQAFNATLKPSEILDPFTDQEKKQGVSEY
ANMLKVHERIGYVEIPAIEQEIPMYVGTSEDILQKGAGLLEGASLPVGGE
NTHTVITAHRGLPTAELFSQLDKMKKGDIFYLHVLDQVLAYQVDQIVTVE
PNDFEPVLIQHGQDYATLLTCTPYMINSHRLLVRGKRIPYTAPIAERNRA
VRERGQFWLWLLLGAMAVILLLLYRVYRNRRIVKGLEKQLEGRHVKD

ORF8_{—}19FTW is a putative sortase. An example of an amino acid sequence of ORF8_{—}19FTW is set forth in SEQ ID NO:228.

SEQ ID NO: 228

MSRTKLRALLGYLLMLVACLIPIYCFGQMVLQSLGQVKGHATFVKSMTTE
MYQEQQNHSLAYNQRLASQNRIVDPFLAEGYEVNYQVSDDPDAVYGYLSI
PSLEIMEPVYLGADYHHLGMGLAHVDGTPLPLDGTGIRSVIAGHRAEPSH
VFFRHLDQLKVGDALYYNDGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFLGILFVLWKLARLLRGK

As discussed above, a S. pneumoniae AI sequence is present in the 23F Taiwan 15 S. pneumoniae genome. Examples of S. pneumoniae AI sequences from 23F Taiwan 15 are set forth below.

ORF2_{—}23FTW is a transcriptional regulator. An example of an amino acid sequence of ORF2_{—}23FTW is set forth in SEQ ID NO:229.

SEQ ID NO: 229

MLNKYIEKRITDKITILNILLDIRSIELDELSTLTSLQSKSLLSILQELQ
ETFEEELTFNLDTQQVQLIEHHSHQTNYYFQHLYNQSTILKILRFFLLQG
NQSFNEFTQKEYISIATGYRVRQKCGLLLRSVGLDLVKNQVVGPEYRIRF
LIALLQFHFGIEIYDLNGDSMDWVTHMIVQSNSQLSHELLEITPDEYVHF
SILVALTWKRREFPLEFPESKEFEKLKNLFMYPILMEHCQTYLEPHANMT
FTQEELDYIFLVYCSANSSFSKDKWNQEKKTHTIQLILQHTRGKHLLSKF
KNILGNDISNSLSFLTALTFLTRTFLFGLQNLVPYYNYYEHYGIESDKPL
YHISKAIVQEWMTEQKIEGVIDQHRLYLFSLYLTETIFSSLPAIPIFIIL
NNQADVNLIKSIILRNFTDKVASVTGYNILISPPPSEEHLTEPLIIITTK
EYLPYVKKQYPKGKHHFLTIALDLHVSQQRLIYQTIVDIRKEAFDKRVAM
IAKKAHYLL

ORF3_{—}23FTW is a cell wall surface protein. An example of an amino acid sequence of ORF3_{—}23FTW is set forth in SEQ ID NO:230.

SEQ ID NO: 230

MKKVRKIFQKAVAGLCCISQLTAFSSIVALAETPETSPAIGKVVIKETGE
GGALLGDAVFELKNNTDGTTVSQRTEAQTGEAIFSNIKPGTYTLTEAQPP
VGYKPSTKQWTVEVEKNGRTTVQGEQVENREEALSDQYPQTGTYPDVQTP
YQIIKVDGSEKNGQHKALNPNPYERVIPEGTLSKRIYQVNNLDDNQYGIE
LTVSGKTVYEQKDKSVPLDVVILLDNSNSMSNIRNKNARRAERAGEATRS
LIDKITSDPENRVALVTYASTIFDGTEFTVEKGVADKNGKRLNDSLFWNY
DQTSFTTNTKDYSYLKLTNDKNDIVELKNKVPTEAEDHDGNRLMYQFGAT
FTQKALMKADEILTQQARQNSQKVIFHITDGVPTMSYPINFNHATFAPSY
QNQLNAFFSKSPNKDGILLSDFITQATSGEHTIVRGSGQSYQMFTDKTVY
EKGAPAAFPVKPEKYSEMKAAGYAVIGDPINGGYIWLNWRESILAYPFNS
NTAKITNHGDPTRWYYNGNIAPDGYDVFTVGIGINGDPGTDEATATSFMQ
SISSKPENYTNVTDTTKILEQLNRYFHTIVTEKKSIENGTITDPMGELID
LQLGTDGRFDPADYTLTANDGSRLENGQAVGGPQNDGGLLKNAKVLYDTT
EKRIRVTGLYLGTDEKVTLTYNVRLNDEFVSNKFYDTNGRTTLHPKEVEQ
NTVRDFPIPKIRDVRKYPEITISKEKKLGDIEFIKVNKNDKKPLRDAVFS
LQKQHPDYPDIYGAIDQNGTYQNVRTGEDGKLTFKNLSDGKYRLFENSEP
AGYKPVQNKPIVAFQIVNGEVRDVTSIVPQDIPAGYEFTNDKHYITNEPI
PPKREYPRTGGIGMLPFYLIGCMMMGGVLLYTRKHP

ORF4_{—}23FTW is a cell wall surface protein. An example of an amino acid sequence of ORF4_{—}23FTW is set forth in SEQ ID NO:231.

SEQ ID NO: 231

MKSINKFLTILAALLLTVSSLFSAATVFAAEQKTKTLTVHKLLMTDQELD
AWNSDAITTAGYDGSQNFEQFKQLQGVPQGVTEISGVAFELQSYTGPQGK
EQENLTNDAVWTAVNKGVTTETGVKFDTEVLQGTYRLVEVRKESTYVGPN
GKVLTGMKAVPALITLPLVNQNGVVENAHVYPKNSEDKPTATKTFDTAAG
FVDPGEKGLAIGTKVPYIVTTTIPKNSTLATAFWSDEMTEGLDYNGDVVV
NYNGQPLDNSHYTLEAGHNGFILKLNEKGLEAINGKDAEATITLKYTATL
NALAVADVPEANDVTFHYGNNPGHGNTPKPNKPKNGELTITKTWADAKDA
PIAGVEVTFDLVNAQTGEVVKVPGHETGIVLNQTNNWTFTATGLDNNTEY
KFVERTIKGYSADYQTITETGKIAVKNWKDENPEPINPEEPRVKTYGKKF
VKVDQKDERLKEAQFVVKNWQGKYLALKSAAQQAVNEKAAAEAKQALDAA
IAAYTNAADKNAAQAVVDAAQKTYNDNYRAARFGYVEVERKEDALVLTSN
TDGQFQISGLAAGSYTLEETKAPEGFAKLGDVKFEVGAGSWNQGDFNYLK
DVQKNDATKVVNKKITIPQTGGIGTIIFAVAGAVIMGIAVYAYVKNNKDE
DQLA

ORF5_{—}23FTW is a cell wall surface protein. An example of an amino acid sequence of ORF5_{—}23FTW is set forth in SEQ ID NO:232.

SEQ ID NO: 232

MTMQKMQKMISRIFFVMALVFSLVWGAHAVQAQEDHTLVLQLENYQEVVS
QLPSRDGHRLQVWKLDDSYSYDNRVQIVRDLHSWDENKLSSFKKTSFEMT
FLENQIEVSHIPNGLYYVRSIIQTDAVSYPAEFLFEMTDQTVEPLVIVAK
KADTVTTKVKLIKVDQDHNRLEGVGFKLVSVARDGSEKEVPLIGEYRYSS
SGQVGRTLYTDKNGEIVVTNLPLGTYRFKEVEPLAGYTVTTMDTDVQLVD
HQLVTITVVNQKLPRGNVDFMKVDGRTNTSLQGAMFKVMKEENGHYTPVL
QNGKEVVVASGKDGRFRVEGLEYGTYYLWELQAPTGYVQLTSPVSFTIGK
DTRKELVTVVKNNKRPRIDVPDTGEETLYILMLVAILLFGSGYYLTKKTN
N

ORF6_{—}23FTW is a putative sortase. An example of an amino acid sequence of ORF6_{—}23FTW is set forth in SEQ ID NO:233.

SEQ ID NO: 233

MLIKMVKTKKQKRNNLLLGVVFFIGMAVMAYPLVSRLYYRVESNQQIADF
DKEKATLDEADIDERMKLAQAFNDSLNNVVSGDPWSEEMKKKGRAEYARM
LEIHERMGHVEIPVIDVDLPVYAGTAEEVLQQGAGQLEGTSLPIGGNSTH
AVITAHTGLPTAKMFTDLTKLKVGDKFYVHNIKEVMAYQVDQVKVIEPTN
FDDLLIVPGHDYVTLLTCTPYMINTHRLLVRGHRIPYVAEVEEEFIAANK
LSHLYRYLFYVAVGLIVILLWIIRRLRKKKKQPEKALKALKAARKEVKVE
DGQQ

ORF7_{—}23FTW is a putative sortase. An example of an amino acid sequence of ORF7_{—}23FTW is set forth in SEQ ID NO:234.

SEQ ID NO: 234

MDNSRRSRKKGTKKKKHPLILLLIFLVGFAVAIYPLVSRYYYRIESNEVI
KEFDETVSQMDKAELEERWRLAQAFNATLKPSEILDPFTEQEKKKGCSEY
ANMLKVHERIGYVEIPAIDQEIPMYVGTSEEILQKGAGLLEGASLPVGGE
NTHTVVTAHRGLPTAELFSQLDKMKKGDVFYLHVLDQVLAYQVDQILTVE
PNDFEPVLIQHGKDYATLLTCTPYMINSHRLLVRGKRIPYTAPIAERNRA
VRERGQFWLWLLLAALVMILVLSYGVYRHRRIVKGLEKQLEEHHVKG

ORF8_{—}23FTW is a putative sortase. An example of an amino acid sequence of ORF8_{—}23FTW is set forth in SEQ ID NO:235.

SEQ ID NO: 235

MSKAKLQKLLGYLLMLVALVIPVYCFGQMVLQSLGQVKGHEIFSESVTAD
SYQEQLQRSLDYNQRLDSQNRIVDPFLAEGYEVNYQVSDDPDAVYGYLSI
PSLEIMEPVYLGADYHHLAMGLAHVDGTPLPVEGKGIRSVIAGHRAEPSH
VFFRHLDQLKVGDALYYDNGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFLGILFVLWKLARLLRGK

As discussed above, a S. pneumoniae AI sequence is present in the 23F Poland 16 S. pneumoniae genome. Examples of S. pneumoniae AI sequences from 23F Poland 16 are set forth below.

ORF2_{—}23FP is a transcriptional regulator. An example of an amino acid sequence of ORF2_{—}23FP is set forth in SEQ ID NO:236.

SEQ ID NO: 236

MLNKYIEKRITDKITILNILLDIRSIELDELSTLTSLQSKSLLSILQELQ
ETFEEELTFNLDTQQVQLIEHHSHQTNYYFHQLYNQSTILKILRFFLLQG
NQSFNEFTQKEYISIATGYRVRQKCGLLLRSVGLDLVKNQVVGPEYRIRF
LIALLQFHFGIEIYDLNDGSMDWVTHMIVQSNSQLSHELLEITPDEYVHF
SILVALTWKRREFPLEFPESKEFEKLKNLFMYPILMEHCQTYLEPHANMT
FTQEELDYIFLVYCSANSSFSKDKWNQEKKTHTIQLILQHTRGKHLLSKF
NKNILGNDISNSLSFLTALTFLTRTFLFGLQNLVPYYNYYEHGIESDKPL
YHISKAIVQEWMTEQKIEGVIDQHRLYLFSLYLTETIFSSLPAIPIFIIL
NNQADVNLIKSIILRNFTDKVASVTGYNILISPPPSEEHLTEPLIIITTK
EYLPYVKKQYPKGKHHFLTIALDLHVSQQRLIYQTIVDIRKEAFDKRVAM
IAKKAHYLL

ORF3_{—}23FP is a cell wall surface protein. An example of an amino acid sequence of ORF3_{—}23FP is set forth in SEQ ID NO:237.

SEQ ID NO: 237

MKKVRKIFQKAVAGLCCISQLTAFSSIVALAETPETSPAIGKVVIKETGE
GGALLGDAVFELKNNTDGTTVSQRTEAQTGEAIFSNIKPGTYTLTEAQPP
VGYKPSTKQWTVEVEKNGRTTVQGEQVENREEALSDQYPQTGTYPDVQTP
YQIIKVDGSEKNGQHKALNPNPYERVIPEGTLSKRIYQVNNLDDNQYGIE
LTVSGKTTVETKEASTPLDVVILLDNSNSMSNIRHNHAHRAEKAGEATRA
LVDKITSNPDNRVALVTYGSTIFDGSEATVEKGVADANGKILNDSALWTF
DRTTFTAKTYNYSFLNLTSDPTDQITIKDRIPSDAEELNKDKLMYQFGAT
FTQKALMTADDILTKQARPNSKKVIFHITDGVPTMSYPINFKYTFTTQSY
RTQLNNFKAKTPNSSGILLEDFVTWSADGEHKIVRGDGESYQMFTKKPVT
DQYGVHQILSITSMEQRAKLVSAGYRFYGTDLYLYWRDSILAYPFNSSTD
WITNHGDPTTWYYNGNMAQDGYDVFTVGVGVNGDPGTDEATATRFMQSIS
SSPDNYTNVADPSQILQELNRYFYTIVNEKKSIENGTITDPMGELIDFQL
GADGRFDPADYTLTANDGSSLVNNVPTGGPQNDGGLLKNAKVFYDTTEKR
IRVTGLYLGTGEKVTLTYNVRLNDQFVSNKFYDTNGRTTLHPKEVEKNTV
RDFPIPKIRDVRKYPEITIPKEKKLGEIEFIKINKNDKKPLRDAVFSLQK
QHPDYPDIYGAIDQNGTYQNVRTGEDGKLTFKNLSDGKYRLFENSEPAGY
KPVQNKPIVAFQIVNGEVRDVTSIVPQDIPAGYEFTNDKHYITNEPIPPK
REYPRTGGIGMLPFYLIGCMMMGGVLLYTRKNP

ORF4_{—}23FP is a cell wall surface protein. An example of an amino acid sequence of ORF4_{—}23FP is set forth in SEQ ID NO:238.

SEQ ID NO: 238

MKSINKFLTMLAALLLTASSLFSAATVFAADNVSTAPDAVTKTLTIHKLL
LSEDDLKTWDTNGPKGYDGTQSSLKDLTGVVAEEIPNVYFELQKYNLTDG
KEKENLKDDSKWTTVHGGLTTKDGLKIETSTLKGVYRIREDRTKTTYVGP
NGQVLTGSKAVPALVTLPLVNNNGTVIDAHVFPKNSYNKPVVDKRIADTL
NYNDQNGLSIGTKIPYVVNTTIPSNATFATSFWSDEMTEGLTYNEDVTIT
LNNVAMDQADYEVTKGINGFNLKLTEAGLAKINGKDADQKIQITYSATLN
SLAVADIPSENDITYHYGNHQDHGNTPKPTKPNNGQITVTKTWDSQPAPE
GCKATVQLVNAKTGEKVGAPVELSENNWTYTWSGLDNSIEYKVEEEYNGY
SAEYTVESKGKLGVKNWKDNNPAPINLEEPRVKTYGKKFVKVDQKDTRLE
NAQFVVKKADSNKYIAFKSTAQQAADEKAAATAKQKLDAAVAAYTNAADK
QAAQALVDQAQQEYNVAYKEAKFGYVEVAGKDEAMVLTSNTDGQFQISGL
AAGTYKLEEIKAPEGFAKIDDVEFVVGAGSWNQGEFNYLKDVQKNDATKV
VNKKITIPQTGGIGTIIFAVAGAVIMGIAVYAYVKNNKDEDQLA

ORF5_{—}23FP is a cell wall surface protein. An example of an amino acid sequence of ORF5_{—}23FP is set forth in SEQ ID NO:239.

SEQ ID NO: 239

MTMQKMQKMISRIFFVMALVFSLVWGAHAVQAQEDHTLVLQLENYQEVVS
QLPSRDGHRLQVWKLDDSYSYDNRVQIVRDLHSWDENKLSSFKKTSFEMT
FLENQIEVSHIPNGLYYVRSIIQTDAVSYPAEFLFEMTDQTVEPLVIVAK
KADTVTTKVKLIKVDQDHNRLEGVGFKLVSVARDGSEKEVPLIGEYRYSS
SGQVGRTLYTDKNGEIVVTNLPLGTYRFKEVEPLAGYAVTTMDTDVQLVD
HQLVTITVVNQKLPRGNVDFMKVDGRTNTSLQGAMFKVMKEENGHYTPVL
QNGKEVVVASGKDGRFRVEGLEYGTYYLWELQAPTGYVQLTSPVSFTIGK
DTRKELVTVVKNNKRPRIDVPDTGEETLYILMLVAILLFGSGYYLTKKTN
N

ORF6_{—}23FP is a putative sortase. An example of an amino acid sequence of ORF6_{—}23FP is set forth in SEQ ID NO:240.

SEQ ID NO: 240

MLIKMAKTKKQKRNNLLLGVVFFIGIAVMAYPLVSRLYYRVESNQQIADF
DKEKATLDEADIDERMKLAQAFNDSLNNVVSGDPWSEEMKKKGRAEYARM
LEIHERMGHVEIPAIDVDLPVYAGTAEEVLQQGAGHLEGTSLPIGGNSTH
AVITAHTGLPTAKMFTDLTKLKVGDKFYVHNIKEVMAYQVDQVKVEIPTN
FDDLLIVPGHDYVTLLTCTPYMINTHRLLVRGHRIPYVAEVEEEFIAANK
LSHLYRYLFYVAVGLIVILLWIIRRLRKKKRQSERALKALKEATKEVKVE
DE

ORF7_{—}23FP is a putative sortase. An example of an amino acid sequence of ORF7_{—}23FP is set forth in SEQ ID NO:241.

SEQ ID NO: 241

MSKSRYSRKKSVKKKKNPFILLLIFLVGLAVAMYPLVSRYYYRIESNEVI
KEFDETVSQMDKAELEERWRLAQAFNATLKPSEILDPFTEQEKKKGVSEY
ANMLKVHERIGYVEIPAIDQEIPMYVGTSEEILQKGAGLLEGASLPVGGE
NTHTVVTAHRGLPTAELFWQLDKMKKGDIFYLHVLDQVLAYQVDQIVTVE
PNDFEPVLIQHGEDYATLLTCTPYMINSHRLLVRGKRIPYTAPIAERNRA
VRERGQFWLWLLLGAMAVILLLLYRVYRNRRIVKGLEKQLEGHHVKD

ORF8_{—}23FP is a putative sortase. An example of an amino acid sequence of ORF8_{—}23FP is set forth in SEQ ID NO:242.

SEQ ID NO: 242

MSRTKLRALLGYLLMLVACLIPIYCFGQMVLQSLGQVKGHATFVKSMTTE
MYQEQQNHSLAYNQRLASQNRIVDPFLAEGYEVNYQVSDDPDAVYGYLSI
PSLEIMEPVYLGADYHHLGMGLAHVDGTPLPLDGTGIRSVIAGHRAEPSH
VFFRHLDQLKVGDALYYNDGQEIVEYQMMDTEIILPSEWEKLESVSSKNI
MTLITCDPIPTFNKRLLVNFERVAVYQKSDPQTAAVARVAFTKEGQSVSR
VATSQWLYRGLVVLAFLGILFVLWKLARLLRGK

Immunogenic compositions of the invention comprising AI antigens may further comprise one or more antigenic agents. Preferred antigens include those listed below. Additionally, the compositions of the present invention may be used to treat or prevent infections caused by any of the below-listed microbes. Antigens for use in the immunogenic compositions include, but are not limited to, one or more of the following set forth below, or antigens derived from one or more of the following set forth below:

Bacterial Antigens

N. meningitides: a protein antigen from N. meningitides serogroup A, C, W135, Y, and/or B (1-7); an outer-membrane vesicle (OMV) preparation from N. meningitides serogroup B. (8, 9, 10, 11); a saccharide antigen, including LPS, from N. meningitides serogroup A, B, C W135 and/or Y, such as the oligosaccharide from serogroup C (see PCT/US99/09346; PCT IB98/01665; and PCT IB99/00103);

Streptococcus pneumoniae: a saccharide or protein antigen, particularly a saccharide from Streptooccus pneumoniae;

Streptococcus agalactiae: particularly, Group B streptococcus antigens;

Streptococcus pyogenes: particularly, Group A streptococcus antigens;

Enterococcus faecalis or Enterococcus faecium: Particularly a trisaccharide repeat or other Enterococcus derived antigens provided in U.S. Pat. No. 6,756,361;

Helicobacter pylori: including: Cag, Vac, Nap, HopX, HopY and/or urease antigen;

Bordetella pertussis: such as petussis holotoxin (PT) and filamentous haemagglutinin (FHA) from B. pertussis, optionally also combination with pertactin and/or agglutinogens 2 and 3 antigen;

Staphylococcus aureus: including S. aureus type 5 and 8 capsular polysaccharides optionally conjugated to nontoxic recombinant Pseudomonas aeruginosa exotoxin A, such as StaphVAX™, or antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin);

Staphylococcus epidermis: particularly, S. epidermidis slime-associated antigen (SAA);

Staphylococcus saprophyticus: (causing urinary tract infections) particularly the 160 kDa hemagglutinin of S. saprophyticus antigen;

Pseudomonas aeruginosa: particularly, endotoxin A, Wzz protein, P. aeruginosa LPS, more particularly LPS isolated from PAO1 (O5 serotype), and/or Outer Membrane Proteins, including Outer Membrane Proteins F (OprF) (Infect Immun. 2001 May; 69(5): 3510-3515);

Bacillus anthracis (anthrax): such as B. anthracis antigens (optionally detoxified) from A-components (lethal factor (LF) and edema factor (EF)), both of which can share a common B-component known as protective antigen (PA);

Moraxella catarrhalis: (respiratory) including outer membrane protein antigens (HMW-OMP), C-antigen, and/or LPS;

Yersinia pestis (plague): such as F1 capsular antigen (Infect Immun. 2003 January; 71(1)): 374-383, LPS (Infect Immun. 1999 October; 67(10): 5395), Yersinia pestis V antigen (Infect Immun. 1997 November; 65(11): 4476-4482);

Yersinia enterocolitica (gastrointestinal pathogen): particularly LPS (Infect Immun. 2002 August; 70(8): 4414);

Yersinia pseudotuberculosis: gastrointestinal pathogen antigens;

Mycobacterium tuberculosis: such as lipoproteins, LPS, BCG antigens, a fusion protein of antigen 85B (Ag85B) and/or ESAT-6 optionally formulated in cationic lipid vesicles (Infect Immun. 2004 October; 72(10): 6148), Mycobacterium tuberculosis (Mtb) isocitrate dehydrogenase associated antigens (Proc Natl Acad Sci USA. 2004 Aug. 24; 101(34): 12652), and/or MPT51 antigens (Infect Immun. 2004 July; 72(7): 3829);

Legionella pneumophila (Legionnairs' Disease): L. pneumophila antigens—optionally derived from cell lines with disrupted asd genes (Infect Immun. 1998 May; 66(5): 1898);

Rickettsia: including outer membrane proteins, including the outer membrane protein A and/or B (OmpB) (Biochim Biophys Acta. 2004 Nov. 1; 1702(2):145), LPS, and surface protein antigen (SPA) (J Autoimmun. 1989 June; 2 Suppl:81);

E. coli: including antigens from enterotoxigenic E. coli (ETEC), enteroaggregative E. coli (EAggEC), diffusely adhering E. coli (DAEC), enteropathogenic E. coli (EPEC), and/or enterohemorrhagic E. coli (EHEC);

Vibrio cholerae: including proteinase antigens, LPS, particularly lipopolysaccharides of Vibrio cholerae II, O1 Inaba O-specific polysaccharides, V. cholera O139, antigens of IEM108 vaccine (Infect Immun. 2003 October; 71(10):5498-504), and/or Zonula occludens toxin (Zot);

Salmonella typhi (typhoid fever): including capsular polysaccharides preferably conjugates (Vi, i.e. vax-TyVi);

Salmonella typhimurium (gastroenteritis): antigens derived therefrom are contemplated for microbial and cancer therapies, including angiogenesis inhibition and modulation of flk;

Listeria monocytogenes (sytemic infections in immunocompromised or elderly people, infections of fetus): antigens derived from L. monocytogenes are preferably used as carriers/vectors for intracytoplasmic delivery of conjugates/associated compositions of the present invention;

Porphyromonas gingivalis: particularly, P. gingivalis outer membrane protein (OMP);

Tetanus: such as tetanus toxoid (TT) antigens, preferably used as a carrier protein in conjunction/conjugated with the compositions of the present invention;

Diphtheria: such as a diphtheria toxoid, preferably CRM₁₉₇, additionally antigens capable of modulating, inhibiting or associated with ADP ribosylation are contemplated for combination/co-administration/conjugation with the compositions of the present invention, the diphtheria toxoids are preferably used as carrier proteins;

Borrelia burgdorferi (Lyme disease): such as antigens associated with P39 and P13 (an integral membrane protein, Infect Immun. 2001 May; 69(5): 3323-3334), VlsE Antigenic Variation Protein (J Clin Microbiol. 1999 December; 37(12): 3997);

Haemophilus influenzae B: such as a saccharide antigen therefrom;

Klebsiella: such as an OMP, including OMP A, or a polysaccharide optionally conjugated to tetanus toxoid;

Neiserria gonorrhoeae: including, a Por (or porn) protein, such as PorB (see Zhu et al., Vaccine (2004) 22:660-669), a transferring binding protein, such as TbpA and TbpB (See Price et al., Infection and Immunity (2004) 71(1):277-283), a opacity protein (such as Opa), a reduction-modifiable protein (Rmp), and outer membrane vesicle (OMV) preparations (see Plante et al., J Infectious Disease (2000) 182:848-855), also see e.g. WO99/24578, WO99/36544, WO99/57280, WO02/079243);

Chlamydia pneumoniae: particularly C. pneumoniae protein antigens;

Chlamydia trachomatis: including antigens derived from serotypes A, B, Ba and C are (agents of trachoma, a cause of blindness), serotypes L₁, L₂ & L₃ (associated with Lymphogranuloma venereum), and serotypes, D-K;

Treponema pallidum (Syphilis): particularly a TmpA antigen; and

Haemophilus ducreyi (causing chancroid): including outer membrane protein (DsrA).

Where not specifically referenced, further bacterial antigens of the invention may be capsular antigens, polysaccharide antigens or protein antigens of any of the above. Further bacterial antigens may also include an outer membrane vesicle (OMV) preparation. Additionally, antigens include live, attenuated, split, and/or purified versions of any of the aforementioned bacteria. The bacterial or microbial derived antigens of the present invention may be gram-negative or gram-positive and aerobic or anaerobic.

Additionally, any of the above bacterial-derived saccharides (polysaccharides, LPS, LOS or oligosaccharides) can be conjugated to another agent or antigen, such as a carrier protein (for example CRM₁₉₇). Such conjugation may be direct conjugation effected by reductive amination of carbonyl moieties on the saccharide to amino groups on the protein, as provided in U.S. Pat. No. 5,360,897 and Can J Biochem Cell Biol. 1984 May; 62(5):270-5. Alternatively, the saccharides can be conjugated through a linker, such as, with succinamide or other linkages provided in Bioconjugate Techniques, 1996 and CRC, Chemistry of Protein Conjugation and Cross-Linking, 1993.

Viral Antigens

Influenza: including whole viral particles (attenuated), split, or subunit comprising hemagglutinin (HA) and/or neuraminidase (NA) surface proteins, the influenza antigens may be derived from chicken embryos or propogated on cell culture, and/or the influenza antigens are derived from influenza type A, B, and/or C, among others;

Respiratory syncytial virus (RSV): including the F protein of the A2 strain of RSV (J Gen Virol. 2004 November; 85(Pt 11):3229) and/or G glycoprotein;

Parainfluenza virus (PIV): including PIV type 1, 2, and 3, preferably containing hemagglutinin, neuraminidase and/or fusion glycoproteins;

Poliovirus: including antigens from a family of picornaviridae, preferably poliovirus antigens such as OPV or, preferably IPV;

Measles: including split measles virus (MV) antigen optionally combined with the Protollin and or antigens present in MMR vaccine;

Mumps: including antigens present in MMR vaccine;

Rubella: including antigens present in MMR vaccine as well as other antigens from Togaviridae, including dengue virus;

Rabies: such as lyophilized inactivated virus (RabAvert™);

Flaviviridae viruses: such as (and antigens derived therefrom) yelow fever virus, Japanese encephalitis virus, dengue virus (types 1, 2, 3, or 4), tick borne encephalitis virus, and West Nile virus;

Caliciviridae; antigens therefrom;

HIV: including HIV-1 or HIV-2 strain antigens, such as gag (p24gag and p55gag), env (gp160 and gp41), pol, tat, nef, rev vpu, miniproteins, (preferably p55 gag and gp140v delete) and antigens from the isolates HIV_{IIIb}, HIV_{SF2}, HIV_{LAV}, HIV_{LAI}, HIV_{MN}, HIV-1_{CM235}, HIV-1_{US4}, HIV-2; simiam immunodeficiency virus (SIV) among others;

Rotavirus: including VP4, VPS, VP6, VP7, VP8 proteins (Protein Expr Purif. 2004 December; 38(2):205) and/or NSP4;

Pestivirus: such as antigens from classical porcine fever virus, bovine viral diarrhoea virus, and/or border disease virus;

Parvovirus: such as parvovirus B19;

Coronavirus: including SARS virus antigens, particularly spike protein or proteases therefrom, as well as antigens included in WO 04/92360;

Hepatitis A virus: such as inactivated virus;

Hepatitis B virus: such as the surface and/or core antigens (sAg), as well as the presurface sequences, pre-S1 and pre-S2 (formerly called pre-S), as well as combinations of the above, such as sAg/pre-S1, sAg/pre-S2, sAg/pre-S1/pre-S2, and pre-S1/pre-S2, (see, e.g., AHBV Vaccines—Human Vaccines and Vaccination, pp. 159-176; and U.S. Pat. Nos. 4,722,840, 5,098,704, 5,324,513; Beames et al., J. Virol. (1995) 69:6833-6838, Birnbaum et al., J. Virol. (1990) 64:3319-3330; and Zhou et al., J. Virol. (1991) 65:5457-5464);

Hepatitis C virus: such as E1, E2, E1/E2 (see, Houghton et al., Hepatology (1991) 14:381), NS345 polyprotein, NS 345-core polyprotein, core, and/or peptides from the nonstructural regions (International Publication Nos. WO 89/04669; WO 90/11089; and WO 90/14436);

Delta hepatitis virus (HDV): antigens derived therefrom, particularly δ-antigen from HDV (see, e.g., U.S. Pat. No. 5,378,814);

Hepatitis E virus (HEV); antigens derived therefrom;

Hepatitis G virus (HGV); antigens derived therefrom;

Varcicella zoster virus: antigens derived from varicella zoster virus (VZV) (J. Gen. Virol. (1986) 67:1759);

Epstein-Barr virus: antigens derived from EBV (Baer et al., Nature (1984) 310:207);

Cytomegalovirus: CMV antigens, including gB and gH (Cytomegaloviruses (J. K. McDougall, ed., Springer-Verlag 1990) pp. 125-169);

Herpes simplex virus: including antigens from HSV-1 or HSV-2 strains and glycoproteins gB, gD and gH (McGeoch et al., J. Gen. Virol. (1988) 69:1531 and U.S. Pat. No. 5,171,568);

Human Herpes Virus: antigens derived from other human herpesviruses such as HHV6 and HHV7; and

HPV: including antigens associated with or derived from human papillomavirus (HPV), for example, one or more of E1-E7, L1, L2, and fusions thereof, particularly the compositions of the invention may include a virus-like particle (VLP) comprising the L1 major capsid protein, more particular still, the HPV antigens are protective against one or more of HPV serotypes 6, 11, 16 and/or 18.

Further provided are antigens, compositions, methods, and microbes included in Vaccines, 4^{th} Edition (Plotkin and Orenstein ed. 2004); Medical Microbiology 4^{th} Edition (Murray et al. ed. 2002); Virology, 3rd Edition (W. K. Joklik ed. 1988); Fundamental Virology, 2nd Edition (B. N. Fields and D. M. Knipe, eds. 1991), which are contemplated in conjunction with the compositions of the present invention.

Additionally, antigens include live, attenuated, split, and/or purified versions of any of the aforementioned viruses.

Fungal Antigens

Fungal antigens for use herein, associated with vaccines include those described in: U.S. Pat. Nos. 4,229,434 and 4,368,191 for prophylaxis and treatment of trichopytosis caused by Trichophyton mentagrophytes; U.S. Pat. Nos. 5,277,904 and 5,284,652 for a broad spectrum dermatophyte vaccine for the prophylaxis of dermatophyte infection in animals, such as guinea pigs, cats, rabbits, horses and lambs, these antigens comprises a suspension of killed T. equinum, T. mentagrophytes (var. granulare), M. canis and/or M. gypseum in an effective amount optionally combined with an adjuvant; U.S. Pat. Nos. 5,453,273 and 6,132,733 for a ringworm vaccine comprising an effective amount of a homogenized, formaldehyde-killed fungi, i.e., Microsporum canis culture in a carrier; U.S. Pat. No. 5,948,413 involving extracellular and intracellular proteins for pythiosis. Additional antigens identified within antifungal vaccines include Ringvac bovis LTF-130 and Bioveta.

Further, fungal antigens for use herein may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme.

Fungal pathogens for use as antigens or in derivation of antigens in conjunction with the compositions of the present invention comprise Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides Blastomyces dermatidis, Cryptococcus neoformans, Geotrichum clavatum, Histoplasma capsulatum, Klebsiella pneumoniae, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, and Saksenaea spp.

Other fungi from which antigens are derived include Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Processes for producing a fungal antigens are well known in the art (see U.S. Pat. No. 6,333,164). In a preferred method a solubilized fraction extracted and separated from an insoluble fraction obtainable from fungal cells of which cell wall has been substantially removed or at least partially removed, characterized in that the process comprises the steps of: obtaining living fungal cells; obtaining fungal cells of which cell wall has been substantially removed or at least partially removed; bursting the fungal cells of which cell wall has been substantially removed or at least partially removed; obtaining an insoluble fraction; and extracting and separating a solubilized fraction from the insoluble fraction.

STD Antigens

In particular embodiments, microbes (bacteria, viruses and/or fungi) against which the present compositions and methods can be implement include those that cause sexually transmitted diseases (STDs) and/or those that display on their surface an antigen that can be the target or antigen composition of the invention. In a preferred embodiment of the invention, compositions are combined with antigens derived from a viral or bacterial STD. Antigens derived from bacteria or viruses can be administered in conjunction with the compositions of the present invention to provide protection against at least one of the following STDs, among others: chlamydia, genital herpes, hepatitis (particularly HCV), genital warts, gonorrhoea, syphilis and/or chancroid (See, WO00/15255).

In another embodiment the compositions of the present invention are co-administered with an antigen for the prevention or treatment of an STD.

Antigens derived from the following viruses associated with STDs, which are described in greater detail above, are preferred for co-administration with the compositions of the present invention: hepatitis (particularly HCV), HPV, HIV, or HSV.

Additionally, antigens derived from the following bacteria associated with STDs, which are described in greater detail above, are preferred for co-administration with the compositions of the present invention: Neiserria gonorrhoeae, Chlamydia pneumoniae, Chlamydia trachomatis, Treponema pallidum, or Haemophilus ducreyi.

Respiratory Antigens

The antigen may be a respiratory antigen and could further be used in an immunogenic composition for methods of preventing and/or treating infection by a respiratory pathogen, including a virus, bacteria, or fungi such as respiratory syncytial virus (RSV), PIV, SARS virus, influenza, Bacillus anthracia, particularly by reducing or preventing infection and/or one or more symptoms of respiratory virus infection. A composition comprising an antigen described herein, such as one derived from a respiratory virus, bacteria or fungus is administered in conjunction with the compositions of the present invention to an individual which is at risk of being exposed to that particular respiratory microbe, has been exposed to a respiratory microbe or is infected with a respiratory virus, bacteria or fungus. The composition(s) of the present invention is/are preferably co-administered at the same time or in the same formulation with an antigen of the respiratory pathogen. Administration of the composition results in reduced incidence and/or severity of one or more symptoms of respiratory infection.

Pediatric/Geriatric Antigens

In one embodiment the compositions of the present invention are used in conjunction with an antigen for treatment of a pediatric population, as in a pediatric antigen. In a more particular embodiment the pediatric population is less than about 3 years old, or less than about 2 years, or less than about 1 years old. In another embodiment the pediatric antigen (in conjunction with the composition of the present invention) is administered multiple times over at least 1, 2, or 3 years.

In another embodiment the compositions of the present invention are used in conjunction with an antigen for treatment of a geriatric population, as in a geriatric antigen.

Other Antigens

Other antigens for use in conjunction with the compositions of the present include hospital acquired (nosocomial) associated antigens.

In another embodiment, parasitic antigens are contemplated in conjunction with the compositions of the present invention. Examples of parasitic antigens include those derived from organisms causing malaria and/or Lyme disease.

In another embodiment, the antigens in conjunction with the compositions of the present invention are associated with or effective against a mosquito born illness. In another embodiment, the antigens in conjunction with the compositions of the present invention are associated with or effective against encephalitis. In another embodiment the antigens in conjunction with the compositions of the present invention are associated with or effective against an infection of the nervous system.

In another embodiment, the antigens in conjunction with the compositions of the present invention are antigens transmissible through blood or body fluids.

Antigen Formulations

In other aspects of the invention, methods of producing microparticles having adsorbed antigens are provided. The methods comprise: (a) providing an emulsion by dispersing a mixture comprising (i) water, (ii) a detergent, (iii) an organic solvent, and (iv) a biodegradable polymer selected from the group consisting of a poly(α-hydroxy acid), a polyhydroxy butyric acid, a polycaprolactone, a polyorthoester, a polyanhydride, and a polycyanoacrylate. The polymer is typically present in the mixture at a concentration of about 1% to about 30% relative to the organic solvent, while the detergent is typically present in the mixture at a weight-to-weight detergent-to-polymer ratio of from about 0.00001:1 to about 0.1:1 (more typically about 0.0001:1 to about 0.1:1, about 0.001:1 to about 0.1:1, or about 0.005:1 to about 0.1:1); (b) removing the organic solvent from the emulsion; and (c) adsorbing an antigen on the surface of the microparticles. In certain embodiments, the biodegradable polymer is present at a concentration of about 3% to about 10% relative to the organic solvent.

Microparticles for use herein will be formed from materials that are sterilizable, non-toxic and biodegradable. Such materials include, without limitation, poly(α-hydroxy acid), polyhydroxybutyric acid, polycaprolactone, polyorthoester, polyanhydride, PACA, and polycyanoacrylate. Preferably, microparticles for use with the present invention are derived from a poly(α-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. The microparticles may be derived from any of various polymeric starting materials which have a variety of molecular weights and, in the case of the copolymers such as PLG, a variety of lactide:glycolide ratios, the selection of which will be largely a matter of choice, depending in part on the coadministered macromolecule. These parameters are discussed more fully below.

Further antigens may also include an outer membrane vesicle (OMV) preparation.

Additional formulation methods and antigens (especially tumor antigens) are provided in U.S. patent Ser. No. 09/581,772.

Antigen References

The following references include antigens useful in conjunction with the compositions of the present invention:
1 International patent application WO99/24578
2 International patent application WO99/36544.
3 International patent application WO99/57280.
4 International patent application WO00/22430.
5 Tettelin et al. (2000) Science 287:1809-1815.
6 International patent application WO96/29412.
7 Pizza et al. (2000) Science 287:1816-1820.
8 PCT WO 01/52885.
9Bjune et al. (1991) Lancet 338(8775).
10 Fuskasawa et al. (1999) Vaccine 17:2951-2958.
11 Rosenqist et al. (1998) Dev. Biol. Strand 92:323-333.
12 Constantino et al. (1992) Vaccine 10:691-698.
13 Constantino et al. (1999) Vaccine 17:1251-1263.
14 Watson (2000) Pediatr Infect Dis J 19:331-332.
15 Rubin (20000) Pediatr Clin North Am 47:269-285,v.
16 Jedrzejas (2001) Microbiol Mol Biol Rev 65:187-207.
17 International patent application filed on 3 Jul. 2001 claiming priority from GB-0016363.4;WO 02/02606; PCT IB/01/00166.
18 Kalman et al. (1999) Nature Genetics 21:385-389.
19 Read et al. (2000) Nucleic Acids Res 28:1397-406.
20 Shirai et al. (2000) J. Infect. Dis 181(Suppl 3):S524-S527.
21 International patent application WO99/27105.
22 International patent application WO00/27994.
23 International patent application WO00/37494.
24 International patent application WO99/28475.
25 Bell (2000) Pediatr Infect Dis J 19:1187-1188.
26 Iwarson (1995) APMIS 103:321-326.
27 Gerlich et al. (1990) Vaccine 8 Suppl:S63-68 & 79-80.
28 Hsu et al. (1999) Clin Liver Dis 3:901-915.
29 Gastofsson et al. (1996) N. Engl. J. Med. 334-:349-355.
30 Rappuoli et al. (1991) TIBTECH 9:232-238.
31 Vaccines (1988) eds. Plotkin & Mortimer. ISBN 0-7216-1946-0.
32 Del Guidice et al. (1998) Molecular Aspects of Medicine 19:1-70.
33 International patent application WO93/018150.
34 International patent application WO99/53310.
35 International patent application WO98/04702.
36 Ross et al. (2001) Vaccine 19:135-142.
37 Sutter et al. (2000) Pediatr Clin North Am 47:287-308.
38 Zimmerman & Spann (1999) Am Fan Physician 59:113-118, 125-126.
39 Dreensen (1997) Vaccine 15 Suppl“52-6.
40 MMWR Morb Mortal Wkly rep 1998 Jan. 16: 47(1):12, 9.
41 McMichael (2000) Vaccine19 Suppl 1:S101-107.
42 Schuchat (1999) Lancer 353(9146):51-6.
43 GB patent applications 0026333.5, 0028727.6 & 0105640.7.
44 Dale (1999) Infect Disclin North Am 13:227-43, viii.
45 Ferretti et al. (2001) PNAS USA 98: 4658-4663.
46 Kuroda et al. (2001) Lancet 357(9264):1225-1240; see also pages 1218-1219.
47 Ramsay et al. (2001) Lancet 357(9251):195-196.
48 Lindberg (1999) Vaccine 17 Suppl 2:S28-36.
49 Buttery & Moxon (2000) J R Coil Physicians Long 34:163-168.
50 Ahmad & Chapnick (1999) Infect Dis Clin North Am 13:113-133, vii.
51 Goldblatt (1998) J. Med. Microbiol. 47:663-567.
52 European patent 0 477 508.
53 U.S. Pat. No. 5,306,492.
54 International patent application WO98/42721.
55 Conjugate Vaccines (eds. Cruse et al.) ISBN 3805549326, particularly vol. 10:48-114.
56 Hermanson (1996) Bioconjugate Techniques ISBN: 012323368 & 012342335X.
57 European patent application 0372501.
58 European patent application 0378881.
59 European patent application 0427347.
60 International patent application WO93/17712.
61 International patent application WO98/58668.
62 European patent application 0471177.
63 International patent application WO00/56360.
64 International patent application WO00/67161.

The contents of all of the above cited patents, patent applications and journal articles are incorporated by reference as if set forth fully herein.

There may be an upper limit to the number of Gram positive bacterial proteins which will be in the compositions of the invention. Preferably, the number of Gram positive bacterial proteins in a composition of the invention is less than 20, less than 19, less than 18, less than 17, less than 16, less than 15, less than 14, less than 13, less than 12, less than 11, less than 10, less than 9, less than 8, less than 7, less than 6, less than 5, less than 4, or less than 3. Still more preferably, the number of Gram positive bacterial proteins in a composition of the invention is less than 6, less than 5, or less than 4. Still more preferably, the number of Gram positive bacterial proteins in a composition of the invention is 3.

The Gram positive bacterial proteins and polynucleotides used in the invention are preferably isolated, i.e., separate and discrete, from the whole organism with which the molecule is found in nature or, when the polynucleotide or polypeptide is not found in nature, is sufficiently free of other biological macromolecules so that the polynucleotide or polypeptide can be used for its intended purpose.

### Fusion Proteins: GBS AI Sequences

The GBS AI proteins used in the invention may be present in the composition as individual separate polypeptides, but it is preferred that at least two (i.e. 2, 3, 4, 5, 6, 7, 8, 9, 10, 11, 12, 13, 14, 15, 16, 17, or 18) of the antigens are expressed as a single polypeptide chain (a “hybrid” or “fusion” polypeptide). Such fusion polypeptides offer two principal advantages: first, a polypeptide that may be unstable or poorly expressed on its own can be assisted by adding a suitable fusion partner that overcomes the problem; second, commercial manufacture is simplified as only one expression and purification need be employed in order to produce two polypeptides which are both antigenically useful.

The fusion polypeptide may comprise one or more AI polypeptide sequences. Preferably, the fusion comprises an AI surface protein sequence. Preferably, the fusion polypeptide includes one or more of GBS 80, GBS 104, and GBS 67. Most preferably, the fusion peptide includes a polypeptide sequence from GBS 80. Accordingly, the invention includes a fusion peptide comprising a first amino acid sequence and a second amino acid sequence, wherein said first and second amino acid sequences are selected from a GBS AI surface protein or a fragment thereof. Preferably, the first and second amino acid sequences in the fusion polypeptide comprise different epitopes.

Hybrids (or fusions) consisting of amino acid sequences from two, three, four, five, six, seven, eight, nine, or ten GBS antigens are preferred. In particular, hybrids consisting of amino acid sequences from two, three, four, or five GBS antigens are preferred.

Different hybrid polypeptides may be mixed together in a single formulation. Within such combinations, a GBS antigen may be present in more than one hybrid polypeptide and/or as a non-hybrid polypeptide. It is preferred, however, that an antigen is present either as a hybrid or as a non-hybrid, but not as both.

Hybrid polypeptides can be represented by the formula NH₂-A-{-X-L-}ₙ-B—COOH, wherein: X is an amino acid sequence of a GBS AI protein or a fragment thereof; L is an optional linker amino acid sequence; A is an optional N-terminal amino acid sequence; B is an optional C-terminal amino acid sequence; and n is 2, 3, 4, 5, 6, 7, 8, 9, 10, 11, 12, 13, 14 or 15.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {—X-L-}, linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁-X₂-L₂-COOH, etc. Linker amino acid sequence(s) -L- will typically be short (e.g. 20 or fewer amino acids i.e. 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. A useful linker is GSGGGG, with the Gly-Ser dipeptide being formed from a BamHI restriction site, thus aiding cloning and manipulation, and the (Gly)₄ tetrapeptide being a typical poly-glycine linker.

-A- is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art.

Most preferably, n is 2 or 3.

In some embodiment the GBS hybrid proteins of the invention may comprise first —X— moiety (—Xₐ—) and a second —X— moiety (—X_{b}—). The —Xₐ— moiety has one of the following amino acid sequences: SEQ ID NO:16, SEQ ID NO:126, SEQ ID NO:2, SEQ ID NO:11, SEQ ID NO:21, SEQ ID NO:27, SEQ ID NO:33, SEQ ID NO:35, SEQ ID NO:36.

The —X_{b}— moiety is related to —Xₐ— such that: (i) —X_{b}— has sequence identity to —Xₐ—, and/or (j) —X_{b}— comprises a fragment of —Xₐ—. Examples of this second type of hybrid protein include proteins in which two or more —X— moieties are identical, or in which they are variants of the same protein e.g. two polymorphic forms may be expressed as —Xₐ—X_{b}—, and three polymorphic forms may be expressed as —Xₐ—X_{b}—X_{c}— etc. The —Xa- and —Xb- moieties may be in either order from N-terminus to C-terminus.

The degree of ‘sequence identity’ referred to in (i) is preferably greater than 50% (ea. 60%, 70%, 80%, 90%, 95%, 99% or more, up to 100%). This includes mutants, homologs, orthologs, allelic variants etc. Identity is preferably determined by the Smith-Waterman homology search algorithm as implemented in the MPSRCH program (Oxford Molecular), using an 30 affine gap search with parameters gap open penalty=12 and gap extension penalty=1;. Typically, 50% identity or more between two proteins is considered as an indication of functional equivalence.

The ‘fragment’ referred to in (j) should consist of least m consecutive amino acids from an amino acid sequence from SEQ ID NO:16, SEQ ID NO:126, SEQ ID NO:2, SEQ ID NO:11, SEQ ID NO:21, SEQ ID NO:27, SEQ ID NO:33, SEQ ID NO:35, SEQ ID NO:36. and, depending on the particular sequence, m is 7 or more (ea. 8, 10,&rsqb;2, 14, 16, 18, 20, 25, 30, 35, 40, 50, 60, 70, 80, 90, 100, 150, 200 or more). Preferably the fragment comprises an epitope from an amino acid sequence from SEQ ID NO:16, SEQ ID NO:126, SEQ ID NO:2, SEQ ID NO:11, SEQ ID NO:21, SEQ ID NO:27, SEQ ID NO:33, SEQ ID NO:35, SEQ ID NO:36.

### Fusion Proteins: Gram Positive Bacteria AI Sequences

The Gram positive bacteria AI proteins used in the invention may be present in the composition as individual separate polypeptides, but it is preferred that at least two (i.e. 2, 3, 4, 5, 6, 7, 8, 9, 10, 11, 12, 13, 14, 15, 16, 17, or 18) of the antigens are expressed as a single polypeptide chain (a “hybrid” or “fusion” polypeptide). Such fusion polypeptides offer two principal advantages: first, a polypeptide that may be unstable or poorly expressed on its own can be assisted by adding a suitable fusion partner that overcomes the problem; second, commercial manufacture is simplified as only one expression and purification need be employed in order to produce two polypeptides which are both antigenically useful.

The fusion polypeptide may comprise one or more AI polypeptide sequences. Preferably, the fusion comprises an AI surface protein sequence. Accordingly, the invention includes a fusion peptide comprising a first amino acid sequence and a second amino acid sequence, wherein said first and second amino acid sequences are selected from a Gram positive bacteria AI protein or a fragment thereof. Preferably, the first and second amino acid sequences in the fusion polypeptide comprise different epitopes.

Hybrids (or fusions) consisting of amino acid sequences from two, three, four, five, six, seven, eight, nine, or ten Gram positive bacteria antigens are preferred. In particular, hybrids consisting of amino acid sequences from two, three, four, or five Gram positive bacteria antigens are preferred.

Different hybrid polypeptides may be mixed together in a single formulation. Within such combinations, a Gram positive bacteria AI sequence may be present in more than one hybrid polypeptide and/or as a non-hybrid polypeptide. It is preferred, however, that an antigen is present either as a hybrid or as a non-hybrid, but not as both.

Hybrid polypeptides can be represented by the formula NH₂-A-{-X-L-}ₙ-B—COOH, wherein: X is an amino acid sequence of a Gram positive bacteria AI sequence or a fragment thereof; L is an optional linker amino acid sequence; A is an optional N-terminal amino acid sequence; B is an optional C-terminal amino acid sequence; and n is 2, 3, 4, 5, 6, 7, 8, 9, 10, 11, 12, 13, 14 or 15.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {—X-L-}, linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. A useful linker is GSGGGG, with the Gly-Ser dipeptide being formed from a BamHI restriction site, thus aiding cloning and manipulation, and the (Gly)₄ tetrapeptide being a typical poly-glycine linker.

-A- is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art.

Most preferably, n is 2 or 3.

### Antibodies: GBS AI Sequences

The GBS AI proteins of the invention may also be used to prepare antibodies specific to the GBS AI proteins. The antibodies are preferably specific to the an oligomeric or hyper-oligomeric form of an AI protein. The invention also includes combinations of antibodies specific to GBS AI proteins selected to provide protection against an increased range of GBS serotypes and strain isolates. For example, a combination may comprise a first and second antibody, wherein said first antibody is specific to a first GBS AI protein and said second antibody is specific to a second GBS AI protein. Preferably, the nucleic acid sequence encoding said first GBS AI protein is not present in a GBS genome comprising a polynucleotide sequence encoding for said second GBS AI protein. Preferably, the nucleic acid sequence encoding said first and second GBS AI proteins are present in the genomes of multiple GBS serotypes and strain isolates.

The GBS specific antibodies of the invention include one or more biological moieties that, through chemical or physical means, can bind to or associate with an epitope of a GBS polypeptide. The antibodies of the invention include antibodies which specifically bind to a GBS AI protein. The invention includes antibodies obtained from both polyclonal and monoclonal preparations, as well as the following: hybrid (chimeric) antibody molecules (see, for example, Winter et al. (1991) Nature 349: 293-299; and U.S. Pat. No. 4,816,567; F(ab′)₂ and F(ab) fragments; Fᵥ molecules (non-covalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5897-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain immunological binding properties of the parent antibody molecule. The invention further includes antibodies obtained through non-conventional processes, such as phage display.

Preferably, the GBS specific antibodies of the invention are monoclonal antibodies. Monoclonal antibodies of the invention include an antibody composition having a homogeneous antibody population. Monoclonal antibodies of the invention may be obtained from murine hybridomas, as well as human monoclonal antibodies obtained using human rather than murine hybridomas. See, e.g., Cote, et al. Monoclonal Antibodies and Cancer Therapy, Alan R. Liss, 1985, p 77.

The antibodies of the invention may be used in diagnostic applications, for example, to detect the presence or absence of GBS in a biological sample. The antibodies of the invention may also be used in the prophylactic or therapeutic treatment of GBS infection.

### Antibodies: Gram Positive Bacteria AI Sequences

The Gram positive bacteria AI proteins of the invention may also be used to prepare antibodies specific to the Gram positive bacteria AI proteins. The antibodies are preferably specific to the an oligomeric or hyper-oligomeric form of an AI protein. The invention also includes combinations of antibodies specific to Gram positive bacteria AI proteins selected to provide protection against an increased range of Gram positive bacteria genus, species, serotypes and strain isolates.

For example, a combination may comprise a first and second antibody, wherein said first antibody is specific to a first Gram positive bacteria AI protein and said second antibody is specific to a second Gram positive bacteria AI protein. Preferably, the nucleic acid sequence encoding said first Gram positive bacteria AI protein is not present in a Gram positive bacterial genome comprising a polynucleotide sequence encoding for said second Gram positive bacteria AI protein. Preferably, the nucleic acid sequence encoding said first and second Gram positive bacteria AI proteins are present in the genomes of multiple Gram positive bacteria genus, species, serotypes or strain isolates.

As an example of an instance where the combination of antibodies provides protection against an increased range of bacteria serotypes, the first antibody may be specific to a first GAS AI protein and the second antibody may be specific to a second GAS AI protein. The first GAS AI protein may comprise a GAS AI-1 surface protein, while the second GAS AI protein may comprise a GAS AI-2 or AI-3 surface protein.

As an example of an instance where the combination of antibodies provides protection against an increased range of bacterial species, the first antibody may be specific to a GBS AI protein and the second antibody may be specific to a GAS AI protein. Alternatively, the first antibody may be specific to a GAS AI protein and the second antibody may be specific to a S. pneumoniae AI protein.

The Gram positive specific antibodies of the invention include one or more biological moieties that, through chemical or physical means, can bind to or associate with an epitope of a Gram positive bacteria AI polypeptide. The antibodies of the invention include antibodies which specifically bind to a Gram positive bacteria AI protein. The invention includes antibodies obtained from both polyclonal and monoclonal preparations, as well as the following: hybrid (chimeric) antibody molecules (see, for example, Winter et al. (1991) Nature 349: 293-299; and U.S. Pat. No. 4,816,567; F(ab′)₂ and F(ab) fragments; Fᵥ molecules (non-covalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5897-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain immunological binding properties of the parent antibody molecule. The invention further includes antibodies obtained through non-conventional processes, such as phage display.

Preferably, the Gram positive specific antibodies of the invention are monoclonal antibodies. Monoclonal antibodies of the invention include an antibody composition having a homogeneous antibody population. Monoclonal antibodies of the invention may be obtained from murine hybridomas, as well as human monoclonal antibodies obtained using human rather than murine hybridomas. See, e.g., Cote, et al. Monoclonal Antibodies and Cancer Therapy, Alan R. Liss, 1985, p 77.

The antibodies of the invention may be used in diagnostic applications, for example, to detect the presence or absence of Gram positive bacteria in a biological sample. The antibodies of the invention may also be used in the prophylactic or therapeutic treatment of Gram positive bacteria infection.

### Nucleic Acids

The invention provides nucleic acids encoding the Gram positive bacteria sequences and/or the hybrid fusion polypeptides of the invention. The invention also provides nucleic acid encoding the GBS antigens and/or the hybrid fusion polypeptides of the invention. Furthermore, the invention provides nucleic acid which can hybridise to these nucleic acids, preferably under “high stringency” conditions (e.g. 65° C. in a 0.1×SSC, 0.5% SDS solution).

Polypeptides of the invention can be prepared by various means (e.g. recombinant expression, purification from cell culture, chemical synthesis, etc.) and in various forms (e.g. native, fusions, non-glycosylated, lipidated, etc.). They are preferably prepared in substantially pure form (i.e. substantially free from other GAS or host cell proteins).

Nucleic acid according to the invention can be prepared in many ways (e.g. by chemical synthesis, from genomic or cDNA libraries, from the organism itself, etc.) and can take various forms (e.g. single stranded, double stranded, vectors, probes, etc.). They are preferably prepared in substantially pure form (i.e. substantially free from other GBS or host cell nucleic acids).

The term “nucleic acid” includes DNA and RNA, and also their analogues, such as those containing modified backbones (e.g. phosphorothioates, etc.), and also peptide nucleic acids (PNA), etc. The invention includes nucleic acid comprising sequences complementary to those described above (e.g. for antisense or probing purposes).

The invention also provides a process for producing a polypeptide of the invention, comprising the step of culturing a host cell transformed with nucleic acid of the invention under conditions which induce polypeptide expression.

The invention provides a process for producing a polypeptide of the invention, comprising the step of synthesising at least part of the polypeptide by chemical means.

The invention provides a process for producing nucleic acid of the invention, comprising the step of amplifying nucleic acid using a primer-based amplification method (e.g. PCR).

The invention provides a process for producing nucleic acid of the invention, comprising the step of synthesising at least part of the nucleic acid by chemical means.

### Purification and Recombinant Expression

The Gram positive bacteria AI proteins of the invention may be isolated from the native Gram positive bacteria, or they may be recombinantly produced, for instance in a heterologous host. For example, the GAS, GBS, and S. pneumoniae antigens of the invention may be isolated from Streptococcus agalactiae, S. pyogenes, S. pneumoniae, or they may be recombinantly produced, for instance, in a heterologous host. Preferably, the GBS antigens are prepared using a heterologous host.

The heterologous host may be prokaryotic (e.g. a bacterium) or eukaryotic. It is preferably E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), S. gordonii, L. lactis, yeasts, etc.

Recombinant production of polypeptides is facilitated by adding a tag protein to the Gram positive bacteria AI sequence to be expressed as a fusion protein comprising the tag protein and the Gram positive bacteria antigen. For example, recombinant production of polypeptides is facilitated by adding a tag protein to the GBS antigen to be expressed as a fusion protein comprising the tag protein and the GBS antigen. Such tag proteins can facilitate purification, detection and stability of the expressed protein. Tag proteins suitable for use in the invention include a polyarginine tag (Arg-tag), polyhistidine tag (His-tag), FLAG-tag, Strep-tag, c-myc-tag, S-tag, calmodulin-binding peptide, cellulose-binding domain, SBP-tag, chitin-binding domain, glutathione S-transferase-tag (GST), maltose-binding protein, transcription termination anti-termination factor (NusA), E. coli thioredoxin (TrxA) and protein disulfide isomerase I (DsbA). Preferred tag proteins include His-tag and GST. A full discussion on the use of tag proteins can be found at Terpe et al., “Overview of tag protein fusions: from molecular and biochemical fundamentals to commercial systems,” Appl Microbiol Biotechnol (2003) 60:523-533.

After purification, the tag proteins may optionally be removed from the expressed fusion protein, i.e., by specifically tailored enzymatic treatments known in the art. Commonly used proteases include enterokinase, tobacco etch virus (TEV), thrombin, and factor Xₐ.

GBS polysaccharides

The compositions of the invention may be further improved by including GBS polysaccharides. Preferably, the GBS antigen and the saccharide each contribute to the immunological response in a recipient. The combination is particularly advantageous where the saccharide and polypeptide provide protection from different GBS serotypes.

The combined antigens may be present as a simple combination where separate saccharide and polypeptide antigens are administered together, or they may be present as a conjugated combination, where the saccharide and polypeptide antigens are covalently linked to each other.

Thus the invention provides an immunogenic composition comprising (i) one or more GBS AI proteins and (ii) one or more GBS saccharide antigens. The polypeptide and the polysaccharide may advantageously be covalently linked to each other to form a conjugate.

Between them, the combined polypeptide and saccharide antigens preferably cover (or provide protection from) two or more GBS serotypes (e.g. 2, 3, 4, 5, 6, 7, 8 or more serotypes). The serotypes of the polypeptide and saccharide antigens may or may not overlap. For example, the polypeptide might protect against serogroup II or V, while the saccharide protects against either serogroups Ia, Ib, or III. Preferred combinations protect against the following groups of serotypes: (1) serotypes Ia and Ib, (2) serotypes Ia and II, (3) serotypes Ia and III, (4) serotypes Ia and IV, (5) serotypes Ia and V, (6) serotypes Ia and VI, (7) serotypes Ia and VII, (8) serotypes Ia and VIII, (9) serotypes Ib and II, (10) serotypes Ib and III, (11) serotypes Ib and IV, (12) serotypes Ib and V, (13) serotypes Ib and VI, (14) serotypes Ib and VII, (15) serotypes Ib and VIII, 16) serotypes II and III, (17) serotypes II and IV, (18) serotypes II and V, (19) serotypes II and VI, (20) serotypes II and VII, (21) serotypes II and VII, (22) serotypes III and IV, (23) serotypes III and V, (24) serotypes III and VI, (25) serotypes III and VII, (26) serotypes III and VIII, (27) serotypes IV and V, (28) serotypes IV and VI, (29) serotypes IV and VII, (30) serotypes IV and VIII, (31) serotypes V and VI, (32) serotypes V and VII, (33) serotypes V and VIII, (34) serotypes VI and VII, (35) serotypes VI and VIII, and (36) serotypes VII and VIII.

Still more preferably, the combinations protect against the following groups of serotypes: (1) serotypes Ia and II, (2) serotypes Ia and V, (3) serotypes Ib and II, (4) serotypes Ib and V, (5) serotypes III and II, and (6) serotypes III and V. Most preferably, the combinations protect against serotypes III and V.

Protection against serotypes II and V is preferably provided by polypeptide antigens. Protection against serotypes Ia, Ib and/or III may be polypeptide or saccharide antigens.

### Immunogenic Compositions and Medicaments

Compositions of the invention are preferably immunogenic compositions, and are more preferably vaccine compositions. The pH of the composition is preferably between 6 and 8, preferably about 7. The pH may be maintained by the use of a buffer. The composition may be sterile and/or pyrogen-free. The composition may be isotonic with respect to humans.

Vaccines according to the invention may either be prophylactic (i.e. to prevent infection) or therapeutic (i.e. to treat infection), but will typically be prophylactic. Accordingly, the invention includes a method for the therapeutic or prophylactic treatment of a Gram positive bacteria infection in an animal susceptible to such gram positive bacterial infection comprising administering to said animal a therapeutic or prophylactic amount of the immunogenic composition of the invention. For example, the invention includes a method for the therapeutic or prophylactic treatment of a Streptococcus agalactiae, S. pyogenes, or S. pneumoniae infection in an animal susceptible to streptococcal infection comprising administering to said animal a therapeutic or prophylactic amount of the immunogenic compositions of the invention.

The invention also provides a composition of the invention for use of the compositions described herein as a medicament. The medicament is preferably able to raise an immune response in a mammal (i.e. it is an immunogenic composition) and is more preferably a vaccine.

The invention also provides the use of the compositions of the invention in the manufacture of a medicament for raising an immune response in a mammal The medicament is preferably a vaccine.

The invention also provides kits comprising one or more containers of compositions of the invention. Compositions can be in liquid form or can be lyophilized, as can individual antigens. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The composition may comprise a first component comprising one or more Gram positive bacteria AI proteins. Preferably, the AI proteins are surface AI proteins. Preferably, the AI surface proteins are in an oligomeric or hyperoligomeric form. For example, the first component comprises a combination of GBS antigens or GAS antigens, or S. pneumoniae antigens. Preferably said combination includes GBS 80. Preferably GBS 80 is present in an oligomeric or hyperoligomeric form.

The kit can further comprise a second container comprising a pharmaceutically-acceptable buffer, such as phosphate-buffered saline, Ringer's solution, or dextrose solution. It can also contain other materials useful to the end-user, including other buffers, diluents, filters, needles, and syringes. The kit can also comprise a second or third container with another active agent, for example an antibiotic.

The kit can also comprise a package insert containing written instructions for methods of inducing immunity against S agalactiae and or S. pyogenes and/or S pneumoniae or for treating S agalactiae and or S. pyogenes and/or S pneumoniae infections. The package insert can be an unapproved draft package insert or can be a package insert approved by the Food and Drug Administration (FDA) or other regulatory body.

The invention also provides a delivery device pre-filled with the immunogenic compositions of the invention.

The invention also provides a method for raising an immune response in a mammal comprising the step of administering an effective amount of a composition of the invention. The immune response is preferably protective and preferably involves antibodies and/or cell-mediated immunity This immune response will preferably induce long lasting (e.g., neutralising) antibodies and a cell mediated immunity that can quickly respond upon exposure to one or more GBS and/or GAS and/or S. pneumoniae antigens. The method may raise a booster response.

The invention provides a method of neutralizing GBS, GAS, or S. pneumoniae infection in a mammal comprising the step of administering to the mammal an effective amount of the immunogenic compositions of the invention, a vaccine of the invention, or antibodies which recognize an immunogenic composition of the invention.

The mammal is preferably a human. Where the vaccine is for prophylactic use, the human is preferably a female (either of child bearing age or a teenager). Alternatively, the human may be elderly (e.g., over the age of 50, 55, 60, 65, 70 or 75) and may have an underlying disease such as diabetes or cancer. Where the vaccine is for therapeutic use, the human is preferably a pregnant female or an elderly adult.

These uses and methods are preferably for the prevention and/or treatment of a disease caused by Streptococcus agalactiae, or S. pyogenes, or S. pneumoniae. The compositions may also be effective against other streptococcal bacteria. The compositions may also be effective against other Gram positive bacteria.

One way of checking efficacy of therapeutic treatment involves monitoring Gram positive bacterial infection after administration of the composition of the invention. One way of checking efficacy of prophylactic treatment involves monitoring immune responses against the Gram positive bacterial antigens in the compositions of the invention after administration of the composition.

One way of checking efficacy of therapeutic treatment involves monitoring GBS infection after administration of the composition of the invention. One way of checking efficacy of prophylactic treatment involves monitoring immune responses against the GBS antigens in the compositions of the invention after administration of the composition.

A way of assessing the immunogenicity of the component proteins of the immunogenic compositions of the present invention is to express the proteins recombinantly and to screen patient sera or mucosal secretions by immunoblot. A positive reaction between the protein and the patient serum indicates that the patient has previously mounted an immune response to the protein in question—that is, the protein is an immunogen. This method may also be used to identify immunodominant proteins and/or epitopes.

Another way of checking efficacy of therapeutic treatment involves monitoring GBS or GAS or S pneumoniae infection after administration of the compositions of the invention. One way of checking efficacy of prophylactic treatment involves monitoring immune responses both systemically (such as monitoring the level of IgG1 and IgG2a production) and mucosally (such as monitoring the level of IgA production) against the GBS and/or GAS and/or S pneumoniae antigens in the compositions of the invention after administration of the composition. Typically, GBS and/or GAS and/or S pneumoniae serum specific antibody responses are determined post-immunization but pre-challenge whereas mucosal GBS and/or GAS and/or S pneumoniae specific antibody body responses are determined post-immunization and post-challenge.

The vaccine compositions of the present invention can be evaluated in in vitro and in vivo animal models prior to host, e.g., human, administration.

The efficacy of immunogenic compositions of the invention can also be determined in vivo by challenging animal models of GBS and/or GAS and/or S pneumoniae infection, e.g., guinea pigs or mice, with the immunogenic compositions. The immunogenic compositions may or may not be derived from the same serotypes as the challenge serotypes. Preferably the immunogenic compositions are derivable from the same serotypes as the challenge serotypes. More preferably, the immunogenic composition and/or the challenge serotypes are derivable from the group of GBS and/or GAS and/or S pneumoniae serotypes.

In vivo efficacy models include but are not limited to: (i) A murine infection model using human GBS and/or GAS and/or S pneumoniae serotypes; (ii) a murine disease model which is a murine model using a mouse-adapted GBS and/or GAS and/or S pneumoniae strain, such as those strains outlined above which is particularly virulent in mice and (iii) a primate model using human GBS or GAS or S pneumoniae isolates.

The immune response may be one or both of a TH1 immune response and a TH2 response.

The immune response may be an improved or an enhanced or an altered immune response.

The immune response may be one or both of a systemic and a mucosal immune response.

Preferably the immune response is an enhanced system and/or mucosal response.

An enhanced systemic and/or mucosal immunity is reflected in an enhanced TH1 and/or TH2 immune response. Preferably, the enhanced immune response includes an increase in the production of IgG1 and/or IgG2a and/or IgA

Preferably the mucosal immune response is a TH2 immune response. Preferably, the mucosal immune response includes an increase in the production of IgA.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH1 immune response may include one or more of an increase in CTLs, an increase in one or more of the cytokines associated with a TH1 immune response (such as IL-2, IFN-γ, and TNFβ), an increase in activated macrophages, an increase in NK activity, or an increase in the production of IgG2a. Preferably, the enhanced TH1 immune response will include an increase in IgG2a production.

Immunogenic compositions of the invention, in particular, immunogenic composition comprising one or more GAS antigens of the present invention may be used either alone or in combination with other GAS antigens optionally with an immunoregulatory agent capable of eliciting a Th1 and/or Th2 response.

Compositions of the invention will generally be administered directly to a patient. Certain routes may be favored for certain compositions, as resulting in the generation of a more effective immune response, preferably a CMI response, or as being less likely to induce side effects, or as being easier for administration. Direct delivery may be accomplished by parenteral injection (e.g. subcutaneously, intraperitoneally, intradermally, intravenously, intramuscularly, or to the interstitial space of a tissue), or by rectal, oral (e.g. tablet, spray), vaginal, topical, transdermal (e.g. see WO 99/27961) or transcutaneous (e.g. see WO 02/074244 and WO 02/064162), intranasal (e.g. see WO03/028760), ocular, aural, pulmonary or other mucosal administration.

The invention may be used to elicit systemic and/or mucosal immunity

In one particularly preferred embodiment, the immunogenic composition comprises one or more GBS or GAS or S pneumoniae antigen(s) which elicits a neutralising antibody response and one or more GBS or GAS or S pneumoniae antigen(s) which elicit a cell mediated immune response. In this way, the neutralising antibody response prevents or inhibits an initial GBS or GAS or S pneumoniae infection while the cell-mediated immune response capable of eliciting an enhanced Th1 cellular response prevents further spreading of the GBS or GAS or S pneumoniae infection. Preferably, the immunogenic composition comprises one or more GBS or GAS or S pneumoniae surface antigens and one or more GBS or GAS or S pneumoniae cytoplasmic antigens. Preferably the immunogenic composition comprises one or more GBS or GAS or S pneumoniae surface antigens or the like and one or other antigens, such as a cytoplasmic antigen capable of eliciting a Th1 cellular response.

Dosage treatment can be a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc.

The compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Immunogenic compositions used as vaccines comprise an immunologically effective amount of antigen(s), as well as any other components, such as antibiotics, as needed. By ‘immunologically effective amount,’ it is meant that the administration of that amount to an individual, either in a single dose or as part of a series, is effective for treatment or prevention, or increases a measurable immune response or prevents or reduces a clinical symptom. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

### Further Components of the Composition

The composition of the invention will typically, in addition to the components mentioned above, comprise one or more ‘pharmaceutically acceptable carriers,’ which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in Gennaro (2000) Remington: The Science and Practice of Pharmacy. 20th ed., ISBN: 0683306472.

### Adjuvants

Vaccines of the invention may be administered in conjunction with other immunoregulatory agents. In particular, compositions will usually include an adjuvant. Adjuvants for use with the invention include, but are not limited to, one or more of the following set forth below:

A. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (e.g. see chapters 8 & 9 of Vaccine Design . . . (1995) eds. Powell & Newman. ISBN: 030644867X. Plenum.), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO 00/23105).

Aluminum salts may be included in vaccines of the invention such that the dose of Al³⁺ is between 0.2 and 1.0 mg per dose.

B. Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See WO90/14837. See also, Podda, “The adjuvanted influenza vaccines with novel adjuvants: experience with the MF59-adjuvanted vaccine,” Vaccine (2001) 19: 2673-2680; Frey et al., “Comparison of the safety, tolerability, and immunogenicity of a MF59-adjuvanted influenza vaccine and a non-adjuvanted influenza vaccine in non-elderly adults,” Vaccine (2003) 21:4234-4237. MF59 is used as the adjuvant in the FLUAD™ influenza virus trivalent subunit vaccine.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyelthylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-sn-glycero-3-huydroxyphosphophoryloxy)-ethylamine (MTP-PE), for example, the submicron oil-in-water emulsion known as “MF59” (International Publication No. WO 90/14837; U.S. Pat. Nos. 6,299,884 and 6,451,325, incorporated herein by reference in their entireties; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO 90/14837 and U.S. Pat. Nos. 6,299,884 and 6,451,325, incorporated herein by reference in their entireties.

Complete Freund's adjuvant (CFA) and incomplete Freund's adjuvant (IFA) may also be used as adjuvants in the invention.

C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-LC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO 96/11711 and WO 96/33739. Optionally, the ISCOMS may be devoid of additional detergent. See WO 00/07621.

A review of the development of saponin based adjuvants can be found at Barr, et al., “ISCOMs and other saponin based adjuvants,” Advanced Drug Delivery Reviews (1998) 32:247-271. See also Sjolander, et al., “Uptake and adjuvant activity of orally delivered saponin and ISCOM vaccines,” Advanced Drug Delivery Reviews (1998) 32:321-338.

D. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO 03/024480, WO 03/024481, and Niikura et al., “Chimeric Recombinant Hepatitis E Virus-Like Particles as an Oral Vaccine Vehicle Presenting Foreign Epitopes,” Virology (2002) 293:273-280; Lenz et al., “Papillomarvirus-Like Particles Induce Acute Activation of Dendritic Cells,” Journal of Immunology (2001) 5246-5355; Pinto, et al., “Cellular Immune Responses to Human Papillomavirus (HPV)-16 L1 Healthy Volunteers Immunized with Recombinant HPV-16 L1 Virus-Like Particles,” Journal of Infectious Diseases (2003) 188:327-338; and Gerber et al., “Human Papillomavirus Virus-Like Particles Are Efficient Oral Immunogens when Coadministered with Escherichia coli Heat-Labile Entertoxin Mutant R192G or CpG,” Journal of Virology (2001) 75(10):4752-4760. Virosomes are discussed further in, for example, Gluck et al., “New Technology Platforms in the Development of Vaccines for the Future,” Vaccine (2002) 20:B10-B16 Immunopotentiating reconstituted influenza virosomes (IRIV) are used as the subunit antigen delivery system in the intranasal trivalent INFLEXAL™ product {Mischler & Metcalfe (2002) Vaccine 20 Suppl 5:B17-23} and the INFLUVAC PLUS™ product.

E. Bacterial or Microbial Derivatives

Adjuvants suitable for use in the invention include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.

(2) Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei,” Vaccine (2003) 21:2485-2491; and Pajak, et al., “The Adjuvant OM-174 induces both the migration and maturation of murine dendritic cells in vivo,” Vaccine (2003) 21:836-842.

(3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles,” Nucleic Acids Research (2003) 31(9): 2393-2400; WO02/26757 and WO99/62923 for examples of possible analog substitutions. The adjuvant effect of CpG oligonucleotides is further discussed in Krieg, “CpG motifs: the active ingredient in bacterial extracts?,” Nature Medicine (2003) 9(7): 831-835; McCluskie, et al., “Parenteral and mucosal prime-boost immunization strategies in mice with hepatitis B surface antigen and CpG DNA,” FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See Kandimalla, et al., “Toll-like receptor 9: modulation of recognition and cytokine induction by novel synthetic CpG DNAs,” Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha,” J. Immunol (2003) 170(8):4061-4068; Krieg, “From A to Z on CpG,” TRENDS in Immunology (2002) 23(2): 64-65 and WO01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., “Secondary structures in CpG oligonucleotides affect immunostimulatory activity,” BBRC (2003) 306:948-953; Kandimalla, et al., “Toll-like receptor 9: modulation of recognition and cytokine induction by novel synthetic GpG DNAs,” Biochemical Society Transactions (2003) 31(part 3):664-658; Bhagat et al., “CpG penta- and hexadeoxyribonucleotides as potent immunomodulatory agents” BBRC (2003) 300:853-861 and WO 03/035836.

(4) ADP-Ribosylating Toxins and Detoxified Derivatives Thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references, each of which is specifically incorporated by reference herein in their entirety: Beignon, et al., “The LTR72 Mutant of Heat-Labile Enterotoxin of Escherichia coli Enhances the Ability of Peptide Antigens to Elicit CD4+ T Cells and Secrete Gamma Interferon after Coapplication onto Bare Skin,” Infection and Immunity (2002) 70(6):3012-3019; Pizza, et al., “Mucosal vaccines: non toxic derivatives of LT and CT as mucosal adjuvants,” Vaccine (2001) 19:2534-2541; Pizza, et al., “LTK63 and LTR72, two mucosal adjuvants ready for clinical trials” Int. J. Med. Microbiol (2000) 290(4-5):455-461; Scharton-Kersten et al., “Transcutaneous Immunization with Bacterial ADP-Ribosylating Exotoxins, Subunits and Unrelated Adjuvants,” Infection and Immunity (2000) 68(9):5306-5313; Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides,” Immunol Lett. (1999) 67(3):209-216; Peppoloni et al., “Mutants of the Escherichia coli heat-labile enterotoxin as safe and strong adjuvants for intranasal delivery of vaccines,” Vaccines (2003) 2(2):285-293; and Pine et al., (2002) “Intranasal immunization with influenza vaccine and a detoxified mutant of heat labile enterotoxin from Escherichia coli (LTK63)” J. Control Release (2002) 85(1-3):263-270. Numerical reference for amino acid substitutions is preferably based on the alignments of the A and B subunits of ADP-ribosylating toxins set forth in Domenighini et al., Mol. Microbiol (1995) 15(6):1165-1167, specifically incorporated herein by reference in its entirety.

F. Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Rele. 70:267-276) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention. E.g. WO99/27960.

G. Microparticles

Microparticles may also be used as adjuvants in the invention. Microparticles (i.e. a particle of ˜100 nm to ˜150 μm in diameter, more preferably ˜200 nm to —30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

H. Liposomes

Examples of liposome formulations suitable for use as adjuvants are described in U.S. Pat. No. 6,090,406, U.S. Pat. No. 5,916,588, and EP 0 626 169.

I. Polyoxyethylene ether and Polyoxyethylene Ester Formulations

Adjuvants suitable for use in the invention include polyoxyethylene ethers and polyoxyethylene esters. WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO 01/21152).

Preferred polyoxyethylene ethers are selected from the following group: polyoxyethylene-9-lauryl ether(laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

J. Polyphosphazene (PCPP)

PCPP formulations are described, for example, in Andrianov et al., “Preparation of hydrogel microspheres by coacervation of aqueous polyphophazene solutions,” Biomaterials (1998) 19(1-3):109-115 and Payne et al., “Protein Release from Polyphosphazene Matrices,” Adv. Drug. Delivery Review (1998) 31(3):185-196.

K. Muramyl Peptides

Examples of muramyl peptides suitable for use as adjuvants in the invention include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

L. Imidazoquinolone Compounds.

Examples of imidazoquinolone compounds suitable for use adjuvants in the invention include Imiquamod and its homologues, described further in Stanley, “Imiquimod and the imidazoquinolones: mechanism of action and therapeutic potential” Clin Exp Dermatol (2002) 27(7):571-577 and Jones, “Resiquimod 3M,” Curr Opin Investig Drugs (2003) 4(2):214-218.

The invention may also comprise combinations of aspects of one or more of the adjuvants identified above. For example, the following adjuvant compositions may be used in the invention:
(1) a saponin and an oil-in-water emulsion (WO 99/11241);
(2) a saponin (e.g., QS21)+a non-toxic LPS derivative (e.g. 3dMPL) (see WO 94/00153);
(3) a saponin (e.g., QS21)+a non-toxic LPS derivative (e.g. 3dMPL)+a cholesterol;
(4) a saponin (e.g. QS21)+3dMPL+IL-12 (optionally+a sterol) (WO 98/57659);
(5) combinations of 3dMPL with, for example, QS21 and/or oil-in-water emulsions (See European patent applications 0835318, 0735898 and 0761231);
(6) SAF, containing 10% Squalane, 0.4% Tween 80, 5% pluronic-block polymer L121, and thr-MDP, either microfluidized into a submicron emulsion or vortexed to generate a larger particle size emulsion.
(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™);
(8) one or more mineral salts (such as an aluminum salt)+a non-toxic derivative of LPS (such as 3dPML).
(9) one or more mineral salts (such as an aluminum salt)+an immunostimulatory oligonucleotide (such as a nucleotide sequence including a CpG motif). Combination No. (9) is a preferred adjuvant combination.

M. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

Aluminum salts and MF59 are preferred adjuvants for use with injectable influenza vaccines. Bacterial toxins and bioadhesives are preferred adjuvants for use with mucosally-delivered vaccines, such as nasal vaccines.

The immunogenic compositions of the present invention may be administered in combination with an antibiotic treatment regime. In one embodiment, the antibiotic is administered prior to administration of the antigen of the invention or the composition comprising the one or more of the antigens of the invention.

In another embodiment, the antibiotic is administered subsequent to the administration of the one or more antigens of the invention or the composition comprising the one or more antigens of the invention. Examples of antibiotics suitable for use in the treatment of the Steptococcal infections of the invention include but are not limited to penicillin or a derivative thereof or clindamycin or the like.

### Further Antigens

The compositions of the invention may further comprise one or more additional Gram positive bacterial antigens which are not associated with an AI. Preferably, the Gram positive bacterial antigens that are not associated with an AI can provide protection across more than one serotype or strain isolate. For example, a first non-AI antigen, in which the first non-AI antigen is at least 90% (i.e., at least 90, 91, 92, 93, 94, 95, 96, 97, 98, 99, or 100%) homologous to the amino acid sequence of a second non-AI antigen, wherein the first and the second non-AI antigen are derived from the genomes of different serotypes of a Gram positive bacteria, may be further included in the compositions. The first non-AI antigen may also be homologous to the amino acid sequence of a third non-AI antigen, such that the first non-AI antigen, the second non-AI antigen, and the third non-AI antigen are derived from the genomes of different serotypes of a Gram positive bacteria. The first non-AI antigen may also be homologous to the amino acid sequence of a fourth non-AI antigen, such that the first non-AI antigen, the second non-AI antigen, the third non-AI antigen, and the fourth non-AI antigen are derived from the genomes of different serotypes of a Gram positive bacteria.

The first non-AI antigen may be GBS 322. The amino acid sequence of GBS 322 across GBS strains from serotypes Ia, Ib, II, III, V, and VIII is greater than 90%. Alternatively, the first non-AI antigen may be GBS 276. The amino acid sequence of GBS 276 across GBS strain from serotypes Ia, Ib, II, III, V, and VIII is greater than 90%. Table 13 provides the percent amino acid sequence identity of GBS 322 and GBS 276 across different GBS strains and serotypes.

TABLE 13
Conservation of GBS 322 and GBS 276 amino acid sequences

| | | GBS 322 | | GBS 276 | |
|---|---|---|---|---|---|
| Serotype | Strains | cGH | % AA identity | cGH | % AA identity |
| Ia | 090 | + | 98.60 | + | 97.90 |
| | A909 | + | 98.30 | + | 97.90 |
| | 515 | + | 98.80 | + | 97.50 |
| | DK1 | + | | + | |
| | DK8 | + | | + | |
| | Davis | + | | + | |
| Ib | 7357b | + | | + | |
| | H36B | + | 98.30 | + | 97.80 |
| II | 18RS21 | + | 100.00 | + | 99.90 |
| | DK21 | + | | + | |
| III | NEM316 | + | 100.00 | + | 97.00 |
| | COH31 | + | | + | |
| | D136 | + | | + | |
| | M732 | + | 98.00 | + | 100.00 |
| | COH1 | + | 98.30 | + | 100.00 |
| | M781 | + | 98.30 | + | 99.60 |
| No type | CJB110 | + | 98.60 | + | 97.90 |
| | 1169NT | + | 97.40 | + | 97.90 |
| V | CJB111 | + | 100.00 | + | |
| | 2603 | + | 100.00 | + | 100.00 |
| VIII | JM130013 | + | 100.00 | + | 97.90 |
| | SMU014 | + | | + | |
| total | | 22/22 | 98.28 +/− 0.4 | 22/22 | 98.44 +/− 1.094 |

As an example, inclusion of a non-AI protein, GBS 322, in combination with AI antigens GBS 67, GBS 80, and GBS 104 provided protection to newborn mice in an active maternal immunization assay.

TABLE 14
Active maternal immunization assay for a combination of fragments from
GBS 322, GBS 80, GBS 104, and GBS 67

| | | FACS (Δ Mean) | | | MIX = 322 + 80 + 104 + 67 | | PBS | |
|---|---|---|---|---|---|---|---|---|
| GBS strains | Type | GBS 80 | GBS 67 | GBS 322 | alive/treated | % protection | alive/treated | % protection |
| 515 | Ia | 0 | 409 | 227 | 39/40 | 97 | 6/40 | 15 |
| 7357b- | Ib | 91 | 316 | 102 | 19/30 | 63 | 1/30 | 3 |
| DK21 | II | 0 | 331 | 416 | 25/34 | 73 | 17/48 | 35 |
| 5401 | II | 170 | 618 | 135 | 35/40 | 87 | 3/37 | 8 |
| 3050 | II | 43 | 460 | 188 | 48/48 | 100 | 1/30 | 3 |
| COH1 | III | 305 | 0 | 130 | 36/36 | 100 | 7/40 | 17 |
| M781 | III | 65 | 0 | 224 | 30/40 | 75 | 4/39 | 10 |
| 2603 | V | 125 | 105 | 313 | 27/33 | 82 | 10/35 | 28 |
| CJB111 | V | 370 | 481 | 63 | 25/28 | 89 | 4/46 | 9 |
| JM9130013 | VIII | 597 | 83 | 143 | 37/39 | 95 | 5/40 | 12 |
| JMU071 | VIII | 556 | 79 | 170 | 44/50 | 88 | 18/50 | 36 |
| NT1169 | NT | 0 | 443 | 213 | 12/32 | 37 | 11/35 | 31 |

In fact, the non-AI GBS 322 antigen may itself provide protection to newborn mice in an active maternal immunization assay.

TABLE 16
Active maternal immunization assay for each of
GBS 80 and GBS 322 antigens

| | | GBS 80 | | | GBS 322 | | |
|---|---|---|---|---|---|---|---|
| | | | Protection | | | Protection | |
| | | FACS | (% survival) | | FACS | (% survival) | |
| GBS strains | Type | Δ Mean | antigen | ctrl- | Δ Mean | antigen | ctrl- |
| CJB111 | V | 370 | 72% | 40% | 63 | 57% | 40% |
| COH1 | III | 305 | 76% | 10% | 130 | 3% | 10% |
| 2603 | V | 82 | 22% | 34% | 313 | 83% | 34% |
| 7357b- | Ib | 91 | 36% | 34% | 102 | 43% | 34% |
| 18RS21 | II | 0 | 15% | 24% | 268 | 84% | 24% |
| DK21 | II | 0 | 10% | 21% | 416 | 67% | 25% |
| A909 | Ia | 0 | 0% | 14% | | | |
| 090 | Ia | 0 | 0% | 0% | | | |
| H36B | Ib | | | | 105 | 34% | 32% |

Thus, inclusion of a non-AI protein in an immunogenic composition of the invention may provide increased protection a mammal.

The immunogenic compositions comprising S. pneumoniae AI polypeptides may further secondary SP protein antigens which include (a) any of the SP protein antigens disclosed in WO 02/077021 or U.S. provisional application ______, filed Apr. 20, 2005 (Attorney Docket Number 002441.00154), (2) immunogenic portions of the antigens comprising at least 7 contiguous amino acids, (3) proteins comprising amino acid sequences which retain immunogenicity and which are at least 95% identical to these SP protein antigens (e.g., 95%, 96%, 97%, 98%, 99%, or 99.5% identical), and (4) fusion proteins, including hybrid SP protein antigens, comprising (1)-(3).

Alternatively, the invention may include an immunogenic composition comprising a first and a second Gram positive bacteria non-AI protein, wherein the polynucleotide sequence encoding the sequence of the first non-AI protein is less than 90% (i.e., less than 90, 88, 86, 84, 82, 81, 78, 76, 74, 72, 70, 65, 60, 55, 50, 45, 40, 35, or 30 percent) homologous than the corresponding sequence in the genome of the second non-AI protein.

The compositions of the invention may further comprise one or more additional non-Gram positive bacterial antigens, including additional bacterial, viral or parasitic antigens. The compositions of the invention may further comprise one or more additional non-GBS antigens, including additional bacterial, viral or parasitic antigens.

In another embodiment, the GBS antigen combinations of the invention are combined with one or more additional, non-GBS antigens suitable for use in a vaccine designed to protect elderly or immunocomprised individuals. For example, the GBS antigen combinations may be combined with an antigen derived from the group consisting of Enterococcus faecalis, Staphylococcus aureus, Staphylococcus epidermis, Pseudomonas aeruginosa, Legionella pneumophila, Listeria monocytogenes, Neisseria meningitides, influenza, and Parainfluenza virus (‘PIV’).

Where a saccharide or carbohydrate antigen is used, it is preferably conjugated to a carrier protein in order to enhance immunogenicity {e.g. Ramsay et al. (2001) Lancet 357(9251):195-196; Lindberg (1999) Vaccine 17 Suppl 2:S28-36; Buttery & Moxon (2000) J R Coll Physicians Lond 34:163-168; Ahmad & Chapnick (1999) Infect Dis Clin North Am 13:113-133, vii.; Goldblatt (1998) J. Med. Microbiol. 47:563-567; European patent 0 477 508; U.S. Pat. No. 5,306,492; International patent application WO98/42721; Conjugate Vaccines (eds. Cruse et al.) ISBN 3805549326, particularly vol. 10:48-114; and Hermanson (1996) Bioconjugate Techniques ISBN: 0123423368 or 012342335X}. Preferred carrier proteins are bacterial toxins or toxoids, such as diphtheria or tetanus toxoids. The CRM₁₉₇ diphtheria toxoid is particularly preferred {Research Disclosure, 453077 (January 2002)}. Other carrier polypeptides include the N. meningitidis outer membrane protein (EP-A-0372501), synthetic peptides (EP-A-0378881; EP-A-0427347), heat shock proteins (WO 93/17712; WO 94/03208), pertussis proteins (WO 98/58668; EP A 0471177), protein D from H. influenzae (WO 00/56360), cytokines (WO 91/01146), lymphokines, hormones, growth factors, toxin A or B from C. difficile (WO00/61761), iron-uptake proteins (WO01/72337), etc. Where a mixture comprises capsular saccharides from both serogroups A and C, it may be preferred that the ratio (w/w) of MenA saccharide:MenC saccharide is greater than 1 (e.g. 2:1, 3:1, 4:1, 5:1, 10:1 or higher). Different saccharides can be conjugated to the same or different type of carrier protein. Any suitable conjugation reaction can be used, with any suitable linker where necessary.

Toxic protein antigens may be detoxified where necessary e.g. detoxification of pertussis toxin by chemical and/or genetic means.

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens in the composition will typically be present at a concentration of at least 1 μg/ml each. In general, the concentration of any given antigen will be sufficient to elicit an immune response against that antigen.

As an alternative to using protein antigens in the composition of the invention, nucleic acid encoding the antigen may be used {e.g. refs. Robinson & Torres (1997) Seminars in Immunology 9:271-283; Donnelly et al. (1997) Annu Rev Immunol 15:617-648; Scott-Taylor & Dalgleish (2000) Expert Opin Investig Drugs 9:471-480; Apostolopoulos & Plebanski (2000) Curr Opin Mol Ther 2:441-447; Ilan (1999) Curr Opin Mol Ther 1:116-120; Dubensky et al. (2000) Mol Med 6:723-732; Robinson & Pertmer (2000) Adv Virus Res 55:1-74; Donnelly et al. (2000) Am J Respir Crit Care Med 162(4 Pt 2):S190-193; and Davis (1999) Mt. Sinai J. Med. 66:84-90}. Protein components of the compositions of the invention may thus be replaced by nucleic acid (preferably DNA e.g. in the form of a plasmid) that encodes the protein.

### Definitions

The term “comprising” means “including” as well as “consisting” e.g. a composition “comprising” X may consist exclusively of X or may include something additional e.g. X+Y.

The term “about” in relation to a numerical value x means, for example, x+10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of Current Protocols in Molecular Biology (F. M. Ausubel et al., eds., 1987) Supplement 30. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489.

The invention is further illustrated, without limitation, by the following examples.

### Example 1

### Binding of an Adhesin Island Surface Protein, GBS 80, to Fibrinogen and Fibronectin

This example demonstrates that an Adhesin Island surface protein, GBS 80 can bind to fibrinogen and fibronectin.

An enzyme-linked immunosorbent assay (ELISA) was used to analyse the in vitro binding ability of recombinant GBS 80 to immobilized extra-cellular matrix (ECM) proteins but not to bovine serum albumin (BSA). Microtiter plates were coated with ECM proteins (fibrinogen, fibronectin, laminin, collagen type IV) and binding assessed by adding varying concentrations of a recombinant form of GBS 80, over-expressed and purified from E. coli (FIG. 5A). Plates were then incubated sequentially with a) mouse anti-GBS 80 primary antibody; b) rabbit anti-mouse AP-conjugated secondary antibody; c) pNPP colorimetric substrate. Relative binding was measured by monitoring absorbance at 405 nm, using 595 nm as a reference wavelength. FIG. 5b shows binding of recombinant GBS 80 to immobilized ECM proteins (1 μg) as a function of concentration of GBS 80. BSA was used as a negative control. Data points represent the means of OD₄₀₅ values±standard deviation for 3 wells.

Binding of GBS 80 to the tested ECM proteins was found to be concentration dependent and exhibited saturation kinetics. As is also evident from FIG. 5, binding of GBS 80 to fibronectin and fibrinogen was greater than binding to laminin and collagen type IV at all the concentrations tested.

### Example 2

### GBS 80 is Required for Surface Localization of GBS 104

This example demonstrates that co-expression of GBS 80 is required for surface localization of GBS 104.

The polycistronic nature of the Adhesin Island I mRNA was investigated through reverse transcriptase-PCR (RT-PCR) analysis employing primers designed to detect transcripts arising from contiguous genes. Total RNA was isolated from GBS cultures grown to an optical density at 600 nm (OD₆₀₀) of 0.3 in THB (Todd-Hewitt broth) by the RNeasy Total RNA isolation method (Qiagen) according to the manufacturer's instructions. The absence of contaminating chromosomal DNA was confirmed by failure of the gene amplification reactions to generate a product detectable by agarose gel electrophoresis, in the absence of reverse transcriptase. RT-PCR analysis was performed with the Access RT-PCR system (Promega) according to the manufacturer's instructions, employing PCR cycling temperatures of 60° C. for annealing and 70° C. for extension. Amplification products were visualized alongside 100-bp DNA markers in 2% agarose gels after ethidium bromide staining.

FIG. 5 shows that all the genes are co-transcribed as an operon. A schematic of the AI-1 operon is shown above the agarose gel analysis of the RT-PCR products. Large rectangular arrows indicate the predicted transcript direction. Primer pairs were selected such as “1-4” cross the 3′ finish-5′ start of successive genes and overlap each gene by at least 200 bp. Additionally, “1” crosses a putative rho-independent transcriptional terminator. “5” is an internal GBS 80 control and “6” is an unrelated control from a highly expressed gene. Lanes: “a”: RNA plus RTase enzyme; “b” RNA without RTase; “c”: genomic DNA control.

In the effort to elucidate the functions of the AI-1 proteins, in frame deletions of all of the genes within the operon have been constructed and the resulting mutants characterized with respect to surface exposure of the encoded antigens (see FIG. 8).

Each in-frame deletion mutation was constructed by splice overlap extension PCR (SOE-PCR) essentially as described by Horton et al. [Horton R. M., Z. L. Cai, S. N. Ho, L. R. Pease (1990) Biotechniques 8:528-35] using suitable primers and cloned into the temperature sensitive shuttle vector pJRS233 to replace the wild type copy by allelic exchange [Perez-Casal, J., J. A. Price, et al. (1993) Mol Microbiol 8(5): 809-19.]. All plasmid constructions utilized standard molecular biology techniques, and the identities of DNA fragments generated by PCR were verified by sequencing. Following SOE-PCR, the resulting mutant DNA fragments were digested with XhoI and EcoRI, and ligated into a similarly digested pJRS233. The resulting vectors were introduced by electroporation into the chromosome of 2603 and COH1 GBS strains in a three-step process, essentially as described in Framson et al. [Framson, P. E., A. Nittayajarn, J. Merry, P. Youngman, and C. E. Rubens. (1997) Appl. Environ. Microbiol. 63(9):3539-47]. Briefly, the vector pJRS233 contains an erm gene encoding erythromycin resistance and a temperature-sensitive gram-positive replicon that is active at 30° C. but not at 37° C. Initially, the constructs are electroporated into GBS electro-competent cells prepared as described by Frameson et al., and transformants containing free plasmid are selected by their ability to grow at 30° C. on Todd-Hewitt Broth (THB) agar plates containing 1 μg/ml erythromycin. The second step includes a selection step for strains in which the plasmid has integrated into the chromosome via a single recombination event over the homologous plasmid insert and chromosome sequence by their ability to grow at 37° C. on THB agar medium containing 1 mg/ml erythromycin. In the third step, GBS cells containing the plasmid integrated within the chromosome (integrants) are serially passed in broth culture in the absence of antibiotics at 30° C. Plasmid excision from the chromosome via a second recombination event over the duplicated target gene sequence either completed the allelic exchange or reconstituted the wild-type genotype. Subsequent loss of the plasmid in the absence of antibiotic selection pressure resulted in an erythromycin-sensitive phenotype. In order to assess gene replacement a screening of erythromycin-sensitive colonies was performed by analysis of the target gene PCR amplicons.

FIG. 7 reports a schematic of the IS-1 operon for each knock-out strain generated, along with the deletion position within the amino acidic sequence. Most data presented here concern the COH1 deletion strains, in which the expression of each of the antigens is higher by DNA microarray analysis as well as detectable by FACS analysis (see FIG. 8). The double mutant in 2603 Δ80, Δ04 double mutant was constructed by sequential allelic exchanges of the shown alleles.

Immunization Protocol

Immune sera for FACS experiments were obtained as follows.

Groups of 4 CD-1 outbred female mice 6-7 weeks old (Charles River Laboratories, Calco Italy) were immunized with the selected GBS antigens, (20 μg of each recombinant GBS antigen), suspended in 100 μl of PBS. Each group received 3 doses at days 0, 21 and 35. Immunization was performed through intra-peritoneal injection of the protein with an equal volume of Complete Freund's Adjuvant (CFA) for the first dose and Incomplete Freund's Adjuvant (IFA) for the following two doses. In each immunization scheme negative and positive control groups are used. Immune response was monitored by using serum samples taken on day 0 and 49.

FACS Analysis

Preparation of paraformaldehyde treated GBS cells and their FACS analysis were carried out as follows.

GBS serotype COH1 strain cells were grown in Todd Hewitt Broth (THB; Difco Laboratories, Detroit, Mich.) to OD600 nm=0.5. The culture was centrifuged for 20 minutes at 5000 rpm and bacteria were washed once with PBS, resuspended in PBS containing 0.05% paraformaldehyde, and incubated for 1 hours at 37 ° C. and then overnight at 4° C. 50 μl of fixed bacteria (OD600 0.1) were washed once with PBS, resuspended in 20 μl of Newborn Calf Serum, (Sigma) and incubated for 20 min. at room temperature. The cells were then incubated for 1 hour at 4° C. in 100 μl of preimmune or immune sera, diluted 1:200 in dilution buffer (PBS, 20% Newborn Calf Serum, 0.1% BSA). After centrifugation and washing with 200 μl of washing buffer (0.1% BSA in PBS), samples were incubated for 1 hour at 4° C. with 50 μl of R-Phicoerytrin conjugated F(ab)2 goat anti-mouse IgG (Jackson ImmunoResearch Laboratories; Inc.), diluted 1:100 in dilution buffer. Cells were washed with 200 μl of washing buffer and resuspended in 200 μl of PBS. Samples were analysed using a FACS Calibur apparatus (Becton Dickinson, Mountain View, Calif.) and data were analyzed using the Cell Quest Software (Becton Dickinson). A shift in mean fluorescence intensity of >75 channels compared to preimmune sera from the same mice was considered positive. This cutoff was determined from the mean plus two standard deviations of shifts obtained with control sera raised against mock purified recombinant proteins from cultures of E. coli carrying the empty expression vector and included in every experiment. Artifacts due to bacterial lysis were excluded using antisera raised against 6 different known cytoplasmic proteins all of which were negative

FACS data on COH1 single KO mutants for GBS 104 and GBS 80 indicated that GBS 80 is required for surface localization of GBS 104.

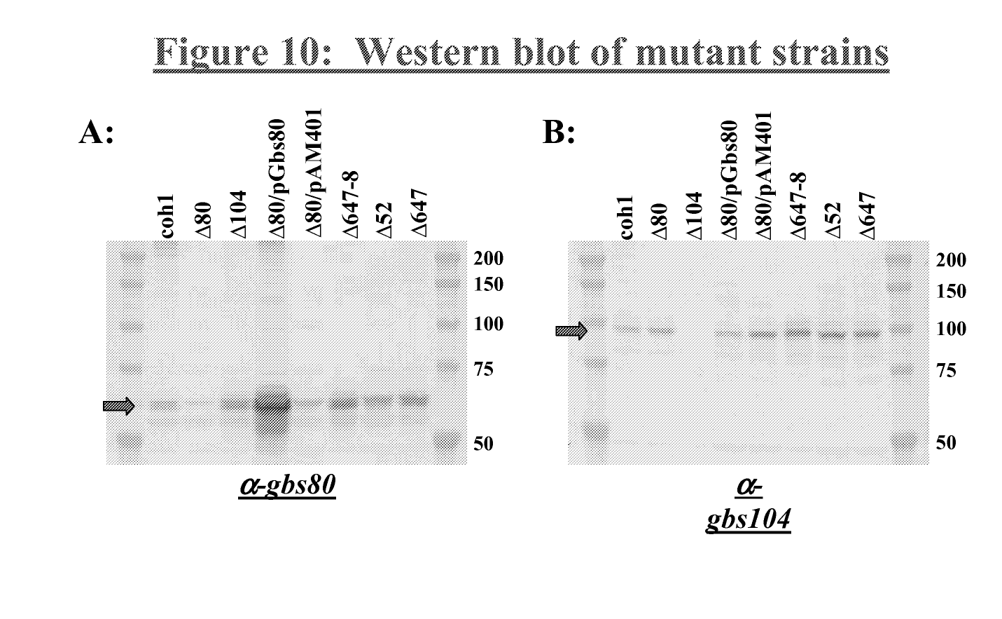

As shown in FIG. 8, GBS 104 is not surface exposed in the Δ80 strain (second column, bottom), but is present in the whole protein extracts (see FIG. 10). Mean shift values suggest that GBS 104 is partially responsible for GBS 80 surface exposure (Mean shift of GBS 80 is reduced to ˜60% wild-type levels in Δ104), and that GBS 80 is over-expressed in the complemented strain (mean shift value ˜200% wild-type level). The Δ80/pGBS 80 strain contains the GBS 80 orf cloned in the shuttle-vector pAM401 (Wirth, R., F. Y. An, et al. (1986). J Bacteriol 165(3): 831-6). The vector alone does not alter the secretion pattern of GBS 104 (right column) FACS was performed on mid-log fixed bacteria with mouse polyclonal antibodies as indicated at left. Black peak is pre-immune sera, colored peaks are sera from immunized animals.

### Example 3

### Deletion of GBS 80 Causes Attenuation In Vivo

This example demonstrates that deletion of GBS 80 causes attenuation in vivo, suggesting that this protein contributes to bacterial virulence.

By using a mouse animal model, we studied the role of GBS 80 and GBS 104 in the virulence of S. agalactiae.

Groups of ten outbred female mice 5-6 week weeks old (Charles River Laboratories, Calco Italy) were inoculated intraperitoneally with different dilutions of the mutant strains and LD50 (lethal dose 50) were calculated according to the method of Reed and Muench [Reed, L. J. and H. Muench (1938). The American Journal of Hygiene 27(3): 493-7]. As presented in Table 3 below the number of colony forming units (cfu) counted for both the Δ80 and the Δ80, Δ104 double mutants is about 10 fold higher when compared to the wild type strain suggesting that inactivation of GBS 80 but not GBS 104 is responsible for an attenuation in virulence. This finding indicates that GBS 80 gene in the AI-1 might contribute to virulence.

TABLE 3
Lethal dose 50% analysis of AI-1 mutants
in the 2603 strain background.

| GBS strain | LD₅₀, cfu | Number of Experiments |
|---|---|---|
| Wild Type 2603 | 2 × 10⁸ | 4 |
| Δ104 mutant | ~2 × 10⁸ | 1 |
| Δ80 mutant | 2.6 × 10⁹ | 3 |
| Δ80, Δ104 double mutant | ~2 × 10⁹ | 1 |

LD₅₀s were performed by IP injection of female CD1 mice at an age of 5-6 weeks. LD₅₀s were calculated by the method of Reed and Muench (8).

### Example 4

### Effect of Adhesin Island Sortase Deletions on Surface Antigen Presentation

This example demonstrates the effect of adhesin island sortase deletions on surface antigen presentation.

FACS analysis results set forth in FIG. 9 show that a deletion in sortase SAG0648 prevented GBS 104 from reaching the surface and slightly reduced the surface exposure of GBS 80 (fourth panel; mean shift value ˜60% wild-type COH1). In the double sortase knock-out strain, neither antigen was surface exposed (far right panel). Either sortase alone was sufficient for GBS 80 to arrive at the bacterial surface (third and fourth columns, top). No effect was seen on surface exposure of antigens GBS 80 or GBS 104 in the ΔGBS 52 strain.

Antibodies derived from purified GBS 52 were either non-specific or were FACS negative for GBS 52. FACS analysis was performed as described above (see EXAMPLE 2).

As shown in FIG. 10, inactivation of GBS 80 has no effect on GBS 104 expression as much as GBS 104 knock out doesn't change the total amount GBS 80 expressed. The Western blot of whole protein extracts (strains noted above lanes) probed with anti-GBS 80 antisera is shown in panel A. Arrow indicates expected size of GBS 80 (60 kDa). GBS 80 antibodies recognize a doublet, the lower band is not present in ΔGBS 80 strains. Panel B shows a Western blot of whole protein extracts probed with anti-GBS 104 antisera. Arrow indicates expected size of GBS 104 (99.4 kDa). Protein extracts were prepared from the same bacterial cultures used for FACS (FIGS. 8 and 9). In conclusion, although GBS 104 does not arrive at the surface in the Δ80 strain by FACS (FIG. 8, second column), it is present at approximately wild-type levels in the whole protein preps (B, second lane). Approximately 20 μg of each protein extract was loaded per lane.

Western-Blot Analysis

Aliquots of total protein extract mixed with SDS loading buffer (1×: 60 mM TRIS-HCl pH 6.8, 5% w/v SDS, 10% v/v glycerin, 0.1% Bromophenol Blue, 100 mM DTT) and boiled 5 minutes at 95° C., were loaded on a 12.5% SDS-PAGE precast gel (Biorad). The gel is run using a SDS-PAGE running buffer containing 250 mM TRIS, 2.5 mM Glycine and 0.1% SDS. The gel is electroblotted onto nitrocellulose membrane at 200 mA for 60 minutes. The membrane is blocked for 60 minutes with PBS/0.05% Tween-20 (Sigma), 10% skimmed milk powder and incubated O/N at 4° C. with PBS/0.05% Tween 20, 1% skimmed milk powder, with the appropriate dilution of the sera. After washing twice with PBS/0.05% Tween, the membrane is incubated for 2 hours with peroxidase-conjugated secondary anti-mouse antibody (Amersham) diluted 1:4000. The nitrocellulose is washed three times for 10 minutes with PBS/0.05% Tween and once with PBS and thereafter developed by Opti-4CN Substrate Kit (Biorad).

### Example 5

### Binding of Adhesin Island Proteins to Epithelial Cells and Effect of Adhesin Island Proteins on Capacity of GBS to Adhere to Epithelial Cells

This example illustrates the binding of AI proteins to epithelial cells and the effect of AI proteins on the capacity of GBS to adhere to epithelial cells.

Applicants analysed whether recombinant AI surface proteins GBS 80 or GBS 104 would demonstrate binding to various epithelial cells in a FACS analysis. Applicants also analysed whether deletion of AI surface proteins GBS 80 or GBS 104 would effect the capacity of GBS to adhere to and invade ME180 cervical epithelial cells.

As shown in FIG. 28, deletion of GBS 80 sequence from GBS strain isolate 2603 (serotype V) did not affect the capacity of the mutated GBS to adhere to and invade ME180 cervical epithelial cells. Here ME180 cervical carcinoma epithelial cells were infected with wild type GBS 2603 or GBS 2603 Δ80 isogenic mutant. After two hours of infection, non-adherent bacteria were washed off and infection prolonged for a further two hours and four hours. In invasion experiments, after each time point, was followed by a two hour antibiotic treatment. Cells were then lysed with 1% saponin and lysates platedon TSA plates. As shown in FIG. 28, there was little difference between the percent invasion or percent adhesion of wild type and mutant strains up to the four hour time point.

FIG. 30 repeats this experiment with both Δ104 and Δ80 mutants from a different strain isolate. Here, ME180 cervical carcinoma epithelial cells were infected with GBS strain isolate COH (serotype III) wild type or COH1 ΔGBS 104 or COH1 Δ80 isogenic mutant. After one hour of infection, non-adherent bacteria were washed off and the cells were lysed with 1% saponin. The lysates were plated on TSA plates. As shown in FIG. 30, while there was little difference in the percent invasion, there was a significant decrease in the percent association of the Δ104 mutant compared to both the wild type and Δ80 mutant.

The affect of AI surface proteins on the ability of GBS to translocate through an epithelial monolayer was also analysed. As shown in FIG. 31, a GBS 80 knockout mutant strain partially loses the ability to translocate through an epithelial monolayer. Here epithelial monolayers were inoculated with wildtype or knockout mutant in the apical chamber of a transwell system for two hours and then non-adherent bacteria were washed off. Infection was prolonged for a further two and four hours. Samples were taken from the media of the basolateral side and the number of colony forming unties measured. Transepithelial electrical resistance measured prior to and after infection gave comparable values, indicating the maintenance of the integrity of the monolayer. By the six hour time point, the Δ80 mutants demonstrated a reduced percent transcytosis.

A similar experiment was conducted with GBS 104 knock out mutants. Here, as shown in FIG. 22, the Δ104 mutants also demonstrated a reduced percent transcytosis, indicating that the mutant strains translocate through an epithelial monolayer less efficiently than their isogenic wild type counterparts.

Applicants also studied the effect of AI proteins on the capacity of a GBS strain to invade J774 macrophage-like cells. Here, J774 cells were infected with GBS COH1 wild type or COH1 ΔGBS104 or COH1 ΔGBS80 isogenic mutants. After one hour of infection, non-adherent bacteria were washed off and intracellular bacteria were recovered at two, four and six hours post antibiotic treatment. At each time point, cells were lysed with 0.25% Triton X-100 and lysates plated on TSA plates. As shown in FIG. 32, the Δ104 mutant demonstrated a significantly reduced percent invasion compared to both the wild type and Δ80 mutant.

### Example 6

### Hyperoligomeric Structures Comprising AI Surface Proteins GBS 80 and GBS 104

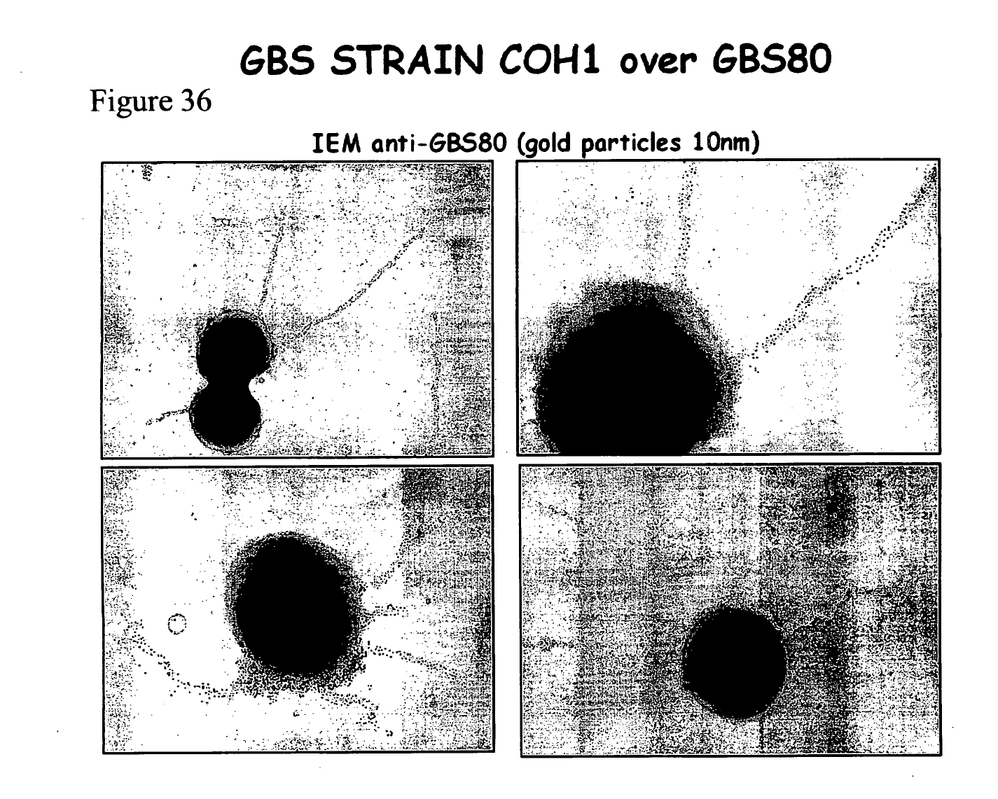

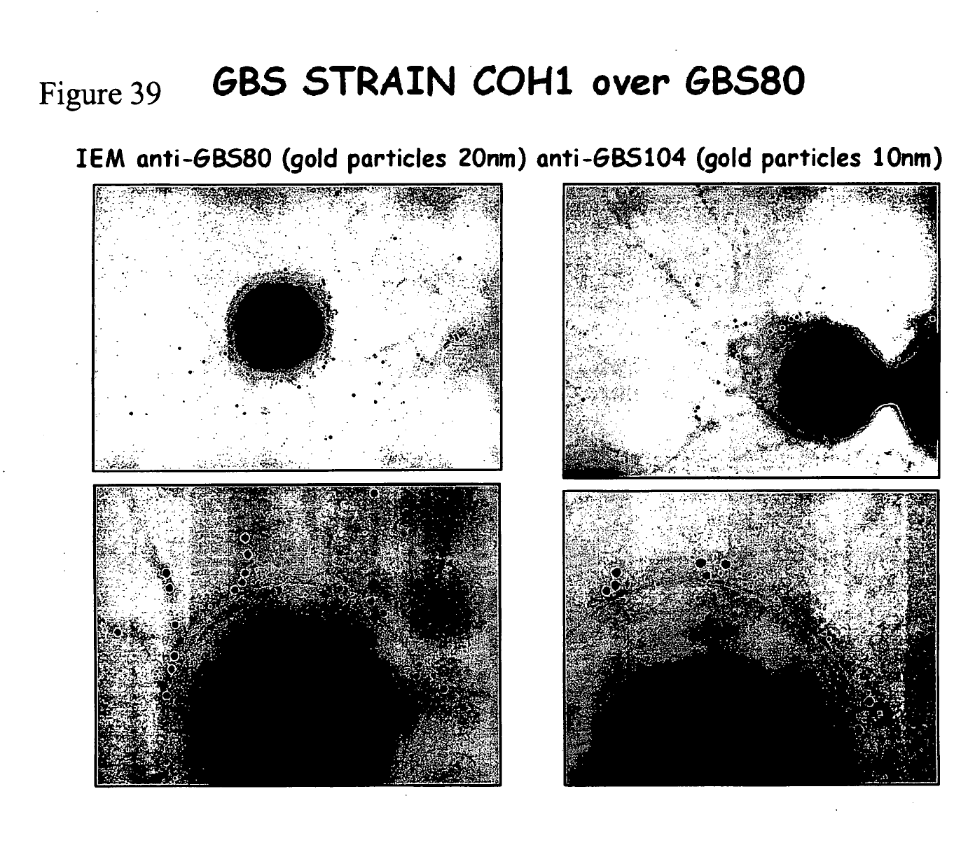

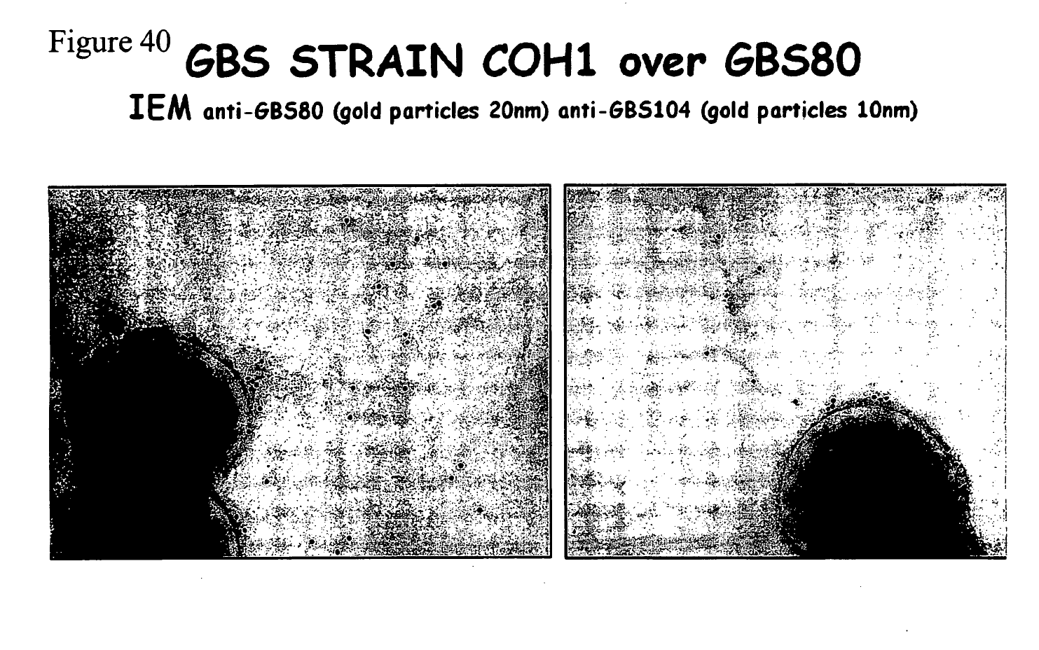

This example illustrates hyperoligomeric structures comprising AI surface proteins GBS 80 and GBS 104. A GBS isolate COH1 (serotype III) was adapted to increase expression of GBS 80. FIG. 34 presents a regular negative stain electron micrograph of this mutant; no pilus or hyperoligomeric structures are distinguishable on the surface of the bacteria. When the EM stain is based on anti-GBS 80 antibodies labelled with 10 or 20 nm gold particles, the presence of GBS 80 throughout the hyperoligomeric structure is clearly indicated (FIGS. 36, 37 and 38). EM staining against GBS 104 (anti-GBS 104 antibodies labelled with 10 nm gold particles) also reveals the presence of GBS 104 primarily on or near the surface of the bacteria or potentially associated with bacterial peptidoglycans (FIG. 39). Analysis of this same strain (over-expressing GBS 80) with a combination of both anti-GBS 80 (using 20 nm gold particles) and anti-GBS 104 (using 10 nm gold particles) reveals the presence of GBS 104 on the surface and within the hyperoligomeric structures (see FIGS. 40 and 41).

### Example 7

### GBS 80 is Necessary for Polymer Formation and GBS 104 and Sortase SAG0648 are Necessary for Efficient Pili Assembly

This example demonstrates that GBS 80 is necessary for formation of polymers and that GBS 104 and sortase SAG0648 are necessary for efficient pili assembly. GBS 80 and GBS 104 polymeric assembly was systematically analyzed in Coh1 strain single knock out mutants of each of the relevant coding genes in AI-1 (GBS 80, GBS 104, GBS 52, sag0647, and sag0648). FIG. 41 provides Western blots of total protein extracts (strains noted above lanes) probed with either anti-GBS 80 (left panel) sera or anti-GBS 104 sera (right panel) for each of these Coh1 and Coh1 knock out strains. (Coh1, wild type Coh1; Δ80, Coh1 with GBS 80 knocked out; Δ104, Coh1 with GBS 104 knocked out; Δ52, Coh1 with GBS 52 knocked out; Δ647, Coh1 with SAG0647 knocked out; Δ648, Coh1 with SAG0648 knocked out, Δ647-8, Coh1 with SAG0647 and SAG0648 knocked out; Δ80/pGBS80, Coh1 with GBS 80 knocked out but complemented with a high copy number plasmid expressing GBS 80. Asterisks identify the monomer of GBS 80 and GBS 104.)

The smear of immunoreactive material observed in the wild type strain, along with its disappearance in Δ80 and Δ104 mutants, is consistent with the notion that such high molecular weight structures are composed of covalently linked (SDS-resistant) GBS 80 and GBS 104 subunits. The immunoblotting with both anti-GBS 80 (α-GBS 80) and anti-GBS 104 (α-GBS 104) revealed that deletion of sortase SAG0648 also interferes with the assembly of high molecular weight species, whereas the knock out mutant of the second sortase (SAG0647), even if somehow reduced, still maintains the ability to form polymeric structures.

Total extracts form GBS were prepared as follows. Bacteria were grown in 50 ml of Todd-Hewitt broth (Difco) to an OD600 nm of 0.5-0.6 and successively pelleted. After two washes in PBS the pellet was resuspended and incubated 3 hours at 37° C. with mutanolisin. Cells were then lysed with at least three freezing-thawing cycles in dry ice and a 37° C. bath. The lysate was then centrifuged to eliminate the cellular debris and the supernatant was quantified. Approximately 40 μg of each protein extract was separated on SDS-PAGE. The gel was then subjected to immunoblotting with mice antisera and detected with chemiluminescence.

### Example 8

### GBS 80 is Polymerized by an AI-2 Sortase

This example illustrates that GBS 80 can be polymerized not only by AI-1 sortases, but also by AI-2 sortases. FIG. 42 shows total cell extract immunoblots of GBS 515 strain, which lacks AI-1. The left panel, where an anti-GBS 67 sera was used, shows that GBS 67 from AI-2 is assembled into high-molecular weight-complexes, suggesting the formation of a second type of pilus. The same high molecular structure is observed when GBS 80 is highly expressed by reintroducing the gene within a plasmid (pGBS 80). By using anti-GBS 80 (right panel) sera on the same extracts, again it is observed that, with GBS 80 over expression (515/pGBS 80), a high-molecular weight structure is assembled. This implies that, in the absence of AI-1 sortases, AI-2 sortases (SAG1405 and SAG1406) can complement the lacking function, still being able to assemble GBS 80 in a pilus structure.

### Example 9

### Coh1 Produces a High Molecular Weight Molecule, the GBS 80 Pilin

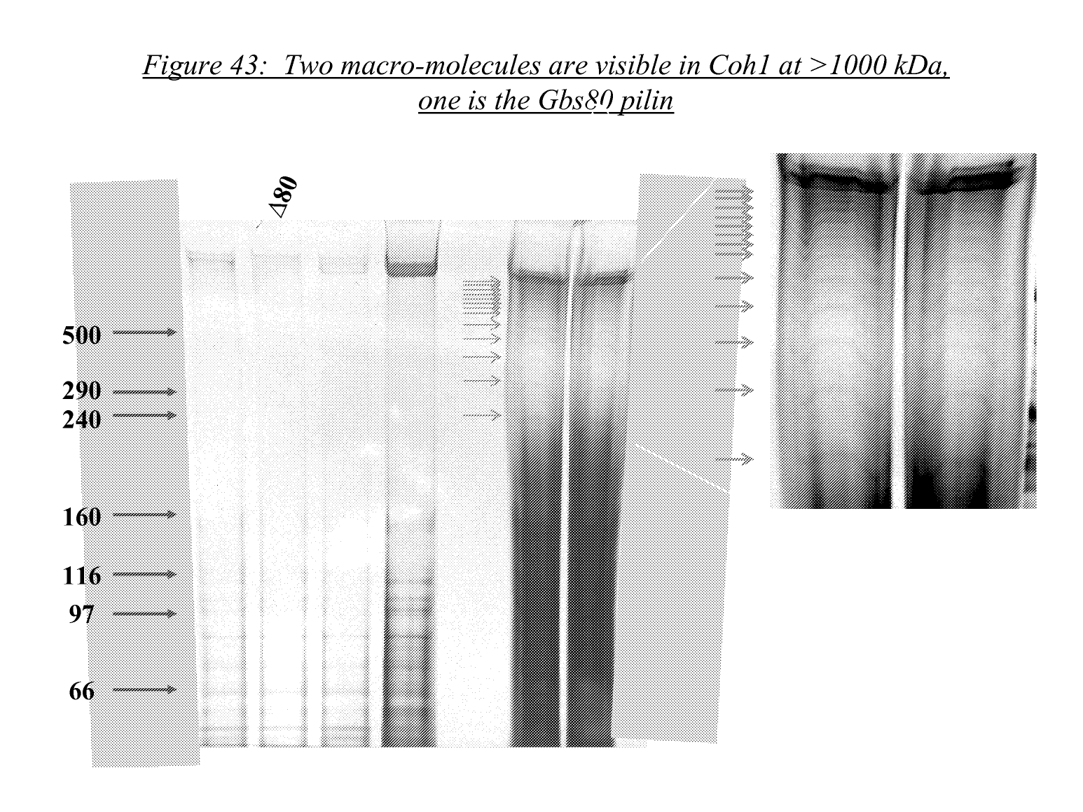

This example illustrates that Coh1 produces a high molecular weight molecule, greater than 1000 kDa, which is the GBS 80 pilin. FIG. 43 provides silver-stained electrophoretic gels that show that Coh1 produces two macromolecules. One of these macromolecules disappears in the Coh1 GBS 80 knock out cells, but does not disappear in the Coh1 GBS 52 knock out mutant cells. The last two lanes on the right were loaded with 15 times the amount loaded in the other lanes. This was done in order to be able to count the bands. By doing this, a conservative size estimate of the top bands was calculated by starting at 240 kDa and considering each of 14 higher bands as the result of consecutive additions of a GBS 80 monomer.

Coh1, wild type Coh1; Δ80, Coh1 cells with GBS 80 knocked out; Δ52, Coh1 cells with GBS 52 knocked out; Δ80/pGBS 80, Coh1 cells with GBS 80 knocked out and complemented with a high copy number construct expressing GBS 80.

### Example 10

### GBS 52 is a Minor Component of the GBS Pilus

This example illustrates that GBS 52 is present in the GBS pilus and is a minor component of the pilus. FIG. 45 shows an immunoblot of total cell extracts from a GBS Coh1 strain and a GBS Coh1 strain knocked out for GBS 52 (Δ52). The total cell extracts were immunoblotted anti-GBS 80 antisera (left) and anti-GBS 52 antisera (right). Immunoblotting was performed using a 3-8% Tris-acetate polyacrylamide gel (Invitrogen) which provided excellent separation of large molecular weight proteins (see FIG. 41). When the gel was incubated with anti-GBS 80 sera, the bands from the Coh1 wild-type strain appeared shifted when compared to the Δ52 mutant. This observation indicated a different size of the pilus polymeric components in the two strains. When the same gel was stripped and incubated with anti-GBS 52 sera the high-molecular subunits in the Coh1 wild-type strain showed similar molecular size of those in the correspondent lane in the left panel. These findings confirmed that GBS 52 is indeed associated with GBS 80 macro-molecular structures but represents a minor component of the GBS pilus.

### Example 11

### Pilus Structures are Present in the Supernatant of GBS Bacterial Cultures

This example illustrates that the pilus structure assembled in Coh1 GBS is present in the supernatant of a bacterial cell culture. FIG. 46 shows an immunoblot where the protein extract of the supernatant from cultures of different GBS mutant strains (117=Coh1 GBS 80 knockout; 159=Coh1 GBS 104 knockout; 202=Coh1 GBS 52 knockout; 206=Coh1 GBS sag0647 knockout; 208=Coh1 GBS sag0648 knockout; 197=Coh1 GBS sag0647/sag0648 knockout; 179=Coh1 GBS 80 knockout complemented with a high copy plasmid expressing GBS 80). GBS 80 antisera detects the presence of pilus structures in the appropriate Coh1 strains.

The protein extract was prepared as follows. Bacteria were grown in THB to an OD_{600 nm} of 0.5-0.6 and the supernatant was separated from the cells by centrifugation. The supernatant was then filtered (Ø0.2 μm) and 1 ml was added with 60% TCA for protein precipitation.

GBS pili were also extracted from the fraction of surface-exposed proteins in Coh1 strain and its GBS 80 knock out mutant as described hereafter. Bacteria were grown to an OD_{600 nm} of 0.6 in 50 ml of THB at 37° C. Cells were washed once with PBS and the pellet was then resuspended in 0.1 M KPO4 pH 6.2, 40% sucrose, 10 mM MgCl2, 400 U/ml mutanolysin and incubated 3 hours at 37° C. Protoplasts were separated by centrifugation and the supernatant was recovered and its protein content measured.

In order to study the dynamics of pilus production during different growth phases, 1 ml supernatant of a culture at different OD_{600 nm} was TCA precipitated and loaded onto a 3-8% SDS-PAGE as described before. FIG. 47 shows the corresponding Western blot with GBS 80 anti-sera. The first group of lanes (left five sample lanes) refer to a Coh1 strain growth (OD_{600 nm} are noted above the lanes) whereas the second group of lanes (right five samples) are from a GBS 80 knock out strain over expressing GBS 80. The experiment shows that pilus macromolecular structures can be found in the supernatant in all of the growth phases tested.

### Example 12

### In GBS Strain Coh1, Only GBS 80 and a Sortase (sag0647 or sag0648) is Required for Polymerization

This example describes requirements for pilus formation in Coh1. FIG. 48 shows a Western blot of total protein extracts (prepared as described before) using anti-GBS 80 sera on Coh1 clones. (Coh1, wild type Coh1; Δ104, Coh1 knocked out for GBS 104, Δ647, Coh1 knocked out for sag0647, Δ648, Coh1 knocked for sag0648, Δ647-8, Coh1 knocked out for sag0647 and sag0648; 515, wild type bacterial strain 515, which lacks an AI-1; p80 a high copy number plasmid which expresses GBS 80.) The data show that only the double sortase mutant is unable to polymerize GBS 80 indicating that the ‘conditio sine qua non’ for pilus polymerization is the co-existence of GBS 80 with at least one sortase. This result leads to a reasonable assumption that SAG1405 and SAG1406 are responsible for polymerization in this strain.

### Example 13

### GBS 80 Can be Expressed in L. lactis Under its Own Promoter and Terminator Sequences

This example demonstrates that L. lactis, a non-pathogenic bacterium, can express GBS AI polypeptides such as GBS 80. L. lactis M1363 (J. Bacteriol. 154 (1983):1-9) was transformed with a construct encoding GBS 80. Briefly, the construct was prepared by cloning a DNA fragment containing the gene coding for GBS 80 under its own promoter and terminator sequences into plasmid pAM401 (a shuttle vector for E. coli and other Gram positive bacteria; J. Bacteriol. 163 (1986):831-836). Total extracts of the transformed bacteria in log phase were separated on SDS-PAGE, transferred to membranes, and incubated with antiserum against GBS 80. A polypeptide corresponding to the molecular weight of GBS 80 was detected in the lanes containing total extracts of L. lactis transformed with the GBS 80 construct. See FIGS. 133A and 133B, lanes 6 and 7. This same polypeptide was not detected in the lane containing total extracts of L. lactis not transformed with the GBS 80 construct, lane 9. This example shows that L. lactis can express GBS 80 under its own promoter and terminator.

### Example 14

### L. lactis Modified to Express GBS AI-1 Under the GBS 80 Promoter and Terminator Sequences Expresses GBS 80 in Polymeric Structures

This example demonstrates the ability of L. lactis to express GBS AI-1 polypeptides and to incorporate at least some of the polypeptides into oligomers. L. lactis was transformed with a construct containing the genes encoding GBS AI-1 polypeptides. Briefly, the construct was prepared by cloning a DNA fragment containing the genes for GBS 80, GBS 52, SAG0647, SAG0648, and GBS 104 under the GBS 80 promoter and terminator sequences into construct pAM401. The construct was transformed into L. lactis M1363. Total extracts of log phase transformed bacteria were separated on reducing SDS-PAGE, transferred to membranes, and incubated with antiserum against GBS 80. A polypeptide with a molecular weight corresponding to the molecular weight of GBS 80 was detected in the lanes containing L. lactis transformed with the GBS AI-1 encoding construct. See FIG. 134, lane 2. In addition, the same lane also showed immunoreactivity of polypeptides having higher molecular weights than the polypeptide having the molecular weight of GBS 80. These higher molecular weight polypeptides are likely oligomers of GBS 80. Oligomers of similar molecular weights were also observed on a Western blot of the culture supernatant of the transformed L. lactis. See lane 4 of FIG. 135. Thus, this example shows that L. lactis transformed to express GBS AI-1 can efficiently polymerize GBS 80 in the form of a pilus. This pilus structure can likely be purified from either the cell culture supernatant or cell extracts.

### Example 15

### Cloning and Expression of S. pneumoniae Sp0462

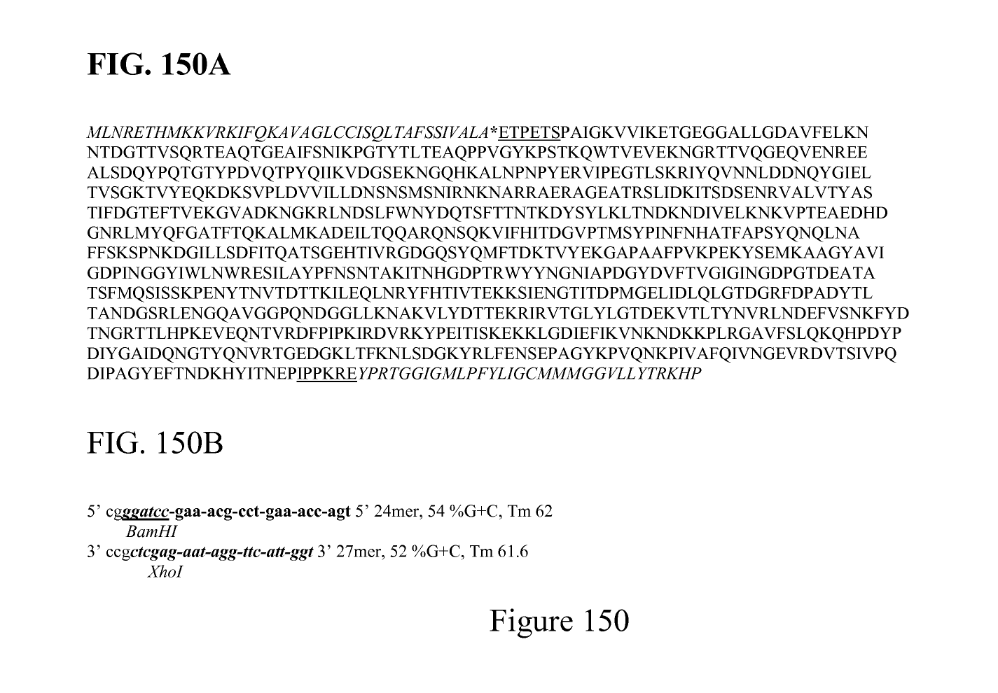

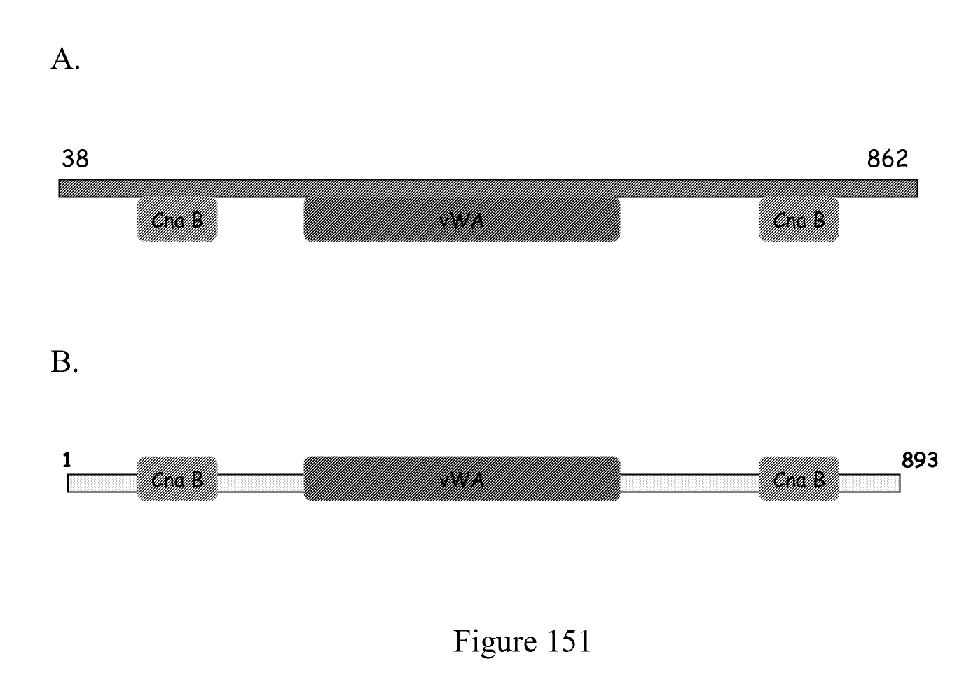

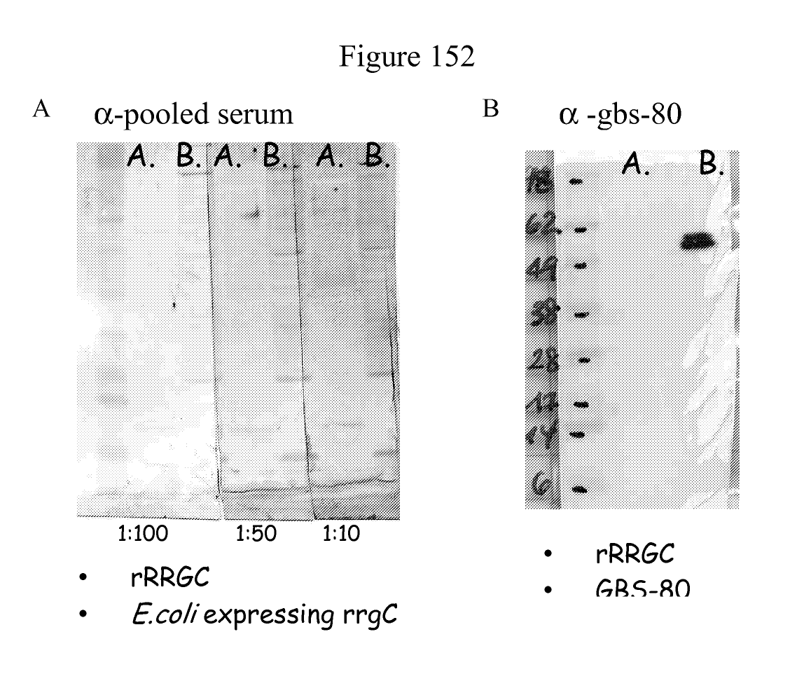

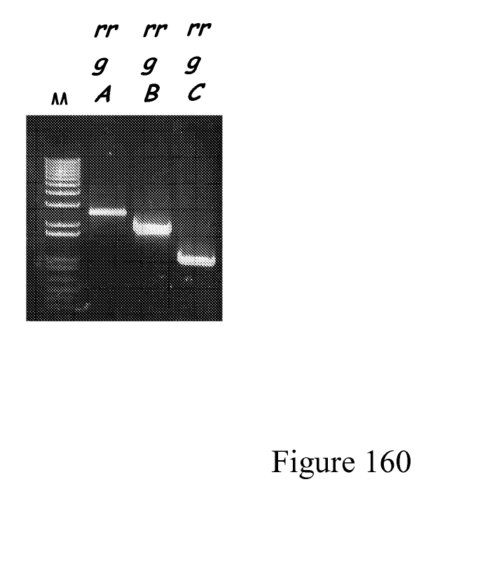

This example describes the production of a clone encoding a Sp0462 polypeptide and expression of the clone. To produce a clone encoding Sp0462, the open reading frame encoding Sp0462 was amplified using primers that annealed within the full-length Sp0462 open reading frame sequence. FIG. 150A provides a 893 amino acid sequence of Sp0462. The primers used to produce a clone encoding the Sp0462 polypeptide are shown in FIG. 150B. These primers annealed to the nucleotide sequences encoding the amino acid residues indicated by underlining in FIG. 150A. Amplification of the open reading frame encoding Sp0462 using these primers produced the amplicon shown at lane 2 of the agarose gel provided in FIG. 160. The Sp0462 clone encodes amino acid residues 38-862 of the 893 amino acid residue Sp0462 protein; the italicized residues in FIG. 150A were eliminated. FIG. 151A provides a schematic depiction of the recombinant Sp0462 polypeptide. FIG. 151B shows a schematic depiction of the full-length Sp0462 polypeptide. Both the recombinant Sp0462 encoded by the clone and the full-length Sp0462 protein have two collagen binding protein type B (Cna B) domains and a von Hillebrand factor A (vWA) domain. The cloned recombinant Sp0462 lacks the LPXTG motif present in the full-length Sp0462 protein. Western blot analysis for expression of the Sp0462 clone did not result in detection of polypeptides with serum obtained from S. pneumoniae-infected patients (FIG. 152A) or GBS 80 antiserum (FIG. 152B).

### Example 16

### Cloning and Expression of S. pneumoniae Sp0463

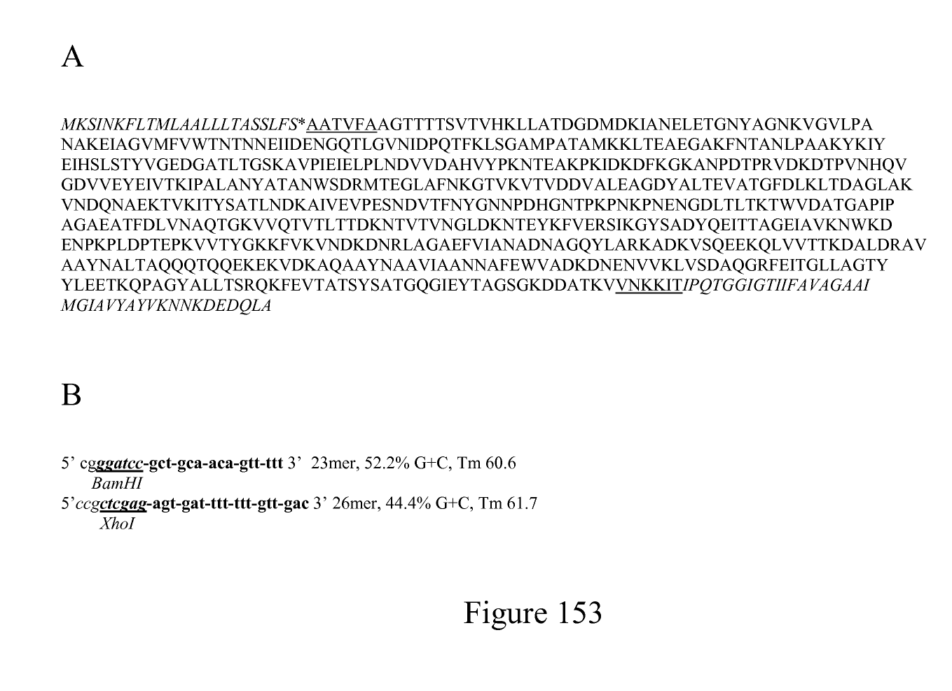

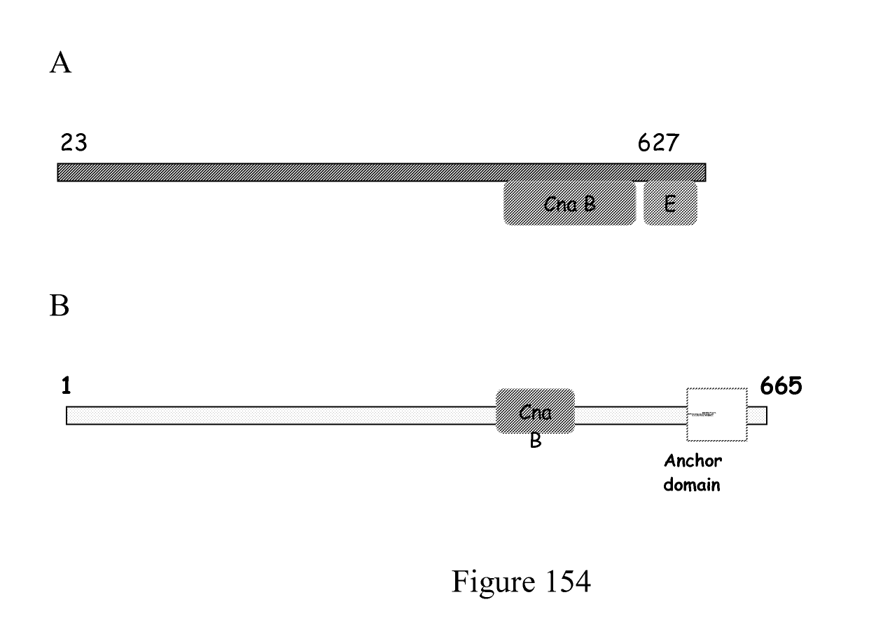

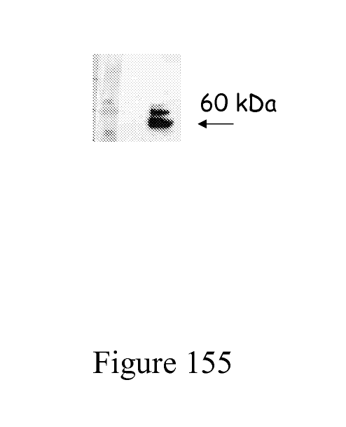

This example describes the production of a clone encoding a Sp0463 polypeptide and detection of recombinant Sp0463 polypeptide expressed from the clone. To produce a clone encoding Sp0463, the open reading frame encoding Sp0463 was amplified using primers that annealed within the full-length Sp0463 open reading frame sequence. FIG. 153A provides a 665 amino acid sequence of Sp0463. The primers used to produce the clone encoding Sp0463 polypeptide are shown in FIG. 153B. These primers annealed to the nucleotide sequences encoding the amino acid residues indicated by underlining in FIG. 153A. Amplification of the open reading frame encoding Sp0463 using these primers produced the amplicon shown at lane 3 of the agarose gel provided in FIG. 160. The Sp0463 clone encodes amino acid residues 23-627 of the 665 amino acid residue Sp0463 protein; the italicized residues in FIG. 153A were eliminated. FIG. 154A provides a schematic depiction of the recombinant Sp0463 polypeptide. FIG. 154B shows a schematic depiction of the full-length Sp0463 polypeptide. Both the recombinant Sp0463 encoded by the clone and the full-length Sp0463 protein have a Cna B domain and an E box motif. The cloned recombinant Sp0463 lacks the LPXTG motif present in the full-length Sp0463 protein. Expression of the Sp0463 clone resulted in the detection of a 60 kD polypeptide, the expected molecular weight of the recombinant Sp0463 polypeptide, by Western blot analysis. See FIG. 155.

### Example 17

### Cloning and Expression of S. pneumoniae Sp0464

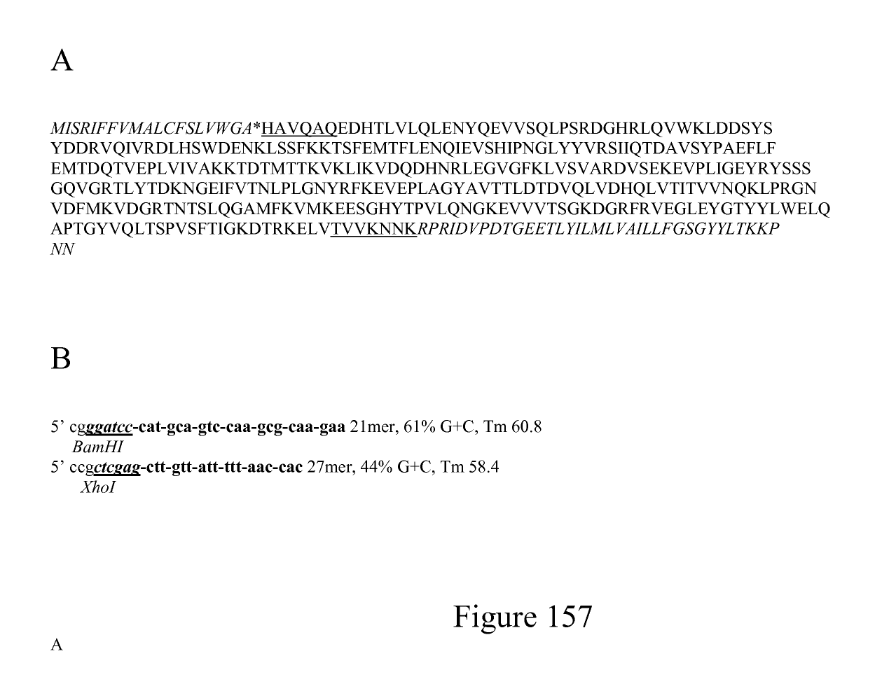

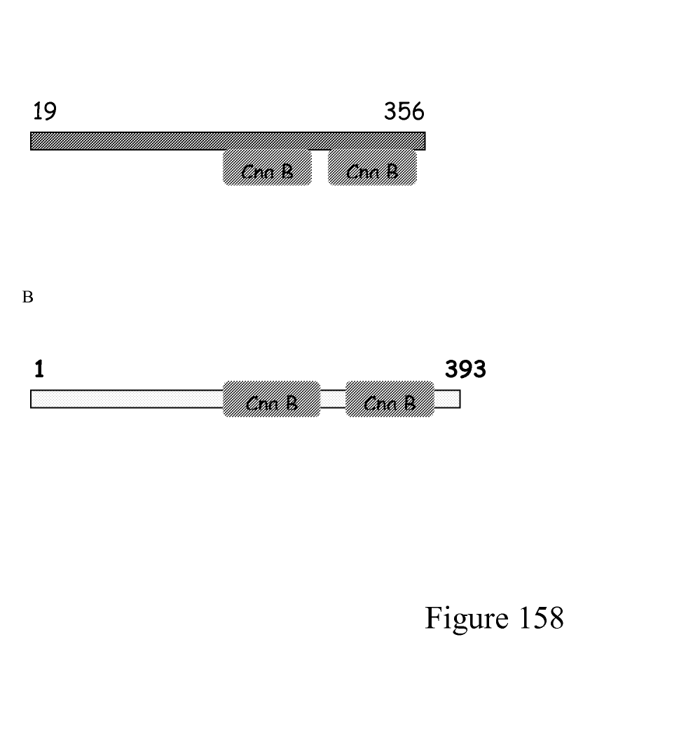

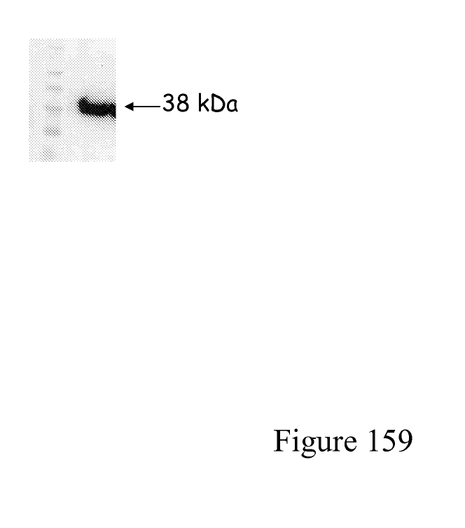

This example describes the production of a clone encoding a Sp0464 polypeptide and detection of recombinant Sp0464 polypeptide expressed from the clone. To produce a clone encoding Sp0464, the open reading frame encoding Sp0464 was amplified using primers that annealed either within the full-length Sp0464 open reading frame sequence. FIG. 157A provides a 393 amino acid sequence of Sp0464. The primers used to produce a clone encoding the Sp0464 polypeptide are shown in FIG. 157B. These primers annealed to the nucleotide sequences encoding the amino acid residues indicated by underlining in FIG. 157A. Amplification of the open reading frame encoding Sp0464 using these primers produced the amplicon shown at lane 4 of the agarose gel provided in FIG. 160. The Sp0464 clone encodes amino acid residues 19-356 of the 393 amino acid residue Sp0464 protein; the italicized residues in FIG. 157A were eliminated. FIG. 158A provides a schematic depiction of the recombinant Sp0464 polypeptide. FIG. 158B shows a schematic depiction of the full-length Sp0464 polypeptide. Both the recombinant Sp0464 encoded by the clone and the full-length Sp0464 protein have two Cna B domains. The cloned recombinant Sp0464 lacks the LPXTG motif present in the full-length Sp0464 protein. Expression of the Sp0464 clone resulted in the detection of a 38 kD polypeptide, the expected molecular weight of the recombinant Sp0464 polypeptide, by Western blot analysis. See FIG. 159.

### Example 18

### Intranasal Immunization of Mice With Recombinant L. lactis Expressing GBS 80 and Subsequent Challenge

This example describes a method of intranasally immunizing mice using L. lactis that express GBS 80. Intranasal immunization consisted of 3 doses at days 0, 14 and 28, each dose administered in three consecutive days. Each day, groups of 3 CD-1 outbred female mice 6-7 weeks old (Charles River Laboratories, Calco Italy) were immunized intranasally with 10⁹ or 10¹⁰ CFU of the recombinant Lactococcus lactis suspended in 20 μl of PBS. In each immunization scheme negative (wild-type L. lactis) and positive (recombinant GBS80) control groups were used. The immune response of the dams was monitored by using serum samples taken on day 0 and 49. The female mice were bred 2-7 days after the last immunization (at approximately t=36-37), and typically had a gestation period of 21 days. Within 48 hours of birth, the pups were challenged via I.P. with GBS in a dose approximately equal to an amount which would be sufficient to kill 90% of immunized pups (as determined by empirical data gathered from PBS control groups). The GBS challenge dose is preferably administered in 50 ml of THB medium. Preferably, the pup challenge takes place at 56 to 61 days after the first immunization. The challenge inocula were prepared starting from frozen cultures diluted to the appropriate concentration with THB prior to use. Survival of pups was monitored for 5 days after challenge.

### Example 19

### Subcutaneous Immunization of Mice With Recombinant L. lactis Expressing GBS 80 and Subsequent Challenge

This example describes a method of subcutaneous immunization mice using L. lactis that express GBS 80. Subcutaneous immunization consists of 3 doses at days 0, 14 and 28. Groups of 3 CD-1 outbred female mice 6-7 weeks old (Charles River Laboratories, Calco Italy) were injected subcutaneously with 10⁹ or 10¹⁰ CFU of the recombinant Lactococcus lactis suspended in 100 μl of PBS. In each immunization scheme, negative (wild-type L. lactis) and positive (recombinant GBS80) control groups were used. The immune response of the dams was monitored by using serum samples taken on day 0 and 49. The female mice were bred 2-7 days after the last immunization (at approximately t=36-37), and typically had a gestation period of 21 days. Within 48 hours of birth, the pups were challenged via I.P. with GBS in a dose approximately equal to an amount which would be sufficient to kill 90% of immunized pups (as determined by empirical data gathered from PBS control groups). The GBS challenge dose is preferably administered in 50 ml of THB medium. Preferably, the pup challenge takes place at 56 to 61 days after the first immunization. The challenge inocula were prepared starting from frozen cultures diluted to the appropriate concentration with THB prior to use. Survival of pups was monitored for 5 days after challenge.

### Example 20

### Immunization of Mice With GAS AI Polypeptides and Subsequent Intranasal Challenge

This example describes a method of immunizing mice with GAS AI polypeptides and subsequently intranasally challenging the mice with GAS bacteria. Groups of 10 CD1 female mice aged between 6 and 7 weeks are immunized with a combination of GAS antigens of the invention GAS 15, GAS 16, and GAS 18, (15 μg of each recombinant antigen, derived from M1 strain SF370) or L. lactis expressing the M1 strain SF370 adhesin island, suspended in 100 μl of suitable solution. Each group receives 3 doses at days 0, 21 and 45. Immunization is performed through subcutaneous or intraperitoneal injection for the GAS 15, GAS 16, GAS 18 protein combination. The protein combination is administered with an equal volume of Complete Freund's Adjuvant (CFA) for the first dose and Incomplete Freund's Adjuvant (IFA) for the following two doses. Immunization is performed intranasally for the L. lactis expressing the M1 strain SF370 adhesin island. In each immunization scheme negative and positive control groups are used.

The negative control group for the mice immunized with the GAS 15, GAS 16, GAS 18 protein combination included mice immunized with PBS. The negative control group for the mice immunized with L. lactis expressing the M1 strain SF370 adhesin island, included mice immunized with either wildtype L. lactis or L. lactis transformed with the pAM401 expression vector lacking any cloned adhesin island sequence.

The positive control groups included mice immunized with purified M1 strain SF370 M protein.

Immunized mice are then anaesthetized with Zoletil and challenged intranasally with a 25 μL suspension containing 1.2×10⁶ or 1.2×10⁸ CFU of ISS 3348 in THB Animals are observed daily and checked for survival.

### Example 21

### Active Maternal Immunization Assay

As used herein, an Active Maternal Immunization assay refers to an in vivo protection assay where female mice are immunized with the test antigen composition. The female mice are then bred and their pups are challenged with a lethal dose of GBS. Serum titers of the female mice during the immunization schedule are measured as well as the survival time of the pups after challenge.

### Mouse Immunization

Specifically, groups of 4 CD-1 outbred female mice 6-8 weeks old (Charles River Laboratories, Calco Italy) are immunized with one or more GBS antigens, (20 μg of each recombinant GBS antigen), suspended in 100 μl of PBS. Each group receives 3 doses at days 0, 21 and 35 Immunization is performed through intra-peritoneal injection of the protein with an equal volume of Complete Freund's Adjuvant (CFA) for the first dose and Incomplete Freund's Adjuvant (IFA) for the following two doses. In each immunization scheme negative and positive control groups are used.

Immune response is monitored by using serum samples taken on day 0 and 49. The sera are analyzed as pools from each group of mice.

### Active Maternal Immunization

A maternal immunization/neonatal pup challenge model of GBS infection was used to verify the protective efficacy of the antigens in mice. The mouse protection study was adapted from Rodewald et al. (Rodewald et al. J. Infect. Diseases 166, 635 (1992)). In brief, CD-1 female mice (6-8 weeks old) were immunized before breeding, as described above. The mice received 20 μg of protein per dose when immunized with a single antigen and 60 μg of protein per dose (15 μg of each antigen) when immunized with the combination of antigens. Mice were bred 2-7 days after the last immunization. Within 48 h of birth, pups were injected intraperitoneally with 50 μl of GBS culture. Challenge inocula were prepared starting from frozen cultures diluted to the appropriate concentration with THB before use. In preliminary experiments (not shown), the challenge doses per pup for each strain tested were determined to cause 90% lethality. Survival of pups was monitored for 2 days after challenge. Protection was calculated as (percentage deadControl minus percentage deadVaccine) divided by percentage deadControl multiplied by 100. Data were evaluated for statistical significance by Fisher's exact test.

### Example 22

### GBS 59 Isoforms Cross-Reactivity

In some instances GBS 59 polypeptides of different isoforms may be cross-reactive as well as GBS59 polypeptides of the same isoform may not be cross-reactive. In fact GBS 59 polypeptides are usually covalently linked in a macromolecular structure (i.e. the pilus), combined to other polypeptides such as GBS 67 and GBS 150, which show themselves some variability. Therefore the immunologic reactivity of such complex structures may not be predictable based on the sequence of single GBS 59 polypeptides. For instance in flow cytometry, where the readout is typically an average of different epitopes being recognized on these multimeric structures on the surface of the bacteria, some cross-reactivity is expected, even in the presence of different isoforms. Table 52 summarizes the results of experiments where three GBS 59 recombinant polypeptides from three different strains (CJB111, 515 and 2603) were used to immunize mice that were then challenged with homologous and heterologous strains. With the exception of 2603 strain, the protein is well expressed on the surface (i.e. the Δ-mean is greater than 200 channels) and confers protection against homologous challenge. In the case of mice immunized with the GBS 59^{CJB111} variant, the challenge with the heterologous strain 515 resulted in a low survival rate, confirming that the two polypeptides, although representing the same isoform, are not cross-protective in the animal model.

TABLE 52
GBS cross-reactivity.

| | | Protection | | | |
|---|---|---|---|---|---|
| | | GBS 59 | | | |
| Challenge | FACS | (Variant CJB111) | | PBS | |
| strain | (α-cjb111) | alive/ | | alive/ | |
| (serotype) | Δ-Mean | treated | % survival | treated | % survival |
| CJB111 (V) | 493 | 50/74 | 68 | 24/99 | 24 |
| 7357 B (Ib) | 596 | 30/54 | 56 | 7/40 | 17 |
| 515 (Ia) | 583 | 6/38 | 16 | 3/28 | 11 |
| | | Protection | | | |
| | | GBS 59 | | | |
| Challenge | FACS | (Variant 2603) | | PBS | |
| strain | (α-2603) | alive/ | | alive/ | |
| (serotype) | Δ-Mean | treated | % survival | treated | % survival |
| 3050 (II) | 570 | 37/40 | 92 | 5/29 | 17 |
| 18RS21 (II) | 380 | 12/30 | 40 | 1/40 | 2 |
| 2603 (V) | 186 | 15/78 | 19 | 16/75 | 23 |
| | | Protection | | | |
| | | GBS 59 | | | |
| Challenge | FACS | (Variant 515) | | PBS | |
| strain | (α-515) | alive/ | | alive/ | |
| (serotype) | Δ-Mean | treated | % survival | treated | % survival |
| 515 (Ia) | 583 | 24/43 | 56 | 6/53 | 11 |

### Example 23

### Pili are Immunogenic in Humans During Infection

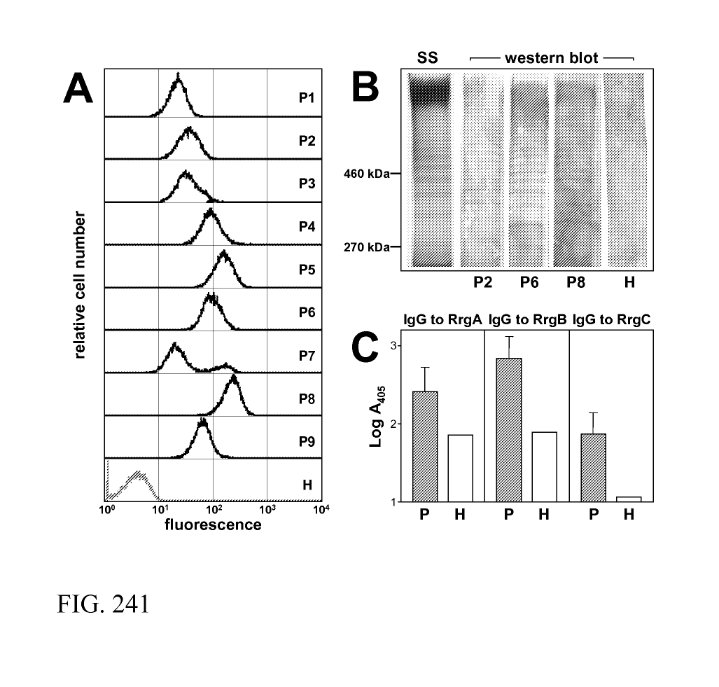

Human sera from 9 patients diagnosed with pneumococcal disease were analyzed by FACS for their ability to recognize whole cell pneumococcal preparations of the serotype 4 S. pneumoniae strain TIGR4. All 9 sera were able to recognize TIGR4 bacteria, while a serum from a healthy donor did not produce appreciable positivity (FIG. 241A). To find out whether the antibodies induced during infection recognize pili, we tested the human sera by western blot against a TIGR4 pilus-enriched mutanolysin preparation, which in SDS-PAGE forms a typical ladder of high molecular mass pilus polymers. All 9 individual patient sera recognized the pilus ladder, while the serum from a healthy donor was negative (FIG. 241B). To investigate whether, in addition to the ladder, the human sera recognized the individual pilus subunits, a pool of sera from the 9 patients was tested by western blot against recombinant pilus subunits. RrgA and RrgB were recognized, while there was no detectable recognition of RrgC.

Serum antibodies against each of the three pilus subunits were quantified by ELISA, presenting marked differences in their relative abundance. The highest specific IgG level was directed against RrgB, followed by RrgA and RrgC (FIG. 241C). These results might reflect, at least in part, the relative abundance of the three subunits in the pilus, since RrgB, which constitutes the backbone, is the most abundant pilus component, followed by RrgA and RrgC (Barocchi 2006). Differential epitope exposure of the three subunits in the assembled native pilus could also contribute to preferentially direct the immune response to one subunit rather than another, a possibility that cannot be excluded and requires further investigation for clarification. In the healthy donor serum, very low but still measurable levels of specific IgG against pilus antigens were detected by ELISA (FIG. 241C). This may be reasonably ascribed to an immune response previously developed against S. pneumoniae by this subject. A larger study with sera from patients with pneumococcal disease and from healthy people could be used to identify the negativity threshold for ELISA detection of specific IgG against pilus antigens.

### Example 24

### Native and Recombinant Pilus Subunits are Immunogenic in Mice

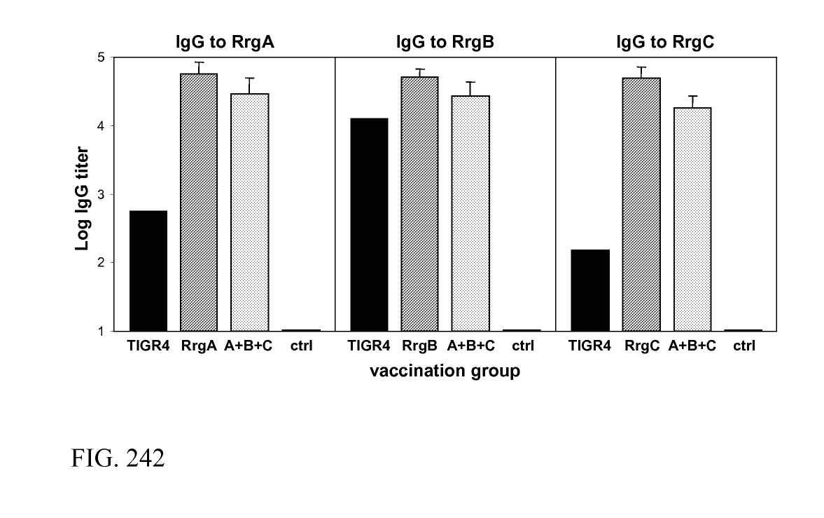

Mice vaccinated with heat-inactivated TIGR4, containing native pilus structures, generated serum antibodies against recombinant pilus antigens, as evaluated by ELISA on sera obtained after the third immunization. The highest response was detected against the main pilus subunit RrgB , followed by RrgA and RrgC (FIG. 242), similarly to that observed for the human sera.

In order to find out whether such a difference in antibody response was due to the pilus structure or to the intrinsic immunogenicity of the pilus subunits, serum IgG response was also quantified by ELISA in mice that were immunized with recombinant pilus subunits (FIG. 242). Immunization with the individual recombinant pilus antigens (20 μg each) elicited high IgG response, sera becoming titrable at 1:50,000÷1:100,000 dilution, and the antibody titers to the three pilus subunits were comparable Immunization with the combination of RrgA+B+C also elicited high IgG levels against each of the three antigens, with titers slightly reduced, consistently with the lower antigen dose used (10 μg each) (FIG. 242). Specific IgG titers were undetectable in control groups (adjuvant plus saline).

When mice were immunized with the combined pilus antigens RrgA+B+C and Al(OH)₃ as an adjuvant, high IgG response was also induced, even though slightly lower than that obtained with Freund's adjuvant. These results indicate that each of the three pilus subunits has similar immunogenicity. Thus, the differences of IgG levels against each of the pilus subunits observed both in infected humans and in TIGR4-immunized mice should be most likely ascribed to the composition and structure of the native pilus.

### Example 25

### Immunization With Recombinant Pilus Antigens is Protective in Mice

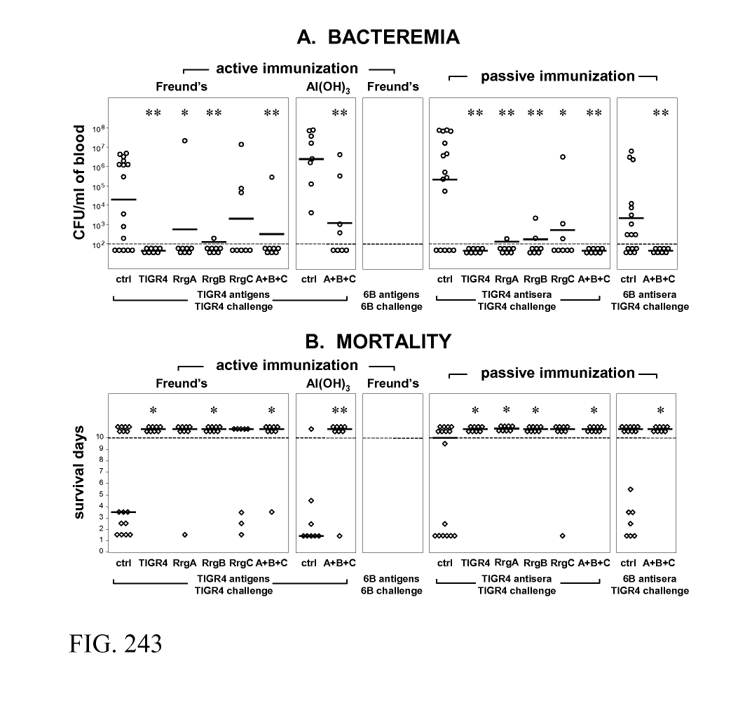

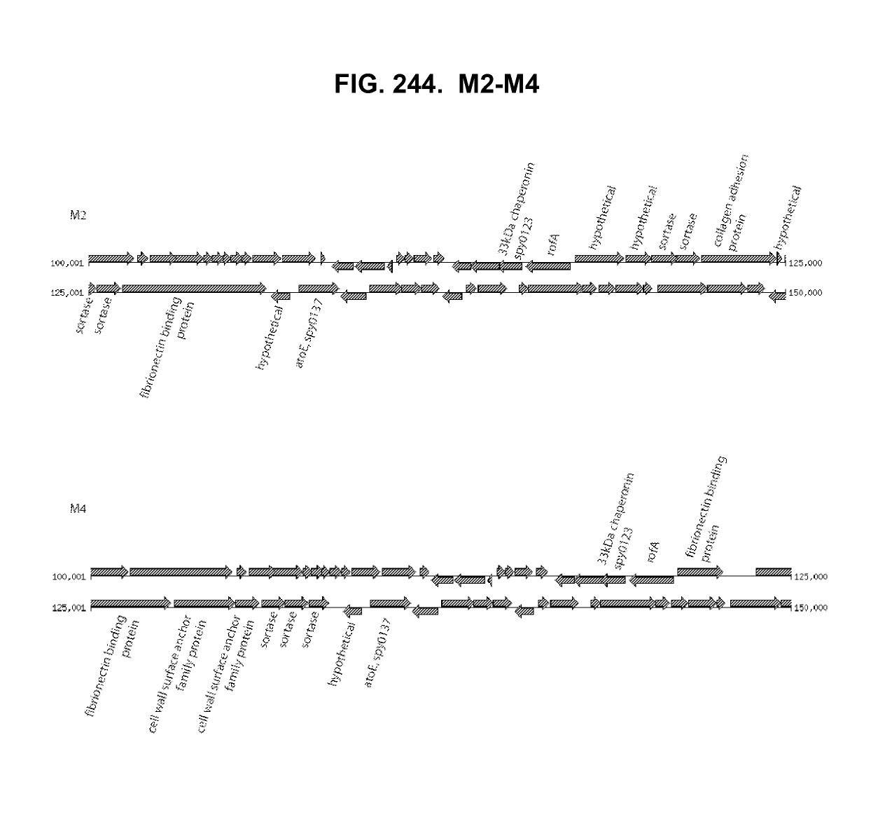

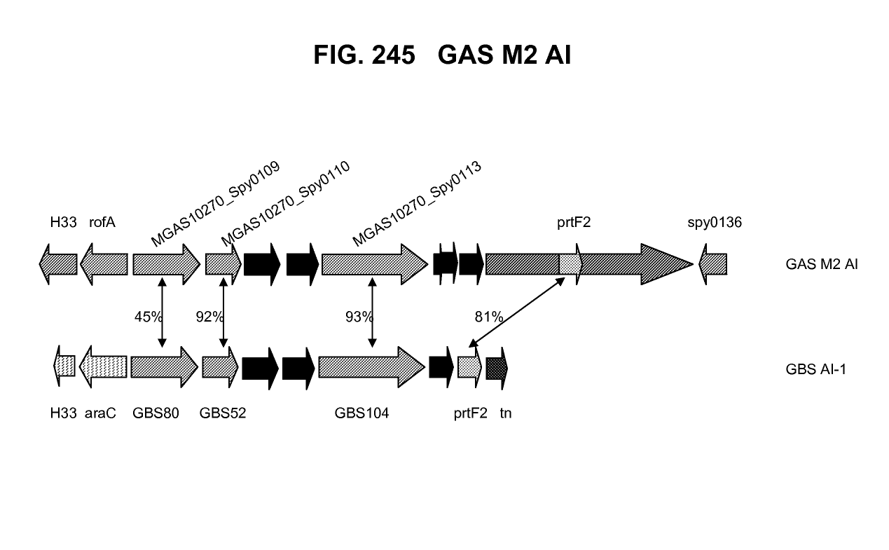

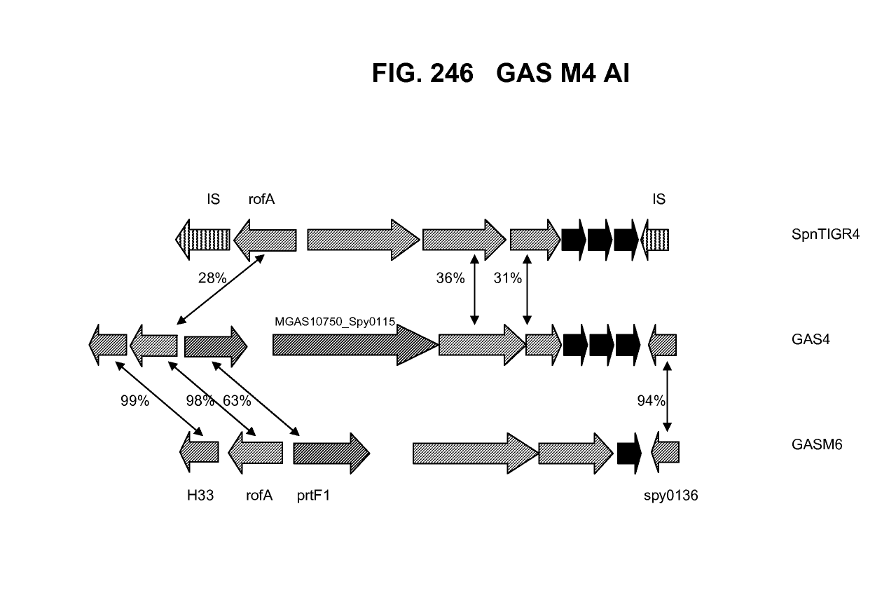

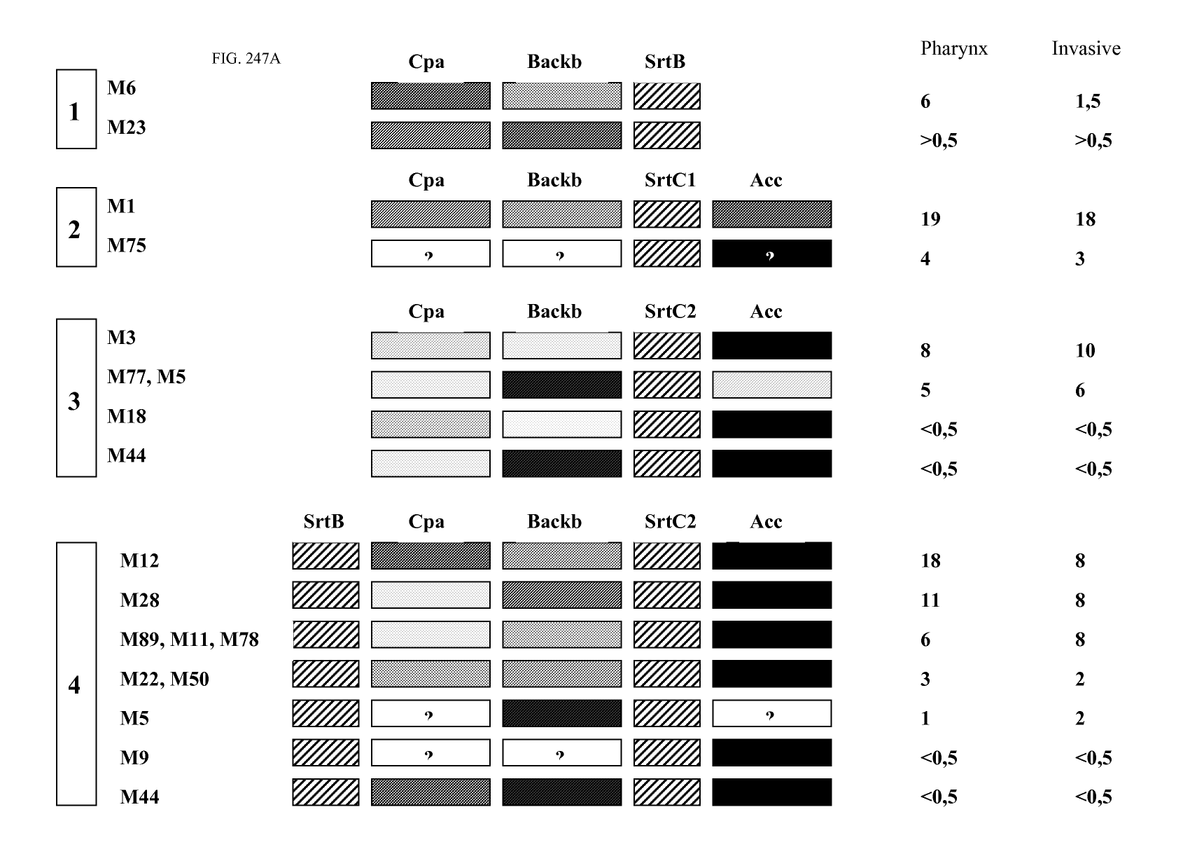

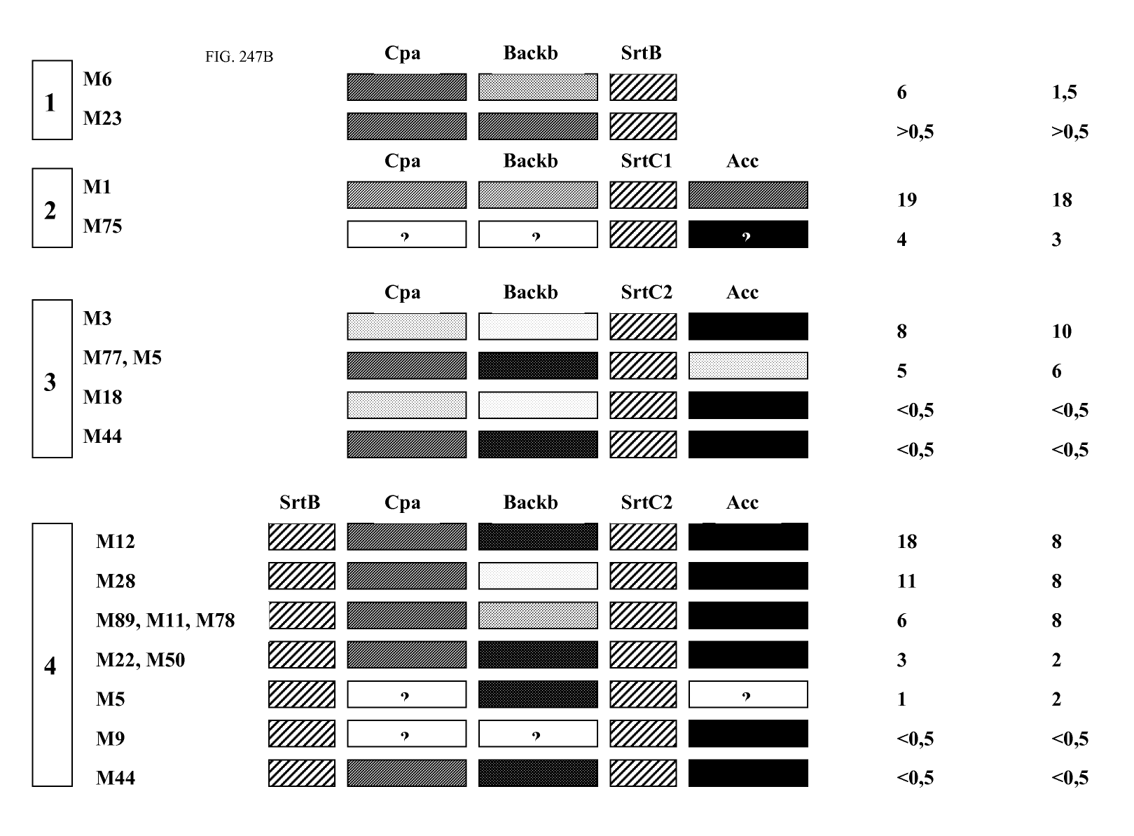

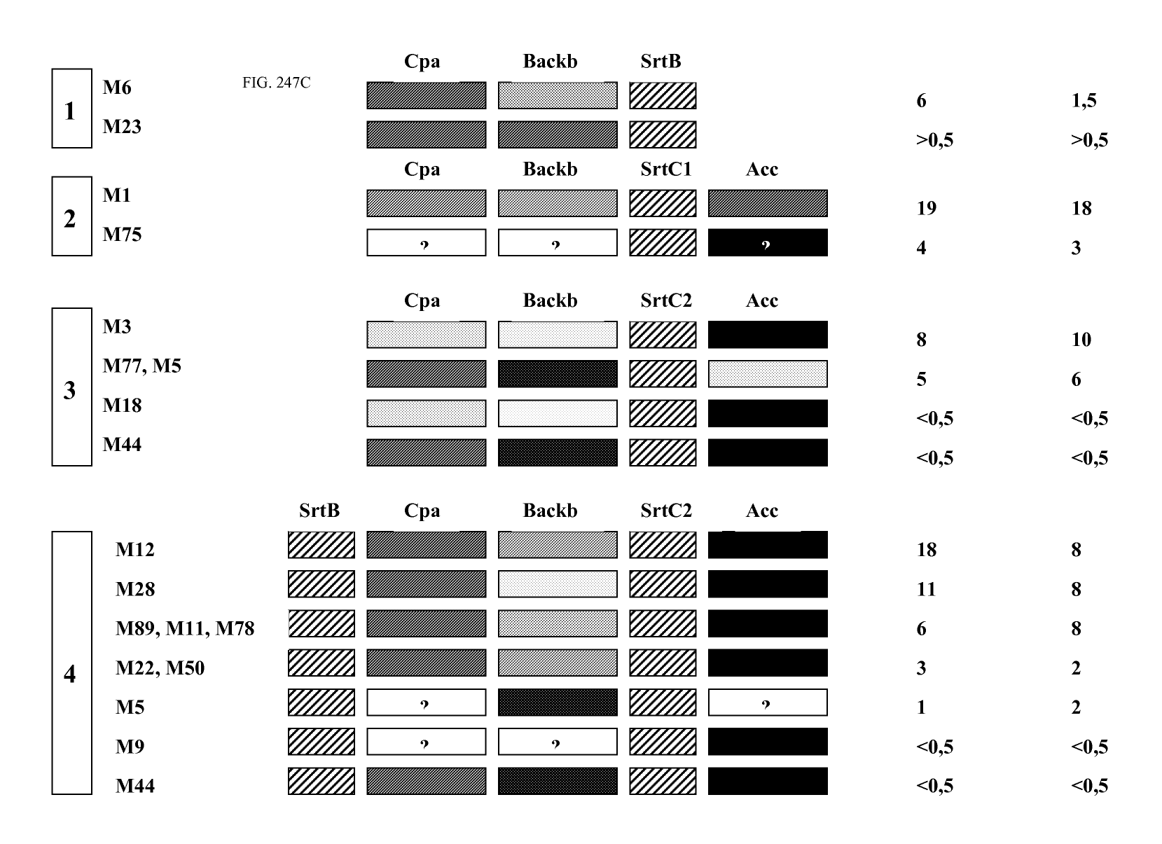

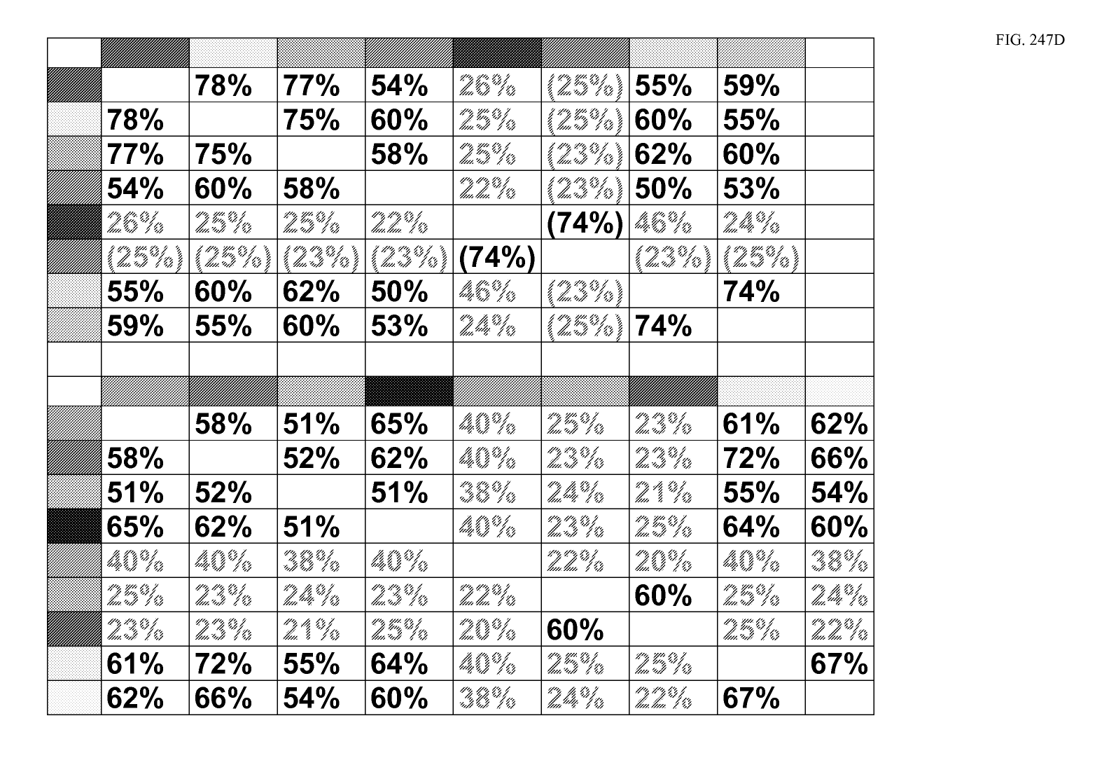

Mice were immunized intraperitoneally with recombinant pilus antigens, alone or in combination, then challenged intraperitoneally with 10² CFU of TIGR4 per mouse, a dose previously observed to cause high levels of bacteremia 24 h post-challenge and early death in naïve mice. Bacteria in the blood were quantified 24 h post-challenge. As shown in FIG. 243, in this model, control animals, receiving adjuvant plus saline, had a geometric mean of >10⁴ CFU/ml, including 7 mice with >10⁶ CFU/ml and 5 mice with no detectable bacteremia (FIG. 243A); 9 out of 16 mice did not survive at 10 days (FIG. 243B). In marked contrast, no bacteremia was detected in any of the mice vaccinated with the whole TIGR4 bacteria (FIG. 243A), and all mice of this group were alive at 10 days (FIG. 243B). All groups of mice vaccinated with recombinant pilus antigens showed lower bacteremia and increased survival, as compared with the control groups receiving adjuvant plus saline. The best efficacy was shown by RrgB, which afforded a protection similar to the whole TIGR4 bacteria, with only 1/8 mice bacteremic and 100% survival at the endpoint. The groups vaccinated with RrgA or with the combination RrgA+B+C also resulted protected, with only 1/8 mice bacteremic and 7/8 mice surviving challenge in each group. Finally, the mice vaccinated with RrgC showed only limited protection. Both in terms of bacteremia and survival, all groups immunized with pilus antigens gave results not statistically different (P>0.1) from those of the group vaccinated with heat-inactivated TIGR4, which resulted completely protected. The combination RrgA+B+C showed similar protective efficacy when Freund adjuvant was replaced by Al(OH)₃ (FIG. 243).

Interestingly, in vaccinated groups, infection and death correlated with low specific antibody titers against the three pilus subunits, suggesting the relevance of antibody response in the observed protection.

In order to further investigate whether the protective efficacy of pilus subunits is antibody-dependent, we tested mouse antisera raised against recombinant pilus antigens for their protective ability by passive transfer. Immune sera were intraperitoneally injected in mice prior to challenge with 10² CFU of S. pneumoniae TIGR4 per mouse. As shown in FIG. 243A, 24 h post-challenge, control animals presented a geometric mean of >10⁵ bacteria per ml of blood, with 10/16 mice having values >10⁵ CFU/ml, one mouse <10⁵ CFU/ml, and 5 mice with no detectable bacteremia. Ten days post-challenge, 8/16 control mice were still alive (FIG. 243B). All 8 mice receiving anti-TIGR4 serum presented undetectable bacteremia and survived at 10 days (FIG. 243B). All groups that received antisera against recombinant pilus antigens showed reduced bacteremia and increased survival time, as compared with the control group.

The passive transfer of anti-RrgA+B+C serum resulted in undetectable bacteremia at 24 h (FIG. 243A) and survival at the endpoint (FIG. 243B) for all 8 mice. Also, after passive transfer of either anti-RrgA or anti-RrgB serum, only 1 and 2 mice, respectively, were found bacteremic 24 h post-challenge (FIG. 243A), and 8/8 mice in each group survived lethal challenge (FIG. 243B).

Finally, passive transfer of anti-RrgC serum resulted in 5/8 mice with no detectable bacteremia (FIG. 243A), and 7/8 mice survived challenge (FIG. 243B). Similarly to that obtained with active immunization, all groups that received antisera raised against pilus antigens showed bacteremia and survival not statistically different (P>0.1) from those of the group that received anti-TIGR4 antiserum, which resulted completely protected. These results indicate that antibodies play a relevant role in the protective effect these antigens elicit.

The observation that, both by active and passive immunization, RrgA and RrgB are much more effective than RrgC in protecting mice against lethal challenge, even though all three antigens elicit comparable specific antibody titers, can be explained also in this case by the different relative abundance of these antigens in the native pilus. In fact, the efficacy of high antibody titers to RrgC can be hampered by the relatively low availability of their target in the infecting bacteria, that is not the case for the more abundant RrgB and RrgA.

Moreover, passive transfer of mouse immune serum raised against RrgA+B+C_{6B} was able to protect mice against heterologous challenge with 10² CFU of TIGR4. All 8 mice receiving anti-RrgA+B+C_{6B} antiserum were not bacteremic 24 h post-challenge and were still alive at 10 days. (FIG. 243) These preliminary results suggest the possible cross-protective ability of pilus subunits against different S. pneumoniae serotypes.

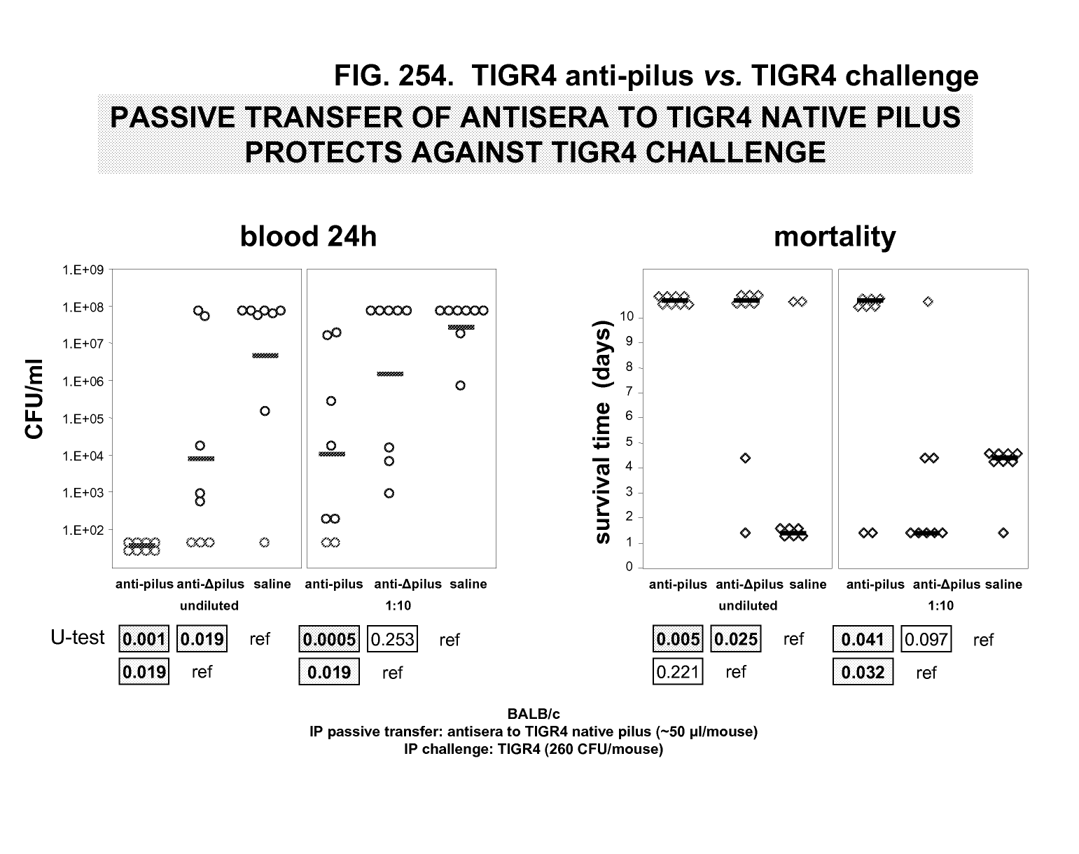

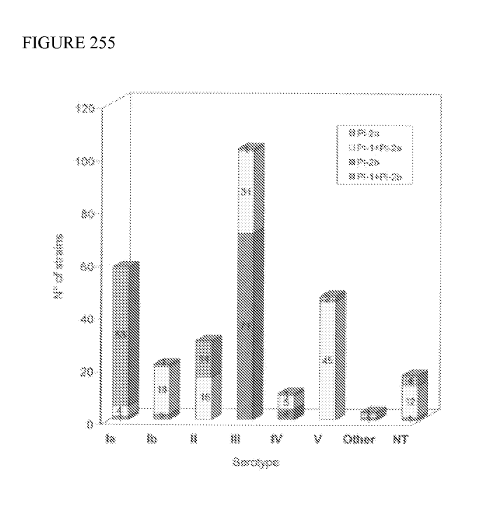

FIG. 254 demonstrates that that passive transfer of antisera to TIGR4 native pilus protects against TIGR4 challenge.

These examples provide evidence that the three S. pneumoniae pilus subunits, RrgA, RrgB and RrgC, are naturally immunogenic, and that immunization of mice with the three recombinant proteins elicits high antibody titers. Both active immunization with the three recombinant pilus components and passive transfer of antisera against these antigens is protective in mice against subsequent lethal challenge, RrgB and the combination of RrgA+B+C showing the best overall efficacy, followed by RrgA and RrgC. Although pilus structures are not universal in pneumococcal strains, the ability of the pilus recombinant proteins to protect mice against infection suggests their use as potential components of a multi-protein vaccine as an alternative capsule-independent strategy to protect against S. pneumoniae.

### Example 26

### Cloning, Expression and Purification of RrgA, RrgB and RrgC (Examples 23-25)

Standard recombinant techniques were used for nucleic acid cloning and restriction analyses. Briefly, genomic DNA from TIGR4 S. pneumoniae strain was prepared using the Wizard genomic DNA purification Kit (Promega). PCR was carried out with Expand High fidelity PCR system (Roche) according to the manufacturer's instructions. Primers were as follows:

rrgA:

(SEQ ID NO: 467)

5′-AGTTGCTGCTAGCGAAACGCCTGAAACC-3′ (forward),

(SEQ ID NO: 468)

5′-CAGTTCGCTCGAGTTCTCTCTTTGGAGG3′ (reverse);
rrgB:

(SEQ ID NO: 469)

5′-GTGCGTGCTAGCGCTGCAACAGTTTTTGCGGCTGGG-3′
(forward),

(SEQ ID NO: 470)

5′-CAGCGTCTCGAGAGTGATTTTTTTGTTGACTACTTT-3′
(reverse);
rrgC:

(SEQ ID NO: 471)

5′-GTGCGTGCTAGCCATGCAGTCCAAGCGCAAGAAGAT-3′
(forward),

(SEQ ID NO: 472)

5′-CAGCGTCTCGAGATCAATCCGTGGTCGCTTGTTATT-3′
(reverse).

The amplification products were purified, digested with the appropriate enzymes (NdeI and XhoI) and ligated in a His6 expression vector, pet21b+ (Novagen). The resulting plasmids were introduced into E. coli DH5α for sequence analysis and in E. coli strain BL21 star (DE3) for protein expression.

IPTG-induced recombinant E. coli cultures, expressing His-tagged RrgA, RrgB and RrgC proteins, were harvested and subjected to lysis by lysozyme in a BugBuster (Novagen), Benzonase Nuclease (Novagen) solution containing proteinase inhibitors. After centrifugation at 100,000 rcf for 1 h at 4° C., the soluble fraction was subjected to metal chelate affinity chromatography on His-Trap HP columns (GE Healthcare) equilibrated and eluted according to manufacturer's instructions. Purity was evaluated by scanning densitometry of Coomassie Blue-stained SDS-PAGE: fractions corresponding to >90% purity were used. Pooled fractions were dialysed overnight against 0.9% NaCl and stored at −80° C. until further use. Protein concentration was determined by scanning densitometry of Coomassie Blue-stained SDS-PAGE using a BSA standard and measuring Absorbance at 280 nm of the protein solution (NanoDrop).

Bacterial culture. Bacteria were grown at 37° C. under 5% CO₂ on Tryptic Soy Agar (Becton Dickinson) with 5% sheep blood, inoculated into Tryptic Soy Broth (Becton Dickinson), and further cultured until reaching OD₆₀₀=0.2 (=10⁷ CFU/ml).

Protein expression and purification. Genomic DNA was prepared from TIGR4 or 6B strains using the Wizard Genomic DNA Purification Kit (Promega). PCR was done with Expand High Fidelity PCR System (Roche). Primers are listed in Table 1. PCR products were digested with NdeI and XhoI (New England Biolabs), ligated in pET21b+ (Novagen), and the plasmids introduced into E. coli BL21 Star (DE3). Soluble recombinant pilus subunits corresponding to the sequence of TIGR4 (RrgA, RrgB, RrgC) or 6B (RrgA_{6B}, RrgB_{6B}, RrgC_{6B}) were purified by His-Trap HP (GE Healthcare). Protein purity and concentration were determined by SDS-PAGE scanning densitometry.

Mice and study design. Animal experiments were done in compliance with the current law. Six-week-old specific-pathogen-free female BALB/c mice (Charles River) were immunized intraperitoneally (i.p) on day 0, 14 and 28 with RrgA, RrgB, RrgC (20 μg), a combination RrgA+B+C or RrgA+B+C_{6B} (10 μg each), or heat-inactivated bacteria (10⁸ CFU), along with Freund's adjuvant. The combination RrgA+B+C was also given i.p. on day 0, 10 and 20, with 200 μg Al(OH)₃. Controls received an identical course of saline plus the adjuvant. Two weeks after the last immunization, each mouse was i.p. challenged with 10² CFU of TIGR4 (LD₁₀₀ in naïve mice). For passive immunization, 10-week-old mice received i.p. 50 □l of pooled mouse immune sera 15 min before lethal challenge with TIGR4 as above or with 10⁶ CFU of 6B. Bacteremia was quantified at 24 (TIGR4) or 5 h (6B), and the survival monitored for 10 days (TIGR4) or 15 days (6B) post-challenge.

FACS Analysis. TIGR4 bacteria were incubated on ice for 30 min with human sera diluted 1:50. Antibody binding was revealed by FITC-labeled anti-human IgG (Jackson ImmunoResearch) and samples analyzed by FACSCAN (Becton Dickinson).

Western blot. TIGR4 mutanolysin preparation was run on 3-8% NuPage Novex Bis-Tris Gel (Invitrogen) and blotted onto 0.45 μm nitrocellulose. Human sera were added at 1:3,000 dilution followed by alkaline-phosphatase conjugated anti-human IgG (Promega). Immunoreactive bands were visualized by the Western Blue Stabilized Substrate (Promega).

ELISA. Serial dilutions of human or mouse sera were dispensed in Maxisorp 96-well plates (Nalge Nunc Int.) coated with recombinant RrgA, RrgB or RrgC 0.2 μg/well. Antibody binding was revealed by alkaline phosphatase-conjugated anti-human (Sigma) or anti-mouse (Southern Biotechnology Ass.) IgG, followed by p-nitrophenyl-phosphate (Sigma). Absorbance was measured at 405 nm. Mouse sera were titrated using a reference line calculation program, by comparison with the reference curves. Reference consisted of pooled anti-RrgA, -RrgB or -RrgC mouse sera, which tested by ELISA at 1:100,000 dilution gave similar A₄₀₅ values, and to which the titer of 50,000 was assigned.

Statistics. Data were evaluated by one-tailed Mann-Whitney U test. P values <0.05 were considered and referred to as significant.

### Example 27

### Pilus Like Structures Promote Cell Auto Aggregation and Biofilm Formation in Group A Streptococcus pyogenes (GAS)

Bacterial Strains, Media, and Growth Conditions. GAS M1 strain SF370 was provided by University of Siena, Italy. Wild-type and mutant strains were grown at 37° C. or 30° C. in Todd-Hewitt medium supplemented with 0.5% yeast extract (THY) (Difco), or THY agars supplemented with 5% defibrinated sheep blood. L. lactis subspecies cremoris MG1363 was grown at 30° C. in M17 (Difco) supplemented with 0.5% glucose (GM17). 20 μg/ml chloramphenicol was used in selective medium.

Construction of GAS deletion mutants and complementation. In-frame deletion and complementation mutants of GAS strain SF370 were constructed as described before (Mora et al., 2005). Briefly, mutations were constructed by using splicing-by-overlap-extension PCR (Horton, et al., 1990). The PCR deletion construct was cloned in the temperature-sensitive allelic exchange vector pJRS233, and transformation and allelic exchanges were performed as described in (Frameson et al., 1997; Caparon and Scott 1991 and Perez-Casal et al., 1993). Transformants were selected on THY plates with 1 μg/ml erythromycin (Sigma) at 30° C. Drug-sensitive colonies were screened and deletions were confirmed by PCR assay. The complementation vectors pAM401::128 and pAM401::129 were constructed with the appropriate primers to amplify the fragment that includes the spy0128 or spy0129 gene, the predicted promoter and the P-independent terminator.

L. lactis transformation with GAS pilus region. The complementation vector pAM401::pilM1 was constructed with the appropriate primers to amplify the fragment that includes the genomic region comprised between spy0126 to spy0130. The fragment was cloned in the pAM vector containing the promoter and terminator regions of GBS adhesin island-2 (Buccato et al., 2006). The vector was then inserted in L. lactis MG1363 competent cells by electroporation, and the transformants were selected on GM17 plates with 20 μg/ml chloramphenicol. Drug-resistant colonies were screened by PCR. The expression of pilus subunits and their assembly into a covalently bound polymeric structure was confirmed by western blot analysis, using polyclonal sera obtained from mice immunized with the corresponding GAS pilus proteins.

Immunoblots on bacterial cell-wall fractions. Bacterial cell-wall fractions were prepared as described previously. In particular, bacteria grown in THY to OD₆₀₀=0.4 at 37° C. were pelleted, washed once in PBS, suspended in 1 ml of ice-cold protoplasting buffer [40% sucrose; 0.1 M KPO4, pH 6.2; 10 mM MgCl2; Complete EDTA-free protease inhibitors (Roche); 2 mg/ml lysozime; 400 units of mutanolysin (Sigma)] and incubated at 37° C. for 3 h. After centrifuging at 13,000×g for 15 min, the supernatants (cell-wall fractions) were frozen at −20° C.

Cell-wall preparations were then separated by 3-8% gradient gels (NuPAGE Tris-acetate gels, Invitrogen) and transferred to nitrocellulose membranes (Bio-Rad) for immunoblot analysis with mouse polyclonal antisera at a 1:500 dilution obtained as described before (Mora et al., PNAS2005) and ECL enhanced chemiluminescence detection (SuperSignal West Pico chemiluminescent substrate, Pierce). The secondary antibody (ECL, horseradish-peroxidase-linked anti-mouse IgG, GE Healthcare) was used at a 1:5,000 dilution.

Electron Microscopy. GAS was grown on THY blood agar plates and resuspended in PBS. Formvar-carbon-coated nickel grids were floated on drops of bacterial suspensions for 5 min, fixed in 2% PFA for 5 min, and placed in blocking solution (PBS containing 1% normal rabbit serum and 1% BSA) for 30 min. The grids were then floated on drops of primary antiserum diluted 1:20 in blocking solution for 30 min at RT, washed, and floated on secondary antibody conjugated to 10 nm gold particles diluted 1:10 in 1% BSA for 30 min. Bacteria were then fixed again for 10 min. The grids were washed with PBS then distilled water and air dried and examined using a TEM GEOL 1200EX II transmission electron microscope. Preimmune serum from the same animals were used as a negative control.

Light microscopy. L. lactis was grown in GM17 to mid-log phase. 20 μl of bacterial suspension was placed on a glass slide, covered with a coverslip and observed with a Bio-Rad confocal scanning microscope.

Confocal microscopy. GAS aggregation was observed by confocal laser scanning microscopy (CLSM). In particular, approximately 2×10⁸ bacteria grown to OD₆₀₀=0.2 were seeded in 12-well plates containing sterile glass cover-slips coated with poly-lysine and were left growing upon the cover-slips up to the late exponential phase, when aggregation reaches a maximum. Samples were then fixed with paraphormaldeyde 2.5% for 15 minutes, washed with PBS and blocked for 15 minutes. Then samples were incubated with primary antibodies (rabbit-anti-GAS and mouse-anti-spy0128) for 1 h at RT, washed in blocking solution and incubated for 30 minutes at RT with secondary antibodies: Alexa Fluor dye 647 goat anti-rabbit and Alexa Fluor dye 568 goat anti-mouse (Molecular Probes). Cover-slips were then washed with blocking solution and mounted on glass slides with the Slow Fade reagent kit (Molecular Probes) containing 4_{—},6_-diamidino-2-phenylindole dihydrochloride before they were viewed on a Bio-Rad confocal scanning microscope.

For aggregation on eukaryotic cells surface, 2×10⁵ Detroit-562 cells were seeded on glass cover-slips coated with polylysine in 12-well plates. The day after 5×10⁸ bacterial cfu of each strain from a logarithmic growth were extensively pipetted to break possible aggregates and used to infect mono-layers at 37° C. in a 5% CO2 atmosphere. After 15 minutes cells were washed 3 times with PBS to remove the unattached bacteria, and infection was let continue to 30, 60 and 120 minutes. Samples were then washed again, fixed, blocked and stained with rabbit-anti-GAS as a primary antibody and Alexa Fluor dye 488 goat anti-rabbit (Molecular Probes) as a secondary antibody. Cells were stained with phalloidin conjugated with Alexa Fluor dye 647 (Molecular Probes). Mounting and viewing were performed as already described.

For each strain of bio-film used in CLSM studies, a 1:10 dilution of an overnight culture in C-medium (Lyon et al., 1998) at 37° C. was inoculated at RT on poly-lysine coated glass sterile cover-slips positioned in 50 ml falcon containing 10 ml of fresh C medium, as described elsewhere (Cho and Caparon, 2005). Five ml of C medium were replaced every 24 hours and preparations were collected after desired time points of growth (24, 48 and 72 hours). Samples were then fixed, blocked and stained with rabbit-anti-GAS and mouse-anti spy0128 as primary antibodies and Alexa Fluor dye 647 goat anti-rabbit and Alexa Fluor dye 568 goat ant-mouse as secondary antibodies (Molecular Probes). Exopolysaccharides (EPS) were stained by the FITC-conjugated lectin Concanavalin A (Sigma). Mounting and viewing were performed as already described. Three-dimensional immunofluorescence images were reconstructed from 0.5-μm confocal optical sections by using VOLOCITY 3.5 (Improvision, Lexington, Mass.).

Bio-film formation assay. For each strain, a 1:10 dilution of an overnight culture in C-medium ( ) at 37° C. was inoculated in 1 ml of fresh medium in 24-well plates in triplicate. Plates were incubated at room temperature for 16-24-48-72 h, changing medium every 24 h. At each time point, the medium was removed and adherent bacteria were stained with crystal violet (0.2% in distilled water) by incubating at room temperature for 10 minutes. Crystal violet was then eluted with 1% SDS and bio-film formation was quantified by measuring the optical density at 540 nm.

Eukaryotic cell cultures. The human pharynx carcinoma cell line Detroit-562 (ATCC CCL-138) was cultured in Dulbecco's modified Eagle's medium (EMEM; Life Technologies Gibco BRL) supplemented with 10% FCS (Life Technologies) and 5 mM glutamine (Life Technologies) at 37° C. in an atmosphere containing 5% CO₂. For adherence assays, cells were resuspended at a concentration of approximately 3×10⁵ cells/ml in EMEM, and seeded into 24-well tissue culture plates (Nunc), which were then incubated for 24 h. For microscopic assays, approximately 6×10⁵ cells/ml were seeded onto 12-mm-diameter glass coverslips placed on the bottom of 24-well tissue culture plates.

Adherence assay. Bacteria from exponential phase cultures were collected by centrifugation (3000×g, 5 min), resuspended in conditioned EMEM and used to infect Detroit 562 cells monolayers for 5, 15, 30 and 120 min at 37° C. in a 5% CO₂ atmosphere. A Multiplicity of Infection (MOI) of 100:1 (for GAS strains) or 10:1 (for L. lactis strains) were used. After infection, the wells were extensively washed with PBS to remove unattached bacteria, incubated with 1% saponin to lyse eukaryotic cells, and adherent bacteria were plated for enumeration. Adherence results were expressed as the average number of bacteria recovered per ml for three independent determinations in a single assay and the percentage of adherence was calculated using the following equation: bacteria recovered after infection (cfu/ml)/bacteria inoculated (cfu/ml)×100. Tests were repeated at least three times and results are expressed as the averages+SD of three experiments performed in triplicate.

Statistics. T student test was used to compare biofilm formation and cell adhesion of wild type and mutant strains. Data with p value <0.05 were reported as statistically significative.

### Pilus Dependent Bacterial Aggregation During In Vitro Growth

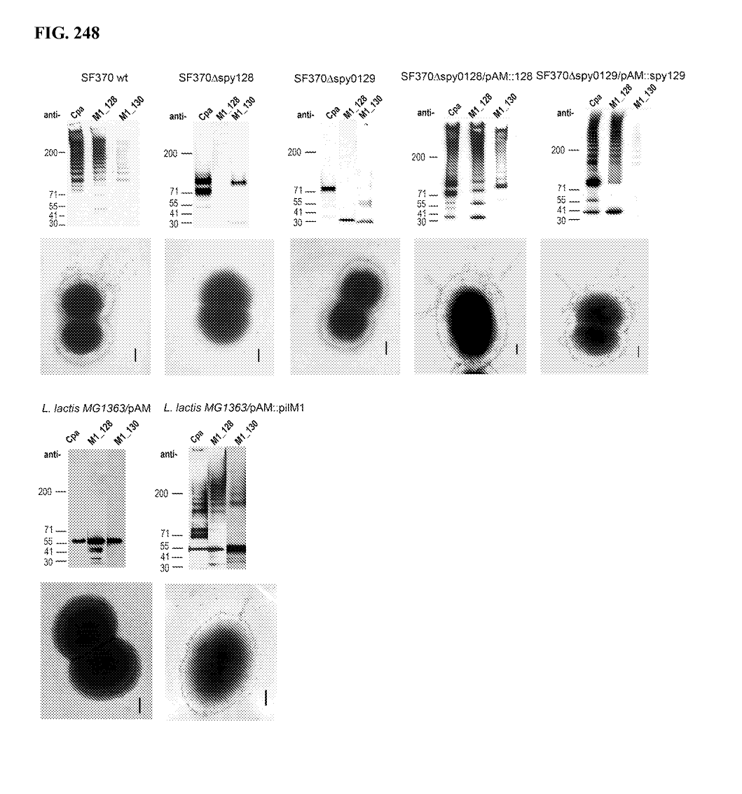

We previously showed that S. pyogenes can display pilus-like structures on their surface and that pili and their assembly machinery are encoded in a 11 kb highly variable pathogenicity island known as the fibronectin binding, collagen binding, T-antigen (FCT) region (Mora et al., 2005). In the transformable strain M1_SF370, the genes for the three pilin components and the sortase enzyme involved in pilus assembly are located in the FCT-2 variant region. In frame deletion of either the pilus backbone encoding gene (M1_{—}128) or the C1 sortase (M1_{—}129) resulted in abolished polymerization of all three pilin proteins, whereas the respective complemented strains produced again pili (FIG. 248).

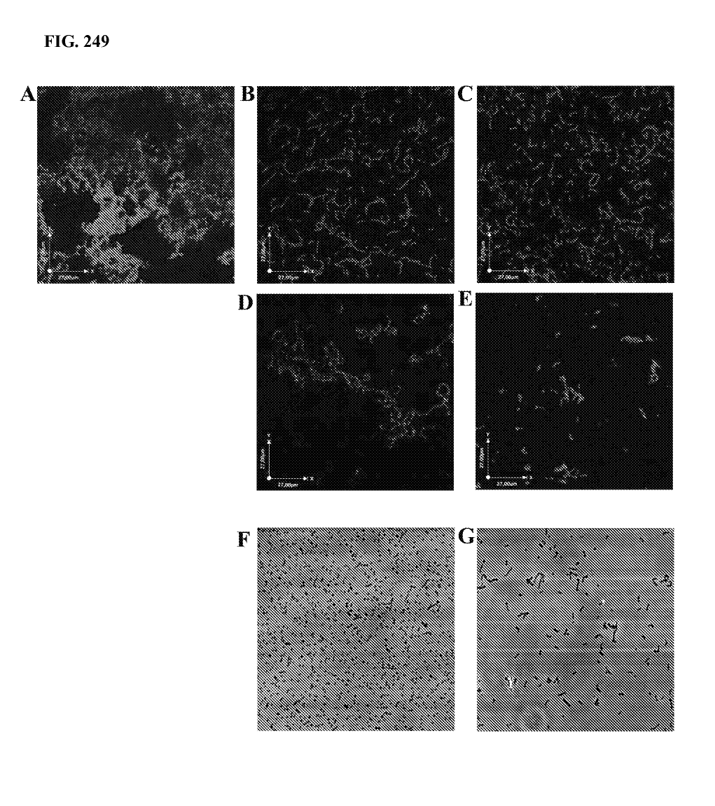

As a first step to investigate the phenotype of the two GAS derivatives unable to form pili, in vitro growth of the two mutants was compared to wild-type. When SF370 was grown in liquid medium, it started forming large visible aggregates from the early exponential growth phase, which progressively precipitated to the bottom of the tube. Although their growth rate was unaffected, the two mutant strains remained in solution for a longer period. This observation led us to further investigate whether pili could be involved in self-aggregation of bacteria. Using Confocal Laser Scanning Microscope (CLSM) we observed the vast aggregates formed by wild type SF370 grown to exponential phase and double labeled with sera raised against whole GAS bacteria and with Spy128 purified recombinant protein (FIG. 249A). Conversely, ΔSPy128 and ΔSpy129 mutants were not stained with anti-Spy128 and they formed only the typical streptococcal chains without any or very low aggregation (FIGS. 249B and C). Plasmid mediated complementation resulted in a partially restored capacity to self aggregate, even if not all the bacteria were labeled with anti-Spy 128, perhaps due to plasmid instability (FIGS. 249D and E).

To further test whether pili could per se be responsible of the self-aggregating phenotype, we introduced the five genes involved in GAS SF370 pilus formation into L. lactis, a non pathogenic Gram-positive microorganism which does not form aggregates during growth. Lactococcal bacteria, already shown to correctly assemble pili from Streptococcus agalactiae (Buccato et al., 2006), expressed and assembled the GAS pilin proteins in a covalently bound polymerized structure, as could be inferred from the high molecular weight pattern visible in immunoblots (FIG. 248). As hypothesized, light microscopy analysis (FIG. 249F-G) revealed that L. lactis bearing the M1 pilus region acquired the capacity to form aggregates, whereas the strain transformed with the vector alone failed to show any aggregation phenotype, strongly suggesting an involvement of the pilus region in inter bacterial attachment.

### GAS Association to Human Pharyngeal Epithelial Cells is Dependent on the Presence of Pili

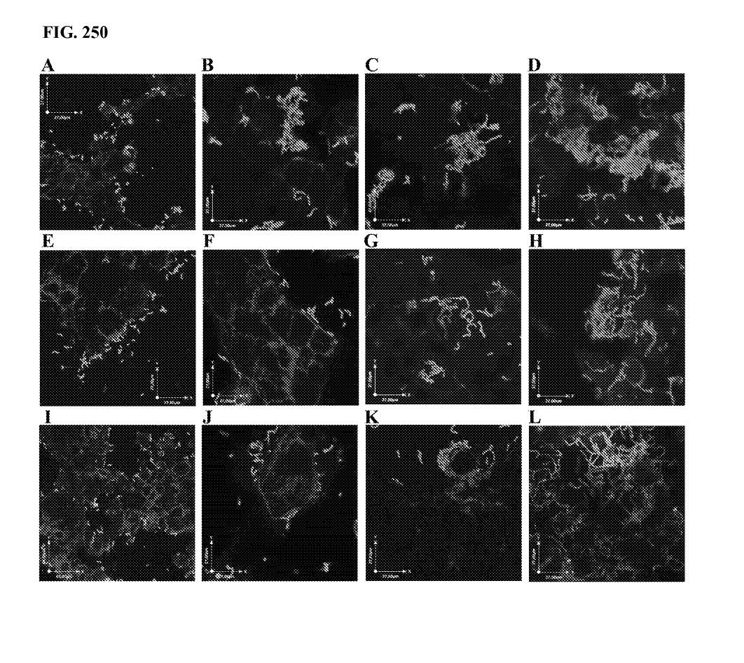

To evaluate whether the in vitro observed aggregation phenomenon could be similar to the behavior of bacteria during adhesion to host cell epithelia, we co-cultured SF370 wild type, ΔSPy128 and ΔSPy129 strains with the human pharynx cell line Detroit-562 and observed bacteria adhering to cells by confocal microscopy. In particular, 5×10⁸ bacterial cfu from a logarithmic growth were extensively pipetted to break possible aggregates and used to infect mono-layers of approximately 2×10⁵ Detroit-562 cells. After 15 minutes cells were thoroughly washed to eliminate loose-adherent bacteria and infection continued up to 30, 60 and 120 min. As shown in FIG. 250 A-D, after 30 minutes the wild type strain started forming aggregates which specifically adhered to the cell surface and became larger during longer incubation times. Conversely the two mutant strains, although adhering to cells, started forming aggregates only after 1-2 hours of infection. This suggests an involvement of GAS pili in concentrating large numbers of bacteria to the epithelial layers during the initial stages of colonization (FIGS. 250E-H and I-N).

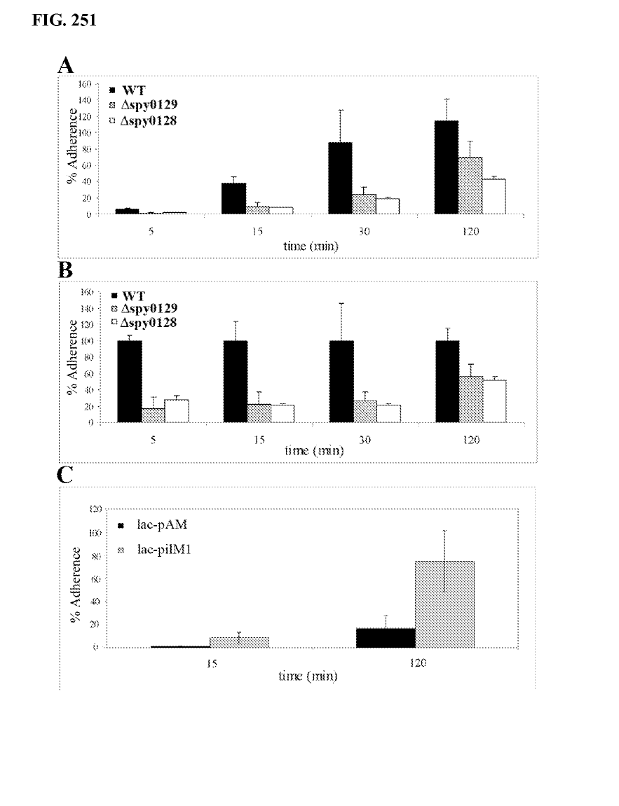

The results were confirmed by performing a classical adhesion assay in which a confluent cell monolayer was infected with 10⁸ thoroughly pipeted bacteria or its isogenic mutants ΔSPy128 and ΔSpy129 and the number of adhering bacteria after 5, 15, 30 and 120 minutes of infection was measured after extensive washing. Bacterial growth was checked by counting the total number of cfu in parallel wells and was found to be equivalent in all strains. As shown in FIG. 251A-B, the number of bacteria associated to cells was significantly lower for the two mutant strains compared to wild type after 30 and 120 minutes but also at shorter times, indicating a delay in initial adhesion as well as a slower formation of cell-associated aggregates as already described in confocal microscopy experiments. These data suggest that, like Type IV pili in V. cholerae, GAS pili contribute to cell adherence both by mediating micro-colony formation and by acting as a ligand for early binding to a surface exposed epithelial cell receptor.

The role of pili in adherence to epithelial cells was confirmed in a new adhesion assay in which 10⁷ cfu of L. lactis harboring either the GAS M1 pilus island or the recipient strain transformed with the plasmid vector as control, were co-cultured with Detroit-562 cells and adherent bacteria were counted after 15 and 120 minutes. As shown in FIG. 251C, the acquisition of the pilus island strongly increased L. lactis adhesion to epithelial cells both after 15 and 120 minutes, thus further confirming the involvement of GAS M1 pili in cell adhesion. All these data strongly suggest that pili facilitate the early stages of GAS cell adhesion to pharyngeal eukaryotic cells, probably by means of the adhesin proteins of which they are composed.

### GAS SF370 Pili are Involved in the Cell Surface Interaction Required for Bio-Film Formation

Many bacteria, including S. pyogenes aggregate during growth and form micro colonies which further develop into bio-film structures (reviewed in Hall-Stoodley et al., 2004). To investigate whether the described self-aggregation mediated by pili was instrumental to bio-film development, we performed a classical bio-film plate assay. Bacteria were incubated at room temperature in C medium in 24-well plates, and stained with crystal violet. A preliminary study indicated that GAS SF370 fully attached to polystyrene surfaces in 16 to 24 hours, whereas adhesion diminished after 48 and 72 hours. Based on these data, the capacity of wild type and its mutants to form bio-film was compared after 24 hour incubation.

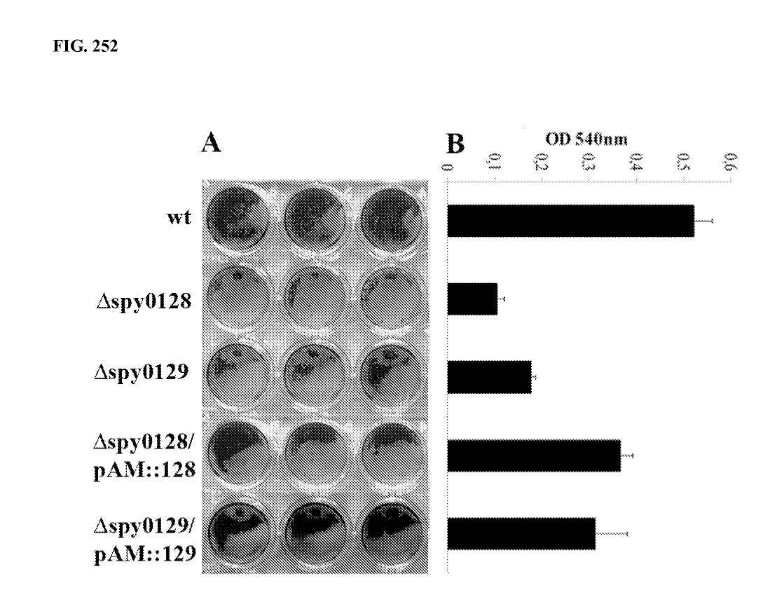

As shown in FIG. 252, wild type bacteria formed significantly more bio-film than mutants unable to form pili (p val <0.025). Moreover, when Spy128 and Spy129 genes were again introduced in the mutant strains, we observed a partially restored bio-film forming ability (p val <0.05). The number of cfu after 24 hour growth was equivalent in all strains. These data clearly demonstrate that a non polar deletion of pili considerably impairs bio-film formation.

### Pili Affect Biofilm Maturation

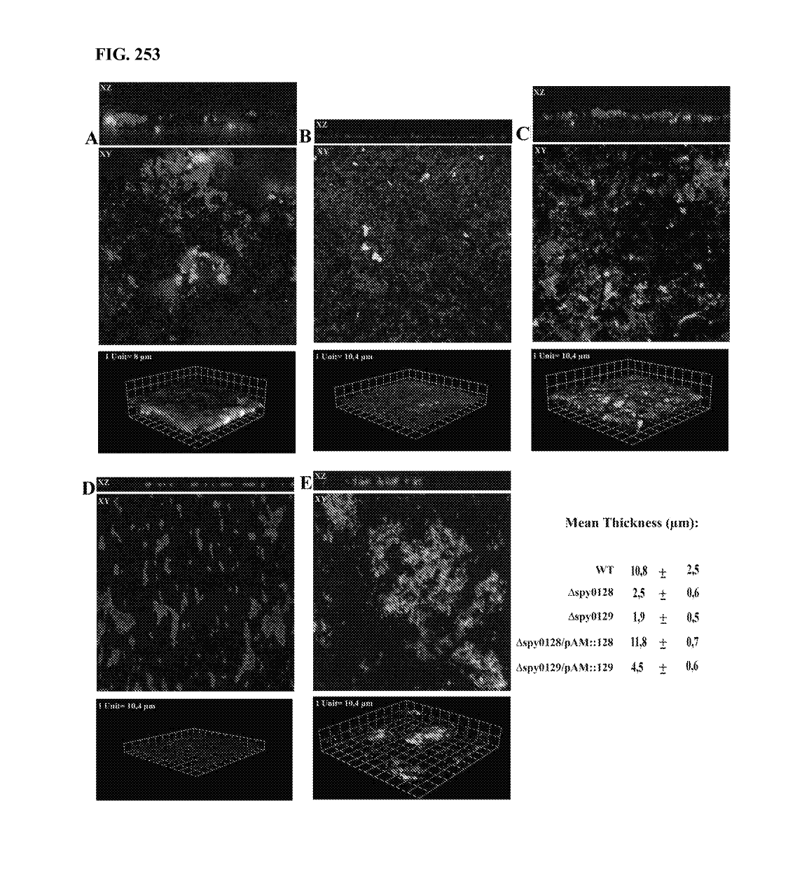

Bio-film assay on plates detects primarily the initial cell-surface interactions required for bio-film formation (O'Toole et al., 2000). To analyze subsequent stages of bio-film maturation wt, ΔSPy128, ΔSPy129 and their complemented strains ΔSPy128(pAM128) and ΔSPy129(pAM129) were grown on poly-lysine coated glass cover-slips, double labeled with anti GAS and anti Spy128 sera and examined by confocal microscopy. After 72 hours the bio-film formed by the wild-type strain showed an average thickness of 10.8 μm while the two mutants attached to the glass surface but failed to form a significant multilayered structure and thus a mature bio-film (three dimensional and multilayered) (FIG. 253). On the other hand the complemented strains ΔSPy128(pAM128) and ΔSPy129(pAM129) produced 11.8 μm and 4.5 μm thick bio-films respectively. Furthermore, wt and complemented bacteria were able to produce the extra cellular saccharides that most bacterial cells secrete during bio-film development (EPS), stained in green by the lectin Concanavalin A labeled with FITC, whereas very low amounts of EPS could be detected in the mutant strains (FIG. 253).

### Example 27

### Pili Genomic Islands are Ubiquitous in Clinical Isolates of Streptococcus agalactiae: a Basis for a Broadly Protective Vaccine

In this example, we provide a thorough analysis of the distribution of the three pilus-like genomic islands among 289 clinical isolates of GBS collected at distant geographic sites. Moreover, sequence variability of the PI genes coding for the three structural proteins of each pilus has been determined for 186 isolates. This example has led to the definition of a combination of three antigens, one for each pilus island, that could form the basis for a broadly protective vaccine.

Bacterial strains and growth conditions. Streptococcus agalactiae (GBS) isolates used in this work were collected from patients with invasive GBS infections and asymptomatic colonization. The isolates came from three collections: the Center for Disease Control and Prevention (CDC), Atlanta, Ga. (2000 to 2003); Baylor College of Medicine (BCM), Houston (2002 to 2005) and Istituto Superiore di Sanità, Italy (1992 to 2006). Serotyping of isolates at CDC and BCM used the capillary precipitin method of Lancefield. GBS strains 2603 V/R (capsular serotype V), 515 (Ia), CJB111(V), H36B(Ib), COH1(III), used as source of DNA for amplification of pili genes, were a gift from Dr. Dennis Kasper (Harvard Medical School, Boston, USA). Bacteria were grown at 37° C. in Todd Hewitt Broth (THB; Difco Laboratories) or in trypticase soy agar supplemented with 5% sheep blood.

DNA isolation. Genomic DNA was prepared by a standard protocol for gram-positive bacteria using a NucleoSpin Tissue kit (Macherey-Nagel) according to the manufacturer's instructions. In brief, GBS isolates were grown in 10 ml of THB medium to OD600 nm 0.5. The culture was centrifuged for 10 min. at 3000 rpm, the cell pellet was resuspended in 180 μl of lysis buffer containing 20 mM Tris pH 8.0, 2 mM EDTA, 1% Triton X-100, 1 mg lysozyme (Sigma), 50 units of mutanolysin (Sigma) and incubated for 1 h at 37° C. Then 25 μl of Proteinase K (20 mg/ml) was added and samples were incubated at 56° C. for at least 1 h. When a complete lysis was obtained, 10 μl of RNase A (20 mg/ml) were added and samples were incubated for an additional 10 min at 56° C. The DNA from the bacterial clear lysates was isolated using NucleoSpin Tissue columns and eluted in sterile water.

PCR amplification and DNA sequencing. Genes were amplified using primers external to the coding sequence. The primers are listed in Table VII. Each PCR reaction was performed in 100 μl containing 100 ng of GBS chromosomal DNA, 50 μM of each primer, 200 □M of each dNTP and 0.5 U of Pwo DNA polymerase (Roche) in 1× buffer with 1.5 mM MgCl2. The reaction conditions for denaturation were 94° C. for 5 min., followed by 30 cycles (denaturation at 94° C. for 30 sec, primer annealing at 55° C. for 45 sec and extension at 72° C. for 1-2 min.). The nucleotide sequences of PCR products were determined using a BigDye Terminator V3.1 Cycle Sequencing kit (Applied Biosystem) in an ABI PRISM 3700 DNA Analyzer (Applied Biosystem).

Sequence Alignments and Phylogenetic Analysis. The percentage of sequence identity was calculated by pair wise BLAST with the VECTOR NTI SUITE 9 for PC (Informax, Bethesda), with gaps included. Protein alignments were performed by using the program CLUSTAL W (1.83) included in the GCG Wisconsin Package version 11.1. Phylogenetic trees were inferred from the protein alignments by the neighbour-joining-distance-based method and bootstrapped 1,000 times. The complete genome sequences of Streptococcus agalactiae strain 2603V/R (V), A909 (Ia) and NEM316 (III) are available under accession numbers AE009948, CP000114, AL732656. The genome sequences in assembly of strains 18RS21 (II), 515 (Ia), CJB111 (V), H36B (Ib) and COH1 (III) are available under accession numbers AAJO00000000, AAJP00000000, AAJQ00000000, AAJS00000000, AAJR00000000.

Cloning, expression and purification of recombinant proteins. Recombinant proteins were expressed in E. coli BL21DE3 cells (Novagen) as 6His-tagged fusion proteins by cloning the corresponding genes in pET24b+ (Novagen) and purified by affinity chromatography as previously reported (22). GBS strain 2603 V/R (serotype V) was used as source of DNA for cloning the sequences coding for the PI-1 proteins (TIGR annotation SAG0645, SAG0646, SAG0649) and the PI-2a LPXTG proteins (TIGR annotation SAG1408, SAG1407, SAG1404). GBS strain 515 (Ia) and GBS strain CJB111 (V) were used for cloning the sequences coding for the corresponding PI-2a backbone protein (TIGR annotation SAL1486, SAM1372) and GBS strain H36B (Ib) for the amplification of the gene coding for the PI-2a ancillary protein 1 (TIGR annotation SAI1512). GBS strain COH1 (III) was used for cloning the genes coding for the PI-2b proteins (TIGR annotation AP1-2b, BP-2b and AP2-2b). Primers were designed to amplify the coding regions without the signal peptide sequence and the 3′ terminal sequence starting from the region coding for the LPXTG motif.

Mouse immunization. Purified recombinant GBS proteins were used for intraperitoneal immunization of groups of 6- to 8-week-old CD-1 outbred mice (Charles River Laboratories, Calco, Italy). 20 μg of each protein was administered to mice on days 1 (emulsified in Complete Freund's adjuvant, CFA), 21 and 35 (in Incomplete Freund's adjuvant, IFA). Sera from each group of mice were collected on days 0 and 49, and the protein-specific immune response (total Ig) in pooled sera was monitored by ELISA.

Flow cytometry. Exponential phase grown GBS strains were resuspended in PBS containing 0.05% paraformaldehyde, and incubated for 1 h at 37° C. and then overnight at 4° C. Fixed bacteria were then washed once with PBS, resuspended in Newborn Calf Serum (Sigma) and incubated for 20 min. at room temperature. The cells were then incubated for 1 h at 4° C. in pre-immune or immune sera, diluted 1:200 in dilution buffer (PBS, 20% Newborn Calf Serum, 0.1% BSA). After centrifugation and washing in PBS/0.1% BSA, samples were incubated for 1 h at 4° C. with R-Phycoerythrin conjugated F(ab)2 goat anti-mouse IgG (Jackson ImmunoResearch Laboratories; Inc.), diluted 1:100. After washing, cells were resuspended in PBS. Samples were acquired by a FACS Calibur apparatus (Becton Dickinson, Mountain View, Calif.) and data were analyzed using the Cell Quest Software (Becton Dickinson). In the case of API-1, to avoid cross-reactive binding of polyclonal sera, a pool of four monoclonal antibodies raised against the protein was used instead of mouse immune serum; a pool of two unrelated monoclonal antibodies was used as a control. To analyze the surface exposure of the PI-2a backbone protein, antisera specific for the 2603, 515 and CJB111 variants were used. Data are expressed as the difference in fluorescence between cells stained with immune sera versus pre-immune sera.

Active mouse maternal immunization. A maternal immunization/neonatal pup challenge model of GBS infection was used to verify the protective efficacy of the antigens in mice, as previously described (11). In brief, CD-1 female mice (6-8 weeks old) were immunized on days 1 (in CFA), 21 and 35 (IFA) with either PBS or 20 μg of protein per dose when immunized with a single antigen or 60 μg of protein per dose (20 μg of each antigen) when immunized with a combination of antigens. Mice were bred 3 days after the last immunization. Within 48 h of birth, pups were injected intraperitoneally with a dose of GBS bacteria calculated to cause 90% lethality. Survival of pups was monitored for 2 days after challenge. Protection was calculated as (percentage deadControl minus percentage deadVaccine) divided by percentage deadControl multiplied by 100. Statistical analysis was performed using Fisher's exact test.

Genomic islands coding for pilus-like structures are always present in clinical isolates of GBS. A total of 289 isolates of invasive and colonizing GBS collected at three centers (CDC, BCM and Istituto Superiore di Sanità, Italy) (Table I) were analyzed for the presence, sequence variability and surface exposure of the three structural components of GBS pili. Two loci have been identified in the genome of GBS strains that can harbor pilus encoding islands. Genes associated with the first locus, pilus island 1 (PI-1), are conserved and present in six of the eight GBS genomes sequenced (12).The second locus is occupied by either of two variants of pilus island 2, PI-2a and PI-2b, that show only limited similarity at the sequence and gene organization levels (13).

Screening by PCR for the presence of the genes coding for the structural components of GBS pili indicated that all 289 strains contained at least one of the pilus island regions (Table II). The PI-1 locus was present in 208 (72%) strains and was always associated with the presence of a PI-2a or PI-2b allele at the second genomic region containing a pilus island. Therefore, while the genomic region at the first locus was empty in 28% of the strains, the second locus contained PI-2a/b in all GBS isolates. It should be noted that PI-2a was frequently present alone while PI-2b only rarely was not associated with PI-1. However, the most frequent combination was PI-1+PI-2a since it was present in over 45% of isolates.

Table II summarizes the distribution of pilus islands among the GBS isolates grouped by invasive disease manifestation or asymptomatic colonization. The data show no apparent association of disease or colonization with the presence of PI-1 and/or PI-2a/b, albeit few colonizing strains were studied. Similarly, no significant difference in the distribution of the islands was found between these three groups of isolates collected from patients in different geographic areas (data not shown). Thus, all strains were combined in the additional analyses reported.

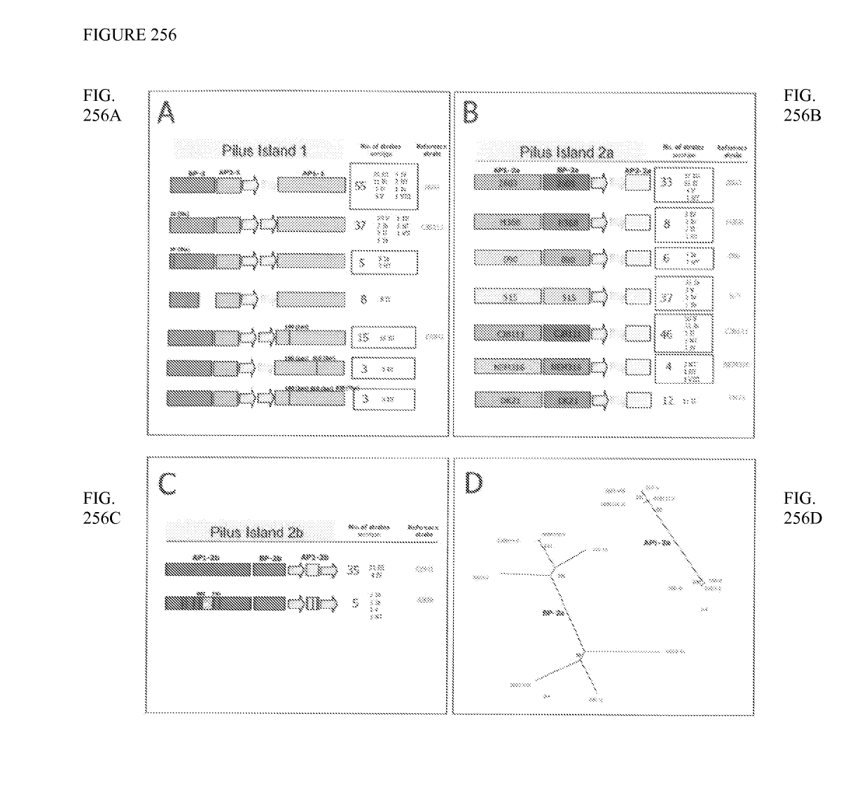

Pilus Islands distribution correlates with serotype. Extending the analysis of pilus island distribution to GBS strains grouped by serotype, a good correlation was observed between presence of a particular combination of PIs and CPS type. Most serotype IA isolates (91%) contained only the PI-2a island, while the large majority (85%) of type IB strains had PI-1 inserted in their genome, as well as PI-2a (FIG. 256). Serotype II strains were always associated with PI-2a, alone or together with PI-1 in a nearly 50:50 ratio. In contrast, all serotype III isolates except one contained PI-1 in association with PI-2a (30%) or, more frequently, with PI-2b (69%). Indeed, 71 of 76 (94%) strains containing PI-1+PI-2b were serotype III.

Only 10 serotype IV isolates were included in this study and the distribution of pilus islands among these few strains does not correlate with the presence of a specific PI, except that, of 289 isolates, the only four strains that contained PI-2b alone were serotype IV (FIG. 256). For serotype V strains, nearly all (96%) contained PI-1+PI-2a and the remaining 4% had only PI-2a. Similarly, PI-1 and PI-2a were both present in the majority of NT strains.

Sequence conservation of PIs structural components. The PCR products obtained amplifying with specific primers the genomic regions coding for the three structural pili components of each island were sequenced for a total of 186 isolates, namely all the strains from the CDC and the Istituto Superiore di Sanità collections.

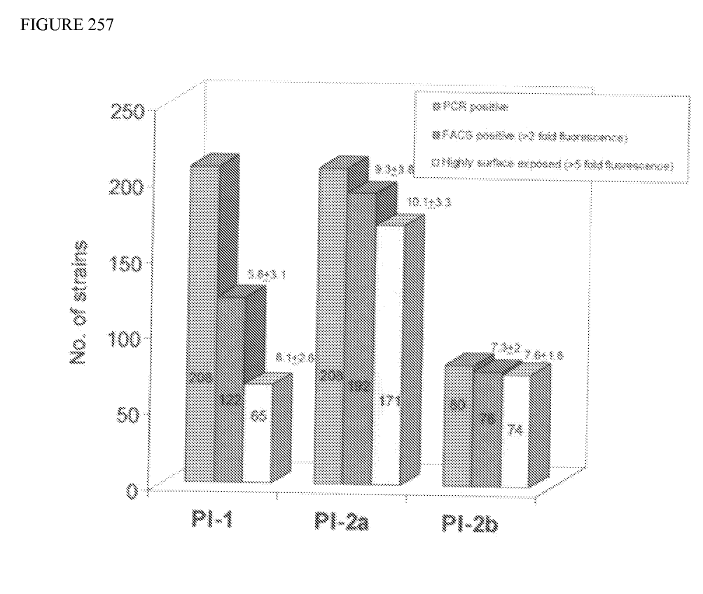

A summary of this analysis is presented in FIG. 257. The three genes coding for the structural proteins of PI-1 were extremely well conserved and their products differed by very few amino acids. In particular, the sequence of BP-1, the protein that is presumed to represent the backbone of pilus 1, showed a polymorphism at position 16 of the signal peptide sequence where a Met residue is substituted by Ile. This was observed in 37 strains, mostly serotype V isolates (78%). Smaller groups of strains contained single amino acid variants at other positions, such as the five isolates that had an Ala57Thr substitution or the eight strains, all serotype II isolates, that carried a point mutation resulting in a frameshift producing a termination codon after the Thr360 codon (FIG. 257, panel A). Similarly, one of the alleles of AP1-1, the major ancillary protein of pilus 1, contained an Ile190Asn polymorphism and all the strains that contained Asn at position 190 belonged to the serotype III group.

FIG. 256, panel B displays a schematic representation of the distribution of alleles of pilus island 2a among the 137 GBS isolates that contained this island. Since two of the structural proteins of PI-2a, BP-2a and AP1-2a, are variable and variants corresponding to five of the strains whose genome has been fully sequenced already have been described (12), the sequences analyzed here have been assigned to the variants present in these reference strains. Furthermore, two additional alleles have been identified among our isolates that are identical to those found in two laboratory strains, 090 (14) and DK21. Therefore, these were arbitrarily chosen as additional reference strains.

The phylogenetic relationship of BP-2a and AP1-2a variants in reference strains used in this study is shown in panel D of FIG. 257. Variability among AP1-2a alleles was limited and sequences from all reference strains fell into two groups that could be identified as the 2603 V/R and the H36B groups, which displayed 87% of amino acidic identity. In contrast, BP-2a variants were clearly more distant and only two alleles, the CJB111 and NEM316 variants that differ by 17 nucleotides or 11 amino acids, can be considered to be highly similar.

All the isolates analyzed contained BP-2a and AP1-2a variants from the same reference strain. Moreover, the distribution of PI-2a variants was strongly biased and correlated with strain serotype as well as with the presence/absence of PI-1 in the same strain. As shown in FIG. 257, serotype IA strains were predominantly associated with the 515 variant for the backbone protein BP-2a and the ancillary protein AP1-2a. Interestingly, all 31 strains containing the 515 variant belonged to the group of serotype IA isolates that had PI-2a alone, while the remaining 3 serotype IA strains, which were assigned to variant 090, contained PI-2a always in association with PI-1 (FIG. 256).

Similarly, the presence of PI-2a variant DK21 was restricted to serotype II strains devoid of PI-1 and, interestingly, this allele was found exclusively in serotype II strains from the CDC collection. Serotype II strains carrying PI-1 as well as PI-2a were associated with variants CJB111 or 2603 V/R. The same was observed for serotype III isolates. These nearly always contained PI-1 together with variant 2603 V/R, since this allele was never found in strains with PI-2a alone. This also was seen for variant CJB111 which was found only in strains containing PI-1 together with PI-2a, particularly in serotype IB and V strains.

Sequence analysis of PI-2b in 40 isolates has shown that the structural components of this island are very conserved. In particular, in 35 isolates, all of serotype III or IV, the sequences coding for the PI-2b pilus were 100% identical to those of strain COH1, whereas in the remaining 5 isolates, not belonging to serogroups III or IV, the sequence of the two genes coding for the ancillary proteins (AP1-2b and AP2-2b) were 100% identical to the corresponding sequences of the A909 reference strain. It is noteworthy that, as was found for the two reference strains, the gene coding for the pilus backbone (BP-2b) shared 100% identity in all 40 isolates (FIG. 257, panel C).

Pilus Islands components are surface exposed. Surface exposure of pili components was assessed by flow cytometry (FACS) analysis using antisera specific for the backbone and the major ancillary protein of each PI in intact cells of all 289 GBS isolates. The outcome of this analysis was instrumental in determining the relative amount of pilus component exposed on the bacterial surface and, more importantly, for assessing how suitable an antigen would be in protection against invasive GBS strains. In fact, it has been established that a 5-fold or greater shift in fluorescence over that observed in the control, stained with preimmune sera, correlates well with protective immunity (11).

FIG. 258 summarizes the results obtained after staining of all GBS isolates with antisera specific for BP-1 and AP1-1 (PI-1), BP-2a and AP1-2a (PI-2a), BP-2b and AP1-2b (PI-2b). Data are presented showing shifts in fluorescence greater than 2-fold or 5-fold the negative control. The lower threshold enables determination of the presence of the antigen on the bacterial surface, while the higher threshold gives a good correlation with in vivo protection (10). Surface staining with sera specific for PI-1 demonstrate that only 59% of the strains containing the island were FACS positive and only approximately half of these exposed the PI-1 antigens at high levels (FIG. 258). In contrast, the structural components of both PI-2a and PI-2b were surface exposed in over 90% of the strains and most of these showed a greater than 5-fold shift in fluorescence. In fact, high surface exposure was observed in 82% of the strains containing PI-2a and 92.5% of those with PI-2b. Since more than 70% of the isolates contained two pilus islands, it was important to establish if high surface exposure of components of two pilus types occurred simultaneously in the same strain. The results, presented in Table III, highlight how PI-1 antigens can be highly exposed on the surface in strains which contain, but do not expose, proteins of PI-2a or PI-2b. It is noteworthy that serotype V isolates demonstrated high surface exposure exclusively of PI-1 antigens in only 15 of 38 strains. In conclusion, a total of 263 isolates, representing 91% of all strains analyzed in this work, expose on the bacterial surface at least one of the three PIs at high level.

Each of the three types of pili contains two protective antigens. We have previously demonstrated that pilus components encoded by both PI-1 (BP-1 and AP1-1) and PI-2a (BP-2a and AP1-2a) are able to induce protective immunity in mice against GBS infection and that the levels of protection strongly correlate with antigen surface exposure (11). To investigate if structural components of PI-2b also elicit protection in vivo, we analyzed the recombinant proteins BP-2b and AP1-2b, expressed in E. coli as His-tagged fusions, by the active murine maternal immunization-neonatal pup challenge model previously described (11). CD-1 female mice were immunized with three doses (days 1, 21, 35) of either 20 μg of each antigen or PBS mixed with Freund's adjuvant, then mated and the resulting offspring were infected with a lethal dose of different GBS strains. As reported in Table IV, both proteins conferred significant levels of protection against those challenge strains in which the antigens were present and highly exposed on the bacterial surface (>5-fold shift in fluorescence).

AP1-2a variants from Pilus Island 2a are cross-protective. Since antigens encoded from PI-2a (BP-2a and AP1-2a, respectively the backbone and the ancillary protein 1) are the only protective pilin proteins showing gene variability, we investigated whether the allelic variants identified were protective not only against strains expressing a homologous protein but also against strains expressing a different variant.

We overexpressed the AP1-2a variants (2603 and H36B) and three of the six BP-2a variants (2603, 515 and CJB111) that together represent more than 80% of the sequenced genes coding for BP-2a. Each soluble purified protein was assessed in the mouse model described above using as challenge strains expressing either a homologous or a heterologous variant. As reported in Table V, all BP-2a proteins analyzed were able to protect only pups challenged with strains carrying the allelic variant used to immunize their mothers, while protection was not observed against strains expressing a heterologous allele. We also tested the in vitro opsonophagocytic activity of sera from mice immunized with the single variants in the presence of human polymorphonuclear leukocytes (PMNs) and baby rabbit complement by using different GBS strains each expressing one allelic variant. The results obtained uniformly correlated with the protection data reported above. In fact, all sera promoted efficient, complement-dependent opsonophagocytosis and killing by PMNs of only those strains carrying the homologous allele (data not shown). Both GBS 67 variants were cross-protective (Table V), and able to protect the offspring of immunized mice against lethal challenge with strains expressing either homologous or heterologous variants and antisera specific for each allele were able to mediate killing of bacteria expressing both variants (data not shown).

A pilus-based vaccine against GBS infections. We previously have demonstrated that a combination of protective antigens not effective against all strains (either not present or not sufficiently exposed on the bacterial surface) can be useful to develop a broadly effective vaccine against GBS infections (11). Although the six pilus antigens identified so far (two for each pilus type, the backbone and the major ancillary protein) are not universally protective antigens, a combination of all three pili can confer broad protection as demonstrated by this example.

In order to obtain the best minimal protein component vaccine formulation, we selected 3 antigens, one protein for each pilus type: the backbone components from PI-1 (BP-1) and PI-2b (BP-2b) and the ancillary protein 1 from PI-2a (AP1-2a). In fact, as our antigens are co-expressed in pairs in the same strains the exclusion of one protein for each pilus should not impact the vaccine coverage, but would reduce vaccine complexity. Selection criteria were based on gene variability results, on levels of protection in vivo compared with opsonophagocytic activity of each antigen in vitro and, finally, on difficulties of expression and purification. Although BP-2a is the main component (the backbone) of pilus type 2a and a very high opsonophagocytic activity was observed in vitro with sera of mice immunized with this single protein, we excluded this antigen due to its high gene variability and because its variants were not cross-protective against each other. For the pilus type 1 and type 2b, we excluded AP1-1 and AP1-2b mainly on the basis of the lower levels of protection observed in mice with respect to the corresponding alternative protein. The combination of the three selected antigens then was assessed in vivo in the same maternal/neonatal mouse model using a panel of GBS strains each expressing at high levels on the bacterial surface different combinations of pilus-like structures. As reported in Table VI, we observed protection against all strains tested with levels ranging from 50% to 100%. On the basis of the surface expression data of the three antigens in the collection of 289 isolates analyzed in this study and considering that at least one antigen was highly surface exposed (>5-fold shift in fluorescence), we estimate the strain coverage of a potential pilus-based vaccine would exceed 91% of the circulating strains assuming that these strains are representative of all invasive GBS strains.

Discussion. It has been shown that structural components of pili induce protective immunity in mouse models of GBS (11, 13). To date, three genomic islands coding for pilus-like structures have been identified in GBS (12, 13). However, as these islands are not conserved in all strains, a thorough study of their distribution was necessary to verify their potential as vaccine candidates. In this Example, we have analyzed a large number of GBS clinical isolates, mainly from infants and adults invasive infections, in order to assess the distribution of the three pilus-like genomic islands.

These represent regions of genomic diversity both in terms of presence/absence in the genome of a given GBS strain as well as for the sequence variability found between the same pilus components in different strains (12). An important finding in our analysis of 289 isolates collected in distant geographic areas was that all contained at least one of the three pilus genomic islands demonstrating that a vaccine with at least one antigen from each pilus island will provide broad protection. Furthermore, the locus harboring the PI-2 alleles was never found empty, with PI-2a being the predominant allele (present in 72% of the strains). This indicates that attribution of the pilus genomic island PI-2 to the “dispensable genome” of GBS (12, 15) should be re-defined. The finding that PI-2 alleles, different in structure but specifying similar functions (assembly of a pilus), are always present at the same locus, which as a consequence is never empty, suggests that this island represents a “variable” component of the core genome of GBS rather than a “dispensable” part of it. Alternatively, the presence of either of the two PI-2 pilus structures is so critical to the pathogenesis of invasive GBS disease that we could not find a single clinical isolate devoid of PI-2. This further indicates that a vaccine with an antigen selected from each PI-2 variant will similarly have broad protective scope.

As pili may be important for adhesion and host colonization, a first aim of our study was to verify if there was a correlation between the pilus islands genetic composition in clinical isolates and the type of invasive disease. No apparent association was found between the presence/absence of a particular PI and type of disease or carriage. This is in agreement with previous reports addressing the same question with regard to different genetic traits of GBS isolates, such as capsular serotypes and phylogenetic lineages. In general, reports in the literature indicate that there is no strong association between capsular loci and type of disease (16). However, there has been consistent and sustained epidemiologic evidence that serotype III strains are strongly associated with early- and late-onset meningitis as well as with late-onset infection irrespective of focus (5, 17) and, in particular, that specific lineages of serotype III GBS strains possibly correlate with early-onset disease (18, 19). Studies on distribution of several virulence factors (20) or pathogenicity islands (21) also failed to establish an absolute correlation between the presence in GBS isolates of a particular genetic determinant and the age at onset or clinical manifestation of disease.

In this example, the presence of a particular pilus island allele in a clinical isolate correlates well only with the CPS serotype of the strain. Generally, PI-1 is rarely present in serotype Ia strains, which contain predominantly only PI-2a, and is almost exclusively associated with PI-2a in serotype Ib and V strains. The presence of PI-2b alleles is restricted mainly to serotype III and IV isolates. Thus, in designing a vaccine, antigens from PI-2b are interchangeable with capsular polysaccharides from group II and IV. Interestingly, the few cases that do not display this correlation always contain variants of the PI sequences not conserved with respect to those found in the other strains. This is particularly true for the PI-2b genomic island, whereas for the PI-2a allele, which shows the broadest distribution and the highest degree of sequence divergence between the different strains, variants of the BP-2a and AP1-2a pilus components correlate both with strain serotype and presence in the same strain of the PI-1 pilus. As an example, when PI-2a is present in conjunction with PI-1 in serotype Ia strains, the sequences of BP-2a and AP1-2a are identical to the 090 variant, while the presence of PI-2a alone is always associated with the 515 variants of these genes. Thus, strains that differ in their PI composition with respect to the strains of the same serotype, invariably contain PI alleles different from those present in the other strains of that serotype.

In the context of this work, the analysis of distribution and gene variability of pili in clinical isolates of GBS had the objective of defining the coverage that a pilus-based vaccine against GBS would give by using as antigens pili components that are highly conserved in a wide range of isolates. A prerequisite of antigens used to induce protective immunity is that they should be well exposed on the bacterial surface. In fact, the levels of protection against GBS infection in murine models strongly correlate with antigen surface exposure (11). Among the 289 clinical isolates analyzed, a relatively low percentage of strains harboring PI-1 (31%) show high surface exposure of the PI-1 pilus components, whereas most of the strains containing PI-2a (82%) or PI-2b (92.5%) expose high levels of pilus proteins on the surface. The reason for this difference in behavior is unclear and more studies are needed to clarify this point. Certainly, the low percentage of strains exposing the PI-1 pilus on the surface cannot be ascribed to sequence diversity of PI-1 components in different strains, since this island shows the highest sequence conservation. It is more likely that, in our experimental conditions, expression and assembly of pili components are regulated in a different manner for the different types of pili. Whatever the reason for this, it is very important to note that 15 serotype V clinical isolates, containing both PI-1 and PI-2a, and 3 serotype III strains, which contain PI-1 in conjunction with PI-2b, demonstrated high surface exposure only of PI-1 components. Thus, inclusion of the PI-1 backbone protein in a GBS vaccine should induce protection against a significant number of serotype V GBS infections and therefore are interchangeable with capsular polysaccharides from serotype V. The coverage by including the PI-2b backbone protein in a vaccine combination is, instead, substantial for serotype III GBS strains.

### References for Example 28

1. Telford, J. L., M. A. Barocchi, I. Margarit, R. Rappuoli, and G. Grandi. 2006. Pili in gram-positive pathogens. Nat Rev Microbiol 4:509-519.

2. Beckmann, C., J. D. Waggoner, T. O. Harris, G. S. Tamura, and C. E. Rubens. 2002. Identification of novel adhesins from Group B streptococci by use of phage display reveals that C5a peptidase mediates fibronectin binding. Infect Immun 70:2869-2876.

3. Tamura, G. S., and C. E. Rubens. 1995. Group B streptococci adhere to a variant of fibronectin attached to a solid phase. Mol Microbiol 15:581-589.

4. Spellerberg, B., E. Rozdzinski, S. Martin, J. Weber-Heynemann, N. Schnitzler, R. Lutticken, and A. Podbielski. 1999. Lmb, a protein with similarities to the LraI adhesin family, mediates attachment of Streptococcus agalactiae to human laminin. Infect Immun 67:871-878.

5. Gibbs, R. S., S. Schrag, and A. Schuchat. 2004. Perinatal infections due to group B streptococci. Obstet Gynecol 104:1062-1076.

6. Edwards, M. S., and C. J. Baker. 2005. Group B streptococcal infections in elderly adults. Clin Infect Dis 41:839-847.

7. Benson, J. A., A. E. Flores, C. J. Baker, S. L. Hillier, and P. Ferrieri. 2002. Improved methods for typing nontypeable isolates of group B streptococci. Int J Med Microbiol 292:37-42.

8. Ramaswamy, S. V., P. Ferrieri, A. E. Flores, and L. C. Paoletti. 2006. Molecular characterization of nontypeable group B streptococcus. J Clin Microbiol 44:2398-2403.

9. Harrison, L. H., J. A. Elliott, D. M. Dwyer, J. P. Libonati, P. Ferrieri, L. Billmann, and A. Schuchat. 1998. Serotype distribution of invasive group B streptococcal isolates in Maryland: implications for vaccine formulation. Maryland Emerging Infections Program. J Infect Dis 177:998-1002.

10. Baker, C. J., D. L. Kasper, I. Tager, A. Paredes, S. Alpert, W. M. McCormack, and D. Goroff. 1977. Quantitative determination of antibody to capsular polysaccharide in infection with type III strains of group B Streptococcus. J Clin Invest 59:810-818.

11. Maione, D., I. Margarit, C. D. Rinaudo, V. Masignani, M. Mora, M. Scarselli, H. Tettelin, C. Brettoni, E. T. Iacobini, R. Rosini, N. D'Agostino, L. Miorin, S. Buccato, M. Mariani, G. Galli, R. Nogarotto, V. N. Dei, F. Vegni, C. Fraser, G. Mancuso, G. Teti, L. C. Madoff, L. C. Paoletti, R. Rappuoli, D. L. Kasper, J. L. Telford, and G. Grandi. 2005. Identification of a Universal Group B Streptococcus Vaccine by Multiple Genome Screen. Science 309:148-150.

12. Tettelin, H., V. Masignani, M. J. Cieslewicz, C. Donati, D. Medini, N. L. Ward, S. V. Angiuoli, J. Crabtree, A. L. Jones, A. S. Durkin, R. T. Deboy, T. M. Davidsen, M. Mora, M. Scarselli, Y. R. I. Margarit, J. D. Peterson, C. R. Hauser, J. P. Sundaram, W. C. Nelson, R. Madupu, L. M. Brinkac, R. J. Dodson, M. J. Rosovitz, S. A. Sullivan, S. C. Daugherty, D. H. Haft, J. Selengut, M. L. Gwinn, L. Zhou, N. Zafar, H. Khouri, D. Radune, G. Dimitrov, K. Watkins, J. O'Connor K, S. Smith, T. R. Utterback, O. White, C. E. Rubens, G. Grandi, L. C. Madoff, D. L. Kasper, J. L. Telford, M. R. Wessels, R. Rappuoli, and C. M. Fraser. 2005. Genome analysis of multiple pathogenic isolates of Streptococcus agalactiae: Implications for the microbial “pan-genome”. Proc Natl Acad Sci USA 102:13950-13955.

13. Rosini, R., C. D. Rinaudo, M. Soriani, P. Lauer, M. Mora, D. Maione, A. Taddei, I. Santi, C. Ghezzo, C. Brettoni, S. Buccato, I. Margarit, G. Grandi, and J. L. Telford. 2006. Identification of novel genomic islands coding for antigenic pilus-like structures in Streptococcus agalactiae. Mol Microbiol 61:126-141.

14. Lancefield, R. C. 1938. Two serological types of group B hemolytic streptococci with related, but not identical, type-specific substances. J Exp Med 67:25-40.

15. Medini, D., C. Donati, H. Tettelin, V. Masignani, and R. Rappuoli. 2005. The microbial pan-genome. Curr Opin Genet Dev 15:589-594.

16. Hauge, M., C. Jespersgaard, K. Poulsen, and M. Kilian. 1996. Population structure of Streptococcus agalactiae reveals an association between specific evolutionary lineages and putative virulence factors but not disease. Infect Immun 64:919-925.

17. Weisner, A. M., A. P. Johnson, T. L. Lamagni, E. Arnold, M. Warner, P. T. Heath, and A. Efstratiou. 2004. Characterization of group B streptococci recovered from infants with invasive disease in England and Wales. Clin Infect Dis 38:1203-1208.

18. Jones, N., J. F. Bohnsack, S. Takahashi, K. A. Oliver, M.-S. Chan, F. Kunst, P. Glaser, C. Rusniok, D. W. M. Crook, R. M. Harding, N. Bisharat, and B. G. Spratt. 2003. Multilocus Sequence Typing System for Group B Streptococcus. J. Clin. Microbiol. 41:2530-2536.

19. Lin, F.-Y. C., A. Whiting, E Adderson, S. Takahashi, D. M. Dunn, R. Weiss, P. H. Azimi, J. B. Philips, III, L. E. Weisman, J. Regan, P. Clark, G. G. Rhoads, C. E. Frasch, J. Troendle, P. Moyer, and J. F. Bohnsack. 2006. Phylogenetic Lineages of Invasive and Colonizing Strains of Serotype III Group B Streptococci from Neonates: a Multicenter Prospective Study. J. Clin. Microbiol. 44:1257-1261.

20. Brochet, M., E. Couve, M. Zouine, T. Vallaeys, C. Rusniok, M. C. Lamy, C. Buchrieser, P. Trieu-Cuot, F. Kunst, C. Poyart, and P. Glaser. 2006. Genomic diversity and evolution within the species Streptococcus agalactiae. Microbes Infect 8:1227-1243.

21. Herbert, M. A., C. J. Beveridge, D. McCormick, E. Aten, N. Jones, L. A. Snyder, and N. J. Saunders. 2005. Genetic islands of Streptococcus agalactiae strains NEM316 and 2603VR and their presence in other Group B streptococcal strains. BMC Microbiol 5:31.

22. Montigiani, S., F. Falugi, M. Scarselli, O. Finco, R. Petracca, G. Galli, M. Mariani, R. Manetti, M. Agnusdei, R. Cevenini, M. Donati, R. Nogarotto, N. Norais, I. Garaguso, S. Nuti, G. Saletti, D. Rosa, G. Ratti, and G. Grandi. 2002. Genomic approach for analysis of surface proteins in Chlamydia pneumoniae. Infect Immun 70:368-379.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be covered by the present invention.

TABLE I
GBS clinical isolates analyzed in this study.

| | | Serotype | | | | | | | | |
|---|---|---|---|---|---|---|---|---|---|---|
| Origin | Disease | IA | IB | II | III | IV | V | Other | NT | Total |
| CDC-Center for Disease | Early onset | 13 | 3 | 9 | 13 | 1 | 14 | | | 53 |
| Control, Atlanta, USA | Late onset | 4 | 4 | 4 | 6 | | 6 | | | 24 |
| | Adult | 4 | 1 | 5 | 5 | 4 | 4 | | | 23 |
| | | 21 | 8 | 18 | 24 | 5 | 24 | | | 100 |
| Baylor College of | Early onset | 9 | 2 | 2 | 12 | | 5 | | 1 | 31 |
| Medicine, Houston, USA | Late onset | 12 | 3 | | 41 | | 6 | | | 62 |
| | Adult | | | | | | | | 10* | 10 |
| | | 21 | 5 | 2 | 53 | | 11 | | 11 | 103 |
| Istituto Superiore di | Early onset | 5 | 1 | 2 | 6 | 1 | 3 | | | 18 |
| Sanita, Rome, Italy | Late onset | 1 | 2 | | 11 | | 2 | | | 16 |
| | Adult | 9 | 3 | 6 | 8 | 2 | 7 | 2 | 6 | 43 |
| | Colonizing | 1 | 2 | 2 | | 3 | 1 | | | 9 |
| | | 16 | 8 | 10 | 25 | 6 | 13 | 2 | 6 | 86 |

*NT by serotyping; using genotyping among these 10 strains there were 3 IA, 2 IV, 4 V and 1 VI types.

TABLE II
Pilus islands distribution in 289 isolates of invasive and colonizing GBS.

| | Early onset | Late onset | Adult | Colonizing | |
|---|---|---|---|---|---|
| | No. of strains (%) | | | | All isolates (%) |
| PI-1 | | | | | 0 |
| PI-2a | 33 (32) | 21 (21) | 20 (26) | 3 (33) | 77 (26.6) |
| PI-2b | 1 (1) | | 3 (4) | | 4 (1.4) |
| PI-1 + PI-2a | 45 (44) | 36 (35) | 46 (61) | 5 (56) | 132 (45.7) |
| PI-1 + PI-2b | 23 (23) | 45 (44) | 7 (9) | 1 (11) | 76 (26.3) |
| Total | 102 (100) | 102 (100) | 76 (100) | 9 (100) | 289 (100) |

TABLE III
High surface exposure of pilus islands components in GBS clinical isolates grouped by serotype.

| | | | | | | | | | | All |
|---|---|---|---|---|---|---|---|---|---|---|
| | Highly surface | Serotype | | | | | | | | capsular |
| | exposed pilus | Ia | Ib | II | III | IV | V | Other | NT | serotypes |
| PI-2a | | 49 (92) | 1 (5) | 13 (54) | 1 (1) | 1 (10) | 2 (4) | 1 (33) | 4 (24) | 72 (27) |
| PI-2b | | | | | | 4 (40) | | | | 4 (2) |
| | PI-1 | | | | | | 15 | | | |
| | PI-2a | 4 | 16 | 10 | 24 | 4 | 6 | 1 | 8 | |
| | PI-1 + PI-2a | | | 1 | 4 | | 17 | | 4 | |
| PI-1 + PI-2a | total | 4 (8) | 16 (84) | 11 (46) | 28 (29) | 4 (40) | 38 (96) | 1 (33) | 12 (71) | 114 (43) |
| | PI-1 | | | | 3 | | | | | |
| | PI-2b | | 1 | | 46 | | | | | |
| | PI-1 + PI-2b | | 1 | | 20 | | | 1 | 1 | |
| PI-1 + PI-2b | total | | 2 (11) | | 69 (70) | | | 1 (33) | 1 (5) | 73 (28) |
| Total | | 53 (100) | 19 (100) | 24 (100) | 98 (100) | 9 (100) | 40 (100) | 3 (100) | 17 (100) | 263 (100) |

NOTE.
Data are number of strains (%)

TABLE IV
Protection conferred by the PI-2b proteins, BP-2b and
AP1-2b, against GBS strains

| | | Fluorescence† | | Statistical |
|---|---|---|---|---|
| | | (immune/ | | significance |
| Antigen | GBS strain (type) | preimmune) | Protection % | p value |
| BP-2b | COH1 (III) | 7.7 | 72.4 | <0.0001 |
| | M781 (III) | 6.2 | 95.8 | <0.0001 |
| | M732 (III) | 9.7 | 87.7 | <0.0001 |
| | 6213 (III) | 8.2 | 72.0 | <0.0001 |
| | SMU071 (VIII) | 11.6 | 56.0 | <0.0001 |
| | A909 (Ia) | 11.5 | 82.9 | <0.0001 |
| | CJB111 (V) | 0* | 17.5 | >0.05 |
| AP1-2b | COH1 (III) | 6.3 | 49.5 | <0.0001 |
| | M781 (III) | 6.3 | 82.1 | <0.0001 |
| | SMU071 (VIII) | 10.2 | 58.0^{§} | <0.0001 |

assessed by active maternal immunization/neonatal pup challenge model.
†expressed as shift in fluorescence
*gene missing in this strain
^{§}N-terminal region of AP1-2b

TABLE V
Protection conferred by BP-2a and AP1-2a variants against strains carrying either
homologous or heterologous proteins tested in the mouse active maternal
immunization/neonatal model.

| | | | | | | Statistical |
|---|---|---|---|---|---|---|
| | GBS strain | Allelic | Protein | PBS | | significance |
| Antigen | (serotype) | variant | alive/treated | alive/treated | Protection % | p value |
| BP-2a | CJB111 (V) | CJB111 | 41/50 | 6/72 | 80.4 | <0.0001 |
| CJB111 variant | 515 (Ia) | 515 | 0/40 | 6/53 | 0 | 0.04 |
| | 3050 (II) | 2603 | 5/39 | 5/46 | 2.2 | 1 |
| BP-2a | CJB111 (V) | CJB111 | 6/40 | 6/72 | 7.6 | >0.05 |
| 515 variant | 515 (Ia) | 515 | 24/43 | 6/53 | 50.6 | <0.0001 |
| | 3050 (II) | 2603 | 3/34 | 5/46 | 0 | 1 |
| BP-2a | CJB111 (V) | CJB111 | 0/20 | 6/72 | 0 | >0.05 |
| 2603 variant | 515 (Ia) | 515 | 14/37 | 6/53 | 30 | 0.004 |
| | 3050 (II) | 2603 | 28/30 | 5/46 | 92.1 | <0.0001 |
| AP1-2a | CJB111 (V) | CJB111 | 31/40 | 9/35 | 69.6 | <0.0001 |
| 2603 variant | 515 (Ia) | 515 | 31/40 | 14/34 | 61.9 | 0.0018 |
| | 3050 (II) | 2603 | 36/38 | 5/46 | 94.4 | <0.0001 |
| | 5401 (II) | H36B | 37/54 | 11/76 | 62.8 | <0.0001 |
| AP1-2a | 515 (Ia) | 515 | 45/75 | 5/80 | 57.4 | <0.0001 |
| H36B variant | 5401 (II) | H36B | 31/45 | 10/40 | 58.7 | <0.0001 |
| | DK21 (II) | H36B | 22/33 | 6/36 | 60.2 | <0.0001 |

TABLE VI
Neonatal protection conferred by a pilus-based vaccine formulation against
nine strains expressing different combinations of pili. Mice were immunized
with a mixture of 15 μg of each pilus protein (BP-1 + AP1-2a + BP-2b)
in Freund's adjuvant.

| | | Fluorescence (Immune-Preimmune) | | | | | | |
|---|---|---|---|---|---|---|---|---|
| | | | BP-1 | AP1-2a | BP-2b | Vaccine | PBS | Protection* |
| GBS strains | Serotype | PCR | (PI-1) | (PI-2a) | (PI-2b) | Alive/Treated | Alive/Treated | % |
| A909 | Ia | PI-1 + PI-2b | 0 | — | 10.6 | 35/40 | 2/40 | 86.3 |
| 515 | Ia | PI-2a | — | 10.1 | — | 28/53 | 0/40 | 53.0 |
| 3050 | II | PI-1 + PI-2a | 1.7 | 11.3 | — | 68/68 | 14/49 | 100 |
| 5401 | II | PI-1 + PI-2a | 1.8 | 15.2 | — | 31/39 | 11/50 | 73.1 |
| COH1 | III | PI-1 + PI-2b | 7.5 | — | 7.7 | 52/54 | 5/60 | 95.7 |
| M732 | III | PI-1 + PI-2b | 3.5 | — | 9.7 | 37/40 | 16/57 | 88.9 |
| M781 | III | PI-1 + PI-2b | 2.7 | — | 6.2 | 30/48 | 11/50 | 51.3 |
| CJB111 | V | PI-1 + PI-2a | 9.0 | 11.8 | — | 51/54 | 9/30 | 91.4 |
| JM9130013 | VIII | PI-1 + PI-2b | 14.4 | — | 11.9 | 24/39 | 8/47 | 54.2 |

*Protection p value <0.0001 by Fisher's exact test

TABLE VII
Primers used for PCR amplifications.

| | | |
|---|---|---|
| Primer | Sequence (5′ to 3′) | Description |

| | | |
|---|---|---|
| BP-1 for | CTGTGCTAAACTTTAATAGTTTTGTGC | Primers for amplification of the PI-1 backbone coding gene |
| BP-1 rev | AGGCTATTTCTCTTTTCTTAAACATGG | |
| AP1-1 for | GTCAATAACGATGTTGTGAATGGC | Primers for amplification of the PI-1 ancillary protein 1 |
| AP1-1 rev | CTGACAATAGGGAAAGGCCAAAAC | coding gene |
| AP2-1 for | CCATGTTTAAGAAAAGAGAAATAGCCT | Primers for amplification of the PI-1 ancillary protein 2 |
| AP2-1 rev | GGAAACCCGCTAAGA7˜AATTAAACG | coding gene |
| BP-2a for | CTATGATGTCTATTGCAGGTGGAA | Primers for amplification of the PI-2a backbone coding |
| BP-2a rev | GAGTACTTCTACTGAAATACCC | gene |
| AP1-2a for | CTAGGATATTCTGTATCTGATCTTAG | Primers for amplification of the PI-2a ancillary protein 1 |
| AP1-2a rev | ACCGCAGCACCACTCCATTC | coding gene |
| AP2-2a for | CCTCTTATCAATCCTAGTTATTC | Primers for amplification of the PI-2a ancillary protein 2 |
| AP2-2a rev | CCCTGCCCTGAAGACACCTA | coding gene |
| BP-2b for | TTTAGCTTTAGCTCTACCATCAGG | Primers for amplification of the PI-2b backbone coding |
| BP-2b rev | CCCACTGCAATTATAGTGACAAGC | gene |
| AP1-2b for | TTCTTTCAGAATATGGCCTCTAGG | Primers for amplification of the 5′ half of the PI-2b ancillary |
| AP1N-2b rev | GCCAACCTGCATAGACTTTAACATC | protein 1 coding gene |
| AP1C-2b for | GATCCGCTTGATAATACAGAGCTTC | Primers for amplification of the 3′ half of the PI-2b ancillary |
| AP1-2b rev | TCGCCACTAACAGCGATTGAATC | protein 1 coding gene |
| AP2-2b for | AGAAGTAACTAGGAGAGCACGTC | Primers for amplification of the PI-2b ancillary protein 2 |
| AP2-2b rev | CCCCAATAGATTCCAGAACTTACC | coding gene |

1. An immunogenic composition comprising a purified Gram positive bacteria adhesin island (AI) polypeptide in an oligomeric form.

2. The immunogenic composition of claim 1 wherein the AI polypeptide comprises a sortase substrate motif.

3. The immunogenic composition of claim 2 wherein the sortase substrate motif is an LPXTG motif.

4. The immunogenic composition of claim 3 wherein the LPXTG motif is represented by the sequence XPXTG, wherein X at amino acid position 1 is L, I, or F and wherein X at amino acid position 3 is any amino acid residue.

5. The immunogenic composition of claim 3 wherein the LPXTG motif is represented by XXXXG, wherein X at amino acid position 1 is L, V, E, I, F, or Q; wherein X at amino acid position 2 is P if X at amino acid position 1 is L, I, or F; wherein X at amino acid position 2 is V if X at amino acid position 1 is E or Q; wherein X at amino acid position 2 is V or P if X at amino acid position 1 is V; wherein X at amino acid position 3 is any amino acid residue; wherein X at amino acid position 4 is T if X at amino acid position 1 is V, E, I, F, or Q; and wherein X at amino acid position 4 is T, S, or A if X at amino acid position 1 is L.

6. The immunogenic composition of claim 1 wherein the Gram positive bacteria AI polypeptide affects the ability of Gram positive bacteria to adhere to epithelial cells.

7. The immunogenic composition of claim 1 wherein the Gram positive bacteria AI polypeptide affects the ability of Gram positive bacteria to invade epithelial cells.

8. The immunogenic composition of claim 1 wherein the Gram positive bacteria AI polypeptide affects the ability of Gram positive bacteria to translocate through an epithelial cell layer.

9. The immunogenic composition of claim 1 wherein the Gram positive bacteria AI polypeptide is capable of associating with an epithelial cell surface.

10. The immunogenic composition of claim 9 wherein the associating with an epithelial cell surface is binding to the epithelial cell surface.

11. The immunogenic composition of claim 1 wherein the Gram positive bacteria AI polypeptide is a full-length protein.

12. The immunogenic composition of claim 1 wherein the Gram positive bacteria AI polypeptide is a fragment of a full-length protein.

13. The immunogenic composition of claim 12 wherein the fragment comprises at least 7 contiguous amino acid residues of the Gram positive bacteria AI protein.

14. The immunogenic composition of claim 1 wherein the Gram positive bacteria are of a genus selected from the group consisting of Streptococcus, Enterococcus, Staphylococcus, Clostridium, Corynebacterium, or Listeria.

15. The immunogenic composition of claim 14 wherein the Gram positive bacteria are of the genus Streptococcus.

16. The immunogenic composition of claim 15 wherein the bacteria are Group B Streptococcus (GBS).

17. The immunogenic composition of claim 16 wherein the AI polypeptide is a GBS Adhesin Island 1 (AI-1) polypeptide.

18. The immunogenic composition of claim 17 wherein the GBS AI-1 polypeptide is selected from the group consisting of GBS 80, GBS 104, GBS 52, and fragments thereof.

19. The immunogenic composition of claim 17 wherein the AI-1 polypeptide is GBS 80.

20. The immunogenic composition of claim 14 wherein the AI polypeptide is a GBS Adhesin Island 2 (AI-2) polypeptide.

21. The immunogenic composition of claim 20 wherein the GBS AI-2 polypeptide is selected from the group consisting of GBS 59, GBS 67, GBS 150, 01521, 01523, 01524, and fragments thereof.

22. The immunogenic composition of claim 15 wherein the Gram positive bacteria are Group A Streptococcus (GAS) polypeptide.

23. The immunogenic composition of claim 22 wherein the AI polypeptide is a GAS Adhesin Island 1 (GAS AI-1) polypeptide.

24. The immunogenic composition of claim 23 wherein the GAS AI-1 polypeptide is selected from the group consisting of M6_Spy0157, M6_Spy0159, M6_Spy0160, CDC SS 410_fimbrial, ISS3650_fimbrial, DSM2071_fimbrial, and fragments thereof.

25. The immunogenic composition of claim 22 wherein the polypeptide is a GAS Adhesin Island 2 (GAS AI-2) polypeptide.

26. The immunogenic composition of claim 25 wherein the GAS AI-2 polypeptide is selected from the group consisting of GAS15, GAS16, GAS 18, and fragments thereof.

27. The immunogenic composition of claim 20 wherein the AI polypeptide is a GAS Adhesin Island 3 (GAS AI-3) polypeptide.

28. The immunogenic composition of claim 27 wherein the GAS AI-3 polypeptide is selected from the group consisting of SpyM3_{—}0098, SpyM3_{—}0100, SpyM3_{—}0102, SpyM3_{—}0104, SPs0100, SPs0102, SPs0104, SPs0106, orf78, orf80, orf82, orf84, spyM18_{—}0126, spyM18_{—}0128, spyM18_{—}0130, spyM18_{—}0132, SpyoM01000156, SpyoM01000155, SpyoM01000154, SpyoM01000153, SpyoM01000152, SpyoM01000151, SpyoM01000150, SpyoM01000149, ISS3040_fimbrial, ISS3776_fimbrial, ISS4959_fimbrial, and fragments thereof.

29. The immunogenic composition of claim 22 wherein the AI polypeptide is a GAS Adhesin Island 4 (GAS AI-4) polypeptide.

30. The immunogenic composition of claim 29 wherein the GAS AI-4 polypeptide is selected from the group consisting of 19224134, 19224135, 19224137, 19224139, 19224141, 20010296_fimbrial, 20020069_fimbrial, CDC SS 635_fimbrial, ISS4883_fimbrial, ISS4538_fimbrial, and fragments thereof.

31. The immunogenic composition of claim 22 wherein the AI polypeptide is a GAS Adhesin Island 5 (AI-5) polypeptide.

32. The immunogenic composition of claim 31 wherein the GAS AI-5 polypeptide is selected from the group consisting of MGAS10270_Spy0108, MGAS10270_Spy0109, MGAS10270_Spy0110, MGAS10270_Spy0111, MGAS10270_Spy0112, MGAS10270_Spy0113, MGAS10270_Spy0114, MGAS10270_Spy0115, MGAS10270_Spy0116, and MGAS10270_Spy0117.

33. The immunogenic composition of claim 22 wherein the AI polypeptide is a GAS Adhesin Island 6 (AI-6) polypeptide.

34. The immunogenic composition of claim 33 wherein the GAS AI-6 polypeptide is selected from the group consisting of MGAS10750_Spy0113, MGAS10750_Spy0114, MGAS10750_Spy0115, MGAS10750_Spy0116, MGAS10750_Spy0117, MGAS10750_Spy0118, MGAS10750_Spy0119, and MGAS10750_Spy0120.

35. The immunogenic composition of claim 1 wherein the bacteria are Streptococcus pneumoniae (SP).

36. The immunogenic composition of claim 35 wherein the AI polypeptide is selected from the group consisting of SP0462, SP0463, SP0464, orf3_{—}670, orf4_{—}670, orf5_{—}670, ORF3_{—}14CSR, ORF4_{—}14CSR, ORF5_{—}14CSR, ORF3_{—}19AH, ORF4_{—}19AH, ORF5_{—}19AH, ORF3_{—}19FTW, ORF4_{—}19FTW, ORF5_{—}19FTW, ORF3_{—}23FP, ORF4_{—23}FP, ORF5_{—}23FP, ORF3_{—}23FTW, ORF4_{—}23FTW, ORF5_{—}23FTW, ORF3_{—}6BF, ORF4_{—}6BF, ORF5_{—}6BF, ORF3_{—}136BSP, ORF4_{—}6BSP, ORF5_{—}6BSP, ORF3_{—}9VSP, ORF4_{—}9VSP, ORF5_{—}9VSP, and fragments thereof.

37. The immunogenic composition of claim 1 wherein the oligomeric form is a hyperoligomer.

38. An immunogenic composition comprising a first and a second Gram positive bacteria adhesin island (AI) polypeptide.

39. The immunogenic composition of claim 38 wherein the Gram positive bacteria are of a genus selected from the group consisting of Streptococcus, Enterococcus, Staphylococcus, Clostridium, Corynebacterium, or Listeria.

40. The immunogenic composition of claim 38 wherein the first AI polypeptide is a GBS AI-1 polypeptide.

41. The immunogenic composition of claim 40 wherein the GBS AI-1 polypeptide is selected from the group consisting of GBS 80, GBS 104, GBS 52, and fragments thereof.

42. The immunogenic composition of claim 38 wherein the first AI polypeptide is a GBS AI-2 polypeptide.

43. The immunogenic composition of claim 42 wherein the GBS AI-2 polypeptide is selected from the group consisting of GBS 59, GBS 67, GBS 150, 01521, 01523, 01524, and fragments thereof.

44. The immunogenic composition of claim 38 wherein the first AI polypeptide is GBS 80 and the second AI polypeptide is GBS 67.

45. The immunogenic composition of claim 38 wherein the first AI polypeptide is a Group A Streptococcus (GAS) AI polypeptide.

46. The immunogenic composition of claim 45 wherein the GAS AI polypeptide is a first GAS AI-1 polypeptide.

47. The immunogenic composition of claim 46 wherein the first GAS AI-1 polypeptide is selected from the group consisting of M6_Spy0157, M6_Spy0159, M6_Spy0160, CDC SS 410_fimbrial, ISS3650_fimbrial, DSM2071_fimbrial, and fragments thereof.

48. The immunogenic composition of claim 45 wherein the GAS AI polypeptide is a first GAS AI-2 polypeptide.

49. The immunogenic composition of claim 48 wherein the first GAS AI-2 polypeptide is selected from the group consisting of GAS15, GAS16, GAS 18, and fragments thereof.

50. The immunogenic composition of claim 45 wherein the GAS AI polypeptide is a first GAS AI-3 polypeptide.

51. The immunogenic composition of claim 50 wherein the first GAS AI-3 polypeptide is selected from the group consisting of SpyM3_{—}0098, SpyM3_{—}0100, SpyM3_{—}0102, SpyM3_{—}0104, SPs0100, SPs0102, SPs0104, SPs0106, orf78, orf80, orf82, orf84, spyM18_{—}0126, spyM18_{—}0128, spyM18_{—}0130, spyM18 _{—}0132, SpyoM01000156, SpyoM01000155, SpyoM01000154, SpyoM01000153, SpyoM01000152, SpyoM01000151, SpyoM01000150, SpyoM01000149, ISS3040_fimbrial, ISS3776_fimbrial, ISS4959_fimbrial, and fragments thereof.

52. The immunogenic composition of claim 45 wherein the GAS AI polypeptide is a first GAS AI-4 polypeptide.

53. The immunogenic composition of claim 52 wherein the first GAS AI-4 polypeptide is selected from the group consisting of 19224134, 19224135, 19224137, 19224139, 19224141, 20010296_fimbrial, 20020069_fimbrial, CDC SS 635_fimbrial, ISS4883_fimbrial, ISS4538_fimbrial, and fragments thereof.

54. The immunogenic composition of claim 45 wherein the GAS AI polypeptide is a first GAS AI-5 polypeptide.

55. The immunogenic composition of claim 54 wherein the first GAS AI-5 polypeptide is selected from the group consisting of MGAS10270_Spy0108, MGAS10270_Spy0109, MGAS10270_Spy0110, MGAS10270_Spy0111, MGAS10270_Spy0112, MGAS10270_Spy0113, MGAS10270_Spy0114, MGAS10270_Spy0115, MGAS10270_Spy0116, and MGAS10270_Spy0117.

56. The immunogenic composition of claim 45 wherein the GAS AI polypeptide is a first GAS AI-6 polypeptide.

57. The immunogenic composition of claim 56 wherein the first GAS AI-6 polypeptide is selected from the group consisting of MGAS10750_Spy0113, MGAS10750_Spy0114, MGAS10750_Spy0115, MGAS10750_Spy0116, MGAS10750_Spy0117, MGAS10750_Spy0118, MGAS10750_Spy0119, and MGAS10750_Spy0120.

58. The immunogenic composition of claim 45 wherein the second Gram positive bacteria AI polypeptide is selected from the group consisting of a second GAS AI-1 polypeptide, a second GAS AI-2 polypeptide, a second GAS AI-3 polypeptide, a second GAS AI-4 polypeptide, a second GAS AI-5 polypeptide, and a second GAS AI-6 polypeptide.

59. The immunogenic composition of claim 38 comprising a first and a second S. pneumoniae AI polypeptide.

60. The immunogenic composition of claim 59 wherein the first and the second S. pneumoniae AI polypeptide are each selected from the group consisting of SP0462, SP0463, SP0464, orf3_{—}670, orf4_{—}670, orf5_{—}670, ORF3_{—}14CSR, ORF4_{—}14CSR, ORF1_{—}14CSR, ORF3_{—}19AH, ORF4_{—}19AH, ORF5_{—}19AH, ORF3_{—}19FTW, ORF4_{—}19FTW, ORF5_{—}19FTW, ORF3_{—}23FP, ORF4_{—}23FP, ORF5_{—}23FP, ORF3_{—}23FTW, ORF4_{—}23FTW, ORF5_{—}23FTW, ORF3_{—}6BF, ORF4_{—}6BF, ORF5_{—}6BF, ORF3_{—}6BSP, ORF4_{—}6BSP, ORF5_{—}6BSP, ORF3_{—}9VSP, ORF4_{—}9VSP, ORF5_{—}9VSP, and fragments thereof.

61. The immunogenic composition of claim 38 wherein a full length polynucleotide sequence encoding for the first Gram positive bacteria AI polypeptide is not present in a genome of a Gram positive bacteria comprising a full length polynucleotide sequence encoding for the second Gram positive bacteria AI polypeptide.

62. The immunogenic composition of claim 38 wherein polynucleotides encoding the first and the second Gram positive bacteria AI polypeptide are each present in genomes of more than one Gram positive bacteria serotype and strain isolate.

63. The immunogenic composition of claim 38 wherein the first and the second Gram positive bacteria AI polypeptides are of different Gram positive bacteria species.

64. The immunogenic composition of claim 38 wherein the first and the second Gram positive bacteria AI polypeptides are of the same Gram positive bacteria species.

65. The immunogenic composition of claim 38 wherein the first and the second Gram positive bacteria AI polypeptides are from different AI subtypes.

66. The immunogenic composition of claim 38 wherein the first and the second Gram positive bacteria AI polypeptides are from the same AI subtype.

67. The immunogenic composition of claim 38 wherein the first Gram positive bacteria AI polypeptide has detectable surface exposure on a first Gram positive bacteria strain or serotype but not a second Gram positive bacteria strain or subtype and the second Gram positive bacteria AI polypeptide has detectable surface exposure on the second Gram positive bacteria strain or serotype but not the first Gram positive bacteria strain or serotype.

68. The immunogenic composition of claim 38 wherein the Gram positive bacteria are S. pneumoniae, S. mutans, E. faecalis, E. faecium, C. difficile, L. monocytogenes, or C. diphtheriae.

69. An immunogenic composition comprising one or both of GBS59^{DK21} and GBS59^{CJB110} polypeptides or fragments thereof.

70. The composition of claim 69 wherein the combination comprises GBS59^{DK21} and GBS59^{CJB110}.

71. The immunogenic composition of claim 1 further comprising one or more GBS polypeptides selected from the group consisting of GBS80, GBS104, GBS67²⁶⁰³, GBS67^{H36B}, GBS59²⁶⁰³, GBS59^{CJB111}, GBS59⁵¹⁵, GBS59^{H36B}, 01524 and 01523.

72. The immunogenic composition claim 1 further comprising one or more polypeptides not selected from an adhesin island.

73. The immunogenic composition of claim 72 wherein the one or more polypeptides are selected from the group consisting of: GBS293, GBS65, GBS97, GBS276, GBS84, GBS322, GBS147 and GBS325.

74. The immunogenic composition of claim 1 wherein the composition further comprising one or more immunoregulatory agents.

75. The immunogenic composition of claim 74 wherein the one or more immunoregulatory agents include an adjuvant.

76. The immunogenic composition of claim 1 which is a vaccine.

77. A method for making a composition comprising one or both of GBS59^{DK21} and GBS59^{CJB110} polypeptides or fragments thereof comprising bringing into association: (a) an immunological effective amount of one or both GBS59^{DK21} and GBS59^{CJB110} polypeptides; and (b) a pharmaceutically acceptable excipient.

78. A modified Gram positive bacterium adapted to produce increased levels of AI surface protein.

79. The modified Gram positive bacterium of claim 78 wherein the AI surface protein is in oligomeric form.

80. The modified Gram positive bacterium of claim 79 wherein the oligomeric form is a hyperoligomer.

81. The modified Gram positive bacterium of claim 78 which is a non-pathogenic Gram positive bacterium.

82. The modified Gram positive bacterium of claim 81 wherein the non-pathogenic Gram positive bacterium is Lactococcus lactis or S. gordonii.

83. A method for manufacturing an oligomeric adhesin island (AI) surface antigen comprising:
culturing a Gram positive bacterium that expresses an oligomeric AI surface antigen; and
isolating the expressed oligomeric AI surface antigen.

84. A method for manufacturing an oligomeric adhesin island (AI) surface antigen comprising:
culturing the Gram positive bacterium of claim 78; and
isolating the expressed oligomeric AI surface antigen.

85. A method of neutralizing a Streptococcal infection in a mammal comprising the step of administering to the mammal an effective amount of the immunogenic composition of claim 1 or antibodies which recognize the an immunogenic composition of claim 1.

86. The method of claim 85 wherein the Streptococcal infection is a GBS infection.

87. The method of claim 85 wherein the Streptococcal infection is a GAS infection.

88. The method of claim 85 wherein the Streptococcal infection is a S. pneumoniae infection.

89. A method of raising an immune response in a mammal against a Streptococcal infection comprising administering to the mammal an effective amount of the immunogenic composition claim 1 or antibodies which recognize the an immunogenic composition of claim 1.

90. The method of claim 89 wherein the Streptococcal infection is a GBS infection.

91. The method of claim 89 wherein the Streptococcal infection is a GAS infection.

92. The method of claim 89 wherein the Streptococcal infection is a S. pneumoniae infection.

93-95. (canceled)
